(19) United States
(12) Patent Application Publication
(10) Pub. No.: US 2021/0198305 A1
(43) Pub. Date: Jul. 1, 2021

(54) OLIGONUCLEOTIDE COMPOSITIONS AND METHODS OF USE THEREOF

(71) Applicants: WAVE LIFE SCIENCES LTD., Singapore (SG); Pfizer Inc., New York, NY (US)

(72) Inventors: Chandra Vargeese, Schwenksville, PA (US); Naoki Iwamoto, Brighton, MA (US); David Charles Donnell Butler, Medford, MA (US); Subramanian Marappan, Acton, MA (US); Genliang Lu, Winchester, MA (US); Jason Jingxin Zhang, Walpole, MA (US); Vinod Vathipadiekal, Stoneham, MA (US); Maria David Frank-Kamenetsky, Brookline, MA (US); Luciano Henrique Apponi, Chelsea, MA (US); Hanna Maria Wisniewska, Providence, RI (US); Xiayun Cheng, Old Saybrook, CT (US); Young Jin Cho, Belmont, MA (US)

(21) Appl. No.: 16/618,003

(22) PCT Filed: Jun. 1, 2018
(86) PCT No.: PCT/US18/35712

§ 371 Date: Nov. 27, 2019

Related U.S. Application Data
(60) Provisional application No. 62/670,702, filed on May 11, 2018.
(60) Provisional application No. 62/514,769, filed on Jun. 2, 2017.

(51) Int. Cl. C07H 17/04; C07H 19/04; C12N 15/113

(52) U.S. Cl. CPC C07H 17/04; C12N 2310/315; C12N 15/113; C07H 19/04

(57) ABSTRACT

Among other things, the present disclosure provides designed APOC3 oligonucleotides, compositions, and methods thereof. In some embodiments, provided oligonucleotide compositions provide improved single-stranded RNA interference and/or RNase H-mediated knockdown. Among other things, the present disclosure encompasses the recognition that structural elements of oligonucleotides, such as base sequence, chemical modifications (e.g., modifications of sugar, base, and/or internucleotidic linkages) or patterns thereof, conjugation with additional chemical moieties, and/or stereochemistry [e.g., stereochemistry of backbone chiral centers (chiral internucleotidic linkages)], and/or patterns thereof, can have significant impact on oligonucleotide properties and activities, e.g., RNA interference (RNAi) activity, stability, delivery, etc. In some embodiments, the present disclosure provides methods for treatment of diseases using provided oligonucleotide compositions, for example, in RNA interference and/or RNase H-mediated knockdown.

### CROSS-REFERENCE TO RELATED APPLICATIONS

This application is a U.S. National Stage Application of International PCT Application No. PCT/US2018/035712, filed Jun. 1, 2018, which claims priority to United States Provisional Application Nos. 62/514,769, filed Jun. 2, 2017, and 62/670,702, filed May 11, 2018, the entirety of each of which is incorporated herein by reference.

### SEQUENCE LISTING

The instant application contains a Sequence Listing which has been submitted electronically in ASCII format and is hereby incorporated by reference in its entirety. Said ASCII copy, created on Nov. 27, 2019, is named SL.txt and is 590,014 bytes in size.

### BACKGROUND

Oligonucleotides which target APOC3 (APOC3 oligonucleotides) are useful in various applications, e.g., therapeutic applications. The use of naturally-occurring nucleic acids (e.g., unmodified DNA or RNA) can be limited, for example, by their susceptibility to endo- and exo-nucleases.

### SUMMARY

Among other things, the present disclosure encompasses the recognition that controlling structural elements of APOC3 oligonucleotides, such as chemical modifications (e.g., modifications of a sugar, base and/or internucleotidic linkage) or patterns thereof, alterations in stereochemistry (e.g., stereochemistry of a backbone chiral internucleotidic linkage) or patterns thereof, and/or conjugation with an additional chemical moiety (e.g., a lipid moiety, a targeting moiety, carbohydrate moiety, a moiety that binds to a asialoglycoprotein receptor or ASGPR, e.g., a GalNAc moiety, etc.) can have a significant impact on APOC3 oligonucleotide properties and/or activities. In some embodiments, the properties and/or activities include, but are not limited to, participation in, direction of a decrease in expression, activity or level of an APOC3 gene or a gene product thereof, mediated, for example, by RNA interference (RNAi interference), single-stranded RNA interference (ssRNAi), RNase H-mediated knockdown, steric hindrance of translation, etc.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting.

In some embodiments, the present disclosure encompasses the recognition that stereochemistry, particularly stereochemistry of backbone chiral centers, can unexpectedly improve properties of APOC3 oligonucleotides. In contrast to many prior observations that some structural elements that increase stability can also lower activity, for example, RNA interference, the present disclosure demonstrates that control of stereochemistry can, surprisingly, increase stability while not significantly decreasing activity.

In some embodiments, the present disclosure provides oligonucleotides having certain 5′-end structures.

In some embodiments, the present disclosure provides 5′-end structures that, when used in accordance with the present disclosure, can provided oligonucleotides with high biological activities, e.g., RNAi activity.

In some embodiments, the present disclosure encompasses the recognition that various additional chemical moieties, such as lipid moieties and/or carbohydrate moieties, when incorporated into oligonucleotides, can improve one or more APOC3 oligonucleotide properties, such as knock down of the APOC3 target gene or a gene product thereof. In some embodiments, an additional chemical moiety is optional. In some embodiments, an APOC3 oligonucleotide can comprise more than one additional chemical moiety. In some embodiments, an APOC3 oligonucleotide can comprise two or more additional chemical moieties, wherein the additional chemical moieties are identical or non-identical, or of the same category (e.g., targeting moiety, carbohydrate moiety, a moiety that binds to ASPGR, lipid moiety, etc.) or not of the same category. In some embodiments, certain additional chemical moieties facilitate delivery of oligonucleotides to desired cells, tissues and/or organs. In some embodiments, certain additional chemical moieties facilitate internalization of oligonucleotides and/or increase oligonucleotide stability.

In some embodiments, the present disclosure demonstrates that certain provided structural elements, technologies and/or features are particularly useful for APOC3 oligonucleotides that participate in and/or direct RNAi mechanisms (e.g., RNAi agents). Regardless, however, the teachings of the present disclosure are not limited to oligonucleotides that participate in or operate via any particular mechanism. In some embodiments, the present disclosure pertains to any oligonucleotide which operates through any mechanism, and which comprises any sequence, structure or format (or portion thereof) described herein. In some embodiments, the present disclosure provides an APOC3 oligonucleotide which operates through any mechanism, and which comprises any sequence, structure or format (or portion thereof) described herein, including, but not limited to, any 5′-end structure; 5′-end region; a first region (including but not limited to, a seed region); a second region (including, but not limited to, a post-seed region); and a 3′-end region (which can be a 3′-terminal dinucleotide and/or a 3′-end cap); an optional additional chemical moiety (including but not limited to a targeting moiety, a carbohydrate moiety, a moiety that binds APGR, and a lipid moiety); stereochemistry or patterns of stereochemistry; modification or pattern of modification; internucleotidic linkage or pattern of internucleotidic linkages; modification of sugar(s) or pattern of modifications of sugars; modification of base(s) or patterns of modifications of bases. In some embodiments, provided oligonucleotides may participate in (e.g., direct) RNAi mechanisms. In some embodiments, provided oligonucleotides may participate in RNase H (ribonuclease H) mechanisms. In some embodiments, provided oligonucleotides may act as translational inhibitors (e.g., may provide steric blocks of translation). In some embodiments, provided oligonucleotides may be therapeutic.

In some embodiments, an APOC3 target sequence is a sequence to which an APOC3 oligonucleotide as described herein binds. In many embodiments, a target sequence is identical to, or is an exact complement of, a sequence of a provided oligonucleotide, or of consecutive residues therein (e.g., a provided oligonucleotide includes a target-binding sequence that is identical to, or an exact complement of, a target sequence). In some embodiments, a target-binding sequence is an exact complement of a target sequence of a transcript (e.g., pre-mRNA, mRNA, etc.). A target-binding sequence/target sequence can be of various lengths to provided oligonucleotides with desired activities and/or properties. In some embodiments, a target binding sequence/target sequence comprises 5-50 bases. In some embodiments, a small number of differences/mismatches is tolerated between (a relevant portion of) an APOC3 oligonucleotide and its target sequence, including but not limited to the 5′ and/or 3′-end regions of the target and/or oligonucleotide sequence. In many embodiments, a target sequence is present within a transcript (e.g., an mRNA and/or a pre-mRNA) produced from a target gene.

Unless otherwise noted, all sequences (including, but not limited to base sequences and patterns of chemistry, modification, and/or stereochemistry) are presented in 5′ to 3′ order.

In some embodiments, the present disclosure provides compositions and methods related to an APOC3 oligonucleotide which is specific to a target and which has or comprises the base sequence of any oligonucleotide disclosed herein, or a region of at least 15 contiguous nucleotides of the base sequence of any oligonucleotide disclosed herein, wherein the first nucleotide of the base sequence or the first nucleotide of the at least 15 contiguous nucleotides can be optionally replaced by T or DNA T. In some embodiments, the oligonucleotide is capable of directing ssRNAi.

In some embodiments, the present disclosure provides an APOC3 oligonucleotide composition comprising a first plurality of oligonucleotides which have a common base sequence and comprise one or more internucleotidic linkage, sugar, and/or base modifications.

In some embodiments, a nucleotide is a natural nucleotide. In some embodiments, a nucleotide is a modified nucleotide. In some embodiments, a nucleotide is a nucleotide analog. In some embodiments, a base is a modified base. In some embodiments, a base is protected nucleobase, such as a protected nucleobase used in oligonucleotide synthesis. In some embodiments, a base is a base analog. In some embodiments, a sugar is a modified sugar. In some embodiments, a sugar is a sugar analog. In some embodiments, an internucleotidic linkage is a modified internucleotidic linkage. In some embodiments, a nucleotide comprises a base, a sugar, and an internucleotidic linkage, wherein each of the base, the sugar, and the internucleotidic linkage is independently and optionally naturally-occurring or non-naturally occurring. In some embodiments, a nucleoside comprises a base and a sugar, wherein each of the base and the sugar is independently and optionally naturally-occurring or non-naturally occurring. Non-limiting examples of nucleotides include DNA (2′-deoxy) and RNA (2′-OH) nucleotides; and those which comprise one or more modifications at the base, sugar and/or internucleotidic linkage. Non-limiting examples of sugars include ribose and deoxyribose; and ribose and deoxyribose with 2′-modifications, including but not limited to 2′-F, LNA, 2′-OMe, and 2′-MOE modifications. In some embodiments, an internucleotidic linkage can have a structure of Formula I as described in the present disclosure. In some embodiments, an internucleotidic linkage is a moiety which does not a comprise a phosphorus but serves to link two natural or non-natural sugars.

In some embodiments, the present disclosure provides a chirally controlled APOC3 oligonucleotide composition that directs a greater decrease of the expression, activity and/or level of an APOC3 gene or a gene product thereof, single-stranded RNA interference and/or RNase H-mediated knockdown, when compared to a reference condition, e.g., absence of the composition, or presence of a reference composition (e.g., a stereorandom composition of oligonucleotides having the same base sequence and chemical modifications).

In some embodiments, an APOC3 oligonucleotide composition comprising a plurality of oligonucleotides is stereorandom in that oligonucleotides of the plurality do not share a common stereochemistry at any chiral internucleotidic linkage. In some embodiments, an APOC3 oligonucleotide composition comprising a plurality of oligonucleotides is chirally controlled in that oligonucleotides of the plurality share a common stereochemistry at one or more chiral internucleotidic linkages. In some embodiments, an APOC3 oligonucleotide composition comprising a first plurality of oligonucleotides which is chirally controlled has a decreased susceptibility to endo- and exo-nucleases relative to an APOC3 oligonucleotide composition comprising a first plurality of oligonucleotides which is stereorandom.

In some embodiments, a composition comprises a multimer of two or more of any: APOC3 oligonucleotides of a first plurality and/or oligonucleotides of a second plurality, wherein the oligonucleotides of the first and second plurality can independently direct knockdown of the same or different targets independently via RNA interference and/or RNase H-mediated knockdown.

In some embodiments, an APOC3 oligonucleotide composition comprising a plurality of oligonucleotides (e.g., a first plurality of oligonucleotides) is chirally controlled in that oligonucleotides of the plurality share a common stereochemistry independently at one or more chiral internucleotidic linkages. In some embodiments, oligonucleotides of the plurality share a common stereochemistry configuration at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chiral internucleotidic linkages, each of which is independently Rp or Sp In some embodiments, oligonucleotides of the plurality share a common stereochemistry configuration at each chiral internucleotidic linkages. In some embodiments, a chiral internucleotidic linkage where a predetermined level of oligonucleotides of a composition share a common stereochemistry configuration (independently Rp or Sp) is referred to as a chirally controlled internucleotidic linkage.

In some embodiments, at least 5 internucleotidic linkages are chirally controlled; in some embodiments, at least 10 internucleotidic linkages are chirally controlled; in some embodiments, at least 15 internucleotidic linkages are chirally controlled; in some embodiments, each chiral internucleotidic linkage is chirally controlled.

In some embodiments, the present disclosure provides an APOC3 oligonucleotide composition comprising a first plurality of APOC3 oligonucleotides which share:

1) a common base sequence;
2) a common pattern of backbone linkages; and
3) a common pattern of backbone chiral centers, which composition is a substantially pure preparation of a single oligonucleotide in that a predetermined level of the oligonucleotides in the composition have the common base sequence and length, the common pattern of backbone linkages, and the common pattern of backbone chiral centers.

In some embodiments, the common pattern of backbone chiral centers comprises at least one internucleotidic linkage comprising a chirally controlled chiral center.

In some embodiments, levels of oligonucleotides and/or diastereopurity can be determined by analytical methods, e.g., chromatographic, spectrometric, spectroscopic methods or any combinations thereof.

Among other things, the present disclosure encompasses the recognition that stereorandom APOC3 oligonucleotide preparations contain a plurality of distinct chemical entities that differ from one another, e.g., in the stereochemical structure (or stereochemistry) of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence and/or chemical modifications, they are different chemical entities at least due to their different backbone stereochemistry, and they can have, as demonstrated herein, different properties, e.g., sensitivity to nucleases, activities, distribution, etc. In some embodiments, a particular stereoisomer may be defined, for example, by its base sequence, its length, its pattern of backbone linkages, and its pattern of backbone chiral centers. In some embodiments, the present disclosure demonstrates that improvements in properties and activities achieved through control of stereochemistry within an APOC3 oligonucleotide can be comparable to, or even better than those achieved through use of chemical modification.

In some embodiments, an APOC3 oligonucleotide comprises, in 5′ to 3′ order, a 5′-end region, a seed region, a post-seed region, and a 3-end region, optionally further comprising an additional chemical moiety.

In some embodiments, a 5′-end region is the entire portion of an APOC3 oligonucleotide which is 5′ to the seed region. In some embodiments, a 3′-end region is the entire portion of an APOC3 oligonucleotide which is 3′ to the post-seed region.

In some embodiments, a 5′-end structure is a 5′-end group.

In some embodiments, a 5′-end structure comprises a 5′-end group.

In some embodiments, a provided oligonucleotide can comprise a 5′-end region, 5′-end structure, 5′-end group, 5′-end nucleoside, or 5′-end nucleotide described herein or known in the art.

In some embodiments, a 5′-end structure, a 5′-end region, 5′ nucleotide moiety, seed region, post-seed region, 3′-terminal dinucleotide and/or 3′-end cap independently have any structure described herein or known in the art. In some embodiments, any structure for a 5′-end described herein or known in the art and/or any structure for a 5′ nucleotide moiety described herein or known in the art and/or any structure for a seed region described herein or known in the art and/or any structure for a post-seed region described herein or known in the art and/or any structure for a 3′-terminal dinucleotide described herein or known in the art and/or any structure for a 3′-end cap described herein or known in the art can be combined.

In some embodiments, a provided oligonucleotide comprises one or more blocks. In some embodiments, a provided oligonucleotide comprise one or more blocks, wherein a block comprises one or more consecutive nucleosides, and/or nucleotides, and/or sugars, or bases, and/or internucleotidic linkages. In some embodiments, a block encompasses an entire seed region or a portion thereof. In some embodiments, a block encompasses an entire post-seed region or a portion thereof.

In some embodiments, provided oligonucleotides are blockmers.

In some embodiments, provided oligonucleotides are altmers comprising alternating blocks. In some embodiments, a blockmer or an altmer can be defined by chemical modifications (including presence or absence), e.g., base modifications, sugar modification, internucleotidic linkage modifications, stereochemistry, etc., or patterns thereof.

In some embodiments, provided oligonucleotides comprise one or more sugar modifications. In some embodiments, a sugar modification is at the 2′-position. In some embodiments, a sugar modification is selected from: 2′-F, 2′-OMe, and 2′-MOE. 2′-F is also designated 2′ Fluoro. 2′-OMe is also designated 2′-O-Methyl. 2′-MOE is also designated 2′-Methoxyethyl or MOE.

In some embodiments, an APOC3 oligonucleotide comprises only two 2′-F. In some embodiments, an APOC3 oligonucleotide comprises only two 2′-F, wherein the two nucleotides are at the 2nd and 14th positions.

In some embodiments, an APOC3 oligonucleotide comprises only two 2′-F, wherein the two nucleotides are at the 2nd and 14th positions, and wherein the first nucleotide is 2′-deoxy.

In some embodiments, an APOC3 oligonucleotide comprises only two 2′-F, wherein the two nucleotides are at the 2nd and 14th positions, and wherein the first nucleotide is 2′-deoxy T.

In some embodiments, an APOC3 oligonucleotide comprises only two 2′-F, wherein the two nucleotides are at the 2nd and 14th positions, and wherein the first nucleotide is 2′-deoxy, and the 5′-end structure is —OH.

In some embodiments, an APOC3 oligonucleotide comprises only two 2′-F, wherein the two nucleotides are at the 2nd and 14th positions, and wherein the first nucleotide is 2′-deoxy T, and the 5′-end structure is —OH.

In some embodiments herein, in reference to an APOC3 oligonucleotide, “first” (e.g., first nucleotide) refers to the 5′ end of the oligonucleotide, and “last” or “end” (e.g., last nucleotide or end nucleotide) refers to the 3′ end.

In some embodiments, provided oligonucleotides comprise sugars with a particular modification which alternate with sugars with no modification or a different modification. In some embodiments, sugars with a particular modification appear in one or more blocks.

In some embodiments, provided oligonucleotides comprise one or more blocks comprising sugars with a particular 2′ modification which alternate with sugars which independently have no modification or have a different modification. In some embodiments, provided oligonucleotides comprise one or more blocks comprising sugars with a 2′-F modification which alternate with sugars which independently have no modification or have a different modification. In some embodiments, provided oligonucleotides comprise one or more blocks comprising sugars with a 2′-OMe modification which alternate with sugars which independently have no modification or a different modification. In some embodiments, provided oligonucleotides one or more blocks comprising sugars with a 2′-OMe modification which alternate with sugars with a 2′-F modification.

In some embodiments, a block of sugars has or comprises a pattern of 2′-modifications of any of: ff, fffm, fffmm, fffmmm, fffmmmm, fffiummmm, fffmmmmmm, fffmmmmmmf, fffmmmmmmff, fffmmmmmmffm, fffmmmmmmffmm, fffmmmmmmffmmf, fffmmmmmmffmmfm, fffmmmmmmffmmfmf, fffmmmmmmffmmfmfm, fffmmmmmmffmmfmfmf, fffmmmmmmffmmfmfmfm, fffmmmmmmffmmfmfmfmm, fffmmmmmmffmmfmfmfmmm, ffmmffmm, ffmmmmmmffmmfmfmfmmm, fmfmfmfmfmfmfm, fmfmfmfmfmfmfmf, fmfmfmfmfmfmfmfm, fmfmfmfmfmfmfmfmf, fmfmfmfmfmfmfmfmfm, fmfmfmfmfmfmfmfmfmf, fmfmfmfmfmfmfmfmfmfm, fmfmfmfmfmfmfmfmfmfmm, fmfmfmfmfmfmfmfmfmm, fmfmfmfmfmfmfmfmm, fmfmfmfmfmfmfmm, fmfmfmfmfmfmmm, fmmffmm, fmmmmmmffmmfmfmfmmm, mff, mffm, mffmf, mffmff, mffmffm, mffmmffmm, mfmfm, mfmfmfmfmfffmfmfmfmmm, mfmfmfmfmfmfmfm, mfmfmfmfmfmfmfmfmm, mfmfmfmfmfmfmfmfmfmmm, mfmfmfmfmfmfmfmm, mfmfmfmfmfmfmfmm, mfmfmfmfmfmfmm, mfmfmfmfmfmfmmm, mfmfmfmfmfmmm, mfmfmfmfmfmmmfm, mfmfmfmfmfmmmmm, mfmfmfmfmmm, mfmfmfmfmmmfmfm, mfmfmfmfmmmfmmm, mfmfmfmfmmmmmfm, mfmfmfmmm, mfmfmfmmmfmfmfm, mfmfmfmmmfmfmmm, mfmfmfmmmfmmmfm, mfmfmfmmmmmfmfm, mfmfmmm, mfmfmmmfmfmfmfm, mfmfmmmfmfmfmmm, mfmfmmmfmfmmmfm, mfmfmmmfmmmfmfm, mfmfmmmmmfmfmfm, mfmmm, mfmmmfmfmfmfmfm, mfmmmfmfmfmfmmm, mfmmmfmfmfmmmfm, mfmmmfmfmmmfmfm, mfmmmfmmmfmfmfm, mfmmmfmmmfmfmfm, mfmmmmmfmfmfmfm, mmffm, mmffmm, mmffmm, mmffmmf, mmffmmff, mmffmmffm, mmffmmffmm, mmffmmfmfmfmmm, mmm, mmmffmmfmfmfmmm, mmmfmfmfmfmfmfm, mmmfmfmfmfmfmmm, mmmfmfmfmfmmmfm, mmmfmfmfmmmfmfm, mmmfmfmmmfmfmfm, mmmfmmmfmfmfmfm, mmmmffmmfmfmfmfmmm, mmm, mmmm, mmmmm, mmmmmffmmfmfmfmmm, mmmmmfmfmfmfmfm, mmmmmm, mmmmmmffmmfmfmfmmm, mfmf, mfmf, mfmfmf, fmfm, fmfmfm, fmfmfmf, dfdf, dfdfdf, dfdfdfdf, fdfd, fdfdfd, fdfdfdfd, dfdfmfmf, dfmfmf, mfdfmf, or dfmfdf, wherein m indicates a 2′-OMe, f indicates a 2′-F, and d indicates no substitution at 2′-position. In some embodiments, a seed region and/or post-seed region can comprise a block of sugar modifications.

In some embodiments, a block is a stereochemistry block. In some embodiments, a block is an Rp block in that each internucleotidic linkage of the block is Rp. In some embodiments, a seed region-block is an Rp block. In some embodiments, a post-seed region-block is an Rp block. In some embodiments, a block is an Sp block in that each internucleotidic linkage of the block is Sp. In some embodiments, a seed region-block is an Sp block. In some embodiments, a post-seed region-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage of the block is a natural phosphate linkage.

In some embodiments, a seed region-block is an Sp block wherein each sugar moiety comprises a 2′-F modification. In some embodiments, a seed region-block is an Sp block wherein each of internucleotidic linkage is a modified internucleotidic linkage and each sugar moiety comprises a 2′-F modification. In some embodiments, a seed region-block is an Sp block wherein each of internucleotidic linkage is a phosphorothioate linkage and each sugar moiety comprises a 2′-F modification. In some embodiments, a seed region-block comprises 4 or more nucleoside units. In some embodiments, a nucleoside unit is a nucleoside. In some embodiments, a seed region-block comprises 5 or more nucleoside units. In some embodiments, a seed region-block comprises 6 or more nucleoside units. In some embodiments, a seed region-block comprises 7 or more nucleoside units. In some embodiments, a post-seed region-block is an Sp block wherein each sugar moiety comprises a 2′-F modification. In some embodiments, a post-seed region-block is an Sp block wherein each of internucleotidic linkage is a modified internucleotidic linkage and each sugar moiety comprises a 2′-F modification. In some embodiments, a post-seed region-block is an Sp block wherein each of internucleotidic linkage is a phosphorothioate linkage and each sugar moiety comprises a 2′-F modification. In some embodiments, a post-seed region-block comprises 4 or more nucleoside units. In some embodiments, a post-seed region-block comprises 5 or more nucleoside units. In some embodiments, a post-seed region-block comprises 6 or more nucleoside units. In some embodiments, a post-seed region-block comprises 7 or more nucleoside units. In some embodiments, a seed region and/or post-seed region can comprise a block. In some embodiments, a seed region and/or post-seed region comprises a stereochemistry block.

In some embodiments, the present disclosure provides an APOC3 oligonucleotide composition comprising a first plurality of oligonucleotides which:

1) have a common base sequence; and

2) comprise one or more modified sugar moieties and modified internucleotidic linkages.

In some embodiments, the present disclosure provides an APOC3 oligonucleotide composition comprising a first plurality of oligonucleotides capable of directing single-stranded RNA interference which:

1) have a common base sequence complementary to a target sequence in a transcript; and

2) comprise one or more modified sugar moieties and modified internucleotidic linkages.

In some embodiments, a reference condition is absence of the composition. In some embodiments, a reference condition is presence of a reference composition. Example reference compositions comprising a reference plurality of oligonucleotides are extensively described in this disclosure. In some embodiments, oligonucleotides of the reference plurality have a different structural elements (chemical modifications, stereochemistry, etc.) compared with oligonucleotides of the first plurality in a provided composition. In some embodiments, a provided oligonucleotide composition comprising a first plurality of oligonucleotide is chirally controlled in that the first plurality of oligonucleotides comprise one or more chirally controlled internucleotidic linkages. In some embodiments, a provided oligonucleotide composition comprising a first plurality of oligonucleotide is chirally controlled in that the first plurality of oligonucleotides comprise 1-20 chirally controlled internucleotidic linkages. In some embodiments, the first plurality of oligonucleotides comprise 2, 3, 4, 5, 6, 7, 8, 9, 10, 11, 12, 13, 14, or 15 chirally controlled internucleotidic linkages. In some embodiments, a reference composition is a stereorandom preparation of oligonucleotides having the same chemical modifications. In some embodiments, a reference composition is a mixture of stereoisomers while a provided composition is a single-stranded RNAi agent of one stereoisomer. In some embodiments, oligonucleotides of the reference plurality have the same base sequence as oligonucleotide of the first plurality in a provided composition. In some embodiments, oligonucleotides of the reference plurality have the same chemical modifications as oligonucleotide of the first plurality in a provided composition. In some embodiments, oligonucleotides of the reference plurality have the same sugar modifications as oligonucleotide of the first plurality in a provided composition. In some embodiments, oligonucleotides of the reference plurality have the same base modifications as oligonucleotide of the first plurality in a provided composition. In some embodiments, oligonucleotides of the reference plurality have the same internucleotidic linkage modifications as oligonucleotide of the first plurality in a provided composition. In some embodiments, oligonucleotides of the reference plurality have the same base sequence and the same chemical modifications as oligonucleotide of the first plurality in a provided composition. In some embodiments, oligonucleotides of the reference plurality have the same stereochemistry as oligonucleotide of the first plurality in a provided composition but different chemical modifications, e.g., base modification, sugar modification, internucleotidic linkage modifications, etc.

In some embodiments, the present disclosure provides a composition comprising an APOC3 oligonucleotide, wherein the oligonucleotide is complementary or substantially complementary to a target RNA sequence, has a length of about 15 to about 49 total nucleotides, wherein the oligonucleotide comprises at least one non-natural base, sugar and/or internucleotidic linkage.

In some embodiments, the present disclosure provides an APOC3 oligonucleotide composition comprising a single-stranded RNAi agent, wherein the single-stranded RNAi agent is complementary or substantially complementary to a target RNA sequence, has a length of about 15 to about 49 total nucleotides, and is capable of directing target-specific RNA interference, wherein the single-stranded RNAi agent comprises at least one non-natural base, sugar and/or internucleotidic linkage.

In some embodiments, the length is 15 to 49, about 17 to about 49, 17 to 49, about 19 to about 29, 19 to 29, about 19 to about 25, 19 to 25, about 19 to about 23, or 19 to 23 total nucleotides.

In some embodiments, the present disclosure provides an APOC3 oligonucleotide composition comprising a first plurality of oligonucleotides which:

1) have a common base sequence complementary or substantially complementary to a target sequence in a transcript; and

2) comprise one or more modified sugar moieties and modified internucleotidic linkages,

the oligonucleotide composition being characterized in that, when it is contacted with the transcript, knockdown of the transcript is improved relative to that observed under reference conditions selected from the group consisting of absence of the composition, presence of a reference composition, and combinations thereof.

In some embodiments, the present disclosure provides an APOC3 oligonucleotide composition comprising a first plurality of oligonucleotides capable of directing single-stranded RNA interference which:

1) have a common base sequence complementary to a target sequence in a transcript; and

2) comprise one or more modified sugar moieties and modified internucleotidic linkages,

the oligonucleotide composition being characterized in that, when it is contacted with the transcript in a RNA interference system, RNAi-mediated knockdown of the transcript is improved relative to that observed under reference conditions selected from the group consisting of absence of the composition, presence of a reference composition, and combinations thereof.

In some embodiments, the present disclosure provides an APOC3 oligonucleotide composition comprising a first plurality of oligonucleotides, wherein oligonucleotides of the first plurality are of a particular oligonucleotide type defined by:

1) base sequence;

2) pattern of backbone linkages;

3) pattern of backbone chiral centers; and

4) pattern of backbone phosphorus modifications.

In some embodiments, the present disclosure provides an APOC3 oligonucleotide composition comprising a first plurality of oligonucleotides capable of directing single-stranded RNA interference, wherein oligonucleotides of the first plurality are of a particular oligonucleotide type defined by:

1) base sequence;

2) pattern of backbone linkages;

3) pattern of backbone chiral centers; and

4) pattern of backbone phosphorus modifications.

In some embodiments, the present disclosure provides an APOC3 oligonucleotide composition comprising a first plurality of oligonucleotides of an APOC3 oligonucleotide type, wherein the oligonucleotide type is defined by:

1) base sequence;

2) pattern of backbone linkages;

3) pattern of backbone chiral centers; and

4) pattern of backbone phosphorus modifications,

which composition is chirally controlled in that it is enriched, relative to a substantially racemic preparation of oligonucleotides having the same base sequence, for oligonucleotides of the particular oligonucleotide type,

the oligonucleotide composition being characterized in that, when it is contacted with the transcript, knockdown of the transcript is improved relative to that observed under reference conditions selected from the group consisting of absence of the composition, presence of a reference composition, and combinations thereof.

In some embodiments, the present disclosure provides an APOC3 oligonucleotide composition comprising a first plurality of oligonucleotides which are capable of directing single-stranded RNA interference and are of an APOC3 oligonucleotide type, wherein the oligonucleotide type is defined by:

1) base sequence;

2) pattern of backbone linkages;

3) pattern of backbone chiral centers; and

4) pattern of backbone phosphorus modifications,

which composition is chirally controlled in that it is enriched, relative to a substantially racemic preparation of oligonucleotides having the same base sequence, for oligonucleotides of the particular oligonucleotide type,

the oligonucleotide composition being characterized in that, when it is contacted with the transcript in a RNA interference system, RNAi-mediated knockdown of the transcript is improved relative to that observed under reference conditions selected from the group consisting of absence of the composition, presence of a reference composition, and combinations thereof.

In some embodiments, a provided oligonucleotide has any of the Formats illustrated in FIG. 1, or any structural element illustrated in any of the Formats illustrated in FIG. 1.

In some embodiments, a provided single-stranded RNAi agent has any of the Formats illustrated in FIG. 1, or any structural element illustrated in any of the Formats illustrated in FIG. 1.

Among other things, the present disclosure presents data showing that various oligonucleotides of the disclosed Formats are capable of directing a decrease in the expression and/or level of a target gene or its gene product, when targeted against any of several different sequences, in any of several different genes. In some embodiments, the present disclosure presents data showing that various RNAi agents of the disclosed Formats are capable of directing RNA interference against any of many different sequences, in any of many different genes.

In some embodiments, an APOC3 oligonucleotide having any of the structures described and/or illustrated herein is capable of directing RNA interference. In some embodiments, an APOC3 oligonucleotide having any of the structures described and/or illustrated herein is capable of directing RNase H-mediated knockdown. In some embodiments, an APOC3 oligonucleotide having any of the structures described and/or illustrated herein is capable of directing RNA interference and/or RNase H-mediated knockdown. In some embodiments, an APOC3 oligonucleotide comprises any structural element of any oligonucleotide described herein, or any Format described herein or illustrated in FIG. 1. In some embodiments, an APOC3 oligonucleotide comprises any structural element of any oligonucleotide described herein, or any Format described herein or illustrated in FIG. 1 and is capable of directing RNA interference. In some embodiments, an APOC3 oligonucleotide comprises any structural element of any oligonucleotide described herein, or any Format described herein or illustrated in FIG. 1 and is capable of directing RNase H-mediated knockdown. In some embodiments, an APOC3 oligonucleotide comprises any structural element of any oligonucleotide described herein, or any Format described herein or illustrated in FIG. 1 and is capable of directing RNA interference and/or RNase H-mediated knockdown.

In some embodiments, a RNAi agent comprises any one or more of: a 5′-end structure, a 5′-end region, a seed region, a post-seed region, and a 3′-end region, and an optional additional chemical moiety. In some embodiments, a seed region is any seed region described herein or known in the art. In some embodiments, a post-seed region can be any region between a seed region and a 3′-end region described herein or known in the art. In some embodiments, a 3′-end region can be any 3′-end region described herein or known in the art. In some embodiments, any optional additional chemical moiety can be any optional additional chemical moiety described herein or known in the art. Any individual 5′-end structure, 5′-end region, seed region, post-seed region, 3′-end region, and optional additional chemical moiety described herein or known in the art can be combined, independently, with any other 5′-end structure, 5′-end region, seed region, post-seed region, 3′-end region, and optional additional chemical moiety described herein or known in the art. In some embodiments, as non-limiting examples, a region of a single-stranded RNAi agent is a 5′-end structure, a 5′-end region, a seed region, a post-seed region, a portion of a seed region, a portion of a post-seed region, or a 3′-terminal dinucleotide.

In some embodiments, the base sequence of a provided oligonucleotide consists of the base sequence of any oligonucleotide disclosed herein. In some embodiments, the base sequence of a provided oligonucleotide comprises the base sequence of any oligonucleotide disclosed herein. In some embodiments, the base sequence of a provided oligonucleotide comprises a sequence comprising the sequence of 15 contiguous bases of the base sequence of any oligonucleotide disclosed herein. In some embodiments, the base sequence of a provided oligonucleotide comprises a sequence comprising the sequence of 20 contiguous bases, with up to 5 mismatches, of the base sequence of any oligonucleotide disclosed herein.

In some embodiments, a provided oligonucleotide is capable of directing a decrease in the expression and/or level of a target gene or its gene product. In some embodiments, a provided oligonucleotide is capable of directing single-stranded RNAi interference. In some embodiments, a provided oligonucleotide is capable of directing RNase H-mediated knockdown. In some embodiments, a provided oligonucleotide is capable of directing single-stranded RNA interference and RNase H-mediated knockdown. In some embodiments, a oligonucleotide comprises a sequence which targets any transcript or gene targeted by a oligonucleotide disclosed herein.

In some embodiments, provided oligonucleotides target APOC3.

In some embodiments, provided oligonucleotides can be used to decrease or inhibit the activity, level and/or expression of an APOC3 gene or its gene product. In some embodiments, provided oligonucleotides can be used to decrease or inhibit the activity, level and/or expression of a gene or its gene product, wherein abnormal or excessive activity, level and/or expression of, a deleterious mutation in, or abnormal tissue or inter- or intracellular distribution of a gene or its gene product is related to, causes and/or is associated with a disorder. In some embodiments, provided oligonucleotides can be used to treat a disorder and/or to manufacture a medicament for the treatment of a disorder related to, caused and/or associated with the abnormal or excessive activity, level and/or expression or abnormal distribution of a gene or its gene product.

In some embodiments, the present disclosure pertains to methods of using oligonucleotides disclosed herein which are capable of targeting APOC3 and useful for treating and/or manufacturing a treatment for an APOC3-related disorder.

In some embodiments, an APOC3 oligonucleotide capable of targeting a gene comprises a base sequence which is a portion of or complementary or substantially complementary to a portion of the base sequence of the target gene. In some embodiments, a portion is at least 15 bases long. In some embodiments, a base sequence of a single-stranded RNAi agent can comprise or consist of a base sequence which has a specified maximum number of mismatches from a specified base sequence.

In some embodiments, a mismatch is a difference between the base sequence or length when two sequences are maximally aligned and compared. As a non-limiting example, a mismatch is counted if a difference exists between the base at a particular location in one sequence and the base at the corresponding position in another sequence. Thus, a mismatch is counted, for example, if a position in one sequence has a particular base (e.g., A), and the corresponding position on the other sequence has a different base (e.g., G, C or U). A mismatch is also counted, e.g., if a position in one sequence has a base (e.g., A), and the corresponding position on the other sequence has no base (e.g., that position is an abasic nucleotide which comprises a phosphate-sugar backbone but no base) or that position is skipped. A single-stranded nick in either sequence (or in the sense or antisense strand) may not be counted as mismatch, for example, no mismatch would be counted if one sequence comprises the sequence 5′-AG-3′, but the other sequence comprises the sequence 5′-AG-3′ with a single-stranded nick between the A and the G. A base modification is generally not considered a mismatch, for example, if one sequence comprises a C, and the other sequence comprises a modified C (e.g., 5mC) at the same position, no mismatch may be counted. In some embodiments, for purposes of counting mismatches, substitution of a T for U or vice versa is not considered a mismatch.

In some embodiments, an APOC3 oligonucleotide is complementary or totally or 100% complementary to a target sequence (e.g., a RNA, such as a mRNA), meaning that the base sequence of the oligonucleotide has no mismatches with a sequence which is fully complementary (e.g., base-pairs via Watson-Crick basepairing) to the target sequence. Without wishing to be bound by any particular theory, the disclosure notes that, for a single-stranded RNAi agent, it is not necessary for the 5′-end nucleotide moiety or the 3′-terminal dinucleotide to base-pair with the target. These may be mismatches. In addition, an antisense oligonucleotide or single-stranded RNAi agent can have a small number of internal mismatches and still direct a decrease in the expression and/or level of a target gene or its gene product and/or direct RNase H-mediated knockdown and/or RNA interference. If a first base sequence of an APOC3 oligonucleotide, (e.g., antisense oligonucleotide or single-stranded RNAi agent) has a small number of mismatches from a reference base sequence which is 100% complementary to a target sequence, then the first base sequence is substantially complementary to the target sequence. In some embodiments, an APOC3 oligonucleotide, (e.g., antisense oligonucleotide or single-stranded RNAi agent) can have a base sequence which is complementary or substantially complementary to a target sequence. In some embodiments, complementarity is determined based on Watson-Crick base pairs (guanine-cytosine and adenine-thymine/uracil), wherein guanine, cytosine, adenine, thymine, uracil may be optionally and independently modified but maintains their pairing hydrogen-bonding patters as unmodified. In some embodiments, a sequence complementary to another sequence comprises at least 5, 6, 7, 8, 9, 10, 11, 12, 13, 14, 15, 16, 17, 18, 19, 20 bases.

In some embodiments, an APOC3 oligonucleotide, oligonucleotide composition or oligonucleotide type has a common pattern of backbone linkages. In some embodiments, a common pattern of backbone linkages comprises at least 10 modified internucleotidic linkages.

In some embodiments, a common pattern of backbone linkages comprises at least 10 phosphorothioate linkages. In some embodiments, an APOC3 oligonucleotide, oligonucleotide composition or oligonucleotide type has a common pattern of backbone chiral centers. In some embodiments, a common pattern of backbone chiral centers comprises at least 1 internucleotidic linkage in the Sp configuration. In some embodiments, a common pattern of backbone chiral centers comprises at least 1 internucleotidic linkage which is phosphorothioate in the Sp configuration. In some embodiments, oligonucleotides in provided compositions have a common pattern of backbone phosphorus modifications. In some embodiments, a provided composition is an APOC3 oligonucleotide composition that is chirally controlled in that the composition contains a predetermined level of oligonucleotides of an individual oligonucleotide type, wherein an APOC3 oligonucleotide type is defined by:

1) base sequence;

2) pattern of backbone linkages;

3) pattern of backbone chiral centers; and

4) pattern of backbone phosphorus modifications.

As noted above and understood in the art, in some embodiments, base sequence of an APOC3 oligonucleotide may refer to the identity and/or modification status of nucleoside residues (e.g., of sugar and/or base components, relative to standard naturally occurring nucleotides such as adenine, cytosine, guanosine, thymine, and uracil) in the oligonucleotide and/or to the hybridization character (i.e., the ability to hybridize with particular complementary residues) of such residues.

In some embodiments, a particular oligonucleotide type may be defined by

1A) base identity;

1B) pattern of base modification;

1C) pattern of sugar modification;

2) pattern of backbone linkages;

3) pattern of backbone chiral centers; and

4) pattern of backbone phosphorus modifications.

Thus, in some embodiments, oligonucleotides of a particular type may share identical bases but differ in their pattern of base modifications and/or sugar modifications. In some embodiments, oligonucleotides of a particular type may share identical bases and pattern of base modifications (including, e.g., absence of base modification), but differ in pattern of sugar modifications.

In some embodiments, oligonucleotides of a particular type are chemically identical in that they have the same base sequence (including length), the same pattern of chemical modifications to sugar and base moieties, the same pattern of backbone linkages (e.g., pattern of natural phosphate linkages, phosphorothioate linkages, phosphorothioate triester linkages, and combinations thereof), the same pattern of backbone chiral centers (e.g., pattern of stereochemistry (Rp/Sp) of chiral internucleotidic linkages), and the same pattern of backbone phosphorus modifications (e.g., pattern of modifications on the internucleotidic phosphorus atom, such as —S⁻, and -L-R¹ of Formula I).

Among other things, the present disclosure provides oligonucleotide compositions and technologies for optimizing properties, e.g., improved single-stranded RNA interference, RNase H-mediated knockdown, etc. In some embodiments, the present disclosure provides methods for lowering immune response associated with administration of oligonucleotides and compositions thereof (i.e., of administering oligonucleotide compositions so that undesirable immune responses to oligonucleotides in the compositions are reduced, for example relative to those observed with a reference composition of nucleotides of comparable or identical nucleotide sequence). In some embodiments, the present disclosure provides methods for increasing binding to certain proteins by oligonucleotides and compositions thereof. In some embodiments, the present disclosure provides methods for increasing binding to certain proteins by oligonucleotides and compositions thereof. In some embodiments, the present disclosure provides methods for enhancing delivery of oligonucleotides and compositions thereof. Among other things, the present disclosure encompasses the recognition that optimal delivery of oligonucleotides to their targets, in some embodiments, involves balance of oligonucleotides binding to certain proteins so that oligonucleotides can be transported to the desired locations, and oligonucleotide release so that oligonucleotides can be properly released from certain proteins to perform their desired functions, for example, hybridization with their targets, cleavage of their targets, inhibition of translation, modulation of transcript processing, etc. As exemplified in this disclosure, the present disclosure recognizes, among other things, that improvement of oligonucleotide properties can be achieved through chemical modifications and/or stereochemistry.

In some embodiments, the present disclosure provides a method for treating or preventing a disease, comprising administering to a subject an APOC3 oligonucleotide composition described herein.

In some embodiments, a disease is one in which, after administering a provided composition, knocking down a target nucleic acid via single-stranded RNA interference can repair, restore or introduce a new beneficial function.

In some embodiments, a common sequence comprises a sequence selected from Table 1A. In some embodiments, a common sequence is a sequence selected from Table 1A. In some embodiments, a pattern of backbone chiral centers is selected from those described in Table 1A.

In some embodiments, the present disclosure provides a method comprising administering a composition comprising a first plurality of oligonucleotides, which composition displays improved delivery as compared with a reference composition comprising a plurality of oligonucleotides, each of which also has the common base sequence but which differs structurally from the oligonucleotides of the first plurality in that:

individual oligonucleotides within the reference plurality differ from one another in stereochemical structure; and/or

at least some oligonucleotides within the reference plurality have a structure different from a structure represented by the plurality of oligonucleotides of the composition.

In some embodiments, the present disclosure provides a method of administering an APOC3 oligonucleotide composition comprising a first plurality of oligonucleotides capable of directing a decrease in the expression and/or level of a target gene or its gene product and having a common nucleotide sequence, the improvement that comprises:

administering an APOC3 oligonucleotide comprising a first plurality of oligonucleotides that is characterized by improved delivery relative to a reference oligonucleotide composition of the same common nucleotide sequence.

In some embodiments, the present disclosure provides a method of administering an APOC3 oligonucleotide composition comprising a first plurality of oligonucleotides capable of directing single-stranded RNA interference and having a common nucleotide sequence, the improvement that comprises:

administering an APOC3 oligonucleotide comprising a first plurality of oligonucleotides that is characterized by improved delivery relative to a reference oligonucleotide composition of the same common nucleotide sequence.

In some embodiments, the present disclosure provides a single-stranded RNAi agent of an APOC3 oligonucleotide selected from any of the Tables, including but not limited to Table 1A, or otherwise disclosed herein. In some embodiments, the present disclosure provides a single-stranded RNAi agent of an APOC3 oligonucleotide selected from any of the Tables, including but not limited to Table 1A, or otherwise disclosed herein, wherein the oligonucleotide is conjugated to a lipid moiety.

In some embodiments, a provided oligonucleotide comprises a lipid moiety. In some embodiments, a lipid moiety is incorporated by conjugation with a lipid. In some embodiments, a lipid moiety is a fatty acid. In some embodiments, an APOC3 oligonucleotide is conjugated to a fatty acid. In some embodiments, a provided single-stranded RNAi agent further comprises a lipid. In some embodiments, a provided single-stranded RNAi agent comprises a lipid moiety conjugated at the 9^{th} or 11^{th} nucleotide (counting from the 5′-end). In some embodiments, an APOC3 oligonucleotide is conjugated at the base to a fatty acid. In some embodiments, a provided single-stranded RNAi agent comprises a lipid moiety. In some embodiments, a provided single-stranded RNAi agent comprises a lipid moiety conjugated at the base at the 9^{th} or 11^{th} nucleotide (counting from the 5′-end).

In some embodiments, a single-stranded RNAi agent is any one of the preceding compositions, further comprising one or more additional components.

In some embodiments, a provided oligonucleotide is capable of degrading a target transcript, e.g., RNA, through both a RNase H mechanism and a RNAi mechanism.

In some embodiments, conjugation of a lipid moiety to an APOC3 oligonucleotide improves at least one property of the oligonucleotide. In some embodiments, improved properties include increased activity (e.g., increased ability to direct a decrease in the expression and/or level of a target gene or its gene product and/or direct single-stranded RNA interference and/or direct RNase H-mediated knockdown) and/or improved distribution to a tissue. In some embodiments, a tissue is muscle tissue. In some embodiments, a tissue is skeletal muscle, gastrocnemius, triceps, heart or diaphragm. In some embodiments, improved properties include reduced hTLR9 agonist activity. In some embodiments, improved properties include hTLR9 antagonist activity. In some embodiments, improved properties include increased hTLR9 antagonist activity.

In general, properties of oligonucleotide compositions as described herein can be assessed using any appropriate assay.

Those of skill in the art will be aware of and/or will readily be able to develop appropriate assays for particular oligonucleotide compositions.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value to which it refers, or in one embodiment, of plus or minus 5%, or, in another embodiment, of plus or minus 2%. For the field of this disclosure, this level of approximation is appropriate unless the value is specifically stated to require a tighter range.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an alkyl group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an alkyl group, as defined herein, having one or more triple bonds.

Antisense: The term “Antisense”, as used herein, refers to a characteristic of an oligonucleotide or other nucleic acid having a base sequence complementary or substantially complementary to a target nucleic acid to which it is capable of hybridizing. In some embodiments, a target nucleic acid is a target gene mRNA. In some embodiments, hybridization is required for or results in at one activity, e.g., a decrease in the level, expression or activity of the target nucleic acid or a gene product thereof. The term “antisense oligonucleotide”, as used herein, refers to an oligonucleotide complementary to a target nucleic acid. In some embodiments, an antisense oligonucleotide is capable of directing a decrease in the level, expression or activity of the target nucleic acid or a gene product thereof. In some embodiments, an antisense oligonucleotide is capable of directing a decrease in the level, expression or activity of the target nucleic acid or a gene product thereof, via a mechanism that involves RNaseH, steric hindrance and/or RNA interference.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). In some embodiments, use of the term “about” in reference to dosages means±5 mg/kg/day.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Characteristic portion: As used herein, the phrase a “characteristic portion” of a protein or polypeptide is one that contains a continuous stretch of amino acids, or a collection of continuous stretches of amino acids, that together are characteristic of a protein or polypeptide. Each such continuous stretch generally will contain at least two amino acids. In general, a characteristic portion is one that, in addition to the sequence identity specified above, shares at least one functional characteristic with the relevant intact protein.

Characteristic structural element: The term “characteristic structural element” or “structural element” refers to a distinctive structural element that is found in all members of a family of polypeptides, small molecules, or nucleic acids, and therefore can be used by those of ordinary skill in the art to define members of the family. In some embodiments, a structural element of a single-stranded RNAi agent includes, but is not limited to: a 5′-end structure, a 5′-end region, a 5′ nucleotide moiety, a seed region, a post-seed region, a 3′-end region, a 3′-terminal dinucleotide, a 3′ cap, a pattern of modifications, a pattern of stereochemistry in the backbone, additional chemical moieties, etc.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted form thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is oxygen, sulfur, nitrogen, phosphorus, or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.).

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5-to 7-membered monocyclic or 7-to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Lower alkyl: The term “lower alkyl” refers to a C₁₋₄ straight or branched alkyl group. Example lower alkyl groups are methyl, ethyl, propyl, isopropyl, butyl, isobutyl, and tert-butyl.

Lower haloalkyl: The term “lower haloalkyl” refers to a C₁₋₄ straight or branched alkyl group that is substituted with one or more halogen atoms.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable atom, e.g., a suitable carbon atom, are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR, —SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯}S(O)₂NR^{◯}₂; —N(R^{◯}S(O)₂R^{◯}; —N(OR^{◯})R^{◯}; —C(NH)NR^{◯}₂; —Si(R^{◯})₃; —OSi(R^{◯})₃; —B(R^{◯})₂; —OB(R^{◯})₂; —OB(OR^{◯})₂; —P(R^{◯})₂; —P(OR^{◯})₂; —OP(R^{◯})₂; —OP(OR^{◯})₂; —P(O)(R^{◯})₂; —P(O)(OR^{◯})₂; —OP(O)(R^{◯})₂; —OP(O)(OR^{◯})₂; —OP(O)(OR^{◯})(SR^{◯}; —SP(O)(R^{◯})₂; —SP(O)(OR^{◯})₂; —N(R^{◯}P(O)(R^{◯})₂; —N(R^{◯})P(O)(OR^{◯})₂; —P(R^{◯})₂[B(R^{◯)}₃]; —P(OR^{◯})₂[B(R^{◯})₃]; —OP(R^{◯})₂[B(R^{◯})₃]; —OP(OR^{◯})₂[B(R^{◯})₃]; —(C₁₋₄ straight or branched)alkylene)O—N(R^{◯})₂; or —(C₁₋₄ straight or branched)alkylene)C(O)O—N(R^{◯})₂, wherein each R^{◯} may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{◯}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}₃, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)_{0_1}Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

RNA interference: As used herein, the terms “RNA interference” or “RNAi” refer to a post-transcriptional, targeted gene-silencing process involving the RISC (RNA-induced silencing complex). A process of RNAi reportedly naturally occurs when ribonuclease III (Dicer) cleaves a longer dsRNA into shorter fragments called siRNAs. A naturally-produced siRNA (small interfering RNA) is typically about 21 to 23 nucleotides long with an about 19 basepair duplex and two single-stranded overhangs and is typically RNA. These RNA segments then reportedly direct the degradation of the target nucleic acid, such as a mRNA or pre-mRNA. Dicer has reportedly also been implicated in the excision of 21- and 22-nucleotide small temporal RNAs (stRNAs) from precursor RNA of conserved structure that are implicated in translational control. Hutvagner et al. 2001, Science, 293, 834. Those skilled in the art are aware that RNAi can be mediated by a single-stranded or a double-stranded oligonucleotide that includes a sequence complementary or substantially complementary to a target sequence (e.g., in a target mRNA). Thus, in some embodiments of the present disclosure, a single-stranded oligonucleotide as described herein may act as an RNAi agent; in some embodiments, a double-stranded oligonucleotide as described herein may act as an RNAi agent. In some embodiments, an RNAi response involves an endonuclease complex, commonly referred to as an RNA-induced silencing complex (RISC), which directs cleavage of single-stranded mRNA complementary to the antisense strand of the siRNA. In some embodiments, RISC directs cleavage of target RNA complementary to provided oligonucleotides which can function as single-stranded RNAi agent. In some embodiments, cleavage of a target RNA takes place in the middle of the region complementary to the antisense strand of a siRNA duplex or single-stranded RNAi agent. In some embodiments, RNA interference is directed by a single-stranded oligonucleotide which acts as a single-stranded RNAi agent that can direct RNA interference in a mechanism involving the RISC pathway.

RNAi agent: As used herein, the term “RNAi agent,” “iRNA agent”, and the like, refer to an APOC3 oligonucleotide that, when administered to a system in which a target gene product (e.g., a transcript, such as a pre-mRNA or a mRNA, of a target gene) is being or has been expressed, reduces level and/or activity (e.g., translation) of that target gene product. In some embodiments, an RNAi agent may be or comprise a single-stranded oligonucleotide or a double-stranded oligonucleotide. In some embodiments, an RNAi agent may have a structure recognized in the art as a siRNA (short inhibitory RNA), shRNA (short or small hairpin RNA), dsRNA (double-stranded RNA), microRNA, etc. In some embodiments, an RNAi agent may specifically bind to a RNA target (e.g., a transcript of a target gene). In some embodiments, upon binding to its target, and RNAi agent is loaded to the RISC (RNA-induced silencing complex). In some embodiments, an RNAi agent directs degradation of, and/or inhibits translation of, its target, in some embodiments via a mechanism involving the RISC (RNA-induced silencing complex) pathway. In some embodiments, an RNAi agent is an APOC3 oligonucleotide that activates the RISC complex/pathway. In some embodiments, an RNAi agent comprises an antisense strand sequence. In some embodiments, an RNAi agent includes only one oligonucleotide strand (e.g., is a single-stranded oligonucleotide). In some embodiments, a single-stranded RNAi agent oligonucleotide can be or comprise a sense or antisense strand sequence, as described by Sioud 2005 J. Mol. Biol. 348: 1079-1090. In some embodiments, a RNAi agent is a compound capable of directing RNA interference. In some embodiments, a RNAi agent may have a structure or format as is found in “canonical” siRNA structure). In some embodiments, an RNAi agent may have a structure that differs from a “canonical” siRNA structure. To give but a few examples, in some embodiments, an RNAi agent can be longer or shorter than the canonical, can be blunt-ended, and/or can comprise one or more modifications, mismatches, gaps and/or nucleotide replacements. In some embodiments, an RNAi agent contains a 3′-end cap as described in the present disclosure. Without wishing to be bound by any particular theory, Applicant proposes that, in some embodiments, a 3′-end cap can allow both of two functions: (1) allowing RNA interference; and (2) increasing duration of activity and/or biological half-life, which may be accomplished, for example, by increased binding to the PAZ domain of Dicer and/or one or more Ago proteins and/or reducing or preventing degradation of the RNAi agent (e.g., by nucleases such as those in the serum or intestinal fluid). In some embodiments, a RNAi agent of the present disclosure targets (e.g., binds to, anneals to, etc.) a target mRNA. In some embodiments, exposure of a RNAi agent to its target results in a decrease of activity, level and/or expression, e.g., a “knock-down” or “knock-out” of the target. Particularly, in some embodiments, in the case of a disease, disorder and/or condition characterized by over-expression and/or hyper-activity of a target gene, administration of a RNAi agent to a cell, tissue, or subject knocks down the target gene enough to restore a normal level of activity, or to reduce activity to a level that can alleviate, ameliorate, relieve, inhibit, prevent, delay onset of, reduce severity of, and/or reduce incidence of one or more symptoms or features of a disease, disorder, and/or condition of the disease, disorder, and/or condition. In some embodiments, a RNAi agent is double-stranded comprising an antisense strand which is a single-stranded RNAi agent as described herein, which, in combination with a sense strand, can direct RNA interference.

Single-stranded RNA interference: As used herein, the phrases “single-stranded RNAi” or “single-stranded RNA interference” or the like refer to a process or method of gene silencing directed at least in part by administration of a single-stranded RNAi agent to a system (e.g., cells, tissues, organs, subjects, etc.) where RNAi is to be directed by the agent, and which requires the RISC pathway. The terms may be utilized herein in certain instances to distinguish from “double-stranded RNAi” or “double-stranded RNA interference”, in which a double-stranded RNAi agent is administered to a system, and may be further processed, for example so that one of its two strands is loaded to RISC to, e.g., suppress translation, cleave target RNA, etc.

Single-stranded RNAi agent: As used herein, the phrase “single-stranded RNAi agent” refers to a single-stranded oligonucleotide that can direct single-stranded RNA interference (RNAi or iRNA) or gene silencing via the RISC pathway. A single-stranded RNAi agent can comprise a polymer of one or more single-stranded nucleotides.

Subject: As used herein, the term “subject” or “test subject” refers to any organism to which a provided compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially complementary to a second sequence is not identical to the second sequence, but is mostly or nearly identical to the second sequence. In addition, one of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder and/or condition has been diagnosed with and/or displays one or more symptoms of a disease, disorder and/or condition.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Systemic: The phrases “systemic administration,” “administered systemically,” “peripheral administration,” and “administered peripherally” as used herein have their art-understood meaning referring to administration of a compound or composition such that it enters the recipient's system.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc) state or context. Those of ordinary skill in the art will appreciate that wild type genes and polypeptides often exist in multiple different forms (e.g., alleles).

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotide linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleotide: The term “nucleotide” as used herein refers to a monomeric unit of a polynucleotide that consists of a heterocyclic base, a sugar, and one or more internucleotidic linkages. The naturally occurring bases (guanine, (G), adenine, (A), cytosine, (C), thymine, (T), and uracil (U)) are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Analog: The term “analog” means any functional analog wherein a chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Nucleoside: The term “nucleoside” refers to a moiety wherein a nucleobase or a modified nucleobase is covalently bound to a sugar or modified sugar.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Nucleoside analog: The term “nucleoside analog” refers to a chemical moiety which is chemically distinct from a natural nucleoside, but which is capable of performing at least one function of a nucleoside. In some embodiments, a nucleoside analog comprises an analog of a sugar and/or an analog of a nucleobase. In some embodiments, a modified nucleoside is capable of at least one function of a nucleoside, e.g., forming a moiety in a polymer capable of base-pairing to a nucleic acid comprising a complementary sequence of bases.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars.

Modified sugar: The term “modified sugar” refers to a moiety that can replace a sugar. A modified sugar mimics the spatial arrangement, electronic properties, or some other physicochemical property of a sugar.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, the naturally-occurring nucleobases are modified adenine, guanine, uracil, cytosine, or thymine. In some embodiments, the naturally-occurring nucleobases are methylated adenine, guanine, uracil, cytosine, or thymine. In some embodiments, a nucleobase is a “modified nucleobase,” e.g., a nucleobase other than adenine (A), guanine (G), uracil (U), cytosine (C), and thymine (T). In some embodiments, the modified nucleobases are methylated adenine, guanine, uracil, cytosine, or thymine. In some embodiments, the modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs.

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases.

3′-end cap: The term “3′-end cap” refers to a non-nucleotidic chemical moiety bound to the 3′-end of an APOC3 oligonucleotide, e.g., a RNAi agent. In some embodiments, a 3′-end cap replaces a 3′-terminal dinucleotide. In some embodiments, a 3′-end cap of an APOC3 oligonucleotide performs at least one of the following functions: allowing RNA interference directed by the oligonucleotide, protecting the oligonucleotide from degradation or reducing the amount or rate of degradation of the oligonucleotide (e.g., by nucleases), reducing the off-target effects of a sense strand, or increasing the activity, duration or efficacy of RNA interference directed by the oligonucleotide. By describing a 3′-end cap as “non-nucleotidic”, it is meant that a 3′-end cap is not a nucleotidic moiety, or oligonucleotide moiety, connected to a sugar moiety of the rest of an APOC3 oligonucleotide as it would do if it is part of an APOC3 oligonucleotide chain. Certain example 3′-end caps are described herein. A person having ordinary skill understands that others 3′-end caps known in the art can be utilized in accordance in the present disclosure.

Blocking group: The term “blocking group” refers to a group that masks the reactivity of a functional group. The functional group can be subsequently unmasked by removal of the blocking group. In some embodiments, a blocking group is a protecting group.

Moiety: The term “moiety” refers to a specific segment or functional group of a molecule. Chemical moieties are often recognized chemical entities embedded in or appended to a molecule.

Solid support: The term “solid support” refers to any support which enables synthesis of nucleic acids. In some embodiments, the term refers to a glass or a polymer, that is insoluble in the media employed in the reaction steps performed to synthesize nucleic acids, and is derivatized to comprise reactive groups. In some embodiments, the solid support is Highly Cross-linked Polystyrene (HCP) or Controlled Pore Glass (CPG). In some embodiments, the solid support is Controlled Pore Glass (CPG). In some embodiments, the solid support is hybrid support of Controlled Pore Glass (CPG) and Highly Cross-linked Polystyrene (HCP).

Linker or Linking moiety: The terms “linker”, “linking moiety” and the like refer to any chemical moiety which connects one chemical moiety to another. In some embodiments, a linker is a moiety which connects one oligonucleotide to another oligonucleotide in a multimer. In some embodiments, a linker is a moiety optionally positioned between the terminal nucleoside and the solid support or between the terminal nucleoside and another nucleoside, nucleotide, or nucleic acid.

Gene: The terms “gene,” “recombinant gene” and “gene construct” as used herein, refer to a DNA molecule, or portion of a DNA molecule, that encodes a protein or a portion thereof. The DNA molecule can contain an open reading frame encoding the protein (as exon sequences) and can further include intron sequences. The term “intron” as used herein, refers to a DNA sequence present in a given gene which is not translated into protein and is found in some, but not all cases, between exons. It can be desirable for the gene to be operably linked to, (or it can comprise), one or more promoters, enhancers, repressors and/or other regulatory sequences to modulate the activity or expression of the gene, as is well known in the art.

Complementary DNA: As used herein, a “complementary DNA” or “CDNA” includes recombinant polynucleotides synthesized by reverse transcription of mRNA and from which intervening sequences (introns) have been removed.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. As used herein, the term “oligonucleotide strand” encompasses a single-stranded oligonucleotide. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. In some embodiments, oligonucleotides are capable of directing a decrease in the expression and/or level of a target gene or its gene product. In some embodiments, oligonucleotides are capable of directing a decrease in the expression and/or level of a target gene or its gene product via RNA interference. In some embodiments, oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, oligonucleotides are capable of directing a decrease in the expression and/or level of a target gene or its gene product via RNA interference and/or RNase H-mediated knockdown. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Double-stranded and single-stranded oligonucleotides that are effective in inducing RNA interference are also referred to as a RNAi agent or iRNA agent, herein. In some embodiments, these RNA interference inducing oligonucleotides associate with a cytoplasmic multi-protein complex known as RNAi-induced silencing complex (RISC). In many embodiments, double-stranded RNAi agents are sufficiently long that they can be cleaved by an endogenous molecule, e.g., by Dicer, to produce smaller oligonucleotides that can enter the RISC machinery and participate in RISC mediated cleavage and/or translation suppression of a target sequence, e.g. a target mRNA sequence.

Oligonucleotides of the present disclosure can be of various lengths. In particular embodiments, oligonucleotides can range from about 2 to about 200 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, or from about 20 to about 30 nucleotides in length. In some embodiments, an APOC3 oligonucleotide is from about 10 to about 40 nucleotides in length. In some embodiments, an APOC3 oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length. In some embodiments, each nucleotide counted in a length independently comprises an optionally substituted nucleobase selected from adenine, cytosine, guanosine, thymine, and uracil.

Internucleotidic linkage: As used herein, the phrase “internucleotidic linkage” refers generally to a linkage linking nucleoside units of an APOC3 oligonucleotide or a nucleic acid. In some embodiments, an internucleotidic linkage is a phosphodiester linkage, as found in naturally occurring DNA and RNA molecules (natural phosphate linkage). In some embodiments, the term “internucleotidic linkage” includes a modified internucleotidic linkage. In some embodiments, an internucleotidic linkage is a “modified internucleotidic linkage” wherein each oxygen atom of the phosphodiester linkage is optionally and independently replaced by an organic or inorganic moiety. In some embodiments, such an organic or inorganic moiety is selected from but not limited to ═S, ═Se, ═NR′, —SR′, —SeR′, —N(R′)₂, B(R′)₃, —S—, —Se—, and —N(R′)—, wherein each R′ is independently as defined and described in the present disclosure. In some embodiments, an internucleotidic linkage is a phosphotriester linkage, phosphorothioate diester linkage

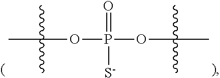 [SMILES: *OP(=O)([S-])O*]

or modified phosphorothioate triester linkage.

It is understood by a person of ordinary skill in the art that an internucleotidic linkage may exist as an anion or cation at a given pH due to the existence of acid or base moieties in the linkage.

In some embodiments, “All-(Rp)” or “All-(Sp)” is used to indicate that all chiral linkage phosphorus atoms in oligonucleotide have the same Rp or Sp configuration, respectively.

Oligonucleotide type: As used herein, the phrase “oligonucleotide type” is used to define an APOC3 oligonucleotide that has a particular base sequence, pattern of backbone linkages (i.e., pattern of internucleotidic linkage types, for example, phosphate, phosphorothioate, etc.), pattern of backbone chiral centers (i.e. pattern of linkage phosphorus stereochemistry (Rp/Sp)), and pattern of backbone phosphorus modifications (e.g., pattern of “—XLR¹” groups in formula I). In some embodiments, oligonucleotides of a common designated “type” are structurally identical to one another.

One of skill in the art will appreciate that synthetic methods of the present disclosure provide for a degree of control during the synthesis of an APOC3 oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an APOC3 oligonucleotide strand is designed and/or selected in advance to have a particular combination of stereocenters at the linkage phosphorus. In some embodiments, an APOC3 oligonucleotide strand is designed and/or determined to have a particular combination of modifications at the linkage phosphorus. In some embodiments, an APOC3 oligonucleotide strand is designed and/or selected to have a particular combination of bases. In some embodiments, an APOC3 oligonucleotide strand is designed and/or selected to have a particular combination of one or more of the above structural characteristics. In some embodiments, the present disclosure provides compositions comprising or consisting of a plurality of oligonucleotide molecules (e.g., chirally controlled oligonucleotide compositions). In some embodiments, all such molecules are of the same type (i.e., are structurally identical to one another). In many embodiments, however, provided compositions comprise a plurality of oligonucleotides of different types, typically in pre-determined relative amounts.

Chiral control: As used herein, “chiral control” refers to control of the stereochemical designation of a chiral linkage phosphorus in a chiral internucleotidic linkage within an APOC3 oligonucleotide. In some embodiments, a control is achieved through a chiral element that is absent from the sugar and base moieties of an APOC3 oligonucleotide, for example, in some embodiments, a control is achieved through use of one or more chiral auxiliaries during oligonucleotide preparation as exemplified in the present disclosure, which chiral auxiliaries often are part of chiral phosphoramidites used during oligonucleotide preparation. In contrast to chiral control, a person having ordinary skill in the art appreciates that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in a chiral internucleotidic linkage within an APOC3 oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share 1) a common base sequence, 2) a common pattern of backbone linkages, and 3) a common pattern of backbone phosphorus modifications, wherein the plurality of oligonucleotides (or nucleic acids) share the same stereochemistry at one or more chiral internucleotidic linkages (chirally controlled internucleotidic linkages), and the level of the plurality of oligonucleotides (or nucleic acids) in the composition is pre-determined (e.g., through chirally controlled oligonucleotide preparation to form one or more chiral internucleotidic linkages). In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a predetermined level is be about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 50%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an APOC3 oligonucleotide type), or of all oligonucleotides in a composition that share a common base sequence, a common pattern of backbone linkages, and a common pattern of backbone phosphorus modifications are oligonucleotides of the plurality, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 1%400% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 350%, 40%, 45%, 50%, 55%, 60%, 65%, 70%, 75%, 80%, 85%, 90%, 95%, or 99%) of chiral internucleotidic linkages. In some embodiments, each chiral internucleotidic linkage is a chiral controlled internucleotidic linkage, and the composition is a completely chirally controlled oligonucleotide composition. In some embodiments, not all chiral internucleotidic linkages are chiral controlled internucleotidic linkages, and the composition is a partially chirally controlled oligonucleotide composition. In some embodiments, a chirally controlled oligonucleotide composition comprises predetermined levels of individual oligonucleotide or nucleic acids types. For instance, in some embodiments a chirally controlled oligonucleotide composition comprises one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of a oligonucleotide type, which composition comprises a predetermined level of a plurality of oligonucleotides of the oligonucleotide type.

Chirally pure: as used herein, the phrase “chirally pure” is used to describe the relative amount of an APOC3 oligonucleotide, e.g., a single-stranded RNAi agent, in which all of the oligonucleotides exist in a single diastereomeric form with respect to the linkage phosphorus.

Chirally uniform: as used herein, the phrase “chirally uniform” is used to describe an APOC3 oligonucleotide molecule or type in which all nucleotide units have the same stereochemistry at the linkage phosphorus. For instance, an APOC3 oligonucleotide whose nucleotide units all have Rp stereochemistry at the linkage phosphorus is chirally uniform. Likewise, an APOC3 oligonucleotide whose nucleotide units all have Sp stereochemistry at the linkage phosphorus is chirally uniform.

Predetermined: By predetermined (or pre-determined) is meant deliberately selected, for example as opposed to randomly occurring or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that are not controlled to intentionally generate the particular chemistry and/or stereochemistry features is not a “predetermined” composition. In some embodiments, a predetermined composition is one that can be intentionally reproduced (e.g., through repetition of a controlled process). In some embodiments, a predetermined level of a plurality of oligonucleotides in a composition means that the absolute amount, and/or the relative amount (ratio, percentage, etc.) of the plurality of oligonucleotides in the composition is controlled. In some embodiments, a predetermined level of a plurality of oligonucleotides in a composition is achieved through chirally controlled oligonucleotide preparation.

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester of an internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is P^{L} of Formula I. In some embodiments, a linkage phosphorus atom is chiral.

P-modification: as used herein, the term “P-modification” refers to any modification at the linkage phosphorus other than a stereochemical modification. In some embodiments, a P-modification comprises addition, substitution, or removal of a pendant moiety covalently attached to a linkage phosphorus. In some embodiments, the “P-modification” is —X-L-R¹ wherein each of X, L and R¹ is independently as defined and described in the present disclosure.

For purposes of this disclosure, the chemical elements are identified in accordance with the Periodic Table of the Elements, CAS version, Handbook of Chemistry and Physics, 67th Ed., 1986-87, inside cover.

The methods and structures described herein relating to compounds and compositions of the disclosure also apply to the pharmaceutically acceptable acid or base addition salts and all stereoisomeric forms of these compounds and compositions.

### BRIEF DESCRIPTION OF THE DRAWINGS

FIG. 1. FIG. 1, including FIG. 1A to 1L, presents cartoons of various ssRNAi formats and hybrid formats.

FIG. 2. FIG. 2 presents cartoons of various antisense oligonucleotide formats.

FIG. 3. FIG. 3A shows example multimer formats. Oligonucleotides can be joined directly and/or through linkers. As illustrated, a multimer can comprise oligonucleotide monomers of the same or different structures/types. In some embodiments, a monomer of a multimer is an ssRNAi agent. In some embodiments, a monomer of a multimer is a RNase H-dependent antisense oligonucleotide (ASO). Monomers can be joined through various positions, for example, the 5′-end, the 3′-end, or positions in between. FIG. 3B shows example chemistry approaches for joining monomers, which monomers may perform their functions through various pathways, to form multimers.

### DETAILED DESCRIPTION OF CERTAIN EMBODIMENTS

Synthetic oligonucleotides provide useful molecular tools in a wide variety of applications. For example, oligonucleotides are useful in therapeutic, diagnostic, research, and new nanomaterials applications. The use of naturally occurring nucleic acids (e.g., unmodified DNA or RNA) is limited, for example, by their susceptibility to endo- and exo-nucleases. As such, various synthetic counterparts have been developed to circumvent these shortcomings. These include synthetic oligonucleotides that contain chemical modifications, e.g., base modifications, sugar modifications, backbone modifications, etc., which, among other things, render these molecules less susceptible to degradation and improve other properties of oligonucleotides. From a structural point of view, modifications to internucleotide phosphate linkages can introduce chirality, and certain properties of oligonucleotides may be affected by configurations of phosphorus atoms that form the backbone of oligonucleotides. For example, in vitro studies have shown that properties of antisense oligonucleotides, such as binding affinity, sequence specific binding to complementary RNA, stability to nucleases, are affected by, inter alia, chirality of backbone phosphorus atoms.

Among other things, the present disclosure encompasses the recognition that structural elements of oligonucleotides, such as chemical modifications (e.g., modifications of sugar, base, and/or internucleotidic linkages) or patterns thereof, conjugation to lipids or other moieties, and/or stereochemistry [e.g., stereochemistry of backbone chiral centers (chiral internucleotidic linkages), and/or patterns thereof], can have significant impact on properties and activities (e.g., stability, specificity, selectivity, activities to reduce levels of products (transcripts and/or protein) of target genes, etc.). In some embodiments, oligonucleotide properties can be adjusted by optimizing chemical modifications (modifications of base, sugar, and/or internucleotidic linkage moieties), patterns of chemical modifications, stereochemistry and/or patterns of stereochemistry.

In some embodiments, the present disclosure demonstrates that oligonucleotide compositions comprising oligonucleotides with controlled structural elements, e.g., controlled chemical modifications and/or controlled backbone stereochemistry patterns, provide unexpected properties and activities, including but not limited to those described herein. In some embodiments, provided compositions comprising oligonucleotides having chemical modifications (e.g., base modifications, sugar modification, internucleotidic linkage modifications, etc.) or patterns thereof have improved properties and activities. Non-limiting examples of such improved properties include: directing a decrease in the expression and/or level of a target gene or its gene product; and/or directing RNA interference; and/or directing RNase H-mediated knockdown. In some embodiments, the present disclosure provides technologies (e.g., oligonucleotides, compositions, methods, etc.) for single-stranded RNAi. In some embodiments, a provided oligonucleotide is a ssRNAi agent.

In some embodiments, RNA interference is reportedly a post-transcriptional, targeted gene-silencing technique that uses an RNAi agent to target a RNA, e.g., a gene transcript such as a messenger RNA (mRNA), comprising a sequence complementary to the RNAi agent, for cleavage mediated by the RISC (RNA-induced silencing complex) pathway. In nature, a type of RNAi reportedly occurs when ribonuclease III (Dicer) cleaves a long dsRNA (double-stranded RNA) (e.g., a foreign dsRNA introduced into a mammalian cell) into shorter fragments called siRNAs. siRNAs (small interfering RNAs or short inhibitory RNAs) are typically about 21 to 23 nucleotides long and comprise about 19 base pair duplexes. The smaller RNA segments then reportedly mediate the degradation of the target mRNA. The RNAi response also reportedly features an endonuclease complex, commonly referred to as an RNA-induced silencing complex (RISC), which directs cleavage of single-stranded mRNA complementary to the antisense strand of the siRNA. Cleavage of the target RNA reportedly takes place in the middle of the region complementary to the antisense strand of the siRNA duplex. The use of the RNAi agent to a target transcript reportedly results in a decrease of gene activity, level and/or expression, e.g., a “knock-down” or “knock-out” of the target gene or target sequence. Artificial siRNAs are useful both as therapeutics and for experimental use.

In one aspect, an RNA interference agent includes a single stranded RNA that interacts with a target RNA sequence to direct the cleavage of the target RNA. Without wishing to be bound by theory, long double stranded RNA introduced into plants and invertebrate cells is reportedly broken down into siRNA by a Type III endonuclease known as Dicer (Sharp et al., Genes Dev. 2001, 15:485). Dicer, a ribonuclease-III-like enzyme, reportedly processes the dsRNA into 19-23 base pair short interfering RNAs with characteristic two base 3′ overhangs (Bernstein, et al., (2001) Nature 409:363). The siRNAs are reportedly then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15: 188). Thus, in one aspect the disclosure relates to a single stranded RNA that promotes the formation of a RISC complex to effect silencing of a target gene.

In some embodiments, a suitable RNAi agent can be selected by any processes known in the art or conceivable by one of ordinary skill in the art in accordance with the present disclosure. For example, the selection criteria can include one or more of the following steps: initial analysis of the target gene sequence and design of RNAi agents; this design can take into consideration sequence similarity across species (human, cynomolgus, mouse, etc.) and dissimilarity to other (non-target) genes; screening of RNAi agents in vitro (e.g., at 10 nM in cells expressing the target transcript); determination of EC50 or IC50 in cells; determination of viability of cells treated with RNAi agents, wherein it is desired, in some embodiments, that the RNAi agent to the target not inhibit the viability of these cells; testing with human PBMC (peripheral blood mononuclear cells), e.g., to test levels of TNF-alpha to estimate immunogenicity, wherein immunostimulatory sequences are usually less desired; testing in human whole blood assay, wherein fresh human blood is treated with an RNAi agent and cytokine/chemokine levels are determined [e.g., TNF-alpha (tumor necrosis factor-alpha) and/or MCP1 (monocyte chemotactic protein 1)], wherein immunostimulatory sequences are usually less desired; determination of gene knockdown in vivo using cells or tumors in test animals; and optimization of specific modifications of the RNAi agents.

The so-called canonical siRNA structure is reportedly a double-stranded RNA molecule, wherein each strand is about 21 nucleotides long. The two strands are reportedly an antisense (or “guide”) strand, which recognizes and binds to a complementary sequence in the target transcript, and a sense (or “passenger”) strand, which is complementary to the antisense strand. The sense and antisense strands are reportedly largely complementary, typically forming two 3′ overhangs of 2 nucleotides on both ends.

While a canonical siRNA structure is reportedly double-stranded, RNAi agent can also be single-stranded. In some embodiments, a single-stranded RNAi agent corresponds to an antisense strand of a double-stranded siRNA, and the single-stranded RNAi agent lacks a corresponding passenger strand.

However, it has been reported that not all tested structural elements for single-stranded RNAi agents are effective; introduction of some structural elements into an APOC3 oligonucleotide can reportedly interference with single-stranded RNA interference activity.

In some embodiments, the present disclosure provides oligonucleotides and compositions useful as RNAi agent. In some embodiments, the present disclosure provides oligonucleotides and compositions useful as single-stranded RNAi agent. The present disclosure, among other things, provides novel structures of single-stranded oligonucleotides capable of directing RNA interference. Without wishing to be bound by any particular theory, this disclosure notes that single-stranded RNAi agents have advantages over double-stranded RNAi agents. For example, single-stranded RNAi agents have a lower cost of goods, as the construction of only one strand is required. Additionally or alternatively, only one strand (the antisense strand) is administered to target a target transcript. A source of off-target effects directed by dsRNA is loading of the sense strand into RISC and binding to and knockdown of undesired targets (Jackson et al. 2003 Nat. Biotech. 21: 635-637), a single-stranded RNAi agent can elicit fewer off-target effects than a corresponding double-stranded RNAi agent. In addition, some single-stranded RNAi agents, including some disclosed herein, can target particular sequences which have not previously been successfully targeted with double-stranded RNAi agents (for example, they can reduce levels of the sequences, and/or products (transcripts and/or proteins) of the sequences, significantly more than double-stranded RNAi agents). The present disclosure, among other things, provides novel formats (modifications, stereochemistry, combinations thereof, etc.) for oligonucleotides which can direct single-stranded RNA interference.

### Oligonucleotides

In some embodiments, provided oligonucleotides can direct a decrease in the expression and/or level of a target gene or its gene product. In some embodiments, provided oligonucleotides can direct a decrease in levels of target products. In some embodiments, provided oligonucleotide can reduce levels of transcripts of target genes. In some embodiments, provided oligonucleotide can reduce levels of mRNA of target genes. In some embodiments, provided oligonucleotide can reduce levels of proteins encoded by target genes. In some embodiments, provided oligonucleotides can direct a decrease in the expression and/or level of a target gene or its gene product via RNA interference. In some embodiments, provided oligonucleotides can direct a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides can direct a decrease in the expression and/or level of a target gene or its gene product via RNA interference and/or RNase H-mediated knockdown. In some embodiments, provided oligonucleotides can direct a decrease in the expression and/or level of a target gene or its gene product by sterically blocking translation after binding to a target gene mRNA, and/or by altering or interfering with mRNA splicing and/or exon inclusion or exclusion. In some embodiments, provided oligonucleotides comprise one or more structural elements described herein or known in the art in accordance with the present disclosure, e.g., base sequences; modifications; stereochemistry; patterns of internucleotidic linkages; patterns of backbone linkages; patterns of backbone chiral centers; patterns of backbone phosphorus modifications; additional chemical moieties, including but not limited to, one or more targeting moieties, lipid moieties, and/or carbohydrate moieties, etc.; seed regions; post-seed regions; 5′-end structures; 5′-end regions; 5′ nucleotide moieties; 3′-end regions; 3′-terminal dinucleotides; 3′-end caps; etc. In some embodiments, a seed region of an APOC3 oligonucleotide is or comprises the second to eighth, second to seventh, second to sixth, third to eighth, third to seventh, third to seven, or fourth to eighth or fourth to seventh nucleotides, counting from the 5′ end; and the post-seed region of the oligonucleotide is the region immediately 3′ to the seed region, and interposed between the seed region and the 3′ end region.

In some embodiments, a provided composition comprises an APOC3 oligonucleotide. In some embodiments, a provided composition comprises one or more lipid moieties, one or more carbohydrate moieties (unless otherwise specified, other than sugar moieties of nucleoside units that form oligonucleotide chain with internucleotidic linkages), and/or one or more targeting components.

In some embodiments, a target sequence is a sequence to which an APOC3 oligonucleotide as described herein binds. In many embodiments, a target sequence is identical to, or is an exact complement of, a sequence of a provided oligonucleotide, or of consecutive residues therein (e.g., a provided oligonucleotide includes a target-binding sequence that is identical to, or an exact complement of, a target sequence). In some embodiments, a small number of differences/mismatches is tolerated between (a relevant portion of) an APOC3 oligonucleotide and its target sequence. In many embodiments, a target sequence is present within a target gene. In many embodiments, a target sequence is present within a transcript (e.g., an mRNA and/or a pre-mRNA) produced from a target gene.

Various linker, lipid moieties, carbohydrate moieties and targeting moieties, including many known in the art, can be utilized in accordance with the present disclosure. In some embodiments, a lipid moiety is a targeting moiety. In some embodiments, a carbohydrate moiety is a targeting moiety. In some embodiments, a targeting moiety is a lipid moiety. In some embodiments, a targeting moiety is a carbohydrate moiety. As readily appreciated by those skilled in the art, various linkers, including those described in the present disclosure, can be utilized in accordance with the present disclosure to link two moieties, for example, a lipid/carbohydrate/targeting component with an APOC3 oligonucleotide moiety. As readily appreciated by those skilled in the art, linkers described for linking two moieties can also be used to link other moieties, for example, linkers for linking a lipid and an APOC3 oligonucleotide moiety can also be used to link a carbohydrate or target moiety with an APOC3 oligonucleotide moiety and vice versa.

In some embodiments, the present disclosure provides oligonucleotides and oligonucleotide compositions that are chirally controlled. For instance, in some embodiments, a provided composition contains predetermined levels of one or more individual oligonucleotide types, wherein an APOC3 oligonucleotide type is defined by: 1) base sequence; 2) pattern of backbone linkages; 3) pattern of backbone chiral centers; and 4) pattern of backbone P-modifications. In some embodiments, a particular oligonucleotide type may be defined by 1A) base identity; 1B) pattern of base modification; 1C) pattern of sugar modification; 2) pattern of backbone linkages; 3) pattern of backbone chiral centers; and 4) pattern of backbone P-modifications. In some embodiments, oligonucleotides of the same oligonucleotide type are identical. In some embodiments, the present disclosure provides chirally controlled oligonucleotide compositions of oligonucleotides, wherein the composition comprises a predetermined level of a plurality of oligonucleotides, wherein oligonucleotides of the plurality share a common base sequence, and comprise the same configuration of linkage phosphorus at at least 1, 2, 3, 4, 5, 6, 7, 8, 9, 10, 11, 12, 13, 14, 15, 16, 17, 18, 19, 20, 21, 22, 23, 24, or 25 chiral internucleotidic linkages (chirally controlled internucleotidic linkages).

In some embodiments, provided oligonucleotides comprise 2-30 chirally controlled internucleotidic linkages. In some embodiments, provided oligonucleotides comprise 5-30 chirally controlled internucleotidic linkages. In some embodiments, provided oligonucleotides comprise 10-30 chirally controlled internucleotidic linkages. In some embodiments, provided oligonucleotides comprise 1 chirally controlled internucleotidic linkage. In some embodiments, provided oligonucleotides comprise 2 chirally controlled internucleotidic linkages. In some embodiments, provided oligonucleotides comprise 3 chirally controlled internucleotidic linkages. In some embodiments, provided oligonucleotides comprise 4 chirally controlled internucleotidic linkages. In some embodiments, provided oligonucleotides comprise 5 chirally controlled internucleotidic linkages. In some embodiments, provided oligonucleotides comprise 6 chirally controlled internucleotidic linkages. In some embodiments, provided oligonucleotides comprise 7 chirally controlled internucleotidic linkages. In some embodiments, provided oligonucleotides comprise 8 chirally controlled internucleotidic linkages. In some embodiments, provided oligonucleotides comprise 9 chirally controlled internucleotidic linkages. In some embodiments, provided oligonucleotides comprise 10 chirally controlled internucleotidic linkages. In some embodiments, provided oligonucleotides comprise 11 chirally controlled internucleotidic linkages. In some embodiments, provided oligonucleotides comprise 12 chirally controlled internucleotidic linkages. In some embodiments, provided oligonucleotides comprise 13 chirally controlled internucleotidic linkages. In some embodiments, provided oligonucleotides comprise 14 chirally controlled internucleotidic linkages. In some embodiments, provided oligonucleotides have 15 chirally controlled internucleotidic linkages. In some embodiments, provided oligonucleotides have 16 chirally controlled internucleotidic linkages. In some embodiments, provided oligonucleotides have 17 chirally controlled internucleotidic linkages. In some embodiments, provided oligonucleotides have 18 chirally controlled internucleotidic linkages. In some embodiments, provided oligonucleotides have 19 chirally controlled internucleotidic linkages. In some embodiments, provided oligonucleotides have 20 chirally controlled internucleotidic linkages.

In some embodiments, a provided oligonucleotide is a unimer. In some embodiments, a provided oligonucleotide is a P-modification unimer. In some embodiments, a provided oligonucleotide is a stereounimer. In some embodiments, a provided oligonucleotide is a stereounimer of configuration Rp. In some embodiments, a provided oligonucleotide is a stereounimer of configuration Sp.

In some embodiments, a provided oligonucleotide is an altmer. In some embodiments, a provided oligonucleotide is a P-modification altmer. In some embodiments, a provided oligonucleotide is a stereoaltmer.

In some embodiments, a provided oligonucleotide is a blockmer. In some embodiments, a provided oligonucleotide is a P-modification blockmer. In some embodiments, a provided oligonucleotide is a stereoblockmer.

In some embodiments, a provided oligonucleotide is a gapmer.

In some embodiments, a provided oligonucleotide is a skipmer.

In some embodiments, a provided oligonucleotide is a hemimer. In some embodiments, a hemimer is an APOC3 oligonucleotide wherein the 5′-end or the 3′-end region has a sequence that possesses a structure feature that the rest of the oligonucleotide does not have. In some embodiments, the 5′-end or the 3′-end region has or comprises 2 to 20 nucleotides. In some embodiments, a structural feature is a base modification. In some embodiments, a structural feature is a sugar modification. In some embodiments, a structural feature is a P-modification. In some embodiments, a structural feature is stereochemistry of the chiral internucleotidic linkage. In some embodiments, a structural feature is or comprises a base modification, a sugar modification, a P-modification, or stereochemistry of the chiral internucleotidic linkage, or combinations thereof. In some embodiments, a hemimer is an APOC3 oligonucleotide in which each sugar moiety of the 5′-end region shares a common modification. In some embodiments, a hemimer is an APOC3 oligonucleotide in which each sugar moiety of the 3′-end region shares a common modification. In some embodiments, a common sugar modification of the 5′ or 3′-end region is not shared by any other sugar moieties in the oligonucleotide. In some embodiments, an example hemimer is an APOC3 oligonucleotide comprising a sequence of substituted or unsubstituted 2′-O-alkyl sugar modified nucleosides, bicyclic sugar modified nucleosides, β-D-ribonucleosides or β-D-deoxyribonucleosides (for example 2′-MOE modified nucleosides, and LNA™ or ENA™ bicyclic sugar modified nucleosides) at one terminus region and a sequence of nucleosides with a different sugar moiety (such as a substituted or unsubstituted 2′-O-alkyl sugar modified nucleosides, bicyclic sugar modified nucleosides or natural ones) at the other terminus region. In some embodiments, a provided oligonucleotide is a combination of one or more of unimer, altmer, blockmer, gapmer, hemimer and skipmer. In some embodiments, a provided oligonucleotide is a combination of one or more of unimer, altmer, blockmer, gapmer, and skipmer. For instance, in some embodiments, a provided oligonucleotide is both an altmer and a gapmer. In some embodiments, a provided nucleotide is both a gapmer and a skipmer. One of skill in the chemical and synthetic arts will recognize that numerous other combinations of patterns are available and are limited only by the commercial availability and/or synthetic accessibility of constituent parts required to synthesize a provided oligonucleotide in accordance with methods of the present disclosure. In some embodiments, a hemimer structure provides advantageous benefits. In some embodiments, provided oligonucleotides are 5′-hemimers that comprises modified sugar moieties in a 5′-end sequence. In some embodiments, provided oligonucleotides are 5′-hemimers that comprises modified 2′-sugar moieties in a 5′-end sequence.

In some embodiments, a provided oligonucleotide comprises one or more optionally substituted nucleotides. In some embodiments, a provided oligonucleotide comprises one or more modified nucleotides. In some embodiments, a provided oligonucleotide comprises one or more optionally substituted nucleosides. In some embodiments, a provided oligonucleotide comprises one or more modified nucleosides. In some embodiments, a provided oligonucleotide comprises one or more optionally substituted LNAs.

In some embodiments, a provided oligonucleotide comprises one or more optionally substituted nucleobases. In some embodiments, a provided oligonucleotide comprises one or more optionally substituted natural nucleobases. In some embodiments, a provided oligonucleotide comprises one or more optionally substituted modified nucleobases. In some embodiments, a provided oligonucleotide comprises one or more 5-methylcytidine; 5-hydroxymethylcytidine, 5-formylcytosine, or 5-carboxylcytosine. In some embodiments, a provided oligonucleotide comprises one or more 5-methylcytidine.

In some embodiments, each base (BA) is independently an optionally substituted or protected nucleobase of adenine, cytosine, guanosine, thymine, or uracil. As appreciated by those skilled in the art, various protected nucleobases, including those widely known in the art, for example, those used in oligonucleotide preparation (e.g., protected nucleobases of WO/2010/064146, WO/2011/005761, WO/2013/012758, WO/2014/010250, US2013/0178612, WO/2014/012081, WO/2015/107425, WO2017/015555, and WO2017/062862, protected nucleobases of each of which are incorporated herein by reference), and can be utilized in accordance with the present disclosure.

In some embodiments, a provided oligonucleotide comprises one or more optionally substituted sugars. In some embodiments, a provided oligonucleotide comprises one or more optionally substituted sugars found in naturally occurring DNA and RNA. In some embodiments, a provided oligonucleotide comprises one or more optionally substituted ribose or deoxyribose. In some embodiments, a provided oligonucleotide comprises one or more optionally substituted ribose or deoxyribose, wherein one or more hydroxyl groups of the ribose or deoxyribose moiety is optionally and independently replaced by halogen, R′, —N(R′)₂, —OR′, or —SR′, wherein each R′ is independently as defined above and described herein. In some embodiments, a provided oligonucleotide comprises one or more optionally substituted deoxyribose, wherein the 2′ position of the deoxyribose is optionally and independently substituted with halogen, R′, —N(R′)₂, —OR′, or —SR′, wherein each R′ is independently as defined above and described herein. In some embodiments, a provided oligonucleotide comprises one or more optionally substituted deoxyribose, wherein the 2′ position of the deoxyribose is optionally and independently substituted with halogen. In some embodiments, a provided oligonucleotide comprises one or more optionally substituted deoxyribose, wherein the 2′ position of the deoxyribose is optionally and independently substituted with one or more —F. halogen. In some embodiments, a provided oligonucleotide comprises one or more optionally substituted deoxyribose, wherein the 2′ position of the deoxyribose is optionally and independently substituted with —OR′, wherein each R′ is independently as defined above and described herein. In some embodiments, a provided oligonucleotide comprises one or more optionally substituted deoxyribose, wherein the 2′ position of the deoxyribose is optionally and independently substituted with —OR′, wherein each R′ is independently an optionally substituted C₁-C₆ aliphatic. In some embodiments, a provided oligonucleotide comprises one or more optionally substituted deoxyribose, wherein the 2′ position of the deoxyribose is optionally and independently substituted with —OR′, wherein each R′ is independently an optionally substituted C₁-C₆ alkyl. In some embodiments, a provided oligonucleotide comprises one or more optionally substituted deoxyribose, wherein the 2′ position of the deoxyribose is optionally and independently substituted with —OMe. In some embodiments, a provided oligonucleotide comprises one or more optionally substituted deoxyribose, wherein the 2′ position of the deoxyribose is optionally and independently substituted with —O— methoxyethyl.

In some embodiments, a provided oligonucleotide is a hybridized oligonucleotide strand. In certain embodiments, a provided oligonucleotide is a partially hybridized oligonucleotide strand. In certain embodiments, a provided oligonucleotide is a completely hybridized oligonucleotide strand. In certain embodiments, a provided oligonucleotide is a double-stranded oligonucleotide. In certain embodiments, a provided oligonucleotide is a triple-stranded oligonucleotide (e.g., a triplex).

In some embodiments, any one of the structures comprising an APOC3 oligonucleotide depicted in WO2012/030683 can be modified in accordance with methods of the present disclosure to provide chirally controlled compositions thereof. For example, in some embodiments, chirally controlled composition comprises a stereochemical control at any one or more of chiral linkage phosphorus atoms, optionally through incorporation of one or more P-modifications described in WO2012/030683 or the present disclosure. For example, in some embodiments, a particular nucleotide unit of an APOC3 oligonucleotide of WO2012/030683 is preselected to be provided with chiral control at the linkage phosphorus of that nucleotide unit and/or to be P-modified with chiral control at the linkage phosphorus of that nucleotide unit.

In some embodiments, a provided oligonucleotide comprises a nucleic acid analog, e.g., GNA, LNA, PNA, TNA, F-HNA (F-THP or 3′-fluoro tetrahydropyran), MNA (mannitol nucleic acid, e.g., Leumann 2002 Bioorg. Med. Chem. 10: 841-854), ANA (anitol nucleic acid), and Morpholino.

In some embodiments, a provided oligonucleotide is characterized as having the ability to indirectly or directly increase or decrease activity of a protein or inhibition or promotion of the expression of a protein. In some embodiments, a provided oligonucleotide is characterized in that it is useful in the control of cell proliferation, viral replication, and/or any other cell signaling process.

In some embodiments, the 5′-end and/or the 3′-end of a provided oligonucleotide is modified. In some embodiments, the 5′-end and/or the 3′-end of a provided oligonucleotide is modified with a terminal cap moiety. Examples of such modifications, including terminal cap moieties are extensively described herein and in the art, for example but not limited to those described in US Patent Application Publication US 2009/0023675A1.

In some embodiments, oligonucleotides of an APOC3 oligonucleotide type characterized by 1) a common base sequence and length, 2) a common pattern of backbone linkages, and 3) a common pattern of backbone chiral centers, have the same chemical structure. For example, they have the same base sequence, the same pattern of nucleoside modifications, the same pattern of backbone linkages (i.e., pattern of internucleotidic linkage types, for example, phosphate, phosphorothioate, etc), the same pattern of backbone chiral centers (i.e. pattern of linkage phosphorus stereochemistry (Rp/Sp)), and the same pattern of backbone phosphorus modifications (e.g., pattern of “—XLR¹” groups in Formula I).

Single-Stranded RNAi Agents and Antisense Oligonucleotides

In some embodiments, the present disclosure provides oligonucleotides. In some embodiments, the present disclosure provides oligonucleotides which decrease the expression and/or level of a target gene or its gene product. Those of ordinary skill in the art, reading the present disclosure, will appreciate that, in some embodiments, provided oligonucleotides may act as RNAi agents. Alternatively or additionally, in some embodiments, provided oligonucleotides may act via an RNase H-dependent mechanism and/or another biochemical mechanism that does not involve RNA interference.

Among other things, the present disclosure defines certain structural attributes that may be particularly desirable and/or effective in an APOC3 oligonucleotide. Among other things, the present disclosure defines certain structural attributes that may be particularly desirable and/or effective in an APOC3 oligonucleotide that acts as an RNAi agent. In some embodiments, the present disclosure defines certain structural attributes that may be particularly desirable and/or effective in an APOC3 oligonucleotide that acts via an RNase H-dependent mechanism and/or other biochemical mechanism. In some embodiments, the present disclosure defines certain structural attributes that may be particularly desirable and/or effective in a single-stranded ssRNAi agent (ssRNAi or ssRNAi agent); in some such embodiments, as described further herein below, such structural attributes may be distinct from those that are particularly desirable and/or effective in a corresponding strand of a double-stranded RNAi agent (dsRNAi or dsRNAi agent). In some embodiments, provided oligonucleotides are single-stranded RNAi agents (e.g., which can be loaded into RISC and/or can direct or enhance RISC-mediated target). In some embodiments, provided oligonucleotides are antisense oligonucleotides (e.g., which can be loaded into RNase H and/or direct or enhance RNase-H-mediated cleavage of a target and/or operate via a different biochemical mechanism).

In some embodiments (including in some single-stranded oligonucleotide embodiments), oligonucleotides that act as RNAi agents may have one or more different structural attributes and/or functional properties from those oligonucleotides that act via an RNase H-dependent mechanism. In some embodiments, an APOC3 oligonucleotide can direct a decrease in the expression and/or level of a target gene or its gene product by sterically blocking translation after binding to a target gene mRNA, and/or by altering or interfering with mRNA splicing and/or exon inclusion or exclusion (e.g., skipping). In some embodiments, an APOC3 oligonucleotide can perform a function, or a significant percentage of a function (for example, 10-100%, no less than 10%, 20%, 30%, 40%, 50%, 60%, 70%, 80%, 90% percent or more) independent of RNA interference or RISC.

In some embodiments, a provided oligonucleotide is an antisense oligonucleotide (ASO) which directs cleavage of a target RNA mediated by RNase H and not RISC (RNA interference silencing complex).

In some embodiments, a provided oligonucleotide is a single-stranded RNAi (ssRNAi) agent which directs cleavage of a target mRNA mediated by the RISC (RNA interference silencing complex) and not the enzyme RNase H. In some embodiments, an APOC3 oligonucleotide can perform a function, or a significant percentage of a function (for example, 10-100%, no less than 10%, 20%, 30%, 40%, 50%, 60%, 70%, 80%, 90% percent or more) independent of RNase H.

A double-stranded RNAi agent can also direct cleavage of a target mRNA using RISC and not the enzyme RNase H. In some embodiments, a single-stranded RNAi agent differs from a double-stranded RNAi agent in that a ssRNAi agent includes only a single oligonucleotide strand and generally does not comprise a double-stranded region of significant length, and a dsRNAi agent comprises a double stranded region of significant length (e.g., at least about 15 bp, or about 19 bp in a “canonical” siRNA). In some embodiments, a dsRNAi comprises two separate, complementary strands (which are not covalently linked) which form a double-stranded region (e.g., in a “canonical” siRNA), or a long single strand which comprises two complementary sequences which together form a double-stranded region (e.g., in a shRNA or short hairpin RNA). In some embodiments of a dsRNAi, the passenger strand has a single-stranded nick, forming two strands. In some embodiments, the present disclosure demonstrates that sequences and/or structural elements (chemical modifications, stereochemistry, etc.) required for efficacious single-stranded RNAi agents may differ from those required for efficacious double-stranded RNAi agents.

Among other things, the present disclosure encompasses the recognition that certain designs (e.g., sequences and/or structural elements) which may be suitable for double-stranded RNAi agents may not be suitable for single-stranded RNAi agents (including single-stranded RNAi agents of provided formats described herein), and vice versa. In some embodiments, the present disclosure provides designs for effective ssRNAi. In some embodiments, the present disclosure demonstrates that certain base sequences, when combined with structural elements (modifications, stereochemistry, additional chemical moiety or moieties, etc.) in accordance with the present disclosure, can provided oligonucleotides having unexpectedly high activities, for example, when administered as ssRNAi agents, particularly in comparison with oligonucleotides comprising the same sequences but double-stranded and administered as dsRNAi agents. In some embodiments, the present disclosure demonstrates that certain base sequences, when combined with structural elements (modifications, stereochemistry, additional chemical moiety or moieties, etc.) in accordance with the present disclosure, can provided oligonucleotides having unexpectedly high activities, for example, the ability to decrease the expression and/or level of a target gene or its gene product.

Structural and Functional Differences Between Single-Stranded RNAi (ssRNAi) Agents, Double-Stranded RNAi (dsRNAi) Agents, and RNase H-Dependent Antisense Oligonucleotides (ASOs)

In some embodiments, single-stranded RNAi (ssRNAi) agents, double-stranded RNAi (dsRNAi) agents and RNase H-dependent antisense oligonucleotides (ASOs) all involve binding of an agent or oligonucleotide (or portion thereof) to a complementary (or substantially complementary) target RNA (e.g., a mRNA or pre-mRNA), followed by cleavage of the target RNA and/or a decrease the expression and/or level of a target gene or its gene product. In some embodiments, RNAi agents, whether double- or single-stranded, employ the RISC, or RNA interference silencing complex, which includes the enzyme Ago-2 (Argonaute-2). In some embodiments, RNase H-dependent antisense oligonucleotides are single-stranded and employ a different enzyme, RNase H. RNAse H is reportedly a cellular endonuclease which cleaves the RNA strand of an RNA:DNA duplex; see U.S. Pat. No. 7,919,472. See also, Saetrom (2004 Bioinformatics 20: 3055-3063); Kretschmer-Kazemi Far et al. (2003 Nucleic Acids 31: 4417-4424); Bertrand et al. (2002) Biochem. Biophys. Res. Comm. 296: 1000-1004); Vickers et al. (2003 J. Biol. Chem. 278: 7108). In some embodiments, oligonucleotides that can direct RNase H-mediated knockdown include, but are not limited to, those consisting of or comprising a region of consecutive 2′-deoxy nucleotide units which contain no 2′-modifications. In some embodiments, oligonucleotides that can direct RNase H-mediated knockdown are gap-widened oligonucleotides or gapmers. In some embodiments, a gapmer comprises an internal region comprises a plurality of nucleotides that supports RNase H cleavage and is positioned between external regions having a plurality of nucleotides that are chemically distinct from the nucleosides of the internal region. In some embodiments, a gapmer comprise a span of 2′-deoxy nucleotides containing no 2′-modifications, flanked or adjacent to one or two wings. In some embodiments, a gap directs RNase H cleavage of the corresponding RNA target. In some embodiments, the wings do not direct or act as substrates for RNase H cleavage. The wings can be of varying lengths (including, but not limited to, 1 to 8 nt) and can comprise various modifications or analogs (including, but not limited to, 2′-modifications, including, but not limited to, 2′-OMe and 2′-MOE). See, as non-limiting examples, U.S. Pat. Nos. 9,550,988; 7,919,472; 5,013,830; 5,149,797; 5,220,007; 5,256,775; 5,366,878; 5,403,711; 5,491,133; 5,565,350; 5,623,065; 5,652,355; 5,652,356; and 5,700,922. In some embodiments, presence of one or more such modifications or analogs may correlate with modified (e.g., increased, reduced, or altered) RNase H cleavage of a target.

In some embodiments, double-stranded RNAi agents, even the antisense strand thereof, differ structurally from a RNase H-dependent antisense oligonucleotide. In some embodiments, RNase H-dependent antisense oligonucleotides and siRNA oligonucleotides seem to have completely opposite characteristics, both regarding 5′-end structures and overall duplex stability.

Double-stranded RNAi agents can reportedly be naturally-produced in a cell by the Dicer enzyme, which cleaves larger RNA molecules, such as double-stranded RNA from invading viruses, into a dsRNA. The canonical structure of a dsRNA agent comprises two strands of RNA, each about 19 to 23 nt long, which are annealed to form an about 19-21 bp double-stranded region and two 3′ dinucleotide overhangs. For a double-stranded RNAi agent, the sense strand is reportedly unwound from the duplex before the antisense strand is incorporated into RISC. Aside from the natural separation of a double-stranded RNAi agent into antisense and sense strands, single-stranded RNAi agents have not been reported to be naturally produced in a human cell.

Among other things, the present disclosure provides the teaching that, in many cases, a single-stranded RNAi agent is not simply an isolated antisense strand of a double-stranded RNAi agent in that, for example, an antisense strand of an effective dsRNAi agent may be much less effective than the dsRNAi agent, and a ssRNAi agent, when formulated as a dsRNAi agent (for example, by annealing with a sense strand), may be much less effective than the ssRNAi agent. In some embodiments, double-stranded and single-stranded RNAi agents differ in many significant ways. Structural parameters of double-stranded RNAi agents are not necessarily reflected in single-stranded RNAi agents.

In some embodiments, the present disclosure teaches that target sequences which are suitable for double-stranded RNAi agents may not be suitable for single-stranded RNAi agents, and vice versa. For example, in at least some cases, single-stranded versions of double-stranded RNAi agents may not be efficacious. As a non-limiting example, Table 46A shows that several ssRNAi agents were constructed with sequences derived from dsRNAi. These ssRNAi based on dsRNAi were generally less efficacious than the corresponding dsRNAi.

In some embodiments, double-stranded and single-stranded RNAi agents also differ in their sensitivity to incorporation of chirally controlled internucleotidic linkages. For example, Matranga et al. (2005 Cell 123: 607-620) reported that introduction of a single Sp internucleotidic linkage (e.g., a single Sp PS) into the sense strand of a double-stranded RNAi agent greatly decreased RISC assembly and RNA interference activity. In contrast, in some embodiments, data shown herein demonstrate that, surprisingly, incorporation of a Sp internucleotidic linkage)(e.g., Sp PS) can perform two functions for a single-stranded RNAi agent: (a) it increases stability against nucleases; and (b) does not interfere with RNA interference activity. Many example oligonucleotides can perform as efficacious single-stranded RNAi agents comprising one or more chirally controlled internucleotidic linkages (e.g., Sp internucleotidic linkages, or Sp PS (phosphorothioate) are shown herein).

Alternatively or additionally, double-stranded and single-stranded RNAi agents can differ in immunogenicity. In some embodiments, some single-stranded RNAi agents are reportedly more immunogenic than double-stranded RNAi agents. Sioud J. Mol. Biol. (2005) 348, 1079-1090. In some embodiments, several double-stranded RNAi agents reportedly did not induce an immune response, whereas corresponding single-stranded RNAi agents did. In some embodiments, the present disclosure provides oligonucleotides with low immunogenicity. In some embodiments, such oligonucleotides can be utilized as ssRNAi reagent.

Among other things, the present disclosure encompasses the recognition that certain conventional designs of single-stranded RNAi agents, which derive single-stranded RNAi agents, including base sequences, from double-stranded RNAi agents, often fail to provide effective single-stranded RNAi agents. In some embodiments, the present disclosure demonstrates that, surprisingly, ssRNAi agents derived from base sequences of effective RNase H-dependent ASOs can produce efficacious ssRNAi agents (see Table 46A).

In some embodiments, the present disclosure provides oligonucleotides which can be utilized as efficacious RNase-H dependent ASOs, which comprise regions of 2′-deoxy nucleotides without 2′-modifications, and which are complementary or substantially complementary to RNA sequences or portions thereof. In some embodiments, a region can be, for example, a core sequence of about 10 nt flanked on one or both sides by wings, wherein the wings differ from the core in chemistry and can comprise, as non-limiting examples, 2′-modifications or internucleotidic linkage modifications.

Oligonucleotides

In some embodiments, provided oligonucleotides can direct a decrease in the expression and/or level of a target gene or its gene product. In some embodiments, provided oligonucleotides can direct a decrease in the expression and/or level of a target gene or its gene product via RNA interference. In some embodiments, provided oligonucleotides can direct a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides can direct a decrease in the expression and/or level of a target gene or its gene product via RNA interference and/or RNase H-mediated knockdown. In some embodiments, provided oligonucleotides can direct a decrease in the expression and/or level of a target gene or its gene product by sterically blocking translation after binding to a target gene mRNA, and/or by altering or interfering with mRNA splicing and/or exon inclusion or exclusion.

In some embodiments, a provided oligonucleotide has a structural element or format or portion thereof described herein.

In some embodiments, a provided oligonucleotide capable of directing a decrease in the expression and/or level of a target gene or its gene product has a structural element or format or portion thereof described herein.

In some embodiments, a provided oligonucleotide capable of directing a decrease in the expression and/or level of a target gene or its gene product has the format of any oligonucleotide disclosed herein, e.g., in Table 1A, or in the Figures or Tables, or otherwise disclosed herein.

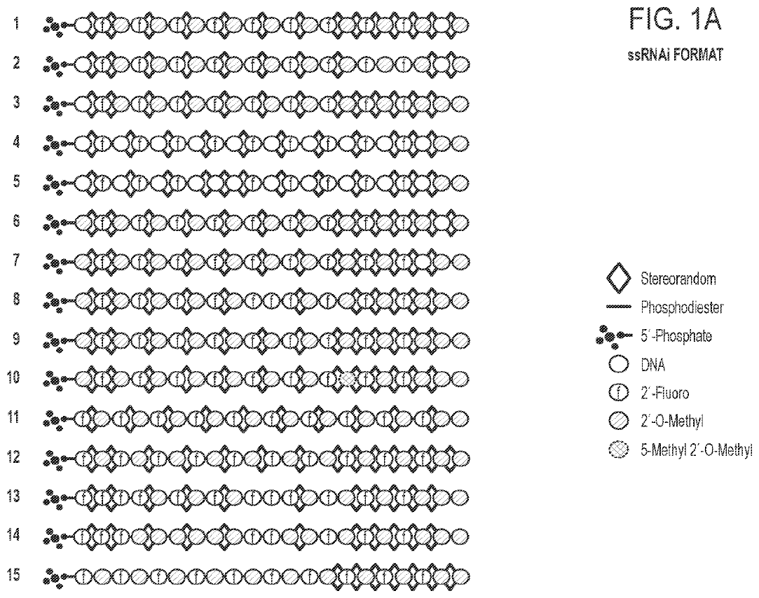

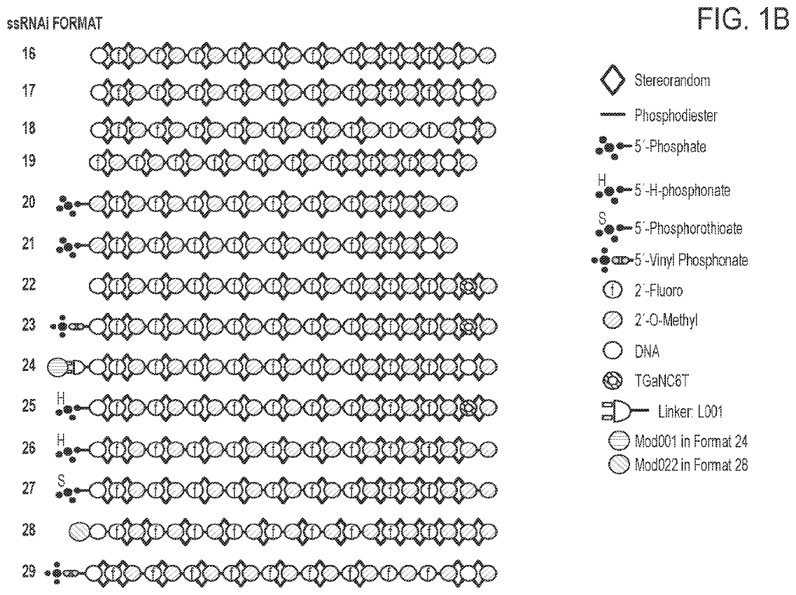

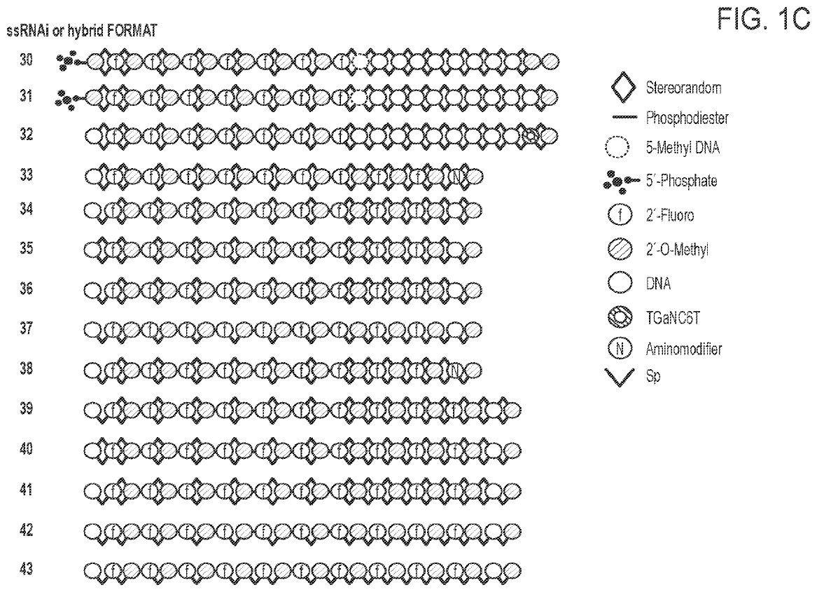

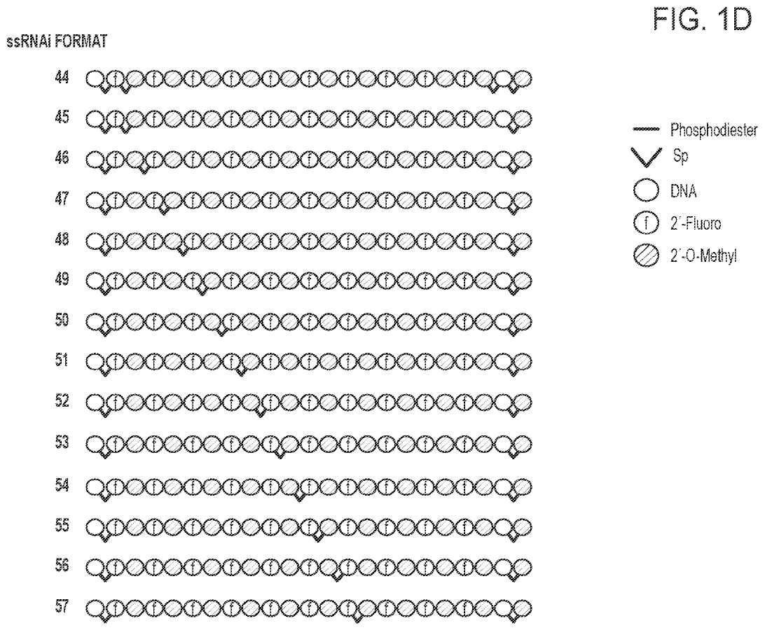

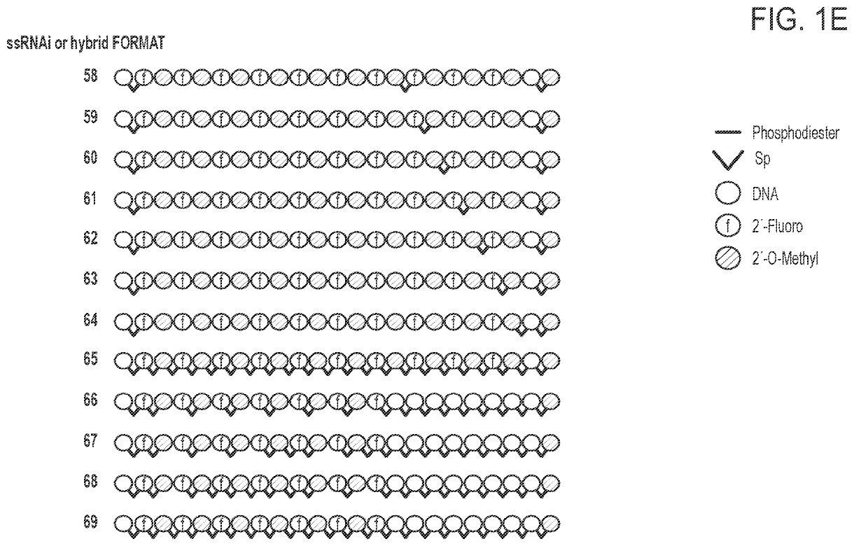

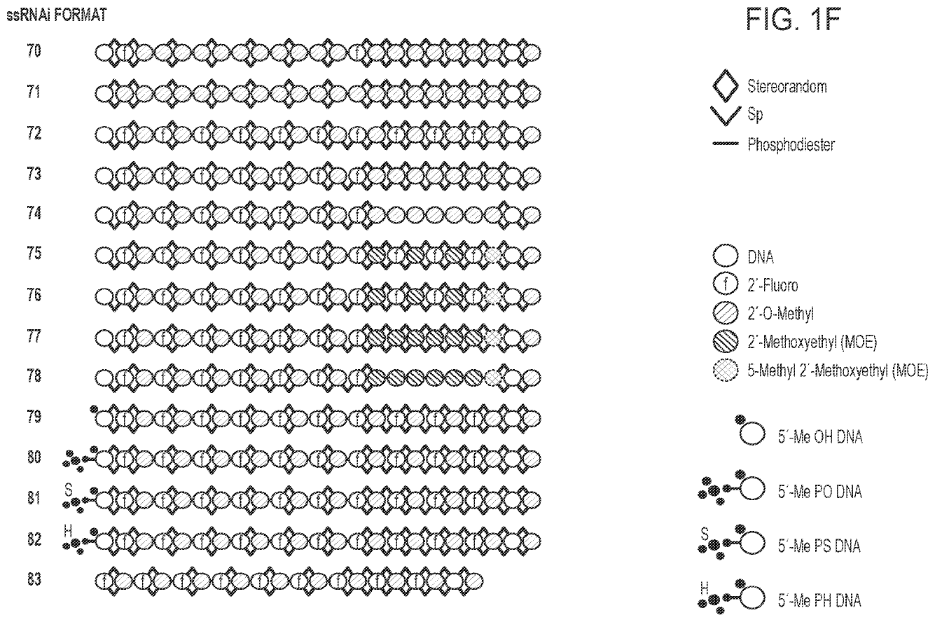

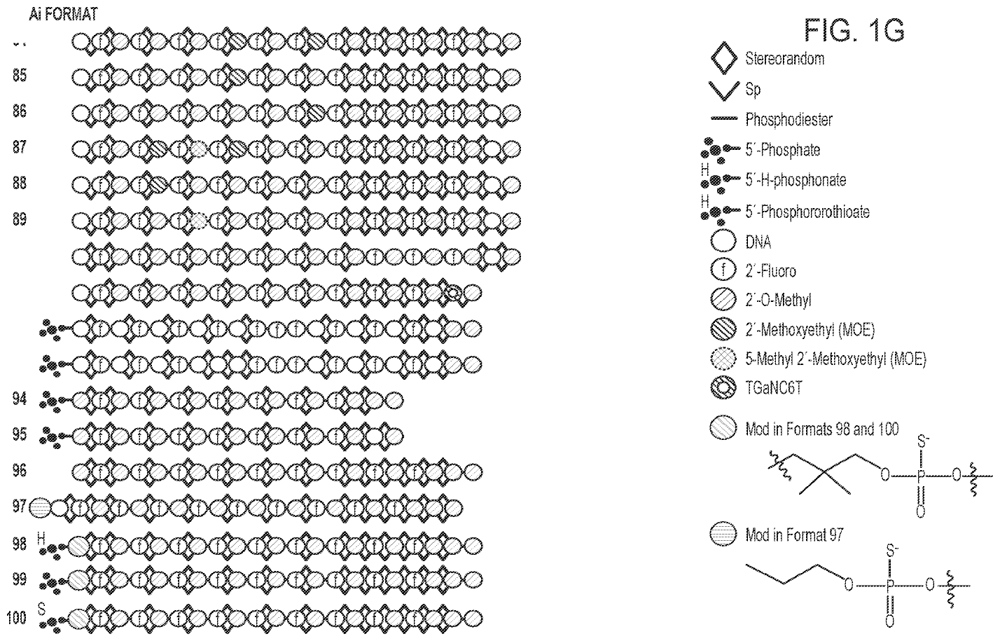

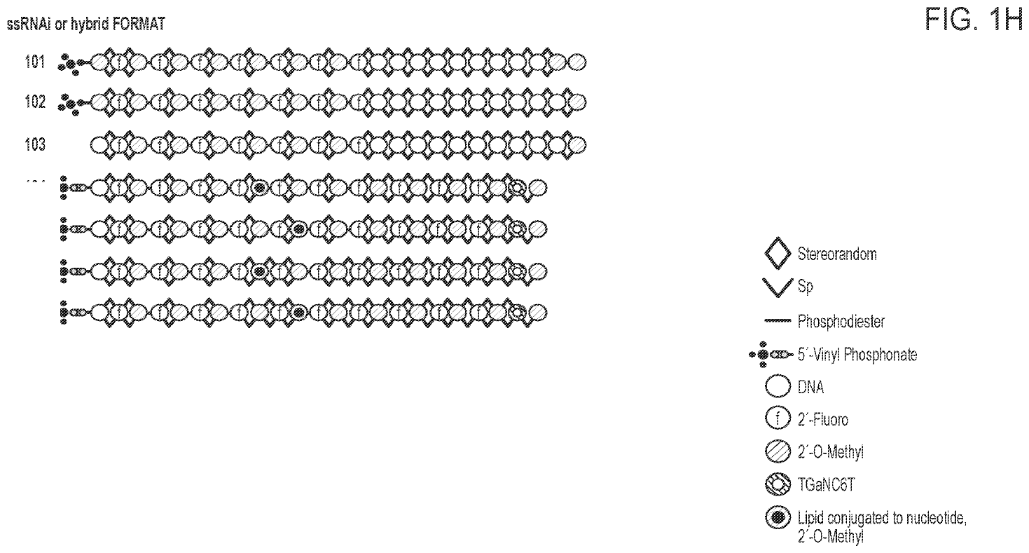

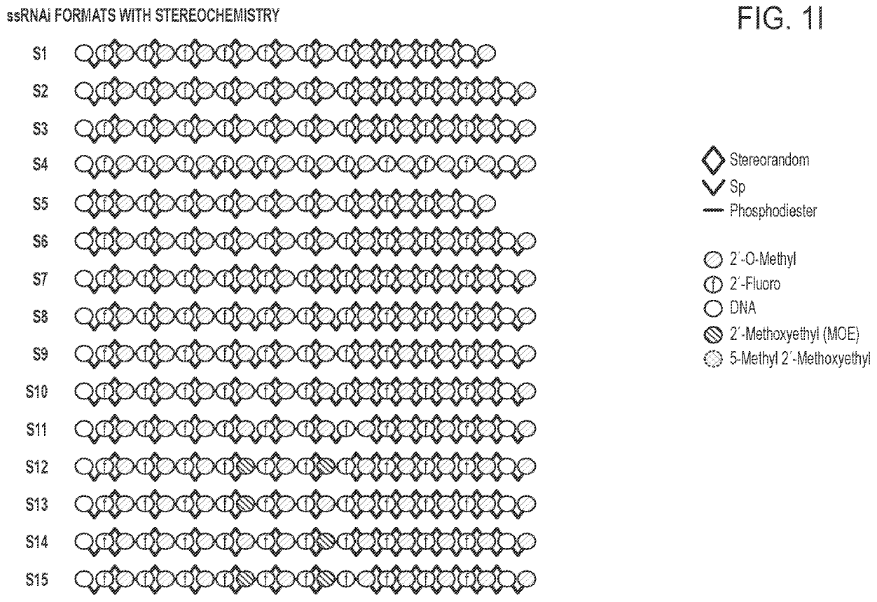

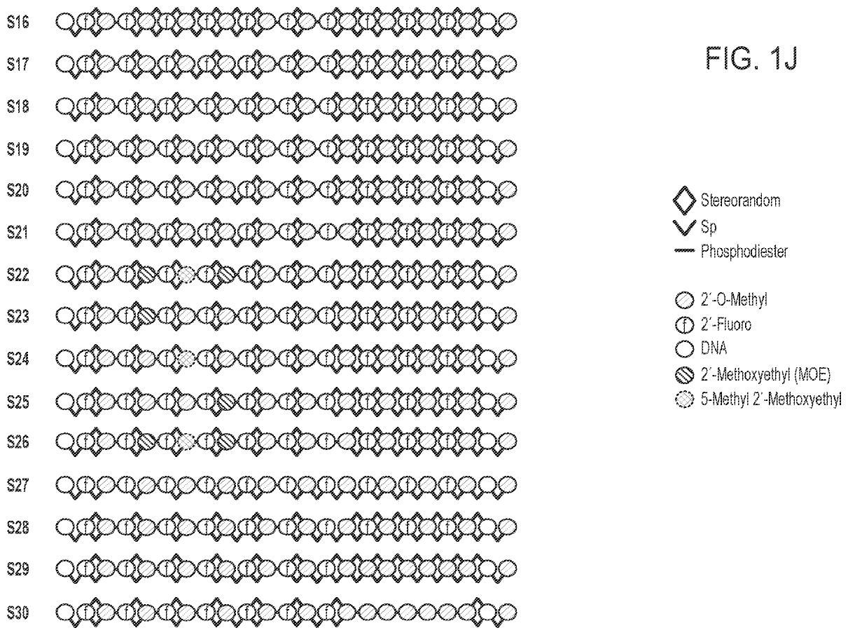

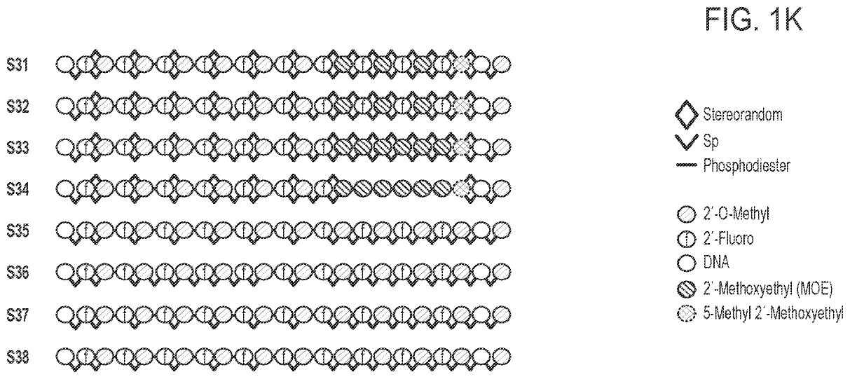

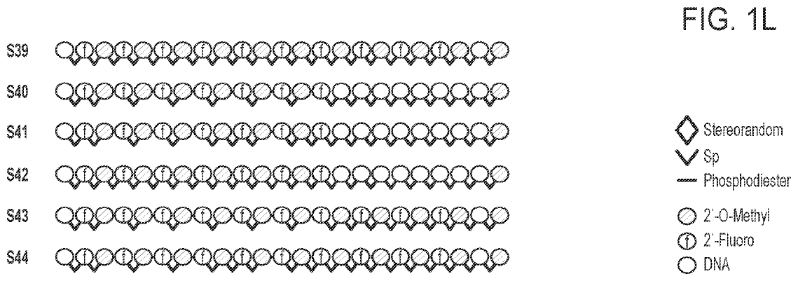

In some embodiments, a provided oligonucleotide has any of Formats illustrated in FIG. 1.

The present disclosure presents data showing that various oligonucleotides of various formats are capable of directing a decrease in the expression and/or level of a target gene or its gene product targeted against any of multiple different sequences, in multiple different genes, in multiple different species; additional data was generated supporting the efficacy of ssRNAi agents of the disclosed Formats and not shown.

In some embodiments, a provided oligonucleotide capable of directing RNase H-mediated knockdown has a structural element or format or portion thereof described herein.

In some embodiments, a provided oligonucleotide capable of directing RNase H-mediated knockdown has the format of any oligonucleotide disclosed herein, e.g., in Table 1A or in the Figures or Tables, or otherwise disclosed herein.

In some embodiments, a provided oligonucleotide has any of Formats illustrated in FIG. 1.

The present disclosure presents data showing that various oligonucleotides of various formats are capable of directing RNase H-mediated knockdown against any of multiple different sequences, in multiple different genes, in multiple different species; additional data was generated supporting the efficacy of ssRNAi agents of the disclosed Formats and not shown.

In some embodiments, a provided oligonucleotide capable of directing single-stranded RNA interference has a structural element or format or portion thereof described herein.

In some embodiments, a provided oligonucleotide capable of directing single-stranded RNA interference has the format of any oligonucleotide disclosed herein, e.g., in Table 1A or in the Figures or Tables, or otherwise disclosed herein.

In some embodiments, a provided single-stranded RNAi agent has any of the Formats illustrated in FIG. 1.

The present disclosure presents data showing that various RNAi agents of various formats are capable of directing RNA interference against any of multiple different sequences, in any of multiple different genes; additional data was generated supporting the efficacy of ssRNAi agents of the disclosed Formats and not shown.

In some embodiments, a target of RNAi is a transcript. In some embodiments, a transcript is pre-mRNA. In some embodiments, a transcript is mature RNA. In some embodiments, a transcript is mRNA. In some embodiments, a transcript comprises a mutation. In some embodiments, a mutation is a frameshift. In some embodiments, a transcript comprises a premature termination codon. In some embodiments, a target of RNAi is a RNA which is not a mRNA. In some embodiments, a target of RNAi is a non-coding RNA. In some embodiments, a target of RNAi is a long non-coding RNA. In some embodiments, provided oligonucleotides in provided compositions, e.g., oligonucleotides of a first plurality, comprise base modifications, sugar modifications, and/or internucleotidic linkage modifications. In some embodiments, provided oligonucleotides comprise base modifications and sugar modifications. In some embodiments, provided oligonucleotides comprise base modifications and internucleotidic linkage modifications. In some embodiments, provided oligonucleotides comprise sugar modifications and internucleotidic modifications. In some embodiments, provided compositions comprise base modifications, sugar modifications, and internucleotidic linkage modifications. Example chemical modifications, such as base modifications, sugar modifications, internucleotidic linkage modifications, etc. are widely known in the art including but not limited to those described in this disclosure. In some embodiments, a modified base is substituted A, T, C, G or U. In some embodiments, a sugar modification is 2′-modification. In some embodiments, a 2′-modification is 2-F modification. In some embodiments, a 2′-modification is 2′-OR¹. In some embodiments, a 2′-modification is 2′-OR¹, wherein R¹ is optionally substituted alkyl. In some embodiments, a 2′-modification is 2′-OMe. In some embodiments, a 2′-modification is 2′-MOE. In some embodiments, a modified sugar moiety is a bridged bicyclic or polycyclic ring. In some embodiments, a modified sugar moiety is a bridged bicyclic or polycyclic ring having 5-20 ring atoms wherein one or more ring atoms are optionally and independently heteroatoms. Example ring structures are widely known in the art, such as those found in BNA, LNA, etc. In some embodiments, provided oligonucleotides comprise both one or more modified internucleotidic linkages and one or more natural phosphate linkages. In some embodiments, oligonucleotides comprising both modified internucleotidic linkage and natural phosphate linkage and compositions thereof provide improved properties, e.g., activities, etc. In some embodiments, a modified internucleotidic linkage is a chiral internucleotidic linkage. In some embodiments, a modified internucleotidic linkage is a phosphorothioate linkage. In some embodiments, a modified internucleotidic linkage is a substituted phosphorothioate linkage.

Among other things, the present disclosure encompasses the recognition that stereorandom oligonucleotide preparations contain a plurality of distinct chemical entities that differ from one another, e.g., in the stereochemical structure of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence, they are different chemical entities at least due to their different backbone stereochemistry, and they can have, as demonstrated herein, different properties, e.g., activities, etc. Among other things, the present disclosure provides new compositions that are or contain particular stereoisomers of oligonucleotides of interest. In some embodiments, a particular stereoisomer may be defined, for example, by its base sequence, its length, its pattern of backbone linkages, and its pattern of backbone chiral centers. As is understood in the art, in some embodiments, base sequence may refer to the identity and/or modification status of nucleoside residues (e.g., of sugar and/or base components, relative to standard naturally occurring nucleotides such as adenine, cytosine, guanosine, thymine, and uracil) in an APOC3 oligonucleotide and/or to the hybridization character (i.e., the ability to hybridize with particular complementary residues) of such residues. In some embodiments, the present disclosure provide an APOC3 oligonucleotide composition comprising a predetermined level of oligonucleotides of an individual oligonucleotide type which are chemically identical, e.g. they have the same base sequence, the same pattern of nucleoside modifications (modifications to sugar and base moieties, if any), the same pattern of backbone chiral centers, and the same pattern of backbone phosphorus modifications. The present disclosure demonstrates, among other things, that individual stereoisomers of a particular oligonucleotide can show different stability and/or activity from each other. In some embodiments, property improvements achieved through inclusion and/or location of particular chiral structures within an APOC3 oligonucleotide can be comparable to, or even better than those achieved through use of particular backbone linkages, residue modifications, etc. (e.g., through use of certain types of modified phosphates [e.g., phosphorothioate, substituted phosphorothioate, etc.], sugar modifications [e.g., 2′-modifications, etc.], and/or base modifications [e.g., methylation, etc.]). Among other things, the present disclosure recognizes that, in some embodiments, properties (e.g., activities, etc.) of an APOC3 oligonucleotide can be adjusted by optimizing its pattern of backbone chiral centers, optionally in combination with adjustment/optimization of one or more other features (e.g., linkage pattern, nucleoside modification pattern, etc.) of the oligonucleotide. As exemplified by various examples in the present disclosure, provided chirally controlled oligonucleotide compositions can demonstrate improved properties, e.g., improved single-stranded RNA interference activity, RNase H-mediated knockdown, improved delivery, etc.

In some embodiments, oligonucleotide properties can be adjusted by optimizing stereochemistry (pattern of backbone chiral centers) and chemical modifications (modifications of base, sugar, and/or internucleotidic linkage) or patterns thereof.

In some embodiments, a common pattern of backbone chiral centers (e.g., a pattern of backbone chiral centers in a single-stranded RNAi agent) comprises a pattern of OSOSO, OSSSO, OSSSOS, SOSO, SOSO, SOSOS, SOSOSO, SOSOSOSO, SOSSSO, SSOSSSOSS, SSSOSOSSS, SSSSOSOSSSS, SSSSS, SSSSSS, SSSSSSS, SSSSSSSS, SSSSSSSSS, or RRR, wherein S represents a phosphorothioate in the Sp configuration, and O represents a phosphodiester. wherein R represents a phosphorothioate in the Rp configuration.

In some embodiments, the non-chiral center is a phosphodiester linkage. In some embodiments, the chiral center in a Sp configuration is a phosphorothioate linkage. In some embodiments, the non-chiral center is a phosphodiester linkage. In some embodiments, the chiral center in a Sp configuration is a phosphorothioate linkage.

In some embodiments, a provided oligonucleotide comprises any pattern of stereochemistry described herein. In some embodiments, a provided oligonucleotide comprises any pattern of stereochemistry described herein and is capable of directing RNA interference. In some embodiments, a provided oligonucleotide comprises any pattern of stereochemistry described herein and is capable of directing RNase H-mediated knockdown. In some embodiments, a provided oligonucleotide comprises any pattern of stereochemistry described herein and is capable of directing RNA interference and RNase H-mediated knockdown. In some embodiments, a provided oligonucleotide comprises any pattern of stereochemistry described herein and is capable of directing RNA interference, wherein the pattern of stereochemistry is in the seed and/or post-seed region. In some embodiments, a provided oligonucleotide comprises any pattern of stereochemistry described herein and is capable of directing RNA interference and RNase H-mediated knockdown, wherein the pattern of stereochemistry is in the seed and/or post-seed region.

In some embodiments, a provided oligonucleotide comprises any modification or pattern of modification described herein. In some embodiments, a provided oligonucleotide comprises any modification or pattern of modification described herein and is capable of directing RNA interference. In some embodiments, a provided oligonucleotide comprises any pattern of modification described herein and is capable of directing RNase H-mediated knockdown. In some embodiments, a provided oligonucleotide comprises any pattern of modification described herein and is capable of directing RNA interference and RNase H-mediated knockdown. In some embodiments, a provided oligonucleotide comprises any pattern of modification described herein and is capable of directing RNA interference, wherein the pattern of modification is in the seed and/or post-seed region. In some embodiments, a provided oligonucleotide comprises any pattern of modification described herein and is capable of directing RNA interference and RNase H-mediated knockdown, wherein the pattern of modification is in the seed and/or post-seed region. In some embodiments, a modification or pattern of modification is a modification or pattern of modifications at the 2′ position of a sugar. In some embodiments, a modification or pattern of modification is a modification or pattern of modifications of sugars, e.g., at the 2′ position of a sugar, including but not limited to, 2′-deoxy, 2′-F, 2′-OMe, 2′-MOE, and 2′-OR1, wherein R1 is optionally substituted C1-6 alkyl.

In some embodiments, the present disclosure demonstrates that 2′-F modifications, among other things, can improve single-stranded RNA interference. In some embodiments, the present disclosure demonstrates that Sp internucleotidic linkages, among other things, at the 5′- and 3′-ends can improve oligonucleotide stability. In some embodiments, the present disclosure demonstrates that, among other things, natural phosphate linkages and/or Rp internucleotidic linkages can improve removal of oligonucleotides from a system. As appreciated by a person having ordinary skill in the art, various assays known in the art can be utilized to assess such properties in accordance with the present disclosure.

In some embodiments, provided oligonucleotides capable of directing single-stranded RNA interference comprise one or more modified sugar moieties. In some embodiments, 5% or more of the sugar moieties of provided oligonucleotides are modified.

In some embodiments, the present disclosure provides an APOC3 oligonucleotide composition comprising a first plurality of oligonucleotides, wherein:

oligonucleotides of the first plurality have the same base sequence; and

oligonucleotides of the first plurality comprise one or more modified sugar moieties, or comprise one or more natural phosphate linkages and one or more modified internucleotidic linkages.

In some embodiments, oligonucleotides of the first plurality comprise one or more modified sugar moieties. In some embodiments, provided oligonucleotides comprise one or more modified sugar moieties.

In some embodiments, provided compositions alter transcript single-stranded RNA interference so that an undesired target and/or biological function are suppressed. In some embodiments, in such cases provided composition can also induce cleavage of the transcript after hybridization.

In some embodiments, each oligonucleotide of the first plurality comprises one or more modified sugar moieties and/or one or more modified internucleotidic linkages. In some embodiments, each oligonucleotide of the first plurality comprises no more than about 95% unmodified sugar moieties. In some embodiments, each oligonucleotide of the first plurality comprises no more than about 50% unmodified sugar moieties. In some embodiments, each oligonucleotide of the first plurality comprises no more than about 5% unmodified sugar moieties. In some embodiments, each sugar moiety of the oligonucleotides of the first plurality is independently modified.

In some embodiments, each oligonucleotide of the first plurality comprises two or more modified internucleotidic linkages. In some embodiments, each oligonucleotide of the first plurality comprises three or more modified internucleotidic linkages. In some embodiments, each oligonucleotide of the first plurality comprises four or more modified internucleotidic linkages. In some embodiments, each oligonucleotide of the first plurality comprises five or more modified internucleotidic linkages. In some embodiments, each oligonucleotide of the first plurality comprises ten or more modified internucleotidic linkages.

In some embodiments, each oligonucleotide of the first plurality comprises no more than about 30% natural phosphate linkages. In some embodiments, each oligonucleotide of the first plurality comprises no more than about 20% natural phosphate linkages. In some embodiments, each oligonucleotide of the first plurality comprises no more than about 10% natural phosphate linkages. In some embodiments, each oligonucleotide of the first plurality comprises no more than about 5% natural phosphate linkages.

In some embodiments, provided oligonucleotides contain increased levels of one or more isotopes. In some embodiments, provided oligonucleotides are labeled, e.g., by one or more isotopes of one or more elements, e.g., hydrogen, carbon, nitrogen, etc. In some embodiments, provided oligonucleotides in provided compositions, e.g., oligonucleotides of a first plurality, comprise base modifications, sugar modifications, and/or internucleotidic linkage modifications, wherein the oligonucleotides contain an enriched level of deuterium. In some embodiments, provided oligonucleotides are labeled with deuterium (replacing —¹H with —²H) at one or more positions. In some embodiments, one or more ¹H of an APOC3 oligonucleotide or any moiety conjugated to the oligonucleotide (e.g., a targeting moiety, lipid moiety, etc.) is substituted with ²H. Such oligonucleotides can be used in any composition or method described herein.

The present invention includes all pharmaceutically acceptable isotopically-labelled compounds wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I, ¹²⁴I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Tomography (PET) studies for examining substrate receptor occupancy.

Isotopically-labelled compounds can generally be prepared by conventional techniques known to those skilled in the art or by processes analogous to those described in the accompanying Examples and Preparations using an appropriate isotopically-labelled reagents in place of the non-labelled reagent previously employed.

The compounds of the present invention may contain asymmetric or chiral centers, and, therefore, exist in different stereoisomeric forms. Unless specified otherwise, it is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans-forms, as well as mixtures, are embraced within the scope of the invention.

Chiral compounds of the invention (and chiral precursors thereof) may be obtained in enantiomerically-enriched form using chromatography, typically high pressure liquid chromatography (HPLC) or supercritical fluid chromatography (SFC), on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine (DEA) or isopropylamine. Concentration of the eluent affords the enriched mixture.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g. chiral auxiliary such as a chiral alcohol or Mosher's acid chloride), separating the diastereoisomers and converting (e.g. hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

In some embodiments, controlling structural elements of oligonucleotides, such as chemical modifications (e.g., modifications of a sugar, base and/or internucleotidic linkage) or patterns thereof, alterations in stereochemistry (e.g., stereochemistry of a backbone chiral internucleotidic linkage) or patterns thereof, substitution of an atom with an isotope of the same element, and/or conjugation with an additional chemical moiety (e.g., a lipid moiety, targeting moiety, etc.) can have a significant impact on a desired biological effect. In some embodiments, a desired biological effect is enhanced by more than 2 fold.

In some embodiments, a desired biological effect is directing a decrease in the expression and/or level of a target gene or its gene product. In some embodiments, a desired biological effect is improved single-stranded RNA interference. In some embodiments, a desired biological effect is improved RNase H-mediated knockdown. In some embodiments, a desired biological effect is improved single-stranded RNA interference and/or RNase H-mediated knockdown.

In some embodiments, the present disclosure provides an APOC3 oligonucleotide composition comprising a first plurality of oligonucleotides which:

1) have a common base sequence complementary to a target sequence in a transcript; and

2) comprise one or more modified sugar moieties and modified internucleotidic linkages.

In some embodiments, a provided oligonucleotide composition is characterized in that, when it is contacted with the transcript in a single-stranded RNA interference system, RNAi-mediated knockdown of the transcript is improved relative to that observed under reference conditions selected from the group consisting of absence of the composition, presence of a reference composition, and combinations thereof.

In some embodiments, the present disclosure provides an APOC3 oligonucleotide composition comprising a first plurality of oligonucleotides capable of directing single-stranded RNA interference, wherein an APOC3 oligonucleotides type is defined by:

1) base sequence;

2) pattern of backbone linkages;

3) pattern of backbone chiral centers; and

4) pattern of backbone phosphorus modifications,

which composition is chirally controlled in that it is enriched, relative to a substantially racemic preparation of oligonucleotides having the same base sequence, for oligonucleotides of the particular oligonucleotide type,

the oligonucleotide composition being characterized in that, when it is contacted with the transcript in a single-stranded RNA interference system, RNAi-mediated knockdown of the transcript is improved relative to that observed under reference conditions selected from the group consisting of absence of the composition, presence of a reference composition, and combinations thereof.

In some embodiments, each of the consecutive nucleoside units is independently preceded and/or followed by a modified internucleotidic linkage. In some embodiments, each of the consecutive nucleoside units is independently preceded and/or followed by a phosphorothioate linkage. In some embodiments, each of the consecutive nucleoside units is independently preceded and/or followed by a chirally controlled modified internucleotidic linkage. In some embodiments, each of the consecutive nucleoside units is independently preceded and/or followed by a chirally controlled phosphorothioate linkage. In some embodiments, a modified internucleotidic linkage has a structure of Formula I. In some embodiments, a modified internucleotidic linkage has a structure of Formula I-a.

In some embodiments, the present disclosure provides a single-stranded RNAi agent comprising a predetermined level of a first plurality of oligonucleotides, wherein:

oligonucleotides of the first plurality have the same base sequence;

oligonucleotides of the first plurality comprise a seed region comprising 2, 3, 4, 5, 6, 7 or more consecutive Sp modified internucleotidic linkages, a post-seed region comprising 2, 3, 4, 5, 6, 7, 8, 9, 10 or more consecutive Sp modified internucleotidic linkages.

In some embodiments, a seed region comprises 2 or more consecutive Sp modified internucleotidic linkages.

In some embodiments, a modified internucleotidic linkage has a structure of Formula I. In some embodiments, a modified internucleotidic linkage has a structure of Formula I-a.

As demonstrated in the present disclosure, in some embodiments, a provided oligonucleotide composition is characterized in that, when it is contacted with the transcript in a single-stranded RNA interference system, RNAi-mediated knockdown of the transcript is improved relative to that observed under reference conditions selected from the group consisting of absence of the composition, presence of a reference composition, and combinations thereof.

In some embodiments, the present disclosure provides an APOC3 oligonucleotide composition comprising a first plurality of oligonucleotides defined by having:

1) a common base sequence and length;

2) a common pattern of backbone linkages; and

3) a common pattern of backbone chiral centers, which composition is a substantially pure preparation of a single oligonucleotide in that a predetermined level of the oligonucleotides in the composition have the common base sequence and length, the common pattern of backbone linkages, and the common pattern of backbone chiral centers.

In some embodiments, a common base sequence and length may be referred to as a common base sequence. In some embodiments, oligonucleotides having a common base sequence may have the same pattern of nucleoside modifications, e.g. sugar modifications, base modifications, etc. In some embodiments, a pattern of nucleoside modifications may be represented by a combination of locations and modifications. In some embodiments, a pattern of backbone linkages comprises locations and types (e.g., phosphate, phosphorothioate, substituted phosphorothioate, etc.) of each internucleotidic linkages. A pattern of backbone chiral centers of an APOC3 oligonucleotide can be designated by a combination of linkage phosphorus stereochemistry (Rp/Sp) from 5′ to 3′. As exemplified above, locations of non-chiral linkages may be obtained, for example, from pattern of backbone linkages.

As understood by a person having ordinary skill in the art, a stereorandom or racemic preparation of oligonucleotides is prepared by non-stereoselective and/or low-stereoselective coupling of nucleotide monomers, typically without using any chiral auxiliaries, chiral modification reagents, and/or chiral catalysts. In some embodiments, in a substantially racemic (or chirally uncontrolled) preparation of oligonucleotides, all or most coupling steps are not chirally controlled in that the coupling steps are not specifically conducted to provide enhanced stereoselectivity. An example substantially racemic preparation of oligonucleotides is the preparation of phosphorothioate oligonucleotides through sulfurizing phosphite triesters from commonly used phosphoramidite oligonucleotide synthesis with either tetraethylthiuram disulfide or (TETD) or 3H-1, 2-bensodithiol-3-one 1, 1-dioxide (BDTD), a well-known process in the art. In some embodiments, substantially racemic preparation of oligonucleotides provides substantially racemic oligonucleotide compositions (or chirally uncontrolled oligonucleotide compositions).

As understood by a person having ordinary skill in the art, in some embodiments, diastereoselectivity of a coupling or a linkage can be assessed through the diastereoselectivity of a dimer formation under the same or comparable conditions, wherein the dimer has the same 5′- and 3′-nucleosides and internucleotidic linkage.

In some embodiments, the present disclosure provides chirally controlled oligonucleotide composition of a first plurality of oligonucleotides in that the composition is enriched, relative to a substantially racemic preparation of the same oligonucleotides, for oligonucleotides of a single oligonucleotide type. In some embodiments, the present disclosure provides chirally controlled oligonucleotide composition of a first plurality of oligonucleotides in that the composition is enriched, relative to a substantially racemic preparation of the same oligonucleotides, for oligonucleotides of a single oligonucleotide type that share:

1) a common base sequence and length;

2) a common pattern of backbone linkages; and

3) a common pattern of backbone chiral centers.

In some embodiments, the present disclosure provides an APOC3 oligonucleotide composition comprising a first plurality of oligonucleotides capable of directing single-stranded RNA interference, wherein oligonucleotides are of a particular oligonucleotide type characterized by:

1) a common base sequence and length;

2) a common pattern of backbone linkages; and

3) a common pattern of backbone chiral centers;

which composition is chirally controlled in that it is enriched, relative to a substantially racemic preparation of oligonucleotides having the same base sequence and length, for oligonucleotides of the particular oligonucleotide type.

In some embodiments, oligonucleotides having a common base sequence and length, a common pattern of backbone linkages, and a common pattern of backbone chiral centers have a common pattern of backbone phosphorus modifications and a common pattern of base modifications. In some embodiments, oligonucleotides having a common base sequence and length, a common pattern of backbone linkages, and a common pattern of backbone chiral centers have a common pattern of backbone phosphorus modifications and a common pattern of nucleoside modifications. In some embodiments, oligonucleotides having a common base sequence and length, a common pattern of backbone linkages, and a common pattern of backbone chiral centers have identical structures.

In some embodiments, oligonucleotides of an APOC3 oligonucleotide type have a common pattern of backbone phosphorus modifications and a common pattern of sugar modifications. In some embodiments, oligonucleotides of an APOC3 oligonucleotide type have a common pattern of backbone phosphorus modifications and a common pattern of base modifications. In some embodiments, oligonucleotides of an APOC3 oligonucleotide type have a common pattern of backbone phosphorus modifications and a common pattern of nucleoside modifications. In some embodiments, oligonucleotides of an APOC3 oligonucleotide type are identical.

In some embodiments, oligonucleotides having a common base sequence and length, a common pattern of backbone linkages, and a common pattern of backbone chiral centers have a common pattern of backbone phosphorus modifications. In some embodiments, oligonucleotides having a common base sequence and length, a common pattern of backbone linkages, and a common pattern of backbone chiral centers have a common pattern of backbone phosphorus modifications and a common pattern of nucleoside modifications. In some embodiments, oligonucleotides having a common base sequence and length, a common pattern of backbone linkages, and a common pattern of backbone chiral centers have a common pattern of backbone phosphorus modifications and a common pattern of sugar modifications. In some embodiments, oligonucleotides having a common base sequence and length, a common pattern of backbone linkages, and a common pattern of backbone chiral centers have a common pattern of backbone phosphorus modifications and a common pattern of base modifications. In some embodiments, oligonucleotides having a common base sequence and length, a common pattern of backbone linkages, and a common pattern of backbone chiral centers have a common pattern of backbone phosphorus modifications and a common pattern of nucleoside modifications. In some embodiments, oligonucleotides having a common base sequence and length, a common pattern of backbone linkages, and a common pattern of backbone chiral centers are identical.

In some embodiments, oligonucleotides in provided compositions have a common pattern of backbone phosphorus modifications. In some embodiments, a common base sequence is a base sequence of an APOC3 oligonucleotide type. In some embodiments, a provided composition is an APOC3 oligonucleotide composition that is chirally controlled in that the composition contains a predetermined level of a first plurality of oligonucleotides of an individual oligonucleotide type, wherein an APOC3 oligonucleotide type is defined by:

1) base sequence;

2) pattern of backbone linkages;

3) pattern of backbone chiral centers; and

4) pattern of backbone phosphorus modifications.

As noted above and understood in the art, in some embodiments, the base sequence of an APOC3 oligonucleotide may refer to the identity and/or modification status of nucleoside residues (e.g., of sugar and/or base components, relative to standard naturally occurring nucleotides such as adenine, cytosine, guanosine, thymine, and uracil) in the oligonucleotide and/or to the hybridization character (i.e., the ability to hybridize with particular complementary residues) of such residues.

In some embodiments, oligonucleotides of a particular type are identical in that they have the same base sequence (including length), the same pattern of chemical modifications to sugar and base moieties, the same pattern of backbone linkages (e.g., pattern of natural phosphate linkages, phosphorothioate linkages, phosphorothioate triester linkages, and combinations thereof), the same pattern of backbone chiral centers (e.g., pattern of stereochemistry (Rp/Sp) of chiral internucleotidic linkages), and the same pattern of backbone phosphorus modifications (e.g., pattern of modifications on the internucleotidic phosphorus atom, such as —S⁻, and -L-R¹ of Formula I).

Among other things, the present disclosure recognizes that combinations of oligonucleotide structural elements (e.g., patterns of chemical modifications, backbone linkages, backbone chiral centers, and/or backbone phosphorus modifications) can provide surprisingly improved properties such as bioactivities.

In some embodiments, provided chirally controlled (and/or stereochemically pure) preparations are RNAi agent oligonucleotides.

In some embodiments, provided chirally controlled (and/or stereochemically pure) preparations are of oligonucleotides that include one or more modified backbone linkages, bases, and/or sugars.

In some embodiments, provided compositions comprise oligonucleotides containing one or more residues which are modified at the sugar moiety. In some embodiments, provided compositions comprise oligonucleotides containing one or more residues which are modified at the 2′ position of the sugar moiety (referred to herein as a “2′-modification”). Examples of such modifications are described above and herein and include, but are not limited to, 2′-OMe, 2′-MOE, 2′-LNA, 2′-F, FRNA, FANA, S-cEt, etc. In some embodiments, provided compositions comprise oligonucleotides containing one or more residues which are 2′-modified. For example, in some embodiments, provided oligonucleotides contain one or more residues which are 2′-O-methoxyethyl (2′-MOE)-modified residues. In some embodiments, provided compositions comprise oligonucleotides which do not contain any 2′-modifications. In some embodiments, provided compositions are oligonucleotides which do not contain any 2′-MOE residues. That is, in some embodiments, provided oligonucleotides are not MOE-modified. Additional example sugar modifications are described in the present disclosure.

In some embodiments, one or more is one. In some embodiments, one or more is two. In some embodiments, one or more is three. In some embodiments, one or more is four. In some embodiments, one or more is five. In some embodiments, one or more is six. In some embodiments, one or more is seven. In some embodiments, one or more is eight. In some embodiments, one or more is nine. In some embodiments, one or more is ten. In some embodiments, one or more is at least one. In some embodiments, one or more is at least two. In some embodiments, one or more is at least three. In some embodiments, one or more is at least four. In some embodiments, one or more is at least five. In some embodiments, one or more is at least six. In some embodiments, one or more is at least seven. In some embodiments, one or more is at least eight. In some embodiments, one or more is at least nine. In some embodiments, one or more is at least ten.

In some embodiments, a sugar moiety without a 2′-modification is a sugar moiety found in a natural DNA nucleoside.

A person of ordinary skill in the art understands that various regions of a target transcript can be targeted by provided compositions and methods. In some embodiments, a base sequence of provided oligonucleotides comprises an intron sequence. In some embodiments, a base sequence of provided oligonucleotides comprises an exon sequence. In some embodiments, a base sequence of provided oligonucleotides comprises an intron and an exon sequence.

As understood by a person having ordinary skill in the art, provided oligonucleotides and compositions, among other things, can target a great number of nucleic acid polymers. For instance, in some embodiments, provided oligonucleotides and compositions may target a transcript of a nucleic acid sequence, wherein a common base sequence of oligonucleotides (e.g., a base sequence of an APOC3 oligonucleotide type) comprises or is a sequence complementary to a sequence of the transcript. In some embodiments, a common base sequence comprises a sequence complimentary to a sequence of a target. In some embodiments, a common base sequence is a sequence complimentary to a sequence of a target. In some embodiments, a common base sequence comprises or is a sequence 100% complimentary to a sequence of a target. In some embodiments, a common base sequence comprises a sequence 100% complimentary to a sequence of a target. In some embodiments, a common base sequence is a sequence 100% complimentary to a sequence of a target.

In some embodiments, a common base sequence comprises or is a sequence complementary to a characteristic sequence element. In some embodiments, a common base sequence comprises a sequence complementary to a characteristic sequence element. In some embodiments, a common base sequence is a sequence complementary to a characteristic sequence element. In some embodiments, a common base sequence comprises or is a sequence 100% complementary to a characteristic sequence element. In some embodiments, a common base sequence comprises a sequence 100% complementary to a characteristic sequence element. In some embodiments, a common base sequence is a sequence 100% complementary to a characteristic sequence element. In some embodiments herein, a characteristic sequence element is, as non-limiting examples, a seed region, a post-seed region or a portion of a seed region, or a portion of a post-seed region or a 3′-terminal dinucleotide.

In some embodiments, a characteristic sequence element comprises or is a mutation. In some embodiments, a characteristic sequence element comprises a mutation. In some embodiments, a characteristic sequence element is a mutation. In some embodiments, a characteristic sequence element comprises or is a point mutation. In some embodiments, a characteristic sequence element comprises a point mutation. In some embodiments, a characteristic sequence element is a point mutation. In some embodiments, a characteristic sequence element comprises or is an SNP. In some embodiments, a characteristic sequence element comprises an SNP. In some embodiments, a characteristic sequence element is an SNP.

In some embodiments, a common base sequence 100% matches a target sequence, which it does not 100% match a similar sequence of the target sequence.

Among other things, the present disclosure recognizes that a base sequence may have impact on oligonucleotide properties. In some embodiments, a base sequence may have impact on cleavage pattern of a target when oligonucleotides having the base sequence are utilized for suppressing a target, e.g., through a pathway involving RNase H: for example, structurally similar (all phosphorothioate linkages, all stereorandom) oligonucleotides have different sequences may have different cleavage patterns.

In some embodiments, a common base sequence is a base sequence that comprises a SNP.

As a person having ordinary skill in the art understands, provided oligonucleotide compositions and methods have various uses as known by a person having ordinary skill in the art. Methods for assessing provided compositions, and properties and uses thereof, are also widely known and practiced by a person having ordinary skill in the art. Example properties, uses, and/or methods include but are not limited to those described in WO/2014/012081 and WO/2015/107425.

In some embodiments, a chiral internucleotidic linkage has the structure of Formula I. In some embodiments, a chiral internucleotidic linkage is phosphorothioate. In some embodiments, each chiral internucleotidic linkage in a single oligonucleotide of a provided composition independently has the structure of Formula I. In some embodiments, each chiral internucleotidic linkage in a single oligonucleotide of a provided composition is a phosphorothioate.

In some embodiments, oligonucleotides of the present disclosure comprise one or more modified sugar moieties. In some embodiments, oligonucleotides of the present disclosure comprise one or more modified base moieties. As known by a person of ordinary skill in the art and described in the disclosure, various modifications can be introduced to a sugar and/or moiety. For example, in some embodiments, a modification is a modification described in U.S. Pat. No. 9,006,198, WO2014/012081 and WO/2015/107425, the sugar and base modifications of each of which are incorporated herein by reference.

In some embodiments, a sugar modification is a 2′-modification. Commonly used 2′-modifications include but are not limited to 2′-OR¹, wherein R′ is not hydrogen. In some embodiments, a modification is 2′-OR, wherein R is optionally substituted aliphatic. In some embodiments, a modification is 2′-OMe. In some embodiments, a modification is 2′-O-MOE. In some embodiments, the present disclosure demonstrates that inclusion and/or location of particular chirally pure internucleotidic linkages can provide stability improvements comparable to or better than those achieved through use of modified backbone linkages, bases, and/or sugars. In some embodiments, a provided single oligonucleotide of a provided composition has no modifications on the sugars. In some embodiments, a provided single oligonucleotide of a provided composition has no modifications on 2′-positions of the sugars (i.e., the two groups at the 2′-position are either —H/—H or —H/—OH). In some embodiments, a provided single oligonucleotide of a provided composition does not have any 2′-MOE modifications.

In some embodiments, a 2′-modification is —O-L- or -L- which connects the 2′-carbon of a sugar moiety to another carbon of a sugar moiety. In some embodiments, a 2′-modification is —O-L- or -L- which connects the 2′-carbon of a sugar moiety to the 4′-carbon of a sugar moiety. In some embodiments, a 2′-modification is S-cEt. In some embodiments, a modified sugar moiety is an LNA moiety.

In some embodiments, a 2′-modification is —F. In some embodiments, a 2′-modification is FANA. In some embodiments, a 2′-modification is FRNA.

In some embodiments, a sugar modification is a 5′-modification, e.g., R-5′-Me, S-5′-Me, etc.

In some embodiments, a sugar modification changes the size of the sugar ring. In some embodiments, a sugar modification is the sugar moiety in FHNA.

In some embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In some embodiments, an ssRNAi agent is or comprises an APOC3 oligonucleotide selected from the group consisting of any ssRNAi of any format described in FIG. 1 or otherwise herein. Those skilled in the art, reading the present specification, will appreciate that the present disclosure specifically does not exclude the possibility that any oligonucleotide described herein which is labeled as a ssRNAi agent may also or alternatively operate through another mechanism (e.g., as an antisense oligonucleotide; mediating knock-down via a RNaseH mechanism; sterically hindering translation; or any other biochemical mechanism).

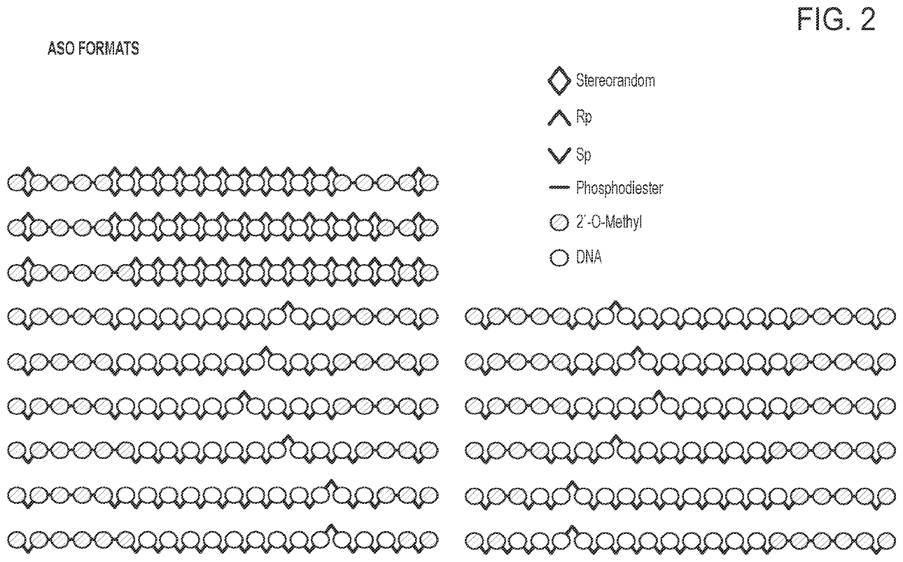

In some embodiments, an antisense oligonucleotide (ASO) is or comprises an APOC3 oligonucleotide selected from the group consisting of any oligonucleotide of any format described in FIG. 2. Those skilled in the art, reading the present specification, will appreciate that the present disclosure specifically does not exclude the possibility that any oligonucleotide described herein which is labeled as an antisense oligonucleotide (ASO) may also or alternatively operate through another mechanism (e.g., as a ssRNAi utilizing RISC); the disclosure also notes that various ASOs may operate via different mechanisms (utilizing RNaseH, sterically blocking translation or other post-transcriptional processes, changing the conformation of a target nucleic acid, etc.).

In some embodiments, a hybrid oligonucleotide is or comprises an APOC3 oligonucleotide selected from the group consisting of: WV-2111, WV-2113, WV-2114, WV-2148, WV-2149, WV-2152, WV-2153, WV-2156, WV-2157, WV-2387, WV-3069, WV-7523, WV-7524, WV-7525, WV-7526, WV-7527, WV-7528, and any oligonucleotide of any of Formats S40 to S42 of FIG. 1L; or Formats 30-32, 66-69 or 101-103 of FIG. 1. Those skilled in the art, reading the present specification, will appreciate that the present disclosure specifically does not exclude the possibility that any oligonucleotide described herein which is labeled as a hybrid oligonucleotide may also or alternatively operate through another mechanism (e.g., as an antisense oligonucleotide; mediating knock-down via a RNaseH mechanism; sterically hindering translation; or any other biochemical mechanism).

### Chirally Controlled Oligonucleotides and Chirally Controlled Oligonucleotide Compositions

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides are chirally controlled.

The present disclosure provides chirally controlled oligonucleotides, and chirally controlled oligonucleotide compositions which are of high crude purity and of high diastereomeric purity. In some embodiments, the present disclosure provides chirally controlled oligonucleotides, and chirally controlled oligonucleotide compositions which are of high crude purity. In some embodiments, the present disclosure provides chirally controlled oligonucleotides, and chirally controlled oligonucleotide compositions which are of high diastereomeric purity.

In some embodiments, a single-stranded RNAi agent is a substantially pure preparation of an APOC3 oligonucleotide type in that oligonucleotides in the composition that are not of the oligonucleotide type are impurities form the preparation process of said oligonucleotide type, in some case, after certain purification procedures.

In some embodiments, the present disclosure provides oligonucleotides comprising one or more diastereomerically pure internucleotidic linkages with respect to the chiral linkage phosphorus. In some embodiments, the present disclosure provides oligonucleotides comprising one or more diastereomerically pure internucleotidic linkages having the structure of Formula I. In some embodiments, the present disclosure provides oligonucleotides comprising one or more diastereomerically pure internucleotidic linkages with respect to the chiral linkage phosphorus, and one or more phosphate diester linkages. In some embodiments, the present disclosure provides oligonucleotides comprising one or more diastereomerically pure internucleotidic linkages having the structure of Formula I, and one or more phosphate diester linkages. In some embodiments, the present disclosure provides oligonucleotides comprising one or more diastereomerically pure internucleotidic linkages having the structure of Formula I-c, and one or more phosphate diester linkages. In some embodiments, such oligonucleotides are prepared by using stereoselective oligonucleotide synthesis, as described in this application, to form pre-designed diastereomerically pure internucleotidic linkages with respect to the chiral linkage phosphorus. Example internucleotidic linkages, including those having structures of Formula I, are further described below.

In some embodiments, the present disclosure provides a chirally controlled oligonucleotide, wherein at least two of the individual internucleotidic linkages within the oligonucleotide have different stereochemistry and/or different P-modifications relative to one another.

Internucleotidic Linkages

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides comprise any internucleotidic linkage described herein or known in the art.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any internucleotidic linkage described herein or known in the art.

A non-limiting example of an internucleotidic linkage or unmodified internucleotidic linkage is a phosphodiester; non-limiting examples of modified internucleotidic linkages include those in which one or more oxygen of a phosphodiester has been replaced by, as non-limiting examples, sulfur (as in a phosphorothioate), H, alkyl, or another moiety or element which is not oxygen. A non-limiting example of an internucleotidic linkage is a moiety which does not a comprise a phosphorus but serves to link two sugars. A non-limiting example of an internucleotidic linkage is a moiety which does not a comprise a phosphorus but serves to link two sugars in the backbone of an APOC3 oligonucleotide. Disclosed herein are additional non-limiting examples of nucleotides, modified nucleotides, nucleotide analogs, internucleotidic linkages, modified internucleotidic linkages, bases, modified bases, and base analogs, sugars, modified sugars, and sugar analogs, and nucleosides, modified nucleosides, and nucleoside analogs.

In certain embodiments, a internucleotidic linkage has the structure of Formula I:

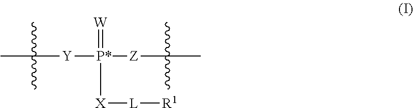

wherein each variable is as defined and described below. In some embodiments, a linkage of Formula I is chiral. In some embodiments, the present disclosure provides a chirally controlled oligonucleotide comprising one or more modified internucleotidic linkages of Formula I. In some embodiments, the present disclosure provides a chirally controlled oligonucleotide comprising one or more modified internucleotidic linkages of Formula I, and wherein individual internucleotidic linkages of Formula I within the oligonucleotide have different P-modifications relative to one another. In some embodiments, the present disclosure provides a chirally controlled oligonucleotide comprising one or more modified internucleotidic linkages of Formula I, and wherein individual internucleotidic linkages of Formula I within the oligonucleotide have different —X-L-R¹ relative to one another. In some embodiments, the present disclosure provides a chirally controlled oligonucleotide comprising one or more modified internucleotidic linkages of Formula I, and wherein individual internucleotidic linkages of Formula I within the oligonucleotide have different X relative to one another. In some embodiments, the present disclosure provides a chirally controlled oligonucleotide comprising one or more modified internucleotidic linkages of Formula I, and wherein individual internucleotidic linkages of Formula I within the oligonucleotide have different -L-R¹ relative to one another. In some embodiments, a chirally controlled oligonucleotide is an APOC3 oligonucleotide in a provided composition that is of the particular oligonucleotide type. In some embodiments, a chirally controlled oligonucleotide is an APOC3 oligonucleotide in a provided composition that has the common base sequence and length, the common pattern of backbone linkages, and the common pattern of backbone chiral centers. In some embodiments, a chirally controlled oligonucleotide is an APOC3 oligonucleotide in a chirally controlled composition that is of the particular oligonucleotide type, and the chirally controlled oligonucleotide is of the type. In some embodiments, a chirally controlled oligonucleotide is an APOC3 oligonucleotide in a provided composition that comprises a predetermined level of a plurality of oligonucleotides that share a common base sequence, a common pattern of backbone linkages, and a common pattern of backbone chiral centers, and the chirally controlled oligonucleotide shares the common base sequence, the common pattern of backbone linkages, and the common pattern of backbone chiral centers.

In some embodiments, a chirally controlled oligonucleotide comprises different internucleotidic phosphorus linkages.

In some embodiments, a phosphorothioate triester linkage comprises a chiral auxiliary, which, for example, is used to control the stereoselectivity of a reaction. In some embodiments, a phosphorothioate triester linkage does not comprise a chiral auxiliary. In some embodiments, a phosphorothioate triester linkage is intentionally maintained until and/or during the administration to a subject.

In some embodiments, a chirally controlled oligonucleotide is linked to a solid support. In some embodiments, a chirally controlled oligonucleotide is cleaved from a solid support.

In some embodiments, a chirally controlled oligonucleotide comprises at least one phosphate diester internucleotidic linkage and at least two consecutive modified internucleotidic linkages. In some embodiments, a chirally controlled oligonucleotide comprises at least one phosphate diester internucleotidic linkage and at least two consecutive phosphorothioate triester internucleotidic linkages.

In some embodiments, the present disclosure provides compositions comprising or consisting of a plurality of provided oligonucleotides (e.g., chirally controlled oligonucleotide compositions). In some embodiments, all such provided oligonucleotides are of the same type, i.e., all have the same base sequence, pattern of backbone linkages (i.e., pattern of internucleotidic linkage types, for example, phosphate, phosphorothioate, etc), pattern of backbone chiral centers (i.e. pattern of linkage phosphorus stereochemistry (Rp/Sp)), and pattern of backbone phosphorus modifications (e.g., pattern of “-XLR¹” groups in Formula I, disclosed herein). In some embodiments, all oligonucleotides of the same type are identical. In many embodiments, however, provided compositions comprise a plurality of oligonucleotides types, typically in pre-determined relative amounts.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any internucleotidic linkage described herein or known in the art. In some embodiments, a moiety that binds ASPGR is, for example, a GalNAc moiety is any GalNAc, or variant or modification thereof, as described herein or known in the art. In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any internucleotidic linkage described herein or known in the art in combination with any other structural element or modification described herein, including but not limited to, base sequence or portion thereof, sugar, base (nucleobase); stereochemistry or pattern thereof; additional chemical moiety, including but not limited to, a targeting moiety, a lipid moiety, a carbohydrate moiety, etc.; seed region; post-seed region; 5′-end structure; 5′-end region; 5′ nucleotide moiety; 3′-end region; 3′-terminal dinucleotide; 3′-end cap; length; additional chemical moiety, including but not limited to, a targeting moiety, lipid moiety, a GalNAc, etc.; format or any structural element thereof, and/or any other structural element or modification described herein; and in some embodiments, the present disclosure pertains to multimers of any such oligonucleotides.

In some embodiments, a chirally controlled oligonucleotide comprises one or more modified internucleotidic phosphorus linkages. In some embodiments, a chirally controlled oligonucleotide comprises, e.g., a phosphorothioate or a phosphorothioate triester linkage.

In some embodiments, a modified internucleotidic linkage is phosphorothioate. In some embodiments, a modified internucleotidic linkage is selected from those described in, for example: US 20110294124, US 20120316224, US 20140194610, US 20150211006, US 20150197540, WO 2015107425, PCT/US2016/043542, and PCT/US2016/043598, Whittaker et al. 2008 Tetrahedron Letters 49: 6984-6987.

Non-limiting examples of internucleotidic linkages also include those described in the art, including, but not limited to, those described in any of: Gryaznov, S.; Chen, J.-K. J. Am. Chem. Soc. 1994, 116, 3143, Jones et al. J. Org. Chem. 1993, 58, 2983, Koshkin et al. 1998 Tetrahedron 54: 3607-3630, Lauritsen et al. 2002 Chem. Comm. 5: 530-531, Lauritsen et al. 2003 Bioo. Med. Chem. Lett. 13: 253-256, Mesmaeker et al. Angew. Chem., Int. Ed. Engl. 1994, 33, 226, Petersen et al. 2003 TRENDS Biotech. 21: 74-81, Schultz et al. 1996 Nucleic Acids Res. 24: 2966, Ts'o et al. Ann. N. Y. Acad. Sci. 1988, 507, 220, and Vasseur et al. J. Am. Chem. Soc. 1992, 114, 4006.

In some embodiments, the present disclosure provides a chirally controlled oligonucleotide comprising a sequence found in any oligonucleotide disclosed herein. In some embodiments, the present disclosure provides a chirally controlled oligonucleotide comprising a sequence found in any oligonucleotide disclosed herein, wherein one or more U is replaced with T. In some embodiments, the present disclosure provides a chirally controlled oligonucleotide comprising a sequence found in any oligonucleotide disclosed herein, wherein the said sequence has over 50% identity with the sequence of any oligonucleotide disclosed herein.

In some embodiments, the present disclosure provides a chirally controlled oligonucleotide comprising the sequence of any oligonucleotide disclosed herein. In some embodiments, the present disclosure provides a chirally controlled oligonucleotide having the sequence of any oligonucleotide disclosed herein. In some embodiments, the present disclosure provides a chirally controlled oligonucleotide comprising a sequence found in any oligonucleotide disclosed herein, wherein the oligonucleotides have a pattern of backbone linkages, pattern of backbone chiral centers, and/or pattern of backbone phosphorus modifications described herein.

In some embodiments, the present disclosure provides a chirally controlled oligonucleotide comprising a sequence found in any oligonucleotide disclosed herein. In some embodiments, the present disclosure provides a chirally controlled oligonucleotide comprising a sequence found in any oligonucleotide disclosed herein, wherein one or more T is substituted with U. In some embodiments, the present disclosure provides a chirally controlled oligonucleotide comprising a sequence found in any oligonucleotide disclosed herein, wherein the said sequence has over 50% identity with the sequence of any oligonucleotide disclosed herein. In some embodiments, the present disclosure provides a chirally controlled oligonucleotide comprising a sequence found in any oligonucleotide disclosed herein, wherein the said sequence has over 60% identity with the sequence of any oligonucleotide disclosed herein. In some embodiments, the present disclosure provides a chirally controlled oligonucleotide comprising a sequence found in any oligonucleotide disclosed herein, wherein the said sequence has over 70% identity with the sequence of any oligonucleotide disclosed herein. In some embodiments, the present disclosure provides a chirally controlled oligonucleotide comprising a sequence found in any oligonucleotide disclosed herein, wherein the said sequence has over 80% identity with the sequence of any oligonucleotide disclosed herein. In some embodiments, the present disclosure provides a chirally controlled oligonucleotide comprising a sequence found in any oligonucleotide disclosed herein, wherein the said sequence has over 90% identity with the sequence of any oligonucleotide disclosed herein. In some embodiments, the present disclosure provides a chirally controlled oligonucleotide comprising a sequence found in any oligonucleotide disclosed herein, wherein the said sequence has over 95% identity with the sequence of any oligonucleotide disclosed herein. In some embodiments, the present disclosure provides a chirally controlled oligonucleotide comprising the sequence of any oligonucleotide disclosed herein. In some embodiments, the present disclosure provides a chirally controlled oligonucleotide having the sequence of any oligonucleotide disclosed herein.

In some embodiments, the present disclosure provides a chirally controlled oligonucleotide comprising the sequence of any oligonucleotide disclosed herein, wherein at least one internucleotidic linkage is

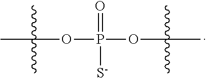 [SMILES: *OP(=O)([S-])O*]

In some embodiments, the present disclosure provides a chirally controlled oligonucleotide comprising the sequence of any oligonucleotide disclosed herein, wherein each internucleotidic linkage is

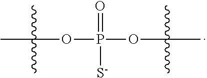 [SMILES: *OP(=O)([S-])O*]

In some embodiments, the present disclosure provides a chirally controlled oligonucleotide having the sequence of any oligonucleotide disclosed herein, wherein at least one internucleotidic linkage is

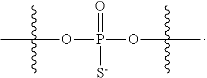 [SMILES: *OP(=O)([S-])O*]

In some embodiments, the present disclosure provides a chirally controlled oligonucleotide having the sequence of any oligonucleotide disclosed herein, wherein each internucleotidic linkage is

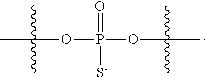 [SMILES: *OP(=O)([S-])O*]

In some embodiments, the present disclosure provides a chirally controlled oligonucleotide having the sequence of any oligonucleotide disclosed herein, wherein each cytosine is optionally and independently replaced by 5-methylcytosine. In some embodiments, the present disclosure provides a chirally controlled oligonucleotide having the sequence of any oligonucleotide disclosed herein, wherein at least one cytosine is optionally and independently replaced by 5-methylcytosine. In some embodiments, the present disclosure provides a chirally controlled oligonucleotide having the sequence of any oligonucleotide disclosed herein, wherein each cytosine is optionally and independently replaced by 5-methylcytosine.

### Bases (Nucleobases)

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides comprise any nucleobase described herein or known in the art.

In some embodiments, a nucleobase present in a provided oligonucleotide is a natural nucleobase or a modified nucleobase derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Example modified nucleobases are disclosed in Chiu and Rana, RNA, 2003, 9, 1034-1048, Limbach et al. Nucleic Acids Research, 1994, 22, 2183-2196 and Revankar and Rao, Comprehensive Natural Products Chemistry, vol. 7, 313. In some embodiments, a modified nucleobase is substituted uracil, thymine, adenine, cytosine, or guanine. In some embodiments, a modified nucleobase is a functional replacement, e.g., in terms of hydrogen bonding and/or base pairing, of uracil, thymine, adenine, cytosine, or guanine. In some embodiments, a nucleobase is optionally substituted uracil, thymine, adenine, cytosine, 5-methylcytosine, or guanine. In some embodiments, a nucleobase is uracil, thymine, adenine, cytosine, 5-methylcytosine, or guanine.

In some embodiments, a modified base is optionally substituted adenine, cytosine, guanine, thymine, or uracil. In some embodiments, a modified nucleobase is independently adenine, cytosine, guanine, thymine or uracil, modified by one or more modifications by which:

(1) a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof;

(2) one or more atoms of a nucleobase are independently replaced with a different atom selected from carbon, nitrogen and sulfur;

(3) one or more double bonds in a nucleobase are independently hydrogenated; or

(4) one or more aryl or heteroaryl rings are independently inserted into a nucleobase.

Various additional nucleobases are described in the art. Sugars

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides comprise any sugar described herein or known in the art.

In some embodiments, provided oligonucleotides capable of directing single-stranded RNA interference comprise one or more modified sugar moieties beside the natural sugar moieties.

The most common naturally occurring nucleotides are comprised of ribose sugars linked to the nucleobases adenosine (A), cytosine (C), guanine (G), and thymine (T) or uracil (U). Also contemplated are modified nucleotides wherein a phosphate group or linkage phosphorus in the nucleotides can be linked to various positions of a sugar or modified sugar. As non-limiting examples, the phosphate group or linkage phosphorus can be linked to the 2″, 3″, 4″ or 5″ hydroxyl moiety of a sugar or modified sugar. Nucleotides that incorporate modified nucleobases as described herein are also contemplated in this context. In some embodiments, nucleotides or modified nucleotides comprising an unprotected —OH moiety are used in accordance with methods of the present disclosure.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any base (nucleobase), modified base or base analog described herein or known in the art. In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any base described herein or known in the art in combination with any other structural element or modification described herein, including but not limited to, base sequence or portion thereof, sugar; internucleotidic linkage; stereochemistry or pattern thereof; additional chemical moiety, including but not limited to, a targeting moiety, lipid moiety, a GalNAc moiety, etc.; 5′-end structure; 5′-end region; 5′ nucleotide moiety; seed region; post-seed region; 3′-end region; 3′-terminal dinucleotide; 3′-end cap; pattern of modifications of sugars, bases or internucleotidic linkages; format or any structural element thereof, and/or any other structural element or modification described herein; and in some embodiments, the present disclosure pertains to multimers of any such oligonucleotides.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any sugar.

Various additional sugars are described in the art.

Base Sequence of an APOC3 Oligonucleotide

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides can comprise any base sequence or portion thereof, described herein, wherein a portion is a span of at least 15 contiguous bases, or a span of at least 15 contiguous bases with 1-5 mismatches.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any base sequence described herein. In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any base sequence or portion thereof, described herein. In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any base sequence or portion thereof, described herein, wherein a portion is a span of 15 contiguous bases, or a span of 15 contiguous bases with 1-5 mismatches.

The sequence of a single-stranded RNAi agent has a sufficient length and identity to a transcript target to mediate target-specific RNA interference. In some embodiments, the RNAi agent is complementary to a portion of a transcript target sequence.

The base sequence of a single-stranded RNAi agent is complementary to that of a target transcript. As used herein, “target transcript sequence,” “target sequence”, “target gene”, and the like, refer to a contiguous portion of the nucleotide sequence of an mRNA molecule formed during the transcription of a gene, e.g., a target gene, including mRNA that is a product of RNA processing of a primary transcription product.

The terms “complementary,” “fully complementary” and “substantially complementary” herein may be used with respect to the base matching between the strand of a single-stranded RNAi agent and a target sequence or between an antisense oligonucleotide and a target sequence, as will be understood from the context of their use. A strand of a single-stranded RNAi agent or antisense oligonucleotide or other oligonucleotide is complementary to that of a target sequence when each base of the single-stranded RNAi agent, antisense oligonucleotide or other oligonucleotide is capable of base-pairing with a sequential base on the target strand, when maximally aligned. As a non-limiting example, if a target sequence has, for example, a base sequence of 5′-GCAUAGCGAGCGAGGGAAAAC-3′ (SEQ ID NO: 1), an APOC3 oligonucleotide with a base sequence of 5′GUUUUCCCUCGCUCGCUAUGC-3′ (SEQ ID NO: 2) is complementary or fully complementary to such a target sequence. It is noted, of course, that substitution of T for U, or vice versa, does not alter the amount of complementarity.

As used herein, a polynucleotide that is “substantially complementary” to a target sequence is largely or mostly complementary but not 100% complementary. In some embodiments, a sequence (e.g., a strand of a single-stranded RNAi agent or an antisense oligonucleotide) which is substantially complementary has 1, 2, 3, 4 or 5 mismatches from a sequence which is 100% complementary to the target sequence. In the case of a single-stranded RNAi agent, this disclosure notes that the 5′ terminal nucleotide (N1) in many cases has a mismatch from the complement of a target sequence. Similarly, in a single-stranded RNAi agent, the 3′-terminal dinucleotide, if present, can be a mismatch from the complement of the target sequence. As a non-limiting example, if a target sequence has, for example, a base sequence of 5′-GCAUAGCGAGCGAGGGAAAAC-3′ (SEQ ID NO: 3), a single-stranded RNAi agent with a base sequence of 5′TUUUUCCCUCGCUCGCUAUTU-3′ (SEQ ID NO: 4) is substantially complementary to such a target sequence.

The present disclosure presents, in Table 1A and elsewhere, various single-stranded RNAi agents and antisense oligonucleotides and other oligonucleotides, each of which has a defined base sequence. In some embodiments, the disclosure encompasses any oligonucleotide having a base sequence which is, comprises, or comprises a portion of the base sequence of any various single-stranded RNAi agent, antisense oligonucleotide and other oligonucleotide disclosed herein. In some embodiments, the disclosure encompasses any oligonucleotide having a base sequence which is, comprises, or comprises a portion of the base sequence of any various single-stranded RNAi agent, antisense oligonucleotide and other oligonucleotide disclosed herein, which has any chemical modification, stereochemistry, format, structural feature (e.g., if the oligonucleotide is a single-stranded RNAi agent, the 5′-end structure, 5′-end region, 5′ nucleotide moiety, seed region, post-seed region, 3′-end region, 3′-terminal dinucleotide, 3′-end cap, or any structure, pattern or portion thereof), and/or any other modification described herein (e.g., conjugation with another moiety, such as a targeting moiety, carbohydrate moiety, a GalNAc moiety, lipid moiety, etc.; and/or multimerization).

In some embodiments, an APOC3 oligonucleotide has a base sequence which is, comprises or comprises a portion of the base sequence of any oligonucleotide disclosed herein.

In some embodiments, the present disclosure discloses an APOC3 oligonucleotide of a sequence recited herein. In some embodiments, the present disclosure discloses an APOC3 oligonucleotide of a sequence recited herein, wherein the oligonucleotide is capable of directing a decrease in the expression and/or level of a target gene or its gene product. In some embodiments, an APOC3 oligonucleotide of a recited sequence is a single-stranded RNAi agent. In some embodiments, an APOC3 oligonucleotide of a recited sequence is an antisense oligonucleotide which directs RNase H-mediated knockdown. In some embodiments, an APOC3 oligonucleotide of a recited sequence directs both RNA interference and RNase H-mediated knockdown. In some embodiments, an APOC3 oligonucleotide of a recited sequence comprises any structure described herein (e.g., any 5′-end structure, 5′-end region, 5′ nucleotide moiety, seed region, post-seed region, 3′-terminal dinucleotide, 3′-end cap, or any portion of any of these structures, or any chemistry, stereochemistry, additional chemical moiety, etc., described herein). If the oligonucleotide is a ssRNAi agent, the sequence can be preceded by a T (as a non-limiting example, a 2′-deoxy T, 5′-(R)-Me OH T, 5′-(R)-Me PO T, 5′-(R)-Me PS T, 5′-(R)-Me PH T, 5′-(S)-Me OH T, 5′-(S)-Me PO T, 5′-(S)-Me PS T, or 5′-(S)—PH T) or the first nucleobase is replaced by a T (as a non-limiting example, a 2′-deoxy T, 5′-(R)-Me OH T, 5′-(R)-Me PO T, 5′-(R)-Me PS T, 5′-(R)-Me PH T, 5′-(S)-Me OH T, 5′-(S)-Me PO T, 5′-(S)-Me PS T, or 5′-(S)—PH T) and/or followed by a 3′-terminal dinucleotide (e.g., as non-limiting examples: TT, UU, TU, etc.). In various sequences, U can be replaced by T or vice versa, or a sequence can comprise a mixture of U and T. In some embodiments, an APOC3 oligonucleotide has a length of no more than about 49, 45, 40, 30, 35, 25, 23 total nucleotides. In some embodiments, a portion is a span of at least 15, 16, 17, 18, 19, 20, 21, 22, 23, 24, or 25 total nucleotides with 0-3 mismatches. In some embodiments, a portion is a span of at least 15, 16, 17, 18, 19, 20, 21, 22, 23, 24, or 25 total nucleotides with 0-3 mismatches, wherein a span with 0 mismatches is complementary and a span with 1 or more mismatches is a non-limiting example of substantial complementarity. In some embodiments, wherein the sequence recited above starts with a U at the 5′-end, the U can be deleted and/or replaced by another base. In some embodiments, the disclosure encompasses any oligonucleotide having a base sequence which is or comprises or comprises a portion of the base sequence of any oligonucleotide disclosed herein, which has a format or a portion of a format disclosed herein.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any base sequence described herein. In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any base sequence or portion thereof, described herein. In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any base sequence or portion thereof, described herein, wherein a portion is a span of 15 contiguous bases, or a span of 15 contiguous bases with 1-5 mismatches. In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any base sequence or portion thereof described herein in combination with any other structural element or modification described herein, including but not limited to, sugar, base; internucleotidic linkage; stereochemistry or pattern thereof; additional chemical moiety, including but not limited to, a targeting moiety, lipid moiety, a GalNAc moiety, etc.; 5′-end structure; 5′-end region; 5′ nucleotide moiety; seed region; post-seed region; 3′-end region; 3′-terminal dinucleotide; 3′-end cap; pattern of modifications of sugars, bases or internucleotidic linkages; format or any structural element thereof, and/or any other structural element or modification described herein; and in some embodiments, the present disclosure pertains to multimers of any such oligonucleotides.

Non-limiting examples of oligonucleotides having various base sequences are disclosed in Table 1A, below.

TABLE 1A
Oligonucleotides.
APOC3 oligonucleotides.

| | | | | SEQ |
|---|---|---|---|---|
| | | | | ID |
| ID | Naked Sequence | Sequence | Stereochemistry | NO: |
| WV- | AAUACUGUCCCUUUUAAG | rA rA rU rA rC rU rG rU rC rC rC rU rU rU rU rA rA | OOOOOOOOOOOOOOOO | 5 |
| 1161 | CUU | rG rCmUmU | OOOO | |
| WV- | AAUACUGUCCCUUUUAAG | rA * rA rU rA rC rU rG rU rC rC rC rU rU rU rU rA | XOOOOOOOOOOOOOOO | 6 |
| 1162 | CUU | rA rG rCmUmU | OOOO | |
| WV- | AAUACUGUCCCUUUUAAG | rA rA * rU rA rC rU rG rU rC rC rC rU rU rU rU rA | OXOOOOOOOOOOOOOO | 7 |
| 1163 | CUU | rA rG rCmUmU | OOOO | |
| WV- | AAUACUGUCCCUUUUAAG | rA rA rU * rA rC rU rG rU rC rC rC rU rU rU rU rA | OOXOOOOOOOOOOOOO | 8 |
| 1164 | CUU | rA rG rCmUmU | OOOO | |
| WV- | AAUACUGUCCCUUUUAAG | rA rA rU rA * rC rU rG rU rC rC rC rU rU rU rU rA | OOOXOOOOOOOOOOOO | 9 |
| 1165 | CUU | rA rG rCmUmU | OOOO | |
| WV- | AAUACUGUCCCUUUUAAG | rA rA rU rA rC * rU rG rU rC rC rC rU rU rU rU rA | OOOOXOOOOOOOOOOO | 10 |
| 1166 | CUU | rA rG rCmUmU | OOOO | |
| WV- | AAUACUGUCCCUUUUAAG | rA rA rU rA rC rU * rG rU rC rC rC rU rU rU rU rA | OOOOOXOOOOOOOOOO | 11 |
| 1167 | CUU | rA rG rCmUmU | OOOO | |
| WV- | AAUACUGUCCCUUUUAAG | rA rA rU rA rC rU rG * rU rC rC rC rU rU rU rU rA | OOOOOOXOOOOOOOOO | 12 |
| 1168 | CUU | rA rG rCmUmU | OOOO | |
| WV- | AAUACUGUCCCUUUUAAG | rA rA rU rA rC rU rG rU * rC rC rC rU rU rU rU rA | OOOOOOOXOOOOOOOO | 13 |
| 1169 | CUU | rA rG rCmUmU | OOOO | |
| WV- | AAUACUGUCCCUUUUAAG | rA rA rU rA rC rU rG rU rC * rC rC rU rU rU rU rA | OOOOOOOOXOOOOOOO | 14 |
| 1170 | CUU | rA rG rCmUmU | OOOO | |
| WV- | AAUACUGUCCCUUUUAAG | rA rA rU rA rC rU rG rU rC rC * rC rU rU rU rU rA | OOOOOOOOOXOOOOOO | 15 |
| 1171 | CUU | rA rG rCmUmU | OOOO | |
| WV- | AAUACUGUCCCUUUUAAG | rA rA rU rA rC rU rG rU rC rC rC * rU rU rU rU rA | OOOOOOOOOOOXOOOO | 16 |
| 1172 | CUU | rA rG rCmUmU | OOOO | |
| WV- | AAUACUGUCCCUUUUAAG | rA rA rU rA rC rU rG rU rC rC rC rU * rU rU rU rA | OOOOOOOOOOOXOOOO | 17 |
| 1173 | CUU | rA rG rCmUmU | OOOO | |
| WV- | AAUACUGUCCCUUUUAAG | rA rA rU rA rC rU rG rU rC rC rC rU rU * rU rU rA | OOOOOOOOOOOOXOOO | 18 |
| 1174 | CUU | rA rG rCmUmU | OOOO | |
| WV- | AAUACUGUCCCUUUUAAG | rA rA rU rA rC rU rG rU rC rC rC rU rU rU * rU rA | OOOOOOOOOOOOOXOO | 19 |
| 1175 | CUU | rA rG rCmUmU | OOOO | |
| WV- | AAUACUGUCCCUUUUAAG | rA rA rU rA rC rU rG rU rC rC rC rU rU rU rU * rA | OOOOOOOOOOOOOOXO | 20 |
| 1176 | CUU | rA rG rCmUmU | OOOO | |
| WV- | AAUACUGUCCCUUUUAAG | rA rA rU rA rC rU rG rU rC rC rC rU rU rU rU rA * | OOOOOOOOOOOOOOOX | 21 |
| 1177 | CUU | rA rG rCmUmU | OOOO | |
| WV- | AAUACUGUCCCUUUUAAG | rA rA rU rA rC rU rG rU rC rC rC rU rU rU rU rA rA | OOOOOOOOOOOOOOOO | 22 |
| 1178 | CUU | * rG rCmUmU | XOOO | |
| WV- | AAUACUGUCCCUUUUAAG | rA rA rU rA rC rU rG rU rC rC rC rU rU rU rU rA rA | OOOOOOOOOOOOOOOO | 23 |
| 1179 | CUU | rG * rCmUmU | OXOO | |
| WV- | AAUACUGUCCCUUUUAAG | rA rA rU rA rC rU rG rU rC rC rC rU rU rU rU rA rA | OOOOOOOOOOOOOOOO | 24 |
| 1180 | CUU | rG rC * mUmU | OOXO | |
| WV- | AAUACUGUCCCUUUUAAG | rA rA rU rA rC rU rG rU rC rC rC rU rU rU rU rA rA | OOOOOOOOOOOOOOOO | 25 |
| 1181 | CUU | rG rCmU * mU | OOOX | |
| WV- | GCUUAAAAGGGACAGUAU | rG rC rU rU rA rA rA rA rG rG rG rA rC rA rG rU rA | OOOOOOOOOOOOOOOO | 26 |
| 1182 | UUU | rU rUmUmU | OOOO | |
| WV- | GUGCAUCCUUGGCGGUCU | PO fGmU fGmC fAmU fCmC fUmU fGmG fCmG | OOOOOOOOOOOOOOOO | 27 |
| 1237 | UUU | fGmU fCmU fUmUmU | OOOO | |
| WV- | GUGCAUCCUUGGCGGUCU | PO fG * mU fG * mC fA * mU fC * mC fU * mU fG | XOXOXOXOXOXOXOXOXO | 28 |
| 1238 | UUU | * mG fC * mG fG * mU fC * mU fU * mUmU | XO | |
| WV- | GUGCAUCCUUGGCGGUCU | PO rG fU rG fC rA fU fC fC fU fU rG rG fC rG rG fU | OOOOOOOOOOOOOOOO | 29 |
| 1239 | UUU | fC fU fUmUmU | OOOO | |
| WV- | GUGCAUCCUUGGCGGUCU | PO fGmU fGmC fAmU fCmC fUmU fGmG fCmG * | OOOOOOOOOOOOOXXXX | 30 |
| 1240 | UUU | fG * mU * fC * mU * fU * mU * mU | XXX | |
| WV- | GUCCCAGGUGGCCCAGCA | rG rU rC rC rC rA rG rG rU rG rG rC rC rC rA rG rC | OOOOOOOOOOOOOOOO | 31 |
| 1241 | GUU | rA rGmUmU | OOOO | |
| WV- | CUGCUGGGCCACCUGGGA | PO fCmU fGmC fUmG fGmG fCmC fAmC fCmU | OOOOOOOOOOOOOOOO | 32 |
| 1242 | CUU | fGmG fGmA fCmUmU | OOOO | |
| WV- | CUGCUGGGCCACCUGGGA | PO fC * mU fG * mC fU * mG fG * mG fC * mC fA | XOXOXOXOXOXOXOXOXO | 33 |
| 1243 | CUU | * mC fC * mU fG * mG fG * mA fC * mUmU | XO | |
| WV- | CUGCUGGGCCACCUGGGA | PO fC fU rG fC fU rG rG rG fC fC rA fC fC fU rG rG | OOOOOOOOOOOOOOOO | 34 |
| 1244 | CUU | rG rA fCmUmU | OOOO | |
| WV- | CUGCUGGGCCACCUGGGA | PO fCmU fGmC fUmG fGmG fCmC fAmC fCmU * | OOOOOOOOOOOOOXXXX | 35 |
| 1245 | CUU | fG * mG * fG * mA * fC * mU * mU | XXX | |
| WV- | GACCCUGAGGUCAGACCA | rG rA rC rC rC rU rG rA rG rG rU rC rA rG rA rC rC | OOOOOOOOOOOOOOOO | 36 |
| 1246 | AUU | rA rAmUmU | OOOO | |
| WV- | UUGGUCUGACCUCAGGGU | PO rU rU rG rG rU rC rU rG rA rC rC rU rC rA rG | OOOOOOOOOOOOOOOO | 37 |
| 1247 | CUU | rG rG rU rCmUmU | OOOO | |
| WV- | UUGGUCUGACCUCAGGGU | PO fUmU fGmG fUmC fUmG fAmC fCmU fCmA | OOOOOOOOOOOOOOOO | 38 |
| 1248 | CUU | fGmG fGmU fCmUmU | OOOO | |
| WV- | UUGGUCUGACCUCAGGGU | PO fU * mU fG * mG fU * mC fU * mG fA * mC fC | XOXOXOXOXOXOXOXOXO | 39 |
| 1249 | CUU | * mU fC * mA fG * mG fG * mU fC * mUmU | XO | |
| WV- | UUGGUCUGACCUCAGGGU | PO fU * mU * fG * mG * fU * mC * fU * mG * fA | XXXXXXXXXXXXXXXXXXXX | 40 |
| 1250 | CUU | * mC * fC * mU * fC * mA * fG * mG * fG * mU * | | |
| | | fC * mU * mU | | |
| WV- | UUGGUCUGACCUCAGGGU | PO fU fU rG rG fU fC fU rG rA fC fC fU fC rA rG rG | OOOOOOOOOOOOOOOO | 41 |
| 1251 | CUU | rG fU fCmUmU | OOOO | |
| WV- | UUGGUCUGACCUCAGGGU | PO fUmU fGmG fUmC fUmG fAmC fCmU fCmA * | OOOOOOOOOOOOOXXXX | 42 |
| 1252 | CUU | fG * mG * fG * mU * fC * mU * mU | XXX | |
| WV- | CCUCCAGGCAUGCUGGCC | rC rC rU rC rC rA rG rG rC rA rU rG rC rU rG rG rC | OOOOOOOOOOOOOOOO | 43 |
| 1253 | UUU | rC rUmUmU | OOOO | |
| WV- | AGGCCAGCAUGCCUGGAG | PO fAmG fGmC fCmA fGmC fAmU fGmC fCmU | OOOOOOOOOOOOOOOO | 44 |
| 1254 | GUU | fGmG fAmG fGmUmU | OOOO | |
| WV- | AGGCCAGCAUGCCUGGAG | PO fA * mG fG * mC fC * mA fG * mC fA * mU fG | XOXOXOXOXOXOXOXOXO | 45 |
| 1255 | GUU | * mC fC * mU fG * mG fA * mG fG * mUmU | XO | |
| WV- | AGGCCAGCAUGCCUGGAG | PO rA rG rG fC fC rA rG fC rA fU rG fC fC fU rG rG | OOOOOOOOOOOOOOOO | 46 |
| 1256 | GUU | rA rG rGmUmU | OOOO | |
| WV- | AGGCCAGCAUGCCUGGAG | PO fAmG fGmC fCmA fGmC fAmU fGmC fCmU * | OOOOOOOOOOOOOXXXX | 47 |
| 1257 | GUU | fG * mG * fA * mG * fG * mU * mU | XXX | |
| WV- | GUGCAGGAGUCCCAGGUG | rG rU rG rC rA rG rG rA rG rU rC rC rC rA rG rG | OOOOOOOOOOOOOOOO | 48 |
| 1258 | GUU | rU rG rGmUmU | OOOO | |
| WV- | CCACCUGGGACUCCUGCA | PO fCmC fAmC fCmU fGmG fGmA fCmU fCmC | OOOOOOOOOOOOOOOO | 49 |
| 1259 | CUU | fUmG fCmA fCmUmU | OOOO | |
| WV- | CCACCUGGGACUCCUGCA | PO fC * mC fA * mC fC * mU fG * mG fG * mA fC | XOXOXOXOXOXOXOXOXO | 50 |
| 1260 | CUU | * mU fC * mC fU * mG fC * mAfC * mUmU | XO | |
| WV- | CCACCUGGGACUCCUGCA | PO fC fC rA fC fC fU rG rG rG rA fC fU fC fC fU rG | OOOOOOOOOOOOOOOO | 51 |
| 1261 | CUU | fC rA fCmUmU | OOOO | |
| WV- | CCACCUGGGACUCCUGCA | PO fCmC fAmC fCmU fGmG fGmA fCmU fCmC * | OOOOOOOOOOOOOXXXX | 52 |
| 1262 | CUU | fU * mG * fC * mA * fC * mU * mU | XXX | |
| WV- | GGGCUGGGUGACCGAUG | rG rG rG rC rU rG rG rG rU rG rA rC rC rG rA rU | OOOOOOOOOOOOOOOO | 53 |
| 1263 | GCUU | rG rG rCmUmU | OOOO | |
| WV- | GCCAUCGGUCACCCAGCCC | PO fGmC fCmA fUmC fGmG fUmC fAmC fCmC | OOOOOOOOOOOOOOOO | 54 |
| 1264 | UU | fAmG fCmC fCmUmU | OOOO | |
| WV- | GCCAUCGGUCACCCAGCCC | PO fG * mC fC * mA fU * mC fG * mG fU * mC fA | XOXOXOXOXOXOXOXOXO | 55 |
| 1265 | UU | * mC fC * mC fA * mG fC * mC fC * mUmU | XO | |
| WV- | GCCAUCGGUCACCCAGCCC | PO rG fC fC rA fU fC rG rG fU fC rA fC fC fC rA rG | OOOOOOOOOOOOOOOO | 56 |
| 1266 | UU | fC fC fCmUmU | OOOO | |
| WV- | GCCAUCGGUCACCCAGCCC | PO fGmC fCmA fUmC fGmG fUmC fAmC fCmC * | OOOOOOOOOOOOOXXXX | 57 |
| 1267 | UU | fA * mG * fC * mC * fC * mU * mU | XXX | |
| WV- | GCCUCCCAAUAAAGCUGG | rG rC rC rU rC rC rC rA rA rU rA rA rA rG rC rU rG | OOOOOOOOOOOOOOO | 58 |
| 1268 | AUU | rG rAmUmU | OOOO | |
| WV- | UCCAGCUUUAUUGGGAG | PO fUmC fCmA fGmC fUmU fUmA fUmU fGmG | OOOOOOOOOOOOOOOO | 59 |
| 1269 | GCUU | fGmA fGmG fCmUmU | OOOO | |
| WV- | UCCAGCUUUAUUGGGAG | PO fU * mC fC * mA fG * mC fU * mU fU * mA fU | XOXOXOXOXOXOXOXOXO | 60 |
| 1270 | GCUU | * mU fG * mG fG * mA fG * mG fC * mUmU | XO | |
| WV- | UCCAGCUUUAUUGGGAG | PO fU fC fC rA rG fC fU fU fU rA fU fU rG rG rG rA | OOOOOOOOOOOOOOOO | 61 |
| 1271 | GCUU | rG rG fCmUmU | OOOO | |
| WV- | UCCAGCUUUAUUGGGAG | PO fUmC fCmA fGmC fUmU fUmA fUmU fGmG * | OOOOOOOOOOOOOXXXX | 62 |
| 1272 | GCUU | fG * mA * fG * mG * fC * mU * mU | XXX | |
| WV- | CCGUGGCUGCCUGAGACC | rC rC rG rU rG rG rC rU rG rC rC rU rG rA rG rA rC | OOOOOOOOOOOOOOOO | 63 |
| 1273 | UUU | rC rUmUmU | OOOO | |
| WV- | AGGUCUCAGGCAGCCACG | PO fAmG fGmU fCmU fCmA fGmG fCmA fGmC | OOOOOOOOOOOOOOOO | 64 |
| 1274 | GUU | fCmA fCmG fGmUmU | OOOO | |
| WV- | AGGUCUCAGGCAGCCACG | PO fA * mG fG * mU fC * mU fC * mA fG * mG fC | XOXOXOXOXOXOXOXOXO | 65 |
| 1275 | GUU | * mA fG * mC fC * mA fC * mG fG * mUmU | XO | |
| WV- | AGGUCUCAGGCAGCCACG | PO rA rG rG fU fC fU fC rA rG rG fC rA rG fC fC rA | OOOOOOOOOOOOOOOO | 66 |
| 1276 | GUU | fC rG rGmUmU | OOOO | |
| WV- | AGGUCUCAGGCAGCCACG | PO fAmG fGmU fCmU fCmA fGmG fCmA fGmC * | OOOOOOOOOOOOOXXXX | 67 |
| 1277 | GUU | fC * mA * fC * mG * fG * mU * mU | XXX | |
| WV- | GCUUAAAAGGGACAGUAU | rG rC rU rU rA rA rA rA rG rG rG rA rC rA rG rU rA | OOOOOOOOOOOOOOOO | 68 |
| 1278 | UUU | rU rUmUmU | OOOO | |
| WV- | AAUACUGUCCCUUUUAAG | PO rA rA rU rA rC rU rG rU rC rC rC rU rU rU rU rA | OOOOOOOOOOOOOOOO | 69 |
| 1279 | CUU | rA rG rCmUmU | OOOO | |
| WV- | AAUACUGUCCCUUUUAAG | PO fAmA fUmA fCmU fGmU fCmC fCmU fUmU | OOOOOOOOOOOOOOOO | 70 |
| 1280 | CUU | fUmA fAmG fCmUmU | OOOO | |
| WV- | AAUACUGUCCCUUUUAAG | PO fA * mA fU * mA fC * mU fG * mU fC * mC fC | XOXOXOXOXOXOXOXOXO | 71 |
| 1281 | CUU | * mU fU * mU fU * mA fA * mG fC * mUmU | XO | |
| WV- | AAUACUGUCCCUUUUAAG | PO fA * mA * fU * mA * fC * mU * fG * mU * fC * | XXXXXXXXXXXXXXXXXXXX | 72 |
| 1282 | CUU | mC * fC * mU * fU * mU * fU * mA * fA * mG * | | |
| | | fC * mU * mU | | |
| WV- | AAUACUGUCCCUUUUAAG | PO rA rA fU rA fC fU rG fU fC fC fC fU fU fU fU rA | OOOOOOOOOOOOOOOO | 73 |
| 1283 | CUU | rA rG fCmUmU | OOOO | |
| WV- | AAUACUGUCCCUUUUAAG | PO fAmA fUmA fCmU fGmU fCmC fCmU fUmU * | OOOOOOOOOOOOOXXXX | 74 |
| 1284 | CUU | fU * mA * fA * mG * fC * mU * mU | XXX | |
| WV- | GGGACAGTATTCTCAGTGA | rG rG rG rA rC rA rG rT rA rT rT rC rT rC rA rG rT | OOOOOOOOOOOOOOOO | 75 |
| 1285 | UU | rG rAmUmU | OOOO | |
| WV- | UCACUGAGAAUACUGUCC | PO rU rC rA rC rU rG rA rG rA rA rU rA rC rU rG rU | OOOOOOOOOOOOOOOO | 76 |
| 1286 | CUU | rC rC rCmUmU | OOOO | |
| WV- | UCACUGAGAAUACUGUCC | PO fUmC fAmC fUmG fAmG fAmA fUmA fCmU | OOOOOOOOOOOOOOOO | 77 |
| 1287 | CUU | fGmU fCmC fCmUmU | OOOO | |
| WV- | UCACUGAGAAUACUGUCC | PO fU * mC fA * mC fU * mG fA * mG fA * mA fU | XOXOXOXOXOXOXOXOXO | 78 |
| 1288 | CUU | * mA fC * mU fG * mU fC * mC fC * mUmU | XO | |
| WV- | UCACUGAGAAUACUGUCC | PO fU * mC * fAmC * fUmG * fAmG * fAmA * | XXOXOXOXOXOXOXOXOX | 79 |
| 1289 | CUU | fUmA * fCmU * fGmU * fCmC * fCmU * mU | OX | |
| WV- | UCACUGAGAAUACUGUCC | PO fU * mC * fA * mC * fU * mG * fA * mG * fA * | XXXXXXXXXXXXXXXXXXXX | 80 |
| 1290 | CUU | mA * fU * mA * fC * mU * fG * mU * fC * mC * fC | | |
| | | * mU * mU | | |
| WV- | UCACUGAGAAUACUGUCC | PO fU fC rA fC rU rG rA rG rA rA fU rA fC fU rG fU | OOOOOOOOOOOOOOOO | 81 |
| 1291 | CUU | fC fC fCmUmU | OOOO | |
| WV- | UCACUGAGAAUACUGUCC | PO fU * mC fAmC fUmG fAmG fAmA fUmA fCmU | XOOOOOOOOOOOXXXX | 82 |
| 1292 | CUU | * fG * mU * fC * mC * fC * mU * mU | XXX | |
| WV- | UCACUGAGAAUACUGUCC | PO fUmC fAmC fUmG fAmG fAmA fUmA fCmU * | OOOOOOOOOOOOOXXXX | 83 |
| 1293 | CUU | fG * mU * fC * mC * fC * mU * mU | XXX | |
| WV- | UCACUGAGAAUACUGUCC | PO fU * mC * fAmC fUmG fAmG fAmA fUmA | XXOOOOOOOOOOOXXXXX | 84 |
| 1294 | CUU | fCmU * fG * mU * fC * mC * fC * mU * mU | XX | |
| WV- | UCACUGAGAAUACUGUCC | PO fU * mC * fAmC * fUmG * fAmG * fAmA * | XXOXOXOXOXOXOXXXXXX | 85 |
| 1295 | CUU | fUmA * fCmU * fG * mU * fC * mC * fC * mU * | X | |
| | | mU | | |
| WV- | UCACUGAGAAUACUGUCC | POmU * fC * mA fC * mU fG * mA fG * mA fA * | XXOXOXOXOXOXOXXXXXX | 86 |
| 1296 | CUU | mU fA * mC fU * mG * fU * mC * fC * mC * | O | |
| | | mUmU | | |
| WV- | AAAGCUGGACAAGAAGCU | rA rA rA rG rC rU rG rG rA rC rA rA rG rA rA rG rC | OOOOOOOOOOOOOOOO | 87 |
| 1297 | AUU | rU rAmUmU | OOOO | |
| WV- | UAGCUUCUUGUCCAGCUU | PO fU rA rGmC rU rU rC rU rU rG rU rC rC rA rG | OOOOOOOOOOOOOOOO | 88 |
| 1298 | UUU | rC rU rU rUmUmU | OOOO | |
| WV- | UAGCUUCUUGUCCAGCUU | PO fUmA fGmC fUmU fCmU fUmG fUmC fCmA | OOOOOOOOOOOOOOOO | 89 |
| 1299 | UUU | fGmC fUmU fUmUmU | OOOO | |
| WV- | UAGCUUCUUGUCCAGCUU | PO fU * mA fG * mC fU * mU fC * mU fU * mG fU | XOXOXOXOXOXOXOXOXO | 90 |
| 1300 | UUU | * mC fC * mA fG * mC fU * mU fU * mUmU | XO | |
| WV- | UAGCUUCUUGUCCAGCUU | PO fU * mA * fGmC * fUmU * fCmU * fUmG * | XXOXOXOXOXOXOXOXOX | 91 |
| 1301 | UUU | fUmC * fCmA * fGmC * fUmU * fUmU * mU | OX | |
| WV- | UAGCUUCUUGUCCAGCUU | PO fU * mA * fG * mC * fU * mU * fC * mU * fU * | XXXXXXXXXXXXXXXXXXXX | 92 |
| 1302 | UUU | mG * fU * mC * fC * mA * fG * mC * fU * mU * | | |
| | | fU * mU * mU | | |
| WV- | UAGCUUCUUGUCCAGCUU | PO fU rA rG fC fU fU fC fU fU rG fU fC fC rA rG fC | OOOOOOOOOOOOOOOO | 93 |
| 1303 | UUU | fU fU fUmUmU | OOOO | |
| WV- | UAGCUUCUUGUCCAGCUU | PO fU * mA fGmC fUmU fCmU fUmG fUmC fCmA | XOOOOOOOOOOOOXXXX | 94 |
| 1304 | UUU | * fG * mC * fU * mU * fU * mU * mU | XXX | |
| WV- | UAGCUUCUUGUCCAGCUU | PO fUmA fGmC fUmU fCmU fUmG fUmC fCmA * | OOOOOOOOOOOOXXXX | 95 |
| 1305 | UUU | fG * mC * fU * mU * fU * mU * mU | XXX | |
| WV- | UAGCUUCUUGUCCAGCUU | PO fU * mA * fGmC fUmU fCmU fUmG fUmC | XXOOOOOOOOOOOXXXXX | 96 |
| 1306 | UUU | fCmA * fG * mC * fU * mU * fU * mU * mU | XX | |
| WV- | UAGCUUCUUGUCCAGCUU | PO fU * mA * fGmC * fUmU * fCmU * fUmG * | XXOXOXOXOXOXOXXXXXX | 97 |
| 1307 | UUU | fUmC * fCmA * fG * mC * fU * mU * fU * mU * | X | |
| | | mU | | |
| WV- | UAGCUUCUUGUCCAGCUU | POmU * fA * mG fC * mU fU * mC fU * mU fG * | XXOXOXOXOXOXOXXXXXX | 98 |
| 1308 | UUU | mU fC * mC fA * mG * fC * mU * fU * mU * | O | |
| | | mUmU | | |
| WV- | GCUUCAGAGGCCGAGGAU | rG rC rU rU rC rA rG rA rG rG rC rC rG rA rG rG rA | OOOOOOOOOOOOOOOO | 99 |
| 1309 | GUU | rU rGmUmU | OOOO | |
| WV- | CAUCCUCGGCCUCUGAAG | PO fCmA fUmC fCmU fCmG fGmC fCmU fCmU | OOOOOOOOOOOOOOOO | 100 |
| 1310 | CUU | fGmA fAmG fCmUmU | OOOO | |
| WV- | CAUCCUCGGCCUCUGAAG | PO fC * mA fU * mC fC * mU fC * mG fG * mC fC | XOXOXOXOXOXOXOXOXO | 101 |
| 1311 | CUU | * mU fC * mU fG * mA fA * mG fC * mUmU | XO | |
| WV- | CAUCCUCGGCCUCUGAAG | PO fC rA fU fC fC fU fC rG rG fC fC fU fC fU rG rA | OOOOOOOOOOOOOOOO | 102 |
| 1312 | CUU | rA rG fCmUmU | OOOO | |
| WV- | CAUCCUCGGCCUCUGAAG | PO fCmA fUmC fCmU fCmG fGmC fCmU fCmU * | OOOOOOOOOOOOOXXXX | 103 |
| 1313 | CUU | fG * mA * fA * mG * fC * mU * mU | XXX | |
| WV- | AUGAAGCACGCCACCAAG | rA rU rG rA rA rG rC rA rC rG rC rC rA rC rC rA rA | OOOOOOOOOOOOOOOO | 104 |
| 1314 | AUU | rG rAmUmU | OOOO | |
| WV- | UCUUGGUGGCGUGCUUC | PO fUmC fUmU fGmG fUmG fGmC fGmU fGmC | OOOOOOOOOOOOOOOO | 105 |
| 1315 | AUUU | fUmU fCmA fUmUmU | OOOO | |
| WV- | UCUUGGUGGCGUGCUUC | PO fU * mC fU * mU fG * mG fU * mG fG * mC fG | XOXOXOXOXOXOXOXOXO | 106 |
| 1316 | AUUU | * mU fG * mC fU * mU fC * mA fU * mUmU | XO | |
| WV- | UCUUGGUGGCGUGCUUC | PO fU fC fU fU rG rG fU rG rG fC rG fU rG fC fU fU | OOOOOOOOOOOOOOOO | 107 |
| 1317 | AUUU | fC rA fUmUmU | OOOO | |
| WV- | UCUUGGUGGCGUGCUUC | PO fUmC fUmU fGmG fUmG fGmC fGmU fGmC * | OOOOOOOOOOOOOXXXX | 108 |
| 1318 | AUUU | fU * mU * fC * mA * fU * mU * mU | XXX | |
| WV- | UGAGGUCAGACCAACUUC | rU rG rA rG rG rU rC rA rG rA rC rC rA rA rC rU rU | OOOOOOOOOOOOOOOO | 109 |
| 1319 | AUU | rC rAmUmU | OOOO | |
| WV- | UGAAGUUGGUCUGACCUC | PO rU rG rA rA rG rU rU rG rG rU rC rU rG rA rC | OOOOOOOOOOOOOOOO | 110 |
| 1320 | AUU | rC rU rC rAmUmU | OOOO | |
| WV- | UGAAGUUGGUCUGACCUC | PO fUmG fAmA fGmU fUmG fGmU fCmU fGmA | OOOOOOOOOOOOOOOO | 111 |
| 1321 | AUU | fCmC fUmC fAmUmU | OOOO | |
| WV- | UGAAGUUGGUCUGACCUC | PO fU * mG fA * mA fG * mU fU * mG fG * mU fC | XOXOXOXOXOXOXOXOXO | 112 |
| 1322 | AUU | * mU fG * mA fC * mC fU * mC fA * mUmU | XO | |
| WV- | UGAAGUUGGUCUGACCUC | PO fU * mG * fA * mA * fG * mU * fU * mG * fG | XXXXXXXXXXXXXXXXXXXX | 113 |
| 1323 | AUU | * mU * fC * mU * fG * mA * fC * mC * fU * mC * | | |
| | | fA * mU * mU | | |
| WV- | UGAAGUUGGUCUGACCUC | PO fU rG rA rA rG fU fU rG rG fU fC fU rG rA fC fC | OOOOOOOOOOOOOOOO | 114 |
| 1324 | AUU | fU fC rAmUmU | OOOO | |
| WV- | UGAAGUUGGUCUGACCUC | PO fUmG fAmA fGmU fUmG fGmU fCmU fGmA * | OOOOOOOOOOOOOXXXX | 115 |
| 1325 | AUU | fC * mC * fU * mC * fA * mU * mU | XXX | |
| WV- | AGGGUUACAUGAAGCACG | rA rG rG rG rU rU rA rC rA rU rG rA rA rG rC rA rC | OOOOOOOOOOOOOOOO | 116 |
| 1326 | CUU | rG rCmUmU | OOOO | |
| WV- | GCGUGCUUCAUGUAACCC | PO fGmC fGmU fGmC fUmU fCmA fUmG fUmA | OOOOOOOOOOOOOOOO | 117 |
| 1327 | UUU | fAmC fCmC fUmUmU | OOOO | |
| WV- | GCGUGCUUCAUGUAACCC | PO fG * mC fG * mU fG * mC fU * mU fC * mA fU | XOXOXOXOXOXOXOXOXO | 118 |
| 1328 | UUU | * mG fU * mA fA * mC fC * mC fU * mUmU | XO | |
| WV- | GCGUGCUUCAUGUAACCC | PO rG fC rG fU rG fC fU fU fC rA fU rG fU rA rA fC | OOOOOOOOOOOOOOOO | 119 |
| 1329 | UUU | fC fC fUmUmU | OOOO | |
| WV- | GCGUGCUUCAUGUAACCC | PO fGmC fGmU fGmC fUmU fCmA fUmG fUmA * | OOOOOOOOOOOOOXXXX | 120 |
| 1330 | UUU | fA * mC * fC * mC * fU * mU * mU | XXX | |
| WV- | AAUACUGUCCCUUUUAAG | rA * R rA rU rA rC rU rG rU rC rC rC rU rU rU rU | ROOOOOOOOOOOOOOO | 121 |
| 1331 | CUU | rA rA rG rCmUmU | OOOO | |
| WV- | AAUACUGUCCCUUUUAAG | rA rA * R rU rA rC rU rG rU rC rC rC rU rU rU rU | OROOOOOOOOOOOOOO | 122 |
| 1332 | CUU | rA rA rG rCmUmU | OOOO | |
| WV- | AAUACUGUCCCUUUUAAG | rA rA rU * R rA rC rU rG rU rC rC rC rU rU rU rU | OOROOOOOOOOOOOOO | 123 |
| 1333 | CUU | rA rA rG rCmUmU | OOOO | |
| WV- | AAUACUGUCCCUUUUAAG | rA rA rU rA * R rC rU rG rU rC rC rC rU rU rU rU | OOOROOOOOOOOOOOO | 124 |
| 1334 | CUU | rA rA rG rCmUmU | OOOO | |
| WV- | AAUACUGUCCCUUUUAAG | rA rA rU rA rC * R rU rG rU rC rC rC rU rU rU rU | OOOOROOOOOOOOOOO | 125 |
| 1335 | CUU | rA rA rG rCmUmU | OOOO | |
| WV- | AAUACUGUCCCUUUUAAG | rA rA rU rA rC rU * R rG rU rC rC rC rU rU rU rU | OOOOOROOOOOOOOOO | 126 |
| 1336 | CUU | rA rA rG rCmUmU | OOOO | |
| WV- | AAUACUGUCCCUUUUAAG | rA rA rU rA rC rU rG * R rU rC rC rC rU rU rU rU | OOOOOOROOOOOOOOO | 127 |
| 1337 | CUU | rA rA rG rCmUmU | OOOO | |
| WV- | AAUACUGUCCCUUUUAAG | rA rA rU rA rC rU rG rU * R rC rC rC rU rU rU rU | OOOOOOOROOOOOOOO | 128 |
| 1338 | CUU | rA rA rG rCmUmU | OOOO | |
| WV- | AAUACUGUCCCUUUUAAG | rA rA rU rA rC rU rG rU rC * R rC rC rU rU rU rU | OOOOOOOOROOOOOOO | 129 |
| 1339 | CUU | rA rA rG rCmUmU | OOOO | |
| WV- | AAUACUGUCCCUUUUAAG | rA rA rU rA rC rU rG rU rC rC * R rC rU rU rU rU | OOOOOOOOOROOOOOO | 130 |
| 1340 | CUU | rA rA rG rCmUmU | OOOO | |
| WV- | AAUACUGUCCCUUUUAAG | rA rA rU rA rC rU rG rU rC rC rC * R rU rU rU rU | OOOOOOOOOOROOOOO | 131 |
| 1341 | CUU | rA rA rG rCmUmU | OOOO | |
| WV- | AAUACUGUCCCUUUUAAG | rA rA rU rA rC rU rG rU rC rC rC rU * R rU rU rU | OOOOOOOOOOOROOOO | 132 |
| 1342 | CUU | rA rA rG rCmUmU | OOOO | |
| WV- | AAUACUGUCCCUUUUAAG | rA rA rU rA rC rU rG rU rC rC rC rU rU * R rU rU | OOOOOOOOOOOOROOO | 133 |
| 1343 | CUU | rA rA rG rCmUmU | OOOO | |
| WV- | AAUACUGUCCCUUUUAAG | rA rA rU rA rC rU rG rU rC rC rC rU rU rU * R rU | OOOOOOOOOOOOOROO | 134 |
| 1344 | CUU | rA rA rG rCmUmU | OOOO | |
| WV- | AAUACUGUCCCUUUUAAG | rA rA rU rA rC rU rG rU rC rC rC rU rU rU rU * R | OOOOOOOOOOOOOORO | 135 |
| 1345 | CUU | rA rA rG rCmUmU | OOOO | |
| WV- | AAUACUGUCCCUUUUAAG | rA rA rU rA rC rU rG rU rC rC rC rU rU rU rU rA * | OOOOOOOOOOOOOOOR | 136 |
| 1346 | CUU | R rA rG rCmUmU | OOOO | |
| WV- | AAUACUGUCCCUUUUAAG | rA rA rU rA rC rU rG rU rC rC rC rU rU rU rU rA rA | OOOOOOOOOOOOOOOO | 137 |
| 1347 | CUU | * R rG rCmUmU | ROOO | |
| WV- | AAUACUGUCCCUUUUAAG | rA rA rU rA rC rU rG rU rC rC rC rU rU rU rU rA rA | OOOOOOOOOOOOOOOO | 138 |
| 1348 | CUU | rG * R rCmUmU | OROO | |
| WV- | AAUACUGUCCCUUUUAAG | rA rA rU rA rC rU rG rU rC rC rC rU rU rU rU rA rA | OOOOOOOOOOOOOOOO | 139 |
| 1349 | CUU | rG rC * RmUmU | OORO | |
| WV- | AAUACUGUCCCUUUUAAG | rA rA rU rA rC rU rG rU rC rC rC rU rU rU rU rA rA | OOOOOOOOOOOOOOOO | 140 |
| 1350 | CUU | rG rCmU * RmU | OOOR | |
| WV- | AAUACUGUCCCUUUUAAG | rA * S rA rU rA rC rU rG rU rC rC rC rU rU rU rU | SOOOOOOOOOOOOOOO | 141 |
| 1351 | CUU | rA rA rG rCmUmU | OOOO | |
| WV- | AAUACUGUCCCUUUUAAG | rA rA * S rU rA rC rU rG rU rC rC rC rU rU rU rU | OSOOOOOOOOOOOOOO | 142 |
| 1352 | CUU | rA rA rG rCmUmU | OOOO | |
| WV- | AAUACUGUCCCUUUUAAG | rA rA rU * S rA rC rU rG rU rC rC rC rU rU rU rU | OOSOOOOOOOOOOOOO | 143 |
| 1353 | CUU | rA rA rG rCmUmU | OOOO | |
| WV- | AAUACUGUCCCUUUUAAG | rA rA rU rA * S rC rU rG rU rC rC rC rU rU rU rU | OOOSOOOOOOOOOOOO | 144 |
| 1354 | CUU | rA rA rG rCmUmU | OOOO | |
| WV- | AAUACUGUCCCUUUUAAG | rA rA rU rA rC * S rU rG rU rC rC rC rU rU rU rU | OOOOSOOOOOOOOOOO | 145 |
| 1355 | CUU | rA rA rG rCmUmU | OOOO | |
| WV- | AAUACUGUCCCUUUUAAG | rA rA rU rA rC rU * S rG rU rC rC rC rU rU rU rU | OOOOOSOOOOOOOOOO | 146 |
| 1356 | CUU | rA rA rG rCmUmU | OOOO | |
| WV- | AAUACUGUCCCUUUUAAG | rA rA rU rA rC rU rG * S rU rC rC rC rU rU rU rU | OOOOOOSOOOOOOOOO | 147 |
| 1357 | CUU | rA rA rG rCmUmU | OOOO | |
| WV- | AAUACUGUCCCUUUUAAG | rA rA rU rA rC rU rG rU * S rC rC rC rU rU rU rU | OOOOOOOSOOOOOOOO | 148 |
| 1358 | CUU | rA rA rG rCmUmU | OOOO | |
| WV- | AAUACUGUCCCUUUUAAG | rA rA rU rA rC rU rG rU rC * S rC rC rU rU rU rU | OOOOOOOOSOOOOOOO | 149 |
| 1359 | CUU | rA rA rG rCmUmU | OOOO | |
| WV- | AAUACUGUCCCUUUUAAG | rA rA rU rA rC rU rG rU rC rC * S rC rU rU rU rU | OOOOOOOOOSOOOOOO | 150 |
| 1360 | CUU | rA rA rG rCmUmU | OOOO | |
| WV- | AAUACUGUCCCUUUUAAG | rA rA rU rA rC rU rG rU rC rC rC * S rU rU rU rU | OOOOOOOOOOSOOOOO | 151 |
| 1361 | CUU | rA rA rG rCmUmU | OOOO | |
| WV- | AAUACUGUCCCUUUUAAG | rA rA rU rA rC rU rG rU rC rC rC rU * S rU rU rU | OOOOOOOOOOOSOOOO | 152 |
| 1362 | CUU | rA rA rG rCmUmU | OOOO | |
| WV- | AAUACUGUCCCUUUUAAG | rA rA rU rA rC rU rG rU rC rC rC rU rU * S rU rU | OOOOOOOOOOOOSOOO | 153 |
| 1363 | CUU | rA rA rG rCmUmU | OOOO | |
| WV- | AAUACUGUCCCUUUUAAG | rA rA rU rA rC rU rG rU rC rC rC rU rU rU * S rU | OOOOOOOOOOOOOSOO | 154 |
| 1364 | CUU | rA rA rG rCmUmU | OOOO | |
| WV- | AAUACUGUCCCUUUUAAG | rA rA rU rA rC rU rG rU rC rC rC rU rU rU rU * S | OOOOOOOOOOOOOOSO | 155 |
| 1365 | CUU | rA rA rG rCmUmU | OOOO | |
| WV- | AAUACUGUCCCUUUUAAG | rA rA rU rA rC rU rG rU rC rC rC rU rU rU rU rA * | OOOOOOOOOOOOOOOS | 156 |
| 1366 | CUU | S rA rG rCmUmU | OOOO | |
| WV- | AAUACUGUCCCUUUUAAG | rA rA rU rA rC rU rG rU rC rC rC rU rU rU rU rA rA | OOOOOOOOOOOOOOOO | 157 |
| 1367 | CUU | * S rG rCmUmU | SOOO | |
| WV- | AAUACUGUCCCUUUUAAG | rA rA rU rA rC rU rG rU rC rC rC rU rU rU rU rA rA | OOOOOOOOOOOOOOOO | 158 |
| 1368 | CUU | rG * S rCmUmU | OSOO | |
| WV- | AAUACUGUCCCUUUUAAG | rA rA rU rA rC rU rG rU rC rC rC rU rU rU rU rA rA | OOOOOOOOOOOOOOOO | 159 |
| 1369 | CUU | rG rC * SmUmU | OOSO | |
| WV- | AAUACUGUCCCUUUUAAG | rA rA rU rA rC rU rG rU rC rC rC rU rU rU rU rA rA | OOOOOOOOOOOOOOOO | 160 |
| 1370 | CUU | rG rCmU * SmU | OOOS | |
| WV- | UCCAGCUUUAUUGGGAG | PO rU rC rC rA rG rC rU rU rU rA rU rU rG rG rG | OOOOOOOOOOOOOOOO | 161 |
| 1498 | GCUU | rA rG rG rCmUmU | OOOO | |
| WV- | AGGUCUCAGGCAGCCACG | PO rA rG rG rU rC rU rC rA rG rG rC rA rG rC rC rA | OOOOOOOOOOOOOOOO | 162 |
| 1499 | GUU | rC rG rGmUmU | OOOO | |
| WV- | AGGCCAGCAUGCCUGGAG | PO rA rG rG rC rC rA rG rC rA rU rG rC rC rU rG rG | OOOOOOOOOOOOOOOO | 163 |
| 1500 | GUU | rA rG rGmUmU | OOOO | |
| WV- | CCACCUGGGACUCCUGCA | PO rC rC rA rC rC rU rG rG rG rA rC rU rC rC rU rG | OOOOOOOOOOOOOOOO | 164 |
| 1501 | CUU | rC rA rCmUmU | OOOO | |
| WV- | GCCAUCGGUCACCCAGCCC | PO rG rC rC rA rU rC rG rG rU rC rA rC rC rC rA rG | OOOOOOOOOOOOOOOO | 165 |
| 1502 | UU | rC rC rCmUmU | OOOO | |
| WV- | GUGCAUCCUUGGCGGUCU | PO rG rU rG rC rA rU rC rC rU rU rG rG rC rG rG | OOOOOOOOOOOOOOOO | 166 |
| 1503 | UUU | rU rC rU rUmUmU | OOOO | |
| WV- | CUGCUGGGCCACCUGGGA | PO rC rU rG rC rU rG rG rG rC rC rA rC rC rU rG rG | OOOOOOOOOOOOOOOO | 167 |
| 1504 | CUU | rG rA rCmUmU | OOOO | |
| WV- | CAUCCUCGGCCUCUGAAG | PO rC rA rU rC rC rU rC rG rG rC rC rU rC rU rG rA | OOOOOOOOOOOOOOOO | 168 |
| 1505 | CUU | rA rG rCmUmU | OOOO | |
| WV- | UCUUGGUGGCGUGCUUC | PO rU rC rU rU rG rG rU rG rG rC rG rU rG rC rU | OOOOOOOOOOOOOOOO | 169 |
| 1506 | AUUU | rU rC rA rUmUmU | OOOO | |
| WV- | GCGUGCUUCAUGUAACCC | PO rG rC rG rU rG rC rU rU rC rA rU rG rU rA rA | OOOOOOOOOOOOOOOO | 170 |
| 1507 | UUU | rC rC rC rUmUmU | OOOO | |
| WV- | AAUACUGUCCCUUUUAAG | rA * rA * rU * rA * rC * rU * rG * rU * rC * rC * | XXXXXXXXXXXXXXXXXXXX | 171 |
| 1516 | CUU | rC * rU * rU * rU * rU * rA * rA * rG * rC * mU * | | |
| | | mU | | |
| WV- | GCUUAAAAGGGACAGUAU | rG rC rU rU rA rA rA rA rG rG rG rA rC rA rG rU rA | OOOOOOOOOOOOOOOO | 172 |
| 1652 | U | rU rU | OO | |
| WV- | GUUGCUUAAAAGGG | rG rU rU rG rC rU rU rA rA rA rA rG rG rG rA rC | OOOOOOOOOOO | 173 |
| 1653 | ACAGUAUUCUC | rA rG rU rA rU rU rC rU rC | OOOOOOOOOOOOO | |
| WV- | TCACTGAGAATACTGTCCC | VPTeo * fC * mA fC * mT fG * mA fG * mA fA * | XXOXOXOXOXOXOXXXXXX | 174 |
| 1783 | AA | mT fA * mC fT * mG * fT * mC * fC * mC * Aeo * | X | |
| | | Aeo | | |
| WV- | UCACUGAGAAUACUGUCC | VPUeo * fC * mA fC * mU fG * mA fG * mA fA * | XXOXOXOXOXOXOXXXXXX | 175 |
| 1784 | CAA | mU fA * mC fU * mG * fU * mC * fC * mC * Aeo * | X | |
| | | Aeo | | |
| WV- | UAGCUUCUUGUCCAGCUU | PO fU * fA * fGmC * fUmU * fCmU * fUmG * | XXOXOXOXOXOXOXXXXXX | 176 |
| 1791 | UUU | fUmC * fCmA * fG * mC * fU * mU * fU * mUmU | O | |
| WV- | UAGCUUCUUGUCCAGCUU | PO fU * fA * mGmC * fUmU * fCmU * fUmG * | XXOXOXOXOXOXOXXXXXX | 177 |
| 1792 | UUU | fUmC * fCmA * fG * mC * fU * mU * fU * mUmU | O | |
| WV- | UAGCUUCUUGUCCAGCUU | PO fU * mA * fGmC * fUmU * fCmU * fUmG * | XXOXOXOXOXOXOXXXXXX | 178 |
| 1793 | UUU | fUmC * fCmA * fG * mC * fU * mU * fU * mUmU | O | |
| WV- | UAGCUUCUUGUCCAGCUU | PO fU * fA * fG fC * mU fU * mC fU * mU fG * | XXOXOXOXOXOXOXXXXXX | 179 |
| 1794 | UUU | mU fC * mC fA * mG * fC * mU * fU * mU * | O | |
| | | mUmU | | |
| WV- | UAGCUUCUUGUCCAGCUU | PO fU * fA * mG fC * mU fU * mC fU * mU fG * | XXOXOXOXOXOXOXXXXXX | 180 |
| 1795 | UUU | mU fC * mC fA * mG * fC * mU * fU * mU * | O | |
| | | mUmU | | |
| WV- | UAGCUUCUUGUCCAGCUU | POmU * fA * fG fC * mU fU * mC fU * mU fG * | XXOXOXOXOXOXOXXXXXX | 181 |
| 1796 | UUU | mU fC * mC fA * mG * fC * mU * fU * mU * | O | |
| | | mUmU | | |
| WV- | UCACUGAGAAUACUGUCC | PO fU * fC * fA fC * mU fG * mA fG * mA fA * mU | XXOXOXOXOXOXOXXXXXX | 182 |
| 1797 | CUU | fA * mC fU * mG * fU * mC * fC * mC * mUmU | O | |
| WV- | UCACUGAGAAUACUGUCC | PO fU * fC * mA fC * mU fG * mA fG * mA fA * | XXOXOXOXOXOXOXXXXXX | 183 |
| 1798 | CUU | mU fA * mC fU * mG * fU * mC * fC * mC * | O | |
| | | mUmU | | |
| WV- | GUGCAUCCUUGGCGGUCU | POmG * fU * mG fC * mA fU * mC fC * mU fU * | XXOXOXOXOXOXOXXXXXX | 184 |
| 1800 | UUU | mG fG * mC fG * mG * fU * mC * fU * mU * | O | |
| | | mUmU | | |
| WV- | GUGCAUCCUUGGCGGUCU | PO fG * fU * fGmC * mAmU * mCmC * mU fU | XXOXOXOXOOOXOOXXXX | 185 |
| 1801 | UUU | fGmG * mC fGmG * fU * mC * fU * mU * mUmU | XO | |
| WV- | GUGCAUCCUUGGCGGUCU | PO fG * fU * fG fC * mA fU * mC fC * mU fU fG fG | XXOXOXOXOOOXOOXXXX | 186 |
| 1802 | UUU | * mC fGmG * fU * mC * fU * mU * mUmU | XO | |
| WV- | GUGCAUCCUUGGCGGUCU | POmG * fU * mG fC * mA fU * mC fC * mU fU fG | XXOXOXOXOOOXOOXXXX | 187 |
| 1803 | UUU | fG * mC fGmG * fU * mC * fU * mU * mUmU | XO | |
| WV- | CUGCUGGGCCACCUGGGA | POmC * fU * mG fC * mU fG * mG fG * mC fC * | XXOXOXOXOXOXOXXXXXX | 188 |
| 1804 | CUU | mA fC * mC fU * mG * fG * mG * fA * mC * | O | |
| | | mUmU | | |
| WV- | CUGCUGGGCCACCUGGGA | PO fC * fU * fGmC * mUmG * mGmG * mC fC | XXOXOXOXOOOXOOXXXX | 189 |
| 1805 | CUU | fAmC * mC fUmG * fG * mG * fA * mC * mUmU | XO | |
| WV- | CUGCUGGGCCACCUGGGA | PO fC * fU * fG fC * mU fG * mG fG * mC fC fA fC | XXOXOXOXOOOXOOXXXX | 190 |
| 1806 | CUU | * mC fUmG * fG * mG * fA * mC * mUmU | XO | |
| WV- | CUGCUGGGCCACCUGGGA | POmC * fU * mG fC * mU fG * mG fG * mC fC fA | XXOXOXOXOOOXOOXXXX | 191 |
| 1807 | CUU | fC * mC fUmG * fG * mG * fA * mC * mUmU | XO | |
| WV- | UUGGUCUGACCUCAGGGU | POmU * fU * mG fG * mU fC * mU fG * mA fC * | XXOXOXOXOXOXOXXXXXX | 192 |
| 1808 | CUU | mC fU * mC fA * mG * fG * mG * fU * mC * | O | |
| | | mUmU | | |
| WV- | UUGGUCUGACCUCAGGGU | PO fU * fU * fGmG * mUmC * mUmG * mA fC | XXOXOXOXOOOXOOXXXX | 193 |
| 1809 | CUU | fCmU * mC fAmG * fG * mG * fU * mC * mUmU | XO | |
| WV- | UUGGUCUGACCUCAGGGU | PO fU * fU * fG fG * mU fC * mU fG * mA fC fC fU | XXOXOXOXOOOXOOXXXX | 194 |
| 1810 | CUU | * mC fAmG * fG * mG * fU * mC * mUmU | XO | |
| WV- | UUGGUCUGACCUCAGGGU | POmU * fU * mG fG * mU fC * mU fG * mA fC fC | XXOXOXOXOOOXOOXXXX | 195 |
| 1811 | CUU | fU * mC fAmG * fG * mG * fU * mC * mUmU | XO | |
| WV- | AGGCCAGCAUGCCUGGAG | POmA * fG * mG fC * mC fA * mG fC * mA fU * | XXOXOXOXOXOXOXXXXXX | 196 |
| 1812 | GUU | mG fC * mC fU * mG * fG * mA * fG * mG * | O | |
| | | mUmU | | |
| WV- | AGGCCAGCAUGCCUGGAG | PO fA * fG * fGmC * mCmA * mGmC * mA fU | XXOXOXOXOOOXOOXXXX | 197 |
| 1813 | GUU | fGmC * mC fUmG * fG * mA * fG * mG * mUmU | XO | |
| WV- | AGGCCAGCAUGCCUGGAG | PO fA * fG * fG fC * mC fA * mG fC * mA fU fG fC | XXOXOXOXOOOXOOXXXX | 198 |
| 1814 | GUU | * mC fUmG * fG * mA * fG * mG * mUmU | XO | |
| WV- | AGGCCAGCAUGCCUGGAG | POmA * fG * mG fC * mC fA * mG fC * mA fU fG | XXOXOXOXOOOXOOXXXX | 199 |
| 1815 | GUU | fC * mC fUmG * fG * mA * fG * mG * mUmU | XO | |
| WV- | CCACCUGGGACUCCUGCA | POmC * fC * mA fC * mC fU * mG fG * mG fA * | XXOXOXOXOXOXOXXXXXX | 200 |
| 1816 | CUU | mC fU * mC fC * mU * fG * mC * fA * mC * | O | |
| | | mUmU | | |
| WV- | CCACCUGGGACUCCUGCA | PO fC * fC * fAmC * mCmU * mGmG * mG fA | XXOXOXOXOOOXOOXXXX | 201 |
| 1817 | CUU | fCmU * mC fCmU * fG * mC * fA * mC * mUmU | XO | |
| WV- | CCACCUGGGACUCCUGCA | PO fC * fC * fA fC * mC fU * mG fG * mG fA fC fU | XXOXOXOXOOOXOOXXXX | 202 |
| 1818 | CUU | * mC fCmU * fG * mC * fA * mC * mUmU | XO | |
| WV- | CCACCUGGGACUCCUGCA | POmC * fC * mA fC * mC fU * mG fG * mG fA fC | XXOXOXOXOOOXOOXXXX | 203 |
| 1819 | CUU | fU * mC fCmU * fG * mC * fA * mC * mUmU | XO | |
| WV- | GCCAUCGGUCACCCAGCCC | POmG * fC * mC fA * mU fC * mG fG * mU fC * | XXOXOXOXOXOXOXXXXXX | 204 |
| 1820 | UU | mA fC * mC fC * mA * fG * mC * fC * mC * | O | |
| | | mUmU | | |
| WV- | GCCAUCGGUCACCCAGCCC | PO fG * fC * fCmA * mUmC * mGmG * mU fC | XXOXOXOXOOOXOOXXXX | 205 |
| 1821 | UU | fAmC * mC fCmA * fG * mC * fC * mC * mUmU | XO | |
| WV- | GCCAUCGGUCACCCAGCCC | PO fG * fC * fC fA * mU fC * mG fG * mU fC fA fC | XXOXOXOXOOOXOOXXXX | 206 |
| 1822 | UU | * mC fCmA * fG * mC * fC * mC * mUmU | XO | |
| WV- | GCCAUCGGUCACCCAGCCC | POmG * fC * mC fA * mU fC * mG fG * mU fC fA | XXOXOXOXOOOXOOXXXX | 207 |
| 1823 | UU | fC * mC fCmA * fG * mC * fC * mC * mUmU | XO | |
| WV- | UCCAGCUUUAUUGGGAG | POmU * fC * mC fA * mG fC * mU fU * mU fA * | XXOXOXOXOXOXOXXXXXX | 208 |
| 1824 | GCUU | mU fU * mG fG * mG * fA * mG * fG * mC * | O | |
| | | mUmU | | |
| WV- | UCCAGCUUUAUUGGGAG | PO fU * fC * fCmA * mGmC * mUmU * mU fA | XXOXOXOXOOOXOOXXXX | 209 |
| 1825 | GCUU | fUmU * mG fGmG * fA * mG * fG * mC * mUmU | XO | |
| WV- | UCCAGCUUUAUUGGGAG | PO fU * fC * fC fA * mG fC * mU fU * mU fA fU fU | XXOXOXOXOOOXOOXXXX | 210 |
| 1826 | GCUU | * mG fGmG * fA * mG * fG * mC * mUmU | XO | |
| WV- | UCCAGCUUUAUUGGGAG | POmU * fC * mC fA * mG fC * mU fU * mU fA fU | XXOXOXOXOOOXOOXXXX | 211 |
| 1827 | GCUU | fU * mG fGmG * fA * mG * fG * mC * mUmU | XO | |
| WV- | AGGUCUCAGGCAGCCACG | POmA * fG * mG fU * mC fU * mC fA * mG fG * | XXOXOXOXOXOXOXXXXXX | 212 |
| 1828 | GUU | mC fA * mG fC * mC * fA * mC * fG * mG * | O | |
| | | mUmU | | |
| WV- | AGGUCUCAGGCAGCCACG | PO fA * fG * fGmU * mCmU * mCmA * mG fG | XXOXOXOXOOOXOOXXXX | 213 |
| 1829 | GUU | fCmA * mG fCmC * fA * mC * fG * mG * mUmU | XO | |
| WV- | AGGUCUCAGGCAGCCACG | PO fA * fG * fG fU * mC fU * mC fA * mG fG fC fA | XXOXOXOXOOOXOOXXXX | 214 |
| 1830 | GUU | * mG fCmC * fA * mC * fG * mG * mUmU | XO | |
| WV- | AGGUCUCAGGCAGCCACG | POmA * fG * mG fU * mC fU * mC fA * mG fG fC | XXOXOXOXOOOXOOXXXX | 215 |
| 1831 | GUU | fA * mG fCmC * fA * mC * fG * mG * mUmU | XO | |
| WV- | UGUCCAGCUUUAUUGGG | POmU * fG * mU fC * mC fA * mG fC * mU fU * | XXOXOXOXOXOXOXXXXXX | 216 |
| 2110 | AGUU | mU fA * mU fU * mG * fG * mG * fA * mG * | O | |
| | | mUmU | | |
| WV- | UGUCCAGCUUUAUUGGG | POmU * fG * mU fC * mC fA * mG fC * mU fU * | XXOXOXOXOXO | 217 |
| 2111 | AGGCCAUU | mU fA * mU fU * G * G * G * A * G * G * C * C * | XOXXXXXXXXXXO | |
| | | A * mUmU | | |
| WV- | UAGCUUCUUGUCCAGCTT | POmU * fA * mG fC * mU fU * mC fU * mU fG * | XXOXOXOXOXO | 218 |
| 2114 | TATTGUU | mU fC * mC fA * G * C * T * T * T * A * T * T * G | XOXXXXXXXXXXO | |
| | | * mUmU | | |
| WV- | UAGCUUCUUGUCCAGCUU | POmU * fA * mG fC * mU fU * mC fU * mU fG * | XXOXOXOXOXOXOXXXXXX | 219 |
| 2150 | UTU | mU fC * mC fA * mG * fC * mU * fU * mU * T * | X | |
| | | mU | | |
| WV- | UAGCUUCUUGUCCAGCUU | POmU * fA * mG fC * mU fU * mC fU * mU fG * | XXOXOXOXOXOXOXXXXXX | 220 |
| 2151 | TUU | mU fC * mC fA * mG * fC * mU * fU * T * mUmU | O | |
| WV- | UAGCUUCUUGUCC | POmU * fA * mG fC * mU fU * mC fU * mU fG * | XXOXOXOXOXO | 221 |
| 2152 | AGCTTTATTGTU | mU fC * mC fA * G * C * T * T * T * A * T * T * G | XOXXXXXXXXXXX | |
| | | * T * mU | | |
| WV- | UAGCUUCUUGUCC | POmU * fA * mG fC * mU fU * mC fU * mU fG * | XXOXOXOXOXO | 222 |
| 2153 | AGCTTTATTTUU | mU fC * mCfA * G * C * T * T * T * A * T * T * T * | XOXXXXXXXXXXO | |
| | | mUmU | | |
| WV- | UGUCCAGCUUUAUUGGG | POmU * fG * mU fC * mC fA * mG fC * mU fU * | XXOXOXOXOXOXOXXXXXX | 223 |
| 2154 | AGTU | mU fA * mU fU * mG * fG * mG * fA * mG * T * | X | |
| | | mU | | |
| WV- | UGUCCAGCUUUAU | POmU * fG * mU fC * mC fA * mG fC * mU fU * | XXOXOXOXOXOXOXXXXXX | 224 |
| 2155 | UGGGATUU | mU fA * mU fU * mG * fG * mG * fA * T * mUmU | O | |
| WV- | UGUCCAGCUUUAU | POmU * fG * mU fC * mC fA * mG fC * mU fU * | XXOXOXOXOXO | 225 |
| 2156 | UGGGAGGCCATU | mU fA * mU fU * G * G * G * A * G * G * C * C * | XOXXXXXXXXXXX | |
| | | A * T * mU | | |
| WV- | UGUCCAGCUUUAU | POmU * fG * mU fC * mC fA * mG fC * mU fU * | XXOXOXOXOXO | 226 |
| 2157 | UGGGAGGCCTUU | mU fA * mU fU * G * G * G * A * G * G * C * C * T | XOXXXXXXXXXXO | |
| | | * mUmU | | |
| WV- | CAUAGCAGCUUCUUGUCC | POmC * fA * mU fA * mG fC * mA fG * mC fU * | XXOXOXOXOXOXOXXXXXX | 227 |
| 2166 | AUU | mU fC * mU fU * mG * fU * mC * fC * mA * | O | |
| | | mUmU | | |
| WV- | AUAGCAGCUUCUUGUCCA | POmA * fU * mA fG * mC fA * mG fC * mU fU * | XXOXOXOXOXOXOXXXXXX | 228 |
| 2167 | GUU | mC fU * mU fG * mU * fC * mC * fA * mG * | O | |
| | | mUmU | | |
| WV- | UAGCAGCUUCUUGUCCAG | POmU * fA * mG fC * mA fG * mC fU * mU fC * | XXOXOXOXOXOXOXXXXXX | 229 |
| 2168 | CUU | mU fU * mG fU * mC * fC * mA * fG * mC * | O | |
| | | mUmU | | |
| WV- | AGCAGCUUCUUGUCCAGC | POmA * fG * mC fA * mG fC * mU fU * mC fU * | XXOXOXOXOXOXOXXXXXX | 230 |
| 2169 | UUU | mU fG * mU fC * mC * fA * mG * fC * mU * | O | |
| | | mUmU | | |
| WV- | GCAGCUUCUUGUCCAGCU | POmG * fC * mA fG * mC fU * mU fC * mU fU * | XXOXOXOXOXOXOXXXXXX | 231 |
| 2170 | UUU | mG fU * mC fC * mA * fG * mC * fU * mU * | O | |
| | | mUmU | | |
| WV- | CAGCUUCUUGUCCAGCUU | POmC * fA * mG fC * mU fU * mC fU * mU fG * | XXOXOXOXOXOXOXXXXXX | 232 |
| 2171 | UUU | mU fC * mC fA * mG * fC * mU * fU * mU * | O | |
| | | mUmU | | |
| WV- | AGCUUCUUGUCCAGCUUU | POmA * fG * mC fU * mU fC * mU fU * mG fU * | XXOXOXOXOXOXOXXXXXX | 233 |
| 2172 | AUU | mC fC * mA fG * mC * fU * mU * fU * mA * | O | |
| | | mUmU | | |
| WV- | GCUUCUUGUCCAGCUUUA | POmG * fC * mU fU * mC fU * mU fG * mU fC * | XXOXOXOXOXOXOXXXXXX | 234 |
| 2173 | UUU | mC fA * mG fC * mU * fU * mU * fA * mU * | O | |
| | | mUmU | | |
| WV- | CUUCUUGUCCAGCUUUAU | POmC * fU * mU fC * mU fU * mG fU * mC fC * | XXOXOXOXOXOXOXXXXXX | 235 |
| 2174 | UUU | mA fG * mCfU * mU * fU * mA * fU * mU * | O | |
| | | mUmU | | |
| WV- | UUCUUGUCCAGCUUUAU | POmU * fU * mC fU * mU fG * mU fC * mC fA * | XXOXOXOXOXOXOXXXXXX | 236 |
| 2175 | UGUU | mG fC * mU fU * mU * fA * mU * fU * mG * | O | |
| | | mUmU | | |
| WV- | UCUUGUCCAGCUUUAUU | POmU * fC * mU fU * mG fU * mC fC * mA fG * | XXOXOXOXOXOXOXXXXXX | 237 |
| 2176 | GGUU | mC fU * mU fU * mA * fU * mU * fG * mG * | O | |
| | | mUmU | | |
| WV- | CUUGUCCAGCUUUAUUG | POmC * fU * mU fG * mU fC * mC fA * mG fC * | XXOXOXOXOXOXOXXXXXX | 238 |
| 2177 | GGUU | mU fU * mU fA * mU * fU * mG * fG * mG * | O | |
| | | mUmU | | |
| WV- | UUGUCCAGCUUUAUUGG | POmU * fU * mG fU * mC fC * mA fG * mC fU * | XXOXOXOXOXOXOXXXXXX | 239 |
| 2178 | GAUU | mU fU * mA fU * mU * fG * mG * fG * mA * | O | |
| | | mUmU | | |
| WV- | CAAUAAAGCUGG | rC rA rA rU rA rA rA rG rC rU rG rG rA rC rA rA rG | OOOOOOOOOO | 240 |
| 2372 | ACAAGAAGCUA | rA rA rG rC rU rA | OOOOOOOOOOOO | |
| WV- | UGGCCUCCCAAUAAAGCU | rU rG rG rC rC rU rC rC rC rA rA rU rA rA rA rG rC | OOOOOOOOOO | 241 |
| 2373 | GGACA | rU rG rG rA rC rA | OOOOOOOOOOOO | |
| WV- | UAGCUUCUUGUC | mU * fA * mG fC * mU fU * mC fU * mU fG * mU | XXOXOXOXOXOXOXXXXXX | 242 |
| 2386 | CAGCUUUUU | fC * mC fA * mG * fC * mU * fU * mU * mUmU | O | |
| WV- | UAGCUUCUUGUC | mU * fA * mG fC * mU fU * mC fU * mU fG * mU | XXOXOXOXOXO | 243 |
| 2387 | CAGCTTTATTGUU | fC * mC fA * G * C * T * T * T * A * T * T * G * | XOXXXXXXXXXXO | |
| | | mUmU | | |
| WV- | UGUCCAGCUUUA | mU * fG * mU fC * mC fA * mG fC * mU fU * mU | XXOXOXOXOXOXOXXXXXX | 244 |
| 2388 | UUGGGAGUU | fA * mU fU * mG * fG * mG * fA * mG * mUmU | O | |
| WV- | UGUCCAGCUUUA | mU * fG * mU fC * mC fA * mG fC * mU fU * mU | XXOXOXOXOXO | 245 |
| 2389 | UUGGGAGGCCAUU | fA * mU fU * G * G * G * A * G * G * C * C * A * | XOXXXXXXXXXXO | |
| | | mUmU | | |
| WV- | TAGCUUCUUGUCCAGCUU | POT * fA * mG fC * mU fU * mC fU * mU fG * mU | XXOXOXOXOXOXOXXXXXX | 246 |
| 2420 | UUU | fC * mC fA * mG * fC * mU * fU * mU * mUmU | O | |
| WV- | TAGCUUCUUGUCCAGCUU | T * fA * mG fC * mU fU * mC fU * mU fG * mU fC | XXOXOXOXOXOXOXXXXXX | 247 |
| 2421 | UUU | * mC fA * mG * fC * mU * fU * mU * mUmU | O | |
| WV- | AGCUUCUUGUCCAGCUUU | fA * mG fC * mU fU * mC fU * mU fG * mU fC * | XOXOXOXOXOXOXXXXXXO | 248 |
| 2423 | UU | mC fA * mG * fC * mU * fU * mU * mUmU | | |
| WV- | TAGCUUCUUGUC | POT * fA * mG fC * mU fU * mC fU * mU fG * mU | XXOXOXOXOXO | 249 |
| 2424 | CAGCTTTATTGUU | fC * mC fA * G * C * T * T * T * A * T * T * G * | XOXXXXXXXXXXO | |
| | | mUmU | | |
| WV- | TAGCUUCUUGUC | T * fA * mG fC * mU fU * mC fU * mU fG * mU fC | XXOXOXOXOXO | 250 |
| 2425 | CAGCTTTATTGUU | * mC fA * G * C * T * T * T * A * T * T * G * | XOXXXXXXXXXXO | |
| | | mUmU | | |
| WV- | AGCUUCUUGUCCAGCTTT | fA * mG fC * mU fU * mC fU * mU fG * mU fC * | XOXOXOXOXO | 251 |
| 2427 | ATTGUU | mC fA * G * C * T * T * T * A * T * T * G * mUmU | XOXXXXXXXXXXO | |
| WV- | UGGUAATCCACTTTCAGAG | mU * mGmGmUmA * A * T * m5C * m5C * A * | XOOOXXXXXXXXXXXOOOX | 252 |
| 2429 | G | m5C * T * T * T * m5C * mAmGmAmG * mG | | |
| WV- | UAGCUUCUUGUCCAGCUU | POmU * fA * mG fC * mU fU * mC fU * mU fG * | XXOXOXOXOXOXOXXXXO | 253 |
| 2478 | U | mU fC * mC fA * mG * fC * mU * mUmU | | |
| WV- | UAGCUUCUUGUCCAGUU | POmU * fA * mG fC * mU fU * mC fU * mU fG * | XXOXOXOXOXOXOXXO | 254 |
| 2479 | | mU fC * mC fA * mG * mUmU | | |
| WV- | TAGCTUCUTGTCCAGCTUT | POT * fAG * fCT * fUC * fUT * fGT * fCC * fAG * | XOXOXOXOXOXOXOXOXX | 255 |
| 2480 | UU | fCT * fU * T * mUmU | XO | |
| WV- | TAGCTUCTTGTCCAGCTUT | POT * fAG * fCT * fUC * T * T * fGT * fCC * fAG * | XOXOXOXXXOXOXOXOXXX | 256 |
| 2481 | UU | fCT * fU * T * mUmU | O | |
| WV- | TAGCTUCUTGUCCAGCTUT | POT * fAG * fCT * fUC * fUT * fG fU fCC * fAG * | XOXOXOXOXOOOXOXOXX | 257 |
| 2482 | UU | fCT * fU * T * mUmU | XO | |
| WV- | TAGCTUCTTGUCCAGCTUT | POT * fAG * fCT * fUC * T * T * fG fU fCC * fAG * | XOXOXOXXXOOOXOXOXX | 258 |
| 2483 | UU | fCT * fU * T * mUmU | XO | |
| WV- | UAGCUUCUUGUCCAGCUU | mU * fA * mG fC * mU fU * mC fU * mU fG * mU | XXOXOXOXOXOXOXXXXO | 259 |
| 2484 | U | fC * mC fA * mG * fC * mU * mUmU | | |
| WV- | UAGCUUCUUGUCCAGUU | mU * fA * mG fC * mU fU * mC fU * mU fG * mU | XXOXOXOXOXOXOXXO | 260 |
| 2485 | | fC * mC fA * mG * mUmU | | |
| WV- | TAGCTUCUTGTCCAGCTUT | T * fAG * fCT * fUC * fUT * fGT * fCC * fAG * fCT | XOXOXOXOXOXOXOXOXX | 261 |
| 2486 | UU | * fU * T * mUmU | XO | |
| WV- | TAGCTUCTTGTCCAGCTUT | T * fAG * fCT * fUC * T * T * fGT * fCC * fAG * fCT | XOXOXOXXXOXOXOXOXXX | 262 |
| 2487 | UU | * fU * T * mUmU | O | |
| WV- | TAGCTUCUTGUCCAGCTUT | T * fAG * fCT * fUC * fUT * fG fU fCC * fAG * fCT | XOXOXOXOXOOOXOXOXX | 263 |
| 2488 | UU | * fU * T * mUmU | XO | |
| WV- | TAGCTUCTTGUCCAGCTUT | T * fAG * fCT * fUC * T * T * fG fU fCC * fAG * fCT | XOXOXOXXXOOOXOXOXX | 264 |
| 2489 | UU | * fU * T * mUmU | XO | |
| WV- | TAGCTUCUTGTCCAGCTTT | POT * fAG * fCT * fUC * fUT * fGT * fCC * fA * G | XOXOXOXOXO | 265 |
| 2490 | ATTGUU | * C * T * T * T * A * T * T * G * mUmU | XOXXXXXXXXXXXO | |
| WV- | TAGCTUCTTGTCCAGCTTTA | POT * fAG * fCT * fUC * T * T * fGT * fCC * fA * G | XOXOXOXXXO | 266 |
| 2491 | TTGUU | * C * T * T * T * A * T * T * G * mUmU | XOXXXXXXXXXXXO | |
| WV- | TAGCUUCUUGUCCAGCTTT | POT * fA * mG fC * mU fU * mC fU * mU fG * mU | XXOXOXOXOXOXOXXXXXX | 267 |
| 2492 | ATUU | fC * mC fA * G * C * T * T * T * A * T * mUmU | XXO | |
| WV- | TAGCUUCUUGUCCAGCTTT | POT * fA * mG fC * mU fU * mC fU * mU fG * mU | XXOXOXOXOXOXOXXXXXX | 268 |
| 2493 | UU | fC * mC fA * G * C * T * T * T * mUmU | O | |
| WV- | TAGCTUCUTGUCCAGCTTT | POT * fAG * fCT * fUC * fUT * fG fU fCC * fA * G | XOXOXOXOXOOOXXXXXXX | 269 |
| 2494 | ATTGUU | * C * T * T * T * A * T * T * G * mUmU | XXXXO | |
| WV- | TAGCTUCTTGUCCAGCTTT | POT * fAG * fCT * fUC * T * T * fG fU fCC * fA * G | XOXOXOXXXOOOXXXXXXX | 270 |
| 2495 | ATTGUU | * C * T * T * T * A * T * T * G * mUmU | XXXXO | |
| WV- | TAGCTUCUTGTCCAGCTTT | T * fAG * fCT * fUC * fUT * fGT * fCC * fA * G * C | XOXOXOXOXOXOXXXXXXX | 271 |
| 2496 | ATTGUU | * T * T * T * A * T * T * G * mUmU | XXXXO | |
| WV- | TAGCTUCTTGTCCAGCTTTA | T * fAG * fCT * fUC * T * T * fGT * fCC * fA * G * | XOXOXOXXXOXOXXXXXXX | 272 |
| 2497 | TTGUU | C * T * T * T * A * T * T * G * mUmU | XXXXO | |
| WV- | TAGCUUCUUGUCCAGCTTT | T * fA * mG fC * mU fU * mC fU * mU fG * mU fC | XXOXOXOXOXOXOXXXXXX | 273 |
| 2498 | ATUU | * mC fA * G * C * T * T * T * A * T * mUmU | XXO | |
| WV- | TAGCUUCUUGUCCAGCTTT | T * fA * mG fC * mU fU * mC fU * mU fG * mU fC | XXOXOXOXOXOXOXXXXXX | 274 |
| 2499 | UU | * mC fA * G * C * T * T * T * mUmU | O | |
| WV- | TAGCTUCUTGU | T * fAG * fCT * fUC * fUT * fG fU fCC * fA * G * C | XOXOXOXOXOOOXXXXXXX | 275 |
| 2500 | CCAGCTTTATTGUU | * T * T * T * A * T * T * G * mUmU | XXXXO | |
| WV- | TAGCTUCTTGU | T * fAG * fCT * fUC * T * T * fG fU fCC * fA * G * | XOXOXOXXXOOOXXXXXXX | 276 |
| 2501 | CCAGCTTTATTGUU | C * T * T * T * A * T * T * G * mUmU | XXXXO | |
| WV- | TCCUCAGUCUGCUUCGCA | POT * fC * mC fU * mC fA * mG fU * mC fU * mG | XXOXOXOXOXOXOXXXXXX | 277 |
| 2502 | CUU | fC * mU fU * mC * fG * mC * fA * mC * mUmU | O | |
| WV- | TCCUCAGUCUGCTUCGCAC | POT * fCC * fUC * fAG * fUC * fUG * fCT * fUC * | XOXOXOXOXOXOXOXOXO | 278 |
| 2503 | UU | fGC * fAC * mUmU | XO | |
| WV- | TCCUCAGTCUGCTUCGCAC | POT * fCC * fUC * fAG * T * C * fUG * fCT * fUC * | XOXOXOXXXOXOXOXOXO | 279 |
| 2504 | UU | fGC * fAC * mUmU | XO | |
| WV- | TCCUCAGUCUGCTUCGCAC | POT * fCC * fUC * fAG * fUC * fU fG fCT * fUC * | XOXOXOXOXOOOXOXOXO | 280 |
| 2505 | UU | fGC * fAC * mUmU | XO | |
| WV- | TCCUCAGTCUGCTUCGCAC | POT * fCC * fUC * fAG * T * C * fU fG fCT * fUC * | XOXOXOXXXOOOXOXOXO | 281 |
| 2506 | UU | fGC * fAC * mUmU | XO | |
| WV- | TCCUCAGUCUGCUUCGCA | T * fC * mC fU * mC fA * mG fU * mC fU * mG fC | XXOXOXOXOXOXOXXXXXX | 282 |
| 2507 | CUU | * mU fU * mC * fG * mC * fA * mC * mUmU | O | |
| WV- | TCCUCAGUCUGCTUCGCAC | T * fCC * fUC * fAG * fUC * fUG * fCT * fUC * fGC | XOXOXOXOXOXOXOXOXO | 283 |
| 2508 | UU | * fAC * mUmU | XO | |
| WV- | TCCUCAGTCUGCTUCGCAC | T * fCC * fUC * fAG * T * C * fUG * fCT * fUC * | XOXOXOXXXOXOXOXOXO | 284 |
| 2509 | UU | fGC * fAC * mUmU | XO | |
| WV- | TCCUCAGUCUGCTUCGCAC | T * fCC * fUC * fAG * fUC * fU fG fCT * fUC * fGC | XOXOXOXOXOOOXOXOXO | 285 |
| 2510 | UU | * fAC * mUmU | XO | |
| WV- | TCCUCAGTCUGCTUCGCAC | T * fCC * fUC * fAG * T * C * fU fG fCT * fUC * | XOXOXOXXXOOOXOXOXO | 286 |
| 2511 | UU | fGC * fAC * mUmU | XO | |
| WV- | TCCUCAGUCUGC | POT * fC * mC fU * mC fA * mG fU * mC fU * mG | XXOXOXOXOXOXOXXXXXX | 287 |
| 2512 | UUCGCACCTTCUU | fC * mU fU * C * G * C * A * C * C * T * T * C * | XXXXO | |
| | | mUmU | | |
| WV- | TCCUCAGUCUGC | POT * fCC * fUC * fAG * fUC * fUG * fCT * fU * C | XOXOXOXOXOXOXXXXXXX | 288 |
| 2513 | TUCGCACCTTCUU | * G * C * A * C * C * T * T * C * mUmU | XXXXO | |
| WV- | TCCUCAGTCUGC | POT * fCC * fUC * fAG * T * C * fUG * fCT * fU * | XOXOXOXXXOXOXXXXXXX | 289 |
| 2514 | TUCGCACCTTCUU | C * G * C * A * C * C * T * T * C * mUmU | XXXXO | |
| WV- | TCCUCAGUCUGC | POT * fC * mC fU * mC fA * mG fU * mC fU * mG | XXOXOXOXOXOXOXXXXXX | 290 |
| 2515 | UUCGCACCTUU | fC * mU fU * C * G * C * A * C * C * T * mUmU | XXO | |
| WV- | TCCUCAGUCUGCUUCGCA | POT * fC * mC fU * mC fA * mG fU * mC fU * mG | XXOXOXOXOXOXOXXXXXX | 291 |
| 2516 | CUU | fC * mU fU * C * G * C * A * C * mUmU | O | |
| WV- | TCCUCAGUCUGC | POT * fCC * fUC * fAG * fUC * fU fG fCT * fU * C | XOXOXOXOXOOOXXXXXXX | 292 |
| 2517 | TUCGCACCTTCUU | * G * C * A * C * C * T * T * C * mUmU | XXXXO | |
| WV- | TCCUCAGTCUGC | POT * fCC * fUC * fAG * T * C * fU fG fCT * fU * C | XOXOXOXXXOOOXXXXXXX | 293 |
| 2518 | TUCGCACCTTCUU | * G * C * A * C * C * T * T * C * mUmU | XXXXO | |
| WV- | TCCUCAGUCUGC | T * fC * mC fU * mC fA * mG fU * mC fU * mG fC | XXOXOXOXOXOXOXXXXXX | 294 |
| 2519 | UUCGCACCTTCUU | * mU fU * C * G * C * A * C * C * T * T * C * | XXXXO | |
| | | mUmU | | |
| WV- | TCCUCAGUCUGC | T * fCC * fUC * fAG * fUC * fUG * fCT * fU * C * G | XOXOXOXOXOXOXXXXXXX | 295 |
| 2520 | TUCGCACCTTCUU | * C * A * C * C * T * T * C * mUmU | XXXXO | |
| WV- | TCCUCAGTCUGC | T * fCC * fUC * fAG * T * C * fUG * fCT * fU * C * | XOXOXOXXXOXOXXXXXXX | 296 |
| 2521 | TUCGCACCTTCUU | G * C * A * C * C * T * T * C * mUmU | XXXXO | |
| WV- | TCCUCAGUCUGCUUCGCA | T * fC * mC fU * mC fA * mG fU * mC fU * mG fC | XXOXOXOXOXOXOXXXXXX | 297 |
| 2522 | CCTUU | * mU fU * C * G * C * A * C * C * T * mUmU | XXO | |
| WV- | TCCUCAGUCUGCUUCGCA | T * fC * mC fU * mC fA * mG fU * mC fU * mG fC | XXOXOXOXOXOXOXXXXXX | 298 |
| 2523 | CUU | * mU fU * C * G * C * A * C * mUmU | O | |
| WV- | TCCUCAGUCUGC | T * fCC * fUC * fAG * fUC * fU fG fCT * fU * C * G | XOXOXOXOXOOOXXXXXXX | 299 |
| 2524 | TUCGCACCTTCUU | * C * A * C * C * T * T * C * mUmU | XXXXO | |
| WV- | TCCUCAGTCUGC | T * fCC * fUC * fAG * T * C * fU fG fCT * fU * C * | XOXOXOXXXOOOXXXXXXX | 300 |
| 2525 | TUCGCACCTTCUU | G * C * A * C * C * T * T * C * mUmU | XXXXO | |
| WV- | TUGCUUCUUGUCCAGCUU | T * fU * mG fC * mU fU * mC fU * mU fG * mU fC | XXOXOXOXOXOXOXXXXXX | 301 |
| 2547 | UUU | * mC fA * mG * fC * mU * fU * mU * mUmU | O | |
| WV- | TUGCUUCUUGUCCAGCUU | POT * fU * mG fC * mU fU * mC fU * mU fG * mU | XXOXOXOXOXOXOXXXXXX | 302 |
| 2548 | UUU | fC * mC fA * mG * fC * mU * fU * mU * mUmU | O | |
| WV- | AGCUUCTTGTCCAGCUUU | mA * SmGmCmUmU * SC * ST * ST * SG * ST * | SOOOSSSSSSSRSSSOOOS | 303 |
| 2555 | AU | SC * SC * RA * SG * SC * SmUmUmUmA * SmU | | |
| WV- | AGCUUCTTGTCCAGCUUU | mA * SmGmCmUmU * SC * ST * ST * SG * ST * | SOOOSSSSSSRSSSSOOOS | 304 |
| 2556 | AU | SC * RC * SA * SG * SC * SmUmUmUmA * SmU | | |
| WV- | AGCUUCTTGTCCAGCUUU | mA * SmGmCmUmU * SC * ST * ST * SG * RT * | SOOOSSSSRSSSSSSOOOS | 305 |
| 2557 | AU | SC * SC * SA * SG * SC * SmUmUmUmA * SmU | | |
| WV- | AGCUUCTTGTCCAGCUUU | mA * SmGmCmUmU * SC * ST * ST * SG * RT * | SOOOSSSSRSSRSSSOOOS | 306 |
| 2558 | AU | SC * SC * RA * SG * SC * SmUmUmUmA * SmU | | |
| WV- | TUCCAGCUUUAUUAGGGA | POT * fU * mC fC * mA fG * mc fU * mU fU * mA | XXOXOXOXOXOXOXXXXXX | 307 |
| 2621 | CUU | fU * mU fA * mG * fG * mG * fA * mC * mUmU | O | |
| WV- | TUCCAGCUTUAUTAGGGA | POT * fU * C fC * A fG * C fU * T fU * A fU * T fA | XXOXOXOXOXOXOXXXXXX | 308 |
| 2622 | CUU | * G * fG * G * fA * C * mUmU | O | |
| WV- | TGTCCAGCTTTATTGGGAG | TGTCCAGCTTTATTGGGAGG | OOOOOOOOOOOOOOOOO | 309 |
| 2644 | G | | OOO | |
| WV- | UGUCCAGCTTTATTGGGAG | mU * RmGmUmCmC * SA * SG * SC * ST * ST * | ROOOSSSSSSSRSSSOOOR | 310 |
| 2645 | G | ST * SA * RT * ST * SG * SmGmGmAmG * RmG | | |
| WV- | UGUCCAGCTTTATTGGGAG | mU * RmG * RmU * RmC * RmC * SA * SG * SC * | RRRRSSSSSSSRSSSRRRR | 311 |
| 2646 | G | ST * ST * ST * SA * RT * ST * SG * SmG * RmG * | | |
| | | RmA * RmG * RmG | | |
| WV- | UGUCCAGCTTTATTGGGAG | mU * SmG * SmU * SmC * SmC * SA * SG * SC * SSSSSSSSSSSRSSSSSSS | 312 | |
| 2647 | G | ST * ST * ST * SA * RT * ST * SG * SmG * SmG * | | |
| | | SmA * SmG * SmG | | |
| WV- | UGUCCAGCTTTATTGGGAG | fU * S fG fU fC fC * SA * SG * SC * ST * ST * ST * | SOOOSSSSSSSRSSSOOOS | 313 |
| 2648 | G | SA * RT * ST * SG * S fG fG fA fG * S fG | | |
| WV- | UGUCCAGCTTTATTGGGAG | fU * R fG fU fC fC * SA * SG * SC * ST * ST * ST * | ROOOSSSSSSSRSSSOOOR | 314 |
| 2649 | G | SA * RT * ST * SG * S fG fG fA fG * R fG | | |
| WV- | UGUCCAGCTTTATTGGGAG | fU * R fG * R fU * R fC * R fC * SA * SG * SC * ST | RRRRSSSSSSSRSSSRRRR | 315 |
| 2650 | G | * ST * ST * SA * RT * ST * SG * S fG * R fG * R fA | | |
| | | * R fG * R fG | | |
| WV- | UGUCCAGCTTTATTGGGAG | fU * S fG * S fU * S fC * S fC * SA * SG * SC * ST * | SSSSSSSSSSSRSSSSSSS | 316 |
| 2651 | G | ST * ST * SA * RT * ST * SG * S fG * S fG * S fA * S | | |
| | | fG * S fG | | |
| WV- | TAGCUUCUUGUCCAGCUU | PHT * fA * mG fC * mU fU * mC fU * mU fG * mU | XXOXOXOXOXOXOXXXXXX | 317 |
| 2652 | UUU | fC * mC fA * mG * fC * mU * fU * mU * mUmU | O | |
| WV- | TAGCUUCUUGUCCAGCUU | PST * fA * mG fC * mU fU * mC fU * mU fG * mU | XXOXOXOXOXOXOXXXXXX | 318 |
| 2653 | UUU | fC * mC fA * mG * fC * mU * fU * mU * mUmU | O | |
| WV- | TAGCUUCUUGUCCAGCUU | Mod022T * fA * mG fC * mU fU * mC fU * mU fG | OXXOXOXOXOXOXOXXXXX | 319 |
| 2654 | UUU | * mU fC * mC fA * mG * fC * mU * fU * mU * | XO | |
| | | mUmU | | |
| WV- | TAGCUUCUUGUCCAGCUU | Mod022 * T * fA * mG fC * mU fU * mC fU * mU | XXXOXOXOXOXOXOXXXXX | 320 |
| 2655 | UUU | fG * mU fC * mC fA * mG * fC * mU * fU * mU * | XO | |
| | | mUmU | | |
| WV- | AGCUUCUUGUCCAGCUUU | PHMod023 * fA * mG fC * mU fU * mC fU * mU | XXOXOXOXOXOXOXXXXXX | 321 |
| 2656 | UU | fG * mU fC * mC fA * mG * fC * mU * fU * mU * | O | |
| | | mUmU | | |
| WV- | AGCUUCUUGUCCAGCUUU | POMod023 * fA * mG fC * mU fU * mC fU * mU | XXOXOXOXOXOXOXXXXXX | 322 |
| 2657 | UU | fG * mU fC * mC fA * mG * fC * mU * fU * mU * | O | |
| | | mUmU | | |
| WV- | AGCUUCUUGUCCAGCUUU | PSMod023 * fA * mG fC * mU fU * mC fU * mU | XXOXOXOXOXOXOXXXXXX | 323 |
| 2658 | UU | fG * mU fC * mC fA * mG * fC * mU * fU * mU * | O | |
| | | mUmU | | |
| WV- | UGUCCAGCTTTATTGGGAG | L001mU * SmGmUmCmC * SA * SG * SC * ST * | OSOOOSSSSSSRSSSSOOOS | 324 |
| 2679 | G | ST * ST * RA * ST * ST * SG * SmGmGmAmG * | | |
| | | SmG | | |
| WV- | UGUCCAGCTTTATTGGGAG | L001mU * SmGmUmCmC * SA * SG * SC * ST * | OSOOOSSSSSSSSSSRSSOS | 325 |
| 2680 | G | ST * ST * SA * ST * ST * SG * RG * SG * SmAmG * | | |
| | | SmG | | |
| WV- | UGUCCAGCTTTATTGGGAG | L001mU * SmGmUmCmCmA * SG * SC * ST * ST | OSOOOOSSSSSSSSSRSSSS | 326 |
| 2681 | G | * ST * SA * ST * ST * SG * RG * SG * SA * SmG * | | |
| | | SmG | | |
| WV- | TAUAGCAGCUUCUUGUCC | POT * fA * mU fA * mG fC * mA fG * mC fU * mU | XXOXOXOXOXOXOXXXXXX | 327 |
| 2693 | AUU | fC * mU fU * mG * fU * mC * fC * mA * mUmU | O | |
| WV- | TUAGCAGCUUCUUGUCCA | POT * fU * mA fG * mC fA * mG fC * mU fU * mC | XXOXOXOXOXOXOXXXXXX | 328 |
| 2694 | GUU | fU * mU fG * mU * fC * mC * fA * mG * mUmU | O | |
| WV- | AAGCAGCUUCUUGUCCAG | POA * fA * mG fC * mA fG * mC fU * mU fC * mU | XXOXOXOXOXOXOXXXXXX | 329 |
| 2695 | CUU | fU * mG fU * mC * fC * mA * fG * mC * mUmU | O | |
| WV- | TGCAGCUUCUUGUCCAGC | POT * fG * mC fA * mG fC * mU fU * mC fU * mU | XXOXOXOXOXOXOXXXXXX | 330 |
| 2696 | UUU | fG * mU fC * mC * fA * mG * fC * mU * mUmU | O | |
| WV- | TCAGCUUCUUGUCCAGCU | POT * fC * mA fG * mC fU * mU fC * mU fU * mG | XXOXOXOXOXOXOXXXXXX | 331 |
| 2697 | UUU | fU * mC fC * mA * fG * mC * fU * mU * mUmU | O | |
| WV- | TGCUUCUUGUCCAGCUUU | POT * fG * mC fU * mU fC * mU fU * mG fU * mC | XXOXOXOXOXOXOXXXXXX | 332 |
| 2698 | AUU | fC * mA fG * mC * fU * mU * fU * mA * mUmU | O | |
| WV- | TCUUCUUGUCCAGCUUUA | POT * fC * mU fU * mC fU * mU fG * mU fC * mC | XXOXOXOXOXOXOXXXXXX | 333 |
| 2699 | UUU | fA * mG fC * mU * fU * mU * fA * mU * mUmU | O | |
| WV- | TUUCUUGUCCAGCUUUAU | POT * fU * mU fC * mU fU * mG fU * mC fC * mA | XXOXOXOXOXOXOXXXXXX | 334 |
| 2700 | UUU | fG * mC fU * mU * fU * mA * fU * mU * mUmU | O | |
| WV- | AUCUUGUCCAGCUUUAUU | POA * fU * mC fU * mU fG * mU fC * mC fA * mG | XXOXOXOXOXOXOXXXXXX | 335 |
| 2701 | GUU | fC * mU fU * mU * fA * mU * fU * mG * mUmU | O | |
| WV- | ACUUGUCCAGCUUUAUUG | POA * fC * mU fU * mG fU * mC fC * mA fG * mC | XXOXOXOXOXOXOXXXXXX | 336 |
| 2702 | GUU | fU * mU fU * mA * fU * mU * fG * mG * mUmU | O | |
| WV- | TUUGUCCAGCUUUAUUGG | POT * fU * mU fG * mU fC * mC fA * mG fC * mU | XXOXOXOXOXOXOXXXXXX | 337 |
| 2703 | GUU | fU * mU fA * mU * fU * mG * fG * mG * mUmU | O | |
| WV- | AUGUCCAGCUUUAUUGG | POA * fU * mG fU * mC fC * mA fG * mC fU * mU | XXOXOXOXOXOXOXXXXXX | 338 |
| 2704 | GAUU | fU * mA fU * mU * fG * mG * fG * mA * mUmU | O | |
| WV- | UGUCCAGCTTTATTGGGAG | L001mU * mGmUmCmC * A * G * C * T * T * T * | OXOOOXXXXXXXXXXXOOO | 339 |
| 2705 | G | A * T * T * G * mGmGmAmG * mG | X | |
| WV- | UGUCCAGCTTTATTGGGAG | L001mU * mGmUmCmC * A * G * C * T * T * T * | OXOOOXXXXXXXXXXXXXO | 340 |
| 2706 | G | A * T * T * G * G * G * mAmG * mG | X | |
| WV- | UGUCCAGCTTTATTGGGAG | L001mU * mGmUmCmCmA * G * C * T * T * T * | OXOOOXXXXXXXXXXXXX | 341 |
| 2707 | G | A * T * T * G * G * G * A * mG * mG | X | |
| WV- | UGUCCAGCUUUAUUGGG | mU * fG * mU fC * mC fA * mG fC * mU fU * mU | XXOXOXOXOXOXOXXXXXX | 342 |
| 2708 | AGTU | fA * mU fU * mG * fG * mG * fA * mG * T * mU | X | |
| WV- | UGUCCAGCUUUA | mU * fG * mU fC * mC fA * mG fC * mU fU * mU | XXOXOXOXOXOXOXXXXXX | 343 |
| 2709 | UUGGGAGTU | fA * mU fU * mG * fG * mG * fA * mG * AMC6T * | X | |
| | | mU | | |
| WV- | UGUCCAGCUUUA | mU * fG * mU fC * mC fA * mG fC * mU fU * mU | XXOXOXOXOXO | 344 |
| 2710 | UUGGGAGGCCATU | fA * mU fU * G * G * G * A * G * G * C * C * A * T | XOXXXXXXXXXXX | |
| | | * mU | | |
| WV- | UGUCCAGCUUUA | mU * fG * mU fC * mC fA * mG fC * mU fU * mU | XXOXOXOXOXO | 345 |
| 2711 | UUGGGAGGCCATU | fA * mU fU * G * G * G * A * G * G * C * C * A * | XOXXXXXXXXXXX | |
| | | AMC6T * mU | | |
| WV- | UGUCCAGCUUUAUUGGG | POmU * fG * mU fC * mC fA * mG fC * mU fU * | XXOXOXOXOXOXOXXXXO | 346 |
| 2712 | UU | mU fA * mU fU * mG * fG * mG * mUmU | | |
| WV- | UGUCCAGCUUUAUUGGGT | POmU * fG * mU fC * mC fA * mG fC * mU fU * | XXOXOXOXOXOXOXXXXX | 347 |
| 2713 | U | mU fA * mU fU * mG * fG * mG * T * mU | | |
| WV- | UGUCCAGCUUUAUUGUU | POmU * fG * mU fC * mC fA * mG fC * mU fU * | XXOXOXOXOXOXOXXO | 348 |
| 2714 | | mU fA * mU fU * mG * mUmU | | |
| WV- | UGUCCAGCUUUAUUGTU | POmU * fG * mU fC * mC fA * mG fC * mU fU * | XXOXOXOXOXOXOXXX | 349 |
| 2715 | | mU fA * mU fU * mG * T * mU | | |
| WV- | TGTCCAGCTUTATUGGGAG | POT * fGT * fCC * fAG * fCT * fUT * fAT * fUG * | XOXOXOXOXOXOXOXOXX | 350 |
| 2716 | UU | fGG * fA * G * mUmU | XO | |
| WV- | TGTCCAGCTUTATUGGGAG | POT * fGT * fCC * fAG * C * T * fUT * fAT * fUG * | XOXOXOXXXOXOXOXOXXX | 351 |
| 2717 | UU | fGG * fA * G * mUmU | O | |
| WV- | TGTCCAGCTUUATUGGGA | POT * fGT * fCC * fAG * fCT * fU fU fAT * fUG * | XOXOXOXOXOOOXOXOXX | 352 |
| 2718 | GUU | fGG * fA * G * mUmU | XO | |
| WV- | TGTCCAGCTUUATUGGGA | POT * fGT * fCC * fAG * C * T * fU fU fAT * fUG * | XOXOXOXXXOOOXOXOXX | 353 |
| 2719 | GUU | fGG * fA * G * mUmU | XO | |
| WV- | AGUCCAGCUUUAUUGGGA | POA * fG * mU fC * mC fA * mG fC * mU fU * mU | XXOXOXOXOXOXOXXXXXX | 354 |
| 2720 | GUU | fA * mU fU * mG * fG * mG * fA * mG * mUmU | O | |
| WV- | AGUCCAGCUUUAUUGGGA | A * fG * mU fC * mC fA * mG fC * mU fU * mU fA | XXOXOXOXOXOXOXXXXXX | 355 |
| 2721 | GUU | * mU fU * mG * fG * mG * fA * mG * mUmU | O | |
| WV- | UGUCCAGCTTTATTGGGAG | Mod001L001mU * mGmUmCmC * A * G * C * T | OXOOOXXXXXXXXXXXOOO | 356 |
| 2722 | G | * T * T * A * T * T * G * mGmGmAmG * mG | X | |
| WV- | UGUCCAGCTTTATTGGGAG | Mod001L001mU * mGmUmCmC * A * G * C * T | OXOOOXXXXXXXXXXXXXO | 357 |
| 2723 | G | * T * T * A * T * T * G * G * G * mAmG * mG | X | |
| WV- | UGUCCAGCTTTATTGGGAG | Mod001L001mU * mGmUmCmCmA * G * C * T * | OXOOOXXXXXXXXXXXXX | 358 |
| 2724 | G | T * T * A * T * T * G * G * G * A * mG * mG | X | |
| WV- | UGUCCAGCTTTATTGGGAG | Mod001L001mU * SmGmUmCmC * SA * SG * SC | OSOOOSSSSSSRSSSSOOOS | 359 |
| 2725 | G | * ST * ST * ST * RA * ST * ST * SG * | | |
| | | SmGmGmAmG * SmG | | |
| WV- | UGUCCAGCTTTATTGGGAG | Mod001L001mU * SmGmUmCmC * SA * SG * SC | OSOOOSSSSSSSSSSRSSOS | 360 |
| 2726 | G | * ST * ST * ST * SA * ST * ST * SG * RG * SG * | | |
| | | SmAmG * SmG | | |
| WV- | UGUCCAGCTTTATTGGGAG | Mod001L001mU * SmGmUmCmCmA * SG * SC * | OSOOOOSSSSSSSSSRSSSS | 361 |
| 2727 | G | ST * ST * ST * SA * ST * ST * SG * RG * SG * SA * | | |
| | | SmG * SmG | | |
| WV- | UGUCCAGCTTTATTGGGAG | L001mU * mG * mU * mC * mC * A * G * C * T * | OXXXXXXXXXXXXXXXXXXX | 362 |
| 2815 | G | T * T * A * T * T * G * mG * mG * mA * mG * mG | | |
| WV- | UGUCCAGCTTTATTGGGAG | Mod001L001mU * mG * mU * mC * mC * A * G | OXXXXXXXXXXXXXXXXXXX | 363 |
| 2816 | G | * C * T * T * T * A * T * T * G * mG * mG * mA * | | |
| | | mG * mG | | |
| WV- | TGUCCAGCUUUAUUGGGA | T * fG * mU fC * mC fA * mG fC * mU fU * mU fA | XXOXOXOXOXOXOXXXXXX | 364 |
| 2817 | GTU | * mU fU * mG * fG * mG * fA * mG * T * mU | X | |
| WV- | TGUCCAGCUUUAUUGGGA | T * fG * mU fC * mC fA * mG fC * mU fU * mU fA | XXOXOXOXOXOXOXXXXXX | 365 |
| 2818 | GTU | * mU fU * mG * fG * mG * fA * mG * AMC6T * | X | |
| | | mU | | |
| WV- | TGUCCAGCUUUA | T * fG * mU fC * mC fA * mG fC * mU fU * mU fA | XXOXOXOXOXO | 366 |
| 2819 | UUGGGAGGCCATU | * mUfU * G * G * G * A * G * G * C * C * A * T * | XOXXXXXXXXXXX | |
| | | mU | | |
| WV- | TGUCCAGCUUUA | T * fG * mU fC * mC fA * mG fC * mU fU * mU fA | XXOXOXOXOXO | 367 |
| 2820 | UUGGGAGGCCATU | * mUfU * G * G * G * A * G * G * C * C * A * | XOXXXXXXXXXXX | |
| | | AMC6T * mU | | |
| WV- | TGUCCAGCUUUAUUGGGA | POT * fG * mU fC * mC fA * mG fC * mU fU * mU | XXOXOXOXOXOXOXXXXXX | 368 |
| 3021 | GTU | fA * mU fU * mG * fG * mG * fA * mG * T * mU | X | |
| WV- | TGUCCAGCUUUAUUGGGA | T * fG * mU fC * mC fA * mG fC * mU fU * mU fA | XXOXOXOXOXOXOXXXXXX | 369 |
| 3068 | GTU | * mU fU * mG * fG * mG * fA * mG * TGaNC6T * | X | |
| | | mU | | |
| WV- | TGUCCAGCUUUA | T * fG * mU fC * mC fA * mG fC * mU fU * mU fA | XXOXOXOXOXOXOXXXXXX | 370 |
| 3069 | UUGGGAGGCCATU | * mUfU * G * G * G * A * G * G * C * C * A * | XXXXX | |
| | | TGaNC6T * mU | | |
| WV- | UGUCCAGCTTTATTGGGAG | mU * SmGmUmCmC * SA * SG * SC * ST * ST * | SOOOSSSSSSSSDSDOOOS | 371 |
| 3090 | G | ST * SA * ST:T * SG:mGmGmAmG * SmG | | |
| WV- | UGUCCAGCTTTATTGGGAG | mU * SmGmUmCmC * SA * SG * SC * ST * ST * | SOOOSSSSSSSDSDSOOOS | 372 |
| 3091 | G | ST * SA:T * ST:G * SmGmGmAmG * SmG | | |
| WV- | UGUCCAGCTTTATTGGGAG | mU * SmGmUmCmC * SA * SG * SC * ST * ST * | SOOOSSSSSSDSDSSOOOS | 373 |
| 3092 | G | ST:A * ST:T * SG * SmGmGmAmG * SmG | | |
| WV- | TGUCCAGCUUUAUUGGGA | POT * fG * mU fC * mC fA * mG fC * mU fU * mU | XXOXOXOXOXOXOXOOOO | 374 |
| 3122 | GTU | fA * mU fU * mG fGmG fAmG * T * mU | XX | |
| WV- | TGUCCAGCUUUAUUGGGA | POT * S fG * SmU fC * SmC fA * SmG fC * SmU fU | SSOSOSOSOSOSOSOOOOS | 375 |
| 3123 | GTU | * SmU fA * SmU fU * SmG fGmG fAmG * ST * | S | |
| | | SmU | | |
| WV- | TAUAGCAGCUUCUUGUCC | POT * fA * mU fA * mG fC * mA fG * mC fU * mU | XXOXOXOXOXOXOXOOOO | 376 |
| 3124 | ATU | fC * mU fU * mG fUmC fCmA * T * mU | XX | |
| WV- | TUAGCAGCUUCUUGUCCA | POT * fU * mA fG * mC fA * mG fC * mU fU * mC | XXOXOXOXOXOXOXOOOO | 377 |
| 3125 | GTU | fU * mU fG * mU fCmC fAmG * T * mU | XX | |
| WV- | TAGCAGCUUCUUGUCCAG | POT * fA * mG fC * mA fG * mC fU * mU fC * mU | XXOXOXOXOXOXOXOOOO | 378 |
| 3126 | CTU | fU * mG fU * mC fCmA fGmC * T * mU | XX | |
| WV- | TGCAGCUUCUUGUCCAGC | POT * fG * mC fA * mG fC * mU fU * mC fU * mU | XXOXOXOXOXOXOXOOOO | 379 |
| 3127 | UTU | fG * mU fC * mC fAmG fCmU * T * mU | XX | |
| WV- | TCAGCUUCUUGUCCAGCU | POT * fC * mA fG * mC fU * mU fC * mU fU * mG | XXOXOXOXOXOXOXOOOO | 380 |
| 3128 | UTU | fU * mC fC * mA fGmC fUmU * T * mU | XX | |
| WV- | TGCUUCUUGUCCAGCUUU | POT * fG * mC fU * mU fC * mU fU * mG fU * mC | XXOXOXOXOXOXOXOOOO | 381 |
| 3129 | ATU | fC * mA fG * mC fUmU fUmA * T * mU | XX | |
| WV- | TCUUCUUGUCCAGCUUUA | POT * fC * mU fU * mC fU * mU fG * mU fC * mC | XXOXOXOXOXOXOXOOOO | 382 |
| 3130 | UTU | fA * mG fC * mU fUmU fAmU * T * mU | XX | |
| WV- | TUUCUUGUCCAGCUUUAU | POT * fU * mU fC * mU fU * mG fU * mC fC * mA | XXOXOXOXOXOXOXOOOO | 383 |
| 3131 | UTU | fG * mC fU * mU fUmA fUmU * T * mU | XX | |
| WV- | TUCUUGUCCAGCUUUAUU | POT * fU * mC fU * mU fG * mU fC * mC fA * mG | XXOXOXOXOXOXOXOOOO | 384 |
| 3132 | GTU | fC * mU fU * mU fAmU fUmG * T * mU | XX | |
| WV- | TCUUGUCCAGCUUUAUUG | POT * fC * mU fU * mG fU * mC fC * mA fG * mC | XXOXOXOXOXOXOXOOOO | 385 |
| 3133 | GTU | fU * mU fU * mA fUmU fGmG * T * mU | XX | |
| WV- | TUUGUCCAGCUUUAUUGG | POT * fU * mU fG * mU fC * mC fA * mG fC * mU | XXOXOXOXOXOXOXOOOO | 386 |
| 3134 | GTU | fU * mU fA * mU fUmG fGmG * T * mU | XX | |
| WV- | TUGUCCAGCUUUAUUGGG | POT * fU * mG fU * mC fC * mA fG * mC fU * mU | XXOXOXOXOXOXOXOOOO | 387 |
| 3135 | ATU | fU * mA fU * mU fGmG fGmA * T * mU | XX | |
| WV- | TAGCUUCUUGUCCAGCUU | POT * fA * mG fC * mU fU * mC fU * mU fG * mU | XXOXOXOXOXOXOXOOOO | 388 |
| 3136 | UTU | fC * mC fA * mG fCmU fUmU * T * mU | XX | |
| WV- | TGGUCUCAGGCAGCCACG | POT * fG * mG fU * mC fU * mC fA * mG fG * mC | XXOXOXOXOXOXOXOOOO | 389 |
| 3137 | GTU | fA * mG fC * mC fAmC fGmG * T * mU | XX | |
| WV- | TAUAGCAGCUUCUUGUCC | POT * S fA * SmU fA * SmG fC * SmA fG * SmC fU | SSOSOSOSOSOSOSOOOOS | 390 |
| 3138 | ATU | * SmU fC * SmU fU * SmG fUmC fCmA * ST * | S | |
| | | SmU | | |
| WV- | TUAGCAGCUUCUUGUCCA | POT * S fU * SmA fG * SmC fA * SmG fC * SmU | SSOSOSOSOSOSOSOOOOS | 391 |
| 3139 | GTU | fU * SmC fU * SmU fG * SmU fCmC fAmG * ST * | S | |
| | | SmU | | |
| WV- | TAGCAGCUUCUUGUCCAG | POT * S fA * SmG fC * SmA fG * SmC fU * SmU fC | SSOSOSOSOSOSOSOOOOS | 392 |
| 3140 | CTU | * SmU fU * SmG fU * SmC fCmA fGmC * ST * | S | |
| | | SmU | | |
| WV- | TGCAGCUUCUUGUCCAGC | POT * S fG * SmC fA * SmG fC * SmU fU * SmC fU | SSOSOSOSOSOSOSOOOOS | 393 |
| 3141 | UTU | * SmU fG * SmU fC * SmC fAmG fCmU * ST * | S | |
| | | SmU | | |
| WV- | TCAGCUUCUUGUCCAGCU | POT * S fC * SmA fG * SmC fU * SmU fC * SmU fU | SSOSOSOSOSOSOSOOOOS | 394 |
| 3142 | UTU | * SmG fU * SmC fC * SmA fGmC fUmU * ST * | S | |
| | | SmU | | |
| WV- | TGCUUCUUGUCCAGCUUU | POT * S fG * SmC fU * SmU fC * SmU fU * SmG | SSOSOSOSOSOSOSOOOOS | 395 |
| 3143 | ATU | fU * SmC fC * SmA fG * SmC fUmU fUmA * ST * | S | |
| | | SmU | | |
| WV- | TCUUCUUGUCCAGCUUUA | POT * S fC * SmU fU * SmC fU * SmU fG * SmU | SSOSOSOSOSOSOSOOOOS | 396 |
| 3144 | UTU | fC * SmC fA * SmG fC * SmU fUmU fAmU * ST * | S | |
| | | SmU | | |
| WV- | TUUCUUGUCCAGCUUUAU | POT * S fU * SmU fC * SmU fU * SmG fU * SmC | SSOSOSOSOSOSOSOOOOS | 397 |
| 3145 | UTU | fC * SmA fG * SmC fU * SmU fUmA fUmU * ST * | S | |
| | | SmU | | |
| WV- | TUCUUGUCCAGCUUUAUU | POT * S fU * SmC fU * SmU fG * SmU fC * SmC fA | SSOSOSOSOSOSOSOOOOS | 398 |
| 3146 | GTU | * SmG fC * SmU fU * SmU fAmU fUmG * ST * | S | |
| | | SmU | | |
| WV- | TCUUGUCCAGCUUUAUUG | POT * S fC * SmU fU * SmG fU * SmC fC * SmA fG | SSOSOSOSOSOSOSOOOOS | 399 |
| 3147 | GTU | * SmC fU * SmU fU * SmA fUmU fGmG * ST * | S | |
| | | SmU | | |
| WV- | TUUGUCCAGCUUUAUUGG | POT * S fU * SmU fG * SmU fC * SmC fA * SmG fC | SSOSOSOSOSOSOSOOOOS | 400 |
| 3148 | GTU | * SmU fU * SmU fA * SmU fUmG fGmG * ST * | S | |
| | | SmU | | |
| WV- | TUGUCCAGCUUUAUUGGG | POT * S fU * SmG fU * SmC fC * SmA fG * SmC fU | SSOSOSOSOSOSOSOOOOS | 401 |
| 3149 | ATU | * SmU fU * SmA fU * SmU fGmG fGmA * ST * | S | |
| | | SmU | | |
| WV- | TAGCUUCUUGUCCAGCUU | POT * S fA * SmG fC * SmU fU * SmC fU * SmU | SSOSOSOSOSOSOSOOOOS | 402 |
| 3150 | UTU | fG * SmU fC * SmC fA * SmG fCmU fUmU * ST * | S | |
| | | SmU | | |
| WV- | TGGUCUCAGGCAGCCACG | POT * S fG * SmG fU * SmC fU * SmC fA * SmG | SSOSOSOSOSOSOSOOOOS | 403 |
| 3151 | GTU | fG * SmC fA * SmG fC * SmC fAmC fGmG * ST * | S | |
| | | SmU | | |
| WV- | TAGCUUCUUGUCCAGCUU | T * fA * mG fC * mU fU * mC fU * mU fG * mU fC | XXOXOXOXOXOXOXXXXXX | 404 |
| 3242 | UTU | * mC fA * mG * fC * mU * fU * mU * T * mU | X | |
| WV- | TAGCUUCUUGUCCAGCUU | T * fA * mG fC * mU fU * mC fU * mU fG * mU fC | XXOXOXOXOXOXOXXXXXX | 405 |
| 3243 | UTU | * mC fA * mG * fC * mU * fU * mU * TGaNC6T * | X | |
| | | mU | | |
| WV- | TAGCUUCUUGUCCAGCUU | VPT * fA * mG fC * mU fU * mC fU * mU fG * mU | XXOXOXOXOXOXOXXXXXX | 406 |
| 3244 | UTU | fC * mC fA * mG * fC * mU * fU * mU * T * mU | X | |
| WV- | TAGCUUCUUGUCCAGCUU | VPT * fA * mG fC * mU fU * mC fU * mU fG * mU | XXOXOXOXOXOXOXXXXXX | 407 |
| 3245 | UTU | fC * mC fA * mG * fC * mU * fU * mU * TGaNC6T | X | |
| | | * mU | | |
| WV- | TAGCUUCUUGUCCAGCUU | Mod001L001T * fA * mG fC * mU fU * mC fU * | OXXOXOXOXOXOXOXXXXX | 408 |
| 3246 | UTU | mU fG * mU fC * mC fA * mG * fC * mU * fU * | XX | |
| | | mU * T * mU | | |
| WV- | TGUCCAGCUUUAUUGGGA | VPT * fG * mU fC * mC fA * mG fC * mU fU * mU | XXOXOXOXOXOXOXXXXXX | 409 |
| 3247 | GTU | fA * mU fU * mG * fG * mG * fA * mG * T * mU | X | |
| WV- | TGUCCAGCUUUAUUGGGA | VPT * fG * mU fC * mC fA * mG fC * mU fU * mU | XXOXOXOXOXOXOXXXXXX | 410 |
| 3248 | GTU | fA * mU fU * mG * fG * mG * fA * mG * | X | |
| | | TGaNC6T * mU | | |
| WV- | TGUCCAGCUUUAUUGGGA | Mod001L001T * fG * mU fC * mC fA * mG fC * | OXXOXOXOXOXOXOXXXXX | 411 |
| 3249 | GTU | mU fU * mU fA * mU fU * mG * fG * mG * fA * | XX | |
| | | mG * T * mU | | |
| WV- | UGUCCAGCTTTATTGGGAG | fU * mGmUmCmC * A * G * C * T * T * T * A * T | XOOOXXXXXXXXXXXOOOX | 412 |
| 3474 | G | * T * G * mGmGmAmG * fG | | |
| WV- | UGUCCAGCTTTATTGGGAG | fU * fG fU fC fC * A * G * C * T * T * T * A * T * T | XOOOXXXXXXXXXXXOOOX | 413 |
| 3475 | G | * G * fG fG fA fG * fG | | |
| WV- | UGUCCAGCTTTATTGGGAG | fU * mG fU * mC fC * A * G * C * T * T * T * A * T | XOXOXXXXXXXXXXXXOXX | 414 |
| 3476 | G | * T * G * fG * mG fA * mG * fG | | |
| WV- | UGUCCAGCTTTATTGGGAG | fU * mGmUmC fC * A * G * C * T * T * T * A * T | XOOOXXXXXXXXXXXOOOX | 415 |
| 3477 | G | * T * G * fGmGmAmG * fG | | |
| WV- | UGUCCAGCTTTATTGGGAG | fU * fG * fU * fC * fC * A * G * C * T * T * T * A * | XXXXXXXXXXXXXXXXXXX | 416 |
| 3478 | G | T * T * G * fG * fG * fA * fG * fG | | |
| WV- | UGUCCAGCTTTATTGGGAG | fU * SmGmUmCmC * SA * SG * SC * ST * ST * ST | SOOOSSSSSSRSSSSOOOS | 417 |
| 3479 | G | * RA * ST * ST * SG * SmGmGmAmG * S fG | | |
| WV- | UGUCCAGCTTTATTGGGAG | fU * S fG fU fC fC * SA * SG * SC * ST * ST * ST * | SOOOSSSSSSRSSSSOOOS | 418 |
| 3480 | G | RA * ST * ST * SG * S fG fG fA fG * S fG | | |
| WV- | UGUCCAGCTTTATTGGGAG | fU * SmG fU * SmC fC * SA * SG * SC * ST * ST * | SOSOSSSSSSRSSSSSOSS | 419 |
| 3481 | G | ST * RA * ST * ST * SG * S fG * SmG fA * SmG * S | | |
| | | fG | | |
| WV- | UGUCCAGCTTTATTGGGAG | fU * SmGmUmC fC * SA * SG * SC * ST * ST * ST | SOOOSSSSSSRSSSSOOOS | 420 |
| 3482 | G | * RA * ST * ST * SG * S fGmGmAmG * S fG | | |
| WV- | UGUCCAGCTTTATTGGGAG | fU * S fG * S fU * S fC * S fC * SA * SG * SC * ST * | SSSSSSSSSSRSSSSSSSS | 421 |
| 3483 | G | ST * ST * RA * ST * ST * SG * S fG * S fG * S fA * S | | |
| | | fG * S fG | | |
| WV- | UGUCCAGCTTTATTGGGAG | fU * mGmUmCmC * A * G * C * T * T * T * A * T | XOOOXXXXXXXXXXXXXOX | 422 |
| 3484 | G | * T * G * G * G * mAmG * fG | | |
| WV- | UGUCCAGCTTTATTGGGAG | fU * fG fU fC fC * A * G * C * T * T * T * A * T * T | XOOOXXXXXXXXXXXXXOX | 423 |
| 3485 | G | * G * G * G * fA fG * fG | | |
| WV- | UGUCCAGCTTTATTGGGAG | fU * mG fU * mC fC * A * G * C * T * T * T * A * T | XOXOXXXXXXXXXXXXXXX | 424 |
| 3486 | G | * T * G * G * G * fA * mG * fG | | |
| WV- | UGUCCAGCTTTATTGGGAG | fU * mGmUmC fC * A * G * C * T * T * T * A * T | XOOOXXXXXXXXXXXXXOX | 425 |
| 3487 | G | * T * G * G * G * fAmG * fG | | |
| WV- | UGUCCAGCTTTATTGGGAG | fU * fG * fU * fC * fC * A * G * C * T * T * T * A * | XXXXXXXXXXXXXXXXXXX | 426 |
| 3488 | G | T * T * G * G * G * fA * fG * fG | | |
| WV- | UGUCCAGCTTTATTGGGAG | fU * SmGmUmCmC * SA * SG * SC * ST * ST * ST | SOOOSSSSSSSSSSRSSOS | 427 |
| 3489 | G | * SA * ST * ST * SG * RG * SG * SmAmG * S fG | | |
| WV- | UGUCCAGCTTTATTGGGAG | fU * S fG fU fC fC * SA * SG * SC * ST * ST * ST * | SOOOSSSSSSSSSSRSSOS | 428 |
| 3490 | G | SA * ST * ST * SG * RG * SG * S fA fG * S fG | | |
| WV- | UGUCCAGCTTTATTGGGAG | fU * SmG fU * SmC fC * SA * SG * SC * ST * ST * | SOSOSSSSSSSSSSRSSSS | 429 |
| 3491 | G | ST * SA * ST * ST * SG * RG * SG * S fA * SmG * | S | |
| | | fG | | |
| WV- | UGUCCAGCTTTATTGGGAG | fU * SmGmUmC fC * SA * SG * SC * ST * ST * ST | SOOOSSSSSSSSSSRSSOS | 430 |
| 3492 | G | * SA * ST * ST * SG * RG * SG * S fAmG * S fG | | |
| WV- | UGUCCAGCTTTATTGGGAG | fU * S fG * S fU * S fC * S fC * SA * SG * SC * ST * | SSSSSSSSSSSSSSRSSSS | 431 |
| 3493 | G | ST * ST * SA * ST * ST * SG * RG * SG * S fA * S fG | | |
| | | * S fG | | |
| WV- | UGUCCAGCTTTATTGGGAG | fU * mGmUmCmC * A * G * C * T * T * T * A * T | XOOOXXXXXXXXXXXXXXX | 432 |
| 3494 | G | * T * G * G * G * A * mG * fG | | |
| WV- | UGUCCAGCTTTATTGGGAG | fU * fG fU fC fC * A * G * C * T * T * T * A * T * T | XOOOXXXXXXXXXXXXXXX | 433 |
| 3495 | G | * G * G * G * A * fG * fG | | |
| WV- | UGUCCAGCTTTATTGGGAG | fU * mG fU * mc fC * A * G * C * T * T * T * A * T | XOXOXXXXXXXXXXXXXXX | 434 |
| 3496 | G | * T * G * G * G * A * mG * fG | | |
| WV- | UGUCCAGCTTTATTGGGAG | fU * mGmUmC fC * A * G * C * T * T * T * A * T | XOOOXXXXXXXXXXXXXXX | 435 |
| 3497 | G | * T * G * G * G * A * mG * fG | | |
| WV- | UGUCCAGCTTTATTGGGAG | fU * fG * fU * fC * fC * A * G * C * T * T * T * A * | XXXXXXXXXXXXXXXXXXX | 436 |
| 3498 | G | T * T * G * G * G * A * fG * fG | | |
| WV- | UGUCCAGCTTTATTGGGAG | fU * SmGmUmCmC * SA * SG * SC * ST * ST * ST | SOOOSSSSSSSSSSRSSSS | 437 |
| 3499 | G | * SA * ST * ST * SG * RG * SG * SA * SmG * S fG | | |
| WV- | UGUCCAGCTTTATTGGGAG | fU * S fG fU fC fC * SA * SG * SC * ST * ST * ST * | SOOOSSSSSSSSSSRSSSS | 438 |
| 3500 | G | SA * ST * ST * SG * RG * SG * SA * S fG * S fG | | |
| WV- | UGUCCAGCTTTATTGGGAG | fU * SmG fU * SmC fC * SA * SG * SC * ST * ST * | SOSOSSSSSSSSSSRSSSS | 439 |
| 3501 | G | * ST * SA * ST * ST * SG * RG * SG * SA * SmG * S | | |
| | | fG | | |
| WV- | UGUCCAGCTTTATTGGGAG | fU * SmGmUmC fC * SA * SG * SC * ST * ST * ST | SOOOSSSSSSSSSSRSSSS | 440 |
| 3502 | G | * SA * ST * ST * SG * RG * SG * SA * SmG * S fG | | |
| WV- | UGUCCAGCTTTATTGGGAG | fU * S fG * S fU * S fC * S fC * SA * SG * SC * ST * | SSSSSSSSSSSSSSSRSSSS | 441 |
| 3503 | G | ST * ST * SA * ST * ST * SG * RG * SG * SA * S fG | | |
| | | * S fG | | |
| WV- | UGUCCAGCTTTATTGGGAG | fU * mGmUmC fC * A * G * C * T * T * T * A * T | XOOOXXXXXXXXXXXXXXX | 442 |
| 3504 | G | * T * G * G * G * A * fG * fG | | |
| WV- | UGUCCAGCTTTATTGGGAG | fU * SmGmUmC fC * SA * SG * SC * ST * ST * ST | SOOOSSSSSSSSSSRSSSS | 443 |
| 3505 | G | * SA * ST * ST * SG * RG * SG * SA * S fG * S fG | | |
| WV- | TAGCUUCUUGUCCAGCUU | T * fA * mG fC * mU fU * mC fU * mU fG * mU fC | XXOXOXOXOXOXOXXXXXX | 444 |
| 3525 | UTU | * mC fA * mG * fC * mU * fU * mU * AMC6T * | X | |
| | | mU | | |
| WV- | TAGCUUCUUGUCCAGCUU | VPT * fA * mG fC * mU fU * mC fU * mU fG * mU | XXOXOXOXOXOXOXXXXXX | 445 |
| 3526 | UTU | fC * mC fA * mG * fC * mU * fU * mU * AMC6T * | X | |
| | | mU | | |
| WV- | TAGCUUCUUGUCCAGCUU | L001T * fA * mG fC * mU fU * mC fU * mU fG * | OXXOXOXOXOXOXOXXXXX | 446 |
| 3527 | UTU | mU fC * mC fA * mG * fC * mU * fU * mU * T * | XX | |
| | | mU | | |
| WV- | TGUCCAGCUUUAUUGGGA | VPT * fG * mU fC * mC fA * mG fC * mU fU * mU | XXOXOXOXOXOXOXXXXXX | 447 |
| 3528 | GTU | fA * mU fU * mG * fG * mG * fA * mG * AMC6T * | X | |
| | | mU | | |
| WV- | TGUCCAGCUUUAUUGGGA | L001T * fG * mU fC * mC fA * mG fC * mU fU * | OXXOXOXOXOXOXOXXXXX | 448 |
| 3529 | GTU | mU fA * mU fU * mG * fG * mG * fA * mG * T * | XX | |
| | | mU | | |
| WV- | CCUCCCAAUAAAGCUGGA | rC rC rU rC rC rC rA rA rU rA rA rA rG rC rU rG rG | OOOOOOOOOO | 449 |
| 3530 | CA | rA rC rA | OOOOOOOOO | |
| WV- | TGUCCAGCUUUAUUGGGA | PHT * fG * mU fC * mC fA * mG fC * mU fU * mU | XXOXOXOXOXOXOXXXXXX | 450 |
| 3531 | GTU | fA * mU fU * mG * fG * mG * fA * mG * T * mU | X | |
| WV- | TGUCCAGCUUUAUUGGGA | PHT * fG * mU fC * mC fA * mG fC * mU fU * mU | XXOXOXOXOXOXOXXXXXX | 451 |
| 3532 | GTU | fA * mU fU * mG * fG * mG * fA * mG * | X | |
| | | TGaNC6T * mU | | |
| WV- | TGTCCAGCTTTATTGGGAG | Mod001L001Teo * Geo * Teo * Ceo * Ceo * A * | OXXXXXXXXXXXXXXXXXXX | 452 |
| 3533 | G | G * C * T * T * T * A * T * T * G * Geo * Geo * | | |
| | | Aeo * Geo * Geo | | |
| WV- | AGCUUCTTGTCCAGCUUU | Mod001L001mA * mG * mC * mU * mU * C * T * | OXXXXXXXXXXXXXXXXXXX | 453 |
| 3534 | AU | T * G * T * C * C * A * G * C * mU * mU * mU * | | |
| | | mA * mU | | |
| WV- | AGCTTCTTGTCCAGCTTTAT | Mod001L001Aeo * Geo * Ceo * Teo * Teo * C * T | OXXXXXXXXXXXXXXXXXXX | 454 |
| 3535 | | * T * G * T * C * C * A * G * C * Teo * Teo * Teo * | | |
| | | Aeo * Teo | | |
| WV- | CCACCAAGACCGCCAAGGA | rC rC rA rC rC rA rA rG rA rC rC rG rC rC rA rA rG | OOOOOOOOOOOOOOOO | 455 |
| 3795 | UGCAC | rG rA rU rG rC rA rC | OOOOOOO | |
| WV- | ACCGCCAAGGAUGCACUG | rA rC rC rG rC rC rA rA rG rG rA rU rG rC rA rC rU | OOOOOOOOOOOOOOOO | 456 |
| 3796 | AGCAGC | rG rA rG rCA rG rC | OOOOOOO | |
| WV- | AGCAGCGUGCAGGAGUCC | rA rG rC rA rG rC rG rU rG rC rA rG rG rA rG rU rC | OOOOOOOOOOOOOOOO | 457 |
| 3797 | CAGGUG | rC rC rAG rG rU rG | OOOOOOO | |
| WV- | GGAGUCCCAGGUGGCCCA | rG rG rA rG rU rC rC rC rA rG rG rU rG rG rC rC rC | OOOOOOOOOOOOOOOO | 458 |
| 3798 | GCAGGC | rA rG rC rA rG rG rC | OOOOOOO | |
| WV- | CAGGGGCUGGGUGACCGA | rC rA rG rG rG rG rC rU rG rG rG rU rG rA rC rC | OOOOOOOOOOOOOOOO | 459 |
| 3799 | UGGCUU | rG rA rU rG rG rC rU rU | OOOOOOOO | |
| WV- | GGCUGGGUGACCGAUGGC | rG rG rC rU rG rG rG rU rG rA rC rC rG rA rU rG | OOOOOOOOOOOOOOOO | 460 |
| 3800 | UUCAGU | rG rC rU rU rC rA rG rU | OOOOOOO | |
| WV- | CUGGAGCACCGUUAAGGA | rC rU rG rG rA rG rC rA rC rC rG rU rU rA rA rG rG | OOOOOOOOOOOOOOOO | 461 |
| 3801 | CAAGUU | rA rC rA rA rG rU rU | OOOOOOO | |
| WV- | CAGCCGUGGCUGCCUGAG | rC rA rG rC rC rG rU rG rG rC rU rG rC rC rU rG rA | OOOOOOOOOOOOOOOO | 462 |
| 3802 | ACCUCA | rG rA rC rC rU rC rA | OOOOOOO | |
| WV- | GCCGUGGCUGCCUGAGAC | rG rC rC rG rU rG rG rC rU rG rC rC rU rG rA rG rA | OOOOOOOOOOOOOOOO | 463 |
| 3803 | CUCAAU | rC rC rU rC rA rA rU | OOOOOOO | |
| WV- | CUUGGGUCCUGCAAUCUC | rC rU rU rG rG rG rU rC rC rU rG rCA rA rU rC rU | OOOOOOOOOOOOOOOO | 464 |
| 3804 | CAGGGCU | rC rC rA rG rG rG rC rU | OOOOOOOO | |
| WV- | CUGGCCUCCCAAUAAAGC | rC rU rG rG rC rC rU rC rC rC rA rA rU rA rA rA rG | OOOOOOOOOOOOOOOO | 465 |
| 3805 | UGGACA | rC rU rG rG rA rC rA | OOOOOOO | |
| WV- | GGCCUCCCAAUAAAGCUG | rG rG rC rC rU rC rC rC rA rA rU rA rA rA rG rC rU | OOOOOOOOOOOOOOOO | 466 |
| 3806 | GACAAG | rG rG rA rC rA rA rG | OOOOOOO | |
| WV- | AUGCACUGAGCAG | rA rU rG rC rA rC rU rG rA rG rC rA rG rC rG rU rG | OOOOOOOOOOOOO | 467 |
| 3807 | CGUGCAGGAGUCCCAGGU | rC rA rG rG rA rG rU rC rC rC rA rG rG rU rG | OOOOOOOOOOOOOOOO | |
| | G | | OO | |
| WV- | GGCCAGGGGCUGG | rG rG rC rC rA rG rG rG rG rC rU rG rG rG rU rG | OOOOOOOOOOOOO | 468 |
| 3808 | GUGACCGAUGGCUUCAGU | rA rC rC rG rA rU rG rG rC rU rU rC rA rG rU | OOOOOOOOOOOOOOOO | |
| | | O | | |
| WV- | CUUCAGCCGUGGC | rC rU rU rC rA rG rC rC rG rU rG rG rC rU rG rC rC | OOOOOOOOOOOOO | 469 |
| 3809 | UGCCUGAGACCUCAAUA | rU rG rA rG rA rC rC rU rC rA rA rU rA | OOOOOOOOOOOOOOOO | |
| WV- | CUCCUUGGGUCCU | rC rU rC rC rU rU rG rG rG rU rC rC rU rG rC rA rA | OOOOOOOOOOOOO | 470 |
| 3810 | GCAACUCCAGGGCUGC | rC rU rC rC rA rG rG rG rC rU rG rC | OOOOOOOOOOOOOOO | |
| WV- | GCUGGCCUCCCAA | rG rC rU rG rG rC rC rU rC rC rC rA rA rU rA rA rA | OOOOOOOOOOOOO | 471 |
| 3811 | UAAAGCUGGACAAGAAG | rG rC rU rG rG rA rC rA rA rG rA rA rG | OOOOOOOOOOOOOOOO | |
| WV- | TGUCCAGCUUUAUUGGGA | T * fG * mU fC * mC fA * mG fC * mU fU * mU fA | XXOXOXOXOXOXOXXXXXX | 472 |
| 3813 | GUU | * mU fU * mG * fG * mG * fA * mG * mUmU | O | |
| WV- | AGUCCAGCUUUAUUGGGA | A * fG * mU fC * mC fA * mG fC * mU fU * mU fA | XXOXOXOXOXOXOXXXXXX | 473 |
| 3814 | GTU | * mU fU * mG * fG * mG * fA * mG * T * mU | X | |
| WV- | TGUCCAGCUUUAUUGGGA | POT * fG * mU fC * mC fA * mG fC * mU fU * mU | XXOXOXOXOXOXOXXXXXX | 474 |
| 3815 | GUU | fA * mU fU * mG * fG * mG * fA * mG * mUmU | O | |
| WV- | AGUCCAGCUUUAUUGGGA | POA * fG * mU fC * mC fA * mG fC * mU fU * mU | XXOXOXOXOXOXOXXXXXX | 475 |
| 3816 | GTU | fA * mU fU * mG * fG * mG * fA * mG * T * mU | X | |
| WV- | GUCCAGCUUUAUUGGGA | MeOT * fG * mU fC * mC fA * mG fC * mU fU * | XXOXOXOXOXOXOXXXXXX | 476 |
| 3817 | GTU | mU fA * mU fU * mG * fG * mG * fA * mG * T * | X | |
| | | mU | | |
| WV- | GUCCAGCUUUAUUGGGA | IT * fG * mU fC * mC fA * mG fC * mU fU * mU | XXOXOXOXOXOXOXXXXXX | 477 |
| 3818 | GTU | fA * mU fU * mG * fG * mG * fA * mG * T * mU | X | |
| WV- | GUCCAGCUUUAUUGGGA | POIT * fG * mU fC * mC fA * mG fC * mU fU * | XXOXOXOXOXOXOXXXXXX | 478 |
| 3819 | GTU | mU fA * mU fU * mG * fG * mG * fA * mG * T * | | |
| | | mU | | |
| WV- | TGUCCAGCUUUAUUGGGA | PHT * fG * mU fC * mC fA * mG fC * mU fU * mU | XXOXOXOXOXOXOXXXXXX | 479 |
| 3880 | GTU | fA * mU fU * mG * fG * mG * fA * mG * AMC6T * | X | |
| | | mU | | |
| WV- | TGTCCAGCTTTATTGGGAG | Mod001L001Teo * Geo * Teo * m5Ceo * m5Ceo | OXXXXXXXXXXXXXXXXXXX | 480 |
| 3967 | G | * A * G * C * T * T * T * A * T * T * G * Geo * Geo | | |
| | | * Aeo * Geo * Geo | | |
| WV- | AGCTTCTTGTCCAGCTTTAT | Mod001L001Aeo * Geo * m5Ceo * Teo * Teo * C | OXXXXXXXXXXXXXXXXXXX | 481 |
| 3968 | | * T * T * G * T * C * C * A * G * C * Teo * Teo * | | |
| | | Teo * Aeo * Teo | | |
| WV- | TGTCCAGCTTTATTGGGAG | L001Teo * Geo * Teo * m5Ceo * m5Ceo * A * G | OXXXXXXXXXXXXXXXXXXX | 482 |
| 3972 | G | * C * T * T * T * A * T * T * G * Geo * Geo * Aeo | | |
| | | * Geo * Geo | | |
| WV- | AGCTTCTTGTCCAGCTTTAT | L001Aeo * Geo * m5Ceo * Teo * Teo * C * T * T * | OXXXXXXXXXXXXXXXXXXX | 483 |
| 3973 | | G * T * C * C * A * G * C * Teo * Teo * Teo * Aeo | | |
| | | * Teo | | |
| WV- | AGCUUCTTGTCCAGCUUU | L001mA * mG * mC * mU * mU * C * T * T * G * | OXXXXXXXXXXXXXXXXXXX | 484 |
| 3974 | AU | T * C * C * A * G * C * mU * mU * mU * mA * mU | | |
| WV- | GCAUCCTTGGCGGTCUUG | Mod001L001mG * mC * mA * mU * mC * C * T * | OXXXXXXXXXXXXXXXXXXX | 485 |
| 4125 | GU | T * G * G * C * G * G * T * C * mU * mU * mG * | | |
| | | mG * mU | | |
| WV- | UGCUCAGTGCATCCTUGGC | Mod001L001mU * mG * mC * mU * mC * A * G | OXXXXXXXXXXXXXXXXXXX | 486 |
| 4126 | G | * T * G * C * A * T * C * C * T * mU * mG * mG * | | |
| | | mC * mG | | |
| WV- | CCUGGGACTCCTGCACGCU | Mod001L001mC * mC * mU * mG * mG * G * A * | OXXXXXXXXXXXXXXXXXXX | 487 |
| 4127 | G | C * T * C * C * T * G * C * A * mC * mG * mC * | | |
| | | mU * mG | | |
| WV- | CUGCUGGGCCACCTGGGA | Mod001L001mC * mU * mG * mC * mU * G * G | OXXXXXXXXXXXXXXXXXXX | 488 |
| 4128 | CU | * G * C * C * A * C * C * T * G * mG * mG * mA * | | |
| | | mC * mU | | |
| WV- | GCCAUCGGTCACCCAGCCC | Mod001L001mG * mC * mC * mA * mU * C * G * | OXXXXXXXXXXXXXXXXXXX | 489 |
| 4129 | C | G * T * C * A * C * C * C * A * mG * mC * mC * | | |
| | | mC * mC | | |
| WV- | UGAAGCCATCGGTCACCCA | Mod001L001mU * mG * mA * mA * mG * C * C * | OXXXXXXXXXXXXXXXXXXX | 490 |
| 4130 | G | A * T * C * G * G * T * C * A * mC * mC * mC * | | |
| | | mA * mG | | |
| WV- | CUUGUCCTTAACGGTGCUC | Mod001L001mC * mU * mU * mG * mU * C * C * | OXXXXXXXXXXXXXXXXXXX | 491 |
| 4131 | C | T * T * A * A * C * G * G * T * mG * mC * mU * | | |
| | | mC * mC | | |
| WV- | AGGUCTCAGGCAGCCACG | Mod001L001mA * mG * mG * mU * mC * T * C * | OXXXXXXXXXXXXXXXXXXX | 492 |
| 4132 | GC | A * G * G * C * A * G * C * C * mA * mC * mG * | | |
| | | mG * mC | | |
| WV- | UGAGGTCTCAGGCAGCCAC | Mod001L001mU * mG * mA * mG * mG * T * C * | OXXXXXXXXXXXXXXXXXXX | 493 |
| 4133 | G | T * C * A * G * G * C * A * G * mC * mC * mA * | | |
| | | mC * mG | | |
| WV- | CCUGGAGATTGCAGGACCC | Mod001L001mC * mC * mU * mG * mG * A * G * | OXXXXXXXXXXXXXXXXXXX | 494 |
| 4134 | A | A * T * T * G * C * A * G * G * mA * mC * mC * | | |
| | | mC * mA | | |
| WV- | UCCAGCTTTATTGGGAGGC | Mod001L001mU * mC * mC * mA * mG * C * T * | OXXXXXXXXXXXXXXXXXXX | 495 |
| 4135 | C | T * T * A * T * T * G * G * G * mA * mG * mG * | | |
| | | mC * mC | | |
| WV- | CUUGUCCAGCTTTATUGG | Mod001L001mC * mU * mU * mG * mU * C * C * | OXXXXXXXXXXXXXXXXXXX | 496 |
| 4136 | GA | A * G * C * T * T * T * A * T * mU * mG * mG * | | |
| | | mG * mA | | |
| WV- | AGCTTCTTGTCCAGCTTTAT | Aeo * Geo * m5Ceo * Teo * Teo * m5C * T * T * | XXXXXXXXXXXXXXXXXXX | 497 |
| 4137 | | G * T * m5C * m5C * A * G * m5C * Teo * Teo * | | |
| | | Teo * Aeo * Teo | | |
| WV- | TCCAGCUUUAUUGGGAGG | T * fC * mC fA * mG fC * mU fU * mU fA * mU fU | XXOXOXOXOXOXOXXXXXX | 498 |
| 4139 | CTU | * mG fG * mG * fA * mG * fG * mC * T * mU | X | |
| WV- | TCCCUGGAGAUUGCAGGA | T * fC * mC fC * mU fG * mG fA * mG fA * mU fU | XXOXOXOXOXOXOXXXXXX | 499 |
| 4140 | CTU | * mG fC * mA * fG * mG * fA * mC * T * mU | X | |
| WV- | TCUGGAGAUUGCAGGACC | T * fC * mU fG * mG fA * mG fA * mU fU * mG fC | XXOXOXOXOXOXOXXXXXX | 500 |
| 4141 | CTU | * mA fG * mG * fA * mC * fC * mC * T * mU | X | |
| WV- | TGAGGUCUCAGGCAGCCA | T * fG * mA fG * mG fU * mC fU * mC fA * mG fG | XXOXOXOXOXOXOXXXXXX | 501 |
| 4142 | CTU | * mC fA * mG * fC * mC * fA * mC * T * mU | X | |
| WV- | TAGGUCUCAGGCAGCCAC | T * fA * mG fG * mU fC * mU fC * mA fG * mG fC | XXOXOXOXOXOXOXXXXXX | 502 |
| 4143 | GTU | * mA fG * mC * fC * mA * fC * mG * T * mU | X | |
| WV- | TGGUCUCAGGCAGCCACG | T * fG * mG fU * mC fU * mC fA * mG fG * mC fA | XXOXOXOXOXOXOXXXXXX | 503 |
| 4144 | GTU | * mG fC * mC * fA * mC * fG * mG * T * mU | X | |
| WV- | TUCUCAGGCAGCCACGGC | T * fU * mC fU * mC fA * mG fG * mC fA * mG fC | XXOXOXOXOXOXOXXXXXX | 504 |
| 4145 | UTU | * mC fA * mC * fG * mG * fC * mU * T * mU | X | |
| WV- | TCCAUCGGUCACCCAGCCC | T * fC * mC fA * mU fC * mG fG * mU fC * mA fC | XXOXOXOXOXOXOXXXXXX | 505 |
| 4146 | TU | * mC fC * mA * fG * mC * fC * mC * T * mU | X | |
| WV- | TCUGGGACUCCUGCACGC | T * fC * mU fG * mG fG * mA fC * mU fC * mC fU | XXOXOXOXOXOXOXXXXXX | 506 |
| 4147 | UTU | * mG fC * mA * fC * mG * fC * mU * T * mU | X | |
| WV- | TGCUCAGUGCAUCCUUGG | T * fG * mC fU * mC fA * mG fU * mG fC * mA fU | XXOXOXOXOXOXOXXXXXX | 507 |
| 4148 | CTU | * mC fC * mU * fU * mG * fG * mC * T * mU | X | |
| WV- | TGCAUCCUUGGCGGUCUU | T * fG * mC fA * mU fC * mC fU * mU fG * mG fC | XXOXOXOXOXOXOXXXXXX | 508 |
| 4149 | GTU | * mG fG * mU * fC * mU * fU * mG * T * mU | X | |
| WV- | TCCAGCUUUAUUGGGAGG | POT * fC * mC fA * mG fC * mU fU * mU fA * mU | XXOXOXOXOXOXOXXXXXX | 509 |
| 4150 | CTU | fU * mG fG * mG * fA * mG * fG * mC * T * mU | X | |
| WV- | TCCCUGGAGAUUGCAGGA | POT * fC * mC fC * mU fG * mG fA * mG fA * mU | XXOXOXOXOXOXOXXXXXX | 510 |
| 4151 | CTU | fU * mG fC * mA * fG * mG * fA * mC * T * mU | X | |
| WV- | TCUGGAGAUUGCAGGACC | POT * fC * mU fG * mG fA * mG fA * mU fU * mG | XXOXOXOXOXOXOXXXXXX | 511 |
| 4152 | CTU | fC * mA fG * mG * fA * mC * fC * mC * T * mU | X | |
| WV- | TGAGGUCUCAGGCAGCCA | POT * fG * mA fG * mG fU * mC fU * mC fA * mG | XXOXOXOXOXOXOXXXXXX | 512 |
| 4153 | CTU | fG * mC fA * mG * fC * mC * fA * mC * T * mU | X | |
| WV- | TAGGUCUCAGGCAGCCAC | POT * fA * mG fG * mU fC * mU fC * mA fG * mG | XXOXOXOXOXOXOXXXXXX | 513 |
| 4154 | GTU | fC * mA fG * mC * fC * mA * fC * mG * T * mU | X | |
| WV- | TGGUCUCAGGCAGCCACG | POT * fG * mG fU * mC fU * mC fA * mG fG * mC | XXOXOXOXOXOXOXXXXXX | 514 |
| 4155 | GTU | fA * mG fC * mC * fA * mC * fG * mG * T * mU | X | |
| WV- | TUCUCAGGCAGCCACGGC | POT * fU * mC fU * mC fA * mG fG * mC fA * mG | XXOXOXOXOXOXOXXXXXX | 515 |
| 4156 | UTU | fC * mC fA * mC * fG * mG * fC * mU * T * mU | X | |
| WV- | TCCAUCGGUCACCCAGCCC | POT * fC * mC fA * mU fC * mG fG * mU fC * mA | XXOXOXOXOXOXOXXXXXX | 516 |
| 4157 | TU | fC * mC fC * mA * fG * mC * fC * mC * T * mU | X | |
| WV- | TCUGGGACUCCUGCACGC | POT * fC * mU fG * mG fG * mA fC * mU fC * mC | XXOXOXOXOXOXOXXXXXX | 517 |
| 4158 | UTU | fU * mG fC * mA * fC * mG * fC * mU * T * mU | X | |
| WV- | TGCUCAGUGCAUCCUUGG | POT * fG * mC fU * mC fA * mG fU * mG fC * mA | XXOXOXOXOXOXOXXXXXX | 518 |
| 4159 | CTU | fU * mC fC * mU * fU * mG * fG * mC * T * mU | X | |
| WV- | TGCAUCCUUGGCGGUCUU | POT * fG * mC fA * mU fC * mC fU * mU fG * mG | XXOXOXOXOXOXOXXXXXX | 519 |
| 4160 | GTU | fC * mG fG * mU * fC * mU * fU * mG * T * mU | X | |
| WV- | TGUCCAGCUUUAUUGGGA | T * fG * mU fC * mC fA * mG fC * mU fU * mU fA | XXOXOXOXOXOXOXOOOO | 520 |
| 4161 | GTU | * mU fU * mG fGmG fAmG * T * mU | XX | |
| WV- | TCCAGCUUUAUUGGGAGG | T * fC * mC fA * mG fC * mU fU * mU fA * mU fU | XXOXOXOXOXOXOXOOOO | 521 |
| 4162 | CTU | * mG fG * mG fAmG fGmC * T * mU | XX | |
| WV- | TCCCUGGAGAUUGCAGGA | T * fC * mC fC * mU fG * mG fA * mG fA * mU fU | XXOXOXOXOXOXOXOOOO | 522 |
| 4163 | CTU | * mG fC * mA fGmG fAmC * T * mU | XX | |
| WV- | TCUGGAGAUUGCAGGACC | T * fC * mU fG * mG fA * mG fA * mU fU * mG fC | XXOXOXOXOXOXOXOOOO | 523 |
| 4164 | CTU | * mA fG * mG fAmC fCmC * T * mU | XX | |
| WV- | TGAGGUCUCAGGCAGCCA | T * fG * mA fG * mG fU * mC fU * mC fA * mG fG | XXOXOXOXOXOXOXOOOO | 524 |
| 4165 | CTU | * mC fA * mG fCmC fAmC * T * mU | XX | |
| WV- | TAGGUCUCAGGCAGCCAC | T * fA * mG fG * mU fC * mU fC * mA fG * mG fC | XXOXOXOXOXOXOXOOOO | 525 |
| 4166 | GTU | * mA fG * mC fCmA fCmG * T * mU | XX | |
| WV- | TGGUCUCAGGCAGCCACG | T * fG * mG fU * mC fU * mC fA * mG fG * mC fA | XXOXOXOXOXOXOXOOOO | 526 |
| 4167 | GTU | * mG fC * mC fAmC fGmG * T * mU | XX | |
| WV- | TUCUCAGGCAGCCACGGC | T * fU * mC fU * mC fA * mG fG * mC fA * mG fC | XXOXOXOXOXOXOXOOOO | 527 |
| 4168 | UTU | * mC fA * mC fGmG fCmU * T * mU | XX | |
| WV- | TCCAUCGGUCACCCAGCCC | T * fC * mC fA * mU fC * mG fG * mU fC * mA fC | XXOXOXOXOXOXOXOOOO | 528 |
| 4169 | TU | * mC fC * mA fGmC fCmC * T * mU | XX | |
| WV- | TCUGGGACUCCUGCACGC | T * fC * mU fG * mG fG * mA fC * mU fC * mC fU | XXOXOXOXOXOXOXOOOO | 529 |
| 4170 | UTU | * mG fC * mA fCmG fCmU * T * mU | XX | |
| WV- | TGCUCAGUGCAUCCUUGG | T * fG * mC fU * mC fA * mG fU * mG fC * mA fU | XXOXOXOXOXOXOXOOOO | 530 |
| 4171 | CTU | * mC fC * mU fUmG fGmC * T * mU | XX | |
| WV- | TGCAUCCUUGGCGGUCUU | T * fG * mC fA * mU fC * mC fU * mU fG * mG fC | XXOXOXOXOXOXOXOOOO | 531 |
| 4172 | GTU | * mG fG * mU fCmU fUmG * T * mU | XX | |
| WV- | TCCAGCUUUAUUGGGAGG | POT * fC * mC fA * mG fC * mU fU * mU fA * mU | XXOXOXOXOXOXOXOOOO | 532 |
| 4173 | CTU | fU * mG fG * mG fAmG fGmC * T * mU | XX | |
| WV- | TCCCUGGAGAUUGCAGGA | POT * fC * mC fC * mU fG * mG fA * mG fA * mU | XXOXOXOXOXOXOXOOOO | 533 |
| 4174 | CTU | fU * mG fC * mA fGmG fAmC * T * mU | XX | |
| WV- | TCUGGAGAUUGCAGGACC | POT * fC * mU fG * mG fA * mG fA * mU fU * mG | XXOXOXOXOXOXOXOOOO | 534 |
| 4175 | CTU | fC * mA fG * mG fAmC fCmC * T * mU | XX | |
| WV- | TGAGGUCUCAGGCAGCCA | POT * fG * mA fG * mG fU * mC fU * mC fA * mG | XXOXOXOXOXOXOXOOOO | 535 |
| 4176 | CTU | fG * mC fA * mG fCmC fAmC * T * mU | XX | |
| WV- | TAGGUCUCAGGCAGCCAC | POT * fA * mG fG * mU fC * mU fC * mA fG * mG | XXOXOXOXOXOXOXOOOO | 536 |
| 4177 | GTU | fC * mA fG * mC fCmA fCmG * T * mU | XX | |
| WV- | TUCUCAGGCAGCCACGGC | POT * fU * mC fU * mC fA * mG fG * mC fA * mG | XXOXOXOXOXOXOXOOOO | 537 |
| 4178 | UTU | fC * mC fA * mC fGmG fCmU * T * mU | XX | |
| WV- | TCCAUCGGUCACCCAGCCC | POT * fC * mC fA * mU fC * mG fG * mU fC * mA | XXOXOXOXOXOXOXOOOO | 538 |
| 4179 | TU | fC * mC fC * mA fGmC fCmC * T * mU | XX | |
| WV- | TCUGGGACUCCUGCACGC | POT * fC * mU fG * mG fG * mA fC * mU fC * mC | XXOXOXOXOXOXOXOOOO | 539 |
| 4180 | UTU | fU * mG fC * mA fCmG fCmU * T * mU | XX | |
| WV- | TGCUCAGUGCAUCCUUGG | POT * fG * mC fU * mC fA * mG fU * mG fC * mA | XXOXOXOXOXOXOXOOOO | 540 |
| 4181 | CTU | fU * mC fC * mU fUmG fGmC * T * mU | XX | |
| WV- | TGCAUCCUUGGCGGUCUU | POT * fG * mC fA * mU fC * mC fU * mU fG * mG | XXOXOXOXOXOXOXOOOO | 541 |
| 4182 | GTU | fC * mG fG * mU fCmU fUmG * T * mU | XX | |
| WV- | TCACTGAGAATACTGTCCC | POTeo * fC * mA fC * mT fG * mA fG * mA fA * | XXOXOXOXOXOXOXXXXXX | 542 |
| 4183 | AA | mT fA * mC fT * mG * fT * mC * fC * mC * Aeo * | X | |
| | | Aeo | | |
| WV- | TCACTGAGAATACTGTCCC | Teo * fC * mA fC * mT fG * mA fG * mA fA * mT | XXOXOXOXOXOXOXXXXXX | 543 |
| 4184 | AA | fA * mC fT * mG * fT * mC * fC * mC * Aeo * Aeo | X | |
| WV- | TCACUGAGAAUACUGUCC | POT * fC * mA fC * mU fG * mA fG * mA fA * mU | XXOXOXOXOXOXOXXXXXX | 544 |
| 4185 | CTU | fA * mC fU * mG * fU * mC * fC * mC * T * mU | X | |
| WV- | TCACUGAGAAUACUGUCC | T * fC * mA fC * mU fG * mA fG * mA fA * mU fA | XXOXOXOXOXOXOXXXXXX | 545 |
| 4186 | CTU | * mC fU * mG * fU * mC * fC * mC * T * mU | X | |
| WV- | TCACUGAGAAUACUGUCC | VPT * fC * mA fC * mU fG * mA fG * mA fA * mU | XXOXOXOXOXOXOXXXXXX | 546 |
| 4187 | CTU | fA * mC fU * mG * fU * mC * fC * mC * T * mU | X | |
| WV- | TCACUGAGAAUACUGUCC | POT * fC * mA fC * mU fG * mA fG * mA fA * mU | XXOXOXOXOXOXOXOOOO | 547 |
| 4188 | CTU | fA * mC fU * mG fUmC fCmC * T * mU | XX | |
| WV- | TCACUGAGAAUACUGUCC | T * fC * mA fC * mU fG * mA fG * mA fA * mU fA | XXOXOXOXOXOXOXOOOO | 548 |
| 4189 | CTU | * mC fU * mG fUmC fCmC * T * mU | XX | |
| WV- | TCACUGAGAAUACUGUCC | VPT * fC * mA fC * mU fG * mA fG * mA fA * mU | XXOXOXOXOXOXOXOOOO | 549 |
| 4190 | CTU | fA * mC fU * mG fUmC fCmC * T * mU | XX | |
| WV- | GCAUCCTTGGCGGTCUUG | L001mG * mC * mA * mU * mC * C * T * T * G * | OXXXXXXXXXXXXXXXXXXX | 550 |
| 4192 | GU | G * C * G * G * T * C * mU * mU * mG * mG * | | |
| | | mU | | |
| WV- | UGCUCAGTGCATCCTUGGC | L001mU * mG * mC * mU * mC * A * G * T * G * | OXXXXXXXXXXXXXXXXXXX | 551 |
| 4193 | G | C * A * T * C * C * T * mU * mG * mG * mC * mG | | |
| WV- | CCUGGGACTCCTGCACGCU | L001mC * mC * mU * mG * mG * G * A * C * T * | OXXXXXXXXXXXXXXXXXXX | 552 |
| 4194 | G | C * C * T * G * C * A * mC * mG * mC * mU * mG | | |
| WV- | CUGCUGGGCCACCTGGGA | L001mC * mU * mG * mC * mU * G * G * G * C * | OXXXXXXXXXXXXXXXXXXX | 553 |
| 4195 | CU | C * A * C * C * T * G * mG * mG * mA * mC * mU | | |
| WV- | GCCAUCGGTCACCCAGCCC | L001mG * mC * mC * mA * mU * C * G * G * T * | OXXXXXXXXXXXXXXXXXXX | 554 |
| 4196 | C | C * A * C * C * C * A * mG * mC * mC * mC * mC | | |
| WV- | UGAAGCCATCGGTCACCCA | L001mU * mG * mA * mA * mG * C * C * A * T * | OXXXXXXXXXXXXXXXXXXX | 555 |
| 4197 | G | C * G * G * T * C * A * mC * mC * mC * mA * mG | | |
| WV- | CUUGUCCTTAACGGTGCUC | L001mC * mU * mU * mG * mU * C * C * T * T * | OXXXXXXXXXXXXXXXXXXX | 556 |
| 4198 | C | A * A * C * G * G * T * mG * mC * mU * mC * mC | | |
| WV- | AGGUCTCAGGCAGCCACG | L001mA * mG * mG * mU * mC * T * C * A * G * | OXXXXXXXXXXXXXXXXXXX | 557 |
| 4199 | GC | G * C * A * G * C * C * mA * mC * mG * mG * mC | | |
| WV- | UGAGGTCTCAGGCAGCCAC | L001mU * mG * mA * mG * mG * T * C * T * C * | OXXXXXXXXXXXXXXXXXXX | 558 |
| 4200 | G | A * G * G * C * A * G * mC * mC * mA * mC * mG | | |
| WV- | CCUGGAGATTGCAGGACCC | L001mC * mC * mU * mG * mG * A * G * A * T * | OXXXXXXXXXXXXXXXXXXX | 559 |
| 4201 | A | T * G * C * A * G * G * mA * mC * mC * mC * mA | | |
| WV- | UCCAGCTTTATTGGGAGGC | L001mU * mC * mC * mA * mG * C * T * T * T * | OXXXXXXXXXXXXXXXXXXX | 560 |
| 4202 | C | A * T * T * G * G * G * mA * mG * mG * mC * mC | | |
| WV- | CUUGUCCAGCTTTATUGG | L001mC * mU * mU * mG * mU * C * C * A * G * | OXXXXXXXXXXXXXXXXXXX | 561 |
| 4203 | GA | C * T * T * T * A * T * mU * mG * mG * mG * mA | | |
| WV- | GCAUCCTTGGCGGTCUUG | Mod001L001mG * mCmAmUmC * C * T * T * G * | OXOOOXXXXXXXXXXXOOO | 562 |
| 4204 | GU | G * C * G * G * T * C * mUmUmGmG * mU | X | |
| WV- | UGCUCAGTGCATCCTUGGC | Mod001L001mU * mGmCmUmC * A * G * T * G | OXOOOXXXXXXXXXXXOOO | 563 |
| 4205 | G | * C * A * T * C * C * T * mUmGmGmC * mG | X | |
| WV- | CCUGGGACTCCTGCACGCU | Mod001L001mC * mCmUmGmG * G * A * C * T | OXOOOXXXXXXXXXXXOOO | 564 |
| 4206 | G | * C * C * T * G * C * A * mCmGmCmU * mG | X | |
| WV- | CUGCUGGGCCACCTGGGA | Mod001L001mC * mUmGmCmU * G * G * G * C | OXOOOXXXXXXXXXXXOOO | 565 |
| 4207 | CU | * C * A * C * C * T * G * mGmGmAmC * mU | X | |
| WV- | GCCAUCGGTCACCCAGCCC | Mod001L001mG * mCmCmAmU * C * G * G * T | OXOOOXXXXXXXXXXXOOO | 566 |
| 4208 | C | * C * A * C * C * C * A * mGmCmCmC * mC | X | |
| WV- | UGAAGCCATCGGTCACCCA | Mod001L001mU * mGmAmAmG * C * C * A * T | OXOOOXXXXXXXXXXXOOO | 567 |
| 4209 | G | * C * G * G * T * C * A * mCmCmCmA * mG | X | |
| WV- | CUUGUCCTTAACGGTGCUC | Mod001L001mC * mUmUmGmU * C * C * T * T * | OXOOOXXXXXXXXXXXOOO | 568 |
| 4210 | C | A * A * C * G * G * T * mGmCmUmC * mC | X | |
| WV- | AGGUCTCAGGCAGCCACG | Mod001L001mA * mGmGmUmC * T * C * A * G | OXOOOXXXXXXXXXXXOOO | 569 |
| 4211 | GC | * G * C * A * G * C * C * mAmCmGmG * mC | X | |
| WV- | UGAGGTCTCAGGCAGCCAC | Mod001L001mU * mGmAmGmG * T * C * T * C | OXOOOXXXXXXXXXXXOOO | 570 |
| 4212 | G | * A * G * G * C * A * G * mCmCmAmC * mG | X | |
| WV- | CCUGGAGATTGCAGGACCC | Mod001L001mC * mCmUmGmG * A * G * A * T | OXOOOXXXXXXXXXXXOOO | 571 |
| 4213 | A | * T * G * C * A * G * G * mAmCmCmC * mA | X | |
| WV- | UCCAGCTTTATTGGGAGGC | Mod001L001mU * mCmCmAmG * C * T * T * T * | OXOOOXXXXXXXXXXXOOO | 572 |
| 4214 | C | A * T * T * G * G * G * mAmGmGmC * mC | X | |
| WV- | CUUGUCCAGCTTTATUGG | Mod001L001mC * mUmUmGmU * C * C * A * G | OXOOOXXXXXXXXXXXOOO | 573 |
| 4215 | GA | * C * T * T * T * A * T * mUmGmGmG * mA | X | |
| WV- | AUAGCAGCTTCTTGTCCAG | Mod001L001mA * mUmAmGmC * A * G * C * T | OXOOOXXXXXXXXXXXOOO | 574 |
| 4216 | C | * T * C * T * T * G * T * mCmCmAmG * mC | X | |
| WV- | AUAGCAGCTTCTTGTCCAG | Mod001L001mA * mU * mA * mG * mC * A * G * | OXXXXXXXXXXXXXXXXXXX | 575 |
| 4217 | C | C * T * T * C * T * T * G * T * mC * mC * mA * | | |
| | | mG * mC | | |
| WV- | CACCAAGACCGC | rC rA rC rC rA rA rG rA rC rC rG rC rC rA rA rG rG | OOOOOOOOOOOOOO | 576 |
| 4218 | CAAGGATGCACTGAGCAG | rA rT rG rC rA rC rT rG rA rG rC rA rG | OOOOOOOOOOOOOOO | |
| WV- | GCACUGAGCAGC | rG rC rA rC rU rG rA rG rC rA rG rC rG rU rG rC rA | OOOOOOOOOOOOOO | 577 |
| 4219 | GUGCAGGAGUCCCAGGU | rG rG rA rG rU rC rC rC rA rG rG rU | OOOOOOOOOOOOOO | |
| WV- | GCAGGAGUCCCA | rG rC rA rG rG rA rG rU rC rC rC rA rG rG rU rG rG | OOOOOOOOOOOOOO | 578 |
| 4220 | GGUGGCCCAGCAGG | rC rC rC rA rG rC rA rG rG | OOOOOOOOOOO | |
| WV- | GGCCAGGGGCUG | rG rG rC rC rA rG rG rG rG rC rU rG rG rG rU rG | OOOOOOOOOOOOOO | 579 |
| 4221 | GGUGACCGAUGGCUUCAG | rA rC rC rG rA rU rG rG rC rU rU rC rA rG | OOOOOOOOOOOOOOO | |
| WV- | UGGAGCACCGUUAAGGAC | rU rG rG rA rG rC rA rC rC rG rU rU rA rA rG rG rA | OOOOOOOOOOOOOOOO | 580 |
| 4222 | AAGU | rC rA rA rG rU | OOOOO | |
| WV- | UCAGCCGUGGCUGCCUGA | rU rC rA rG rC rC rG rU rG rG rC rU rG rC rC rU rG | OOOOOOOOOOOOOO | 581 |
| 4223 | GACCUCAA | rA rG rA rC rC rU rC rA rA | OOOOOOOOOOO | |
| WV- | UUGGGUCCUGCAAUCUCC | rU rU rG rG rG rU rC rC rU rG rC rA rA rU rC rU rC | OOOOOOOOOOOOOO | 582 |
| 4224 | AGGGCU | rC rA rG rG rG rC rU | OOOOOOOOO | |
| WV- | UGGCCUCCCAA | rU rG rG rC rC rU rC rC rC rA rA rU rA rA rA rG rC | OOOOOOOOOOOOOO | 583 |
| 4225 | UAAAGCUGGACAAGAA | rU rG rG rA rC rA rA rG rA rA | OOOOOOOOOOOO | |
| WV- | AAUAAAGCUGG | rA rA rU rA rA rA rG rC rU rG rG rA rC rA rA rG rA | OOOOOOOOOOOOOO | 584 |
| 4226 | ACAAGAAGCUGCUAU | rA rG rC rU rG rC rU rA rU | OOOOOOOOOOO | |
| WV- | TGTCCAGCTTTATTGGGAG | Mod001L001Teo * Geo * Teo * m5Ceo * m5Ceo | OXXXXXXXXXXXXXXXXXXX | 585 |
| 4227 | G | * A * G * m5C * T * T * T * A * T * T * G * Geo * | | |
| | | Geo * Aeo * Geo * Geo | | |
| WV- | AGCTTCTTGTCCAGCTTTAT | Mod001L001Aeo * Geo * m5Ceo * Teo * Teo * | OXXXXXXXXXXXXXXXXXXX | 586 |
| 4228 | | m5C * T * T * G * T * m5C * m5C * A * G * m5C * | | |
| | | Teo * Teo * Teo * Aeo * Teo | | |
| WV- | TGTCCAGCTTTATTGGGAG | L001Teo * Geo * Teo * m5Ceo * m5Ceo * A * G | OXXXXXXXXXXXXXXXXXXX | 587 |
| 4229 | G | * m5C * T * T * T * A * T * T * G * Geo * Geo * | | |
| | | Aeo * Geo * Geo | | |
| WV- | AGCTTCTTGTCCAGCTTTAT | L001Aeo * Geo * m5Ceo * Teo * Teo * m5C * T * | OXXXXXXXXXXXXXXXXXXX | 588 |
| 4230 | | T * G * T * m5C * m5C * A * G * m5C * Teo * Teo | | |
| | | * Teo * Aeo * Teo | | |
| WV- | TGUCCAGCUUUAUUGGGA | POT * S fG * mU fC * mC fA * mG fC * mU fU * | SXOXOXOXOXOXOXOOOO | 589 |
| 4234 | GTU | mU fA * mU fU * mG fGmG fAmG * T * mU | XX | |
| WV- | TGUCCAGCUUUAUUGGGA | POT * R fG * mU fC * mC fA * mG fC * mU fU * | RXOXOXOXOXOXOXOOOO | 590 |
| 4235 | GTU | mU fA * mU fU * mG fGmG fAmG * T * mU | XX | |
| WV- | TGUCCAGCUUUAUUGGGA | POT fG * mU fC * mC fA * mG fC * mU fU * mU | OXOXOXOXOXOXOXOOOO | 591 |
| 4236 | GTU | fA * mU fU * mG fGmG fAmG * T * mU | XX | |
| WV- | TGUCCAGCUUUAUUGGGA | T * S fG * mU fC * mC fA * mG fC * mU fU * mU | SXOXOXOXOXOXOXOOOO | 592 |
| 4237 | GTU | fA * mU fU * mG fGmG fAmG * T * mU | XX | |
| WV- | TGUCCAGCUUUAUUGGGA | T * R fG * mU fC * mC fA * mG fC * mU fU * mU | RXOXOXOXOXOXOXOOOO | 593 |
| 4238 | GTU | fA * mU fU * mG fGmG fAmG * T * mU | XX | |
| WV- | TGUCCAGCUUUAUUGGGA | T fG * mU fC * mC fA * mG fC * mU fU * mU fA * | OXOXOXOXOXOXOXOOOO | 594 |
| 4239 | GTU | mU fU * mG fGmG fAmG * T * mU | XX | |
| WV- | CACCAAGACCG | rC rA rC rC rA rA rG rA rC rC rG rC rC rA rA rG rG | OOOOOOOOOOOOOO | 595 |
| 4240 | CCAAGGAUGCACUGAGCA | rA rU rG rC rA rC rU rG rA rG rC rA rG | OOOOOOOOOOOOOOO | |
| | | G | | |
| WV- | TCAUCCUUGGCGGUCUUG | T * fC * mA fU * mC fC * mU fU * mG fG * mC fG | XXOXOXOXOXOXOXXXXXX | 596 |
| 4245 | GTU | * mG fU * mC * fU * mU * fG * mG * T * mU | X | |
| WV- | TUGCUGGGCCACCUGGGA | T * fU * mG fC * mU fG * mG fG * mC fC * mA fC | XXOXOXOXOXOXOXXXXXX | 597 |
| 4246 | CTU | * mC fU * mG * fG * mG * fA * mC * T * mU | X | |
| WV- | TGAAGCCAUCGGUCACCCA | T * fG * mA fA * mG fC * mC fA * mU fC * mG fG | XXOXOXOXOXOXOXXXXXX | 598 |
| 4247 | TU | * mU fC * mA * fC * mC * fC * mA * T * mU | X | |
| WV- | TUUGUCCUUAACGGUGCU | T * fU * mU fG * mU fC * mC fU * mU fA * mA fC | XXOXOXOXOXOXOXXXXXX | 599 |
| 4248 | CTU | * mG fG * mU * fG * mC * fU * mC * T * mU | X | |
| WV- | TUUGUCCAGCUUUAUUGG | T * fU * mU fG * mU fC * mC fA * mG fC * mU fU | XXOXOXOXOXOXOXXXXXX | 600 |
| 4249 | GTU | * mU fA * mU * fU * mG * fG * mG * T * mU | X | |
| WV- | TCAUCCUUGGCGGUCUUG | POT * fC * mA fU * mC fC * mU fU * mG fG * mC | XXOXOXOXOXOXOXXXXXX | 601 |
| 4250 | GTU | fG * mG fU * mC * fU * mU * fG * mG * T * mU | X | |
| WV- | TUGCUGGGCCACCUGGGA | POT * fU * mG fC * mU fG * mG fG * mC fC * mA | XXOXOXOXOXOXOXXXXXX | 602 |
| 4251 | CTU | fC * mC fU * mG * fG * mG * fA * mC * T * mU | X | |
| WV- | TGAAGCCAUCGGUCACCCA | POT * fG * mA fA * mG fC * mC fA * mU fC * mG | XXOXOXOXOXOXOXXXXXX | 603 |
| 4252 | TU | fG * mU fC * mA * fC * mC * fC * mA * T * mU | X | |
| WV- | TUUGUCCUUAACGGUGCU | POT * fU * mU fG * mU fC * mC fU * mU fA * mA | XXOXOXOXOXOXOXXXXXX | 604 |
| 4253 | CTU | fC * mG fG * mU * fG * mC * fU * mC * T * mU | X | |
| WV- | TUUGUCCAGCUUUAUUGG | POT * fU * mU fG * mU fC * mC fA * mG fC * mU | XXOXOXOXOXOXOXXXXXX | 605 |
| 4254 | GTU | fU * mU fA * mU * fU * mG * fG * mG * T * mU | X | |
| WV- | TCAUCCUUGGCGGUCUUG | T * fC * mA fU * mC fC * mU fU * mG fG * mC fG | XXOXOXOXOXOXOXOOOO | 606 |
| 4255 | GTU | * mG fU * mC fUmU fGmG * T * mU | XX | |
| WV- | TUGCUGGGCCACCUGGGA | T * fU * mG fC * mU fG * mG fG * mC fC * mA fC | XXOXOXOXOXOXOXOOOO | 607 |
| 4256 | CTU | * mC fU * mG fGmG fAmC * T * mU | XX | |
| WV- | TGAAGCCAUCGGUCACCCA | T * fG * mA fA * mG fC * mC fA * mU fC * mG fG | XXOXOXOXOXOXOXOOOO | 608 |
| 4257 | TU | * mU fC * mA fCmC fCmA * T * mU | XX | |
| WV- | TUUGUCCUUAACGGUGCU | T * fU * mU fG * mU fC * mC fU * mU fA * mA fC | XXOXOXOXOXOXOXOOOO | 609 |
| 4258 | CTU | * mG fG * mU fGmCfUmC * T * mU | XX | |
| WV- | TUUGUCCAGCUUUAUUGG | T * fU * mU fG * mU fC * mC fA * mG fC * mU fU | XXOXOXOXOXOXOXOOOO | 610 |
| 4259 | GTU | * mU fA * mU fUmG fGmG * T * mU | XX | |
| WV- | TCAUCCUUGGCGGUCUUG | POT * fC * mA fU * mC fC * mU fU * mG fG * mC | XXOXOXOXOXOXOXOOOO | 611 |
| 4260 | GTU | fG * mG fU * mC fUmU fGmG * T * mU | XX | |
| WV- | TUGCUGGGCCACCUGGGA | POT * fU * mG fC * mU fG * mG fG * mC fC * mA | XXOXOXOXOXOXOXOOOO | 612 |
| 4261 | CTU | fC * mC fU * mG fGmG fAmC * T * mU | XX | |
| WV- | TGAAGCCAUCGGUCACCCA | POT * fG * mA fA * mG fC * mC fA * mU fC * mG | XXOXOXOXOXOXOXOOOO | 613 |
| 4262 | TU | fG * mU fC * mA fCmC fCmA * T * mU | XX | |
| WV- | TUUGUCCUUAACGGUGCU | POT * fU * mU fG * mU fC * mC fU * mU fA * mA | XXOXOXOXOXOXOXOOOO | 614 |
| 4263 | CTU | fC * mG fG * mU fGmC fUmC * T * mU | XX | |
| WV- | TGUCCAGCUUUAUUGGGA | T * S fG * mU fC * mC fA * mG fC * mU fU * mU | SXOXOXOXOXOXOXXXXXX | 615 |
| 5288 | GTU | fA * mU fU * mG * fG * mG * fA * mG * T * SmU | S | |
| WV- | TAGCUUCUUGUCCAGCUU | T * S fA * mG fC * mU fU * mC fU * mU fG * mU | SXOXOXOXOXOXOXXXXXX | 616 |
| 5289 | UTU | fC * mC fA * mG * fC * mU * fU * mU * T * SmU | S | |
| WV- | TGUCCAGCUUUAUUGGGA | T * S fG * mU fC * mC fA * mG fC * mU fU * mU | SXOXOXOXOXOXOXXXXXX | 617 |
| 5290 | GGCTU | fA * mU fU * mG * fG * mG * fA * mG * fG * mC | XXS | |
| | | * T * SmU | | |
| WV- | TAGCUUCUUGUCCAGCUU | T * S fA * mG fC * mU fU * mC fU * mU fG * mU | SXOXOXOXOXOXOXXXXXX | 618 |
| 5291 | UAUTU | fC * mC fA * mG * fC * mU * fU * mU * fA * mU | XXS | |
| | | * T * SmU | | |
| WV- | TGUCCAGCUUUAUUGGGA | T * fG * mU fC * mC fA * mG fC * mU fU * mU fA | XXOXOXOXOXOXOXXXXXX | 619 |
| 5292 | GTU | * mU fU * mG * fG * mG * fA * mG * T * SmU | S | |
| WV- | TAGCUUCUUGUCCAGCUU | T * fA * mG fC * mU fU * mC fU * mU fG * mU fC | XXOXOXOXOXOXOXXXXXX | 620 |
| 5293 | UTU | * mC fA * mG * fC * mU * fU * mU * T * SmU | S | |
| WV- | TGUCCAGCUUUAUUGGGA | T * fG * mU fC * mC fA * mG fC * mU fU * mU fA | XXOXOXOXOXOXOXXXXXX | 621 |
| 5294 | GGCTU | * mU fU * mG * fG * mG * fA * mG * fG * mC * T | XXS | |
| | | * SmU | | |
| WV- | TAGCUUCUUGUCCAGCUU | T * fA * mG fC * mU fU * mC fU * mU fG * mU fC | XXOXOXOXOXOXOXXXXXX | 622 |
| 5295 | UAUTU | * mC fA * mG * fC * mU * fU * mU * fA * mU * T | XXS | |
| | | * SmU | | |
| WV- | TGUCCAGCUUUAUUGGGA | T * S fG * mU fC * mC fA * mG fC * mU fU * mU | SXOXOXOXOXOXOXXXXXX | 623 |
| 5296 | GTU | fA * mU fU * mG * fG * mG * fA * mG * T * mU | X | |
| WV- | TAGCUUCUUGUCCAGCUU | T * S fA * mG fC * mU fU * mC fU * mU fG * mU | SXOXOXOXOXOXOXXXXXX | 624 |
| 5297 | UTU | fC * mC fA * mG * fC * mU * fU * mU * T * mU | X | |
| WV- | TGUCCAGCUUUAUUGGGA | T * S fG * mU fC * mC fA * mG fC * mU fU * mU | SXOXOXOXOXOXOXXXXXX | 625 |
| 5298 | GGCTU | fA * mU fU * mG * fG * mG * fA * mG * fG * mC | XXX | |
| | | * T * mU | | |
| WV- | TAGCUUCUUGUCCAGCUU | T * S fA * mG fC * mU fU * mC fU * mU fG * mU | SXOXOXOXOXOXOXXXXXX | 626 |
| 5299 | UAUTU | fC * mC fA * mG * fC * mU * fU * mU * fA * mU | XXX | |
| | | * T * mU | | |
| WV- | GUCCAGCUUUAUUGGGA | fG * mU fC * mC fA * mG fC * mU fU * mU fA * | XOXOXOXOXOXOXXXXXXX | 627 |
| 5300 | GTU | mU fU * mG * fG * mG * fA * mG * T * mU | | |
| WV- | AGCUUCUUGUCCAGCUUU | fA * mG fC * mU fU * mC fU * mU fG * mU fC * | XOXOXOXOXOXOXXXXXXX | 628 |
| 5301 | TU | mC fA * mG * fC * mU * fU * mU * T * mU | | |
| WV- | GCAUCCTTGGCGGTCUUG | L001mG * mCmAmUmC * C * T * T * G * G * C * | OXOOOXXXXXXXXXXXOOO | 629 |
| 5711 | GU | G * G * T * C * mUmUmGmG * mU | X | |
| WV- | UGCUCAGTGCATCCTUGGC | L001mU * mGmCmUmC * A * G * T * G * C * A * | OXOOOXXXXXXXXXXXOOO | 630 |
| 5712 | G | T * C * C * T * mUmGmGmC * mG | X | |
| WV- | CCUGGGACTCCTGCACGCU | L001mC * mCmUmGmG * G * A * C * T * C * C * | OXOOOXXXXXXXXXXXOOO | 631 |
| 5713 | G | T * G * C * A * mCmGmCmU * mG | X | |
| WV- | CUGCUGGGCCACCTGGGA | L001mC * mUmGmCmU * G * G * G * C * C * A * | OXOOOXXXXXXXXXXXOOO | 632 |
| 5714 | CU | C * C * T * G * mGmGmAmC * mU | X | |
| WV- | GCCAUCGGTCACCCAGCCC | L001mG * mCmCmAmU * C * G * G * T * C * A * | OXOOOXXXXXXXXXXXOOO | 633 |
| 5715 | C | C * C * C * A * mGmCmCmC * mC | X | |
| WV- | UGAAGCCATCGGTCACCCA | L001mU * mGmAmAmG * C * C * A * T * C * G * | OXOOOXXXXXXXXXXXOOO | 634 |
| 5716 | G | G * T * C * A * mCmCmCmA * mG | X | |
| WV- | CUUGUCCTTAACGGTGCUC | L001mC * mUmUmGmU * C * C * T * T * A * A * | OXOOOXXXXXXXXXXXOOO | 635 |
| 5717 | C | C * G * G * T * mGmCmUmC * mC | X | |
| WV- | AGGUCTCAGGCAGCCACG | L001mA * mGmGmUmC * T * C * A * G * G * C * | OXOOOXXXXXXXXXXXOOO | 636 |
| 5718 | GC | A * G * C * C * mAmCmGmG * mC | X | |
| WV- | UGAGGTCTCAGGCAGCCAC | L001mU * mGmAmGmG * T * C * T * C * A * G * | OXOOOXXXXXXXXXXXOOO | 637 |
| 5719 | G | G * C * A * G * mCmCmAmC * mG | X | |
| WV- | CCUGGAGATTGCAGGACCC | L001mC * mCmUmGmG * A * G * A * T * T * G * | OXOOOXXXXXXXXXXXOOO | 638 |
| 5720 | A | C * A * G * G * mAmCmCmC * mA | X | |
| WV- | UCCAGCTTTATTGGGAGGC | L001mU * mCmCmAmG * C * T * T * T * A * T * | OXOOOXXXXXXXXXXXOOO | 639 |
| 5721 | C | T * G * G * G * mAmGmGmC * mC | X | |
| WV- | CUUGUCCAGCTTTATUGG | L001mC * mUmUmGmU * C * C * A * G * C * T * | OXOOOXXXXXXXXXXXOOO | 640 |
| 5722 | GA | T * T * A * T * mUmGmGmG * mA | X | |
| WV- | AUAGCAGCTTCTTGTCCAG | L001mA * mUmAmGmC * A * G * C * T * T * C * | OXOOOXXXXXXXXXXXOOO | 641 |
| 5723 | C | T * T * G * T * mCmCmAmG * mC | X | |
| WV- | AUAGCAGCTTCTTGTCCAG | L001mA * mU * mA * mG * mC * A * G * C * T * | OXXXXXXXXXXXXXXXXXXX | 642 |
| 5724 | C | T * C * T * T * G * T * mC * mC * mA * mG * mC | | |
| WV- | AGCTTCTTGTCCAGCTTTAT | Mod001L001Aeo * RGeo * Rm5Ceo * RTeo * | ORRRRRSSSSSSRSSRRRRR | 643 |
| 6001 | | RTeo * RC * ST * ST * SG * ST * SC * SC * RA * SG | | |
| | | * SC * RTeo * RTeo * RTeo * RAeo * RTeo | | |
| WV- | AGCTTCTTGTCCAGCTTTAT | Mod001L001Aeo * RGeo * Rm5Ceo * RTeo * | ORRRRRSSSRSSSSSRRRRR | 644 |
| 6002 | | RTeo * RC * ST * ST * SG * RT * SC * SC * SA * SG | | |
| | | * SC * RTeo * RTeo * RTeo * RAeo * RTeo | | |
| WV- | AGCTTCTTGTCCAGCTTTAT | Mod001L001Aeo * RGeo * Rm5Ceo * RTeo * | ORRRRRSSSRSSRSSRRRRR | 645 |
| 6003 | | RTeo * RC * ST * ST * SG * RT * SC * SC * RA * SG | | |
| | | * SC * RTeo * RTeo * RTeo * RAeo * RTeo | | |
| WV- | AGCUTCTTGTCCAGCTUUA | Mod001L001mA * mGmCmUTeo * C * T * T * G | OXOOOXXXXXXXXXXXOOO | 646 |
| 6004 | U | * T * C * C * A * G * C * TeomUmUmA * mU | X | |
| WV- | AGCUTCTTGTCCAGCTUUA | Mod001L001mA * SmGmCmUTeo * RC * ST * ST | OSOOORSSSSSSRSSROOOS | 647 |
| 6005 | U | * SG * ST * SC * SC * RA * SG * SC * | | |
| | | RTeomUmUmA * SmU | | |
| WV- | AGCUTCTTGTCCAGCTUUA | Mod001L001mA * SmGmCmUTeo * RC * ST * ST | OSOOORSSSRSSSSSROOOS | 648 |
| 6006 | U | * SG * RT * SC * SC * SA * SG * SC * | | |
| | | RTeomUmUmA * SmU | | |
| WV- | AGCUTCTTGTCCAGCTUUA | Mod001L001mA * SmGmCmUTeo * RC * ST * ST | OSOOORSSSRSSRSSROOO | 649 |
| 6007 | U | * SG * RT * SC * SC * RA * SG * SC * | S | |
| | | RTeomUmUmA * SmU | | |
| WV- | UGUCCAGCTTTATTGGGAG | Mod001L001mU * mGmUmCm5Ceo * A * G * C | OXOOOXXXXXXXXXXXOOO | 650 |
| 6008 | G | * T * T * T * A * T * T * G * GeomGmAmG * mG | X | |
| WV- | UGUCCAGCTTTATTGGGAG | Mod001L001mU * SmGmUmCm5Ceo * RA * SG | OSOOORSSSSSRSSSROOOS | 651 |
| 6009 | G | * SC * ST * ST * ST * RA * ST * ST * SG * | | |
| | | RGeomGmAmG * SmG | | |
| WV- | AGCTTCTTGTCCAGCTTTAT | L001Aeo * RGeo * Rm5Ceo * RTeo * RTeo * RC * | ORRRRRSSSSSSRSSRRRRR | 652 |
| 6017 | | ST * ST * SG * ST * SC * SC * RA * SG * SC * RTeo | | |
| | | * RTeo * RTeo * RAeo * RTeo | | |
| WV- | AGCTTCTTGTCCAGCTTTAT | L001Aeo * RGeo * Rm5Ceo * RTeo * RTeo * RC * | ORRRRRSSSRSSSSSRRRRR | 653 |
| 6018 | | ST * ST * SG * RT * SC * SC * SA * SG * SC * RTeo | | |
| | | * RTeo * RTeo * RAeo * RTeo | | |
| WV- | AGCTTCTTGTCCAGCTTTAT | L001Aeo * RGeo * Rm5Ceo * RTeo * RTeo * RC * | ORRRRRSSSRSSRSSRRRRR | 654 |
| 6019 | | ST * ST * SG * RT * SC * SC * RA * SG * SC * RTeo | | |
| | | * RTeo * RTeo * RAeo * RTeo | | |
| WV- | AGCUTCTTGTCCAGCTUUA | L001mA * mGmCmUTeo * C * T * T * G * T * C * | OXOOOXXXXXXXXXXXOOO | 655 |
| 6020 | U | C * A * G * C * TeomUmUmA * mU | X | |
| WV- | AGCUTCTTGTCCAGCTUUA | L001mA * SmGmCmUTeo * RC * ST * ST * SG * | OSOOORSSSSSSRSSROOOS | 656 |
| 6021 | U | ST * SC * SC * RA * SG * SC * RTeomUmUmA * | | |
| | | SmU | | |
| WV- | AGCUTCTTGTCCAGCTUUA | L001mA * SmGmCmUTeo * RC * ST * ST * SG * | OSOOORSSSRSSSSSROOOS | 657 |
| 6022 | U | RT * SC * SC * SA * SG * SC * RTeomUmUmA * | | |
| | | SmU | | |
| WV- | AGCUTCTTGTCCAGCTUUA | L001mA * SmGmCmUTeo * RC * ST * ST * SG * | OSOOORSSSRSSRSSROOO | 658 |
| 6023 | U | RT * SC * SC * RA * SG * SC * RTeomUmUmA * | S | |
| | | SmU | | |
| WV- | UGUCCAGCTTTATTGGGAG | L001mU * mGmUmCm5Ceo * A * G * C * T * T * | OXOOOXXXXXXXXXXXOOO | 659 |
| 6024 | G | T * A * T * T * G * GeomGmAmG * mG | X | |
| WV- | UGUCCAGCTTTATTGGGAG | L001mU * SmGmUmCm5Ceo * RA * SG * SC * ST | OSOOORSSSSSRSSSROOOS | 660 |
| 6025 | G | * ST * ST * RA * ST * ST * SG * RGeomGmAmG * | | |
| | | SmG | | |
| WV- | AGCTTCTTGTCCAGCTTTAT | Aeo * RGeo * Rm5Ceo * RTeo * RTeo * RC * ST * | RRRRRSSSSSSRSSRRRRR | 661 |
| 6026 | | ST * SG * ST * SC * SC * RA * SG * SC * RTeo * | | |
| | | RTeo * RTeo * RAeo * RTeo | | |
| WV- | AGCTTCTTGTCCAGCTTTAT | Aeo * RGeo * Rm5Ceo * RTeo * RTeo * RC * ST * | RRRRRSSSRSSSSSRRRRR | 662 |
| 6027 | | ST * SG * RT * SC * SC * SA * SG * SC * RTeo * | | |
| | | RTeo * RTeo * RAeo * RTeo | | |
| WV- | AGCTTCTTGTCCAGCTTTAT | Aeo * RGeo * Rm5Ceo * RTeo * RTeo * RC * ST * | RRRRRSSSRSSRSSRRRRR | 663 |
| 6028 | | ST * SG * RT * SC * SC * RA * SG * SC * RTeo * | | |
| | | RTeo * RTeo * RAeo * RTeo | | |
| WV- | AGCUTCTTGTCCAGCTUUA | mA * mGmCmUTeo * C * T * T * G * T * C * C * A | XOOOXXXXXXXXXXXOOOX | 664 |
| 6029 | U | * G * C * TeomUmUmA * mU | | |
| WV- | AGCUTCTTGTCCAGCTUUA | mA * SmGmCmUTeo * RC * ST * ST * SG * ST | SOOORSSSSSSRSSROOOS | 665 |
| 6030 | U | SC * SC * RA * SG * SC * RTeomUmUmA * SmU | | |
| WV- | AGCUTCTTGTCCAGCTUUA | mA * SmGmCmUTeo * RC * ST * ST * SG * RT * | SOOORSSSRSSSSSROOOS | 666 |
| 6031 | U | SC * SC * SA * SG * SC * RTeomUmUmA * SmU | | |
| WV- | AGCUTCTTGTCCAGCTUUA | mA * SmGmCmUTeo * RC * ST * ST * SG * RT * | SOOORSSSRSSRSSROOOS | 667 |
| 6032 | U | SC * SC * RA * SG * SC * RTeomUmUmA * SmU | | |
| WV- | UGUCCAGCTTTATTGGGAG | mU * mGmUmCm5Ceo * A * G * C * T * T * T * A | XOOOXXXXXXXXXXXOOOX | 668 |
| 6033 | G | * T * T * G * GeomGmAmG * mG | | |
| WV- | UGUCCAGCTTTATTGGGAG | mU * SmGmUmCm5Ceo * RA * SG * SC * ST * ST | SOOORSSSSSRSSSROOOS | 669 |
| 6034 | G | * ST * RA * ST * ST * SG * RGeomGmAmG * SmG | | |
| WV- | TGUCCAGCUUUAUUGGGA | T * S fG * mU fC * mC fA * mG fC * mU fU * mU | SXOXOXOXOXOXOXXXXXX | 670 |
| 6035 | GTU | fA * mU fU * mG * fG * mG * fA * mG * | S | |
| | | TGaNC6T * SmU | | |
| WV- | TAGCUUCUUGUCCAGCUU | T * S fA * mG fC * mU fU * mC fU * mU fG * mU | SXOXOXOXOXOXOXXXXXX | 671 |
| 6036 | UTU | fC * mC fA * mG * fC * mU * fU * mU * TGaNC6T | S | |
| | | * SmU | | |
| WV- | TGUCCAGCUUUA | T * S fG * mU fC * mC fA * mG fC * mU fU * mU | SXOXOXOXOXOXOXXXXXX | 672 |
| 6037 | UUGGGAGGCTU | fA * mU fU * mG * fG * mG * fA * mG * fG * mC | XXS | |
| | | * TGaNC6T * SmU | | |
| WV- | TAGCUUCUUGU | T * S fA * mG fC * mU fU * mC fU * mU fG * mU | SXOXOXOXOXOXOXXXXXX | 673 |
| 6038 | CCAGCUUUAUTU | fC * mC fA * mG * fC * mU * fU * mU * fA * mU | XXS | |
| | | * TGaNC6T * SmU | | |
| WV- | TGUCCAGCUUUAUUGGGA | T * fG * mU fC * mC fA * mG fC * mU fU * mU fA | XXOXOXOXOXOXOXXXXXX | 674 |
| 6039 | GTU | * mU fU * mG * fG * mG * fA * mG * TGaNC6T * | S | |
| | | SmU | | |
| WV- | TAGCUUCUUGUCCAGCUU | T * fA * mG fC * mU fU * mC fU * mU fG * mU fC | XXOXOXOXOXOXOXXXXXX | 675 |
| 6040 | UTU | * mC fA * mG * fC * mU * fU * mU * TGaNC6T * | S | |
| | | SmU | | |
| WV- | TGUCCAGCUUUAUUGGGA | T * fG * mU fC * mC fA * mG fC * mU fU * mU fA | XXOXOXOXOXOXOXXXXXX | 676 |
| 6041 | GGCTU | * mU fU * mG * fG * mG * fA * mG * fG * mC * | XXS | |
| | | TGaNC6T * SmU | | |
| WV- | TAGCUUCUUGUCCAGCUU | T * fA * mG fC * mU fU * mC fU * mU fG * mU fC | XXOXOXOXOXOXOXXXXXX | 677 |
| 6042 | UAUTU | * mC fA * mG * fC * mU * fU * mU * fA * mU * | XXS | |
| | | TGaNC6T * SmU | | |
| WV- | TGUCCAGCUUUAUUGGGA | T * S fG * mU fC * mC fA * mG fC * mU fU * mU | SXOXOXOXOXOXOXXXXXX | 678 |
| 6043 | GTU | fA * mU fU * mG * fG * mG * fA * mG * AMC6T * | S | |
| | | SmU | | |
| WV- | TAGCUUCUUGUCCAGCUU | T * S fA * mG fC * mU fU * mC fU * mU fG * mU | SXOXOXOXOXOXOXXXXXX | 679 |
| 6044 | UTU | fC * mC fA * mG * fC * mU * fU * mU * AMC6T * | S | |
| | | SmU | | |
| WV- | TGUCCAGCUUUAUUGGGA | T * S fG * mU fC * mC fA * mG fC * mU fU * mU | SXOXOXOXOXOXOXXXXXX | 680 |
| 6045 | GGCTU | fA * mU fU * mG * fG * mG * fA * mG * fG * mC | XXS | |
| | | * AMC6T * SmU | | |
| WV- | TAGCUUCUUGUCCAGCUU | T * S fA * mG fC * mU fU * mC fU * mU fG * mU | SXOXOXOXOXOXOXXXXXX | 681 |
| 6046 | UAUTU | fC * mC fA * mG * fC * mU * fU * mU * fA * mU | XXS | |
| | | * AMC6T * SmU | | |
| WV- | TGUCCAGCUUUAUUGGGA | T * fG * mU fC * mC fA * mG fC * mU fU * mU fA | XXOXOXOXOXOXOXXXXXX | 682 |
| 6047 | GTU | * mU fU * mG * fG * mG * fA * mG * AMC6T * | S | |
| | | SmU | | |
| WV- | TAGCUUCUUGUCCAGCUU | T * fA * mG fC * mU fU * mC fU * mU fG * mU fC | XXOXOXOXOXOXOXXXXXX | 683 |
| 6048 | UTU | * mC fA * mG * fC * mU * fU * mU * AMC6T * | S | |
| | | SmU | | |
| WV- | TGUCCAGCUUUAUUGGGA | T * fG * mU fC * mC fA * mG fC * mU fU * mU fA | XXOXOXOXOXOXOXXXXXX | 684 |
| 6049 | GGCTU | * mU fU * mG * fG * mG * fA * mG * fG * mC * | XXS | |
| | | AMC6T * SmU | | |
| WV- | TAGCUUCUUGUCCAGCUU | T * fA * mG fC * mU fU * mC fU * mU fG * mU fC | XXOXOXOXOXOXOXXXXXX | 685 |
| 6050 | UAUTU | * mC fA * mG * fC * mU * fU * mU * fA * mU * | XXS | |
| | | AMC6T * SmU | | |
| WV- | TGUCCAGCUUUAUUGGGA | T * S fG * mU fC * mC fA * mG fC * mU * S fU * | SXOXOXOXSXOXSXXXXXXX | 686 |
| 6205 | GGCTU | mU fA * mU * S fU * mG * fG * mG * fA * mG * | XS | |
| | | fG * mC * TGaNC6T * SmU | | |
| WV- | TGUCCAGCUUUAUUGGGA | T * S fG * mU fC * mC fA * mG fC * mU * S fU * | SXOXOXOXSXOXSXXOOOO | 687 |
| 6206 | GGCTU | mU fA * mU * S fU * mG * fGmG fAmG fGmC * | OSS | |
| | | STGaNC6T * SmU | | |
| WV- | TGUCCAGCUUUAUUGGGA | T * S fG * mU fC * mC fA * mG fC * mU * fU * | SXOXOXOXXXOXXXXXXXXX | 688 |
| 6214 | GGCTU | mU fA * mU * fU * mG * fG * mG * fA * mG * fG | XS | |
| | | * mC * TGaNC6T * SmU | | |
| WV- | TGUCCAGCUUUAUUGGGA | T * S fG * mU fC * mC fA * mG fC * mU * fU * | SXOXOXOXXXOXXXXOOO | 689 |
| 6215 | GGCTU | mU fA * mU * fU * mG * fGmG fAmG fGmC * | OOSS | |
| | | STGaNC6T * SmU | | |
| WV- | TGUCCAGCUUUAUUGGGA | T * S fG * mU fC * mC fA * mG fC * mU * fU * | SXOXOXOXXXOXXXXXXXXX | 690 |
| 6411 | GGCTU | mU fA * mU * fU * mG * fG * mG * fA * mG * fG | XS | |
| | | * mC * T * SmU | | |
| WV- | TGUCCAGCUUUAUUGGGA | T * S fG * mU fC * mC fA * mG fC * mU * S fU * | SXOXOXOXSXOXSXXXXXXX | 691 |
| 6412 | GGCTU | mU fA * mU * S fU * mG * fG * mG * fA * mG * | XS | |
| | | fG * mC * T * SmU | | |
| WV- | TGUCCAGCUUUAUUGGGA | T * S fG * mU fC * mC fA * mG fC * mU * S fU * | SXOXOXOXSXOXOXXXXXX | 692 |
| 6413 | GGCTU | mU fA * mU fU * mG * fG * mG * fA * mG * fG * | XXS | |
| | | mC * T * SmU | | |
| WV- | TGUCCAGCUUUAUUGGGA | T * S fG * mU fC * mC fA * mG fC * mU fU * mU | SXOXOXOXOXOXSXXXXXX | 693 |
| 6414 | GGCTU | fA * mU * S fU * mG * fG * mG * fA * mG * fG * | XXS | |
| | | mC * T * SmU | | |
| WV- | TGUCCAGCUUUAUUGGGA | T * S fG * mU fC * mC fA * mG fC * mU * S fU * | SXOXOXOXSXOXSOXXXXXX | 694 |
| 6415 | GGCTU | mU fA * mU * S fUmG * fG * mG * fA * mG * fG | XS | |
| | | * mC * T * SmU | | |
| WV- | TGUCCAGCTUUATUGGGA | T * S fG * mU fC * mC fA * mG fC * Teo fU * mU | SXOXOXOXOXOXOXXXXXX | 695 |
| 6416 | GGCTU | fA * Teo fU * mG * fG * mG * fA * mG * fG * mC | XXS | |
| | | * T * SmU | | |
| WV- | TGUCCAGCTUUAUUGGGA | T * S fG * mU fC * mC fA * mG fC * Teo fU * mU | SXOXOXOXOXOXOXXXXXX | 696 |
| 6417 | GGCTU | fA * mU fU * mG * fG * mG * fA * mG * fG * mC | XXS | |
| | | * T * SmU | | |
| WV- | TGUCCAGCUUUATUGGGA | T * S fG * mU fC * mC fA * mG fC * mU fU * mU | SXOXOXOXOXOXOXXXXXX | 697 |
| 6418 | GGCTU | fA * Teo fU * mG * fG * mG * fA * mG * fG * mC | XXS | |
| | | * T * SmU | | |
| WV- | TGUCCAGCTUUATUGGGA | T * S fG * mU fC * mC fA * mG fC * Teo fU * mU | SXOXOXOXOXOXOOXXXXX | 698 |
| 6419 | GGCTU | fA * Teo fUmG * fG * mG * fA * mG * fG * mC * | XXS | |
| | | T * SmU | | |
| WV- | TAGCUUCUUGUCCAGCUU | T * S fA * mG fC * mU * fU * mC * fU * mU * fG * | SXOXXXXXXXOXOXXXXXXX | 699 |
| 6420 | UAUTU | mU fC * mC fA * mG * fC * mU * fU * mU * fA * | XS | |
| | | mU * T * SmU | | |
| WV- | TAGCUUCUUGUCCAGCUU | T * S fA * mG fC * mU * S fU * mC * S fU * mU * | SXOXSXSXSXOXOXXXXXXX | 700 |
| 6421 | UAUTU | S fG * mU fC * mC fA * mG * fC * mU * fU * mU | XS | |
| | | * fA * mU * T * SmU | | |
| WV- | TAGCUUCUUGUCCAGCUU | T * S fA * mG fC * mU * S fU * mC fU * mU fG * | SXOXSXOXOXOXOXXXXXX | 701 |
| 6422 | UAUTU | mU fC * mC fA * mG * fC * mU * fU * mU * fA * | XXS | |
| | | mU * T * SmU | | |
| WV- | TAGCUUCUUGUCCAGCUU | T * S fA * mG fC * mU fU * mC * S fU * mU fG * | SXOXOXSXOXOXOXXXXXX | 702 |
| 6423 | UAUTU | mU fC * mC fA * mG * fC * mU * fU * mU * fA * | XXS | |
| | | mU * T * SmU | | |
| WV- | TAGCUUCUUGUCCAGCUU | T * S fA * mG fC * mU fU * mC fU * mU * S fG * | SXOXOXOXSXOXOXXXXXX | 703 |
| 6424 | UAUTU | mU fC * mC fA * mG * fC * mU * fU * mU * fA * | XXS | |
| | | mU * T * SmU | | |
| WV- | TAGCUUCUUGUCCAGCUU | T * S fA * mG fC * mU * S fU * mC * S fU * mU * | SXOXSXSXSXOXOOXXXXXX | 704 |
| 6425 | UAUTU | S fG * mU fC * mC fAmG * fC * mU * fU * mU * | XS | |
| | | fA * mU * T * SmU | | |
| WV- | TAGCTUCUTGUCCAGCUU | T * S fA * mG fC * Teo fU * m5Ceo fU * Teo fG * | SXOXOXOXOXOXOXXXXXX | 705 |
| 6426 | UAUTU | mU fC * mC fA * mG * fC * mU * fU * mU * fA * | XXS | |
| | | mU * T * SmU | | |
| WV- | TAGCTUCUUGUCCAGCUU | T * S fA * mG fC * Teo fU * mC fU * mU fG * mU | SXOXOXOXOXOXOXXXXXX | 706 |
| 6427 | UAUTU | fC * mC fA * mG * fC * mU * fU * mU * fA * mU | XXS | |
| | | * T * SmU | | |
| WV- | TAGCUUCUUGUCCAGCUU | T * S fA * mG fC * mU fU * m5Ceo fU * mU fG * | SXOXOXOXOXOXOXXXXXX | 707 |
| 6428 | UAUTU | mU fC * mC fA * mG * fC * mU * fU * mU * fA * | XXS | |
| | | mU * T * SmU | | |
| WV- | TAGCUUCUTGUCCAGCUU | T * S fA * mG fC * mU fU * mC fU * Teo fG * mU | SXOXOXOXOXOXOXXXXXX | 708 |
| 6429 | UAUTU | fC * mC fA * mG * fC * mU * fU * mU * fA * mU | XXS | |
| | | * T * SmU | | |
| WV- | TAGCTUCUTGUCCAGCUU | T * S fA * mG fC * Teo fU * m5Ceo fU * Teo fG * | SXOXOXOXOXOXOOXXXXX | 709 |
| 6430 | UAUTU | mU fC * mC fAmG * fC * mU * fU * mU * fA * | XXS | |
| | | mU * T * SmU | | |
| WV- | TGUCCAGCUUUAUUGGGA | T * S fG * SmU fC * SmC fA * SmG fC * SmU * S | SSOSOSOSSSOSSSSSSSSSSS | 710 |
| 6431 | GGCTU | fU * SmU fA * SmU * S fU * SmG * S fG * SmG * | | |
| | | S fA * SmG * S fG * SmC * ST * SmU | | |
| WV- | TGUCCAGCUUUAUUGGGA | T * S fG * mU fC * mC fA * mG fC * mU * S fU * | SXOXOXOXSXOXSSXXXXXX | 711 |
| 6432 | GGCTU | mU fA * mU * S fU * SmG * fG * mG * fA * mG * | XS | |
| | | fG * mC * T * SmU | | |
| WV- | TGUCCAGCUUUAUUGGGA | T * S fG * mU fC * mC fA * mG fC * mU * S fU * | SXOXOXOXSXOXSXXXXXXX | 712 |
| 6433 | GGCTU | mU fA * mU * S fU * mG * mG * mG * mA * mG | XS | |
| | | * mG * mC * T * SmU | | |
| WV- | TGUCCAGCUUUAUUGGGA | T * S fG * mU fC * mC fA * mG fC * mU * S fU * | SXOXOXOXSXOXSXOOOO | 713 |
| 6434 | GGCTU | mU fA * mU * S fU * mGmGmGmAmGmGmC * T | OOXS | |
| | | * SmU | | |
| WV- | TGUCCAGCUUUAUUGGGA | T * S fG * mU fC * mC fA * mG fC * mU fU * mU | SXOXOXOXOXOXOXXXXXX | 714 |
| 6435 | GGCTU | fA * mU fU * Geo * fG * Geo * fA * Geo * fG * | XXS | |
| | | m5Ceo * T * SmU | | |
| WV- | TGUCCAGCUUUAUUGGGA | T * S fG * mU fC * mC fA * mG fC * mU * S fU * | SXOXOXOXSXOXSXXXXXXX | 715 |
| 6436 | GGCTU | mU fA * mU * S fU * Geo * fG * Geo * fA * Geo * | XS | |
| | | fG * m5Ceo * T * SmU | | |
| WV- | TGUCCAGCUUUAUUGGGA | T * S fG * mU fC * mC fA * mG fC * mU * S fU * | SXOXOXOXSXOXSXXXXXXX | 716 |
| 6437 | GGCTU | mU fA * mU * S fU * Geo * Geo * Geo * Aeo * | XS | |
| | | Geo * Geo * m5Ceo * T * SmU | | |
| WV- | TGUCCAGCUUUAUUGGGA | T * S fG * mU fC * mC fA * mG fC * mU * S fU * | SXOXOXOXSXOXSXOOOO | 717 |
| 6438 | GGCTU | mU fA * mU * S fU * | OOXS | |
| | | GeoGeoGeoAeoGeoGeom5Ceo * T * SmU | | |
| WV- | TGUCCAGCUUUAUUGGGA | T * fG * mU fC * mC fA * mG fC * mU fU * mU fA | XXOXOXOXOXOXOXXXXXX | 718 |
| 6439 | GGCTU | * mU fU * mG * fG * mG * fA * mG * fG * mC * T | XXX | |
| | | * mU | | |
| WV- | TGUCCAGCUUUAUUGGGA | T * fG * mU fC * mC fA * mG fC * mU fU * mU fA | XXOXOXOXOXOXOXXXXXX | 719 |
| 6440 | GGCTC | * mU fU * mG * fG * mG * fA * mG * fG * mC * T | XXX | |
| | | * mC | | |
| WV- | TGUCCAGCUUUAUUGGGA | T * fG * mU fC * mC fA * mG fC * mU fU * mU fA | XXOXOXOXOXOXOXXXXXX | 720 |
| 6441 | GGCTA | * mU fU * mG * fG * mG * fA * mG * fG * mC * T | XXX | |
| | | * mA | | |
| WV- | TGUCCAGCUUUAUUGGGA | T * fG * mU fC * mC fA * mG fC * mU fU * mU fA | XXOXOXOXOXOXOXXXXXX | 721 |
| 6442 | GGCTG | * mU fU * mG * fG * mG * fA * mG * fG * mC * T | XXX | |
| | | * mG | | |
| WV- | TGUCCAGCUUUAUUGGGA | T * fG * mUmC * mC fA * mG fC * mU fU * mU | XXOXOXOXOXOXOXXXXXX | 722 |
| 6443 | GGCTU | fA * mU fU * mG * fG * mG * fA * mG * fG * mC | XXX | |
| | | * T * mU | | |
| WV- | TGUCCAGCUUUAUUGGGA | T * fG * mU fC * mCmA * mG fC * mU fU * mU | XXOXOXOXOXOXOXXXXXX | 723 |
| 6444 | GGCTU | fA * mU fU * mG * fG * mG * fA * mG * fG * mC | XXX | |
| | | * T * mU | | |
| WV- | TGUCCAGCUUUAUUGGGA | T * fG * mU fC * mC fA * mGmC * mU fU * mU | XXOXOXOXOXOXOXXXXXX | 724 |
| 6445 | GGCTU | fA * mU fU * mG * fG * mG * fA * mG * fG * mC | XXX | |
| | | * T * mU | | |
| WV- | TGUCCAGCUUUAUUGGGA | T * fG * mU fC * mC fA * mG fC * fU fU * mU fA * | XXOXOXOXOXOXOXXXXXX | 725 |
| 6446 | GGCTU | mU fU * mG * fG * mG * fA * mG * fG * mC * T * | XXX | |
| | | mU | | |
| WV- | TGUCCAGCUUUAUUGGGA | T * fG * mU fC * mC fA * mG fC * mUmU * mU | XXOXOXOXOXOXOXXXXXX | 726 |
| 6447 | GGCTU | fA * mU fU * mG * fG * mG * fA * mG * fG * mC | XXX | |
| | | * T * mU | | |
| WV- | TGUCCAGCUUUAUUGGGA | T * fG * mU fC * mC fA * mG fC * mU fU * fU fA * | XXOXOXOXOXOXOXXXXXX | 727 |
| 6448 | GGCTU | mU fU * mG * fG * mG * fA * mG * fG * mC * T * | XXX | |
| | | mU | | |
| WV- | TGUCCAGCUUUAUUGGGA | T * fG * mU fC * mC fA * mG fC * mU fU * | XXOXOXOXOXOXOXXXXXX | 728 |
| 6449 | GGCTU | mUmA * mU fU * mG * fG * mG * fA * mG * fG * | XXX | |
| | | mC * T * mU | | |
| WV- | TGUCCAGCUUUAUUGGGA | T * fG * mU fC * mC fA * mG fC * mU fU * mU fA | XXOXOXOXOXOXOXXXXXX | 729 |
| 6450 | GGCTU | * fU fU * mG * fG * mG * fA * mG * fG * mC * T | XXX | |
| | | * mU | | |
| WV- | TGUCCAGCUUUAUUGGGA | T * fG * mU fC * mC fA * mG fC * mU fU * mU fA | XXOXOXOXOXOXOXXXXXX | 730 |
| 6451 | GGCTU | * mU fU * fG * fG * mG * fA * mG * fG * mC * T | XXX | |
| | | * mU | | |
| WV- | TGUCCAGCUUUAUUGGGA | T * fG * mU fC * mC fA * mG fC * mU fU * mU fA | XXOXOXOXOXOXOXXXXXX | 731 |
| 6452 | GGCTU | * mU fU * mG * mG * mG * fA * mG * fG * mC * | XXX | |
| | | T * mU | | |
| WV- | TGUCCAGCUUUAUUGGGA | T * fG * mU fC * mC fA * mG fC * mU fU * mU fA | XXOXOXOXOXOXOXXXXXX | 732 |
| 6453 | GGCTU | * mU fU * mG * fG * fG * fA * mG * fG * mC * T | XXX | |
| | | * mU | | |
| WV- | TGUCCAGCUUUAUUGGGA | T * fG * mU fC * mC fA * mG fC * mU fU * mU fA | XXOXOXOXOXOXOXXXXXX | 733 |
| 6454 | GGCTU | * mU fU * mG * fG * mG * mA * mG * fG * mC * | XXX | |
| | | T * mU | | |
| WV- | TGUCCAGCUUUAUUGGGA | T * fG * mU fC * mC fA * mG fC * mU fU * mU fA | XXOXOXOXOXOXOXXXXXX | 734 |
| 6455 | GGCTU | * mU fU * mG * fG * mG * fA * fG * fG * mC * T | XXX | |
| | | * mU | | |
| WV- | TGUCCAGCUUUAUUGGGA | T * fG * mU fC * mC fA * mG fC * mU fU * mU fA | XXOXOXOXOXOXOXXXXXX | 735 |
| 6456 | GGCTU | * mU fU * mG * fG * mG * fA * mG * mG * mC * | XXX | |
| | | T * mU | | |
| WV- | TGUCCAGCUUUAUUGGGA | T * fG * mU fC * mC fA * mG fC * mU fU * mU fA | XXOXOXOXOXOXOXXXXXX | 736 |
| 6457 | GGCTU | * mU fU * mG * fG * mG * fA * mG * fG * fC * T | XXX | |
| | | * mU | | |
| WV- | TGUCCAGCUUUAUUGGGA | T * S fG * SmU fCmC fAmG fCmU fUmU fAmU | SSOOOOOOOOOOOOOOO | 737 |
| 6458 | GGCTU | fUmG fGmG fAmG fGmC * ST * SmU | OOOSS | |
| WV- | TGUCCAGCUUUAUUGGGA | T * S fG * SmU fCmC fAmG fCmU fUmU fAmU | SSOOOOOOOOOOOOOOO | 738 |
| 6459 | GGCTU | fUmG fGmG fAmG fGmCT * SmU | OOOOS | |
| WV- | TGUCCAGCUUUAUUGGGA | T * S fGmU * S fCmC fAmG fCmU fUmU fAmU | SOSOOOOOOOOOOOOOO | 739 |
| 6460 | GGCTU | fUmG fGmG fAmG fGmCT * SmU | OOOOS | |
| WV- | TGUCCAGCUUUAUUGGGA | T * S fGmU fC * SmC fAmG fCmU fUmU fAmU | SOOSOOOOOOOOOOOOO | 740 |
| 6461 | GGCTU | fUmG fGmG fAmG fGmCT * SmU | OOOOS | |
| WV- | TGUCCAGCUUUAUUGGGA | T * S fGmU fCmC * S fAmG fCmU fUmU fAmU | SOOOSOOOOOOOOOOOO | 741 |
| 6462 | GGCTU | fUmG fGmG fAmG fGmCT * SmU | OOOOS | |
| WV- | TGUCCAGCUUUAUUGGGA | T * S fGmU fCmC fA * SmG fCmU fUmU fAmU | SOOOOSOOOOOOOOOOO | 742 |
| 6463 | GGCTU | fUmG fGmG fAmG fGmCT * SmU | OOOOS | |
| WV- | TGUCCAGCUUUAUUGGGA | T * S fGmU fCmC fAmG * S fCmU fUmU fAmU | SOOOOOSOOOOOOOOOO | 743 |
| 6464 | GGCTU | fUmG fGmG fAmG fGmCT * SmU | OOOOS | |
| WV- | TGUCCAGCUUUAUUGGGA | T * S fGmU fCmC fAmG fC * SmU fUmU fAmU | SOOOOOOSOOOOOOOOO | 744 |
| 6465 | GGCTU | fUmG fGmG fAmG fGmCT * SmU | OOOOS | |
| WV- | TGUCCAGCUUUAUUGGGA | T * S fGmU fCmC fAmG fCmU * S fUmU fAmU | SOOOOOOOSOOOOOOOO | 745 |
| 6466 | GGCTU | fUmG fGmG fAmG fGmCT * SmU | OOOOS | |
| WV- | TGUCCAGCUUUAUUGGGA | T * S fGmU fCmC fAmG fCmU fU * SmU fAmU | SOOOOOOOOSOOOOOOO | 746 |
| 6467 | GGCTU | fUmG fGmG fAmG fGmCT * SmU | OOOOS | |
| WV- | TGUCCAGCUUUAUUGGGA | T * S fGmU fCmC fAmG fCmU fUmU * S fAmU | SOOOOOOOOOSOOOOOO | 747 |
| 6468 | GGCTU | fUmG fGmG fAmG fGmCT * SmU | OOOOS | |
| WV- | TGUCCAGCUUUAUUGGGA | T * S fGmU fCmC fAmG fCmU fUmU fA * SmU | SOOOOOOOOOOSOOOOO | 748 |
| 6469 | GGCTU | fUmG fGmG fAmG fGmCT * SmU | OOOOS | |
| WV- | TGUCCAGCUUUAUUGGGA | T * S fGmU fCmC fAmG fCmU fUmU fAmU * S | SOOOOOOOOOOOSOOOO | 749 |
| 6470 | GGCTU | fUmG fGmG fAmG fGmCT * SmU | OOOOS | |
| WV- | TGUCCAGCUUUAUUGGGA | T * S fGmU fCmC fAmG fCmU fUmU fAmU fU * | SOOOOOOOOOOOOSOOO | 750 |
| 6496 | GGCTU | SmG fGmG fAmG fGmCT * SmU | OOOOS | |
| WV- | TGUCCAGCUUUAUUGGGA | T * S fGmU fCmC fAmG fCmU fUmU fAmU fUmG | SOOOOOOOOOOOOOSOO | 751 |
| 6497 | GGCTU | * S fGmG fAmG fGmCT * SmU | OOOOS | |
| WV- | TGUCCAGCUUUAUUGGGA | T * S fGmU fCmC fAmG fCmU fUmU fAmU fUmG | SOOOOOOOOOOOOOOSO | 752 |
| 6498 | GGCTU | fG * SmG fAmG fGmCT * SmU | OOOOS | |
| WV- | TGUCCAGCUUUAUUGGGA | T * S fGmU fCmC fAmG fCmU fUmU fAmU fUmG | SOOOOOOOOOOOOOOOS | 753 |
| 6499 | GGCTU | fGmG * S fAmG fGmCT * SmU | OOOOS | |
| WV- | TGUCCAGCUUUAUUGGGA | T * S fGmU fCmC fAmG fCmU fUmU fAmU fUmG | SOOOOOOOOOOOOOOO | 754 |
| 6500 | GGCTU | fGmG fA * SmG fGmCT * SmU | OSOOOS | |
| WV- | TGUCCAGCUUUAUUGGGA | T * S fGmU fCmC fAmG fCmU fUmU fAmU fUmG | SOOOOOOOOOOOOOOO | 755 |
| 6501 | GGCTU | fGmG fAmG * S fGmCT * SmU | OOSOOS | |
| WV- | TGUCCAGCUUUAUUGGGA | T * S fGmU fCmC fAmG fCmU fUmU fAmU fUmG | SOOOOOOOOOOOOOOO | 756 |
| 6502 | GGCTU | fGmG fAmG fG * SmCT * SmU | OOOSOS | |
| WV- | TGUCCAGCUUUAUUGGGA | T * S fGmU fCmC fAmG fCmU fUmU fAmU fUmG | SOOOOOOOOOOOOOOO | 757 |
| 6503 | GGCTU | fGmG fAmG fGmC * ST * SmU | OOOOSS | |
| WV- | TGUCCAGCUUUAUUGGGA | T * S fG * mU fC * mC fA * mG fC * mU * fU * | SXOXOXOXXXOXXXXXXXXX | 758 |
| 6504 | GGCTU | mU fA * mU * fU * mG * fG * mG * fA * mG * fG | XS | |
| | | * mC * AMC6T * SmU | | |
| WV- | TGUCCAGCUUUAUUGGGA | T * S fG * mU fC * mC fA * mG fC * mU * fU * | SXOXOXOXXXOXXXXOOO | 759 |
| 6505 | GGCTU | mU fA * mU * fU * mG * fGmG fAmG fGmC * | OOSS | |
| | | SAMC6T * SmU | | |
| WV- | AGCTTCTTGTCCAGCTTTAT | Mod001L001Aeo * SGeo * Sm5Ceo * STeo * | OSSSSSSSSSSSSSSSSSSS | 760 |
| 6541 | | STeo * SC * ST * ST * SG * ST * SC * SC * SA * SG | | |
| | | * SC * STeo * STeo * STeo * SAeo * STeo | | |
| WV- | AGCTTCTTGTCCAGCTTTAT | Mod001L001Aeo * SGeo * Rm5Ceo * RTeo * | OSRRRRSSSSSSSSSRRRRS | 761 |
| 6542 | | RTeo * RC * ST * ST * SG * ST * SC * SC * SA * SG | | |
| | | * SC * RTeo * RTeo * RTeo * RAeo * STeo | | |
| WV- | AGCTTCTTGTCCAGCTTTAT | Mod001L001Aeo * SGeo * Rm5Ceo * RTeo * | OSRRRSSSSSSSSSSSRRRS | 762 |
| 6543 | | RTeo * SC * ST * ST * SG * ST * SC * SC * SA * SG | | |
| | | * SC * STeo * RTeo * RTeo * RAeo * STeo | | |
| WV- | AGCTTCTTGTCCAGCTTTAT | Mod001L001Aeo * Geom5CeoTeoTeo * C * T * T | OXOOOXXXXXXXXXXXOOO | 763 |
| 6544 | | * G * T * C * C * A * G * C * TeoTeoTeoAeo * Teo | X | |
| WV- | AGCTTCTTGTCCAGCTTTAT | Mod001L001Aeo * SGeom5CeoTeoTeo * RC * ST | OSOOORSSSSSSSSSROOOS | 764 |
| 6545 | | * ST * SG * ST * SC * SC * SA * SG * SC * | | |
| | | RTeoTeoTeoAeo * STeo | | |
| WV- | AGCTTCTTGTCCAGCTTTAT | Mod001L001Aeo * SGeom5CeoTeoTeo * SC * ST | OSOOOSSSSSSSSSSSOOOS | 765 |
| 6546 | | * ST * SG * ST * SC * SC * SA * SG * SC * | | |
| | | STeoTeoTeoAeo * STeo | | |
| WV- | AGCUTCTTGTCCAGCTUUA | Mod001L001mA * SmGmCmUTeo * SC * ST * ST | OSOOOSSSSRSSRSSSOOOS | 766 |
| 6547 | U | * SG * RT * SC * SC * RA * SG * SC * | | |
| | | STeomUmUmA * SmU | | |
| WV- | AGCUUCTTGTCCAGCUUU | Mod001L001mA * RmG * RmC * RmU * RmU * | ORRRRRSSSRSSRSSRRRRR | 767 |
| 6548 | AU | RC * ST * ST * SG * RT * SC * SC * RA * SG * SC * | | |
| | | RmU * RmU * RmU * RmA * RmU | | |
| WV- | AGCUUCTTGTCCAGCUUU | Mod001L001mA * SmG *SmC* SmU *SmU * SC | OSSSSSSSSSSSSSSSSSSS | 768 |
| 6549 | AU | * ST * ST * SG * ST * SC * SC * SA * SG * SC * | | |
| | | SmU * SmU * SmU * SmA * SmU | | |
| WV- | AGCUUCTTGTCCAGCUUU | Mod001L001mA * SmG * RmC * RmU * RmU * | OSRRRRSSSRSSRSSRRRRS | 769 |
| 6550 | AU | RC * ST * ST * SG * RT * SC * SC * RA * SG * SC * | | |
| | | RmU * RmU * RmU * RmA * SmU | | |
| WV- | AGCUUCTTGTCCAGCUUU | Mod001L001mA * SmG * RmC * RmU * RmU * | OSRRRSSSSRSSRSSSRRRS | 770 |
| 6551 | AU | SC * ST * ST * SG * RT * SC * SC * RA * SG * SC * | | |
| | | SmU * RmU * RmU * RmA * SmU | | |
| WV- | AGCUUCTTGTCCAGCUUU | Mod001L001mA * mGmCmUmU * C * T * T * G | OXOOOXXXXXXXXXXXOOO | 771 |
| 6552 | AU | * T * C * C * A * G * C * mUmUmUmA * mU | X | |
| WV- | AGCUUCTTGTCCAGCUUU | Mod001L001mA * SmGmCmUmU * RC * ST * ST | OSOOORSSSRSSRSSROOO | 772 |
| 6553 | AU | * SG * RT * SC * SC * RA * SG * SC * | S | |
| | | RmUmUmUmA * SmU | | |
| WV- | AGCUUCTTGTCCAGCUUU | Mod001L001mA * SmGmCmUmU * SC * ST * ST | OSOOOSSSSRSSRSSSOOOS | 773 |
| 6554 | AU | * SG * RT * SC * SC * RA * SG * SC * | | |
| | | SmUmUmUmA * SmU | | |
| WV- | AGCTTCTTGTCCAGCTTTAT | Mod001L001Aeo * SGeo * Sm5Ceo * STeo * | OSSSSSSSSRSSRSSSSSSS | 774 |
| 6555 | | STeo * SC * ST * ST * SG * RT * SC * SC * RA * SG | | |
| | | * SC * STeo * STeo * STeo * SAeo * STeo | | |
| WV- | AGCTTCTTGTCCAGCTTTAT | Mod001L001Aeo * SGeo * Rm5Ceo * RTeo * | OSRRRRSSSRSSRSSRRRRS | 775 |
| 6556 | | RTeo * RC * ST * ST * SG * RT * SC * SC * RA * SG | | |
| | | * SC * RTeo * RTeo * RTeo * RAeo * STeo | | |
| WV- | AGCTTCTTGTCCAGCTTTAT | Mod001L001Aeo * SGeo * Rm5Ceo * RTeo * | OSRRRSSSSRSSRSSSRRRS | 776 |
| 6557 | | RTeo * SC * ST * ST * SG * RT * SC * SC * RA * SG | | |
| | | * SC * STeo * RTeo * RTeo * RAeo * STeo | | |
| WV- | AGCTTCTTGTCCAGCTTTAT | Mod001L001Aeo * SGeom5CeoTeoTeo * RC * ST | OSOOORSSSRSSRSSROOO | 777 |
| 6558 | | * ST * SG * RT * SC * SC * RA * SG * SC * | S | |
| | | RTeoTeoTeoAeo * STeo | | |
| WV- | AGCTTCTTGTCCAGCTTTAT | Mod001L001Aeo * SGeom5CeoTeoTeo * SC * ST | OSOOOSSSSRSSRSSSOOOS | 778 |
| 6559 | | * ST * SG * RT * SC * SC * RA * SG * SC * | | |
| | | STeoTeoTeoAeo * STeo | | |
| WV- | AGCTTCTTGTCCAGCTTTAT | Mod001L001Aeo * RGeom5CeoTeoTeo * RC * ST | OROOORSSSRSSRSSROOO | 779 |
| 6561 | | * ST * SG * RT * SC * SC * RA * SG * SC * | R | |
| | | RTeoTeoTeoAeo * RTeo | | |
| WV- | AGCTTCTTGTCCAGCTTTAT | Mod001L001Aeo * RGeom5CeoTeoTeo * SC * ST | OROOOSSSSRSSRSSSOOO | 780 |
| 6562 | | * ST * SG * RT * SC * SC * RA * SG * SC * | R | |
| | | STeoTeoTeoAeo * RTeo | | |
| WV- | AGCUTCTTGTCCAGCTUUA | Mod001L001Aeo * mG * mC * mU * Teo * C * T | OXXXXXXXXXXXXXXXXXXX | 781 |
| 6563 | T | * T * G * T * C * C * A * G * C * Teo * mU * mU * | | |
| | | Aeo * Teo | | |
| WV- | AGCUTCTTGTCCAGCTUUA | Mod001L001Aeo * RmG * RmC * RmU * RTeo * | ORRRRRSSSRSSRSSRRRRR | 782 |
| 6564 | T | RC * ST * ST * SG * RT * SC * SC * RA * SG * SC * | | |
| | | RTeo * RmU * RmU * RAeo * RTeo | | |
| WV- | AGCUTCTTGTCCAGCTUUA | Mod001L001Aeo * SmG * SmC * SmU * STeo * | OSSSSSSSSRSSRSSSSSSS | 783 |
| 6565 | T | SC * ST * ST * SG * RT * SC * SC * RA * SG * SC * | | |
| | | STeo * SmU * SmU * SAeo * STeo | | |
| WV- | AGCUUCTTGTCCAGCTUUA | Mod001L001Aeo * SmG * RmC * RmU * RmU * | OSRRRRSSSRSSRSSRRRRS | 784 |
| 6566 | T | RC * ST * ST * SG * RT * SC * SC * RA * SG * SC * | | |
| | | RTeo * RmU * RmU * RAeo * STeo | | |
| WV- | AGCUTCTTGTCCAGCTUUA | Mod001L001Aeo * SmG * RmC * RmU * RTeo * | OSRRRSSSSRSSRSSSRRRS | 785 |
| 6567 | T | SC * ST * ST * SG * RT * SC * SC * RA * SG * SC * | | |
| | | STeo * RmU * RmU * RAeo * STeo | | |
| WV- | AGCUTCTTGTCCAGCTUUA | Mod001L001Aeo * mGmCmUTeo * C * T * T * G | OXOOOXXXXXXXXXXXOOO | 786 |
| 6568 | T | * T * C * C * A * G * C * TeomUmUAeo * Teo | X | |
| WV- | AGCUTCTTGTCCAGCTUUA | Mod001L001Aeo * SmGmCmUTeo * RC * ST * ST | OSOOORSSSRSSRSSROOO | 787 |
| 6569 | T | * SG * RT * SC * SC * RA * SG * SC * | S | |
| | | RTeomUmUAeo * STeo | | |
| WV- | AGCUTCTTGTCCAGCTUUA | Mod001L001Aeo * SmGmCmUTeo * SC * ST * ST | OSOOOSSSSRSSRSSSOOOS | 788 |
| 6570 | T | * SG * RT * SC * SC * RA * SG * SC * | | |
| | | STeomUmUAeo * STeo | | |
| WV- | AGCUTCTTGTCCAGCTUUA | Mod001L001Aeo * RmGmCmUTeo * RC * ST * | OROOORSSSRSSRSSROOO | 789 |
| 6571 | T | ST * SG * RT * SC * SC * RA * SG * SC * | R | |
| | | RTeomUmUAeo * RTeo | | |
| WV- | AGCUTCTTGTCCAGCTUUA | Mod001L001Aeo * RmGmCmUTeo * SC * ST * ST | OROOOSSSSRSSRSSSOOO | 790 |
| 6572 | T | * SG * RT * SC * SC * RA * SG * SC * | R | |
| | | STeomUmUAeo * RTeo | | |
| WV- | AGCTTCTTGTCCAGCTTTAT | Mod001L001Aeo * SGeo * Sm5Ceo * STeo * | OSSSSRSSSRSSRSSRSSSS | 791 |
| 6757 | | STeo * RC * ST * ST * SG * RT * SC * SC * RA * SG | | |
| | | * SC * RTeo * STeo * STeo * SAeo * STeo | | |
| WV- | AGCTTCTTGTCCAGCTTTAT | Aeo * SGeo * Sm5Ceo * STeo * STeo * SC * ST * | SSSSSSSSRSSRSSSSSSS | 792 |
| 6758 | | ST * SG * RT * SC * SC * RA * SG * SC * STeo * | | |
| | | STeo * STeo * SAeo * STeo | | |
| WV- | AGCTTCTTGTCCAGCTTTAT | Aeo * SGeo * Sm5Ceo * STeo * STeo * RC * ST * | SSSSRSSSRSSRSSRSSSS | 793 |
| 6759 | | ST * SG * RT * SC * SC * RA * SG * SC * RTeo * | | |
| | | STeo * STeo * SAeo * STeo | | |
| WV- | AGCTTCTTGTCCAGCTTTAT | Aeo * Geom5CeoTeoTeo * C * T * T * G * T * C * | XOOOXXXXXXXXXXXOOOX | 794 |
| 6760 | | C * A * G * C * TeoTeoTeoAeo * Teo | | |
| WV- | AGCTTCTTGTCCAGCTTTAT | Aeo * SGeom5CeoTeoTeo * RC * ST * ST * SG * | SOOORSSSRSSRSSROOOS | 795 |
| 6761 | | RT * SC * SC * RA * SG * SC * RTeoTeoTeoAeo * | | |
| | | STeo | | |
| WV- | AGCTTCTTGTCCAGCTTTAT | Aeo * SGeom5CeoTeoTeo * SC * ST * ST * SG * | SOOOSSSSRSSRSSSOOOS | 796 |
| 6762 | | RT * SC * SC * RA * SG * SC * STeoTeoTeoAeo * | | |
| | | STeo | | |
| WV- | TGUCCAGCUUUAUUGGGA | T * S fG * SmU fC * SmC fA * SmG fC * SmU fU * | SSOSOSOSOSOSOSSSSSSSS | 797 |
| 6763 | GGCTU | SmU fA * SmU fU * SmG * S fG * SmG * S fA * | S | |
| | | SmG * S fG * SmC * ST * SmU | | |
| WV- | TAGCUUCUUGUCCAGCUU | T * S fA * SmG fC * SmU * S fU * SmC * S fU * | SSOSSSSSSSOSOSSSSSSSSS | 798 |
| 6764 | UAUTU | SmU * S fG * SmU fC * SmC fA * SmG * S fC * | | |
| | | SmU * S fU * SmU * S fA * SmU * ST * SmU | | |
| WV- | TAGCUUCUUGUCCAGCUU | T * S fA * SmG fC * SmU fU * SmC fU * SmU fG * | SSOSOSOSOSOSOSSSSSSSS | 799 |
| 6765 | UAUTU | SmU fC * SmC fA * SmG * S fC * SmU * S fU * | S | |
| | | SmU * S fA * SmU * ST * SmU | | |
| WV- | TGUCCAGCUUUAUUGGGA | VPT * fG * mU fC * mC fA * mG fC * mU fU * mU | XXOXOXOXOXOXOXOOOO | 800 |
| 6766 | GTU | fA * mU fU * mG fGmG fAmG * T * mU | XX | |
| WV- | AGCTTCTTGTCCAGCTTTAT | L001Aeo * SGeo * Sm5Ceo * STeo * STeo * SC * | OSSSSSSSSRSSRSSSSSSS | 801 |
| 7104 | | ST * ST * SG * RT * SC * SC * RA * SG * SC * STeo | | |
| | | * STeo * STeo * SAeo * STeo | | |
| WV- | AGCTTCTTGTCCAGCTTTAT | L001Aeo * SGeo * Sm5Ceo * STeo * STeo * RC * | OSSSSRSSSRSSRSSRSSSS | 802 |
| 7105 | | ST * ST * SG * RT * SC * SC * RA * SG * SC * RTeo | | |
| | | * STeo * STeo * SAeo * STeo | | |
| WV- | AGCTTCTTGTCCAGCTTTAT | L001Aeo * Geom5CeoTeoTeo * C * T * T * G * T | OXOOOXXXXXXXXXXXOOO | 803 |
| 7106 | | * C * C * A * G * C * TeoTeoTeoAeo * Teo | X | |
| WV- | AGCTTCTTGTCCAGCTTTAT | L001Aeo * SGeom5CeoTeoTeo * RC * ST * ST * | OSOOORSSSRSSRSSROOO | 804 |
| 7107 | | SG * RT * SC * SC * RA * SG * SC * | S | |
| | | RTeoTeoTeoAeo * STeo | | |
| WV- | AGCTTCTTGTCCAGCTTTAT | L001Aeo * SGeom5CeoTeoTeo * SC * ST * ST * | OSOOOSSSSRSSRSSSOOOS | 805 |
| 7108 | | SG * RT * SC * SC * RA * SG * SC * | | |
| | | STeoTeoTeoAeo * STeo | | |
| WV- | ATAGCAGCTTCTTGTCCAG | Aeo * Teo * Aeo * Geo * m5Ceo * A * G * C * T * | XXXXXXXXXXXXXXXXXXX | 806 |
| 7139 | C | T * C * T * T * G * T * m5Ceo * m5Ceo * Aeo * | | |
| | | Geo * m5Ceo | | |
| WV- | ATAGCAGCTTCTTGTCCAG | mA * Teo * Aeo * Geo * m5Ceo * A * G * C * T * | XXXXXXXXXXXXXXXXXXX | 807 |
| 7140 | C | T * C * T * T * G * T * m5Ceo * m5Ceo * Aeo * | | |
| | | Geo * mC | | |
| WV- | AUAGCAGCTTCTTGTCCAU | Aeo * mU * Aeo * Geo * m5Ceo * A * G * C * T * | XXXXXXXXXXXXXXXXXXX | 808 |
| 7141 | C | T * C * T * T * G * T * m5Ceo * m5Ceo * Aeo * | | |
| | | mU * m5Ceo | | |
| WV- | ATAGCAGCTTCTTGTCCAG | Aeo * Teo * mA * Geo * m5Ceo * A * G * C * T * | XXXXXXXXXXXXXXXXXXX | 809 |
| 7142 | C | T * C * T * T * G * T * m5Ceo * m5Ceo * mA * | | |
| | | Geo * m5Ceo | | |
| WV- | ATAGCAGCTTCTTGTCCAG | Aeo * Teo * Aeo * mG * m5Ceo * A * G * C * T * | XXXXXXXXXXXXXXXXXXX | 810 |
| 7143 | C | T * C * T * T * G * T * m5Ceo * mC * Aeo * Geo * | | |
| | | m5Ceo | | |
| WV- | ATAGCAGCTTCTTGTCCAG | Aeo * Teo * Aeo * Geo * mC * A * G * C * T * T * | XXXXXXXXXXXXXXXXXXX | 811 |
| 7144 | C | C * T * T * G * T * mC * m5Ceo * Aeo * Geo * | | |
| | | m5Ceo | | |
| WV- | AUAGCAGCTTCTTGTCCAG | mA * mU * Aeo * Geo * m5Ceo * A * G * C * T * | XXXXXXXXXXXXXXXXXXX | 812 |
| 7145 | C | T * C * T * T * G * T * m5Ceo * m5Ceo * Aeo * | | |
| | | mG * mC | | |
| WV- | ATAGCAGCTTCTTGTCCAG | mA * Teo * mA * Geo * m5Ceo * A * G * C * T * | XXXXXXXXXXXXXXXXXXX | 813 |
| 7146 | C | T * C * T * T * G * T * m5Ceo * m5Ceo * mA * | | |
| | | Geo * mC | | |
| WV- | ATAGCAGCTTCTTGTCCAG | mA * Teo * Aeo * mG * m5Ceo * A * G * C * T * | XXXXXXXXXXXXXXXXXXX | 814 |
| 7147 | C | T * C * T * T * G * T * m5Ceo * mC * Aeo * Geo * | | |
| | | mC | | |
| WV- | ATAGCAGCTTCTTGTCCAG | mA * Teo * Aeo * Geo * mC * A * G * C * T * T * | XXXXXXXXXXXXXXXXXXX | 815 |
| 7148 | C | C * T * T * G * T * mC * m5Ceo * Aeo * Geo * mC | | |
| WV- | AUAGCAGCTTCTTGTCCAU | Aeo * mU * mA * Geo * m5Ceo * A * G * C * T * | XXXXXXXXXXXXXXXXXXX | 816 |
| 7149 | C | T * C * T * T * G * T * m5Ceo * m5Ceo * mA * | | |
| | | mU * m5Ceo | | |
| WV- | AUAGCAGCTTCTTGTCCAU | Aeo * mU * Aeo * mG * m5Ceo * A * G * C * T * | XXXXXXXXXXXXXXXXXXX | 817 |
| 7150 | C | T * C * T * T * G * T * m5Ceo * mC * Aeo * mU * | | |
| | | m5Ceo | | |
| WV- | AUAGCAGCTTCTTGTCCAU | Aeo * mU * Aeo * Geo * mC * A * G * C * T * T * | XXXXXXXXXXXXXXXXXXX | 818 |
| 7151 | C | C * T * T * G * T * mC * m5Ceo * Aeo * mU * | | |
| | | m5Ceo | | |
| WV- | ATAGCAGCTTCTTGTCCAG | Aeo * Teo * mA * mG * m5Ceo * A * G * C * T * | XXXXXXXXXXXXXXXXXXX | 819 |
| 7152 | C | T * C * T * T * G * T * m5Ceo * mC * mA * Geo * | | |
| | | m5Ceo | | |
| WV- | ATAGCAGCTTCTTGTCCAG | Aeo * Teo * mA * Geo * mC * A * G * C * T * T * | XXXXXXXXXXXXXXXXXXX | 820 |
| 7153 | C | C * T * T * G * T * mC * m5Ceo * mA * Geo * | | |
| | | m5Ceo | | |
| WV- | ATAGCAGCTTCTTGTCCAG | Aeo * Teo * Aeo * mG * mC * A * G * C * T * T * | XXXXXXXXXXXXXXXXXXX | 821 |
| 7154 | C | C * T * T * G * T * mC * mC * Aeo * Geo * m5Ceo | | |
| WV- | AUAGCAGCTTCTTGTCCAG | mA * mU * mA * Geo * m5Ceo * A * G * C * T * | XXXXXXXXXXXXXXXXXXX | 822 |
| 7155 | C | T * C * T * T * G * T * m5Ceo * m5Ceo * mA * | | |
| | | mG * mC | | |
| WV- | AUAGCAGCTTCTTGTCCAG | mA * mU * Aeo * mG * m5Ceo * A * G * C * T * | XXXXXXXXXXXXXXXXXXX | 823 |
| 7156 | C | T * C * T * T * G * T * m5Ceo * mC * Aeo * mG * | | |
| | | mC | | |
| WV- | AUAGCAGCTTCTTGTCCAG | mA * mU * Aeo * Geo * mC * A * G * C * T * T * | XXXXXXXXXXXXXXXXXXX | 824 |
| 7157 | C | C * T * T * G * T * mC * m5Ceo * Aeo * mG * mC | | |
| WV- | ATAGCAGCTTCTTGTCGAG | mA * Teo * mA * mG * m5Ceo * A * G * C * T * T | XXXXXXXXXXXXXXXXXXX | 825 |
| 7158 | C | * C * T * T * G * T * m5Ceo * mG * mA * Geo * | | |
| | | mC | | |
| WV- | ATAGCAGCTTCTTGTCCAG | mA * Teo * mA * Geo * mC * A * G * C * T * T * | XXXXXXXXXXXXXXXXXXX | 826 |
| 7159 | C | C * T * T * G * T * mC * m5Ceo * mA * Geo * mC | | |
| WV- | ATAGCAGCTTCTTGTCCAG | mA * Teo * Aeo * mG * mC * A * G * C * T * T * | XXXXXXXXXXXXXXXXXXX | 827 |
| 7160 | C | C * T * T * G * T * mC * mC * Aeo * Geo * mC | | |
| WV- | AUAGCAGCTTCTTGTCCAU | Aeo * mU * mA * mG * m5Ceo * A * G * C * T * | XXXXXXXXXXXXXXXXXXX | 828 |
| 7161 | C | T * C * T * T * G * T * m5Ceo * mC * mA * mU * | | |
| | | m5Ceo | | |
| WV- | AUAGCAGCTTCTTGTCCAU | Aeo * mU * mA * Geo * mC * A * G * C * T * T * | XXXXXXXXXXXXXXXXXXX | 829 |
| 7162 | C | C * T * T * G * T * mC * m5Ceo * mA * mU * | | |
| | | m5Ceo | | |
| WV- | AUAGCAGCTTCTTGTCCAU | Aeo * mU * Aeo * mG * mC * A * G * C * T * T * | XXXXXXXXXXXXXXXXXXX | 830 |
| 7163 | C | C * T * T * G * T * mC * mC * Aeo * mU * m5Ceo | | |
| WV- | ATAGCAGCTTCTTGTCCAG | Aeo * Teo * mA * mG * mC * A * G * C * T * T * | XXXXXXXXXXXXXXXXXXX | 831 |
| 7164 | C | C * T * T * G * T * mC * mC * mA * Geo * m5Ceo | | |
| WV- | AUAGCAGCTTCTTGTCCAG | mA * mU * mA * mG * m5Ceo * A * G * C * T * T | XXXXXXXXXXXXXXXXXXX | 832 |
| 7165 | C | * C * T * T * G * T * m5Ceo * mC * mA * mG * | | |
| | | mC | | |
| WV- | AUAGCAGCTTCTTGTCCAG | mA * mU * mA * Geo * mC * A * G * C * T * T * C | XXXXXXXXXXXXXXXXXXX | 833 |
| 7166 | C | * T * T * G * T * mC * m5Ceo * mA * mG * mC | | |
| WV- | AUAGCAGCTTCTTGTCCAG | mA * mU * Aeo * mG * mC * A * G * C * T * T * C | XXXXXXXXXXXXXXXXXXX | 834 |
| 7167 | C | * T * T * G * T * mC * mC * Aeo * mG * mC | | |
| WV- | ATAGCAGCTTCTTGTCCAG | mA * Teo * mA * mG * mC * A * G * C * T * T * C | XXXXXXXXXXXXXXXXXXX | 835 |
| 7168 | C | * T * T * G * T * mC * mC * mA * Geo * mC | | |
| WV- | AUAGCAGCTTCTTGTCCAG | Aeo * mU * mA * mG * mC * A * G * C * T * T * C | XXXXXXXXXXXXXXXXXXX | 836 |
| 7169 | C | * T * T * G * T * mC * mC * mA * mG * m5Ceo | | |
| WV- | AUAGCAGCTTCTTGTCCAG | Aeo * mU * Aeo * Geo * m5Ceo * A * G * C * T * | XXXXXXXXXXXXXXXXXXX | 837 |
| 7170 | C | T * C * T * T * G * T * m5Ceo * m5Ceo * Aeo * | | |
| | | mG * m5Ceo | | |
| WV- | AUAGCAGCTTCTTGTCCAG | Aeo * mU * mA * Geo * m5Ceo * A * G * C * T * | XXXXXXXXXXXXXXXXXXX | 838 |
| 7171 | C | T * C * T * T * G * T * m5Ceo * m5Ceo * mA * | | |
| | | mG * m5Ceo | | |
| WV- | AUAGCAGCTTCTTGTCCAG | Aeo * mU * Aeo * mG * m5Ceo * A * G * C * T * | XXXXXXXXXXXXXXXXXXX | 839 |
| 7172 | C | T * C * T * T * G * T * m5Ceo * mC * Aeo * mG * | | |
| | | m5Ceo | | |
| WV- | AUAGCAGCTTCTTGTCCAG | Aeo * mU * Aeo * Geo * mC * A * G * C * T * T * | XXXXXXXXXXXXXXXXXXX | 840 |
| 7173 | C | C * T * T * G * T * mC * m5Ceo * Aeo * mG * | | |
| | | m5Ceo | | |
| WV- | AUAGCAGCTTCTTGTCCAG | Aeo * mU * mA * mG * m5Ceo * A * G * C * T * | XXXXXXXXXXXXXXXXXXX | 841 |
| 7174 | C | T * C * T * T * G * T * m5Ceo * mC * mA * mG * | | |
| | | m5Ceo | | |
| WV- | AUAGCAGCTTCTTGTCCAG | Aeo * mU * mA * Geo * mC * A * G * C * T * T * | XXXXXXXXXXXXXXXXXXX | 842 |
| 7175 | C | C * T * T * G * T * mC * m5Ceo * mA * mG * | | |
| | | m5Ceo | | |
| WV- | AUAGCAGCTTCTTGTCCAG | Aeo * mU * Aeo * mG * mC * A * G * C * T * T * | XXXXXXXXXXXXXXXXXXX | 843 |
| 7176 | C | C * T * T * G * T * mC * mC * Aeo * mG * m5Ceo | | |
| WV- | TGUCCAGCUUAUUGGGAG | VPT * fG * mU fC * mC fA * mG fC * ImU fU * mU | XXOXOXOXOXOXOXXXXXX | 844 |
| 7302 | GCTU | fA * mU fU * mG * fG * mG * fA * mG * fG * mC | XXS | |
| | | * TGaNC6T * SmU | | |
| WV- | TGUCCAGCUUAUUGGGAG | VPT * fG * mU fC * mC fA * mG fC * mU fU * ImU | XXOXOXOXOXOXOXXXXXX | 845 |
| 7303 | GCTU | fA * mU fU * mG * fG * mG * fA * mG * fG * mC | XXS | |
| | | * TGaNC6T * SmU | | |
| WV- | TGUCCAGCUUAUUGGGAG | VPT * fG * mU fC * mC fA * mG fC * ImU * fU * | XXOXOXOXXXOXXXXXXXX | 846 |
| 7304 | GCTU | mU fA * mU * fU * mG * fG * mG * fA * mG * fG | XXS | |
| | | * mC * TGaNC6T * SmU | | |
| WV- | TGUCCAGCUUAUUGGGAG | VPT * fG * mU fC * mC fA * mG fC * mU * fU * | XXOXOXOXXXOXXXXXXXX | 847 |
| 7305 | GCTU | ImU fA * mU * fU * mG * fG * mG * fA * mG * fG | XXS | |
| | | * mC * TGaNC6T * SmU | | |
| WV- | TGUCCAGCUUAUUGGGAG | VPT * fG * mU fC * mC fA * mG fC * ImU fU * mU | XXOXOXOXOXOXOXXXXXX | 848 |
| 7323 | GCTU | fA * mU fU * mG * fG * mG * fA * mG * fG * mC | XXS | |
| | | * AMC6T * SmU | | |
| WV- | TGUCCAGCUUAUUGGGAG | VPT * fG * mU fC * mC fA * mG fC * mU fU * ImU | XXOXOXOXOXOXOXXXXXX | 849 |
| 7324 | GCTU | fA * mU fU * mG * fG * mG * fA * mG * fG * mC | XXS | |
| | | * AMC6T * SmU | | |
| WV- | TGUCCAGCUUAUUGGGAG | VPT * fG * mU fC * mC fA * mG fC * ImU * fU * | XXOXOXOXXXOXXXXXXXX | 850 |
| 7325 | GCTU | mU fA * mU * fU * mG * fG * mG * fA * mG * fG | XXS | |
| | | * mC * AMC6T * SmU | | |
| WV- | TGUCCAGCUUAUUGGGAG | VPT * fG * mU fC * mC fA * mG fC * mU * fU * | XXOXOXOXXXOXXXXXXXX | 851 |
| 7326 | GCTU | ImU fA * mU * fU * mG * fG * mG * fA * mG * fG | XXS | |
| | | * mC * AMC6T * SmU | | |
| WV- | TGUCCAGCUUUAUUGGGA | T * S fG * SmU fC * SmC fA * SmG fC * SmU * S | SSOSOSOSSSOSSSSSSSSSSS | 852 |
| 7490 | GGCTU | fU * SmU fA * SmU * S fU * SmG * S fG * SmG * | | |
| | | S fA * SmG * S fG * SmC * STGaNC6T * SmU | | |
| WV- | TAGCUUCUUGUCCAGCUU | T * S fA * SmG fC * SmU * S fU * SmC * S fU * | SSOSSSSSSSOSOSSSSSSSSS | 853 |
| 7491 | UAUTU | SmU * S fG * SmU fC * SmC fA * SmG * S fC * | | |
| | | SmU * S fU * SmU * S fA * SmU * STGaNC6T * | | |
| | | SmU | | |
| WV- | TGUCCAGCUUUAUUGGGA | VPT * S fG * SmU fC * SmC fA * SmG fC * SmU * | SSOSOSOSSSOSSSSSSSSSSS | 854 |
| 7492 | GGCTU | S fU * SmU fA * SmU * S fU * SmG * S fG * SmG | | |
| | | * S fA * SmG * S fG * SmC * STGaNC6T * SmU | | |
| WV- | TAGCUUCUUGUCCAGCUU | VPT * S fA * SmG fC * SmU * S fU * SmC * S fU * | SSOSSSSSSSOSOSSSSSSSSS | 855 |
| 7493 | UAUTU | SmU * S fG * SmU fC * SmC fA * SmG * S fC * | | |
| | | SmU * S fU * SmU * S fA * SmU * STGaNC6T * | | |
| | | SmU | | |
| WV- | TGUCCAGCUUUAUUGGGA | T * S fG * SmU fC * SmC fA * SmG fC * SImU * S | SSOSOSOSSSOSSSSSSSSSSS | 856 |
| 7494 | GGCTU | fU * SmU fA * SmU * S fU * SmG * S fG * SmG * | | |
| | | S fA * SmG * S fG * SmC * STGaNC6T * SmU | | |
| WV- | TAGCUUCUUGUCCAGCUU | T * S fA * SmG fC * SmU * S fU * SmC * S fU * | SSOSSSSSSSOSOSSSSSSSSS | 857 |
| 7495 | UAUTU | SImU * S fG * SmU fC * SmC fA * SmG * S fC * | | |
| | | SmU * S fU * SmU * S fA * SmU * STGaNC6T * | | |
| | | SmU | | |
| WV- | TGUCCAGCUUUAUUGGGA | VPT * S fG * SmU fC * SmC fA * SmG fC * SImU * | SSOSOSOSSSOSSSSSSSSSSS | 858 |
| 7496 | GGCTU | S fU * SmU fA * SmU * S fU * SmG * S fG * SmG | | |
| | | * S fA * SmG * S fG * SmC * STGaNC6T * SmU | | |
| WV- | TAGCUUCUUGUCCAGCUU | VPT * S fA * SmG fC * SmU * S fU * SmC * S fU * | SSOSSSSSSSOSOSSSSSSSSS | 859 |
| 7497 | UAUTU | SImU * S fG * SmU fC * SmC fA * SmG * S fC * | | |
| | | SmU * S fU * SmU * S fA * SmU * STGaNC6T * | | |
| | | SmU | | |
| WV- | TGUCCAGCUUUAUUGGGA | T * S fG * SmU fC * SmC fA * SmG fC * SmU * S | SSOSOSOSSSOSSSSSSSSSSS | 860 |
| 7498 | GGCTU | fU * SmU fA * SmU * S fU * SmG * S fG * SmG * | | |
| | | S fA * SmG * S fG * SmC * SAMC6T * SmU | | |
| WV- | TAGCUUCUUGUCCAGCUU | T * S fA * SmG fC * SmU * S fU * SmC * S fU * | SSOSSSSSSSOSOSSSSSSSSS | 861 |
| 7499 | UAUTU | SmU * S fG * SmU fC * SmC fA * SmG * S fC * | | |
| | | SmU * S fU * SmU * S fA * SmU * SAMC6T * SmU | | |
| WV- | TGUCCAGCUUUAUUGGGA | VPT * S fG * SmU fC * SmC fA * SmG fC * SmU * | SSOSOSOSSSOSSSSSSSSSSS | 862 |
| 7500 | GGCTU | S fU * SmU fA * SmU * S fU * SmG * S fG * SmG | | |
| | | * S fA * SmG * S fG * SmC * SAMC6T * SmU | | |
| WV- | TAGCUUCUUGUCCAGCUU | VPT * S fA * SmG fC * SmU * S fU * SmC * S fU * SSOSSSSSSSOSOSSSSSSSSS | 863 | |
| 7501 | UAUTU | SmU * S fG * SmU fC * SmC fA * SmG * S fC * | | |
| | | SmU * S fU * SmU * S fA * SmU * SAMC6T * SmU | | |
| WV- | TGUCCAGCUUUAUUGGGA | T * S fG * SmU fC * SmC fA * SmG fC * SImU * S | SSOSOSOSSSOSSSSSSSSSSS | 864 |
| 7502 | GGCTU | fU * SmU fA * SmU * S fU * SmG * S fG * SmG * | | |
| | | S fA * SmG * S fG * SmC * SAMC6T * SmU | | |
| WV- | TAGCUUCUUGUCCAGCUU | T * S fA * SmG fC * SmU * S fU * SmC * S fU * | SSOSSSSSSSOSOSSSSSSSSS | 865 |
| 7503 | UAUTU | SImU * S fG * SmU fC * SmC fA * SmG * S fC * | | |
| | | SmU * S fU * SmU * S fA * SmU * SAMC6T * SmU | | |
| WV- | TGUCCAGCUUUAUUGGGA | VPT * S fG * SmU fC * SmC fA * SmG fC * SImU * | SSOSOSOSSSOSSSSSSSSSSS | 866 |
| 7504 | GGCTU | S fU * SmU fA * SmU * S fU * SmG * S fG * SmG | | |
| | | * S fA * SmG * S fG * SmC * SAMC6T * SmU | | |
| WV- | TAGCUUCUUGUCCAGCUU | VPT * S fA * SmG fC * SmU * S fU * SmC * S fU * | SSOSSSSSSSOSOSSSSSSSSS | 867 |
| 7505 | UAUTU | SImU * S fG * SmU fC * SmC fA * SmG * S fC * | | |
| | | SmU * S fU * SmU * S fA * SmU * SAMC6T * SmU | | |
| WV- | TGUCCAGCUUUAUUGGGA | T * S fG * SmU * S fC * SmC * S fA * SmG * S fC * | SSSSSSSSSSSSSSSSSSSSSS | 868 |
| 7521 | GGCTU | SmU * S fU * SmU * S fA * SmU * S fU * SmG * S | | |
| | | fG * SmG * S fA * SmG * S fG * SmC * ST * SmU | | |
| WV- | TAGCUUCUUGUCCAGCUU | T * S fA * SmG * S fC * SmU * S fU * SmC * S fU * | SSSSSSSSSSSSSSSSSSSSSS | 869 |
| 7522 | UAUTU | SmU * S fG * SmU * S fC * SmC * S fA * SmG * S | | |
| | | fC * SmU * S fU * SmU * S fA * SmU * ST * SmU | | |
| WV- | TGUCCAGCUUUAUUGGGA | T * S fG * SmU fC * SmC fA * SmG fC * SmU fU * | SSOSOSOSOSOSOSSSSSSSS | 870 |
| 7523 | GGCTU | SmU fA * SmU fU * SG * SG * SG * SA * SG * SG * | S | |
| | | SC * ST * SmU | | |
| WV- | TAGCUUCUUGUCCAGCTTT | T * S fA * SmG fC * SmU fU * SmC fU * SmU fG * | SSOSOSOSOSOSOSSSSSSSS | 871 |
| 7524 | ATTU | SmU fC * SmC fA * SG * SC * ST * ST * ST * SA * | S | |
| | | ST * ST * SmU | | |
| WV- | TGUCCAGCUUUAUUGGGA | T * S fG * SmU fC * SmC fA * SmG fC * SmU * S | SSOSOSOSSSOSSSSSSSSSSS | 872 |
| 7525 | GGCTU | fU * SmU fA * SmU * S fU * SG * SG * SG * SA * | | |
| | | SG * SG * SC * ST * SmU | | |
| WV- | TAGCUUCUUGUCCAGCTTT | T * S fA * SmG fC * SmU * S fU * SmC * S fU * | SSOSSSSSSSOSOSSSSSSSSS | 873 |
| 7526 | ATTU | SmU * S fG * SmU fC * SmC fA * SG * SC * ST * | | |
| | | ST * ST * SA * ST * ST * SmU | | |
| WV- | TGUCCAGCUUUAUUGGGA | T * S fG * SmU * S fC * SmC * S fA * SmG * S fC * | SSSSSSSSSSSSSSSSSSSSSS | 874 |
| 7527 | GGCTU | SmU * S fU * SmU * S fA * SmU * S fU * SG * SG | | |
| | | * SG * SA * SG * SG * SC * ST * SmU | | |
| WV- | TAGCUUCUUGUCCAGCTTT | T * S fA * SmG * S fC * SmU * S fU * SmC * S fU * | SSSSSSSSSSSSSSSSSSSSSS | 875 |
| 7528 | ATTU | SmU * S fG * SmU * S fC * SmC * S fA * SG * SC * | | |
| | | ST * ST * ST * SA * ST * ST * SmU | | |
| WV- | TGUCCAGCUUUAUUGGGA | T * fG * mUmC * mCmA * mGmC * mUmU * | XXOXOXOXOXOXOXXXXXX | 876 |
| 7540 | GGCTU | mUmA * mU fU * mG * mG * mG * mA * mG * | XXX | |
| | | mG * mC * T * mU | | |
| WV- | TGUCCAGCUUUAUUGGGA | T * mG * mUmC * mCmA * mGmC * mUmU * | XXOXOXOXOXOXOXXXXXX | 877 |
| 7541 | GGCTU | mUmA * mUmU * mG * mG * mG * mA * mG * | XXX | |
| | | mG * mC * T * mU | | |
| WV- | TAGCUUCUUGUCCAGCUU | T * fA * mG fC * mU fU * mC fU * mU fG * mU fC | XXOXOXOXOXOXOXXXXXX | 878 |
| 7542 | UAUTU | * mC fA * mG * fC * mU * fU * mU * fA * mU * T | XXX | |
| | | * mU | | |
| WV- | TAGCUUCUUGUCCAGCUU | T * fA * mGmC * mUmU * mCmU * mUmG * | XXOXOXOXOXOXOXXXXXX | 879 |
| 7543 | UAUTU | mUmC * mC fA * mG * mC * mU * mU * mU * | XXX | |
| | | mA * mU * T * mU | | |
| WV- | TAGCUUCUUGUCCAGCUU | T * mA * mGmC * mUmU * mCmU * mUmG * | XXOXOXOXOXOXOXXXXXX | 880 |
| 7544 | UAUTU | mUmC * mCmA * mG * mC * mU * mU * mU * | XXX | |
| | | mA * mU * T * mU | | |
| WV- | TGUCCAGCUUUAUUGGGA | T * S fGmU * S fCmC * S fAmG * S fCmU * S | SOSOSOSOSOSOSOSOSOS | 881 |
| 7597 | GGCTU | fUmU * S fAmU * S fUmG * S fGmG * S fAmG * S | OSS | |
| | | fGmC * ST * SmU | | |
| WV- | TGUCCAGCUUUAUUGGGA | T * S fG * SmU fC * SmC fA * SmG fC * SmU fU * | SSOSOSOSOSOSOSOSOSO | 882 |
| 7598 | GGCTU | SmU fA * SmU fU * SmG fG * SmG fA * SmG fG * | SOS | |
| | | SmCT * SmU | | |
| WV- | TAGCUUCUUGUCCAGCUU | T * S fAmG * S fCmU * S fUmC * S fUmU * S | SOSOSOSOSOSOSOSOSOS | 883 |
| 7599 | UAUTU | fGmU * S fCmC * S fAmG * S fCmU * S fUmU * S | OSS | |
| | | fAmU * ST * SmU | | |
| WV- | TAGCUUCUUGUCCAGCUU | T * S fA * SmG fC * SmU fU * SmC fU * SmU fG * | SSOSOSOSOSOSOSOSOSO | 884 |
| 7600 | UAUTU | SmU fC * SmC fA * SmG fC * SmU fU * SmU fA * | SOS | |
| | | SmUT * SmU | | |
| WV- | TGUCCAGCUUUAUUGGGA | 5MSdT * fG * mU fC * mC fA * mG fC * mU fU * | XXOXOXOXOXOXOXXXXXX | 885 |
| 7635 | GGCTU | mU fA * mU fU * mG * fG * mG * fA * mG * fG * | XXX | |
| | | mC * T * mU | | |
| WV- | TAGCUUCUUGUCCAGCUU | 5MSdT * fA * mG fC * mU fU * mC fU * mU fG * | XXOXOXOXOXOXOXXXXXX | 886 |
| 7636 | UAUTU | mU fC * mC fA * mG * fC * mU * fU * mU * fA * | XXX | |
| | | mU * T * mU | | |
| WV- | TGUCCAGCUUUAUUGGGA | PO5MSdT * fG * mU fC * mC fA * mG fC * mU fU | XXOXOXOXOXOXOXXXXXX | 887 |
| 7637 | GGCTU | * mU fA * mU fU * mG * fG * mG * fA * mG * fG | XXX | |
| | | * mC * T * mU | | |
| WV- | TAGCUUCUUGUCCAGCUU | PO5MSdT * fA * mG fC * mU fU * mC fU * mU fG | XXOXOXOXOXOXOXXXXXX | 888 |
| 7638 | UAUTU | * mU fC * mC fA * mG * fC * mU * fU * mU * fA | XXX | |
| | | * mU * T * mU | | |
| WV- | TGUCCAGCUUUAUUGGGA | PS5MSdT * fG * mU fC * mC fA * mG fC * mU fU | XXOXOXOXOXOXOXXXXXX | 889 |
| 7639 | GGCTU | * mU fA * mU fU * mG * fG * mG * fA * mG * fG | XXX | |
| | | * mC * T * mU | | |
| WV- | TAGCUUCUUGUCCAGCUU | PS5MSdT * fA * mG fC * mU fU * mC fU * mU fG | XXOXOXOXOXOXOXXXXXX | 890 |
| 7640 | UAUTU | * mU fC * mC fA * mG * fC * mU * fU * mU * fA | XXX | |
| | | * mU * T * mU | | |
| WV- | TGUCCAGCUUUAUUGGGA | PH5MSdT * fG * mU fC * mC fA * mG fC * mU fU | XXOXOXOXOXOXOXXXXXX | 891 |
| 7641 | GGCTU | * mU fA * mU fU * mG * fG * mG * fA * mG * fG | XXX | |
| | | * mC * T * mU | | |
| WV- | TAGCUUCUUGUCCAGCUU | PH5MSdT * fA * mG fC * mU fU * mC fU * mU fG | XXOXOXOXOXOXOXXXXXX | 892 |
| 7642 | UAUTU | * mU fC * mC fA * mG * fC * mU * fU * mU * fA | XXX | |
| | | * mU * T * mU | | |
| WV- | TGUCCAGCUUUAUUGGGA | 5MRdT * fG * mU fC * mC fA * mG fC * mU fU * | XXOXOXOXOXOXOXXXXXX | 893 |
| 7643 | GGCTU | mU fA * mU fU * mG * fG * mG * fA * mG * fG * | XXX | |
| | | mC * T * mU | | |
| WV- | TAGCUUCUUGUCCAGCUU | 5MRdT * fA * mG fC * mU fU * mC fU * mU fG * | XXOXOXOXOXOXOXXXXXX | 894 |
| 7644 | UAUTU | mU fC * mC fA * mG * fC * mU * fU * mU * fA * | XXX | |
| | | mU * T * mU | | |
| WV- | TGUCCAGCUUUAUUGGGA | PO5MRdT * fG * mU fC * mC fA * mG fC * mU fU | XXOXOXOXOXOXOXXXXXX | 895 |
| 7645 | GGCTU | * mU fA * mU fU * mG * fG * mG * fA * mG * fG | XXX | |
| | | * mC * T * mU | | |
| WV- | TAGCUUCUUGUCCAGCUU | PO5MRdT * fA * mG fC * mU fU * mC fU * mU fG | XXOXOXOXOXOXOXXXXXX | 896 |
| 7646 | UAUTU | * mU fC * mC fA * mG * fC * mU * fU * mU * fA | XXX | |
| | | * mU * T * mU | | |
| WV- | TGUCCAGCUUUAUUGGGA | PS5MRdT * fG * mU fC * mC fA * mG fC * mU fU | XXOXOXOXOXOXOXXXXXX | 897 |
| 7647 | GGCTU | * mU fA * mU fU * mG * fG * mG * fA * mG * fG | XXX | |
| | | * mC * T * mU | | |
| WV- | TAGCUUCUUGUCCAGCUU | PS5MRdT * fA * mG fC * mU fU * mC fU * mU fG | XXOXOXOXOXOXOXXXXXX | 898 |
| 7648 | UAUTU | * mU fC * mC fA * mG * fC * mU * fU * mU * fA | XXX | |
| | | * mU * T * mU | | |
| WV- | TGUCCAGCUUUAUUGGGA | PH5MRdT * fG * mU fC * mC fA * mG fC * mU fU | XXOXOXOXOXOXOXXXXXX | 899 |
| 7649 | GGCTU | * mU fA * mU fU * mG * fG * mG * fA * mG * fG | XXX | |
| | | * mC * T * mU | | |
| WV- | TAGCUUCUUGUCCAGCUU | PH5MRdT * fA * mG fC * mU fU * mC fU * mU fG | XXOXOXOXOXOXOXXXXXX | 900 |
| 7650 | UAUTU | * mU fC * mC fA * mG * fC * mU * fU * mU * fA | XXX | |
| | | * mU * T * mU | | |
| WV- | TUUGUCCAGCUUUAUUGG | T * fU * mU fG * mU fC * mC fA * mG fC * mU fU | XXOXOXOXOXOXOXXXXXX | 901 |
| 7660 | GAGTU | * mU fA * mU * fU * mG * fG * mG * fA * mG * T | XXX | |
| | | * mU | | |
| WV- | TCUUGUCCAGCUUUAUUG | T * fC * mU fU * mG fU * mC fC * mA fG * mC fU | XXOXOXOXOXOXOXXXXXX | 902 |
| 7661 | GGATU | * mU fU * mA * fU * mU * fG * mG * fG * mA * T | XXX | |
| | | * mU | | |
| WV- | TUCUUGUCCAGCUUUAUU | T * fU * mC fU * mU fG * mU fC * mC fA * mG fC | XXOXOXOXOXOXOXXXXXX | 903 |
| 7662 | GGGTU | * mU fU * mU * fA * mU * fU * mG * fG * mG * T | XXX | |
| | | * mU | | |
| WV- | TUAGCAGCUUCUUGUCCA | T * fU * mA fG * mC fA * mG fC * mU fU * mC fU | XXOXOXOXOXOXOXXXXXX | 904 |
| 7663 | GCUTU | * mU fG * mU * fC * mC * fA * mG * fC * mU * T | XXX | |
| | | * mU | | |
| WV- | TAUAGCAGCUUCUUGUCC | T * fA * mU fA * mG fC * mA fG * mC fU * mU fC | XXOXOXOXOXOXOXXXXXX | 905 |
| 7664 | AGCTU | * mU fU * mG * fU * mC * fC * mA * fG * mC * T | XXX | |
| | | * mU | | |
| WV- | TCAUAGCAGCUUCUUGUC | T * fC * mA fU * mA fG * mC fA * mG fC * mU fU | XXOXOXOXOXOXOXXXXXX | 906 |
| 7665 | CAGTU | * mC fU * mU * fG * mU * fC * mC * fA * mG * T | XXX | |
| | | * mU | | |
| WV- | TUUGUCCAGCUUUAUUGG | T * S fU * SmU fG * SmU fC * SmC fA * SmG fC | * SSOSOSOSOSOSOSSSSSSSS | 907 |
| 7666 | GAGTU | SmU fU * SmU fA * SmU * S fU * SmG * S fG * | S | |
| | | SmG * S fA * SmG * ST * SmU | | |
| WV- | TCUUGUCCAGCUUUAUUG | T * S fC * SmU fU * SmG fU * SmC fC * SmA fG * | SSOSOSOSOSOSOSSSSSSSS | 908 |
| 7667 | GGATU | SmC fU * SmU fU * SmA * S fU * SmU * S fG * | S | |
| | | SmG * S fG * SmA * ST * SmU | | |
| WV- | TUCUUGUCCAGCUUUAUU | T * S fU * SmC fU * SmU fG * SmU fC * SmC fA * | SSOSOSOSOSOSOSSSSSSSS | 909 |
| 7668 | GGGTU | SmG fC * SmU fU * SmU * S fA * SmU * S fU * | S | |
| | | SmG * S fG * SmG * ST * SmU | | |
| WV- | TUAGCAGCUUCUUGUCCA | T * S fU * SmA fG * SmC fA * SmG fC * SmU fU * | SSOSOSOSOSOSOSSSSSSSS | 910 |
| 7669 | GCUTU | SmC fU * SmU fG * SmU * S fC * SmC * S fA * | S | |
| | | SmG * S fC * SmU * ST * SmU | | |
| WV- | TAUAGCAGCUUCUUGUCC | T * S fA * SmU fA * SmG fC * SmA fG * SmC fU * | SSOSOSOSOSOSOSSSSSSSS | 911 |
| 7670 | AGCTU | SmU fC * SmU fU * SmG * S fU * SmC * S fC * | S | |
| | | SmA * S fG * SmC * ST * SmU | | |
| WV- | TCAUAGCAGCUUCUUGUC | T * S fC * SmA fU * SmA fG * SmC fA * SmG fC * | SSOSOSOSOSOSOSSSSSSSS | 912 |
| 7671 | CAGTU | SmU fU * SmC fU * SmU * S fG * SmU * S fC * | S | |
| | | SmC * S fA * SmG * ST * SmU | | |
| WV- | TGUCCAGCUUUAUUGGGA | T * fG * mU * fC * mC * fA * mG * fC * mU * fU * | XXXXXXXXXXXXXXXXXXXXX | 913 |
| 7672 | GGCTU | mU * fA * mU * fU * mG * fG * mG * fA * mG * | X | |
| | | fG * mC * T * mU | | |
| WV- | TAGCUUCUUGUCCAGCUU | T * fA * mG * fC * mU * fU * mC * fU * mU * fG * | XXXXXXXXXXXXXXXXXXXXX | 914 |
| 7673 | UAUTU | mU * fC * mC * fA * mG * fC * mU * fU * mU * | X | |
| | | fA * mU * T * mU | | |
| WV- | CCAGCTTTATTAGGGACAGC | L001m5Ceo * m5Ceo * Aeo * Geo * m5Ceo * T | OXXXXXXXXXXXXXXXXXXX | 915 |
| 493 | | * T * T * A * T * T * A * G * G * G * Aeo * | | |
| | | m5Ceo * Aeo * Geo * m5Ceo | | |
| WV- | CCAGCTTTATTAGGGACAGC | Mod001L001m5Ceo * m5Ceo * Aeo * Geo * | OXXXXXXXXXXXXXXXXXXX | 916 |
| 495 | | m5Ceo * T * T * T * A * T * T * A * G * G * G * | | |
| | | Aeo * m5Ceo * Aeo * Geo * m5Ceo | | |
| WV- | GGAGCAGCTGCCTCTAGGGA | G * G * A * G * C * A * G * C * T * G * C * C * T * | XXXXXXXXXXXXXXXXXXX | 917 |
| 692 | | C * T * A * G * G * G * A | | |
| WV- | TGGAGCAGCTGCCTCTAGGG | T * G * G * A * G * C * A * G * C * T * G * C * C * | XXXXXXXXXXXXXXXXXXX | 918 |
| 693 | | T * C * T * A * G * G * G | | |
| WV- | TGTTCCTGGAGCAGCTGCCT | T * G * T * T * C * C * T * G * G * A * G * C * A * | XXXXXXXXXXXXXXXXXXX | 919 |
| 694 | | G * C * T * G * C * C * T | | |
| WV- | TCCTTGGCGGTCTTGGTGGC | T * C * C * T * T * G * G * C * G * G * T * C * T * | XXXXXXXXXXXXXXXXXXX | 920 |
| 695 | | T * G * G * T * G * G * C | | |
| WV- | ATCCTTGGCGGTCTTGGTGG | A * T * C * C * T * T * G * G * C * G * G * T * C * | XXXXXXXXXXXXXXXXXXX | 921 |
| 696 | | T * T * G * G * T * G * G | | |
| WV- | CATCCTTGGCGGTCTTGGTG | C * A * T * C * C * T * T * G * G * C * G * G * T * | XXXXXXXXXXXXXXXXXXX | 922 |
| 697 | | C * T * T * G * G * T * G | | |
| WV- | GCATCCTTGGCGGTCTTGGT | G * C * A * T * C * C * T * T * G * G * C * G * G * | XXXXXXXXXXXXXXXXXXX | 923 |
| 698 | | T * C * T * T * G * G * T | | |
| WV- | CTGGCCTGCTGGGCCACCTG | C * T * G * G * C * C * T * G * C * T * G * G * G * | XXXXXXXXXXXXXXXXXXX | 924 |
| 699 | | C * C * A * C * C * T * G | | |
| WV- | GGCGGTCTTGGTGGCGTGCT | G * G * C * G * G * T * C * T * T * G * G * T * G * | XXXXXXXXXXXXXXXXXXX | 925 |
| 700 | | G * C * G * T * G * C * T | | |
| WV- | TGGCGGTCTTGGTGGCGTGC | T * G * G * C * G * G * T * C * T * T * G * G * T * | XXXXXXXXXXXXXXXXXXX | 926 |
| 701 | | G * G * C * G * T * G * C | | |
| WV- | TTGGCGGTCTTGGTGGCGTG | T * T * G * G * C * G * G * T * C * T * T * G * G * | XXXXXXXXXXXXXXXXXXX | 927 |
| 702 | | T * G * G * C * G * T * G | | |
| WV- | CTTGGCGGTCTTGGTGGCGT | C * T * T * G * G * C * G * G * T * C * T * T * G * | XXXXXXXXXXXXXXXXXXX | 928 |
| 703 | | G * T * G * G * C * G * T | | |
| WV- | CCTTGGCGGTCTTGGTGGCG | C * C * T * T * G * G * C * G * G * T * C * T * T * | XXXXXXXXXXXXXXXXXXX | 929 |
| 704 | | G * G * T * G * G * C * G | | |
| WV- | GCCCCTGGCCTGCTGGGCCA | G * C * C * C * C * T * G * G * C * C * T * G * C * | XXXXXXXXXXXXXXXXXXX | 930 |
| 705 | | T * G * G * G * C * C * A | | |
| WV- | GGCAGAGGCCAGGAGCGCCA | G * G * C * A * G * A * G * G * C * C * A * G * G | XXXXXXXXXXXXXXXXXXX | 931 |
| 706 | | * A * G * C * G * C * C * A | | |
| WV- | GAGGCATCCTCGGCCTCTGA | G * A * G * G * C * A * T * C * C * T * C * G * G * | XXXXXXXXXXXXXXXXXXX | 932 |
| 707 | | C * C * T * C * T * G * A | | |
| WV- | GGAGGCATCCTCGGCCTCTG | G * G * A * G * G * C * A * T * C * C * T * C * G * | XXXXXXXXXXXXXXXXXXX | 933 |
| 708 | | G * C * C * T * C * T * G | | |
| WV- | GGGAGGCATCCTCGGCCTCT | G * G * G * A * G * G * C * A * T * C * C * T * C * | XXXXXXXXXXXXXXXXXXX | 934 |
| 709 | | G * G * C * C * T * C * T | | |
| WV- | AGGGAGGCATCCTCGGCCTC | A * G * G * G * A * G * G * C * A * T * C * C * T * | XXXXXXXXXXXXXXXXXXX | 935 |
| 710 | | C * G * G * C * C * T * C | | |
| WV- | AAGGGAGGCATCCTCGGCCT | A * A * G * G * G * A * G * G * C * A * T * C * C | XXXXXXXXXXXXXXXXXXX | 936 |
| 711 | | * T * C * G * G * C * C * T | | |
| WV- | GAAGGGAGGCATCCTCGGCC | G * A * A * G * G * G * A * G * G * C * A * T * C | XXXXXXXXXXXXXXXXXXX | 937 |
| 712 | | * C * T * C * G * G * C * C | | |
| WV- | GGTCTTGGTGGCGTGCTTCA | G * G * T * C * T * T * G * G * T * G * G * C * G * | XXXXXXXXXXXXXXXXXXX | 938 |
| 713 | | T * G * C * T * T * C * A | | |
| WV- | GCGGTCTTGGTGGCGTGCTT | G * C * G * G * T * C * T * T * G * G * T * G * G * | XXXXXXXXXXXXXXXXXXX | 939 |
| 714 | | C * G * T * G * C * T * T | | |
| WV- | GTCTCAGGCAGCCACGGCTG | G * T * C * T * C * A * G * G * C * A * G * C * C * | XXXXXXXXXXXXXXXXXXX | 940 |
| 715 | | A * C * G * G * C * T * G | | |
| WV- | AGGCCAGCATGCCTGGAGGG | A * G * G * C * C * A * G * C * A * T * G * C * C * | XXXXXXXXXXXXXXXXXXX | 941 |
| 716 | | T * G * G * A * G * G * G | | |
| WV- | TGCATCCTTGGCGGTCTTGG | T * G * C * A * T * C * C * T * T * G * G * C * G * | XXXXXXXXXXXXXXXXXXX | 942 |
| 717 | | G * T * C * T * T * G * G | | |
| WV- | GTGCATCCTTGGCGGTCTTG | G * T * G * C * A * T * C * C * T * T * G * G * C * | XXXXXXXXXXXXXXXXXXX | 943 |
| 718 | | G * G * T * C * T * T * G | | |
| WV- | CTGCTGGGCCACCTGGGACT | C * T * G * C * T * G * G * G * C * C * A * C * C * | XXXXXXXXXXXXXXXXXXX | 944 |
| 719 | | T * G * G * G * A * C * T | | |
| WV- | TGAAGCCATCGGTCACCCAG | T * G * A * A * G * C * C * A * T * C * G * G * T * | XXXXXXXXXXXXXXXXXXX | 945 |
| 720 | | C * A * C * C * C * A * G | | |
| WV- | CTGAAGCCATCGGTCACCCA | C * T * G * A * A * G * C * C * A * T * C * G * G * | XXXXXXXXXXXXXXXXXXX | 946 |
| 721 | | T * C * A * C * C * C * A | | |
| WV- | CTTGTCCTTAACGGTGCTCC | C * T * T * G * T * C * C * T * T * A * A * C * G * | XXXXXXXXXXXXXXXXXXX | 947 |
| 722 | | G * T * G * C * T * C * C | | |
| WV- | TGTCCAGCTTTATTGGGAGG | T * G * T * C * C * A * G * C * T * T * T * A * T * | XXXXXXXXXXXXXXXXXXX | 948 |
| 723 | | T * G * G * G * A * G * G | | |
| WV- | TGCCTCTAGGGATGAACTGA | T * G * C * C * T * C * T * A * G * G * G * A * T * | XXXXXXXXXXXXXXXXXXX | 949 |
| 724 | | G * A * A * C * T * G * A | | |
| WV- | CTGCATGGCACCTCTGTTCC | C * T * G * C * A * T * G * G * C * A * C * C * T * | XXXXXXXXXXXXXXXXXXX | 950 |
| 725 | | C * T * G * T * T * C * C | | |
| WV- | GGGCTGCATGGCACCTCTGT | G * G * G * C * T * G * C * A * T * G * G * C * A | XXXXXXXXXXXXXXXXXXX | 951 |
| 726 | | * C * C * T * C * T * G * T | | |
| WV- | CTCTGAAGCTCGGGCAGAGG | C * T * C * T * G * A * A * G * C * T * C * G * G * | XXXXXXXXXXXXXXXXXXX | 952 |
| 727 | | G * C * A * G * A * G * G | | |
| WV- | CCTCTGAAGCTCGGGCAGAG | C * C * T * C * T * G * A * A * G * C * T * C * G * | XXXXXXXXXXXXXXXXXXX | 953 |
| 728 | | G * G * C * A * G * A * G | | |
| WV- | GCCTCTGAAGCTCGGGCAGA | G * C * C * T * C * T * G * A * A * G * C * T * C * | XXXXXXXXXXXXXXXXXXX | 954 |
| 729 | | G * G * G * C * A * G * A | | |
| WV- | CGGCCTCTGAAGCTCGGGCA | C * G * G * C * C * T * C * T * G * A * A * G * C * | XXXXXXXXXXXXXXXXXXX | 955 |
| 730 | | T * C * G * G * G * C * A | | |
| WV- | TCGGCCTCTGAAGCTCGGGC | T * C * G * G * C * C * T * C * T * G * A * A * G * | XXXXXXXXXXXXXXXXXXX | 956 |
| 731 | | C * T * C * G * G * G * C | | |
| WV- | CTCGGCCTCTGAAGCTCGGG | C * T * C * G * G * C * C * T * C * T * G * A * A * | XXXXXXXXXXXXXXXXXXX | 957 |
| 732 | | G * C * T * C * G * G * G | | |
| WV- | CCTCGGCCTCTGAAGCTCGG | C * C * T * C * G * G * C * C * T * C * T * G * A * | XXXXXXXXXXXXXXXXXXX | 958 |
| 733 | | A * G * C * T * C * G * G | | |
| WV- | TCCTCGGCCTCTGAAGCTCG | T * C * C * T * C * G * G * C * C * T * C * T * G * | XXXXXXXXXXXXXXXXXXX | 959 |
| 734 | | A * A * G * C * T * C * G | | |
| WV- | TGCTCAGTGCATCCTTGGCG | T * G * C * T * C * A * G * T * G * C * A * T * C * | XXXXXXXXXXXXXXXXXXX | 960 |
| 735 | | C * T * T * G * G * C * G | | |
| WV- | CCTGGGACTCCTGCACGCTG | C * C * T * G * G * G * A * C * T * C * C * T * G * | XXXXXXXXXXXXXXXXXXX | 961 |
| 736 | | C * A * C * G * C * T * G | | |
| WV- | CCACCTGGGACTCCTGCACG | C * C * A * C * C * T * G * G * G * A * C * T * C * | XXXXXXXXXXXXXXXXXXX | 962 |
| 737 | | C * T * G * C * A * C * G | | |
| WV- | TCGGTCACCCAGCCCCTGGC | T * C * G * G * T * C * A * C * C * C * A * G * C * | XXXXXXXXXXXXXXXXXXX | 963 |
| 738 | | C * C * C * T * G * G * C | | |
| WV- | ATCGGTCACCCAGCCCCTGG | A * T * C * G * G * T * C * A * C * C * C * A * G * | XXXXXXXXXXXXXXXXXXX | 964 |
| 739 | | C * C * C * C * T * G * G | | |
| WV- | CATCGGTCACCCAGCCCCTG | C * A * T * C * G * G * T * C * A * C * C * C * A * | XXXXXXXXXXXXXXXXXXX | 965 |
| 740 | | G * C * C * C * C * T * G | | |
| WV- | CCATCGGTCACCCAGCCCCT | C * C * A * T * C * G * G * T * C * A * C * C * C * | XXXXXXXXXXXXXXXXXXX | 966 |
| 741 | | A * G * C * C * C * C * T | | |
| WV- | GCCATCGGTCACCCAGCCCC | G * C * C * A * T * C * G * G * T * C * A * C * C * | XXXXXXXXXXXXXXXXXXX | 967 |
| 742 | | C * A * G * C * C * C * C | | |
| WV- | AGCCATCGGTCACCCAGCCC | A * G * C * C * A * T * C * G * G * T * C * A * C * | XXXXXXXXXXXXXXXXXXX | 968 |
| 743 | | C * C * A * G * C * C * C | | |
| WV- | TCCAGCTTTATTGGGAGGCC | T * C * C * A * G * C * T * T * T * A * T * T * G * | XXXXXXXXXXXXXXXXXXX | 969 |
| 744 | | G * G * A * G * G * C * C | | |
| WV- | CATCCTCGGCCTCTGAAGCT | C * A * T * C * C * T * C * G * G * C * C * T * C * | XXXXXXXXXXXXXXXXXXX | 970 |
| 745 | | T * G * A * A * G * C * T | | |
| WV- | AGGCATCCTCGGCCTCTGAA | A * G * G * C * A * T * C * C * T * C * G * G * C * | XXXXXXXXXXXXXXXXXXX | 971 |
| 746 | | C * T * C * T * G * A * A | | |
| WV- | TCTTGGTGGCGTGCTTCATG | T * C * T * T * G * G * T * G * G * C * G * T * G * | XXXXXXXXXXXXXXXXXXX | 972 |
| 747 | | C * T * T * C * A * T * G | | |
| WV- | CACGCTGCTCAGTGCATCCT | C * A * C * G * C * T * G * C * T * C * A * G * T * | XXXXXXXXXXXXXXXXXXX | 973 |
| 748 | | G * C * A * T * C * C * T | | |
| WV- | CTCCTGCACGCTGCTCAGTG | C * T * C * C * T * G * C * A * C * G * C * T * G * | XXXXXXXXXXXXXXXXXXX | 974 |
| 749 | | C * T * C * A * G * T * G | | |
| WV- | GGACTCCTGCACGCTGCTCA | G * G * A * C * T * C * C * T * G * C * A * C * G * | XXXXXXXXXXXXXXXXXXX | 975 |
| 750 | | C * T * G * C * T * C * A | | |
| WV- | GGGACTCCTGCACGCTGCTC | G * G * G * A * C * T * C * C * T * G * C * A * C * | XXXXXXXXXXXXXXXXXXX | 976 |
| 751 | | G * C * T * G * C * T * C | | |
| WV- | TGGGACTCCTGCACGCTGCT | T * G * G * G * A * C * T * C * C * T * G * C * A * | XXXXXXXXXXXXXXXXXXX | 977 |
| 752 | | C * G * C * T * G * C * T | | |
| WV- | AGGTCTCAGGCAGCCACGGC | A * G * G * T * C * T * C * A * G * G * C * A * G * | XXXXXXXXXXXXXXXXXXX | 978 |
| 753 | | C * C * A * C * G * G * C | | |
| WV- | GAGGTCTCAGGCAGCCACGG | G * A * G * G * T * C * T * C * A * G * G * C * A * | XXXXXXXXXXXXXXXXXXX | 979 |
| 754 | | G * C * C * A * C * G * G | | |
| WV- | TGAGGTCTCAGGCAGCCACG | T * G * A * G * G * T * C * T * C * A * G * G * C * | XXXXXXXXXXXXXXXXXXX | 980 |
| 755 | | A * G * C * C * A * C * G | | |
| WV- | CCTGGAGATTGCAGGACCCA | C * C * T * G * G * A * G * A * T * T * G * C * A * | XXXXXXXXXXXXXXXXXXX | 981 |
| 756 | | G * G * A * C * C * C * A | | |
| WV- | GCCCTGGAGATTGCAGGACC | G * C * C * C * T * G * G * A * G * A * T * T * G * | XXXXXXXXXXXXXXXXXXX | 982 |
| 757 | | C * A * G * G * A * C * C | | |
| WV- | CCAGGAGCGCCAGGAGGGCA | C * C * A * G * G * A * G * C * G * C * C * A * G | XXXXXXXXXXXXXXXXXXX | 983 |
| 758 | | * G * A * G * G * G * C * A | | |
| WV- | CGTGCTTCATGTAACCCTGC | C * G * T * G * C * T * T * C * A * T * G * T * A * | XXXXXXXXXXXXXXXXXXX | 984 |
| 759 | | A * C * C * C * T * G * C | | |
| WV- | TGGTCTGACCTCAGGGTCCA | T * G * G * T * C * T * G * A * C * C * T * C * A * | XXXXXXXXXXXXXXXXXXX | 985 |
| 760 | | G * G * G * T * C * C * A | | |
| WV- | TTGGTCTGACCTCAGGGTCC | T * T * G * G * T * C * T * G * A * C * C * T * C * | XXXXXXXXXXXXXXXXXXX | 986 |
| 761 | | A * G * G * G * T * C * C | | |
| WV- | AAGTTGGTCTGACCTCAGGG | A * A * G * T * T * G * G * T * C * T * G * A * C * | XXXXXXXXXXXXXXXXXXX | 987 |
| 762 | | C * T * C * A * G * G * G | | |
| WV- | TGAAGTTGGTCTGACCTCAG | T * G * A * A * G * T * T * G * G * T * C * T * G * | XXXXXXXXXXXXXXXXXXX | 988 |
| 763 | | A * C * C * T * C * A * G | | |
| WV- | CACGGCTGAAGTTGGTCTGA | C * A * C * G * G * C * T * G * A * A * G * T * T * | XXXXXXXXXXXXXXXXXXX | 989 |
| 764 | | G * G * T * C * T * G * A | | |
| WV- | CCACGGCTGAAGTTGGTCTG | C * C * A * C * G * G * C * T * G * A * A * G * T * | XXXXXXXXXXXXXXXXXXX | 990 |
| 765 | | T * G * G * T * C * T * G | | |
| WV- | GCCACGGCTGAAGTTGGTCT | G * C * C * A * C * G * G * C * T * G * A * A * G * | XXXXXXXXXXXXXXXXXXX | 991 |
| 766 | | T * T * G * G * T * C * T | | |
| WV- | AGCCACGGCTGAAGTTGGTC | A * G * C * C * A * C * G * G * C * T * G * A * A * | XXXXXXXXXXXXXXXXXXX | 992 |
| 767 | | G * T * T * G * G * T * C | | |
| WV- | GTCTTGGTGGCGTGCTTCAT | G * T * C * T * T * G * G * T * G * G * C * G * T * | XXXXXXXXXXXXXXXXXXX | 993 |
| 768 | | G * C * T * T * C * A * T | | |
| WV- | CAGTGCATCCTTGGCGGTCT | C * A * G * T * G * C * A * T * C * C * T * T * G * | XXXXXXXXXXXXXXXXXXX | 994 |
| 769 | | G * C * G * G * T * C * T | | |
| WV- | CTGCCTCTAGGGATGAACTG | C * T * G * C * C * T * C * T * A * G * G * G * A * | XXXXXXXXXXXXXXXXXXX | 995 |
| 770 | | T * G * A * A * C * T * G | | |
| WV- | GGCATCCTCGGCCTCTGAAG | G * G * C * A * T * C * C * T * C * G * G * C * C * | XXXXXXXXXXXXXXXXXXX | 996 |
| 771 | | T * C * T * G * A * A * G | | |
| WV- | TTGGTGGCGTGCTTCATGTA | T * T * G * G * T * G * G * C * G * T * G * C * T * | XXXXXXXXXXXXXXXXXXX | 997 |
| 772 | | T * C * A * T * G * T * A | | |
| WV- | GCGTGCTTCATGTAACCCTG | G * C * G * T * G * C * T * T * C * A * T * G * T * | XXXXXXXXXXXXXXXXXXX | 998 |
| 773 | | A * A * C * C * C * T * G | | |
| WV- | TGAGAAGGGAGGCATCCTCG | T * G * A * G * A * A * G * G * G * A * G * G * C | XXXXXXXXXXXXXXXXXXX | 999 |
| 774 | | * A * T * C * C * T * C * G | | |
| WV- | GCTGAAGTTGGTCTGACCTC | G * C * T * G * A * A * G * T * T * G * G * T * C * | XXXXXXXXXXXXXXXXXXX | 1000 |
| 775 | | T * G * A * C * C * T * C | | |
| WV- | GGGCCTCCCAAGGCAAACCC | G * G * G * C * C * T * C * C * C * A * A * G * G * | XXXXXXXXXXXXXXXXXXX | 1001 |
| 776 | | C * A * A * A * C * C * C | | |
| WV- | GTTTATGCCCCTGGGCCTGA | G * T * T * T * A * T * G * C * C * C * C * T * G * | XXXXXXXXXXXXXXXXXXX | 1002 |
| 777 | | G * G * C * C * T * G * A | | |
| WV- | AACCTTAGCTGGGTCTGCCA | A * A * C * C * T * T * A * G * C * T * G * G * G * | XXXXXXXXXXXXXXXXXXX | 1003 |
| 778 | | T * C * T * G * C * C * A | | |
| WV- | CACCCATTGGGACTGGGATC | C * A * C * C * C * A * T * T * G * G * G * A * C * | XXXXXXXXXXXXXXXXXXX | 1004 |
| 779 | | T * G * G * G * A * T * C | | |
| WV- | CTCCTGCTTGACCACCCATT | C * T * C * C * T * G * C * T * T * G * A * C * C * | XXXXXXXXXXXXXXXXXXX | 1005 |
| 780 | | A * C * C * C * A * T * T | | |
| WV- | GCTCCTGCTTGACCACCCAT | G * C * T * C * C * T * G * C * T * T * G * A * C * | XXXXXXXXXXXXXXXXXXX | 1006 |
| 781 | | C * A * C * C * C * A * T | | |
| WV- | TGGGCTCCTGCTTGACCACC | T * G * G * G * C * T * C * C * T * G * C * T * T * | XXXXXXXXXXXXXXXXXXX | 1007 |
| 782 | | G * A * C * C * A * C * C | | |
| WV- | GCCTGACAAAGGCCCTGTGA | G * C * C * T * G * A * C * A * A * A * G * G * C * | XXXXXXXXXXXXXXXXXXX | 1008 |
| 783 | | C * C * T * G * T * G * A | | |
| WV- | TCCTTGCAGGAACCCCAGCA | T * C * C * T * T * G * C * A * G * G * A * A * C * | XXXXXXXXXXXXXXXXXXX | 1009 |
| 784 | | C * C * C * A * G * C * A | | |
| WV- | ACACCACCCTCTCAACTTCA | A * C * A * C * C * A * C * C * C * T * C * T * C * | XXXXXXXXXXXXXXXXXXX | 1010 |
| 785 | | A * A * C * T * T * C * A | | |
| WV- | ACACCCATGTCCCCACTGGA | A * C * A * C * C * C * A * T * G * T * C * C * C * | XXXXXXXXXXXXXXXXXXX | 1011 |
| 786 | | C * A * C * T * G * G * A | | |
| WV- | TGAGAACTCCTCTGTAGGCA | T * G * A * G * A * A * C * T * C * C * T * C * T * | XXXXXXXXXXXXXXXXXXX | 1012 |
| 787 | | G * T * A * G * G * C * A | | |
| WV- | GGAGCAGCTGCCTCTAGGGA | mG * mG * mA * mG * mC * A * G * C * T * G * | XXXXXXXXXXXXXXXXXXX | 1013 |
| 788 | | C * C * T * C * T * A * G * G * G * A | | |
| WV- | UGGAGCAGCTGCCTCTAGGG | mU * mG * mG * mA * mG * C * A * G * C * T * | XXXXXXXXXXXXXXXXXXX | 1014 |
| 789 | | G * C * C * T * C * T * A * G * G * G | | |
| WV- | UGUUCCTGGAGCAGCTGCCT | mU * mG * mU * mU * mC * C * T * G * G * A * | XXXXXXXXXXXXXXXXXXX | 1015 |
| 790 | | G * C * A * G * C * T * G * C * C * T | | |
| WV- | UCCUUGGCGGTCTTGGTGGC | mU * mC * mC * mU * mU * G * G * C * G * G * | XXXXXXXXXXXXXXXXXXX | 1016 |
| 791 | | T * C * T * T * G * G * T * G * G * C | | |
| WV- | AUCCUTGGCGGTCTTGGTGG | mA * mU * mC * mC * mU * T * G * G * C * G * | XXXXXXXXXXXXXXXXXXX | 1017 |
| 792 | | G * T * C * T * T * G * G * T * G * G | | |
| WV- | CAUCCTTGGCGGTCTTGGTG | mC * mA * mU * mC * mC * T * T * G * G * C * | XXXXXXXXXXXXXXXXXXX | 1018 |
| 793 | | G * G * T * C * T * T * G * G * T * G | | |
| WV- | GCAUCCTTGGCGGTCTTGGT | mG * mC * mA * mU * mC * C * T * T * G * G * | XXXXXXXXXXXXXXXXXXX | 1019 |
| 794 | | C * G * G * T * C * T * T * G * G * T | | |
| WV- | CUGGCCTGCTGGGCCACCTG | mC * mU * mG * mG * mC * C * T * G * C * T * | XXXXXXXXXXXXXXXXXXX | 1020 |
| 795 | | G * G * G * C * C * A * C * C * T * G | | |
| WV- | GGCGGTCTTGGTGGCGTGCT | mG * mG * mC * mG * mG * T * C * T * T * G * | XXXXXXXXXXXXXXXXXXX | 1021 |
| 796 | | G * T * G * G * C * G * T * G * C * T | | |
| WV- | UGGCGGTCTTGGTGGCGTGC | mU * mG * mG * mC * mG * G * T * C * T * T * | XXXXXXXXXXXXXXXXXXX | 1022 |
| 797 | | G * G * T * G * G * C * G * T * G * C | | |
| WV- | UUGGCGGTCTTGGTGGCGTG | mU * mU * mG * mG * mC * G * G * T * C * T * | XXXXXXXXXXXXXXXXXXX | 1023 |
| 798 | | T * G * G * T * G * G * C * G * T * G | | |
| WV- | CUUGGCGGTCTTGGTGGCGT | mC * mU * mU * mG * mG * C * G * G * T * C * | XXXXXXXXXXXXXXXXXXX | 1024 |
| 799 | | T * T * G * G * T * G * G * C * G * T | | |
| WV- | CCUUGGCGGTCTTGGTGGCG | mC * mC * mU * mU * mG * G * C * G * G * T * | XXXXXXXXXXXXXXXXXXX | 1025 |
| 800 | | C * T * T * G * G * T * G * G * C * G | | |
| WV- | GCCCCTGGCCTGCTGGGCCA | mG * mC * mC * mC * mC * T * G * G * C * C * T | XXXXXXXXXXXXXXXXXXX | 1026 |
| 801 | | * G * C * T * G * G * G * C * C * A | | |
| WV- | GGCAGAGGCCAGGAGCGCCA | mG * mG * mC * mA * mG * A * G * G * C * C * | XXXXXXXXXXXXXXXXXXX | 1027 |
| 802 | | A * G * G * A * G * C * G * C * C * A | | |
| WV- | GAGGCATCCTCGGCCTCTGA | mG * mA * mG * mG * mC * A * T * C * C * T * | XXXXXXXXXXXXXXXXXXX | 1028 |
| 803 | | C * G * G * C * C * T * C * T * G * A | | |
| WV- | GGAGGCATCCTCGGCCTCTG | mG * mG * mA * mG * mG * C * A * T * C * C * | XXXXXXXXXXXXXXXXXXX | 1029 |
| 804 | | T * C * G * G * C * C * T * C * T * G | | |
| WV- | GGGAGGCATCCTCGGCCTCT | mG * mG * mG * mA * mG * G * C * A * T * C * | XXXXXXXXXXXXXXXXXXX | 1030 |
| 805 | | C * T * C * G * G * C * C * T * C * T | | |
| WV- | AGGGAGGCATCCTCGGCCTC | mA * mG * mG * mG * mA * G * G * C * A * T * | XXXXXXXXXXXXXXXXXXX | 1031 |
| 806 | | C * C * T * C * G * G * C * C * T * C | | |
| WV- | AAGGGAGGCATCCTCGGCCT | mA * mA * mG * mG * mG * A * G * G * C * A * | XXXXXXXXXXXXXXXXXXX | 1032 |
| 807 | | T * C * C * T * C * G * G * C * C * T | | |
| WV- | GAAGGGAGGCATCCTCGGCC | mG * mA * mA * mG * mG * G * A * G * G * C * | XXXXXXXXXXXXXXXXXXX | 1033 |
| 808 | | A * T * C * C * T * C * G * G * C * C | | |
| WV- | GGUCUTGGTGGCGTGCTTCA | mG * mG * mU * mC * mU * T * G * G * T * G * | XXXXXXXXXXXXXXXXXXX | 1034 |
| 809 | | G * C * G * T * G * C * T * T * C * A | | |
| WV- | GCGGUCTTGGTGGCGTGCTT | mG * mC * mG * mG * mU * C * T * T * G * G * | XXXXXXXXXXXXXXXXXXX | 1035 |
| 810 | | T * G * G * C * G * T * G * C * T * T | | |
| WV- | GUCUCAGGCAGCCACGGCTG | mG * mU * mC * mU * mC * A * G * G * C * A * | XXXXXXXXXXXXXXXXXXX | 1036 |
| 811 | | G * C * C * A * C * G * G * C * T * G | | |
| WV- | AGGCCAGCATGCCTGGAGGG | mA * mG * mG * mC * mC * A * G * C * A * T * | XXXXXXXXXXXXXXXXXXX | 1037 |
| 812 | | G * C * C * T * G * G * A * G * G * G | | |
| WV- | UGCAUCCTTGGCGGTCTTGG | mU * mG * mC * mA * mU * C * C * T * T * G * | XXXXXXXXXXXXXXXXXXX | 1038 |
| 813 | | G * C * G * G * T * C * T * T * G * G | | |
| WV- | GUGCATCCTTGGCGGTCTTG | mG * mU * mG * mC * mA * T * C * C * T * T * | XXXXXXXXXXXXXXXXXXX | 1039 |
| 814 | | G * G * C * G * G * T * C * T * T * G | | |
| WV- | CUGCUGGGCCACCTGGGACT | mC * mU * mG * mC * mU * G * G * G * C * C * | XXXXXXXXXXXXXXXXXXX | 1040 |
| 815 | | A * C * C * T * G * G * G * A * C * T | | |
| WV- | UGAAGCCATCGGTCACCCAG | mU * mG * mA * mA * mG * C * C * A * T * C * | XXXXXXXXXXXXXXXXXXX | 1041 |
| 816 | | G * G * T * C * A * C * C * C * A * G | | |
| WV- | CUGAAGCCATCGGTCACCCA | mC * mU * mG * mA * mA * G * C * C * A * T * | XXXXXXXXXXXXXXXXXXX | 1042 |
| 817 | | C * G * G * T * C * A * C * C * C * A | | |
| WV- | CUUGUCCTTAACGGTGCTCC | mC * mU * mU * mG * mU * C * C * T * T * A * | XXXXXXXXXXXXXXXXXXX | 1043 |
| 818 | | A * C * G * G * T * G * C * T * C * C | | |
| WV- | UGUCCAGCTTTATTGGGAGG | mU * mG * mU * mC * mC * A * G * C * T * T * | XXXXXXXXXXXXXXXXXXX | 1044 |
| 819 | | T * A * T * T * G * G * G * A * G * G | | |
| WV- | UGCCUCTAGGGATGAACTGA | mU * mG * mC * mC * mU * C * T * A * G * G * | XXXXXXXXXXXXXXXXXXX | 1045 |
| 820 | | G * A * T * G * A * A * C * T * G * A | | |
| WV- | CUGCATGGCACCTCTGTTCC | mC * mU * mG * mC * mA * T * G * G * C * A * | XXXXXXXXXXXXXXXXXXX | 1046 |
| 821 | | C * C * T * C * T * G * T * T * C * C | | |
| WV- | GGGCUGCATGGCACCTCTGT | mG * mG * mG * mC * mU * G * C * A * T * G * | XXXXXXXXXXXXXXXXXXX | 1047 |
| 822 | | G * C * A * C * C * T * C * T * G * T | | |
| WV- | CUCUGAAGCTCGGGCAGAGG | mC * mU * mC * mU * mG * A * A * G * C * T * | XXXXXXXXXXXXXXXXXXX | 1048 |
| 823 | | C * G * G * G * C * A * G * A * G * G | | |
| WV- | CCUCUGAAGCTCGGGCAGAG | mC * mC * mU * mC * mU * G * A * A * G * C * | XXXXXXXXXXXXXXXXXXX | 1049 |
| 824 | | T * C * G * G * G * C * A * G * A * G | | |
| WV- | GCCUCTGAAGCTCGGGCAGA | mG * mC * mC * mU * mC * T * G * A * A * G * | XXXXXXXXXXXXXXXXXXX | 1050 |
| 825 | | C * T * C * G * G * G * C * A * G * A | | |
| WV- | CGGCCTCTGAAGCTCGGGCA | mC * mG * mG * mC * mC * T * C * T * G * A * | XXXXXXXXXXXXXXXXXXX | 1051 |
| 826 | | A * G * C * T * C * G * G * G * C * A | | |
| WV- | UCGGCCTCTGAAGCTCGGGC | mU * mC * mG * mG * mC * C * T * C * T * G * | XXXXXXXXXXXXXXXXXXX | 1052 |
| 827 | | A * A * G * C * T * C * G * G * G * C | | |
| WV- | CUCGGCCTCTGAAGCTCGGG | mC * mU * mC * mG * mG * C * C * T * C * T * | XXXXXXXXXXXXXXXXXXX | 1053 |
| 828 | | G * A * A * G * C * T * C * G * G * G | | |
| WV- | CCUCGGCCTCTGAAGCTCGG | mC * mC * mU * mC * mG * G * C * C * T * C * T | XXXXXXXXXXXXXXXXXXX | 1054 |
| 829 | | * G * A * A * G * C * T * C * G * G | | |
| WV- | UCCUCGGCCTCTGAAGCTCG | mU * mC * mC * mU * mC * G * G * C * C * T * | XXXXXXXXXXXXXXXXXXX | 1055 |
| 830 | | C * T * G * A * A * G * C * T * C * G | | |
| WV- | UGCUCAGTGCATCCTTGGCG | mU * mG * mC * mU * mC * A * G * T * G * C * | XXXXXXXXXXXXXXXXXXX | 1056 |
| 831 | | A * T * C * C * T * T * G * G * C * G | | |
| WV- | CCUGGGACTCCTGCACGCTG | mC * mC * mU * mG * mG * G * A * C * T * C * | XXXXXXXXXXXXXXXXXXX | 1057 |
| 832 | | C * T * G * C * A * C * G * C * T * G | | |
| WV- | CCACCTGGGACTCCTGCACG | mC * mC * mA * mC * mC * T * G * G * G * A * | XXXXXXXXXXXXXXXXXXX | 1058 |
| 833 | | C * T * C * C * T * G * C * A * C * G | | |
| WV- | UCGGUCACCCAGCCCCTGGC | mU * mC * mG * mG * mU * C * A * C * C * C * | XXXXXXXXXXXXXXXXXXX | 1059 |
| 834 | | A * G * C * C * C * C * T * G * G * C | | |
| WV- | AUCGGTCACCCAGCCCCTGG | mA * mU * mC * mG * mG * T * C * A * C * C * | XXXXXXXXXXXXXXXXXXX | 1060 |
| 835 | | C * A * G * C * C * C * C * T * G * G | | |
| WV- | CAUCGGTCACCCAGCCCCTG | mC * mA * mU * mC * mG * G * T * C * A * C * | XXXXXXXXXXXXXXXXXXX | 1061 |
| 836 | | C * C * A * G * C * C * C * C * T * G | | |
| WV- | CCAUCGGTCACCCAGCCCCT | mC * mC * mA * mU * mC * G * G * T * C * A * | XXXXXXXXXXXXXXXXXXX | 1062 |
| 837 | | C * C * C * A * G * C * C * C * C * T | | |
| WV- | GCCAUCGGTCACCCAGCCCC | mG * mC * mC * mA * mU * C * G * G * T * C * | XXXXXXXXXXXXXXXXXXX | 1063 |
| 838 | | A * C * C * C * A * G * C * C * C * C | | |
| WV- | AGCCATCGGTCACCCAGCCC | mA * mG * mC * mC * mA * T * C * G * G * T * | XXXXXXXXXXXXXXXXXXX | 1064 |
| 839 | | C * A * C * C * C * A * G * C * C * C | | |
| WV- | UCCAGCTTTATTGGGAGGCC | mU * mC * mC * mA * mG * C * T * T * T * A * T | XXXXXXXXXXXXXXXXXXX | 1065 |
| 840 | | * T * G * G * G * A * G * G * C * C | | |
| WV- | CAUCCTCGGCCTCTGAAGCT | mC * mA * mU * mC * mC * T * C * G * G * C * | XXXXXXXXXXXXXXXXXXX | 1066 |
| 841 | | C * T * C * T * G * A * A * G * C * T | | |
| WV- | AGGCATCCTCGGCCTCTGAA | mA * mG * mG * mC * mA * T * C * C * T * C * | XXXXXXXXXXXXXXXXXXX | 1067 |
| 842 | | G * G * C * C * T * C * T * G * A * A | | |
| WV- | UCUUGGTGGCGTGCTTCATG | mU * mC * mU * mU * mG * G * T * G * G * C * | XXXXXXXXXXXXXXXXXXX | 1068 |
| 843 | | G * T * G * C * T * T * C * A * T * G | | |
| WV- | CACGCTGCTCAGTGCATCCT | mC * mA * mC * mG * mC * T * G * C * T * C * A | XXXXXXXXXXXXXXXXXXX | 1069 |
| 844 | | * G * T * G * C * A * T * C * C * T | | |
| WV- | CUCCUGCACGCTGCTCAGTG | mC * mU * mC * mC * mU * G * C * A * C * G * | XXXXXXXXXXXXXXXXXXX | 1070 |
| 845 | | C * T * G * C * T * C * A * G * T * G | | |
| WV- | GGACUCCTGCACGCTGCTCA | mG * mG * mA * mC * mU * C * C * T * G * C * | XXXXXXXXXXXXXXXXXXX | 1071 |
| 846 | | A * C * G * C * T * G * C * T * C * A | | |
| WV- | GGGACTCCTGCACGCTGCTC | mG * mG * mG * mA * mC * T * C * C * T * G * | XXXXXXXXXXXXXXXXXXX | 1072 |
| 847 | | C * A * C * G * C * T * G * C * T * C | | |
| WV- | UGGGACTCCTGCACGCTGCT | mU * mG * mG * mG * mA * C * T * C * C * T * | XXXXXXXXXXXXXXXXXXX | 1073 |
| 848 | | G * C * A * C * G * C * T * G * C * T | | |
| WV- | AGGUCTCAGGCAGCCACGGC | mA * mG * mG * mU * mC * T * C * A * G * G * | XXXXXXXXXXXXXXXXXXX | 1074 |
| 849 | | C * A * G * C * C * A * C * G * G * C | | |
| WV- | GAGGUCTCAGGCAGCCACGG | mG * mA * mG * mG * mU * C * T * C * A * G * | XXXXXXXXXXXXXXXXXXX | 1075 |
| 850 | | G * C * A * G * C * C * A * C * G * G | | |
| WV- | UGAGGTCTCAGGCAGCCACG | mU * mG * mA * mG * mG * T * C * T * C * A * | XXXXXXXXXXXXXXXXXXX | 1076 |
| 851 | | G * G * C * A * G * C * C * A * C * G | | |
| WV- | CCUGGAGATTGCAGGACCCA | mC * mC * mU * mG * mG * A * G * A * T * T * | XXXXXXXXXXXXXXXXXXX | 1077 |
| 852 | | G * C * A * G * G * A * C * C * C * A | | |
| WV- | GCCCUGGAGATTGCAGGACC | mG * mC * mC * mC * mU * G * G * A * G * A * | XXXXXXXXXXXXXXXXXXX | 1078 |
| 853 | | T * T * G * C * A * G * G * A * C * C | | |
| WV- | CCAGGAGCGCCAGGAGGGCA | mC * mC * mA * mG * mG * A * G * C * G * C * | XXXXXXXXXXXXXXXXXXX | 1079 |
| 854 | | C * A * G * G * A * G * G * G * C * A | | |
| WV- | CGUGCTTCATGTAACCCTGC | mC * mG * mU * mG * mC * T * T * C * A * T * | XXXXXXXXXXXXXXXXXXX | 1080 |
| 855 | | G * T * A * A * C * C * C * T * G * C | | |
| WV- | UGGUCTGACCTCAGGGTCCA | mU * mG * mG * mU * mC * T * G * A * C * C * | XXXXXXXXXXXXXXXXXXX | 1081 |
| 856 | | T * C * A * G * G * G * T * C * C * A | | |
| WV- | UUGGUCTGACCTCAGGGTCC | mU * mU * mG * mG * mU * C * T * G * A * C * | XXXXXXXXXXXXXXXXXXX | 1082 |
| 857 | | C * T * C * A * G * G * G * T * C * C | | |
| WV- | AAGUUGGTCTGACCTCAGGG | mA * mA * mG * mU * mU * G * G * T * C * T * | XXXXXXXXXXXXXXXXXXX | 1083 |
| 858 | | G * A * C * C * T * C * A * G * G * G | | |
| WV- | UGAAGTTGGTCTGACCTCAG | mU * mG * mA * mA * mG * T * T * G * G * T * | XXXXXXXXXXXXXXXXXXX | 1084 |
| 859 | | C * T * G * A * C * C * T * C * A * G | | |
| WV- | CACGGCTGAAGTTGGTCTGA | mC * mA * mC * mG * mG * C * T * G * A * A * | XXXXXXXXXXXXXXXXXXX | 1085 |
| 860 | | G * T * T * G * G * T * C * T * G * A | | |
| WV- | CCACGGCTGAAGTTGGTCTG | mC * mC * mA * mC * mG * G * C * T * G * A * | XXXXXXXXXXXXXXXXXXX | 1086 |
| 861 | | A * G * T * T * G * G * T * C * T * G | | |
| WV- | GCCACGGCTGAAGTTGGTCT | mG * mC * mC * mA * mC * G * G * C * T * G * | XXXXXXXXXXXXXXXXXXX | 1087 |
| 862 | | A * A * G * T * T * G * G * T * C * T | | |
| WV- | AGCCACGGCTGAAGTTGGTC | mA * mG * mC * mC * mA * C * G * G * C * T * | XXXXXXXXXXXXXXXXXXX | 1088 |
| 863 | | G * A * A * G * T * T * G * G * T * C | | |
| WV- | GUCUUGGTGGCGTGCTTCAT | mG * mU * mC * mU * mU * G * G * T * G * G * | XXXXXXXXXXXXXXXXXXX | 1089 |
| 864 | | C * G * T * G * C * T * T * C * A * T | | |
| WV- | CAGUGCATCCTTGGCGGTCT | mC * mA * mG * mU * mG * C * A * T * C * C * | XXXXXXXXXXXXXXXXXXX | 1090 |
| 865 | | T * T * G * G * C * G * G * T * C * T | | |
| WV- | CUGCCTCTAGGGATGAACTG | mC * mU * mG * mC * mC * T * C * T * A * G * | XXXXXXXXXXXXXXXXXXX | 1091 |
| 866 | | G * G * A * T * G * A * A * C * T * G | | |
| WV- | GGCAUCCTCGGCCTCTGAAG | mG * mG * mC * mA * mU * C * C * T * C * G * | XXXXXXXXXXXXXXXXXXX | 1092 |
| 867 | | G * C * C * T * C * T * G * A * A * G | | |
| WV- | UUGGUGGCGTGCTTCATGTA | mU * mU * mG * mG * mU * G * G * C * G * T * | XXXXXXXXXXXXXXXXXXX | 1093 |
| 868 | | G * C * T * T * C * A * T * G * T * A | | |
| WV- | GCGUGCTTCATGTAACCCTG | mG * mC * mG * mU * mG * C * T * T * C * A * | XXXXXXXXXXXXXXXXXXX | 1094 |
| 869 | | T * G * T * A * A * C * C * C * T * G | | |
| WV- | UGAGAAGGGAGGCATCCTCG | mU * mG * mA * mG * mA * A * G * G * G * A * | XXXXXXXXXXXXXXXXXXX | 1095 |
| 870 | | G * G * C * A * T * C * C * T * C * G | | |
| WV- | GCUGAAGTTGGTCTGACCTC | mG * mC * mU * mG * mA * A * G * T * T * G * | XXXXXXXXXXXXXXXXXXX | 1096 |
| 871 | | G * T * C * T * G * A * C * C * T * C | | |
| WV- | GGGCCTCCCAAGGCAAACCC | mG * mG * mG * mC * mC * T * C * C * C * A * | XXXXXXXXXXXXXXXXXXX | 1097 |
| 872 | | A * G * G * C * A * A * A * C * C * C | | |
| WV- | GUUUATGCCCCTGGGCCTGA | mG * mU * mU * mU * mA * T * G * C * C * C * | XXXXXXXXXXXXXXXXXXX | 1098 |
| 873 | | C * T * G * G * G * C * C * T * G * A | | |
| WV- | AACCUTAGCTGGGTCTGCCA | mA * mA * mC * mC * mU * T * A * G * C * T * | XXXXXXXXXXXXXXXXXXX | 1099 |
| 874 | | G * G * G * T * C * T * G * C * C * A | | |
| WV- | CACCCATTGGGACTGGGATC | mC * mA * mC * mC * mC * A * T * T * G * G * | XXXXXXXXXXXXXXXXXXX | 1100 |
| 875 | | G * A * C * T * G * G * G * A * T * C | | |
| WV- | CUCCUGCTTGACCACCCATT | mC * mU * mC * mC * mU * G * C * T * T * G * | XXXXXXXXXXXXXXXXXXX | 1101 |
| 876 | | A * C * C * A * C * C * C * A * T * T | | |
| WV- | GCUCCTGCTTGACCACCCAT | mG * mC * mU * mC * mC * T * G * C * T * T * G | XXXXXXXXXXXXXXXXXXX | 1102 |
| 877 | | * A * C * C * A * C * C * C * A * T | | |
| WV- | UGGGCTCCTGCTTGACCACC | mU * mG * mG * mG * mC * T * C * C * T * G * | XXXXXXXXXXXXXXXXXXX | 1103 |
| 878 | | C * T * T * G * A * C * C * A * C * C | | |
| WV- | GCCUGACAAAGGCCCTGTGA | mG * mC * mC * mU * mG * A * C * A * A * A * | XXXXXXXXXXXXXXXXXXX | 1104 |
| 879 | | G * G * C * C * C * T * G * T * G * A | | |
| WV- | UCCUUGCAGGAACCCCAGCA | mU * mC * mC * mU * mU * G * C * A * G * G * | XXXXXXXXXXXXXXXXXXX | 1105 |
| 880 | | A * A * C * C * C * C * A * G * C * A | | |
| WV- | ACACCACCCTCTCAACTTCA | mA * mC * mA * mC * mC * A * C * C * C * T * C | XXXXXXXXXXXXXXXXXXX | 1106 |
| 881 | | * T * C * A * A * C * T * T * C * A | | |
| WV- | ACACCCATGTCCCCACTGGA | mA * mC * mA * mC * mC * C * A * T * G * T * C | XXXXXXXXXXXXXXXXXXX | 1107 |
| 882 | | * C * C * C * A * C * T * G * G * A | | |
| WV- | UGAGAACTCCTCTGTAGGCA | mU * mG * mA * mG * mA * A * C * T * C * C * | XXXXXXXXXXXXXXXXXXX | 1108 |
| 883 | | T * C * T * G * T * A * G * G * C * A | | |
| WV- | UCCUUGGCGGTCTTGGUGGC | mU * mCmCmUmU * G * G * C * G * G * T * C * | XOOOXXXXXXXXXXXOOOX | 1109 |
| 1850 | | T * T * G * mGmUmGmG * mC | | |
| WV- | AUCCUTGGCGGTCTTGGUGG | mA * mUmCmCmU * T * G * G * C * G * G * T * | XOOOXXXXXXXXXXXOOOX | 1110 |
| 1851 | | C * T * T * mGmGmUmG * mG | | |
| WV- | GCAUCCTTGGCGGTCUUGGU | mG * mCmAmUmC * C * T * T * G * G * C * G * | XOOOXXXXXXXXXXXOOOX | 1111 |
| 1852 | | G * T * C * mUmUmGmG * mU | | |
| WV- | CUGGCCTGCTGGGCCACCUG | mC * mUmGmGmC * C * T * G * C * T * G * G * | XOOOXXXXXXXXXXXOOOX | 1112 |
| 1853 | | G * C * C * mAmCmCmU * mG | | |
| WV- | GGCGGTCTTGGTGGCGUGCU | mG * mGmCmGmG * T * C * T * T * G * G * T * | XOOOXXXXXXXXXXXOOOX | 1113 |
| 1854 | | G * G * C * mGmUmGmC * mU | | |
| WV- | UGGCGGTCTTGGTGGCGUGC | mU * mGmGmCmG * G * T * C * T * T * G * G * | XOOOXXXXXXXXXXXOOOX | 1114 |
| 1855 | | T * G * G * mCmGmUmG * mC | | |
| WV- | CUUGGCGGTCTTGGTGGCGU | mC * mUmUmGmG * C * G * G * T * C * T * T * | XOOOXXXXXXXXXXXOOOX | 1115 |
| 1856 | | G * G * T * mGmGmCmG * mU | | |
| WV- | CCUUGGCGGTCTTGGUGGCG | mC * mCmUmUmG * G * C * G * G * T * C * T * | XOOOXXXXXXXXXXXOOOX | 1116 |
| 1857 | | T * G * G * mUmGmGmC * mG | | |
| WV- | GGCAGAGGCCAGGAGCGCCA | mG * mGmCmAmG * A * G * G * C * C * A * G * | XOOOXXXXXXXXXXXOOOX | 1117 |
| 1858 | | G * A * G * mCmGmCmC * mA | | |
| WV- | GGGAGGCATCCTCGGCCUCU | mG * mGmGmAmG * G * C * A * T * C * C * T * | XOOOXXXXXXXXXXXOOOX | 1118 |
| 1859 | | C * G * G * mCmCmUmC * mU | | |
| WV- | AAGGGAGGCATCCTCGGCCU | mA * mAmGmGmG * A * G * G * C * A * T * C * | XOOOXXXXXXXXXXXOOOX | 1119 |
| 1860 | | C * T * C * mGmGmCmC * mU | | |
| WV- | GAAGGGAGGCATCCTCGGCC | mG * mAmAmGmG * G * A * G * G * C * A * T * | XOOOXXXXXXXXXXXOOOX | 1120 |
| 1861 | | C * C * T * mCmGmGmC * mC | | |
| WV- | GCGGUCTTGGTGGCGUGCUU | mG * mCmGmGmU * C * T * T * G * G * T * G * | XOOOXXXXXXXXXXXOOOX | 1121 |
| 1862 | | G * C * G * mUmGmCmU * mU | | |
| WV- | GUCUCAGGCAGCCACGGCUG | mG * mUmCmUmC * A * G * G * C * A * G * C * | XOOOXXXXXXXXXXXOOOX | 1122 |
| 1863 | | C * A * C * mGmGmCmU * mG | | |
| WV- | UGCAUCCTTGGCGGTCUUGG | mU * mGmCmAmU * C * C * T * T * G * G * C * | XOOOXXXXXXXXXXXOOOX | 1123 |
| 1864 | | G * G * T * mCmUmUmG * mG | | |
| WV- | CUGCUGGGCCACCTGGGACU | mC * mUmGmCmU * G * G * G * C * C * A * C * | XOOOXXXXXXXXXXXOOOX | 1124 |
| 1865 | | C * T * G * mGmGmAmC * mU | | |
| WV- | UGAAGCCATCGGTCACCCAG | mU * mGmAmAmG * C * C * A * T * C * G * G * | XOOOXXXXXXXXXXXOOOX | 1125 |
| 1866 | | T * C * A * mCmCmCmA * mG | | |
| WV- | CUUGUCCTTAACGGTGCUCC | mC * mUmUmGmU * C * C * T * T * A * A * C * | XOOOXXXXXXXXXXXOOOX | 1126 |
| 1867 | | G * G * T * mGmCmUmC * mC | | |
| WV- | UGUCCAGCTTTATTGGGAGG | mU * mGmUmCmC * A * G * C * T * T * T * A * | XOOOXXXXXXXXXXXOOOX | 1127 |
| 1868 | | T * T * G * mGmGmAmG * mG | | |
| WV- | CUGCATGGCACCTCTGUUCC | mC * mUmGmCmA * T * G * G * C * A * C * C * | XOOOXXXXXXXXXXXOOOX | 1128 |
| 1869 | | T * C * T * mGmUmUmC * mC | | |
| WV- | UGCUCAGTGCATCCTUGGCG | mU * mGmCm UmC * A * G * T * G * C * A * T * | XOOOXXXXXXXXXXXOOOX | 1129 |
| 1870 | | C * C * T * mUmGmGmC * mG | | |
| WV- | CCUGGGACTCCTGCACGCUG | mC * mCmUmGmG * G * A * C * T * C * C * T * | XOOOXXXXXXXXXXXOOOX | 1130 |
| 1871 | | G * C * A * mCmGmCmU * mG | | |
| WV- | UCGGUCACCCAGCCCCUGGC | mU * mCmGmGmU * C * A * C * C * C * A * G * | XOOOXXXXXXXXXXXOOOX | 1131 |
| 1872 | | C * C * C * mCmUmGmG * mC | | |
| WV- | AUCGGTCACCCAGCCCCUGG | mA * mUmCmGmG * T * C * A * C * C * C * A * | XOOOXXXXXXXXXXXOOOX | 1132 |
| 1873 | | G * C * C * mCmCmUmG * mG | | |
| WV- | CAUCGGTCACCCAGCCCCUG | mC * mAmUmCmG * G * T * C * A * C * C * C * | XOOOXXXXXXXXXXXOOOX | 1133 |
| 1874 | | A * G * C * mCmCmCmU * mG | | |
| WV- | CCAUCGGTCACCCAGCCCCU | mC * mCmAmUmC * G * G * T * C * A * C * C * | XOOOXXXXXXXXXXXOOOX | 1134 |
| 1875 | | C * A * G * mCmCmCmC * mU | | |
| WV- | GCCAUCGGTCACCCAGCCCC | mG * mCmCmAmU * C * G * G * T * C * A * C * | XOOOXXXXXXXXXXXOOOX | 1135 |
| 1876 | | C * C * A * mGmCmCmC * mC | | |
| WV- | AGCCATCGGTCACCCAGCCC | mA * mGmCmCmA * T * C * G * G * T * C * A * | XOOOXXXXXXXXXXXOOOX | 1136 |
| 1877 | | C * C * C * mAmGmCmC * mC | | |
| WV- | UCCAGCTTTATTGGGAGGCC | mU * mCmCmAmG * C * T * T * T * A * T * T * | XOOOXXXXXXXXXXXOOOX | 1137 |
| 1878 | | G * G * G * mAmGmGmC * mC | | |
| WV- | CACGCTGCTCAGTGCAUCCU | mC * mAmCmGmC * T * G * C * T * C * A * G * | XOOOXXXXXXXXXXXOOOX | 1138 |
| 1879 | | T * G * C * mAmUmCmC * mU | | |
| WV- | CUCCUGCACGCTGCTCAGUG | mC * mUmCmCmU * G * C * A * C * G * C * T * | XOOOXXXXXXXXXXXOOOX | 1139 |
| 1880 | | G * C * T * mCmAmGmU * mG | | |
| WV- | GGGACTCCTGCACGCUGCUC | mG * mGmGmAmC * T * C * C * T * G * C * A * | XOOOXXXXXXXXXXXOOOX | 1140 |
| 1881 | | C * G * C * mUmGmCmU * mC | | |
| WV- | UGGGACTCCTGCACGCUGCU | mU * mGmGmGmA * C * T * C * C * T * G * C * | XOOOXXXXXXXXXXXOOOX | 1141 |
| 1882 | | A * C * G * mCmUmGmC * mU | | |
| WV- | AGGUCTCAGGCAGCCACGGC | mA * mGmGmUmC * T * C * A * G * G * C * A * | XOOOXXXXXXXXXXXOOOX | 1142 |
| 1883 | | G * C * C * mAmCmGmG * mC | | |
| WV- | GAGGUCTCAGGCAGCCACGG | mG * mAmGmGmU * C * T * C * A * G * G * C * | XOOOXXXXXXXXXXXOOOX | 1143 |
| 1884 | | A * G * C * mCmAmCmG * mG | | |
| WV- | UGAGGTCTCAGGCAGCCACG | mU * mGmAmGmG * T * C * T * C * A * G * G * | XOOOXXXXXXXXXXXOOOX | 1144 |
| 1885 | | C * A * G * mCmCmAmC * mG | | |
| WV- | CCUGGAGATTGCAGGACCCA | mC * mCmUmGmG * A * G * A * T * T * G * C * | XOOOXXXXXXXXXXXOOOX | 1145 |
| 1886 | | A * G * G * mAmCmCmC * mA | | |
| WV- | GCCCUGGAGATTGCAGGACC | mG * mCmCmCmU * G * G * A * G * A * T * T * | XOOOXXXXXXXXXXXOOOX | 1146 |
| 1887 | | G * C * A * mGmGmAmC * mC | | |
| WV- | CGUGCTTCATGTAACCCUGC | mC * mGmUmGmC * T * T * C * A * T * G * T * | XOOOXXXXXXXXXXXOOOX | 1147 |
| 1888 | | A * A * C * mCmCmUmG * mC | | |
| WV- | UGGUCTGACCTCAGGGUCCA | mU * mGmGmUmC * T * G * A * C * C * T * C * | XOOOXXXXXXXXXXXOOOX | 1148 |
| 1889 | | A * G * G * mGmUmCmC * mA | | |
| WV- | AAGUUGGTCTGACCTCAGGG | mA * mAmGmUmU * G * G * T * C * T * G * A * | XOOOXXXXXXXXXXXOOOX | 1149 |
| 1890 | | C * C * T * mCmAmGmG * mG | | |
| WV- | CCACGGCTGAAGTTGGUCUG | mC * mCmAmCmG * G * C * T * G * A * A * G * | XOOOXXXXXXXXXXXOOOX | 1150 |
| 1891 | | T * T * G * mGmUmCmU * mG | | |
| WV- | AGCCACGGCTGAAGTUGGUC | mA * mGmCmCmA * C * G * G * C * T * G * A * | XOOOXXXXXXXXXXXOOOX | 1151 |
| 1892 | | A * G * T * mUmGmGmU * mC | | |
| WV- | GUCUUGGTGGCGTGCUUCAU | mG * mUmCmUmU * G * G * T * G * G * C * G * | XOOOXXXXXXXXXXXOOOX | 1152 |
| 1893 | | T * G * C * mUmUmCmA * mU | | |
| WV- | GCGUGCTTCATGTAACCCUG | mG * mCmGmUmG * C * T * T * C * A * T * G * | XOOOXXXXXXXXXXXOOOX | 1153 |
| 1894 | | T * A * A * mCmCmCmU * mG | | |
| WV- | GCUGAAGTTGGTCTGACCUC | mG * mCmUmGmA * A * G * T * T * G * G * T * | XOOOXXXXXXXXXXXOOOX | 1154 |
| 1895 | | C * T * G * mAmCmCmU * mC | | |
| WV- | GGGCCTCCCAAGGCAAACCC | mG * mGmGmCmC * T * C * C * C * A * A * G * | XOOOXXXXXXXXXXXOOOX | 1155 |
| 1896 | | G * C * A * mAmAmCmC * mC | | |
| WV- | AACCUTAGCTGGGTCUGCCA | mA * mAmCmCmU * T * A * G * C * T * G * G * | XOOOXXXXXXXXXXXOOOX | 1156 |
| 1897 | | G * T * C * mUmGmCmC * mA | | |
| WV- | UUGUCCAGCTTTATTGGGAG | mU * mU * mG * mU * mC * C * A * G * C * T * | XXXXXXXXXXXXXXXXXXX | 1157 |
| 2115 | | T * T * A * T * T * mG * mG * mG * mA * mG | | |
| WV- | CUUGUCCAGCTTTATUGGGA | mC * mU * mU * mG * mU * C * C * A * G * C * | XXXXXXXXXXXXXXXXXXX | 1158 |
| 2116 | | T * T * T * A * T * mU * mG * mG * mG * mA | | |
| WV- | UCUUGTCCAGCTTTAUUGGG | mU * mC * mU * mU * mG * T * C * C * A * G * | XXXXXXXXXXXXXXXXXXX | 1159 |
| 2117 | | C * T * T * T * A * mU * mU * mG * mG * mG | | |
| WV- | UUCUUGTCCAGCTTTAUUGG | mU * mU * mC * mU * mU * G * T * C * C * A * | XXXXXXXXXXXXXXXXXXX | 1160 |
| 2118 | | G * C * T * T * T * mA * mU * mU * mG * mG | | |
| WV- | CUUCUTGTCCAGCTTUAUUG | mC * mU * mU * mC * mU * T * G * T * C * C * | XXXXXXXXXXXXXXXXXXX | 1161 |
| 2119 | | A * G * C * T * T * mU * mA * mU * mU * mG | | |
| WV- | GCUUCTTGTCCAGCTUUAUU | mG * mC * mU * mU * mC * T * T * G * T * C * C | XXXXXXXXXXXXXXXXXXX | 1162 |
| 2120 | | * A * G * C * T * mU * mU * mA * mU * mU | | |
| WV- | AGCUUCTTGTCCAGCUUUAU | mA * mG * mC * mU * mU * C * T * T * G * T * | XXXXXXXXXXXXXXXXXXX | 1163 |
| 2121 | | C * C * A * G * C * mU * mU * mU * mA * mU | | |
| WV- | CAGCUTCTTGTCCAGCUUUA | mC * mA * mG * mC * mU * T * C * T * T * G * T | XXXXXXXXXXXXXXXXXXX | 1164 |
| 2122 | | * C * C * A * G * mC * mU * mU * mU * mA | | |
| WV- | GCAGCTTCTTGTCCAGCUUU | mG * mC * mA * mG * mC * T * T * C * T * T * G | XXXXXXXXXXXXXXXXXXX | 1165 |
| 2123 | | * T * C * C * A * mG * mC * mU * mU * mU | | |
| WV- | AGCAGCTTCTTGTCCAGCUU | mA * mG * mC * mA * mG * C * T * T * C * T * T | XXXXXXXXXXXXXXXXXXX | 1166 |
| 2124 | | * G * T * C * C * mA * mG * mC * mU * mU | | |
| WV- | UAGCAGCTTCTTGTCCAGCU | mU * mA * mG * mC * mA * G * C * T * T * C * T | XXXXXXXXXXXXXXXXXXX | 1167 |
| 2125 | | * T * G * T * C * mC * mA * mG * mC * mU | | |
| WV- | AUAGCAGCTTCTTGTCCAGC | mA * mU * mA * mG * mC * A * G * C * T * T * | XXXXXXXXXXXXXXXXXXX | 1168 |
| 2126 | | C * T * T * G * T * mC * mC * mA * mG * mC | | |
| WV- | CAUAGCAGCTTCTTGUCCAG | mC * mA * mU * mA * mG * C * A * G * C * T * | XXXXXXXXXXXXXXXXXXX | 1169 |
| 2127 | | T * C * T * T * G * mU * mC * mC * mA * mG | | |
| WV- | UUGUCCAGCTTTATTGGGAG | mU * mUmGmUmC * C * A * G * C * T * T * T * | XOOOXXXXXXXXXXXOOOX | 1170 |
| 2128 | | A * T * T * mGmGmGmA * mG | | |
| WV- | CUUGUCCAGCTTTATUGGGA | mC * mUmUmGmU * C * C * A * G * C * T * T * | XOOOXXXXXXXXXXXOOOX | 1171 |
| 2129 | | T * A * T * mUmGmGmG * mA | | |
| WV- | UCUUGTCCAGCTTTAUUGGG | mU * mCmUmUmG * T * C * C * A * G * C * T * | XOOOXXXXXXXXXXXOOOX | 1172 |
| 2130 | | T * T * A * mUmUmGmG * mG | | |
| WV- | UUCUUGTCCAGCTTTAUUGG | mU * mUmCmUmU * G * T * C * C * A * G * C * | XOOOXXXXXXXXXXXOOOX | 1173 |
| 2131 | | T * T * T * mAmUmUmG * mG | | |
| WV- | CUUCUTGTCCAGCTTUAUUG | mC * mUmUmCmU * T * G * T * C * C * A * G * | XOOOXXXXXXXXXXXOOOX | 1174 |
| 2132 | | C * T * T * mUmAmUmU * mG | | |
| WV- | GCUUCTTGTCCAGCTUUAUU | mG * mCmUmUmC * T * T * G * T * C * C * A * | XOOOXXXXXXXXXXXOOOX | 1175 |
| 2133 | | G * C * T * mUmUmAmU * mU | | |
| WV- | AGCUUCTTGTCCAGCUUUAU | mA * mGmCmUmU * C * T * T * G * T * C * C * | XOOOXXXXXXXXXXXOOOX | 1176 |
| 2134 | | A * G * C * mUmUmUmA * mU | | |
| WV- | CAGCUTCTTGTCCAGCUUUA | mC * mAmGmCmU * T * C * T * T * G * T * C * | XOOOXXXXXXXXXXXOOOX | 1177 |
| 2135 | | C * A * G * mCmUmUmU * mA | | |
| WV- | GCAGCTTCTTGTCCAGCUUU | mG * mCmAmGmC * T * T * C * T * T * G * T * C | XOOOXXXXXXXXXXXOOOX | 1178 |
| 2136 | | * C * A * mGmCmUmU * mU | | |
| WV- | AGCAGCTTCTTGTCCAGCUU | mA * mGmCmAmG * C * T * T * C * T * T * G * T | XOOOXXXXXXXXXXXOOOX | 1179 |
| 2137 | | * C * C * mAmGmCmU * mU | | |
| WV- | UAGCAGCTTCTTGTCCAGCU | mU * mAmGmCmA * G * C * T * T * C * T * T * | XOOOXXXXXXXXXXXOOOX | 1180 |
| 2138 | | G * T * C * mCmAmGmC * mU | | |
| WV- | AUAGCAGCTTCTTGTCCAGC | mA * mUmAmGmC * A * G * C * T * T * C * T * | XOOOXXXXXXXXXXXOOOX | 1181 |
| 2139 | | T * G * T * mCmCmAmG * mC | | |
| WV- | CAUAGCAGCTTCTTGUCCAG | mC * mAmUmAmG * C * A * G * C * T * T * C * | XOOOXXXXXXXXXXXOOOX | 1182 |
| 2140 | | T * T * G * mUmCmCmA * mG | | |
| WV- | AGCTTCTTGTCCAGCTTTAT | Aeo * Geo * m5Ceo * Teo * Teo * C * T * T * G * | XXXXXXXXXXXXXXXXXXX | 1183 |
| 2141 | | T * C * C * A * G * C * Teo * Teo * Teo * Aeo * | | |
| | | Teo | | |
| WV- | UGUCCAGCTTTATTGGGAGG | mU * SmGmUmCmC * SA * SG * SC * ST * ST * | SOOOSSSSSSSSRSSOOOS | 1184 |
| 2549 | | ST * SA * ST * RT * SG * SmGmGmAmG * SmG | | |
| WV- | UGUCCAGCTTTATTGGGAGG | mU * SmGmUmCmC * SA * SG * SC * ST * ST * | SOOOSSSSSSSRSSSOOOS | 1185 |
| 2550 | | ST * SA * RT * ST * SG * SmGmGmAmG * SmG | | |
| WV- | UGUCCAGCTTTATTGGGAGG | mU * SmGmUmCmC * SA * SG * SC * ST * ST * | SOOOSSSSSSRSSSSOOOS | 1186 |
| 2551 | | ST * RA * ST * ST * SG * SmGmGmAmG * SmG | | |
| WV- | UGUCCAGCTTTATTGGGAGG | mU * SmGmUmCmCmA * SG * SC * ST * ST * ST | SOOOOSSSSSSSRSSSOOS | 1187 |
| 2552 | | * SA * ST * RT * SG * SG * SmGmAmG * SmG | | |
| WV- | UGUCCAGCTTTATTGGGAGG | mU * SmGmUmCmC * SA * SG * SC * ST * ST * | SOOOSSSSSSSSSSRSSOS | 1188 |
| 2553 | | ST * SA * ST * ST * SG * RG * SG * SmAmG * | | |
| | | SmG | | |
| WV- | UGUCCAGCTTTATTGGGAGG | mU * SmGmUmCmCmA * SG * SC * ST * ST * ST | SOOOOSSSSSSSSSRSSSS | 1189 |
| 2554 | | * SA * ST * ST * SG * RG * SG * SA * SmG * SmG | | |
| WV- | UGUCCAGCTTTATTGGGAGG | mU * mGmUmCmC * A * G * C * T * T * T * A * | XOOOXXXXXXXXXXXXXOX | 1190 |
| 2677 | | T * T * G * G * G * mAmG * mG | | |
| WV- | UGUCCAGCTTTATTGGGAGG | mU * mGmUmCmCmA * G * C * T * T * T * A * | XOOOXXXXXXXXXXXXXX | 1191 |
| 2678 | | T * T * G * G * G * A * mG * mG | | |
| WV- | GGAGCAGCTGCCTCTAGGGA | mG * mG * mA * mG * mc * A * G * C * T * G * | XXXXXXXXXXXXXXXXXXX | 1192 |
| 1391 | | C * C * T * C * T * mA * mG * mG * mG * mA | | |
| WV- | UGGAGCAGCTGCCTCUAGGG | mU * mG * mG * mA * mG * C * A * G * C * T * | XXXXXXXXXXXXXXXXXXX | 1193 |
| 1392 | | G * C * C * T * C * mU * mA * mG * mG * mG | | |
| WV- | UGUUCCTGGAGCAGCUGCCU | mU * mG * mU * mU * mC * C * T * G * G * A * | XXXXXXXXXXXXXXXXXXX | 1194 |
| 1393 | | G * C * A * G * C * mU * mG * mC * mC * mU | | |
| WV- | UCCUUGGCGGTCTTGGUGGC | mU * mC * mC * mU * mU * G * G * C * G * G | XXXXXXXXXXXXXXXXXXX | 1195 |
| 1394 | | * T * C * T * T * G * mG * mU * mG * mG * mC | | |
| WV- | AUCCUTGGCGGTCTTGGUGG | mA * mU * mC * mC * mU * T * G * G * C * G * | XXXXXXXXXXXXXXXXXXX | 1196 |
| 1395 | | G * T * C * T * T * mG * mG * mU * mG * mG | | |
| WV- | CAUCCTTGGCGGTCTUGGUG | mC * mA * mU * mC * mC * T * T * G * G * C * | XXXXXXXXXXXXXXXXXXX | 1197 |
| 1396 | | G * G * T * C * T * mU * mG * mG * mU * mG | | |
| WV- | GCAUCCTTGGCGGTCUUGGU | mG * mC * mA * mU * mC * C * T * T * G * G * | XXXXXXXXXXXXXXXXXXX | 1198 |
| 1397 | | C * G * G * T * C * mU * mU * mG * mG * mU | | |
| WV- | CUGGCCTGCTGGGCCACCUG | mC * mU * mG * mG * mC * C * T * G * C * T * | XXXXXXXXXXXXXXXXXXX | 1199 |
| 1398 | | G * G * G * C * C * mA * mC * mC * mU * mG | | |
| WV- | GGCGGTCTTGGTGGCGUGCU | mG * mG * mC * mG * mG * T * C * T * T * G * | XXXXXXXXXXXXXXXXXXX | 1200 |
| 1399 | | G * T * G * G * C * mG * mU * mG * mC * mU | | |
| WV- | UGGCGGTCTTGGTGGCGUGC | mU * mG * mG * mC * mG * G * T * C * T * T * | XXXXXXXXXXXXXXXXXXX | 1201 |
| 1400 | | G * G * T * G * G * mC * mG * mU * mG * mC | | |
| WV- | UUGGCGGTCTTGGTGGCGUG | mU * mU * mG * mG * mC * G * G * T * C * T * | XXXXXXXXXXXXXXXXXXX | 1202 |
| 1401 | | T * G * G * T * G * mG * mC * mG * mU * mG | | |
| WV- | CUUGGCGGTCTTGGTGGCGU | mC * mU * mU * mG * mG * C * G * G * T * C * | XXXXXXXXXXXXXXXXXXX | 1203 |
| 1402 | | T * T * G * G * T * mG * mG * mC * mG * mU | | |
| WV- | CCUUGGCGGTCTTGGUGGCG | mC * mC * mU * mU * mG * G * C * G * G * T * | XXXXXXXXXXXXXXXXXXX | 1204 |
| 1403 | | C * T * T * G * G * mU * mG * mG * mC * mG | | |
| WV- | GCCCCTGGCCTGCTGGGCCA | mG * mC * mC * mC * mC * T * G * G * C * C * | XXXXXXXXXXXXXXXXXXX | 1205 |
| 1404 | | T * G * C * T * G * mG * mG * mC * mC * mA | | |
| WV- | GGCAGAGGCCAGGAGCGCCA | mG * mG * mC * mA * mG * A * G * G * C * C * | XXXXXXXXXXXXXXXXXXX | 1206 |
| 1405 | | A * G * G * A * G * mC * mG * mC * mC * mA | | |
| WV- | GAGGCATCCTCGGCCUCUGA | mG * mA * mG * mG * mC * A * T * C * C * T * | XXXXXXXXXXXXXXXXXXX | 1207 |
| 1406 | | C * G * G * C * C * mU * mC * mU * mG * mA | | |
| WV- | GGAGGCATCCTCGGCCUCUG | mG * mG * mA * mG * mG * C * A * T * C * C * | XXXXXXXXXXXXXXXXXXX | 1208 |
| 1407 | | T * C * G * G * C * mC * mU * mC * mU * mG | | |
| WV- | GGGAGGCATCCTCGGCCUCU | mG * mG * mG * mA * mG * G * C * A * T * C * | XXXXXXXXXXXXXXXXXXX | 1209 |
| 1408 | | C * T * C * G * G * mC * mC * mU * mC * mU | | |
| WV- | AGGGAGGCATCCTCGGCCUC | mA * mG * mG * mG * mA * G * G * C * A * T * | XXXXXXXXXXXXXXXXXXX | 1210 |
| 1409 | | C * C * T * C * G * mG * mC * mC * mU * mC | | |
| WV- | AAGGGAGGCATCCTCGGCCU | mA * mA * mG * mG * mG * A * G * G * C * A | XXXXXXXXXXXXXXXXXXX | 1211 |
| 1410 | | * T * C * C * T * C * mG * mG * mC * mC * mU | | |
| WV- | GAAGGGAGGCATCCTCGGCC | mG * mA * mA * mG * mG * G * A * G * G * C | XXXXXXXXXXXXXXXXXXX | 1212 |
| 1411 | | * A * T * C * C * T * mC * mG * mG * mC * mC | | |
| WV- | GGUCUTGGTGGCGTGCUUCA | mG * mG * mU * mC * mU * T * G * G * T * G * | XXXXXXXXXXXXXXXXXXX | 1213 |
| 1412 | | G * C * G * T * G * mC * mU * mU * mC * mA | | |
| WV- | GCGGUCTTGGTGGCGUGCUU | mG * mC * mG * mG * mU * C * T * T * G * G * | XXXXXXXXXXXXXXXXXXX | 1214 |
| 1413 | | T * G * G * C * G * mU * mG * mC * mU * mU | | |
| WV- | GUCUCAGGCAGCCACGGCUG | mG * mU * mC * mU * mC * A * G * G * C * A * | XXXXXXXXXXXXXXXXXXX | 1215 |
| 1414 | | G * C * C * A * C * mG * mG * mC * mU * mG | | |
| WV- | AGGCCAGCATGCCTGGAGGG | mA * mG * mG * mC * mC * A * G * C * A * T * | XXXXXXXXXXXXXXXXXXX | 1216 |
| 1415 | | G * C * C * T * G * mG * mA * mG * mG * mG | | |
| WV- | UGCAUCCTTGGCGGTCUUGG | mU * mG * mC * mA * mU * C * C * T * T * G * | XXXXXXXXXXXXXXXXXXX | 1217 |
| 1416 | | G * C * G * G * T * mC * mU * mU * mG * mG | | |
| WV- | GUGCATCCTTGGCGGUCUUG | mG * mU * mG * mC * mA * T * C * C * T * T * | XXXXXXXXXXXXXXXXXXX | 1218 |
| 1417 | | G * G * C * G * G * mU * mC * mU * mU * mG | | |
| WV- | CUGCUGGGCCACCTGGGACU | mC * mU * mG * mC * mU * G * G * G * C * C * | XXXXXXXXXXXXXXXXXXX | 1219 |
| 1418 | | A * C * C * T * G * mG * mG * mA * mC * mU | | |
| WV- | UGAAGCCATCGGTCACCCAG | mU * mG * mA * mA * mG * C * C * A * T * C * | XXXXXXXXXXXXXXXXXXX | 1220 |
| 1419 | | G * G * T * C * A * mC * mC * mC * mA * mG | | |
| WV- | CUGAAGCCATCGGTCACCCA | mC * mU * mG * mA * mA * G * C * C * A * T * | XXXXXXXXXXXXXXXXXXX | 1221 |
| 1420 | | C * G * G * T * C * mA * mC * mC * mC * mA | | |
| WV- | CUUGUCCTTAACGGTGCUCC | mC * mU * mU * mG * mU * C * C * T * T * A * | XXXXXXXXXXXXXXXXXXX | 1222 |
| 1421 | | A * C * G * G * T * mG * mC * mU * mC * mC | | |
| WV- | UGUCCAGCTTTATTGGGAGG | mU * mG * mU * mC * mC * A * G * C * T * T * | XXXXXXXXXXXXXXXXXXX | 1223 |
| 1422 | | T * A * T * T * G * mG * mG * mA * mG * mG | | |
| WV- | UGCCUCTAGGGATGAACUGA | mU * mG * mC * mC * mU * C * T * A * G * G * | XXXXXXXXXXXXXXXXXXX | 1224 |
| 1423 | | G * A * T * G * A * mA * mC * mU * mG * mA | | |
| WV- | CUGCATGGCACCTCTGUUCC | mC * mU * mG * mC * mA * T * G * G * C * A * | XXXXXXXXXXXXXXXXXXX | 1225 |
| 1424 | | C * C * T * C * T * mG * mU * mU * mC * mC | | |
| WV- | GGGCUGCATGGCACCUCUGU | mG * mG * mG * mC * mU * G * C * A * T * G * | XXXXXXXXXXXXXXXXXXX | 1226 |
| 1425 | | G * C * A * C * C * mU * mC * mU * mG * mU | | |
| WV- | CUCUGAAGCTCGGGCAGAGG | mC * mU * mC * mU * mG * A * A * G * C * T * | XXXXXXXXXXXXXXXXXXX | 1227 |
| 1426 | | C * G * G * G * C * mA * mG * mA * mG * mG | | |
| WV- | CCUCUGAAGCTCGGGCAGAG | mC * mC * mU * mC * mU * G * A * A * G * C * | XXXXXXXXXXXXXXXXXXX | 1228 |
| 1427 | | T * C * G * G * G * mC * mA * mG * mA * mG | | |
| WV- | GCCUCTGAAGCTCGGGCAGA | mG * mC * mC * mU * mC * T * G * A * A * G * | XXXXXXXXXXXXXXXXXXX | 1229 |
| 1428 | | C * T * C * G * G * mG * mC * mA * mG * mA | | |
| WV- | CGGCCTCTGAAGCTCGGGCA | mC * mG * mG * mC * mC * T * C * T * G * A * | XXXXXXXXXXXXXXXXXXX | 1230 |
| 1429 | | A * G * C * T * C * mG * mG * mG * mC * mA | | |
| WV- | UCGGCCTCTGAAGCTCGGGC | mU * mC * mG * mG * mC * C * T * C * T * G * | XXXXXXXXXXXXXXXXXXX | 1231 |
| 1430 | | A * A * G * C * T * mC * mG * mG * mG * mC | | |
| WV- | CUCGGCCTCTGAAGCUCGGG | mC * mU * mC * mG * mG * C * C * T * C * T * | XXXXXXXXXXXXXXXXXXX | 1232 |
| 1431 | | G * A * A * G * C * mU * mC * mG * mG * mG | | |
| WV- | CCUCGGCCTCTGAAGCUCGG | mC * mC * mU * mC * mG * G * C * C * T * C * | XXXXXXXXXXXXXXXXXXX | 1233 |
| 1432 | | T * G * A * A * G * mC * mU * mC * mG * mG | | |
| WV- | UCCUCGGCCTCTGAAGCUCG | mU * mC * mC * mU * mC * G * G * C * C * T * | XXXXXXXXXXXXXXXXXXX | 1234 |
| 1433 | | C * T * G * A * A * mG * mC * mU * mC * mG | | |
| WV- | UGCUCAGTGCATCCTUGGCG | mU * mG * mC * mU * mC * A * G * T * G * C * | XXXXXXXXXXXXXXXXXXX | 1235 |
| 1434 | | A * T * C * C * T * mU * mG * mG * mC * mG | | |
| WV- | CCUGGGACTCCTGCACGCUG | mC * mC * mU * mG * mG * G * A * C * T * C * | XXXXXXXXXXXXXXXXXXX | 1236 |
| 1435 | | C * T * G * C * A * mC * mG * mC * mU * mG | | |
| WV- | CCACCTGGGACTCCTGCACG | mC * mC * mA * mC * mC * T * G * G * G * A * | XXXXXXXXXXXXXXXXXXX | 1237 |
| 1436 | | C * T * C * C * T * mG * mC * mA * mC * mG | | |
| WV- | UCGGUCACCCAGCCCCUGGC | mU * mC * mG * mG * mU * C * A * C * C * C * | XXXXXXXXXXXXXXXXXXX | 1238 |
| 1437 | | A * G * C * C * C * mC * mU * mG * mG * mC | | |
| WV- | AUCGGTCACCCAGCCCCUGG | mA * mU * mC * mG * mG * T * C * A * C * C * | XXXXXXXXXXXXXXXXXXX | 1239 |
| 1438 | | C * A * G * C * C * mC * mC * mU * mG * mG | | |
| WV- | CAUCGGTCACCCAGCCCCUG | mC * mA * mU * mC * mG * G * T * C * A * C * | XXXXXXXXXXXXXXXXXXX | 1240 |
| 1439 | | C * C * A * G * C * mC * mC * mC * mU * mG | | |
| WV- | CCAUCGGTCACCCAGCCCCU | mC * mC * mA * mU * mC * G * G * T * C * A * | XXXXXXXXXXXXXXXXXXX | 1241 |
| 1440 | | C * C * C * A * G * mC * mC * mC * mC * mU | | |
| WV- | GCCAUCGGTCACCCAGCCCC | mG * mC * mC * mA * mU * C * G * G * T * C * | XXXXXXXXXXXXXXXXXXX | 1242 |
| 1441 | | A * C * C * C * A * mG * mC * mC * mC * mC | | |
| WV- | AGCCATCGGTCACCCAGCCC | mA * mG * mC * mC * mA * T * C * G * G * T * | XXXXXXXXXXXXXXXXXXX | 1243 |
| 1442 | | C * A * C * C * C * mA * mG * mC * mC * mC | | |
| WV- | UCCAGCTTTATTGGGAGGCC | mU * mC * mC * mA * mG * C * T * T * T * A * | XXXXXXXXXXXXXXXXXXX | 1244 |
| 1443 | | T * T * G * G * G * mA * mG * mG * mC * mC | | |
| WV- | CAUCCTCGGCCTCTGAAGCU | mC * mA * mU * mC * mC * T * C * G * G * C * | XXXXXXXXXXXXXXXXXXX | 1245 |
| 1444 | | C * T * C * T * G * mA * mA * mG * mC * mU | | |
| WV- | AGGCATCCTCGGCCTCUGAA | mA * mG * mG * mC * mA * T * C * C * T * C * | XXXXXXXXXXXXXXXXXXX | 1246 |
| 1445 | | G * G * C * C * T * mC * mU * mG * mA * mA | | |
| WV- | UCUUGGTGGCGTGCTUCAUG | mU * mC * mU * mU * mG * G * T * G * G * C | XXXXXXXXXXXXXXXXXXX | 1247 |
| 1446 | | * G * T * G * C * T * mU * mC * mA * mU * mG | | |
| WV- | CACGCTGCTCAGTGCAUCCU | mC * mA * mC * mG * mC * T * G * C * T * C * | XXXXXXXXXXXXXXXXXXX | 1248 |
| 1447 | | A * G * T * G * C * mA * mU * mC * mC * mU | | |
| WV- | CUCCUGCACGCTGCTCAGUG | mC * mU * mC * mC * mU * G * C * A * C * G * | XXXXXXXXXXXXXXXXXXX | 1249 |
| 1448 | | C * T * G * C * T * mC * mA * mG * mU * mG | | |
| WV- | GGACUCCTGCACGCTGCUCA | mG * mG * mA * mC * mU * C * C * T * G * C * | XXXXXXXXXXXXXXXXXXX | 1250 |
| 1449 | | A * C * G * C * T * mG * mC * mU * mC * mA | | |
| WV- | GGGACTCCTGCACGCUGCUC | mG * mG * mG * mA * mC * T * C * C * T * G * | XXXXXXXXXXXXXXXXXXX | 1251 |
| 1450 | | C * A * C * G * C * mU * mG * mC * mU * mC | | |
| WV- | UGGGACTCCTGCACGCUGCU | mU * mG * mG * mG * mA * C * T * C * C * T * | XXXXXXXXXXXXXXXXXXX | 1252 |
| 1451 | | G * C * A * C * G * mC * mU * mG * mC * mU | | |
| WV- | AGGUCTCAGGCAGCCACGGC | mA * mG * mG * mU * mC * T * C * A * G * G * | XXXXXXXXXXXXXXXXXXX | 1253 |
| 1452 | | C * A * G * C * C * mA * mC * mG * mG * mC | | |
| WV- | GAGGUCTCAGGCAGCCACGG | mG * mA * mG * mG * mU * C * T * C * A * G * | XXXXXXXXXXXXXXXXXXX | 1254 |
| 1453 | | G * C * A * G * C * mC * mA * mC * mG * mG | | |
| WV- | UGAGGTCTCAGGCAGCCACG | mU * mG * mA * mG * mG * T * C * T * C * A * | XXXXXXXXXXXXXXXXXXX | 1255 |
| 1454 | | G * G * C * A * G * mC * mC * mA * mC * mG | | |
| WV- | CCUGGAGATTGCAGGACCCA | mC * mC * mU * mG * mG * A * G * A * T * T * | XXXXXXXXXXXXXXXXXXX | 1256 |
| 1455 | | G * C * A * G * G * mA * mC * mC * mC * mA | | |
| WV- | GCCCUGGAGATTGCAGGACC | mG * mC * mC * mC * mU * G * G * A * G * A * | XXXXXXXXXXXXXXXXXXX | 1257 |
| 1456 | | T * T * G * C * A * mG * mG * mA * mC * mC | | |
| WV- | CCAGGAGCGCCAGGAGGGCA | mC * mC * mA * mG * mG * A * G * C * G * C * | XXXXXXXXXXXXXXXXXXX | 1258 |
| 1457 | | C * A * G * G * A * mG * mG * mG * mC * mA | | |
| WV- | CGUGCTTCATGTAACCCUGC | mC * mG * mU * mG * mC * T * T * C * A * T * | XXXXXXXXXXXXXXXXXXX | 1259 |
| 1458 | | G * T * A * A * C * mC * mC * mU * mG * mC | | |
| WV- | UGGUCTGACCTCAGGGUCCA | mU * mG * mG * mU * mC * T * G * A * C * C * | XXXXXXXXXXXXXXXXXXX | 1260 |
| 1459 | | T * C * A * G * G * mG * mU * mC * mC * mA | | |
| WV- | UUGGUCTGACCTCAGGGUCC | mU * mU * mG * mG * mU * C * T * G * A * C * | XXXXXXXXXXXXXXXXXXX | 1261 |
| 1460 | | C * T * C * A * G * mG * mG * mU * mC * mC | | |
| WV- | AAGUUGGTCTGACCTCAGGG | mA * mA * mG * mU * mU * G * G * T * C * T * | XXXXXXXXXXXXXXXXXXX | 1262 |
| 1461 | | G * A * C * C * T * mC * mA * mG * mG * mG | | |
| WV- | UGAAGTTGGTCTGACCUCAG | mU * mG * mA * mA * mG * T * T * G * G * T * | XXXXXXXXXXXXXXXXXXX | 1263 |
| 1462 | | C * T * G * A * C * mC * mU * mC * mA * mG | | |
| WV- | CACGGCTGAAGTTGGUCUGA | mC * mA * mC * mG * mG * C * T * G * A * A * | XXXXXXXXXXXXXXXXXXX | 1264 |
| 1463 | | G * T * T * G * G * mU * mC * mU * mG * mA | | |
| WV- | CCACGGCTGAAGTTGGUCUG | mC * mC * mA * mC * mG * G * C * T * G * A * | XXXXXXXXXXXXXXXXXXX | 1265 |
| 1464 | | A * G * T * T * G * mG * mU * mC * mU * mG | | |
| WV- | GCCACGGCTGAAGTTGGUCU | mG * mC * mC * mA * mC * G * G * C * T * G * | XXXXXXXXXXXXXXXXXXX | 1266 |
| 1465 | | A * A * G * T * T * mG * mG * mU * mC * mU | | |
| WV- | AGCCACGGCTGAAGTUGGUC | mA * mG * mC * mC * mA * C * G * G * C * T * | XXXXXXXXXXXXXXXXXXX | 1267 |
| 1466 | | G * A * A * G * T * mU * mG * mG * mU * mC | | |
| WV- | GUCUUGGTGGCGTGCUUCAU | mG * mU * mC * mU * mU * G * G * T * G * G | XXXXXXXXXXXXXXXXXXX | 1268 |
| 1467 | | * C * G * T * G * C * mU * mU * mC * mA * mU | | |
| WV- | CAGUGCATCCTTGGCGGUCU | mC * mA * mG * mU * mG * C * A * T * C * C * | XXXXXXXXXXXXXXXXXXX | 1269 |
| 1468 | | T * T * G * G * C * mG * mG * mU * mC * mU | | |
| WV- | CUGCCTCTAGGGATGAACUG | mC * mU * mG * mC * mC * T * C * T * A * G * | XXXXXXXXXXXXXXXXXXX | 1270 |
| 1469 | | G * G * A * T * G * mA * mA * mC * mU * mG | | |
| WV- | GGCAUCCTCGGCCTCUGAAG | mG * mG * mC * mA * mU * C * C * T * C * G * | XXXXXXXXXXXXXXXXXXX | 1271 |
| 1470 | | G * C * C * T * C * mU * mG * mA * mA * mG | | |
| WV- | UUGGUGGCGTGCTTCAUGUA | mU * mU * mG * mG * mU * G * G * C * G * T | XXXXXXXXXXXXXXXXXXX | 1272 |
| 1471 | | * G * C * T * T * C * mA * mU * mG * mU * mA | | |
| WV- | GCGUGCTTCATGTAACCCUG | mG * mC * mG * mU * mG * C * T * T * C * A * | XXXXXXXXXXXXXXXXXXX | 1273 |
| 1472 | | T * G * T * A * A * mC * mC * mC * mU * mG | | |
| WV- | UGAGAAGGGAGGCATCCUCG | mU * mG * mA * mG * mA * A * G * G * G * A | XXXXXXXXXXXXXXXXXXX | 1274 |
| 1473 | | * G * G * C * A * T * mC * mC * mU * mC * mG | | |
| WV- | GCUGAAGTTGGTCTGACCUC | mG * mC * mU * mG * mA * A * G * T * T * G * | XXXXXXXXXXXXXXXXXXX | 1275 |
| 1474 | | G * T * C * T * G * mA * mC * mC * mU * mC | | |
| WV- | GGGCCTCCCAAGGCAAACCC | mG * mG * mG * mC * mC * T * C * C * C * A * | XXXXXXXXXXXXXXXXXXX | 1276 |
| 1475 | | A * G * G * C * A * mA * mA * mC * mC * mC | | |
| WV- | GUUUATGCCCCTGGGCCUGA | mG * mU * mU * mU * mA * T * G * C * C * C * | XXXXXXXXXXXXXXXXXXX | 1277 |
| 1476 | | C * T * G * G * G * mC * mC * mU * mG * mA | | |
| WV- | AACCUTAGCTGGGTCUGCCA | mA * mA * mC * mC * mU * T * A * G * C * T * | XXXXXXXXXXXXXXXXXXX | 1278 |
| 1477 | | G * G * G * T * C * mU * mG * mC * mC * mA | | |
| WV- | CACCCATTGGGACTGGGAUC | mC * mA * mC * mC * mC * A * T * T * G * G * | XXXXXXXXXXXXXXXXXXX | 1279 |
| 1478 | | G * A * C * T * G * mG * mG * mA * mU * mC | | |
| WV- | CUCCUGCTTGACCACCCAUU | mC * mU * mC * mC * mU * G * C * T * T * G * | XXXXXXXXXXXXXXXXXXXX | 1280 |
| 1479 | | A * C * C * A * C * mC * mC * mA * mU * mU | | |
| WV- | GCUCCTGCTTGACCACCCAU | mG * mC * mU * mC * mC * T * G * C * T * T * | XXXXXXXXXXXXXXXXXXXX | 1281 |
| 1480 | | G * A * C * C * A * mC * mC * mC * mA * mU | | |
| WV- | UGGGCTCCTGCTTGACCACC | mU * mG * mG * mG * mC * T * C * C * T * G * | XXXXXXXXXXXXXXXXXXXX | 1282 |
| 1481 | | C * T * T * G * A * mC * mC * mA * mC * mC | | |
| WV- | TGUCCAGCUUUAUUGG | T * SfG * SmUfC * SmCfA * SmGfC * SmU * SfU | SSO SO SO SSSO | 1283 |
| 8070 | GAGTUTTTTTGG | * SmUfA * SmU * SfU * SmG * SfG * SmG * SfA | SSSSSSSSSOOOOO | |
| | TAATCCACTTTCAGAGG | * SmG * ST * SmUTTTTTeo * Geo * Geo * Teo | XXXXXXXXXXXXXXXXXXXO | |
| | | * Aeo * A * T * m5C * m5C * A * m5C * T * T * | | |
| | | T * m5C * Aeo * Geo * Aeo * Geo * | | |
| | | GeoL003Mod001 | | |
| WV- | TGUCCAGCUUUAUUGG | T * SfG * SmUfC * SmCfA * SmGfC * SmU * SfU | SSO SO SO SSSO | 1284 |
| 8068 | GAGTUTTTTTGG | * SmUfA * SmU * SfU * SmG * SfG * SmG * SfA | SSSSSSSSSOOOOO | |
| | TAATCCACTTTCAGAGG | * SmG * STGaNC6T * SmUTTTTTeo * Geo * | XXXXXXXXXXXXXXXXXXX | |
| | | Geo * Teo * Aeo * A * T * m5C * m5C * A * | | |
| | | m5C * T * T * T * m5C * Aeo * Geo * Aeo * | | |
| | | Geo * Geo | | |
| WV- | TGUCCAGCUUUAUUGG | VPT * SfG * SmUfC * SmCfA * SmGfC * SmU * | SSO SO SO SSSO | 1285 |
| 8066 | GAGTUTTTTTGG | SfU * SmUfA * SmU * SfU * SmG * SfG * SmG | SSSSSSSSSOOOOO | |
| | TAATCCACTTTCAGAGG | * SfA * SmG * ST * SmUTTTTTeo * Geo * Geo * | XXXXXXXXXXXXXXXXXXXO | |
| | | Teo * Aeo * A * T * m5C * m5C * A * m5C * T | | |
| | | * T * T * m5C * Aeo * Geo * Aeo * Geo * | | |
| | | GeoL003Mod001 | | |
| WV- | TGUCCAGCUUUAUUGG | VPT * SfG * SmUfC * SmCfA * SmGfC * SmU * | SSO SO SO SSSO | 1286 |
| 8064 | GAGTUTTTTTGG | SfU * SmUfA * SmU * SfU * SmG * SfG * SmG | SSSSSSSSSOOOOO | |
| | TAATCCACTTTCAGAGG | * SfA * SmG * STGaNC6T * SmUTTTTTeo * Geo | XXXXXXXXXXXXXXXXXXX | |
| | | * Geo * Teo * Aeo * A * T * m5C * m5C * A * | | |
| | | m5C * T * T * T * m5C * Aeo * Geo * Aeo * | | |
| | | Geo * Geo | | |
| WV- | TGUCCAGCUUUAUUGG | VPT * fG * mUfC * mCfA * mGfC * mUfU * | XXO XO XO XO XO XO | 1287 |
| 8062 | GAGTUTTTTTGG | mUfA * mUfU * mG * fG * mG * fA * mG * | XXXXXXXOOOOO | |
| | TAATCCACTTTCAGAGG | TGaNC6T * mUTTTTTeo * Geo * Geo * Teo * | XXXXXXXXXXXXXXXXXXX | |
| | | Aeo * A * T * m5C * m5C * A * m5C * T * T * T | | |
| | | * m5C * Aeo * Geo * Aeo * Geo * Geo | | |
| WV- | TGUCCAGCUUUAUUGGGAGT | VPT * SfG * SmUfC * SmCfA * SmGfC * SmU * | SSOSOSOSSSOSSSSSSSSSO | 1288 |
| 8064 | UTT | SfU * SmUfA * SmU * SfU * SmG * SfG * SmG | O | |
| | TTTGGTAATCCACTTTCAGAGG | * SfA * SmG * STGaNC6T * SmUTTTTTeo * Geo | OOOXXXXXXXXXXXXXXXXX | |
| | | * Geo * Teo * Aeo * A * T * m5C * m5C * A * | XX | |
| | | m5C * T * T * T * m5C * Aeo * Geo * Aeo * | | |
| | | Geo * Geo | | |
| WV- | TGUCCAGCUUUAUUGGGAGT | VPT * SfG * SmUfC * SmCfA * SmGfC * SmU * | SSOSOSOSSSOSSSSSSSSSO | 1289 |
| 8066 | UTT | SfU * SmUfA * SmU * SfU * SmG * SfG * SmG | O | |
| | TTTGGTAATCCACTTTCAGAGG | * SfA * SmG * ST * SmUTTTTTeo * Geo * Geo * | OOOXXXXXXXXXXXXXXXXX | |
| | | Teo * Aeo * A * T * m5C * m5C * A * m5C * T | XXO | |
| | | * T * T * m5C * Aeo * Geo * Aeo * Geo * | | |
| | | GeoL003Mod001 | | |
| WV- | TGUCCAGCUUUAUUGGGAGT | T * SfG * SmUfC * SmCfA * SmGfC * SmU * SfU | SSOSOSOSSSOSSSSSSSSSO | 1290 |
| 8068 | UTT | * SmUfA * SmU * SfU * SmG * SfG * SmG * SfA | O | |
| | TTTGGTAATCCACTTTCAGAGG | * SmG * STGaNC6T * SmUTTTTTeo * Geo * | OOOXXXXXXXXXXXXXXXXX | |
| | | Geo * Teo * Aeo * A * T * m5C * m5C * A * | XX | |
| | | m5C * T * T * T * m5C * Aeo * Geo * Aeo * | | |
| | | Geo * Geo | | |
| WV- | TGUCCAGCUUUAUUGGGAGT | T * SfG * SmUfC * SmCfA * SmGfC * SmU * SfU | SSOSOSOSSSOSSSSSSSSSO | 1291 |
| 8070 | UTTT | * SmUfA * SmU * SfU * SmG * SfG * SmG * SfA | O | |
| | TTGGTAATCCACTTTCAGAGG | * SmG * ST * SmUTTTTTeo * Geo * Geo * Teo | OOOXXXXXXXXXXXXXXXXX | |
| | | * Aeo * A * T * m5C * m5C * A * m5C * T * T * | XXO | |
| | | T * m5C * Aeo * Geo * Aeo * Geo * | | |
| | | GeoL003Mod001 | | |
| WV- | TGUCCAGCUUUAUUGGGAGG | P05MRdT * SfG * SmUfC * SmCfA * SmGfC * | SSOSOSOSSSOSSSSSSSSSS | 1292 |
| 8242 | CTU | SmU * SfU * SmUfA * SmU * SfU * SmG * SfG | S | |
| | | * SmG * SfA * SmG * SfG * SmC * STGaNC6T * | | |
| | | SmU | | |
| WV- | TAGCUUCUUGUCCAGCUUUA | PO5MRdT * SfA * SmGfC * SmU * SfU * SmC * | SSOSSSSSSSOSOSSSSSSSSS | 1293 |
| 8243 | UTU | SfU * SmU * SfG * SmUfC * SmCfA * SmG * SfC | | |
| | | * SmU * SfU * SmU * SfA * SmU * STGaNC6T * | | |
| | | SmU | | |
| WV- | TGUCCAGCUUUAUUGGGAGT | VPT * fG * mUfC * mCfA * mGfC * mUfU * | XXOXOXOXOXOXOXXXXXX | 1294 |
| 8254 | UT | mUfA * mUfU * mG * fG * mG * fA * mG * | X | |
| | TTTTGGTAATCCACTTTCAGAG | AMC6T * mUTTTTTeo * Geo * Geo * Teo * Aeo | OOOOXXXXXXXXXXXXXX | |
| | G | * A * T * m5C * m5C * A * m5C * T * T * T * | XXXXX | |
| | | m5C * Aeo * Geo * Aeo * Geo * Geo | | |
| WV- | TGUCCAGCUUUAUUGGGAGG | PO5MRdT * SfG * SmUfC * SmCfA * SmGfC * | SSOSOSOSSSOSSSSSSSSSSS | 1295 |
| 8261 | CTU | SmU * SfU * SmUfA * SmU * SfU * SmG * SfG | | |
| | | * SmG * SfA * SmG * SfG * SmC * SAMC6T * | | |
| | | SmU | | |
| WV- | TAGCUUCUUGUCCAGCUUUA | PO5MRdT * SfA * SmGfC * SmU * SfU * SmC * | SSOSSSSSSSOSOSSSSSSSSS | 1296 |
| 8262 | UTU | SfU * SmU * SfG * SmUfC * SmCfA * SmG * SfC | | |
| | | * SmU * SfU * SmU * SfA * SmU * SAMC6T * | | |
| | | SmU | | |
| WV- | TGUCCAGCUUUAUUGGGAGG | PS5MRdT * SfG * SmUfC * SmCfA * SmGfC * | SSOSOSOSSSOSSSSSSSSSSS | 1297 |
| 8281 | CTU | SmU * SfU * SmUfA * SmU * SfU * SmG * SfG | | |
| | | * SmG * SfA * SmG * SfG * SmC * STGaNC6T * | | |
| | | SmU | | |
| WV- | TGUCCAGCUUUAUUGGGAGG | PH5MRdT * SfG * SmUfC * SmCfA * SmGfC * | SSOSOSOSSSOSSSSSSSSSSS | 1298 |
| 8282 | CTU | SmU * SfU * SmUfA * SmU * SfU * SmG * SfG | | |
| | | * SmG * SfA * SmG * SfG * SmC * STGaNC6T * | | |
| | | SmU | | |
| WV- | TGUCCAGCUUUAUUGGGAGG | 5MRdT * SfG * SmUfC * SmCfA * SmGfC * | SSOSOSOSSSOSSSSSSSSSSS | 1299 |
| 8283 | CTU | SmU * SfU * SmUfA * SmU * SfU * SmG * SfG | | |
| | | * SmG * SfA * SmG * SfG * SmC * STGaNC6T * | | |
| | | SmU | | |
| WV- | TGUCCAGCUUUAUUGGGAGG | PS5MRdT * SfG * SmUfC * SmCfA * SmGfC * | SSOSOSOSSSOSSSSSSSSSSS | 1300 |
| 8284 | CTU | SmU * SfU * SmUfA * SmU * SfU * SmG * SfG | | |
| | | * SmG * SfA * SmG * SfG * SmC * SAMC6T * | | |
| | | SmU | | |
| WV- | TGUCCAGCUUUAUUGGGAGG | PH5MRdT * SfG * SmUfC * SmCfA * SmGfC * | SSOSOSOSSSOSSSSSSSSSSS | 1301 |
| 8285 | CTU | SmU * SfU * SmUfA * SmU * SfU * SmG * SfG | | |
| | | * SmG * SfA * SmG * SfG * SmC * SAMC6T * | | |
| | | SmU | | |
| WV- | TGUCCAGCUUUAUUGGGAGG | 5MRdT * SfG * SmUfC * SmCfA * SmGfC * | SSOSOSOSSSOSSSSSSSSSSS | 1302 |
| 8286 | CTU | SmU * SfU * SmUfA * SmU * SfU * SmG * SfG | | |
| | | * SmG * SfA * SmG * SfG * SmC * SAMC6T * | | |
| | | SmU | | |
| WV- | TGUCCAGCUUUAUUGGGAGG | Mod001L001T * fG * mUfC * mCfA * mGfC * | OXXOXOXOXOXOXOXXXXX | 1303 |
| 8325 | CTU | mUfU * mUfA * mUfU * mG * fG * mG * fA * | XXXX | |
| | | mG * fG * mC * T * mU | | |
| WV- | TGUCCAGCUUUAUUGGGAGG | Mod001L001 * T * fG * mUfC * mCfA * mGfC * | XXXOXOXOXOXOXOXXXXX | 1304 |
| 8326 | CTU | mUfU * mUfA * mUfU * mG * fG * mG * fA * | XXXX | |
| | | mG * fG * mC * T * mU | | |
| WV- | TAGCUUCUUGUCCAGCUUUA | Mod001L001T * fA * mGfC * mUfU * mCfU * | OXXOXOXOXOXOXOXXXXX | 1305 |
| 8327 | UTU | mUfG * mUfC * mCfA * mG * fC * mU * fU * | XXXX | |
| | | mU * fA * mU * T * mU | | |
| WV- | TAGCUUCUUGUCCAGCUUUA | Mod001L001 * T * fA * mGfC * mUfU * mCfU | XXXOXOXOXOXOXOXXXXX | 1306 |
| 8328 | UTU | * mUfG * mUfC * mCfA * mG * fC * mU * fU * | XXXX | |
| | | mU * fA * mU * T * mU | | |
| WV- | TGUCCAGCUUUAUUGGGAGG | L001T * fG * mUfC * mCfA * mGfC * mUfU * | OXXOXOXOXOXOXOXXXXX | 1307 |
| 8330 | CTU | mUfA * mUfU * mG * fG * mG * fA * mG * fG | XXXX | |
| | | * mC * T * mU | | |
| WV- | TGUCCAGCUUUAUUGGGAGG | L001 * T * fG * mUfC * mCfA * mGfC * mUfU * | XXXOXOXOXOXOXOXXXXX | 1308 |
| 8331 | CTU | mUfA * mUfU * mG * fG * mG * fA * mG * fG | XXXX | |
| | | * mC * T * mU | | |
| WV- | TAGCUUCUUGUCCAGCUUUA | L001T * fA * mGfC * mUfU * mCfU * mUfG * | OXXOXOXOXOXOXOXXXXX | 1309 |
| 8332 | UTU | mUfC * mCfA * mG * fC * mU * fU * m U * fA * | XXXX | |
| | | mU * T * mU | | |
| WV- | TAGCUUCUUGUCCAGCUUUA | L001 * T * fA * mGfC * mUfU * mCfU * mUfG * | XXXOXOXOXOXOXOXXXXX | 1310 |
| 8333 | UTU | mUfC * mCfA * mG * fC * mU * fU * m U * fA * | XXXX | |
| | | mU * T * mU | | |
| WV- | TGUCCAGCUUUAUUGGGAGG | T * fG * mUmC * mCmA * mGmC * mU * mU * | XXOXOXOXXXOXXXXXXXXX | 1311 |
| 8427 | CTU | mUmA * mU * fU * mG * mG * mG * mA * mG | XX | |
| | | * mG * mC * T * mU | | |
| WV- | TAGCUUCUUGUCCAGCUUUA | T * fA * mGmC * mU * mU * mC * mU * mU * | XXOXXXXXXXOXOXXXXXXX | 1312 |
| 8428 | UTU | mG * mUmC * mCfA * mG * mC * mU * mU * | XX | |
| | | mU * mA * mU * T * mU | | |
| WV- | TGUCCAGCUUUAUUGGGAGG | T * SfG * SmUmC * SmCmA * SmGmC * | SSOSOSOSOSOSOSSSSSSSS | 1313 |
| 8429 | CTU | SmUmU * SmUmA * SmUfU * SmG * SmG * | S | |
| | | SmG * SmA * SmG * SmG * SmC * ST * SmU | | |
| WV- | TAGCUUCUUGUCCAGCUUUA | T * SfA * SmGmC * SmUmU * SmCmU * | SSOSOSOSOSOSOSSSSSSSS | 1314 |
| 8430 | UTU | SmUmG * SmUmC * SmCfA * SmG * SmC * | S | |
| | | SmU * SmU * SmU * SmA * SmU * ST * SmU | | |
| WV- | TGUCCAGCUUUAUUGGGAGG | T * SfG * SmUmC * SmCmA * SmGmC * SmU * | SSOSOSOSSSOSSSSSSSSSSS | 1315 |
| 8431 | CTU | SmU * SmUmA * SmU * SfU * SmG * SmG * | | |
| | | SmG * SmA * SmG * SmG * SmC * ST * SmU | | |
| WV- | TAGCUUCUUGUCCAGCUUUA | T * SfA * SmGmC * SmU * SmU * SmC * SmU * | SSOSSSSSSSOSOSSSSSSSSS | 1316 |
| 8432 | UTU | SmU * SmG * SmUmC * SmCfA * SmG * SmC * | | |
| | | SmU * SmU * SmU * SmA * SmU * ST * SmU | | |
| WV- | AGCTUCTTGTCCAGCUUUAU | Mod001L001mA * SGeom5CeoTeomU * SC * | OSOOOSSSSRSSRSSSSSSS | 1317 |
| 8610 | | ST * ST * SG * RT * SC * SC * RA * SG * SC * | | |
| | | SmU * SmU * SmU * SmA * SmU | | |
| WV- | AGCTUCTTGTCCAGCUUUAU | Mod001L001mA * SGeom5CeoTeomU * SC * | OSOOOSSSSRSSSSSSSSSS | 1318 |
| 8611 | | ST * ST * SG * RT * SC * SC * SA * SG * SC * | | |
| | | SmU * SmU * SmU * SmA * SmU | | |
| WV- | AGCTUCTTGTCCAGCUUUAU | Mod001L001mA * SGeom5CeoTeomU * SC * | OSOOOSSSSSSSSRSSSSSS | 1319 |
| 8612 | | ST * ST * SG * ST * SC * SC * SA * RG * SC * | | |
| | | SmU * SmU * SmU * SmA * SmU | | |
| WV- | AGCTUCTTGTCCAGCUUUAU | Mod001L001mA * Geom5CeoTeomU * C * T * | OXOOOXXXXXXXXXXXXXXX | 1320 |
| 8613 | | T * G * T * C * C * A * G * C * mU * mU * mU * | | |
| | | mA * mU | | |
| WV- | AGCTUCTTGTCCAGCUUUAU | mA * SGeom5CeoTeomU * SC * ST * ST * SG * | SOOOSSSSRSSRSSSSSSS | 1321 |
| 8614 | | RT * SC * SC * RA * SG * SC * SmU * SmU * | | |
| | | SmU * SmA * SmU | | |
| WV- | AGCTUCTTGTCCAGCUUUAU | mA * SGeom5CeoTeomU * SC * ST * ST * SG * | SOOOSSSSRSSSSSSSSSS | 1322 |
| 8615 | | RT * SC * SC * SA * SG * SC * SmU * SmU * | | |
| | | SmU * SmA * SmU | | |
| WV- | AGCTUCTTGTCCAGCUUUAU | mA * SGeom5CeoTeomU * SC * ST * ST * SG * | SOOOSSSSSSSSRSSSSSS | 1323 |
| 8616 | | ST * SC * SC * SA * RG * SC * SmU * SmU * | | |
| | | SmU * SmA * SmU | | |
| WV- | AGCTUCTTGTCCAGCUUUAU | mA * Geom5CeoTeomU * C * T * T * G * T * C | XOOOXXXXXXXXXXXXXXX | 1324 |
| 8617 | | * C * A * G * C * mU * mU * mU * mA * mU | | |
| WV- | AGCTUCTTGTCCAGCUUUAU | Mod001L001mA * SGeom5CeoTeomU * SC * | OSOOOSSSSSSSRSSSSSSS | 1325 |
| 8618 | | ST * ST * SG * ST * SC * SC * RA * SG * SC * | | |
| | | SmU * SmU * SmU * SmA * SmU | | |
| WV- | AGCTUCTTGTCCAGCUUUAU | mA * SGeom5CeoTeomU * SC * ST * ST * SG * | SOOOSSSSSSSRSSSSSSS | 1326 |
| 8619 | | ST * SC * SC * RA * SG * SC * SmU * SmU * | | |
| | | SmU * SmA * SmU | | |
| WV- | AGCTUCTTGTCCAGCUUUAU | L001mA * SGeom5CeoTeomU * SC * ST * ST * | OSOOOSSSSRSSRSSSSSSS | 1327 |
| 8629 | | SG * RT * SC * SC * RA * SG * SC * SmU * SmU | | |
| | | * SmU * SmA * SmU | | |
| WV- | AGCTUCTTGTCCAGCUUUAU | L001mA * SGeom5CeoTeomU * SC * ST * ST * | OSOOOSSSSRSSSSSSSSSS | 1328 |
| 8630 | | SG * RT * SC * SC * SA * SG * SC * SmU * SmU | | |
| | | * SmU * SmA * SmU | | |
| WV- | AGCTUCTTGTCCAGCUUUAU | L001mA * SGeom5CeoTeomU * SC * ST * ST * | OSOOOSSSSSSSRSSSSSSS | 1329 |
| 8631 | | SG * ST * SC * SC * RA * SG * SC * SmU * SmU | | |
| | | * SmU * SmA * SmU | | |
| WV- | AGCTUCTTGTCCAGCUUUAU | L001mA * Geom5CeoTeomU * C * T * T * G * | OXOOOXXXXXXXXXXXXXXX | 1330 |
| 8632 | | T * C * C * A * G * C * mU * mU * mU * mA * | | |
| | | mU | | |
| WV- | CTTGTCCAGCTTTATTGGGA | Mod001L001m5Ceo * STeoTeoGeoTeo * SC * | OSOOOSSSSSRSSRSSOOOS | 1331 |
| 8633 | | SC * SA * SG * SC * RT * ST * ST * RA * ST * | | |
| | | STeoGeoGeoGeo * SAeo | | |
| WV- | CTTGTCCAGCTTTATTGGGA | Mod001L001m5Ceo * STeoTeoGeoTeo * SC * | OSOOOSSSSSRSSSSSOOOS | 1332 |
| 8634 | | SC * SA * SG * SC * RT * ST * ST * SA * ST * | | |
| | | STeoGeoGeoGeo * SAeo | | |
| WV- | CTTGTCCAGCTTTATTGGGA | Mod001L001m5Ceo * STeoTeoGeoTeo * SC * | OSOOOSSSSSSSSRSSOOOS | 1333 |
| 8635 | | SC * SA * SG * SC * ST * ST * ST * RA * ST * | | |
| | | STeoGeoGeoGeo * SAeo | | |
| WV- | CTTGTCCAGCTTTATTGGGA | Mod001L001m5Ceo * TeoTeoGeoTeo * C * C | OXOOOXXXXXXXXXXXOOO | 1334 |
| 8636 | | * A * G * C * T * T * T * A * T * | X | |
| | | TeoGeoGeoGeo * Aeo | | |
| WV- | CTTGTCCAGCTTTATUGGGA | Mod001L001m5Ceo * STeoTeoGeoTeo * SC * | OSOOOSSSSSRSSRSSSSSS | 1335 |
| 8637 | | SC * SA * SG * SC * RT * ST * ST * RA * ST * | | |
| | | SmU * SmG * SmG * SmG * SmA | | |
| WV- | CTTGTCCAGCTTTATUGGGA | Mod001L001m5Ceo * STeoTeoGeoTeo * SC * | OSOOOSSSSSRSSSSSSSSS | 1336 |
| 8638 | | SC * SA * SG * SC * RT * ST * ST * SA * ST * | | |
| | | SmU * SmG * SmG * SmG * SmA | | |
| WV- | CTTGTCCAGCTTTATUGGGA | Mod001L001m5Ceo * STeoTeoGeoTeo * SC * | OSOOOSSSSSSSSRSSSSSS | 1337 |
| 8639 | | SC * SA * SG * SC * ST * ST * ST * RA * ST * | | |
| | | SmU * SmG * SmG * SmG * SmA | | |
| WV- | CTTGTCCAGCTTTATUGGGA | Mod001L001m5Ceo * TeoTeoGeoTeo * C * C | OXOOOXXXXXXXXXXXXXXX | 1338 |
| 8640 | | * A * G * C * T * T * T * A * T * mU * mG * mG | | |
| | | * mG * mA | | |
| WV- | ATAGCAGCTTCTTGTCCAGC | Mod001L001Aeo * STeoAeoGeom5Ceo * SA * | OSOOOSSSSSRSSRSSOOOS | 1339 |
| 8641 | | SG * SC * ST * ST * RC * ST * ST * RG * ST * | | |
| | | Sm5Ceom5CeoAeoGeo * 5m5CeO | | |
| WV- | ATAGCAGCTTCTTGTCCAGC | Mod001L001Aeo * STeoAeoGeom5Ceo * SA * | OSOOOSSSSSRSSSSSOOOS | 1340 |
| 8642 | | SG * SC * ST * ST * RC * ST * ST * SG * ST * | | |
| | | Sm5Ceom5CeoAeoGeo * 5m5CeO | | |
| WV- | ATAGCAGCTTCTTGTCCAGC | Mod001L001Aeo * STeoAeoGeom5Ceo * SA * | OSOOOSSSSSSSSRSSOOOS | 1341 |
| 8643 | | SG * SC * ST * ST * SC * ST * ST * RG * ST * | | |
| | | Sm5Ceom5CeoAeoGeo * 5m5CeO | | |
| WV- | ATAGCAGCTTCTTGTCCAGC | Mod001L001Aeo * TeoAeoGeom5Ceo * A * G | OXOOOXXXXXXXXXXXOOO | 1342 |
| 8644 | | * C * T * T * C * T * T * G * T * | X | |
| | | m5Ceom5CeoAeoGeo * m5Ceo | | |
| WV- | ATAGCAGCTTCTTGTCCAGC | Mod001L001Aeo * STeoAeoGeom5Ceo * SA * | OSOOOSSSSSRSSRSSSSSS | 1343 |
| 8645 | | SG * SC * ST * ST * RC * ST * ST * RG * ST * | | |
| | | Sm5C * Sm5C * SmA * SmG * Sm5C | | |
| WV- | ATAGCAGCTTCTTGTCCAGC | Mod001L001Aeo * STeoAeoGeom5Ceo * SA * | OSOOOSSSSSRSSSSSSSSS | 1344 |
| 8646 | | SG * SC * ST * ST * RC * ST * ST * SG * ST * | | |
| | | Sm5C * Sm5C * SmA * SmG * Sm5C | | |
| WV- | ATAGCAGCTTCTTGTCCAGC | Mod001L001Aeo * STeoAeoGeom5Ceo * SA * | OSOOOSSSSSSSSRSSSSSS | 1345 |
| 8647 | | SG * SC * ST * ST * SC * ST * ST * RG * ST * | | |
| | | Sm5C * Sm5C * SmA * SmG * Sm5C | | |
| WV- | ATAGCAGCTTCTTGTCCAGC | Mod001L001Aeo * TeoAeoGeom5Ceo * A * G | OXOOOXXXXXXXXXXXXXXX | 1346 |
| 8648 | | * C * T * T * C * T * T * G * T * m5C * m5C * | | |
| | | mA * mG * m5C | | |
| WV- | CTTGTCCAGCTTTATTGGGA | m5Ceo * STeoTeoGeoTeo * SC * SC * SA * SG | SOOOSSSSSRSSRSSOOOS | 1347 |
| 8649 | | * SC * RT * ST * ST * RA * ST * | | |
| | | STeoGeoGeoGeo * SAeo | | |
| WV- | CTTGTCCAGCTTTATTGGGA | m5Ceo * STeoTeoGeoTeo * SC * SC * SA * SG | SOOOSSSSSRSSSSSOOOS | 1348 |
| 8650 | | * SC * RT * ST * ST * SA * ST * | | |
| | | STeoGeoGeoGeo * SAeo | | |
| WV- | CTTGTCCAGCTTTATTGGGA | m5Ceo * STeoTeoGeoTeo * SC * SC * SA * SG | SOOOSSSSSSSSRSSOOOS | 1349 |
| 8651 | | * SC * ST * ST * ST * RA * ST * | | |
| | | STeoGeoGeoGeo * SAeo | | |
| WV- | CTTGTCCAGCTTTATTGGGA | m5Ceo * TeoTeoGeoTeo * C * C * A * G * C * T | XOOOXXXXXXXXXXXOOOX | 1350 |
| 8652 | | * T * T * A * T * TeoGeoGeoGeo * Aeo | | |
| WV- | CTTGTCCAGCTTTATUGGGA | m5Ceo * STeoTeoGeoTeo * SC * SC * SA * SG | SOOOSSSSSRSSRSSSSSS | 1351 |
| 8653 | | * SC * RT * ST * ST * RA * ST * SmU * SmG * | | |
| | | SmG * SmG * SmA | | |
| WV- | CTTGTCCAGCTTTATUGGGA | m5Ceo * STeoTeoGeoTeo * SC * SC * SA * SG | SOOOSSSSSRSSSSSSSSS | 1352 |
| 8654 | | * SC * RT * ST * ST * SA * ST * SmU * SmG * | | |
| | | SmG * SmG | * SmA | |
| WV- | CTTGTCCAGCTTTATUGGGA | m5Ceo * STeoTeoGeoTeo * SC * SC * SA * SG | SOOOSSSSSSSSRSSSSSS | 1353 |
| 8655 | | * SC * ST * ST * ST * RA * ST * SmU * SmG * | | |
| | | SmG * SmG * SmA | | |
| WV- | CTTGTCCAGCTTTATUGGGA | m5Ceo * TeoTeoGeoTeo * C * C * A * G * C * T | XOOOXXXXXXXXXXXXXXX | 1354 |
| 8656 | | * T * T * A * T * mU * mG * mG * mG * mA | | |
| WV- | ATAGCAGCTTCTTGTCCAGC | Aeo * STeoAeoGeom5Ceo * SA * SG * SC * ST | SOOOSSSSSRSSRSSOOOS | 1355 |
| 8657 | | * ST * RC * ST * ST * RG * ST * | | |
| | | Sm5Ceom5CeoAeoGeo * Sm5Ceo | | |
| WV- | ATAGCAGCTTCTTGTCCAGC | Aeo * STeoAeoGeom5Ceo * SA * SG * SC * ST | SOOOSSSSSRSSSSSOOOS | 1356 |
| 8658 | | * ST * RC * ST * ST * SG * ST * | | |
| | | Sm5Ceom5CeoAeoGeo * 5m5CeO | | |
| WV- | ATAGCAGCTTCTTGTCCAGC | Aeo * STeoAeoGeom5Ceo * SA * SG * SC * ST | SOOOSSSSSSSSRSSOOOS | 1357 |
| 8659 | | * ST * SC * ST * ST * RG * ST * | | |
| | | Sm5Ceom5CeoAeoGeo * 5m5CeO | | |
| WV- | ATAGCAGCTTCTTGTCCAGC | Aeo * TeoAeoGeom5Ceo * A * G * C * T * T * | XOOOXXXXXXXXXXXOOOX | 1358 |
| 8660 | | C * T * T * G * T * m5Ceom5CeoAeoGeo * | | |
| | | m5Ceo | | |
| WV- | ATAGCAGCTTCTTGTCCAGC | Aeo * STeoAeoGeom5Ceo * SA * SG * SC * ST | SOOOSSSSSRSSRSSSSSS | 1359 |
| 8661 | | * ST * RC * ST * ST * RG * ST * Sm5C * Sm5C * | | |
| | | SmA * SmG * Sm5C | | |
| WV- | ATAGCAGCTTCTTGTCCAGC | Aeo * STeoAeoGeom5Ceo * SA * SG * SC * ST | SOOOSSSSSRSSSSSSSSS | 1360 |
| 8662 | | * ST * RC * ST * ST * SG * ST * Sm5C * Sm5C * | | |
| | | SmA * SmG * Sm5C | | |
| WV- | ATAGCAGCTTCTTGTCCAGC | Aeo * STeoAeoGeom5Ceo * SA * SG * SC * ST | SOOOSSSSSSSSRSSSSSS | 1361 |
| 8663 | | * ST * SC * ST * ST * RG * ST * Sm5C * Sm5C * | | |
| | | SmA * SmG * Sm5C | | |
| WV- | ATAGCAGCTTCTTGTCCAGC | Aeo * TeoAeoGeom5Ceo * A * G * C * T * T * | XOOOXXXXXXXXXXXXXXX | 1362 |
| 8664 | | C * T * T * G * T * m5C * m5C * mA * mG * | | |
| | | m5C | | |
| WV- | ATAGCAGCTTCTTGTCCAGC | Mod001L001Aeo * STeoAeoGeom5Ceo * SA * | OSOOOSSSSSRSSRSSSSSS | 1363 |
| 8665 | | SG * SC * ST * ST * RC * ST * ST * RG * ST * | | |
| | | SmC * SmC * SmA * SmG * SmC | | |
| WV- | ATAGCAGCTTCTTGTCCAGC | Mod001L001Aeo * STeoAeoGeom5Ceo * SA * | OSOOOSSSSSRSSSSSSSSS | 1364 |
| 8666 | | SG * SC * ST * ST * RC * ST * ST * SG * ST * | | |
| | | SmC * SmC * SmA * SmG * SmC | | |
| WV- | ATAGCAGCTTCTTGTCCAGC | Mod001L001Aeo * STeoAeoGeom5Ceo * SA * | OSOOOSSSSSSSSRSSSSSS | 1365 |
| 8667 | | SG * SC * ST * ST * SC * ST * ST * RG * ST * | | |
| | | SmC * SmC * SmA * SmG * SmC | | |
| WV- | ATAGCAGCTTCTTGTCCAGC | Mod001L001Aeo * TeoAeoGeom5Ceo * A * G | OXOOOXXXXXXXXXXXXXXX | 1366 |
| 8668 | | * C * T * T * C * T * T * G * T * mC * mC * mA | | |
| | | mG * mC | | |
| WV- | ATAGCAGCTTCTTGTCCAGC | Aeo * STeoAeoGeom5Ceo * SA * SG * SC * ST | SOOOSSSSSRSSRSSSSSS | 1367 |
| 8669 | | * ST * RC * ST * ST * RG * ST * SmC * SmC * | | |
| | | SmA * SmG * SmC | | |
| WV- | ATAGCAGCTTCTTGTCCAGC | Aeo * STeoAeoGeom5Ceo * SA * SG * SC * ST | SOOOSSSSSRSSSSSSSSS | 1368 |
| 8670 | | * ST * RC * ST * ST * SG * ST * SmC * SmC * | | |
| | | SmA * SmG * SmC | | |
| WV- | ATAGCAGCTTCTTGTCCAGC | Aeo * STeoAeoGeom5Ceo * SA * SG * SC * ST | SOOOSSSSSSSSRSSSSSS | 1369 |
| 8671 | | * ST * SC * ST * ST * RG * ST * SmC * SmC * | | |
| | | SmA * SmG * SmC | | |
| WV- | ATAGCAGCTTCTTGTCCAGC | Aeo * TeoAeoGeom5Ceo * A * G * C * T * T * | XOOOXXXXXXXXXXXXXXX | 1370 |
| 8672 | | C * T * T * G * T * mC * mC * mA * mG * mC | | |
| WV- | CTTGUCCAGCTTTATUGGGA | Mod001L001mC * STeoTeoGeomU * SC * SC * | OSOOOSSSSSRSSRSSSSSS | 1371 |
| 8673 | | SA * SG * SC * RT * ST * ST * RA * ST * SmU * | | |
| | | SmG * SmG * SmG * SmA | | |
| WV- | CTTGUCCAGCTTTATUGGGA | Mod001L001mC * STeoTeoGeomU * SC * SC * | OSOOOSSSSSRSSSSSSSSS | 1372 |
| 8674 | | SA * SG * SC * RT * ST * ST * SA * ST * SmU * | | |
| | | SmG * SmG * SmG * SmA | | |
| WV- | CTTGUCCAGCTTTATUGGGA | Mod001L001mC * STeoTeoGeomU * SC * SC * | OSOOOSSSSSSSSRSSSSSS | 1373 |
| 8675 | | SA * SG * SC * ST * ST * ST * RA * ST * SmU * | | |
| | | SmG * SmG * SmG * SmA | | |
| WV- | CTTGUCCAGCTTTATUGGGA | Mod001L001mC * TeoTeoGeomU * C * C * A * | OXOOOXXXXXXXXXXXXXXX | 1374 |
| 8676 | | G * C * T * T * T * A * T * mU * mG * mG * mG | | |
| | | * mA | | |
| WV- | ATAGCAGCTTCTTGTCCAGC | Mod001L001mA * STeoAeoGeomC * SA * SG * | OSOOOSSSSSRSSRSSSSSS | 1375 |
| 8677 | | SC * ST * ST * RC * ST * ST * RG * ST * SmC * | | |
| | | SmC * SmA * SmG * SmC | | |
| WV- | ATAGCAGCTTCTTGTCCAGC | Mod001L001mA * STeoAeoGeomC * SA * SG * | OSOOOSSSSSRSSSSSSSSS | 1376 |
| 8678 | | SC * ST * ST * RC * ST * ST * SG * ST * SmC * | | |
| | | SmC * SmA * SmG * SmC | | |
| WV- | ATAGCAGCTTCTTGTCCAGC | Mod001L001mA * STeoAeoGeomC * SA * SG * | OSOOOSSSSSSSSRSSSSSS | 1377 |
| 8679 | | SC * ST * ST * SC * ST * ST * RG * ST * SmC * | | |
| | | SmC * SmA * SmG * SmC | | |
| WV- | ATAGCAGCTTCTTGTCCAGC | Mod001L001mA * TeoAeoGeomC * A * G * C | OXOOOXXXXXXXXXXXXXXX | 1378 |
| 8680 | | * T * T * C * T * T * G * T * mC * mC * mA * | | |
| | | mG * mC | | |
| WV- | CTTGUCCAGCTTTATUGGGA | mC * STeoTeoGeomU * SC * SC * SA * SG * SC | SOOOSSSSSRSSRSSSSSS | 1379 |
| 8681 | | * RT * ST * ST * RA * ST * SmU * SmG * SmG * | | |
| | | SmG * SmA | | |
| WV- | CTTGUCCAGCTTTATUGGGA | mC * STeoTeoGeomU * SC * SC * SA * SG * SC | SOOOSSSSSRSSSSSSSSS | 1380 |
| 8682 | | * RT * ST * ST * SA * ST * SmU * SmG * SmG * | | |
| | | SmG * SmA | | |
| WV- | CTTGUCCAGCTTTATUGGGA | mC * STeoTeoGeomU * SC * SC * SA * SG * SC | SOOOSSSSSSSSRSSSSSS | 1381 |
| 8683 | | * ST * ST * ST * RA * ST * SmU * SmG * SmG * | | |
| | | SmG * SmA | | |
| WV- | CTTGUCCAGCTTTATUGGGA | mC * TeoTeoGeomU * C * C * A * G * C * T * T | XOOOXXXXXXXXXXXXXXX | 1382 |
| 8684 | | * T * A * T * mU * mG * mG * mG * mA | | |
| WV- | ATAGCAGCTTCTTGTCCAGC | mA * STeoAeoGeomC * SA * SG * SC * ST * ST | SOOOSSSSSRSSRSSSSSS | 1383 |
| 8685 | | * RC * ST * ST * RG * ST * SmC * SmC * SmA * | | |
| | | SmG * SmC | | |
| WV- | ATAGCAGCTTCTTGTCCAGC | mA * STeoAeoGeomC * SA * SG * SC * ST * ST | SOOOSSSSSRSSSSSSSSS | 1384 |
| 8686 | | * RC * ST * ST * SG * ST * SmC * SmC * SmA * | | |
| | | SmG * SmC | | |
| WV- | ATAGCAGCTTCTTGTCCAGC | mA * STeoAeoGeomC * SA * SG * SC * ST * ST | SOOOSSSSSSSSRSSSSSS | 1385 |
| 8687 | | * SC * ST * ST * RG * ST * SmC * SmC * SmA * | | |
| | | SmG * SmC | | |
| WV- | ATAGCAGCTTCTTGTCCAGC | mA * TeoAeoGeomC * A * G * C * T * T * C * T | XOOOXXXXXXXXXXXXXXX | 1386 |
| 8688 | | * T * G * T * mC * mC * mA * mG * mC | | |
| WV- | CTTGTCCAGCTTTATTGGGA | L001m5Ceo * STeoTeoGeoTeo * SC * SC * SA * | OSOOOSSSSSRSSRSSOOOS | 1387 |
| 8822 | | SG * SC * RT * ST * ST * RA * ST * | | |
| | | STeoGeoGeoGeo * SAeo | | |
| WV- | CTTGTCCAGCTTTATTGGGA | L001m5Ceo * STeoTeoGeoTeo * SC * SC * SA * | OSOOOSSSSSRSSSSSOOOS | 1388 |
| 8823 | | SG * SC * RT * ST * ST * SA * ST * | | |
| | | STeoGeoGeoGeo * SAeo | | |
| WV- | CTTGTCCAGCTTTATTGGGA | L001m5Ceo * STeoTeoGeoTeo * SC * SC * SA * | OSOOOSSSSSSSSRSSOOOS | 1389 |
| 8824 | | SG * SC * ST * ST * ST * RA * ST * | | |
| | | STeoGeoGeoGeo * SAeo | | |
| WV- | CTTGTCCAGCTTTATTGGGA | L001m5Ceo * TeoTeoGeoTeo * C * C * A * G * | OXOOOXXXXXXXXXXXOOO | 1390 |
| 8825 | | C * T * T * T * A * T * TeoGeoGeoGeo * Aeo | X | |
| WV- | CTTGUCCAGCTTTATUGGGA | L001mC * STeoTeoGeomU * SC * SC * SA * SG | OSOOOSSSSSRSSRSSSSSS | 1391 |
| 8826 | | * SC * RT * ST * ST * RA * ST * SmU * SmG * | | |
| | | SmG * SmG * SmA | | |
| WV- | CTTGUCCAGCTTTATUGGGA | L001mC * STeoTeoGeomU * SC * SC * SA * SG | OSOOOSSSSSRSSSSSSSSS | 1392 |
| 8827 | | * SC * RT * ST * ST * SA * ST * SmU * SmG * | | |
| | | SmG * SmG * SmA | | |
| WV- | CTTGUCCAGCTTTATUGGGA | L001mC * STeoTeoGeomU * SC * SC * SA * SG | OSOOOSSSSSSSSRSSSSSS | 1393 |
| 8828 | | * SC * ST * ST * ST * RA * ST * SmU * SmG * | | |
| | | SmG * SmG * SmA | | |
| WV- | CTTGUCCAGCTTTATUGGGA | L001mC * TeoTeoGeomU * C * C * A * G * C * | OXOOOXXXXXXXXXXXXXXX | 1394 |
| 8829 | | T * T * T * A * T * mU * mG * mG * mG * mA | | |
| WV- | ATAGCAGCTTCTTGTCCAGC | L001Aeo * STeoAeoGeom5Ceo * SA * SG * SC | OSOOOSSSSSRSSRSSOOOS | 1395 |
| 8830 | | * ST * ST * RC * ST * ST * RG * ST * | | |
| | | Sm5Ceom5CeoAeoGeo * Sm5Ceo | | |
| WV- | ATAGCAGCTTCTTGTCCAGC | L001Aeo * STeoAeoGeom5Ceo * SA * SG * SC | OSOOOSSSSSRSSSSSOOOS | 1396 |
| 8831 | | * ST * ST * RC * ST * ST * SG * ST * | | |
| | | Sm5Ceom5CeoAeoGeo * Sm5Ceo | | |
| WV- | ATAGCAGCTTCTTGTCCAGC | L001Aeo * STeoAeoGeom5Ceo * SA * SG * SC | OSOOOSSSSSSSSRSSOOOS | 1397 |
| 8832 | | * ST * ST * SC * ST * ST * RG * ST * | | |
| | | Sm5Ceom5CeoAeoGeo * Sm5Ceo | | |
| WV- | ATAGCAGCTTCTTGTCCAGC | L001Aeo * TeoAeoGeom5Ceo * A * G * C * T * | OXOOOXXXXXXXXXXXOOO | 1398 |
| 8833 | | T * C * T * T * G * T * m5Ceom5CeoAeoGeo * | X | |
| | | m5Ceo | | |
| WV- | ATAGCAGCTTCTTGTCCAGC | L001mA * STeoAeoGeomC * SA * SG * SC * ST | OSOOOSSSSSRSSRSSSSSS | 1399 |
| 8834 | | * ST * RC * ST * ST * RG * ST * SmC * SmC * | | |
| | | SmA * SmG * SmC | | |
| WV- | ATAGCAGCTTCTTGTCCAGC | L001mA * STeoAeoGeomC * SA * SG * SC * ST | OSOOOSSSSSRSSSSSSSSS | 1400 |
| 8835 | | * ST * RC * ST * ST * SG * ST * SmC * SmC * | | |
| | | SmA * SmG * SmC | | |
| WV- | ATAGCAGCTTCTTGTCCAGC | L001mA * STeoAeoGeomC * SA * SG * SC * ST | OSOOOSSSSSSSSRSSSSSS | 1401 |
| 8836 | | * ST * SC * ST * ST * RG * ST * SmC * SmC * | | |
| | | SmA * SmG * SmC | | |
| WV- | ATAGCAGCTTCTTGTCCAGC | L001mA * TeoAeoGeomC * A * G * C * T * T * | OXOOOXXXXXXXXXXXXXXX | 1402 |
| 8837 | | C * T * T * G * T * mC * mC * mA * mG * mC | | |
| WV- | TGUCCAGCUUUAUUGGGAGG | Mod001L001T * SfG * SmUfC * SmCfA * | OSSOSOSOSSSOSSSSSSSSS | 1403 |
| 8918 | CTU | SmGfC * SmU * SfU * SmUfA * SmU * SfU * | SS | |
| | | SmG * SfG * SmG * SfA * SmG * SfG * SmC * | | |
| | | ST * SmU | | |
| WV- | TGUCCAGCUUUAUUGGGAGG | Mod001L0015MRdT * SfG * SmUfC * SmCfA * | OSSOSOSOSSSOSSSSSSSSS | 1404 |
| 8919 | CTU | SmGfC * SmU * SfU * SmUfA * SmU * SfU * | SS | |
| | | SmG * SfG * SmG * SfA * SmG * SfG * SmC * | | |
| | | ST * SmU | | |
| WV- | TGUCCAGCUUUAUUGGGAGG | POT * SfG * SmUfC * SmCfA * SmGfC * SmU * | SSOSOSOSSSOSSSSSSSSSSS | 1405 |
| 8920 | CTU | SfU * SmUfA * SmU * SfU * SmG * SfG * SmG | | |
| | | * SfA * SmG * SfG * SmC * STGaNC6T * SmU | | |
| WV- | TGUCCAGCUUUAUUGGGAGG | PO5MRdT * SfG * Spac3mUfC * SmCfA * | SSOSOSOSSSOSSSSSSSSSSS | 1406 |
| 8921 | CTU | SmGfC * SmU * SfU * SmUfA * SmU * SfU * | | |
| | | SmG * SfG * SmG * SfA * SmG * SfG * SmC * | | |
| | | STGaNC6T * SmU | | |
| WV- | TGUCCAGCUUUAUUGGGAGG | PO5MRdT * SfG * SmUfC * SmCfA * SmGfC * | SSOSOSOSXSOSSSSSSSSSSS | 1407 |
| 8922 | CTU | Spac3mU * fU * SmUfA * SmU * SfU * SmG | * | |
| | | SfG * SmG * SfA * SmG * SfG * SmC * | | |
| | | STGaNC6T * SmU | | |
| WV- | TGUCCAGCUUUAUUGGGAGG | PO5MRdT * SfG * SmUfC * SmCfA * SmGfC * | SSOSOSOSSSOSSSSSSSSSSS | 1408 |
| 8923 | CTU | SmU * SfU * Spac3mUfA * SmU * SfU * SmG * | | |
| | | SfG * SmG * SfA * SmG * SfG * SmC * | | |
| | | STGaNC6T * SmU | | |
| WV- | TGUCCAGCUUUAUUGGGAGG | PO5MRdT * SfG * SmUfC * SmCfA * SmGfC * | SSOSOSOSSSOSXSSSSSSSSS | 1409 |
| 8924 | CTU | SmU * SfU * SmUfA * Spac3mU * fU * SmG * | | |
| | | SfG * SmG * SfA * SmG * SfG * SmC * | | |
| | | STGaNC6T * SmU | | |
| WV- | TGUCCAGCUUUAUUGGGAGG | PO5MRdT * SfG * SmUfC * SmCfA * SmGfC * | SSOSOSOSSSOSSSSSSSSSSS | 1410 |
| 8925 | UTU | SmU * SfU * SmUfA * SmU * SfU * SmG * SfG | | |
| | | * SmG * SfA * SmG * SfG * Spac3mU * | | |
| | | STGaNC6T * SmU | | |
| WV- | TGUCCAGCUUUAUUGGGAGG | PO5MRdT * SfG * SmUfC * SmCfA * SmGfC * | SSOSOSOSSSOSSSSSSSSSSS | 1411 |
| 8926 | CTU | SmU * SfU * SmUfA * SmU * SfU * SmG * SfG | | |
| | | * SmG * SfA * SmG * SfG * SmC * STGaNC6T * | | |
| | | Spac3mU | | |
| WV- | TGUCCAGCUUUAUUGGGAGG | T * fG * fUmC * mCmA * mGmC * mUmU * | XXOXOXOXOXOXOXXXXXX | 1412 |
| 8938 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * mCmA * mGmC * mUmU * | XXOXOXOXOXOXOXXXXXX | 1413 |
| 8939 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * fCmA * mGmC * mUmU * | XXOXOXOXOXOXOXXXXXX | 1414 |
| 8940 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * mCfA * mGmC * mUmU * | XXOXOXOXOXOXOXXXXXX | 1415 |
| 8941 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * mCmA * fGmC * mUmU * | XXOXOXOXOXOXOXXXXXX | 1416 |
| 8942 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * mCmA * mGfC * mUmU * | XXOXOXOXOXOXOXXXXXX | 1417 |
| 8943 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * mCmA * mGmC * mUmU * | XXOXOXOXOXOXOXXXXXX | 1418 |
| 8944 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * fCmA * mGmC * mUmU * | XXOXOXOXOXOXOXXXXXX | 1419 |
| 8945 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * mCfA * mGmC * mUmU * | XXOXOXOXOXOXOXXXXXX | 1420 |
| 8946 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * mCmA * fGmC * mUmU * | XXOXOXOXOXOXOXXXXXX | 1421 |
| 8947 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * mCmA * mGfC * mUmU * | XXOXOXOXOXOXOXXXXXX | 1422 |
| 8948 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * fCmA * mGmC * mUmU * | XXOXOXOXOXOXOXXXXXX | 1423 |
| 8949 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * mCfA * mGmC * mUmU * | XXOXOXOXOXOXOXXXXXX | 1424 |
| 8950 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * mCmA * fGmC * mUmU * | XXOXOXOXOXOXOXXXXXX | 1425 |
| 8951 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * mCmA * mGfC * mUmU * | XXOXOXOXOXOXOXXXXXX | 1426 |
| 8952 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * fCfA * mGmC * mUmU * | XXOXOXOXOXOXOXXXXXX | 1427 |
| 8953 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * fCmA * fGmC * mUmU * | XXOXOXOXOXOXOXXXXXX | 1428 |
| 8954 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * fCmA * mGfC * mUmU * | XXOXOXOXOXOXOXXXXXX | 1429 |
| 8955 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * mCfA * fGmC * mUmU * | XXOXOXOXOXOXOXXXXXX | 1430 |
| 8956 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * mCfA * mGfC * mUmU * | XXOXOXOXOXOXOXXXXXX | 1431 |
| 8957 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * mCmA * fGfC * mUmU * | XXOXOXOXOXOXOXXXXXX | 1432 |
| 8958 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * fCmA * mGmC * mUmU * | XXOXOXOXOXOXOXXXXXX | 1433 |
| 8959 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * mCfA * mGmC * mUmU * | XXOXOXOXOXOXOXXXXXX | 1434 |
| 8960 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * mCmA * fGmC * mUmU * | XXOXOXOXOXOXOXXXXXX | 1435 |
| 8961 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * mCmA * mGfC * mUmU * | XXOXOXOXOXOXOXXXXXX | 1436 |
| 8962 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * fCfA * mGmC * mUmU * | XXOXOXOXOXOXOXXXXXX | 1437 |
| 8963 | CTU | mUmA * mUfU * mG * mG * mG * mA * mG * | XXX | |
| | | mG * mC * T * mU * | | |
| WV- | TGUCCAGCUUUAUUGGGAGG | T * fG * fUmC * fCmA * fGmC * mUmU * | XXOXOXOXOXOXOXXXXXX | 1438 |
| 8964 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * fCmA * mGfC * mUmU * | XXOXOXOXOXOXOXXXXXX | 1439 |
| 8965 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * mCfA * fGmC * mUmU * | XXOXOXOXOXOXOXXXXXX | 1440 |
| 8966 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * mCfA * mGfC * mUmU * | XXOXOXOXOXOXOXXXXXX | 1441 |
| 8967 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * mCmA * fGfC * mUmU * | XXOXOXOXOXOXOXXXXXX | 1442 |
| 8968 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * fCfA * mGmC * mUmU * | XXOXOXOXOXOXOXXXXXX | 1443 |
| 8969 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * fCmA * fGmC * mUmU * | XXOXOXOXOXOXOXXXXXX | 1444 |
| 8970 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * fCmA * mGfC * mUmU * | XXOXOXOXOXOXOXXXXXX | 1445 |
| 8971 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * mCfA * fGmC * mUmU * | XXOXOXOXOXOXOXXXXXX | 1446 |
| 8972 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * mCfA * mGfC * mUmU * | XXOXOXOXOXOXOXXXXXX | 1447 |
| 8973 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * mCmA * fGfC * mUmU * | XXOXOXOXOXOXOXXXXXX | 1448 |
| 8974 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * fCfA * fGmC * mUmU * | XXOXOXOXOXOXOXXXXXX | 1449 |
| 8975 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * fCfA * mGfC * mUmU * | XXOXOXOXOXOXOXXXXXX | 1450 |
| 8976 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * fCmA * fGfC * mUmU * | XXOXOXOXOXOXOXXXXXX | 1451 |
| 8977 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * mCfA * fGfC * mUmU * | XXOXOXOXOXOXOXXXXXX | 1452 |
| 8978 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * fCfA * mGmC * mUmU * | XXOXOXOXOXOXOXXXXXX | 1453 |
| 8979 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * fCmA * fGmC * mUmU * | XXOXOXOXOXOXOXXXXXX | 1454 |
| 8980 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * fCmA * mGfC * mUmU * | XXOXOXOXOXOXOXXXXXX | 1455 |
| 8981 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * mCfA * fGmC * mUmU * | XXOXOXOXOXOXOXXXXXX | 1456 |
| 8982 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * mCfA * mGfC * mUmU * | XXOXOXOXOXOXOXXXXXX | 1457 |
| 8983 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * mCmA * fGfC * mUmU * | XXOXOXOXOXOXOXXXXXX | 1458 |
| 8984 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * fCfA * fGmC * mUmU * | XXOXOXOXOXOXOXXXXXX | 1459 |
| 8985 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * fCfA * mGfC * mUmU * | XXOXOXOXOXOXOXXXXXX | 1460 |
| 8986 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * fCmA * fGfC * mUmU * | XXOXOXOXOXOXOXXXXXX | 1461 |
| 8987 | CTU | mUmA * mUfU * mG * mG * mG * mA * mG * | XXX | |
| | | mG * mC * T * mU | | |
| WV- | TGUCCAGCUUUAUUGGGAGG | T * fG * fUmC * mCfA * fGfC * mUmU * | XXOXOXOXOXOXOXXXXXX | 1462 |
| 8988 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * fCfA * fGmC * mUmU * | XXOXOXOXOXOXOXXXXXX | 1463 |
| 8989 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * fCfA * mGfC * mUmU * | XXOXOXOXOXOXOXXXXXX | 1464 |
| 8990 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * fCmA * fGfC * mUmU * | XXOXOXOXOXOXOXXXXXX | 1465 |
| 8991 | CTU | mUmA * mUfU * mG * mG * mG * mA * mG * | XXX | |
| | | mG * mC * T * mU | | |
| WV- | TGUCCAGCUUUAUUGGGAGG | T * fG * mUfC * mCfA * fGfC * mUmU * | XXOXOXOXOXOXOXXXXXX | 1466 |
| 8992 | CTU | mUmA * mUfU * mG * mG * mG * mA * mG * | XXX | |
| | | mG * mC * T * mU | | |
| WV- | TGUCCAGCUUUAUUGGGAGG | T * fG * mUmC * fCfA * fGfC * mUmU * | XXOXOXOXOXOXOXXXXXX | 1467 |
| 8993 | CTU | mUmA * mUfU * mG * mG * mG * mA * mG * | XXX | |
| | | mG * mC * T * mU | | |
| WV- | TGUCCAGCUUUAUUGGGAGG | T * fG * fUfC * fCfA * fGmC * mUmU * mUmA | XXOXOXOXOXOXOXXXXXX | 1468 |
| 8994 | CTU | * mUfU * mG * mG * mG * mA * mG * mG * | XXX | |
| | | mC * T * mU | | |
| WV- | TGUCCAGCUUUAUUGGGAGG | T * fG * fUfC * fCfA * mGfC * mUmU * mUmA | XXOXOXOXOXOXOXXXXXX | 1469 |
| 8995 | CTU | * mUfU * mG * mG * mG * mA * mG * mG * | XXX | |
| | | mC * T * mU | | |
| WV- | TGUCCAGCUUUAUUGGGAGG | T * fG * fUfC * fCmA * fGfC * mUmU * mUmA | XXOXOXOXOXOXOXXXXXX | 1470 |
| 8996 | CTU | * mUfU * mG * mG * mG * mA * mG * mG * | XXX | |
| | | mC * T * mU | | |
| WV- | TGUCCAGCUUUAUUGGGAGG | T * fG * fUfC * mCfA * fGfC * mUmU * mUmA | XXOXOXOXOXOXOXXXXXX | 1471 |
| 8997 | CTU | * mUfU * mG * mG * mG * mA * mG * mG * | XXX | |
| | | mC * T * mU | | |
| WV- | TGUCCAGCUUUAUUGGGAGG | T * fG * fUmC * fCfA * fGfC * mUmU * mUmA | XXOXOXOXOXOXOXXXXXX | 1472 |
| 8998 | CTU | * mUfU * mG * mG * mG * mA * mG * mG * | XXX | |
| | | mC * T * mU | | |
| WV- | TGUCCAGCUUUAUUGGGAGG | T * fG * mUfC * fCfA * fGfC * mUmU * mUmA | XXOXOXOXOXOXOXXXXXX | 1473 |
| 8999 | CTU | * mUfU * mG * mG * mG * mA * mG * mG * | XXX | |
| | | mC * T * mU | | |
| WV- | TGUCCAGCUUUAUUGGGAGG | T * fG * fUfC * fCfA * fGfC * mUmU * mUmA * | XXOXOXOXOXOXOXXXXXX | 1474 |
| 9000 | CTU | mUfU * mG * mG * mG * mA * mG * mG * mC | XXX | |
| | | * T * mU | | |
| WV- | TGUCCAGCUUUAUUGGGAGG | 5MRdT * fG * mUmC * mCmA * mGmC * | XXOXOXOXOXOXOXXXXXX | 1475 |
| 9001 | CTU | mUmU * mUmA * mUfU * mG * mG * mG * | XXX | |
| | | mA * mG * mG * mC * T * mU | | |
| WV- | TAGCUUCUUGUCCAGCUUUA | 5MRdT * fA * mGmC * mUmU * mCmU * | XXOXOXOXOXOXOXXXXXX | 1476 |
| 9002 | UTU | mUmG * mUmC * mCfA * mG * mC * mU * | XXX | |
| | | mU * mU * mA * mU * T * mU | | |
| WV- | TGUCCAGCUUUAUUGGGAGG | 5MRdT * fG * mUmC * mCmA * mGmC * mU * | XXOXOXOXXXOXXXXXXXXX | 1477 |
| 9003 | CTU | mU * mUmA * mU * fU * mG * mG * mG * mA | XX | |
| | | * mG * mG * mC * T * mU | | |
| WV- | TAGCUUCUUGUCCAGCUUUA | 5MRdT * fA * mGmC * mU * mU * mC * mU * | XXOXXXXXXXOXOXXXXXXX | 1478 |
| 9004 | UTU | mU * mG * mUmC * mCfA * mG * mC * mU * | XX | |
| | | mU * mU * mA * mU * T * mU | | |
| WV- | TGUCCAGCUUUAUUGGGAGG | 5MRdT * SfG * SmUmC * SmCmA * SmGmC * | SSOSOSOSOSOSOSSSSSSSS | 1479 |
| 9005 | CTU | SmUmU * SmUmA * SmUfU * SmG * SmG * | S | |
| | | SmG * SmA * SmG * SmG * SmC * ST * SmU | | |
| WV- | TAGCUUCUUGUCCAGCUUUA | 5MRdT * SfA * SmGmC * SmUmU * SmCmU * | SSOSOSOSOSOSOSSSSSSSS | 1480 |
| 9006 | UTU | SmUmG * SmUmC * SmCfA * SmG * SmC * | S | |
| | | SmU * SmU * SmU * SmA * SmU * ST * SmU | | |
| WV- | TGUCCAGCUUUAUUGGGAGG | 5MRdT * SfG * SmUmC * SmCmA * SmGmC * | SSOSOSOSSSOSSSSSSSSSSS | 1481 |
| 9007 | CTU | SmU * SmU * SmUmA * SmU * SfU * SmG * | | |
| | | SmG * SmG * SmA * SmG * SmG * SmC * ST * | | |
| | | SmU | | |
| WV- | TAGCUUCUUGUCCAGCUUUA | 5MRdT * SfA * SmGmC * SmU * SmU * SmC * | SSOSSSSSSSOSOSSSSSSSSS | 1482 |
| 9008 | UTU | SmU * SmU * SmG * SmUmC * SmCfA * SmG * | | |
| | | SmC * SmU * SmU * SmU * SmA * SmU * ST * | | |
| | | SmU | | |
| WV- | TGUCCAGCUUUAUUGGGAGG | PO5MRdT * fG * mUmC * mCmA * mGmC * | XXOXOXOXOXOXOXXXXXX | 1483 |
| 9009 | CTU | mUmU * mUmA * mUfU * mG * mG * mG * | XXX | |
| | | mA * mG * mG * mC * T * mU | | |
| WV- | TAGCUUCUUGUCCAGCUUUA | PO5MRdT * fA * mGmC * mUmU * mCmU * | XXOXOXOXOXOXOXXXXXX | 1484 |
| 9010 | UTU | mUmG * mUmC * mCfA * mG * mC * mU * | XXX | |
| | | mU * mU * mA * mU * T * mU | | |
| WV- | TGUCCAGCUUUAUUGGGAGG | PO5MRdT * fG * mUmC * mCmA * mGmC * | XXOXOXOXXXOXXXXXXXXX | 1485 |
| 9011 | CTU | mU * mU * mUmA * mU * fU * mG * mG * mG | XX | |
| | | * mA * mG * mG * mC * T * mU | | |
| WV- | TAGCUUCUUGUCCAGCUUUA | PO5MRdT * fA * mGmC * mU * mU * mC * | XXOXXXXXXXOXOXXXXXXX | 1486 |
| 9012 | UTU | mU * mU * mG * mUmC * mCfA * mG * mC * | XX | |
| | | mU * mU * mU * mA * mU * T * mU | | |
| WV- | TGUCCAGCUUUAUUGGGAGG | PO5MRdT * SfG * SmUmC * SmCmA * SmGmC | SSOSOSOSOSOSOSSSSSSSS | 1487 |
| 9013 | CTU | * SmUmU * SmUmA * SmUfU * SmG * SmG * | S | |
| | | SmG * SmA * SmG * SmG * SmC * ST * SmU | | |
| WV- | TAGCUUCUUGUCCAGCUUUA | PO5MRdT * SfA * SmGmC * SmUmU * SmCmU | SSOSOSOSOSOSOSSSSSSSS | 1488 |
| 9014 | UTU | * SmUmG * SmUmC * SmCfA * SmG * SmC * | S | |
| | | SmU * SmU * SmU * SmA * SmU * ST * SmU | | |
| WV- | TGUCCAGCUUUAUUGGGAGG | PO5MRdT * SfG * SmUmC * SmCmA * SmGmC | SSOSOSOSSSOSSSSSSSSSSS | 1489 |
| 9015 | CTU | * SmU * SmU * SmUmA * SmU * SfU * SmG * | | |
| | | SmG * SmG * SmA * SmG * SmG * SmC * ST * | | |
| | | SmU | | |
| WV- | TAGCUUCUUGUCCAGCUUUA | PO5MRdT * SfA * SmGmC * SmU * SmU * SmC | SSOSSSSSSSOSOSSSSSSSSS | 1490 |
| 9016 | UTU | * SmU * SmU * SmG * SmUmC * SmCfA * SmG | | |
| | | * SmC * SmU * SmU * SmU * SmA * SmU * ST | | |
| | | * SmU | | |
| WV- | TGUCCAGCUUUAUUGGGAGG | PS5MRdT * fG * mUmC * mCmA * mGmC * | XXOXOXOXOXOXOXXXXXX | 1491 |
| 9017 | CTU | mUmU * mUmA * mUfU * mG * mG * mG * | XXX | |
| | | mA * mG * mG * mC * T * mU | | |
| WV- | TAGCUUCUUGUCCAGCUUUA | PS5MRdT * fA * mGmC * mUmU * mCmU * | XXOXOXOXOXOXOXXXXXX | 1492 |
| 9018 | UTU | mUmG * mUmC * mCfA * mG * mC * mU * | XXX | |
| | | mU * mU * mA * mU * T * mU | | |
| WV- | TGUCCAGCUUUAUUGGGAGG | PS5MRdT * fG * mUmC * mCmA * mGmC * | XXOXOXOXXXOXXXXXXXXX | 1493 |
| 9019 | CTU | mU * mU * mUmA * mU * fU * mG * mG * mG | XX | |
| | | * mA * mG * mG * mC * T * mU | | |
| WV- | TAGCUUCUUGUCCAGCUUUA | PS5MRdT * fA * mGmC * mU * mU * mC * mU | XXOXXXXXXXOXOXXXXXXX | 1494 |
| 9020 | UTU | * mU * mG * mUmC * mCfA * mG * mC * mU | XX | |
| | | * mU * mU * mA * mU * T * mU | | |
| WV- | TGUCCAGCUUUAUUGGGAGG | PS5MRdT * SfG * SmUmC * SmCmA * SmGmC | SSOSOSOSOSOSOSSSSSSSS | 1495 |
| 9021 | CTU | * SmUmU * SmUmA * SmUfU * SmG * SmG * | S | |
| | | SmG * SmA * SmG * SmG * SmC * ST * SmU | | |
| WV- | TAGCUUCUUGUCCAGCUUUA | PS5MRdT * SfA * SmGmC * SmUmU * SmCmU | SSOSOSOSOSOSOSSSSSSSS | 1496 |
| 9022 | UTU | * SmUmG * SmUmC * SmCfA * SmG * SmC * | S | |
| | | SmU * SmU * SmU * SmA * SmU * ST * SmU | | |
| WV- | TGUCCAGCUUUAUUGGGAGG | PS5MRdT * SfG * SmUmC * SmCmA * SmGmC | SSOSOSOSSSOSSSSSSSSSSS | 1497 |
| 9023 | CTU | * SmU * SmU * SmUmA * SmU * SfU * SmG * | | |
| | | SmG * SmG * SmA * SmG * SmG * SmC * ST * | | |
| | | SmU | | |
| WV- | TAGCUUCUUGUCCAGCUUUA | PS5MRdT * SfA * SmGmC * SmU * SmU * SmC | SSOSSSSSSSOSOSSSSSSSSS | 1498 |
| 9024 | UTU | * SmU * SmU * SmG * SmUmC * SmCfA * SmG | | |
| | | * SmC * SmU * SmU * SmU * SmA * SmU * ST | | |
| | | * SmU | | |
| WV- | TAGCUUCUUGUCCAGCUUUA | T * fA * mGfC * mUfU * mCfU * mUmG * | XXOXOXOXOXOXOXXXXXX | 1499 |
| 9025 | UTU | mUmC * mCfA * mG * mC * mU * mU * mU * | XXX | |
| | | mA * mU * T * mU | | |
| WV- | TGUCCAGCUUUAUUGGGAGG | T * fG * mUfC * mCfA * mGfC * mU * mU * | XXOXOXOXXXOXXXXXXXXX | 1500 |
| 9026 | CTU | mUmA * mU * fU * mG * mG * mG * mA * mG | XX | |
| | | * mG * mC * T * mU | | |
| WV- | TAGCUUCUUGUCCAGCUUUA | T * fA * mGfC * mU * fU * mC * fU * mU * mG | XXOXXXXXXXOXOXXXXXXX | 1501 |
| 9027 | UTU | * mUmC * mCfA * mG * mC * mU * mU * mU | XX | |
| | | * mA * mU * T * mU | | |
| WV- | TGUCCAGCUUUAUUGGGAGG | T * SfG * SmUfC * SmCfA * SmGfC * SmUmU * | SSOSOSOSOSOSOSSSSSSSS | 1502 |
| 9028 | CTU | SmUmA * SmUfU * SmG * SmG * SmG * SmA | S | |
| | | * SmG * SmG * SmC * ST * SmU | | |
| WV- | TAGCUUCUUGUCCAGCUUUA | T * SfA * SmGfC * SmUfU * SmCfU * SmUmG * | SSOSOSOSOSOSOSSSSSSSS | 1503 |
| 9029 | UTU | SmUmC * SmCfA * SmG * SmC * SmU * SmU * | S | |
| | | SmU * SmA * SmU * ST * SmU | | |
| WV- | TGUCCAGCUUUAUUGGGAGG | T * SfG * SmUfC * SmCfA * SmGfC * SmU * | SSOSOSOSSSOSSSSSSSSSSS | 1504 |
| 9030 | CTU | SmU * SmUmA * SmU * SfU * SmG * SmG * | | |
| | | SmG * SmA * SmG * SmG * SmC * ST * SmU | | |
| WV- | TAGCUUCUUGUCCAGCUUUA | T * SfA * SmGfC * SmU * SfU * SmC * SfU * | SSOSSSSSSSOSOSSSSSSSSS | 1505 |
| 9031 | UTU | SmU * SmG * SmUmC * SmCfA * SmG * SmC * | | |
| | | SmU * SmU * SmU * SmA * SmU * ST * SmU | | |
| WV- | TGUCCAGCUUUAUUGGGAGG | 5MRdT * fG * mUfC * mCfA * mGfC * mUmU * | XXOXOXOXOXOXOXXXXXX | 1506 |
| 9032 | CTU | mUmA * mUfU * mG * mG * mG * mA * mG * | XXX | |
| | | mG * mC * T * mU | | |
| WV- | TAGCUUCUUGUCCAGCUUUA | 5MRdT * fA * mGfC * mUfU * mCfU * mUmG | XXOXOXOXOXOXOXXXXXX | 1507 |
| 9033 | UTU | * mUmC * mCfA * mG * mC * mU * mU * mU | XXX | |
| | | * mA * mU * T * mU | | |
| WV- | TGUCCAGCUUUAUUGGGAGG | 5MRdT * fG * mUfC * mCfA * mGfC * mU * | XXOXOXOXXXOXXXXXXXXX | 1508 |
| 9034 | CTU | mU * mUmA * mU * fU * mG * mG * mG * mA | XX | |
| | | * mG * mG * mC * T * mU | | |
| WV- | TAGCUUCUUGUCCAGCUUUA | 5MRdT * fA * mGfC * mU * fU * mC * fU * mU | XXOXXXXXXXOXOXXXXXXX | 1509 |
| 9035 | UTU | * mG * mUmC * mCfA * mG * mC * mU * mU | XX | |
| | | * mU * mA * mU * T * mU | | |
| WV- | TGUCCAGCUUUAUUGGGAGG | 5MRdT * SfG * SmUfC * SmCfA * SmGfC * | SSOSOSOSOSOSOSSSSSSSS | 1510 |
| 9036 | CTU | SmUmU * SmUmA * SmUfU * SmG * SmG * | S | |
| | | SmG * SmA * SmG * SmG * SmC * ST * SmU | | |
| WV- | TAGCUUCUUGUCCAGCUUUA | 5MRdT * SfA * SmGfC * SmUfU * SmCfU * | SSOSOSOSOSOSOSSSSSSSS | 1511 |
| 9037 | UTU | SmUmG * SmUmC * SmCfA * SmG * SmC * | S | |
| | | SmU * SmU * SmU * SmA * SmU * ST * SmU | | |
| WV- | TGUCCAGCUUUAUUGGGAGG | 5MRdT * SfG * SmUfC * SmCfA * SmGfC * | SSOSOSOSSSOSSSSSSSSSSS | 1512 |
| 9038 | CTU | SmU * SmU * SmUmA * SmU * SfU * SmG * | | |
| | | SmG * SmG * SmA * SmG * SmG * SmC * ST * | | |
| | | SmU | | |
| WV- | TAGCUUCUUGUCCAGCUUUA | 5MRdT * SfA * SmGfC * SmU * SfU * SmC * | SSOSSSSSSSOSOSSSSSSSSS | 1513 |
| 9039 | UTU | SfU * SmU * SmG * SmUmC * SmCfA * SmG * | | |
| | | SmC * SmU * SmU * SmU * SmA * SmU * ST * | | |
| | | SmU | | |
| WV- | TGUCCAGCUUUAUUGGGAGG | PO5MRdT * fG * mUfC * mCfA * mGfC * | XXOXOXOXOXOXOXXXXXX | 1514 |
| 9040 | CTU | mUmU * mUmA * mUfU * mG * mG * mG * | XXX | |
| | | mA * mG * mG * mC * T * mU | | |
| WV- | TAGCUUCUUGUCCAGCUUUA | PO5MRdT * fA * mGfC * mUfU * mCfU * | XXOXOXOXOXOXOXXXXXX | 1515 |
| 9041 | UTU | mUmG * mUmC * mCfA * mG * mC * mU * | XXX | |
| | | mU * mU * mA * mU * T * mU | | |
| WV- | TGUCCAGCUUUAUUGGGAGG | PO5MRdT * fG * mUfC * mCfA * mGfC * mU * | XXOXOXOXXXOXXXXXXXXX | 1516 |
| 9042 | CTU | mU * mUmA * mU * fU * mG * mG * mG * mA | XX | |
| | | * mG * mG * mC * T * mU | | |
| WV- | TAGCUUCUUGUCCAGCUUUA | PO5MRdT * fA * mGfC * mU * fU * mC * fU * | XXOXXXXXXXOXOXXXXXXX | 1517 |
| 9043 | UTU | mU * mG * mUmC * mCfA * mG * mC * mU * | XX | |
| | | mU * mU * mA * mU * T * mU | | |
| WV- | TGUCCAGCUUUAUUGGGAGG | PO5MRdT * SfG * SmUfC * SmCfA * SmGfC * | SSOSOSOSOSOSOSSSSSSSS | 1518 |
| 9044 | CTU | SmUmU * SmUmA * SmUfU * SmG * SmG * | S | |
| | | SmG * SmA * SmG * SmG * SmC * ST * SmU | | |
| WV- | TAGCUUCUUGUCCAGCUUUA | PO5MRdT * SfA * SmGfC * SmUfU * SmCfU * | SSOSOSOSOSOSOSSSSSSSS | 1519 |
| 9045 | UTU | SmUmG * SmUmC * SmCfA * SmG * SmC * | S | |
| | | SmU * SmU * SmU * SmA * SmU * ST * SmU | | |
| WV- | TGUCCAGCUUUAUUGGGAGG | PO5MRdT * SfG * SmUfC * SmCfA * SmGfC * | SSOSOSOSSSOSSSSSSSSSSS | 1520 |
| 9046 | CTU | SmU * SmU * SmUmA * SmU * SfU * SmG * | | |
| | | SmG * SmG * SmA * SmG * SmG * SmC * ST * | | |
| | | SmU | | |
| WV- | TAGCUUCUUGUCCAGCUUUA | PO5MRdT * SfA * SmGfC * SmU * SfU * SmC * | SSOSSSSSSSOSOSSSSSSSSS | 1521 |
| 9047 | UTU | SfU * SmU * SmG * SmUmC * SmCfA * SmG * | | |
| | | SmC * SmU * SmU * SmU * SmA * SmU * ST * | | |
| | | SmU | | |
| WV- | TGUCCAGCUUUAUUGGGAGG | PS5MRdT * fG * mUfC * mCfA * mGfC * | XXOXOXOXOXOXOXXXXXX | 1522 |
| 9048 | CTU | mUmU * mUmA * mUfU * mG * mG * mG * | XXX | |
| | | mA * mG * mG * mC * T * mU | | |
| WV- | TAGCUUCUUGUCCAGCUUUA | PS5MRdT * fA * mGfC * mUfU * mCfU * | XXOXOXOXOXOXOXXXXXX | 1523 |
| 9049 | UTU | mUmG * mUmC * mCfA * mG * mC * mU * | XXX | |
| | | mU * mU * mA * mU * T * mU | | |
| WV- | TGUCCAGCUUUAUUGGGAGG | PS5MRdT * fG * mUfC * mCfA * mGfC * mU * | XXOXOXOXXXOXXXXXXXXX | 1524 |
| 9050 | CTU | mU * mUmA * mU * fU * mG * mG * mG * mA | XX | |
| | | * mG * mG * mC * T * mU | | |
| WV- | TAGCUUCUUGUCCAGCUUUA | PS5MRdT * fA * mGfC * mU * fU * mC * fU * | XXOXXXXXXXOXOXXXXXXX | 1525 |
| 9051 | UTU | mU * mG * mUmC * mCfA * mG * mC * mU * | XX | |
| | | mU * mU * mA * mU * T * mU | | |
| WV- | TGUCCAGCUUUAUUGGGAGG | PS5MRdT * SfG * SmUfC * SmCfA * SmGfC * | SSOSOSOSOSOSOSSSSSSSS | 1526 |
| 9052 | CTU | SmUmU * SmUmA * SmUfU * SmG * SmG * | S | |
| | | SmG * SmA * SmG * SmG * SmC * ST * SmU | | |
| WV- | TAGCUUCUUGUCCAGCUUUA | PS5MRdT * SfA * SmGfC * SmUfU * SmCfU * | SSOSOSOSOSOSOSSSSSSSS | 1527 |
| 9053 | UTU | SmUmG * SmUmC * SmCfA * SmG * SmC * | S | |
| | | SmU * SmU * SmU * SmA * SmU * ST * SmU | | |
| WV- | TGUCCAGCUUUAUUGGGAGG | PS5MRdT * SfG * SmUfC * SmCfA * SmGfC * | SSOSOSOSSSOSSSSSSSSSSS | 1528 |
| 9054 | CTU | SmU * SmU * SmUmA * SmU * SfU * SmG * | | |
| | | SmG * SmG * SmA * SmG * SmG * SmC * ST * | | |
| | | SmU | | |
| WV- | TAGCUUCUUGUCCAGCUUUA | PS5MRdT * SfA * SmGfC * SmU * SfU * SmC * | SSOSSSSSSSOSOSSSSSSSSS | 1529 |
| 9055 | UTU | SfU * SmU * SmG * SmUmC * SmCfA * SmG * | | |
| | | SmC * SmU * SmU * SmU * SmA * SmU * ST * | | |
| | | SmU | | |
| WV- | TGUCCAGCUUUAUUGGGAGG | L001T * SfG * SmUfC * SmCfA * SmGfC * SmU | OSSOSOSOSSSOSSSSSSSSS | 1530 |
| 9211 | CTU | * SfU * SmUfA * SmU * SfU * SmG * SfG * | SS | |
| | | SmG * SfA * SmG * SfG * SmC * ST * SmU | | |
| WV- | TGUCCAGCUUUAUUGGGAGG | L0015MRdT * SfG * SmUfC * SmCfA * SmGfC * | OSSOSOSOSSSOSSSSSSSSS | 1531 |
| 9212 | CTU | SmU * SfU * SmUfA * SmU * SfU * SmG * SfG SS | | |
| | | * SmG * SfA * SmG * SfG * SmC * ST * SmU | | |
| WV- | TGUCCAGCUUUAUUGGGAGG | POT * SfG * SmUfC * SmCfA * SmGfC * SmU * | SSOSOSOSSSOSSSSSSSSSSS | 1532 |
| 9213 | CTU | SfU * SmUfA * SmU * SfU * SmG * SfG * SmG | | |
| | | * SfA * SmG * SfG * SmC * SAMC6T * SmU | | |
| WV- | TGUCCAGCUUUAUUGGGAGG | T * SfG * SmUmC * SmCmA * SmGmC * SmU * | SSOSOSOSSSOSSSSSSSSSSS | 1533 |
| 9214 | CTU | SmU * SmUmA * SmU * SfU * SmG * SmG * | | |
| | | SmG * SmA * SmG * SmG * SmC * STGaNC6T * | | |
| | | SmU | | |
| WV- | TGUCCAGCUUUAUUGGGAGG | 5MRdT * SfG * SmUmC * SmCmA * SmGmC * | SSOSOSOSSSOSSSSSSSSSSS | 1534 |
| 9215 | CTU | SmU * SmU * SmUmA * SmU * SfU * SmG * | | |
| | | SmG * SmG * SmA * SmG * SmG * SmC * | | |
| | | STGaNC6T * SmU | | |
| WV- | TGUCCAGCUUUAUUGGGAGG | PO5MRdT * SfG * SmUmC * SmCmA * SmGmC | SSOSOSOSSSOSSSSSSSSSSS | 1535 |
| 9216 | CTU | * SmU * SmU * SmUmA * SmU * SfU * SmG * | | |
| | | SmG * SmG * SmA * SmG * SmG * SmC * | | |
| | | STGaNC6T * SmU | | |
| WV- | TGUCCAGCUUUAUUGGGAGG | PS5MRdT * SfG * SmUmC * SmCmA * SmGmC | SSOSOSOSSSOSSSSSSSSSSS | 1536 |
| 9217 | CTU | * SmU * SmU * SmUmA * SmU * SfU * SmG * | | |
| | | SmG * SmG * SmA * SmG * SmG * SmC * | | |
| | | STGaNC6T * SmU | | |
| WV- | TGUCCAGCUUUAUUGGGAGG | T * SfG * SmUfC * SmCfA * SmGfC * SmU * | SSOSOSOSSSOSSSSSSSSSSS | 1537 |
| 9218 | CTU | SmU * SmUmA * SmU * SfU * SmG * SmG * | | |
| | | SmG * SmA * SmG * SmG * SmC * STGaNC6T * | | |
| | | SmU | | |
| WV- | TGUCCAGCUUUAUUGGGAGG | 5MRdT * SfG * SmUfC * SmCfA * SmGfC * | SSOSOSOSSSOSSSSSSSSSSS | 1538 |
| 9219 | CTU | SmU * SmU * SmUmA * SmU * SfU * SmG * | | |
| | | SmG * SmG * SmA * SmG * SmG * SmC * | | |
| | | STGaNC6T * SmU | | |
| WV- | TGUCCAGCUUUAUUGGGAGG | PO5MRdT * SfG * SmUfC * SmCfA * SmGfC * | SSOSOSOSSSOSSSSSSSSSSS | 1539 |
| 9220 | CTU | SmU * SmU * SmUmA * SmU * SfU * SmG * | | |
| | | SmG * SmG * SmA * SmG * SmG * SmC * | | |
| | | STGaNC6T * SmU | | |
| WV- | TGUCCAGCUUUAUUGGGAGG | PS5MRdT * SfG * SmUfC * SmCfA * SmGfC * | SSOSOSOSSSOSSSSSSSSSSS | 1540 |
| 9221 | CTU | SmU * SmU * SmUmA * SmU * SfU * SmG * | | |
| | | SmG * SmG * SmA * SmG * SmG * SmC * | | |
| | | STGaNC6T * SmU | | |
| WV- | TGUCCAGCUUUAUUGGGAGG | T * SfG * SmUmC * SmCmA * SmGmC * SmU * | SSOSOSOSSSOSSSSSSSSSSS | 1541 |
| 9229 | CTU | SmU * SmUmA * SmU * SfU * SmG * SmG * | | |
| | | SmG * SmA * SmG * SmG * SmC * SAMC6T * | | |
| | | SmU | | |
| WV- | TGUCCAGCUUUAUUGGGAGG | 5MRdT * SfG * SmUmC * SmCmA * SmGmC * | SSOSOSOSSSOSSSSSSSSSSS | 1542 |
| 9230 | CTU | SmU * SmU * SmUmA * SmU * SfU * SmG * | | |
| | | SmG * SmG * SmA * SmG * SmG * SmC * | | |
| | | SAMC6T * SmU | | |
| WV- | TGUCCAGCUUUAUUGGGAGG | PO5MRdT * SfG * SmUmC * SmCmA * SmGmC | SSOSOSOSSSOSSSSSSSSSSS | 1543 |
| 9231 | CTU | * SmU * SmU * SmUmA * SmU * SfU * SmG * | | |
| | | SmG * SmG * SmA * SmG * SmG * SmC * | | |
| | | SAMC6T * SmU | | |
| WV- | TGUCCAGCUUUAUUGGGAGG | PS5MRdT * SfG * SmUmC * SmCmA * SmGmC | SSOSOSOSSSOSSSSSSSSSSS | 1544 |
| 9232 | CTU | * SmU * SmU * SmUmA * SmU * SfU * SmG * | | |
| | | SmG * SmG * SmA * SmG * SmG * SmC * | | |
| | | SAMC6T * SmU | | |
| WV- | TGUCCAGCUUUAUUGGGAGG | T * SfG * SmUfC * SmCfA * SmGfC * SmU * | SSOSOSOSSSOSSSSSSSSSSS | 1545 |
| 9233 | CTU | SmU * SmUmA * SmU * SfU * SmG * SmG * | | |
| | | SmG * SmA * SmG * SmG * SmC * SAMC6T * | | |
| | | SmU | | |
| WV- | TGUCCAGCUUUAUUGGGAGG | 5MRdT * SfG * SmUfC * SmCfA * SmGfC * | SSOSOSOSSSOSSSSSSSSSSS | 1546 |
| 9234 | CTU | SmU * SmU * SmUmA * SmU * SfU * SmG * | | |
| | | SmG * SmG * SmA * SmG * SmG * SmC * | | |
| | | SAMC6T * SmU | | |
| WV- | TGUCCAGCUUUAUUGGGAGG | PO5MRdT * SfG * SmUfC * SmCfA * SmGfC * | SSOSOSOSSSOSSSSSSSSSSS | 1547 |
| 9235 | CTU | SmU * SmU * SmUmA * SmU * SfU * SmG * | | |
| | | SmG * SmG * SmA * SmG * SmG * SmC * | | |
| | | SAMC6T * SmU | | |
| WV- | TGUCCAGCUUUAUUGGGAGG | PS5MRdT * SfG * SmUfC * SmCfA * SmGfC * | SSOSOSOSSSOSSSSSSSSSSS | 1548 |
| 9236 | CTU | SmU * SmU * SmUmA * SmU * SfU * SmG * | | |
| | | SmG * SmG * SmA * SmG * SmG * SmC * | | |
| | | SAMC6T * SmU | | |
| WV- | TGUCCAGCUUUAUUGGGAGG | PO5MRdT * SfG * SmUfC * SmCfA * SmGfC * | SSOSOSOSXSOSSSSSSSSSSS | 1549 |
| 9237 | CTU | Spac3mU * fU * SmUfA * SmU * SfU * SmG * | | |
| | | SfG * SmG * SfA * SmG * SfG * SmC * SAMC6T | | |
| | | * SmU | | |
| WV- | TGUCCAGCUUUAUUGGGAGG | PO5MRdT * SfG * SmUfC * SmCfA * SmGfC * | SSOSOSOSSSOSSSSSSSSSSS | 1550 |
| 9238 | CTU | SmU * SfU * SmUfA * SmU * SfU * SmG * SfG | | |
| | | * SmG * SfA * SmG * SfG * SmC * SAMC6T * | | |
| | | Spac3mU | | |
| WV- | GUCCAGCUUUAUUGGGAGGC | L009 * fG * mUfC * mCfA * mGfC * mUfU * | XXOXOXOXOXOXOXXXXXX | 1551 |
| 9239 | TU | mUfA * mUfU * mG * fG * mG * fA * mG * fG | XXX | |
| | | * mC * T * mU | | |
| WV- | TUCCAGCUUUAUUGGGAGGC | T * L009 * mUfC * mCfA * mGfC * mUfU * | XXOXOXOXOXOXOXXXXXX | 1552 |
| 9240 | TU | mUfA * mUfU * mG * fG * mG * fA * mG * fG | XXX | |
| | | * mC * T * mU | | |
| WV- | TGCCAGCUUUAUUGGGAGGC | T * fG * L009fC * mCfA * mGfC * mUfU * | XXOXOXOXOXOXOXXXXXX | 1553 |
| 9241 | TU | mUfA * mUfU * mG * fG * mG * fA * mG * fG | XXX | |
| | | * mC * T * mU | | |
| WV- | TGUCAGCUUUAUUGGGAGGC | T * fG * mUL009 * mCfA * mGfC * mUfU * | XXOXOXOXOXOXOXXXXXX | 1554 |
| 9242 | TU | mUfA * mUfU * mG * fG * mG * fA * mG * fG | XXX | |
| | | * mC * T * mU | | |
| WV- | TGUCAGCUUUAUUGGGAGGC | T * fG * mUfC * L009fA * mGfC * mUfU * | XXOXOXOXOXOXOXXXXXX | 1555 |
| 9243 | TU | mUfA * mUfU * mG * fG * mG * fA * mG * fG | XXX | |
| | | * mC * T * mU | | |
| WV- | TGUCCGCUUUAUUGGGAGGC | T * fG * mUfC * mCL009 * mGfC * mUfU * | XXOXOXOXOXOXOXXXXXX | 1556 |
| 9244 | TU | mUfA * mUfU * mG * fG * mG * fA * mG * fG | XXX | |
| | | * mC * T * mU | | |
| WV- | TGUCCACUUUAUUGGGAGGC | T * fG * mUfC * mCfA * L009fC * mUfU * | XXOXOXOXOXOXOXXXXXX | 1557 |
| 9245 | TU | mUfA * mUfU * mG * fG * mG * fA * mG * fG | XXX | |
| | | * mC * T * mU | | |
| WV- | TGUCCAGUUUAUUGGGAGGC | T * fG * mUfC * mCfA * mGL009 * mUfU * | XXOXOXOXOXOXOXXXXXX | 1558 |
| 9246 | TU | mUfA * mUfU * mG * fG * mG * fA * mG * fG | XXX | |
| | | * mC * T * mU | | |
| WV- | TGUCCAGCUUAUUGGGAGGC | T * fG * mUfC * mCfA * mGfC * L009fU * | XXOXOXOXOXOXOXXXXXX | 1559 |
| 9247 | TU | mUfA * mUfU * mG * fG * mG * fA * mG * fG | XXX | |
| | | * mC * T * mU | | |
| WV- | TGUCCAGCUUAUUGGGAGGC | T * fG * mUfC * mCfA * mGfC * mUL009 * | XXOXOXOXOXOXOXXXXXX | 1560 |
| 9248 | TU | mUfA * mUfU * mG * fG * mG * fA * mG * fG | XXX | |
| | | * mC * T * mU | | |
| WV- | TGUCCAGCUUAUUGGGAGGC | T * fG * mUfC * mCfA * mGfC * mUfU * | XXOXOXOXOXOXOXXXXXX | 1561 |
| 9249 | TU | L009fA * mUfU * mG * fG * mG * fA * mG * fG | XXX | |
| | | * mC * T * mU | | |
| WV- | GUCCAGCUUUAUUGGGAGGC | L010 * fG * mUfC * mCfA * mGfC * mUfU * | XXOXOXOXOXOXOXXXXXX | 1562 |
| 9250 | TU | mUfA * mUfU * mG * fG * mG * fA * mG * fG | XXX | |
| | | * mC * T * mU | | |
| WV- | TUCCAGCUUUAUUGGGAGGC | T * L010 * mUfC * mCfA * mGfC * mUfU * | XXOXOXOXOXOXOXXXXXX | 1563 |
| 9251 | TU | mUfA * mUfU * mG * fG * mG * fA * mG * fG | XXX | |
| | | * mC * T * mU | | |
| WV- | TGCCAGCUUUAUUGGGAGGC | T * fG * L010fC * mCfA * mGfC * mUfU * | XXOXOXOXOXOXOXXXXXX | 1564 |
| 9252 | TU | mUfA * mUfU * mG * fG * mG * fA * mG * fG | XXX | |
| | | * mC * T * mU | | |
| WV- | TGUCAGCUUUAUUGGGAGGC | T * fG * mUL010 * mCfA * mGfC * mUfU * | XXOXOXOXOXOXOXXXXXX | 1565 |
| 9253 | TU | mUfA * mUfU * mG * fG * mG * fA * mG * fG | XXX | |
| | | * mC * T * mU | | |
| WV- | TGUCAGCUUUAUUGGGAGGC | T * fG * mUfC * L010fA * mGfC * mUfU * | XXOXOXOXOXOXOXXXXXX | 1566 |
| 9254 | TU | mUfA * mUfU * mG * fG * mG * fA * mG * fG | XXX | |
| | | * mC * T * mU | | |
| WV- | TGUCCGCUUUAUUGGGAGGC | T * fG * mUfC * mCL010 * mGfC * mUfU * | XXOXOXOXOXOXOXXXXXX | 1567 |
| 9255 | TU | mUfA * mUfU * mG * fG * mG * fA * mG * fG | XXX | |
| | | * mC * T * mU | | |
| WV- | TGUCCACUUUAUUGGGAGGC | T * fG * mUfC * mCfA * L010fC * mUfU * | XXOXOXOXOXOXOXXXXXX | 1568 |
| 9256 | TU | mUfA * mUfU * mG * fG * mG * fA * mG * fG | XXX | |
| | | * mC * T * mU | | |
| WV- | TGUCCAGUUUAUUGGGAGGC | T * fG * mUfC * mCfA * mGL010 * mUfU * | XXOXOXOXOXOXOXXXXXX | 1569 |
| 9257 | TU | mUfA * mUfU * mG * fG * mG * fA * mG * fG | XXX | |
| | | * mC * T * mU | | |
| WV- | TGUCCAGCUUAUUGGGAGGC | T * fG * mUfC * mCfA * mGfC * L010fU * | XXOXOXOXOXOXOXXXXXX | 1570 |
| 9258 | TU | mUfA * mUfU * mG * fG * mG * fA * mG * fG | XXX | |
| | | * mC * T * mU | | |
| WV- | TGUCCAGCUUAUUGGGAGGC | T * fG * mUfC * mCfA * mGfC * mUL010 * | XXOXOXOXOXOXOXXXXXX | 1571 |
| 9259 | TU | mUfA * mUfU * mG * fG * mG * fA * mG * fG | XXX | |
| | | * mC * T * mU | | |
| WV- | TGUCCAGCUUAUUGGGAGGC | T * fG * mUfC * mCfA * mGfC * mUfU * | XXOXOXOXOXOXOXXXXXX | 1572 |
| 9260 | TU | L010fA * mUfU * mG * fG * mG * fA * mG * fG | XXX | |
| | | * mC * T * mU | | |
| WV- | GCCACUGUAGAAAGGCAUGA | L009 * fG * mCfC * mAfC * mUfG * mUfA * | XXOXOXOXOXOXOXOOOO | 1573 |
| 9261 | TU | mGfA * mAfA * mGfGmCfAmUfGmA * T * mU | OOXX | |
| WV- | TCCACUGUAGAAAGGCAUGAT | T * L009 * mCfC * mAfC * mUfG * mUfA * | XXOXOXOXOXOXOXOOOO | 1574 |
| 9262 | U | mGfA * mAfA * mGfGmCfAmUfGmA * T * mU | OOXX | |
| WV- | TGCACUGUAGAAAGGCAUGAT | T * fG * L009fC * mAfC * mUfG * mUfA * | XXOXOXOXOXOXOXOOOO | 1575 |
| 9263 | U | mGfA * mAfA * mGfGmCfAmUfGmA * T * mU | OOXX | |
| WV- | TGCACUGUAGAAAGGCAUGAT | T * fG * mCL009 * mAfC * mUfG * mUfA * | XXOXOXOXOXOXOXOOOO | 1576 |
| 9264 | U | mGfA * mAfA * mGfGmCfAmUfGmA * T * mU | OOXX | |
| WV- | TGCCCUGUAGAAAGGCAUGAT | T * fG * mCfC * L009fC * mUfG * mUfA * | XXOXOXOXOXOXOXOOOO | 1577 |
| 9265 | U | mGfA * mAfA * mGfGmCfAmUfGmA * T * mU | OOXX | |
| WV- | TGCCAUGUAGAAAGGCAUGAT | T * fG * mCfC * mAL009 * mUfG * mUfA * | XXOXOXOXOXOXOXOOOO | 1578 |
| 9266 | U | mGfA * mAfA * mGfGmCfAmUfGmA * T * mU | OOXX | |
| WV- | TGCCACGUAGAAAGGCAUGAT | T * fG * mCfC * mAfC * LOO9fG * mUfA * mGfA | XXOXOXOXOXOXOXOOOO | 1579 |
| 9267 | U | * mAfA * mGfGmCfAmUfGmA * T * mU | OOXX | |
| WV- | TGCCACUUAGAAAGGCAUGAT | T * fG * mCfC * mAfC * mU L009 * mUfA * | XXOXOXOXOXOXOXOOOO | 1580 |
| 9268 | U | mGfA * mAfA * mGfGmCfAmUfGmA * T * mU | OOXX | |
| WV- | TGCCACUGAGAAAGGCAUGAT | T * fG * mCfC * mAfC * mUfG * L009fA * mGfA | XXOXOXOXOXOXOXOOOO | 1581 |
| 9269 | U | * mAfA * mGfGmCfAmUfGmA * T * mU | OOXX | |
| WV- | TGCCACUGUGAAAGGCAUGAT | T * fG * mCfC * mAfC * mUfG * mUL009 * | XXOXOXOXOXOXOXOOOO | 1582 |
| 9270 | U | mGfA * mAfA * mGfGmCfAmUfGmA * T * mU | OOXX | |
| WV- | TGCCACUGUAAAAGGCAUGAT | T * fG * mCfC * mAfC * mUfG * mUfA * L009fA | XXOXOXOXOXOXOXOOOO | 1583 |
| 9271 | U | * mAfA * mGfGmCfAmUfGmA * T * mU | OOXX | |
| WV- | TGCCACUGUAGAAGGCAUGAT | T * fG * mCfC * mAfC * mUfG * mUfA * mGfA | XXOXOXOXOXOXOXOOOO | 1584 |
| 9272 | U | * mAL009 * mGfGmCfAmUfGmA * T * mU | OOXX | |
| WV- | GCCACUGUAGAAAGGCAUGA | L010 * fG * mCfC * mAfC * mUfG * mUfA * | XXOXOXOXOXOXOXOOOO | 1585 |
| 9273 | TU | mGfA * mAfA * mGfGmCfAmUfGmA * T * mU | OOXX | |
| WV- | TCCACUGUAGAAAGGCAUGAT | T * L010 * mCfC * mAfC * mUfG * mUfA * | XXOXOXOXOXOXOXOOOO | 1586 |
| 9274 | U | mGfA * mAfA * mGfGmCfAmUfGmA * T * mU | OOXX | |
| WV- | TGCACUGUAGAAAGGCAUGAT | T * fG * L010fC * mAfC * mUfG * mUfA * | XXOXOXOXOXOXOXOOOO | 1587 |
| 9275 | U | mGfA * mAfA * mGfGmCfAmUfGmA * T * mU | OOXX | |
| WV- | TGCACUGUAGAAAGGCAUGAT | T * fG * mCL010 * mAfC * mUfG * mUfA * | XXOXOXOXOXOXOXOOOO | 1588 |
| 9276 | U | mGfA * mAfA * mGfGmCfAmUfGmA * T * mU | OOXX | |
| WV- | TGCCCUGUAGAAAGGCAUGAT | T * fG * mCfC * L010fC * mUfG * mUfA * | XXOXOXOXOXOXOXOOOO | 1589 |
| 9277 | U | mGfA * mAfA * mGfGmCfAmUfGmA * T * mU | OOXX | |
| WV- | TGCCAUGUAGAAAGGCAUGAT | T * fG * mCfC * mAL010 * mUfG * mUfA * | XXOXOXOXOXOXOXOOOO | 1590 |
| 9278 | U | mGfA * mAfA * mGfGmCfAmUfGmA * T * mU | OOXX | |
| WV- | TGCCACGUAGAAAGGCAUGAT | T * fG * mCfC * mAfC * L010fG * mUfA * mGfA | XXOXOXOXOXOXOXOOOO | 1591 |
| 9279 | U | * mAfA * mGfGmCfAmUfGmA * T * mU | OOXX | |
| WV- | TGCCACUUAGAAAGGCAUGAT | T * fG * mCfC * mAfC * mU L010 * mUfA * | XXOXOXOXOXOXOXOOOO | 1592 |
| 9280 | U | mGfA * mAfA * mGfGmCfAmUfGmA * T * mU | OOXX | |
| WV- | TGCCACUGAGAAAGGCAUGAT | T * fG * mCfC * mAfC * mUfG * L010fA * mGfA | XXOXOXOXOXOXOXOOOO | 1593 |
| 9281 | U | * mAfA * mGfGmCfAmUfGmA * T * mU | OOXX | |
| WV- | TGCCACUGUGAAAGGCAUGAT | T * fG * mCfC * mAfC * mUfG * mUL010 * | XXOXOXOXOXOXOXOOOO | 1594 |
| 9282 | U | mGfA * mAfA * mGfGmCfAmUfGmA * T * mU | OOXX | |
| WV- | TGCCACUGUAAAAGGCAUGAT | T * fG * mCfC * mAfC * mUfG * mUfA * L010fA | XXOXOXOXOXOXOXOOOO | 1595 |
| 9283 | U | * mAfA * mGfGmCfAmUfGmA * T * mU | OOXX | |
| WV- | TGCCACUGUAGAAGGCAUGAT | T * fG * mCfC * mAfC * mUfG * mUfA * mGfA | XXOXOXOXOXOXOXOOOO | 1596 |
| 9284 | U | * mAL010 * mGfGmCfAmUfGmA * T * mU | OOXX | |
| WV- | TGUCCAGCUUUAUUGGGAGG | Mod001L0015MRdT * SfG * SmUmC * SmCmA | OSSOSOSOSSSOSSSSSSSSS | 1597 |
| 9392 | CTU | * SmGmC * SmU * SmU * SmUmA * SmU * | SS | |
| | | SfU * SmG * SmG * SmG * SmA * SmG * SmG | | |
| | | * SmC * ST * SmU | | |
| WV- | TGUCCAGCUUUAUUGGGAGG | L0015MRdT * SfG * SmUmC * SmCmA * | OSSOSOSOSSSOSSSSSSSSS | 1598 |
| 9393 | CTU | SmGmC * SmU * SmU * SmUmA * SmU * SfU | SS | |
| | | * SmG * SmG * SmG * SmA * SmG * SmG * | | |
| | | SmC * ST * SmU | | |
| WV- | TAGCUUCUUGUCCAGCUUUA | POT * SfA * SmGfC * SmU * SfU * SmC * SfU * | SSOSSSSSSSOSOSSSSSSSSS | 1599 |
| 9446 | UTU | SmU * SfG * SmUfC * SmCfA * SmG * SfC * | | |
| | | SmU * SfU * SmU * SfA * SmU * STGaNC6T * | | |
| | | SmU | | |
| WV- | TAGCUUCUUGUCCAGCUUUA | POT * SfA * SmGmC * SmU * SmU * SmC * | SSOSSSSSSSOSOSSSSSSSSS | 1600 |
| 9447 | UTU | SmU * SmU * SmG * SmUmC * SmCfA * SmG * | | |
| | | SmC * SmU * SmU * SmU * SmA * SmU * | | |
| | | STGaNC6T * SmU | | |
| WV- | TAGCUUCUUGUCCAGCUUUA | PST * SfA * SmGfC * SmU * SfU * SmC * SfU * | SSOSSSSSSSOSOSSSSSSSSS | 1601 |
| 9448 | UTU | SmU * SfG * SmUfC * SmCfA * SmG * SfC * | | |
| | | SmU * SfU * SmU * SfA * SmU * STGaNC6T * | | |
| | | SmU | | |
| WV- | TAGCUUCUUGUCCAGCUUUA | PST * SfA * SmGmC * SmU * SmU * SmC * | SSOSSSSSSSOSOSSSSSSSSS | 1602 |
| 9449 | UTU | SmU * SmU * SmG * SmUmC * SmCfA * SmG * | | |
| | | SmC * SmU * SmU * SmU * SmA * SmU * | | |
| | | STGaNC6T * SmU | | |
| WV- | TAGCUUCUUGUCCAGCUUUA | POT * SfA * SmGfC * SmU * SfU * SmC * SfU * | SSOSSSSSSSOSOSSSSSSSSS | 1603 |
| 9450 | UTU | SmU * SfG * SmUfC * SmCfA * SmG * SfC * | | |
| | | SmU * SfU * SmU * SfA * SmU * SAMC6T * | | |
| | | SmU | | |
| WV- | TAGCUUCUUGUCCAGCUUUA | POT * SfA * SmGmC * SmU * SmU * SmC * | SSOSSSSSSSOSOSSSSSSSSS | 1604 |
| 9451 | UTU | SmU * SmU * SmG * SmUmC * SmCfA * SmG * | | |
| | | SmC * SmU * SmU * SmU * SmA * SmU * | | |
| | | SAMC6T * SmU | | |
| WV- | TAGCUUCUUGUCCAGCUUUA | PST * SfA * SmGfC * SmU * SfU * SmC * SfU * | SSOSSSSSSSOSOSSSSSSSSS | 1605 |
| 9452 | UTU | SmU * SfG * SmUfC * SmCfA * SmG * SfC * | | |
| | | SmU * SfU * SmU * SfA * SmU * SAMC6T * | | |
| | | SmU | | |
| WV- | TAGCUUCUUGUCCAGCUUUA | PST * SfA * SmGmC * SmU * SmU * SmC * | SSOSSSSSSSOSOSSSSSSSSS | 1606 |
| 9453 | UTU | SmU * SmU * SmG * SmUmC * SmCfA * SmG * | | |
| | | SmC * SmU * SmU * SmU * SmA * SmU * | | |
| | | SAMC6T * SmU | | |
| WV- | TAGCUUCUUGUCCAGCUUUA | PO5MRdT * SfA * SmGmC * SmU * SmU * SmC | SSOSSSSSSSOSOSSSSSSSSS | 1607 |
| 9454 | UTU | * SmU * SmU * SmG * SmUmC * SmCfA * SmG | | |
| | | * SmC * SmU * SmU * SmU * SmA * SmU * | | |
| | | STGaNC6T * SmU | | |
| WV- | TAGCUUCUUGUCCAGCUUUA | PS5MRdT * SfA * SmGfC * SmU * SfU * SmC * | SSOSSSSSSSOSOSSSSSSSSS | 1608 |
| 9455 | UTU | SfU * SmU * SfG * SmUfC * SmCfA * SmG * SfC | | |
| | | * SmU * SfU * SmU * SfA * SmU * STGaNC6T * | | |
| | | SmU | | |
| WV- | TAGCUUCUUGUCCAGCUUUA | PS5MRdT * SfA * SmGmC * SmU * SmU * SmC | SSOSSSSSSSOSOSSSSSSSSS | 1609 |
| 9456 | UTU | * SmU * SmU * SmG * SmUmC * SmCfA * SmG | | |
| | | * SmC * SmU * SmU * SmU * SmA * SmU * | | |
| | | STGaNC6T * SmU | | |
| WV- | TAGCUUCUUGUCCAGCUUUA | PO5MRdT * SfA * SmGmC * SmU * SmU * SmC | SSOSSSSSSSOSOSSSSSSSSS | 1610 |
| 9457 | UTU | * SmU * SmU * SmG * SmUmC * SmCfA * SmG | | |
| | | * SmC * SmU * SmU * SmU * SmA * SmU * | | |
| | | SAMC6T * SmU | | |
| WV- | TAGCUUCUUGUCCAGCUUUA | PS5MRdT * SfA * SmGfC * SmU * SfU * SmC * | SSOSSSSSSSOSOSSSSSSSSS | 1611 |
| 9458 | UTU | SfU * SmU * SfG * SmUfC * SmCfA * SmG * SfC | | |
| | | * SmU * SfU * SmU * SfA * SmU * SAMC6T * | | |
| | | SmU | | |
| WV- | TAGCUUCUUGUCCAGCUUUA | PS5MRdT * SfA * SmGmC * SmU * SmU * SmC | SSOSSSSSSSOSOSSSSSSSSS | 1612 |
| 9459 | UTU | * SmU * SmU * SmG * SmUmC * SmCfA * SmG | | |
| | | * SmC * SmU * SmU * SmU * SmA * SmU * | | |
| | | SAMC6T * SmU | | |
| WV- | TGUCCAGCUUUAUUGGGAGG | 5tzdT * SfG * SmUfC * SmCfA * SmGfC * SmU | SSOSOSOSSSOSSSSSSSSSSS | 1613 |
| 9460 | CTU | * SfU * SmUfA * SmU * SfU * SmG * SfG * | | |
| | | SmG * SfA * SmG * SfG * SmC * SAMC6T * | | |
| | | SmU | | |
| WV- | TGUCCAGCUUUAUUGGGAGG | 5tzdT * SfG * SmUmC * SmCmA * SmGmC * | SSOSOSOSSSOSSSSSSSSSSS | 1614 |
| 9461 | CTU | SmU * SmU * SmUmA * SmU * SfU * SmG * | | |
| | | SmG * SmG * SmA * SmG * SmG * SmC * | | |
| | | SAMC6T * SmU | | |
| WV- | TAGCUUCUUGUCCAGCUUUA | 5tzdT * SfA * SmGfC * SmU * SfU * SmC * SfU | SSOSSSSSSSOSOSSSSSSSSS | 1615 |
| 9462 | UTU | * SmU * SfG * SmUfC * SmCfA * SmG * SfC * | | |
| | | SmU * SfU * SmU * SfA * SmU * SAMC6T * | | |
| | | SmU | | |
| WV- | TAGCUUCUUGUCCAGCUUUA | 5tzdT * SfA * SmGmC * SmU * SmU * SmC * | SSOSSSSSSSOSOSSSSSSSSS | 1616 |
| 9463 | UTU | SmU * SmU * SmG * SmUmC * SmCfA * SmG * | | |
| | | SmC * SmU * SmU * SmU * SmA * SmU * | | |
| | | SAMC6T * SmU | | |
| WV- | TGUCCAGCUUUAUUGGGAGG | T * fG * mUmC * mCmA * mGmC * mUmU * | XXOXOXOXOXOXOXXXXXX | 1617 |
| 9475 | CTU | mUmA * mUfU * mG * mG * mG * mA * mG * | XXX | |
| | | mG * fC * T * mU | | |
| WV- | TGUCCAGCUUUAUUGGGAGG | T * fG * mUmC * mCmA * mGmC * mUmU * | XXOXOXOXOXOXOXXXXXX | 1618 |
| 9476 | CTU | mUmA * mUfU * mG * mG * mG * mA * mG * | XXX | |
| | | fG * fC * T * mU | | |
| WV- | TGUCCAGCUUUAUUGGGAGG | T * fG * mUmC * mCmA * mGmC * mUmU * | XXOXOXOXOXOXOXXXXXX | 1619 |
| 9477 | CTU | mUmA * mUfU * mG * mG * mG * mA * fG * | XXX | |
| | | fG * fC * T * mU | | |
| WV- | TGUCCAGCUUUAUUGGGAGG | T * fG * mUmC * mCmA * mGmC * mUmU * | XXOXOXOXOXOXOXXXXXX | 1620 |
| 9478 | CTU | mUmA * mUfU * mG * mG * mG * fA * fG * fG | XXX | |
| | | * fC * T * mU | | |
| WV- | TGUCCAGCUUUAUUGGGAGG | T * fG * mUmC * mCmA * mGmC * mUmU * | XXOXOXOXOXOXOXXXXXX | 1621 |
| 9479 | CTU | mUmA * mUfU * mG * mG * fG * fA * fG * fG | XXX | |
| | | * fC * T * mU | | |
| WV- | TGUCCAGCUUUAUUGGGAGG | T * fG * fUmC * mCmA * mGmC * mUmU * | XXOXOXOXOXOXOXXXXXX | 1622 |
| 9480 | CTU | mUmA * mUfU * mG * mG * mG * mA * mG * | XXX | |
| | | fG * fC * T * mU | | |
| WV- | TGUCCAGCUUUAUUGGGAGG | T * fG * fUfC * mCmA * mGmC * mUmU * | XXOXOXOXOXOXOXXXXXX | 1623 |
| 9481 | CTU | mUmA * mUfU * mG * mG * mG * mA * fG * | XXX | |
| | | fG * fC * T * mU | | |
| WV- | TGUCCAGCUUUAUUGGGAGG | T * fG * fUfC * fCmA * mGmC * mUmU * | XXOXOXOXOXOXOXXXXXX | 1624 |
| 9482 | CTU | mUmA * mUfU * mG * mG * mG * fA * fG * fG | XXX | |
| | | * fC * T * mU | | |
| WV- | TGUCCAGCUUUAUUGGGAGG | T * fG * fUfC * fCfA * mGmC * mUmU * | XXOXOXOXOXOXOXXXXXX | 1625 |
| 9483 | CTU | mUmA * mUfU * mG * mG * fG * fA * fG * fG | XXX | |
| | | * fC * T * mU | | |
| WV- | AGCTUCTTGTCCAGCUUUAU | Mod001L001mA * SGeom5CeoTeomU * SC * | OSOOOSSSSRSSRSSSSSSS | 1626 |
| 9526 | | ST * ST * SG * RT * SC * SC * RA * SG * SC * | | |
| | | SfU * SfU * SfU * SfA * SfU | | |
| WV- | AGCTUCTTGTCCAGCUUUAU | Mod001L001mA * SGeom5CeoTeomU * SC * | OSOOOSSSSRSSSSSSSSSS | 1627 |
| 9527 | | ST * ST * SG * RT * SC * SC * SA * SG * SC * | | |
| | | SfU * SfU * SfU * SfA * SfU | | |
| WV- | AGCTUCTTGTCCAGCUUUAU | Mod001L001mA * SGeom5CeoTeomU * SC * | OSOOOSSSSSSSRSSSSSSS | 1628 |
| 9528 | | ST * ST * SG * ST * SC * SC * RA * SG * SC * | | |
| | | SfU * SfU * SfU * SfA * SfU | | |
| WV- | AGCTUCTTGTCCAGCUUUAU | Mod001L001mA * Geom5CeoTeomU * C * T * | OXOOOXXXXXXXXXXXXXXX | 1629 |
| 9529 | | T * G * T * C * C * A * G * C * fU * fU * fU * fA | | |
| | | * fU | | |
| WV- | AGCTUCTTGTCCAGCUUUAU | mA * SGeom5CeoTeomU * SC * ST * ST * SG * | SOOOSSSSRSSRSSSSSSS | 1630 |
| 9530 | | RT * SC * SC * RA * SG * SC * SfU * SfU * SfU * | | |
| | | SfA * SfU | | |
| WV- | AGCTUCTTGTCCAGCUUUAU | mA * SGeom5CeoTeomU * SC * ST * ST * SG * | SOOOSSSSRSSSSSSSSSS | 1631 |
| 9531 | | RT * SC * SC * SA * SG * SC * SfU * SfU * SfU * | | |
| | | SfA * SfU | | |
| WV- | AGCTUCTTGTCCAGCUUUAU | mA * SGeom5CeoTeomU * SC * ST * ST * SG * | SOOOSSSSSSSRSSSSSSS | 1632 |
| 9532 | | ST * SC * SC * RA * SG * SC * SfU * SfU * SfU * | | |
| | | SfA * SfU | | |
| WV- | AGCTUCTTGTCCAGCUUUAU | mA * Geom5CeoTeomU * C * T * T * G * T * C | XOOOXXXXXXXXXXXXXXX | 1633 |
| 9533 | | * C * A * G * C * fU * fU * fU * fA * fU | | |
| WV- | AGCTTCTTGTCCAGCTTTAT | Mod083L001Aeo * SGeom5CeoTeoTeo * RC * | OSOOORSSSRSSRSSROOOS | 1634 |
| 9542 | | ST * ST * SG * RT * SC * SC * RA * SG * SC * | | |
| | | RTeoTeoTeoAeo * STeo | | |
| WV- | AGCTTCTTGTCCAGCTTTAT | Mod079L001Aeo * SGeom5CeoTeoTeo * RC * | OSOOORSSSRSSRSSROOOS | 1635 |
| 9543 | | ST * ST * SG * RT * SC * SC * RA * SG * SC * | | |
| | | RTeoTeoTeoAeo * STeo | | |
| WV- | AGCTTCTTGTCCAGCTTTAT | Mod080L001Aeo * SGeom5CeoTeoTeo * RC * | OSOOORSSSRSSRSSROOOS | 1636 |
| 9544 | | ST * ST * SG * RT * SC * SC * RA * SG * SC * | | |
| | | RTeoTeoTeoAeo * STeo | | |
| WV- | AGCTTCTTGTCCAGCTTTAT | Mod081L001Aeo * SGeom5CeoTeoTeo * RC * | OSOOORSSSRSSRSSROOOS | 1637 |
| 9545 | | ST * ST * SG * RT * SC * SC * RA * SG * SC * | | |
| | | RTeoTeoTeoAeo * STeo | | |
| WV- | AGCTTCTTGTCCAGCTTTAT | Mod082L001Aeo * SGeom5CeoTeoTeo * RC * | OSOOORSSSRSSRSSROOOS | 1638 |
| 9546 | | ST * ST * SG * RT * SC * SC * RA * SG * SC * | | |
| | | RTeoTeoTeoAeo * STeo | | |
| WV- | TAGCUUCUUGUCCAGCUUUA | Mod001L001T * SfA * SmGfC * SmU * SfU * | OSSOSSSSSSSOSOSSSSSSSS | 1639 |
| 9557 | UTU | SmC * SfU * SmU * SfG * SmUfC * SmCfA * | S | |
| | | SmG * SfC * SmU * SfU * SmU * SfA * SmU * | | |
| | | ST * SmU | | |
| WV- | TAGCUUCUUGUCCAGCUUUA | Mod001L0015MRdT * SfA * SmGfC * SmU * | OSSOSSSSSSSOSOSSSSSSSS | 1640 |
| 9558 | UTU | SfU * SmC * SfU * SmU * SfG * SmUfC * SmCfA | S | |
| | | * SmG * SfC * SmU * SfU * SmU * SfA * SmU * | | |
| | | ST * SmU | | |
| WV- | TAGCUUCUUGUCCAGCUUUA | Mod001L001T * SfA * SmGmC * SmU * SmU * | OSSOSSSSSSSOSOSSSSSSSS | 1641 |
| 9559 | UTU | SmC * SmU * SmU * SmG * SmUmC * SmCfA * | S | |
| | | SmG * SmC * SmU * SmU * SmU * SmA * SmU | | |
| | | * ST * SmU | | |
| WV- | TAGCUUCUUGUCCAGCUUUA | Mod001L0015MRdT * SfA * SmGmC * SmU * | OSSOSSSSSSSOSOSSSSSSSS | 1642 |
| 9560 | UTU | SmU * SmC * SmU * SmU * SmG * SmUmC * | S | |
| | | SmCfA * SmG * SmC * SmU * SmU * SmU * | | |
| | | SmA * SmU * ST * SmU | | |
| WV- | TGUCCAGCUUUAUUGGGAGG | Mod001L001T * SfG * SmUmC * SmCmA * | OSSOSOSOSSSOSSSSSSSSS | 1643 |
| 9561 | CTU | SmGmC * SmU * SmU * SmUmA * SmU * SfU | SS | |
| | | * SmG * SmG * SmG * SmA * SmG * SmG * | | |
| | | SmC * ST * SmU | | |
| WV- | TAGCUUCUUGUCCAGCUUUA | L001T * SfA * SmGfC * SmU * SfU * SmC * SfU | OSSOSSSSSSSOSOSSSSSSSS | 1644 |
| 9581 | UTU | * SmU * SfG * SmUfC * SmCfA * SmG * SfC * | S | |
| | | SmU * SfU * SmU * SfA * SmU * ST * SmU | | |
| WV- | TAGCUUCUUGUCCAGCUUUA | L0015MRdT * SfA * SmGfC * SmU * SfU * SmC | OSSOSSSSSSSOSOSSSSSSSS | 1645 |
| 9582 | UTU | * SfU * SmU * SfG * SmUfC * SmCfA * SmG * | S | |
| | | SfC * SmU * SfU * SmU * SfA * SmU * ST * | | |
| | | SmU | | |
| WV- | TAGCUUCUUGUCCAGCUUUA | L001T * SfA * SmGmC * SmU * SmU * SmC * | OSSOSSSSSSSOSOSSSSSSSS | 1646 |
| 9583 | UTU | SmU * SmU * SmG * SmUmC * SmCfA * SmG * | S | |
| | | SmC * SmU * SmU * SmU * SmA * SmU * ST * | | |
| | | SmU | | |
| WV- | TAGCUUCUUGUCCAGCUUUA | L0015MRdT * SfA * SmGmC * SmU * SmU * | OSSOSSSSSSSOSOSSSSSSSS | 1647 |
| 9584 | UTU | SmC * SmU * SmU * SmG * SmUmC * SmCfA * | S | |
| | | SmG * SmC * SmU * SmU * SmU * SmA * SmU | | |
| | | * ST * SmU | | |
| WV- | TGUCCAGCUUUAUUGGGAGG | L001T * SfG * SmUmC * SmCmA * SmGmC * | OSSOSOSOSSSOSSSSSSSSS | 1648 |
| 9585 | CTU | SmU * SmU * SmUmA * SmU * SfU * SmG * | SS | |
| | | SmG * SmG * SmA * SmG * SmG * SmC * ST * | | |
| | | SmU | | |
| WV- | TGCCACUGUAGAAAGGCAUG | L001T * SfG * SmCfC * SmAfC * SmUfG * | OSSOSOSOSOSOSOSSSSSS | 1649 |
| 9586 | ATU | SmUfA * SmGfA * SmAfA * SmG * SfG * SmC * | SSS | |
| | | SfA * SmU * SfG * SmA * ST * SmU | | |
| WV- | TGCCACUGUAGAAAGGCAUG | L0015MRdT * SfG * SmCfC * SmAfC * SmUfG * | OSSOSOSOSOSOSOSSSSSS | 1650 |
| 9587 | ATU | SmUfA * SmGfA * SmAfA * SmG * SfG * SmC * | SSS | |
| | | SfA * SmU * SfG * SmA * ST * SmU | | |
| WV- | TGCCACUGUAGAAAGGCAUG | L001T * SfG * SmCmC * SmAmC * SmUmG * | OSSOSOSOSOSOSOSSSSSS | 1651 |
| 9588 | ATU | SmUmA * SmGmA * SmAfA * SmG * SmG * | SSS | |
| | | SmC * SmA * SmU * SmG * SmA * ST * SmU | | |
| WV- | TGCCACUGUAGAAAGGCAUG | L0015MRdT * SfG * SmCmC * SmAmC * | OSSOSOSOSOSOSOSSSSSS | 1652 |
| 9589 | ATU | SmUmG * SmUmA * SmGmA * SmAfA * SmG * | SSS | |
| | | SmG * SmC * SmA * SmU * SmG * SmA * ST * | | |
| | | SmU | | |
| WV- | AGCTUCTTGTCCAGCUUUAU | L001mA * SGeom5CeoTeomU * SC * ST * ST * | OSOOOSSSSRSSRSSSSSSS | 1653 |
| 9590 | | SG * RT * SC * SC * RA * SG * SC * SfU * SfU * | | |
| | | SfU * SfA * SfU | | |
| WV- | AGCTUCTTGTCCAGCUUUAU | L001mA * SGeom5CeoTeomU * SC * ST * ST * | OSOOOSSSSRSSSSSSSSSS | 1654 |
| 9591 | | SG * RT * SC * SC * SA * SG * SC * SfU * SfU * | | |
| | | SfU * SfA * SfU | | |
| WV- | AGCTUCTTGTCCAGCUUUAU | L001mA * SGeom5CeoTeomU * SC * ST * ST * | OSOOOSSSSSSSRSSSSSSS | 1655 |
| 9592 | | SG * ST * SC * SC * RA * SG * SC * SfU * SfU * | | |
| | | SfU * SfA * SfU | | |
| WV- | AGCTUCTTGTCCAGCUUUAU | L001mA * Geom5CeoTeomU * C * T * T * G * | OXOOOXXXXXXXXXXXXXXX | 1656 |
| 9593 | | T * C * C * A * G * C * fU * fU * fU * fA * fU | | |
| WV- | TCACUGAGAAUACUGUCCCUU | T * fC * mAfC * mUfG * mAfG * mAfA * mUfA | XXOXOXOXOXOXOXXXXXX | 1657 |
| 9705 | TU | * mCfU * mG * fU * mC * fC * mC * fU * mU * | XXX | |
| | | T * mU | | |
| WV- | TCACUGAGAAUACUGUCCCUU | T * fC * mAmC * mUmG * mAmG * mAmA * | XXOXOXOXOXOXOXXXXXX | 1658 |
| 9706 | TU | mUmA * mCfU * mG * mU * mC * mC * mC * | XXX | |
| | | mU * mU * T * mU | | |
| WV- | TCACUGAGAAUACUGUCCCUU | T * SfC * SmAfC * SmUfG * SmAfG * SmAfA * | SSOSOSOSOSOSOSSSSSSSS | 1659 |
| 9707 | TU | SmUfA * SmCfU * SmG * SfU * SmC * SfC * | S | |
| | | SmC * SfU * SmU * ST * SmU | | |
| WV- | TCACUGAGAAUACUGUCCCUU | T * SfC * SmAmC * SmUmG * SmAmG * | SSOSOSOSOSOSOSSSSSSSS | 1660 |
| 9708 | TU | SmAmA * SmUmA * SmCfU * SmG * SmU * | S | |
| | | SmC * SmC * SmC * SmU * SmU * ST * SmU | | |
| WV- | TCACUGAGAAUACUGUCCCUU | T * fC * mAfC * mU * fG * mAfG * mAfA * mU | XXOXXXOXOXXXXXXXXXXX | 1661 |
| 9716 | TU | * fA * mC * fU * mG * fU * mC * fC * mC * fU * | XX | |
| | | mU * T * mU | | |
| WV- | TCACUGAGAAUACUGUCCCUU | T * fC * mA * fC * mU * fG * mAfG * mAfA * | XXXXXXOXOXXXXXXXXXXX | 1662 |
| 9717 | TU | mU * fA * mC * fU * mG * fU * mC * fC * mC * | XX | |
| | | fU * mU * T * mU | | |
| WV- | TCACUGAGAAUACUGUCCCUU | T * fC * mAfC * mU * fG * mA * fG * mAfA * | XXOXXXXXOXXXXXXXXXXX | 1663 |
| 9718 | TU | mU * fA * mC * fU * mG * fU * mC * fC * mC * | XX | |
| | | fU * mU * T * mU | | |
| WV- | TCACUGAGAAUACUGUCCCUU | T * fC * mAfC * mU * fG * mAfG * mA * fA * | XXOXXXOXXXXXXXXXXXXX | 1664 |
| 9719 | TU | mU * fA * mC * fU * mG * fU * mC * fC * mC * | XX | |
| | | fU * mU * T * mU | | |
| WV- | TCACUGAGAAUACUGUCCCUU | T * fC * mA * fC * mU * fG * mA * fG * mAfA * | XXXXXXXXOXXXXXXXXXXX | 1665 |
| 9720 | TU | mU * fA * mC * fU * mG * fU * mC * fC * mC * | XX | |
| | | fU * mU * T * mU | | |
| WV- | TCACUGAGAAUACUGUCCCUU | T * fC * mA * fC * mU * fG * mAfG * mA * fA * | XXXXXXOXXXXXXXXXXXXX | 1666 |
| 9721 | TU | mU * fA * mC * fU * mG * fU * mC * fC * mC * | XX | |
| | | fU * mU * T * mU | | |
| WV- | TCACUGAGAAUACUGUCCCUU | T * fC * mAfC * mU * fG * mA * fG * mA * fA * | XXOXXXXXXXXXXXXXXXXX | 1667 |
| 9722 | TU | mU * fA * mC * fU * mG * fU * mC * fC * mC * | XX | |
| | | fU * mU * T * mU | | |
| WV- | TCACUGAGAAUACUGUCCCUU | T * fC * mA * fC * mU * fG * mA * fG * mA * fA | XXXXXXXXXXXXXXXXXXXXX | 1668 |
| 9723 | TU | * mU * fA * mC * fU * mG * fU * mC * fC * mC | X | |
| | | * fU * mU * T * mU | | |
| WV- | TCACUGAGAAUACUGUCCCUU | T * fC * mAmC * mU * mG * mAmG * mAmA * | XXOXXXOXOXXXXXXXXXXX | 1669 |
| 9724 | TU | mU * mA * mC * fU * mG * mU * mC * mC * | XX | |
| | | mC * mU * mU * T * mU | | |
| WV- | TCACUGAGAAUACUGUCCCUU | T * fC * mA * mC * mU * mG * mAmG * | XXXXXXOXOXXXXXXXXXXX | 1670 |
| 9725 | TU | mAmA * mU * mA * mC * fU * mG * mU * mC | XX | |
| | | * mC * mC * mU * mU * T * mU | | |
| WV- | TCACUGAGAAUACUGUCCCUU | T * fC * mAmC * mU * mG * mA * mG * | XXOXXXXXOXXXXXXXXXXX | 1671 |
| 9726 | TU | mAmA * mU * mA * mC * fU * mG * mU * mC | XX | |
| | | * mC * mC * mU * mU * T * mU | | |
| WV- | TCACUGAGAAUACUGUCCCUU | T * fC * mAmC * mU * mG * mAmG * mA * | XXOXXXOXXXXXXXXXXXXX | 1672 |
| 9727 | TU | mA * mU * mA * mC * fU * mG * mU * mC * | XX | |
| | | mC * mC * mU * mU * T * mU | | |
| WV- | TCACUGAGAAUACUGUCCCUU | T * fC * mA * mC * mU * mG * mA * mG * | XXXXXXXXOXXXXXXXXXXX | 1673 |
| 9728 | TU | mAmA * mU * mA * mC * fU * mG * mU * mC | XX | |
| | | mC * mC * mU * mU * T * mU | | |
| WV- | TCACUGAGAAUACUGUCCCUU | T * fC * mA * mC * mU * mG * mAmG * mA * | XXXXXXOXXXXXXXXXXXXX | 1674 |
| 9729 | TU | mA * mU * mA * mC * fU * mG * mU * mC * | XX | |
| | | mC * mC * mU * mU * T * mU | | |
| WV- | TCACUGAGAAUACUGUCCCUU | T * fC * mAmC * mU * mG * mA * mG * mA * | XXOXXXXXXXXXXXXXXXXX | 1675 |
| 9730 | TU | mA * mU * mA * mC * fU * mG * mU * mC * | XX | |
| | | mC * mC * mU * mU * T * mU | | |
| WV- | TCACUGAGAAUACUGUCCCUU | T * fC * mA * mC * mU * mG * mA * mG * mA | XXXXXXXXXXXXXXXXXXXXX | 1676 |
| 9731 | TU | * mA * mU * mA * mC * fU * mG * mU * mC * | X | |
| | | mC * mC * mU * mU * T * mU | | |
| WV- | TGUCCAGCUUUAUUGGGAGG | VPT * SfG * SmUfC * SmCfA * SmGfC * SmU * | SSOSOSOSSSOSSSSSSSSSSS | 1677 |
| 9863 | CTU | SmU * SmUmA * SmU * SfU * SmG * SmG * | | |
| | | SmG * SmA * SmG * SmG * SmC * STGaNC6T * | | |
| | | SmU | | |
| WV- | TGUCCAGCUUUAUUGGGAGG | VPT * SfG * SmUmC * SmCmA * SmGmC * | SSOSOSOSSSOSSSSSSSSSSS | 1678 |
| 9864 | CTU | SmU * SmU * SmUmA * SmU * SfU * SmG * | | |
| | | SmG * SmG * SmA * SmG * SmG * SmC * | | |
| | | STGaNC6T * SmU | | |
| WV- | TAGCUUCUUGUCCAGCUUUA | VPT * SfA * SmGmC * SmU * SmU * SmC * | SSOSSSSSSSOSOSSSSSSSSS | 1679 |
| 9865 | UTU | SmU * SmU * SmG * SmUmC * SmCfA * SmG * | | |
| | | SmC * SmU * SmU * SmU * SmA * SmU * | | |
| | | STGaNC6T * SmU | | |
| WV- | AGCTTCTTGTCCAGCUUUAU | Mod001L001mA * Geom5CeoTeoTeo * C * T * | OXOOOXXXXXXXXXXXXXXX | 1680 |
| 9871 | | T * G * T * C * C * A * G * C * mU * mU * mU * | | |
| | | mA * mU | | |
| WV- | AGCTTCTTGTCCAGCUUUAU | Mod001L001mA * SGeom5CeoTeoTeo * RC * | OSOOORSSSRSSRSSSSSSS | 1681 |
| 9872 | | ST * ST * SG * RT * SC * SC * RA * SG * SC * | | |
| | | SmU * SmU * SmU * SmA * SmU | | |
| WV- | AGCTTCTTGTCCAGCTUUAU | Mod001L001mA * Geom5CeoTeoTeo * C * T * | OXOOOXXXXXXXXXXXXXXX | 1682 |
| 9873 | | T * G * T * C * C * A * G * C * Teo * mU * mU * | | |
| | | mA * mU | | |
| WV- | AGCTTCTTGTCCAGCTUUAU | Mod001L001mA * SGeom5CeoTeoTeo * RC * | OSOOORSSSRSSRSSRSSSS | 1683 |
| 9874 | | ST * ST * SG * RT * SC * SC * RA * SG * SC * | | |
| | | RTeo * SmU * SmU * SmA * SmU | | |
| WV- | TGUCCAGCUUUAUUGGGAGG | VPT * SfG * SmUfC * SmCfA * SmGfC * SmU * | SSOSOSOSSSOSSSSSSSSSSS | 1684 |
| 9880 | CTU | SmU * SmUmA * SmU * SfU * SmG * SmG * | | |
| | | SmG * SmA * SmG * SmG * SmC * SAMC6T * | | |
| | | SmU | | |
| WV- | TGUCCAGCUUUAUUGGGAGG | VPT * SfG * SmUmC * SmCmA * SmGmC * | SSOSOSOSSSOSSSSSSSSSSS | 1685 |
| 9881 | CTU | SmU * SmU * SmUmA * SmU * SfU * SmG * | | |
| | | SmG * SmG * SmA * SmG * SmG * SmC * | | |
| | | SAMC6T * SmU | | |
| WV- | TAGCUUCUUGUCCAGCUUUA | VPT * SfA * SmGmC * SmU * SmU * SmC * | SSOSSSSSSSOSOSSSSSSSSS | 1686 |
| 9882 | UTU | SmU * SmU * SmG * SmUmC * SmCfA * SmG * | | |
| | | SmC * SmU * SmU * SmU * SmA * SmU * | | |
| | | SAMC6T * SmU | | |
| WV- | AGCTTCTTGTCCAGCUUUAU | mA * Geom5CeoTeoTeo * C * T * T * G * T * C | XOOOXXXXXXXXXXXXXXX | 1687 |
| 9885 | | * C * A * G * C * mU * mU * mU * mA * mU | | |
| WV- | AGCTTCTTGTCCAGCUUUAU | mA * SGeom5CeoTeoTeo * RC * ST * ST * SG * | SOOORSSSRSSRSSSSSSS | 1688 |
| 9886 | | RT * SC * SC * RA * SG * SC * SmU * SmU * | | |
| | | SmU * SmA * SmU | | |
| WV- | AGCTTCTTGTCCAGCTUUAU | mA * Geom5CeoTeoTeo * C * T * T * G * T * C | XOOOXXXXXXXXXXXXXXX | 1689 |
| 9887 | | * C * A * G * C * Teo * mU * mU * mA * mU | | |
| WV- | AGCTTCTTGTCCAGCTUUAU | mA * SGeom5CeoTeoTeo * RC * ST * ST * SG * | SOOORSSSRSSRSSRSSSS | 1690 |
| 9888 | | RT * SC * SC * RA * SG * SC * RTeo * SmU * | | |
| | | SmU * SmA * SmU | | |
| WV- | AGCTTCTTGTCCAGCUUUAU | L001mA * Geom5CeoTeoTeo * C * T * T * G * | OXOOOXXXXXXXXXXXXXX | 1691 |
| 10243 | | T * C * C * A * G * C * mU * mU * mU * mA * | X | |
| | | mU | | |
| WV- | AGCTTCTTGTCCAGCUUUAU | L001mA * SGeom5CeoTeoTeo * RC * ST * ST * | OSOOORSSSRSSRSSSSSSS | 1692 |
| 10244 | | SG * RT * SC * SC * RA * SG * SC * SmU * SmU | | |
| | | * SmU * SmA * SmU | | |
| WV- | AGCTTCTTGTCCAGCTUUAU | L001mA * Geom5CeoTeoTeo * C * T * T * G * | OXOOOXXXXXXXXXXXXXX | 1693 |
| 10245 | | T * C * C * A * G * C * Teo * mU * mU * mA * | X | |
| | | mU | | |
| WV- | AGCTTCTTGTCCAGCTUUAU | L001mA * SGeom5CeoTeoTeo * RC * ST * ST * | OSOOORSSSRSSRSSRSSSS | 1694 |
| 10246 | | SG * RT * SC * SC * RA * SG * SC * RTeo * SmU | | |
| | | * SmU * SmA * SmU | | |
| WV- | TGUCCAGCUUUAUUGGGAGG | 5tzdT * SfG * SmUfC * SmCfA * SmGfC * SmU | SSOSOSOSSSOSSSSSSSSSS | 1695 |
| 10308 | CTU | * SfU * SmUfA * SmU * SfU * SmG * SfG * | S | |
| | | SmG * SfA * SmG * SfG * SmC * STGaNC6T * | | |
| | | SmU | | |
| WV- | TGUCCAGCUUUAUUGGGAGG | 5tzdT * SfG * SmUmC * SmCmA * SmGmC * | SSOSOSOSSSOSSSSSSSSSS | 1696 |
| 10309 | CTU | SmU * SmU * SmUmA * SmU * SfU * SmG * | S | |
| | | SmG * SmG * SmA * SmG * SmG * SmC * | | |
| | | STGaNC6T * SmU | | |
| WV- | TAGCUUCUUGUCCAGCUUUA | 5tzdT * SfA * SmGfC * SmU * SfU * SmC * SfU | SSOSSSSSSSOSOSSSSSSSSS | 1697 |
| 10310 | UTU | * SmU * SfG * SmUfC * SmCfA * SmG * SfC * | | |
| | | SmU * SfU * SmU * SfA * SmU * STGaNC6T * | | |
| | | SmU | | |
| WV- | TAGCUUCUUGUCCAGCUUUA | 5tzdT * SfA * SmGmC * SmU * SmU * SmC * | SSOSSSSSSSOSOSSSSSSSSS | 1698 |
| 10311 | UTU | SmU * SmU * SmG * SmUmC * SmCfA * SmG * | | |
| | | SmC * SmU * SmU * SmU * SmA * SmU * | | |
| | | STGaNC6T * SmU | | |
| WV- | TCCUUCCCUGAAGGUUCCUC | VPT * fC * mCfU * mUfC * mCfC * mUfG * | XXOXOXOXOXOXOXXXXXX | 1699 |
| 10312 | CTU | mAfA * mGfG * mU * fU * mC * fC * mU * fC * | XXX | |
| | | mC * T * mU | | |
| WV- | TCCUUCCCUGAAGGUUCCUC | VPT * fC * mCmU * mUmC * mCmC * mUmG * | XXOXOXOXOXOXOXXXXXX | 1700 |
| 10313 | CTU | mAmA * mGfG * mU * mU * mC * mC * mU * | XXX | |
| | | mC * mC * T * mU | | |
| WV- | TCCUUCCCUGAAGGUUCCUC | PO5MRdT * fC * mCfU * mUfC * mCfC * mUfG | XXOXOXOXOXOXOXXXXXX | 1701 |
| 10314 | CTU | * mAfA * mGfG * mU * fU * mC * fC * mU * fC | XXX | |
| | | * mC * T * mU | | |
| WV- | TCCUUCCCUGAAGGUUCCUC | PO5MRdT * fC * mCmU * mUmC * mCmC * | XXOXOXOXOXOXOXXXXXX | 1702 |
| 10315 | CTU | mUmG * mAmA * mGfG * mU * mU * mC * | XXX | |
| | | mC * mU * mC * mC * T * mU | | |
| WV- | TCCUUCCCUGAAGGUUCCUC | VPT * SfC * SmCfU * SmUfC * SmCfC * SmUfG | SSOSOSOSOSOSOSSSSSSSS | 1703 |
| 10316 | CTU | * SmAfA * SmGfG * SmU * SfU * SmC * SfC * | S | |
| | | SmU * SfC * SmC * ST * SmU | | |
| WV- | TCCUUCCCUGAAGGUUCCUC | VPT * SfC * SmCmU * SmUmC * SmCmC * | SSOSOSOSOSOSOSSSSSSSS | 1704 |
| 10317 | CTU | SmUmG * SmAmA * SmGfG * SmU * SmU * | S | |
| | | SmC * SmC * SmU * SmC * SmC * ST * SmU | | |
| WV- | TCCUUCCCUGAAGGUUCCUC | PO5MRdT * SfC * SmCfU * SmUfC * SmCfC * | SSOSOSOSOSOSOSSSSSSSS | 1705 |
| 10318 | CTU | SmUfG * SmAfA * SmGfG * SmU * SfU * SmC * | S | |
| | | SfC * SmU * SfC * SmC * ST * SmU | | |
| WV- | TCCUUCCCUGAAGGUUCCUC | PO5MRdT * SfC * SmCmU * SmUmC * SmCmC | SSOSOSOSOSOSOSSSSSSSS | 1706 |
| 10319 | CTU | * SmUmG * SmAmA * SmGfG * SmU * SmU * | S | |
| | | SmC * SmC * SmU * SmC * SmC * ST * SmU | | |
| WV- | TCACUGAGAAUACUGUCCCU | T * SfC * SmAmC * SmU * SmG * SmAmG * | SSOSSSOSOSSSSSSSSSSSSS | 1707 |
| 10471 | UTU | SmAmA * SmU * SmA * SmC * SfU * SmG * | | |
| | | SmU * SmC * SmC * SmC * SmU * SmU * ST * | | |
| | | SmU | | |
| WV- | TCACUGAGAAUACUGUCCCU | 5MRdT * SfC * SmAmC * SmU * SmG * | SSOSSSOSOSSSSSSSSSSSSS | 1708 |
| 10472 | UTU | SmAmG * SmAmA * SmU * SmA * SmC * SfU * | | |
| | | SmG * SmU * SmC * SmC * SmC * SmU * SmU | | |
| | | * ST * SmU | | |
| WV- | TCACUGAGAAUACUGUCCCU | PO5MRdT * SfC * SmAmC * SmU * SmG * | SSOSSSOSOSSSSSSSSSSSSS | 1709 |
| 10473 | UTU | SmAmG * SmAmA * SmU * SmA * SmC * SfU * | | |
| | | SmG * SmU * SmC * SmC * SmC * SmU * SmU | | |
| | | * ST * SmU | | |
| WV- | TCACUGAGAAUACUGUCCCU | VPT * SfC * SmAmC * SmU * SmG * SmAmG * | SSOSSSOSOSSSSSSSSSSSSS | 1710 |
| 10474 | UTU | SmAmA * SmU * SmA * SmC * SfU * SmG * | | |
| | | SmU * SmC * SmC * SmC * SmU * SmU * ST * | | |
| | | SmU | | |
| WV- | TCACUGAGAAUACUGUCCCU | T * SfC * SmAmC * SmU * SmG * SmAmG * | SSOSSSOSOSSSSSSSSSSSSS | 1711 |
| 10475 | UTU | SmAmA * SmU * SmA * SmC * SfU * SmG * | | |
| | | SmU * SmC * SmC * SmC * SmU * SmU * | | |
| | | STGaNC6T * SmU | | |
| WV- | TCACUGAGAAUACUGUCCCU | 5MRdT * SfC * SmAmC * SmU * SmG * | SSOSSSOSOSSSSSSSSSSSSS | 1712 |
| 10476 | UTU | SmAmG * SmAmA * SmU * SmA * SmC * SfU * | | |
| | | SmG * SmU * SmC * SmC * SmC * SmU * SmU | | |
| | | * STGaNC6T * SmU | | |
| WV- | TCACUGAGAAUACUGUCCCU | PO5MRdT * SfC * SmAmC * SmU * SmG * | SSOSSSOSOSSSSSSSSSSSSS | 1713 |
| 10477 | UTU | SmAmG * SmAmA * SmU * SmA * SmC * SfU * | | |
| | | SmG * SmU * SmC * SmC * SmC * SmU * SmU | | |
| | | * STGaNC6T * SmU | | |
| WV- | TCACUGAGAAUACUGUCCCU | VPT * SfC * SmAmC * SmU * SmG * SmAmG * | SSOSSSOSOSSSSSSSSSSSSS | 1714 |
| 10478 | UTU | SmAmA * SmU * SmA * SmC * SfU * SmG * | | |
| | | SmU * SmC * SmC * SmC * SmU * SmU * | | |
| | | STGaNC6T * SmU | | |
| WV- | TCACUGAGAAUACUGUCCCU | T * SfC * SmAmC * SmU * SmG * SmAmG * | SSOSSSOSOSSSSSSSSSSSSS | 1715 |
| 10479 | UTU | SmAmA * SmU * SmA * SmC * SfU * SmG * | | |
| | | SmU * SmC * SmC * SmC * SmU * SmU * | | |
| | | SAMC6T * SmU | | |
| WV- | TCACUGAGAAUACUGUCCCU | 5MRdT * SfC * SmAmC * SmU * SmG * | SSOSSSOSOSSSSSSSSSSSSS | 1716 |
| 10480 | UTU | SmAmG * SmAmA * SmU * SmA * SmC * SfU * | | |
| | | SmG * SmU * SmC * SmC * SmC * SmU * SmU | | |
| | | * SAMC6T * SmU | | |
| WV- | TCACUGAGAAUACUGUCCCU | PO5MRdT * SfC * SmAmC * SmU * SmG * | SSOSSSOSOSSSSSSSSSSSSS | 1717 |
| 10481 | UTU | SmAmG * SmAmA * SmU * SmA * SmC * SfU * | | |
| | | SmG * SmU * SmC * SmC * SmC * SmU * SmU | | |
| | | * SAMC6T * SmU | | |
| WV- | TCACUGAGAAUACUGUCCCU | VPT * SfC * SmAmC * SmU * SmG * SmAmG * | SSOSSSOSOSSSSSSSSSSSSS | 1718 |
| 10482 | UTU | SmAmA * SmU * SmA * SmC * SfU * SmG * | | |
| | | SmU * SmC * SmC * SmC * SmU * SmU * | | |
| | | SAMC6T * SmU | | |
| WV- | TCACUGAGAAUACUGUCCCU | 5tzdT * SfC * SmAmC * SmU * SmG * SmAmG | SSOSSSOSOSSSSSSSSSSSSS | 1719 |
| 10645 | UTU | * SmAmA * SmU * SmA * SmC * SfU * SmG * | | |
| | | SmU * SmC * SmC * SmC * SmU * SmU * ST * | | |
| | | SmU | | |
| WV- | TCACUGAGAAUACUGUCCCU | 5MRdT * SfC * SmAmC * SmUmG * SmAmG * | SSOSOSOSOSOSOSSSSSSSS | 1720 |
| 10646 | UTU | SmAmA * SmUmA * SmCfU * SmG * SmU * | S | |
| | | SmC * SmC * SmC * SmU * SmU * ST * SmU | | |
| WV- | TCACUGAGAAUACUGUCCCU | PO5MRdT * SfC * SmAmC * SmUmG * SmAmG | SSOSOSOSOSOSOSSSSSSSS | 1721 |
| 10647 | UTU | * SmAmA * SmUmA * SmCfU * SmG * SmU * | S | |
| | | SmC * SmC * SmC * SmU * SmU * ST * SmU | | |
| WV- | TCACUGAGAAUACUGUCCCU | VPT * SfC * SmAmC * SmUmG * SmAmG * | SSOSOSOSOSOSOSSSSSSSS | 1722 |
| 10648 | UTU | SmAmA * SmUmA * SmCfU * SmG * SmU * | S | |
| | | SmC * SmC * SmC * SmU * SmU * ST * SmU | | |
| WV- | TCACUGAGAAUACUGUCCCU | 5tzdT * SfC * SmAmC * SmUmG * SmAmG * | SSOSOSOSOSOSOSSSSSSSS | 1723 |
| 10649 | UTU | SmAmA * SmUmA * SmCfU * SmG * SmU * | S | |
| | | SmC * SmC * SmC * SmU * SmU * ST * SmU | | |
| WV- | TCACUGAGAAUACUGUCCCU | 5tzdT * SfC * SmAmC * SmU * SmG * SmAmG | SSOSSSOSOSSSSSSSSSSSSS | 1724 |
| 10650 | UTU | * SmAmA * SmU * SmA * SmC * SfU * SmG * | | |
| | | SmU * SmC * SmC * SmC * SmU * SmU * | | |
| | | STGaNC6T * SmU | | |
| WV- | TCACUGAGAAUACUGUCCCU | T * SfC * SmAmC * SmUmG * SmAmG * | SSOSOSOSOSOSOSSSSSSSS | 1725 |
| 10651 | UTU | SmAmA * SmUmA * SmCfU * SmG * SmU * | S | |
| | | SmC * SmC * SmC * SmU * SmU * STGaNC6T * | | |
| | | SmU | | |
| WV- | TCACUGAGAAUACUGUCCCU | 5MRdT * SfC * SmAmC * SmUmG * SmAmG * | SSOSOSOSOSOSOSSSSSSSS | 1726 |
| 10652 | UTU | SmAmA * SmUmA * SmCfU * SmG * SmU * | S | |
| | | SmC * SmC * SmC * SmU * SmU * STGaNC6T * | | |
| | | SmU | | |
| WV- | TCACUGAGAAUACUGUCCCU | PO5MRdT * SfC * SmAmC * SmUmG * SmAmG | SSOSOSOSOSOSOSSSSSSSS | 1727 |
| 10653 | UTU | * SmAmA * SmUmA * SmCfU * SmG * SmU * | S | |
| | | SmC * SmC * SmC * SmU * SmU * STGaNC6T * | | |
| | | SmU | | |
| WV- | TCACUGAGAAUACUGUCCCU | VPT * SfC * SmAmC * SmUmG * SmAmG * | SSOSOSOSOSOSOSSSSSSSS | 1728 |
| 10654 | UTU | SmAmA * SmUmA * SmCfU * SmG * SmU * | S | |
| | | SmC * SmC * SmC * SmU * SmU * STGaNC6T * | | |
| | | SmU | | |
| WV- | TCACUGAGAAUACUGUCCCU | 5tzdT * SfC * SmAmC * SmUmG * SmAmG * | SSOSOSOSOSOSOSSSSSSSS | 1729 |
| 10655 | UTU | SmAmA * SmUmA * SmCfU * SmG * SmU * | S | |
| | | SmC * SmC * SmC * SmU * SmU * STGaNC6T * | | |
| | | SmU | | |
| WV- | TCACUGAGAAUACUGUCCCU | 5tzdT * SfC * SmAmC * SmU * SmG * SmAmG | SSOSSSOSOSSSSSSSSSSSSS | 1730 |
| 10656 | UTU | * SmAmA * SmU * SmA * SmC * SfU * SmG * | | |
| | | SmU * SmC * SmC * SmC * SmU * SmU * | | |
| | | SAMC6T * SmU | | |
| WV- | TCACUGAGAAUACUGUCCCU | T * SfC * SmAmC * SmUmG * SmAmG * | SSOSOSOSOSOSOSSSSSSSS | 1731 |
| 10657 | UTU | SmAmA * SmUmA * SmCfU * SmG * SmU * | S | |
| | | SmC * SmC * SmC * SmU * SmU * SAMC6T * | | |
| | | SmU | | |
| WV- | TCACUGAGAAUACUGUCCCU | 5MRdT * SfC * SmAmC * SmUmG * SmAmG * | SSOSOSOSOSOSOSSSSSSSS | 1732 |
| 10658 | UTU | SmAmA * SmUmA * SmCfU * SmG * SmU * | S | |
| | | SmC * SmC * SmC * SmU * SmU * SAMC6T * | | |
| | | SmU | | |
| WV- | TCACUGAGAAUACUGUCCCU | PO5MRdT * SfC * SmAmC * SmUmG * SmAmG | SSOSOSOSOSOSOSSSSSSSS | 1733 |
| 10659 | UTU | * SmAmA * SmUmA * SmCfU * SmG * SmU * | S | |
| | | SmC * SmC * SmC * SmU * SmU * SAMC6T * | | |
| | | SmU | | |
| WV- | TCACUGAGAAUACUGUCCCU | VPT * SfC * SmAmC * SmUmG * SmAmG * | SSOSOSOSOSOSOSSSSSSSS | 1734 |
| 10660 | UTU | SmAmA * SmUmA * SmCfU * SmG * SmU * | S | |
| | | SmC * SmC * SmC * SmU * SmU * SAMC6T * | | |
| | | SmU | | |
| WV- | TCACUGAGAAUACUGUCCCU | 5tzdT * SfC * SmAmC * SmUmG * SmAmG * | SSOSOSOSOSOSOSSSSSSSS | 1735 |
| 10661 | UTU | SmAmA * SmUmA * SmCfU * SmG * SmU * | S | |
| | | SmC * SmC * SmC * SmU * SmU * SAMC6T * | | |
| | | SmU | | |
| WV- | TAGCUUCUUGUCCAGCUUUA | T * SfA * SmGmC * SmUmU * SmCmU * | SSOSOSOSOSOSOSSSSSSSS | 1736 |
| 10673 | UTU | SmUmG * SmUmC * SmCfA * SmG * SmC * | S | |
| | | SmU * SmU * SmU * SmA * SmU * STGaNC6T | | |
| | | * SmU | | |
| WV- | TAGCUUCUUGUCCAGCUUUA | 5MRdT * SfA * SmGmC * SmUmU * SmCmU * | SSOSOSOSOSOSOSSSSSSSS | 1737 |
| 10674 | UTU | SmUmG * SmUmC * SmCfA * SmG * SmC * | S | |
| | | SmU * SmU * SmU * SmA * SmU * STGaNC6T | | |
| | | * SmU | | |
| WV- | TAGCUUCUUGUCCAGCUUUA | PO5MRdT * SfA * SmGmC * SmUmU * SmCmU | SSOSOSOSOSOSOSSSSSSSS | 1738 |
| 10675 | UTU | * SmUmG * SmUmC * SmCfA * SmG * SmC * | S | |
| | | SmU * SmU * SmU * SmA * SmU * STGaNC6T | | |
| | | * SmU | | |
| WV- | TAGCUUCUUGUCCAGCUUUA | VPT * SfA * SmGmC * SmUmU * SmCmU * | SSOSOSOSOSOSOSSSSSSSS | 1739 |
| 10676 | UTU | SmUmG * SmUmC * SmCfA * SmG * SmC * | S | |
| | | SmU * SmU * SmU * SmA * SmU * STGaNC6T | | |
| | | * SmU | | |
| WV- | TAGCUUCUUGUCCAGCUUUA | 5tzdT * SfA * SmGmC * SmUmU * SmCmU * | SSOSOSOSOSOSOSSSSSSSS | 1740 |
| 10677 | UTU | SmUmG * SmUmC * SmCfA * SmG * SmC * | S | |
| | | SmU * SmU * SmU * SmA * SmU * STGaNC6T | | |
| | | * SmU | | |
| WV- | TAGCUUCUUGUCCAGCUUUA | T * SfA * SmGmC * SmUmU * SmCmU * | SSOSOSOSOSOSOSSSSSSSS | 1741 |
| 10678 | UTU | SmUmG * SmUmC * SmCfA * SmG * SmC * | S | |
| | | SmU * SmU * SmU * SmA * SmU * SAMC6T * | | |
| | | SmU | | |
| WV- | TAGCUUCUUGUCCAGCUUUA | 5MRdT * SfA * SmGmC * SmUmU * SmCmU * | SSOSOSOSOSOSOSSSSSSSS | 1742 |
| 10679 | UTU | SmUmG * SmUmC * SmCfA * SmG * SmC * | S | |
| | | SmU * SmU * SmU * SmA * SmU * SAMC6T * | | |
| | | SmU | | |
| WV- | TAGCUUCUUGUCCAGCUUUA | PO5MRdT * SfA * SmGmC * SmUmU * SmCmU | SSOSOSOSOSOSOSSSSSSSS | 1743 |
| 10680 | UTU | * SmUmG * SmUmC * SmCfA * SmG * SmC * | S | |
| | | SmU * SmU * SmU * SmA * SmU * SAMC6T * | | |
| | | SmU | | |
| WV- | TAGCUUCUUGUCCAGCUUUA | VPT * SfA * SmGmC * SmUmU * SmCmU * | SSOSOSOSOSOSOSSSSSSSS | 1744 |
| 10681 | UTU | SmUmG * SmUmC * SmCfA * SmG * SmC * | S | |
| | | SmU * SmU * SmU * SmA * SmU * SAMC6T * | | |
| | | SmU | | |
| WV- | TAGCUUCUUGUCCAGCUUUA | 5tzdT * SfA * SmGmC * SmUmU * SmCmU * | SSOSOSOSOSOSOSSSSSSSS | 1745 |
| 10682 | UTU | SmUmG * SmUmC * SmCfA * SmG * SmC * | S | |
| | | SmU * SmU * SmU * SmA * SmU * SAMC6T * | | |
| | | SmU | | |
| WV- | TUUUAAGCAACCUACAGGGG | T * fU * mUfU * mAfA * mGfC * mAfA * mCfC | XXOXOXOXOXOXOXXXXXX | 1746 |
| 10683 | CTU | * mUfA * mC * fA * mG * fG * mG * fG * mC * | XXX | |
| | | T * mU | | |
| WV- | TUUUUAAGCAACCUACAGGG | T * fU * mUfU * mUfA * mAfG * mCfA * mAfC | XXOXOXOXOXOXOXXXXXX | 1747 |
| 10684 | GTU | * mCfU * mA * fC * mA * fG * mG * fG * mG * | XXX | |
| | | T * mU | | |
| WV- | TCUUUUAAGCAACCUACAGG | T * fC * mUfU * mUfU * mAfA * mGfC * mAfA | XXOXOXOXOXOXOXXXXXX | 1748 |
| 10685 | GTU | * mCfC * mU * fA * mC * fA * mG * fG * mG * | XXX | |
| | | T * mU | | |
| WV- | TCCUUUUAAGCAACCUACAG | T * fC * mCfU * mUfU * mUfA * mAfG * mCfA | XXOXOXOXOXOXOXXXXXX | 1749 |
| 10686 | GTU | * mAfC * mC * fU * mA * fC * mA * fG * mG * | XXX | |
| | | T * mU | | |
| WV- | TCCCUUUUAAGCAACCUACA | T * fC * mCfC * mUfU * mUfU * mAfA * mGfC | XXOXOXOXOXOXOXXXXXX | 1750 |
| 10687 | GTU | * mAfA * mC * fC * mU * fA * mC * fA * mG * | XXX | |
| | | T * mU | | |
| WV- | TUCCCUUUUAAGCAACCUAC | T * fU * mCfC * mCfU * mUfU * mUfA * mAfG | XXOXOXOXOXOXOXXXXXX | 1751 |
| 10688 | ATU | * mCfA * mA * fC * mC * fU * mA * fC * mA * | XXX | |
| | | T * mU | | |
| WV- | TGUCCCUUUUAAGCAACCUA | T * fG * mUfC * mCfC * mUfU * mUfU * mAfA | XXOXOXOXOXOXOXXXXXX | 1752 |
| 10689 | CTU | * mGfC * mA * fA * mC * fC * mU * fA * mC * | XXX | |
| | | T * mU | | |
| WV- | TUGUCCCUUUUAAGCAACCU | T * fU * mGfU * mCfC * mCfU * mUfU * mUfA | XXOXOXOXOXOXOXXXXXX | 1753 |
| 10690 | ATU | * mAfG * mC * fA * mA * fC * mC * fU * mA * | XXX | |
| | | T * mU | | |
| WV- | TCUGUCCCUUUUAAGCAACC | T * fC * mUfG * mUfC * mCfC * mUfU * mUfU | XXOXOXOXOXOXOXXXXXX | 1754 |
| 10691 | UTU | * mAfA * mG * fC * mA * fA * mC * fC * mU * | XXX | |
| | | T * mU | | |
| WV- | TACUGUCCCUUUUAAGCAAC | T * fA * mCfU * mGfU * mCfC * mCfU * mUfU | XXOXOXOXOXOXOXXXXXX | 1755 |
| 10692 | CTU | * mUfA * mA * fG * mC * fA * mA * fC * mC * | XXX | |
| | | T * mU | | |
| WV- | TUACUGUCCCUUUUAAGCAA | T * fU * mAfC * mUfG * mUfC * mCfC * mUfU | XXOXOXOXOXOXOXXXXXX | 1756 |
| 10693 | CTU | * mUfU * mA * fA * mG * fC * mA * fA * mC * | XXX | |
| | | T * mU | | |
| WV- | TAUACUGUCCCUUUUAAGCA | T * fA * mUfA * mCfU * mGfU * mCfC * mCfU | XXOXOXOXOXOXOXXXXXX | 1757 |
| 10694 | ATU | * mUfU * mU * fA * mA * fG * mC * fA * mA * | XXX | |
| | | T * mU | | |
| WV- | TAAUACUGUCCCUUUUAAGC | T * fA * mAfU * mAfC * mUfG * mUfC * mCfC | XXOXOXOXOXOXOXXXXXX | 1758 |
| 10695 | ATU | * mUfU * mU * fU * mA * fA * mG * fC * mA * | | |
| | | XXX | | |
| | | T * mU | | |
| WV- | TGAAUACUGUCCCUUUUAAG | T * fG * mAfA * mUfA * mCfU * mGfU * mCfC | XXOXOXOXOXOXOXXXXXX | 1759 |
| 10696 | CTU | * mCfU * mU * fU * mU * fA * mA * fG * mC * | XXX | |
| | | T * mU | | |
| WV- | TAGAAUACUGUCCCUUUUAA | T * fA * mGfA * mAfU * mAfC * mUfG * mUfC | XXOXOXOXOXOXOXXXXXX | 1760 |
| 10697 | GTU | * mCfC * mU * fU * mU * fU * mA * fA * mG * | XXX | |
| | | T * mU | | |
| WV- | TGAGAAUACUGUCCCUUUUA | T * fG * mAfG * mAfA * mUfA * mCfU * mGfU | XXOXOXOXOXOXOXXXXXX | 1761 |
| 10698 | ATU | * mCfC * mC * fU * mU * fU * mU * fA * mA * | XXX | |
| | | T * mU | | |
| WV- | TUGAGAAUACUGUCCCUUUU | T * fU * mGfA * mGfA * mAfU * mAfC * mUfG | XXOXOXOXOXOXOXXXXXX | 1762 |
| 10699 | ATU | * mUfC * mC * fC * mU * fU * mU * fU * mA * | XXX | |
| | | T * mU | | |
| WV- | TCUGAGAAUACUGUCCCUUU | T * fC * mUfG * mAfG * mAfA * mUfA * mCfU | XXOXOXOXOXOXOXXXXXX | 1763 |
| 10700 | UTU | * mGfU * mC * fC * mC * fU * mU * fU * mU * | XXX | |
| | | T * mU | | |
| WV- | TACUGAGAAUACUGUCCCUU | T * fA * mCfU * mGfA * mGfA * mAfU * mAfC | XXOXOXOXOXOXOXXXXXX | 1764 |
| 10701 | UTU | * mUfG * mU * fC * mC * fC * mU * fU * mU * | XXX | |
| | | T * mU | | |
| WV- | TGCACUGAGAAUACUGUCCC | T * fG * mCfA * mCfU * mGfA * mGfA * mAfU | XXOXOXOXOXOXOXXXXXX | 1765 |
| 10702 | UTU | * mAfC * mU * fG * mU * fC * mC * fC * mU * | XXX | |
| | | T * mU | | |
| WV- | TAGCACUGAGAAUACUGUCC | T * fA * mGfC * mAfC * mUfG * mAfG * mAfA | XXOXOXOXOXOXOXXXXXX | 1766 |
| 10703 | CTU | * mUfA * mC * fU * mG * fU * mC * fC * mC * | XXX | |
| | | T * mU | | |
| WV- | TGAGCACUGAGAAUACUGUC | T * fG * mAfG * mCfA * mCfU * mGfA * mGfA | XXOXOXOXOXOXOXXXXXX | 1767 |
| 10704 | CTU | * mAfU * mA * fC * mU * fG * mU * fC * mC * | XXX | |
| | | T * mU | | |
| WV- | TAGAGCACUGAGAAUACUGU | T * fA * mGfA * mGfC * mAfC * mUfG * mAfG | XXOXOXOXOXOXOXXXXXX | 1768 |
| 10705 | CTU | * mAfA * mU * fA * mC * fU * mG * fU * mC * | XXX | |
| | | T * mU | | |
| WV- | TGAGAGCACUGAGAAUACUG | T * fG * mAfG * mAfG * mCfA * mCfU * mGfA | XXOXOXOXOXOXOXXXXXX | 1769 |
| 10706 | UTU | * mGfA * mA * fU * mA * fC * mU * fG * mU * | XXX | |
| | | T * mU | | |
| WV- | TGGAGAGCACUGAGAAUACU | T * fG * mGfA * mGfA * mGfC * mAfC * mUfG | XXOXOXOXOXOXOXXXXXX | 1770 |
| 10707 | GTU | * mAfG * mA * fA * mU * fA * mC * fU * mG * | XXX | |
| | | T * mU | | |
| WV- | TAGGAGAGCACUGAGAAUAC | T * fA * mGfG * mAfG * mAfG * mCfA * mCfU | XXOXOXOXOXOXOXXXXXX | 1771 |
| 10708 | UTU | * mGfA * mG * fA * mA * fU * mA * fC * mU * | XXX | |
| | | T * mU | | |
| WV- | TUAGGAGAGCACUGAGAAUA | T * fU * mAfG * mGfA * mGfA * mGfC * mAfC | XXOXOXOXOXOXOXXXXXX | 1772 |
| 10709 | CTU | * mUfG * mA * fG * mA * fA * mU * fA * mC * | XXX | |
| | | T * mU | | |
| WV- | TGUAGGAGAGCACUGAGAAU | T * fG * mUfA * mGfG * mAfG * mAfG * mCfA | XXOXOXOXOXOXOXXXXXX | 1773 |
| 10710 | ATU | * mCfU * mG * fA * mG * fA * mA * fU * mA * | XXX | |
| | | T * mU | | |
| WV- | TGGUAGGAGAGCACUGAGAA | T * fG * mGfU * mAfG * mGfA * mGfA * mGfC | XXOXOXOXOXOXOXXXXXX | 1774 |
| 10711 | UTU | * mAfC * mU * fG * mA * fG * mA * fA * mU * | XXX | |
| | | T * mU | | |
| WV- | TGGGUAGGAGAGCACUGAGA | T * fG * mGfG * mUfA * mGfG * mAfG * mAfG | XXOXOXOXOXOXOXXXXXX | 1775 |
| 10712 | ATU | * mCfA * mC * fU * mG * fA * mG * fA * mA * | XXX | |
| | | T * mU | | |
| WV- | TGGGGUAGGAGAGCACUGAG | T * fG * mGfG * mGfU * mAfG * mGfA * mGfA | XXOXOXOXOXOXOXXXXXX | 1776 |
| 10713 | ATU | * mGfC * mA * fC * mU * fG * mA * fG * mA * | XXX | |
| | | T * mU | | |
| WV- | TUGGGGUAGGAGAGCACUGA | T * fU * mGfG * mGfG * mUfA * mGfG * mAfG | XXOXOXOXOXOXOXXXXXX | 1777 |
| 10714 | GTU | * mAfG * mC * fA * mC * fU * mG * fA * mG * | XXX | |
| | | T * mU | | |
| WV- | TGUGGGGUAGGAGAGCACU | T * fG * mUfG * mGfG * mGfU * mAfG * mGfA | XXOXOXOXOXOXOXXXXXX | 1778 |
| 10715 | GATU | * mGfA * mG * fC * mA * fC * mU * fG * mA * | XXX | |
| | | T * mU | | |
| WV- | TGGUGGGGUAGGAGAGCAC | T * fG * mGfU * mGfG * mGfG * mUfA * mGfG | XXOXOXOXOXOXOXXXXXX | 1779 |
| 10716 | UGTU | * mAfG * mA * fG * mC * fA * mC * fU * mG * | XXX | |
| | | T * mU | | |
| WV- | TAGGUGGGGUAGGAGAGCAC | T * fA * mGfG * mUfG * mGfG * mGfU * mAfG | XXOXOXOXOXOXOXXXXXX | 1780 |
| 10717 | UTU | * mGfA * mG * fA * mG * fC * mA * fC * mU * | XXX | |
| | | T * mU | | |
| WV- | TGAGGUGGGGUAGGAGAGC | T * fG * mAfG * mGfU * mGfG * mGfG * mUfA | XXOXOXOXOXOXOXXXXXX | 1781 |
| 10718 | ACTU | * mGfG * mA * fG * mA * fG * mC * fA * mC * | XXX | |
| | | T * mU | | |
| WV- | TUGAGGUGGGGUAGGAGAG | T * fU * mGfA * mGfG * mUfG * mGfG * mGfU | XXOXOXOXOXOXOXXXXXX | 1782 |
| 10719 | CATU | * mAfG * mG * fA * mG * fA * mG * fC * mA * | XXX | |
| | | T * mU | | |
| WV- | TAUGAGGUGGGGUAGGAGA | T * fA * mUfG * mAfG * mGfU * mGfG * mGfG | XXOXOXOXOXOXOXXXXXX | 1783 |
| 10720 | GCTU | * mUfA * mG * fG * mA * fG * mA * fG * mC * | XXX | |
| | | T * mU | | |
| WV- | TCAUGAGGUGGGGUAGGAG | T * fC * mAfU * mGfA * mGfG * mUfG * mGfG | XXOXOXOXOXOXOXXXXXX | 1784 |
| 10721 | AGTU | * mGfU * mA * fG * mG * fA * mG * fA * mG * | XXX | |
| | | T * mU | | |
| WV- | TGCAUGAGGUGGGGUAGGA | T * fG * mCfA * mUfG * mAfG * mGfU * mGfG | XXOXOXOXOXOXOXXXXXX | 1785 |
| 10722 | GATU | * mGfG * mU * fA * mG * fG * mA * fG * mA * | XXX | |
| | | T * mU | | |
| WV- | TUUAAGCAACCUACAGGGGC | T * fU * mUmA * mAmG * mCmA * mAmC * | XXOXOXOXOXOXOXXXXXX | 1786 |
| 10723 | ATU | mCmU * mAfC * mA * mG * mG * mG * mG * | XXX | |
| | | mC * mA * T * mU | | |
| WV- | TUUUAAGCAACCUACAGGGG | T * fU * mUmU * mAmA * mGmC * mAmA * | XXOXOXOXOXOXOXXXXXX | 1787 |
| 10724 | CTU | mCmC * mUfA * mC * mA * mG * mG * mG * | XXX | |
| | | mG * mC * T * mU | | |
| WV- | TUUUUAAGCAACCUACAGGG | T * fU * mUmU * mUmA * mAmG * mCmA * | XXOXOXOXOXOXOXXXXXX | 1788 |
| 10725 | GTU | mAmC * mCfU * mA * mC * mA * mG * mG * | XXX | |
| | | mG * mG * T * mU | | |
| WV- | TCUUUUAAGCAACCUACAGG | T * fC * mUmU * mUmU * mAmA * mGmC * | XXOXOXOXOXOXOXXXXXX | 1789 |
| 10726 | GTU | mAmA * mCfC * mU * mA * mC * mA * mG * | XXX | |
| | | mG * mG * T * mU | | |
| WV- | TCCUUUUAAGCAACCUACAG | T * fC * mCmU * mUmU * mUmA * mAmG * | XXOXOXOXOXOXOXXXXXX | 1790 |
| 10727 | GTU | mCmA * mAfC * mC * mU * mA * mC * mA * | XXX | |
| | | mG * mG * T * mU | | |
| WV- | TCCCUUUUAAGCAACCUACA | T * fC * mCmC * mUmU * mUmU * mAmA * | XXOXOXOXOXOXOXXXXXX | 1791 |
| 10728 | GTU | mGmC * mAfA * mC * mC * mU * mA * mC * | XXX | |
| | | mA * mG * T * mU | | |
| WV- | TUCCCUUUUAAGCAACCUAC | T * fU * mCmC * mCmU * mUmU * mUmA * | XXOXOXOXOXOXOXXXXXX | 1792 |
| 10729 | ATU | mAmG * mCfA * mA * mC * mC * mU * mA * | XXX | |
| | | mC * mA * T * mU | | |
| WV- | TGUCCCUUUUAAGCAACCUA | T * fG * mUmC * mCmC * mUmU * mUmU * | XXOXOXOXOXOXOXXXXXX | 1793 |
| 10730 | CTU | mAmA * mGfC * mA * mA * mC * mC * mU * | XXX | |
| | | mA * mC * T * mU | | |
| WV- | TUGUCCCUUUUAAGCAACCU | T * fU * mGmU * mCmC * mCmU * mUmU * | XXOXOXOXOXOXOXXXXXX | 1794 |
| 10731 | ATU | mUmA * mAfG * mC * mA * mA * mC * mC * | XXX | |
| | | mU * mA * T * mU | | |
| WV- | TCUGUCCCUUUUAAGCAACC | T * fC * mUmG * mUmC * mCmC * mUmU * | XXOXOXOXOXOXOXXXXXX | 1795 |
| 10732 | UTU | mUmU * mAfA * mG * mC * mA * mA * mC * | XXX | |
| | | mC * mU * T * mU | | |
| WV- | TACUGUCCCUUUUAAGCAAC | T * fA * mCmU * mGmU * mCmC * mCmU * | XXOXOXOXOXOXOXXXXXX | 1796 |
| 10733 | CTU | mUmU * mUfA * mA * mG * mC * mA * mA * | XXX | |
| | | mC * mC * T * mU | | |
| WV- | TUACUGUCCCUUUUAAGCAA | T * fU * mAmC * mUmG * mUmC * mCmC * | XXOXOXOXOXOXOXXXXXX | 1797 |
| 10734 | CTU | mUmU * mUfU * mA * mA * mG * mC * mA * | XXX | |
| | | mA * mC * T * mU | | |
| WV- | TAUACUGUCCCUUUUAAGCA | T * fA * mUmA * mCmU * mGmU * mCmC * | XXOXOXOXOXOXOXXXXXX | 1798 |
| 10735 | ATU | mCmU * mUfU * mU * mA * mA * mG * mC * | XXX | |
| | | mA * mA * T * mU | | |
| WV- | TAAUACUGUCCCUUUUAAGC | T * fA * mAmU * mAmC * mUmG * mUmC * | XXOXOXOXOXOXOXXXXXX | 1799 |
| 10736 | ATU | mCmC * mUfU * mU * mU * mA * mA * mG * | XXX | |
| | | mC * mA * T * mU | | |
| WV- | TGAAUACUGUCCCUUUUAAG | T * fG * mAmA * mUmA * mCmU * mGmU * | XXOXOXOXOXOXOXXXXXX | 1800 |
| 10737 | CTU | mCmC * mCfU * mU * mU * mU * mA * mA * | XXX | |
| | | mG * mC * T * mU | | |
| WV- | TAGAAUACUGUCCCUUUUAA | T * fA * mGmA * mAmU * mAmC * mUmG * | XXOXOXOXOXOXOXXXXXX | 1801 |
| 10738 | GTU | mUmC * mCfC * mU * mU * mU * mU * mA * | XXX | |
| | | mA * mG * T * mU | | |
| WV- | TGAGAAUACUGUCCCUUUUA | T * fG * mAmG * mAmA * mUmA * mCmU * | XXOXOXOXOXOXOXXXXXX | 1802 |
| 10739 | ATU | mGmU * mCfC * mC * mU * mU * mU * mU * | XXX | |
| | | mA * mA * T * mU | | |
| WV- | TUGAGAAUACUGUCCCUUUU | T * fU * mGmA * mGmA * mAmU * mAmC * | XXOXOXOXOXOXOXXXXXX | 1803 |
| 10740 | ATU | mUmG * mUfC * mC * mC * mU * mU * mU * | XXX | |
| | | mU * mA * T * mU | | |
| WV- | TCUGAGAAUACUGUCCCUUU | T * fC * mUmG * mAmG * mAmA * mUmA * | XXOXOXOXOXOXOXXXXXX | 1804 |
| 10741 | UTU | mCmU * mGfU * mC * mC * mC * mU * mU * | XXX | |
| | | mU * mU * T * mU | | |
| WV- | TACUGAGAAUACUGUCCCUU | T * fA * mCmU * mGmA * mGmA * mAmU * | XXOXOXOXOXOXOXXXXXX | 1805 |
| 10742 | UTU | mAmC * mUfG * mU * mC * mC * mC * mU * | XXX | |
| | | mU * mU * T * mU | | |
| WV- | TGCACUGAGAAUACUGUCCC | T * fG * mCmA * mCmU * mGmA * mGmA * | XXOXOXOXOXOXOXXXXXX | 1806 |
| 10743 | UTU | mAmU * mAfC * mU * mG * mU * mC * mC * | XXX | |
| | | mC * mU * T * mU | | |
| WV- | TAGCACUGAGAAUACUGUCC | T * fA * mGmC * mAmC * mUmG * mAmG * | XXOXOXOXOXOXOXXXXXX | 1807 |
| 10744 | CTU | mAmA * mUfA * mC * mU * mG * mU * mC * | XXX | |
| | | mC * mC * T * mU | | |
| WV- | TGAGCACUGAGAAUACUGUC | T * fG * mAmG * mCmA * mCmU * mGmA * | XXOXOXOXOXOXOXXXXXX | 1808 |
| 10745 | CTU | mGmA * mAfU * mA * mC * mU * mG * mU * | XXX | |
| | | mC * mC * T * mU | | |
| WV- | TAGAGCACUGAGAAUACUGU | T * fA * mGmA * mGmC * mAmC * mUmG * | XXOXOXOXOXOXOXXXXXX | 1809 |
| 10746 | CTU | mAmG * mAfA * mU * mA * mC * mU * mG * | XXX | |
| | | mU * mC * T * mU | | |
| WV- | TGAGAGCACUGAGAAUACUG | T * fG * mAmG * mAmG * mCmA * mCmU * | XXOXOXOXOXOXOXXXXXX | 1810 |
| 10747 | UTU | mGmA * mGfA * mA * mU * mA * mC * mU * | XXX | |
| | | mG * mU * T * mU | | |
| WV- | TGGAGAGCACUGAGAAUACU | T * fG * mGmA * mGmA * mGmC * mAmC * | XXOXOXOXOXOXOXXXXXX | 1811 |
| 10748 | GTU | mUmG * mAfG * mA * mA * mU * mA * mC * | XXX | |
| | | mU * mG * T * mU | | |
| WV- | TAGGAGAGCACUGAGAAUAC | T * fA * mGmG * mAmG * mAmG * mCmA * | XXOXOXOXOXOXOXXXXXX | 1812 |
| 10749 | UTU | mCmU * mGfA * mG * mA * mA * mU * mA * | XXX | |
| | | mC * mU * T * mU | | |
| WV- | TUAGGAGAGCACUGAGAAUA | T * fU * mAmG * mGmA * mGmA * mGmC * | XXOXOXOXOXOXOXXXXXX | 1813 |
| 10750 | CTU | mAmC * mUfG * mA * mG * mA * mA * mU * | XXX | |
| | | mA * mC * T * mU | | |
| WV- | TGUAGGAGAGCACUGAGAAU | T * fG * mUmA * mGmG * mAmG * mAmG * | XXOXOXOXOXOXOXXXXXX | 1814 |
| 10751 | ATU | mCmA * mCfU * mG * mA * mG * mA * mA * | XXX | |
| | | mU * mA * T * mU | | |
| WV- | TGGUAGGAGAGCACUGAGAA | T * fG * mGmU * mAmG * mGmA * mGmA * | XXOXOXOXOXOXOXXXXXX | 1815 |
| 10752 | UTU | mGmC * mAfC * mU * mG * mA * mG * mA * | XXX | |
| | | mA * mU * T * mU | | |
| WV- | TGGGUAGGAGAGCACUGAGA | T * fG * mGmG * mUmA * mGmG * mAmG * | XXOXOXOXOXOXOXXXXXX | 1816 |
| 10753 | ATU | mAmG * mCfA * mC * mU * mG * mA * mG * | XXX | |
| | | mA * mA * T * mU | | |
| WV- | TGGGGUAGGAGAGCACUGAG | T * fG * mGmG * mGmU * mAmG * mGmA * | XXOXOXOXOXOXOXXXXXX | 1817 |
| 10754 | ATU | mGmA * mGfC * mA * mC * mU * mG * mA * | XXX | |
| | | mG * mA * T * mU | | |
| WV- | TUGGGGUAGGAGAGCACUGA | T * fU * mGmG * mGmG * mUmA * mGmG * | XXOXOXOXOXOXOXXXXXX | 1818 |
| 10755 | GTU | mAmG * mAfG * mC * mA * mC * mU * mG * | XXX | |
| | | mA * mG * T * mU | | |
| WV- | TGUGGGGUAGGAGAGCACU | T * fG * mUmG * mGmG * mGmU * mAmG * | XXOXOXOXOXOXOXXXXXX | 1819 |
| 10756 | GATU | mGmA * mGfA * mG * mC * mA * mC * mU * | XXX | |
| | | mG * mA * T * mU | | |
| WV- | TGGUGGGGUAGGAGAGCAC | T * fG * mGmU * mGmG * mGmG * mUmA * | XXOXOXOXOXOXOXXXXXX | 1820 |
| 10757 | UGTU | mGmG * mAfG * mA * mG * mC * mA * mC * | XXX | |
| | | mU * mG * T * mU | | |
| WV- | TAGGUGGGGUAGGAGAGCAC | T * fA * mGmG * mUmG * mGmG * mGmU * | XXOXOXOXOXOXOXXXXXX | 1821 |
| 10758 | UTU | mAmG * mGfA * mG * mA * mG * mC * mA * | XXX | |
| | | mC * mU * T * mU | | |
| WV- | TGAGGUGGGGUAGGAGAGC | T * fG * mAmG * mGmU * mGmG * mGmG * | XXOXOXOXOXOXOXXXXXX | 1822 |
| 10759 | ACTU | mUmA * mGfG * mA * mG * mA * mG * mC * | XXX | |
| | | mA * mC * T * mU | | |
| WV- | TUGAGGUGGGGUAGGAGAG | T * fU * mGmA * mGmG * mUmG * mGmG * | XXOXOXOXOXOXOXXXXXX | 1823 |
| 10760 | CATU | mGmU * mAfG * mG * mA * mG * mA * mG * | XXX | |
| | | mC * mA * T * mU | | |
| WV- | TAUGAGGUGGGGUAGGAGA | T * fA * mUmG * mAmG * mGmU * mGmG * | XXOXOXOXOXOXOXXXXXX | 1824 |
| 10761 | GCTU | mGmG * mUfA * mG * mG * mA * mG * mA * | XXX | |
| | | mG * mC * T * mU | | |
| WV- | TCAUGAGGUGGGGUAGGAG | T * fC * mAmU * mGmA * mGmG * mUmG * | XXOXOXOXOXOXOXXXXXX | 1825 |
| 10762 | AGTU | mGmG * mGfU * mA * mG * mG * mA * mG * | XXX | |
| | | mA * mG * T * mU | | |
| WV- | TGCAUGAGGUGGGGUAGGA | T * fG * mCmA * mUmG * mAmG * mGmU * | XXOXOXOXOXOXOXXXXXX | 1826 |
| 10763 | GATU | mGmG * mGfG * mU * mA * mG * mG * mA * | XXX | |
| | | mG * mA * T * mU | | |
| WV- | TUUAAGCAACCUACAGGGGC | T * fU * mUfA * mAfG * mCfA * mAfC * mCfU | XXOXOXOXOXOXOXXXXXX | 1827 |
| 10764 | ATU | * mAfC * mA * fG * mG * fG * mG * fC * mA * | XXX | |
| | | T * mU | | |
| WV- | TGCCACUGUAGAAAGGCAUG | PO5MRdT * SfG * SmC * SmC * SmAmC * | SSSSOSOSOSOSOSSSSSSSS | 1828 |
| 12499 | ATU | SmUmG * SmUmA * SmGmA * SmAfA * SmG * | S | |
| | | SmG * SmC * SmA * SmU * SmG * SmA * | | |
| | | STGaNC6T * SmU | | |
| WV- | TGCCACUGUAGAAAGGCAUG | PO5MRdT * SfG * SmC * SmC * SmAmC * | SSSSOSOSOSOSOSSSSSSSS | 1829 |
| 12500 | ATU | SmUmG * SmUmA * SmGmA * SmAfA * SmG * | S | |
| | | SmG * SmC * SmA * SmU * SmG * SmA * | | |
| | | SAMC6T * SmU | | |
| WV- | TGCCACUGUAGAAAGGCAUG | PO5MRdT * SfG * SmC5MSmC * SmAmC * | SSOSOSOSOSOSOSSSSSSSS | 1830 |
| 12501 | ATU | SmUmG * SmUmA * SmGmA * SmAfA * SmG * | S | |
| | | SmG * SmC * SmA * SmU * SmG * SmA * | | |
| | | STGaNC6T * SmU | | |
| WV- | TGCCACUGUAGAAAGGCAUG | PO5MRdT * SfG * SmC5MSmC * SmAmC * | SSOSOSOSOSOSOSSSSSSSS | 1831 |
| 12502 | ATU | SmUmG * SmUmA * SmGmA * SmAfA * SmG * | S | |
| | | SmG * SmC * SmA * SmU * SmG * SmA * | | |
| | | SAMC6T * SmU | | |
| WV- | GCACTGAGAATACTGTCCCT | Geo * m5CeoAeom5CeoTeo * G * A * G * A * | XOOOXXXXXXXXXXXOOOX | 1832 |
| 12918 | | A * T * A * C * T * G * | | |
| | | Teom5Ceom5Ceom5Ceo * Teo | | |
| WV- | CACTGAGAATACTGTCCCTT | m5Ceo * Aeom5CeoTeoGeo * A * G * A * A * T | XOOOXXXXXXXXXXXOOOX | 1833 |
| 12919 | | * A * C * T * G * T * m5Ceom5Ceom5CeoTeo * | | |
| | | Teo | | |
| WV- | ACTGAGAATACTGTCCCTTT | Aeo * m5CeoTeoGeoAeo * G * A * A * T * A * | XOOOXXXXXXXXXXXOOOX | 1834 |
| 12920 | | C * T * G * T * C * m5Ceom5CeoTeoTeo * Teo | | |
| WV- | CTGAGAATACTGTCCCTTTT | m5Ceo * TeoGeoAeoGeo * A * A * T * A * C * | XOOOXXXXXXXXXXXOOOX | 1835 |
| 12921 | | T * G * T * C * C * m5CeoTeoTeoTeo * Teo | | |
| WV- | TGAGAATACTGTCCCTTTTA | Teo * GeoAeoGeoAeo * A * T * A * C * T * G * | XOOOXXXXXXXXXXXOOOX | 1836 |
| 12922 | | T * C * C * C * TeoTeoTeoTeo * Aeo | | |
| WV- | GAGAATACTGTCCCTTTTAA | Geo * AeoGeoAeoAeo * T * A * C * T * G * T * | XOOOXXXXXXXXXXXOOOX | 1837 |
| 12923 | | C * C * C * T * TeoTeoTeoAeo * Aeo | | |
| WV- | AGAATACTGTCCCTTTTAAG | Aeo * GeoAeoAeoTeo * A * C * T * G * T * C * | XOOOXXXXXXXXXXXOOOX | 1838 |
| 12924 | | C * C * T * T * TeoTeoAeoAeo * Geo | | |
| WV- | GAATACTGTCCCTTTTAAGC | Geo * AeoAeoTeoAeo * C * T * G * T * C * C * | XOOOXXXXXXXXXXXOOOX | 1839 |
| 12925 | | C * T * T * T * TeoAeoAeoGeo * m5Ceo | | |
| WV- | AATACTGTCCCTTTTAAGCA | Aeo * AeoTeoAeom5Ceo * T * G * T * C * C * C | XOOOXXXXXXXXXXXOOOX | 1840 |
| 12926 | | * T * T * T * T * AeoAeoGeom5Ceo * Aeo | | |
| WV- | ATACTGTCCCTTTTAAGCAA | Aeo * TeoAeom5CeoTeo * G * T * C * C * C * T | XOOOXXXXXXXXXXXOOOX | 1841 |
| 12927 | | * T * T * T * A * AeoGeom5CeoAeo * Aeo | | |
| WV- | TACTGTCCCTTTTAAGCAAC | Teo * Aeom5CeoTeoGeo * T * C * C * C * T * T | XOOOXXXXXXXXXXXOOOX | 1842 |
| 12928 | | * T * T * A * A * Geom5CeoAeoAeo * m5Ceo | | |
| WV- | ACTGTCCCTTTTAAGCAACC | Aeo * m5CeoTeoGeoTeo * C * C * C * T * T * T | XOOOXXXXXXXXXXXOOOX | 1843 |
| 12929 | | * T * A * A * G * m5CeoAeoAeom5Ceo * | | |
| | | m5Ceo | | |
| WV- | CTGTCCCTTTTAAGCAACCT | m5Ceo * TeoGeoTeom5Ceo * C * C * T * T * T | XOOOXXXXXXXXXXXOOOX | 1844 |
| 12930 | | * T * A * A * G * C * AeoAeom5Ceom5Ceo * | | |
| | | Teo | | |
| WV- | TGTCCCTTTTAAGCAACCTA | Teo * GeoTeom5Ceom5Ceo * C * T * T * T * T | XOOOXXXXXXXXXXXOOOX | 1845 |
| 12931 | | * A * A * G * C * A * Aeom5Ceom5CeoTeo * | | |
| | | Aeo | | |
| WV- | GTCCCTTTTAAGCAACCTAC | Geo * Teom5Ceom5Ceom5Ceo * T * T * T * T | XOOOXXXXXXXXXXXOOOX | 1846 |
| 12932 | | * A * A * G * C * A * A * m5Ceom5CeoTeoAeo | | |
| | | * m5Ceo | | |
| WV- | TCCCTTTTAAGCAACCTACA | Teo * m5Ceom5Ceom5CeoTeo * T * T * T * A | XOOOXXXXXXXXXXXOOOX | 1847 |
| 12933 | | * A * G * C * A * A * C * m5CeoTeoAeom5Ceo | | |
| | | * Aeo | | |
| WV- | CCCTTTTAAGCAACCTACAG | m5Ceo * m5Ceom5CeoTeoTeo * T * T * A * A | XOOOXXXXXXXXXXXOOOX | 1848 |
| 12934 | | * G * C * A * A * C * C * TeoAeom5CeoAeo * | | |
| | | Geo | | |
| WV- | CCTTTTAAGCAACCTACAGG | m5Ceo * m5CeoTeoTeoTeo * T * A * A * G * C | XOOOXXXXXXXXXXXOOOX | 1849 |
| 12935 | | * A * A * C * C * T * Aeom5CeoAeoGeo * Geo | | |
| WV- | CTTTTAAGCAACCTACAGGG | m5Ceo * TeoTeoTeoTeo * A * A * G * C * A * A | XOOOXXXXXXXXXXXOOOX | 1850 |
| 12936 | | * C * C * T * A * m5CeoAeoGeoGeo * Geo | | |
| WV- | TTTTAAGCAACCTACAGGGG | Teo * TeoTeoTeoAeo * A * G * C * A * A * C * | XOOOXXXXXXXXXXXOOOX | 1851 |
| 12937 | | C * T * A * C * AeoGeoGeoGeo * Geo | | |
| WV- | TTTAAGCAACCTACAGGGGC | Teo * TeoTeoAeoAeo * G * C * A * A * C * C * | XOOOXXXXXXXXXXXOOOX | 1852 |
| 12938 | | T * A * C * A * GeoGeoGeoGeo * m5Ceo | | |
| WV- | TTAAGCAACCTACAGGGGCA | Teo * TeoAeoAeoGeo * C * A * A * C * C * T * | XOOOXXXXXXXXXXXOOOX | 1853 |
| 12939 | | A * C * A * G * GeoGeoGeom5Ceo * Aeo | | |
| WV- | TAAGCAACCTACAGGGGCAG | Teo * AeoAeoGeom5Ceo * A * A * C * C * T * | XOOOXXXXXXXXXXXOOOX | 1854 |
| 12940 | | A * C * A * G * G * GeoGeom5CeoAeo * Geo | | |
| WV- | AAGCAACCTACAGGGGCAGC | Aeo * AeoGeom5CeoAeo * A * C * C * T * A * | XOOOXXXXXXXXXXXOOOX | 1855 |
| 12941 | | C * A * G * G * G * Geom5CeoAeoGeo * | | |
| | | m5Ceo | | |
| WV- | AGCAACCTACAGGGGCAGCC | Aeo * Geom5CeoAeoAeo * C * C * T * A * C * | XOOOXXXXXXXXXXXOOOX | 1856 |
| 12942 | | A * G * G * G * G * m5CeoAeoGeom5Ceo * | | |
| | | m5Ceo | | |
| WV- | GCAACCTACAGGGGCAGCCC | Geo * m5CeoAeoAeom5Ceo * C * T * A * C * A | XOOOXXXXXXXXXXXOOOX | 1857 |
| 12943 | | * G * G * G * G * C * AeoGeom5Ceom5Ceo * | | |
| | | m5Ceo | | |
| WV- | CAACCTACAGGGGCAGCCCT | m5Ceo * AeoAeom5Ceom5Ceo * T * A * C * A | XOOOXXXXXXXXXXXOOOX | 1858 |
| 12944 | | * G * G * G * G * C * A * | | |
| | | Geom5Ceom5Ceom5Ceo * Teo | | |
| WV- | AACCTACAGGGGCAGCCCTG | Aeo * Aeom5Ceom5CeoTeo * A * C * A * G * | XOOOXXXXXXXXXXXOOOX | 1859 |
| 12945 | | G * G * G * C * A * G * | | |
| | | m5Ceom5Ceom5CeoTeo * Geo | | |
| WV- | ACCTACAGGGGCAGCCCTGG | Aeo * m5Ceom5CeoTeoAeo * C * A * G * G * | XOOOXXXXXXXXXXXOOOX | 1860 |
| 12946 | | G * G * C * A * G * C * m5Ceom5CeoTeoGeo * | | |
| | | Geo | | |
| WV- | GCACTGAGAATACTGUCCCU | Geo * m5CeoAeom5CeoTeo * G * A * G * A * | XOOOXXXXXXXXXXXXXXX | 1861 |
| 12947 | | A * T * A * C * T * G * mU * mC * mC * mC * | | |
| | | mU | | |
| WV- | CACTGAGAATACTGTCCCUU | m5Ceo * Aeom5CeoTeoGeo * A * G * A * A * T | XOOOXXXXXXXXXXXXXXX | 1862 |
| 12948 | | * A * C * T * G * T * mC * mC * mC * mU * mU | | |
| WV- | ACTGAGAATACTGTCCCUUU | Aeo * m5CeoTeoGeoAeo * G * A * A * T * A * | XOOOXXXXXXXXXXXXXXX | 1863 |
| 12949 | | C * T * G * T * C * mC * mC * mU * mU * mU | | |
| WV- | CTGAGAATACTGTCCCUUUU | m5Ceo * TeoGeoAeoGeo * A * A * T * A * C * | XOOOXXXXXXXXXXXXXXX | 1864 |
| 12950 | | T * G * T * C * C * mC * mU * mU * mU * mU | | |
| WV- | TGAGAATACTGTCCCUUUUA | Teo * GeoAeoGeoAeo * A * T * A * C * T * G * | XOOOXXXXXXXXXXXXXXX | 1865 |
| 12951 | | T * C * C * C * mU * mU * mU * mU * mA | | |
| WV- | GAGAATACTGTCCCTUUUAA | Geo * AeoGeoAeoAeo * T * A * C * T * G * T * | XOOOXXXXXXXXXXXXXXX | 1866 |
| 12952 | | C * C * C * T * mU * mU * mU * mA * mA | | |
| WV- | AGAATACTGTCCCTTUUAAG | Aeo * GeoAeoAeoTeo * A * C * T * G * T * C * | XOOOXXXXXXXXXXXXXXX | 1867 |
| 12953 | | C * C * T * T * mU * mU * mA * mA * mG | | |
| WV- | GAATACTGTCCCTTTUAAGC | Geo * AeoAeoTeoAeo * C * T * G * T * C * C * | XOOOXXXXXXXXXXXXXXX | 1868 |
| 12954 | | C * T * T * T * mU * mA * mA * mG * mC | | |
| WV- | AATACTGTCCCTTTTAAGCA | Aeo * AeoTeoAeom5Ceo * T * G * T * C * C * C | XOOOXXXXXXXXXXXXXXX | 1869 |
| 12955 | | * T * T * T * T * mA * mA * mG * mC * mA | | |
| WV- | ATACTGTCCCTTTTAAGCAA | Aeo * TeoAeom5CeoTeo * G * T * C * C * C * T | XOOOXXXXXXXXXXXXXXX | 1870 |
| 12956 | | * T * T * T * A * mA * mG * mC * mA * mA | | |
| WV- | TACTGTCCCTTTTAAGCAAC | Teo * Aeom5CeoTeoGeo * T * C * C * C * T * T | XOOOXXXXXXXXXXXXXXX | 1871 |
| 12957 | | * T * T * A * A * mG * mC * mA * mA * mC | | |
| WV- | ACTGTCCCTTTTAAGCAACC | Aeo * m5CeoTeoGeoTeo * C * C * C * T * T * T | XOOOXXXXXXXXXXXXXXX | 1872 |
| 12958 | | * T * A * A * G * mC * mA * mA * mC * mC | | |
| WV- | CTGTCCCTTTTAAGCAACCU | m5Ceo * TeoGeoTeom5Ceo * C * C * T * T * T | XOOOXXXXXXXXXXXXXXX | 1873 |
| 12959 | | * T * A * A * G * C * mA * mA * mC * mC * mU | | |
| WV- | TGTCCCTTTTAAGCAACCUA | Teo * GeoTeom5Ceom5Ceo * C * T * T * T * T | XOOOXXXXXXXXXXXXXXX | 1874 |
| 12960 | | * A * A * G * C * A * mA * mC * mC * mU * mA | | |
| WV- | GTCCCTTTTAAGCAACCUAC | Geo * Teom5Ceom5Ceom5Ceo * T * T * T * T | XOOOXXXXXXXXXXXXXXX | 1875 |
| 12961 | | * A * A * G * C * A * A * mC * mC * mU * mA * | | |
| | | mC | | |
| WV- | TCCCTTTTAAGCAACCUACA | Teo * m5Ceom5Ceom5CeoTeo * T * T * T * A | XOOOXXXXXXXXXXXXXXX | 1876 |
| 12962 | | * A * G * C * A * A * C * mC * mU * mA * mC * | | |
| | | mA | | |
| WV- | CCCTTTTAAGCAACCUACAG | m5Ceo * m5Ceom5CeoTeoTeo * T * T * A * A | XOOOXXXXXXXXXXXXXXX | 1877 |
| 12963 | | * G * C * A * A * C * C * mU * mA * mC * mA * | | |
| | | mG | | |
| WV- | CCTTTTAAGCAACCTACAGG | m5Ceo * m5CeoTeoTeoTeo * T * A * A * G * C | XOOOXXXXXXXXXXXXXXX | 1878 |
| 12964 | | * A * A * C * C * T * mA * mC * mA * mG * mG | | |
| WV- | CTTTTAAGCAACCTACAGGG | m5Ceo * TeoTeoTeoTeo * A * A * G * C * A * A | XOOOXXXXXXXXXXXXXXX | 1879 |
| 12965 | | * C * C * T * A * mC * mA * mG * mG * mG | | |
| WV- | TTTTAAGCAACCTACAGGGG | Teo * TeoTeoTeoAeo * A * G * C * A * A * C * | XOOOXXXXXXXXXXXXXXX | 1880 |
| 12966 | | C * T * A * C * mA * mG * mG * mG * mG | | |
| WV- | TTTAAGCAACCTACAGGGGC | Teo * TeoTeoAeoAeo * G * C * A * A * C * C * | XOOOXXXXXXXXXXXXXXX | 1881 |
| 12967 | | T * A * C * A * mG * mG * mG * mG * mC | | |
| WV- | TTAAGCAACCTACAGGGGCA | Teo * TeoAeoAeoGeo * C * A * A * C * C * T * | XOOOXXXXXXXXXXXXXXX | 1882 |
| 12968 | | A * C * A * G * mG * mG * mG * mC * mA | | |
| WV- | TAAGCAACCTACAGGGGCAG | Teo * AeoAeoGeom5Ceo * A * A * C * C * T * | XOOOXXXXXXXXXXXXXXX | 1883 |
| 12969 | | A * C * A * G * G * mG * mG * mC * mA * mG | | |
| WV- | AAGCAACCTACAGGGGCAGC | Aeo * AeoGeom5CeoAeo * A * C * C * T * A * | XOOOXXXXXXXXXXXXXXX | 1884 |
| 12970 | | C * A * G * G * G * mG * mC * mA * mG * mC | | |
| WV- | AGCAACCTACAGGGGCAGCC | Aeo * Geom5CeoAeoAeo * C * C * T * A * C * | XOOOXXXXXXXXXXXXXXX | 1885 |
| 12971 | | A * G * G * G * G * mC * mA * mG * mC * mC | | |
| WV- | GCAACCTACAGGGGCAGCCC | Geo * m5CeoAeoAeom5Ceo * C * T * A * C * A | XOOOXXXXXXXXXXXXXXX | 1886 |
| 12972 | | * G * G * G * G * C * mA * mG * mC * mC * | | |
| | | mC | | |
| WV- | CAACCTACAGGGGCAGCCCU | m5Ceo * AeoAeom5Ceom5Ceo * T * A * C * A | XOOOXXXXXXXXXXXXXXX | 1887 |
| 12973 | | * G * G * G * G * C * A * mG * mC * mC * mC * | | |
| | | mU | | |
| WV- | AACCTACAGGGGCAGCCCUG | Aeo * Aeom5Ceom5CeoTeo * A * C * A * G * | XOOOXXXXXXXXXXXXXXX | 1888 |
| 12974 | | G * G * G * C * A * G * mC * mC * mC * mU * | | |
| | | mG | | |
| WV- | ACCTACAGGGGCAGCCCUGG | Aeo * m5Ceom5CeoTeoAeo * C * A * G * G * | XOOOXXXXXXXXXXXXXXX | 1889 |
| 12975 | | G * G * C * A * G * C * mC * mC * mU * mG * | | |
| | | mG | | |
| WV- | AGCTTCTTGTCCAGCUUUAU | Aeo * Geom5CeoTeoTeo * C * T * T * G * T * C | XOOOXXXXXXXXXXXXXXX | 1890 |
| 12976 | | * C * A * G * C * mU * mU * mU * mA * mU | | |
| WV- | GCACUGAGAATACTGTCCCT | mG * mC * mA * mC * mU * G * A * G * A * A | XXXXXXXXXXXXXXXOOOX | 1891 |
| 12977 | | * T * A * C * T * G * Teom5Ceom5Ceom5Ceo * | | |
| | | Teo | | |
| WV- | CACUGAGAATACTGTCCCTT | mC * mA * mC * mU * mG * A * G * A * A * T * | XXXXXXXXXXXXXXXOOOX | 1892 |
| 12978 | | A * C * T * G * T * m5Ceom5Ceom5CeoTeo * | | |
| | | Teo | | |
| WV- | ACUGAGAATACTGTCCCTTT | mA * mC * mU * mG * mA * G * A * A * T * A | XXXXXXXXXXXXXXXOOOX | 1893 |
| 12979 | | * C * T * G * T * C * m5Ceom5CeoTeoTeo * | | |
| | | Teo | | |
| WV- | CUGAGAATACTGTCCCTTTT | mC * mU * mG * mA * mG * A * A * T * A * C * | XXXXXXXXXXXXXXXOOOX | 1894 |
| 12980 | | T * G * T * C * C * m5CeoTeoTeoTeo * Teo | | |
| WV- | UGAGAATACTGTCCCTTTTA | mU * mG * mA * mG * mA * A * T * A * C * T * | XXXXXXXXXXXXXXXOOOX | 1895 |
| 12981 | | G * T * C * C * C * TeoTeoTeoTeo * Aeo | | |
| WV- | GAGAATACTGTCCCTTTTAA | mG * mA * mG * mA * mA * T * A * C * T * G * | XXXXXXXXXXXXXXXOOOX | 1896 |
| 12982 | | T * C * C * C * T * TeoTeoTeoAeo * Aeo | | |
| WV- | AGAAUACTGTCCCTTTTAAG | mA * mG * mA * mA * mU * A * C * T * G * T * | XXXXXXXXXXXXXXXOOOX | 1897 |
| 12983 | | C * C * C * T * T * TeoTeoAeoAeo * Geo | | |
| WV- | GAAUACTGTCCCTTTTAAGC | mG * mA * mA * mU * mA * C * T * G * T * C * | XXXXXXXXXXXXXXXOOOX | 1898 |
| 12984 | | C * C * T * T * T * TeoAeoAeoGeo * m5Ceo | | |
| WV- | AAUACTGTCCCTTTTAAGCA | mA * mA * mU * mA * mC * T * G * T * C * C * | XXXXXXXXXXXXXXXOOOX | 1899 |
| 12985 | | C * T * T * T * T * AeoAeoGeom5Ceo * Aeo | | |
| WV- | AUACUGTCCCTTTTAAGCAA | mA * mU * mA * mC * mU * G * T * C * C * C * | XXXXXXXXXXXXXXXOOOX | 1900 |
| 12986 | | T * T * T * T * A * AeoGeom5CeoAeo * Aeo | | |
| WV- | UACUGTCCCTTTTAAGCAAC | mU * mA * mC * mU * mG * T * C * C * C * T * | XXXXXXXXXXXXXXXOOOX | 1901 |
| 12987 | | T * T * T * A * A * Geom5CeoAeoAeo * m5Ceo | | |
| WV- | ACUGUCCCTTTTAAGCAACC | mA * mC * mU * mG * mU * C * C * C * T * T * | XXXXXXXXXXXXXXXOOOX | 1902 |
| 12988 | | T * T * A * A * G * m5CeoAeoAeom5Ceo * | | |
| | | m5Ceo | | |
| WV- | CUGUCCCTTTTAAGCAACCT | mC * mU * mG * mU * mC * C * C * T * T * T * | XXXXXXXXXXXXXXXOOOX | 1903 |
| 12989 | | T * A * A * G * C * AeoAeom5Ceom5Ceo * Teo | | |
| WV- | UGUCCCTTTTAAGCAACCTA | mU * mG * mU * mC * mC * C * T * T * T * T * | XXXXXXXXXXXXXXXOOOX | 1904 |
| 12990 | | A * A * G * C * A * Aeom5Ceom5CeoTeo * Aeo | | |
| WV- | GUCCCTTTTAAGCAACCTAC | mG * mU * mC * mC * mC * T * T * T * T * A * | XXXXXXXXXXXXXXXOOOX | 1905 |
| 12991 | | A * G * C * A * A * m5Ceom5CeoTeoAeo * | | |
| | | m5Ceo | | |
| WV- | UCCCUTTTAAGCAACCTACA | mU * mC * mC * mC * mU * T * T * T * A * A * | XXXXXXXXXXXXXXXOOOX | 1906 |
| 12992 | | G * C * A * A * C * m5CeoTeoAeom5Ceo * Aeo | | |
| WV- | CCCUUTTAAGCAACCTACAG | mC * mC * mC * mU * mU * T * T * A * A * G * | XXXXXXXXXXXXXXXOOOX | 1907 |
| 12993 | | C * A * A * C * C * TeoAeom5CeoAeo * Geo | | |
| WV- | CCUUUTAAGCAACCTACAGG | mC * mC * mU * mU * mU * T * A * A * G * C | XXXXXXXXXXXXXXXOOOX | 1908 |
| 12994 | | * A * A * C * C * T * Aeom5CeoAeoGeo * Geo | | |
| WV- | CUUUUAAGCAACCTACAGGG | mC * mU * mU * mU * mU * A * A * G * C * A | XXXXXXXXXXXXXXXOOOX | 1909 |
| 12995 | | * A * C * C * T * A * m5CeoAeoGeoGeo * Geo | | |
| WV- | UUUUAAGCAACCTACAGGGG | mU * mU * mU * mU * mA * A * G * C * A * A | XXXXXXXXXXXXXXXOOOX | 1910 |
| 12996 | | * C * C * T * A * C * AeoGeoGeoGeo * Geo | | |
| WV- | UUUAAGCAACCTACAGGGGC | mU * mU * mU * mA * mA * G * C * A * A * C | XXXXXXXXXXXXXXXOOOX | 1911 |
| 12997 | | * C * T * A * C * A * GeoGeoGeoGeo * m5Ceo | | |
| WV- | UUAAGCAACCTACAGGGGCA | mU * mU * mA * mA * mG * C * A * A * C * C | XXXXXXXXXXXXXXXOOOX | 1912 |
| 12998 | | * T * A * C * A * G * GeoGeoGeom5Ceo * Aeo | | |
| WV- | UAAGCAACCTACAGGGGCAG | mU * mA * mA * mG * mC * A * A * C * C * T * | XXXXXXXXXXXXXXXOOOX | 1913 |
| 12999 | | A * C * A * G * G * GeoGeom5CeoAeo * Geo | | |
| WV- | AAGCAACCTACAGGGGCAGC | mA * mA * mG * mC * mA * A * C * C * T * A * | XXXXXXXXXXXXXXXOOOX | 1914 |
| 13000 | | C * A * G * G * G * Geom5CeoAeoGeo * | | |
| | | m5Ceo | | |
| WV- | AGCAACCTACAGGGGCAGCC | mA * mG * mC * mA * mA * C * C * T * A * C * | XXXXXXXXXXXXXXXOOOX | 1915 |
| 13001 | | A * G * G * G * G * m5CeoAeoGeom5Ceo * | | |
| | | m5Ceo | | |
| WV- | GCAACCTACAGGGGCAGCCC | mG * mC * mA * mA * mC * C * T * A * C * A * | XXXXXXXXXXXXXXXOOOX | 1916 |
| 13002 | | G * G * G * G * C * AeoGeom5Ceom5Ceo * | | |
| | | m5Ceo | | |
| WV- | CAACCTACAGGGGCAGCCCT | mC * mA * mA * mC * mC * T * A * C * A * G * | XXXXXXXXXXXXXXXOOOX | 1917 |
| 13003 | | G * G * G * C * A * Geom5Ceom5Ceom5Ceo * | | |
| | | Teo | | |
| WV- | AACCUACAGGGGCAGCCCTG | mA * mA * mC * mC * mU * A * C * A * G * G | XXXXXXXXXXXXXXXOOOX | 1918 |
| 13004 | | * G * G * C * A * G * m5Ceom5Ceom5CeoTeo | | |
| | | * Geo | | |
| WV- | ACCUACAGGGGCAGCCCTGG | mA * mC * mC * mU * mA * C * A * G * G * G | XXXXXXXXXXXXXXXOOOX | 1919 |
| 13005 | | * G * C * A * G * C * m5Ceom5CeoTeoGeo * | | |
| | | Geo | | |
| WV- | CUUGUCCAGCTTTATTGGGA | mC * mU * mU * mG * mU * C * C * A * G * C | XXXXXXXXXXXXXXXOOOX | 1920 |
| 13006 | | * T * T * T * A * T * TeoGeoGeoGeo * Aeo | | |
| WV- | AGCUUCTTGTCCAGCTTTAT | mA * mG * mC * mU * mU * C * T * T * G * T * | XXXXXXXXXXXXXXXOOOX | 1921 |
| 13007 | | C * C * A * G * C * TeoTeoTeoAeo * Teo | | |
| WV- | AUAGCAGCTTCTTGTCCAGC | mA * mU * mA * mG * mC * A * G * C * T * T * | XXXXXXXXXXXXXXXOOOX | 1922 |
| 13008 | | C * T * T * G * T * m5Ceom5CeoAeoGeo * | | |
| | | m5Ceo | | |
| WV- | CGGCCTCTGAA GCTCGGGCA | C * G * G * C * C * T * C * T * G * A * A * G * C | XXXXXXXXXX XXXXXXXXX | 1923 |
| 730 | | * T * C * G * G * G * C * A | | |
| WV- | TAGCUUCUUGU | POT * fA * mGfC * mUfU * mCfU * mUfG * | XXOXOXOXOXO | 1924 |
| 2420 | CCAGCUUUUU | mUfC * mCfA * mG * fC * mU * fU * mU * | XOXXXXXXO | |
| | | mUmU | | |
| WV- | TCACUGAGAAUAC | 5mpdT * SfC * SmAmC * SmU * SmG * | SSOSSSOSOSSSS SSSS SSSS | 1925 |
| 13570 | UGUCCCUUTU | SmAmG * SmAmA * SmU * SmA * SmC * | S | |
| | | SfU * SmG * SmU * SmC * SmC * SmC * | | |
| | | SmU * SmU * STGaNC6T * SmU | | |
| WV- | TCACUGAGAAUAC | 5mrpdT * SfC * SmAmC * SmU * SmG * | SSOSSSOSOSSSS SSSS SSSS | 1926 |
| 13571 | UGUCCCUUTU | SmAmG * SmAmA * SmU * SmA * SmC * | S | |
| | | SfU * SmG * SmU * SmC * SmC * SmC * | | |
| | | SmU * SmU * STGaNC6T * SmU | | |
| WV- | TCACUGAGAAUAC | 5mspdT * SfC * SmAmC * SmU * SmG * | SSOSSSOSOSSSS SSSS SSSS | 1927 |
| 13572 | UGUCCCUUTU | SmAmG * SmAmA * SmU * SmA * SmC * | S | |
| | | SfU * SmG * SmU * SmC * SmC * SmC * | | |
| | | SmU * SmU * STGaNC6T * SmU | | |
| WV- | TCACUGAGAAUAC | 5mpdT * SfC * SmAmC * SmU * SmG * | SSOSSSOSOSSSS SSSS SSSS | 1928 |
| 13573 | UGUCCCUUTU | SmAmG * SmAmA * SmU * SmA * SmC * | S | |
| | | SfU * SmG * SmU * SmC * SmC * SmC * | | |
| | | SmU * SmU * SAMC6T * SmU | | |
| WV- | TCACUGAGAAUAC | 5mrpdT * SfC * SmAmC * SmU * SmG * | SSOSSSOSOSSSS SSSS SSSS | 1929 |
| 13574 | UGUCCCUUTU | SmAmG * SmAmA * SmU * SmA * SmC * | S | |
| | | SfU * SmG * SmU * SmC * SmC * SmC * | | |
| | | SmU * SmU * SAMC6T * SmU | | |
| WV- | TCACUGAGAAUAC | 5mspdT * SfC * SmAmC * SmU * SmG * | SSOSSSOSOSSSS SSSS SSSS | 1930 |
| 13575 | UGUCCCUUTU | SmAmG * SmAmA * SmU * SmA * SmC * | S | |
| | | SfU * SmG * SmU * SmC * SmC * SmC * | | |
| | | SmU * SmU * SAMC6T * SmU | | |
| WV- | TCACUGAGAAUAC | 5mvpdT * SfC * SmAmC * SmU * SmG * | SSOSSSOSOSSSS SSSS SSSS | 1931 |
| 13576 | UGUCCCUUTU | SmAmG * SmAmA * SmU * SmA * SmC * | S | |
| | | SfU * SmG * SmU * SmC * SmC * SmC * | | |
| | | SmU * SmU * STGaNC6T * SmU | | |
| WV- | TCACUGAGAAUAC | 5mvpdT * SfC * SmAmC * SmU * SmG * | SSOSSSOSOSSSS SSSS SSSS | 1932 |
| 13577 | UGUCCCUUTU | SmAmG * SmAmA * SmU * SmA * SmC * | S | |
| | | SfU * SmG * SmU * SmC * SmC * SmC * | | |
| | | SmU * SmU * SAMC6T * SmU | | |
| WV- | TCACUGAGAAUAC | 5ptzdT * SfC * SmAmC * SmU * SmG * | SSOSSSOSOSSSS SSSS SSSS | 1933 |
| 13578 | UGUCCCUUTU | SmAmG * SmAmA * SmU * SmA * SmC * | S | |
| | | SfU * SmG * SmU * SmC * SmC * SmC * | | |
| | | SmU * SmU * STGaNC6T * SmU | | |
| WV- | TCACUGAGAAUAC | 5ptzdT * SfC * SmAmC * SmU * SmG * | SSOSSSOSOSSSS SSSS SSSS | 1934 |
| 13579 | UGUCCCUUTU | SmAmG * SmAmA * SmU * SmA * SmC * | S | |
| | | SfU * SmG * SmU * SmC * SmC * SmC * | | |
| | | SmU * SmU * SAMC6T * SmU | | |
| WV- | TCACUGAGAAUAC | VPT * SfC * SmAmC * SmU * SmG * | SSOSSSOSOSSSS SSSS SSS | 1935 |
| 13580 | UGUCCCTU | SmAmG * SmAmA * SmU * SmA * SmC * | | |
| | | SfU * SmG * SmU * SmC * SmC * SmC * | | |
| | | STGaNC6T * SmU | | |
| WV- | TCACUGAGAAUAC | 5mpdT * SfC * SmAmC * SmU * SmG * | SSOSSSOSOSSSS SSSS SSS | 1936 |
| 13581 | UGUCCCTU | SmAmG * SmAmA * SmU * SmA * SmC * | | |
| | | SfU * SmG * SmU * SmC * SmC * SmC * | | |
| | | STGaNC6T * SmU | | |
| WV- | TCACUGAGAAUAC | 5mrpdT * SfC * SmAmC * SmU * SmG * | SSOSSSOSOSSSS SSSS SSS | 1937 |
| 13582 | UGUCCCTU | SmAmG * SmAmA * SmU * SmA * SmC * | | |
| | | SfU * SmG * SmU * SmC * SmC * SmC * | | |
| | | STGaNC6T * SmU | | |
| WV- | TCACUGAGAAUAC | 5mspdT * SfC * SmAmC * SmU * SmG * | SSOSSSOSOSSSS SSSS SSS | 1938 |
| 13583 | UGUCCCTU | SmAmG * SmAmA * SmU * SmA * SmC * | | |
| | | SfU * SmG * SmU * SmC * SmC * SmC * | | |
| | | STGaNC6T * SmU | | |
| WV- | TCACUGAGAAUAC | VPT * SfC * SmAmC * SmU * SmG * | SSOSSSOSOSSSS SSSS SSS | 1939 |
| 13584 | UGUCCCTU | SmAmG * SmAmA * SmU * SmA * SmC * | | |
| | | SfU * SmG * SmU * SmC * SmC * SmC * | | |
| | | SAMC6T * SmU | | |
| WV- | TCACUGAGAAUAC | 5mpdT * SfC * SmAmC * SmU * SmG * | SSOSSSOSOSSSS SSSS SSS | 1940 |
| 13585 | UGUCCCTU | SmAmG * SmAmA * SmU * SmA * SmC * | | |
| | | SfU * SmG * SmU * SmC * SmC * SmC * | | |
| | | SAMC6T * SmU | | |
| WV- | TCACUGAGAAUAC | 5mrpdT * SfC * SmAmC * SmU * SmG * | SSOSSSOSOSSSS SSSS SSS | 1941 |
| 13586 | UGUCCCTU | SmAmG * SmAmA * SmU * SmA * SmC * | | |
| | | SfU * SmG * SmU * SmC * SmC * SmC * | | |
| | | SAMC6T * SmU | | |
| WV- | TCACUGAGAAU | 5mspdT * SfC * SmAmC * SmU * SmG * | SSOSSSOSO SSSS SSSS SSS | 1942 |
| 13587 | ACUGUCCCTU | SmAmG * SmAmA * SmU * SmA * SmC * SfU * | | |
| | | SmG * SmU * SmC * SmC * SmC * SAMC6T * | | |
| | | SmU | | |

The disclosure notes that some sequences, due to their length, are divided into multiple lines; however, these sequences, as are all oligonucleotides in Table 1A, are single-stranded (unless otherwise noted).
Moieties and Modifications Listed in the Tables (or Compounds Used to Construct Oligonucleotides Comprising these Moieties or Modifications:

| | |
|---|---|
| IT | 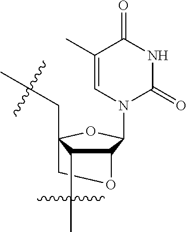 [SMILES: *C[C@]12CO[C@@H](C1*)[C@H](n1cc(C)c(=O)[nH]c1=O)O2] if between 5′-end groups and/or internucleotidic linkages (e.g., in WV-3819); |
| | 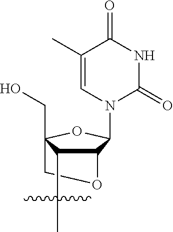 [SMILES: *C1[C@@H]2OC[C@]1(CO)O[C@H]2n1cc(C)c(=O)[nH]c1=O] if at 5′-end and without 5′-end groups (e.g., in WV-3818); |
| | 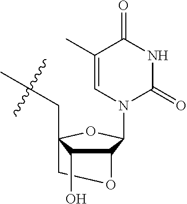 [SMILES: *C[C@]12CO[C@@H](C1O)[C@H](n1cc(C)c(=O)[nH]c1=O)O2] if at 3′-end (e.g., in WV-7821). |
| IG | 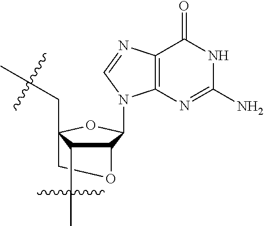 [SMILES: *C[C@]12CO[C@@H](C1*)[C@H](n1cnc3c(=O)[nH]c(N)nc31)O2] if between 5′-end groups and/or internucleotidic linkages (e.g., in WV-6689); |
| | 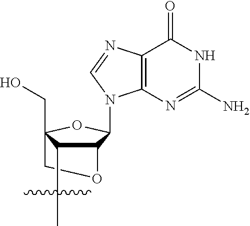 [SMILES: *C1[C@@H]2OC[C@]1(CO)O[C@H]2n1cnc2c(=O)[nH]c(N)nc21] if at 5′-end and without 5′-end groups (e.g., in WV-6711); |
| | 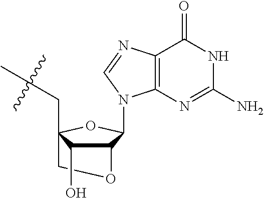 [SMILES: *C[C@]12CO[C@@H](C1O)[C@H](n1cnc3c(=O)[nH]c(N)nc31)O2] if at 3′-end (e.g., in WV-7827). |
| IA | 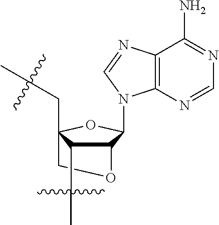 [SMILES: *C[C@]12CO[C@@H](C1*)[C@H](n1cnc3c(N)ncnc31)O2] if between 5′-end groups and/or internucleotidic linkages (e.g., in WV-6692); |
| | 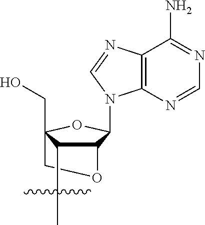 [SMILES: *C1[C@@H]2OC[C@]1(CO)O[C@H]2n1cnc2c(N)ncnc21] if at 5′-end and without 5′-end groups (e.g., in WV-6710); |
| | 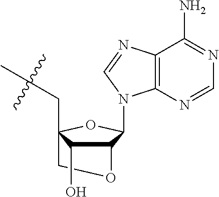 [SMILES: *C[C@]12CO[C@@H](C1O)[C@H](n1cnc3c(N)ncnc31)O2] if at 3′-end (e.g., in WV-7817). |
| Im5C | 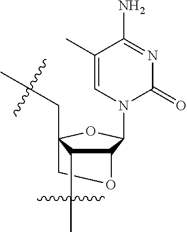 [SMILES: *C[C@]12CO[C@@H](C1*)[C@H](n1cc(C)c(N)nc1=O)O2] if between 5′-end groups and/or intemucleotidic linkages (e.g., in WV-6690); |
| | 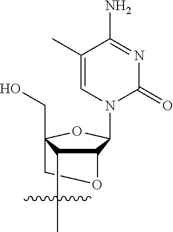 [SMILES: *C1[C@@H]2OC[C@]1(CO)O[C@H]2n1cc(C)c(N)nc1=O] if at 5′-end and without 5′-end groups (e.g., in WV-7817); |
| | 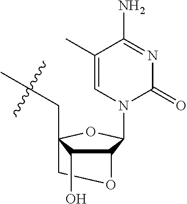 [SMILES: *C[C@]12CO[C@@H](C1O)[C@H](n1cc(C)c(N)nc1=O)O2] if at 3′-end (e.g., in WV-7818). |
| MeOT | 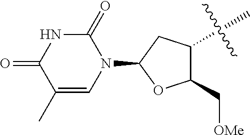 |
| Mod001 | 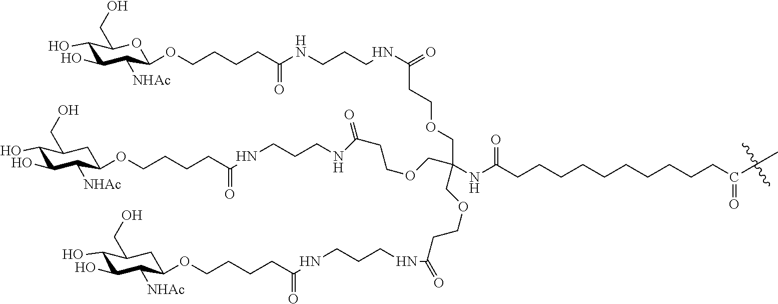 |
| Mod022 | CH₃CH₂CH₂—; connected to 5′-end of oligonucleotide chain through either a phosphate |
| | linkage (O or PO) or phosphorothioate linkage (* if the phosphorothioate not chirally |
| | controlled; can also be Sp if chirally controlled and has an Sp configuration, and Rp if |
| | chirally controlled and has an Rp configuration) as illustrated. |
| | 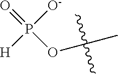 [SMILES: *O[PH](=O)[O-]] |
| Mod023 | 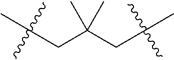 [SMILES: *CC(C)(C)C*] connected to 5′-end of oligonucleotide chain through either a phosphate linkage (O or PO) or phosphorothioate linkage (* if the phosphorothioate not chirally controlled; can also be Sp if chirally controlled and has an Sp configuration, and Rp if chirally controlled and has an Rp configuration) as illustrated. |
| | 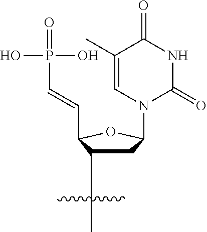 |
| Mod034 | 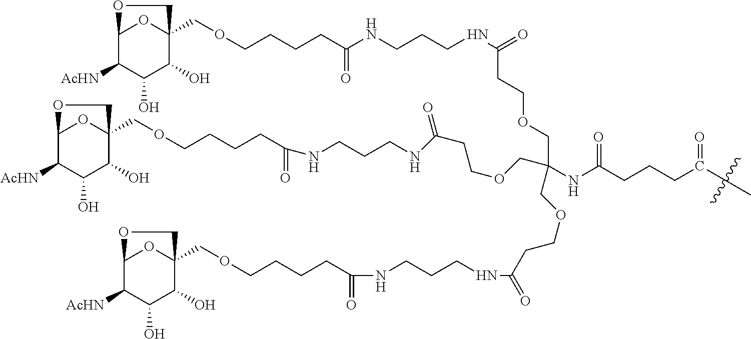 [SMILES: *C(=O)CCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| Mod035 | 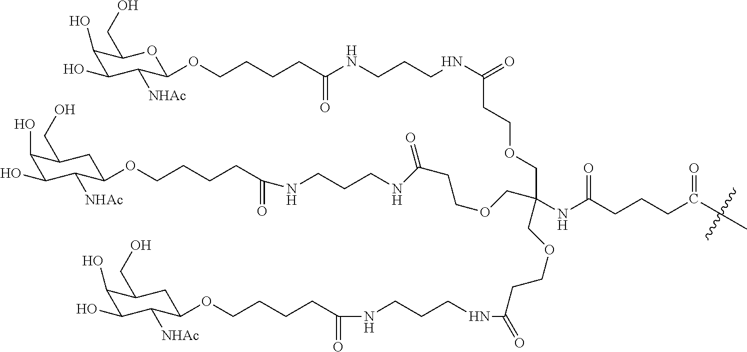 [SMILES: *C(=O)CCCC(=O)NC(COCCC(=O)NCCCNC(=O)CCCCOC1CC(CO)C(O)C(O)C1NC(C)=O)(COCCC(=O)NCCCNC(=O)CCCCOC1CC(CO)C(O)C(O)C1NC(C)=O)COCCC(=O)NCCCNC(=O)CCCCOC1OC(CO)C(O)C(O)C1NC(C)=O] |
| Mod036 | 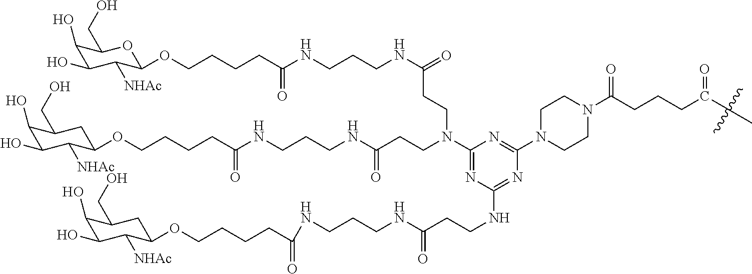 [SMILES: *C(=O)CCCC(=O)N1CCN(c2nc(NCCC(=O)NCCCNC(=O)CCCCOC3CC(CO)C(O)C(O)C3NC(C)=O)nc(N(CCC(=O)NCCCNC(=O)CCCCOC3CC(CO)C(O)C(O)C3NC(C)=O)CCC(=O)NCCCNC(=O)CCCCOC3OC(CO)C(O)C(O)C3NC(C)=O)n2)CC1] |
| Mod038 | 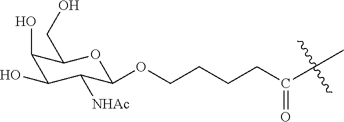 [SMILES: *C(=O)CCCCOC1OC(CO)C(O)C(O)C1NC(C)=O] |
| Mod039 | 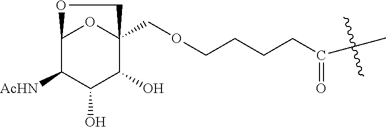 [SMILES: *C(=O)CCCCOC[C@@]12CO[C@@H](O1)[C@H](NC(C)=O)[C@@H](O)[C@H]2O] |
| Mod040 | 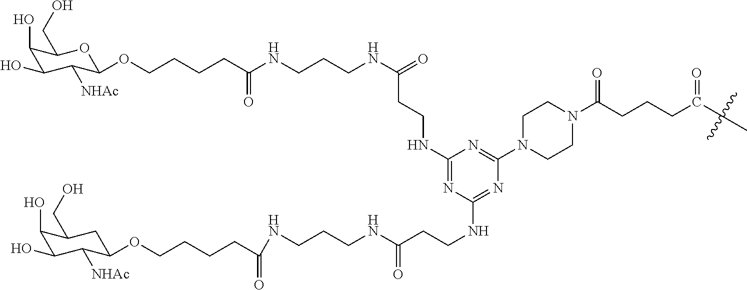 [SMILES: *C(=O)CCCC(=O)N1CCN(c2nc(NCCC(=O)NCCCNC(=O)CCCCOC3CC(CO)C(O)C(O)C3NC(C)=O)nc(NCCC(=O)NCCCNC(=O)CCCCOC3OC(CO)C(O)C(O)C3NC(C)=O)n2)CC1] |
| Mod041 | 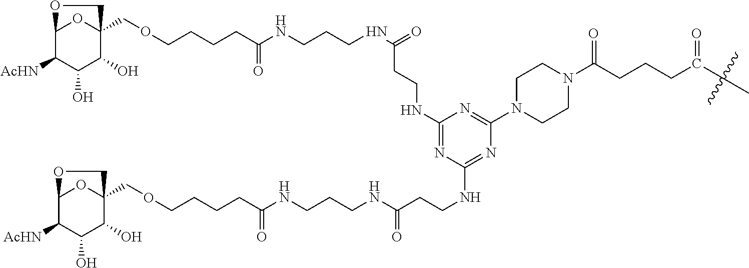 [SMILES: *C(=O)CCCC(=O)N1CCN(c2nc(NCCC(=O)NCCCNC(=O)CCCCOC[C@@]34CO[C@@H](O3)[C@H](NC(C)=O)[C@@H](O)[C@H]4O)nc(NCCC(=O)NCCCNC(=O)CCCCOC[C@@]34CO[C@@H](O3)[C@H](NC(C)=O)[C@@H](O)[C@H]4O)n2)CC1] |
| Mod079 | 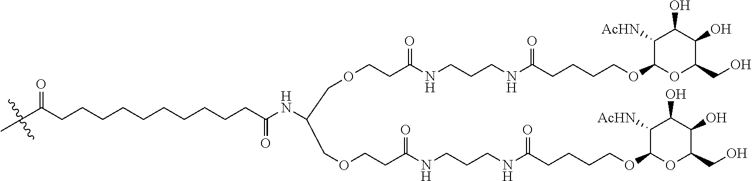 [SMILES: *C(=O)CCCCCCCCCCC(=O)NC(COCCC(=O)NCCCNC(=O)CCCCO[C@@H]1O[C@H](CO)[C@H](O)[C@H](O)[C@H]1NC(C)=O)COCCC(=O)NCCCNC(=O)CCCCO[C@@H]1O[C@H](CO)[C@H](O)[C@H](O)[C@H]1NC(C)=O] |
| Mod080 | 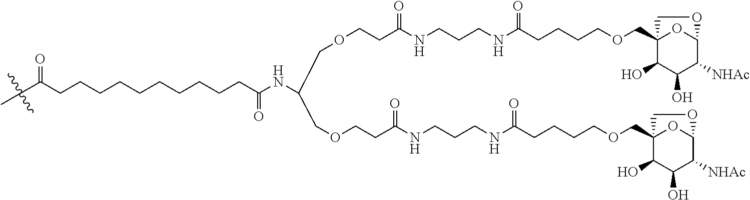 [SMILES: *C(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| Mod081 | 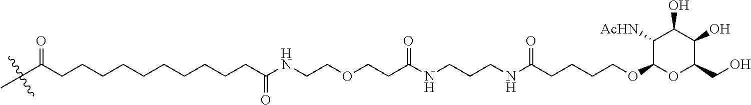 [SMILES: *C(=O)CCCCCCCCCCC(=O)NCCOCCC(=O)NCCCNC(=O)CCCCO[C@@H]1O[C@H](CO)[C@H](O)[C@H](O)[C@H]1NC(C)=O] |
| Mod082 | 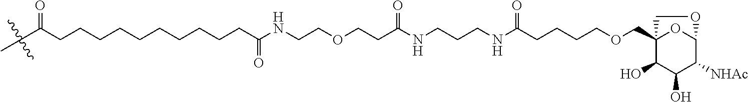 [SMILES: *C(=O)CCCCCCCCCCC(=O)NCCOCCC(=O)NCCCNC(=O)CCCCOC[C@@]12CO[C@@H](O1)[C@H](NC(C)=O)[C@@H](O)[C@H]2O] |
| Mod083 | 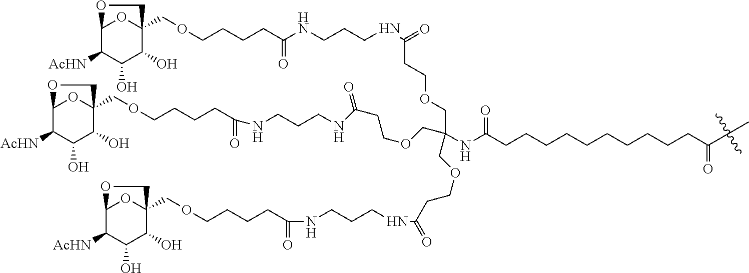 [SMILES: *C(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| 5mp | 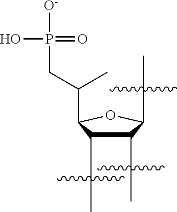 |
| 5MR | 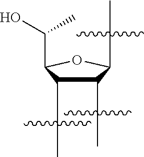 |
| 5mrp | 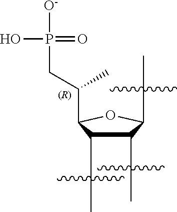 |
| 5MS | 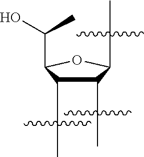 |
| 5msp | 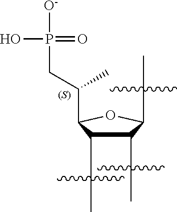 |
| 5mvp | 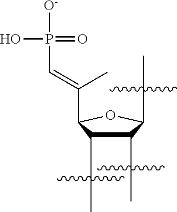 |
| 5pacet | 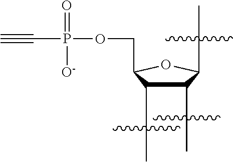 |
| 5ptz | 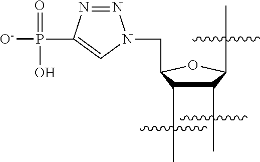 |
| 5tz | 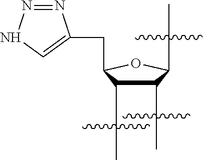 |
| 5tzpo | 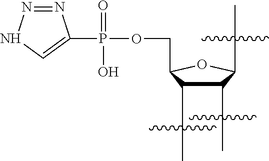 |
| 5mpdT | 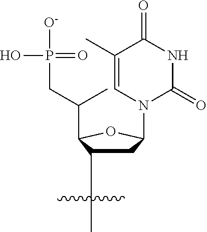 |
| 5MRdT | 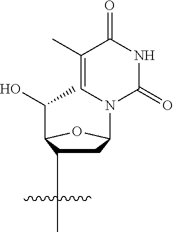 |
| 5mrpdT | 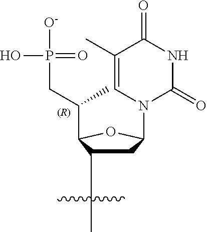 |
| 5MSdT | 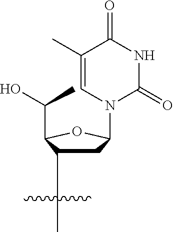 |
| 5mspdT | 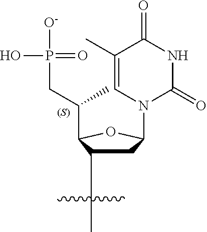 |
| 5mvpdT | 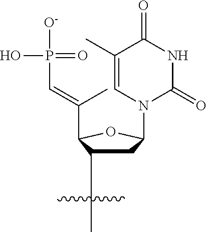 |
| 5pacetdT | 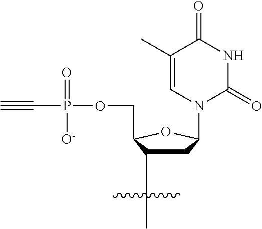 |
| 5ptzdT | 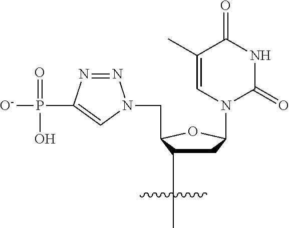 |
| 5tzdT | 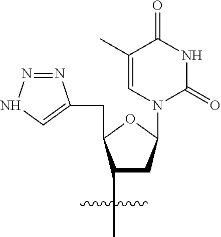 [SMILES: *[C@@H]1C[C@H](n2cc(C)c(=O)[nH]c2=O)O[C@@H]1Cc1c[nH]nn1] |
| 5tzpodT | 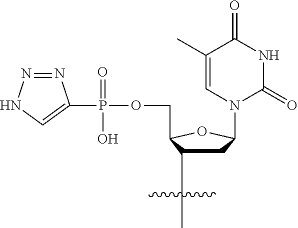 |
| tbclc6T | 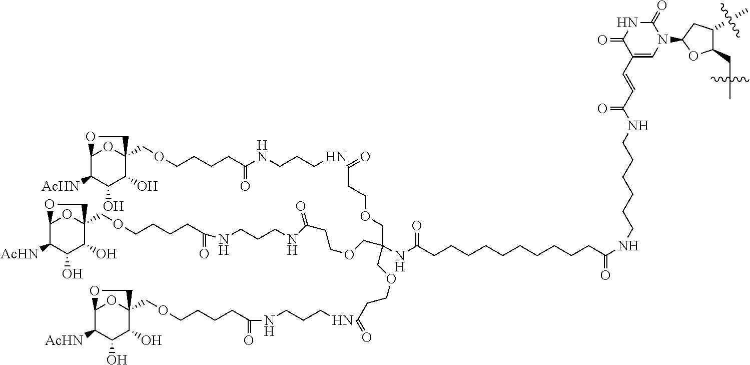 |
| bbclc6T | 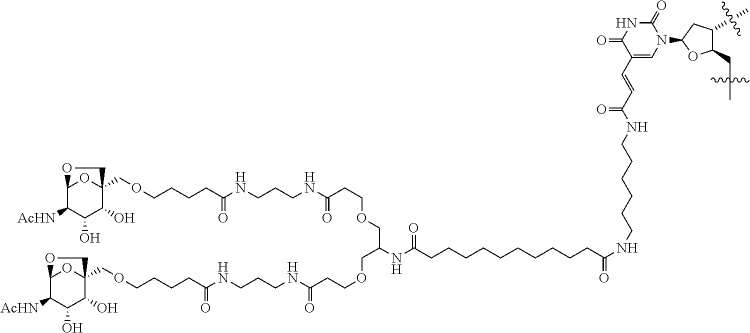 |
| L009 | —CH₂CH₂CH₂—. When L009 is present at the 5′-end of an oligonucleotide |
| | without a Mod, one end of L009 is connected to —OH and the other end connected |
| | to a 5′-carbon of the oligonucleotide chain via an linkage as indicated (e.g., in |
| | WV-9261, via a stereorandom phosphorothioate linkage (“*”)). |

In some embodiments, a linker, e.g., L009, L010, etc., can replace a sugar, and is bonded on either end to an internucleotidic linkage. For example:
WV-9266 comprises . . . * mAL009*mUfG* . . . , which represents, from 5′ to 3′, a phosphorothioate (*), a sugar which is 2′-OMe (m) attached to a base (A), a phosphodiester linkage (not indicated), a L009 linker (L009), a phosphorothioate (*), a sugar which is 2′-OMe attached to a base which is U (mU), a phosphodiester linkage (not indicated), a sugar which is 2′-F (f) attached to a base (G) and a phosphorothioate.
WV-9267 comprises . . . * mAfC * L009fG* . . . , which represents, from 5′ to 3′, a phosphorothioate (*), a sugar which is 2′-OMe (m) attached to a base (A), a phosphodiester (not indicated), a sugar which is 2′-F (f) attached to a base (C), a phosphorothioate, a L009 linker (L009), a phosphodiester linkage (not indicated), a sugar which is 2′-F (f) attached to a base (G), and a phosphorothioate.

| | |
|---|---|
| L010 | 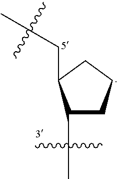 L010 is connected in the same fashion as typically in DNA (the 5′-carbon of a first sugar is connected to a 3′-carbon of a second sugar via an internucleotidic linkage, and the 3′- carbon of the first sugar is connected to the 5′-carbon of a third sugar via an internucleotidic linkage). When L010 is present at the 5′-end of an oligonucleotide without a Mod, the 5′-carbon of L010 is connected to —OH and the 3'-carbon connected to a 5'-carbon of the oligonucleotide chain via a linkage as indicated (e.g., in WV-9250, via a stereorandom phosphorothioate linkage (“*”)). |

In some oligonucleotides, L010 replaces a sugar bound to a base, and L010 is bond on either end to an internucleotidic linkage.

| | |
|---|---|
| n001, nX | |
| VPT | |
| VP | 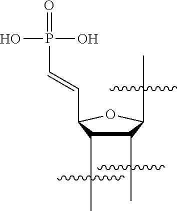 |
| VQ | 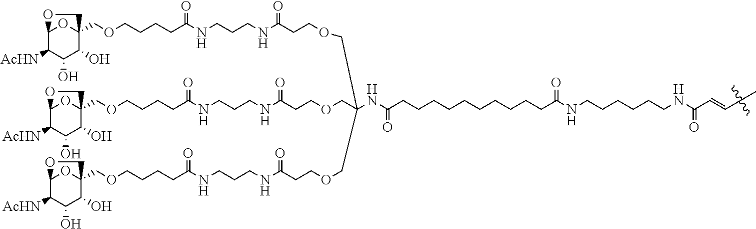 [SMILES: */C=C/C(=O)NCCCCCCNC(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| VR | 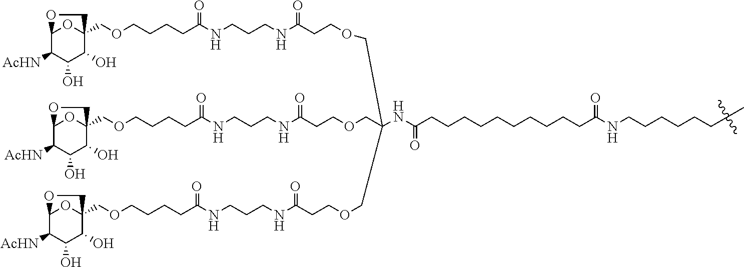 [SMILES: *CCCCCCNC(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| VS | 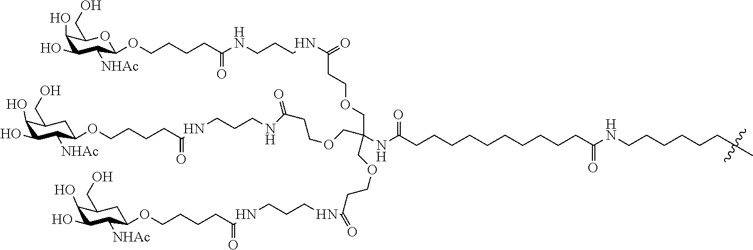 |
| VT | 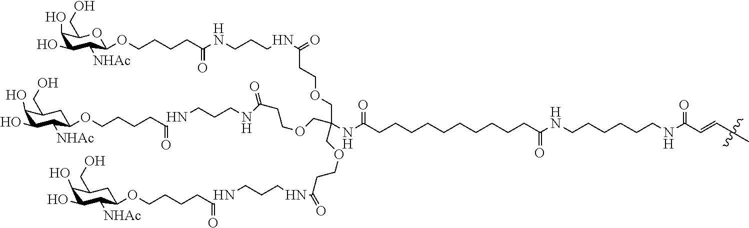 |

### Additional Abbreviations

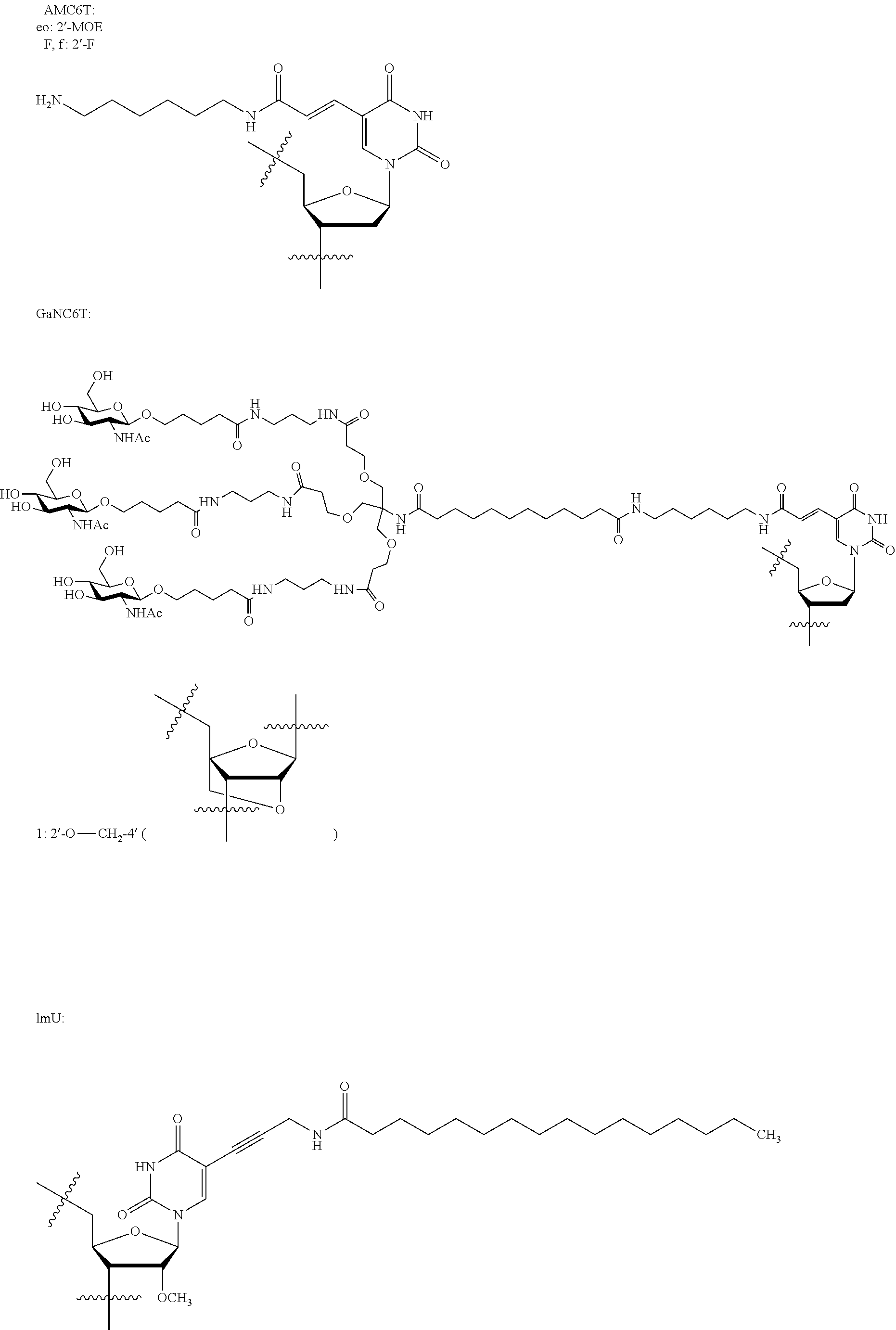

L001: —NH—(CH₂)₆-linker (C6 linker, C6 amine linker or C6 amino linker), connected to Mod, if any (if no Mod, —H, e.g., in WV-8240), through —NH—, and the 5′-end (e.g., in WV-2406) or 3′-end of oligonucleotide chain through either a phosphate linkage (0 or PO) or phosphorothioate linkage (* if the phosphorothioate not chirally controlled; can also be Sp if chirally controlled and has an Sp configuration, and Rp if chirally controlled and has an Rp configuration) as illustrated. For example, in WV-2406, L001 is connected to Mod001 through —NH— (forming an amide group —C(O)—NH—), and is connected to the oligonucleotide chain through a phosphate linkage (OXXXXXXXXXXXXXXXXXXX); in WV-2422, L001 is not connected to any Mod, but —H, through —NH—, and is connected to the oligonucleotide chain through a phosphate linkage (OXXXXXXXXXXXXXXXXXXX)

### L003:

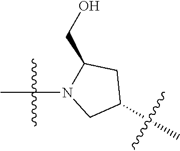

linker, connected to Mod, if any (if no Mod, —H, e.g., in WV-2426), through its amino group, and the 5′-end (e.g., in WV-2407) or 3′-end (e.g., in WV-8070) of oligonucleotide chain through either a phosphate linkage (0 or PO) or phosphorothioate linkage (* if the phosphorothioate not chirally controlled; can also be Sp if chirally controlled and has an Sp configuration, and Rp if chirally controlled and has an Rp configuration) as illustrated. For example, in WV-2407, L003 is connected to Mod001 through its amino group (forming an amide group

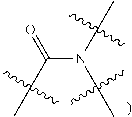 [SMILES: *C(=O)N(*)*]

and is connected to the 5′-end of oligonucleotide chain through a phosphate linkage (OXXXXXXXXXXXXXXXXXXX); in WV-2426, L001 is not connected to any Mod, but —H, through —NH—, and is connected to the oligonucleotide chain through a phosphate linkage (OXXXXXXXXXXXXXXXXXXX); in WV-8070, L003 is connected to Mod001 through its amino group (forming an amide group

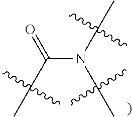 [SMILES: *C(=O)N(*)*]

and is connected to the 3′-end of oligonucleotide chain through a phosphate linkage ( . . . XXXXXXXXXXXXXXXXXXXO)

### m: 2′-OMe

m5: methyl at 5-position of C (nucleobase is 5-methylcytosine)
m5Ceo: 5-methyl 2′-methoxyethyl C

### OMe: 2′-OMe

O, PO: phoshodiester (phosphate); can be an end group (typically “PO”; for example in WV-4260: POT*fC* . . . ), or a linkage, e.g., a linkage
between linker and oligonucleotide chain, an internucleotidic linkage, etc.
*, PS: Phosphorothioate; can be an end group (typically “PS”, for example, in WV-2653: PST*fA* . . . ), or a linkage, e.g., a linkage between
linker and oligonucleotide chain, an internucleotidic linkage, etc.
R, Rp: Phosphorothioate in Rp conformation
S, Sp: Phosphorothioate in Sp conformation
X: Stereorandom phosphorothioate

In some embodiments, a provided oligonucleotide composition is a single-stranded RNAi agent listed in Table 1A or otherwise described herein. In some embodiments, example properties of provided oligonucleotides were demonstrated.

In some embodiments, a provided oligonucleotide has a structure of any of formats illustrated in FIG. 1.

The present disclosure presents many non-limiting examples of oligonucleotides capable of mediating single-stranded RNA interference (e.g., single-stranded RNAi agents). Experimental data (not shown) demonstrated that various putative single-stranded RNAi agents were, in fact, capable of mediating RNA interference. In some experiments, an in vitro Ago-2 assay was used, including the use of a RNA test substrate WV-2372 (APOC3). The band representing the RNA test substrate is absent in the presence of oligonucleotides WV-1308 and WV-2420, indicating that these oligonucleotides are single-stranded RNAi agents capable of mediating RNA interference. The remaining lanes are controls: Substrate in the absence of negative control ASO WV-2134; substrate in the presence of negative control ASO WV-2134, which does not mediate RNA interference; substrate in the absence of test oligonucleotide WV-1308; substrate in the absence of test oligonucleotide WV-2420; substrate alone; no substrate, with added WV-2134; and no substrate, with added WV-1308. Also performed (data not shown) was an in vitro Ago-2 assay, using an APOC3 mRNA as a test substrate in a 3′ RACE assay in Hep3B cells. A cleavage product of the APOC3 mRNA in the presence of test oligonucleotide WV-3021 was detected, the product corresponding to cleavage of the mRNA at a site corresponding to a cut between positions 10 and 11 of WV-3021. An artifactual cleavage product was also detected. Experimental data (not shown) demonstrated that dual mechanism oligonucleotide WV-2111 is capable of mediating knockdown by both RNase H and RNA interference. In an experiment, several oligonucleotides were capable of mediating RNA interference. The RNA test substrate was WV-2372. The experiment showed disappearance of the RNA test substrate in the presence of test oligonucleotides WV-1308; WV-2114; WV-2386; and WV-2387, indicating that all these oligonucleotides are capable of acting as single-stranded RNAi agents mediating RNA interference. The remaining lanes are controls. Thus, the experiment showed that oligonucleotides WV-1308, WV-2114, WV-2386, and WV-2387 were all able to mediate RNA interference. Thus, the experiments showed that several single-stranded RNAi agents (e.g., WV-1308, WV-2420, WV-3021, WV-2111, WV-2114, WV-2386, and WV-2387) are capable of mediating RNA interference. The present disclosure presents many non-limiting examples of oligonucleotides, having any of various sequences, formats, modifications, 5′-end regions, seed regions, post-seed regions, and 3′-end regions, and which are capable of mediating single-stranded RNA interference (e.g., single-stranded RNAi agents).

Formats of Oligonucleotides

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides can have any format or portion thereof or structural element thereof described herein or known in the art.

In some embodiments, an APOC3 oligonucleotide can have any format or structural element thereof described herein or known in the art.

In some embodiments, an APOC3 oligonucleotide capable of directing a decrease in the expression and/or level of a target gene or its gene product can have any format or structural element thereof described herein or known in the art.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can have any format or structural element thereof described herein or known in the art.

Additional non-limiting examples of various ssRNAi formats are embodied by various single-stranded RNAi agents described herein.

In some embodiments, a provided single-stranded RNAi comprises a 5′-end represented by any 5′-end of any single-stranded RNAi format depicted in FIG. 1 or any single-stranded RNAi agent or single-stranded RNAi format described herein.

In some embodiments, a provided single-stranded RNAi comprises a 5′-end structure or 5′-end region represented by any 5′-end structure or 5′-end region of any single-stranded RNAi format depicted in FIG. 1 or any single-stranded RNAi agent or single-stranded RNAi format described herein.

In some embodiments, a provided single-stranded RNAi comprises a 5′-nucleotide represented by any 5′-nucleotide of any single-stranded RNAi format depicted in FIG. 1 or any single-stranded RNAi agent or single-stranded RNAi format described herein.

In some embodiments, a provided single-stranded RNAi comprises a 5′-nucleoside represented by any 5′-nucleoside of any single-stranded RNAi format depicted in FIG. 1 or any single-stranded RNAi agent or single-stranded RNAi format described herein.

In some embodiments, a provided single-stranded RNAi comprises a seed region represented by any seed region of any single-stranded RNAi format depicted in FIG. 1 or any single-stranded RNAi agent or single-stranded RNAi format described herein.

In some embodiments, a provided single-stranded RNAi comprises a post-seed region represented by any post-seed region of any single-stranded RNAi format depicted in FIG. 1 or any single-stranded RNAi agent or single-stranded RNAi format described herein.

In some embodiments, a provided single-stranded RNAi comprises a post-seed region or component thereof represented by any post-seed region or component thereof of any single-stranded RNAi format depicted in FIG. 1 or any single-stranded RNAi agent or single-stranded RNAi format described herein.

In some embodiments, a provided single-stranded RNAi comprises a 3′-terminal dinucleotide represented by any 3′-terminal dinucleotide of any single-stranded RNAi format depicted in FIG. 1 or any single-stranded RNAi agent or single-stranded RNAi format described herein.

In some embodiments, a provided single-stranded RNAi comprises a seed region having a pattern of internucleotidic linkages represented by the pattern of internucleotidic linkages of any seed region of any single-stranded RNAi format depicted in FIG. 1 or any single-stranded RNAi agent or single-stranded RNAi format described herein.

In some embodiments, a provided single-stranded RNAi comprises a post-seed region having a pattern of internucleotidic linkages represented by the pattern of internucleotidic linkages of any post-seed region of any single-stranded RNAi format depicted in FIG. 1 or any single-stranded RNAi agent or single-stranded RNAi format described herein.

In some embodiments, a provided single-stranded RNAi comprises a post-seed region or component thereof having a pattern of internucleotidic linkages represented by the pattern of internucleotidic linkages of any post-seed region or component thereof of any single-stranded RNAi format depicted in FIG. 1 or any single-stranded RNAi agent or single-stranded RNAi format described herein.

In some embodiments, a provided single-stranded RNAi comprises a 3′-terminal dinucleotide having a pattern of internucleotidic linkages represented by the pattern of internucleotidic linkages of any 3′-terminal dinucleotide of any single-stranded RNAi format depicted in FIG. 1 or any single-stranded RNAi agent or single-stranded RNAi format described herein.

In some embodiments, a provided single-stranded RNAi comprises a seed region having a pattern of chemical modifications represented by the pattern of chemical modifications of any seed region of any single-stranded RNAi format depicted in FIG. 1 or any single-stranded RNAi agent or single-stranded RNAi format described herein.

In some embodiments, a provided single-stranded RNAi comprises a post-seed region having a pattern of chemical modifications represented by the pattern of chemical modifications of any post-seed region of any single-stranded RNAi format depicted in FIG. 1 or any single-stranded RNAi agent or single-stranded RNAi format described herein.

In some embodiments, a provided single-stranded RNAi comprises a post-seed region or component thereof having a pattern of chemical modifications represented by the pattern of chemical modifications of any post-seed region or component thereof of any single-stranded RNAi format depicted in FIG. 1 or any single-stranded RNAi agent or single-stranded RNAi format described herein.

In some embodiments, a provided single-stranded RNAi comprises a 3′-terminal dinucleotide having a pattern of chemical modifications represented by the pattern of chemical modifications of any 3′-terminal dinucleotide of any single-stranded RNAi format depicted in FIG. 1 or any single-stranded RNAi agent or single-stranded RNAi format described herein.

In some embodiments, a provided single-stranded RNAi comprises a chemical modification represented by any chemical modification of any single-stranded RNAi format depicted in FIG. 1 or any single-stranded RNAi agent or single-stranded RNAi format described herein.

In some embodiments, a provided single-stranded RNAi comprises a chemical modification represented by any chemical modification of any single-stranded RNAi format depicted in

FIG. 1 or described herein, wherein the chemical modification is conjugation of a moiety comprising a phosphate, linker, or a targeting moiety.

In some embodiments, a provided single-stranded RNAi comprises a chemical modification represented by any chemical modification of any single-stranded RNAi format depicted in FIG. 1 or described herein, wherein the chemical modification is conjugation of a moiety comprising a phosphate, linker, or a targeting moiety, wherein the targeting moiety comprises a GalNAc moiety. In some embodiments, a GalNAc is a protected or de-protected GalNAc.

In some embodiments, an APOC3 oligonucleotide is capable of decreasing the expression, activity and/or level of a target gene and/or a gene product thereof and has the format of any oligonucleotide described herein. In some embodiments, an APOC3 oligonucleotide is capable of decreasing the expression, activity and/or level of a target gene and/or a gene product thereof via a RNaseH-mediated mechanism or mechanism related to steric hindrance of translation and has the format of any oligonucleotide described herein. In some embodiments, an APOC3 oligonucleotide is capable of decreasing the expression, activity and/or level of a target gene and/or a gene product thereof via a RNaseH-mediated mechanism or mechanism related to steric hindrance of translation and has an asymmetric format. In some embodiments, an APOC3 oligonucleotide which has an asymmetric format comprises a first wing, a core and a second wing, wherein the core comprises a region of 5 or more contiguous 2′-deoxy nucleotides which can anneal to a target mRNA and form a structure recognized by RNaseH, and wherein the structure of the first and second wings are different. In some embodiments, the first and second wings differ in their 2′-modifications and/or internucleotidic linkages, or pattern of stereochemistry of the internucleotidic linkages.

In some embodiments, an APOC3 oligonucleotide is capable of decreasing the expression, activity and/or level of a target gene and/or a gene product thereof comprises a neutral internucleotidic linkage (e.g., a neutral backbone).

In some embodiments, an APOC3 oligonucleotide comprises a neutral backbone. In some embodiments, an APOC3 oligonucleotide comprises an internucleotidic linkage which is or comprises a triazole, neutral triazole, or alkyne. In some embodiments, a nucleic acid (including but not limited to an APOC3 oligonucleotide) which comprises an internucleotidic linkage which comprises a triazole, neutral triazole, or alkyne, wherein the internucleotidic linkage is stereocontrolled and in the Rp or Sp configuration. In some embodiments, an internucleotidic linkage comprising a triazole has a formula of:

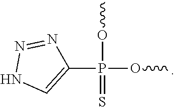

In some embodiments, an internucleotidic linkage comprising a neutral triazole has the formula of:

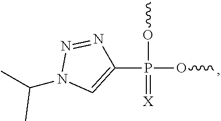

where X is O or S. In some embodiments, an internucleotidic linkage comprising an alkyne has the formula of:

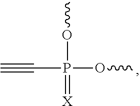

wherein X is O or S.
In some embodiments, an internucleotidic linkage comprises a cyclic guanidine. In some embodiments, an internucleotidic linkage comprises a cyclic guanidine and has the structure of:

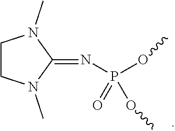

In some embodiments, a neutral internucleotidic linkage or internucleotidic linkage comprising a cyclic guanidine is stereochemically controlled. In some embodiments, a neutral internucleotidic linkage improves the activity, delivery and/or stability of an APOC3 oligonucleotide and/or the ability of an APOC3 oligonucleotide to perform endosomal escape.

As appreciated by those skilled in the art, in some instances, [symbol] may be used to indicate a connection site (as

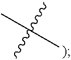

in some instances, [symbol] may be used to indicate a stereorandom connection.

Length of an APOC3 Oligonucleotide

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides can have any length, wherein the length of an APOC3 oligonucleotide is such that the oligonucleotide is capable of directing a decrease in the expression and/or level of a target gene or its gene product.

In some embodiments, the length of an APOC3 oligonucleotide is such that the oligonucleotide is capable of directing a decrease in the expression and/or level of a target gene or its gene product.

In some embodiments, the length of a RNAi agent is such that the RNAi agent is capable of directing RNA interference of a specific target transcript in a sequence-specific manner. In some embodiments, the RNAi agent comprises a sufficient number of nucleobases of sufficient identity to recognize a target transcript. In some embodiments, the RNAi agent is also be of a length suitable for mediating RNAi interference.

The target portion of the sequence will be at least long enough to serve as a substrate for iRNA-directed cleavage at or near that portion. For example, the target sequence will generally be from about 9-36 nucleotides (“nt”) in length, e.g., about 15-30 nucleotides in length, including all sub-ranges therebetween. Examples of single-stranded RNAi agents of various lengths are shown in Table 1A.

FIG. 1 illustrates non-limiting examples of single-stranded RNAi agents having lengths from 19 to 25. Single-stranded RNAi agents having any of each of these lengths were constructed and found to be capable of knocking down a target gene. Thus, a provided single-stranded RNAi agent can be any of a variety of different lengths.

Non-limiting examples of formats of ssRNAi agents which are 19 bases long include: Formats 20-21 of FIG. 1.

Non-limiting examples of formats of ssRNAi agents which are 20 bases long include: Format 19 of FIG. 1.

5′-End of an APOC3 Oligonucleotide, Including a Single-Stranded RNAi Agent

In some embodiments, the structure of the 5′-end of an APOC3 oligonucleotide is such that the oligonucleotide is capable of directing a decrease in the expression and/or level of a target gene or its gene product.

In some embodiments, the structure of the 5′-end of a RNAi agent is such that the RNAi agent is capable of directing RNA interference of a specific target transcript in a sequence-specific manner.

In some embodiments, a provided oligonucleotide can comprise any 5′-end region, 5′-end structure, 5′-end group, 5′-end nucleoside, or 5′-end nucleotide described herein or known in the art. In some embodiments, a provided oligonucleotide capable of directing RNase H-mediated knockdown can comprise any 5′-end region, 5′-end structure, 5′-end group, 5′-end nucleoside, or 5′-end nucleotide described herein or known in the art. In some embodiments, a provided oligonucleotide capable of directing RNA interference can comprise any 5′-end region, 5′-end structure, 5′-end group, 5′-end nucleoside, or 5′-end nucleotide described herein or known in the art. In some embodiments, a provided oligonucleotide capable of directing RNA interference and RNase H-mediated knockdown can comprise any 5′-end region, 5′-end structure, 5′-end group, 5′-end nucleoside, or 5′-end nucleotide described herein or known in the art.

Among other things, the present disclosure recognizes that 5′-end structures of oligonucleotides, optionally in combination with additional features in accordance with the present disclosure, can provide unexpected advantages. In some embodiments, the present disclosures provides 5′-end groups (corresponding to 5′-HO—CH₂— of ribose found in natural RNA (or deoxyribose found in natural DNA)) that can surprisingly improve one or more properties and/or activities (e.g., stability, activity, manufacture cost, etc.) of oligonucleotides.

In some embodiments, 5′—OH groups of provided oligonucleotides are unmodified, i.e., they exist as free —OH. In some embodiments, a 5′-end group is 5′-HO—CH₂—. Among other things, the present disclosure demonstrates that a provided oligonucleotide with free 5′—OH groups can achieve properties and/or activities (e.g., stability, RNAi activity when used as ss-RNAi agent, etc.) comparable to an otherwise identical oligonucleotide comprising 5′-phosphate (or derivatives thereof) groups, despite reports in the literature that certain activities, e.g., RNAi activity, require presence of 5′-phosphate groups.

In some embodiments, a 5′-end group comprises no phosphorus atom. In some embodiments, a 5′-end group comprises no phosphate groups, or derivatives or bioisosteres thereof. In some embodiments, a 5′-end group comprises no acidic groups. In some embodiments, a 5′-end group comprises no carboxyl groups. In some embodiments, a 5′-end comprises no phosphorus atom or carboxyl groups. In some embodiments, a 5′-end group is 5′-HO—CH₂—. Among other things, the present disclosure demonstrates that provided oligonucleotides with no 5′-phosphates or derivatives or bioisosteres thereof can surprisingly achieve activities comparable to oligonucleotides that have 5′-phosphates but are otherwise identical, for example, in knock-down of mRNA levels of target genes, through RNAi pathways.

In some embodiments, a 5′-nucleoside unit of a provided oligonucleotide (which includes the sugar and nucleobase moieties but not the internucleotidic linkage between the 5′-nucleoside unit and the second nucleoside unit from the 5′-end) comprises no phosphate group, or derivatives or bioisosteres thereof. In some embodiments, a 5′-nucleoside unit comprises no phosphorus atom. In some embodiments, a 5′-nucleoside comprises no acidic groups. In some embodiments, a 5′-nucleoside unit comprises no —COOH groups or a salt form thereof.

In some embodiments, a 5′-end group is or comprises a phosphate group, or a derivative or a bioisostere thereof. In some embodiments, a 5′-nucleoside unit comprises a 5′-group which is a phosphate group, or a derivative or a bioisostere thereof. As appreciated by a person having ordinary skill in the art, a number of such groups are known in the art and can be utilized in accordance with the present disclosure.

In some embodiments, a 5′-end group is —CH₂—O—P(O)(OH)—(OH) or a salt form thereof.

In some embodiments, a provided 5′-nucleoside unit has the structure of

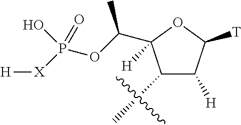

or a salt form thereof. In some embodiments, a provided 5′-nucleoside unit has the structure of

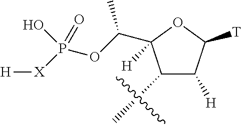

or a salt form thereof. In some embodiments, X is O. In some embodiments, X is S. In some embodiments, R^{E} is —(R)—CH(CH₃)—O—P(O)(OH)—S—H or a salt form thereof. In some embodiments, R^{E} is —(R)—CH(CH₃)—O—P(O)(OH)—O—H or a salt form thereof. In some embodiments, R^{E} is —(S)—CH(CH₃)—O—P(O)(OH)—S—H or a salt form thereof. In some embodiments, R^{E} is —(S)—CH(CH₃)—O—P(O)(OH)—O—H or a salt form thereof. In some embodiments, a provided 5′-nucleoside unit has the structure of

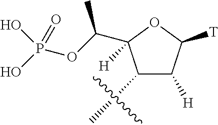

or a salt form thereof. In some embodiments, a provided 5′-nucleoside unit has the structure of

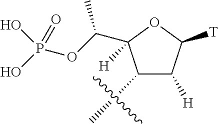

or a salt form thereof.

As readily appreciated by a person having ordinary skill in the art, provided compounds, e.g., oligonucleotides, or partial structures thereof, e.g., 5′-end structures, internucleotidic linkages, etc. of oligonucleotides, may partially, sometimes predominantly, exist as one or more salt forms thereof at certain pH, e.g., physiological pH, for example, due to one or more acidic and/or basic moieties therein. In some embodiments, a provided 5′-nucleoside unit may partially, sometimes predominately, exist as one or more its salt forms. For example, depending on pH,

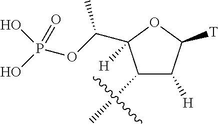

may exist as

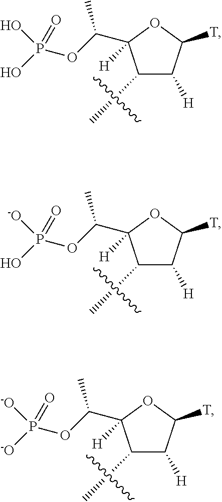

or any combinations thereof. Unless explicitly specified otherwise, all salt forms are included when provided compounds or structures are recited.

In some embodiments, R^{E} is -L-P(O)(XR)₂ or a salt form thereof. In some embodiments, R^{E} is -L-P(O)(XR)₂ or a salt form thereof, wherein each X is independently —O—, —S—, or a covalent bond. In some embodiments, R^{E} is -L-P(O)(OR)₂ or a salt form thereof. In some embodiments, R^{E} is -L-P(O)(OR)(SR) or a salt form thereof. In some embodiments, R^{E} is -L-P(O)(OR)(R) or a salt form thereof. In some embodiments, L is a covalent bond, or a bivalent, optionally substituted, linear or branched C₁₋₆ aliphatic, wherein one or more methylene units are optionally and independently replaced with —O—, —S— or —N(R′)—. In some embodiments, R^{E} is -L-R^{5s}. In some embodiments, R^{E} is —X-L-R. In some embodiments, R^{E} is

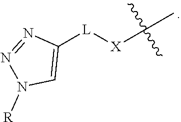

In some embodiments, X in R^{E} is —C(R)₂—. In some embodiments, X is —O—. In some embodiments, X is —S—. In some embodiments, X is —N(R)—. In some embodiments, L comprises an optionally substituted, bivalent or multivalent

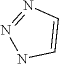

group. In some embodiments, L comprises an optionally substituted

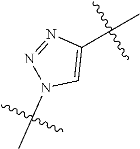 [SMILES: *c1cn(*)nn1]

group. In some embodiments, L comprises a

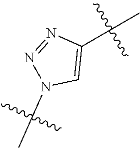 [SMILES: *c1cn(*)nn1]

group. In some embodiments, R is independently —H, or an optionally substituted group selected from C₁₋₁₀ alkyl, C₁₋₁₀ allyl, and C₆₋₁₄ aryl. In some embodiments, R is —H. In some embodiments, R^{E} is optionally substituted

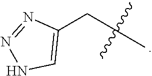 [SMILES: *Cc1c[nH]nn1]

In some embodiments, R^{E} is

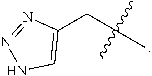 [SMILES: *Cc1c[nH]nn1]

Many phosphate derivatives and/or bioisosteres, and 5′-nucleoside units are described in literature and can be utilized in accordance with the present disclosure, for example, some such structures are described in, e.g., US 2016-0194349; US 2016-0186175; US 20130323836, etc. In some embodiments, a 5′ end group R^{E}, or a 5′-nucleoside unit, is described in, for example, Allerson et al. 2005 J. Med. Chem. 48: 901-04; Lima et al. 2012 Cell 150: 883-894; Prakash et al. 2015 Nucl. Acids Res. 43: 2993-3011; and/or Prakash et al. 2016 Bioorg. Med. Chem. Lett. 26: 26: 2817-2820, for example, T-VP, T-PO, etc.

Bridged Morpholinos and cyclohexenyl nucleotides and nucleosides are described in, for example, US patent application publication 2016-0186175, which can be utilized in accordance with the present disclosure.

Example embodiments of variables are extensively described in the present disclosure. For structures with two or more variables, unless otherwise specified, each variable can independently be any embodiment described herein.

In some embodiments, an APOC3 oligonucleotide capable of directing a decrease in the expression and/or level of a target gene or its gene product can comprise any 5′-end described herein or known in the art.

In some embodiments, an APOC3 oligonucleotide, an APOC3 oligonucleotide that directs RNA interference, an APOC3 oligonucleotide that directs RNase H-mediated knockdown can comprise any 5′-end described herein or known in the art.

In some embodiments, an APOC3 oligonucleotide, an APOC3 oligonucleotide that directs RNase H-mediated knockdown, or an APOC3 oligonucleotide that directs both RNA interference and RNase H-mediated knockdown can comprise any 5′-end described herein or known in the art.

In some embodiments, the 5′-end of a provided single-stranded RNAi agent comprises a phosphorus-comprising moiety (e.g., a 5′-end comprises a phosphorus). Non-limiting examples of ssRNAi formats wherein the 5′-end comprises a phosphorus-comprising moiety include Formats 1-15, 20-21, 23-31, 80-82, 92-95, 97-102, and 104-107 of FIG. 1.

In some embodiments, the 5′-end of a provided single-stranded RNAi agent does not comprise a phosphorus-comprising moiety (e.g., a 5′-end comprises a phosphorus). Non-limiting examples of ssRNAi formats wherein the 5′-end does not comprise a phosphorus-comprising moiety include Formats 16-19, 22, 32-79, 83-91, 96, and 103 of FIG. 1.

In some embodiments, the 5′-end of a provided single-stranded RNAi agent comprises a moiety comprising a phosphate, such as a phosphodiester, phosphorothioate, phosphorodithioate, H-phosphonate, or other moiety similar or identical to a phosphate-comprising internucleotidic linkage. In some embodiments, the 5′-end of a provided single-stranded RNAi agent comprises a moiety comprising a phosphate, but which is not a phosphodiester; such a moiety in some embodiments is referred to as a phosphate mimic, modified phosphate or phosphate analog.

In some embodiments, the 5′-end of a provided single-stranded RNAi agent does not comprise a moiety comprising a phosphate.

In some embodiments of a single-stranded RNAi agent, a 5′ end structure has a structure selected from: 5′-(R)-Me OH T, 5′-(R)-Me PO T, 5′-(R)-Me PS T, 5′-(R)-Me PH T, 5′-(S)-Me OH T, 5′-(S)-Me PO T, 5′-(S)-Me PS T, and 5′-(S)—PH T.

In some embodiments of a provided single-stranded RNAi agent, which comprise a phosphorus-comprising moiety at the 5′-end structure which is represented by a structure selected from the Formula IV-a (Mod022 also known as C3 PO and n-propyl), IV-b (Mod022*), IV-c (POMod023*), IV-d (PSMod023*), and IV-e (PHMod023*):

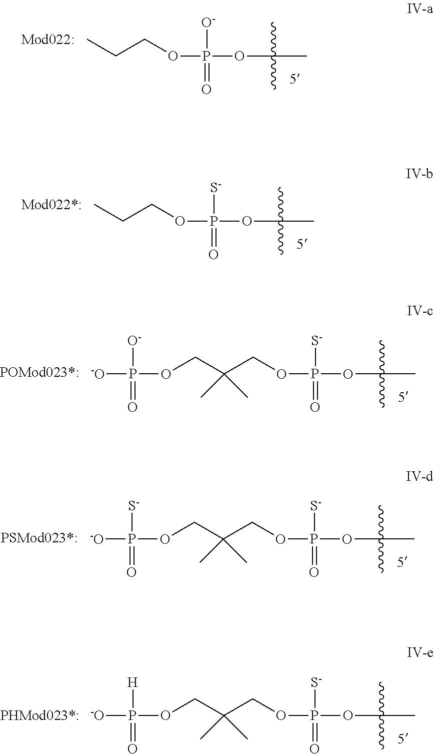

In some embodiments of a provided single-stranded RNAi agent, which comprise a phosphorus-comprising moiety at the 5′-end structure is represented by a structure selected of the structure of Formula IV-f (also known as n-propyl, C3 PO or Mod022):

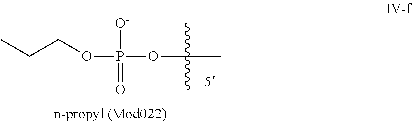 [SMILES: *OP(=O)([O-])OCCC]

wherein 5′ indicates the attachment point to the 5′ carbon of a sugar.

In some embodiments of a provided single-stranded RNAi agent, which comprise a phosphorus-comprising moiety at the 5′-end structure is represented by a structure selected of the following structure (also known as C3 PS or Mod022*):

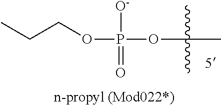 [SMILES: *OP(=O)([O-])OCCC]

wherein 5′ indicates the attachment point to the 5′ carbon of a sugar (e.g., of N1).

In some embodiments of a provided single-stranded RNAi agent, which comprise a phosphorus-comprising moiety at the 5′-end structure is represented by a structure selected of the structure of Formula IV-g (also known as DimethylC3 or C3dimethyl PS or Mod023*):

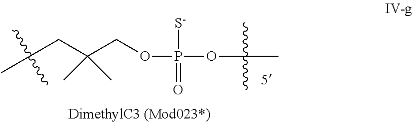 [SMILES: *CC(C)(C)COP(=O)([S-])O*]

wherein 5′ indicates the attachment point to the 5′ carbon of a sugar (e.g., of N1).

In some embodiments, a single-stranded RNAi agent comprises a 5′-end structure which is selected from any of PO (phosphorodiester), Formula IV-h; PH (H-Phosphonate), Formula IV-i; and PS (Phosphorothioate), Formula IV-j:

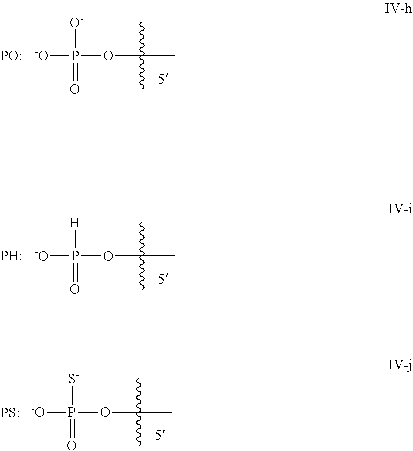

In some embodiments of a provided single-stranded RNAi agent, which comprise a phosphorus-comprising moiety at the 5′-end structure is represented by a structure selected from any of the following:

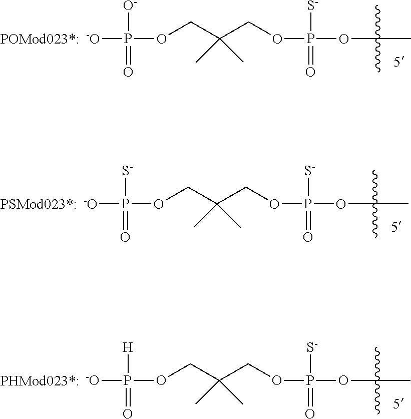

wherein 5′ indicates the attachment point to the 5′ carbon of a sugar (e.g., of N1).

In some embodiments, P in any of Formula IV-a to IV-j is stereorandom or stereodefined as in the Sp or Rp configuration.

In some embodiments, a 5′-end structure is selected from any of: a phosphate, a phosphate analogue, 5′-monophosphate ((HO)2(O)P—O-5′), 5′-diphosphate ((HO)2(O)P—O—P(HO)(O)—O-5′-triphosphate ((HO)2(O)P—O—(HO)(O)P—O—P(HO)(O)—O-5′), 5′-guanosine cap (7-methylated or non-methylated) (7m-G-O-5′-(HO)(O)P—O—(HO)(O)P—O—P(HO)(O)—O-5′), 5′-adenosine cap (Appp), and any modified or unmodified nucleotide cap structure (N—O-5′-(HO)(O)P—O—(HO)(O)P—O—P(HO)(O)—O-5′), 5′-monothiophosphate (phosphorothioate; (HO)2(S)P—O-5′), 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)2(O)P—NH-5′, (HO)(NH2)(O)P—O-5′), 5′-alkylphosphonates (R=alkyl=methyl, ethyl, isopropyl, propyl, etc., e.g. RP(OH)(O)—O-5′-, (OH)2(O)P-5′-CH₂—), 5′-alkyletherphosphonates (R=alkylether=methoxymethyl (MeOCH2-), ethoxymethyl, etc., e.g. RP(OH)(O)—O-5′-).

In some embodiments, a 5′-end comprising a phosphorus-comprising moiety can have particular advantages, in that the single-stranded RNAi agents comprising them may be more active in RNA interference.

In some embodiments, a 5′ end structure has a structure of a 5′-nucleotide or a modified 5′-nucleotide, a 5′-nucleotide analog, a 5′-nucleoside or a modified 5′-nucleoside or a 5′-nucleoside analog.

In some embodiments, a 5′ end structure, has a structure of any of: a 5′-guanosine cap, a 5′-adenosine cap, a 5′-monothiophosphate, a 5′-monodithiophosphate, a 5′-phosphorothiolate, a 5′-phosphoramidate, a 5′-alkylphosphonate, and a 5′-alkyletherphosphonate; a 5′-monophosphate, a 5′-diphosphate, and a 5′-triphosphate; 5′-triphosphate; a monophosphate, a diphosphate, or a triphosphate in which at least one oxygen atom of the monophosphate, diphosphate, or triphosphate is replaced with a sulfur atom; 5′-alpha-thiotriphosphate and 5′-gamma-thiotriphosphate; alkylphosphonate; alkylphosphonate has the formula: RP(OH)(O)—O-5′ or (OH)₂(O)P-5′—CH₂—, wherein R is a C₁-C₃ alkyl; alkyletherphosphonate; or alkyletherphosphonate of the formula: RP(OH)(O)—O-5′, wherein R is an alkylether.

Various 5′-nucleosides are described in, for example, U.S. patent application Ser. No. 14/959,714, published as US 2016-0194349 A1; U.S. patent application Ser. No. 14/983,907, published as US 2016-0186175 A1; or U.S. patent application Ser. No. 13/696,796, published as US 20130323836.

In some embodiments, a 5′-end structure is a vinylphosphonate.

In certain embodiments, oligomeric compounds are provided wherein said 5′-terminal compound has Formula VIII-c wherein G is F, OCH₃ or O(CH₂)₂—OCH₃.

In some embodiments, a 5′ end structure has a structure selected from: 5′-(R)-Me OH T, 5′-(R)-Me PO T, 5′-(R)-Me PS T, 5′-(R)-Me PH T, 5′-(S)-Me OH T, 5′-(S)-Me PO T, 5′-(S)-Me PS T, and 5′-(S)—PH T.

In some embodiments, a 5′ end structure has the structure of 5′-(R)-Me OH T.

In some embodiments, a 5′ end structure has the structure of 5′-(R)-Me PO T.

In some embodiments, a 5′ end structure has the structure of 5′-(R)-Me PS T.

In some embodiments, a 5′ end structure has the structure of 5′-(R)-Me PH T.

In some embodiments, a 5′ end structure has the structure of 5′-(S)-Me OH T.

In some embodiments, a 5′ end structure has the structure of 5′-(S)-Me PO T.

In some embodiments, a 5′ end structure has the structure of 5′-(S)-Me PS T.

In some embodiments of a single-stranded RNAi agent, a 5′ end structure has the structure of 5′-(R)-Me PO T.

In some embodiments of a single-stranded RNAi agent, a 5′ end structure has the structure of 5′-(R)-Me PS T.

In some embodiments of a single-stranded RNAi agent, a 5′ end structure has the structure of 5′-(R)-Me PH T.

In some embodiments of a single-stranded RNAi agent, a 5′ end structure has the structure of 5′-(S)-Me OH T.

In some embodiments of a single-stranded RNAi agent, a 5′ end structure has the structure of 5′-(S)-Me PO T.

In some embodiments of a single-stranded RNAi agent, a 5′ end structure has the structure of 5′-(S)-Me PS T.

In some embodiments of a single-stranded RNAi agent, a 5′ end structure has the structure of 5′-(S)-Me PH T.

In some embodiments, a 5′ end structure has the structure of 5′-(S)-Me PH T. In addition, some references such as EP 1520022 B1, paragraph 6, have reported that a 5′ phosphate is required at the target-complementary strand (e.g., the antisense strand) of a siRNA duplex for RISC activity. U.S. Pat. No. 8,729,036, column 2, also noted that 5′ phosphates are reported to be essential for RNA interference. U.S. Pat. No. 8,729,036, column 3, also reported that a 5′ phosphate was required for single-stranded antisense siRNAs to trigger RNAi in HeLa S100 extract. However, the present disclosure has demonstrated that various single-stranded RNAi agents which do not comprise a 5′ phosphate are capable of directing RNA interference.

In some embodiments, a 5′-end comprises a phosphate-comprising moiety such as T-VP or T-PO, or any other suitable RNAi agent 5′-end compound as described in, for example, Allerson et al. 2005 J. Med. Chem. 48: 901-04; Lima et al. 2012 Cell 150: 883-894; Prakash et al. 2015 Nucl. Acids Res. 43: 2993-3011; and/or Prakash et al. 2016 Bioorg. Med. Chem. Lett. 26: 26: 2817-2820.

In some embodiments, a 5′-end which does not comprise a phosphorus-comprising moiety can have particular advantages, in that the single-stranded RNAi agent can be easier to synthesize, and it may not be necessary to protect the phosphorus-comprising moiety from degradation. In some embodiments, a 5′-end of a provided single-stranded RNAi agent which does not comprise a phosphorus-comprising moiety comprises a moiety which can act as a substrate for a mammalian kinase which, inside a target cell, is able to attach a phosphorus-comprising moiety at the 5′-end of the single-stranded RNAi agent.

In some embodiments, a 5′-end does not comprise a phosphorus-comprising moiety.

In some embodiments of a single-stranded RNAi agent, the single-stranded RNAi agent comprises a 5′ terminal OH, and the first nucleoside is 2′-deoxy.

In some embodiments of a single-stranded RNAi agent, the single-stranded RNAi agent comprises a 5′ terminal OH, and the first nucleoside is 2′-deoxy T.

In some embodiments of a single-stranded RNAi agent, the single-stranded RNAi agent comprises a 5′ terminal OH, and the first nucleoside is 2′-deoxy U.

In some embodiments of a single-stranded RNAi agent, the single-stranded RNAi agent comprises a 5′ terminal OH, and the first nucleoside is 2′-deoxy A.

In some embodiments of a single-stranded RNAi agent, the single-stranded RNAi agent comprises a 5′ terminal OH, and the first nucleoside is 2′-deoxy G.

In some embodiments of a single-stranded RNAi agent, the single-stranded RNAi agent comprises a 5′ terminal OH, and the first nucleoside is 2′-deoxy C.

In some embodiments of a single-stranded RNAi agent, the single-stranded RNAi agent comprises a 5′ terminal OH, and the first nucleoside is 2′-deoxy.

In some embodiments of a single-stranded RNAi agent, the single-stranded RNAi agent comprises a 5′ terminal OH, and the first nucleoside is 2′-deoxy T. In some embodiments of a single-stranded RNAi agent, the single-stranded RNAi agent comprises a 5′ terminal OH, and the first nucleoside is 2′-deoxy U.

In some embodiments of a single-stranded RNAi agent, the single-stranded RNAi agent comprises a 5′ terminal OH, and the first nucleoside is 2′-deoxy A. In some embodiments of a single-stranded RNAi agent, the single-stranded RNAi agent comprises a 5′ terminal OH, and the first nucleoside is 2′-deoxy G.

In some embodiments of a single-stranded RNAi agent, the single-stranded RNAi agent comprises a 5′ terminal OH, and the first nucleoside is 2′-deoxy C.

In some embodiments of a single-stranded RNAi agent, the single-stranded RNAi agent comprises a 5′ terminal OH, and the first nucleoside is 2′-OMe.

In some embodiments of a single-stranded RNAi agent, the single-stranded RNAi agent comprises a 5′ terminal OH, and the first nucleoside is 2′-OMe T.

In some embodiments of a single-stranded RNAi agent, the single-stranded RNAi agent comprises a 5′ terminal OH, and the first nucleoside is 2′-OMe U.

In some embodiments of a single-stranded RNAi agent, the single-stranded RNAi agent comprises a 5′ terminal OH, and the first nucleoside is 2′-OMe A.

In some embodiments of a single-stranded RNAi agent, the single-stranded RNAi agent comprises a 5′ terminal OH, and the first nucleoside is 2′-OMe G.

In some embodiments of a single-stranded RNAi agent, the single-stranded RNAi agent comprises a 5′ terminal OH, and the first nucleoside is 2′-OMe C.

In some embodiments of a single-stranded RNAi agent, the single-stranded RNAi agent comprises a 5′ terminal OH, and the first nucleoside is 2′-OMe.

In some embodiments of a single-stranded RNAi agent, the single-stranded RNAi agent comprises a 5′ terminal OH, and the first nucleoside is 2′-OMe T.

In some embodiments of a single-stranded RNAi agent, the single-stranded RNAi agent comprises a 5′ terminal OH, and the first nucleoside is 2′-OMe U. In some embodiments of a single-stranded RNAi agent, the single-stranded RNAi agent comprises a 5′ terminal OH, and the first nucleoside is 2′-OMe A.

In some embodiments of a single-stranded RNAi agent, the single-stranded RNAi agent comprises a 5′ terminal OH, and the first nucleoside is 2′-OMe G.

In some embodiments of a single-stranded RNAi agent, the single-stranded RNAi agent comprises a 5′ terminal OH, and the first nucleoside is 2′-OMe C.

In some embodiments of a single-stranded RNAi agent, the single-stranded RNAi agent comprises a 5′ terminal OH, and the first nucleoside is 2′-F.

In some embodiments of a single-stranded RNAi agent, the single-stranded RNAi agent comprises a 5′ terminal OH, and the first nucleoside is 2′-F T.

In some embodiments of a single-stranded RNAi agent, the single-stranded RNAi agent comprises a 5′ terminal OH, and the first nucleoside is 2′-F U.

In some embodiments of a single-stranded RNAi agent, the single-stranded RNAi agent comprises a 5′ terminal OH, and the first nucleoside is 2′-F A.

In some embodiments of a single-stranded RNAi agent, the single-stranded RNAi agent comprises a 5′ terminal OH, and the first nucleoside is 2′-F G.

In some embodiments of a single-stranded RNAi agent, the single-stranded RNAi agent comprises a 5′ terminal OH, and the first nucleoside is 2′-F C.

In some embodiments of a single-stranded RNAi agent, the single-stranded RNAi agent comprises a 5′ terminal OH, and the first nucleoside is 2′-F.

In some embodiments of a single-stranded RNAi agent, the single-stranded RNAi agent comprises a 5′ terminal OH, and the first nucleoside is 2′-F T.

In some embodiments of a single-stranded RNAi agent, the single-stranded RNAi agent comprises a 5′ terminal OH, and the first nucleoside is 2′-F U.

In some embodiments of a single-stranded RNAi agent, the single-stranded RNAi agent comprises a 5′ terminal OH, and the first nucleoside is 2′-F A. In some embodiments of a single-stranded RNAi agent, the single-stranded RNAi agent comprises a 5′ terminal OH, and the first nucleoside is 2′-F G. In some embodiments of a single-stranded RNAi agent, the single-stranded RNAi agent comprises a 5′ terminal OH, and the first nucleoside is 2′-F C.

In some embodiments of a single-stranded RNAi agent, the single-stranded RNAi agent comprises a 5′ terminal phosphate, and the first nucleoside is 2′-deoxy.

In some embodiments of a single-stranded RNAi agent, the single-stranded RNAi agent comprises a 5′ terminal phosphate, and the first nucleoside is 2′-deoxy T.

In some embodiments of a single-stranded RNAi agent, the single-stranded RNAi agent comprises a 5′ terminal phosphate, and the first nucleoside is 2′-deoxy U.

In some embodiments of a single-stranded RNAi agent, the single-stranded RNAi agent comprises a 5′ terminal phosphate, and the first nucleoside is 2′-deoxy A.

In some embodiments of a single-stranded RNAi agent, the single-stranded RNAi agent comprises a 5′ terminal phosphate, and the first nucleoside is 2′-deoxy G.

In some embodiments of a single-stranded RNAi agent, the single-stranded RNAi agent comprises a 5′ terminal phosphate, and the first nucleoside is 2′-deoxy C.

In some embodiments of a single-stranded RNAi agent, the single-stranded RNAi agent comprises a 5′ terminal phosphate, and the first nucleoside is 2′-deoxy.

In some embodiments of a single-stranded RNAi agent, the single-stranded RNAi agent comprises a 5′ terminal phosphate, and the first nucleoside is 2′-deoxy T.

In some embodiments of a single-stranded RNAi agent, the single-stranded RNAi agent comprises a 5′ terminal phosphate, and the first nucleoside is 2′-deoxy U.

In some embodiments of a single-stranded RNAi agent, the single-stranded RNAi agent comprises a 5′ terminal phosphate, and the first nucleoside is 2′-deoxy A. In some embodiments of a single-stranded RNAi agent, the single-stranded RNAi agent comprises a 5′ terminal phosphate, and the first nucleoside is 2′-deoxy G.

In some embodiments of a single-stranded RNAi agent, the single-stranded RNAi agent comprises a 5′ terminal phosphate, and the first nucleoside is 2′-deoxy C.

In some embodiments of a single-stranded RNAi agent, the single-stranded RNAi agent comprises a 5′ terminal phosphate, and the first nucleoside is 2′-F.

In some embodiments of a single-stranded RNAi agent, the single-stranded RNAi agent comprises a 5′ terminal phosphate, and the first nucleoside is 2′-F T.

In some embodiments of a single-stranded RNAi agent, the single-stranded RNAi agent comprises a 5′ terminal phosphate, and the first nucleoside is 2′-F U.

In some embodiments of a single-stranded RNAi agent, the single-stranded RNAi agent comprises a 5′ terminal phosphate, and the first nucleoside is 2′-F A.

In some embodiments of a single-stranded RNAi agent, the single-stranded RNAi agent comprises a 5′ terminal phosphate, and the first nucleoside is 2′-F G.

In some embodiments of a single-stranded RNAi agent, the single-stranded RNAi agent comprises a 5′ terminal phosphate, and the first nucleoside is 2′-F C.

In some embodiments of a single-stranded RNAi agent, the single-stranded RNAi agent comprises a 5′ terminal phosphate, and the first nucleoside is 2′-F.

In some embodiments of a single-stranded RNAi agent, the single-stranded RNAi agent comprises a 5′ terminal phosphate, and the first nucleoside is 2′-F T.

In some embodiments of a single-stranded RNAi agent, the single-stranded RNAi agent comprises a 5′ terminal phosphate, and the first nucleoside is 2′-F U. In some embodiments of a single-stranded RNAi agent, the single-stranded RNAi agent comprises a 5′ terminal phosphate, and the first nucleoside is 2′-F A. In some embodiments of a single-stranded RNAi agent, the single-stranded RNAi agent comprises a 5′ terminal phosphate, and the first nucleoside is 2′-F G.

In some embodiments of a single-stranded RNAi agent, the single-stranded RNAi agent comprises a 5′ terminal phosphate, and the first nucleoside is 2′-F C.

In some embodiments of a single-stranded RNAi agent, the single-stranded RNAi agent comprises a 5′ terminal phosphate, and the first nucleoside is 2′-OMe.

In some embodiments of a single-stranded RNAi agent, the single-stranded RNAi agent comprises a 5′ terminal phosphate, and the first nucleoside is 2′-OMe T.

In some embodiments of a single-stranded RNAi agent, the single-stranded RNAi agent comprises a 5′ terminal phosphate, and the first nucleoside is 2′-OMe U.

In some embodiments of a single-stranded RNAi agent, the single-stranded RNAi agent comprises a 5′ terminal phosphate, and the first nucleoside is 2′-OMe A.

In some embodiments of a single-stranded RNAi agent, the single-stranded RNAi agent comprises a 5′ terminal phosphate, and the first nucleoside is 2′-OMe G.

In some embodiments of a single-stranded RNAi agent, the single-stranded RNAi agent comprises a 5′ terminal phosphate, and the first nucleoside is 2′-OMe C.

In some embodiments of a single-stranded RNAi agent, the single-stranded RNAi agent comprises a 5′ terminal phosphate, and the first nucleoside is 2′-OMe. In some embodiments of a single-stranded RNAi agent, the single-stranded RNAi agent comprises a 5′ terminal phosphate, and the first nucleoside is 2′-OMe T.

In some embodiments of a single-stranded RNAi agent, the single-stranded RNAi agent comprises a 5′ terminal phosphate, and the first nucleoside is 2′-OMe U. In some embodiments of a single-stranded RNAi agent, the single-stranded RNAi agent comprises a 5′ terminal phosphate, and the first nucleoside is 2′-OMe A. In some embodiments of a single-stranded RNAi agent, the single-stranded RNAi agent comprises a 5′ terminal phosphate, and the first nucleoside is 2′-OMe G.

In some embodiments of a single-stranded RNAi agent, the single-stranded RNAi agent comprises a 5′ terminal phosphate, and the first nucleoside is 2′-OMe C.

In some embodiments, an APOC3 oligonucleotide comprises an additional component which binds to ASPGR. In some embodiments, the additional component is on the 5′ end of the oligonucleotide.

In some embodiments, an APOC3 oligonucleotide comprises an additional component which is or comprises a compound of Formula (K)

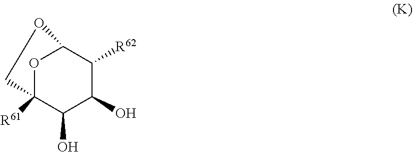

wherein R is —CN, —CH₂—CN, —C≡CH, —CH₂—N₃, —CH₂—NH₂, —CH₂—N(R)—S(O)₂—R, —CH₂—CO₂H, —CO₂H, —CH₂—OH, —CH₂—SH, —CH═CH—R, —CH₂—R, —CH₂—S—R, —CH₂—N(R)—R, —CH₂—N(R)—C(O)—R, —CH₂—N(R)—C(O)—O—R, —CH₂—N(R)—C(O)—N(R)—R, —CH₂—O—R, —CH₂—O—C(O)—R, —C(O)—N(R)—R, —CH₂—O—C(O)—O—R, —CH₂—S(O)—R, —CH₂—S(O)₂—R, —CH₂—S(O)₂—N(R)—R, —C(O)—NH₂, —C(O)—O—R, —C(O)—N(R)—R, or aryl or heteroaryl, wherein the aryl or heteroaryl is optionally substituted with R,
or R is —Z—X—Y wherein X is a linker or a drug delivery system, Y is absent or is a ligand selected from the group consisting of a small molecule, an amino acid sequence, a nucleic acid sequence, an antibody, an oligomer, a polymer, genetically derived material, a liposome, a nanoparticle, dye, fluorescent probe, or a combination thereof, and Z is absent or is —C≡C—, —CH═CH—, —CH₂—, —CH₂—O—, —C(O)—N(R)—, —CH₂—S(O)—, —CH₂—S(O)₂—, —CH₂—S(O)₂—N(R)—, —C(O)—O—, —CH₂—N(R)—, —CH₂—N(R)—C(O)—, —CH₂—N(R)—S(O)₂—, —CH₂—N(R)—C(O)—O—, —CH₂—N(R)—C(O)—N(R)—, —CH₂—O—C(O)—, —CH₂—O—C(O)—N(R)—, —CH₂—O—C(O)—O—, or aryl or heteroaryl, wherein the aryl or heteroaryl is optionally substituted with R;
R⁶² is —OH, —N₃, —N(R)₂, —N(R)—C(O)—R′—N(R)—C(O)—N(R)_{2′}—N(R)—C(O)—OR, tetrazole, or triazole, wherein the tetrazole and triazole are optionally substituted with R,
and wherein when R is —CH₂—OH, R is —N₃, —N(R)₂, —N(R)—C(O)—R′—N(R)—C(O)—N(R)₂—N(R)—C(O)—OR, tetrazole, or triazole, wherein the tetrazole and triazole are optionally substituted with R;
each R is independently —H, halo-substituted (C₁-C₅)alkyl, or (C₃-C₆)cycloalkyl, wherein a —CH₂— group of the alkyl or cycloalkyl may be replaced with a heteroatom group selected from —O—, —S—, and —N(R)— and —CH₃ of the alkyl may be replaced with a heteroatom group selected from —N(R)2, —OR, and —S(R) wherein the heteroatom groups are separated by at least 2 carbon atoms;
each R is independently —H, —(C₁-C₂₀)alkyl, or (C₃-C₆)cycloalkyl wherein one to six —CH₂— groups of the alkyl or cycloalkyl separated by at least two carbon atoms may be replaced with —O—, —S—, or —N(R)—, and —CH₃ of the alkyl may be replaced with a heteroatom group selected from —N(R)₂, —OR, and —S(R) wherein the heteroatom groups are separated by at least 2 carbon atoms; and wherein the alkyl and cycloalkyl may be substituted with one to six halo atoms; and
each R is independently —H, (C₃-C₂₀)cycloalkyl or (C₁-C₂₀)alkyl wherein one to six —CH₂— groups of the alkyl or cycloalkyl separated by at least two carbon atoms may be replaced with —O—, —S—, or —N(R)—, and —CH₃ of the alkyl may be replaced with a heteroatom group selected from —N(R)₂, —OR, and —S(R) wherein the heteroatom groups are separated by at least 2 carbon atoms; and wherein the alkyl and cycloalkyl may be substituted with one to six halo atoms.

In some embodiments, an APOC3 oligonucleotide comprises an additional component which is or comprises a compound of Formula (M)

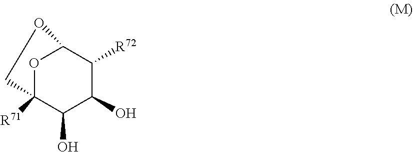

wherein R is —CN, —CH₂—CN, —C≡CH, —CH₂—N₃, —CH₂—NH₂, —CH₂—N(R)—S(O)₂—R, —CH₂—CO₂H, —CO₂H, —CH₂—OH, —CH₂— SH, —CH═CH—R, —CH₂—R, —CH₂—S—R, —CH₂—N(R)—R, —CH₂—N(R)—C(O)—R, —CH₂—N(R)—C(O)—O—R, —CH₂—N(R)—C(O)—N(R)—R, —CH₂—O—R, —CH₂—O—C(O)—R, —CH₂—O—C(O)—N(R)—R, —CH₂—O—C(O)—O—R, —CH₂—S(O)—R, —CH₂—S(O)₂—R, —CH₂—S(O)₂—N(R)—R, —C(O)—NH₂, —C(O)—O—R, —C(O)—N(R)—R, or aryl or heteroaryl, wherein the aryl or heteroaryl is optionally substituted with R,
or R is —Z—X—Y, —Z—Y, —X—Y, —X, —Y, or —Z—X wherein X is a linker or a drug delivery system, Y is R or is a ligand selected from the group consisting of a small molecule, an amino acid sequence, a nucleic acid sequence, an antibody, an oligomer, a polymer, genetically derived material, a liposome, a nanoparticle, dye, fluorescent probe, or a combination thereof, and Z is —C≡C—, —CH═CH—, —CH₂—, —CH₂—O—, —C(O)—N(R)—, —CH₂—S—, —CH₂—S(O)—, —CH₂—S(O)₂—, —CH₂—S(O)₂—N(R)—, —C(O)—O—, —CH₂—N(R)—, —CH₂—N(R)—C(O)—, —CH₂—N(R)—S(O)2-, —CH₂—N(R)—C(O)—O—, —CH₂—N(R)—C(O)—N(R)—, —CH₂—O—C(O)—, —CH₂—O—C(O)—N(R)—, —CH₂—O—C(O)—O—, or aryl or heteroaryl, wherein the aryl or heteroaryl is optionally substituted with R;
R is —OH, —N₃, —N(R)₂, —N(R)—C(O)—R′—N(R)—C(O)—N(R)_{2′}—N(R)—C(O)—OR, —N(R)—S(O)₂—R′ tetrazole, or triazole, wherein the tetrazole and triazole are optionally substituted with R
and wherein when R is —CH₂—OH, R is —N₃, —N(R)₂, —N(R)—C(O)—R′—N(R)—C(O)—N(R)_{2′}—N(R)—C(O)—OR, N(R)—S(O)₂—R, tetrazole, or triazole, wherein the tetrazole and triazole are optionally substituted with R;
each R is independently —H, —(C₁-C₅)alkyl, halo-substituted (C₁-C₅)alkyl, or (C₃-C₆)cycloalkyl, wherein one or more —CH₂— groups of the alkyl or cycloalkyl may each be replaced with a heteroatom group independently selected from —O—, —S—, and —N(R)— and —CH₃ of the alkyl may be replaced with a heteroatom group selected from —N(R)₂, —OR, and —S(R) wherein the heteroatom groups are separated by at least 2 carbon atoms;
each R is independently —H, —(C₁-C₂₀)alkyl, or (C₃-C₆)cycloalkyl wherein one to six —CH₂— groups of the alkyl or cycloalkyl separated by at least two carbon atoms may each be replaced with a heteroatom independently selected from —O—, —S—, or —N(R)—, and —CH₃ of the alkyl may be replaced with a heteroatom group selected from —N(R)₂, —OR, and —S(R) wherein the heteroatom groups are separated by at least 2 carbon atoms; and wherein the alkyl and cycloalkyl may be substituted with halo atoms;
each R is independently —H, (C₃-C₂₀)cycloalkyl or (C₁-C₆₀)alkyl wherein one to six —CH₂— groups of the cycloalkyl or one to 20—CH₂— groups of the alkyl may each be replaced with heteroatoms independently selected from —O—, —S—, and —N(R)— wherein the heteroatoms are separated by at least two carbon atoms, and —CH₃ of the alkyl may be replaced with a heteroatom group selected from —N(R)2, —OR, and —S(R) wherein the heteroatom groups are separated by at least 2 carbon atoms; and wherein the alkyl and cycloalkyl may be substituted with halo atoms; and
each R is independently H, —C═CH₂, —CH₃, —N₃, —N(R)₂, —OH, —S(O)—(R), —S(O)₂—(R), —C(O)—OH, —S—S-aryl, —S—S-heteroaryl, heterocycyl, aryl or heteroaryl, wherein each aryl or heteroaryl is optionally substituted with R.

In some embodiments, R⁶¹ or R⁷¹ is —X—Y, and/or R⁶² or R⁷² is —NH—C(O)—CH₃.

In some embodiments, an APOC3 oligonucleotide comprises an additional component selected from the group consisting of:
benzyl (4-((2-((1-(1-((1S,2R,3R,4R,5S)-4-acetamido-2,3-dihydroxy-6,8-dioxabicyclo[3.2.1]octan-1-yl)-2,5,8,11-tetraoxatridecan-13-yl)-1H-1,2,3-triazol-4-yl)methoxy)ethyl)amino)-4-oxobutyl)carbamate,
benzyl (4-((1,3-bis((1-(1-((1S,2R,3R,4R,5S)-4-acetamido-2,3-dihydroxy-6,8-dioxabicyclo[3.2.1]octan-1-yl)-2,5,8,11-tetraoxatridecan-13-yl)-1H-1,2,3-triazol-4-yl)methoxy)propan-2-yl)amino)-4-oxobutyl)carbamate,
benzyl (4-((1,3-bis((1-(1-((1S,2R,3R,4R,5S)-4-acetamido-2,3-dihydroxy-6,8-dioxabicyclo[3.2.1]octan-1-yl)-2,5,8,11-tetraoxatridecan-13-yl)-1H-1,2,3-triazol-4-yl)methoxy)-2-(((1-(1-((1S,2R,3R,4R,5S)-4-acetamido-2,3-dihydroxy-6,8-dioxabicyclo[3.2.1]octan-1-yl)-2,5,8,11-tetraoxatridecan-13-yl)-1H-1,2,3-triazol-4-yl)methoxy)methyl)propan-2-yl)amino)-4-oxobutyl)carbamate,
N-(2-((1-(1-((1S,2R,3R,4R,5S)-4-acetamido-2,3-dihydroxy-6,8-dioxabicyclo[3.2.1]octan-1-yl)-2,5,8,11-tetraoxatridecan-13-yl)-1H-1,2,3-triazol-4-yl)methoxy)ethyl)-4-aminobutanamide,
4-amino-N-{1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]propan-2-yl}butanamide,
4-amino-N-(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5 S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)butanamide,
4-amino-N-[1,31-bis(1-{[(1S,2R,3R,4R,5S)-4-(acetylamino)-2,3-dihydroxy-6,8-dioxabicyclo[3.2.1]oct-1-yl]methyl}-1H-1,2,3-triazol-4-yl)-2,6,10,14,18,22,26,30-octaoxahentriacontan-16-yl]butanamide,
4-amino-N-{1,31-bis(1-{[(1S,2R,3R,4R,5 S)-4-(acetylamino)-2,3-dihydroxy-6,8-dioxabicyclo[3.2.1]oct-1-yl]methyl}-1H-1,2,3-triazol-4-yl)-16-[15-(1-{[(1S,2R,3R,4R,5S)-4-(acetylamino)-2,3-dihydroxy-6,8-dioxabicyclo[3.2.1]oct-1-yl]methyl}-1H-1,2,3-triazol-4-yl)-2,6,10,14-tetraoxapentadec-1-yl]-2,6,10,14,18,22,26,30-octaoxahentriacontan-16-yl}butanamide,
N-{(1S,2R,3R,4R,5S)-1-[(hexyloxy)methyl]-2,3-dihydroxy-6,8-dioxabicyclo[3.2.1]oct-4-yl}acetamide,
N-[(1S,2R,3R,4R,5S)-2,3-dihydroxy-1-(2,5,8,11,14-pentaoxapentadec-1-yl)-6,8-dioxabicyclo[3.2.1]oct-4-yl]acetamide,
N-((1S,2R,3R,4R,5S)-2,3-dihydroxy-1-(hydroxymethyl)-6,8-dioxabicyclo[3.2.1]octan-4-yl)-2,2,2-trifluoroacetamide, compound,
N-[(1S,2R,3R,4R,5S)-2,3-dihydroxy-1-(hydroxymethyl)-6,8-dioxabicyclo[3.2.1]oct-4-yl]-2,2,2-trifluoroacetamide,
N-[(1S,2R,3R,4R,5S)-2,3-dihydroxy-1-(hydroxymethyl)-6,8-dioxabicyclo[3.2.1]oct-4-yl]propanamide,
N-[(1S,2R,3R,4R,5S)-2,3-dihydroxy-1-(hydroxymethyl)-6,8-dioxabicyclo[3.2.1]oct-4-yl]methanesulfonamide,
N-[(1S,2R,3R,4R,5S)-2,3-dihydroxy-1-(hydroxymethyl)-6,8-dioxabicyclo[3.2.1]oct-4-yl]-2,2-difluoroacetamide,
N-[(1S,2R,3R,4R,5S)-2,3-dihydroxy-1-(hydroxymethyl)-6,8-dioxabicyclo[3.2.1]oct-4-yl]-3,3,3-trifluoropropanamide,
N-[(1S,2R,3R,4R,5S)-2,3-dihydroxy-1-(hydroxymethyl)-6,8-dioxabicyclo[3.2.1]oct-4-yl]-N-methylacetamide,
N-[(1S,2R,3R,4R,5S)-2,3-dihydroxy-1-(hydroxymethyl)-6,8-dioxabicyclo[3.2.1]oct-4-yl]-N-methylmethanesulfonamide,
tert-butyl [(1S,2R,3R,4R,5S)-2,3-dihydroxy-1-(hydroxymethyl)-6,8-dioxabicyclo[3.2.1]oct-4-yl]methylcarbamate,
(1S,2R,3R,4R,5S)-1-(hydroxymethyl)-4-(methylamino)-6,8-dioxabicyclo[3.2.1]octane-2,3-diol hydrochloride,
N-[(1S,2R,3R,4R,5S)-2,3-dihydroxy-1-(15-phenyl-2,5,8,11,14-pentaoxapentadec-1-yl)-6,8-dioxabicyclo[3.2.1]oct-4-yl]acetamide,
N-[(1S,2R,3R,4R,5S)-1-(13-azido-2,5,8,11-tetraoxatridec-1-yl)-2,3-dihydroxy-6,8-dioxabicyclo[3.2.1]oct-4-yl]acetamide,
N-[(1S,2R,3R,4R,5S)-2,3-dihydroxy-1-(2,5,8,11-tetraoxatetradec-13-en-1-yl)-6,8-dioxabicyclo[3.2.1]oct-4-yl]acetamide,
N-[(1S,2R,3R,4R,5S)-2,3-dihydroxy-1-(2,5,8,11-tetraoxatetradec-13-yn-1-yl)-6,8-dioxabicyclo[3.2.1]oct-4-yl]acetamide,
N-[(1S,2R,3R,4R,5S)-1-(13-amino-2,5,8,11-tetraoxatridec-1-yl)-2,3-dihydroxy-6,8-dioxabicyclo[3.2.1]oct-4-yl]acetamide,
N-[(1S,2R,3R,4R,5S)-2,3-dihydroxy-1-(13-hydroxy-2,5,8,11-tetraoxatridec-1-yl)-6,8-dioxabicyclo[3.2.1]oct-4-yl]acetamide,
1-[(1S,2R,3R,4R,5S)-4-(acetylamino)-2,3-dihydroxy-6,8-dioxabicyclo[3.2.1]oct-1-yl]-2,5,8,11-tetraoxatridecan-13-oic acid,
S-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}ethanethioate,
N-{(1S,2R,3R,4R,5S)-2,3-dihydroxy-1-[13-(pyridin-2-yldisulfanyl)-2,5,8,11-tetraoxatridec-1-yl]-6,8-dioxabicyclo[3.2.1]oct-4-yl}acetamide,
N-(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)-6-(pyridin-2-yldisulfanyl)hexanamide,
N-[(1S,2R,3R,4R,5S)-1-(13-{4-[(3-[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl-2-aminopropoxy)methyl]-1H-1,2,3-triazol-1-yl}-2,5,8,11-tetraoxatridec-1-yl)-2,3-dihydroxy-6,8-dioxabicyclo[3.2.1]oct-4-yl]acetamide-hydrochloric acid salt,
6-azido-N-(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)hexanamide,
N-(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)hept-6-enamide,
N-(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)hept-6-ynamide,
7-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-7-oxoheptanoic acid (Sodium salt),
benzyl {6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}carbamate,
6-amino-N-(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)hexanamide acetate salt,
N-{6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}-6-(2,5-dioxo-2,5-dihydro-1H-pyrrol-1-yl)hexanamide,
N-{6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl-6-[(bromoacetyl)amino]hexanamide,
4-{[(2R)-5-(carbamoylamino)-2-{[(2R)-2-cyclopentyl-2-{[6-(2,5-dioxo-2,5-dihydro-1H-pyrrol-1-yl)hexanoyl]amino}acetyl]amino}pentanoyl]amino}benzyl {6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}carbamate,
N-(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)-3,19-dioxo-1-(pyridin-2-yldisulfanyl)-7,10,13,16-tetraoxa-4,20-diazahexacosan-26-amide,
N-(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)-3,31-dioxo-1-(pyridin-2-yldisulfanyl)-7,10,13,16,19,22,25,28-octaoxa-4,32-diazaoctatriacontan-38-amide,
N-{6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}-6-(pyridin-2-yldisulfanyl)hexanamide,
2-(pyridin-2-yldisulfanyl)ethyl {6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}carbamate,
N-{6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}-6-(4,4,5,5-tetramethyl-1,3,2-dioxaborolan-2-yl)hexanamide,
N-{6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}-N-(1,3-dihydroxypropan-2-yl)heptanediamide,
6-azido-N-{6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}hexanamide,
6-(benzyloxy)-N-{6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}hexanamide,
(1S,2R,3R,4R,5S)-4-(acetylamino)-1-{13-[4-({3-[(1-{1-[(1S,2R,3R,4R,5S)-4-(acetylamino)-2,3-bis(acetyloxy)-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-bis(acetyloxy)-6,8-dioxabicyclo[3.2.1]oct-1-yl]-2,5,8,11-tetraoxatridecan-13-yl}-1H-1,2,3-triazol-4-yl)methoxy]methyl}-2-({6 [(6hydroxyhexanoyl)amino]hexanoyl}amino)propoxy}methyl)-1H-1,2,3-triazol-1-yl]-2,5,8,11-tetraoxatridec-1-yl}-3-(acetyloxy)-6,8-dioxabicyclo[3.2.1]oct-2-yl acetate,
benzyl [6-({6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}amino)-6-oxohexyl]carbamate,
6-amino-N-{6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}hexanamide acetate,
4-(benzyloxy)-N-(1,3-bis[(1-{1-[(1S,2R,3R,4R,5S)-4-amino-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-amino-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)butanamide,
N-(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)-4-hydroxybutanamide, and
N-(2-{[6-({6-[(1,3-bis[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]-2-{[(1-{1-[(1S,2R,3R,4R,5S)-4-(acetylamino)-2,3-dihydroxy-6,8-dioxabicyclo[3.2.1]oct-1-yl]-2,5,8,11-tetraoxatridecan-13-yl}-1H-1,2,3-triazol-4-yl)methoxy]methyl}propan-2-yl)amino]-6-oxohexyl}amino)-6-oxohexyl]oxy}-1,3-dioxan-5-yl)-6-(pyridin-2-yldisulfanyl)hexanamide

In some embodiments, an APOC3 oligonucleotide comprises an additional component of Formula (N)

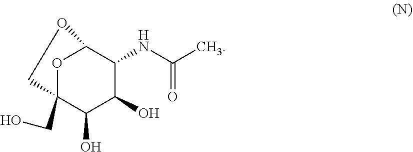 [SMILES: CC(=O)N[C@H]1[C@H]2OC[C@](CO)(O2)[C@H](O)[C@@H]1O]

In some embodiments, an APOC3 oligonucleotide comprises an additional component selected from:

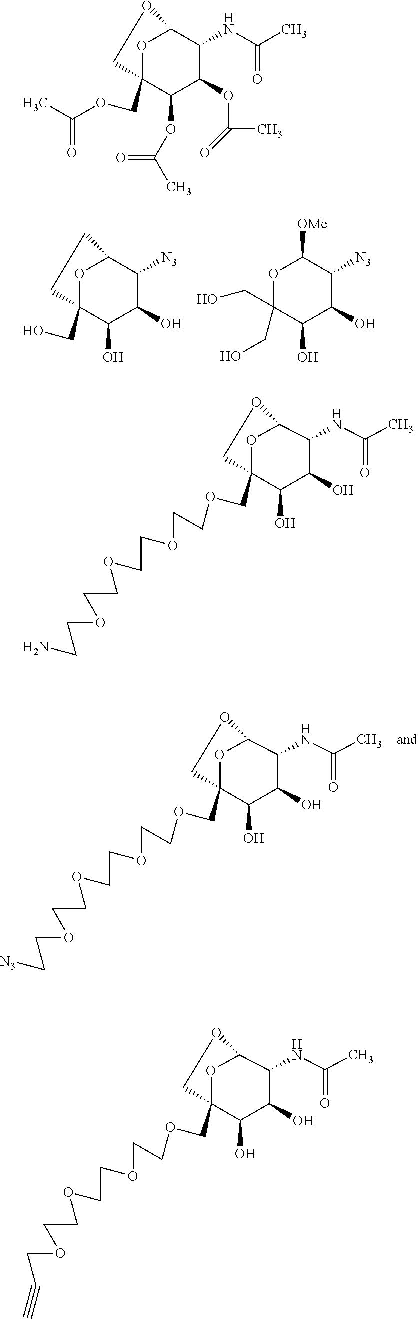 [SMILES: CC(=O)N[C@H]1[C@H]2OC[C@](COC(C)=O)(O2)[C@H](OC(C)=O)[C@@H]1OC(C)=O ; [N-]=[N+]=N[C@@H]1[C@@H](O)[C@@H](O)[C@]2(CO)CC[C@H]1O2 ; CO[C@@H]1OC(CO)(CO)[C@H](O)[C@H](O)[C@H]1N=[N+]=[N-] ; CC(=O)N[C@H]1[C@H]2OC[C@](COCCOCCOCCOCCN)(O2)[C@H](O)[C@@H]1O ; CC(=O)N[C@H]1[C@H]2OC[C@](COCCOCCOCCOCCN=[N+]=[N-])(O2)[C@H](O)[C@@H]1O ; C#CCOCCOCCOCCOC[C@@]12CO[C@@H](O1)[C@H](NC(C)=O)[C@@H](O)[C@H]2O]

In some embodiments, an APOC3 oligonucleotide comprises an additional component selected from any of the following formulae:

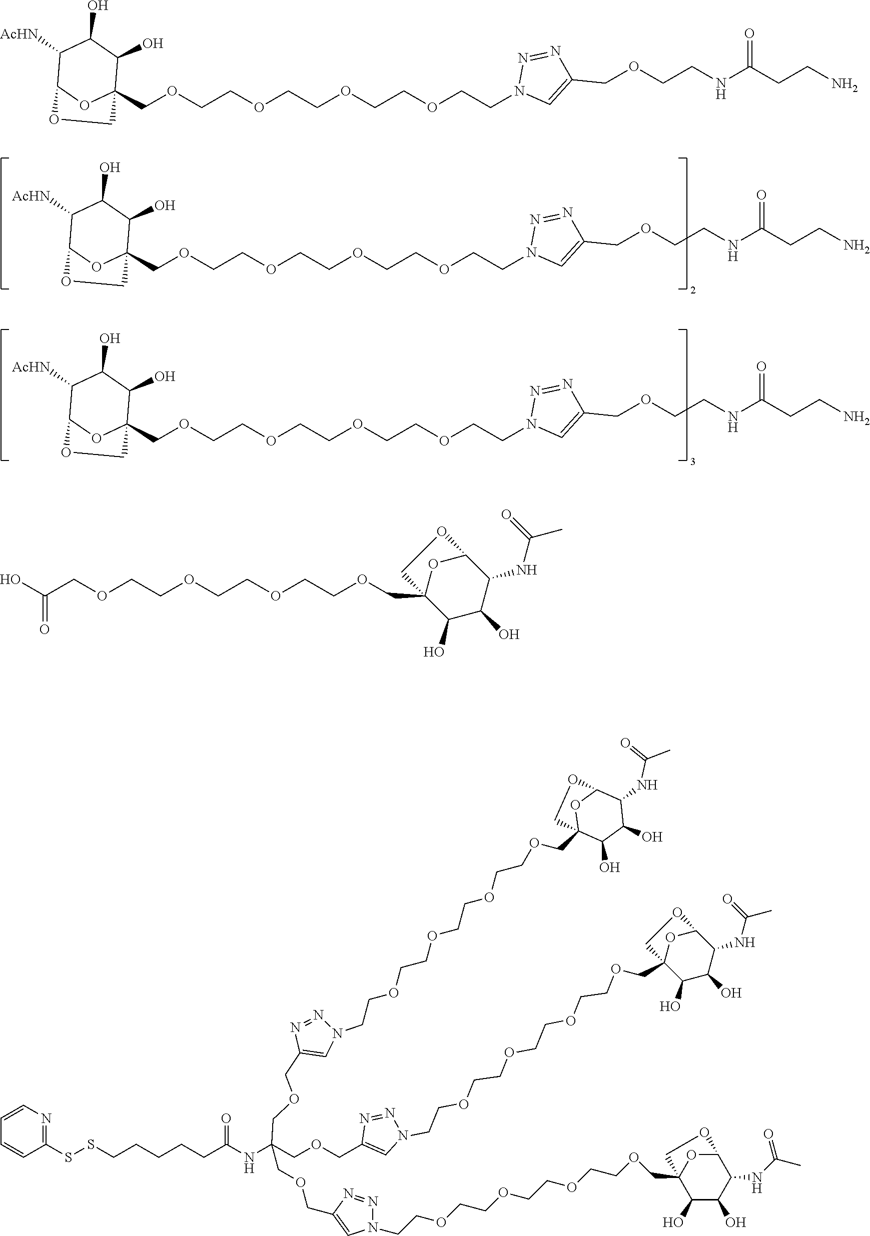

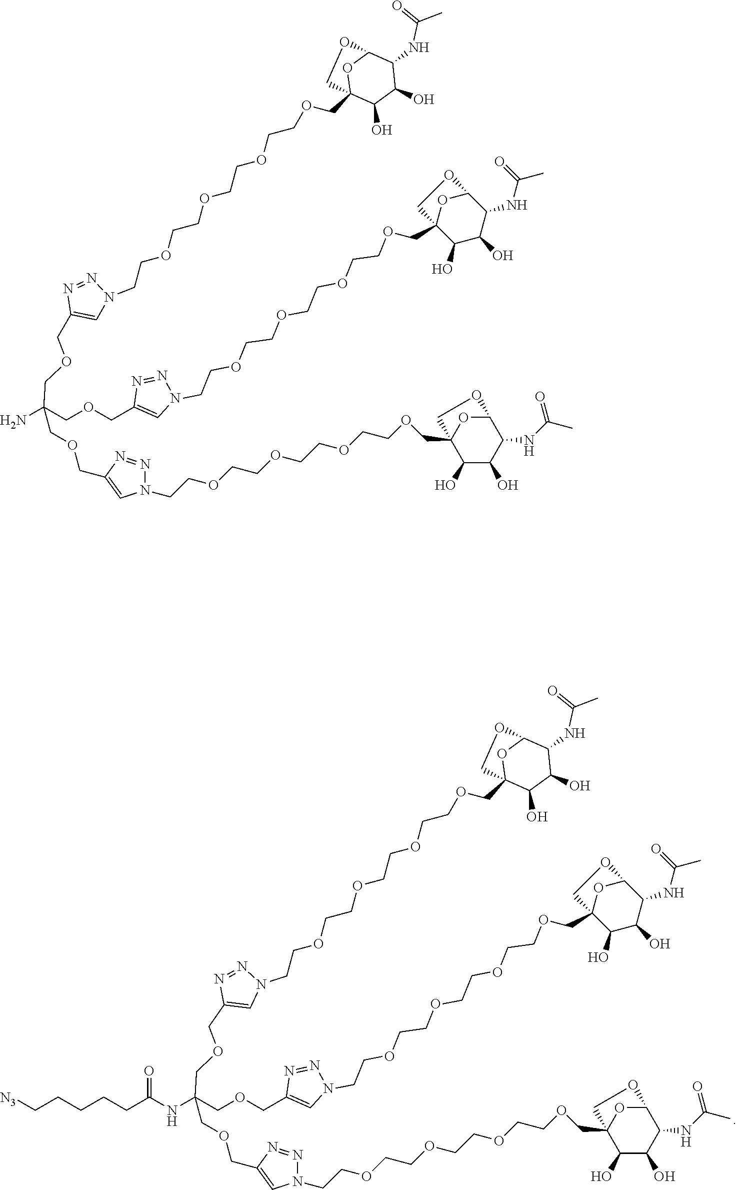 [SMILES: CC(=O)N[C@H]1[C@H]2OC[C@](COCCOCCOCCOCCn3cc(COCC(N)(COCc4cn(CCOCCOCCOCCOC[C@@]56CO[C@@H](O5)[C@H](NC(C)=O)[C@@H](O)[C@H]6O)nn4)COCc4cn(CCOCCOCCOCCOC[C@@]56CO[C@@H](O5)[C@H](NC(C)=O)[C@@H](O)[C@H]6O)nn4)nn3)(O2)[C@H](O)[C@@H]1O ; CC(=O)N[C@H]1[C@H]2OC[C@](COCCOCCOCCOCCn3cc(COCC(COCc4cn(CCOCCOCCOCCOC[C@@]56CO[C@@H](O5)[C@H](NC(C)=O)[C@@H](O)[C@H]6O)nn4)(COCc4cn(CCOCCOCCOCCOC[C@@]56CO[C@@H](O5)[C@H](NC(C)=O)[C@@H](O)[C@H]6O)nn4)NC(=O)CCCCCN=[N+]=[N-])nn3)(O2)[C@H](O)[C@@H]1O]

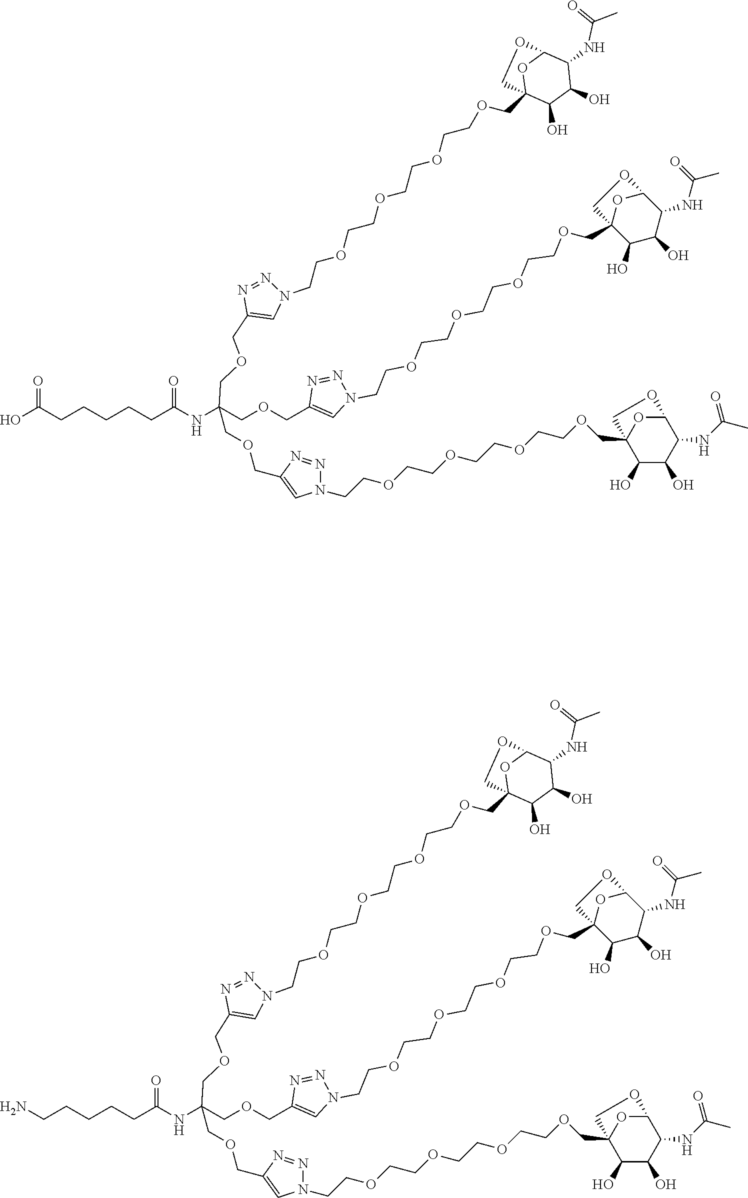 [SMILES: CC(=O)N[C@H]1[C@H]2OC[C@](COCCOCCOCCOCCn3cc(COCC(COCc4cn(CCOCCOCCOCCOC[C@@]56CO[C@@H](O5)[C@H](NC(C)=O)[C@@H](O)[C@H]6O)nn4)(COCc4cn(CCOCCOCCOCCOC[C@@]56CO[C@@H](O5)[C@H](NC(C)=O)[C@@H](O)[C@H]6O)nn4)NC(=O)CCCCCC(=O)O)nn3)(O2)[C@H](O)[C@@H]1O ; CC(=O)N[C@H]1[C@H]2OC[C@](COCCOCCOCCOCCn3cc(COCC(COCc4cn(CCOCCOCCOCCOC[C@@]56CO[C@@H](O5)[C@H](NC(C)=O)[C@@H](O)[C@H]6O)nn4)(COCc4cn(CCOCCOCCOCCOC[C@@]56CO[C@@H](O5)[C@H](NC(C)=O)[C@@H](O)[C@H]6O)nn4)NC(=O)CCCCCN)nn3)(O2)[C@H](O)[C@@H]1O]

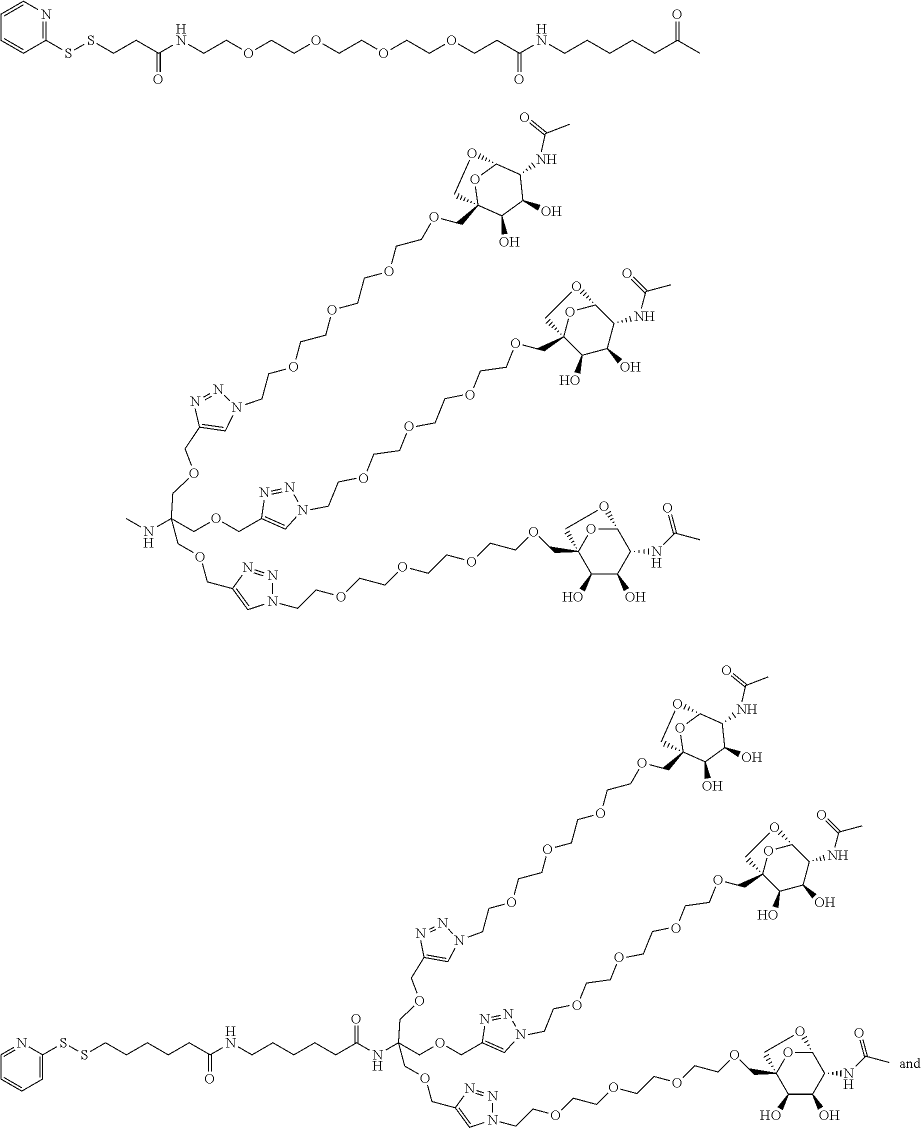

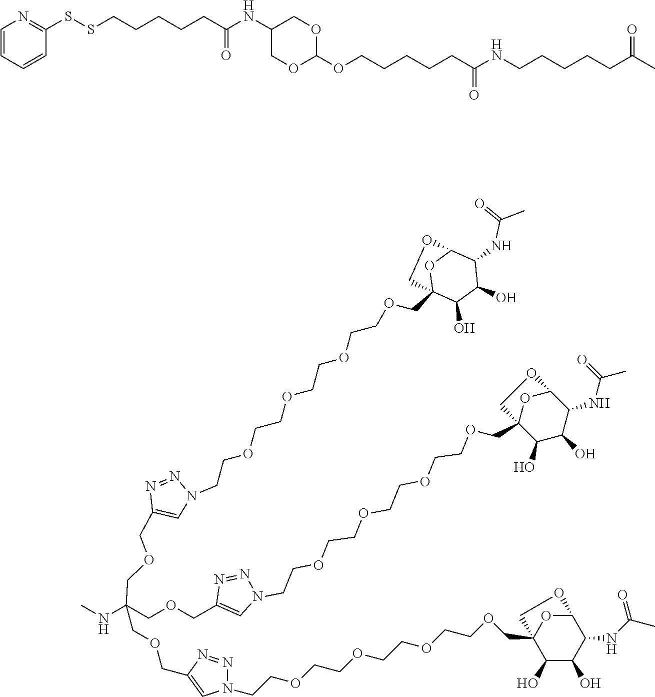

In some embodiments, the present disclosure pertains to: a compound having the Formula O1:

Y¹-L¹-(Z¹⁰)_{za} O1

or a pharmaceutically acceptable salt of said compound wherein Y¹ is an oligonucleotide targeting APOC3;

za is 1, 2, or 3; and

L¹ is a compound of Formula L11, L12, L13, L43, L44, L45, L46, L47, L48, L49, L50, L51, L52, L53 or L54 wherein the connection sites with Y¹ and Z¹⁰ are indicated:

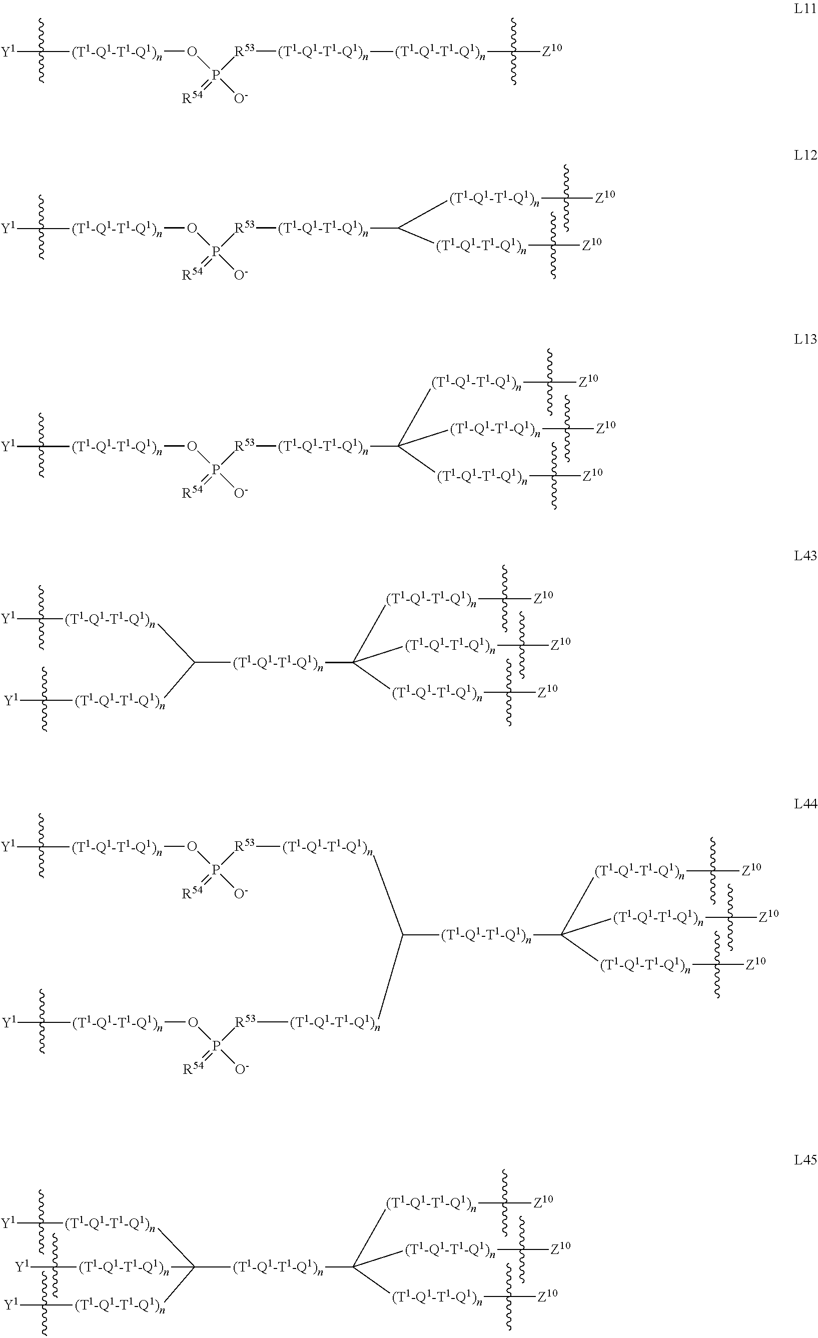

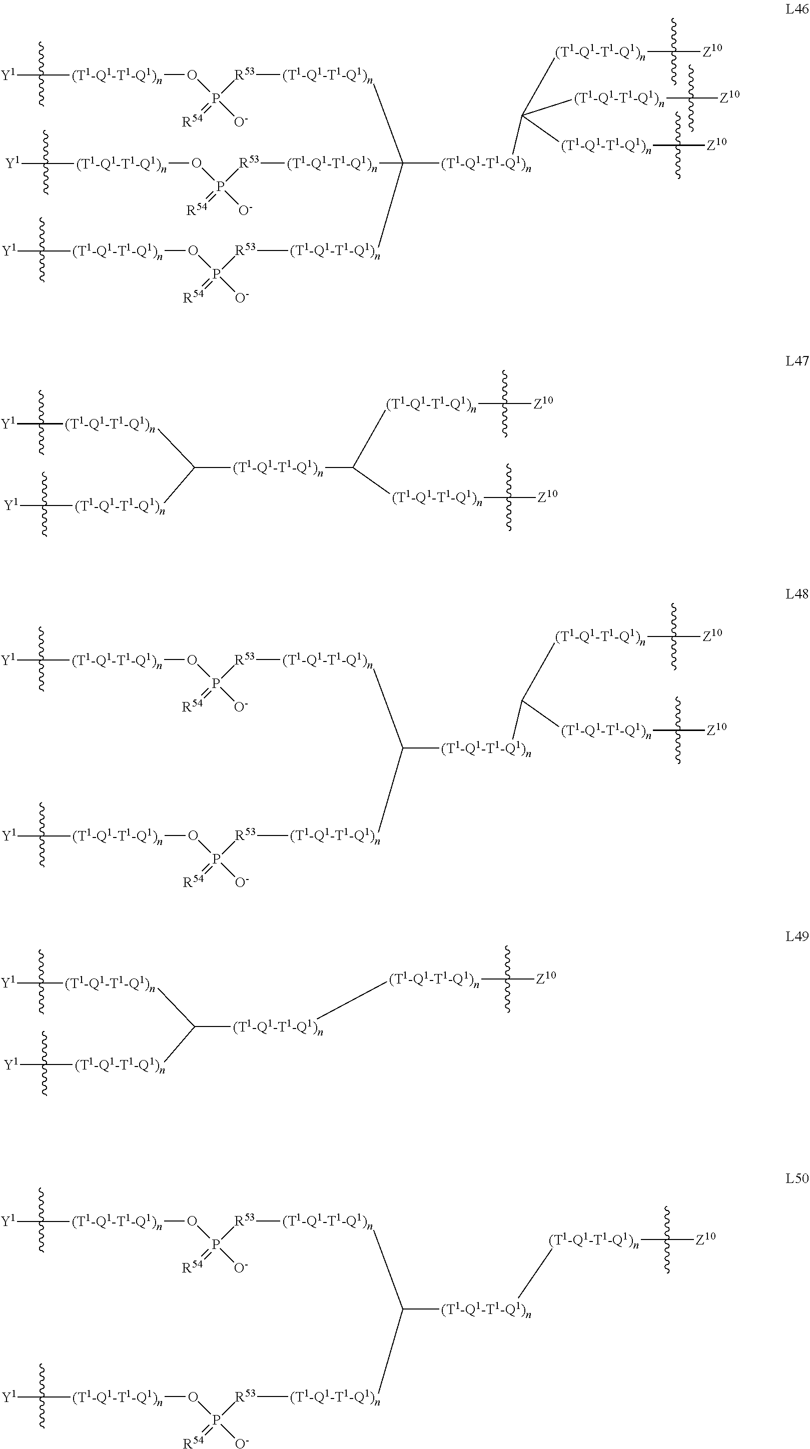

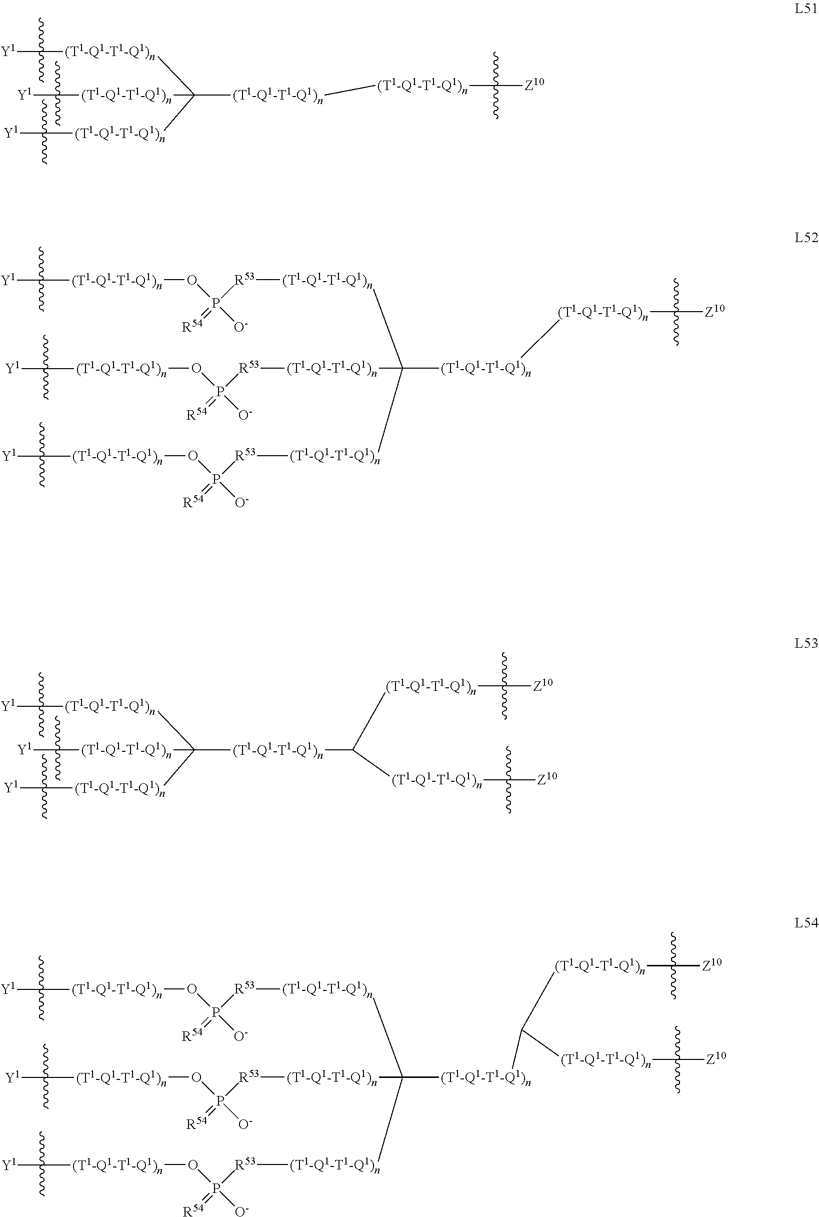

wherein each T¹ is independently absent or is alkylene, alkenylene, or alkynylene, wherein one or more —CH₂— groups of the alkylene, alkenylene, or alkynylene may each independently be replaced with a heteroatom group independently selected from —O—, —S—, and —N(R⁴⁹)— wherein the heteroatom groups are separated by at least 2 carbon atoms;

each Q¹ is independently absent or is —C(O)—, —C(O)—NR⁴⁹—, —NR⁴⁹—C(O)—, —O—C(O)—NR⁴⁹—, —NR⁴⁹—C(O)—O—, —CH₂—, —NR⁴⁹C(O)NR⁴⁹—, a bivalent heteroaryl group, or a heteroatom group selected from —O—, —S—, —S—S—, —S(O)—, —S(O)₂—, and —NR⁴⁹—, wherein at least two carbon atoms separate the heteroatom groups —O—, —S—, —S—S—, —S(O)—, —S(O)₂— and —NR⁴⁹— from any other heteroatom group, or a structure of the formula:

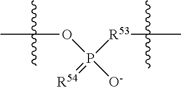

wherein R⁵³ is —O or —NH—, and R⁵⁴ is —O or —S;

each R⁴⁹ is independently —H, —(C₁-C₂₀)alkyl, or —(C₃-C₆)cycloalkyl wherein one to six —CH₂— groups of the alkyl or cycloalkyl separated by at least two carbon atoms may be replaced with —O—, —S—, or —N(R^{49a})—, and —CH₃ of the alkyl may be replaced with a heteroatom group selected from —N(R^{49a})₂, —OR^{49a}, and —S(R^{49a}) wherein the heteroatom groups are separated by at least 2 carbon atoms; and wherein the alkyl and cycloalkyl may be substituted with halo atoms and wherein each R^{49a} is independently —H, —(C₁-C₆)alkyl, or —(C₃-C₆)cycloalkyl;

R⁵³ is —O or —NH;

R⁵⁴ is —O or —S;

each n is independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; wherein if n is greater than 0, each T¹ and each Q¹ of each (T¹-Q¹-T¹-Q¹) is independently selected; and

each Z¹⁰ is independently a compound of Formula Z12, Z13, Z14, Z15, Z16, Z17, Z18, Z19, Z20, or Z21, or a geometrical or position isomer thereof, wherein the connection site with L¹ is indicated:

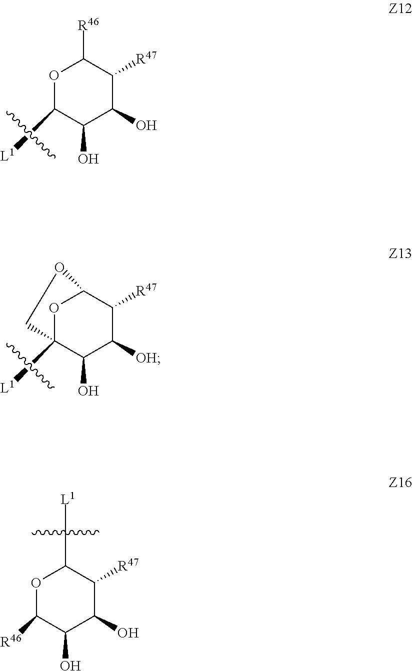

wherein each R⁴⁶ is independently —CN, —CH₂—CN, —C≡CH, —CH₂—N₃, —CH₂—NH₂, —CH₂—N(R⁵²)—S(O)₂—R⁵¹, —CH₂—CO₂H, —CO₂H, —CH₂—OH, —CH₂—SH, —CH═CH—R⁵¹, —CH₂—R⁵¹, —CH₂—S—R⁵¹, —CH₂—N(R⁵²)—R⁵¹, —CH₂—N(R⁵²)—C(O)—R⁵¹, —CH₂—N(R⁵²)—C(O)—O—R⁵¹, —CH₂—N(R⁵²)—C(O)—N(R⁵²)—R⁵¹, —CH₂—O-R⁵¹, —CH₂—O—C(O)—R ⁵¹, —CH₂—O—C(O)—N(R⁵²)—R⁵¹, —CH₂—O—C(O)—O—R⁵¹, —CH₂—S(O)—R⁵¹, —CH₂—S(O)₂—R⁵¹, —CH₂—S(O)₂—N(R⁵²)—R⁵¹, —C(O)—NH₂, —C(O)—O—R⁵¹, —C(O)—N(R⁵²)—R⁵¹, or aryl or heteroaryl, wherein the aryl or heteroaryl is optionally substituted with R⁵¹

each R⁴⁷ is independently —OH, —N3, —N(R⁴⁸)₂, —N(R⁴⁸)—C(O)—R⁴⁸, —N(R⁴⁸)—C(O)—N(R⁴⁸)₂, —N(R⁴⁸)—C(O)—OR⁴⁸, —N(R⁴⁸)—S(O)₂—R⁴⁸, tetrazole, or triazole, wherein the tetrazole and triazole are optionally substituted with R⁴⁸;

each R⁴⁸ is independently —H, —(C₁-C₅)alkyl, halo-substituted (C₁-C₅)alkyl, halo substituted —(C₃-C₆)cycloalkyl, —(C₁-C₅)alkenyl, —(C₁-C₅)alkynyl, halo substituted —(C₁-C₅)alkenyl, halo substituted —(C₁-C₅)alkynyl, or —(C₃-C₆)cycloalkyl, wherein a —CH₂— group of the alkyl or cycloalkyl may each be independently replaced with a heteroatom group selected from —O—, —S—, and —N(R⁵²)— and —CH₃ of the alkyl may each be independently replaced with a heteroatom group selected from —N(R⁵²)₂, —OR⁵², and —S(R⁵²) wherein the heteroatom groups are separated by at least 2 carbon atoms;

each R⁵¹ is independently —H, —(C₃-C₂₀)cycloalkyl, —(C₁-C₆₀)alkenyl, —(C₁-C₆₀)alkynyl, or —(C₁-C₆₀)alkyl wherein one to six —CH₂— groups of the cycloalkyl or one to 20—CH₂— groups of the alkyl may each be independently replaced with heteroatoms independently selected from —O—, —S—, and —N(R⁴⁹)— wherein the heteroatoms are separated by at least two carbon atoms, and —CH₃ of the alkyl may each be independently replaced with a heteroatom group selected from —N(R⁴⁹)₂, —OR⁴⁹, and —S(R⁴⁹) wherein the heteroatom groups are separated by at least 2 carbon atoms, and wherein the alkyl, alkenyl, alkynyl, and cycloalkyl may be substituted with halo atoms; and

each R⁵² is independently —H, —(C₁-C₂₀)alkyl, —(C₁-C₂₀)alkenyl, —(C₁-C₂₀)alkynyl, or —(C₃-C₆)cycloalkyl wherein one to six —CH₂— groups of the alkyl or cycloalkyl separated by at least two carbon atoms may each be independently replaced with a heteroatom independently selected from —O—, —S—, or —N(R⁴⁹)—, and —CH₃ of the alkyl may each be independently replaced with a heteroatom group selected from —N(R⁴⁹)₂, —OR⁴⁹, and —S(R⁴⁹) wherein the heteroatom groups are separated by at least 2 carbon atoms; and wherein the alkyl, alkenyl, alkynyl, and cycloalkyl may be substituted with halo atoms.

In some embodiments, Y¹ comprises at least 15 bases.

In some embodiments, the base sequence of Y¹ comprises or is the base sequence of any APOC3 oligonucleotide listed in Table 1A, or the base sequence of Y¹ comprises 15 contiguous bases of the sequence of any APOC3 oligonucleotide listed in Table 1A.

In some embodiments, Y¹ comprises at least 1 phosphodiester internucleotidic linkage.

In some embodiments, Y¹ comprises at least 1 chirally controlled modified internucleotidic linkage.

In some embodiments, Y¹ comprises at least 1 chirally controlled modified internucleotidic linkage which is a chirally controlled phosphorothioate.

In some embodiments, Y¹ comprises at least 1 chirally controlled modified internucleotidic linkage which is a chirally controlled phosphorothioate in the Sp configuration.

In some embodiments, Y¹ comprises at least 1 chirally controlled modified internucleotidic linkage which is a chirally controlled phosphorothioate in the Rp configuration.

In some embodiments, Y¹, wherein the chirally controlled modified internucleotidic linkage or chirally controlled phosphorothioate comprises a phosphorus chiral center which has a diastereopurity of at least 70% within the composition.

In some embodiments, Y¹, wherein the chirally controlled modified internucleotidic linkage or chirally controlled phosphorothioate comprises a phosphorus chiral center which has a diastereopurity of at least 70%, 75%, 80%, 85%, 90%, 91%, 92%, 93%, 94%, 95%, 96%, 97%, 98%, 99% or 99.5%.

In some embodiments, Y¹ comprises at least 1 sugar modification.

In some embodiments, Y¹ comprises at least 1 base modification.

In some embodiments, Y¹ further comprises a pattern of backbone linkages.

In some embodiments, Y¹ further comprises a pattern of backbone chiral centers.

In some embodiments, Y¹ further comprises a pattern of chemical modifications.

In some embodiments, Y¹ further comprises a pattern of backbone linkages, a pattern of backbone chiral centers, and a pattern of chemical modifications.

In some embodiments, the pattern of backbone linkages, the pattern of backbone chiral centers, and the pattern of chemical modifications of the oligonucleotide are the pattern of backbone linkages, the pattern of backbone chiral centers, and/or the pattern of chemical modifications of the oligonucleotide of any oligonucleotide listed in Table 1A.

In some embodiments, the pattern of backbone linkages, the pattern of backbone chiral centers, and the pattern of chemical modifications of the oligonucleotide are the pattern of backbone linkages, the pattern of backbone chiral centers, and/or the pattern of chemical modifications of the oligonucleotide of an oligonucleotide listed in Table 1A the base sequence of Y¹ comprises or is the base sequence of any APOC3 oligonucleotide listed in Table 1A, or the base sequence of Y¹ comprises 15 contiguous bases of the sequence of any APOC3 oligonucleotide listed in Table 1A.

In some embodiments, the oligonucleotide is capable of mediating a decrease in the expression, level and/or activity of an APOC3 target gene or a gene product thereof.

In some embodiments, the oligonucleotide is capable of mediating a decrease in the expression, level and/or activity of an APOC3 target gene or a gene product thereof via a mechanism mediated by RNaseH, steric hindrance and/or RNA interference.

In some embodiments:

each T¹ is independently absent or is alkylene, wherein one or more —CH₂— groups of the alkylene, may each independently be replaced with a heteroatom group independently selected from —O—, and —N(R⁴⁹)— wherein the heteroatom groups are separated by at least 2 carbon atoms;

each Q¹ is independently absent or is —C(O), —C(O)—NR⁴⁹, —NR⁴⁹—C(O), or a heteroatom group selected from —O—, and —NR⁴⁹, wherein at least two carbon atoms separate the heteroatom groups —O— and —NR⁴⁹ from any other heteroatom group;

each R⁴⁹ is independently —H, —(C₁-C₆)alkyl, or —(C₃-C₆)cycloalkyl wherein the alkyl and cycloalkyl may be substituted with halo atoms;

each n is independently 0, 1, 2, 3 or 4; wherein if n is greater than 0, each T¹ and each Q¹ of each (T¹-Q¹-T¹-Q¹) is independently selected;

each R⁴⁶ is —CH₂—OH;

each R⁴⁷ is —N(R⁴⁸)—C(O)—R⁴⁸; and

each R⁴⁸ is independently —H, or —(C₁-C₅)alkyl.

In some embodiments, the present disclosure pertains to: a compound having the Formula O2:

Y¹-L²-(Z¹¹)_{za} O2

or a pharmaceutically acceptable salt thereof wherein Y¹ is an oligonucleotide targets APOC3;

za is 1, 2, or 3;

L² is a linking group; and

Z¹¹ is a compound of Formula (B), wherein connection site with L² is indicated:

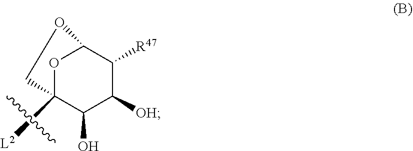

each R⁴⁷ is independently —OH, —N₃, —N(R⁴⁸)₂, —N(R⁴⁸)—C(O)—R⁴⁸, —N(R⁴⁸)—C(O)—N(R⁴⁸)₂, —N(R⁴⁸)—C(O)—OR⁴⁸, —N(R⁴⁸)—S(O)₂—R⁴⁸, tetrazole, or triazole, wherein the tetrazole and triazole are) optionally substituted with R⁴⁸;

each R⁴⁸ is independently —H, —(C₁-C₅)alkyl, halo-substituted —(C₁-C₅)alkyl, halo substituted —(C₃-C₆)cycloalkyl, —(C₁-C₅)alkenyl, —(C₁-C₅)alkynyl, halo substituted —(C₁-C₅)alkenyl, halo substituted —(C₁-C₅)alkynyl, or —(C₃-C₆)cycloalkyl, wherein a —CH₂— group of the alkyl or cycloalkyl may each be independently replaced with a heteroatom group selected from —O—, —S—, and —N(R⁵²)— and —CH₃ of the alkyl may each be independently replaced with a heteroatom group selected from —N(R⁵²)₂, —OR⁵², and —S(R⁵²) wherein the heteroatom groups are separated by at least 2 carbon atoms;

each R⁴⁹ is independently —H, —(C₁-C₂₀)alkyl, or —(C₃-C₆)cycloalkyl wherein one to six —CH₂— groups of the alkyl or cycloalkyl separated by at least two carbon atoms may be replaced with —O—, —S—, or —N(R^{49a})—, and —CH₃ of the alkyl may be replaced with a heteroatom group selected from —N(R^{49a})2, —OR^{49a}, and —S(R^{49a}) wherein the heteroatom groups are separated by at least 2 carbon atoms; and wherein the alkyl and cycloalkyl may be substituted with halo atoms and wherein each R^{49a} is independently —H, —(C₁-C₆)alkyl, or —(C₃-C₆)cycloalkyl;

each R⁵² is independently —H, —(C₁-C₂₀)alkyl, —(C₁-C₂₀)alkenyl, —(C₁-C₂₀)alkynyl, or —(C₃-C₆)cycloalkyl wherein one to six —CH₂— groups of the alkyl or cycloalkyl separated by at least two carbon atoms may each be independently replaced with a heteroatom independently selected from —O—, —S—, or —N(R⁴⁹)—, and —CH₃ of the alkyl may each be independently replaced with a heteroatom group selected from —N(R⁴⁹)₂, —OR⁴⁹, and —S(R⁴⁹) wherein the heteroatom groups are separated by at least 2 carbon atoms; and wherein the alkyl, alkenyl, alkynyl, and cycloalkyl may be substituted with halo atoms.

In some embodiments, L² is a compound of Formula L1, L2, L3, L4, L5, L6, L7, L8, L9, L10, L11, L12, L13 or L14, wherein connection sites with Y¹ and Z¹¹ are indicated:

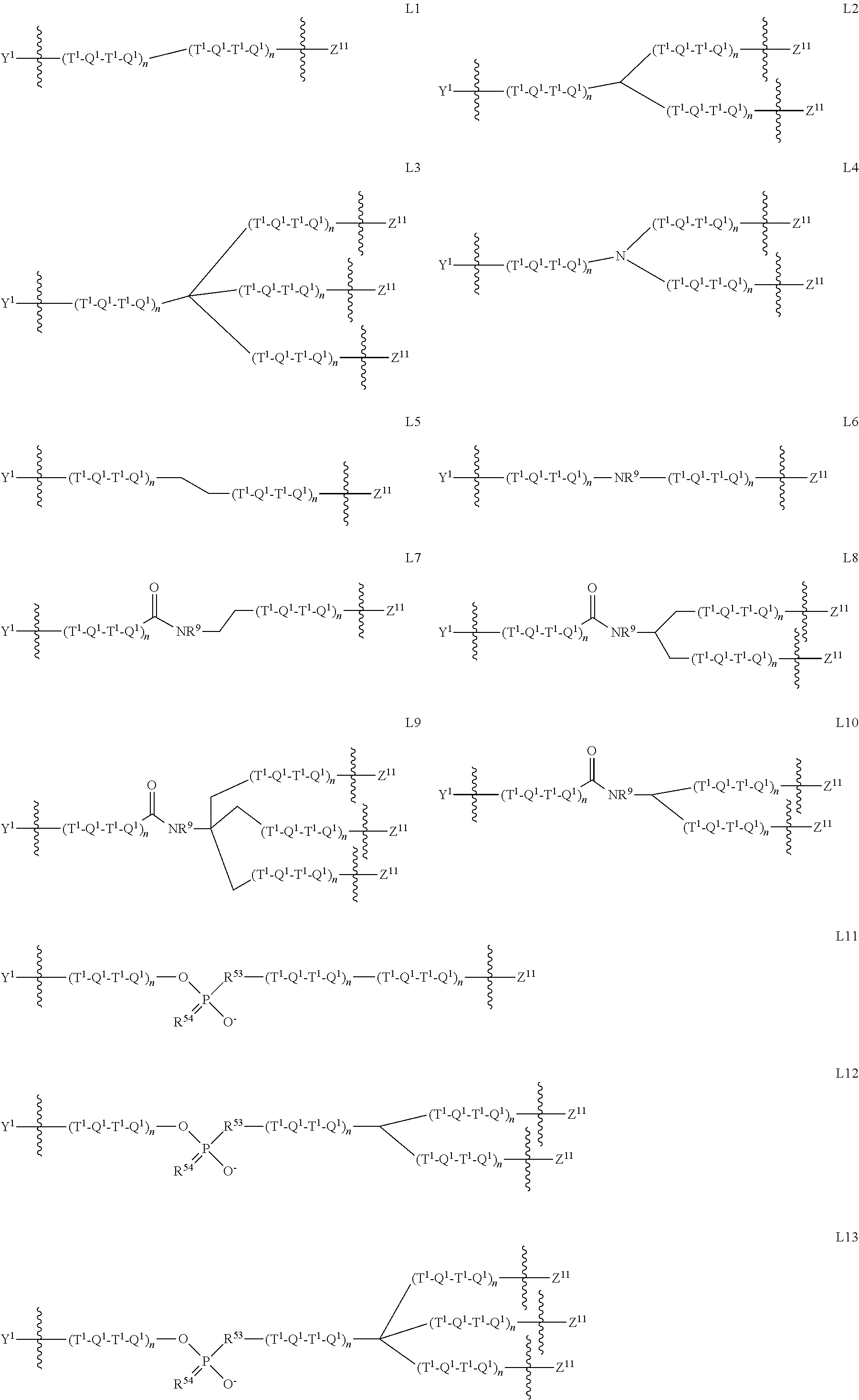

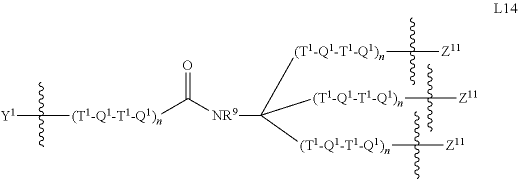

wherein each T¹ is independently absent or is alkylene, alkenylene, or alkynylene, wherein one or more —CH₂— groups of the alkylene, alkenylene, or alkynylene may each independently be replaced with a heteroatom group independently selected from —O—, —S—, and —N(R⁴⁹)— wherein the heteroatom groups are separated by at least 2 carbon atoms;

each Q¹ is independently absent or is —C(O)—, —C(O)—NR⁴⁹—, —O—C(O)—NR⁴⁹—, —NR⁴⁹—C(O)—O—, —CH₂—, —NR⁴⁹C(O)NR⁴⁹—, a bivalent heteroaryl group, or a heteroatom group selected from —O—, —S—, —S—S—, —S(O)—, —S(O)2-, and —NR⁴⁹—, wherein at least two carbon atoms separate the heteroatom groups —O—, —S—, —S—S—, —S(O)—, —S(O)2- and —NR⁴⁹— from any other heteroatom group, or a structure of the formula:

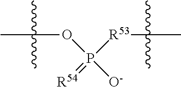

wherein R⁵³ is —O or —NH, and R⁵⁴ is —O or —S;

each R⁴⁹ is independently —H, —(C₁-C₂₀)alkyl, or —(C₃-C₆)cycloalkyl wherein one to six —CH₂— groups of the alkyl or cycloalkyl separated by at least two carbon atoms may be replaced with —O—, —S—, or —N(R^{49a})—, and —CH₃ of the alkyl may be replaced with a heteroatom group selected from —N(R^{49a})2, —OR^{49a}, and —S(R^{49a}) wherein the heteroatom groups are separated by at least 2 carbon atoms; and wherein the alkyl and cycloalkyl may be substituted with halo atoms; and wherein each R^{49a} is independently —H, —(C₁-C₆)alkyl, or —(C₃-C₆)cycloalkyl;

R⁵³ is —O or —NH;

R⁵⁴ is —O or —S; and

each n is independently 0, 1, 2, 3, 4, 5, 6, 7, 8, 9, 10,11,12,13,14, 15, 16, 17, 18, 19, 20, 21, 22, 23, 24, 25, 26, 27, 28, 29, 30, 31, 32, 33, 34, 35, 36, 37, 38, 39, or 40; wherein if n is greater than 0, each T¹ and each Q¹ of each (T¹-Q¹-T¹-Q¹) is independently selected.

In some embodiments, Y¹ comprises at least 15 bases.

In some embodiments, the base sequence of Y¹ comprises or is the base sequence of any APOC3 oligonucleotide listed in Table 1A, or the base sequence of Y¹ comprises 15 contiguous bases of the sequence of any APOC3 oligonucleotide listed in Table 1A.

In some embodiments, Y¹ comprises at least 1 phosphodiester internucleotidic linkage.

In some embodiments, Y¹ comprises at least 1 chirally controlled modified internucleotidic linkage.

In some embodiments, Y¹ comprises at least 1 chirally controlled modified internucleotidic linkage which is a chirally controlled phosphorothioate.

In some embodiments, Y¹ comprises at least 1 chirally controlled modified internucleotidic linkage which is a chirally controlled phosphorothioate in the Sp configuration.

In some embodiments, Y¹ comprises at least 1 chirally controlled modified internucleotidic linkage which is a chirally controlled phosphorothioate in the Rp configuration.

In some embodiments, Y¹, wherein the chirally controlled modified internucleotidic linkage or chirally controlled phosphorothioate comprises a phosphorus chiral center which has a diastereopurity of at least 70% within the composition.

In some embodiments, Y¹, wherein the chirally controlled modified internucleotidic linkage or chirally controlled phosphorothioate comprises a phosphorus chiral center which has a diastereopurity of at least 70%, 75%, 80%, 85%, 90%, 91%, 92%, 93%, 94%, 95%, 96%, 97%, 98%, 99% or 99.5%.

In some embodiments, Y¹ comprises at least 1 sugar modification.

In some embodiments, Y¹ comprises at least 1 base modification.

In some embodiments, the pattern of backbone linkages of the oligonucleotide is the pattern of backbone linkages of any oligonucleotide listed in Table 1A.

In some embodiments, the pattern of backbone chiral centers of the oligonucleotide is the pattern of backbone chiral centers of any oligonucleotide listed in Table 1A.

In some embodiments, the pattern of chemical modifications of the oligonucleotide is the pattern of chemical modifications of any oligonucleotide listed in Table 1A.

In some embodiments, the pattern of backbone linkages, the pattern of backbone chiral centers, and/or the pattern of chemical modifications of the oligonucleotide are the pattern of backbone linkages, the pattern of backbone chiral centers, and/or the pattern of chemical modifications of the oligonucleotide of any oligonucleotide listed in Table 1A.

In some embodiments, the pattern of backbone linkages, the pattern of backbone chiral centers, and the pattern of chemical modifications of the oligonucleotide are the pattern of backbone linkages, the pattern of backbone chiral centers, and/or the pattern of chemical modifications of the oligonucleotide of any oligonucleotide listed in Table 1A.

In some embodiments, the pattern of backbone linkages, the pattern of backbone chiral centers, and the pattern of chemical modifications of the oligonucleotide are the pattern of backbone linkages, the pattern of backbone chiral centers, and/or the pattern of chemical modifications of Y¹ is that of an oligonucleotide listed in Table 1A and the base sequence of Y′ comprises or is the base sequence of any APOC3 oligonucleotide listed in Table 1A, or the base sequence of Y′ comprises 15 contiguous bases of the sequence of any APOC3 oligonucleotide listed in Table 1A.

In some embodiments, the oligonucleotide is capable of mediating a decrease in the expression, level and/or activity of an APOC3 target gene or a gene product thereof.

In some embodiments, the oligonucleotide is capable of mediating a decrease in the expression, level and/or activity of an APOC3 target gene or a gene product thereof via a mechanism mediated by RNaseH, steric hindrance and/or RNA interference.

In some embodiments: each R⁴⁷ is —N(R⁴⁸)—C(O)—R⁴⁸; and each R⁴⁸ is independently —H, or —(C₁-C₅)alkyl.

In some embodiments:

each T¹ is independently absent or is alkylene, wherein

one or more —CH₂— groups of the alkylene, may each independently be replaced with a heteroatom group independently selected from —O—, and —N(R⁴⁹)— wherein the heteroatom groups are separated by at least 2 carbon atoms;

each Q¹ is independently absent or is C(O), C(O)—NR⁴⁹, NR⁴⁹—C(O), or a heteroatom group selected from O, and NR⁴⁹, wherein at least two carbon atoms separate the heteroatom groups O and NR⁴⁹ from any other heteroatom group;

each R⁴⁹ is independently —H, —(C₁-C₆)alkyl, or —(C₃-C₆)cycloalkyl wherein the alkyl and cycloalkyl may be substituted with halo atoms;

each n is independently 0, 1, 2, 3 or 4; wherein if n is greater than 0, each T¹ and each Q¹ of each (T¹-Q¹-T¹-Q¹) is independently selected.

In some embodiments, the present disclosure pertains to: a comprising a compound comprising: (a) an oligonucleotide capable of targeting APOC3; (b) a linking group; and (c) 1, 2, or 3 moieties independently selected from Z¹⁰ and Z¹¹; wherein the linking group links the oligonucleotide and the 1, 2 or 3 moieties, and wherein:
each Z¹⁰ is independently a compound of Formula Z12, Z13, Z14, Z15, Z16, Z17, Z18, Z19, Z20, or Z21, or a geometrical or position isomer thereof, wherein the connection site with L¹ is indicated:

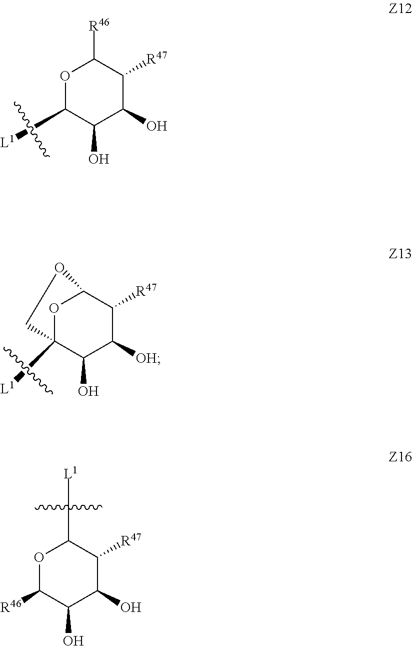

wherein each R⁴⁶ is independently —CN, —CH₂—CN, —C≡CH, —CH₂—N₃, —CH₂—NH₂, —CH₂—N(R⁵²)—S(O)₂—R⁵¹, —CH₂—CO₂H, —CO₂H, —CH₂—OH, —CH₂—SH, —CH═CH—R⁵¹, —CH₂—R⁵¹, —CH₂—S—R⁵¹, —CH₂—N(R⁵²)—R⁵¹, —CH₂—N(R⁵²)—C(O)—R⁵¹, —CH₂—N(R⁵²)—C(O)—O—R⁵¹, —CH₂—N(R⁵²)—C(O)—N(R⁵²)—R⁵¹, —CH₂—O—R⁵¹, —CH₂—O—C(O)—R ⁵¹, —CH₂—O—C(O)—N(R⁵²)—R⁵¹, —CH₂—O—C(O)—O—R⁵¹, —CH₂—S(O)—R⁵¹, —CH₂—S(O)₂—R⁵¹, —CH₂—S(O)₂—N(R⁵²)—R⁵¹, —C(O)—NH₂, —C(O)—O—R⁵¹, —C(O)—N(R⁵²)—R⁵¹, or aryl or heteroaryl, wherein the aryl or heteroaryl is optionally substituted with R5′

each R⁴⁷ is independently —OH, —N₃, —N(R⁴⁸)₂, —N(R⁴⁸)—C(O)—R⁴⁸, —N(R⁴⁸)—C(O)—N(R⁴⁸)₂, —N(R⁴⁸)—C(O)—OR⁴⁸, —N(R⁴⁸)—S(O)2-R⁴⁸, tetrazole, or triazole, wherein the tetrazole and triazole are optionally substituted with R⁴⁸;

each R⁴⁸ is independently —H, —(C₁-C₅)alkyl, halo-substituted (C₁-C₅)alkyl, halo substituted —(C₃-C₆)cycloalkyl, —(C₁-C₅)alkenyl, —(C₁-C₅)alkynyl, halo substituted —(C₁-C₅)alkenyl, halo substituted —(C₁-C₅)alkynyl, or —(C₃-C₆)cycloalkyl, wherein a —CH₂— group of the alkyl or cycloalkyl may each be independently replaced with a heteroatom group selected from —O—, —S—, and —N(R⁵²)— and —CH₃ of the alkyl may each be independently replaced with a heteroatom group selected from —N(R⁵²)₂, —OR⁵², and —S(R⁵²) wherein the heteroatom groups are separated by at least 2 carbon atoms;

each R⁵¹ is independently —H, —(C₃-C₂₀)cycloalkyl, —(C₁-C₆₀)alkenyl, —(C₁-C₆₀)alkynyl, or —(C₁-C₆₀)alkyl wherein one to six —CH₂— groups of the cycloalkyl or one to 20—CH₂— groups of the alkyl may each be independently replaced with heteroatoms independently selected from —O—, —S—, and —N(R⁴⁹)— wherein the heteroatoms are separated by at least two carbon atoms, and —CH₃ of the alkyl may each be independently replaced with a heteroatom group selected from —N(R⁴⁹)₂, —OR⁴⁹, and —S(R⁴⁹) wherein the heteroatom groups are separated by at least 2 carbon atoms, and wherein the alkyl, alkenyl, alkynyl, and cycloalkyl may be substituted with halo atoms; and

each R⁵² is independently —H, —(C₁-C₂₀)alkyl, —(C₁-C₂₀)alkenyl, —(C₁-C₂₀)alkynyl, or —(C₃-C₆)cycloalkyl wherein one to six —CH₂— groups of the alkyl or cycloalkyl separated by at least two carbon atoms may each be independently replaced with a heteroatom independently selected from —O—, —S—, or —N(R⁴⁹)—, and —CH₃ of the alkyl may each be independently replaced with a heteroatom group selected from —N(R⁴⁹)₂, —OR⁴⁹, and —S(R⁴⁹) wherein the heteroatom groups are separated by at least 2 carbon atoms; and wherein the alkyl, alkenyl, alkynyl, and cycloalkyl may be substituted with halo atoms;

each R⁴⁹ is independently —H, —(C₁-C₂₀)alkyl, or —(C₃-C₆)cycloalkyl wherein one to six —CH₂— groups of the alkyl or cycloalkyl separated by at least two carbon atoms may be replaced with —O—, —S—, or —N(R^{49a})—, and, —CH₃ of the alkyl may be replaced with a heteroatom group selected from —(N^{49a})₂, —OR^{49a}, and —S(R^{49a}) wherein the heteroatom groups are separated by at least 2 carbon atoms; and wherein the alkyl and cycloalkyl may be substituted with halo atoms and wherein each R^{49a} is independently —H, —(C₁-C₆)alkyl, or —(C₃-C₆)cycloalkyl;

each R^{49a} is independently —H, —(C₁-C₆)alkyl, or —(C₃-C₆)cycloalkyl;

and Z¹¹ is a compound of Formula (B), wherein connection site with L² is indicated:

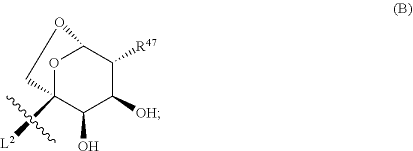

each R⁴⁷ is independently —OH, —N₃, —N(R⁴⁸)₂, —N(R⁴⁸)—C(O)—R⁴⁸, —N(R⁴⁸)—C(O)—N(R⁴⁸)₂, —N(R⁴⁸)—C(O)—OR⁴⁸, —N(R⁴⁸)—S(O)₂—R⁴⁸, tetrazole, or triazole, wherein the tetrazole and triazole are optionally substituted with R⁴⁸;

each R⁴⁸ is independently —H, halo-substituted —(C₁-C₅)alkyl, halo substituted —(C₃-C₆)cycloalkyl, —(C₁-C₅)alkenyl, —(C₁-C₅)alkynyl, halo substituted —(C₁-C₅)alkenyl, halo substituted —(C₁-C₅)alkynyl, or —(C₃-C₆)cycloalkyl, wherein a —CH₂— group of the alkyl or cycloalkyl may each be independently replaced with a heteroatom group selected from —O—, —S—, and —N(R⁵²)— and —CH₃ of the alkyl may each be independently replaced with a heteroatom group selected from —N(R⁵²)₂, —OR⁵², and —S(R⁵²) wherein the heteroatom groups are separated by at least 2 carbon atoms;

each R⁴⁹ is independently —H, —(C₁-C₂₀)alkyl, or —(C₃-C₆)cycloalkyl wherein one to six —CH₂— groups of the alkyl or cycloalkyl separated by at least two carbon atoms may be replaced with —O—, —S—, or —N(R^{49a})—, and —CH₃ of the alkyl may be replaced with a heteroatom group selected from —N(R^{49a})2, —OR′, and —S(R^{49a}) wherein the heteroatom groups are separated by at least 2 carbon atoms; and wherein the alkyl and cycloalkyl may be substituted with halo atoms and wherein each R^{49a} is independently —H, —(C₁-C₆)alkyl, or —(C₃-C₆)cycloalkyl;
46. each R⁵² is independently —H, —(C₁-C₂₀)alkyl, —(C₁-C₂₀)alkenyl, —(C₁-C₂₀)alkynyl, or —(C₃-C₆)cycloalkyl wherein one to six —CH₂— groups of the alkyl or cycloalkyl separated by at least two carbon atoms may each be independently replaced with a heteroatom independently selected from —O—, —S—, or —N(R⁴⁹)—, and —CH₃ of the alkyl may each be independently replaced with a heteroatom group selected from —N(R⁴⁹)₂, —OR⁴⁹, and —S(R⁴⁹) wherein the heteroatom groups are separated by at least 2 carbon atoms; and wherein the alkyl, alkenyl, alkynyl, and cycloalkyl may be substituted with halo atoms A chirally controlled APOC3 oligonucleotide composition comprising oligonucleotides of a particular oligonucleotide type characterized by:

a) a common base sequence and length, wherein the base sequence is complementary to an APOC3 target gene;

b) a common pattern of backbone linkages;

c) a common pattern of backbone chiral centers, wherein the common pattern of backbone chiral centers comprises at least one internucleotidic linkage comprising a chirally controlled chiral center;

which composition is chirally controlled in that it is enriched, relative to a substantially racemic preparation of oligonucleotides having the same common base sequence and length, for oligonucleotides of the particular oligonucleotide type; and

wherein the oligonucleotide composition is capable of decreasing the expression, level and/or activity of an APOC3 target gene or a gene product thereof.

In some embodiments, the oligonucleotides are capable of capable of decreasing the expression, level and/or activity of an APOC3 target gene or a gene product thereof via a mechanism mediated by RNaseH, steric hindrance and/or RNA interference.

In some embodiments, the present disclosure pertains to: a composition comprising a compound of any one of the preceding claims.

In some embodiments, the present disclosure pertains to: a composition comprising an APOC3 oligonucleotide which is a single-stranded RNAi agent, wherein the single-stranded RNAi agent is complementary or substantially complementary to an APOC3 target RNA sequence,

has a length of about 15 to about 49 nucleotides, and

is capable of directing target-specific RNA interference,

wherein the single-stranded RNAi agent comprises at least one non-natural base, sugar, and/or internucleotidic linkage, and

wherein the composition is capable of decreasing the expression, level and/or activity of an APOC3 target gene or a gene product thereof.

In some embodiments, the oligonucleotide or oligonucleotides further comprise a bridged bicyclic ketal.

In some embodiments, R^{CD} is

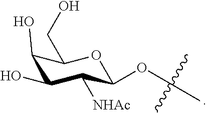

In some embodiments, R^{CD} is

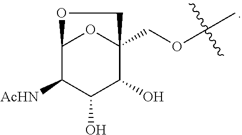 [SMILES: *OC[C@@]12CO[C@@H](O1)[C@H](NC(C)=O)[C@@H](O)[C@H]2O]

In some embodiments, R^{CD} is of such a structure that R^{CD}—H is

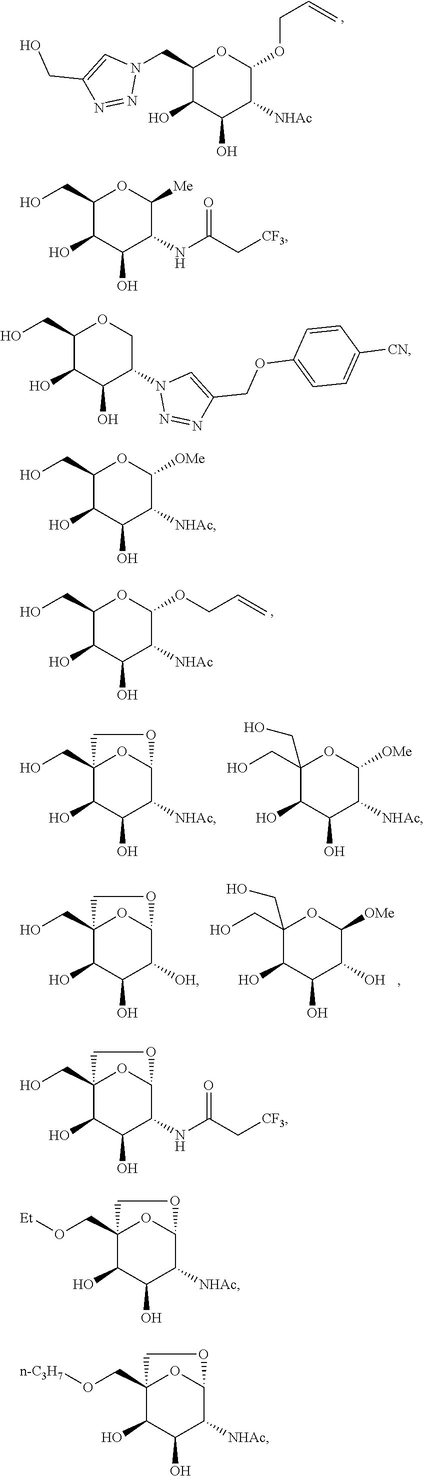

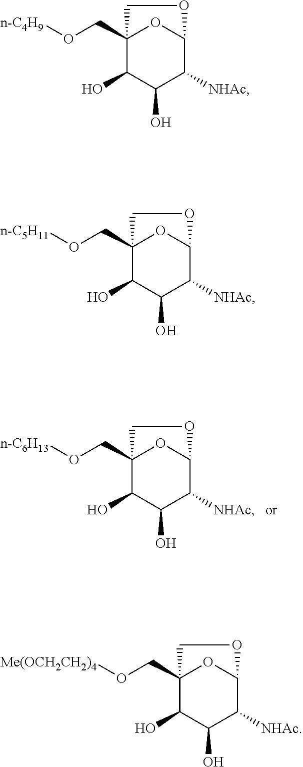

In some embodiments, R^{CD} is connected to the oligonucleotide or oligonucleotides through a linker.

In some embodiments, the linker is L^{M}.

In some embodiments, the linker has the structure of

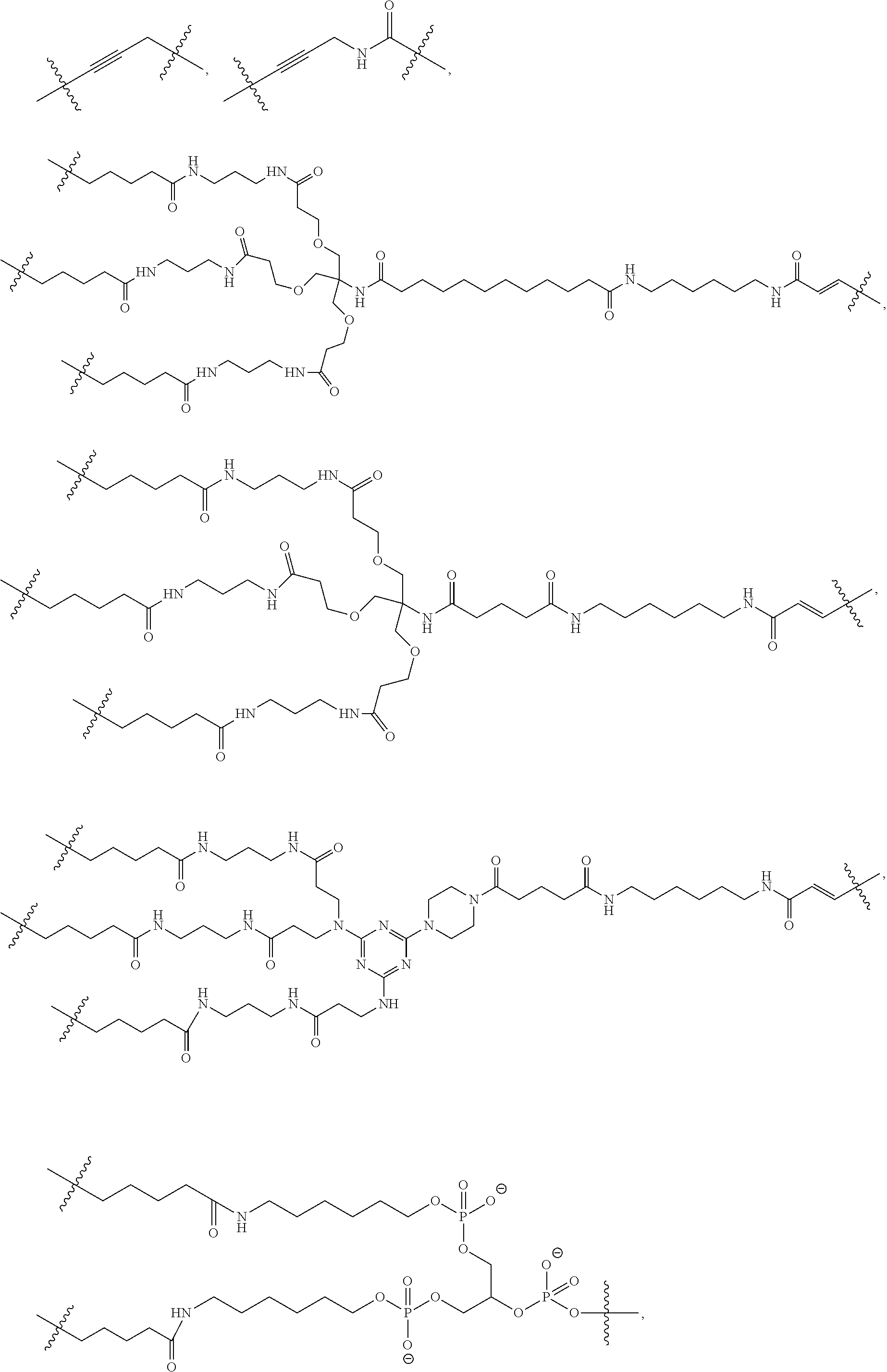 [SMILES: *C#CC* ; *C#CCNC(*)=O ; */C=C/C(=O)NCCCCCCNC(=O)CCCCCCCCCCC(=O)NC(COCCC(=O)NCCCNC(=O)CCCC*)(COCCC(=O)NCCCNC(=O)CCCC*)COCCC(=O)NCCCNC(=O)CCCC* ; */C=C/C(=O)NCCCCCCNC(=O)CCCC(=O)NC(COCCC(=O)NCCCNC(=O)CCCC*)(COCCC(=O)NCCCNC(=O)CCCC*)COCCC(=O)NCCCNC(=O)CCCC* ; */C=C/C(=O)NCCCCCCNC(=O)CCCC(=O)N1CCN(c2nc(NCCC(=O)NCCCNC(=O)CCCC*)nc(N(CCC(=O)NCCCNC(=O)CCCC*)CCC(=O)NCCCNC(=O)CCCC*)n2)CC1 ; *CCCCC(=O)NCCCCCCOP(=O)([O-])OCC(COP(=O)([O-])OCCCCCCNC(=O)CCCC*)OP(=O)([O-])O*]

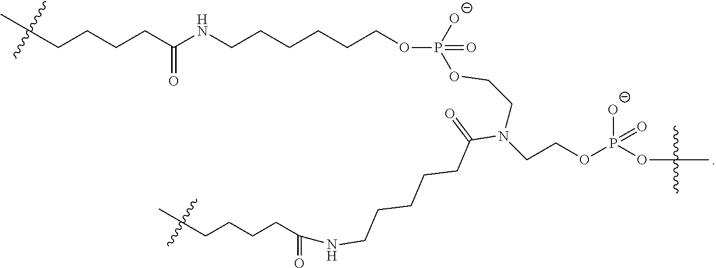 [SMILES: *CCCCC(=O)NCCCCCCOP(=O)([O-])OCCN(CCOP(=O)([O-])O*)C(=O)CCCCCNC(=O)CCCC*]

In some embodiments, R^{CD} is selected from:

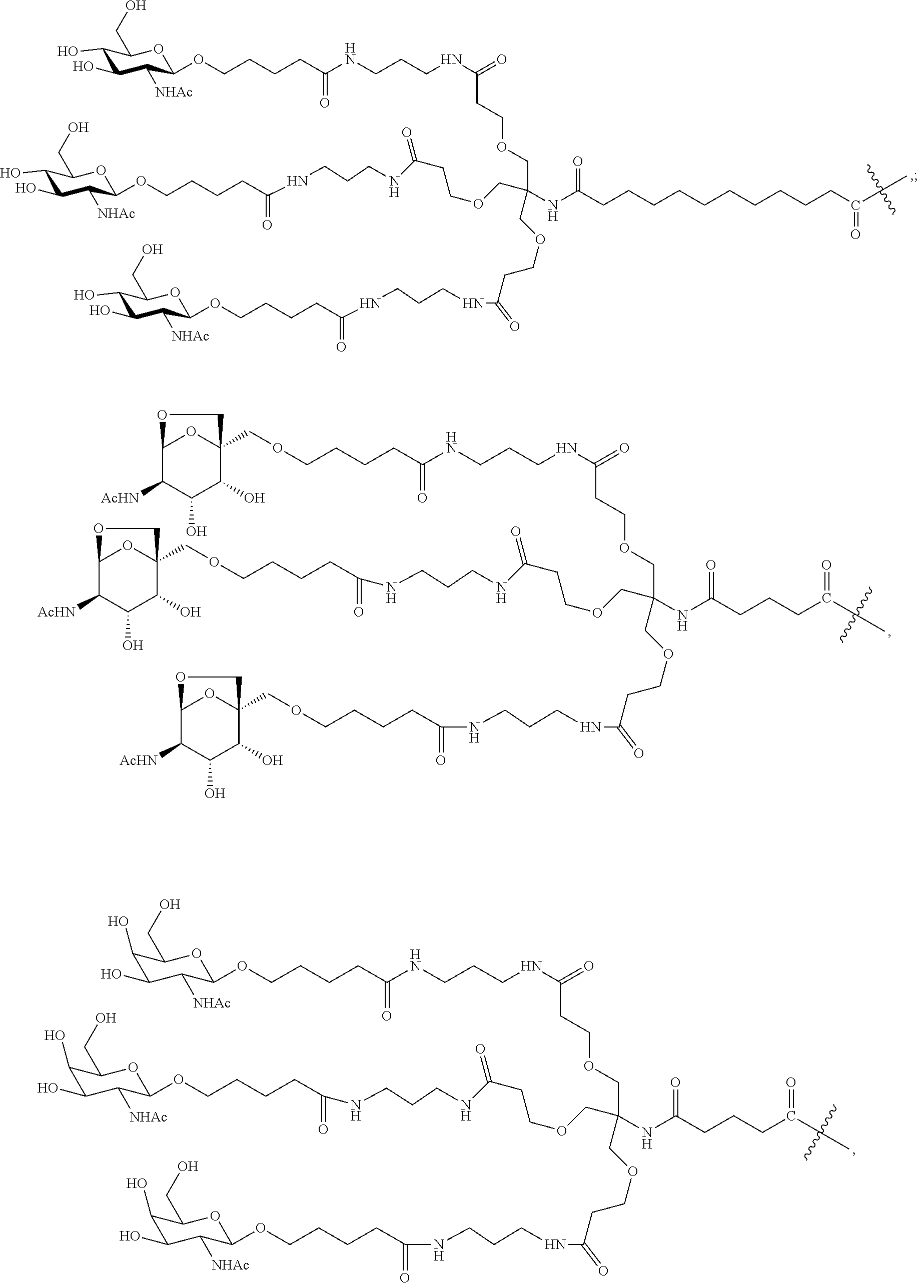

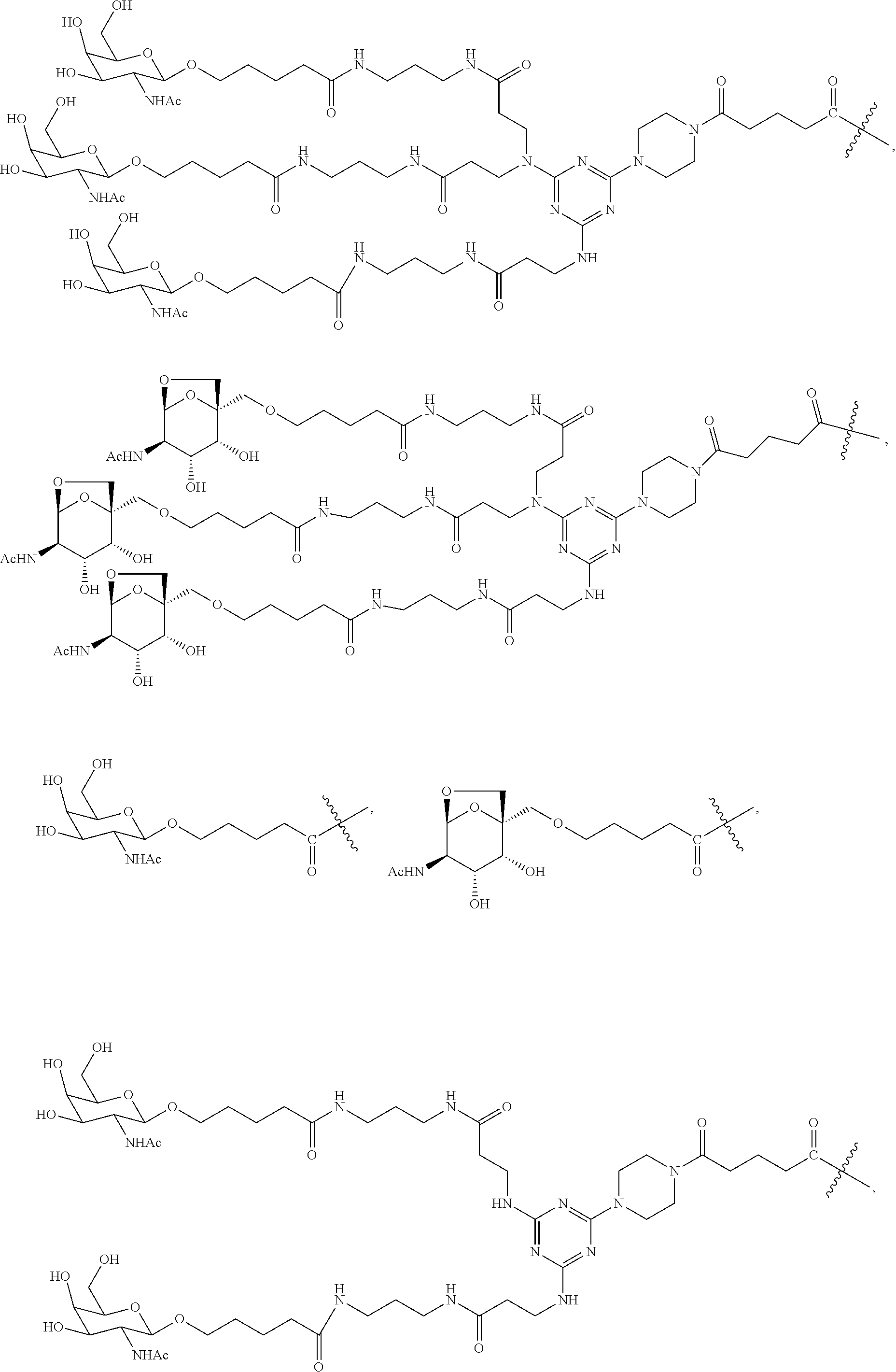

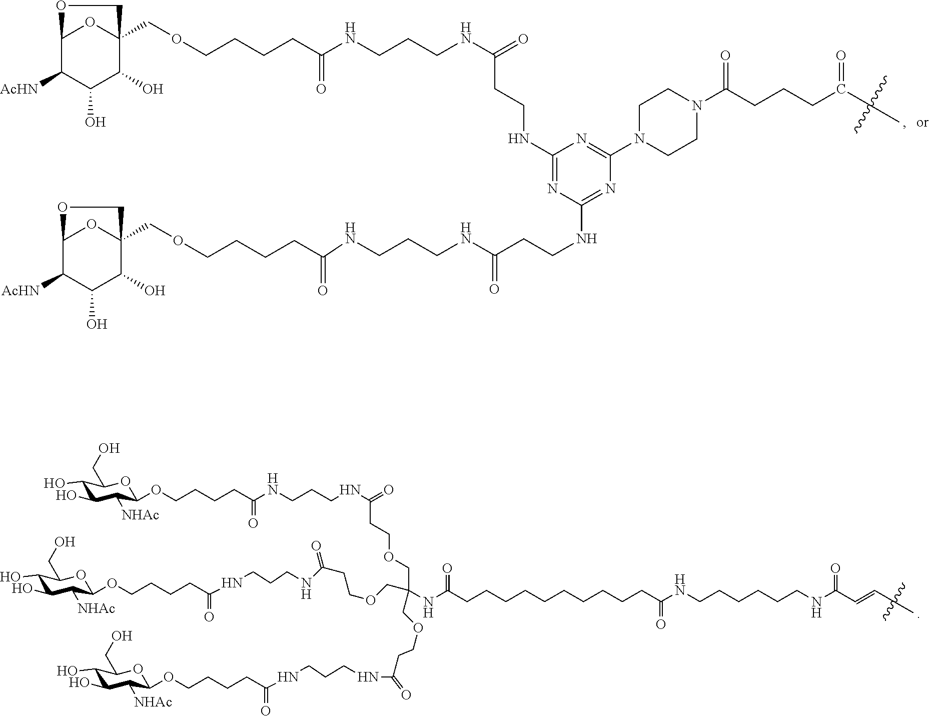

In some embodiments, the present disclosure pertains to: a pharmaceutical composition comprising a composition of any one of the preceding claims in a therapeutically effective amount, in admixture with at least one pharmaceutically acceptable excipient.

In some embodiments, the composition further comprises at least one additional pharmaceutical agent selected from the group consisting of an anti-inflammatory agent, an anti-diabetic agent, and a cholesterol/lipid modulating agent.

In some embodiments, said additional pharmaceutical agent is selected from the group consisting of an acetyl-CoA carboxylase- (ACC) inhibitor, a diacylglycerol O-acyltransferase 1 (DGAT-1) inhibitor, a diacylglycerol O-acyltransferase 2 (DGAT-2) inhibitor, monoacylglycerol O-acyltransferase inhibitors, a phosphodiesterase (PDE)-10 inhibitor, an AMPK activator, a sulfonylurea, a meglitinide, an α-amylase inhibitor, an α-glucoside hydrolase inhibitor, an α-glucosidase inhibitor, a PPARγ agonist, a PPAR α/γ agonist, a biguanide, a glucagon-like peptide 1 (GLP-1) modulator, liraglutide, albiglutide, exenatide, albiglutide, lixisenatide, dulaglutide, semaglutide, a protein tyrosine phosphatase-1B (PTP-1B) inhibitor, SIRT-1 activator, a dipeptidyl peptidase IV (DPP-IV) inhibitor, an insulin secreatagogue, a fatty acid oxidation inhibitor, an A2 antagonist, a c-jun amino-terminal kinase (JNK) inhibitor, glucokinase activators (GKa), insulin, an insulin mimetic, a glycogen phosphorylase inhibitor, a VPAC2 receptor agonist, SGLT2 inhibitors, a glucagon receptor modulator, GPR119 modulators, FGF21 derivatives or analogs, TGR5 receptor modulators, GPBAR1 receptor modulators, GPR40 agonists, GPR120 modulators, high affinity nicotinic acid receptor (HM74A) activators, SGLT1 inhibitors, inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g. PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostatin receptors, inhibitors or modulators of PDHK2 or PDHK4, inhibitors of MAP4K4, modulators of IL1 family including IL1beta, HMG-CoA reductase inhibitors, squalene synthetase inhibitors, fibrates, bile acid sequestrants, ACAT inhibitors, MTP inhibitors, lipooxygenase inhibitors, cholesterol absorption inhibitors, PCSK9 modulators, cholesteryl ester transfer protein inhibitors and modulators of RXRalpha.

In some embodiments, the composition further comprises at least one additional pharmaceutical agent selected from the group consisting of cysteamine or a pharmaceutically acceptable salt thereof, cystamine or a pharmaceutically acceptable salt thereof, an anti-oxidant compound, lecithin, vitamin B complex, a bile salt preparations, an antagonists of Cannabinoid-1 (CB1) receptor, an inverse agonists of Cannabinoid-1 (CB 1) receptor, a peroxisome proliferator-activated receptor) activity regulators, a benzothiazepine or benzothiepine compound, an RNA antisense construct to inhibit protein tyrosine phosphatase PTPRU, a heteroatom-linked substituted piperidine and derivatives thereof, an azacyclopentane derivative capable of inhibiting stearoyl-coenzyme alpha delta-9 desaturase, acylamide compound having secretagogue or inducer activity of adiponectin, a quaternary ammonium compound, Glatiramer acetate, pentraxin proteins, a HMG-CoA reductase inhibitor, n-acetyl cysteine, isoflavone compound, a macrolide antibiotic, a galectin inhibitor, an antibody, or any combination of thereof.

In some embodiments, the present disclosure pertains to: a method for the reduction of at least one point in severity of nonalcoholic fatty liver disease or nonalcoholic steatohepatitis grading scoring systems, reduction of the level of serum markers of nonalcoholic steatohepatitis activity, reduction of nonalcoholic steatohepatitis disease activity or reduction in the medical consequences of nonalcoholic steatohepatitis in humans comprising the step of administering to a human in need of such reduction a therapeutically effective amount of a composition of any one of the preceding claims to a patient in need thereof.

In some embodiments, the present disclosure pertains to: a method for treating fatty liver, nonalcoholic fatty liver disease, nonalcoholic steatohepatitis, nonalcoholic steatohepatitis with liver fibrosis, nonalcoholic steatohepatitis with cirrhosis, or nonalcoholic steatohepatitis with cirrhosis and hepatocellular carcinoma in humans comprising the step of administering to a human in need of such treatment a therapeutically effective amount of a composition of any one of the preceding claims to a patient in need thereof.

In some embodiments, the present disclosure pertains to: a method for treating hyperlipidemia, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, angina pectoris, thrombosis, atherosclerosis, transient ischemic attacks, stroke, vascular restenosis, hyperglycemia, hyperinsulinemia, hypertriglyceridemia, insulin resistance, impaired glucose metabolism, erectile dysfunction, skin and connective tissue disorders, foot ulcerations and ulcerative colitis, endothelial dysfunction and impaired vascular compliance, hyper apo B lipoproteinemia, Alzheimer's, schizophrenia, impaired cognition, inflammatory bowel disease, ulcerative colitis, Crohn's disease, and irritable bowel syndrome, non-alcoholic steatohepatitis (NASH), or non-alcoholic fatty liver disease (NAFLD), in humans comprising the step of administering to a human in need of such treatment a therapeutically effective amount of a composition of any one of the preceding claims to a patient in need thereof.

In some embodiments, the present disclosure pertains to: a method for treating fatty liver, nonalcoholic fatty liver disease, nonalcoholic steatohepatitis, nonalcoholic steatohepatitis with liver fibrosis, nonalcoholic steatohepatitis with cirrhosis, or nonalcoholic steatohepatitis with cirrhosis and hepatocellular carcinoma in humans comprising the step of administering to a human in need of such treatment a therapeutically effective amount of two separate pharmaceutical compositions comprising

a. a first composition of any one of the preceding claims; and

b. a second composition comprising at least one additional pharmaceutical agent selected from the group consisting of an anti-inflammatory agent, an anti-diabetic agent, and a cholesterol/lipid modulating agent and at least one pharmaceutically acceptable excipient.

In some embodiments, said first composition and said second composition are administered simultaneously.

In some embodiments, said first composition and said second composition are administered sequentially and in any order.

In some embodiments, the present disclosure pertains to: a method for reducing portal hypertension, hepatic protein synthetic capability, hyperbilirubinemia, or encephalopathy in humans comprising the step of administering to a human in need of such treatment a therapeutically effective amount of a composition of any one of the preceding claims to a patient in need thereof.

In some embodiments, the present disclosure pertains to: a method of decreasing the expression, activity and/or level of an APOC3 target gene or a gene product thereof in a cell, comprising the step of contacting the cell with a compound or composition of any one of the preceding claims.

In some embodiments, the present disclosure pertains to: a method of decreasing the expression, activity and/or level of an APOC3 target gene or a gene product thereof in a patient, comprising the step of contacting the cell with a compound or composition of any one of the preceding claims.

In some embodiments, a GalNAc, as the term is used herein, refers to a chemical entity which is structurally similar to a GalNAc and/or which performs at least one function of a GalNAc (e.g., binding to ASPGR).

In some embodiments, a 5′-end of a single-stranded RNAi agent comprises a GalNAc or a variant or derivative thereof.

A non-limiting example of a GalNAc moiety at the 5′-end of an APOC3 oligonucleotide or single-stranded RNAi agent (e.g., 5′ GalNAc moiety) is shown below, wherein the 5′ end structure is represented by:

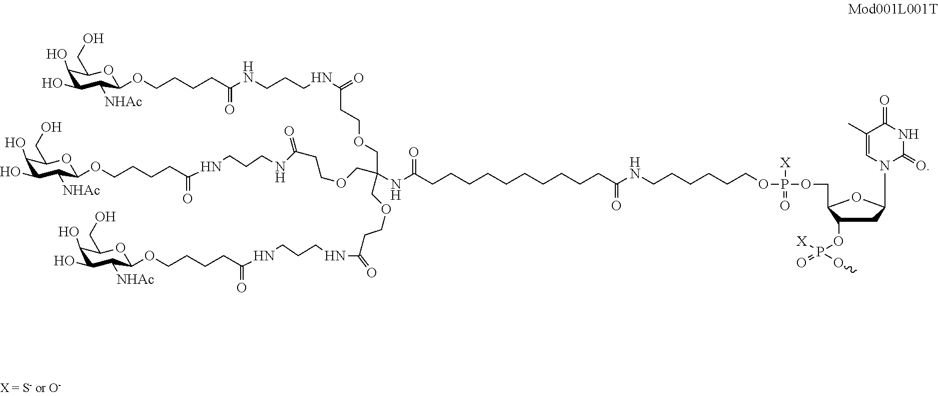

In some embodiments, a GalNAc moiety, e.g., a GalNAc or a variant or derivative thereof, is described in any of: Migawa et al. 2016 Bioorg. Med. Chem. Lett. 26: 2914-7; Ostergaard et al. 2015 Bioconjug. Chem. 26: 1451-1455; Prakash et al. 2014 Nucl. Acids Res. 42: 8796-8807; Prakash et al. 2016 J. Med. Chem. 59: 2718-33; Shemesh et al. 2016 Mol. Ther. Nucl. Acids 5: e319; St-Pierre et al. 2016 Bioorg. Med. Chem. 24: 2397-409; and/or Yu et al. 2016 Mol. Ther. Nucl. Acids 5: e317.

In some embodiments, a chemical moiety (e.g., additional component) conjugated to an APOC3 oligonucleotide binds to ASPGR.

In some embodiments, a chemical moiety (e.g., additional component) conjugated to an APOC3 oligonucleotide binds to ASPGR and comprises any of: Mod031, Mod034, Mod035, Mod036, Mod038, Mod039, Mod040, or Mod041.

In some embodiments, an additional component can be or comprise any of: Mod079, Mod080, Mod081, Mod082 or Mod083. In some embodiments, an additional component can be or comprise any of:

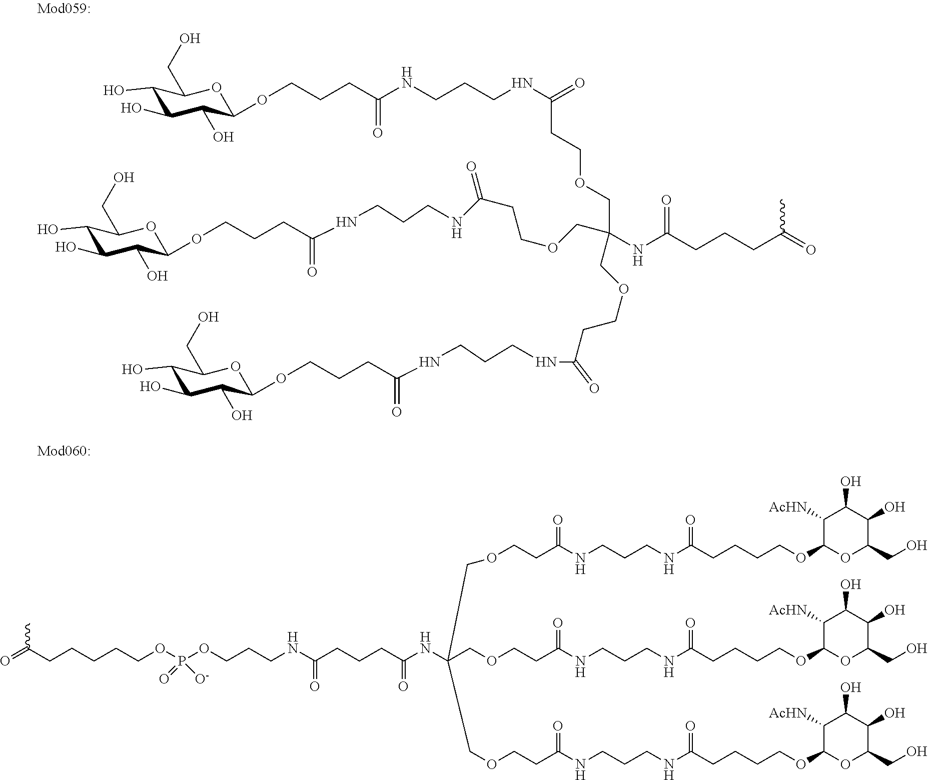

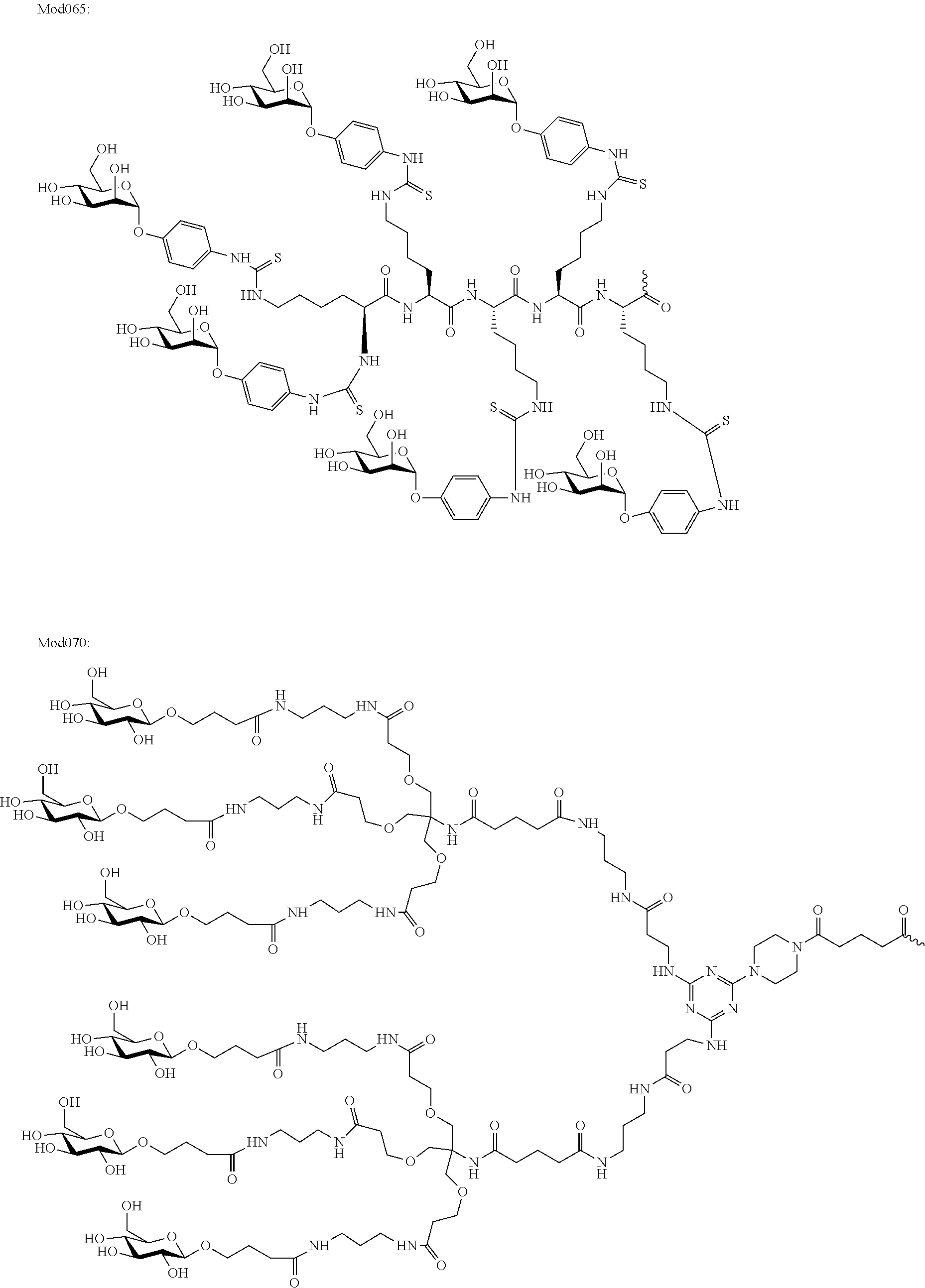

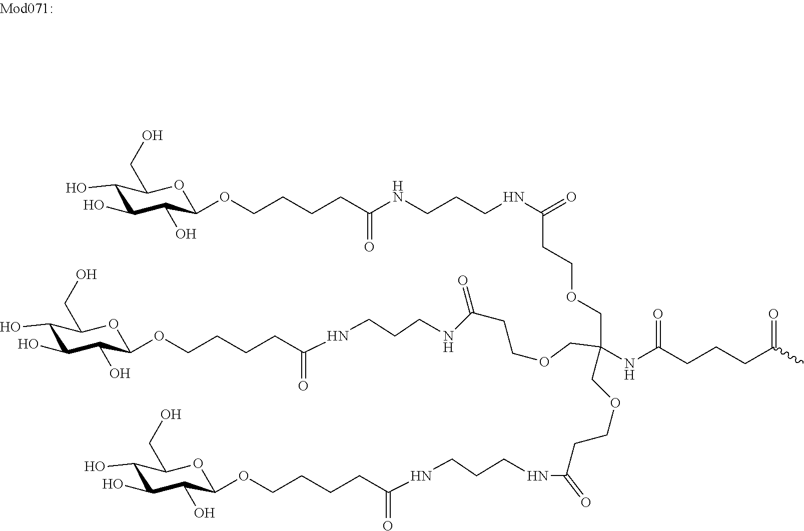

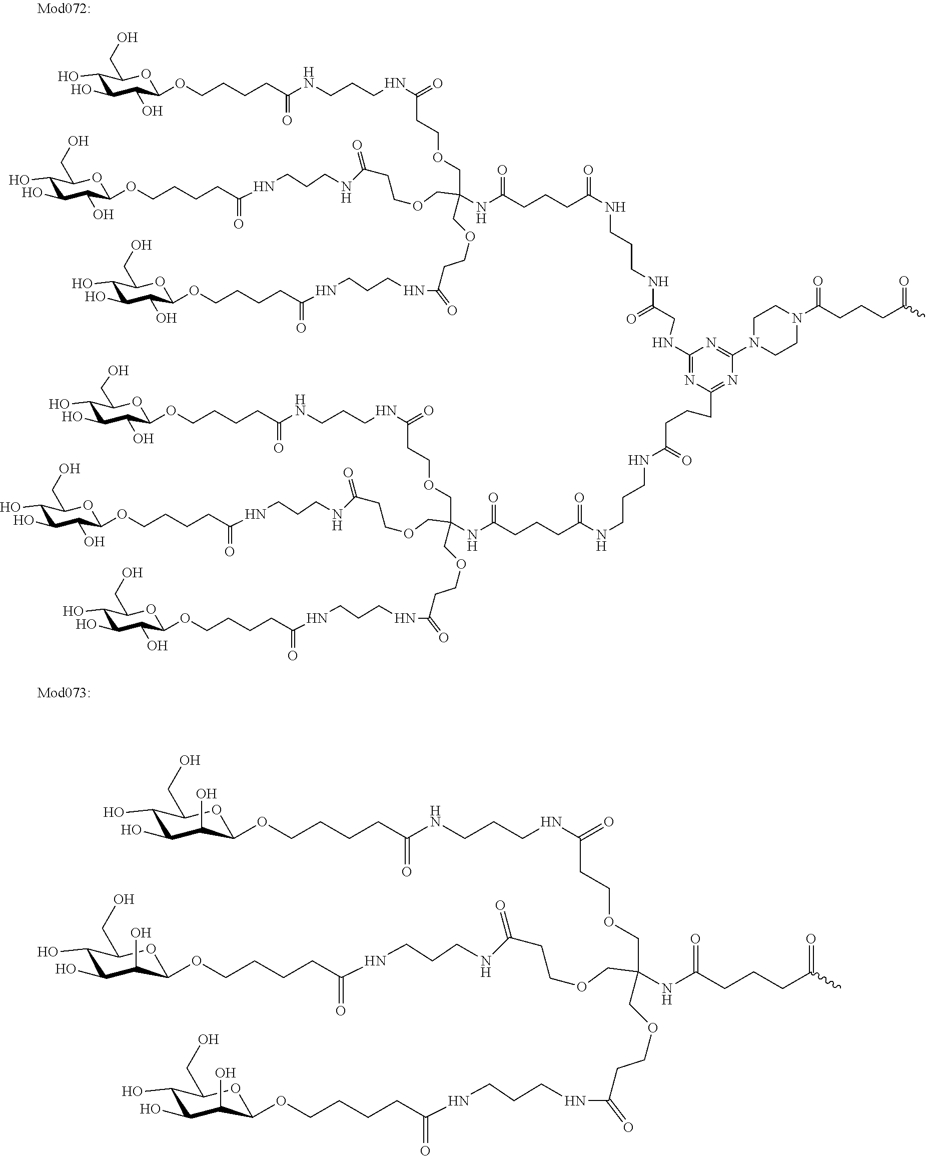

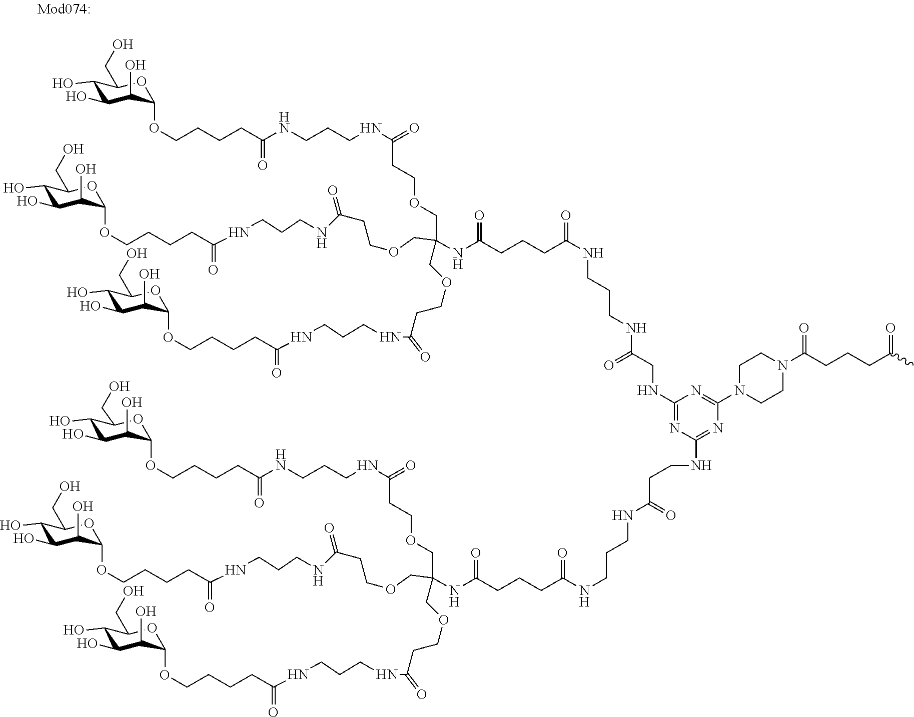

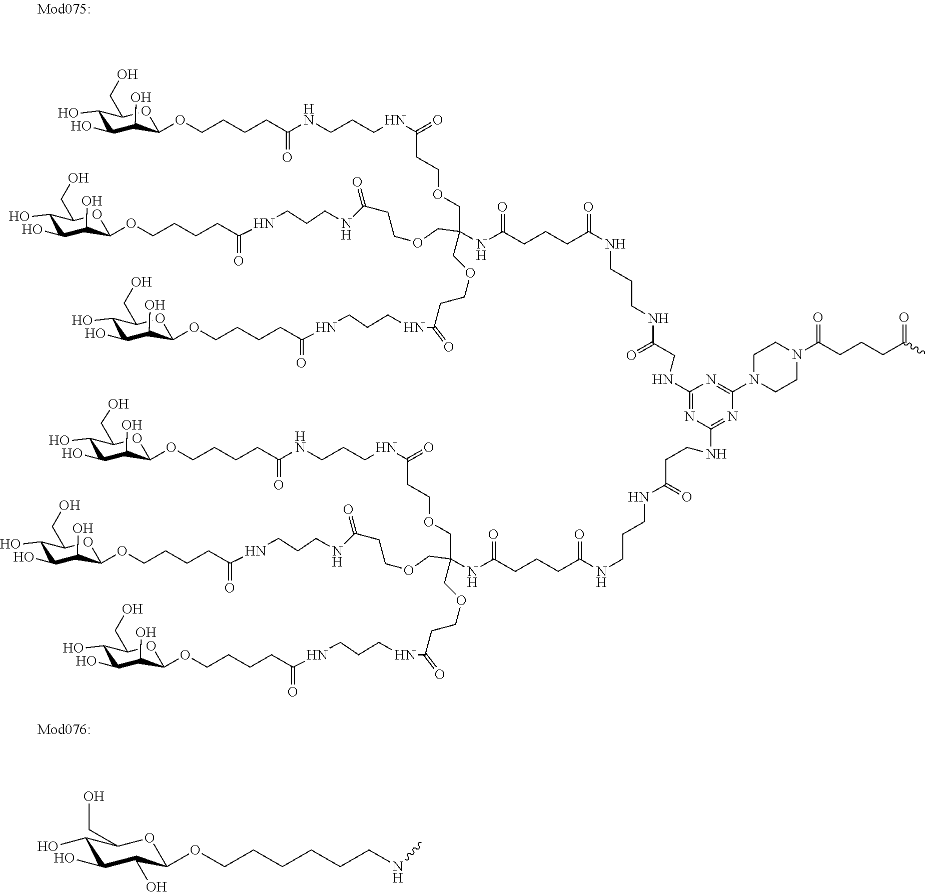

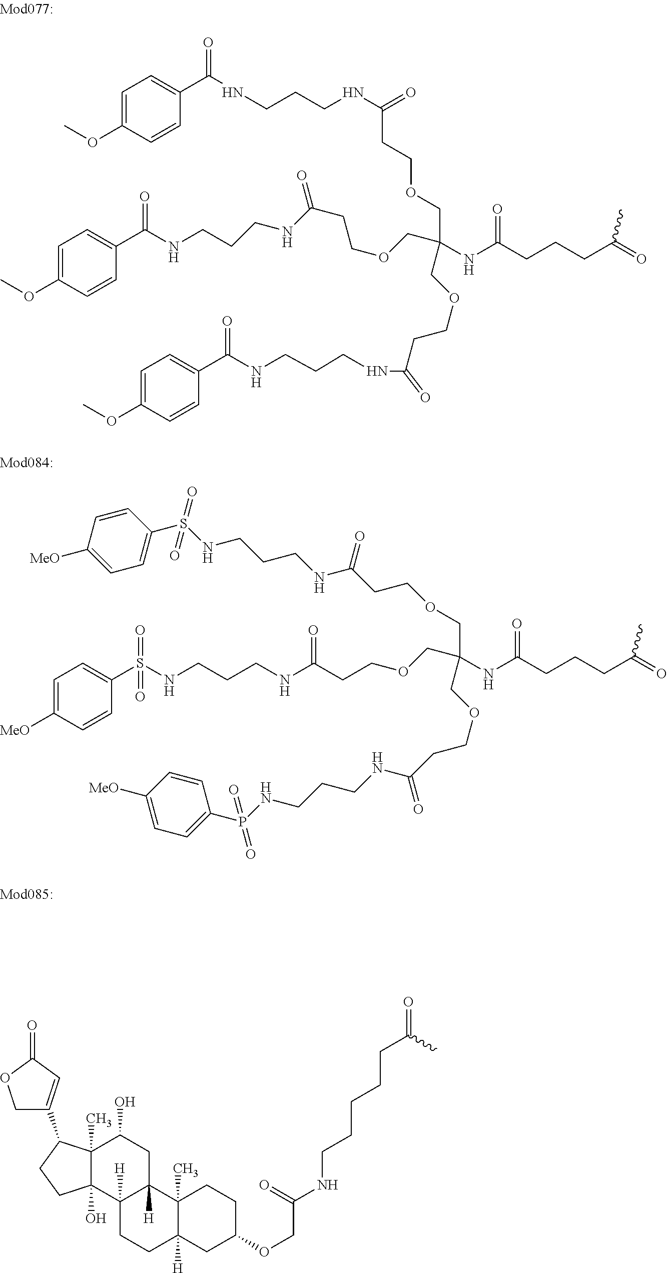

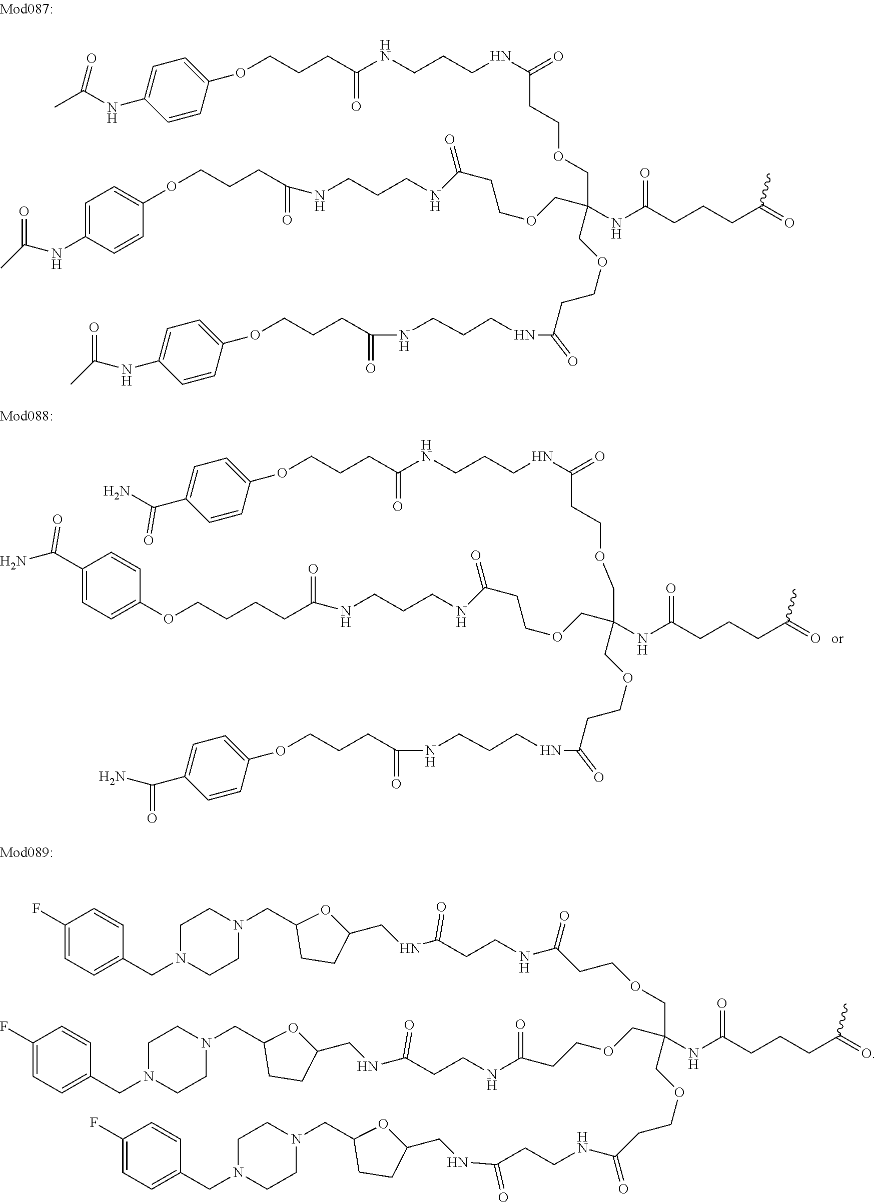

In some embodiments, an additional component can be or comprise:

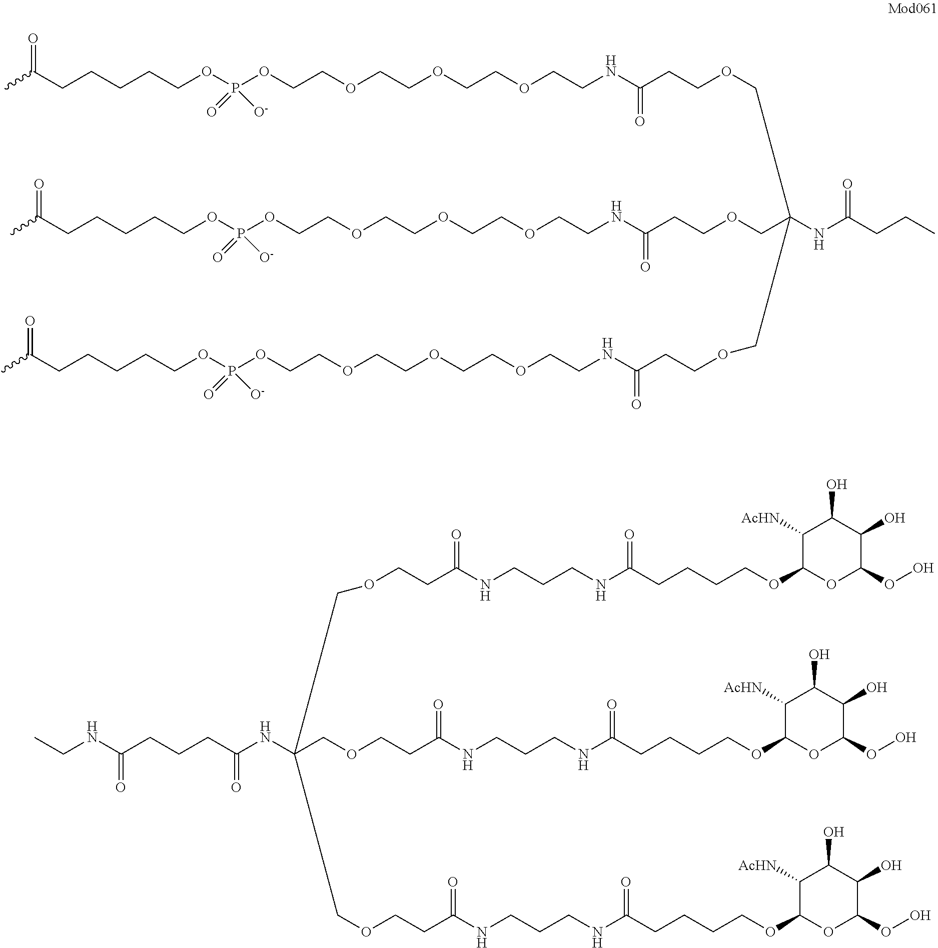

wherein Mod061 is conjugated to three identical or non-identical oligonucleotides.

5′ Nucleoside or 5′ Nucleotide of an APOC3 Oligonucleotide

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides can comprise any 5′-nucleoside or 5′-nucleotide described herein or known in the art.

In some embodiments, the 5′ nucleoside, e.g., the nucleoside at the 5′-end, of a single-stranded RNAi agent (e.g., in N1) can be any nucleoside, modified nucleoside or universal nucleoside known in the art.

In some embodiments, the 5′ nucleotide, e.g., the nucleoside at the 5′-end, of a single-stranded RNAi agent (e.g., in N1) can comprise a 2′ modification at the base.

In some embodiments, the nucleoside at the 5′-end of a single-stranded RNAi agent (e.g., in N1) can comprise a 2′-deoxy (DNA), 2′-F, 2′-OMe, or 2′-MOE, or an inverted nucleoside or nucleotide.

Non-limiting examples of ssRNAi agent formats in which the nucleoside at the 5′-end of the ssRNAi agent is a 2′-deoxy (DNA) include: Formats 1-5, 16-18, 22-29, 32-78, 84-93, 97, and 103-107 of FIG. 1.

Non-limiting examples of ssRNAi agent formats in which the nucleoside at the 5′-end of the ssRNAi agent is a 2′-F include: Formats 11-15, 19, 79-83, and 98-100 of FIG. 1.

Non-limiting examples of ssRNAi agent formats in which the nucleoside at the 5′-end of the ssRNAi agent is a 2′-OMe include: Formats 6-10, 20-21, 30-31, 94-96, and 101-102 of FIG. 1.

In some embodiments, the nucleobase at the 5′-end of a single-stranded RNAi agent (e.g., in N1) is T. In some embodiments, the nucleobase at the 5′-end of a single-stranded RNAi agent (e.g., in N1) is U. In some embodiments, the nucleobase at the 5′-end of a single-stranded RNAi agent (e.g., in N1) is A. In some embodiments, the nucleobase at the 5′-end of a single-stranded RNAi agent (e.g., in N1) is G. In some embodiments, the nucleobase at the 5′-end of a single-stranded RNAi agent (e.g., in N1) is C.

In some embodiments, a provided single-stranded RNAi agent has a 5′ mismatch, wherein the nucleobase at the 5′-end of the single-stranded RNAi agent (position N1) has a mismatch from the corresponding position of the target transcript. As has been reported in the art, complementarity between the 5′ nucleotide moiety and the corresponding position of the target transcript is not required for efficacious double-stranded siRNAs. Various example single-stranded RNAi agents described herein also have a 5′ mismatch and are still capable of directing RNA interference. Efficacious ssRNAi agents have been constructed which have a mismatch with the sequence of the target mRNA at the 5′ position (N1). Efficacious ssRNAi agents have been constructed which have a mismatch with the sequence of the target mRNA at the 5′ position and the N1 position of the ssRNAi is T. In some embodiments, a provided single-stranded RNAi agent has a 5′ mismatch at N1, wherein the nucleobase in N1 is T.

In some embodiments, the nucleoside at the 5′ position is a LNA.

In some embodiments, the nucleoside at the 5′ position is a 5′-H (deoxy). Efficacious ssRNAi agents have been constructed wherein the nucleoside at the 5′ position is a 5′-H (deoxy). In some embodiments, the nucleoside at the 5′ position is deoxy T, A, G, or C. In some embodiments, the nucleoside at the 5′ position is deoxy T. In some embodiments, the nucleoside at the 5′ position is deoxy A. In some embodiments, the nucleoside at the 5′ position is a 2′-F. Efficacious ssRNAi agents have been constructed wherein the nucleoside at the 5′ position is a 2′-F. In some embodiments, the nucleoside at the 5′ position is a 2′-F A. In some embodiments, the nucleoside at the 5′ position is a 2′-F G. In some embodiments, the nucleoside at the 5′ position is a 2′-OMe. Efficacious ssRNAi agents have been constructed wherein the nucleoside at the 5′ position is a 2′-OMe. In some embodiments, the nucleoside at the 5′ position is a 2′-OMe U. In some embodiments, the nucleoside at the 5′ position is a 2′-OMe A. In some embodiments, the nucleoside at the 5′ position is a 2′-OMe C.

Seed Region of an APOC3 Oligonucleotide, Including a Single-Stranded RNAi Agent

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any seed region or portion or structural element thereof described herein or known in the art.

In some embodiments, a seed region of a provided single-stranded RNAi agent is a portion of the RNAi agent which is particularly important in binding of the RNAi agent to a transcript target. Lim et al. 2005 Nature 433: 769-773. In many cases, full complementarity between the seed region of the RNAi agent antisense strand and the mRNA target is reportedly required for high RNAi activity. For example, a single mismatch at position 6 in the seed region reportedly abolished double-stranded RNAi activity; Lim et al. 2005 Nature 433: 769-773. In contrast, dsRNAi antisense strands reportedly are more amenable to mismatches outside the seed region, e.g., at the 5′ position, in the post-seed region, and in the 3′-terminal dinucleotide.

In some embodiments, each nucleotide in the seed region is 2′-OMe.

In some embodiments, each nucleotide in the seed region is 2′-OMe, and each nucleotide in the post-seed region is 2′-OMe.

In some embodiments, one nucleotide in the seed region is 2′-F and each other nucleotide in the seed region is 2′-OMe.

In some embodiments, one nucleotide in the seed region is 2′-F and each other nucleotide in the seed region is 2′-OMe, and one nucleotide in the post-seed region is 2′-F and each other nucleotide in the post-seed region is 2′-OMe.

In some embodiments, the nucleotide at position 2 (counting from the 5′-end) is 2′-F and each other nucleotide in the seed region is 2′-OMe, and one nucleotide in the post-seed region is 2′-F and each other nucleotide in the post-seed region is 2′-OMe.

In some embodiments, the nucleotide at position 2 (counting from the 5′-end) is 2′-F and each other nucleotide in the seed region is 2′-OMe, and the nucleotide at position 14 (counting from the 5′-end) is 2′-F and each other nucleotide in the post-seed region is 2′-OMe.

In some embodiments, a provided single-stranded RNAi agent comprises a seed region in which any number of N can be 2′-deoxy, 2′-F, 2′-OMe and/or 2′-OH, and/or have any other modification at the 2′ position of the sugar.

Various non-limiting examples of seed regions of single-stranded RNAi agents are presented in Table 1A, the Figures, and elsewhere herein. In some embodiments, a provided single-stranded RNAi agent comprises a seed region, in which any one or more nucleotides in the seed region are independently 2′-F. In some embodiments, a provided single-stranded RNAi agent comprises a seed region, in which any 2 or more nucleotides in the seed region are independently 2′-F. In some embodiments, a provided single-stranded RNAi agent comprises a seed region, in which any 3 or more nucleotides in the seed region are independently 2′-F. In some embodiments, a provided single-stranded RNAi agent comprises a seed region, in which any 4 or more nucleotides in the seed region are independently 2′-F. In some embodiments, a provided single-stranded RNAi agent comprises a seed region, in which any 5 or more nucleotides in the seed region are independently 2′-F. In some embodiments, a provided single-stranded RNAi agent comprises a seed region, in which any 6 or more nucleotides in the seed region are independently 2′-F.

In some embodiments, a provided single-stranded RNAi agent comprises a seed region, in which any one or more nucleotides in the seed region are independently 2′-OMe. In some embodiments, a provided single-stranded RNAi agent comprises a seed region, in which any 2 or more nucleotides in the seed region are independently 2′-OMe. In some embodiments, a provided single-stranded RNAi agent comprises a seed region, in which any 3 or more nucleotides in the seed region are independently 2′-OMe. In some embodiments, a provided single-stranded RNAi agent comprises a seed region, in which any 4 or more nucleotides in the seed region are independently 2′-OMe. In some embodiments, a provided single-stranded RNAi agent comprises a seed region, in which any 5 or more nucleotides in the seed region are independently 2′-OMe. In some embodiments, a provided single-stranded RNAi agent comprises a seed region, in which any 6 or more nucleotides in the seed region are independently 2′-OMe. In some embodiments, a provided single-stranded RNAi agent comprises a seed region, in which 7 of N2 to N7 are independently 2′-OMe.

In some embodiments, a provided single-stranded RNAi agent comprises a seed region, in which any one or more nucleotides in the seed region are independently 2′-M0E. In some embodiments, a provided single-stranded RNAi agent comprises a seed region, in which any 2 or more nucleotides in the seed region are independently 2′-M0E. In some embodiments, a provided single-stranded RNAi agent comprises a seed region, in which any 3 or more nucleotides in the seed region are independently 2′-M0E. In some embodiments, a provided single-stranded RNAi agent comprises a seed region, in which any 4 or more nucleotides in the seed region are independently 2′-M0E. In some embodiments, a provided single-stranded RNAi agent comprises a seed region, in which any 5 or more nucleotides in the seed region are independently 2′-M0E. In some embodiments, a provided single-stranded RNAi agent comprises a seed region, in which any 6 or more nucleotides in the seed region are independently 2′-M0E. In some embodiments, a provided single-stranded RNAi agent comprises a seed region, in which 7 of N2 to N7 are independently 2′-M0E.

In some embodiments, a provided single-stranded RNAi agent comprises a seed region, in which any one or more nucleotides in the seed region are independently 2′-deoxy. In some embodiments, a provided single-stranded RNAi agent comprises a seed region, in which any 2 or more nucleotides in the seed region are independently 2′-deoxy. In some embodiments, a provided single-stranded RNAi agent comprises a seed region, in which any 3 or more nucleotides in the seed region are independently 2′-deoxy. In some embodiments, a provided single-stranded RNAi agent comprises a seed region, in which any 4 or more nucleotides in the seed region are independently 2′-deoxy. In some embodiments, a provided single-stranded RNAi agent comprises a seed region, in which any 5 or more nucleotides in the seed region are independently 2′-deoxy. In some embodiments, a provided single-stranded RNAi agent comprises a seed region, in which any 6 or more nucleotides in the seed region are independently 2′-deoxy. In some embodiments, a provided single-stranded RNAi agent comprises a seed region, in which 7 of N2 to N7 are independently 2′-deoxy.

In some embodiments, a provided single-stranded RNAi agent comprises a seed region, in which any one or more non-consecutive nucleotides in the seed region are independently 2′-F. In some embodiments, a provided single-stranded RNAi agent comprises a seed region, in which any 2 or more non-consecutive nucleotides in the seed region are independently 2′-F. In some embodiments, a provided single-stranded RNAi agent comprises a seed region, in which any 3 or more non-consecutive nucleotides in the seed region are independently 2′-F. In some embodiments, a provided single-stranded RNAi agent comprises a seed region, in which any 4 or more non-consecutive nucleotides in the seed region are independently 2′-F.

In some embodiments, a provided single-stranded RNAi agent comprises a seed region, in which any one or more non-consecutive nucleotides in the seed region are independently 2′-OMe. In some embodiments, a provided single-stranded RNAi agent comprises a seed region, in which any 2 or more non-consecutive nucleotides in the seed region are independently 2′-OMe. In some embodiments, a provided single-stranded RNAi agent comprises a seed region, in which any 3 or more non-consecutive nucleotides in the seed region are independently 2′-OMe. In some embodiments, a provided single-stranded RNAi agent comprises a seed region, in which any 4 or more non-consecutive nucleotides in the seed region are independently 2′-OMe.

In some embodiments, a provided single-stranded RNAi agent comprises a seed region, in which any one or more non-consecutive nucleotides in the seed region are independently 2′-M0E. In some embodiments, a provided single-stranded RNAi agent comprises a seed region, in which any 2 or more non-consecutive nucleotides in the seed region are independently 2′-M0E. In some embodiments, a provided single-stranded RNAi agent comprises a seed region, in which any 3 or more non-consecutive nucleotides in the seed region are independently 2′-M0E. In some embodiments, a provided single-stranded RNAi agent comprises a seed region, in which any 4 or more non-consecutive nucleotides in the seed region are independently 2′-M0E.

In some embodiments, a provided single-stranded RNAi agent comprises a seed region, in which any one or more non-consecutive nucleotides in the seed region are independently 2′-deoxy. In some embodiments, a provided single-stranded RNAi agent comprises a seed region, in which any 2 or more non-consecutive nucleotides in the seed region are independently 2′-deoxy. In some embodiments, a provided single-stranded RNAi agent comprises a seed region, in which any 3 or more non-consecutive nucleotides in the seed region are independently 2′-deoxy. In some embodiments, a provided single-stranded RNAi agent comprises a seed region, in which any 4 or more non-consecutive nucleotides in the seed region are independently 2′-deoxy.

In some embodiments, a provided single-stranded RNAi agent comprises a seed region, in which any 3 of the nucleotides in the seed region are independently 2′-deoxy.

In some embodiments, a provided single-stranded RNAi agent comprises a seed region, in which any one or more internucleotidic linkages in the seed region are independently PO (phosphodiester). In some embodiments, a provided single-stranded RNAi agent comprises a seed region, in which any 2 or more internucleotidic linkages in the seed region are independently PO. In some embodiments, a provided single-stranded RNAi agent comprises a seed region, in which any 3 or more internucleotidic linkages in the seed region are independently PO. In some embodiments, a provided single-stranded RNAi agent comprises a seed region, in which any 4 or more internucleotidic linkages in the seed region are independently PO. In some embodiments, a provided single-stranded RNAi agent comprises a seed region, in which any 5 or more internucleotidic linkages in the seed region are independently PO. In some embodiments, a provided single-stranded RNAi agent comprises a seed region, in which any 6 or more internucleotidic linkages in the seed region are independently PO. In some embodiments, a provided single-stranded RNAi agent comprises a seed region, in which 7 of N2 to N7 are independently PO.

In some embodiments, a provided single-stranded RNAi agent comprises a seed region, in which any one or more internucleotidic linkages in the seed region are independently PS (phosphorothioate). In some embodiments, a provided single-stranded RNAi agent comprises a seed region, in which any 2 or more internucleotidic linkages in the seed region are independently PS. In some embodiments, a provided single-stranded RNAi agent comprises a seed region, in which any 3 or more internucleotidic linkages in the seed region are independently PS. In some embodiments, a provided single-stranded RNAi agent comprises a seed region, in which any 4 or more internucleotidic linkages in the seed region are independently PS. In some embodiments, a provided single-stranded RNAi agent comprises a seed region, in which any 5 or more internucleotidic linkages in the seed region are independently PS. In some embodiments, a provided single-stranded RNAi agent comprises a seed region, in which any 6 or more internucleotidic linkages in the seed region are independently PS. In some embodiments, a provided single-stranded RNAi agent comprises a seed region, in which 7 of N2 to N7 are independently PS.

In some embodiments, a provided single-stranded RNAi agent comprises a seed region, in which any one or more internucleotidic linkages in the seed region are independently Sp (phosphorothioate in the Sp configuration). In some embodiments, a provided single-stranded RNAi agent comprises a seed region, in which any 2 or more internucleotidic linkages in the seed region are independently Sp. In some embodiments, a provided single-stranded RNAi agent comprises a seed region, in which any 3 or more internucleotidic linkages in the seed region are independently Sp. In some embodiments, a provided single-stranded RNAi agent comprises a seed region, in which any 4 or more internucleotidic linkages in the seed region are independently Sp. In some embodiments, a provided single-stranded RNAi agent comprises a seed region, in which any 5 or more internucleotidic linkages in the seed region are independently Sp. In some embodiments, a provided single-stranded RNAi agent comprises a seed region, in which any 6 or more internucleotidic linkages in the seed region are independently Sp. In some embodiments, a provided single-stranded RNAi agent comprises a seed region, in which 7 of N2 to N7 are independently Sp.

In some embodiments, a provided single-stranded RNAi agent comprises a seed region, in which any one or more internucleotidic linkages in the seed region are independently Rp (phosphorothioate in the Rp configuration). In some embodiments, a provided single-stranded RNAi agent comprises a seed region, in which any 2 or more internucleotidic linkages in the seed region are independently Rp. In some embodiments, a provided single-stranded RNAi agent comprises a seed region, in which any 3 or more internucleotidic linkages in the seed region are independently Rp. In some embodiments, a provided single-stranded RNAi agent comprises a seed region, in which any 4 or more internucleotidic linkages in the seed region are independently Rp. In some embodiments, a provided single-stranded RNAi agent comprises a seed region, in which any 5 or more internucleotidic linkages in the seed region are independently Rp. In some embodiments, a provided single-stranded RNAi agent comprises a seed region, in which any 6 or more internucleotidic linkages in the seed region are independently Rp. In some embodiments, a provided single-stranded RNAi agent comprises a seed region, in which 7 of N2 to N7 are independently Rp.

In some embodiments, a provided single-stranded RNAi agent comprises a seed region, in which any one or more non-consecutive internucleotidic linkages in the seed region are independently PO. In some embodiments, a provided single-stranded RNAi agent comprises a seed region, in which any 2 or more non-consecutive internucleotidic linkages in the seed region are independently PO. In some embodiments, a provided single-stranded RNAi agent comprises a seed region, in which any 3 or more non-consecutive internucleotidic linkages in the seed region are independently PO. In some embodiments, a provided single-stranded RNAi agent comprises a seed region, in which any 4 or more non-consecutive internucleotidic linkages in the seed region are independently PO.

In some embodiments, a provided single-stranded RNAi agent comprises a seed region, in which any one or more non-consecutive internucleotidic linkages in the seed region are independently Sp. In some embodiments, a provided single-stranded RNAi agent comprises a seed region, in which any 2 or more non-consecutive internucleotidic linkages in the seed region are independently Sp. In some embodiments, a provided single-stranded RNAi agent comprises a seed region, in which any 3 or more non-consecutive internucleotidic linkages in the seed region are independently Sp. In some embodiments, a provided single-stranded RNAi agent comprises a seed region, in which any 4 or more non-consecutive internucleotidic linkages in the seed region are independently Sp.

In some embodiments, a provided single-stranded RNAi agent comprises a seed region, in which any one or more non-consecutive internucleotidic linkages in the seed region are independently Rp. In some embodiments, a provided single-stranded RNAi agent comprises a seed region, in which any 2 or more non-consecutive internucleotidic linkages in the seed region are independently Rp. In some embodiments, a provided single-stranded RNAi agent comprises a seed region, in which any 3 or more non-consecutive internucleotidic linkages in the seed region are independently Rp. In some embodiments, a provided single-stranded RNAi agent comprises a seed region, in which any 4 or more non-consecutive internucleotidic linkages in the seed region are independently Rp.

In some embodiments, a provided single-stranded RNAi agent comprises a seed region, in which any one or more non-consecutive internucleotidic linkages in the seed region are independently PS. In some embodiments, a provided single-stranded RNAi agent comprises a seed region, in which any 2 or more non-consecutive internucleotidic linkages in the seed region are independently PS. In some embodiments, a provided single-stranded RNAi agent comprises a seed region, in which any 3 or more non-consecutive internucleotidic linkages in the seed region are independently PS. In some embodiments, a provided single-stranded RNAi agent comprises a seed region, in which any 4 or more non-consecutive internucleotidic linkages in the seed region are independently PS.

In some embodiments, a provided single-stranded RNAi agent comprises a seed region, in which the pattern of 2′ modifications of the nucleotides comprises the pattern of 2′ modifications of the nucleotides in the seed region of any single-stranded RNAi format shown in FIG. 1.

In some embodiments, a provided single-stranded RNAi agent comprises a seed region, in which the pattern of 2′ modifications of the nucleotides comprises: fmfmfm, where f is 2′-F and m is 2′-OMe.

In some embodiments, a provided single-stranded RNAi agent comprises a seed region, in which the pattern of 2′ modifications of the nucleotides comprises: fmfmfmf, where f is 2′-F and m is 2′-OMe.

In some embodiments, a provided single-stranded RNAi agent comprises a seed region, in which the pattern of 2′ modifications of the nucleotides comprises: mfmfmf, where f is 2′-F and m is 2′-OMe.

In some embodiments, a provided single-stranded RNAi agent comprises a seed region, in which the pattern of 2′ modifications of the nucleotides comprises: fmfmf, where f is 2′-F and m is 2′-OMe.

In some embodiments, a provided single-stranded RNAi agent comprises a seed region, in which the pattern of 2′ modifications of the nucleotides comprises: mfmfmf, where f is 2′-F and m is 2′-OMe.

In some embodiments, a provided single-stranded RNAi agent comprises a seed region, in which the pattern of 2′ modifications of the nucleotides comprises: mfmfm, where f is 2′-F and m is 2′-OMe.

In some embodiments, a provided single-stranded RNAi agent comprises a seed region, in which the pattern of 2′ modifications of the nucleotides comprises: mfmfmfm, where f is 2′-F and m is 2′-OMe.

In some embodiments, a provided single-stranded RNAi agent comprises a seed region, in which the pattern of 2′ modifications of the nucleotides comprises: fmfmf, where f is 2′-F and m is 2′-OMe.

In some embodiments, a provided single-stranded RNAi agent comprises a seed region, in which the pattern of 2′ modifications of the nucleotides comprises: mfmfmfm, where f is 2′-F and m is 2′-OMe.

In some embodiments, a provided single-stranded RNAi agent comprises a seed region, in which the pattern of 2′ modifications of the nucleotides comprises: mfMfmf, where f is 2′-F, m is 2′-OMe, and M is 2′-MOE.

In some embodiments, a provided single-stranded RNAi agent comprises a seed region, in which the pattern of 2′ modifications of the nucleotides comprises: mfMfmfm, where f is 2′-F, m is 2′-OMe, and M is 2′-MOE.

In some embodiments, a provided single-stranded RNAi agent comprises a seed region, in which the pattern of 2′ modifications of the nucleotides comprises: mfMfm, where f is 2′-F, m is 2′-OMe, and M is 2′-MOE.

In some embodiments, a provided single-stranded RNAi agent comprises a seed region, in which the pattern of 2′ modifications of the nucleotides comprises: mfMf, where f is 2′-F, m is 2′-OMe, and M is 2′-MOE.

In some embodiments, a provided single-stranded RNAi agent comprises a seed region, in which the pattern of 2′ modifications of the nucleotides comprises: mfmfMf, where f is 2′-F, m is 2′-OMe, and M is 2′-MOE.

In some embodiments, a provided single-stranded RNAi agent comprises a seed region, in which the pattern of 2′ modifications of the nucleotides comprises: mfmfMfm, where f is 2′-F, m is 2′-OMe, and M is 2′-MOE.

In some embodiments, a provided single-stranded RNAi agent comprises a seed region, in which the pattern of 2′ modifications of the nucleotides comprises: mfmfM, where f is 2′-F, m is 2′-OMe, and M is 2′-MOE.

In some embodiments, a provided single-stranded RNAi agent comprises a seed region, in which the pattern of 2′ modifications of the nucleotides comprises: mfMfMf, where f is 2′-F, m is 2′-OMe, and M is 2′-MOE.

In some embodiments, a provided single-stranded RNAi agent comprises a seed region, in which the pattern of 2′ modifications of the nucleotides comprises: mfMfMfM, where f is 2′-F, m is 2′-OMe, and M is 2′-MOE.

In some embodiments, a provided single-stranded RNAi agent comprises a seed region, in which the pattern of 2′ modifications of the nucleotides comprises: mfMfM, where f is 2′-F, m is 2′-OMe, and M is 2′-MOE.

In some embodiments, a provided single-stranded RNAi agent comprises a seed region, in which the pattern of 2′ modifications of the nucleotides comprises: fdfdfd, where d is 2′-deoxy and m is 2′-OMe.

In some embodiments, a provided single-stranded RNAi agent comprises a seed region, in which the pattern of 2′ modifications of the nucleotides comprises: fdfdfdf, where d is 2′-deoxy and m is 2′-OMe.

In some embodiments, a provided single-stranded RNAi agent comprises a seed region, in which the pattern of 2′ modifications of the nucleotides comprises: dfdfdf, where d is 2′-deoxy and m is 2′-OMe.

In some embodiments, a provided single-stranded RNAi agent comprises a seed region, in which the pattern of 2′ modifications of the nucleotides comprises: fdfdf, where d is 2′-deoxy and m is 2′-OMe.

In some embodiments, a provided single-stranded RNAi agent comprises a seed region, in which the pattern of 2′ modifications of the nucleotides comprises: ffmmmm, where f is 2′-F and m is 2′-OMe.

In some embodiments, a provided single-stranded RNAi agent comprises a seed region, in which the pattern of 2′ modifications of the nucleotides comprises: fmmmm, where f is 2′-F and m is 2′-OMe.

In some embodiments, a provided single-stranded RNAi agent comprises a seed region, in which the pattern of 2′ modifications of the nucleotides comprises: ffmmm, where f is 2′-F and m is 2′-OMe.

In some embodiments, a provided single-stranded RNAi agent comprises a seed region, in which the pattern of 2′ modifications of the nucleotides comprises: ffmm, where f is 2′-F and m is 2′-OMe.

In some embodiments, a provided single-stranded RNAi agent comprises a seed region, in which the pattern of 2′ modifications of the nucleotides comprises: ffmmmmmm, where f is 2′-F and m is 2′-OMe.

In some embodiments, a provided single-stranded RNAi agent comprises a seed region, in which the pattern of 2′ modifications of the nucleotides comprises: ffmmmmm, where f is 2′-F and m is 2′-OMe.

In some embodiments, a provided single-stranded RNAi agent comprises a seed region, in which the pattern of 2′ modifications of the nucleotides comprises: mmmm, where m is 2′-OMe.

In some embodiments, a provided single-stranded RNAi agent comprises a seed region, in which the pattern of 2′ modifications of the nucleotides comprises: mmmmm, where m is 2′-OMe.

In some embodiments, a provided single-stranded RNAi agent comprises a seed region, in which the pattern of 2′ modifications of the nucleotides comprises: mmmmmm, where m is 2′-OMe.

In some embodiments, a provided single-stranded RNAi agent comprises a seed region, in which the pattern of 2′ modifications of the nucleotides comprises: mfmfm, where f is 2′-F and m is 2′-OMe.

In some embodiments, a provided single-stranded RNAi agent comprises a seed region, in which the pattern of internucleotidic linkages comprises the pattern of internucleotidic linkages in the seed region of any single-stranded RNAi format shown in FIG. 1.

In some embodiments, a provided single-stranded RNAi agent comprises a seed region, in which the pattern of internucleotidic linkages comprises: XOXOXO, where X is phosphorothioate and O is phosphodiester.

In some embodiments, a provided single-stranded RNAi agent comprises a seed region, in which the pattern of internucleotidic linkages comprises: XOXOXOX, where X is phosphorothioate and O is phosphodiester.

In some embodiments, a provided single-stranded RNAi agent comprises a seed region, in which the pattern of internucleotidic linkages comprises: OXOXOX, where X is phosphorothioate and O is phosphodiester.

In some embodiments, a provided single-stranded RNAi agent comprises a seed region, in which the pattern of internucleotidic linkages comprises: XOXOX, where X is phosphorothioate and O is phosphodiester.

In some embodiments, a provided single-stranded RNAi agent comprises a seed region, in which the pattern of internucleotidic linkages comprises: OXOXOX, where X is phosphorothioate and O is phosphodiester.

In some embodiments, a provided single-stranded RNAi agent comprises a seed region, in which the pattern of internucleotidic linkages comprises: OXOXOXO, where X is phosphorothioate and O is phosphodiester.

In some embodiments, a provided single-stranded RNAi agent comprises a seed region, in which the pattern of internucleotidic linkages comprises: OXOXO, where X is phosphorothioate and O is phosphodiester.

In some embodiments, a provided single-stranded RNAi agent comprises a seed region, in which the pattern of internucleotidic linkages comprises: OXOX, where X is phosphorothioate and O is phosphodiester.

In some embodiments, a provided single-stranded RNAi agent comprises a seed region, in which the pattern of internucleotidic linkages comprises: XXOXOX, where X is phosphorothioate and O is phosphodiester.

In some embodiments, a provided single-stranded RNAi agent comprises a seed region, in which the pattern of internucleotidic linkages comprises: XXOXOXO, where X is phosphorothioate and O is phosphodiester.

In some embodiments, a provided single-stranded RNAi agent comprises a seed region, in which the pattern of internucleotidic linkages comprises: XXOXO, where X is phosphorothioate and O is phosphodiester.

In some embodiments, a provided single-stranded RNAi agent comprises a seed region, in which the pattern of internucleotidic linkages comprises: XXOX, where X is phosphorothioate and O is phosphodiester.

In some embodiments, a provided single-stranded RNAi agent comprises a seed region, in which the pattern of internucleotidic linkages comprises: OOOOOO, where O is phosphodiester.

In some embodiments, a provided single-stranded RNAi agent comprises a seed region, in which the pattern of internucleotidic linkages comprises: OOOOO, where O is phosphodiester.

In some embodiments, a provided single-stranded RNAi agent comprises a seed region, in which the pattern of internucleotidic linkages comprises: OOOO, where O is phosphodiester.

In some embodiments, a provided single-stranded RNAi agent comprises a seed region, in which the pattern of internucleotidic linkages comprises: SOSOSO, where S is a phosphorothioate in the Sp configuration and O is phosphodiester.

In some embodiments, a provided single-stranded RNAi agent comprises a seed region, in which the pattern of internucleotidic linkages comprises: SOSOSOS, where S is a phosphorothioate in the Sp configuration and O is phosphodiester.

In some embodiments, a provided single-stranded RNAi agent comprises a seed region, in which the pattern of internucleotidic linkages comprises: OSOSOS, where S is a phosphorothioate in the Sp configuration and O is phosphodiester.

In some embodiments, a provided single-stranded RNAi agent comprises a seed region, wherein the seed region comprises a phosphorothioate in the Sp configuration.

In some embodiments, a provided single-stranded RNAi agent comprises a seed region, wherein the seed region comprises a phosphorothioate in the Sp configuration and a phosphodiester.

In some embodiments, a provided single-stranded RNAi agent comprises a seed region, in which the pattern of internucleotidic linkages comprises: SOSOS, where S is a phosphorothioate in the Sp configuration and O is phosphodiester.

In some embodiments, a provided single-stranded RNAi agent comprises a seed region, in which the pattern of internucleotidic linkages comprises: SOSSSS, where S is a phosphorothioate in the Sp configuration and O is phosphodiester.

In some embodiments, a provided single-stranded RNAi agent comprises a seed region, in which the pattern of internucleotidic linkages comprises: SOSSS, where S is a phosphorothioate in the Sp configuration and O is phosphodiester.

In some embodiments, a provided single-stranded RNAi agent comprises a seed region, in which the pattern of internucleotidic linkages comprises: SOSS, where S is a phosphorothioate in the Sp configuration and O is phosphodiester.

In some embodiments, a provided single-stranded RNAi agent comprises a seed region, in which the pattern of internucleotidic linkages comprises the pattern of internucleotidic linkages in the seed region of a first single-stranded RNAi format shown in FIG. 1; and the pattern of 2′ modifications of the nucleotides comprises the pattern of 2′ modifications of the nucleotides in the seed region of a second single-stranded RNAi format shown in FIG. 1.

In some embodiments, a provided single-stranded RNAi agent comprises a seed region, in which the pattern of internucleotidic linkages comprises the pattern of internucleotidic linkages in the seed region of a first single-stranded RNAi format shown in FIG. 1; and the pattern of 2′ modifications of the nucleotides comprises the pattern of 2′ modifications of the nucleotides in the seed region of the first single-stranded RNAi format shown in FIG. 1.

Post-Seed Region of an APOC3 Oligonucleotide, Including a Single-Stranded RNAi Agent

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any post-seed region or portion or structural element thereof described herein or known in the art.

In some embodiments of a single-stranded RNAi agent, a post-seed region comprises: a sequence of nucleotides comprising at least 1 2′-F modifications. In some embodiments of a single-stranded RNAi agent, a post-seed region comprises: a sequence of nucleotides comprising at least 2 to 20 2′-F modifications.

In some embodiments of a single-stranded RNAi agent, a post-seed region comprises: a sequence of nucleotides comprising at least 1 2′-OMe modifications. In some embodiments of a single-stranded RNAi agent, a post-seed region comprises: a sequence of nucleotides comprising at least 2 to 20 2′-OMe modifications.

In some embodiments of a single-stranded RNAi agent, a post-seed region comprises: a sequence of nucleotides comprising at least 1 total 2′-OMe and/or 2′-F modifications. In some embodiments of a single-stranded RNAi agent, a post-seed region comprises: a sequence of nucleotides comprising at least 2 to 20 total 2′-OMe and/or 2′-F modifications.

In some embodiments of a single-stranded RNAi agent, a post-seed region comprises: at least 2 to 10 consecutive pairs of nucleotides having 2′-F and 2′-OMe or 2′-OMe and 2′-F modifications.

In some embodiments of a single-stranded RNAi agent, a post-seed region comprises a pattern of alternating 2′-modifications, wherein the pattern comprises 2′-F, 2′-OMe, 2′-F, 2′-OMe. In some embodiments of a single-stranded RNAi agent, a post-seed region comprises a pattern of alternating 2′-modifications, wherein the pattern comprises 2′-F, 2′-OMe, 2′-F, 2′-OMe, 2′-F, 2′-OMe. In some embodiments of a single-stranded RNAi agent, a post-seed region comprises a pattern of alternating 2′-modifications, wherein the pattern comprises 2′-F, 2′-OMe, 2′-F, 2′-OMe, 2′-F, 2′-OMe, 2′-F, 2′-OMe. In some embodiments of a single-stranded RNAi agent, a post-seed region comprises a pattern of alternating 2′-modifications, wherein the pattern comprises 2′-F, 2′-OMe, 2′-F, 2′-OMe, 2′-F, 2′-OMe, 2′-F, 2′-OMe, 2′-F, 2′-OMe. In some embodiments of a single-stranded RNAi agent, a post-seed region comprises a pattern of alternating 2′-modifications, wherein the pattern comprises 2′-F, 2′-OMe, 2′-F, 2′-OMe, 2′-F, 2′-OMe, 2′-F, 2′-OMe, 2′-F, 2′-OMe, 2′-F, 2′-OMe. In some embodiments of a single-stranded RNAi agent, a post-seed region comprises a pattern of alternating 2′-modifications, wherein the pattern comprises 2′-F, 2′-OMe, 2′-F, 2′-OMe, 2′-F, 2′-OMe, 2′-F, 2′-OMe, 2′-F, 2′-OMe, 2′-F, 2′-OMe, 2′-F, 2′-OMe. In some embodiments of a single-stranded RNAi agent, a post-seed region comprises a pattern of alternating 2′-modifications, wherein the pattern comprises 2′-F, 2′-OMe, 2′-F, 2′-OMe, 2′-F, 2′-OMe, 2′-F, 2′-OMe, 2′-F, 2′-OMe, 2′-F, 2′-OMe, 2′-F, 2′-OMe, 2′-F, 2′-OMe. In some embodiments of a single-stranded RNAi agent, a post-seed region comprises a pattern of alternating 2′-modifications, wherein the pattern comprises 2′-F, 2′-OMe, 2′-F, 2′-OMe, 2′-F, 2′-OMe, 2′-F, 2′-OMe, 2′-F, 2′-OMe, 2′-F, 2′-OMe, 2′-F, 2′-OMe, 2′-F, 2′-OMe, 2′-F, 2′-OMe. In some embodiments of a single-stranded RNAi agent, a post-seed region comprises a pattern of alternating 2′-modifications, wherein the pattern comprises fmfmfmfmfm.

In some embodiments of a single-stranded RNAi agent, a post-seed region comprises a pattern of alternating 2′-modifications, wherein the pattern comprises mfmf, mfmfmf, mfmfmfmf, mfmfmfmfmf, mfmfmfmfmf, mfmfmfmfmfmf, mfmfmfmfmfmfmf, mfmfmfmfmfmfmfmf, mfmfmfmfmfmfmfmfmf, where m is 2′-OMe and f is 2′-F.

In some embodiments, each nucleotide in the seed region is 2′-OMe.

In some embodiments, each nucleotide in the seed region is 2′-OMe, and each nucleotide in the post-seed region is 2′-OMe.

In some embodiments, one nucleotide in the seed region is 2′-F and each other nucleotide in the seed region is 2′-OMe.

In some embodiments, one nucleotide in the seed region is 2′-F and each other nucleotide in the seed region is 2′-OMe, and one nucleotide in the post-seed region is 2′-F and each other nucleotide in the post-seed region is 2′-OMe.

In some embodiments, the nucleotide at position 2 (counting from the 5′-end) is 2′-F and each other nucleotide in the seed region is 2′-OMe, and one nucleotide in the post-seed region is 2′-F and each other nucleotide in the post-seed region is 2′-OMe.

In some embodiments, the nucleotide at position 2 (counting from the 5′-end) is 2′-F and each other nucleotide in the seed region is 2′-OMe, and the nucleotide at position 14 (counting from the 5′-end) is 2′-F and each other nucleotide in the post-seed region is 2′-OMe.

Without wishing to be bound by any particular theory, the present disclosure suggests that, in at least some cases, reducing the number of 2′-F nucleotides (e.g., replacing them with 2′-OMe, 2′-deoxy or any other nucleotide which is not 2′-F) can allow in vitro potency, and allow or increase stability, while reducing potential toxicity related to 2′-F.

In some embodiments, a post-seed region comprises at least 1, 2, 3, 4, 5 6, 7, 8 or 9 phosphorothioates and/or at least 1, 2, 3, 4, 5 6, 7, 8 or 9 phosphodiester internucleotidic linkages.

In some embodiments of a single-stranded RNAi agent, a post-seed region comprises one or more sets of consecutive phosphorothioates and/or one or more sets of consecutive phosphodiesters.

In some embodiments, a single-stranded RNAi agent comprises a post-seed region which comprises a span of sugars having a pattern of modifications of any of: mfmfmfmfmfmfm, mfmfmfmfmfm, mfmfmfmfm, mfmfmfm, mfmfm, mfm, fmfmfmfmfmfm, fmfmfmfmfm, fmfmfmfm, fmfmfm, and fmfm, wherein m is 2′-OMe and f is 2′-F.

In some embodiments, a single-stranded RNAi agent comprises a post-seed region which comprises a span of sugars having a pattern of modifications of any of: dddfdfdfdfdfd, dddfdfdfdfd, dddfdfdfd, dddfdfd, and dddfd, wherein d is 2′-deoxy and f is 2′-F.

In some embodiments, a single-stranded RNAi agent comprises a post-seed region which comprises a span of sugars having a pattern of modifications of any of: dfdfdfdfdfdfd, fdfdfdfdfdfd, and fdfdfdfdfd.

In some embodiments, a single-stranded RNAi agent comprises a post-seed region which comprises a span of sugars having a pattern of modifications of any of: fdfdfdfd, fdfdfd, and fdfd.

In some embodiments, a single-stranded RNAi agent comprises a post-seed region which comprises a span of internucleotidic linkages having a pattern of any of: XOXOXOXOOOO, OXOXOXOOOO, XOXOXOXOOO, XOXOXOXOO, XOXOXOOOO, OXOXOXOO, XOXOXOOO, OXOXOOOO, OXOXOOO, and XOXOOO, wherein O is phosphodiester and X is a stereorandom phosphorothioate.

In some embodiments, a single-stranded RNAi agent comprises a post-seed region which comprises a span of internucleotidic linkages having a pattern of OOOOOOO.

In some embodiments, a single-stranded RNAi agent comprises a post-seed region which comprises a span of internucleotidic linkages having a pattern of any of: OOOOOO, OOOOO, OOOO, and OOO.

In some embodiments, a single-stranded RNAi agent comprises a post-seed region which comprises a span of internucleotidic linkages having a pattern of any of: OOOOOOOOXXXXXX, OOOOOOOOXXXXX, OOOOOOOOXXXX, OOOOOOOOXXX, OOOOOOOOXX, OOOOOOOOX, OOOOOOOXXXXXX, OOOOOOXXXXXX, OOOOOXXXXXX, OOOOXXXXXX, OOOXXXXXX, OOXXXXXX, OXXXXXX, OOOOOOX, OOOOOX, OOOOX, and OOOX.

In some embodiments, a single-stranded RNAi agent comprises a post-seed region which comprises a span of internucleotidic linkages having a pattern of any of: OOOOOOOXXXXX, OOOOOOOXXXX, OOOOOOOXXX, OOOOOOXXXXX, OOOOOOXXXX, OOOOOXXXXX, OOOOOXXXX, OOOOOXXX, OOOOXXXX, OOOOXXX, OOOXXXXX, OOOXXXX, and OOOXXX.

In some embodiments, a single-stranded RNAi agent comprises a post-seed region which comprises at least one chiral internucleotidic linkage. In some embodiments, a single-stranded RNAi agent comprises a post-seed region which comprises at least one chirally controlled internucleotidic linkage. In some embodiments, a single-stranded RNAi agent comprises a post-seed region which comprises at least one chirally controlled internucleotidic linkage which is a phosphorothioate. In some embodiments, a single-stranded RNAi agent comprises a post-seed region which comprises at least one chirally controlled internucleotidic linkage which is a phosphorothioate in the Sp configuration. In some embodiments, a single-stranded RNAi agent comprises a post-seed region which comprises at least one chirally controlled internucleotidic linkage which is a phosphorothioate in the Rp configuration.

In some embodiments, a single-stranded RNAi agent comprises a post-seed region which comprises a span of internucleotidic linkages having a pattern of any of: OSSSOSSSSSSSSSS, OSSSOSSSSSSSSS, OSSSOSSSSSSSS, OSSSOSSSSSSS, OSSSOSSSSSS, OSSSOSSSSS, OSSSOSSSS, OSSSOSSS, OSSSOSS, OSSSOS, and OSSSO, wherein O is phosphodiester and S is a phosphorothioate in the Sp configuration.

In some embodiments, a single-stranded RNAi agent comprises a post-seed region which comprises a span of internucleotidic linkages having a pattern of any of: OSOSOSOSSSSSSSS, OSOSOSOSSSSSSS, OSOSOSOSSSSSS, OSOSOSOSSSSS, OSOSOSOSSSS, OSOSOSOSSS, and OSOSOSOSS.

In some embodiments, a single-stranded RNAi agent comprises a post-seed region which comprises a span of internucleotidic linkages having a pattern of any of: SOSOSOSSSSSSSS, OSOSOSSSSSSSS, SOSOSSSSSSSS, OSOSSSSSSSS, SOSSSSSSSS, and OSSSSSSSS.

In some embodiments, a single-stranded RNAi agent comprises a post-seed region which comprises a span of internucleotidic linkages having a pattern of any of: SOSOSOSSSSSSSS, SOSOSOSSSSSSS, SOSOSOSSSSSS, SOSOSOSSSSS, SOSOSOSSSS, SOSOSOSSS, SSSSSSSS, SSSSSSS, SSSSSS, SSSSS, SSSS, SSS, and SS.

In some embodiments, a single-stranded RNAi agent comprises a post-seed region which comprises a span of internucleotidic linkages having a pattern of any of: OSSSSSO, OSSSSS, OSSSS, SSSSSO, SSSSO, and SSSO.

In some embodiments, a single-stranded RNAi agent comprises a post-seed region which comprises a span of internucleotidic linkages having a pattern of any of: SOSOSOSOSOOOOOOS, SOSOSOSOSOOOOOO, SOSOSOSOSOOOOO, SOSOSOSOSOOOO, SOSOSOSOSOOO, and SOSOSOSOSOO.

In some embodiments, a single-stranded RNAi agent comprises a post-seed region which comprises a span of internucleotidic linkages having a pattern of any of: OSOSOSOSOOOOOOS, SOSOSOSOOOOOOS, OSOSOSOOOOOOS, SOSOSOOOOOOS, OSOSOOOOOOS, SOSOOOOOOS, and OSOOOOOOS.

In some embodiments, a single-stranded RNAi agent comprises a post-seed region which comprises a span of internucleotidic linkages having a pattern of any of: XOOOXOOXXXXX, XOOOXOOXXXX, XOOOXOOXXX, XOOOXOOXX, XOOOXOOX, XOOOXOO, OOOXOOXXXXX, OOXOOXXXXX, OXOOXXXXX, XOOXXXXX, and OOXXXXX.

In some embodiments, a single-stranded RNAi agent comprises a post-seed region which comprises a span of internucleotidic linkages having a pattern of any of: XOXOXOXXXXXX, XOXOXOXXXXX, XOXOXOXXXX, XOXOXOXXX, XOXOXOXX, XOXOXOX, OXOXOXXXXXX, XOXOXXXXXX, OXOXXXXXX, XOXXXXXX, and OXXXXXX.

In some embodiments, a single-stranded RNAi agent comprises a post-seed region which comprises a span of internucleotidic linkages having a pattern of any of: XXXOOOXOXOXXX, XXXOOOXOXOXX, XXXOOOXOXOX, XXXOOOXOXO, XXXOOOXOX, XXXOOOXO, XXOOOXOXOXXX, XOOOXOXOXXX, OOOXOXOXXX, OOXOXOXXX, OXOXOXXX, XOXOXXX, XXOOOXOXOXX, XXOOOXOXOX, XOOOXOXOXX, and XOOOXOXOX.

In some embodiments, a single-stranded RNAi agent comprises a post-seed region which comprises a span of internucleotidic linkages having a pattern of any of: XOOOXOXO, XOOOXOX, XOOOXO, OOOXOXO, OOOXOX, and OOOXO.

In some embodiments, a single-stranded RNAi agent comprises a post-seed region which comprises a span of internucleotidic linkages having a pattern of any of: XOXOOOXOXOXXX, XOXOOOXOXOXX, XOXOOOXOXOX, XOXOOOXOXO, XOXOOOXOX, XOXOOOXO, XOXOOOX, OXOOOXOXOXXX, XOOOXOXOXXX, OOOXOXOXXX, OOXOXOXXX, OXOXOXXX, OXOOOXOXOXX, OXOOOXOXOX, XOOOXOXOXX, and XOOOXOXOX.

In some embodiments, a single-stranded RNAi agent comprises a post-seed region which comprises a span of internucleotidic linkages having a pattern of any of: OOOOOOS, OOOOOSO, OOOOSOO, OOOSOOO, OOSOOOO, OSOOOOO, and SOOOOOO.

In some embodiments, a hybrid oligonucleotide comprises a (a) seed region capable of annealing to a first complementary target mRNA region; and (b) a post-seed region comprising a 2′-deoxy region, wherein the hybrid oligonucleotide is capable of directing both RNA interference and RNase H-mediated knockdown, wherein the 2′-deoxy region comprises at least 5 consecutive 2′-deoxy. In some embodiments, the 2′-deoxy can be DNA, or a modified nucleotide, e.g., a modified nucleotide with a 2′-deoxy, wherein the DNA or modified nucleotide comprise a natural sugar and/or a natural base, and/or a modified base, and/or any internucleotidic linkage. In some embodiments, the 2′-deoxy region comprises a stretch of consecutive nucleotides, wherein each nucleotide is 2′-deoxy and each internucleotidic linkage is a phosphorothioate. In some embodiments, the 2′-deoxy region comprises a stretch of consecutive nucleotides of at least 5, 6, 7, 8, 9, 10, 11, 12, 13, 14, 15, 16, 17, 18, 19, 20 or 21 nucleotides, wherein each nucleotide is 2′-deoxy and each internucleotidic linkage is a phosphorothioate.

As non-limiting examples of a post-seed region in a single-stranded RNAi agent: Formats 2, 7, 8, 9, 12 and 13 (which each comprise a set of 6 consecutive phosphodiesters; and a set of 6 consecutive phosphodithioates), Format 3 (6 consecutive phosphorodithioates), Formats 4, 5 and 6, Formats 10 and 11; and various other single-stranded RNAi agents disclosed herein.

In some embodiments of a single-stranded RNAi agent, a post-seed region comprises a mismatch at the most 3′ position.

In some embodiments, a provided single-stranded RNAi agent can comprise a mismatch at any one or more of: the 5′ position, either or both of the 3′-terminal dinucleotide, and the most 3′ position of the region between the seed region and the 3′-end region.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any post-seed region or portion or structural element thereof described herein or known in the art.

3′-End Region of an APOC3 Oligonucleotide, Including a Single-Stranded RNAi Agent

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides can comprise any 3′-end region described herein or known in the art.

In some embodiments, the 3′-end region of an APOC3 oligonucleotide is such that the oligonucleotide is capable of directing a decrease in the expression and/or level of a target gene or its gene product.

In some embodiments, the 3′-end region of a RNAi agent is such that the RNAi agent is capable of directing RNA interference of a specific target transcript in a sequence-specific manner.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any 3′-end region and/or 3′-terminal dinucleotide and/or 3′-end cap described herein or known in the art. In some embodiments, a 3′-end region can comprise a GalNAc moiety. In some embodiments, a GalNAc moiety is any GalNAc, or variant, derivative or modification thereof, as described herein or known in the art.

In some embodiments, a 3′-end region and/or 3′-terminal dinucleotide and/or 3′-end cap performs two functions: (a) decreasing the sensitivity of the oligonucleotide to exo- and/or endonucleases; and (b) allowing the function of the oligonucleotide, wherein the function is directing RNA interference, directing RNase H-mediated knockdown, or directing both RNA interference and RNase H-mediated knockdown.

Thus, the 3′-end region of the single-stranded RNAi agent can comprise a 3′-terminal dinucleotide and/or a 3′-end cap.

In a mammalian cell, Dicer reportedly processes double-stranded RNA (dsRNA) into 19-23 base pair siRNAs, which comprise a double-stranded region, with each strand terminating in a single-stranded 3′ overhang, which can be 1 to 4 nt long, but is typically a 3′-terminal dinucleotide. Bernstein et al. 2001 Nature 409: 363; Elbashir et al. 2001 Nature 411: 494-498; Elbashir et al. 2001 EMBO J. 20: 6877. The two dinucleotide overhangs reportedly do not contribute to target specificity. They do, however, reportedly help protect the ends of the siRNA from nuclease degradation and sometimes improve activity. Elbashir et al. 2001 Nature 411: 494-498; Elbashir et al. 2001 EMBO J. 20: 6877-6888; and Kraynack et al. 2006 RNA 12:163-176. Thus, it is reportedly not necessary for a functional double-stranded RNAi agent for a 3′-terminal dinucleotide to comprise a sequence complementary to the target gene sequence.

In artificial double-stranded RNAi agents, the 3′ single-stranded dinucleotide overhangs have reportedly been experimentally replaced with various moieties, including other single-stranded dinucleotides, nucleotides, and 3′-end caps. The 3′-terminal dinucleotides of a 21-mer are reportedly often replaced by an artificial dinucleotide, such as UU, TT, dTdT, sdT, dTsdT, sdTsdT, or sdTdT. Replacing up to four nucleotides on each end of the siRNA with deoxyribonucleotides has reportedly been well tolerated, whereas complete substitution with deoxyribonucleotides results in no RNAi activity. International PCT Publication No. WO 00/44914, and Beach et al. International PCT Publication No. WO 01/68836 preliminarily reported that siRNA may include modifications to either the phosphate-sugar backbone or the nucleoside to include at least one of a nitrogen or sulfur heteroatom. Kreutzer et al. Canadian Patent Application No. 2,359,180, also report certain chemical modifications for use in dsRNA constructs in order to counteract activation of double-stranded RNA-dependent protein kinase PKR, specifically 2′-amino or 2-O-methyl nucleotides, and nucleotides containing a 2′-0 or 4′—C methylene bridge. Additional 3¹-terminal nucleotide overhangs include dT (deoxythimidine), 2′-0,4′—C-ethylene thymidine (eT), and 2-hydroxyethyl phosphate (hp). Other artificial 3′ overhangs (3′-terminal dinucleotides) include dinucleotides of sequences AA, CC, GG, and UG. Elbashir et al. 2001 EMBO J. 20: 6877-6888. In some embodiments, a 3′-terminal dinucleotide reportedly is AA.

Alternatively, in a double-stranded RNAi agent, one or both of the 3′-terminal dinucleotides can reportedly be deleted (and not replaced), leaving a functional siRNA comprising two 19-nt strands forming a 19-bp blunt-ended duplex. Deleting and not replacing the 3′-terminal dinucleotide in a double-stranded RNAi agent reportedly leaves the ends of the strands vulnerable to nucleases; to compensate for this, an artificial 3′-end cap can be added. The 3′-end caps are reportedly non-nucleotidic; they are not nucleotides as they do not comprise all components of a nucleotide (phosphate, sugar and base). The dinucleotide overhangs in a double-stranded RNAi agent can reportedly sometimes functionally be replaced by a 3′-end cap, leaving a blunt-ended 19-bp duplex with one or two 3′-end caps, which can protect the molecule from nucleases. In general, a 3′-end cap reportedly must not prevent RNA interference mediated by the RNAi agent; many 3′-end caps also impart an added advantage, such as increased RNAi activity and/or stability against nucleases.

Without wishing to be bound by any particular theory, the present application notes that in at least some cases, previously-described 3′-end caps reportedly are theorized to interact with a PAZ domain. In some embodiments, a 3′-end cap is reportedly a PAZ ligand. WO 2015/051366. Reportedly, Dicer is an RNase III enzyme and is composed of six recognizable domains. Reportedly, at or near the N-terminus is an approx. 550 aa DExH-box RNA helicase domain, which is immediately followed by a conserved approx. 100 aa domain called DUF283; just C-terminal to DUF283 domain is the PAZ (for Piwi/Argonaute/Zwille) domain, which recognizes single stranded dinucleotide overhangs. Myers et al. 2005. in RNA interference Technology, ed. Appasani, Cambridge University Press, Cambridge UK, p. 29-54; Bernstein et al. 2001 Nature 409: 363-366; and Schauer et al. 2002 Trends Plant Sci. 7: 487-491; Lingel et al. 2003 Nature 426: 465-469; Song et al. 2003 Nature Struct. Biol. 10: 1026-1032; Yan et al. 2003 Nature 426: 468-474; Lingel et al. 2004 Nature Struct. Mol. Biol. 11: 576-577; Ma et al. 2004 Nature 429: 318-322. Reportedly, the PAZ domain in Dicer could also bind RNA to position the catalytic domains for cleavage. Zhang et al. 2004 Cell 1 18: 57-68. In some embodiments, a 3′-end cap is a PAZ ligand which interacts with a PAZ domain.

In some embodiments, a 3′-end cap can allow two functions: (1) allowing RNA interference; and (2) increasing duration of activity and/or biological half-life, which may be accomplished, for example, by increased binding to the PAZ domain of Dicer and/or reducing or preventing degradation of the RNAi agent (e.g., by nucleases such as those in the serum or intestinal fluid).

Various 3′-terminal dinucleotides are described in the oligonucleotides listed in Table 1A.

In some embodiments, a provided single-stranded RNAi agent comprises a pair of 3′-terminal nucleotides. The penultimate nucleotide is 2′-OMe and the 5′ nucleotide is 2′-OMe.

In some embodiments, a provided single-stranded RNAi agent comprises a pair of 3′-terminal nucleotides and the penultimate nucleotide is 2′-deoxy and the 5′ nucleotide is 2′-OMe. Non-limiting examples of single-stranded RNAi agents disclosed herein of this structure include: Formats 10, 11, 13 and 14, FIG. 1; and various other single-stranded RNAi agents disclosed herein.

In some embodiments, a provided single-stranded RNAi agent comprises a pair of 3′-terminal nucleotides and the penultimate nucleotide is 2′-deoxy and the 5′ nucleotide is 2′-OMe, and wherein the penultimate nucleotide comprises a linker.

In some embodiments, a provided single-stranded RNAi agent comprises a pair of 3′-terminal nucleotides and wherein the penultimate nucleotide comprises a linker.

In some embodiments, a provided single-stranded RNAi agent comprises a pair of 3′-terminal nucleotides and the penultimate nucleotide is 2′-deoxy and the 5′ nucleotide is 2′-OMe, and wherein the penultimate nucleotide comprises a linker conjugated to an additional chemical moiety.

In some embodiments, a provided single-stranded RNAi agent comprises a pair of 3′-terminal nucleotides and the penultimate nucleotide is 2′-deoxy T and the 5′ nucleotide is 2′-OMe U, and wherein the penultimate nucleotide comprises a linker conjugated to an additional chemical moiety.

In some embodiments, a provided single-stranded RNAi agent comprises a pair of 3′-terminal nucleotides and wherein the penultimate nucleotide comprises a linker conjugated to an additional chemical moiety selected from: a targeting moiety, a lipid moiety, a carbohydrate moiety, a bicyclic ketal, and a GalNAc moiety.

In some embodiments, a provided single-stranded RNAi agent comprises a pair of 3′-terminal nucleotides and the penultimate nucleotide is 2′-deoxy and the 5′ nucleotide is 2′-OMe, and wherein the penultimate nucleotide comprises a linker conjugated to an additional chemical moiety selected from: a targeting moiety, a lipid moiety, a carbohydrate moiety, a bicyclic ketal, and a GalNAc moiety.

In some embodiments, a provided single-stranded RNAi agent comprises a pair of 3′-terminal nucleotides and the penultimate nucleotide is 2′-deoxy T and the 5′ nucleotide is 2′-OMe U, and wherein the penultimate nucleotide comprises a linker conjugated to an additional chemical moiety selected from: a targeting moiety, a lipid moiety, a carbohydrate moiety, a bicyclic ketal, and a GalNAc moiety.

In some embodiments, a 3′-end region or 3′-end cap comprises a GN3, or any other suitable RNAi agent 3′-end region compound as described in, for example, Allerson et al. 2005 J. Med. Chem. 48: 901-04; Lima et al. 2012 Cell 150: 883-894; Prakash et al. 2015 Nucl. Acids Res. 43: 2993-3011; and/or Prakash et al. 2016 Bioorg. Med. Chem. Lett. 26: 26: 2817-2820.

Various 3′-end caps have been described in the literature.

Generally, a 3′-end cap is joined to the 3′-terminal internucleotidic linkage. The 3′-terminal internucleotidic linkage can be selected from: a phosphodiester, a phosphorothioate, a phosphodithioate, and any internucleotidic linkage described herein.

A 3′-end cap for a provided single-stranded RNAi agent can be selected from, for example, any 3′-end cap described herein.

In some embodiments, a 3′-end cap is selected from: 2′,3′-cyclic phosphate, C3 (or C6, C7, C12) aminolinker, thiol linker, carboxyl linker, non-nucleotidic spacers (C3, C6, C9, C12, abasic, triethylene glycol, hexaethylene glycol), biotin, and fluoresceine.

In some embodiments, a 3′-end cap is selected from any 3′-end cap described in WO 2015/051366, including but not limited to C3, amino C3, C6, C8, C10, and C12. In some embodiments, a 3′-end cap is selected from: Triethylene glycol, Cyclohexyl (or Cyclohex), Phenyl, BP (Biphenyl), Adamantane and Lithocholic acid (or Lithochol), as described in U.S. Pat. Nos. 8,097,716; 8,084,600; 8,344,128; 8,404,831; and 8,404,832.

Various functional 3′-end caps suitable for a provided RNAi agent are described in, for example, EP 1520022 B1; U.S. Pat. Nos. 8,097,716, 8,084,600; 8,404,831; 8,404,832, and 8,344,128; and WO 2015/051366.

In addition, the present disclosure notes that disclosed herein are various 5′-end structures and 3′-end regions, and combinations thereof, which function in single-stranded RNAi agents. However, it is noted, in contrast, many 5′-end structures and 3′-end caps, and combinations thereof, have previously been reported to reduce or eliminate the RNA interference ability of various double-stranded RNAi agents. See, for example, Czauderna et al. 2003 Nucl. Acids Res. 31:2705-2716; Hadwiger et al. 2005, pages 194-206, in RNA interference Technology, ed. K. Appasani, Cambridge University Press, Cambridge, UK; Deleavey et al. 2009 Curr. Prot. Nucl. Acid Chem. 16.3.1-16.3.22; Terrazas et al. 2009 Nucleic Acids Res. 37: 346-353; Harboth et al. 2003 Antisense Nucl. Acid Drug Dev 13: 83-105; Song et al. 2003 Nature Med. 9: 347-351; U.S. Pat. No. 5,998,203; Lipardi et al. 2001 Cell 107: 299-307; Schwarz et al. 2002 Mol. Cell 10: 537-548; and WO 2015/051366.

a bicyclic ketal,

Additional Optional Structural Elements of an APOC3 Oligonucleotide

In some embodiments, an APOC3 oligonucleotide, an APOC3 oligonucleotide capable of directing a decrease in the expression and/or level of a target gene or its gene product can comprise any structural element or pattern thereof described herein or known in the art.

a bicyclic ketal,

Optional Additional Chemical Moiety Conjugated to an APOC3 Oligonucleotide

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides can comprise any optional additional chemical moiety, including but not limited to, a targeting moiety, a lipid moiety, a carbohydrate moiety, a bicyclic ketal, a GalNAc moiety, etc., described herein or known in the art.

In some embodiments, an APOC3 oligonucleotide, an APOC3 oligonucleotide that is capable of directing a decrease in the expression and/or level of a target gene or its gene product can comprise any optional additional chemical moiety, including but not limited to, a targeting moiety, a lipid moiety, a carbohydrate moiety, a bicyclic ketal, a GalNAc moiety, etc., described herein or known in the art.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any optional additional chemical moiety, including but not limited to, a targeting moiety, a lipid moiety, a carbohydrate moiety, a bicyclic ketal, a GalNAc moiety, etc., described herein or known in the art.

In some embodiments, an additional chemical moiety is conjugated to single-stranded RNAi agent.

Optional Additional Chemical Moiety Conjugated to an APOC3 Oligonucleotide: A Targeting Moiety

In some embodiments, a provided oligonucleotide composition further comprises a targeting moiety (e.g., a targeting compound, agent, ligand, or component). A targeting moiety can be either conjugated or not conjugated to a lipid or an APOC3 oligonucleotide or single-stranded RNAi agent. In some embodiments, a targeting moiety is conjugated to an APOC3 oligonucleotide or single-stranded RNAi agent. In some embodiments, an APOC3 oligonucleotide or single-stranded RNAi agent is conjugated to both a lipid and a targeting moiety. As described in here, in some embodiments, an APOC3 oligonucleotide or single-stranded RNAi agent is a provided oligonucleotide. Thus, in some embodiments, a provided oligonucleotide composition further comprises, besides a lipid and oligonucleotides, a target elements. Various targeting moieties can be used in accordance with the present disclosure, e.g., lipids, antibodies, peptides, carbohydrates, etc.

Targeting moieties can be incorporated into provided technologies through many types of methods in accordance with the present disclosure. In some embodiments, targeting moieties are physically mixed with provided oligonucleotides to form provided compositions. In some embodiments, targeting moieties are chemically conjugated with oligonucleotides.

In some embodiments, provided compositions comprise two or more targeting moieties.

In some embodiments, provided oligonucleotides comprise two or more conjugated targeting moieties. In some embodiments, the two or more conjugated targeting moieties are the same. In some embodiments, the two or more conjugated targeting moieties are different. In some embodiments, provided oligonucleotides comprise no more than one target component. In some embodiments, oligonucleotides of a provided composition comprise different types of conjugated targeting moieties. In some embodiments, oligonucleotides of a provided composition comprise the same type of targeting moieties.

In some embodiments, provided compositions comprise two or more targeting moieties.

In some embodiments, provided oligonucleotides capable of directing single-stranded RNA interference comprise two or more conjugated targeting moieties. In some embodiments, the two or more conjugated targeting moieties are the same. In some embodiments, the two or more conjugated targeting moieties are different. In some embodiments, provided oligonucleotides capable of directing single-stranded RNA interference comprise no more than one target component. In some embodiments, oligonucleotides of a provided composition comprise different types of conjugated targeting moieties. In some embodiments, oligonucleotides of a provided composition comprise the same type of targeting moieties.

Targeting moieties can be conjugated to oligonucleotides optionally through linkers.

Various types of linkers in the art can be utilized in accordance of the present disclosure. In some embodiments, a linker comprise a phosphate group, which can, for example, be used for conjugating targeting moieties through chemistry similar to those employed in oligonucleotide synthesis. In some embodiments, a linker comprises an amide, ester, or ether group. In some embodiments, a linker has the structure of -L-. Targeting moieties can be conjugated through either the same or different linkers compared to lipids.

Targeting moieties, optionally through linkers, can be conjugated to oligonucleotides at various suitable locations. In some embodiments, targeting moieties are conjugated through the 5′—OH group. In some embodiments, targeting moieties are conjugated through the 3′—OH group. In some embodiments, targeting moieties are conjugated through one or more sugar moieties. In some embodiments, targeting moieties are conjugated through one or more bases. In some embodiments, targeting moieties are incorporated through one or more internucleotidic linkages. In some embodiments, an APOC3 oligonucleotide may contain multiple conjugated targeting moieties which are independently conjugated through its 5′-OH, 3′-OH, sugar moieties, base moieties and/or internucleotidic linkages. Targeting moieties and lipids can be conjugated either at the same, neighboring and/or separated locations. In some embodiments, a target component is conjugated at one end of an APOC3 oligonucleotide, and a lipid is conjugated at the other end.

In some embodiments, a provided composition further comprises a targeting component or moiety. A targeting component can be either incorporated into (targeting moiety) or not incorporated into an APOC3 oligonucleotide. In some embodiments, a targeting component is a lipid. In some embodiments, a targeting component is a carbohydrate or a bicyclic ketal. In some embodiments, a targeting component is —R^{LD} as described in the present disclosure. In some embodiments, a targeting component is —R^{CD} as described in the present disclosure.

Targeting components can be incorporated into provided technologies through many types of methods in accordance with the present disclosure, for example, those described for lipids and carbohydrates. In some embodiments, targeting components are physically mixed with provided oligonucleotides to form provided compositions. In some embodiments, targeting components are chemically conjugated with oligonucleotide moieties.

In some embodiments, provided compositions comprise two or more targeting components. In some embodiments, provided oligonucleotides comprise two or more conjugated targeting components. In some embodiments, the two or more conjugated targeting components are the same. In some embodiments, the two or more conjugated targeting components are different. In some embodiments, provided oligonucleotides comprise no more than one targeting component. In some embodiments, oligonucleotides of a provided composition comprise different types of conjugated targeting components. In some embodiments, oligonucleotides of a provided composition comprise the same type of targeting components.

Targeting components can be conjugated to oligonucleotides optionally through linkers, for example, as described for lipids and carbohydrates. Various types of linkers in the art can be utilized in accordance of the present disclosure. In some embodiments, a linker comprise a phosphate group, which can, for example, be used for conjugating targeting components through chemistry similar to those employed in oligonucleotide synthesis. In some embodiments, a linker comprises an amide, ester, or ether group. In some embodiments, a linker has the structure of -L-. Targeting components can be conjugated through either the same or different linkers compared to lipids.

Targeting components, optionally through linkers, can be conjugated to oligonucleotides at various suitable locations. In some embodiments, targeting components are conjugated through the 5′-OH group. In some embodiments, targeting components are conjugated through the 3′—OH group. In some embodiments, targeting components are conjugated through one or more sugar moieties. In some embodiments, targeting components are conjugated through one or more bases. In some embodiments, targeting components are incorporated through one or more internucleotidic linkages. In some embodiments, an APOC3 oligonucleotide may contain multiple conjugated targeting components which are independently conjugated through its 5′-OH, 3′-OH, sugar moieties, base moieties and/or internucleotidic linkages. Targeting components and lipids can be conjugated either at the same, neighboring and/or separated locations. In some embodiments, a targeting component is conjugated at one end of an APOC3 oligonucleotide, and a lipid is conjugated at the other end.

In some embodiments, a targeting component interacts with a protein on the surface of targeted cells. In some embodiments, such interaction facilitates internalization into targeted cells. In some embodiments, a targeting component comprises a sugar moiety. In some embodiments, a targeting component comprises a polypeptide moiety. In some embodiments, a targeting component comprises an antibody. In some embodiments, a targeting component is an antibody. In some embodiments, a targeting component comprises an inhibitor. In some embodiments, a targeting component is a moiety from a small molecule inhibitor. In some embodiments, an inhibitor is an inhibitor of a protein on the surface of targeted cells. In some embodiments, an inhibitor is a carbonic anhydrase inhibitor. In some embodiments, an inhibitor is a carbonic anhydrase inhibitor expressed on the surface of target cells. In some embodiments, a carbonic anhydrase is I, II, III, IV, V, VI, VII, VIII, IX, X, XI, XII, XIII, XIV, XV or XVI. In some embodiments, a carbonic anhydrase is membrane bound. In some embodiments, a carbonic anhydrase is IV, IX, XII or XIV. In some embodiments, an inhibitor is for IV, IX, XII and/or XIV. In some embodiments, an inhibitor is a carbonic anhydrase III inhibitor. In some embodiments, an inhibitor is a carbonic anhydrase IV inhibitor. In some embodiments, an inhibitor is a carbonic anhydrase IX inhibitor. In some embodiments, an inhibitor is a carbonic anhydrase XII inhibitor. In some embodiments, an inhibitor is a carbonic anhydrase XIV inhibitor. In some embodiments, an inhibitor comprises or is a sulfonamide (e.g., those described in Supuran, Conn. Nature Rev Drug Discover 2008, 7, 168-181, which sulfonamides are incorporated herein by reference). In some embodiments, an inhibitor is a sulfonamide. In some embodiments, targeted cells are muscle cells.

In some embodiments, a targeting component is R^{TD}, wherein R^{TD} is R^{LD} or R^{CD} as described in the present disclosure.

In some embodiments, a targeting component is R^{LD} as defined and described in the present disclosure. In some embodiments, the present disclosure provides oligonucleotides comprising R^{LD}. In some embodiments, the present disclosure provides oligonucleotide compositions comprising oligonucleotides comprising R^{LD}. In some embodiments, the present disclosure provides oligonucleotide compositions comprising a first plurality of oligonucleotides comprising R^{LD}. In some embodiments, the present disclosure provides chirally controlled oligonucleotide compositions of oligonucleotides comprising R^{LD}.

In some embodiments, a targeting component is R^{CD} as defined and described in the present disclosure. In some embodiments, the present disclosure provides oligonucleotides comprising R^{CD}. In some embodiments, the present disclosure provides oligonucleotide compositions comprising oligonucleotides comprising R^{CD}. In some embodiments, the present disclosure provides oligonucleotide compositions comprising a first plurality of oligonucleotides comprising R^{CD}. In some embodiments, the present disclosure provides chirally controlled oligonucleotide compositions of oligonucleotides comprising R^{CD}.

In some embodiments, R^{TD} comprises or is

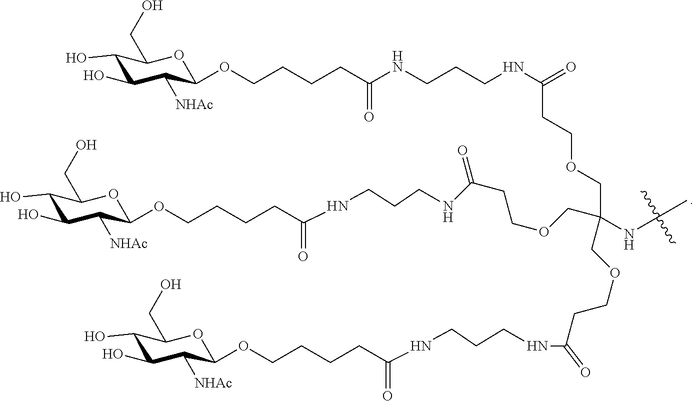

In some embodiments, R^{TD} comprises or is

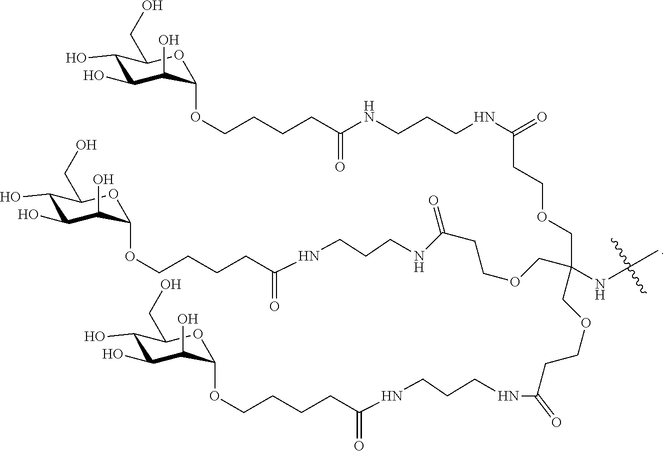

In some embodiments, R^{TD} comprises or is

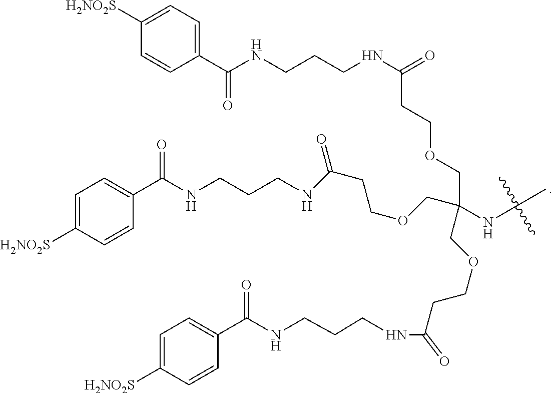 [SMILES: *NC(COCCC(=O)NCCCNC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, R^{TD} comprises or is

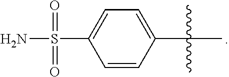 [SMILES: *c1ccc(S(N)(=O)=O)cc1]

In some embodiments, R^{TD} comprises or is

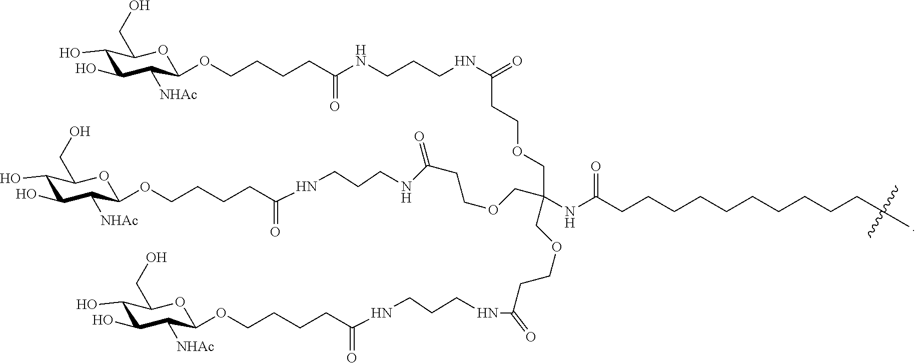

In some embodiments, R^{TD} comprises or is

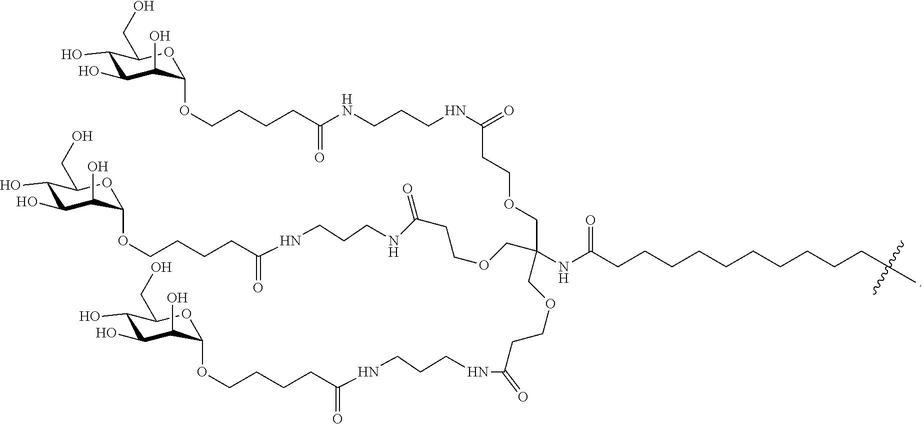

In some embodiments, R^{TD} comprises or is

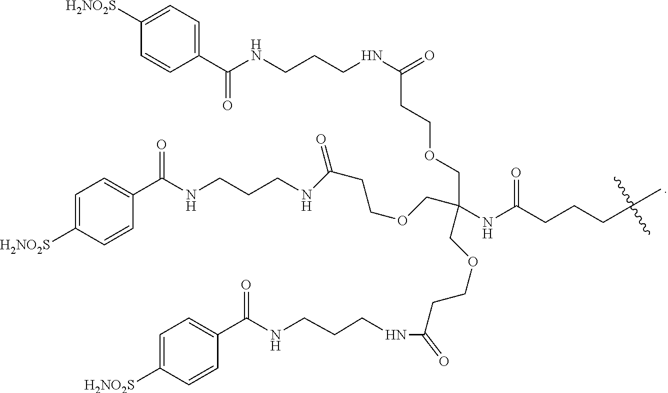 [SMILES: *CCCC(=O)NC(COCCC(=O)NCCCNC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, R^{TD} comprises or is

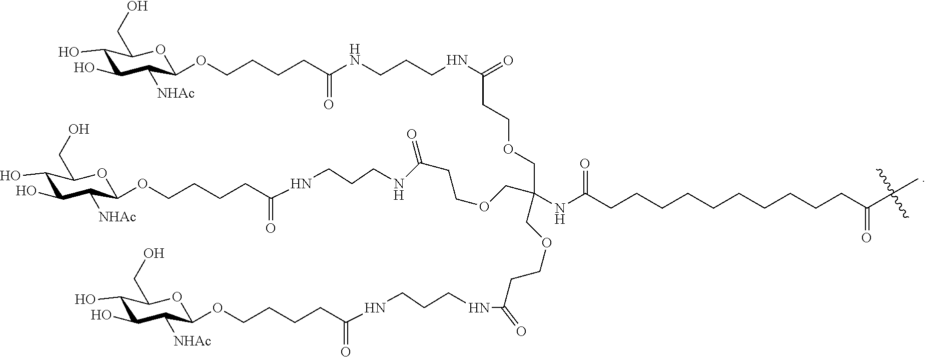

In some embodiments, R^{TD} comprises or is

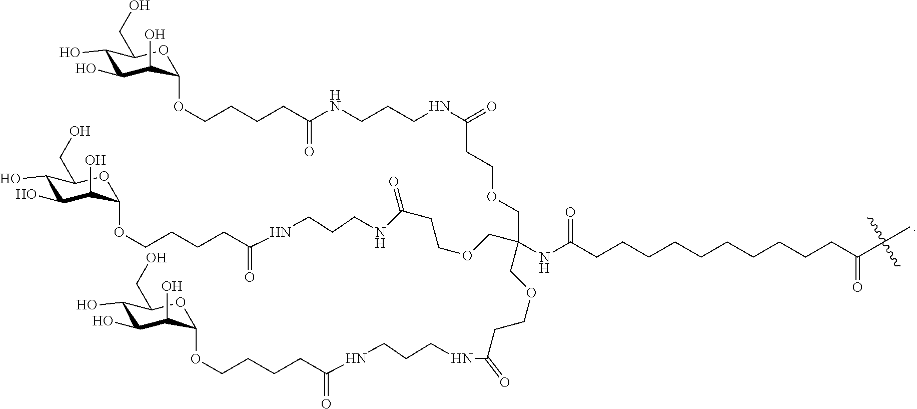

In some embodiments, R^{TD} comprises or is

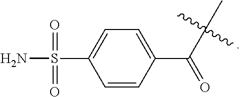 [SMILES: *C(=O)c1ccc(S(N)(=O)=O)cc1]

In some embodiments, R^{TD} comprises or is

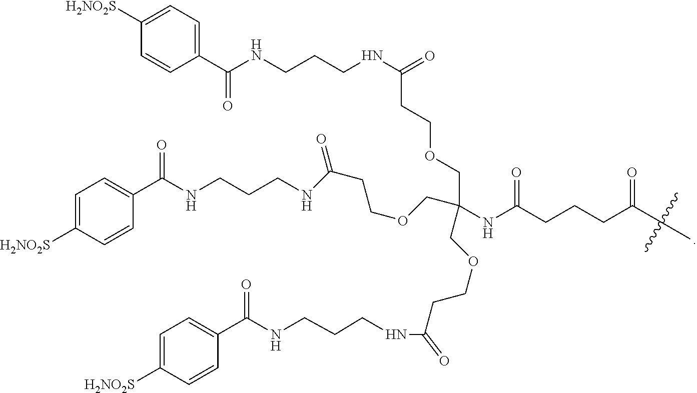 [SMILES: *C(=O)CCCC(=O)NC(COCCC(=O)NCCCNC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, R^{TD} comprises or is

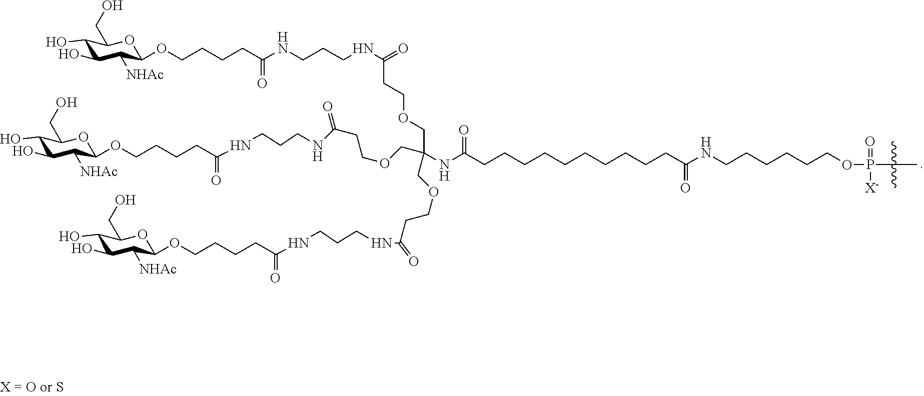

In some embodiments, R^{TD} comprises or is

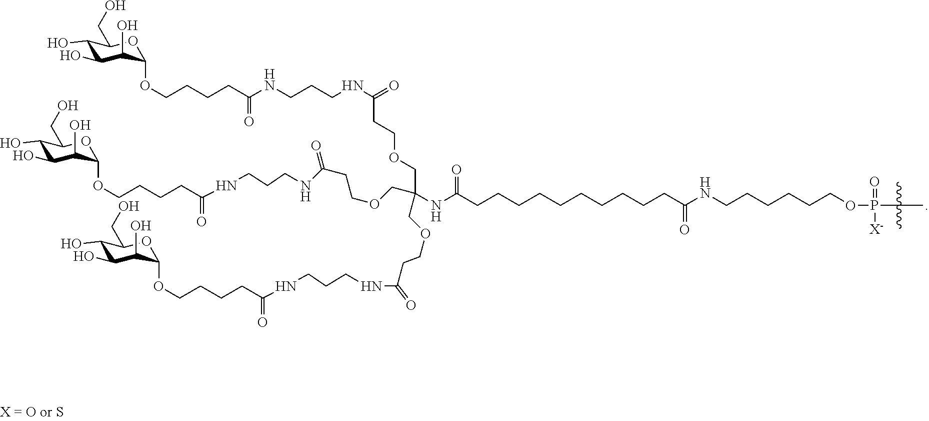

In some embodiments, R^{TD} comprises or is

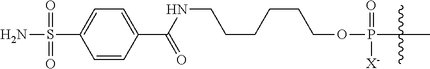

X═O or S. In some embodiments, R^{TD} comprises or is

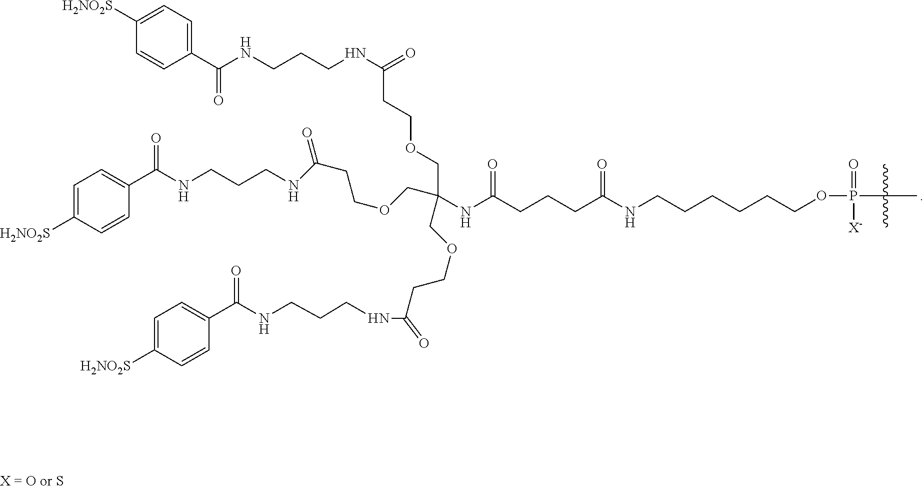

In some embodiments, R^{TD} is a targeting component that comprises or is a lipid moiety. In some embodiments, X is O. In some embodiments, X is S.

In some embodiments, the present disclosure provides technologies (e.g., reagents, methods, etc.) for conjugating various moieties to oligonucleotide moieties. In some embodiments, the present disclosure provides technologies for conjugating targeting component to oligonucleotide moieties. In some embodiments, the present disclosure provides acids comprising targeting components for conjugation, e.g., R^{LD}—COOH. In some embodiments, the present disclosure provides linkers for conjugation, e.g., L^{M}. A person having ordinary skill in the art understands that many known and widely practiced technologies can be utilized for conjugation with oligonucleotide moieties in accordance with the present disclosure. In some embodiments, a provided acid is

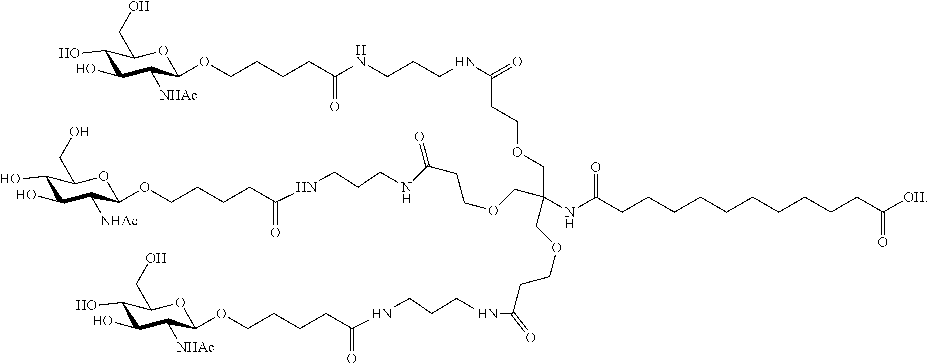

In some embodiments, a provided acid is

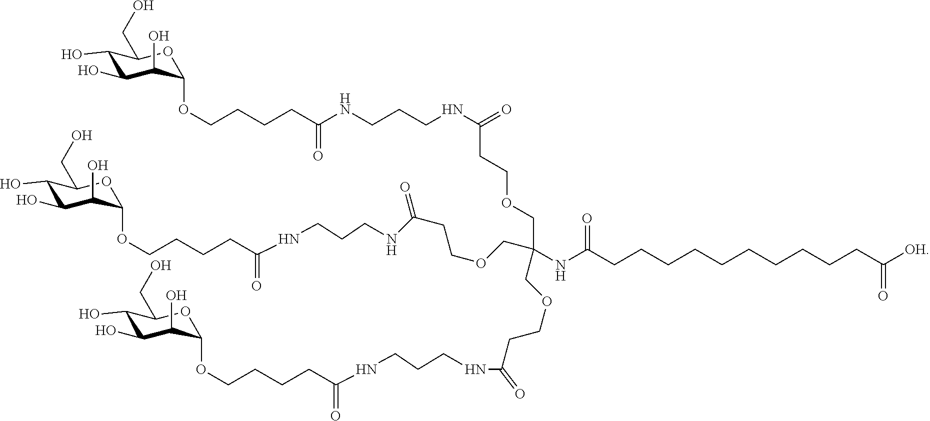

In some embodiments, a provided acid is

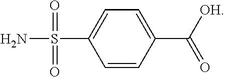 [SMILES: NS(=O)(=O)c1ccc(C(=O)O)cc1]

In some embodiments, a provided acid is

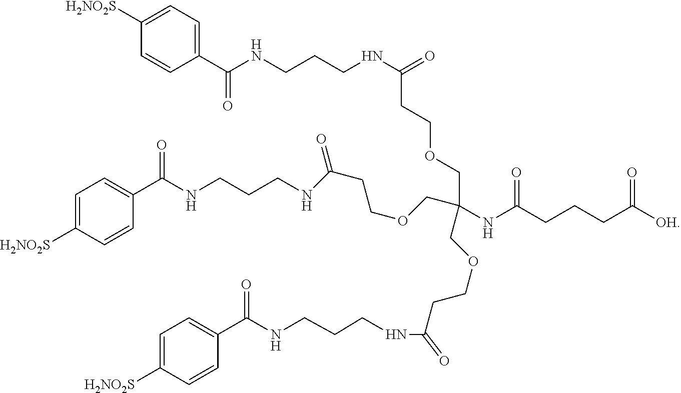 [SMILES: NS(=O)(=O)c1ccc(C(=O)NCCCNC(=O)CCOCC(COCCC(=O)NCCCNC(=O)c2ccc(S(N)(=O)=O)cc2)(COCCC(=O)NCCCNC(=O)c2ccc(S(N)(=O)=O)cc2)NC(=O)CCCC(=O)O)cc1]

In some embodiments, a provided acid is a fatty acid, which can provide a lipid moiety as a targeting component. In some embodiments, the present disclosure provides methods and reagents for preparing such acids.

In some embodiments, a targeting moiety is a lipid moiety, e.g., moiety of cholesterol or derivatives thereof (R^{TD}—H is an optionally substituted cholesterol or derivatives thereof).

In some embodiments, a targeting moiety is a peptide. In some embodiments, a targeting moiety is protein or a domain thereof. In some embodiments, a targeting moiety is antibody or a portion thereof.

Optional Additional Chemical Moieties Conjugated to an APOC3 Oligonucleotide: A Lipid Moiety

In some embodiments, provided oligonucleotides or oligonucleotide compositions further comprise one or more lipids or lipid moieties. In some embodiments, a lipid is a lipid moiety. In some embodiments, a lipid moiety is or comprises a lipid which is conjugated directly or indirectly to an APOC3 oligonucleotide. In some embodiments, lipid conjugation can achieve one or more unexpected, greatly improved properties (e.g., activities, toxicities, distribution, pharmacokinetics, etc.). As appreciated by a person having ordinary skill in the art, various carbohydrate moieties are described in the literature and can be utilized in accordance with the present disclosure.

Lipid moieties can be incorporated into oligonucleotides at various locations, for example, sugar units, internucleotidic linkage units, nucleobase units, etc., optionally through one or more bivalent or multivalent linkers (which can be used to connect two or more carbohydrate moieties to oligonucleotides). In some embodiments, the present disclosure provides technologies for lipid incorporation into oligonucleotides. In some embodiments, the present disclosure provides technologies for incorporating lipid moieties, optionally through one or more linkers, at nucleobase units, as an alternative and/or addition to incorporation at internucleotidic linkages and/or sugar units, thereby providing enormous flexibility and/or improved properties and/or activities. In some embodiments, a provided oligonucleotide comprises at least one lipid moiety, optionally through a linker, incorporated into the oligonucleotide at a nucleobase unit.

In some embodiments, provided oligonucleotides have the structure of:

A^{c}-[-L^{M}-(R^{D})ₐ]_{b}, or [(A^{c})ₐ-L^{M}]_{b}-R^{D},

wherein:

A^{c} is an APOC3 oligonucleotide chain ([H]_{b}-A^{c} is an APOC3 oligonucleotide);

a is 1-1000;

b is 1-1000;

each L^{M} is independently a linker; and

each R^{D} is independently R^{LD} or R^{CD}, P R^{CD} is an optionally substituted, linear or branched group selected from a C₁₋₁₀₀ aliphatic group and a C₁₋₁₀₀ heteroaliphatic group having 1-30 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy;

R^{LD} is an optionally substituted, linear or branched group selected from a C₁₋₁₀₀ aliphatic group wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)2-, —S(O)2N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy;

L^{M} is a covalent bond, or a bivalent or multivalent, optionally substituted, linear or branched group selected from a C₁₋₁₀₀ aliphatic group and a C₁₋₁₀₀ heteroaliphatic group having 1-30 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy^{L};

Cy^{L} is an optionally substituted tetravalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus, boron and silicon;

each R′ is independently —R, —C(O)R, —C(O)OR, or —S(O)₂R; and

each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or

two R groups are optionally and independently taken together to form a covalent bond, or:

two or more R groups on the same atom are optionally and independently taken together with the atom to form an optionally substituted, 3-30 membered monocyclic, bicyclic or polycyclic ring having, in addition to the atom, 0-10 heteroatoms independently selected from oxygen, nitrogen, sulfur, phosphorus and silicon; or

two or more R groups on two or more atoms are optionally and independently taken together with their intervening atoms to form an optionally substituted, 3-30 membered monocyclic, bicyclic or polycyclic ring having, in addition to the intervening atoms, 0-10 heteroatoms independently selected from oxygen, nitrogen, sulfur, phosphorus and silicon.

In some embodiments, R^{CD} is a carbohydrate moiety or a bicyclic ketal. In some embodiments, R^{CD} comprises at least one monosaccharide, disaccharide, or polysaccharide units. In some embodiments, R^{CD} comprises at least one GalNAc moiety or a derivative thereof.

In some embodiments, R^{LD} is a lipid moiety. In some embodiments, R^{LD} comprises one or more optionally substituted C₆₋₂₀ aliphatic chain. In some embodiments, R^{LD} comprises one or more unsubstituted C₆₋₂₀ aliphatic chain.

In some embodiments, at least one L^{M} is directly bound to a sugar unit of a provided oligonucleotide. In some embodiments, a L^{M} directly binds to a sugar unit incorporates a lipid moiety into an APOC3 oligonucleotide. In some embodiments, a L^{M} directly binds to a sugar unit incorporates a carbohydrate moiety into an APOC3 oligonucleotide. In some embodiments, a L^{M} directly binds to a sugar unit incorporates a R^{LD} group into an APOC3 oligonucleotide. In some embodiments, a L^{M} directly binds to a sugar unit incorporates a R^{CD} group into an APOC3 oligonucleotide.

In some embodiments, at least one L^{M} is directly bound to an internucleotidic linkage unit of a provided oligonucleotide. In some embodiments, a L^{M} directly binds to an internucleotidic linkage unit incorporates a lipid moiety into an APOC3 oligonucleotide. In some embodiments, a L^{M} directly binds to an internucleotidic linkage unit incorporates a carbohydrate moiety into an APOC3 oligonucleotide. In some embodiments, a L^{M} directly binds to an internucleotidic linkage unit incorporates a R^{LD} group into an APOC3 oligonucleotide. In some embodiments, a L^{M} directly binds to an internucleotidic linkage unit incorporates a R^{CD} group into an APOC3 oligonucleotide.

In some embodiments, at least one L^{M} is directly bound to a nucleobase unit of a provided oligonucleotide. In some embodiments, a L^{M} directly binds to a nucleobase unit incorporates a lipid moiety into an APOC3 oligonucleotide. In some embodiments, a L^{M} directly binds to a nucleobase unit incorporates a carbohydrate moiety into an APOC3 oligonucleotide. In some embodiments, a L^{M} directly binds to a nucleobase unit incorporates a R^{LD} group into an APOC3 oligonucleotide. In some embodiments, a L^{M} directly binds to a nucleobase unit incorporates a R^{CD} group into an APOC3 oligonucleotide.

In some embodiments, [H]_{b}-A^{c} is an APOC3 oligonucleotide described in the present disclosure.

In some embodiments, incorporation of a lipid into a provided oligonucleotide improves distribution and/or pharmacokinetics. In some embodiments, incorporation of a lipid into a provided oligonucleotide improves one or more measurement of pharmacokinetics selected from: Cₘₐₓ, peak plasma concentration of a drug after administration; tₘₐₓ, time to reach Cₘₐₓ; Cₘᵢₙ, lowest (trough) concentration that a drug reaches before the next dose is administered; elimination half-life, the time required for the concentration of the drug to reach half of its original value; elimination rate constant, rate at which a drug is removed from the body; area under the curve, integral of the concentration-time curve (after a single dose or in steady state); and clearance, volume of plasma cleared of the drug per unit time. Without being bound to any particular theory, this disclosure notes that optimization of a pharmacokinetic characteristic such as half-life can be distinguished from maximization. In some embodiments, in general, it may be desirable for a particular drug to have a half-life sufficient to allow performance of its desired function, but short enough to minimize off-target effects and other toxicity. In some embodiments, an optimized half-life is long enough to allow activity while minimizing toxicity; a prolonged or maximized half-life may be undesirable.

In some embodiments, provided oligonucleotide compositions further comprise one or more lipids. In some embodiments, provided oligonucleotide compositions further comprise one or more fatty acids. In some embodiments, the lipids can be incorporated into provided oligonucleotides in the compositions. In some embodiments, two or more same or different lipids can be incorporated into one oligonucleotide, through either the same or differently chemistry and/or locations.

Many lipids can be utilized in provided technologies in accordance with the present disclosure. In some embodiments, a lipid comprises an R^{LD} group. In some embodiments, R^{LD} is an optionally substituted, C₁₀-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and —Cy-. In some embodiments, R^{LD} is an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and —Cy-. In some embodiments, R^{LD} is a hydrocarbon group consisting of carbon and hydrogen atoms. In some embodiments, —Cy— is an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus, boron and silicon.

In some embodiments, R^{LD} is an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and —Cy-. In some embodiments, R^{LD} is an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and —Cy-. In some embodiments, R^{LD} is a hydrocarbon group consisting carbon and hydrogen atoms.

In some embodiments, R^{LD} is an optionally substituted, C₁₀-C₄₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and —Cy-. In some embodiments, R^{LD} is an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, and —Cy-. In some embodiments, R^{LD} is a hydrocarbon group consisting carbon and hydrogen atoms.

The aliphatic group of R^{LD} can be a variety of suitable length. In some embodiments, it is C₁₀-C₈₀. In some embodiments, it is C₁₀-C₇₅. In some embodiments, it is C₁₀-C₇₀. In some embodiments, it is C₁₀-C₆₅. In some embodiments, it is C₁₀-C₆₀. In some embodiments, it is C₁₀-C₅₀. In some embodiments, it is C₁₀-C₄₀. In some embodiments, it is C₁₀-C₃₅. In some embodiments, it is C₁₀-C₃₀. In some embodiments, it is C₁₀-C₂₅. In some embodiments, it is C₁₀-C₂₄. In some embodiments, it is C₁₀-C₂₃. In some embodiments, it is C₁₀-C₂₂. In some embodiments, it is C₁₀-C₂₁. In some embodiments, it is C₁₂-C₂₂. In some embodiments, it is C₁₃-C₂₂. In some embodiments, it is C₁₄-C₂₂. In some embodiments, it is C₁₅-C₂₂. In some embodiments, it is C₁₆-C₂₂. In some embodiments, it is C₁₇-C₂₂. In some embodiments, it is C₁₈-C₂₂. In some embodiments, it is C₁₀-C₂₀. In some embodiments, the lower end of the range is C₁₀, C₁₁, C₁₂, C₁₃, C₁₄, C₁₅, C₁₆, C₁₇, or C₁₈. In some embodiments, the higher end of the range is C₁₈, C₁₉, C₂₀, C₂₁, C₂₂, C₂₃, C₂₄, C₂₅, C₂₆, C₂₇, C₂₈, C₂₉, C₃₀, C₃₅, C₄₀, C₄₅, C₅₀, C₅₅, or C₆₀. In some embodiments, it is C₁₀. In some embodiments, it is C₁₁. In some embodiments, it is C₁₂. In some embodiments, it is C₁₃. In some embodiments, it is C₁₄. In some embodiments, it is C₁₅. In some embodiments, it is C₁₆. In some embodiments, it is C₁₇. In some embodiments, it is C₁₈. In some embodiments, it is C₁₉. In some embodiments, it is C₂₀. In some embodiments, it is C₂₁. In some embodiments, it is C₂₂. In some embodiments, it is C₂₃. In some embodiments, it is C₂₄. In some embodiments, it is C₂₅. In some embodiments, it is C₃₀. In some embodiments, it is C₃₅. In some embodiments, it is C₄₀. In some embodiments, it is C₄₅. In some embodiments, it is C₅₀. In some embodiments, it is C₅₅. In some embodiments, it is C₆₀.

In some embodiments, a lipid comprises no more than one R^{LD} group. In some embodiments, a lipid comprises two or more R^{LD} groups.

In some embodiments, a lipid is conjugated to a biologically active agent, optionally through a linker, as a moiety comprising an R^{LD} group. In some embodiments, a lipid is conjugated to a biologically active agent, optionally through a linker, as a moiety comprising no more than one R^{LD} group. In some embodiments, a lipid is conjugated to a biologically active agent, optionally through a linker, as an R^{LD} group. In some embodiments, a lipid is conjugated to a biologically active agent, optionally through a linker, as a moiety comprising two or more R^{LD} groups.

In some embodiments, R^{LD} is an optionally substituted, C₁₀-C₄₀ saturated or partially unsaturated, aliphatic chain. In some embodiments, a lipid comprises an optionally substituted C₁₀-C₄₀ saturated or partially unsaturated, aliphatic chain.

In some embodiments, R^{LD} is an optionally substituted C₁₀-C₄₀ linear, saturated or partially unsaturated, aliphatic chain. In some embodiments, a lipid comprises an optionally substituted C₁₀-C₄₀ linear, saturated or partially unsaturated, aliphatic chain.

In some embodiments, R^{LD} is a C₁₀-C₄₀ linear, saturated or partially unsaturated, aliphatic chain, optionally substituted with one or more C₁₋₄ aliphatic groups. In some embodiments, a lipid comprises a C₁₀-C₄₀ linear, saturated or partially unsaturated, aliphatic chain, optionally substituted with one or more C₁₋₄ aliphatic groups. In some embodiments, R^{LD} is a C₁₀-C₄₀ linear, saturated or partially unsaturated, aliphatic chain, optionally substituted with one or more C₁₋₂ aliphatic groups. In some embodiments, a lipid comprises a C₁₀-C₄₀ linear, saturated or partially unsaturated, aliphatic chain, optionally substituted with one or more C₁₋₂ aliphatic groups. In some embodiments, R^{LD} is a C₁₀-C₄₀ linear, saturated or partially unsaturated, aliphatic chain, optionally substituted with one or more methyl groups. In some embodiments, a lipid comprises a C₁₀-C₄₀ linear, saturated or partially unsaturated, aliphatic chain, optionally substituted with one or more methyl groups.

In some embodiments, R^{LD} is an unsubstituted C₁₀-C₄₀ linear, saturated or partially unsaturated, aliphatic chain. In some embodiments, a lipid comprises an unsubstituted C₁₀-C₄₀ linear, saturated or partially unsaturated, aliphatic chain.

In some embodiments, a lipid comprises no more than one optionally substituted C₁₀-C₄₀ linear, saturated or partially unsaturated, aliphatic chain. In some embodiments, a lipid comprises two or more optionally substituted C₁₀-C₄₀ linear, saturated or partially unsaturated, aliphatic chain.

In some embodiments, R^{LD} is an optionally substituted, C₁₀-C₆₀ saturated or partially unsaturated, aliphatic chain. In some embodiments, a lipid comprises an optionally substituted C₁₀-C₆₀ saturated or partially unsaturated, aliphatic chain.

In some embodiments, R^{LD} is an optionally substituted C₁₀-C₆₀ linear, saturated or partially unsaturated, aliphatic chain. In some embodiments, a lipid comprises an optionally substituted C₁₀-C₆₀ linear, saturated or partially unsaturated, aliphatic chain.

In some embodiments, R^{LD} is a C₁₀-C₆₀ linear, saturated or partially unsaturated, aliphatic chain, optionally substituted with one or more C₁₋₄ aliphatic groups. In some embodiments, a lipid comprises a C₁₀-C₆₀ linear, saturated or partially unsaturated, aliphatic chain, optionally substituted with one or more C₁₋₄ aliphatic groups. In some embodiments, R^{LD} is a C₁₀-C₆₀ linear, saturated or partially unsaturated, aliphatic chain, optionally substituted with one or more C₁₋₂ aliphatic groups. In some embodiments, a lipid comprises a C₁₀-C₆₀ linear, saturated or partially unsaturated, aliphatic chain, optionally substituted with one or more C₁₋₂ aliphatic groups. In some embodiments, R^{LD} is a C₁₀-C₆₀ linear, saturated or partially unsaturated, aliphatic chain, optionally substituted with one or more methyl groups. In some embodiments, a lipid comprises a C₁₀-C₆₀ linear, saturated or partially unsaturated, aliphatic chain, optionally substituted with one or more methyl groups.

In some embodiments, R^{LD} is an unsubstituted C₁₀-C₆₀ linear, saturated or partially unsaturated, aliphatic chain. In some embodiments, a lipid comprises an unsubstituted C₁₀-C₆₀ linear, saturated or partially unsaturated, aliphatic chain.

In some embodiments, a lipid comprises no more than one optionally substituted C₁₀-C₆₀ linear, saturated or partially unsaturated, aliphatic chain. In some embodiments, a lipid comprises two or more optionally substituted C₁₀-C₆₀ linear, saturated or partially unsaturated, aliphatic chain.

In some embodiments, R^{LD} is an optionally substituted, C₁₀-C₈₀ saturated or partially unsaturated, aliphatic chain. In some embodiments, a lipid comprises an optionally substituted C₁₀-C₈₀ saturated or partially unsaturated, aliphatic chain.

In some embodiments, R^{LD} is an optionally substituted C₁₀-C₈₀ linear, saturated or partially unsaturated, aliphatic chain. In some embodiments, a lipid comprises an optionally substituted C₁₀-C₈₀ linear, saturated or partially unsaturated, aliphatic chain.

In some embodiments, R^{LD} is a C₁₀-C₈₀ linear, saturated or partially unsaturated, aliphatic chain, optionally substituted with one or more C₁₋₄ aliphatic groups. In some embodiments, a lipid comprises a C₁₀-C₈₀ linear, saturated or partially unsaturated, aliphatic chain, optionally substituted with one or more C₁₋₄ aliphatic groups. In some embodiments, R^{LD} is a C₁₀-C₈₀ linear, saturated or partially unsaturated, aliphatic chain, optionally substituted with one or more C₁₋₂ aliphatic groups. In some embodiments, a lipid comprises a C₁₀-C₈₀ linear, saturated or partially unsaturated, aliphatic chain, optionally substituted with one or more C₁₋₂ aliphatic groups. In some embodiments, R^{LD} is a C₁₀-C₈₀ linear, saturated or partially unsaturated, aliphatic chain, optionally substituted with one or more methyl groups. In some embodiments, a lipid comprises a C₁₀-C₈₀ linear, saturated or partially unsaturated, aliphatic chain, optionally substituted with one or more methyl groups.

In some embodiments, R^{LD} is an unsubstituted C₁₀-C₈₀ linear, saturated or partially unsaturated, aliphatic chain. In some embodiments, a lipid comprises an unsubstituted C₁₀-C₈₀ linear, saturated or partially unsaturated, aliphatic chain.

In some embodiments, a lipid comprises no more than one optionally substituted C₁₀-C₈₀ linear, saturated or partially unsaturated, aliphatic chain. In some embodiments, a lipid comprises two or more optionally substituted C₁₀-C₈₀ linear, saturated or partially unsaturated, aliphatic chain.

In some embodiments, R^{LD} is or comprises a C₁₀ saturated linear aliphatic chain. In some embodiments, R^{LD} is or comprises a C₁₀ partially unsaturated linear aliphatic chain. In some embodiments, R^{LD} is or comprises a C₁₁ saturated linear aliphatic chain. In some embodiments, R^{LD} is or comprises a C₁₁ partially unsaturated linear aliphatic chain. In some embodiments, R^{LD} is or comprises a Cu saturated linear aliphatic chain. In some embodiments, R^{LD} is or comprises a C₁₂ partially unsaturated linear aliphatic chain. In some embodiments, R^{LD} is or comprises a C₁₃ saturated linear aliphatic chain. In some embodiments, R^{LD} is or comprises a C₁₃ partially unsaturated linear aliphatic chain. In some embodiments, R^{LD} is or comprises a C₁₋₄ saturated linear aliphatic chain. In some embodiments, R^{LD} is or comprises a C₁₋₄ partially unsaturated linear aliphatic chain. In some embodiments, R^{LD} is or comprises a C₁₅ saturated linear aliphatic chain. In some embodiments, R^{LD} is or comprises a C₁₅ partially unsaturated linear aliphatic chain. In some embodiments, R^{LD} is or comprises a C₁₆ saturated linear aliphatic chain. In some embodiments, R^{LD} is or comprises a C₁₆ partially unsaturated linear aliphatic chain. In some embodiments, R^{LD} is or comprises a C₁₇ saturated linear aliphatic chain. In some embodiments, R^{LD} is or comprises a C₁₇ partially unsaturated linear aliphatic chain. In some embodiments, R^{LD} is or comprises a C₁₈ saturated linear aliphatic chain. In some embodiments, R^{LD} is or comprises a C₁₈ partially unsaturated linear aliphatic chain. In some embodiments, R^{LD} is or comprises a C₁₉ saturated linear aliphatic chain. In some embodiments, R^{LD} is or comprises a C₁₉ partially unsaturated linear aliphatic chain. In some embodiments, R^{LD} is or comprises a C₂₀ saturated linear aliphatic chain. In some embodiments, R^{LD} is or comprises a C₂₀ partially unsaturated linear aliphatic chain. In some embodiments, R^{LD} is or comprises a C₂₁ saturated linear aliphatic chain. In some embodiments, R^{LD} is or comprises a C₂₁ partially unsaturated linear aliphatic chain. In some embodiments, R^{LD} is or comprises a C₂₂ saturated linear aliphatic chain. In some embodiments, R^{LD} is or comprises a C₂₂ partially unsaturated linear aliphatic chain. In some embodiments, R^{LD} is or comprises a C₂₃ saturated linear aliphatic chain. In some embodiments, R^{LD} is or comprises a C₂₃ partially unsaturated linear aliphatic chain. In some embodiments, R^{LD} is or comprises a C₂₄ saturated linear aliphatic chain. In some embodiments, R^{LD} is or comprises a C₂₄ partially unsaturated linear aliphatic chain. In some embodiments, R^{LD} is or comprises a C₂₅ saturated linear aliphatic chain. In some embodiments, R^{LD} is or comprises a C₂₅ partially unsaturated linear aliphatic chain. In some embodiments, R^{LD} is or comprises a C₂₆ saturated linear aliphatic chain. In some embodiments, R^{LD} is or comprises a C₂₆ partially unsaturated linear aliphatic chain. In some embodiments, R^{LD} is or comprises a C₂₇ saturated linear aliphatic chain. In some embodiments, R^{LD} is or comprises a C₂₇ partially unsaturated linear aliphatic chain. In some embodiments, R^{LD} is or comprises a C₂₈ saturated linear aliphatic chain. In some embodiments, R^{LD} is or comprises a C₂₈ partially unsaturated linear aliphatic chain. In some embodiments, R^{LD} is or comprises a C₂₉ saturated linear aliphatic chain. In some embodiments, R^{LD} is or comprises a C₂₉ partially unsaturated linear aliphatic chain. In some embodiments, R^{LD} is or comprises a C₃₀ saturated linear aliphatic chain. In some embodiments, R^{LD} is or comprises a C₃₀ partially unsaturated linear aliphatic chain.

In some embodiments, R^{LD} is derived from cholesterol or a derivatives thereof, e.g., R^{LD}—H is optionally substituted cholesterol or a derivative thereof.

In some embodiments, a lipid has the structure of R^{LD}—OH. In some embodiments, a lipid has the structure of R^{LD}—C(O)OH. In some embodiments, R^{LD} is

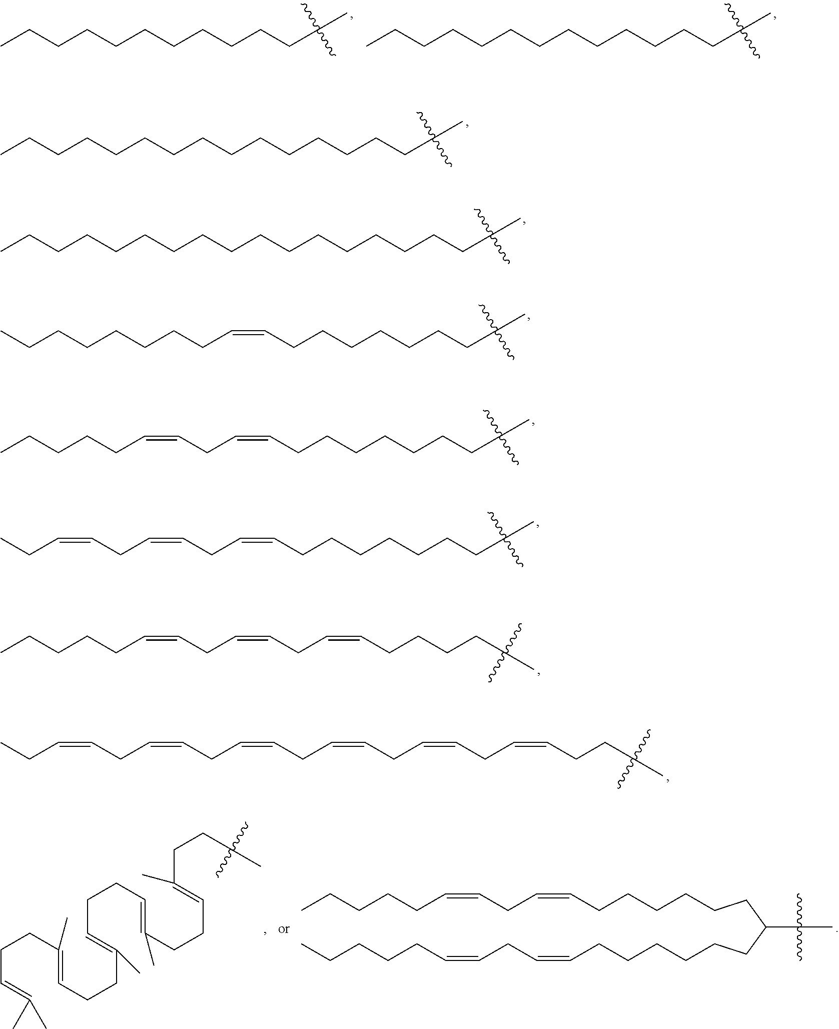

In some embodiments, a lipid is lauric acid, myristic acid, palmitic acid, stearic acid, oleic acid, linoleic acid, alpha-linolenic acid, gamma-linolenic acid, docosahexaenoic acid (DHA or cis-DHA), turbinaric acid, arachidonic acid, and dilinoleyl. In some embodiments, a lipid is lauric acid, myristic acid, palmitic acid, stearic acid, oleic acid, linoleic acid, alpha-linolenic acid, gamma-linolenic acid, docosahexaenoic acid (DHA or cis-DHA), turbinaric acid and dilinoleyl. In some embodiments, a lipid has a structure of:

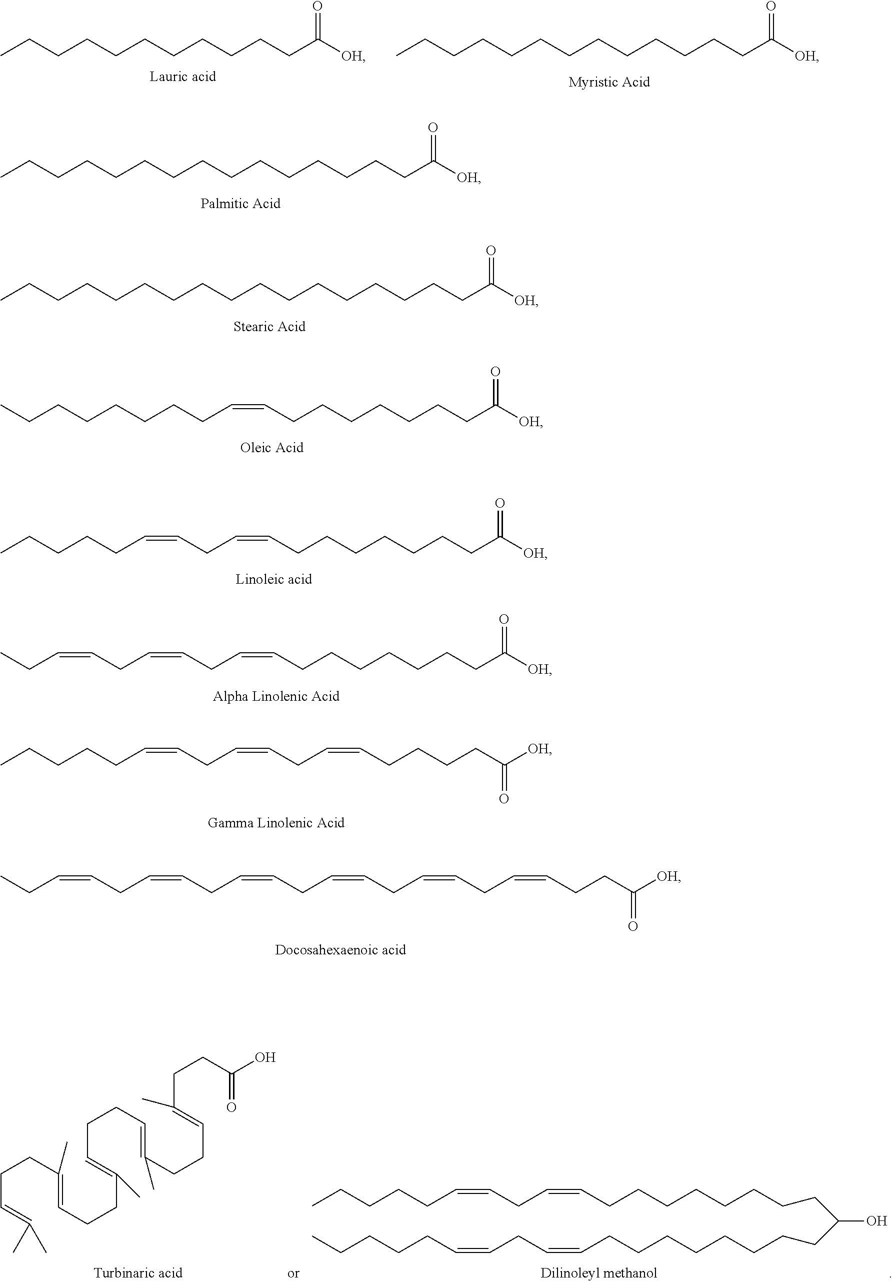

In some embodiments, a lipid is, comprises or consists of any of: an at least partially hydrophobic or amphiphilic molecule, a phospholipid, a triglyceride, a diglyceride, a monoglyceride, a fat-soluble vitamin, a sterol, a fat and a wax. In some embodiments, a lipid is any of: a fatty acid, glycerolipid, glycerophospholipid, sphingolipid, sterol lipid, prenol lipid, saccharolipid, polyketide, and other molecule.

Lipids can be incorporated into provided technologies through many types of methods in accordance with the present disclosure. In some embodiments, lipids are physically mixed with provided oligonucleotides to form provided compositions. In some embodiments, lipids are chemically conjugated with oligonucleotide moieties.

In some embodiments, provided compositions comprise two or more lipids. In some embodiments, provided oligonucleotides comprise two or more conjugated lipids. In some embodiments, the two or more conjugated lipids are the same. In some embodiments, the two or more conjugated lipids are different. In some embodiments, provided oligonucleotides comprise no more than one lipid. In some embodiments, oligonucleotides of a provided composition comprise different types of conjugated lipids. In some embodiments, oligonucleotides of a provided composition comprise the same type of lipids.

Lipids can be conjugated to oligonucleotides optionally through linkers. Various types of linkers in the art can be utilized in accordance of the present disclosure. In some embodiments, a linker is L^{M} as described in the present disclosure. In some embodiments, a linker comprise a phosphate group, which can, for example, be used for conjugating lipids through chemistry similar to those employed in oligonucleotide synthesis. In some embodiments, a linker comprises an amide, ester, or ether group.

In some embodiments, a linker has the structure of -L^{M}-. In some embodiments, L^{M} is L^{D}. In some embodiments, L^{D} is T^{D} having the structure of

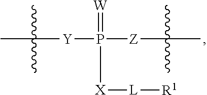

wherein each variable is independently as defined and described. In some embodiments, T^{D} has the structure of formula I. In some embodiments, T^{D} with the 5′-O— of an APOC3 oligonucleotide moiety form a phosphorothioate linkage (—OP(O)(S⁻)O—). In some embodiments, T^{D} with the 5′-O— of an APOC3 oligonucleotide moiety form an Sp phosphorothioate linkage. In some embodiments, T^{D} with the 5′-O— of an APOC3 oligonucleotide moiety form an Rp phosphorothioate linkage. In some embodiments, T^{D} with the 5′-O— of an APOC3 oligonucleotide moiety form a phosphate linkage (—OP(O)(O⁻)O—). In some embodiments, T^{D} with the 5′-O— of an APOC3 oligonucleotide moiety form a phosphorodithioate linkage. In some embodiments, L^{D} is -L-T^{D}-. In some embodiments, Y connects to -L- and —Z— is a covalent bond, so that P directly connects to a hydroxyl group of the oligonucleotide moiety. In some embodiments, P connects to the 5′-end hydroxyl (5′-O—) to form a phosphate group (natural phosphate linkage) or phosphorothioate group (phosphorothioate linkage). In some embodiments, the phosphorothioate linkage is chirally controlled and can be either Rp or Sp. Unless otherwise specified, chiral centers in the linkers (e.g., P in T^{D}) can be either stereorandom or chirally controlled, and they are not considered as part of the backbone chiral centers, e.g., for determining whether a composition is chirally controlled. In some embodiments, L^{D} is —NH—(CH₂)₆-T^{D}-. In some embodiments, L^{D} is —C(O)—NH—(CH₂)₆-T^{D}-.

In some embodiments, a linker has the structure of -L-. In some embodiments, after conjugation to oligonucleotides, a lipid forms a moiety having the structure of -L-R^{LD}, wherein each of L and R^{LD} is independently as defined and described herein.

In some embodiments, -L- comprises a bivalent aliphatic chain. In some embodiments, -L- comprises a phosphate group. In some embodiments, -L- comprises a phosphorothioate group. In some embodiments, -L- has the structure of —C(O)NH—(CH₂)₆—OP(═O)(S⁻)—. In some embodiments, -L- has the structure of —C(O)NH—(CH₂)₆—OP(═O)(O⁻)—.

Lipids, optionally through linkers, can be incorporated into oligonucleotides at various suitable locations. In some embodiments, lipids are conjugated through the 5′—OH group. In some embodiments, lipids are conjugated through the 3′—OH group. In some embodiments, lipids are conjugated through one or more sugar moieties. In some embodiments, lipids are conjugated through one or more bases. In some embodiments, lipids are incorporated through one or more internucleotidic linkages. In some embodiments, an APOC3 oligonucleotide may contain multiple conjugated lipids which are independently conjugated through its 5′-OH, 3′-OH, sugar moieties, base moieties and/or internucleotidic linkages.

In some embodiments, a linker is a moiety that connects two parts of a composition; as a non-limiting example, a linker physically connects an APOC3 oligonucleotide moiety to a lipid. Non-limiting examples of suitable linkers include: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; a linker comprising at least one peptide-based cleavage group.

In some embodiments, a lipid is conjugated to an active compound optionally through a linker moiety. A person having ordinary skill in the art appreciates that various technologies can be utilized to conjugate lipids to active compound in accordance with the present disclosure. For example, for lipids comprising carboxyl groups, such lipids can be conjugated through the carboxyl groups. In some embodiments, a lipid is conjugated through a linker having the structure of -L-, wherein L is as defined and described in formula I. In some embodiments, L comprises a phosphate diester or modified phosphate diester moiety. In some embodiments, a compound formed by lipid conjugation has the structure of (R^{LD}-L-)ₐ-(active compound), wherein a is 1 or an integer greater than 1, and each of R^{LD} and L is independently as defined and described herein. In some embodiments, a is 1. In some embodiments, a is greater than 1. In some embodiments, a is 1-50. In some embodiments, an active compound is an APOC3 oligonucleotide. For example, in some embodiments, a conjugate has any of the following structures:

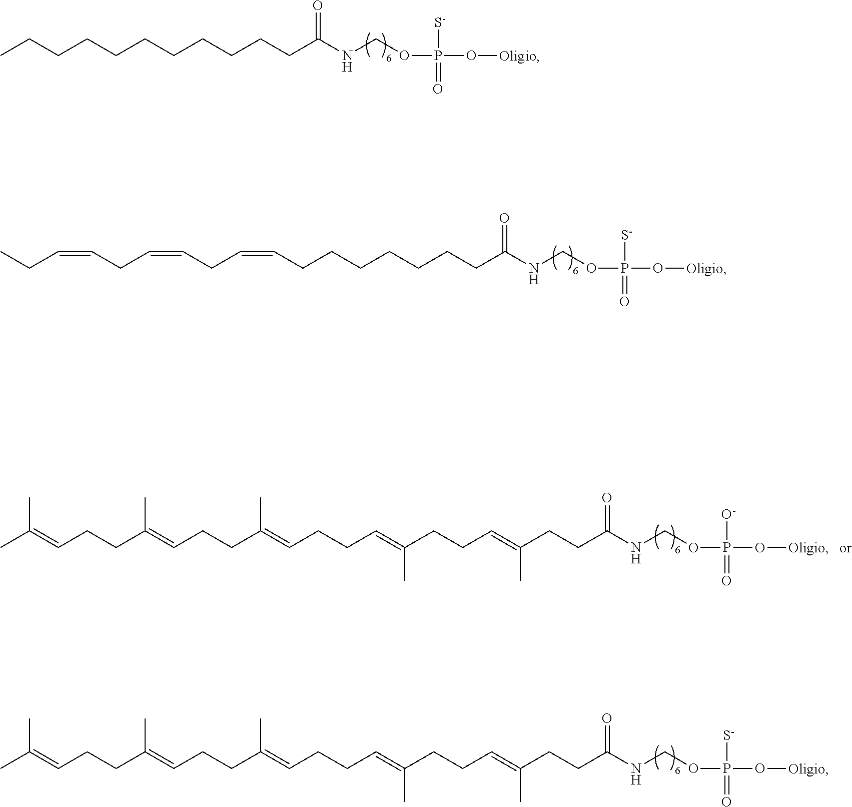

wherein Oligo indicates an oligonucleotide.

In some embodiments, a linker is selected from: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; and a linker comprising at least one peptide-based cleavage group. In some embodiments, a linker, e.g., L^{M}, has the structure of -L^{LD}-. In some embodiments, a linker, e.g., L^{M}, has the structure of -L-. In some embodiments, a linker comprises a linkage of formula I. In some embodiments, a linker is —C(O)NH—(CH₂)₆-L^{I}-, wherein L^{I} has the structure of formula I as described herein. In some embodiments, a linker is —C(O)NH—(CH₂)₆—O—P(═O)(SR¹)—O—. In some embodiments, R¹ is —H, and a linker is —C(O)NH—(CH₂)₆—O—P(═O)(SH)—O—, in some conditions, e.g., certain pH, —C(O)NH—(CH₂)₆—O—P(═O)(S⁻)—O—. In some embodiments, a linker is —C(O)NH—(CH₂)₆—O—P(═S)(SR¹)—O—. In some embodiments, R¹ is —H, and a linker is —C(O)NH—(CH₂)₆—O—P(═S)(SH)—O—, in some conditions, e.g., certain pH, —C(O)NH—(CH₂)₆—O—P(═S)(S⁻)—O—. In some embodiments, a linker is —C(O)NH—(CH₂)₆—O—P(═S)(OR¹)—O—, wherein R¹ is —CH₂CH₂CN. In some embodiments, a linker is —C(O)NH—(CH₂)₆—O—P(═S)(SR¹)—O—, wherein R¹ is —CH₂CH₂CN. In some embodiments, a provided oligonucleotide is coupled with a linker and forms a structure of H-linker-oligonucleotide. In some embodiments, a provided oligonucleotide is conjugated to a lipid and forms the structure of lipid-linker-oligonucleotide, e.g., R^{LD}-L^{LD}-oligonucleotide. In some embodiments, the —O— end of a linker is connected to an APOC3 oligonucleotide. In some embodiments, the —O— end of a linker is connected to the 5′-end oligonucleotide (—O— being the oxygen in the 5′-OH).

In some embodiments, a linker, e.g., L^{M}, comprises a PO (phosphodiester linkage), a PS (phosphorothioate linkage) or PS2 (phosphorodithioate linkage). A non-limiting example including a PS linker is shown below. In some embodiments, a linker is —O—P(O)(OH)—O— [phosphodiester], —O—P(O)(SH)—O— [phosphorothioate] or —O—P(S)(SH)—O— [phosphorodithioate]. In some embodiments, a linker comprises a C₆ amino moiety (—NH—(CH₂)₆—), which is illustrated below. In some embodiments, a linker comprises a C₆ amino bound to a PO, a PS, or PS2. In some embodiments, a linker is a C₆ amino bound to a PO, a PS, or PS2. In some embodiments, a linker, e.g., L^{LD} or L, is —C(O)—NH—(CH₂)₆—P(O)(OH)—. In some embodiments, a linker, e.g., L^{LD} or L, is —C(O)—NH—(CH₂)₆—P(O)(OH)—, wherein —C(O)— is connected to a lipid moiety and —P(O)(OH)— is connected to an APOC3 oligonucleotide moiety. In some embodiments, a linker, e.g., L^{LD} or L, is —C(O)—NH—(CH₂)₆—P(O)(OH)—, wherein —C(O)— is connected to a lipid moiety and —P(O)(OH)— is connected to the 5′-O— of an APOC3 oligonucleotide moiety. In some embodiments, a linker, e.g., L^{LD} or L, is —C(O)—NH—(CH₂)₆—P(O)(OH)—, wherein —C(O)— is connected to a lipid moiety and —P(O)(OH)— is connected to the 3′-O— of an APOC3 oligonucleotide moiety. In some embodiments, a linker, e.g., L^{LD} or L, is —C(O)—NH—(CH₂)₆—P(O)(SH)—. In some embodiments, a linker, e.g., L^{LD} or L, is —C(O)—NH—(CH₂)₆—P(O)(SH)—, wherein —C(O)— is connected to a lipid moiety and —P(O)(SH)— is connected to an APOC3 oligonucleotide moiety. In some embodiments, a linker, e.g., L^{LD} or L, is —C(O)—NH—(CH₂)₆—P(O)(SH)—, wherein —C(O)— is connected to a lipid moiety and —P(O)(SH)— is connected to the 5′-O— of an APOC3 oligonucleotide moiety. In some embodiments, a linker, e.g., L^{LD} or L, is —C(O)—NH—(CH₂)₆—P(O)(SH)—, wherein —C(O)— is connected to a lipid moiety and —P(O)(SH)— is connected to the 3′-O— of an APOC3 oligonucleotide moiety. In some embodiments, a linker, e.g., L^{LD} or L, is —C(O)—NH—(CH₂)₆—P(S)(SH)—. In some embodiments, a linker, e.g., L^{LD} or L, is —C(O)—NH—(CH₂)₆—P(S)(SH)—, wherein —C(O)— is connected to a lipid moiety and —P(S)(SH)— is connected to an APOC3 oligonucleotide moiety. In some embodiments, a linker, e.g., L^{LD} or L, is —C(O)—NH—(CH₂)₆—P(S)(SH)—, wherein —C(O)— is connected to a lipid moiety and —P(S)(SH)— is connected to the 5′-O— of an APOC3 oligonucleotide moiety. In some embodiments, a linker, e.g., L^{LD} or L, is —C(O)—NH—(CH₂)₆—P(S)(SH)—, wherein —C(O)— is connected to a lipid moiety and —P(S)(SH)— is connected to the 3′-O— of an APOC3 oligonucleotide moiety. As appreciated by a person having ordinary skill in the art, at certain pH —P(O)(OH)—, —P(O)(SH)—, —P(S)(SH)— may exist as —P(O)(O⁻)—, —P(O)(S⁻)—, —P(S)(S⁻)—, respectively. In some embodiments, a lipid moiety is R^{LD}.

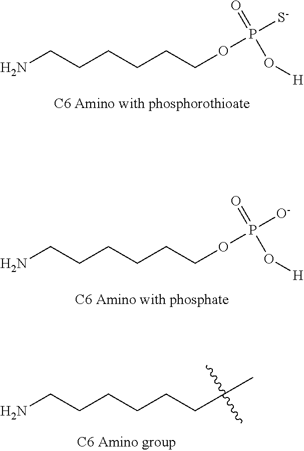 [SMILES: NCCCCCCOP(=O)(O)[S-] ; NCCCCCCOP(=O)([O-])O ; *CCCCCCN]

Various chemistry and linkers can be used for conjugation in accordance with the present disclosure. For example, in some embodiment, a lipid is incorporated using chemistry described below, or similar processes:

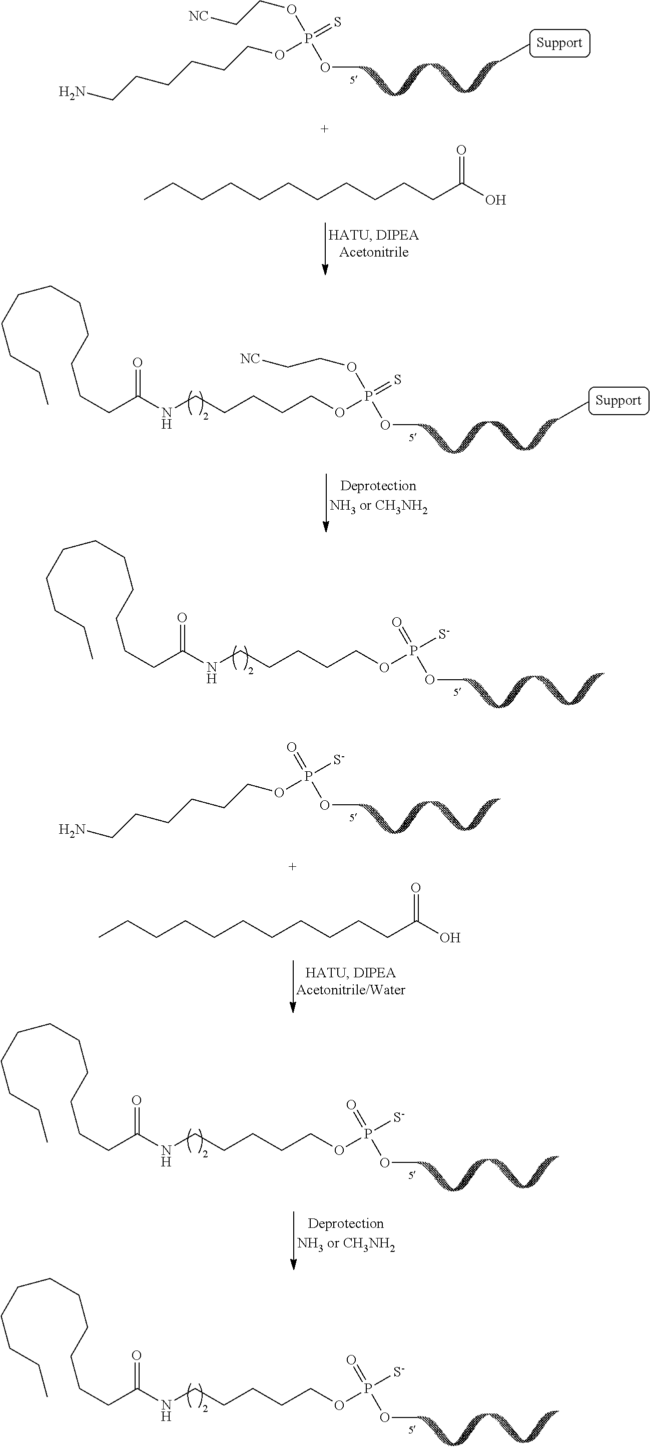

In some embodiments, a lipid is incorporated into an APOC3 oligonucleotide directly through a nucleobase, for example:

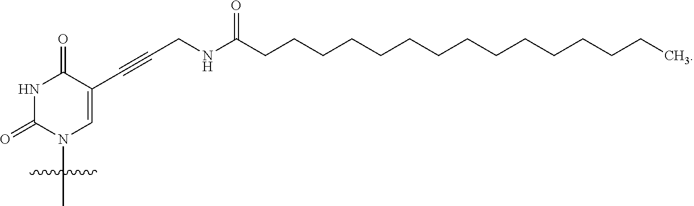 [SMILES: *n1cc(C#CCNC(=O)CCCCCCCCCCCCCCC)c(=O)[nH]c1=O]

In some embodiments, a provided oligonucleotide comprises -L^{M}-R^{LD} directly bonded to a nucleobase. In some embodiments, a provided oligonucleotide comprises

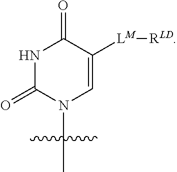

In some embodiments, a linker (L^{M}) is

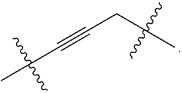 [SMILES: *C#CC*]

In some embodiments, a linker (L^{M}) is

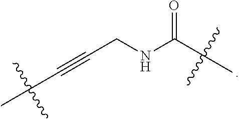 [SMILES: *C#CCNC(*)=O]

In some embodiments, a lipid moiety, R^{LD}, is

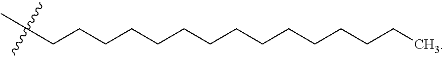 [SMILES: *CCCCCCCCCCCCCCC]

In some embodiments, a provided oligonucleotide comprises

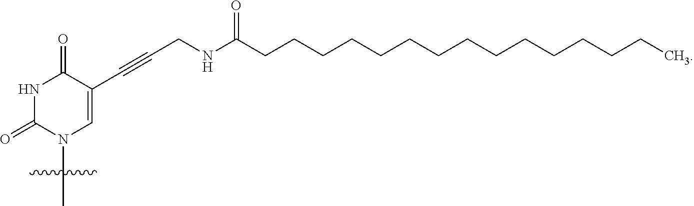 [SMILES: *n1cc(C#CCNC(=O)CCCCCCCCCCCCCCC)c(=O)[nH]c1=O]

In some embodiments, a provided oligonucleotide comprises a carbohydrate moiety connected to the oligonucleotide moiety, option through a linker, at a nucleobase. In some embodiments, the nucleobase is T. In some embodiments, the nucleobase is protected T. In some embodiments, the nucleobase is optionally substituted T. In some embodiments, the connection is at the 5-carbon of a T or an optionally substituted T. In some embodiments, a provided oligonucleotide comprises one or more -L^{M}-(R^{LD})a wherein each variable is independently as described in the present disclosure. In some embodiments, a provided oligonucleotide comprises one or more -L^{M}-(R^{LD})a, which is bonded to a nucleobase, wherein each variable is independently as described in the present disclosure. In some embodiments, a provided oligonucleotide comprises one or more

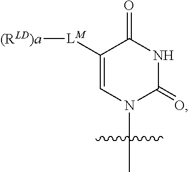

wherein each variable is independently as described in the present disclosure. In some embodiments, a provided oligonucleotide comprises one or more

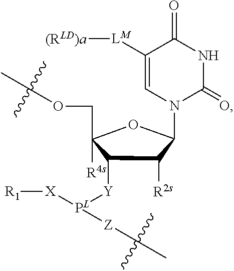

wherein each variable is independently as described in the present disclosure. In some embodiments, a provided oligonucleotide comprises one or more

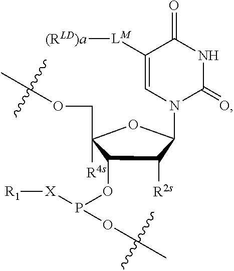

wherein each variable is independently as described in the present disclosure. In some embodiments, a provided oligonucleotide comprises one or more

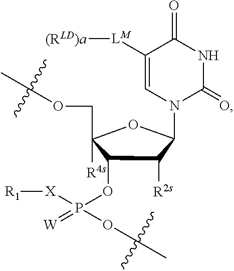

wherein each variable is independently as described in the present disclosure.
In some embodiments, a provided oligonucleotide comprises one or more

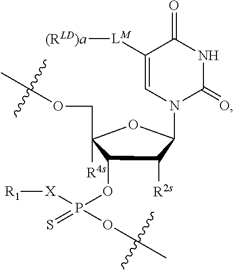

wherein each variable is independently as described in the present disclosure. In some embodiments, a provided oligonucleotide comprises one or more

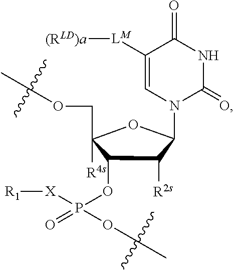

wherein each variable is independently as described in the present disclosure. In some embodiments, a provided oligonucleotide comprises one or more

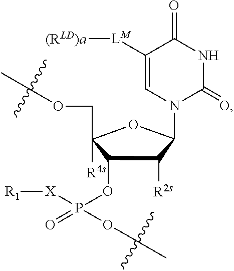

wherein X is O or S, R¹ is H, and each other variable is independently as described in the present disclosure. In some embodiments, R^{2s} and R^{4s} are hydrogen. In some embodiments, a provided oligonucleotide comprises one or more

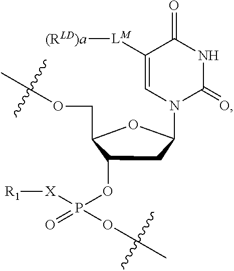

wherein X is O or S, R¹ is H, and each other variable is independently as described in the present disclosure.

In some embodiments, a is 1. In some embodiments, a provided oligonucleotide comprises one or more -L^{M}-R^{CD}, which is bonded to a nucleobase, wherein each variable is independently as described in the present disclosure. In some embodiments, the nucleobase is T. In some embodiments, the nucleobase is protected T. In some embodiments, the nucleobase is optionally substituted T. In some embodiments, the connection is at the 5-carbon of a T or an optionally substituted T. In some embodiments, a provided oligonucleotide comprises one or more

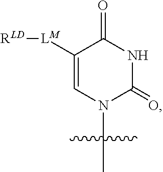

wherein each variable is independently as described in the present disclosure. In some embodiments, a provided oligonucleotide comprises one or more

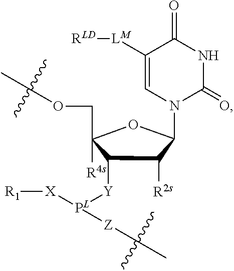

wherein each variable is independently as described in the present disclosure. In some embodiments, a provided oligonucleotide comprises one or more

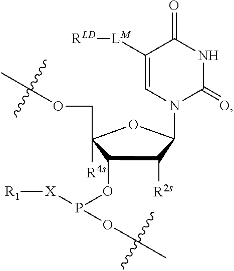

wherein each variable is independently as described in the present disclosure. In some embodiments, a provided oligonucleotide comprises one or more

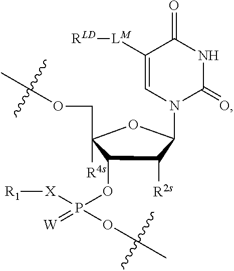

wherein each variable is independently as described in the present disclosure. In some embodiments, a provided oligonucleotide comprises one or more

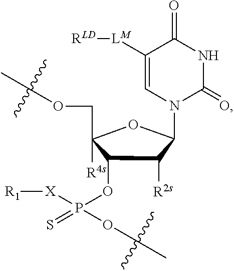

wherein each variable is independently as described in the present disclosure. In some embodiments, a provided oligonucleotide comprises one or more

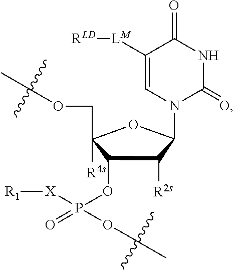

wherein each variable is independently as described in the present disclosure. In some embodiments, a provided oligonucleotide comprises one or more

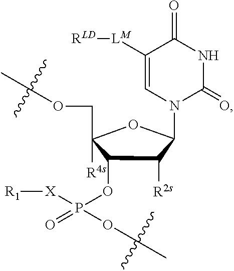

wherein X is O or S, R¹ is H, and each other variable is independently as described in the present disclosure. In some embodiments, R^{2s} and R^{4s} are hydrogen. In some embodiments, a provided oligonucleotide comprises one or more

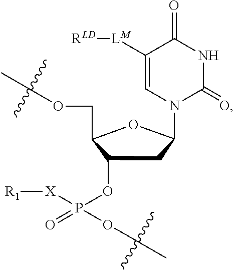

wherein X is O or S, R¹ is H, and each other variable is independently as described in the present disclosure.

In some embodiments, the present disclosure provides a composition comprising an APOC3 oligonucleotide comprising a lipid moiety comprising or being a C₁₀-C₄₀ linear, saturated or partially unsaturated, aliphatic chain. In some embodiments, the present disclosure provides a composition comprising an APOC3 oligonucleotide comprising a lipid moiety comprising or being a C₁₀-C₄₀ linear, saturated or partially unsaturated, aliphatic chain, optionally substituted with one or more C₁₋₄ aliphatic group.

In some embodiments, a composition comprises an APOC3 oligonucleotide comprising a lipid moiety formed through conjugation of a compound selected from: lauric acid, myristic acid, palmitic acid, stearic acid, oleic acid, linoleic acid, alpha-linolenic acid, gamma-linolenic acid, docosahexaenoic acid (cis-DHA), turbinaric acid, arachidonic acid, and dilinoleyl alcohol

In some embodiments, a linker is a moiety that connects two parts of a composition; as a non-limiting example, a linker physically connects an APOC3 oligonucleotide to a lipid. Non-limiting examples of suitable linkers include: an uncharged linker; a charged linker; a linker comprising an alkyl; a linker comprising a phosphate; a branched linker; an unbranched linker; a linker comprising at least one cleavage group; a linker comprising at least one redox cleavage group; a linker comprising at least one phosphate-based cleavage group; a linker comprising at least one acid-cleavage group; a linker comprising at least one ester-based cleavage group; a linker comprising at least one peptide-based cleavage group. In some embodiments, a linker is an uncharged linker or a charged linker. In some embodiments, a linker comprises an alkyl.

In some embodiments, a linker comprises a phosphate. In various embodiments, a phosphate can also be modified by replacement of a bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either linking oxygen or at both the linking oxygens. In some embodiments, the bridging oxygen is the 3′-oxygen of a nucleoside, replacement with carbon is done. In some embodiments, the bridging oxygen is the 5′-oxygen of a nucleoside, replacement with nitrogen is done. In various embodiments, the linker comprising a phosphate comprises any one or more of: a phosphorodithioate, phosphoramidate, boranophosphonoate, or a compound of formula (I):

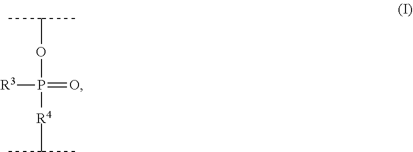

where R³ is selected from OH, SH, NH₂, BH₃, CH₃, C₁₋₆ alkyl, C₆₋₁₀ aryl, C₁₋₆ alkoxy and C₆₋₁₀ aryl-oxy, wherein C₁₋₆ alkyl and C₆₋₁₀ aryl are unsubstituted or optionally independently substituted with 1 to 3 groups independently selected from halo, hydroxyl and NH₂; and R⁴ is selected from O, S, NH, or CH₂.

In some embodiments, a linker comprises a direct bond or an atom such as oxygen or sulfur, a unit such as NR′, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R₁)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, a linker is a branched linker. In some embodiments, a branchpoint of the branched linker may be at least trivalent, but may be a tetravalent, pentavalent or hexavalent atom, or a group presenting such multiple valencies. In some embodiments, a branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

In one embodiment, a linker comprises at least one cleavable linking group. As a non-limiting example, a cleavable linking group can be sufficiently stable outside the cell, but which upon entry into a target cell is cleaved to release the two parts the linker is holding together. As a non-limiting example, a cleavable linkage group, such as a disulfide bond can be susceptible to pH. As a non-limiting example, a linker can include a cleavable linking group that is capable of being cleaved by an enzyme. As a non-limiting example, a linker can contain a peptide bond, which can be used when targeting cell types rich in peptidases, such as liver cells and synoviocytes. As a non-limiting example, suitability of a candidate cleavable linking group can be evaluated by testing the ability of a degradative agent (or condition) to cleave the candidate linking group. In some embodiments, a linker comprises a redox cleavable linking group, a phosphate-based cleavable linking groups, which are cleavable by agents that degrade or hydrolyze the phosphate group, a linker comprises an acid cleavable linking group, an ester-based linking group, and/or a peptide-based cleaving group.

Any linker reported in the art can be used, including, as non-limiting examples, those described in: U.S. Pat. App. No. 20150265708.

In some embodiments, a lipid is conjugated to an APOC3 oligonucleotide using any method known in the art in accordance with the present disclosure.

### Targeting Moieties

In some embodiments, a provided oligonucleotide or oligonucleotide composition further comprises a targeting component or moiety. A targeting moiety can be either conjugated or not conjugated to an APOC3 oligonucleotide moiety. In some embodiments, a targeting moiety is a lipid. In some embodiments, a targeting moiety is a carbohydrate or a bicyclic ketal. In some embodiments, a targeting moiety is —R^{LD} as described in the present disclosure. In some embodiments, a targeting moiety is —R^{CD} as described in the present disclosure.

Targeting moieties can be incorporated into provided technologies through many types of methods in accordance with the present disclosure, for example, those described for lipids and carbohydrates. In some embodiments, targeting moieties are physically mixed with provided oligonucleotides to form provided compositions. In some embodiments, a targeting moiety is conjugated to an APOC3 oligonucleotide. In some embodiments, a targeting moiety is not conjugated to an APOC3 oligonucleotide.

In some embodiments, provided compositions comprise two or more targeting moieties. In some embodiments, provided oligonucleotides comprise two or more conjugated targeting moieties. In some embodiments, the two or more conjugated targeting moieties are the same. In some embodiments, the two or more conjugated targeting moieties are different. In some embodiments, provided oligonucleotides comprise no more than one targeting moiety. In some embodiments, oligonucleotides of a provided composition comprise different types of conjugated targeting moieties. In some embodiments, oligonucleotides of a provided composition comprise the same type of targeting moieties.

Targeting moieties can be conjugated to oligonucleotides optionally through linkers, for example, as described for lipids and carbohydrates. Various types of linkers in the art can be utilized in accordance of the present disclosure. In some embodiments, a linker comprises a phosphate group, which can, for example, be used for conjugating targeting moieties through chemistry similar to those employed in oligonucleotide synthesis. In some embodiments, a linker comprises an amide, ester, or ether group. In some embodiments, a linker has the structure of -L-. Targeting moieties can be conjugated through either the same or different linkers compared to lipids.

Targeting moieties, optionally through linkers, can be conjugated to oligonucleotides at various suitable locations. In some embodiments, targeting moieties are conjugated through the 5′—OH group. In some embodiments, targeting moieties are conjugated through the 3′—OH group. In some embodiments, targeting moieties are conjugated through one or more sugar moieties. In some embodiments, targeting moieties are conjugated through one or more bases. In some embodiments, targeting moieties are incorporated through one or more internucleotidic linkages. In some embodiments, an APOC3 oligonucleotide may contain multiple conjugated targeting moieties which are independently conjugated through its 5′-OH, 3′-OH, sugar moieties, base moieties and/or internucleotidic linkages. Targeting moieties and lipids can be conjugated either at the same, neighboring and/or separated locations. In some embodiments, a targeting moiety is conjugated at one end of an APOC3 oligonucleotide, and a lipid is conjugated at the other end.

In some embodiments, a targeting moiety interacts with a protein on the surface of targeted cells. In some embodiments, such interaction facilitates internalization into targeted cells. In some embodiments, a targeting moiety comprises a sugar moiety. In some embodiments, a targeting moiety comprises a polypeptide moiety. In some embodiments, a targeting moiety comprises an antibody. In some embodiments, a targeting moiety is an antibody. In some embodiments, a targeting moiety comprises an inhibitor. In some embodiments, a targeting moiety is a moiety from a small molecule inhibitor. In some embodiments, an inhibitor is an inhibitor of a protein on the surface of targeted cells. In some embodiments, an inhibitor is a carbonic anhydrase inhibitor. In some embodiments, an inhibitor is a carbonic anhydrase inhibitor expressed on the surface of target cells. In some embodiments, a carbonic anhydrase is I, II, III, IV, V, VI, VII, VIII, IX, X, XI, XII, XIII, XIV, XV or XVI. In some embodiments, a carbonic anhydrase is membrane bound. In some embodiments, a carbonic anhydrase is IV, IX, XII or XIV. In some embodiments, an inhibitor is for IV, IX, XII and/or XIV. In some embodiments, an inhibitor is a carbonic anhydrase III inhibitor. In some embodiments, an inhibitor is a carbonic anhydrase IV inhibitor. In some embodiments, an inhibitor is a carbonic anhydrase IX inhibitor. In some embodiments, an inhibitor is a carbonic anhydrase XII inhibitor. In some embodiments, an inhibitor is a carbonic anhydrase XIV inhibitor. In some embodiments, an inhibitor comprises or is a sulfonamide (e.g., those described in Supuran, Conn. Nature Rev Drug Discover 2008, 7, 168-181, which sulfonamides are incorporated herein by reference). In some embodiments, an inhibitor is a sulfonamide. In some embodiments, targeted cells are muscle cells.

In some embodiments, a targeting moiety is R^{TD}, wherein R^{TD} is R^{LD} or R^{CD} as described in the present disclosure.

In some embodiments, a targeting moiety is R^{LD} as defined and described in the present disclosure. In some embodiments, the present disclosure provides oligonucleotides comprising R^{LD}. In some embodiments, the present disclosure provides oligonucleotide compositions comprising oligonucleotides comprising R^{LD}. In some embodiments, the present disclosure provides oligonucleotide compositions comprising a first plurality of oligonucleotides comprising R^{LD}. In some embodiments, the present disclosure provides chirally controlled oligonucleotide compositions of oligonucleotides comprising R^{LD}.

In some embodiments, a targeting moiety is R^{CD} as defined and described in the present disclosure. In some embodiments, the present disclosure provides oligonucleotides comprising R^{CD}. In some embodiments, the present disclosure provides oligonucleotide compositions comprising oligonucleotides comprising R^{CD}. In some embodiments, the present disclosure provides oligonucleotide compositions comprising a first plurality of oligonucleotides comprising R^{CD}. In some embodiments, the present disclosure provides chirally controlled oligonucleotide compositions of oligonucleotides comprising R^{CD}.

In some embodiments, R^{TD} comprises or is

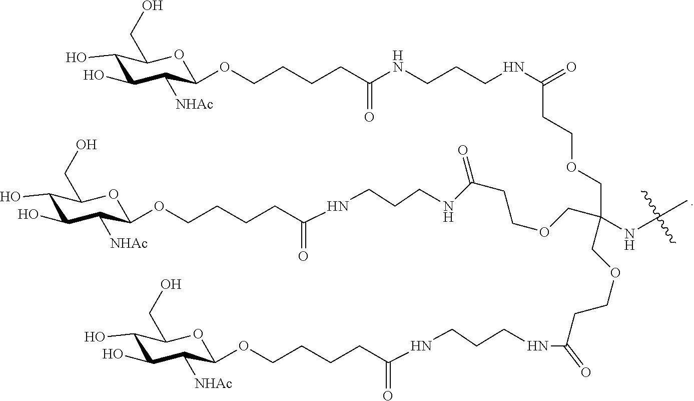

In some embodiments, R^{TD}comprises or is

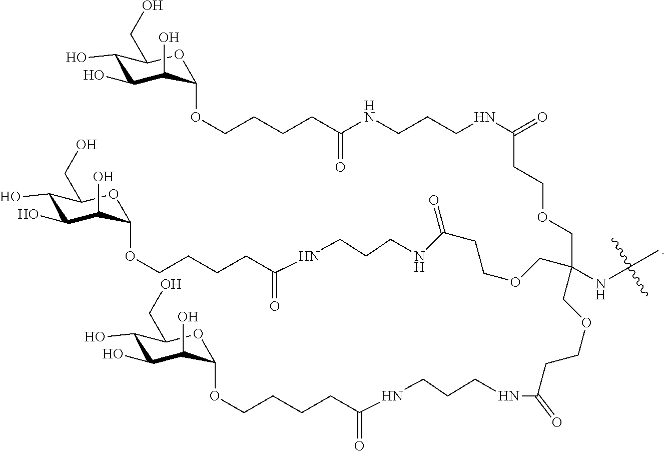

In some embodiments, R^{TD} comprises or is

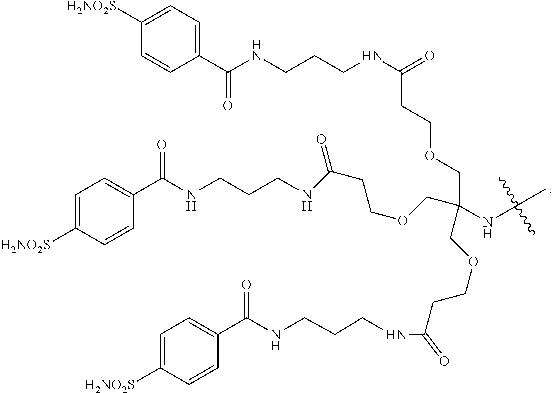 [SMILES: *NC(COCCC(=O)NCCCNC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, R^{ID} comprises or is

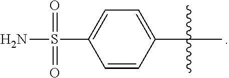 [SMILES: *c1ccc(S(N)(=O)=O)cc1]

In some embodiments, R^{TD} comprises or is

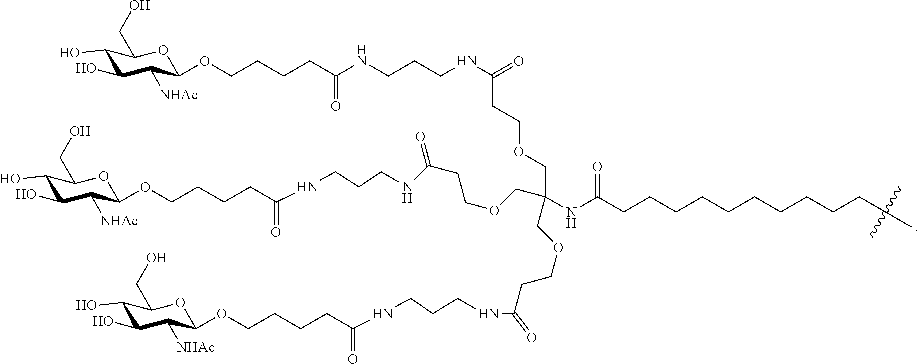

In some embodiments, R^{TD} comprises or is

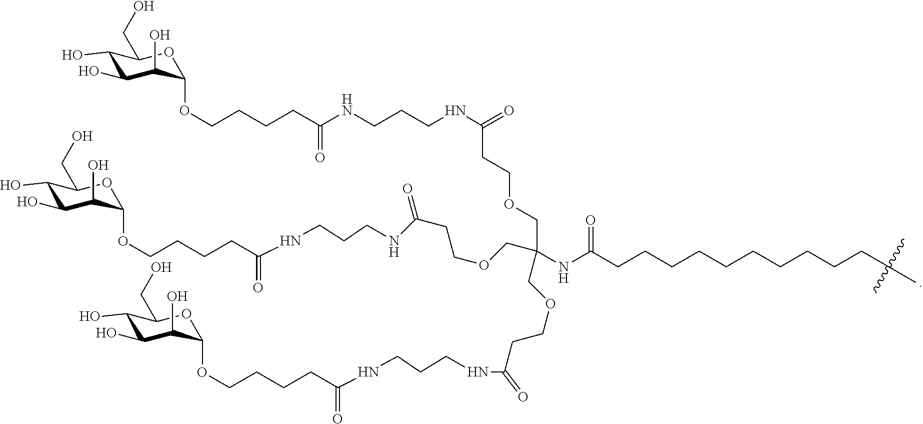

In some embodiments, R^{TD} comprises or is

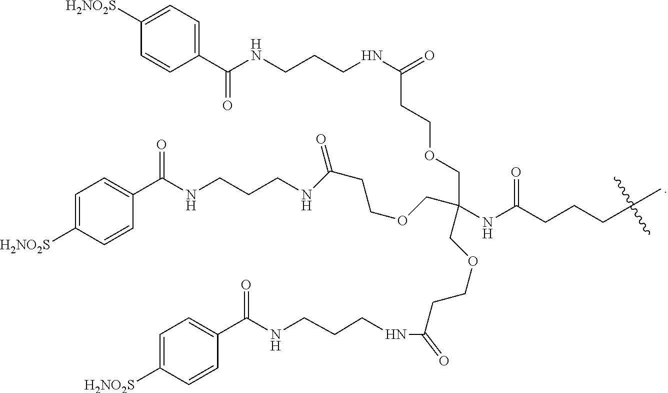 [SMILES: *CCCC(=O)NC(COCCC(=O)NCCCNC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, R^{TD} comprises or is

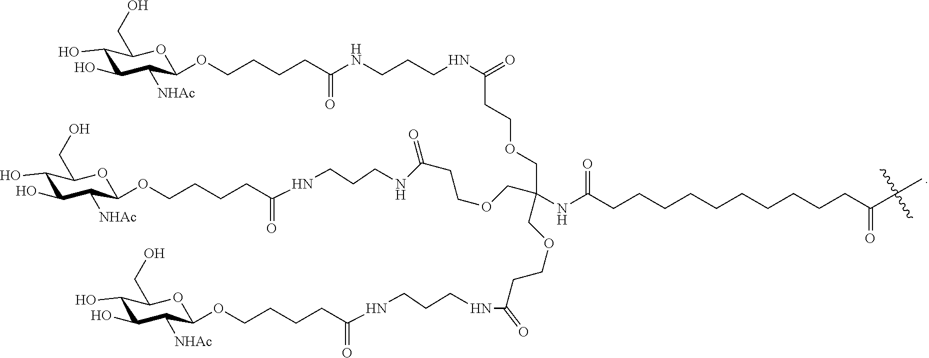

In some embodiments, R^{TD} comprises or is

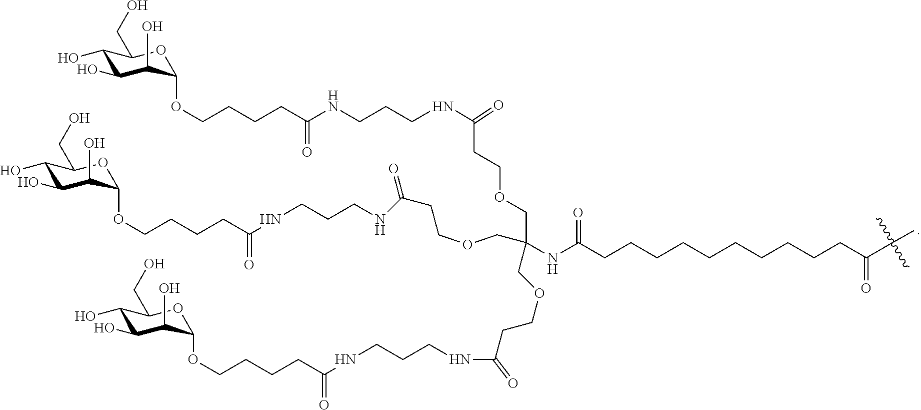

In some embodiments, R^{TD} comprises or is

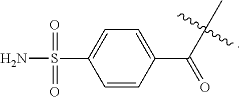 [SMILES: *C(=O)c1ccc(S(N)(=O)=O)cc1]

In some embodiments, R^{TD} comprises or

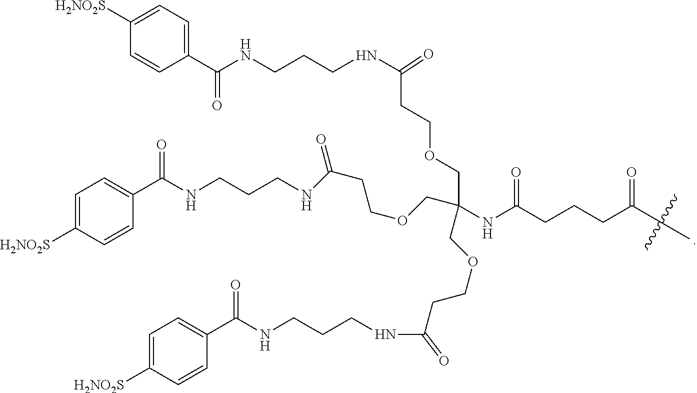 [SMILES: *C(=O)CCCC(=O)NC(COCCC(=O)NCCCNC(=O)c1ccc(S(N)(=O)=O)cc1)(COCCC(=O)NCCCNC(=O)c1ccc(S(N)(=O)=O)cc1)COCCC(=O)NCCCNC(=O)c1ccc(S(N)(=O)=O)cc1]

In some embodiments, R^{TD} comprises or is

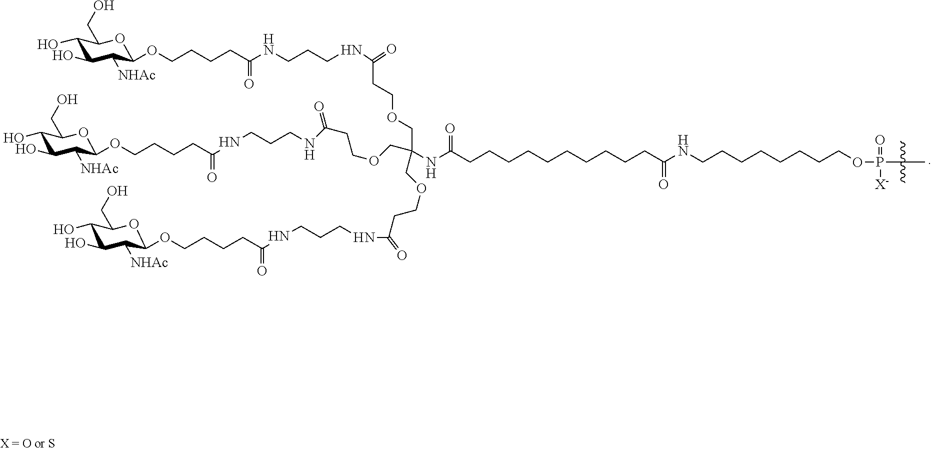

In some embodiments, R^{TD} comprises or is

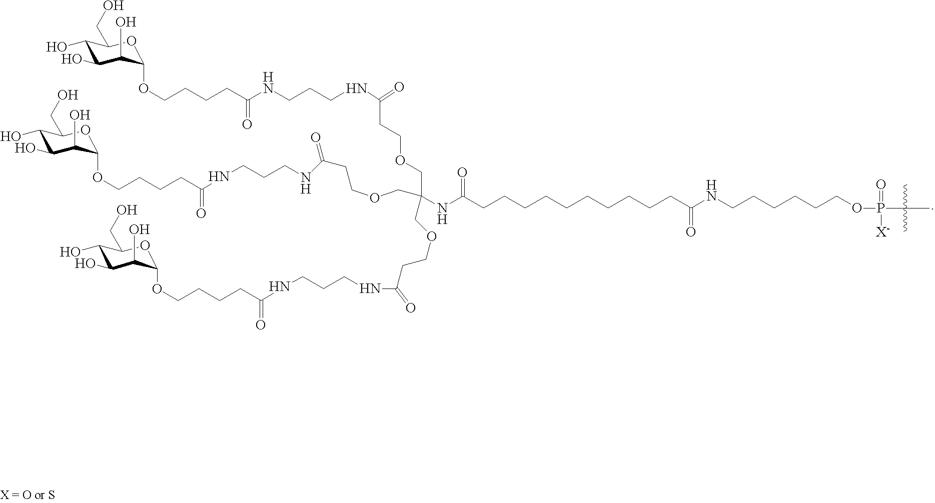

In some embodiments, R^{TD} comprises or is

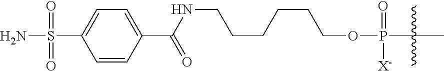

X═O or S. In some embodiments, R^{TD} comprises or is

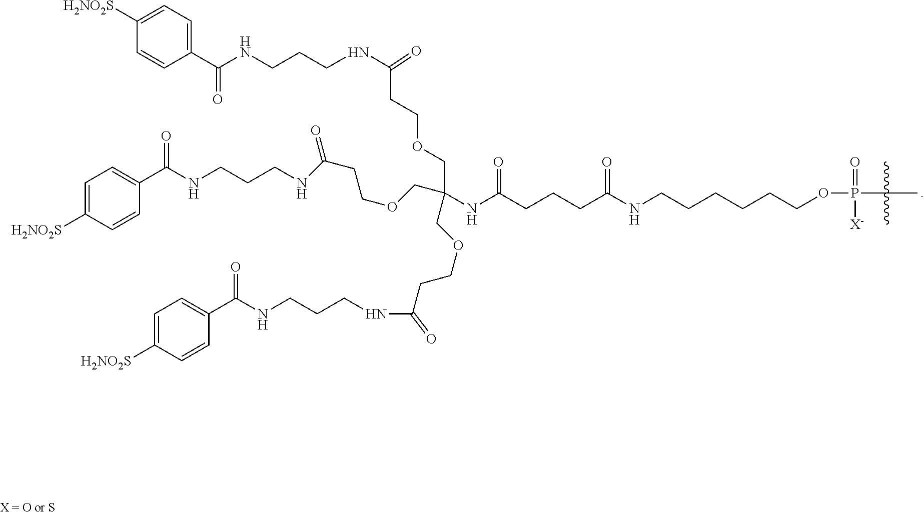

In some embodiments, R^{TD} is a targeting moiety that comprises or is a lipid moiety. In some embodiments, X is O. In some embodiments, X is S.

In some embodiments, the present disclosure provides technologies (e.g., reagents, methods, etc.) for conjugating various moieties to oligonucleotide moieties. In some embodiments, the present disclosure provides technologies for conjugating targeting moiety to oligonucleotide moieties. In some embodiments, the present disclosure provides acids comprising targeting moieties for conjugation, e.g., R^{LD}—COOH. In some embodiments, the present disclosure provides linkers for conjugation, e.g., L^{M}. A person having ordinary skill in the art understands that many known and widely practiced technologies can be utilized for conjugation with oligonucleotide moieties in accordance with the present disclosure. In some embodiments, a provided acid is

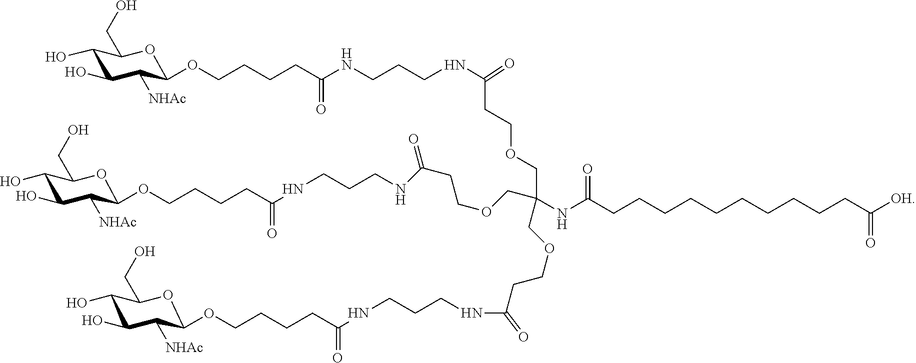

In some embodiments, a provided acid is

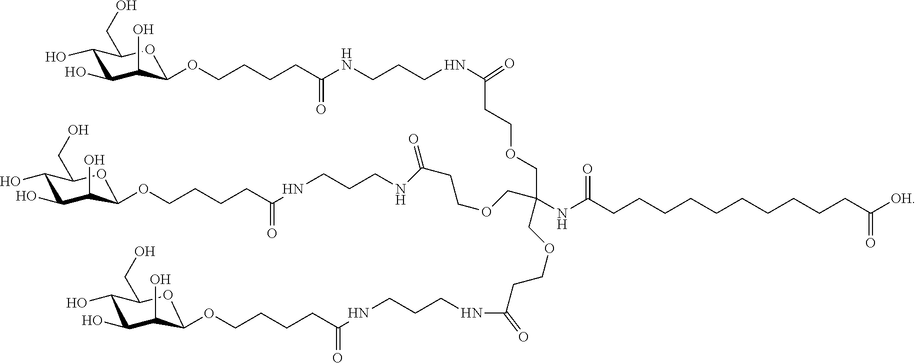

In some embodiments, a provided acid is

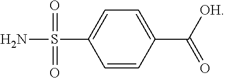 [SMILES: NS(=O)(=O)c1ccc(C(=O)O)cc1]

In some embodiments, a provided acid is

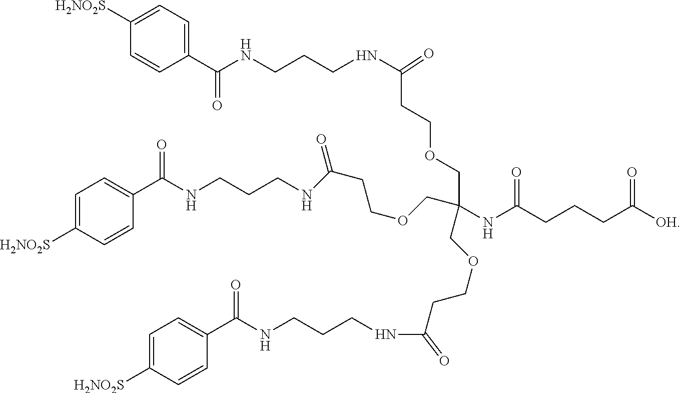 [SMILES: NS(=O)(=O)c1ccc(C(=O)NCCCNC(=O)CCOCC(COCCC(=O)NCCCNC(=O)c2ccc(S(N)(=O)=O)cc2)(COCCC(=O)NCCCNC(=O)c2ccc(S(N)(=O)=O)cc2)NC(=O)CCCC(=O)O)cc1]

In some embodiments, a provided acid is a fatty acid, which can provide a lipid moiety as a targeting moiety. In some embodiments, the present disclosure provides methods and reagents for preparing such acids.

In some embodiments, an APOC3 oligonucleotide, an APOC3 oligonucleotide capable of directing a decrease in the expression and/or level of a target gene or its gene product can comprise any lipid described herein or known in the art.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any lipid described herein or known in the art.

In some embodiments, a provided oligonucleotide comprises a lipid moiety. In some embodiments, a lipid moiety is incorporated by conjugation with a lipid. In some embodiments, a lipid is a fatty acid. In some embodiments, an APOC3 oligonucleotide is conjugated to a fatty acid. In some embodiments, a provided single-stranded RNAi agent comprises a lipid. In some embodiments, a provided single-stranded RNAi agent comprises a lipid conjugated to a nucleotide in the seed region. In some embodiments, a provided single-stranded RNAi agent comprises a lipid conjugated to a nucleotide in the post-seed region. In some embodiments, a provided single-stranded RNAi agent comprises a lipid conjugated at the 1^{st}, 2^{nd}, 3 ^{rd}, 4 ^{th}, 5 ^{th}, 6 ^{th}, 7 ^{th}, 8 ^{th}, 9 ^{th}, 10 ^{th}, 11 ^{th}, 12 ^{th}, 13 ^{th}, 14 ^{th}, 15 ^{th}, 16 ^{th}, 17 ^{th}, 18 ^{th}, 19 ^{th}, 20 ^{th}, 21^{st}, 22^{nd}, 23^{rd}, 24^{th}, or 25^{th} nucleotide (counting from the 5′-end). In some embodiments, a provided single-stranded RNAi agent comprises a lipid conjugated at the 9^{th} or 11^{th} nucleotide (counting from the 5′-end). In some embodiments, an APOC3 oligonucleotide is conjugated at the base to a fatty acid. In some embodiments, a provided single-stranded RNAi agent comprises a lipid. In some embodiments, a provided single-stranded RNAi agent comprises a lipid conjugated at the base at the 9^{th} or 11^{th} nucleotide (counting from the 5′-end).

In some embodiments, a fatty acid comprises 10, 11, 12, 13, 14, 15, 16, 17, 18, 19, 20, 21, 22, 23, 24, 25, 26, 27, 28, 29, 30 or more carbon atoms. In some embodiments, a fatty acid comprises 10 or more carbon atoms. In some embodiments, a fatty acid comprises 11 or more carbon atoms. In some embodiments, a fatty acid comprises 12 or more carbon atoms. In some embodiments, a fatty acid comprises 13 or more carbon atoms. In some embodiments, a fatty acid comprises 14 or more carbon atoms. In some embodiments, a fatty acid comprises 15 or more carbon atoms. In some embodiments, a fatty acid comprises 16 or more carbon atoms. In some embodiments, a fatty acid comprises 17 or more carbon atoms. In some embodiments, a fatty acid comprises 18 or more carbon atoms. In some embodiments, a fatty acid comprises 19 or more carbon atoms. In some embodiments, a fatty acid comprises 20 or more carbon atoms. In some embodiments, a fatty acid comprises 30 or more carbon atoms.

In some embodiments, a lipid is palmitic acid. In some embodiments, a lipid is stearic acid or turbinaric acid. In some embodiments, a lipid is stearic acid acid. In some embodiments, a lipid is turbinaric acid.

In some embodiments, a lipid comprises an optionally substituted, C₁₀-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂, —Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein.

In some embodiments, a lipid comprises an optionally substituted C₁₀-C₆₀ saturated or partially unsaturated, aliphatic chain.

In some embodiments, a lipid comprises an optionally substituted C₁₀-C₆₀ linear, saturated or partially unsaturated, aliphatic chain.

In some embodiments, a lipid comprises a C₁₀-C₆₀ linear, saturated or partially unsaturated, aliphatic chain, optionally substituted with one or more C₁₋₄ aliphatic group.

In some embodiments, a lipid comprises an optionally substituted, C₁₀-C₆₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂, —Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein.

In some embodiments, a lipid comprises an optionally substituted C₁₀-C₆₀ saturated or partially unsaturated, aliphatic chain.

In some embodiments, a lipid comprises an optionally substituted C₁₀-C₆₀ linear, saturated or partially unsaturated, aliphatic chain.

In some embodiments, a lipid comprises a C₁₀-C₆₀ linear, saturated or partially unsaturated, aliphatic chain, optionally substituted with one or more C₁₋₄ aliphatic group.

In some embodiments, a lipid comprises an optionally substituted, C₁₀-C₄₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂, —Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—, wherein each variable is independently as defined and described herein.

In some embodiments, a lipid comprises an optionally substituted C₁₀-C₆₀ saturated or partially unsaturated, aliphatic chain.

In some embodiments, a lipid comprises an optionally substituted C₁₀-C₆₀ linear, saturated or partially unsaturated, aliphatic chain.

In some embodiments, a lipid comprises a C₁₀-C₆₀ linear, saturated or partially unsaturated, aliphatic chain, optionally substituted with one or more C₁₋₄ aliphatic group.

In some embodiments, a lipid comprises an unsubstituted C₁₀-C₈₀ linear, saturated or partially unsaturated, aliphatic chain.

In some embodiments, a lipid comprises no more than one optionally substituted C₁₀-C₆₀ linear, saturated or partially unsaturated, aliphatic chain.

In some embodiments, a lipid comprises two or more optionally substituted C₁₀-C₆₀ linear, saturated or partially unsaturated, aliphatic chain.

In some embodiments, a lipid comprises an unsubstituted C₁₀-C₆₀ linear, saturated or partially unsaturated, aliphatic chain.

In some embodiments, a lipid comprises no more than one optionally substituted C₁₀-C₆₀ linear, saturated or partially unsaturated, aliphatic chain.

In some embodiments, a lipid comprises two or more optionally substituted C₁₀-C₆₀ linear, saturated or partially unsaturated, aliphatic chain.

In some embodiments, a lipid comprises an unsubstituted C₁₀-C₄₀ linear, saturated or partially unsaturated, aliphatic chain.

In some embodiments, a lipid comprises no more than one optionally substituted C₁₀-C₆₀ linear, saturated or partially unsaturated, aliphatic chain.

In some embodiments, a lipid comprises two or more optionally substituted C₁₀-C₆₀ linear, saturated or partially unsaturated, aliphatic chain.

In some embodiments, a lipid comprises a C₁₀-C₄₀ linear, saturated or partially unsaturated, aliphatic chain.

In some embodiments, a lipid comprises a C₁₀-C₃₀ linear, saturated or partially unsaturated, aliphatic chain.

In some embodiments, a lipid comprises a C₁₀-C₂₀ linear, saturated or partially unsaturated, aliphatic chain.

In some embodiments, a lipid comprises a C₁₀-C₁₆ linear, saturated or partially unsaturated, aliphatic chain.

In some embodiments, a lipid comprises a C₁₂-C₁₆ linear, saturated or partially unsaturated, aliphatic chain.

In some embodiments, a lipid comprises a C₁₄-C₁₆ linear, saturated or partially unsaturated, aliphatic chain.

In some embodiments, a lipid comprises a C₁₀-C₄₀ linear, saturated or partially unsaturated, aliphatic chain, optionally substituted with one or more C₁₋₄ aliphatic group.

In some embodiments, a lipid is selected from the group consisting of: lauric acid, myristic acid, palmitic acid, stearic acid, oleic acid, linoleic acid, alpha-linolenic acid, gamma-linolenic acid, docosahexaenoic acid (DHA or cis-DHA), turbinaric acid and dilinoleyl.

In some embodiments, a lipid is not conjugated to the oligonucleotide.

In some embodiments, a lipid is conjugated to the oligonucleotide.

In some embodiments, a lipid is conjugated to the oligonucleotide with a linker. In some embodiments, a linker has the structure of -L-.

In some embodiments, a targeting moiety is conjugated to an APOC3 oligonucleotide. In some embodiments, a provided oligonucleotide comprises one or more targeting moieties. In some embodiments, a targeting moiety is conjugated via a linker.

In some embodiments, a provided oligonucleotide comprises one or more lipid moieties, and one or more targeting moieties.

In some embodiments, a provided single-stranded RNAi agent comprises a lipid. In some embodiments, a provided single-stranded RNAi agent comprises a lipid moiety, wherein the lipid is C₁₆ linear. In some embodiments, a provided single-stranded RNAi agent comprises a lipid, wherein the lipid is palmitic acid.

In some embodiments, a provided single-stranded RNAi agent comprises a lipid conjugated to a base. In some embodiments, a provided single-stranded RNAi agent comprises a lipid, wherein the lipid is C₁₆ linear conjugated to a base. In some embodiments, a provided single-stranded RNAi agent comprises a lipid, wherein the lipid is palmitic acid conjugated to a base.

In some embodiments, the present disclosure pertains to a chirally controlled oligonucleotide composition, wherein the composition further comprises a lipid. In some embodiments, a lipid is stearic acid or turbinaric acid. In some embodiments, a lipid is conjugated to the oligonucleotide.

In some embodiments, conjugation of a lipid to an APOC3 oligonucleotide improves at least one property of the oligonucleotide. In some embodiments, the property is increased activity (e.g., increased ability to mediate single-stranded RNA interference), or improved distribution to a tissue. In some embodiments, lipid conjugation improves activity. In some embodiments, lipid conjugation improves deliveries to one or more target tissues. In some embodiments, the tissue is muscle tissue. In some embodiments, the tissue is skeletal muscle, gastrocnemius, triceps, heart or diaphragm.

In some embodiments, a lipid comprises an optionally substituted, C10-C80 saturated or partially unsaturated aliphatic group, wherein one or more methylene units are optionally and independently replaced by an optionally substituted group selected from C1-C6 alkylene, C1-C6 alkenylene, a C1-C6 heteroaliphatic moiety, —C(R)₂—, —Cy-, —O—, —S—, —S—S—, —N(R)—, —C(O)—, —C(S)—, —C(NR)—, —C(O)N(R)—, —N(R)C(O)N(R), —N(R)C(O)—, —N(R)C(O)O—, —OC(O)N(R)—, —S(O)—, —S(O)2-, —S(O)2N(R)—, —N(R)S(O)₂—, —SC(O)—, —C(O)S—, —OC(O)—, and —C(O)O—, wherein each variable is independently as defined and described herein. In some embodiments, a lipid comprises an optionally substituted C10-C60 saturated or partially unsaturated, aliphatic chain. In some embodiments, a lipid comprises an optionally substituted C10-C60 linear, saturated or partially unsaturated, aliphatic chain. In some embodiments, a lipid comprises a C10-C60 linear, saturated or partially unsaturated, aliphatic chain, optionally substituted with one or more C1-4 aliphatic group. In some embodiments, a lipid comprises an optionally substituted, C10-C60 saturated or partially unsaturated aliphatic group, wherein one or more methylene units are optionally and independently replaced by an optionally substituted group selected from C1-C6 alkylene, C1-C6 alkenylene, a C1-C6 heteroaliphatic moiety, —C(R)₂—, —Cy-, —O—, —S—, —S—S—, —N(R)—, —C(O)—, —C(S)—, —C(NR)—, —C(O)N(R)—, —N(R)C(O)N(R), —N(R)C(O)—, —N(R)C(O)O—, —OC(O)N(R)—, —S(O)—, —S(O)₂—, —S(O)₂N(R)—, —N(R)S(O)₂—, —SC(O)—, —C(O)S—, —OC(O)—, and —C(O)O—, wherein each variable is independently as defined and described herein. In some embodiments, a lipid comprises an optionally substituted C10-C60 saturated or partially unsaturated, aliphatic chain. In some embodiments, a lipid comprises an optionally substituted C10-C60 linear, saturated or partially unsaturated, aliphatic chain. In some embodiments, a lipid comprises a C10-C60 linear, saturated or partially unsaturated, aliphatic chain, optionally substituted with one or more C1-4 aliphatic group. In some embodiments, a lipid comprises an optionally substituted, C10-C40 saturated or partially unsaturated aliphatic group, wherein one or more methylene units are optionally and independently replaced by an optionally substituted group selected from C1-C6 alkylene, C1-C6 alkenylene, a C1-C6 heteroaliphatic moiety, —C(R)₂—, —Cy-, —O—, —S—, —S—S—, —N(R)—, —C(O)—, —C(S)—, —C(NR)—, —C(O)N(R)—, —N(R)C(O)N(R), —N(R)C(O)—, —N(R)C(O)O—, —OC(O)N(R)—, —S(O)—, —S(O)₂—, —S(O)₂N(R)—, —N(R)S(O)₂—, —SC(O)—, —C(O)S—, —OC(O)—, and —C(O)O—, wherein each variable is independently as defined and described herein. In some embodiments, a lipid comprises an optionally substituted C10-C60 saturated or partially unsaturated, aliphatic chain. In some embodiments, a lipid comprises an optionally substituted C10-C60 linear, saturated or partially unsaturated, aliphatic chain. In some embodiments, a lipid comprises a C10-C60 linear, saturated or partially unsaturated, aliphatic chain, optionally substituted with one or more C1-4 aliphatic group. In some embodiments, a lipid comprises an unsubstituted C10-C80 linear, saturated or partially unsaturated, aliphatic chain. In some embodiments, a lipid comprises no more than one optionally substituted C10-C60 linear, saturated or partially unsaturated, aliphatic chain. In some embodiments, a lipid comprises two or more optionally substituted C10-C60 linear, saturated or partially unsaturated, aliphatic chain. In some embodiments, a lipid comprises an unsubstituted C10-C60 linear, saturated or partially unsaturated, aliphatic chain. In some embodiments, a lipid comprises no more than one optionally substituted C10-C60 linear, saturated or partially unsaturated, aliphatic chain. In some embodiments, a lipid comprises two or more optionally substituted C10-C60 linear, saturated or partially unsaturated, aliphatic chain. In some embodiments, a lipid comprises an unsubstituted C10-C40 linear, saturated or partially unsaturated, aliphatic chain. In some embodiments, a lipid comprises no more than one optionally substituted C10-C60 linear, saturated or partially unsaturated, aliphatic chain. In some embodiments, a lipid comprises two or more optionally substituted C10-C60 linear, saturated or partially unsaturated, aliphatic chain. In some embodiments, a lipid comprises a C10-C40 linear, saturated or partially unsaturated, aliphatic chain. In some embodiments, a lipid comprises a C10-C40 linear, saturated or partially unsaturated, aliphatic chain, optionally substituted with one or more C1-4 aliphatic group. In some embodiments, the lipid is selected from the group consisting of: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl. In some embodiments, the lipid is not conjugated to the oligonucleotide. In some embodiments, the lipid is conjugated to the oligonucleotide.

In some embodiments, conjugation of a lipid to an APOC3 oligonucleotide surprisingly improves at least one property of the oligonucleotide. In some embodiments, the property is increased activity (e.g., increased ability to mediate single-stranded RNA interference), or improved distribution to a tissue. In some embodiments, the tissue is muscle tissue. In some embodiments, the tissue is skeletal muscle, gastrocnemius, triceps, heart or diaphragm. In some embodiments, oligonucleotides comprising lipid moieties form, for example, micelles. In some embodiments, example improved properties are demonstrated, e.g., in one or more of the Figures.

In some embodiments, when assaying example oligonucleotides in mice, tested oligonucleotides are intravenous injected via tail vein in male C57BL/10ScSnDMDmdx mice (4-5 weeks old), at tested amounts, e.g., 10 mg/kg, 30 mg/kg, etc. In some embodiments, tissues are harvested at tested times, e.g., on Day, e.g., 2, 7 and/or 14, etc., after injection, in some embodiments, fresh-frozen in liquid nitrogen and stored in −80° C. until analysis.

Various assays can be used to assess oligonucleotide levels in accordance with the present disclosure. In some embodiments, hybrid-ELISA is used to quantify oligonucleotide levels in tissues using test article serial dilution as standard curve: for example, in an example procedure, maleic anhydride activated 96 well plate (Pierce 15110) was coated with 50 l of capture probe at 500 nM in 2.5% NaHCO₃ (Gibco, 25080-094) for 2 hours at 37° C. The plate was then washed 3 times with PBST (PBS+0.1% Tween-20), and blocked with 5% fat free milk-PBST at 37° C. for 1 hour. Test article oligonucleotide was serial diluted into matrix. This standard together with original samples were diluted with lysis buffer (4 M Guanidine; 0.33% N-Lauryl Sarcosine; 25 mM Sodium Citrate; 10 mM DTT) so that oligonucleotide amount in all samples is less than 100 ng/ml. 20 l of diluted samples were mixed with 180 l of 333 nM detection probe diluted in PBST, then denatured in PCR machine (65° C., 10 min, 95° C., 15 min, 4° C.). 50 l of denatured samples were distributed in blocked ELISA plate in triplicates, and incubated overnight at 4° C. After 3 washes of PBST, 1:2000 streptavidin-AP in PBST was added, 50 l per well and incubated at room temperature for 1 hour. After extensive wash with PBST, 100 l of AttoPhos (Promega S1000) was added, incubated at room temperature in dark for 10 min and read on plate reader (Molecular Device, M5) fluorescence channel. Ex435 nm, Em555 nm. Oligonucleotides in samples were calculated according to standard curve by 4-parameter regression.

As described and demonstrated in the present disclosure, in some embodiments, lipid conjugation improves delivery to a tissue. In some embodiments, lipid conjugation improves delivery to muscle. In some embodiments, lipid conjugation comprises conjugation with a fatty acid. In some embodiments, oligonucleotides are conjugated with turbinaric acid. In some embodiments, conjugation with turbinaric acid is particularly effective in improving oligonucleotide delivery to muscle.

In some embodiments, provided oligonucleotides are stable in both plasma and tissue homogenates.

In some embodiments, a provided single-stranded RNAi agent comprises a lipid conjugated at position 9 or 11 (counting from the 5′-end). In some embodiments, a provided single-stranded RNAi agent comprises a lipid, wherein the lipid is C₁₆ linear conjugated at position 9 or 11 (counting from the 5′-end). In some embodiments, a provided single-stranded RNAi agent comprises a lipid, wherein the lipid is palmitic acid conjugated at position 9 or 11 (counting from the 5′-end).

In some embodiments, a provided single-stranded RNAi agent comprises a lipid conjugated to a base at position 9 or 11 (counting from the 5′-end). In some embodiments, a provided single-stranded RNAi agent comprises a lipid, wherein the lipid is C₁₆ linear conjugated to a base at position 9 or 11 (counting from the 5′-end). In some embodiments, a provided single-stranded RNAi agent comprises a lipid, wherein the lipid is palmitic acid conjugated to a base at position 9 or 11 (counting from the 5′-end).

In some embodiments, a provided single-stranded RNAi agent comprises a lipid conjugated to a U base at position 9 or 11 (counting from the 5′-end). In some embodiments, a provided single-stranded RNAi agent comprises a lipid, wherein the lipid is C₁₆ linear conjugated to a U base at position 9 or 11 (counting from the 5′-end). In some embodiments, a provided single-stranded RNAi agent comprises a lipid, wherein the lipid is palmitic acid conjugated to a U base at position 9 or 11 (counting from the 5′-end).

In some embodiments, a provided single-stranded RNAi comprises a structure of ImU, or 5′-lipid-2′OMeU.

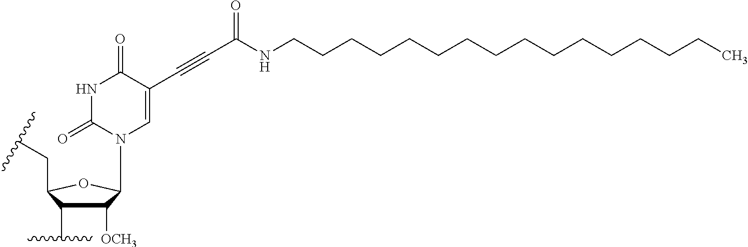

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any additional chemical moiety, including but not limited to, a lipid, described in any of U.S. Pat. Nos. 5,614,503; 5,780,009; 6,074,863; 6,258,581; 6,489,117; 6,677,445; 6,828,435; 6,846,921; 7,416,849; 7,494,982; 7,981,871; 8,106,022; 8,148,344; 8,318,508; 8,389,707; 8,450,467; 8,507,455; 8,703,731; 8,828,956; 8,901,046; 9,107,904; 9,352,048; 9,370,581; 9,370,582; 9,387,257; 9,388,415; 9,388,416; 9,393,316; and 9,404,112.

Optional Additional Chemical Moieties Conjugated to an APOC3 Oligonucleotide: A Carbohydrate Moiety or a Bicyclic Ketal, Including but not Limited to, a GalNAc Moiety

In some embodiments, provided oligonucleotides or oligonucleotide compositions comprise one or more carbohydrates or carbohydrate moieties or bicyclic ketal moieties. In some embodiments, a carbohydrate moiety is a carbohydrate. In some embodiments, a carbohydrate moiety is or comprises a carbohydrate which is conjugated directly or indirectly to an APOC3 oligonucleotide. In some embodiments, carbohydrate moieties facilitate targeted delivery of oligonucleotides to desired locations, e.g., cells, tissues, organs, etc. In some embodiments, provided carbohydrate moieties facilitate delivery to liver. As appreciated by a personal having ordinary skill in the art, various carbohydrate moieties are described in the literature and can be utilized in accordance with the present disclosure.

Carbohydrate moieties can be incorporated into oligonucleotides at various locations, for example, sugar units, internucleotidic linkage units, nucleobase units, etc., optionally through one or more bivalent or multivalent (which can be used to connect two or more carbohydrate moieties to oligonucleotides) linkers. In some embodiments, the present disclosure provides technologies for carbohydrate incorporation into oligonucleotides. In some embodiments, the present disclosure provides technologies for incorporating carbohydrate moieties, optionally through one or more linkers, at nucleobase units, as alternative and/or addition to incorporation at internucleotidic linkages and/or sugar units, thereby providing enormous flexibility and/or improved properties and/or activities. In some embodiments, a provided oligonucleotide comprises at least one carbohydrate moiety, optionally through a linker, incorporated into the oligonucleotide at a nucleobase unit.

In some embodiments, a linker is L^{M}, wherein L^{M} is a covalent bond, or a bivalent or multivalent, optionally substituted, linear or branched group selected from a C₁₋₁₀₀ aliphatic group and a C₁₋₁₀₀ heteroaliphatic group having 1-30 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy. In some embodiments, L^{M} is bivalent. In some embodiments, L^{M} is multivalent. In some embodiments, L^{M} is

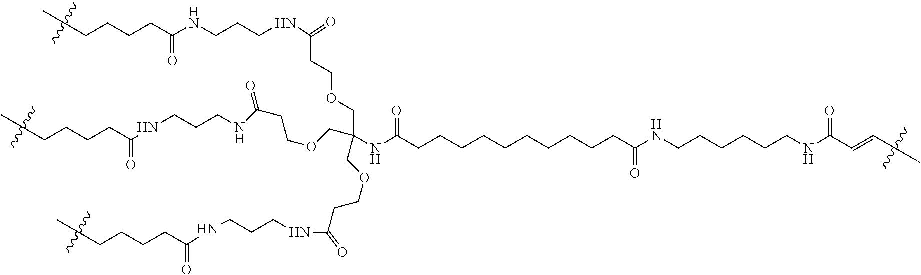

wherein L^{M} is directly bond to a nucleobase, for example, as in:

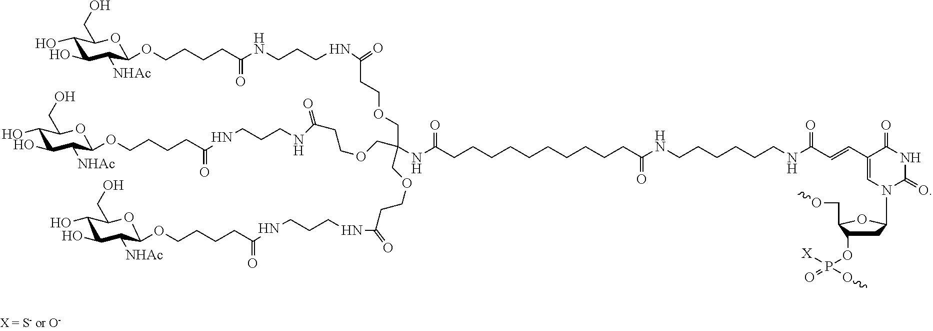

In some embodiments, L^{M} is

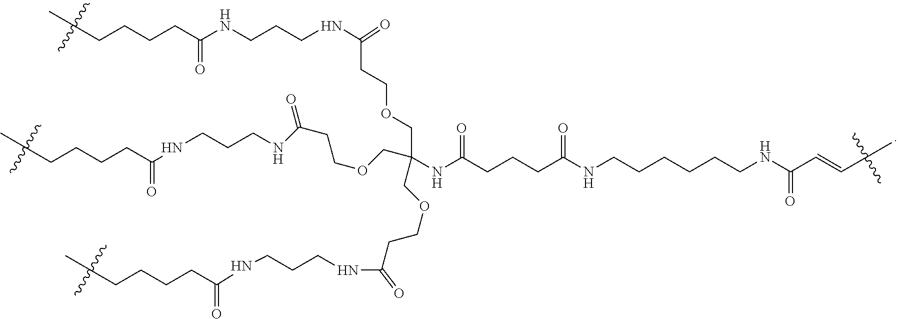 [SMILES: */C=C/C(=O)NCCCCCCNC(=O)CCCC(=O)NC(COCCC(=O)NCCCNC(=O)CCCC*)(COCCC(=O)NCCCNC(=O)CCCC*)COCCC(=O)NCCCNC(=O)CCCC*]

In some embodiments, L^{M} is

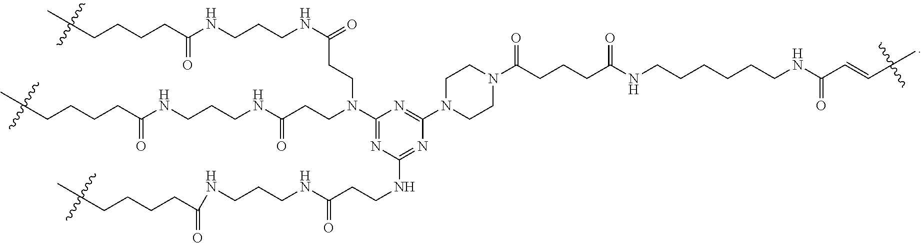 [SMILES: */C=C/C(=O)NCCCCCCNC(=O)CCCC(=O)N1CCN(c2nc(NCCC(=O)NCCCNC(=O)CCCC*)nc(N(CCC(=O)NCCCNC(=O)CCCC*)CCC(=O)NCCCNC(=O)CCCC*)n2)CC1]

In some embodiments, L^{M} is

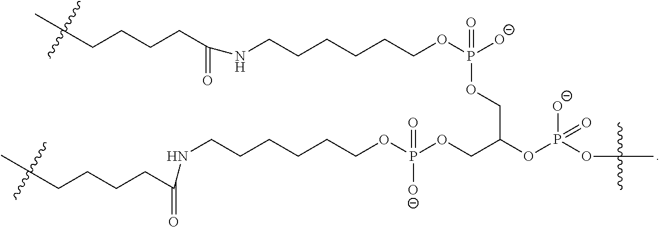 [SMILES: *CCCCC(=O)NCCCCCCOP(=O)([O-])OCC(COP(=O)([O-])OCCCCCCNC(=O)CCCC*)OP(=O)([O-])O*]

In some embodiments,

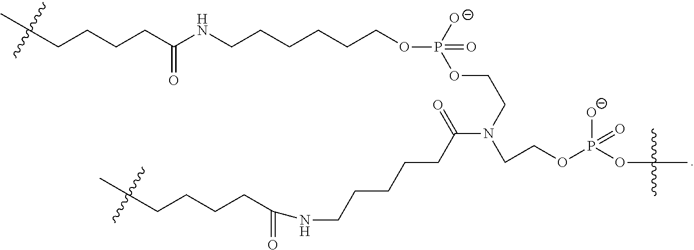 [SMILES: *CCCCC(=O)NCCCCCCOP(=O)([O-])OCCN(CCOP(=O)([O-])O*)C(=O)CCCCCNC(=O)CCCC*]

L^{M} is

In some embodiments, a carbohydrate moiety or bicyclic ketal or bicyclic ketal moiety is R^{CD}, wherein R^{CD} is an optionally substituted, linear or branched group selected from a C₁₋₁₀₀ aliphatic group and a C₁₋₁₀₀ heteroaliphatic group having 1-30 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy^{L}. In some embodiments, R^{CD} is an optionally substituted, linear or branched group selected from a C₁₋₁₀₀ aliphatic group and a C₁₋₁₀₀ heteroaliphatic group having 1-30 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are independently replaced with a tetravalent monosaccharide, disaccharide or polysaccharide moiety. In some embodiments, R^{CD} is an optionally substituted, linear or branched group selected from a C₁₋₁₀₀ aliphatic group and a C₁₋₁₀₀ heteroaliphatic group having 1-30 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are independently replaced with a tetravalent GalNac moiety, or a tetravalent moiety of a GalNac derivative.

In some embodiments, R^{CD} is optionally substituted

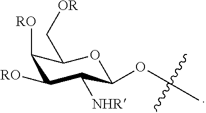

In some embodiments, R′ is —C(O)R. In some embodiments, R^{CD} is a monosaccharide moiety. In some embodiments, R^{CD} is a monovalent GalNac moiety. In some embodiments, R^{CD} is

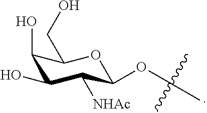

In some embodiments, R^{CD} is optionally substituted

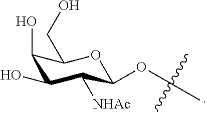

In some embodiments, R^{CD} is optionally substituted

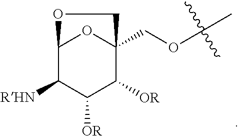

In some embodiments, R′ is —C(O)R. In some embodiments, R^{CD} is optionally substituted

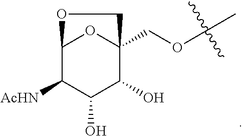 [SMILES: *OC[C@@]12CO[C@@H](O1)[C@H](NC(C)=O)[C@@H](O)[C@H]2O]

In some embodiments, R^{CD} is a disaccharide moiety. In some embodiments, R^{CD} is a polysaccharide moiety.

In some embodiments, R^{CD} has the structure of R^{G}-L-, wherein R^{G} is —H, or an optionally substituted group selected from C₃-C₂₀ cycloaliphatic, and 3-20 membered heterocyclyl having 1-10 heteroatoms independently selected from oxygen, nitrogen, sulfur, phosphorus, boron and silicon. In some embodiments, R^{CD} has the structure of R^{G}-L-, wherein R^{G} is an optionally substituted group selected from C₃-C₂₀ cycloaliphatic, and 3-20 membered heterocyclyl having 1-10 heteroatoms independently selected from oxygen, nitrogen, sulfur, phosphorus, boron and silicon. In some embodiments, R^{CD} has the structure of R^{G}-L-, wherein R^{G} is an optionally substituted group selected from C₃-C₂₀ cycloaliphatic, and 3-20 membered heterocyclyl having 1-10 heteroatoms independently selected from oxygen, nitrogen, sulfur, phosphorus, boron and silicon, wherein at least one heteroatom is oxygen. In some embodiments, R^{G} is substituted, and at least one substitute of each R^{G} is bonded to R^{G} through an oxygen atom. In some embodiments, R^{G} is substituted, and at least one substitute of each R^{G} is bonded to R^{G} through a nitrogen atom. In some embodiments, R^{G} is independently substituted, and each carbon atom of each R^{G} is independently bonded to a substituent through an oxygen or nitrogen atom. In some embodiments, R^{G} is independently substituted, and each carbon atom of each R^{G} is independently bonded to a substituent through an oxygen or nitrogen atom. In some embodiments, R^{G} is optionally substituted 3-20 membered heterocyclyl having 1-10 oxygen atoms. In some embodiments, R^{G} is optionally substituted 3-6 membered heterocyclyl having one oxygen atom. In some embodiments, each R^{G} is independently optionally substituted 3-20 membered heterocyclyl having 1-10 oxygen atoms. In some embodiments, R^{G} is independently optionally substituted 3-6 membered heterocyclyl having one oxygen atom. In some embodiments, each carbon of the heterocyclyl ring of R^{G} is independently boned to an oxygen or nitrogen atom. In some embodiments, two or more carbon atoms of the heterocyclyl ring of R^{G} are independently boned to an oxygen or nitrogen atom. In some embodiments, two or more carbon atoms of the heterocyclyl ring of R^{G} are independently boned to an oxygen or nitrogen atom. In some embodiments, three or more carbon atoms of the heterocyclyl ring of R^{G} are independently boned to an oxygen or nitrogen atom. In some embodiments, four or more carbon atoms of the heterocyclyl ring of R^{G} are independently boned to an oxygen or nitrogen atom. In some embodiments, five or more carbon atoms of the heterocyclyl ring of R^{G} are independently boned to an oxygen or nitrogen atom. In some embodiments, two or more carbon atoms of the heterocyclyl ring of R^{G} are independently boned to an oxygen atom. In some embodiments, three or more carbon atoms of the heterocyclyl ring of R^{G} are independently boned to an oxygen atom. In some embodiments, four or more carbon atoms of the heterocyclyl ring of R^{G} are independently boned to an oxygen atom. In some embodiments, five or more carbon atoms of the heterocyclyl ring of R^{G} are independently boned to an oxygen atom. In some embodiments, R^{G}—H is C₃₋₂₀ polyol comprising a —CHO or —C(O)— group.

In some embodiments, R^{CD} has the structure of R^{G}-L-, wherein R^{G} is —H, or a substituted group selected from C₃-C₂₀ cycloaliphatic, and 3-20 membered heterocyclyl having 1-10 heteroatoms independently selected from oxygen, nitrogen, sulfur, phosphorus, boron and silicon, wherein 1-20 of the substituents are R groups. In some embodiments, R^{CD} has the structure of R^{G}-L-, wherein R^{G} is —H, or a substituted group selected from C₃-C₂₀ cycloaliphatic, and 3-20 membered heterocyclyl having 1-10 heteroatoms independently selected from oxygen, nitrogen, sulfur, phosphorus, boron and silicon, wherein 1-20 of the substituents are —OR or —N(R)₂ groups. In some embodiments, R^{CD} has the structure of R^{G}-L-, wherein R^{G} is —H, or a substituted group selected from C₃-C₂₀ cycloaliphatic, and 3-20 membered heterocyclyl having 1-10 heteroatoms independently selected from oxygen, nitrogen, sulfur, phosphorus, boron and silicon, wherein 1-20 of the substituents are —OH and —N(R)₂. In some embodiments, R^{CD} has the structure of R^{G}-L-, wherein R^{G} is —H, or a substituted group selected from C₃-C₂₀ cycloaliphatic, and 3-20 membered heterocyclyl having 1-10 heteroatoms independently selected from oxygen, nitrogen, sulfur, phosphorus, boron and silicon, wherein 1-20 of the substituents are —OH and —NHR. In some embodiments, R^{CD} has the structure of R^{G}-L-, wherein R^{G} is —H, or a substituted group selected from C₃-C₂₀ cycloaliphatic, and 3-20 membered heterocyclyl having 1-10 heteroatoms independently selected from oxygen, nitrogen, sulfur, phosphorus, boron and silicon, wherein 1-20 of the substituents are —OH and —NHC(O)R.

In some embodiments, R^{G} is substituted 3-20 membered heterocyclyl having 1-10 heteroatoms independently selected from oxygen, nitrogen, sulfur, phosphorus, boron and silicon. In some embodiments, R^{G} is substituted 3-20 membered heterocyclyl having 1-10 heteroatoms independently selected from oxygen and nitrogen. In some embodiments, R^{G} is substituted 3-20 membered heterocyclyl having 1-10 oxygen. In some embodiments, R^{G} is substituted

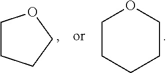 [SMILES: C1CCOC1 ; C1CCOCC1]

In some embodiments, R^{G} is substituted

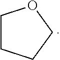

In some embodiments, R^{G} is substituted

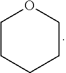

In some embodiments, R^{G} is

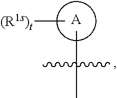

wherein each variable is independently as described in the present disclosure. In some embodiments, t is 1, 2, 3, 4, 5, 6, 7, 8, 9, or 10. In some embodiments, t is at least 1, 2, 3, 4, 5, 6, 7, 8, 9, or 10. In some embodiments, t is at least 1. In some embodiments, t is at least 2. In some embodiments, t is at least 3. In some embodiments, t is at least 4. In some embodiments, t is at least 5. In some embodiments, t is at least 6. In some embodiments, each R^{1s} is independently —OR′ or —N(R′)₂. In some embodiments, each R′ is independently —C(O)R. In some embodiments, each R^{1a} is independently —OR′ or —NHR′. In some embodiments, each R^{1s} is independently —OH or —NHR′. In some embodiments, each R^{1s} is independently —OH or —NHC(O)R. In some embodiments, Ring A is optionally substituted

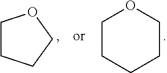 [SMILES: C1CCOC1 ; C1CCOCC1]

In some embodiments, Ring A is optionally substituted

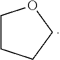

In some embodiments, Ring A is optionally substituted

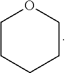

In some embodiments, R^{G} is

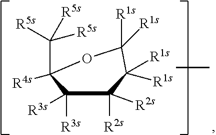

wherein each variable is independently as described in the present disclosure (i.e., R^{G}—H is

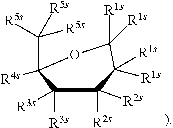

In some embodiments, at least 1, 2, 3, 4, 5, or 6 of R^{1s}, R^{2s}, R^{3s}, R^{4s} and R^{5s} are independently —OR′ or —N(R′)₂. In some embodiments, at least 1, 2, 3, 4, 5, or 6 of R^{1s}, R^{2s}, R^{3s}, R^{4s} and R^{5s} are independently —OR′ or —NHR′. In some embodiments, at least 1, 2, 3, 4, 5, or 6 of R^{1s}, R^{2s}, R^{3s}, R^{4s} and R^{5s} are independently —OH or —NHR′. In some embodiments, at least 1, 2, 3, 4, 5, or 6 of R^{1s}, R^{2s}, R^{3s}, R^{4s} and R^{5s} are independently —OH or —NHC(O)R. In some embodiments, at least 1, 2, 3, 4, 5, or 6 of R^{1s}, R^{2s}, R^{3s}, R^{4s} and R^{5s} are —OH.

In some embodiments, each ring carbon atom of the cycloaliphatic or heterocyclic ring of R^{G} is independently substituted. In some embodiments, no more than 1, 2, 3, 4, 5, 6, 7, 8, 9, or 10 ring carbon atoms of the cycloaliphatic or heterocyclic ring of R^{G} are not substituted. In some embodiments, no more than 1 ring carbon atom is not substituted. In some embodiments, no more than 2 ring carbon atoms are not substituted. In some embodiments, no more than 3 ring carbon atoms are not substituted. In some embodiments, no more than 4 ring carbon atoms are not substituted. In some embodiments, no more than 5 ring carbon atoms are not substituted. In some embodiments, no more than 6 ring carbon atoms are not substituted. In some embodiments, no more than 7 ring carbon atoms are not substituted. In some embodiments, no more than 8 ring carbon atoms are not substituted. In some embodiments, no more than 9 ring carbon atoms are not substituted. In some embodiments, no more than 10 ring carbon atoms are not substituted. In some embodiments, no more than 1, 2, 3, 4, 5, 6, 7, 8, 9, or 10 ring carbon atoms of the cycloaliphatic or heterocyclic ring of R^{G} are not substituted with —OH or —N(R′)₂. In some embodiments, no more than 1 ring carbon atom is not substituted with —OH or —N(R′)₂. In some embodiments, no more than 2 ring carbon atoms are not substituted with —OH or —N(R′)₂. In some embodiments, no more than 3 ring carbon atoms are not substituted with —OH or —N(R′)₂. In some embodiments, no more than 4 ring carbon atoms are not substituted with —OH or —N(R′)₂. In some embodiments, no more than 5 ring carbon atoms are not substituted with —OH or —N(R′)₂. In some embodiments, no more than 6 ring carbon atoms are not substituted with —OH or —N(R′)₂. In some embodiments, no more than 7 ring carbon atoms are not substituted with —OH or —N(R′)₂. In some embodiments, no more than 8 ring carbon atoms are not substituted with —OH or —N(R′)₂. In some embodiments, no more than 9 ring carbon atoms are not substituted with —OH or —N(R′)₂. In some embodiments, no more than 10 ring carbon atoms are not substituted with —OH or —N(R′)₂. In some embodiments, no more than 1, 2, 3, 4, 5, 6, 7, 8, 9, or 10 ring carbon atoms of the cycloaliphatic or heterocyclic ring of R^{G} are not substituted with —OH. In some embodiments, no more than 1 ring carbon atom is not substituted with —OH. In some embodiments, no more than 2 ring carbon atoms are not substituted with —OH. In some embodiments, no more than 3 ring carbon atoms are not substituted with —OH. In some embodiments, no more than 4 ring carbon atoms are not substituted with —OH. In some embodiments, no more than 5 ring carbon atoms are not substituted with —OH. In some embodiments, no more than 6 ring carbon atoms are not substituted with —OH. In some embodiments, no more than 7 ring carbon atoms are not substituted with —OH. In some embodiments, no more than 8 ring carbon atoms are not substituted with —OH. In some embodiments, no more than 9 ring carbon atoms are not substituted with —OH. In some embodiments, no more than 10 ring carbon atoms are not substituted with —OH. In some embodiments, at least 10%, 20%, 30%, 40%, 50%, 60%, 70%, 80%, 90%, or 95% percent of the ring carbon atoms of the cycloaliphatic or heterocyclic ring of R^{G} are substituted with —OH or —N(R′)₂. In some embodiments, no more than 10%, 20%, 30%, 40%, 50%, 60%, 70%, 80%, 90%, or 95% of the ring carbon atoms of the cycloaliphatic or heterocyclic ring of R^{G} are not substituted with —OH. In some embodiments, no more than 10% of the ring carbon atoms are not substituted with —OH. In some embodiments, no more than 20% of the ring carbon atoms are not substituted with —OH. In some embodiments, each ring carbon atom of the cycloaliphatic or heterocyclic ring of R^{G} is independently substituted. In some embodiments, at least 1, 2, 3, 4, 5, 6, 7, 8, 9, or 10 ring carbon atoms of the cycloaliphatic or heterocyclic ring of R^{G} are substituted. In some embodiments, at least 1 ring carbon atom is substituted. In some embodiments, at least 2 ring carbon atoms are substituted. In some embodiments, at least 3 ring carbon atoms are substituted. In some embodiments, at least 4 ring carbon atoms are substituted. In some embodiments, at least 5 ring carbon atoms are substituted. In some embodiments, at least 6 ring carbon atoms are substituted. In some embodiments, at least 7 ring carbon atoms are substituted. In some embodiments, at least 8 ring carbon atoms are substituted. In some embodiments, at least 9 ring carbon atoms are substituted. In some embodiments, at least 10 ring carbon atoms are substituted. In some embodiments, at least 1, 2, 3, 4, 5, 6, 7, 8, 9, or 10 ring carbon atoms of the cycloaliphatic or heterocyclic ring of R^{G} are substituted with —OH or —N(R′)₂. In some embodiments, at least 1 ring carbon atom is substituted with —OH or —N(R′)₂. In some embodiments, at least 2 ring carbon atoms are substituted with —OH or —N(R′)₂. In some embodiments, at least 3 ring carbon atoms are substituted with —OH or —N(R′)₂. In some embodiments, at least 4 ring carbon atoms are substituted with —OH or —N(R′)₂. In some embodiments, at least 5 ring carbon atoms are substituted with —OH or —N(R′)₂. In some embodiments, at least 6 ring carbon atoms are substituted with —OH or —N(R′)₂. In some embodiments, at least 7 ring carbon atoms are substituted with —OH or —N(R′)₂. In some embodiments, at least 8 ring carbon atoms are substituted with —OH or —N(R′)₂. In some embodiments, at least 9 ring carbon atoms are substituted with —OH or —N(R′)₂. In some embodiments, at least 10 ring carbon atoms are substituted with —OH or —N(R′)₂. In some embodiments, at least 1, 2, 3, 4, 5, 6, 7, 8, 9, or 10 ring carbon atoms of the cycloaliphatic or heterocyclic ring of R^{G} are substituted with —OH. In some embodiments, at least 1 ring carbon atom is substituted with —OH. In some embodiments, at least 2 ring carbon atoms are substituted with —OH. In some embodiments, at least 3 ring carbon atoms are substituted with —OH. In some embodiments, at least 4 ring carbon atoms are substituted with —OH. In some embodiments, at least 5 ring carbon atoms are substituted with —OH. In some embodiments, at least 6 ring carbon atoms are substituted with —OH. In some embodiments, at least 7 ring carbon atoms are substituted with —OH. In some embodiments, at least 8 ring carbon atoms are substituted with —OH. In some embodiments, at least 9 ring carbon atoms are substituted with —OH. In some embodiments, at least 10 ring carbon atoms are substituted with —OH. In some embodiments, at least 10%, 20%, 30%, 40%, 50%, 60%, 70%, 80%, 90%, or 95% percent of the ring carbon atoms of the cycloaliphatic or heterocyclic ring of R^{G} are substituted with —OH or —N(R′)₂. In some embodiments, at least 10% the ring carbon atoms are substituted with —OH or —N(R′)₂. In some embodiments, at least 20% the ring carbon atoms are substituted with —OH or —N(R′)₂. In some embodiments, at least 30% of the ring carbon atoms are substituted with —OH or —N(R′)₂. In some embodiments, at least 40% of the ring carbon atoms are substituted with —OH or —N(R′)₂. In some embodiments, at least 50% of the ring carbon atoms are substituted with —OH or —N(R′)₂. In some embodiments, at least 60% of the ring carbon atoms are substituted with —OH or —N(R′)₂. In some embodiments, at least 70% of the ring carbon atoms are substituted with —OH or —N(R′)₂. In some embodiments, at least 80% of the ring carbon atoms are substituted with —OH or —N(R′)₂. In some embodiments, at least 90% of the ring carbon atoms are substituted with —OH or —N(R′)₂. In some embodiments, at least 95% of the ring carbon atoms are substituted with —OH or —N(R′)₂. In some embodiments, at least 10%, 20%, 30%, 40%, 50%, 60%, 70%, 80%, 90%, or 95% of the ring carbon atoms of the cycloaliphatic or heterocyclic ring of R^{G} are substituted with —OH. In some embodiments, at least 10% of the ring carbon atoms are substituted with —OH. In some embodiments, at least 20% of the ring carbon atoms are substituted with —OH. In some embodiments, at least 30% of the ring carbon atoms are substituted with —OH. In some embodiments, at least 40% of the ring carbon atoms are substituted with —OH. In some embodiments, at least 50% of the ring carbon atoms are substituted with —OH. In some embodiments, at least 60% of the ring carbon atoms are substituted with —OH. In some embodiments, at least 70% of the ring carbon atoms are substituted with —OH. In some embodiments, at least 80% of the ring carbon atoms are substituted with —OH. In some embodiments, at least 90% of the ring carbon atoms are substituted with —OH. In some embodiments, at least 95% of the ring carbon atoms are substituted with —OH. In some embodiments, at least one ring carbon atom is substituted with —N(R′)₂. In some embodiments, at least one ring carbon atom is substituted with —NHC(O)R. In some embodiments, at least one ring carbon atom is substituted with —NHC(O)R, wherein R is optionally substituted C₁₋₆ aliphatic. In some embodiments, at least one ring carbon atom is substituted with —NHAc.

In some embodiments, R^{G} is optionally substituted

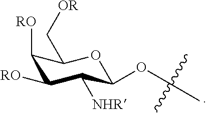

In some embodiments, R′ is —C(O)R. In some embodiments, R^{G} is

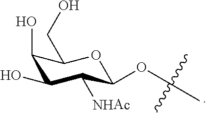

In some embodiments, R^{G} is optionally substituted

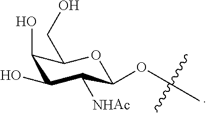

In some embodiments, R^{G} is optionally substituted

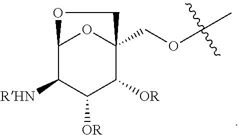

In some embodiments, R′ is —C(O)R. In some embodiments, R^{G} is optionally substituted

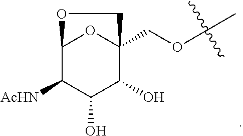 [SMILES: *OC[C@@]12CO[C@@H](O1)[C@H](NC(C)=O)[C@@H](O)[C@H]2O]

In some embodiments, R^{CD}, or R^{G}, is of such a structure that R^{CD}—H, or R^{G}—H, is

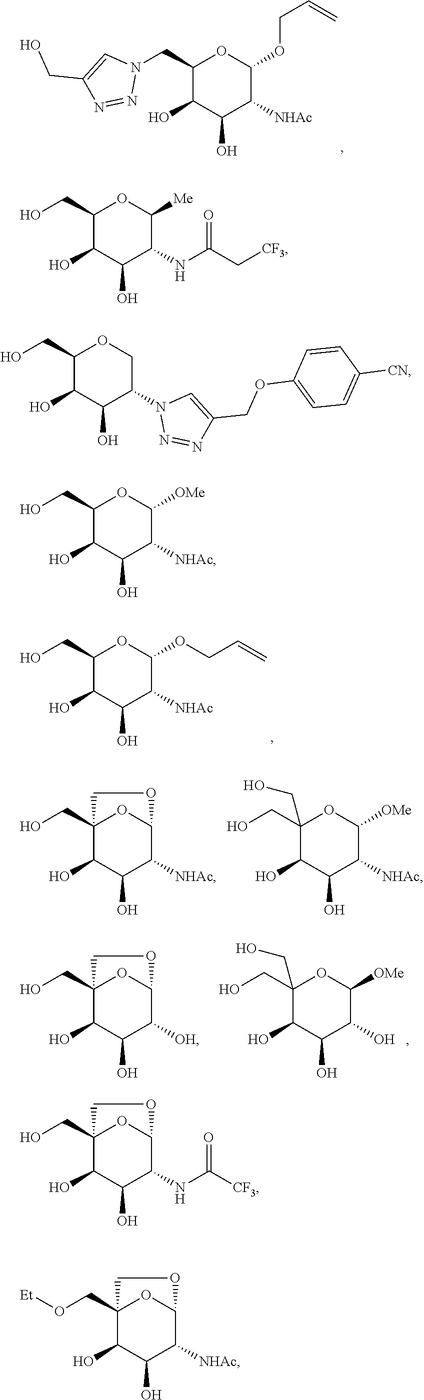

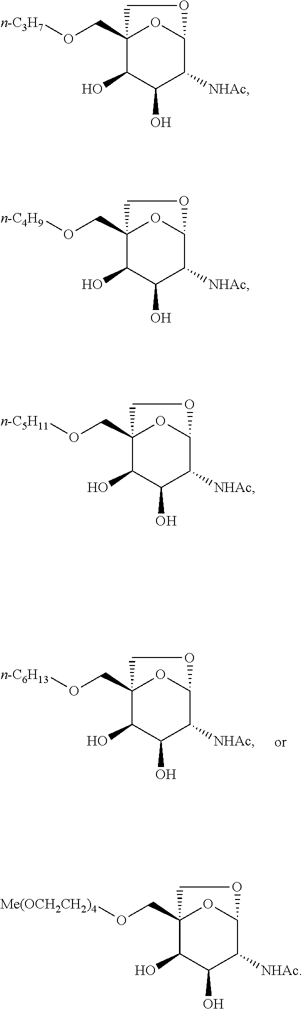

In some embodiments, R^{CD}, or R^{G}, is of such a structure that R^{CD}—H, or R^{G}—H, is a ligand for the asialoglycoprotein receptor (ASGPR). Various other ASGPR ligands are known in the art and can be utilized in accordance with the present disclose. In some embodiments, carbohydrate moieties described in are useful for targeted delivery of provided oligonucleotides to liver.

In some embodiments, L is a covalent bond. In some embodiments, L is bivalent optionally substituted C₁₋₆ aliphatic wherein one or more methylene units are independently and optionally replaced with —O—. In some embodiments, L is —O—CH₂—.

In some embodiments, R^{CD} is an oligomeric or polymeric moiety of R^{G}—H, wherein each R^{G} is independently as described in the present disclosure.

In some embodiments, an APOC3 oligonucleotide or single-stranded RNAi agent comprises any targeting moiety described herein or known in the art. In some embodiments, an APOC3 oligonucleotide is a single-stranded RNAi agent.

In some embodiments, a targeting moiety is a ligand for the asialoglycoprotein receptor (ASGPR).

In some embodiments, a targeting moiety is a ligand for the asialoglycoprotein receptor (ASGPR) disclosed in: Sanhueza et al. J. Am. Chem. Soc., 2017, 139 (9), pp 3528-3536.

In some embodiments, a targeting moiety is a ligand for the asialoglycoprotein receptor (ASGPR) disclosed in Liras et al. US 20160207953.

In some embodiments, a targeting moiety is a substituted-6,8-dioxabicyclo[3.2.1]octane-2,3-diol derivative disclosed in Liras et al. US 20160207953.

In some embodiments, a targeting moiety is a ligand for the asialoglycoprotein receptor (ASGPR) disclosed in Liras et al. US 20150329555.

In some embodiments, a targeting moiety is a substituted-6,8-dioxabicyclo[3.2.1]octane-2,3-diol derivative disclosed in Liras et al. US 20150329555.

In some embodiments, an additional chemical moiety conjugated to an APOC3 oligonucleotide or single-stranded RNAi agent is a GalNAc moiety. In some embodiments, an additional chemical moiety conjugated to an APOC3 oligonucleotide or single-stranded RNAi agent is a GalNAc moiety which is conjugated at any position.

In some embodiments, an additional chemical moiety conjugated to an APOC3 oligonucleotide or single-stranded RNAi agent is a GalNAc moiety, conjugated via a linker to a 5′-H T. In some embodiments, an additional chemical moiety conjugated to an APOC3 oligonucleotide or single-stranded RNAi agent is a GalNAc moiety, conjugated via a linker to a 5′-H T which is conjugated at any position.

In some embodiments, an additional chemical moiety is GaNC6T (also known as TGaNC6T, or conjugation of a GalNAc moiety to 5′H T via amino C6 linker) at any position.

In some embodiments, an additional chemical moiety is GaNC6T, e.g., conjugation of a GalNAc moiety to 5′H T via amino C6 linker (e.g., at the penultimate or antepenultimate nucleotide [counting 5′ to 3′]; for example, the 5′ nucleotide of the 3′-terminal dinucleotide (e.g., the 5′ nucleotide of the 3′-terminal dinucleotide is, of the two nucleotides of the 3′-terminal dinucleotide, the nucleotide closer to the 5′-end of the oligonucleotide) or the nucleotide immediately 5′ to the 5′ nucleotide of the 3′-terminal dinucleotide:

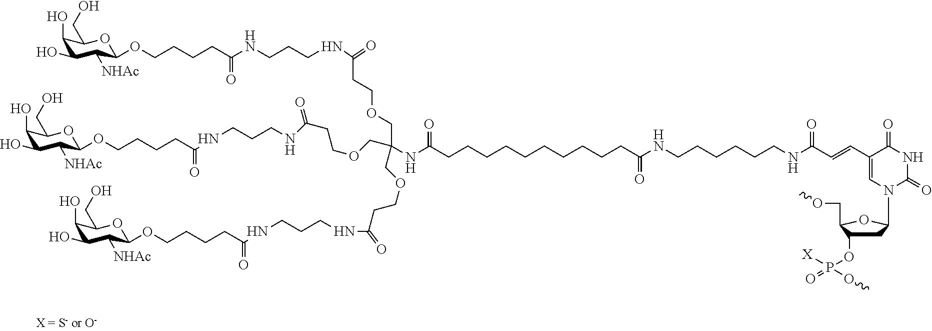

In some embodiments or single-stranded RNAi agent, the oligonucleotide or single-stranded RNAi agent comprises a linker conjugating a GalNAc moiety to the oligonucleotide or single-stranded RNAi agent. In some embodiments or single-stranded RNAi agent, the oligonucleotide or single-stranded RNAi agent comprises a linker conjugating a GalNAc moiety to the oligonucleotide or single-stranded RNAi agent, wherein the linker is attached at the 2′ position of a sugar. In some embodiments or single-stranded RNAi agent, the oligonucleotide or single-stranded RNAi agent comprises a linker conjugating a GalNAc moiety to the oligonucleotide or single-stranded RNAi agent, wherein the linker is attached to a base. In some embodiments or single-stranded RNAi agent, the oligonucleotide or single-stranded RNAi agent comprises a linker conjugating a GalNAc moiety to the oligonucleotide or single-stranded RNAi agent, wherein the linker is attached to a T base. In some embodiments or single-stranded RNAi agent, the oligonucleotide or single-stranded RNAi agent comprises a linker conjugating a GalNAc moiety to the oligonucleotide or single-stranded RNAi agent.

In some embodiments, a linker attaching a GalNAc moiety is a biocleavable linker. Such a linker allows the intracellular removal of the GalNAc moiety, so that the GalNAc moiety will not interfere with Ago-2 activity or RNA interference.

In some embodiments, a GalNAc moiety is conjugated to an APOC3 oligonucleotide or single-stranded RNAi agent at the penultimate or antepenultimate nucleotide.

In some embodiments, a GalNAc moiety can be conjugated at the penultimate nucleotide of a single-stranded RNAi agent (the more 5′ position of a 3′-terminal dinucleotide), or at the antepenultimate nucleotide of a single-stranded RNAi agent (the nucleotide immediate 5′ to the 3′-terminal dinucleotide). Without wishing to be bound by any particular theory, this disclosure suggests that the penultimate or antepenultimate nucleotide of a single-stranded RNAi agent (e.g., the more 5′ position of a 3′-terminal dinucleotide) can be adjacent to a pocket in Ago-2, and a GalNAc moiety may be capable of insertion into said pocket, such that the GalNAc moiety does not interfere with Ago-2 activity. Without wishing to be bound by any particular theory, this disclosure suggests that if a GalNAc moiety is attached at the penultimate or antepenultimate nucleotide, it may thus not be necessary to cleave the GalNAc moiety to allow RNAi activity, and it may thus be acceptable to use a more robust, non-biocleavable linker to attach a GalNAc moiety to the oligonucleotide or single-stranded RNAi agent. The more robust linker thus is less susceptible to cleavage, increasing the probability that a GalNAc moiety will increase delivery of the oligonucleotide or single-stranded RNAi agent.

In some embodiments, the GalNAc moiety is attached via a AMC6 linker.

In some embodiments, the GalNAc moiety is attached via a AMC6 linker attached at a T base (AMC6T).

In some embodiments, AMC6T has a structure of:

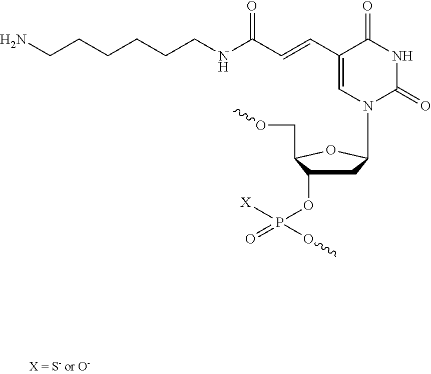

In some non-limiting examples, AMC6T is either the penultimate or antepenultimate nucleotide [counting 5′ to 3′]; for example, the 5′ nucleotide of the 3′-terminal dinucleotide, or the nucleotide immediately 5′ to the 5′ nucleotide of the 3′-terminal dinucleotide.

In some embodiments of a single-stranded RNAi agent, the single-stranded RNAi agent comprises a AMC6T at the penultimate or antepenultimate nucleotide.

As disclosed herein, GaNC6T is a component in an efficacious single-stranded RNAi agent. In some non-limiting examples, GaNC6T is either at the penultimate or antepenultimate nucleotide [counting 5′ to 3′]; for example, the 5′ nucleotide of the 3′-terminal dinucleotide, or the nucleotide immediately 5′ to the 5′ nucleotide of the 3′-terminal dinucleotide. In some non-limiting examples disclosed herein, GaNC6T is at nucleotide position 20 out of 21 (counting from the 5′-end), or 24 out of 25 (counting from the 5′-end).

In some embodiments, an APOC3 oligonucleotide or single-stranded RNAi agent is conjugated to Tri-antennary GalNAc Acid (e.g., via a C10, C3 or triazine linker):

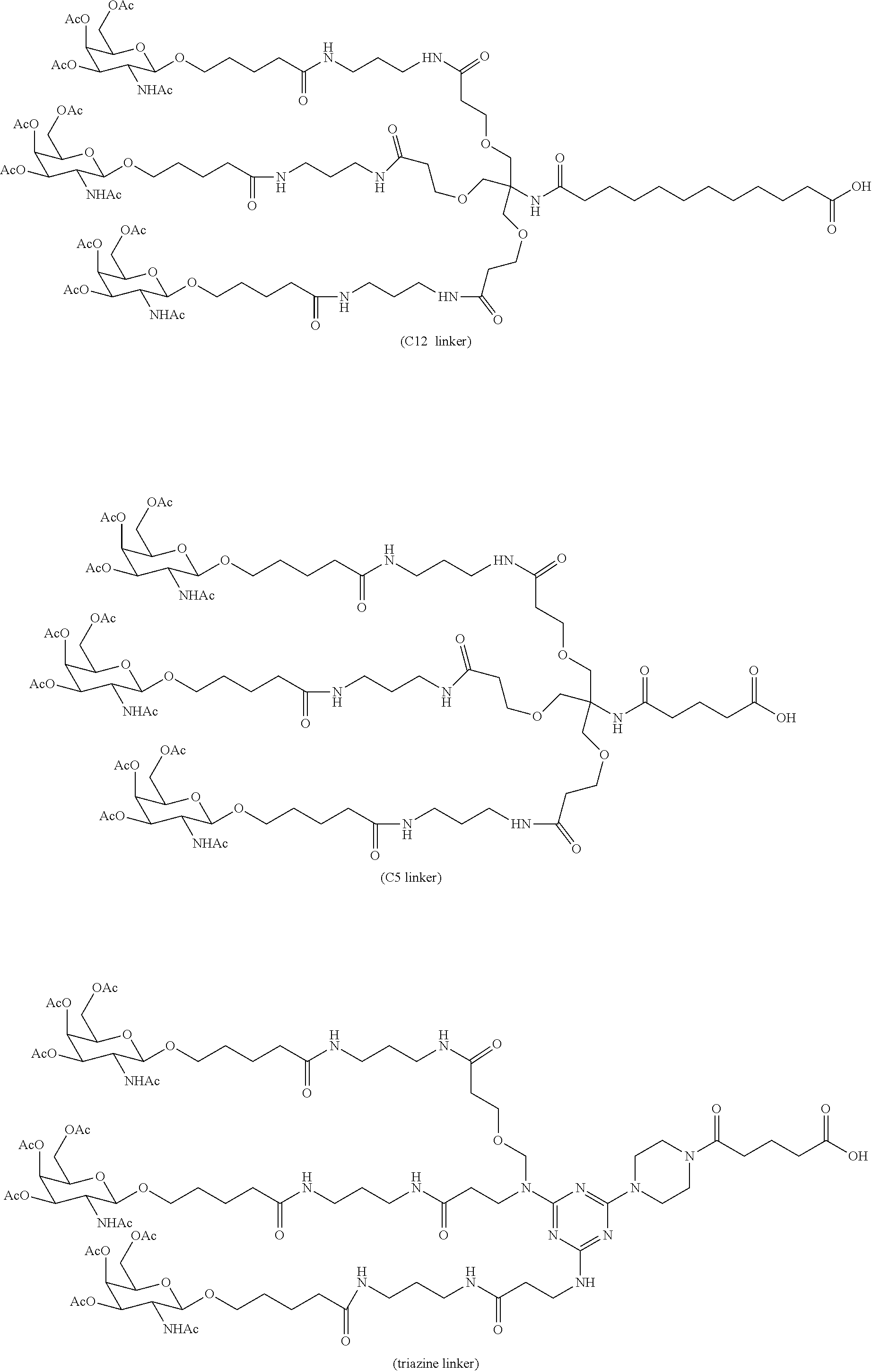

These structures represent the protected versions, as they comprise —OAc (—O-acetate groups). In some embodiments, the Ac groups are removed during de-protection following conjugation of the compound to the oligonucleotide. In some embodiments, de-protection is performed with concentrated ammonium hydroxide, e.g., as described in Example 37B. In the de-protected versions of these structures, —OAc is replaced by —OH.

In some embodiments, a GalNAc moiety is conjugated at the 5′-end. Each of these additional chemical moieties (Tri-antennary GalNAc Acid, with each of the C12, C5 or triazine linkers) was conjugated to an APOC3 oligonucleotide targeting Factor XI (FXI), which operates via a RNase H mechanism.

Several oligonucleotides were constructed; each comprises an APOC3 oligonucleotide targeting Factor XI (FXI), which operates via a RNase H mechanism, with each conjugated to a different Tri-antennary GalNAc Acid, with each of the C12, C5 or triazine linkers. The Tri-antennary GalNAc Acid has been revealed experimentally (data not shown) to improve the delivery of the oligonucleotides to the liver.

In some embodiments, the present disclosure pertains to any oligonucleotide conjugated to Tri-antennary GalNAc Acid. In some embodiments, the present disclosure pertains to any oligonucleotide conjugated to Tri-antennary GalNAc Acid via a C10, C3 or triazine linker. In some embodiments, the present disclosure pertains to any oligonucleotide conjugated to Tri-antennary GalNAc Acid, wherein the oligonucleotide directs knockdown of a target transcript mediated by a RNase H or RNA interference mechanism. In some embodiments, the present disclosure pertains to any oligonucleotide conjugated to Tri-antennary GalNAc Acid via a C10, C3 or triazine linker, wherein the oligonucleotide directs knockdown of a target transcript mediated by a RNase H or RNA interference mechanism.

In some embodiments, the present disclosure pertains to any oligonucleotide conjugated to Tri-antennary GalNAc Acid, wherein the oligonucleotide directs knockdown of a target transcript mediated by a RNase H or RNA interference mechanism, wherein the RNA interference mechanism is directed by a RNAi agent comprising 1, 2 or more strands. In some embodiments, the present disclosure pertains to any oligonucleotide conjugated to Tri-antennary GalNAc Acid via a C10, C3 or triazine linker, wherein the oligonucleotide directs knockdown of a target transcript mediated by a RNase H or RNA interference mechanism, wherein the RNA interference mechanism is directed by a RNAi agent comprising 1, 2 or more strands.

In addition, the present disclosure shows that in provided oligonucleotides capable of directing single-stranded RNA interference it is not necessary for the first nucleotide on the 5′-end of a single-stranded RNAi agent to match the corresponding portion of the sequence of the target transcript.

Oligonucleotides capable of directing single-stranded RNA interference were prepared and characterized using a variety of methods in accordance of the present disclosure. In some embodiments, a provided oligonucleotide composition is a single-stranded RNAi agent of an APOC3 oligonucleotide type listed in Table 1A. In some embodiments, a provided oligonucleotide composition is a single-stranded RNAi agent of an APOC3 oligonucleotide type listed as any of Formats illustrated in FIG. 1.

In some embodiments, an APOC3 oligonucleotide is capable of directing knockdown of a target transcript by both RNase H-mediated knockdown and RNA interference. Such an APOC3 oligonucleotide is described herein a dual mechanism or hybrid oligonucleotide.

In some embodiments, an APOC3 oligonucleotide, an APOC3 oligonucleotide that directs RNA interference, an APOC3 oligonucleotide that directs RNase H-mediated knockdown, or an APOC3 oligonucleotide that directs both RNA interference and RNase H-mediated knockdown can comprise any additional chemical moiety, including but not limited to, any GalNAc moiety described or referenced in any of: U.S. Pat. Nos. 5,382,524; 5,491,075; 5,545,553; 5,705,367; 5,733,765; 5,786,184; 5,798,233; 5,854,042; 5,871,990; 5,945,322; 6,165,469; 6,187,310; 6,342,382; 6,465,220; 6,503,744; 6,699,705; 6,723,545; 6,780,624; 6,825,019; 6,905,867; 6,911,337; 7,026,147; 7,078,207; 7,138,258; 7,166,717; 7,169,593; 7,169,914; 7,189,836; 7,192,756; 7,202,353; 7,208,304; 7,211,657; 7,217,549; 7,220,848; 7,238,509; 7,338,932; 7,371,838; 7,384,771; 7,462,474; 7,598,068; 7,608,442; 7,682,787; 7,723,092; 8,039,218; 8,137,941; 8,268,596; 8,871,723; or 9,222,080.

Dual Mechanism or Hybrid Oligonucleotide

In some embodiments, an APOC3 oligonucleotide or an APOC3 oligonucleotide that directs both RNA interference and RNase H-mediated knockdown can comprise structural element of any oligonucleotide described herein or known in the art.

As disclosed herein, some oligonucleotides are capable of directing knockdown of a transcript target by both RNase H-mediated knockdown and RNA interference.

As disclosed herein, some oligonucleotides (including but not limited to those described herein as dual mechanism or hybrid oligonucleotides or hybrid RNAi agents) are capable of directing knockdown of a transcript target by both RNase H-mediated knockdown and RNA interference.

Wishing wishing to be bound by any particular theory, the present disclosure suggests that a hybrid oligonucleotide can have particular advantages to either an APOC3 oligonucleotide capable of directing knockdown solely by RNase H-mediated knockdown, or an APOC3 oligonucleotide capable of directing knockdown solely by RNA interference. For example, if several hybrid oligonucleotides are introduced into the same cell, some but not all hybrid oligonucleotide may participate in the RISC pathway; those which do not are available to participate in the RNase H-mediated pathway. For example, if several hybrid oligonucleotides are introduced into the same cell, some but not all hybrid oligonucleotide may participate in the RNase H-mediated pathway; those which do not are available to participate in the RISC pathway. Without wishing to be bound by any particular theory, the present disclosure suggests that a hybrid oligonucleotide may be able to mediate more efficacious knockdown than an APOC3 oligonucleotide capable of directing knockdown solely by RNase H-mediated knockdown, or an APOC3 oligonucleotide capable of directing knockdown solely by RNA interference, as the hybrid oligonucleotide is capable of directing knockdown via both pathways. In at least some cells, levels of RNase H activity and RNA interference may differ from cell compartment to cell compartment. In some embodiments, a hybrid oligonucleotide can direct knockdown in various cell compartments via RNase H-mediated knockdown or RNA interference. In some embodiments, if RNase H is saturated with oligonucleotides, the excess oligonucleotides can be available for RNA interference-mediated knockdown. In some embodiments, if Ago-2 is saturated with oligonucleotides, the excess oligonucleotides can be available for RNase H-mediated knockdown.

In some embodiments, a hybrid oligonucleotide comprises a structure which allows both knockdown via RNase H-mediated knockdown and knockdown via RNA interference.

Reportedly, RNase H and RNAi both involve knockdown of a target mRNA, but they involve different mechanisms. Reportedly, RNase H naturally involves a single-stranded DNA molecule which binds to a mRNA target and decreases expression by either sterically hindering translation, or by the RNA/DNA duplex acting as a substrate for RNase H, which cleaves the mRNA target.

In contrast, reportedly, RNAi naturally involves a double-stranded RNA molecule, naturally produced by Dicer with two 3′ overhangs, including an antisense and a sense strand. The strands are separated as the duplex is unwound and the antisense incorporated into the RISC (RNA interference silencing complex), including Argonaute-2. The antisense strand acts as a guide for RISC to identity the complementary mRNA target and cleave it. As shown herein, certain formats of single-stranded RNAi agents are also efficacious, although single-stranded RNAi agents are not naturally produced by Dicer.

Reportedly, RNase H and RISC naturally prefer two structurally distinct types of molecules. RNase H naturally uses a single-stranded DNA molecule to target the mRNA target, forming a DNA/RNA duplex. Reportedly, RISC reportedly naturally uses a single-stranded RNA antisense strand to target the mRNA target, forming a RNA/RNA duplex. Crooke et al. 1995 Biochem. J. 312: 599-608; and Elbashir et al. Nature 2001 411: 494.

Crooke et al. 1995 Biochem. J. 312: 599-608 also reported that E. coli RNase H1 had been crystallized and studied, and that the preferred substrate was reportedly a RNA/DNA duplex. In the DNA strand, 2′-modifications such as 2′-OMe and 2′-F reportedly reduced or eliminated RNase H activity. In addition, for RNA interference, full replacement of RNA by DNA reportedly abolishes RNA interference activity of double-stranded RNAi agents. Elbashir et al. 2001 EMBO J. 20: 6877-6888. Thus, reportedly, RNase H-mediated knockdown reportedly requires a span of DNA (2′-deoxy), while RNA interference can be abolished by replacement of a span of nucleotides with DNA (2′-deoxy).

In contrast, as shown herein, 2′-OMe and 2′-F modifications are highly suitable for single-stranded RNAi agents. The Applicants thus designed and constructed several oligonucleotides which comprise (a) a seed region comprising 2′-modified nucleotides; and (b) a post-seed region comprising a stretch of 2′-deoxy (2′-deoxy) nucleotides. These are shown herein to function via both the RNAi and RNase H-mediated knockdown mechanisms.

In some embodiments, a hybrid oligonucleotide comprises a (a) seed region capable of annealing to a first complementary target mRNA region and mediating RNA interference; and (b) a post-seed region comprising a 2′-deoxy (2′-deoxy) region capable of annealing a second complementary target mRNA region and directing RNase H-mediated knockdown. The seed region can optionally comprise RNA or a modified nucleotide, e.g., with a 2′ modification (including but not limited to 2′-F, 2′-OMe and 2′-MOE), wherein the RNA or modified nucleotide comprise a natural sugar and/or a natural base, and/or a modified base, and/or an internucleotidic linkage.

A minimum length for a DNA (2′-deoxy) region efficacious for RNase H-mediated knockdown, in at least some cases, is reported to be about 5 consecutive DNA (2′-deoxy); this minimum deoxy length reportedly correlated with the minimum length required for efficient RNase H activation in vitro using partially purified mammalian RNase H enzyme. Monia et al. 1993 JBC 268: 14514-14522.

In some embodiments, a hybrid oligonucleotide comprises a (a) seed region capable of annealing to a first complementary target mRNA region; and (b) a post-seed region comprising a 2′-deoxy region, wherein the hybrid oligonucleotide is capable of directing both RNA interference and RNase H-mediated knockdown, wherein the 2′-deoxy region comprises at least 5 consecutive 2′-deoxy. In some embodiments, the 2′-deoxy can be DNA, or a modified nucleotide, e.g., a modified nucleotide with a 2′-deoxy, wherein the DNA or modified nucleotide comprise a natural sugar and/or a natural base, and/or a modified base, and/or any internucleotidic linkage. In some embodiments, the 2′-deoxy region comprises a stretch of consecutive nucleotides, wherein each nucleotide is 2′-deoxy and each internucleotidic linkage is a phosphorothioate. In some embodiments, the 2′-deoxy region comprises a stretch of consecutive nucleotides of at least 5, 6, 7, 8, 9, 10, 11, 12, 13, 14, 15, 16, 17, 18, 19, 20 or 21 nucleotides, wherein each nucleotide is 2′-deoxy and each internucleotidic linkage is a phosphorothioate.

In some embodiments, a hybrid oligonucleotide comprises a (a) seed region capable of annealing to a first complementary target mRNA region; and (b) a post-seed region comprising a 2′-deoxy region, wherein the hybrid oligonucleotide is capable of directing both RNA interference and RNase H-mediated knockdown, wherein the 2′-deoxy region comprises at least 5 consecutive 2′-deoxy. In some embodiments, the 2′-deoxy can be DNA, or a modified nucleotide, e.g., a modified nucleotide with a 2′-deoxy, wherein the DNA or modified nucleotide comprise a natural sugar and/or a natural base, and/or a modified base, and/or any internucleotidic linkage. In some embodiments, the 2′-deoxy region comprises or is a span of consecutive nucleotides, wherein each nucleotide is 2′-deoxy and each internucleotidic linkage is a phosphorothioate. In some embodiments, the 2′-deoxy region comprises or is a stretch of consecutive nucleotides, wherein each nucleotide is 2′-deoxy and each internucleotidic linkage is a phosphorothioate. In some embodiments, the 2′-deoxy region comprises a stretch of consecutive nucleotides of at least 5, 6, 7, 8, 9, 10, 11, 12, 13, 14, 15, 16, 17, 18, 19, 20 or 21 nucleotides, wherein each nucleotide is 2′-deoxy and each internucleotidic linkage is a phosphorothioate.

Without wishing to be bound by any particular theory, the present disclosure notes that WO 2015/107 425 has reported that cleavage mediated by RNase H can be modulated by the arrangement of chiral centers in phosphorothioates in an antisense oligonucleotide directing RNase H cleavage. For example, the placement of a single Rp flanked by at least 2 or 3 Sp can alter the cleavage pattern, such that the number of cleavage sites is reduced and the site of RNase H-mediated cleavage is controlled.

In some embodiments, a hybrid oligonucleotide comprises a (a) seed region capable of annealing to a first complementary target mRNA region; and (b) a post-seed region comprising a 2′-deoxy region, wherein the hybrid oligonucleotide is capable of directing both RNA interference and RNase H-mediated knockdown, wherein the 2′-deoxy region comprises a stretch of consecutive nucleotides of at least 5, 6, 7, 8, 9, 10, 11, 12, 13, 14, 15, 16, 17, 18, 19, 20 or 21 nucleotides, wherein each nucleotide is 2′-deoxy and each internucleotidic linkage is a phosphorothioate.

In some embodiments, a hybrid oligonucleotide comprises a (a) seed region capable of annealing to a first complementary target mRNA region; and (b) a post-seed region comprising a 2′-deoxy region, wherein the hybrid oligonucleotide is capable of directing both RNA interference and RNase H-mediated knockdown, wherein the 2′-deoxy region comprises a stretch of consecutive nucleotides of at least 9 nucleotides, wherein each nucleotide is 2′-deoxy and each internucleotidic linkage is a phosphorothioate.

In some embodiments, the first and second complementary target mRNA regions are regions of the same target mRNA.

In some embodiments, the first and second complementary target mRNA regions are regions of different target mRNAs.

In some embodiments of a hybrid oligonucleotide, a seed region comprises a DNA region capable of annealing to a complementary target mRNA region and directing RNase H-mediated knockdown.

Without wishing to be bound by any particular theory, the present disclosure notes that, in many cases, RNase H cleaves a single-stranded RNA target which is bound to a single-stranded DNA. In some embodiments, a hybrid oligonucleotide comprises a single-stranded 2′-deoxy portion, which is capable of binding to a target RNA transcript, forming a substrate for RNase H. In some embodiments, a hybrid oligonucleotide comprises a single-stranded 2′-deoxy portion (which comprises internucleotidic linkages which can be any internucleotidic linkage described herein or known in the art), which is capable of binding to a target RNA transcript, forming a substrate for RNase H.

In some embodiments of a single-stranded RNAi agent, a post-seed region comprises: a sequence of nucleotides comprising at least 4 consecutive 2′-deoxy. In some embodiments of a single-stranded RNAi agent, a post-seed region comprises: a sequence of nucleotides comprising at least 5 consecutive 2′-deoxy. In some embodiments of a single-stranded RNAi agent, a post-seed region comprises: a sequence of nucleotides comprising at least 6 consecutive 2′-deoxy. In some embodiments of a single-stranded RNAi agent, a post-seed region comprises: a sequence of nucleotides comprising at least 7 consecutive 2′-deoxy. In some embodiments of a single-stranded RNAi agent, a post-seed region comprises: a sequence of nucleotides comprising at least 8 consecutive 2′-deoxy. In some embodiments of a single-stranded RNAi agent, a post-seed region comprises: a sequence of nucleotides comprising at least 9 consecutive 2′-deoxy. In some embodiments of a single-stranded RNAi agent, a post-seed region comprises: a sequence of nucleotides comprising at least 10 to 20 consecutive 2′-deoxy.

In some embodiments of a hybrid oligonucleotide, a post-seed region comprises: at least 9 consecutive 2′-deoxy. In some embodiments of a single-stranded RNAi agent, a post-seed region comprises: at least 10 consecutive 2′-deoxy. The ability of various single-stranded RNAi agents and antisense oligonucleotides to mediate RNA interference or RNase H knockdown is described herein and shown, as non-limiting examples, in the Figures and Tables.

Experimental data (not shown) and described in detail elsewhere herein demonstrated that putative dual mechanism oligonucleotides are capable of mediating both RNA interference and RNase H knockdown. RNA interference was tested in either of two different in vitro Ago-2 assays, and RNase H knockdown was tested in an in vitro RNase H assay.

The experiments used an RNase H assay, with WV-1868 (ASO, mediating a RNase H knockdown mechanism) as a positive control, and WV-2110 (a single-stranded RNAi agent) as a negative control. RNA molecule WV-2372 is used as a test substrate. In the RNase H assay, dual mechanism oligonucleotide WV-2111 mediated RNase H knockdown.

Allele Specific Suppression

In some embodiments, provided oligonucleotides are capable of directing a decrease in the expression and/or level of a target gene or its gene product. In some embodiments, provided oligonucleotides are capable of mediating allele-specific suppression (or allele-specific knockdown).

In some embodiments, in some disease states, a patient (e.g., a human patient) can comprise two copies of the same gene, wherein one copy is wild-type (which is not disease-related), whereas the other copy on another chromosome has a mutation (which is disease-related). In some embodiments, the wild-type and mutant alleles can be differentiated by a particular sequence at the mutation, or else can be differentiated by a sequence outside the deleterious mutation (e.g., at a SNP). Knocking down both the mutant and wild-type alleles can sometimes be undesirable, because expression of the wild-type gene may be necessary or beneficial, while expression of the mutant gene may be deleterious or disease-related.

Without wishing to be bound by any particular theory, the present disclosure notes that, in many cases, introduction of a stereocontrolled chiral internucleotidic linkage (in place of a stereorandom chiral internucleotidic linkage) can increase the allele-specific suppression, stability, efficacy, specificity, delivery, and/or albumin binding of an APOC3 oligonucleotide.

In some embodiments, an APOC3 oligonucleotide capable of mediating allele-specific suppression comprises one or more stereopure chiral internucleotidic linkages.

In some embodiments, an APOC3 oligonucleotide capable of mediating allele-specific suppression comprises one or more stereopure chiral internucleotidic linkages in the Sp configuration.

In some embodiments, an APOC3 oligonucleotide capable of mediating allele-specific suppression comprises one or more stereopure chiral internucleotidic linkages in the Sp configuration and one or more stereopure chiral internucleotidic linkages in the Rp configuration.

In some embodiments, an APOC3 oligonucleotide capable of mediating allele-specific suppression comprises one or more stereopure phosphorothioates.

In some embodiments, an APOC3 oligonucleotide capable of mediating allele-specific suppression comprises one or more stereopure phosphorothioates in the Sp configuration.

In some embodiments, an APOC3 oligonucleotide capable of mediating allele-specific suppression comprises one or more stereopure phosphorothioates in the Rp configuration.

In some embodiments, an APOC3 oligonucleotide capable of mediating allele-specific suppression comprises one or more stereopure phosphorothioates in the Sp configuration and one or more stereopure phosphorothioates in the Rp configuration.

In some embodiments, an APOC3 oligonucleotide capable of allele-specific suppression of a target gene or its gene product can comprise any structure or format described herein.

Multimers of oligonucleotides

In some embodiments, a multimer comprises two or more of: an APOC3 oligonucleotide, an APOC3 oligonucleotide that directs RNA interference, an APOC3 oligonucleotide that directs RNase H-mediated knockdown and/or or an APOC3 oligonucleotide that directs both RNA interference and RNase H-mediated knockdown can have any format or structural element thereof described herein or known in the art.

In some embodiments, a provided composition comprises a combination of one or more provided oligonucleotide types. One of skill in the chemical and medicinal arts will recognize that the selection and amount of each of the one or more types of provided oligonucleotides in a provided composition will depend on the intended use of that composition. That is to say, one of skill in the relevant arts would design a provided chirally controlled oligonucleotide composition such that the amounts and types of provided oligonucleotides contained therein cause the composition as a whole to have certain desirable characteristics (e.g., biologically desirable, therapeutically desirable, etc.).

In some embodiments, a provided oligonucleotide type is selected from those described in WO/2014/012081 and WO/2015/107425, the oligonucleotides, oligonucleotide types, oligonucleotide compositions, and methods thereof of each of which are incorporated herein by reference. In some embodiments, a provided chirally controlled oligonucleotide composition comprises oligonucleotides of an APOC3 oligonucleotide type selected from those described in WO/2014/012081 and WO/2015/107425.

In some embodiments, the present disclosure pertains to a composition comprising a chirally controlled oligonucleotide composition, wherein the sequence of the oligonucleotide comprises or consists of the sequence of any chirally controlled oligonucleotide composition disclosed herein.

In some embodiments, the present disclosure pertains to a composition comprising a chirally controlled oligonucleotide composition, wherein the sequence of the oligonucleotide comprises or consists of the sequence of any single-stranded RNAi agent composition listed in Table 1A or otherwise described herein.

In some embodiments, the present disclosure pertains to compositions comprising a multimer of oligonucleotides, e.g., single-stranded RNAi agents, antisense oligonucleotides and/or other oligonucleotides, at least one of which has a structure, sequence or other characteristic as described herein.

In some embodiments, the present disclosure pertains to compositions comprising a multimer of single-stranded RNAi agents, antisense oligonucleotides and/or other oligonucleotides, wherein the multimer is at least about 16 kD in size.

In some embodiments, the present disclosure pertains to compositions comprising a multimer of single-stranded RNAi agents, antisense oligonucleotides and/or other oligonucleotides, and further comprises a carbohydrate moiety, lipid moiety, targeting moiety, or other compound.

In some embodiments, the present disclosure pertains to compositions comprising a multimer of single-stranded RNAi agents, antisense oligonucleotides and/or other oligonucleotides, and further comprises a carbohydrate moiety, lipid moiety, targeting moiety, or other compound, the total weight of which is at least about 16 kD in size.

In some embodiments, the multimer can comprise at least 2 single-stranded RNAi agents, antisense oligonucleotides and/or other oligonucleotides. In some embodiments, the multimer can comprise at least 3 single-stranded RNAi agents, antisense oligonucleotides and/or other oligonucleotides. In some embodiments, the multimer can comprise at least 4 single-stranded RNAi agents, antisense oligonucleotides and/or other oligonucleotides. In some embodiments, the multimer can comprise at least 5 single-stranded RNAi agents, antisense oligonucleotides and/or other oligonucleotides. In some embodiments, the multimer can comprise at least 6 single-stranded RNAi agents, antisense oligonucleotides and/or other oligonucleotides. In some embodiments, the multimer can comprise at least 7 single-stranded RNAi agents, antisense oligonucleotides and/or other oligonucleotides. In some embodiments, the multimer can comprise at least 8 single-stranded RNAi agents, antisense oligonucleotides and/or other oligonucleotides. In some embodiments, the multimer can comprise at least 9 single-stranded RNAi agents, antisense oligonucleotides and/or other oligonucleotides. In some embodiments, the multimer can comprise at least 10 single-stranded RNAi agents, antisense oligonucleotides and/or other oligonucleotides.

Without wishing to be bound by any particular theory, the present disclosure suggests that multimerization of oligonucleotides can provide a multimer which has a total molecular weight sufficient for transport via the lymphatic system. Supersaxo et al. reported that there is a linear relationship between the molecular weight of a drug and the proportion of a dose absorbed lymphatically, and that molecules with a molecular weight greater than 16 kD are absorbed mainly by the lymphatics, which drain a subcutaneous injection site. Supersaxo et al. 1990 Pharm. Res. 7: 167-9. In some embodiments, an APOC3 oligonucleotide has a molecular weight of around 8 kD. In some embodiments, a multimer comprising multiple oligonucleotides has a molecular weight of at least around 16 kD.

Without wishing to be bound by any particular theory, the present disclosure notes that subcutaneous injections are reportedly widely utilized for delivery of drugs, including, but not limited to, those with limited oral availability, or as a means to modify or extend the release profile. McLennan et al. 2005 Drug Disc. Today: Technologies 2: 89-96. Subcutaneous injection reportedly results in delivery to the interstitial area underlying the dermis of the skin, from where drugs enter the circulatory system, or the lymphatic system; transport is reportedly affected by molecular weight, particle size, charge, hydrophilicity, and interaction with components in the interstitium. Drug formulation characteristics, such as drug concentration, injection volume, ionic strength, viscosity, and pH can also all reportedly play roles in diffusion from the subcutaneous injection site. McLennan et al. 2005; Paniagua et al. 2012 Lymphology 45: 144-153; and Bagby et al. 2012 Pharmaceutics 4: 276-295.

In some embodiments, one or more characteristic of molecular weight, particle size, charge, hydrophilicity, and interaction with components in the interstitium, drug concentration, injection volume, ionic strength, viscosity, and/or pH are modulated to improve or maximize the efficacy, bioavailability or delivery of a composition comprising an APOC3 oligonucleotide.

As noted above, molecules with a molecular weight greater than 16 kD are reportedly absorbed mainly by the lymphatics. Supersaxo et al. 1990 Pharm. Res. 7: 167-9. In some embodiments, the present disclosure pertains to a composition comprising a multimer of oligonucleotides, wherein the multimer has a total molecular weight of at least about 16 kD. In some embodiments, the present disclosure pertains to a composition comprising two or more different types or sizes of multimers of oligonucleotides, wherein the one or more of the different types of multimer has a total molecular weight of at least about 16 kD.

In some embodiments, each oligonucleotide in a multimer can target the same or different targets. In some embodiments, wherein the each oligonucleotide in a multimer can target the same or different targets, administration of the multimer can be used to treat a disease involving over-expression or multiple target genes. In some embodiments, wherein the each oligonucleotide in a multimer can target the same or different targets, administration of the multimer can be used to treat different diseases involving over-expression of different target genes.

In some embodiments, each oligonucleotide in a multimer can target the same sequence in the same target. In some embodiments, each oligonucleotide in a multimer can target different sequences in the same target.

Non-limiting examples of multimers are shown in Table 89A.

In some embodiments, a multimer comprises two or more oligonucleotides directly connected to each other (e.g., via a bond or direct bond, such as a covalent bond), or via a linker.

Any linker described herein or known in the art can be used to link the oligonucleotides in a multimer. Various approaches for construction of multimers and use of various linkers is illustrated in Table 89B and 89C.

Without wishing to be bound by any particular theory, the present disclosure notes that, in at least some cases, a phosphorothioate in the Rp configuration is particularly susceptible to nuclease cleavage. In some embodiments of Multimer Type 2, a multimer is essentially a single long oligonucleotide, wherein the oligonucleotide comprises multiple shorter oligonucleotides, which are connected by short linker oligonucleotides. In some embodiments of Multimer Type 2, a multimer is essentially a single long oligonucleotide, wherein the oligonucleotide comprises multiple shorter oligonucleotides, which are connected by short linker oligonucleotides, wherein the short linker oligonucleotides comprise one or more internucleotidic linkages in the Rp configuration. In some embodiments of Multimer Type 2, a multimer is essentially a single long oligonucleotide, wherein the oligonucleotide comprises multiple shorter oligonucleotides, which are connected by short linker oligonucleotides, wherein the short linker oligonucleotides comprise one or more phosphorothioates in the Rp configuration.

Non-limiting examples of linkers include: a cleavable linker or a biodegradable linker; a non-cleavable or non-biodegradable linker; a linker comprising one or more internucleotidic linkages comprising a chiral center in the Rp configuration; a linker comprising one or more internucleotidic linkages comprising a chiral phosphorus in the Rp configuration; a linker comprising one or more phosphorothioate in the Rp configuration; a linker comprising two or more phosphorothioate in the Rp configuration; a linker comprising three or more phosphorothioate in the Rp configuration; a photocleavable linker; 1-(5-(N-maleimidomethyl)-2-nitrophenyl)ethanol N-hydroxysuccinimide ester; a linker comprising a maleimido moiety; a linker comprising a N-hydroxysuccinimide ester moiety; a linker conjugated to an APOC3 oligonucleotide at a base; a linker conjugated to an APOC3 oligonucleotide at an internucleotidic linkage; a linker conjugated at a sugar; a phosphodiester; a phosphotriester; a methylphosphonate; a P3′→N5′ phosphoramidate; a N3′→P5′ phosphoramidate; a N3′→P5′ thio-phosphoramidate; a phosphorothioate linkage; a thiourea linker; a C5 or C6 linker, as described in U.S. Pat. No. 9,572,891; a linker comprising a alkyl, cycloalkyl, aryl, heterocyclyl, and heteroaryl; a linker comprising a substituted alkyl, cycloalkyl, aryl, heterocyclyl, and heteroaryl; a linker of the structure of formula (A) of U.S. Pat. No. 9,512,163; a linker comprising a C1-C12 hydrocarbyl chain; a polyethylene glycol linker; a hexaethylene glycol linker; a hydrocarbyl chain; a substituted hydrocarbyl chain; a linker comprising one or more of: alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, or alkynylhereroaryl; a linker comprising a peptide having an amino acid sequence selected from: ALAL, APISFFELG, FL, GFN, R/KXX, GRWHTVGLRWE, YL, GF, and FF, in which X is any amino acid; a linker comprising the formula —(CH2)wS-S(CH2)m-, wherein n and m are independently integers from 0 to 10; a linker comprising a low pH-labile bond; a linker comprising a low pH-labile bond comprising an amine, an imine, an ester, a benzoic imine, an amino ester, a diortho ester, a polyphosphoester, a polyphosphazene, an acetal, a vinyl ether, a hydrazone, an azidomethyl-methylmaleic anhydride, a thiopropionate, a masked endosomolytic agent or a citraconyl group; a branched linker; a cleavable linker susceptible to cleavage agents, e.g., pH, redox potential or the presence of degradative molecules; a redox cleavable linker; a phosphate-based cleavable linker; a phosphate-based cleavable linker comprising: —O—P(O)(OH)—O—, —O—P(S)(OH)—O—, —O—P(S)(SH—O—, —O—P(O)(OH—S—, —S—P(O)(OH—S—, —O—P(S)(OH—S—, —S—P(S)(OH—O—, —O—P(O)(H)—O—, —O—P(S)(H)—O—, —S—P(O)(H)—O—, —S—P(S)(H)—O—, —S—P(O)(H—S—, or —O—P(S)(H—S—; an acid cleavable linker; an ester-based linker; a peptide-based cleavable linker; and moieties comprising any of these linkers.

In some embodiments, a linker comprises a polypeptide that is more susceptible to cleavage by an endopeptidase in the mammalian extract than the targeting oligonucleotides. In some embodiments, the endopeptidase is trypsin, chymotrypsin, elastase, thermolysin, pepsin, or endopeptidase V8. In some embodiments, the endopeptidase is cathepsin B, cathepsin D, cathepsin L, cathepsin C, papain, cathepsin S or endosomal acidic insulinase.

Various linkers and methods of multimerization of oligonucleotides are described in, as non-limiting examples: U.S. Pat. Nos. 9,370,582; 9,371,348; 9,512,163; 9,572,891; and 6,031,091; and international published patent applications WO1998000435; WO2014043544; and WO2013040429.

The disclosure also notes that any linker described herein, or known in the art, can be used to link one or more oligonucleotides to each other, or to link one or more moiety (as non-limiting examples, a targeting moiety, a carbohydrate moiety, a GalNAc moiety, a lipid moiety, etc.) to one or more oligonucleotides (as non-limiting examples, a single-stranded RNAi agent, an antisense oligonucleotide, a double-stranded RNAi agent, an APOC3 oligonucleotide capable of directing or inhibiting exon skipping, etc.).

### Example Methods for Preparing Oligonucleotides and Compositions

Methods for preparing provided oligonucleotides and oligonucleotide compositions are widely known in the art, including but not limited to those described in WO/2010/064146, WO/2011/005761, WO/2013/012758, WO/2014/010250, US2013/0178612, WO/2014/012081, WO/2015/107425, PCT/US2016/043542, and PCT/US2016/043598, the methods and reagents of each of which is incorporated herein by reference.

Chirally Controlled Oligonucleotides.

In some embodiments, provided oligonucleotides are capable of directing a decrease in the expression and/or level of a target gene or its gene product. In some embodiments, provided oligonucleotides are chirally controlled.

In some embodiments, the present disclosure provides chirally controlled oligonucleotide compositions that, when compared to a reference condition [e.g., absence of the composition, presence of a reference composition (e.g., a stereorandom composition of oligonucleotides having the same base sequence, the same chemical modifications, etc., an APOC3 oligonucleotide or single-stranded RNAi agent of another stereoisomer, etc.), and combinations thereof], are capable of directing a decrease in the expression and/or level of a target gene or its gene product.

In some embodiments, the present disclosure provides chirally controlled oligonucleotide compositions that, when compared to a reference condition [e.g., absence of the composition, presence of a reference composition (e.g., a stereorandom composition of oligonucleotides having the same base sequence, the same chemical modifications, etc., an APOC3 oligonucleotide or single-stranded RNAi agent of another stereoisomer, etc.), and combinations thereof], mediate improved knockdown of transcripts via single-stranded RNA interference or RNase H.

Among other things, the present disclosure provides chirally controlled ssRNAi agents and chirally controlled compositions comprising one or more specific nucleotide types. In some embodiments, the phrase “oligonucleotide type,” as used herein, defines an APOC3 oligonucleotide that has a particular base sequence, pattern of backbone linkages, pattern of backbone chiral centers, and pattern of backbone phosphorus modifications (e.g., “—XLR¹” groups). Oligonucleotides of a common designated “type” are structurally identical to one another with respect to base sequence, pattern of backbone linkages, pattern of backbone chiral centers, and pattern of backbone phosphorus modifications. In some embodiments, oligonucleotides of an APOC3 oligonucleotide type are identical.

In some embodiments, a provided chirally controlled oligonucleotide or single-stranded RNAi agent in the disclosure has properties different from those of the corresponding stereorandom oligonucleotide or single-stranded RNAi agent mixture. In some embodiments, a chirally controlled oligonucleotide or single-stranded RNAi agent has lipophilicity different from that of the stereorandom oligonucleotide or single-stranded RNAi agent mixture. In some embodiments, a chirally controlled oligonucleotide or single-stranded RNAi agent has different retention time on HPLC. In some embodiments, a chirally controlled oligonucleotide or single-stranded RNAi agent may have a peak retention time significantly different from that of the corresponding stereorandom oligonucleotide or single-stranded RNAi agent mixture. During oligonucleotide or single-stranded RNAi agent purification using HPLC as generally practiced in the art, certain chirally controlled oligonucleotide or single-stranded RNAi agents will be largely if not totally lost. During oligonucleotide or single-stranded RNAi agent purification using HPLC as generally practiced in the art, certain chirally controlled oligonucleotide or single-stranded RNAi agents will be largely if not totally lost. One of the consequences is that certain diastereomers of a stereorandom oligonucleotide or single-stranded RNAi agent mixture (certain chirally controlled oligonucleotide or single-stranded RNAi agents) are not tested in assays. Another consequence is that from batches to batches, due to the inevitable instrumental and human errors, the supposedly “pure” stereorandom oligonucleotide or single-stranded RNAi agent will have inconsistent compositions in that diastereomers in the composition, and their relative and absolute amounts, are different from batches to batches. The chirally controlled oligonucleotide or single-stranded RNAi agent and chirally controlled oligonucleotide or single-stranded RNAi agent compositions provided in this disclosure overcome such problems, as a chirally controlled oligonucleotide or single-stranded RNAi agent is synthesized in a chirally controlled fashion as a single diastereomer (diastereoisomer), and a oligonucleotide or single-stranded RNAi agent comprises predetermined levels of one or more individual oligonucleotide or single-stranded RNAi agent types.

One of skill in the chemical and synthetic arts will appreciate that synthetic methods of the present disclosure provide for a degree of control during each step of the synthesis of a provided single-stranded RNAi agent such that each nucleotide unit of the single-stranded RNAi agent can be designed and/or selected in advance to have a particular stereochemistry at the linkage phosphorus and/or a particular modification at the linkage phosphorus, and/or a particular base, and/or a particular sugar. In some embodiments, a provided single-stranded RNAi agent is designed and/or selected in advance to have a particular combination of stereocenters at the linkage phosphorus of the internucleotidic linkage.

In some embodiments, a provided single-stranded RNAi agent made using methods of the present disclosure is designed and/or determined to have a particular combination of linkage phosphorus modifications. In some embodiments, a provided single-stranded RNAi agent made using methods of the present disclosure is designed and/or determined to have a particular combination of bases. In some embodiments, a provided single-stranded RNAi agent made using methods of the present disclosure is designed and/or determined to have a particular combination of sugars. In some embodiments, a provided single-stranded RNAi agent made using methods of the present disclosure is designed and/or determined to have a particular combination of one or more of the above structural characteristics.

Methods of the present disclosure exhibit a high degree of chiral control. For instance, methods of the present disclosure facilitate control of the stereochemical configuration of every single linkage phosphorus within a provided single-stranded RNAi agent. In some embodiments, methods of the present disclosure provide an single-stranded RNAi agent comprising one or more modified internucleotidic linkages independently having the structure of Formula I.

In some embodiments, methods of the present disclosure provide an single-stranded RNAi agent which is a unimer. In some embodiments, methods of the present disclosure provide an single-stranded RNAi agent which is a unimer of configuration Rp. In some embodiments, methods of the present disclosure provide an single-stranded RNAi agent which is a unimer of configuration Sp.

In some embodiments, methods of the present disclosure provide a chirally controlled single-stranded RNAi agent composition, i.e., an single-stranded RNAi agent composition that contains predetermined levels of individual single-stranded RNAi agent types. In some embodiments an APOC3 oligonucleotide or a single-stranded RNAi agent comprises one single-stranded RNAi agent type. In some embodiments, a single-stranded RNAi agent comprises more than one single-stranded RNAi agent type. In some embodiments, an APOC3 oligonucleotide or single-stranded RNAi agent composition comprises a plurality of oligonucleotide and/or single-stranded RNAi agent types. Example chirally controlled oligonucleotide and single-stranded RNAi agent compositions made in accordance with the present disclosure are described herein.

In some embodiments, an APOC3 oligonucleotide comprises a chiral internucleotidic linkage (e.g., is stereocontrolled). In some embodiments, an APOC3 oligonucleotide comprises a chiral internucleotidic linkage which is stereocontrolled and a chiral internucleotidic linkage which is not stereocontrolled. In some embodiments, an APOC3 oligonucleotide comprises a chiral internucleotidic linkage which is stereocontrolled and an internucleotidic linkage which is not chiral. Various non-limiting examples of formats of stereocontrolled (chirally controlled) oligonucleotides are shown in Tables 71A to 71C. In some embodiments, an APOC3 oligonucleotide has a structure of Format 51. In some embodiments, an APOC3 oligonucleotide has a structure of Format S2. In some embodiments, an APOC3 oligonucleotide has a structure of Format S3. In some embodiments, an APOC3 oligonucleotide has a structure of Format S4. In some embodiments, an APOC3 oligonucleotide has a structure of Format S5. In some embodiments, an APOC3 oligonucleotide has a structure of Format S6. In some embodiments, an APOC3 oligonucleotide has a structure of Format S7. In some embodiments, an APOC3 oligonucleotide has a structure of Format S8. In some embodiments, an APOC3 oligonucleotide has a structure of Format S9. In some embodiments, an APOC3 oligonucleotide has a structure of Format S10. In some embodiments, an APOC3 oligonucleotide has a structure of Format S11. In some embodiments, an APOC3 oligonucleotide has a structure of Format S12. In some embodiments, an APOC3 oligonucleotide has a structure of Format S13. In some embodiments, an APOC3 oligonucleotide has a structure of Format S14. In some embodiments, an APOC3 oligonucleotide has a structure of Format S15. In some embodiments, an APOC3 oligonucleotide has a structure of Format S16. In some embodiments, an APOC3 oligonucleotide has a structure of Format S17. In some embodiments, an APOC3 oligonucleotide has a structure of Format S18. In some embodiments, an APOC3 oligonucleotide has a structure of Format S19. In some embodiments, an APOC3 oligonucleotide has a structure of Format S20. In some embodiments, an APOC3 oligonucleotide has a structure of Format S21. In some embodiments, an APOC3 oligonucleotide has a structure of Format S22. In some embodiments, an APOC3 oligonucleotide has a structure of Format S23. In some embodiments, an APOC3 oligonucleotide has a structure of Format S24. In some embodiments, an APOC3 oligonucleotide has a structure of Format S25. In some embodiments, an APOC3 oligonucleotide has a structure of Format S26. In some embodiments, an APOC3 oligonucleotide has a structure of Format S27. In some embodiments, an APOC3 oligonucleotide has a structure of Format S28. In some embodiments, an APOC3 oligonucleotide has a structure of Format S29. In some embodiments, an APOC3 oligonucleotide has a structure of Format S30. In some embodiments, an APOC3 oligonucleotide has a structure of Format S31. In some embodiments, an APOC3 oligonucleotide has a structure of Format S32. In some embodiments, an APOC3 oligonucleotide has a structure of Format S33. In some embodiments, an APOC3 oligonucleotide has a structure of Format S34. In some embodiments, an APOC3 oligonucleotide has a structure of Format S35. In some embodiments, an APOC3 oligonucleotide has a structure of Format S36. In some embodiments, an APOC3 oligonucleotide has a structure of Format S37. In some embodiments, an APOC3 oligonucleotide has a structure of Format S38. In some embodiments, an APOC3 oligonucleotide has a structure of Format S39. In some embodiments, an APOC3 oligonucleotide has a structure of Format S40. In some embodiments, an APOC3 oligonucleotide has a structure of Format S41. In some embodiments, an APOC3 oligonucleotide has a structure of Format S42. In some embodiments, an APOC3 oligonucleotide has a structure of Format S43. In some embodiments, an APOC3 oligonucleotide has a structure of Format S44.

In some embodiments, methods of the present disclosure provide chirally pure compositions with respect to the configuration of the linkage phosphorus. That is to say, in some embodiments, methods of the present disclosure provide compositions of wherein the oligonucleotide exists in the composition in the form of a single diastereomer with respect to the configuration of the linkage phosphorus.

In some embodiments, methods of the present disclosure provide chirally uniform compositions with respect to the configuration of the linkage phosphorus. That is to say, in some embodiments, methods of the present disclosure provide compositions of in which all nucleotide units therein have the same stereochemistry with respect to the configuration of the linkage phosphorus, e.g., all nucleotide units are of the Rp configuration at the linkage phosphorus or all nucleotide units are of the Sp configuration at the linkage phosphorus.

In some embodiments, an APOC3 oligonucleotide or a single-stranded RNAi agent comprises at least one Sp (e.g., a phosphorothioate or other internucleotidic linkage having a chiral center, in the Sp configuration). In some embodiments, an APOC3 oligonucleotide or a single-stranded RNAi agent comprises at least 5 Sp. In some embodiments, an APOC3 oligonucleotide or a single-stranded RNAi agent comprises at least 10 Sp. In some embodiments, an APOC3 oligonucleotide or a single-stranded RNAi agent comprises at least 15 to 25 Sp.

As shown herein, the incorporation of one or more Sp internucleotidic linkage or one or more Sp PS (phosphorothioate) performs two functions for a single-stranded RNAi agent: (a) it increases stability against nucleases; and (b) does not interfere with RNA interference activity. While single-stranded RNAi agents and double-stranded RNAi agents differ in many aspects, this disclosure notes that, reportedly, many chemical modifications have been attempted for double-stranded RNAi agents, wherein the modifications did not both (a) stabilize the molecule against nucleases; and (b) allow RNA interference activity. Many chemical modifications reportedly perform one function but not the other. Some chemical modifications of double-stranded RNAi agents reportedly stabilized the molecule against nucleases, but interfered with or abolished RNAi activity. Other chemical modifications of double-stranded RNAi agents reportedly did not interfere with RNAi activity, but also did not stabilize the molecules against nucleases. See, for example, Czauderna et al. 2003 Nucl. Acids Res. 31: 2705-2716; Hadwiger et al. 2005, pages 194-206, in RNA interference Technology, ed. K. Appasani, Cambridge University Press, Cambridge, UK; Deleavey et al. 2009 Curr. Prot. Nucl. Acid Chem. 16.3.1-16.3.22; Terrazas et al. 2009 Nucl. Acids Res. 37: 346-353; Schwarz et al. 2002 Mol. Cell 10: 537-548; and Lipardi et al. 2001 Cell 107: 299-307. Only a minority of chemical modifications of double-stranded RNAi agents were capable of performing both functions. Furthermore, Matranga et al. 2005 Cell 123: 607-620 showed that introduction of a single Sp internucleotidic linkage (e.g., a single Sp PS) into the sense strand of a double-stranded RNAi agent greatly decreased RISC assembly and RNA interference activity. Thus, the chemical modification of a double-stranded RNAi agent with a single Sp internucleotidic linkage (e.g., a single Sp PS) did not (b) allow RNA interference activity. Thus, this disclosure endeavored to test the effect(s) of incorporation of Sp internucleotidic linkages or Sp PS into a single-stranded RNAi agent. The data shown herein show that, surprisingly, the incorporation of a Sp internucleotidic linkage or Sp PS performs two functions for a single-stranded RNAi agent: (a) it increases stability against nucleases; and (b) does not interfere with RNA interference activity.

As shown in the data shown in Table 45, the stability of a single-stranded RNAi agent against nucleases was increased by converting a stereorandom phosphorothioate at the 5′-end and/or 3′-end to a phosphorothioate in the Sp configuration. Additional increases in stability were obtained by converting stereorandom phosphorothioates at nuclease cleavage sites identified herein to phosphorothioates in the Sp configuration.

Without wishing to be bound by any particular theory, the disclosure suggests that incorporation of phosphorothioates or other chiral internucleotidic linkages in a Sp configuration may protect single-stranded RNAi agents from nucleases. Table 45 indicates various nuclease cleavage sites identified in a stereorandom APOC3 single-stranded RNAi agent, WV-2817. These major cleavage sites are between two pyrimidines (5′-U′U-3′, 5′-U′U-3′ or 5′-T′U-3′, where indicates the cleavage site). Additional major nuclease cleavage sites were identified for stereorandom single-stranded RNAi agent WV-3242: 5′-U′U-3′, 5′—C′U-3′, and 5′-T′U-3′. Of the six major nuclease cleavage sites, five were between two adjacent pyrimidines and one was adjacent to a pyrimidine. Experimental data shown in Table 45 indicates that replacing one or more of the nuclease cleavage sites with a Sp internucleotidic linkage (or chiral internucleotidic linkage in a Sp configuration, e.g., a Sp PS or a phosphorothioate in the Sp configuration) greatly increased the stability of the single-stranded RNAi agents.

Single-stranded RNAi agents comprising multiple Sp internucleotidic linkages (e.g., Sp PS) were also tested to determine if the Sp abolished RNAi activity. The present disclosure notes that previous work has shown that many stereorandom oligonucleotides can decrease or completely lose their enzymatic or biological activity if converted into stereocontrolled versions. For many previously described oligonucleotides, introduction of Sp internucleotidic linkages can decrease or abolish activity.

Table 44 shows that, surprisingly, in addition to increasing stability, replacing multiple internucleotidic linkages (whether stereorandom or phosphodiester) with Sp internucleotidic linkages (e.g., Sp PS) did not decrease or eliminate RNA interference activity of a single-stranded RNAi agent. These results are also surprising because, reportedly, the introduction of a Sp PS into a stereorandom oligonucleotide in many cases is known to reduce biological activity. Thus, the introduction of one or more Sp internucleotidic linkages or Sp PS both increased stability of a single-stranded RNAi agent, and did not decrease or abolish RNAi activity.

Table 69A to C also shows that, surprisingly, in addition to increasing stability, replacing multiple internucleotidic linkages (whether stereorandom or phosphodiester) with Sp internucleotidic linkages (e.g., Sp PS) increased stability in and simultaneously did not decrease or eliminate RNA interference activity of a single-stranded RNAi agent. In some cases, activity was increased. These results are also surprising because, reportedly, the introduction of a Sp PS into a stereorandom oligonucleotide in many cases is known to reduce biological activity. Thus, the introduction of one or more Sp internucleotidic linkages or Sp PS both increased stability of a single-stranded RNAi agent, and did not decrease or abolish RNAi activity.

In some embodiments or a single-stranded RNAi agent, the oligonucleotide or single-stranded RNAi comprises 1 or more Sp internucleotidic linkages. In some embodiments or a single-stranded RNAi agent, the oligonucleotide or single-stranded RNAi comprises 1 or more Sp internucleotidic linkages at the 5′ and/or 3′-end of the oligonucleotide or single-stranded RNAi agent. In some embodiments or a single-stranded RNAi agent, the oligonucleotide or single-stranded RNAi comprises 1 or more Sp internucleotidic linkages at sites susceptible to nuclease cleavage.

In some embodiments or a single-stranded RNAi agent, the oligonucleotide or single-stranded RNAi comprises 1 or more Sp PS. In some embodiments or a single-stranded RNAi agent, the oligonucleotide or single-stranded RNAi comprises 1 or more Sp PS at the 5′ and/or 3′-end of the oligonucleotide or single-stranded RNAi agent. In some embodiments or a single-stranded RNAi agent, the oligonucleotide or single-stranded RNAi comprises 1 or more Sp PS at sites susceptible to nuclease cleavage.

Among other things, the present disclosure recognizes the challenge of stereoselective (rather than stereorandom or racemic) preparation of single-stranded RNAi agents. Among other things, the present disclosure provides methods and reagents for stereoselective preparation of single-stranded RNAi agents comprising multiple (e.g., more than 5, 6, 7, 8, 9, or 10) internucleotidic linkages, and particularly for single-stranded RNAi agents comprising multiple (e.g., more than 5, 6, 7, 8, 9, or 10) chiral internucleotidic linkages. In some embodiments, in a stereorandom or racemic preparation of single-stranded RNAi agents, at least one chiral internucleotidic linkage is formed with less than 90:10, 95:5, 96:4, 97:3, or 98:2 diastereoselectivity. In some embodiments, for a stereoselective or chirally controlled preparation of single-stranded RNAi agents, each chiral internucleotidic linkage is formed with greater than 90:10, 95:5, 96:4, 97:3, or 98:2 diastereoselectivity. In some embodiments, for a stereoselective or chirally controlled preparation of single-stranded RNAi agents, each chiral internucleotidic linkage is formed with greater than 95:5 diastereoselectivity. In some embodiments, for a stereoselective or chirally controlled preparation of single-stranded RNAi agents, each chiral internucleotidic linkage is formed with greater than 96:4 diastereoselectivity. In some embodiments, for a stereoselective or chirally controlled preparation of single-stranded RNAi agents, each chiral internucleotidic linkage is formed with greater than 97:3 diastereoselectivity. In some embodiments, for a stereoselective or chirally controlled preparation of single-stranded RNAi agents, each chiral internucleotidic linkage is formed with greater than 98:2 diastereoselectivity. In some embodiments, for a stereoselective or chirally controlled preparation of single-stranded RNAi agents, each chiral internucleotidic linkage is formed with greater than 99:1 diastereoselectivity. In some embodiments, diastereoselectivity of a chiral internucleotidic linkage in an single-stranded RNAi agent may be measured through a model reaction, e.g. formation of a dimer under essentially the same or comparable conditions wherein the dimer has the same internucleotidic linkage as the chiral internucleotidic linkage, the 5′-nucleoside of the dimer is the same as the nucleoside to the 5′-end of the chiral internucleotidic linkage, and the 3′-nucleoside of the dimer is the same as the nucleoside to the 3′-end of the chiral internucleotidic linkage.

In some embodiments, an APOC3 oligonucleotide or a single-stranded RNAi agent is a composition designed to comprise multiple oligonucleotide or single-stranded RNAi agent types. In some embodiments, methods of the present disclosure allow for the generation of a library of chirally controlled single-stranded RNAi agents such that a pre-selected amount of any one or more chirally controlled single-stranded RNAi agent types can be mixed with any one or more other chirally controlled single-stranded RNAi agent types to create a chirally controlled single-stranded RNAi agent composition. In some embodiments, the pre-selected amount of an single-stranded RNAi agent type is a composition having any one of the above-described diastereomeric purities.

In some embodiments, the present disclosure provides methods for making a chirally controlled single-stranded RNAi agent comprising steps of:

(1) coupling;

(2) capping;

(3) modifying;

(4) deblocking; and

(5) repeating steps (1)-(4) until a desired length is achieved.

When describing the provided methods, the word “cycle” has its ordinary meaning as understood by a person of ordinary skill in the art. In some embodiments, one round of steps (1)-(4) is referred to as a cycle.

In some embodiments, the present disclosure provides methods for making chirally controlled single-stranded RNAi agent compositions, comprising steps of:

(a) providing an amount of a first chirally controlled single-stranded RNAi agent; and

(b) optionally providing an amount of one or more additional chirally controlled single-stranded RNAi agents.

In some embodiments, a first chirally controlled single-stranded RNAi agent is an single-stranded RNAi agent type, as described herein. In some embodiments, a one or more additional chirally controlled single-stranded RNAi agent is a one or more single-stranded RNAi agent type, as described herein.

One of skill in the relevant chemical and synthetic arts will recognize the degree of versatility and control over structural variation and stereochemical configuration of a provided single-stranded RNAi agent when synthesized using methods of the present disclosure. For instance, after a first cycle is complete, a subsequent cycle can be performed using a nucleotide unit individually selected for that subsequent cycle which, in some embodiments, comprises a nucleobase and/or a sugar that is different from the first cycle nucleobase and/or sugar. Likewise, the chiral auxiliary used in the coupling step of the subsequent cycle can be different from the chiral auxiliary used in the first cycle, such that the second cycle generates a phosphorus linkage of a different stereochemical configuration. In some embodiments, the stereochemistry of the linkage phosphorus in the newly formed internucleotidic linkage is controlled by using stereochemically pure phosphoramidites. Additionally, the modification reagent used in the modifying step of a subsequent cycle can be different from the modification reagent used in the first or former cycle. The cumulative effect of this iterative assembly approach is such that each component of a provided single-stranded RNAi agent can be structurally and configurationally tailored to a high degree. An additional advantage to this approach is that the step of capping minimizes the formation of “n−1” impurities that would otherwise make isolation of a provided single-stranded RNAi agent extremely challenging, and especially single-stranded RNAi agents of longer lengths.

In some embodiments, an example cycle of the method for making chirally controlled single-stranded RNAi agents is illustrated in example schemes described in the present disclosure. In some embodiments, an example cycle of the method for making chirally controlled single-stranded RNAi agents is illustrated in Scheme I. In some embodiments, [symbol] represents the solid support, and optionally a portion of the growing chirally controlled single-stranded RNAi agent attached to the solid support. The chiral auxiliary exemplified has the structure of formula 3-I:

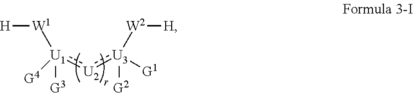

which is further described below. “Cap” is any chemical moiety introduced to the nitrogen atom by the capping step, and in some embodiments, is an amino protecting group. One of ordinary skill in the art understands that in the first cycle, there may be only one nucleoside attached to the solid support when started, and cycle exit can be performed optionally before deblocking. As understood by a person of skill in the art, B^{PRO} is a protected base used in single-stranded RNAi agent synthesis. Each step of the above-depicted cycle of Scheme I is described further below.

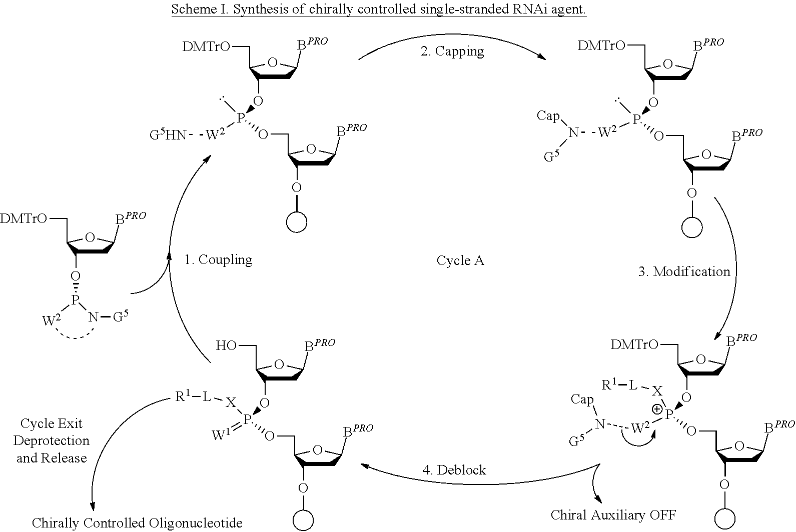

### Synthesis on Solid Support

In some embodiments, the synthesis of a provided single-stranded RNAi agent is performed on solid phase. In some embodiments, reactive groups present on a solid support are protected. In some embodiments, reactive groups present on a solid support are unprotected. During single-stranded RNAi agent synthesis a solid support is treated with various reagents in several synthesis cycles to achieve the stepwise elongation of a growing single-stranded RNAi agent chain with individual nucleotide units. The nucleoside unit at the end of the chain which is directly linked to the solid support is termed “the first nucleoside” as used herein. A first nucleoside is bound to a solid support via a linker moiety, i.e. a diradical with covalent bonds between either of a CPG, a polymer or other solid support and a nucleoside. The linker stays intact during the synthesis cycles performed to assemble the oligonucleotide chain and is cleaved after the chain assembly to liberate the oligonucleotide from the support.

Solid supports for solid-phase nucleic acid synthesis include the supports described in, e.g., U.S. Pat. Nos. 4,659,774, 5,141,813, 4,458,066; Caruthers U.S. Pat. Nos. 4,415,732, 4,458,066, 4,500,707, 4,668,777, 4,973,679, and 5,132,418; Andrus et al. U.S. Pat. Nos. 5,047,524, 5,262,530; and Koster U.S. Pat. No. 4,725,677 (reissued as RE34,069). In some embodiments, a solid phase is an organic polymer support. In some embodiments, a solid phase is an inorganic polymer support. In some embodiments, an organic polymer support is polystyrene, aminomethyl polystyrene, a polyethylene glycol-polystyrene graft copolymer, polyacrylamide, polymethacrylate, polyvinylalcohol, highly cross-linked polymer (HCP), or other synthetic polymers, carbohydrates such as cellulose and starch or other polymeric carbohydrates, or other organic polymers and any copolymers, composite materials or combination of the above inorganic or organic materials. In some embodiments, an inorganic polymer support is silica, alumina, controlled polyglass (CPG), which is a silica-gel support, or aminopropyl CPG. Other useful solid supports include fluorous solid supports (see e.g., WO/2005/070859), long chain alkylamine (LCAA) controlled pore glass (CPG) solid supports (see e.g., S. P. Adams, K. S. Kavka, E. J. Wykes, S. B. Holder and G. R. Galluppi, J. Am. Chem. Soc., 1983, 105, 661-663; G. R. Gough, M. J. Bruden and P. T. Gilham, Tetrahedron Lett., 1981, 22, 4177-4180). Membrane supports and polymeric membranes (see e.g. Innovation and Perspectives in Solid Phase Synthesis, Peptides, Proteins and Nucleic Acids, ch 21 pp 157-162, 1994, Ed. Roger Epton and U.S. Pat. No. 4,923,901) are also useful for the synthesis of nucleic acids. Once formed, a membrane can be chemically functionalized for use in nucleic acid synthesis. In addition to the attachment of a functional group to the membrane, the use of a linker or spacer group attached to the membrane is also used in some embodiments to minimize steric hindrance between the membrane and the synthesized chain.

Other suitable solid supports include those generally known in the art to be suitable for use in solid phase methodologies, including, for example, glass sold as Primer™ 200 support, controlled pore glass (CPG), oxalyl-controlled pore glass (see, e.g., Alul, et al., Nucleic Acids Research, 1991, 19, 1527), TentaGel Support-an aminopolyethyleneglycol derivatized support (see, e.g., Wright, et al., Tetrahedron Lett., 1993, 34, 3373), and Poros-a copolymer of polystyrene/divinylbenzene.

Surface activated polymers have been demonstrated for use in synthesis of natural and modified nucleic acids and proteins on several solid supports mediums. A solid support material can be any polymer suitably uniform in porosity, having sufficient amine content, and sufficient flexibility to undergo any attendant manipulations without losing integrity. Examples of suitable selected materials include nylon, polypropylene, polyester, polytetrafluoroethylene, polystyrene, polycarbonate, and nitrocellulose. Other materials can serve as a solid support, depending on the design of the investigator. In consideration of some designs, for example, a coated metal, in particular gold or platinum can be selected (see e.g., US publication No. 20010055761). In one embodiment of single-stranded RNAi agent synthesis, for example, a nucleoside is anchored to a solid support which is functionalized with hydroxyl or amino residues. Alternatively, a solid support is derivatized to provide an acid labile trialkoxytrityl group, such as a trimethoxytrityl group (TMT). Without being bound by theory, it is expected that the presence of a trialkoxytrityl protecting group will permit initial detritylation under conditions commonly used on DNA synthesizers. For a faster release of single-stranded RNAi agent material in solution with aqueous ammonia, a diglycoate linker is optionally introduced onto the support.

In some embodiments, a provided single-stranded RNAi agent alternatively is synthesized from the 5′ to 3′ direction. In some embodiments, a nucleic acid is attached to a solid support through its 5′ end of the growing nucleic acid, thereby presenting its 3′ group for reaction, i.e. using 5′-nucleoside phosphoramidites or in enzymatic reaction (e.g. ligation and polymerization using nucleoside 5′-triphosphates). When considering the 5′ to 3′ synthesis the iterative steps of the present disclosure remain unchanged (i.e. capping and modification on the chiral phosphorus).

### Linking Moiety

A linking moiety or linker is optionally used to connect a solid support to a compound comprising a free nucleophilic moiety. Suitable linkers are known such as short molecules which serve to connect a solid support to functional groups (e.g., hydroxyl groups) of initial nucleosides molecules in solid phase synthetic techniques. In some embodiments, the linking moiety is a succinamic acid linker, or a succinate linker (—CO—CH₂—CH₂—CO—), or an oxalyl linker (—CO—CO—). In some embodiments, the linking moiety and the nucleoside are bonded together through an ester bond. In some embodiments, a linking moiety and a nucleoside are bonded together through an amide bond. In some embodiments, a linking moiety connects a nucleoside to another nucleotide or nucleic acid. Suitable linkers are disclosed in, for example, Oligonucleotides And Analogues A Practical Approach, Ekstein, F. Ed., IRL Press, N.Y., 1991, Chapter 1 and Solid-Phase Supports for Oligonucleotide Synthesis, Pon, R. T., Curr. Prot. Nucleic Acid Chem., 2000, 3.1.1-3.1.28.

A linker moiety is used to connect a compound comprising a free nucleophilic moiety to another nucleoside, nucleotide, or nucleic acid. In some embodiments, a linking moiety is a phosphodiester linkage. In some embodiments, a linking moiety is an H-phosphonate moiety. In some embodiments, a linking moiety is a modified phosphorus linkage as described herein. In some embodiments, a universal linker (UnyLinker) is used to attached the oligonucleotide to the solid support (Ravikumar et al., Org. Process Res. Dev., 2008, 12 (3), 399-410). In some embodiments, other universal linkers are used (Pon, R. T., Curr. Prot. Nucleic Acid Chem., 2000, 3.1.1-3.1.28). In some embodiments, various orthogonal linkers (such as disulfide linkers) are used (Pon, R. T., Curr. Prot. Nucleic Acid Chem., 2000, 3.1.1-3.1.28).

Among other things, the present disclosure recognizes that a linker can be chosen or designed to be compatible with a set of reaction conditions employed in single-stranded RNAi agent synthesis. In some embodiments, to avoid degradation of single-stranded RNAi agents and to avoid desulfurization, auxiliary groups are selectively removed before de-protection. In some embodiments, DPSE group can selectively be removed by F⁻ ions. In some embodiments, the present disclosure provides linkers that are stable under a DPSE de-protection condition, e.g., 0.1M TBAF in MeCN, 0.5M HF-Et₃N in THF or MeCN, etc. In some embodiments, a provided linker is the SP linker. In some embodiments, the present disclosure demonstrates that the SP linker is stable under a DPSE de-protection condition, e.g., 0.1M TBAF in MeCN, 0.5M HF-Et₃N in THF or MeCN, etc.; they are also stable, e.g., under anhydrous basic conditions, such as om1M DBU in MeCN.

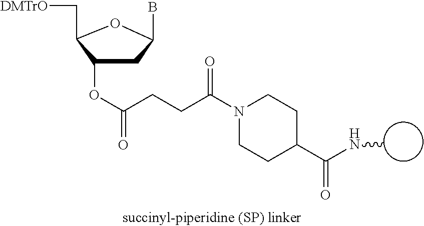

In some embodiments, an example linker is:

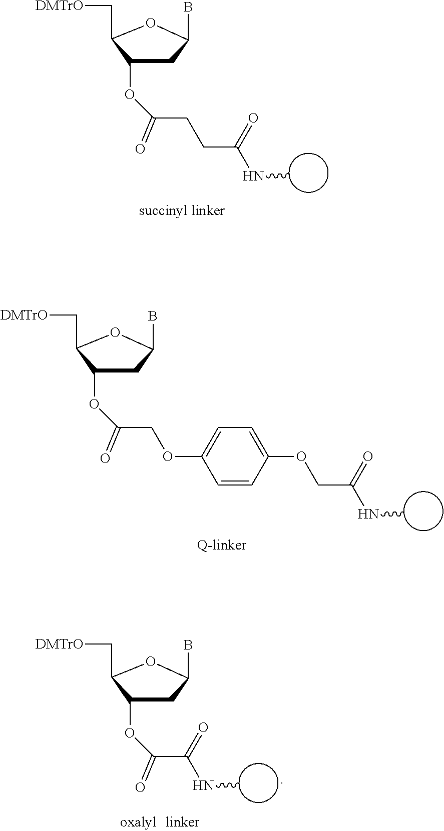

In some embodiments, the succinyl linker, Q-linker or oxalyl linker is not stable to one or more DPSE-deprotection conditions using F⁻.

### General Conditions—Solvents for Synthesis

Syntheses of provided oligonucleotides are generally performed in aprotic organic solvents. In some embodiments, a solvent is a nitrile solvent such as, e.g., acetonitrile. In some embodiments, a solvent is a basic amine solvent such as, e.g., pyridine. In some embodiments, a solvent is an ethereal solvent such as, e.g., tetrahydrofuran. In some embodiments, a solvent is a halogenated hydrocarbon such as, e.g., dichloromethane. In some embodiments, a mixture of solvents is used. In certain embodiments a solvent is a mixture of any one or more of the above-described classes of solvents.

In some embodiments, when an aprotic organic solvent is not basic, a base is present in the reacting step. In some embodiments where a base is present, the base is an amine base such as, e.g., pyridine, quinoline, or N,N-dimethylaniline. Example other amine bases include pyrrolidine, piperidine, N-methyl pyrrolidine, pyridine, quinoline, N,N-dimethylaminopyridine (DMAP), or N,N-dimethylaniline.

In some embodiments, a base is other than an amine base.

In some embodiments, an aprotic organic solvent is anhydrous. In some embodiments, an anhydrous aprotic organic solvent is freshly distilled. In some embodiments, a freshly distilled anhydrous aprotic organic solvent is a basic amine solvent such as, e.g., pyridine. In some embodiments, a freshly distilled anhydrous aprotic organic solvent is an ethereal solvent such as, e.g., tetrahydrofuran. In some embodiments, a freshly distilled anhydrous aprotic organic solvent is a nitrile solvent such as, e.g., acetonitrile.

### Chiral Reagent/Chiral Auxiliary

In some embodiments, chiral reagents are used to confer stereoselectivity in the production of chirally controlled oligonucleotides. Many different chiral reagents, also referred to by those of skill in the art and herein as chiral auxiliaries, may be used in accordance with methods of the present disclosure. Examples of such chiral reagents are described herein and in Wada I, II and III, referenced above. In some embodiments of the disclosure, a chiral reagent is a compound of one of the following formulae:

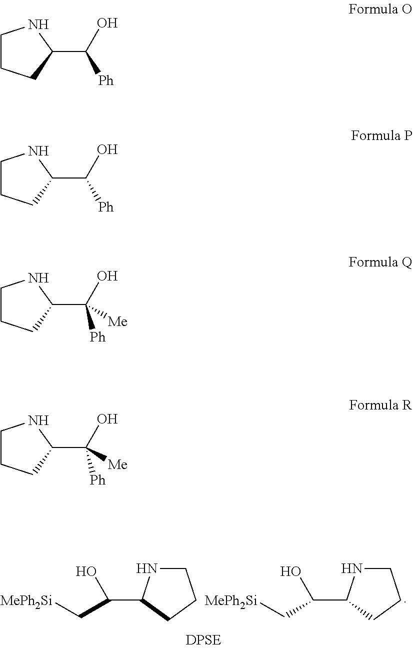

As demonstrated herein, when used for preparing a chiral internucleotidic linkage, to obtain stereoselectivity generally stereochemically pure chiral reagents are utilized.

Additional chiral auxiliaries and their use can be found in e.g., Wada I (JP4348077; WO2005/014609; WO2005/092909), Wada II (WO2010/064146), Wada III (WO2012/039448), Chiral Control (WO2010/064146), etc.

### Activation

An achiral H-phosphonate moiety is treated with the first activating reagent to form the first intermediate. In one embodiment, the first activating reagent is added to the reaction mixture during the condensation step. Use of the first activating reagent is dependent on reaction conditions such as solvents that are used for the reaction. Examples of the first activating reagent are phosgene, trichloromethyl chloroformate, bis(trichloromethyl)carbonate (BTC), oxalyl chloride, Ph₃PCl₂, (PhO)₃PCl₂, N,N′-bis(2-oxo-3-oxazolidinyl)phosphinic chloride (BopCl), 1,3-dimethyl-2-(3-nitro-1,2,4-triazol-1-yl)-2-pyrrolidin-1-yl-1,3,2-diazaphospholidinium hexafluorophosphate (MNTP), or 3-nitro-1,2,4-triazol-1-yl-tris(pyrrolidin-1-yl)phosphonium hexafluorophosphate (PyNTP).

The example of achiral H-phosphonate moiety is a compound shown in the above Scheme. DBU represents 1,8-diazabicyclo[5.4.0]undec-7-ene. H⁺DBU may be, for example, ammonium ion, alkylammonium ion, heteroaromatic iminium ion, or heterocyclic iminium ion, any of which is primary, secondary, tertiary or quaternary, or a monovalent metal ion.

Reacting with Chiral Reagent

After the first activation step, the activated achiral H-phosphonate moiety reacts with a chiral reagent.

### Stereospecific Condensation Step

A chiral intermediate is treated with the second activating reagent and a nucleoside to form a condensed intermediate. The nucleoside may be on solid support. Examples of the second activating reagent are 4,5-dicyanoimidazole (DCI), 4,5-dichloroimidazole, 1-phenylimidazolium triflate (PhIMT), benzimidazolium triflate (BIT), benztriazole, 3-nitro-1,2,4-triazole (NT), tetrazole, 5-ethylthiotetrazole (ETT), 5-benzylthiotetrazole (BTT), 5-(4-nitrophenyl)tetrazole, N-cyanomethylpyrrolidinium triflate (CMPT), N-cyanomethylpiperidinium triflate, N-cyanomethyldimethylammonium triflate. A chiral intermediate of Formula Z-Va ((Z-Vb), (Z-Va′), or (Z-Vb′)) may be isolated as a monomer. Usually, the chiral intermediate of Z-Va ((Z-Vb), (Z-Va′), or (Z-Vb′)) is not isolated and undergoes a reaction in the same pot with a nucleoside or modified nucleoside to provide a chiral phosphite compound, a condensed intermediate. In other embodiments, when the method is performed via solid phase synthesis, the solid support comprising the compound is filtered away from side products, impurities, and/or reagents.

### Capping Step

If the final nucleic acid is larger than a dimer, the unreacted —OH moiety is capped with a blocking group and the chiral auxiliary in the compound may also be capped with a blocking group to form a capped condensed intermediate. If the final nucleic acid is a dimer, then the capping step is not necessary.

### Modifying Step

The compound is modified by reaction with an electrophile. The capped condensed intermediate may be executed modifying step. In some embodiments, the modifying step is performed using a sulfur electrophile, a selenium electrophile or a boronating agent. Examples of modifying steps are step of oxidation and sulfurization.

In some embodiments of the method, the sulfur electrophile is a compound having one of the following formulas:

Z^{z1}—S—S—Z^{z2}, or Z^{z1}—S-_{Vz}-Z^{z2}; S₈ (Formula Z-B),

wherein Z^{z1} and Z^{z2} are independently alkyl, aminoalkyl, cycloalkyl, heterocyclic, cycloalkylalkyl, heterocycloalkyl, aryl, heteroaryl, alkyloxy, aryloxy, heteroaryloxy, acyl, amide, imide, or thiocarbonyl, or Z^{z1} and Z^{z2} are taken together to form a 3 to 8 membered alicyclic or heterocyclic ring, which may be substituted or unsubstituted; v_{z} is SO₂, O, or NR^{f}; and R^{f} is hydrogen, alkyl, alkenyl, alkynyl, or aryl.

Additional sulfur electrophiles are known in the art.

In some embodiments, after the modifying step, a chiral auxiliary group falls off from the growing oligonucleotide chain. In some embodiments, after the modifying step, a chiral auxiliary group remains connected to the internucleotidic phosphorus atom.

In some embodiments of the method, the modifying step is an oxidation step. In some embodiments of the method, the modifying step is an oxidation step using similar conditions as described above in this application. In some embodiments, an oxidation step is as disclosed in, e.g., JP 2010-265304 A and WO2010/064146.

### Chain Elongation Cycle and De-Protection Step

The capped condensed intermediate is deblocked to remove the blocking group at the 5′-end of the growing nucleic acid chain to provide a compound. The compound is optionally allowed to re-enter the chain elongation cycle to form a condensed intermediate, a capped condensed intermediate, a modified capped condensed intermediate, and a 5′-deprotected modified capped intermediate. Following at least one round of chain elongation cycle, the 5′-deprotected modified capped intermediate is further deblocked by removal of the chiral auxiliary ligand and other protecting groups for, e.g., nucleobase, modified nucleobase, sugar and modified sugar protecting groups, to provide a nucleic acid. In other embodiments, the nucleoside comprising a 5′-OH moiety is an intermediate from a previous chain elongation cycle as described herein. In yet other embodiments, the nucleoside comprising a 5′-OH moiety is an intermediate obtained from another known nucleic acid synthetic method. In embodiments where a solid support is used, the phosphorus-atom modified nucleic acid is then cleaved from the solid support. In certain embodiments, the nucleic acids is left attached on the solid support for purification purposes and then cleaved from the solid support following purification.

In yet other embodiments, the nucleoside comprising a 5′-OH moiety is an intermediate obtained from another known nucleic acid synthetic method. In yet other embodiments, the nucleoside comprising a 5′-OH moiety is an intermediate obtained from another known nucleic acid synthetic method as described in this application. In yet other embodiments, the nucleoside comprising a 5′-OH moiety is an intermediate obtained from another known nucleic acid synthetic method comprising one or more cycles illustrated in Scheme I. In some embodiments, the present disclosure provides oligonucleotide synthesis methods that use stable and commercially available materials as starting materials. In some embodiments, the present disclosure provides oligonucleotide synthesis methods to produce stereocontrolled phosphorus atom-modified oligonucleotide derivatives using an achiral starting material.

In some embodiments, the method of the present disclosure does not cause degradations under the de-protection steps. Further the method does not require special capping agents to produce phosphorus atom-modified oligonucleotide derivatives.

### Condensing Reagent

Condensing reagents (C_{R}) useful in accordance with methods of the present disclosure are of any one of the following general formulae:

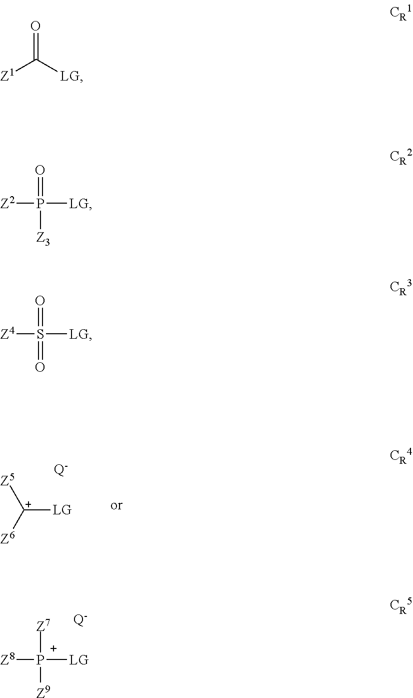

wherein Z¹, Z², Z³, Z⁴, Z⁵, Z⁶, Z⁷, Z⁸, and Z⁹ are independently optionally substituted group selected from alkyl, aminoalkyl, cycloalkyl, heterocyclic, cycloalkylalkyl, heterocycloalkyl, aryl, heteroaryl, alkyloxy, aryloxy, or heteroaryloxy, or wherein any of Z² and Z³, Z⁵ and Z⁶, Z⁷ and Z⁸, Z⁸ and Z⁹, Z⁹ and Z⁷, or Z⁷ and Z⁸ and Z⁹ are taken together to form a 3 to 20 membered alicyclic or heterocyclic ring; Q⁻ is a counter anion; and LG is a leaving group.

In some embodiments, a counter ion of a condensing reagent C_{R} is Cl⁻, Br⁻, BF₄⁻, PF₆⁻, TfO⁻, Tf₂N⁻, AsF₆⁻, ClO₄⁻, or SbF₆⁻, wherein Tf is CF₃SO₂. In some embodiments, a leaving group of a condensing reagent C_{R} is F, Cl, Br, I, 3-nitro-1,2,4-triazole, imidazole, alkyltriazole, tetrazole, pentafluorobenzene, or 1-hydroxybenzotriazole.

In some embodiments, a condensing reagent is selected from those described in WO/2006/066260.

In some embodiments, a condensing reagent is 1,3-dimethyl-2-(3-nitro-1,2,4-triazol-1-yl)-2-pyrrolidin-1-yl-1,3,2-diazaphospholidinium hexafluorophosphate (MNTP), or 3-nitro-1,2,4-triazol-1-yl-tris(pyrrolidin-1-yl)phosphonium hexafluorophosphate (PyNTP):

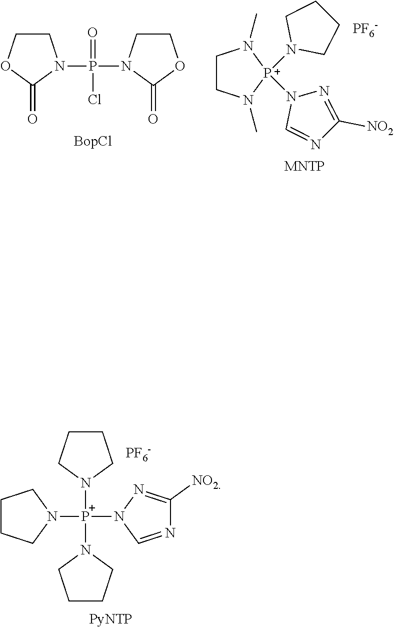

### Selection of Base and Sugar of Nucleoside Coupling Partner

As described herein, nucleoside coupling partners for use in accordance with methods of the present disclosure can be the same as one another or can be different from one another. In some embodiments, nucleoside coupling partners for use in the synthesis of a provided oligonucleotide are of the same structure and/or stereochemical configuration as one another. In some embodiments, each nucleoside coupling partner for use in the synthesis of a provided oligonucleotide is not of the same structure and/or stereochemical configuration as certain other nucleoside coupling partners of the oligonucleotide. Example nucleobases and sugars for use in accordance with methods of the present disclosure are described herein. One of skill in the relevant chemical and synthetic arts will recognize that any combination of nucleobases and sugars described herein are contemplated for use in accordance with methods of the present disclosure.

### Coupling Step

Example coupling procedures and chiral reagents and condensing reagents for use in accordance with the present disclosure are outlined in, inter alia, Wada I (JP4348077; WO2005/014609; WO2005/092909), Wada II (WO2010/064146), Wada III (WO2012/039448), and Chiral Control (WO2010/064146). Chiral nucleoside coupling partners for use in accordance with the present disclosure are also referred to herein as “Wada amidites.” In some embodiments, a coupling partner has the structure of

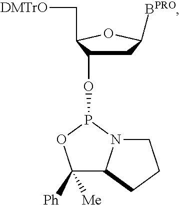

wherein B^{PRO} is a protected nucleobase. In some embodiments, a coupling partner has the structure of

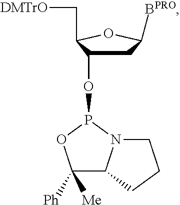

wherein B^{PRO} is a protected nucleobase. In some embodiments, a coupling partner has the structure of

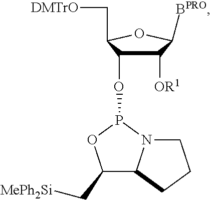

wherein B^{PRO} is a protected nucleobase, and R¹ is as defined and described herein. In some embodiments, a coupling partner has the structure of

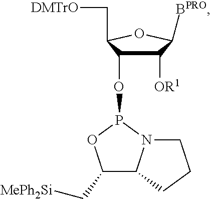

wherein B^{PRO} is a protected nucleobase, and R¹ is as defined and described herein. In some embodiments, R¹ is optionally substituted C₁₋₆ alkyl. In some embodiments, R¹ is Me.

Additional examples are described in WO/2010/064146, WO/2011/005761, WO/2013/012758, WO/2014/010250, US2013/0178612, WO/2014/012081, WO/2015/107425, WO/2017/015555, and WO/2017/062862, the phosphoramidites of each of which is incorporated herein by reference.

In some embodiments, the step of coupling comprises reacting a free hydroxyl group of a nucleotide unit of an APOC3 oligonucleotide with a nucleoside coupling partner under suitable conditions to effect the coupling. In some embodiments, the step of coupling is preceded by a step of deblocking. For instance, in some embodiments, the 5′ hydroxyl group of the growing oligonucleotide is blocked (i.e., protected) and must be deblocked in order to subsequently react with a nucleoside coupling partner.

Once the appropriate hydroxyl group of the growing oligonucleotide has been deblocked, the support is washed and dried in preparation for delivery of a solution comprising a chiral reagent and a solution comprising an activator. In some embodiments, a chiral reagent and an activator are delivered simultaneously. In some embodiments, co-delivery comprises delivering an amount of a chiral reagent in solution (e.g., a phosphoramidite solution) and an amount of activator in a solution (e.g., a CMPT solution) in a polar aprotic solvent such as a nitrile solvent (e.g., acetonitrile).

In some embodiments, the step of coupling provides a crude product composition in which the chiral phosphite product is present in a diastereomeric excess of >95%. In some embodiments, the chiral phosphite product is present in a diastereomeric excess of >96%. In some embodiments, the chiral phosphite product is present in a diastereomeric excess of >97%. In some embodiments, the chiral phosphite product is present in a diastereomeric excess of >98%. In some embodiments, the chiral phosphite product is present in a diastereomeric excess of >99%.

### Capping Step:

Provided methods for making chirally controlled oligonucleotides comprise a step of capping. In some embodiments, a step of capping is a single step. In some embodiments, a step of capping is two steps. In some embodiments, a step of capping is more than two steps. In some embodiments, a step of capping comprises steps of capping the free amine of the chiral auxiliary and capping any residual unreacted 5′ hydroxyl groups. In some embodiments, the free amine of the chiral auxiliary and the unreacted 5′ hydroxyl groups are capped with the same capping group. In some embodiments, the free amine of the chiral auxiliary and the unreacted 5′ hydroxyl groups are capped with different capping groups. In certain embodiments, capping with different capping groups allows for selective removal of one capping group over the other during synthesis of the oligonucleotide. In some embodiments, the capping of both groups occurs simultaneously. In some embodiments, the capping of both groups occurs iteratively. In certain embodiments, capping occurs iteratively and comprises a first step of capping the free amine followed by a second step of capping the free 5′ hydroxyl group, wherein both the free amine and the 5′ hydroxyl group are capped with the same capping group. For instance, in some embodiments, the free amine of the chiral auxiliary is capped using an anhydride (e.g., phenoxyacetic anhydride, i.e., Pac₂O) prior to capping of the 5′ hydroxyl group with the same anhydride. In certain embodiments, the capping of the 5′ hydroxyl group with the same anhydride occurs under different conditions (e.g., in the presence of one or more additional reagents). In some embodiments, capping of the 5′ hydroxyl group occurs in the presence of an amine base in an etherial solvent (e.g., NMI (N-methylimidazole) in THF). The phrase “capping group” is used interchangeably herein with the phrases “protecting group” and “blocking group”. In some embodiments, an amine capping group is characterized in that it effectively caps the amine such that it prevents rearrangement and/or decomposition of the intermediate phosphite species. In some embodiments, a capping group is selected for its ability to protect the amine of the chiral auxiliary in order to prevent intramolecular cleavage of the internucleotide linkage phosphorus. In some embodiments, a 5′ hydroxyl group capping group is characterized in that it effectively caps the hydroxyl group such that it prevents the occurrence of “shortmers,” e.g., “n−m” (m and n are integers and m<n; n is the number of bases in the targeted oligonucleotide) impurities that occur from the reaction of an APOC3 oligonucleotide chain that fails to react in a first cycle but then reacts in one or more subsequent cycles. The presence of such shortmers, especially “n−1”, has a deleterious effect upon the purity of the crude oligonucleotide and makes final purification of the oligonucleotide tedious and generally low-yielding. In some embodiments, a particular cap is selected based on its tendency to facilitate a particular type of reaction under particular conditions. For instance, in some embodiments, a capping group is selected for its ability to facilitate an E1 elimination reaction, which reaction cleaves the cap and/or auxiliary from the growing oligonucleotide. In some embodiments, a capping group is selected for its ability to facilitate an E2 elimination reaction, which reaction cleaves the cap and/or auxiliary from the growing oligonucleotide. In some embodiments, a capping group is selected for its ability to facilitate a (3-elimination reaction, which reaction cleaves the cap and/or auxiliary from the growing oligonucleotide.

### Modifying Step:

As used herein, the phrase “modifying step”, “modification step” and “P-modification step” are used interchangeably and refer generally to any one or more steps used to install a modified internucleotidic linkage. In some embodiments, the modified internucleotidic linkage having the structure of Formula I. A P-modification step of the present disclosure occurs during assembly of a provided oligonucleotide rather than after assembly of a provided oligonucleotide is complete. Thus, each nucleotide unit of a provided oligonucleotide can be individually modified at the linkage phosphorus during the cycle within which the nucleotide unit is installed. In some embodiments, a suitable P-modification reagent is a sulfur electrophile, selenium electrophile, oxygen electrophile, boronating reagent, or an azide reagent.

For instance, in some embodiments, a selenium reagent is elemental selenium, a selenium salt, or a substituted diselenide. In some embodiments, an oxygen electrophile is elemental oxygen, peroxide, or a substituted peroxide. In some embodiments, a boronating reagent is a borane-amine (e.g., N,N-diisopropylethylamine (BH₃.DIPEA), borane-pyridine (BH₃.Py), borane-2-chloropyridine (BH₃.CPy), borane-aniline (BH₃.An)), a borane-ether reagent (e.g., borane-tetrahydrofuran (BH₃.THF)), a borane-dialkylsulfide reagent (e.g., BH₃. Me₂S), aniline-cyanoborane, or a triphenylphosphine-carboalkoxyborane. In some embodiments, an azide reagent is comprises an azide group capable of undergoing subsequent reduction to provide an amine group.

In some embodiments, a P-modification reagent is a sulfurization reagent as described herein. In some embodiments, a step of modifying comprises sulfurization of phosphorus to provide a phosphorothioate linkage or phosphorothioate triester linkage. In some embodiments, a step of modifying provides an APOC3 oligonucleotide having an internucleotidic linkage of Formula I.

In some embodiments, the present disclosure provides sulfurizing reagents, and methods of making, and use of the same.

In some embodiments, such sulfurizing reagents are thiosulfonate reagents.

Various sulfurizing reagents and thiosulfonate reagents are known in the art.

In some embodiments, a sulfurization reagent for use in accordance with the present disclosure is characterized in that the moiety transferred to phosphorus during sulfurization is a substituted sulfur (e.g., —SR) as opposed to a single sulfur atom (e.g., —S⁻ or ═S).

In some embodiments, a sulfurization reagent for use in accordance with the present disclosure is characterized in that the activity of the reagent is tunable by modifying the reagent with a certain electron withdrawing or donating group.

In some embodiments, a sulfurization reagent for use in accordance with the present disclosure is characterized in that it is crystalline. In some embodiments, a sulfurization reagent for use in accordance with the present disclosure is characterized in that it has a high degree of crystallinity. In certain embodiments, a sulfurization reagent for use in accordance with the present disclosure is characterized by ease of purification of the reagent via, e.g., recrystallization. In certain embodiments, a sulfurization reagent for use in accordance with the present disclosure is characterized in that it is substantially free from sulfur-containing impurities. In some embodiments, sulfurization reagents which are substantially free from sulfur-containing impurities show increased efficiency.

In some embodiments, the provided chirally controlled oligonucleotide comprises one or more phosphate diester linkages. To synthesize such chirally controlled oligonucleotides, one or more modifying steps are optionally replaced with an oxidation step to install the corresponding phosphate diester linkages. In some embodiments, the oxidation step is performed in a fashion similar to ordinary oligonucleotide synthesis. In some embodiments, an oxidation step comprises the use of 12. In some embodiments, an oxidation step comprises the use of I₂ and pyridine. In some embodiments, an oxidation step comprises the use of 0.02 M I₂ in a THF/pyridine/water (70:20:10—v/v/v) co-solvent system. An example cycle is depicted in Scheme I-c.

In some embodiments, a phosphorothioate is directly formed through sulfurization by a sulfurization reagents, e.g., 3-phenyl-1,2,4-dithiazolin-5-one. In some embodiments, after a direct installation of a phosphorothioate, a chiral auxiliary group remains attached to the internucleotidic phosphorus atom. In some embodiments, an additional de-protecting step is required to remove the chiral auxiliary (e.g., for DPSE-type chiral auxiliary, using TBAF, HF-Et₃N, etc.).

In some embodiments, a phosphorothioate precursor is used to synthesize chirally controlled oligonucleotides comprising phosphorothioate linkages.

In some embodiments, the provided chirally controlled oligonucleotide comprises one or more phosphate diester linkages and one or more phosphorothioate diester linkages. In some embodiments, the provided chirally controlled oligonucleotide comprises one or more phosphate diester linkages and one or more phosphorothioate diester linkages, wherein at least one phosphate diester linkage is installed after all the phosphorothioate diester linkages when synthesized from 3′ to 5′. To synthesize such chirally controlled oligonucleotides, in some embodiments, one or more modifying steps are optionally replaced with an oxidation step to install the corresponding phosphate diester linkages, and a phosphorothioate precursor is installed for each of the phosphorothioate diester linkages. In some embodiments, a phosphorothioate precursor is converted to a phosphorothioate diester linkage after the desired oligonucleotide length is achieved. In some embodiments, the deprotection/release step during or after cycle exit converts the phosphorothioate precursors into phosphorothioate diester linkages.

In some embodiments, a phosphorothioate precursor is a phosphorus protecting group as described herein, e.g., 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, 4-[N-methyl-N-(2,2,2-trifluoroacetyl)amino]butyl. Examples are further depicted below.

As noted above, in some embodiments, sulfurization occurs under conditions which cleave the chiral reagent from the growing oligonucleotide. In some embodiments, sulfurization occurs under conditions which do not cleave the chiral reagent from the growing oligonucleotide.

In some embodiments, a sulfurization reagent is dissolved in a suitable solvent and delivered to the column. In certain embodiments, the solvent is a polar aprotic solvent such as a nitrile solvent. In some embodiments, the solvent is acetonitrile. In some embodiments, a solution of sulfurization reagent is prepared by mixing a sulfurization reagent (e.g., a thiosulfonate derivative as described herein) with BSTFA (N,O-bis-trimethylsilyl-trifluoroacetamide) in a nitrile solvent (e.g., acetonitrile). In some embodiments, BSTFA is not included. For example, the present inventors have found that relatively more reactive sulfurization reagents of general formula R^{s2}—S—S(O)₂—R^{s3} can often successfully participate in sulfurization reactions in the absence of BSTFA. To give but one example, the inventors have demonstrated that where R^{s2} is p-nitrophenyl and R^{s3} is methyl then no BSTFA is required. In light of this disclosure, those skilled in the art will readily be able to determine other situations and/or sulfurization reagents that do not require BSTFA.

In some embodiments, the sulfurization step is performed at room temperature. In some embodiments, the sulfurization step is performed at lower temperatures such as about 0° C., about 5° C., about 10° C., or about 15° C. In some embodiments, the sulfurization step is performed at elevated temperatures of greater than about 20° C.

In some embodiments, a sulfurization reaction is run for about 1 minute to about 120 minutes. In some embodiments, a sulfurization reaction is run for about 1 minute to about 90 minutes. In some embodiments, a sulfurization reaction is run for about 1 minute to about 60 minutes. In some embodiments, a sulfurization reaction is run for about 1 minute to about 30 minutes. In some embodiments, a sulfurization reaction is run for about 1 minute to about 25 minutes. In some embodiments, a sulfurization reaction is run for about 1 minute to about 20 minutes. In some embodiments, a sulfurization reaction is run for about 1 minute to about 15 minutes. In some embodiments, a sulfurization reaction is run for about 1 minute to about 10 minutes. In some embodiments, a sulfurization reaction is run for about 5 minute to about 60 minutes.

In some embodiments, a sulfurization reaction is run for about 5 minutes. In some embodiments, a sulfurization reaction is run for about 10 minutes. In some embodiments, a sulfurization reaction is run for about 15 minutes. In some embodiments, a sulfurization reaction is run for about 20 minutes. In some embodiments, a sulfurization reaction is run for about 25 minutes. In some embodiments, a sulfurization reaction is run for about 30 minutes. In some embodiments, a sulfurization reaction is run for about 35 minutes. In some embodiments, a sulfurization reaction is run for about 40 minutes. In some embodiments, a sulfurization reaction is run for about 45 minutes. In some embodiments, a sulfurization reaction is run for about 50 minutes. In some embodiments, a sulfurization reaction is run for about 55 minutes. In some embodiments, a sulfurization reaction is run for about 60 minutes.

It was unexpectedly found that certain of the sulfurization modification products made in accordance with methods of the present disclosure are unexpectedly stable. In some embodiments, it the unexpectedly stable products are phosphorothioate triesters. In some embodiments, the unexpectedly stable products are chirally controlled oligonucleotides comprising one or more internucleotidic linkages having the structure of Formula I-c.

One of skill in the relevant arts will recognize that sulfurization methods described herein and sulfurization reagents described herein are also useful in the context of modifying H-phosphonate oligonucleotides such as those described in Wada II (WO2010/064146).

In some embodiments, the sulfurization reaction has a stepwise sulfurization efficiency that is at least about 80%, 85%, 90%, 95%, 96%, 97%, or 98%. In some embodiments, the sulfurization reaction provides a crude dinucleotide product composition that is at least 98% pure. In some embodiments, the sulfurization reaction provides a crude tetranucleotide product composition that is at least 90% pure. In some embodiments, the sulfurization reaction provides a crude dodecanucleotide product composition that is at least 70% pure. In some embodiments, the sulfurization reaction provides a crude icosanucleotide product composition that is at least 50% pure.

Once the step of modifying the linkage phosphorus is complete, the oligonucleotide undergoes another deblock step in preparation for re-entering the cycle. In some embodiments, a chiral auxiliary remains intact after sulfurization and is deblocked during the subsequent deblock step, which necessarily occurs prior to re-entering the cycle. The process of deblocking, coupling, capping, and modifying, are repeated until the growing oligonucleotide reaches a desired length, at which point the oligonucleotide can either be immediately cleaved from the solid support or left attached to the support for purification purposes and later cleaved. In some embodiments, one or more protecting groups are present on one or more of the nucleotide bases, and cleavage of the oligonucleotide from the support and deprotection of the bases occurs in a single step. In some embodiments, one or more protecting groups are present on one or more of the nucleotide bases, and cleavage of the oligonucleotide from the support and deprotection of the bases occurs in more than one step. In some embodiments, deprotection and cleavage from the support occurs under basic conditions using, e.g., one or more amine bases. In certain embodiments, the one or more amine bases comprise propyl amine. In certain embodiments, the one or more amine bases comprise pyridine.

In some embodiments, cleavage from the support and/or deprotection occurs at elevated temperatures of about 30° C. to about 90° C. In some embodiments, cleavage from the support and/or deprotection occurs at elevated temperatures of about 40° C. to about 80° C. In some embodiments, cleavage from the support and/or deprotection occurs at elevated temperatures of about 50° C. to about 70° C. In some embodiments, cleavage from the support and/or deprotection occurs at elevated temperatures of about 60° C. In some embodiments, cleavage from the support and/or deprotection occurs at ambient temperatures.

Example purification procedures are described herein and/or are known generally in the relevant arts.

Noteworthy is that the removal of the chiral auxiliary from the growing oligonucleotide during each cycle is beneficial for at least the reasons that (1) the auxiliary will not have to be removed in a separate step at the end of the oligonucleotide synthesis when potentially sensitive functional groups are installed on phosphorus; and (2) unstable phosphorus-auxiliary intermediates prone to undergoing side reactions and/or interfering with subsequent chemistry are avoided. Thus, removal of the chiral auxiliary during each cycle makes the overall synthesis more efficient.

While the step of deblocking in the context of the cycle is described above, additional general methods are included below.

### Deblocking Step

In some embodiments, the step of coupling is preceded by a step of deblocking. For instance, in some embodiments, the 5′ hydroxyl group of the growing oligonucleotide is blocked (i.e., protected) and must be deblocked in order to subsequently react with a nucleoside coupling partner.

In some embodiments, acidification is used to remove a blocking group. In some embodiments, the acid is a Brønsted acid or Lewis acid. Useful Brønsted acids are carboxylic acids, alkylsulfonic acids, arylsulfonic acids, phosphoric acid and its derivatives, phosphonic acid and its derivatives, alkylphosphonic acids and their derivatives, arylphosphonic acids and their derivatives, phosphinic acid, dialkylphosphinic acids, and diarylphosphinic acids which have a pKa (25° C. in water) value of −0.6 (trifluoroacetic acid) to 4.76 (acetic acid) in an organic solvent or water (in the case of 80% acetic acid). The concentration of the acid (1 to 80%) used in the acidification step depends on the acidity of the acid. Consideration to the acid strength must be taken into account as strong acid conditions will result in depurination/depyrimidination, wherein purinyl or pyrimidinyl bases are cleaved from ribose ring and or other sugar ring. In some embodiments, an acid is selected from R^{a1}COOH, R^{a1}SO₃H, R^{a3}SO₃H,

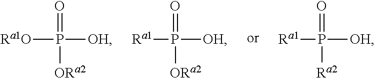

wherein each of R^{a1} and R^{a2} is independently hydrogen or an optionally substituted alkyl or aryl, and R^{a3} is an optionally substituted alkyl or aryl.

In some embodiments, acidification is accomplished by a Lewis acid in an organic solvent. Examples of such useful Lewis acids are Zn(X^{a})₂ wherein X^{a} is Cl, Br, I, or CF₃SO₃.

In some embodiments, the step of acidifying comprises adding an amount of a Brønsted or Lewis acid effective to remove a blocking group without removing purine moieties from the condensed intermediate.

Acids that are useful in the acidifying step also include, but are not limited to 10% phosphoric acid in an organic solvent, 10% hydrochloric acid in an organic solvent, 1% trifluoroacetic acid in an organic solvent, 3% dichloroacetic acid or trichloroacetic acid in an organic solvent or 80% acetic acid in water. The concentration of any Brønsted or Lewis acid used in this step is selected such that the concentration of the acid does not exceed a concentration that causes cleavage of a nucleobase from a sugar moiety.

In some embodiments, acidification comprises adding 1% trifluoroacetic acid in an organic solvent. In some embodiments, acidification comprises adding about 0.1% to about 8% trifluoroacetic acid in an organic solvent. In some embodiments, acidification comprises adding 3% dichloroacetic acid or trichloroacetic acid in an organic solvent. In some embodiments, acidification comprises adding about 0.1% to about 10% dichloroacetic acid or trichloroacetic acid in an organic solvent. In some embodiments, acidification comprises adding 3% trichloroacetic acid in an organic solvent. In some embodiments, acidification comprises adding about 0.1% to about 10% trichloroacetic acid in an organic solvent. In some embodiments, acidification comprises adding 80% acetic acid in water. In some embodiments, acidification comprises adding about 50% to about 90%, or about 50% to about 80%, about 50% to about 70%, about 50% to about 60%, about 70% to about 90% acetic acid in water. In some embodiments, the acidification comprises the further addition of cation scavengers to an acidic solvent. In certain embodiments, the cation scavengers can be triethylsilane or triisopropylsilane. In some embodiments, a blocking group is deblocked by acidification, which comprises adding 1% trifluoroacetic acid in an organic solvent. In some embodiments, a blocking group is deblocked by acidification, which comprises adding 3% dichloroacetic acid in an organic solvent. In some embodiments, a blocking group is deblocked by acidification, which comprises adding 3% trichloroacetic acid in an organic solvent. In some embodiments, a blocking group is deblocked by acidification, which comprises adding 3% trichloroacetic acid in dichloromethane.

In certain embodiments, methods of the present disclosure are completed on a synthesizer and the step of deblocking the hydroxyl group of the growing oligonucleotide comprises delivering an amount solvent to the synthesizer column, which column contains a solid support to which the oligonucleotide is attached. In some embodiments, the solvent is a halogenated solvent (e.g., dichloromethane). In certain embodiments, the solvent comprises an amount of an acid. In some embodiments, the solvent comprises an amount of an organic acid such as, for instance, trichloroacetic acid. In certain embodiments, the acid is present in an amount of about 1% to about 20% w/v. In certain embodiments, the acid is present in an amount of about 1% to about 10% w/v. In certain embodiments, the acid is present in an amount of about 1% to about 5% w/v. In certain embodiments, the acid is present in an amount of about 1 to about 3% w/v. In certain embodiments, the acid is present in an amount of about 3% w/v. Methods for deblocking a hydroxyl group are described further herein. In some embodiments, the acid is present in 3% w/v is dichloromethane.

In some embodiments, the chiral auxiliary is removed before the deblocking step. In some embodiments, the chiral auxiliary is removed during the deblocking step.

In some embodiments, cycle exit is performed before the deblocking step. In some embodiments, cycle exit is preformed after the deblocking step.

### General Conditions for Blocking Group/Protecting Group Removal

Functional groups such as hydroxyl or amino moieties which are located on nucleobases or sugar moieties are routinely blocked with blocking (protecting) groups (moieties) during synthesis and subsequently deblocked. In general, a blocking group renders a chemical functionality of a molecule inert to specific reaction conditions and can later be removed from such functionality in a molecule without substantially damaging the remainder of the molecule (see e.g., Green and Wuts, Protective Groups in Organic Synthesis, 2nd Ed., John Wiley & Sons, New York, 1991). For example, amino groups can be blocked with nitrogen blocking groups. Chemical functional groups can also be blocked by including them in a precursor form. Thus an azido group can be considered a blocked form of an amine as the azido group is easily converted to the amine. Further representative protecting groups utilized in nucleic acid synthesis are known (see e.g. Agrawal et al., Protocols for Oligonucleotide Conjugates, Eds., Humana Press, New Jersey, 1994, Vol. 26, pp. 1-72).

Various methods are known and used for removal of blocking groups from nucleic acids. In some embodiments, all blocking groups are removed. In some embodiments, a portion of blocking groups are removed. In some embodiments, reaction conditions can be adjusted to selectively remove certain blocking groups.

In some embodiments, nucleobase blocking groups, if present, are cleavable with an acidic reagent after the assembly of a provided oligonucleotide. In some embodiment, nucleobase blocking groups, if present, are cleavable under neither acidic nor basic conditions, e.g. cleavable with fluoride salts or hydrofluoric acid complexes. In some embodiments, nucleobase blocking groups, if present, are cleavable in the presence of base or a basic solvent after the assembly of a provided oligonucleotide. In certain embodiments, one or more of the nucleobase blocking groups are characterized in that they are cleavable in the presence of base or a basic solvent after the assembly of a provided oligonucleotide but are stable to the particular conditions of one or more earlier deprotection steps occurring during the assembly of the provided oligonucleotide.

In some embodiments, blocking groups for nucleobases are not required. In some embodiments, blocking groups for nucleobases are required. In some embodiments, certain nucleobases require one or more blocking groups while other nucleobases do not require one or more blocking groups.

In some embodiments, the oligonucleotide is cleaved from the solid support after synthesis. In some embodiments, cleavage from the solid support comprises the use of propylamine. In some embodiments, cleavage from the solid support comprises the use of propylamine in pyridine. In some embodiments, cleavage from the solid support comprises the use of 20% propylamine in pyridine. In some embodiments, cleavage from the solid support comprises the use of propylamine in anhydrous pyridine. In some embodiments, cleavage from the solid support comprises the use of 20% propylamine in anhydrous pyridine. In some embodiments, cleavage from the solid support comprises use of a polar aprotic solvent such as acetonitrile, NMP, DMSO, sulfone, and/or lutidine. In some embodiments, cleavage from the solid support comprises use of solvent, e.g., a polar aprotic solvent, and one or more primary amines (e.g., a C₁₋₁₀ amine), and/or one or more of methoxylamine, hydrazine, and pure anhydrous ammonia.

In some embodiments, deprotection of oligonucleotide comprises the use of propylamine. In some embodiments, deprotection of oligonucleotide comprises the use of propylamine in pyridine. In some embodiments, deprotection of oligonucleotide comprises the use of 20% propylamine in pyridine. In some embodiments deprotection of oligonucleotide comprises the use of propylamine in anhydrous pyridine. In some embodiments, deprotection of oligonucleotide comprises the use of 20% propylamine in anhydrous pyridine.

In some embodiments, the oligonucleotide is deprotected during cleavage.

In some embodiments, cleavage of oligonucleotide from solid support, or deprotection of oligonucleotide, is performed at about room temperature. In some embodiments, cleavage of oligonucleotide from solid support, or deprotection of oligonucleotide, is performed at elevated temperature. In some embodiments, cleavage of oligonucleotide from solid support, or deprotection of oligonucleotide, is performed at above about 30° C., 40° C., 50° C., 60° C., 70° C., 80° C. 90° C. or 100° C. In some embodiments, cleavage of oligonucleotide from solid support, or deprotection of oligonucleotide, is performed at about 30° C., 40° C., 50° C., 60° C., 70° C., 80° C. 90° C. or 100° C. In some embodiments, cleavage of oligonucleotide from solid support, or deprotection of oligonucleotide, is performed at about 40-80° C. In some embodiments, cleavage of oligonucleotide from solid support, or deprotection of oligonucleotide, is performed at about 50-70° C. In some embodiments, cleavage of oligonucleotide from solid support, or deprotection of oligonucleotide, is performed at about 60° C.

In some embodiments, cleavage of oligonucleotide from solid support, or deprotection of oligonucleotide, is performed for more than 0.1 hr, 1 hr, 2 hrs, 5 hrs, 10 hrs, 15 hrs, 20 hrs, 24 hrs, 30 hrs, or 40 hrs. In some embodiments, cleavage of oligonucleotide from solid support, or deprotection of oligonucleotide, is performed for about 0.1-5 hrs. In some embodiments, cleavage of oligonucleotide from solid support, or deprotection of oligonucleotide, is performed for about 3-10 hrs. In some embodiments, cleavage of oligonucleotide from solid support, or deprotection of oligonucleotide, is performed for about 5-15 hrs. In some embodiments, cleavage of oligonucleotide from solid support, or deprotection of oligonucleotide, is performed for about 10-20 hrs. In some embodiments, cleavage of oligonucleotide from solid support, or deprotection of oligonucleotide, is performed for about 15-25 hrs. In some embodiments, cleavage of oligonucleotide from solid support, or deprotection of oligonucleotide, is performed for about 20-40 hrs. In some embodiments, cleavage of oligonucleotide from solid support, or deprotection of oligonucleotide, is performed for about 2 hrs. In some embodiments, cleavage of oligonucleotide from solid support, or deprotection of oligonucleotide, is performed for about 5 hrs. In some embodiments, cleavage of oligonucleotide from solid support, or deprotection of oligonucleotide, is performed for about 10 hrs. In some embodiments, cleavage of oligonucleotide from solid support, or deprotection of oligonucleotide, is performed for about 15 hrs. In some embodiments, cleavage of oligonucleotide from solid support, or deprotection of oligonucleotide, is performed for about 18 hrs. In some embodiments, cleavage of oligonucleotide from solid support, or deprotection of oligonucleotide, is performed for about 24 hrs.

In some embodiments, cleavage of oligonucleotide from solid support, or deprotection of oligonucleotide, is performed at room temperature for more than 0.1 hr, 1 hr, 2 hrs, 5 hrs, 10 hrs, 15 hrs, 20 hrs, 24 hrs, 30 hrs, or 40 hrs. In some embodiments, cleavage of oligonucleotide from solid support, or deprotection of oligonucleotide, is performed at room temperature for about 5-48 hrs. In some embodiments, cleavage of oligonucleotide from solid support, or deprotection of oligonucleotide, is performed at room temperature for about 10-24 hrs. In some embodiments, cleavage of oligonucleotide from solid support, or deprotection of oligonucleotide, is performed at room temperature for about 18 hrs. In some embodiments, cleavage of oligonucleotide from solid support, or deprotection of oligonucleotide, is performed at elevated temperature for more than 0.1 hr, 1 hr, 2 hrs, 5 hrs, 10 hrs, 15 hrs, 20 hrs, 24 hrs, 30 hrs, or 40 hrs. In some embodiments, cleavage of oligonucleotide from solid support, or deprotection of oligonucleotide, is performed at elevated temperature for about 0.5-5 hrs. In some embodiments, cleavage of oligonucleotide from solid support, or deprotection of oligonucleotide, is performed at about 60° C. for about 0.5-5 hrs. In some embodiments, cleavage of oligonucleotide from solid support, or deprotection of oligonucleotide, is performed at about 60° C. for about 2 hrs.

In some embodiments, cleavage of oligonucleotide from solid support, or deprotection of oligonucleotide comprises the use of propylamine and is performed at room temperature or elevated temperature for more than 0.1 hr, 1 hr, 2 hrs, 5 hrs, 10 hrs, 15 hrs, 20 hrs, 24 hrs, 30 hrs, or 40 hrs. Example conditions are 20% propylamine in pyridine at room temperature for about 18 hrs, and 20% propylamine in pyridine at 60° C. for about 18 hrs.

In some embodiments, prior to cleavage from solid support, a step is performed to remove a chiral auxiliary group, if one is still attached to an internucleotidic phosphorus atom. In some embodiments, for example, one or more DPSE type chiral auxiliary groups remain attached to internucleotidic phosphorus atoms during the oligonucleotide synthesis cycle. Suitable conditions for removing remaining chiral auxiliary groups are widely known in the art, e.g., those described in Wada I, Wada II, Wada III, Chiral Control, etc. In some embodiments, a condition for removing DPSE type chiral auxiliary is TBAF or HF-Et₃N, e.g., 0.1M TBAF in MeCN, 0.5M HF-Et₃N in THF or MeCN, etc. In some embodiments, the present disclosure recognizes that a linker may be cleaved during the process of removing a chiral auxiliary group. In some embodiments, the present disclosure provides linkers, such as the SP linker, that provides better stability during chiral auxiliary group removal. Among other things, certain linkers provided by the present disclosure provided improved yield and/or purity.

In some embodiments, an example cycle is depicted in Scheme I.

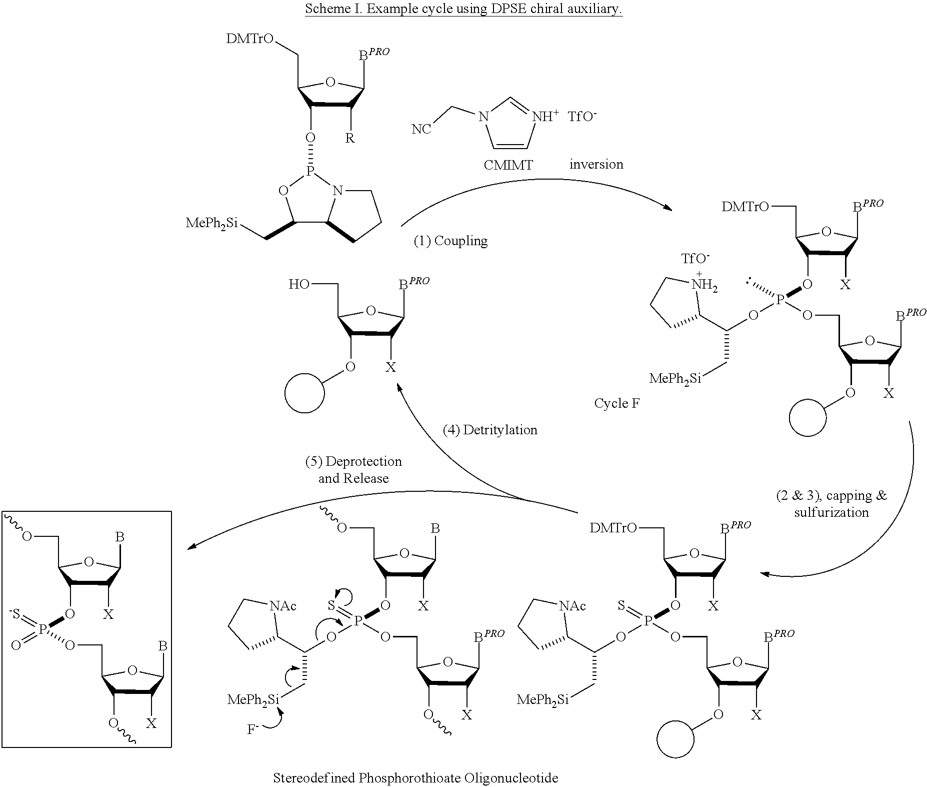

In some embodiments, X is H or a 2′-modification. In some embodiments, X is H or —OR¹, wherein R¹ is not hydrogen. In some embodiments, X is H or —OR¹, wherein R¹ is optionally substituted C₁₋₆ alkyl. In some embodiments, X is H. In some embodiments, X is —OMe. In some embodiments, X is —OCH₂CH₂OCH₃. In some embodiments, X is —F.

It is understood by a person having ordinary skill in the art that different types of cycles may be combined to provide complete control of the chemical modifications and stereochemistry of oligonucleotides. In some embodiments, for example, an APOC3 oligonucleotide synthesis process may contain one or more Cycles. In some embodiments, a provided method comprises at least one cycle using a DPSE-type chiral auxiliary.

In some embodiments, the present disclosure provides methods for preparing provided oligonucleotide and oligonucleotide compositions. In some embodiments, a provided method comprises the step of providing a provided chiral reagent having the structure of

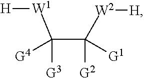

wherein W¹ is —NG⁵, W² is O, each of G¹ and G³ is independently hydrogen or an optionally substituted group selected from C₁₋₁₀ aliphatic, heterocyclyl, heteroaryl and aryl, G² is —C(R)₂Si(R)₃, and G⁴ and G⁵ are taken together to form an optionally substituted saturated, partially unsaturated or unsaturated heteroatom-containing ring of up to about 20 ring atoms which is monocyclic or polycyclic, fused or unfused, wherein each R is independently hydrogen, or an optionally substituted group selected from C₁-C₆ aliphatic, carbocyclyl, aryl, heteroaryl, and heterocyclyl. In some embodiments, a provided chiral reagent has the structure of

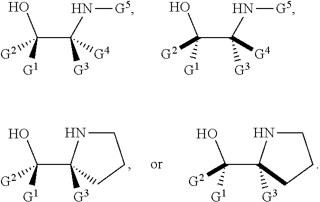

In some embodiments, a provided methods comprises providing a phosphoramidite comprising a moiety from a chiral reagent having the structure of

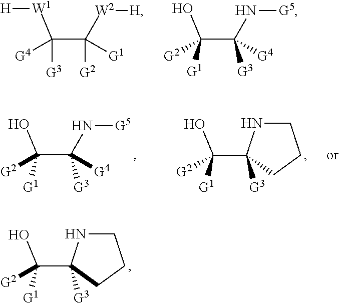

wherein —W¹H and —W²H, or the hydroxyl and amino groups, form bonds with the phosphorus atom of the phosphoramidite. In some embodiments, —W¹H and —W²H, or the hydroxyl and amino groups, form bonds with the phosphorus atom of the phosphoramidite, e.g., in

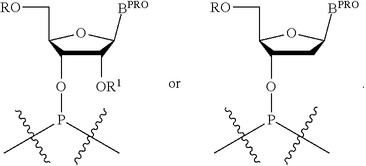

In some embodiments, a phosphoramidite has the structure of

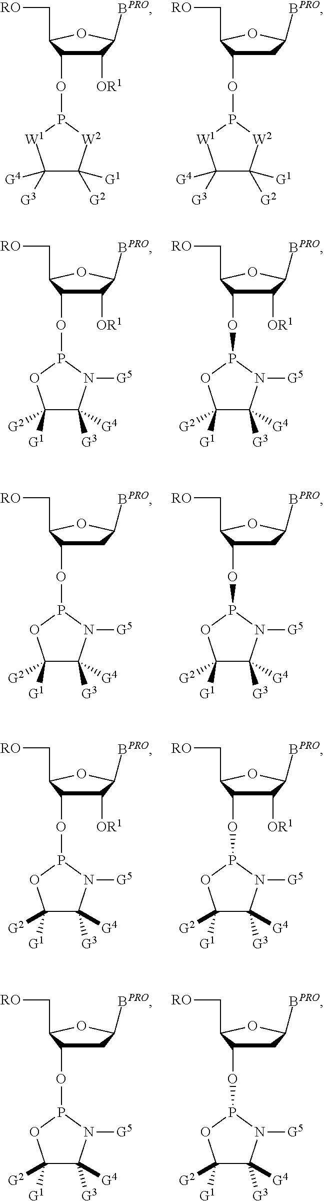

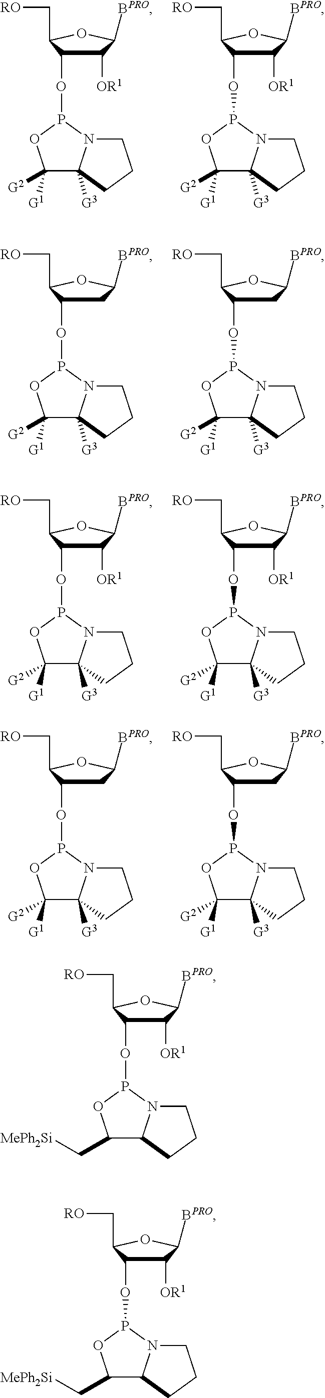

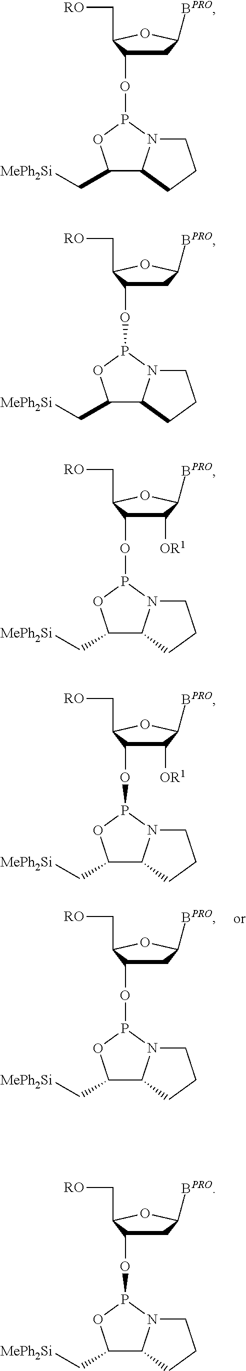

In some embodiments, R is a protection group. In some embodiments, R is DMTr. In some embodiments, G² is —C(R)₂Si(R)₃, wherein —C(R)₂— is optionally substituted —CH₂—, and each R of —Si(R)₃ is independently an optionally substituted group selected from C₁₋₁₀ aliphatic, heterocyclyl, heteroaryl and aryl. In some embodiments, at least one R of —Si(R)₃ is independently optionally substituted C₁₋₁₀ alkyl. In some embodiments, at least one R of —Si(R)₃ is independently optionally substituted phenyl. In some embodiments, one R of —Si(R)₃ is independently optionally substituted phenyl, and each of the other two R is independently optionally substituted C₁₋₁₀ alkyl. In some embodiments, one R of —Si(R)₃ is independently optionally substituted C₁₋₁₀ alkyl, and each of the other two R is independently optionally substituted phenyl. In some embodiments, G² is optionally substituted —CH₂Si(Ph)(Me)₂. In some embodiments, G² is optionally substituted —CH₂Si(Me)(Ph)₂. In some embodiments, G² is —CH₂Si(Me)(Ph)₂. In some embodiments, G⁴ and G⁵ are taken together to form an optionally substituted saturated 5-6 membered ring containing one nitrogen atom (to which G⁵ is attached). In some embodiments, G⁴ and G⁵ are taken together to form an optionally substituted saturated 5-membered ring containing one nitrogen atom. In some embodiments, G¹ is hydrogen. In some embodiments, G³ is hydrogen. In some embodiments, both G¹ and G³ are hydrogen. In some embodiments, both G¹ and G³ are hydrogen, G² is —C(R)₂Si(R)₃, wherein —C(R)₂— is optionally substituted —CH₂—, and each R of —Si(R)₃ is independently an optionally substituted group selected from C₁₋₁₀ aliphatic, heterocyclyl, heteroaryl and aryl, and G⁴ and G⁵ are taken together to form an optionally substituted saturated 5-membered ring containing one nitrogen atom. In some embodiments, a provided method further comprises providing a fluoro-containing reagent. In some embodiments, a provided fluoro-containing reagent removes a chiral reagent, or a product formed from a chiral reagent, from oligonucleotides after synthesis. Various known fluoro-containing reagents, including those F⁻sources for removing —SiR₃ groups, can be utilized in accordance with the present disclosure, for example, TBAF, HF₃-Et₃N etc. In some embodiments, a fluoro-containing reagent provides better results, for example, shorter treatment time, lower temperature, less de-sulfurization, etc, compared to traditional methods, such as concentrated ammonia. In some embodiments, for certain fluoro-containing reagent, the present disclosure provides linkers for improved results, for example, less cleavage of oligonucleotides from support during removal of chiral reagent (or product formed therefrom during oligonucleotide synthesis). In some embodiments, a provided linker is an SP linker. In some embodiments, the present disclosure demonstrated that a HF-base complex can be utilized, such as HF—NR³, to control cleavage during removal of chiral reagent (or product formed therefrom during oligonucleotide synthesis). In some embodiments, HF—NR³ is HF-NEt₃. In some embodiments, HF—NR₃ enables use of traditional linkers, e.g., succinyl linker.

In some embodiments, a method for production of an APOC3 oligonucleotide comprises at least one cycle using a DPSE-type chiral auxiliary, such as that shown in the following non-limiting example:

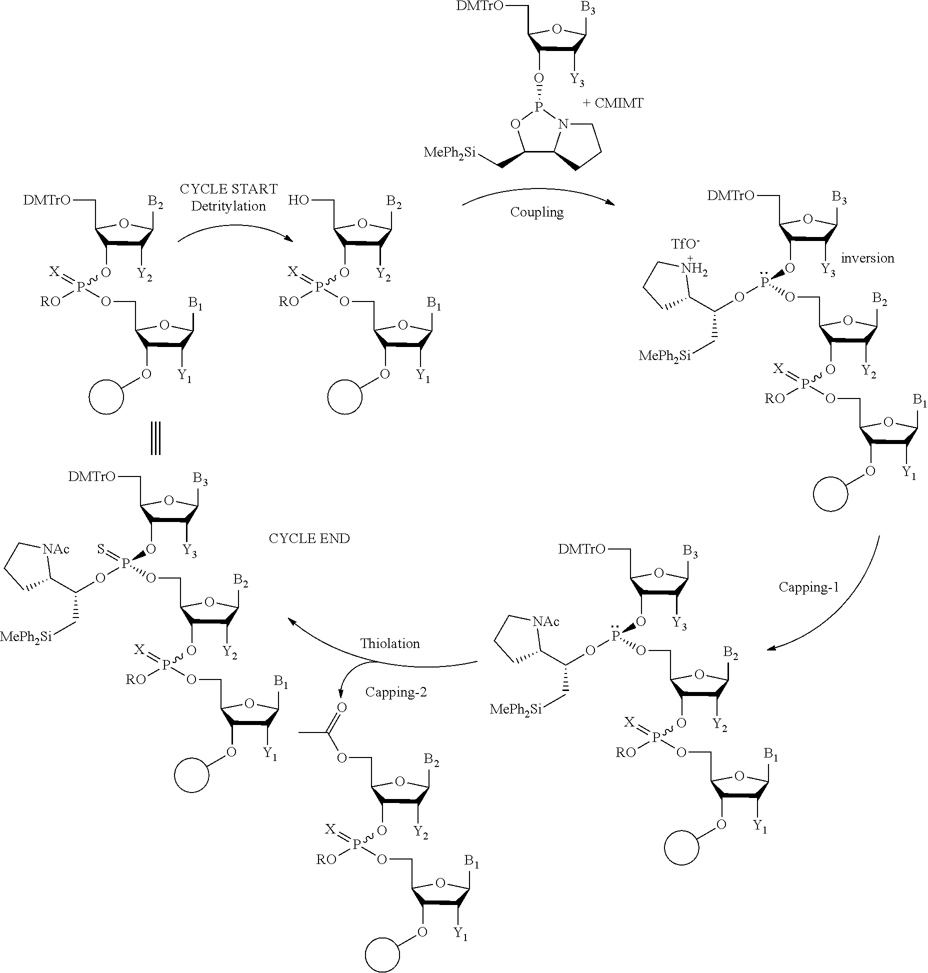

Detritylation:

The synthesis of an oligonucleotide started with 2′-F-U-DMTr loaded CPG solid support (3% dichloroacetic acid (DCA) in toluene was used for the removal of dimethoxy trityl group (DMTr) from the initial nucleobase attached on the solid support followed by an UV watch command mode at the wavelength of 436 nm. Linear flowrate, 424 cm/hr, used for detritylation.

Coupling:

For the coupling step, all amidites were dissolved either in acetonitrile (ACN) or in 20% isobutyronitrile (IBN)/ACN at a concentration of 0.2M; the solutions were dried over molecular sieves (3 Å) NLT 4h before use (10%, v/v). Dual activators (CMIMT and ETT) coupling approach were utilized for the manufacture of an oligonucleotide. Both activators were dissolved in ACN at a concentration of 0.6M. CMIMT has been used for the efficient coupling of stereo defined nucleotides and ETT is an activator used for the coupling of random/standard amidites/nucleotides. 2.5 equivalent of amidites used for coupling of stereo defined nucleotide over 10 min recycle time (linear recycle mode, 212 cm/hr). The molar ratio of CMIMT activator to stereo defined amidite was maintained at 6.1:1 (CMIMT/Amidite=6.1/1) in the coupling step. All random amidites were coupled for 8 min with ETT activator. The molar ratio of ETT to random/standard amidites was 4.5:1.

Cap 1:

Cap 1 is a step that is performed before thiolation. 1-1.5 CV Cap B solution is used over 4 min contact time for capping of the auxiliary amine on DPSE. Capping of DPSE chiral auxiliary with Cap B solution helps to reduce the content of early failure and post FLP impurities.

Thiolation:

Following the Cap 1 step, the phosphorous triester linkages, P(III), were stabilized with thiolating reagent, 0.2M xanthane hydride (XH) in pyridine, (0.6 CV) over 6 min contact time to form a stable P(V) bond.

Oxidation:

It is noted here that the Cap 1 step is not necessary for standard nucleotide cycle.

So, after coupling of standard nucleotides onto the solid support, the phosphorous triester linkages, P(III), were oxidized with 0.05M of iodine/water/pyridine solution (3.5 eq.) over 2 min contact time to form a stable P(V) bond.

Cap 2 (Post-Thio/Ox-Capping):

In general, 97-100% coupling efficiency was observed in the coupling step based on DMTr release cation. Residual uncoupled hydroxyl groups, typically 1-3% by detrit monitor, on the solid support were blocked with Cap A and Cap B solution using 0.4 CV over 0.8 min to prevent formation of deletion sequences. In case, any auxiliary amine remained un-capped in the pre-capping step will also be capped in this step.

Cycle Repeated

The synthetic cycle (DPSE cycle=Detritylation->Coupling->Cap 1->Thiolation->Cap2 and Standard cycle=Detritylation->Coupling->Oxidation->Cap2) was repeated until the desired length of oligonucleotide synthesized on the solid support.

In some embodiments, the present disclosure comprises a method for manufacturing an APOC3 oligonucleotide composition directed to a selected target sequence, the method comprising manufacturing a provided oligonucleotide composition capable of directing single-stranded RNA interference and comprising a first plurality of oligonucleotides, each of which has a base sequence complementary to the target sequence. In some embodiments, a provided method further comprises providing a pharmaceutically acceptable carrier.

As appreciated by a person having ordinary skill in the art, provided oligonucleotides can also be prepared through known solution phase synthesis using provided reagents and methods in accordance with the present disclosure.

As non-limiting examples, provided oligonucleotides can also be prepared through any process known in the art, including but not limited to, those described in: JP 4348044; WO2005092909; U.S. Pat. No. 9,394,333; WO2011005761; U.S. Pat. Nos. 8,470,987; 8,859,755; 8,822,671; WO2013012758; EP 13817386; WO2014012081; WO2015107425; WO2017015555; and WO2017062862.

Double-Stranded Oligonucleotides Comprising a Single-Stranded Oligonucleotide Disclosed Herein

In some embodiments, an APOC3 oligonucleotide is a single-stranded or double-stranded oligonucleotide. In some embodiments, the disclosure encompasses a double-stranded oligonucleotide or molecule comprising a single-stranded oligonucleotide as disclosed herein, and another oligonucleotide which is at least partially complementary to it. In some embodiments, the present disclosure pertains to compositions comprising such a double-stranded oligonucleotide.

In some embodiments, the disclosure encompasses a double-stranded molecule comprising a single-stranded oligonucleotide as disclosed herein, and another oligonucleotide which is at least partially complementary to it, e.g., one or both ends of the molecule has a 3′ or 5′ overhang.

In some embodiments, the disclosure encompasses a double-stranded molecule comprising a single-stranded oligonucleotide as disclosed herein, and another oligonucleotide which is fully complementary to it.

In some embodiments, the disclosure encompasses a double-stranded molecule capable of directing RNA interference comprising a single-stranded oligonucleotide as disclosed herein, and another oligonucleotide which is at least partially complementary to it.

In some embodiments, the disclosure encompasses a double-stranded molecule capable of directing RNA interference comprising a single-stranded oligonucleotide as disclosed herein, and another oligonucleotide which is at least partially complementary to it, e.g., one or both ends of the molecule capable of directing RNA interference has a 3′ or 5′ overhang.

In some embodiments, the disclosure encompasses a double-stranded molecule capable of directing RNA interference comprising a single-stranded oligonucleotide as disclosed herein, and another oligonucleotide which is fully complementary to it.

In some embodiments, the disclosure encompasses a double-stranded molecule capable of directing RNA interference and RNase H-mediated knockdown comprising a single-stranded oligonucleotide as disclosed herein, and another oligonucleotide which is at least partially complementary to it.

In some embodiments, the disclosure encompasses a double-stranded molecule capable of directing RNA interference and RNase H-mediated knockdown comprising a single-stranded oligonucleotide as disclosed herein, and another oligonucleotide which is at least partially complementary to it, e.g., one or both ends of the molecule capable of directing RNA interference and RNase H-mediated knockdown has a 3′ or 5′ overhang.

In some embodiments, the disclosure encompasses a double-stranded molecule capable of directing RNA interference and RNase H-mediated knockdown comprising a single-stranded oligonucleotide as disclosed herein, and another oligonucleotide which is fully complementary to it.

Provided first oligonucleotides and oligonucleotide compositions can have any format, structural element or base sequence of any oligonucleotide disclosed herein, and further comprise a second oligonucleotide or oligonucleotide strand at least partially complementary to the first oligonucleotide. In some embodiments, the first and/or second oligonucleotide can comprise any format, structural element or base sequence of (or a base sequence at least partially complementary to a base sequence of) any oligonucleotide disclosed herein. In some embodiments, a structural element is a 5′-end structure, 5′-end region, 5′-nucleotide, seed region, post-seed region, 3′-end region, 3′-terminal dinucleotide, 3′-end cap, or any portion of any of these structures, GC content, long GC stretch, and/or any modification, chemistry, stereochemistry, pattern of modification, chemistry or stereochemistry, or additional chemical moiety (e.g., including but not limited to, a targeting moiety, a lipid moiety, a GalNAc moiety, a carbohydrate moiety, etc.), any component, or any combination of any of the above.

### Biological Applications

As described herein, provided compositions and methods are capable of improving knockdown, including, single-stranded RNA interference of transcripts. In some embodiments, provided compositions and methods provide improved single-stranded RNA interference of transcripts compared to a reference pattern, which is a pattern from a reference condition selected from the group consisting of absence of the composition, presence of a reference composition, and combinations thereof. An improvement can be an improvement of any desired biological functions.

In some embodiments, the present disclosure provides a method for improving single-stranded RNA interference of a target transcript, comprising administering a composition comprising a first plurality of oligonucleotides, wherein the single-stranded RNA interference of the target transcript is improved relative to reference conditions selected from the group consisting of absence of the composition, presence of a reference composition, and combinations thereof.

In some embodiments, the present disclosure provides a method of mediating single-stranded RNA interference of a target, the method comprising steps of:

contacting a single-stranded RNA interference system containing the target transcript with an APOC3 oligonucleotide composition comprising a first plurality of oligonucleotides, in an amount, for a time, and under conditions sufficient for a set of single-stranded RNA interference products to be generated that is different from a set generated under reference conditions selected from the group consisting of absence of the composition, presence of a reference composition, and combinations thereof.

In some embodiments, the present disclosure provides compositions and methods for treating or preventing diseases, including but not limited to those described in references cited in this disclosure.

In some embodiments, the present disclosure provides a method for treating or preventing a disease, comprising administering to a subject an APOC3 oligonucleotide composition described herein.

In some embodiments, the present disclosure provides a method for treating or preventing a disease, comprising administering to a subject an APOC3 oligonucleotide composition comprising a first plurality of oligonucleotides, which:

1) have a common base sequence complementary to a target sequence in a transcript; and

2) comprise one or more modified sugar moieties and modified internucleotidic linkages,

the oligonucleotide composition being characterized in that, when it is contacted with the transcript in a single-stranded RNA interference system, RNAi-mediated knockdown of the transcript is improved relative to that observed under reference conditions selected from the group consisting of absence of the composition, presence of a reference composition, and combinations thereof.

In some embodiments, the present disclosure provides a method for treating or preventing a disease, comprising administering to a subject an APOC3 oligonucleotide composition comprising a first plurality of oligonucleotides capable of directing single-stranded RNA interference, wherein oligonucleotides are of a particular oligonucleotide type defined by:

1) base sequence;

2) pattern of backbone linkages;

3) pattern of backbone chiral centers; and

4) pattern of backbone phosphorus modifications,

which composition is chirally controlled in that it is enriched, relative to a substantially racemic preparation of oligonucleotides having the same base sequence, for oligonucleotides of the particular oligonucleotide type, wherein:

the oligonucleotide composition being characterized in that, when it is contacted with the transcript in a single-stranded RNA interference system, RNAi-mediated knockdown of the transcript is improved relative to that observed under reference conditions selected from the group consisting of absence of the composition, presence of a reference composition, and combinations thereof.

In some embodiments, a disease is one in which administering a provided composition capable of directing single-stranded RNA interference can repair, restore or introduce a new beneficial function.

In some embodiments, a disease is one in which, after administering a provided composition, a gene is effectively knocked down by improving single-stranded RNA interference system of the gene transcript.

In some embodiments, a disease is cancer.

In some embodiments, the present disclosure provides a method of treating a disease by administering a composition comprising a first plurality of oligonucleotides sharing a common base sequence comprising a common base sequence, which nucleotide sequence is complementary to a target sequence in the target transcript,

the improvement that comprises using as the oligonucleotide composition a stereocontrolled oligonucleotide composition characterized in that, when it is contacted with the transcript in an APOC3 oligonucleotide or a single-stranded RNA interference system, RNAi-mediated knockdown of the transcript is improved relative to that observed under reference conditions selected from the group consisting of absence of the composition, presence of a reference composition, and combinations thereof.

In some embodiments, a disease is cancer.

In some embodiments, a disease treatment comprises knockdown of a gene function by improving single-stranded RNA interference system.

In some embodiments, an APOC3 oligonucleotide or a single-stranded RNAi agent targets a target mRNA transcript.

In some embodiments, the common base sequence is capable of hybridizing with a transcript in a cell. In some embodiments, a common base sequence hybridizes with a transcript of any gene described herein or known in the art.

Treatment of Disorders

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion.

In some embodiments, provided oligonucleotides target APOC3.

In some embodiments, APOC3 is a gene, or a gene product thereof (including, but not limited to, a transcript or protein), also known as APOCIII, ApocIII, HALP2, apolipoprotein C3; OMIM: 107720; MGI: 88055; HomoloGene: 81615; or GeneCards: 345; or Human APOC3: Entrez 345; Ensembl: ENSG00000110245; UniProt: P02656; RefSeq (mRNA): NM_000040; RefSeq (protein): NP 000031.1; Location (UCSC): Chr 11:116.83-116.83 Mb; Mouse Entrez 11814; Ensembl: ENSMUSG00000032081; UniProt: P33622; RefSeq (mRNA): NM_023114 NM_001289755 NM_001289756 NM_001289833; RefSeq (protein): NP_001276685.1 NP_075603.1; Location (UCSC): Chr 9: 46.23-46.24 Mb. APOC3 reportedly inhibits lipoprotein lipase and hepatic lipase; it is reported to inhibit hepatic uptake of triglyceride-rich particles. An increase in APOC3 levels reportedly induces the development of hypertriglyceridemia. Scientific papers reportedly suggest an intracellular role for APOC3 in promoting the assembly and secretion of triglyceride-rich VLDL particles from hepatic cells under lipid-rich conditions. However, two naturally-occurring point mutations in human APOC3 coding sequence, namely Ala23Thr and Lys58Glu, reportedly abolish the intracellular assembly and secretion of triglyceride-rich VLDL particles from hepatic cells. Two novel susceptibility haplotypes (specifically, P2-52-X1 and P1-S2-X1) have been reportedly discovered in ApoAI-CIII-AIV gene cluster on chromosome 11q23; these reportedly confer approximately threefold higher risk of coronary heart disease in normal as well as non-insulin diabetes mellitus. APOC3 delays the catabolism of triglyceride rich particles. Elevations of APOC3 found in genetic variation studies may predispose patients to non-alcoholic fatty liver disease. APOC3 expression has reportedly been implicated in various disorders, including but not limited to: atherosclerosis or dyslipidemia, elevated triglyceride levels, elevated cholesterol levels, elevated free fatty acids, and diabetes. Vaith et al. 1978 Biochimica et Biophysica Acta. 541 (2): 234-40; Nicolardi et al. 2013 Journal of Proteome Research. 12 (5): 2260-8; Mendivil et al. 2010 Arteriosclerosis, Thrombosis, and Vascular Biology. 30 (2): 239-45; Sundaram et al. 2010 Journal of Lipid Research. 51 (1): 150-161; Sundaram et al. 2010 Journal of Lipid Research. 51 (6): 1524-1534; Qin et al. August 2011 The Journal of Biological Chemistry. 286 (31): 27769-27780; Singh et al. November 2008 International Journal of Cardiology. 130 (3): e93-5; Singh et al. June 2007 Diabetes & Vascular Disease Research. 4 (2): 124-29.

In some embodiments, an APOC3-related disorder is a disorder related to, caused and/or associated with abnormal or excessive activity, level and/or expression of, a deleterious mutation in, or abnormal tissue or inter- or intracellular distribution of an APOC3 gene or a gene product thereof. In some embodiments, non-limiting examples of an APOC3-related disorder include: heart disease, atherosclerosis, dyslipidemia, elevated triglyceride levels (hypertriglyceridemia), elevated cholesterol levels (hypercholesterolemia), cardiovascular disease, metabolic syndrome, obesity and diabetes, premature chronic heart disease (CHD), eruptive xanthoma, hepatosplenomegaly, pancreatitis, aneurysm, angina, arrhythmia, atherosclerosis, cerebrovascular disease (stroke), hypertension, and hyperlipidemia. In some embodiments, non-limiting examples of an APOC3-related disorder include: hyperlipidemia, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, angina pectoris, thrombosis, atherosclerosis, transient ischemic attacks, stroke, vascular restenosis, hyperglycemia, hyperinsulinemia, hypertrygliceridemia, insulin resistance, impaired glucose metabolism, erectile dysfunction, skin and connective tissue disorders, foot ulcerations and ulcerative colitis, endothelial dysfunction and impaired vascular compliance, hyper apo B lipoproteinemia, Alzheimer's, schizophrenia, impaired cognition, inflammatory bowel disease, ulcerative colitis, Crohn's disease, and irritable bowel syndrome, non-alcoholic steatohepatitis (NASH), or non-alcoholic fatty liver disease (NAFLD). portal hypertension, hepatic protein synthetic capability, hyperbilirubinemia, or encephalopathy. fatty liver, nonalcoholic fatty liver disease, nonalcoholic steatohepatitis, nonalcoholic steatohepatitis with liver fibrosis, nonalcoholic steatohepotitis with cirrhosis, or nonalcoholic steatohepatitis with cirrhosis and hepatocellular carcinoma.

In some embodiments, non-limiting examples of an APOC3-related disorder include: hyperlipidemia, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, angina pectoris, thrombosis, atherosclerosis, transient ischemic attacks, stroke, vascular restenosis, hyperglycemia, hyperinsulinemia, hypertrygliceridemia, insulin resistance, impaired glucose metabolism, erectile dysfunction, skin and connective tissue disorders, foot ulcerations and ulcerative colitis, endothelial dysfunction and impaired vascular compliance, hyper apo B lipoproteinemia, Alzheimer's, schizophrenia, impaired cognition, inflammatory bowel disease, ulcerative colitis, Crohn's disease, and irritable bowel syndrome, non-alcoholic steatohepatitis (NASH), or non-alcoholic fatty liver disease (NAFLD).

In some embodiments, non-limiting examples of an APOC3-related disorder include: fatty liver, nonalcoholic fatty liver disease, nonalcoholic steatohepatitis, nonalcoholic steatohepatitis with liver fibrosis, nonalcoholic steatohepotitis with cirrhosis, or nonalcoholic steatohepatitis with cirrhosis and hepatocellular carcinoma.

Treatment of an APOC3-Related Disorder

In some embodiments, the present disclosure pertains to an APOC3 oligonucleotide or a single-stranded RNAi agent to APOC3. Various single-stranded RNAi agents to APOC3 are disclosed herein.

APOC3 expression has reportedly been implicated in various disorders, including but not limited to: atherosclerosis or dyslipidemia, elevated triglyceride levels, elevated cholesterol levels, elevated free fatty acids, and diabetes. In some embodiments, the present disclosure pertains to a method of treating or ameliorating an APOC3-related disorder in a patient thereof, the method comprising the step of administering to the patient a therapeutically effective amount of an APOC3 oligonucleotide or a single-stranded RNAi agent to APOC3.

In some embodiments, the present disclosure pertains to a method for introducing an APOC3 oligonucleotide or a single-stranded RNAi agent that decreases APOC3 gene expression into a cell, the method comprising: contacting the cell with an APOC3 oligonucleotide or a single-stranded RNAi agent.

In some embodiments, the present disclosure pertains to a method for decreasing APOC3 gene expression in a mammal in need thereof, the method comprising: administering to the mammal a nucleic acid-lipid particle comprising an APOC3 oligonucleotide or a single-stranded RNAi agent to APOC3.

In some embodiments, the present disclosure pertains to a method for the in vivo delivery of an APOC3 oligonucleotide or a single-stranded RNAi agent that targets APOC3 gene expression, the method comprising: administering to a mammal an APOC3 oligonucleotide or a single-stranded RNAi agent to APOC3.

In some embodiments, the present disclosure pertains to a method for treating and/or ameliorating one or more symptoms associated with atherosclerosis or dyslipidemia in a mammal in need thereof, the method comprising: administering to the mammal a therapeutically effective amount of an APOC3 oligonucleotide or a single-stranded RNAi agent to APOC3.

In some embodiments, the present disclosure pertains to a method for reducing susceptibility to atherosclerosis or dyslipidemia in a mammal in need thereof, the method comprising: administering to the mammal a therapeutically effective amount of an APOC3 oligonucleotide or a single-stranded RNAi agent to APOC3.

In some embodiments, the present disclosure pertains to a method for preventing or delaying the onset of atherosclerosis or dyslipidemia in a mammal in need thereof, the method comprising: administering to the mammal a therapeutically effective amount of an APOC3 oligonucleotide or a single-stranded RNAi agent to APOC3.

In some embodiments, the present disclosure pertains to a method for lowering triglyceride levels in a mammal in need thereof, the method comprising: administering to the mammal a therapeutically effective amount of an APOC3 oligonucleotide or a single-stranded RNAi agent to APOC3.

In some embodiments, the method of the present disclosure is for the treatment of hyperlipidemia, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, angina pectoris, thrombosis, atherosclerosis, transient ischemic attacks, stroke, vascular restenosis, hyperglycemia, hyperinsulinemia, hypertrygliceridemia, elevated low density lipoprotein (LDL) cholesterol levels (hypercholesterolemia), insulin resistance, impaired glucose metabolism, erectile dysfunction, skin and connective tissue disorders, foot ulcerations and ulcerative colitis, endothelial dysfunction and impaired vascular compliance, hyper apo B lipoproteinemia, Alzheimer's, schizophrenia, impaired cognition, inflammatory bowel disease, ulcerative colitis, Crohn's disease, and irritable bowel syndrome, non-alcoholic steatohepatitis (NASH), or non-alcoholic fatty liver disease (NAFLD), in humans wherein the method comprises administering to a subject a therapeutically effective amount of an APOC3 oligonucleotide of the present disclosure.

In some embodiments, the method reduces portal hypertension, hepatic protein synthetic capability, hyperbilirubinemia, or encephalopathy wherein the method comprise administering to a subject a therapeutically effective amount of an APOC3 oligonucleotide of the present disclosure.

The present disclosure is also directed at a method for the treatment of reduction of at least one point in severity of nonalcoholic fatty liver disease or non-alcoholic steatohepatitis grading scoring systems, reduction of the level of serum markers of non-alcoholic steatohepatitis activity, reduction of non-alcoholic steatohepatitis disease activity or reduction in the medical consequences of non-alcoholic steatohepatitis in humans administering to a subject a therapeutically effective amount of an APOC3 oligonucleotide of the present disclosure. In some embodiments, the present disclosure pertains to a method for treating and/or ameliorating one or more symptoms associated with atherosclerosis or dyslipidemia in a mammal in need thereof, the method comprising: administering to the mammal a therapeutically effective amount of a nucleic acid-lipid particle comprising an APOC3 oligonucleotide or a single-stranded RNAi agent to APOC3.

In some embodiments, the present disclosure pertains to a method for reducing susceptibility to atherosclerosis or dyslipidemia in a mammal in need thereof, the method comprising: administering to the mammal a therapeutically effective amount of a nucleic acid-lipid particle comprising an APOC3 oligonucleotide or a single-stranded RNAi agent to APOC3.

In some embodiments, the present disclosure pertains to a method for preventing or delaying the onset of atherosclerosis or dyslipidemia in a mammal in need thereof, the method comprising: administering to the mammal a therapeutically effective amount of a nucleic acid-lipid particle comprising an APOC3 oligonucleotide or a single-stranded RNAi agent to APOC3.

In some embodiments, the present disclosure pertains to a method for lowering triglyceride levels in a mammal in need thereof, the method comprising: administering to the mammal a therapeutically effective amount of a nucleic acid-lipid particle comprising an APOC3 oligonucleotide or a single-stranded RNAi agent to APOC3.

In some embodiments, the present disclosure pertains to a method for lowering cholesterol levels in a mammal in need thereof, the method comprising: administering to the mammal a therapeutically effective amount of an APOC3 oligonucleotide or a single-stranded RNAi agent to APOC3.

In some embodiments, the present disclosure pertains to a method for lowering cholesterol levels in a mammal in need thereof, the method comprising: administering to the mammal a therapeutically effective amount of a nucleic acid-lipid particle comprising an APOC3 oligonucleotide or a single-stranded RNAi agent to APOC3.

In some embodiments, the present disclosure pertains to a method of inhibiting APOC3 expression in a cell, the method comprising: (a) contacting the cell with an APOC3 oligonucleotide or a single-stranded RNAi agent to APOC3; and (b) maintaining the cell produced in step (a) for a time sufficient to obtain degradation of the mRNA transcript of an APOC3 gene, thereby inhibiting expression of the APOC3 gene in the cell.

In some embodiments, APOC3 expression is inhibited by at least 30%.

In some embodiments, the present disclosure pertains to a method of treating a disorder mediated by APOC3 expression comprising administering to a human in need of such treatment a therapeutically effective amount of an APOC3 oligonucleotide or a single-stranded RNAi agent to APOC3.

In some embodiments, the disorder is elevated triglyceride levels.

In some embodiments, the disorder is triglyceride levels >150 mg/dL or >500 mg/dL.

In some embodiments, administration causes an increase in lipoprotein lipase and/or hepatic lipase activity.

In some embodiments, the present disclosure pertains to a method of improving peripheral insulin sensitivity comprising administering to a subject with diabetes an APOC3 oligonucleotide or a single-stranded RNAi agent to APOC3 thereby improving peripheral insulin sensitivity.

In some embodiments, the present disclosure pertains to a method of improving peripheral insulin sensitivity comprising administering to a subject with moderately controlled type II diabetes and administering an APOC3 oligonucleotide or a single-stranded RNAi agent to APOC3 thereby improving peripheral insulin sensitivity.

In some embodiments, the present disclosure pertains to a method of lowering free fatty acids comprising administering to a subject with diabetes and administering an APOC3 oligonucleotide or a single-stranded RNAi agent APOC3, thereby lowering free fatty acids.

In some embodiments, the present disclosure pertains to a method of lowering free fatty acids comprising administering to a subject with moderately controlled type II diabetes and administering an APOC3 oligonucleotide or a single-stranded RNAi agent to APOC3, thereby lowering free fatty acids.

In some embodiments, the present disclosure pertains to a method of reducing intramyocellular triglyceride deposition comprising administering to a subject with diabetes and administering an APOC3 oligonucleotide or a single-stranded RNAi agent to APOC3, thereby reducing intramyocellular triglyceride deposition.

In some embodiments, the present disclosure pertains to a method of reducing intramyocellular triglyceride deposition comprising administering to a subject with moderately controlled type II diabetes and administering an APOC3 oligonucleotide or a single-stranded RNAi agent to APOC3, thereby reducing intramyocellular triglyceride deposition.

In some embodiments, the present disclosure pertains to a method of improving a diabetes profile of a subject comprising administering to the subject an APOC3 oligonucleotide or a single-stranded RNAi agent to APOC3, wherein insulin sensitivity index, glucose disposal rate, glucose MCR, glucose metabolism:insulin ration is improved; wherein free fatty acids, triglycerides, non-HDL-C, VLDL-C, APOC3 containing VLDL, APOB and LDL-C are reduced and HDL-C is increased, thereby improving the diabetes profile of the subject.

In some embodiments, the subject is on a stable dose of metformin.

In some embodiments, the present disclosure pertains to a compound comprising an APOC3 oligonucleotide or a single-stranded RNAi agent to APOC3 for use in a subject to: improve peripheral insulin sensitivity; lower free fatty acids; reduce intramyocellular triglyceride deposition; improve a lipid profile; and/or improve a diabetic profile.

In some embodiments, a subject is administered a second agent (e.g., an additional therapeutic agent).

In some embodiments, the second agent is selected from an APOC3 lowering agent, cholesterol lowering agent, non-HDL lipid lowering agent, LDL lowering agent, TG lowering agent, cholesterol lowering agent, HDL raising agent, fish oil, niacin, fibrate, statin, DCCR (salt of diazoxide), glucose-lowering agent or anti-diabetic agents.

The oligonucleotides of the present disclosure can be administered alone or in combination with one or more additional therapeutic agents. By “administered in combination” or “combination therapy” it is meant that the oligonucleotide of the present disclosure and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect. Thus, the compositions and methods of prevention and treatment described herein include use of combination agents.

The combination agents are administered to a mammal in a therapeutically effective amount. By “therapeutically effective amount” it is meant an amount of an APOC3 oligonucleotide of the present disclosure that, when administered alone or in combination with an additional therapeutic agent to a mammal, is effective to treat the desired disease/condition (e.g., obesity, diabetes, and cardiovascular conditions).

In some embodiments, an oligonucleotides of the present disclosure may be administered in combination with an additional pharmaceutical agent selected from: an anti-inflammatory agent, an anti-diabetic agent, and a cholesterol/lipid modulating agent.

In some embodiments, an oligonucleotides of the present disclosure may be administered in combination with an additional pharmaceutical agent selected from: an acetyl-CoA carboxylase-(ACC) inhibitor, a diacylglycerol O-acyltransferase 1 (DGAT-1) inhibitor, a diacylglycerol O-acyltransferase 2 (DGAT-2) inhibitor, monoacylglycerol O-acyltransferase inhibitors, a phosphodiesterase (PDE)-10 inhibitor, an AMPK activator, a sulfonylurea, a meglitinide, an α-amylase inhibitor, an α-glucoside hydrolase inhibitor, an α-glucosidase inhibitor, a PPARγ agonist, a PPAR α/γ agonist, a biguanide, a glucagon-like peptide 1 (GLP-1) modulator, liraglutide, albiglutide, exenatide, albiglutide, lixisenatide, dulaglutide, semaglutide, a protein tyrosine phosphatase-1B (PTP-1B) inhibitor, SIRT-1 activator, a dipeptidyl peptidase IV (DPP-IV) inhibitor, an insulin secreatagogue, a fatty acid oxidation inhibitor, an A2 antagonist, a c-jun amino-terminal kinase (JNK) inhibitor, glucokinase activators (GKa), insulin, an insulin mimetic, a glycogen phosphorylase inhibitor, a VPAC2 receptor agonist, SGLT2 inhibitors, a glucagon receptor modulator, GPR119 modulators, FGF21 derivatives or analogs, TGRS receptor modulators, GPBAR1 receptor modulators, GPR40 agonists, GPR120 modulators, high affinity nicotinic acid receptor (HM74A) activators, SGLT1 inhibitors, inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g. PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostain receptors, inhibitors or modulators of PDHK2 or PDHK4, inhibitors of MAP4K4, modulators of IL1 family including IL1beta, HMG-CoA reductase inhibitors, squalene synthetase inhibitors, fibrates, bile acid sequestrants, ACAT inhibitors, MTP inhibitors, lipooxygenase inhibitors, cholesterol absorption inhibitors, PCSK9 modulators, cholesteryl ester transfer protein inhibitors and modulators of RXRalpha.

In some embodiments, an oligonucleotides of the present disclosure may be administered in combination with an additional pharmaceutical agent selected from: cysteamine or a pharmaceutically acceptable salt thereof, cystamine or a pharmaceutically acceptable salt thereof, an anti-oxidant compound, lecithin, vitamin B complex, a bile salt preparations, an antagonists of Cannabinoid-1 (CB1) receptor, an inverse agonists of Cannabinoid-1 (CB1) receptor, a peroxisome proliferator-activated receptor) activity regulators, a benzothiazepine or benzothiepine compound, an RNA antisense construct to inhibit protein tyrosine phosphatase PTPRU, a heteroatom-linked substituted piperidine and derivatives thereof, an azacyclopentane derivative capable of inhibiting stearoyl-coenzyme alpha delta-9 desaturase, acylamide compound having secretagogue or inducer activity of adiponectin, a quaternary ammonium compound, Glatiramer acetate, pentraxin proteins, a HMG-CoA reductase inhibitor, n-acetyl cysteine, isoflavone compound, a macrolide antibiotic, a galectin inhibitor, an antibody, or any combination of thereof.

In some embodiments, an oligonucleotide of the present disclosure may be administered in combination with a treatment of non-alcoholic steatohepatitis (NASH) and/or non-alcoholic fatty liver disease (NAFLD) (i.e., anti-NASH and anti-NAFLD agents), such as Orlistat, TZDs and other insulin sensitizing agents, FGF21 analogs, Metformin, Omega-3-acid ethyl esters (e.g. Lovaza), Fibrates, HMG CoA-reductase Inhibitors, Ezitimbe, Probucol, Ursodeoxycholic acid, TGRS agonists, FXR agonists, Vitamin E, Betaine, Pentoxifylline, CB1 antagonists, Carnitine, N-acetylcysteine, Reduced glutathione, lorcaserin, the combination of naltrexone with buproprion, SGLT2 Inhibitors, Phentermine, Topiramate, Incretin (GLP and GIP) analogs and Angiotensin-receptor blockers. Preferred agents for the treatment of non-alcoholic steatohepatitis (NASH) and/or non-alcoholic fatty liver disease (NAFLD) (i.e., anti-NASH and anti-NAFLD agents) are an acetyl-CoA carboxylase (ACC) inhibitor, a ketohexokinase (KHK) inhibitor, a GLP-1 receptor agonist, an FXR agonist, a CB1 antagonist, an ASK1 inhibitor, an inhibitor of CCR2 and/or CCR5, a PNPLA3 inhibitor, a DGAT1 inhibitor, a DGAT2 inhibitor, an FGF21 analog, an FGF19 analog, an SGLT2 inhibitor, a PPAR agonist, an AMPK activator, an SCD1 inhibitor or an MPO inhibitor. A commonly assigned patent application PCT/IB2017/057577 filed Dec. 1, 2017.is directed to GLP-1 receptor agonists. Most preferred are a FXR agonist, an apoptosis signal-regulating kinase 1 (ASK1) inhibitor, a PPAR agonist, a GLP-1 receptor agonist, a SGLT inhibitor, a an ACC inhibitor and a KHK inhibitor.

In some embodiments, an oligonucleotide of the present disclosure may be administered in combination with an anti-diabetic agent including any of: insulin, metfomin, DPPIV inhibitors (e.g., sitagliptin), GLP-1 receptor agonists, analogues and mimetics, SGLT1 and SGLT2 inhibitors (e.g., ertuglifozin)). Preferred agents are metaformin, sitagliptin and ertuglifozin. Suitable anti-diabetic agents include an acetyl-CoA carboxylase- (ACC) inhibitor such as those described in WO2009144554, WO2003072197, WO2009144555 and WO2008065508, a diacylglycerol O-acyltransferase 1 (DGAT-1) inhibitor, such as those described in WO09016462 or WO2010086820, AZD7687 or LCQ908, a diacylglycerol O-acyltransferase 2 (DGAT-2) inhibitor, such as those described in WO2015/140658, monoacylglycerol O-acyltransferase inhibitors, a phosphodiesterase (PDE)-10 inhibitor, an AMPK activator, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone and rosiglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) receptor agonist (e.g., exendin-3 and exendin-4), liraglutide, albiglutide, exenatide (Byetta®), albiglutide, lixisenatide, dulaglutide, semaglutide, NN-9924, TTP-054. TTP-273, a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 activator (e.g., resveratrol, GSK2245840 or GSK184072), a dipeptidyl peptidease IV (DPP-IV) inhibitor (e.g., those in WO2005116014, sitagliptin, vildagliptin, alogliptin, dutogliptin, linagliptin and saxagliptin), an insulin secreatagogue, a fatty acid oxidation inhibitor, an A2 antagonist, a c-jun amino-terminal kinase (JNK) inhibitor, glucokinase activators (GKa) such as those described in WO2010103437, WO2010103438, WO2010013161, WO2007122482, TTP-399, TTP-355, TTP-547, AZD1656, ARRY403, MK-0599, TAK-329, AZD5658 or GKM-001, insulin, an insulin mimetic, a glycogen phosphorylase inhibitor (e.g. GSK1362885), a VPAC2 receptor agonist, SGLT2 inhibitors, such as those described in E. C. Chao et al. Nature Reviews Drug Discovery 9, 551-559 (July 2010) including dapagliflozin, canagliflozin, empagliflozin, tofogliflozin (CSG452), Ertugliflozin, ASP-1941, THR1474, TS-071, ISIS388626 and LX4211 as well as those in WO2010023594, a glucagon receptor modulator such as those described in Demong, D. E. et al. Annual Reports in Medicinal Chemistry 2008, 43, 119-137, GPR119 modulators, particularly agonists, such as those described in WO2010140092, WO2010128425, WO2010128414, WO2010106457, Jones, R. M. et al. in Medicinal Chemistry 2009, 44, 149-170 (e.g. MBX-2982, GSK1292263, APD597 and PSN821), FGF21 derivatives or analogs such as those described in Kharitonenkov, A. et al. et al., Current Opinion in Investigational Drugs 2009, 10(4)359-364, TGRS (also termed GPBAR1) receptor modulators, particularly agonists, such as those described in Zhong, M., Current Topics in Medicinal Chemistry, 2010, 10(4), 386-396 and INT777, GPR40 agonists, such as those described in Medina, J. C., Annual Reports in Medicinal Chemistry, 2008, 43, 75-85, including but not limited to TAK-875, GPR120 modulators, particularly agonists, high affinity nicotinic acid receptor (HM74A) activators, and SGLT1 inhibitors, such as GSK1614235. A further representative listing of anti-diabetic agents that can be combined with the oligonucleotides of the present disclosure can be found, for example, at page 28, line 35 through page 30, line 19 of WO2011005611. Preferred anti-diabetic agents are metformin and DPP-IV inhibitors (e.g., sitagliptin, vildagliptin, alogliptin, dutogliptin, linagliptin and saxagliptin). Other antidiabetic agents could include inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g. PKCα, PKCβ, PKCγ), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostain receptors (e.g. SSTR1, SSTR2, SSTR3 and SSTR5), inhibitors or modulators of PDHK2 or PDHK4, inhibitors of MAP4K4, modulators of IL1 family including IL1beta, modulators of RXRalpha. In addition suitable anti-diabetic agents include mechanisms listed by Carpino, P. A., Goodwin, B. Expert Opin. Ther. Pat, 2010, 20(12), 1627-51.

Suitable anti-obesity agents include 11P-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β3 adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY3-36 (including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y., and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, the combination of naltrexone with buproprion and the like.

Preferred anti-obesity agents for use in the combination aspects of the present disclosure include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY3-36 (as used herein “PYY3-36” includes analogs, such as peglated PYY3-36 e.g., those described in US Publication 2006/0178501), opioid antagonists (e.g., naltrexone), the combination of naltrexone with buproprion, oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3), phentermine and topiramate (trade name: Qsymia), and sibutramine. Preferably, oligonucleotides of the present disclosure and combination therapies are administered in conjunction with exercise and a sensible diet.

Those skilled in the art will recognize that oligonucleotides of the present disclosure may also be used in conjunction with cardiovascular or cerebrovascular treatments as described in the paragraphs below. Oligonucleotides of the present disclosure may also be used with cardiovascular therapies including PCI, stenting, drug eluting stents, stem cell therapy and medical devices such as implanted pacemakers, defibrillators, or cardiac resynchronization therapy.

An oligonucleotide of the present disclosure may be used in combination with any of: cholesterol modulating agents (including cholesterol lowering agents) such as a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, an ion-exchange resin, an antioxidant, an ACAT inhibitor or a bile acid sequestrant or an agent such as mipomersen.

Examples of suitable cholesterol/lipid lowering agents and lipid profile therapies include: HMG-CoA reductase inhibitors (e.g., pravastatin, lovastatin, atorvastatin, simvastatin, fluvastatin, NK-104 (a.k.a. itavastatin, or nisvastatin or nisbastatin) and ZD-4522 (a.k.a. rosuvastatin, or atavastatin or visastatin); squalene synthetase inhibitors; fibrates; bile acid sequestrants (such as questran); ACAT inhibitors; MTP inhibitors; lipooxygenase inhibitors; cholesterol absorption inhibitors; and cholesteryl ester transfer protein inhibitors. Other atherosclerotic agents include PCSK9 modulators.

Administration of an APOC3 Oligonucleotide or a Single-Stranded RNAi Agent

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides are administered with any vehicle, in any dosing regiment, and in any manner described herein or known in the art.

In some embodiments, a provided oligonucleotide composition is administered at a dose and/or frequency lower than that of an otherwise comparable reference oligonucleotide composition with comparable effect in improving the knockdown of a target transcript. In some embodiments, a stereocontrolled oligonucleotide composition is administered at a dose and/or frequency lower than that of an otherwise comparable stereorandom reference oligonucleotide composition with comparable effect in improving the knockdown of the target transcript.

In some embodiments, the present disclosure recognizes that properties, e.g., improved single-stranded RNA interference activity, etc. of oligonucleotides and compositions thereof can be optimized by chemical modifications and/or stereochemistry. In some embodiments, the present disclosure provides methods for optimizing oligonucleotide properties through chemical modifications and stereochemistry.

By controlling of chemical modifications and/or stereochemistry, the present disclosure provides improved oligonucleotide compositions and methods. In some embodiments, provided oligonucleotides capable of directing single-stranded RNA interference comprise chemical modifications. In some embodiments, provided oligonucleotides capable of directing single-stranded RNA interference comprise base modifications, sugar modifications, internucleotidic linkage modifications, or any combinations thereof. In some embodiments, provided oligonucleotides capable of directing single-stranded RNA interference comprise base modifications. In some embodiments, provided oligonucleotides capable of directing single-stranded RNA interference comprise sugar modifications. In some embodiments, provided oligonucleotides comprises 2′-modifications on the sugar moieties. In some embodiments, provided oligonucleotides comprises one or more modified internucleotidic linkages and one or more natural phosphate linkages. A natural phosphate linkage can be incorporated into various locations of an APOC3 oligonucleotide. In some embodiments, a natural phosphate linkage is incorporated into the 5′-end region. In some embodiments, a natural phosphate linkage is incorporated into the middle of an APOC3 oligonucleotide. In some embodiments, the present disclosure provides a method comprising administering a composition comprising a first plurality of oligonucleotides, which composition displays improved delivery as compared with a reference composition comprising a plurality of oligonucleotides, each of which also has the common base sequence but which differs structurally from the oligonucleotides of the first plurality in that:

individual oligonucleotides within the reference plurality differ from one another in stereochemical structure; and/or

at least some oligonucleotides within the reference plurality have a structure different from a structure represented by the plurality of oligonucleotides of the composition.

In some embodiments, the present disclosure provides a method of administering an APOC3 oligonucleotide composition comprising a first plurality of oligonucleotides capable of directing single-stranded RNA interference and having a common nucleotide sequence, the improvement that comprises:

administering an APOC3 oligonucleotide comprising a first plurality of oligonucleotides that is characterized by improved delivery relative to a reference oligonucleotide composition of the same common nucleotide sequence.

In some embodiments, provided oligonucleotides, compositions and methods provide improved systemic delivery. In some embodiments, provided oligonucleotides, compositions and methods provide improved cytoplasmatic delivery. In some embodiments, improved delivery is to a population of cells. In some embodiments, improved delivery is to a tissue. In some embodiments, improved delivery is to an organ. In some embodiments, improved delivery is to an organism. Example structural elements (e.g., chemical modifications, stereochemistry, combinations thereof, etc.), oligonucleotides, compositions and methods that provide improved delivery are extensively described in this disclosure.

In some embodiments, the present disclosure provides a method of identifying and/or characterizing an APOC3 oligonucleotide composition, the method comprising steps of:

providing at least one composition comprising a first plurality of oligonucleotides; and

assessing delivery relative to a reference composition.

In some embodiments, the present disclosure provides a method of identifying and/or characterizing an APOC3 oligonucleotide composition, the method comprising steps of:

providing at least one composition comprising a first plurality of oligonucleotides; and

assessing cellular uptake relative to a reference composition.

In some embodiments, properties of a provided oligonucleotide compositions are compared to a reference oligonucleotide composition. In some embodiments, a reference oligonucleotide composition comprises a reference plurality of oligonucleotides.

In some embodiments, a reference oligonucleotide composition is a stereorandom oligonucleotide composition. In some embodiments, a reference oligonucleotide composition is a stereorandom composition of oligonucleotides of which all internucleotidic linkages are phosphorothioate. In some embodiments, a reference oligonucleotide composition is a DNA oligonucleotide composition with all phosphate linkages.

In some embodiments, a reference composition is a composition of oligonucleotides having the same base sequence and the same chemical modifications. In some embodiments, a reference composition is a composition of oligonucleotides having the same base sequence and the same pattern of chemical modifications. In some embodiments, a reference composition is a chirally un-controlled (or stereorandom) composition of oligonucleotides having the same base sequence and chemical modifications.

In some embodiments, a reference composition is a composition of oligonucleotides having the same base sequence but different chemical modifications. In some embodiments, a reference composition is a composition of oligonucleotides having the same base sequence, base modifications, internucleotidic linkage modifications but different sugar modifications. In some embodiments, a reference composition has fewer 2′-modified sugar modifications. In some embodiments, a reference composition is a composition of oligonucleotides having the same base sequence, base modifications, sugar modifications but different internucleotidic linkage modifications. In some embodiments, a reference composition has more internucleotidic linkage modifications. In some embodiments, a reference composition has fewer natural phosphate linkages. In some embodiments, a reference composition comprising oligonucleotides having no natural phosphate linkages.

In some embodiments, a reference composition is a composition comprising a reference plurality of oligonucleotides wherein individual oligonucleotides within the reference plurality differ from one another in stereochemical structure. In some embodiments, a reference composition is a composition comprising a reference plurality of oligonucleotides, wherein at least some oligonucleotides within the reference plurality have a structure different from a structure represented by a plurality of oligonucleotides of a composition compared to the reference composition.

In some embodiments, a reference oligonucleotide composition comprises a reference plurality of oligonucleotides capable of directing single-stranded RNA interference and having the same common nucleotide sequence but lacking at least one of the one or more modified sugar moieties in oligonucleotides of the oligonucleotide composition compared to the reference composition. In some embodiments, a reference oligonucleotide composition comprises a reference plurality of oligonucleotides capable of directing single-stranded RNA interference and having the same common nucleotide sequence but have no modified sugar moieties. In some embodiments, a reference oligonucleotide composition comprises a reference plurality of oligonucleotides capable of directing single-stranded RNA interference and having the same common nucleotide sequence but do not comprise natural phosphate linkages. In some embodiments, a reference composition is an APOC3 oligonucleotide or a single-stranded RNAi agent of oligonucleotides having the same chemical modification patterns. In some embodiments, a reference composition is an APOC3 oligonucleotide or a single-stranded RNAi agent of another stereoisomer.

In some embodiments, a reference oligonucleotide composition of a provided oligonucleotide composition is a comparable composition absence of the lipids in the provided composition. In some embodiments, a reference oligonucleotide composition is a stereorandom oligonucleotide composition. In some embodiments, a reference oligonucleotide composition is a stereorandom composition of oligonucleotides of which all internucleotidic linkages are phosphorothioate. In some embodiments, a reference oligonucleotide composition is a DNA oligonucleotide composition with all phosphate linkages. In some embodiments, a reference composition is a composition of oligonucleotides having the same base sequence and the same chemical modifications. In some embodiments, a reference composition is a composition of oligonucleotides having the same base sequence and the same pattern of chemical modifications. In some embodiments, a reference composition is a chirally un-controlled (or stereorandom) composition of oligonucleotides having the same base sequence and chemical modifications. In some embodiments, a reference composition is a composition of oligonucleotides having the same base sequence but different chemical modifications. In some embodiments, a reference composition is a composition of oligonucleotides having the same base sequence, base modifications, internucleotidic linkage modifications but different sugar modifications. In some embodiments, a reference composition has fewer 2′-modified sugar modifications. In some embodiments, a reference composition is a composition of oligonucleotides having the same base sequence, base modifications, sugar modifications but different internucleotidic linkage modifications. In some embodiments, a reference composition has more internucleotidic linkage modifications. In some embodiments, a reference composition has fewer natural phosphate linkages. In some embodiments, a reference composition comprising oligonucleotides having no natural phosphate linkages. In some embodiments, a reference composition is a composition comprising a reference plurality of oligonucleotides wherein individual oligonucleotides within the reference plurality differ from one another in stereochemical structure. In some embodiments, a reference composition is a composition comprising a reference plurality of oligonucleotides, wherein at least some oligonucleotides within the reference plurality have a structure different from a structure represented by a plurality of oligonucleotides of a composition compared to the reference composition. In some embodiments, a reference oligonucleotide composition comprises a reference plurality of oligonucleotides capable of directing single-stranded RNA interference and having the same common nucleotide sequence but lacking at least one of the one or more modified sugar moieties in oligonucleotides of the oligonucleotide composition compared to the reference composition. In some embodiments, a reference oligonucleotide composition comprises a reference plurality of oligonucleotides capable of directing single-stranded RNA interference and having the same common nucleotide sequence but have no modified sugar moieties. In some embodiments, a reference oligonucleotide composition comprises a reference plurality of oligonucleotides capable of directing single-stranded RNA interference and having the same common nucleotide sequence but do not comprise natural phosphate linkages. In some embodiments, a reference composition is an APOC3 oligonucleotide or a single-stranded RNAi agent of oligonucleotides having the same chemical modification patterns. In some embodiments, a reference composition is an APOC3 oligonucleotide or a single-stranded RNAi agent of another stereoisomer.

In some embodiments, oligonucleotides of the first plurality comprise one or more structural elements (e.g., modifications, stereochemistry, patterns, etc.) that oligonucleotides of the reference plurality do not all have. Such structural elements can be any one described in this disclosure.

In some embodiments, oligonucleotides of the first plurality comprise more phosphorothioate linkages tha APOC3 oligonucleotides of the reference composition. In some embodiments, oligonucleotides of the first plurality comprise more phosphorothioate linkages tha APOC3 oligonucleotides of the reference composition at the 5′-end region. In some embodiments, oligonucleotides of the first plurality comprise more phosphorothioate linkages tha APOC3 oligonucleotides of the reference composition at the 3′-end region. In some embodiments, oligonucleotides of the first plurality comprise more Sp chiral internucleotidic linkages tha APOC3 oligonucleotides of the reference composition. In some embodiments, oligonucleotides of the first plurality comprise more Sp phosphorothioate linkages tha APOC3 oligonucleotides of the reference composition. In some embodiments, oligonucleotides of the first plurality comprise more Sp phosphorothioate linkages tha APOC3 oligonucleotides of the reference composition at the 5′-end region. In some embodiments, oligonucleotides of the first plurality comprise more Sp phosphorothioate linkages tha APOC3 oligonucleotides of the reference composition at the 3′-end region. In some embodiments, oligonucleotides of the first plurality comprise more modified bases tha APOC3 oligonucleotides of the reference composition. In some embodiments, oligonucleotides of the first plurality comprise more methylated bases tha APOC3 oligonucleotides of the reference composition. In some embodiments, oligonucleotides of the first plurality comprise more methylated bases tha APOC3 oligonucleotides of the reference composition at the 5′-end region. In some embodiments, oligonucleotides of the first plurality comprise more methylated bases tha APOC3 oligonucleotides of the reference composition at the 3′-end region. In some embodiments, oligonucleotides of the first plurality comprise fewer 2′-MOE modifications tha APOC3 oligonucleotides of the reference composition. In some embodiments, oligonucleotides of the first plurality comprise fewer 2′-MOE modifications tha APOC3 oligonucleotides of the reference composition. In some embodiments, oligonucleotides of the first plurality comprise fewer 2′-MOE modifications tha APOC3 oligonucleotides of the reference composition at the 5′-end region. In some embodiments, oligonucleotides of the first plurality comprise fewer 2′-MOE modifications tha APOC3 oligonucleotides of the reference composition at the 3′-end region. In some embodiments, individual oligonucleotides within the reference plurality differ from one another in stereochemical structure. In some embodiments, at least some oligonucleotides within the reference plurality have a structure different from a structure represented by the plurality of oligonucleotides of the composition. In some embodiments, the reference composition is a substantially racemic preparation of oligonucleotides that share the base sequence. In some embodiments, the reference composition is an APOC3 oligonucleotide or a single-stranded RNAi agent of another oligonucleotide type. In some embodiments, oligonucleotides of the reference composition comprise more phosphorothioate linkages. In some embodiments, oligonucleotides of the reference composition comprise only phosphorothioate linkages. In some embodiments, oligonucleotides of the reference composition comprise fewer modified sugar moieties. In some embodiments, oligonucleotides of the reference composition comprise fewer modified sugar moieties, wherein the modification is 2′-OR¹. In some embodiments, oligonucleotides of the reference composition comprise more modified sugar moieties. In some embodiments, oligonucleotides of the reference composition comprise more modified sugar moieties, the modification is 2′-OR¹. In some embodiments, oligonucleotides of the reference composition comprise fewer phosphorothioate linkages. In some embodiments, oligonucleotides of the reference composition comprise fewer methylated bases. In some embodiments, oligonucleotides of the reference composition comprise more 2′-MOE modifications. In some embodiments, oligonucleotides of the reference composition comprise fewer natural phosphate linkages. In some embodiments, oligonucleotides of the reference composition comprise fewer natural phosphate linkages at the 5′- and/or 3′-end region In some embodiments, oligonucleotides of a provided composition comprise fewer 2′-MOE modifications tha APOC3 oligonucleotides of the reference composition. In some embodiments, oligonucleotides of a provided composition comprise fewer 2′-MOE modifications tha APOC3 oligonucleotides of the reference composition. In some embodiments, oligonucleotides of a provided composition comprise fewer 2′-MOE modifications tha APOC3 oligonucleotides of the reference composition at the 5′-end region. In some embodiments, oligonucleotides of a provided composition comprise fewer 2′-MOE modifications tha APOC3 oligonucleotides of the reference composition at the 3′-end region. In some embodiments, individual oligonucleotides within the reference plurality differ from one another in stereochemical structure. In some embodiments, at least some oligonucleotides within the reference plurality have a structure different from a structure represented by the plurality of oligonucleotides of the composition. In some embodiments, the reference composition is a substantially racemic preparation of oligonucleotides that share the base sequence. In some embodiments, the reference composition is an APOC3 oligonucleotide or a single-stranded RNAi agent of another oligonucleotide type. In some embodiments, oligonucleotides of the reference composition comprise more phosphorothioate linkages. In some embodiments, oligonucleotides of the reference composition comprise only phosphorothioate linkages. In some embodiments, oligonucleotides of the reference composition comprise fewer modified sugar moieties. In some embodiments, oligonucleotides of the reference composition comprise fewer modified sugar moieties, wherein the modification is 2′-OR¹. In some embodiments, oligonucleotides of the reference composition comprise more modified sugar moieties. In some embodiments, oligonucleotides of the reference composition comprise more modified sugar moieties, the modification is 2′-OR¹. In some embodiments, oligonucleotides of the reference composition comprise fewer phosphorothioate linkages. In some embodiments, oligonucleotides of the reference composition comprise fewer methylated bases. In some embodiments, oligonucleotides of the reference composition comprise more 2′-MOE modifications. In some embodiments, oligonucleotides of the reference composition comprise fewer natural phosphate linkages. In some embodiments, oligonucleotides of the reference composition comprise fewer natural phosphate linkages at the 5′- and/or 3′-end region. In some embodiments, oligonucleotides of a reference plurality comprise fewer nucleotidic units comprising —F. In some embodiments, oligonucleotides of a reference plurality comprise fewer 2′-F modified sugar moieties. In some embodiments, oligonucleotides of a reference plurality comprise fewer chirally controlled modified internucleotidic linkages.

In some embodiments, provided chirally controlled oligonucleotide compositions comprises oligonucleotides of one oligonucleotide type. In some embodiments, provided chirally controlled oligonucleotide compositions comprises oligonucleotides of only one oligonucleotide type. In some embodiments, provided chirally controlled oligonucleotide compositions has oligonucleotides of only one oligonucleotide type. In some embodiments, provided chirally controlled oligonucleotide compositions comprises oligonucleotides of two or more oligonucleotide types. In some embodiments, using such compositions, provided methods can target more than one target. In some embodiments, an APOC3 oligonucleotide or a single-stranded RNAi agent comprising two or more oligonucleotide types targets two or more targets. In some embodiments, an APOC3 oligonucleotide or a single-stranded RNAi agent comprising two or more oligonucleotide types targets two or more mismatches. In some embodiments, a single oligonucleotide type targets two or more targets, e.g., mutations. In some embodiments, a target region of oligonucleotides of one oligonucleotide type comprises two or more “target sites” such as two mutations or SNPs.

In some embodiments, oligonucleotides in a provided chirally controlled oligonucleotide composition optionally comprise modified bases or sugars. In some embodiments, a provided chirally controlled oligonucleotide composition does not have any modified bases or sugars. In some embodiments, a provided chirally controlled oligonucleotide composition does not have any modified bases. In some embodiments, oligonucleotides in a provided chirally controlled oligonucleotide composition comprise modified bases and sugars. In some embodiments, oligonucleotides in a provided chirally controlled oligonucleotide composition comprise a modified base. In some embodiments, oligonucleotides in a provided chirally controlled oligonucleotide composition comprise a modified sugar. Modified bases and sugars for oligonucleotides are widely known in the art, including but not limited in those described in the present disclosure. In some embodiments, a modified base is 5-mC. In some embodiments, a modified sugar is a 2′-modified sugar. Suitable 2′-modification of oligonucleotide sugars are widely known by a person having ordinary skill in the art. In some embodiments, 2′-modifications include but are not limited to 2′-OR¹, wherein R¹ is not hydrogen. In some embodiments, a 2′-modification is 2′-OR¹, wherein R¹ is optionally substituted C₁₋₆ aliphatic. In some embodiments, a 2′-modification is 2′-MOE. In some embodiments, a modification is 2′-halogen. In some embodiments, a modification is 2′-F. In some embodiments, modified bases or sugars may further enhance activity, stability and/or selectivity of a chirally controlled oligonucleotide composition, whose common pattern of backbone chiral centers provides unexpected activity, stability and/or selectivity.

In some embodiments, a provided chirally controlled oligonucleotide composition does not have any modified sugars. In some embodiments, a provided chirally controlled oligonucleotide composition does not have any 2′-modified sugars. In some embodiments, the present disclosure surprising found that by using chirally controlled oligonucleotide compositions, modified sugars are not needed for stability, activity, and/or control of cleavage patterns. Furthermore, in some embodiments, the present disclosure surprisingly found that chirally controlled oligonucleotide compositions of oligonucleotides without modified sugars deliver better properties in terms of stability, activity, turn-over and/or control of cleavage patterns. For example, in some embodiments, it is surprising found that chirally controlled oligonucleotide compositions of oligonucleotides having no modified sugars dissociates much faster from cleavage products and provide significantly increased turn-over than compositions of oligonucleotides with modified sugars.

As discussed in detail herein, the present disclosure provides, among other things, a chirally controlled oligonucleotide composition, meaning that the composition contains a plurality of oligonucleotides of at least one type. Each oligonucleotide molecule of a particular “type” is comprised of preselected (e.g., predetermined) structural elements with respect to: (1) base sequence; (2) pattern of backbone linkages; (3) pattern of backbone chiral centers; and (4) pattern of backbone P-modification moieties. In some embodiments, provided oligonucleotide compositions contain oligonucleotides that are prepared in a single synthesis process. In some embodiments, provided compositions contain oligonucleotides having more than one chiral configuration within a single oligonucleotide molecule (e.g., where different residues along the oligonucleotide have different stereochemistry); in some such embodiments, such oligonucleotides may be obtained in a single synthesis process, without the need for secondary conjugation steps to generate individual oligonucleotide molecules with more than one chiral configuration.

Oligonucleotide compositions as provided herein can be used as single-stranded RNAi agents. In addition, oligonucleotide compositions as provided herein can be used as reagents for research and/or diagnostic purposes. One of ordinary skill in the art will readily recognize that the present disclosure disclosure herein is not limited to particular use but is applicable to any situations where the use of synthetic oligonucleotides is desirable. Among other things, provided compositions are useful in a variety of therapeutic, diagnostic, agricultural, and/or research applications.

In some embodiments, provided oligonucleotide compositions comprise oligonucleotides and/or residues thereof that include one or more structural modifications as described in detail herein. In some embodiments, provided oligonucleotide compositions comprise oligonucleotides that contain one or more modified nucleotides. In some embodiments, provided oligonucleotide compositions comprise oligonucleotides that contain one or more artificial nucleic acids or residues, including but not limited to: peptide nucleic acids (PNA), Morpholino and locked nucleic acids (LNA), glycon nucleic acids (GNA), threose nucleic acids (TNA), Xeno nucleic acids (XNA), manitol nucleic acid (MNA), anitol nucleic acid (ANA), and F-HNA, and any combination thereof. In some embodiments, a provided oligonucleotide comprises a Morpholino as described in Braasch et al. 2002 Biochem. 41: 4503-4510, or U.S. Pat. Nos. 5,698,685; 5,166,315; 5,185,444; or 5,034,506. In some embodiments, a provided oligonucleotide comprises a F-HNA as described in U.S. Pat. Nos. 8,088,904; 8,440,803; or 8,796,437; or in WO 2017/011276. Various modified nucleotides, including modified sugars are described in, for example, WO 2016/154096 and WO 2016/141236.

In any of the embodiments, the disclosure is useful for oligonucleotide-based modulation of gene expression, immune response, etc. Accordingly, stereo-defined, oligonucleotide compositions of the disclosure, which contain oligonucleotides of predetermined type (i.e., which are chirally controlled, and optionally chirally pure), can be used in lieu of conventional stereo-random or chirally impure counterparts. In some embodiments, provided compositions show enhanced intended effects and/or reduced unwanted side effects. Certain embodiments of biological and clinical/therapeutic applications of the disclosure are discussed explicitly below.

Various dosing regimens can be utilized to administer provided chirally controlled oligonucleotide compositions. In some embodiments, multiple unit doses are administered, separated by periods of time. In some embodiments, a given composition has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second (or subsequent) dose amount that is same as or different from the first dose (or another prior dose) amount. In some embodiments, a dosing regimen comprises administering at least one unit dose for at least one day. In some embodiments, a dosing regimen comprises administering more than one dose over a time period of at least one day, and sometimes more than one day. In some embodiments, a dosing regimen comprises administering multiple doses over a time period of at least week. In some embodiments, the time period is at least 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35, 36, 37, 38, 39, 40 or more (e.g., about 45, 50, 55, 60, 65, 70, 75, 80, 85, 90, 95, 100 or more) weeks. In some embodiments, a dosing regimen comprises administering one dose per week for more than one week. In some embodiments, a dosing regimen comprises administering one dose per week for 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35, 36, 37, 38, 39, 40 or more (e.g., about 45, 50, 55, 60, 65, 70, 75, 80, 85, 90, 95, 100 or more) weeks. In some embodiments, a dosing regimen comprises administering one dose every two weeks for more than two week period. In some embodiments, a dosing regimen comprises administering one dose every two weeks over a time period of 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 33, 34, 35, 36, 37, 38, 39, 40 or more (e.g., about 45, 50, 55, 60, 65, 70, 75, 80, 85, 90, 95, 100 or more) weeks. In some embodiments, a dosing regimen comprises administering one dose per month for one month. In some embodiments, a dosing regimen comprises administering one dose per month for more than one month. In some embodiments, a dosing regimen comprises administering one dose per month for 2, 3, 4, 5, 6, 7, 8, 9, 10, 11, 12, or more months. In some embodiments, a dosing regimen comprises administering one dose per week for about 10 weeks. In some embodiments, a dosing regimen comprises administering one dose per week for about 20 weeks. In some embodiments, a dosing regimen comprises administering one dose per week for about 30 weeks. In some embodiments, a dosing regimen comprises administering one dose per week for 26 weeks. In some embodiments, an APOC3 oligonucleotide or a single-stranded RNAi agent is administered according to a dosing regimen that differs from that utilized for a chirally uncontrolled (e.g., stereorandom) oligonucleotide composition of the same sequence, and/or of a different chirally controlled oligonucleotide composition of the same sequence. In some embodiments, an APOC3 oligonucleotide or a single-stranded RNAi agent is administered according to a dosing regimen that is reduced as compared with that of a chirally uncontrolled (e.g., stereorandom) oligonucleotide composition of the same sequence in that it achieves a lower level of total exposure over a given unit of time, involves one or more lower unit doses, and/or includes a smaller number of doses over a given unit of time. In some embodiments, an APOC3 oligonucleotide or a single-stranded RNAi agent is administered according to a dosing regimen that extends for a longer period of time than does that of a chirally uncontrolled (e.g., stereorandom) oligonucleotide composition of the same sequence Without wishing to be limited by theory, Applicant notes that in some embodiments, the shorter dosing regimen, and/or longer time periods between doses, may be due to the improved stability, bioavailability, and/or efficacy of a chirally controlled oligonucleotide composition. In some embodiments, an APOC3 oligonucleotide or a single-stranded RNAi agent has a longer dosing regimen compared to the corresponding chirally uncontrolled oligonucleotide composition. In some embodiments, an APOC3 oligonucleotide or a single-stranded RNAi agent has a shorter time period between at least two doses compared to the corresponding chirally uncontrolled oligonucleotide composition. Without wishing to be limited by theory, Applicant notes that in some embodiments longer dosing regimen, and/or shorter time periods between doses, may be due to the improved safety of a chirally controlled oligonucleotide composition.

In some embodiments, with their improved delivery (and other properties), provided compositions can be administered in lower dosages and/or with lower frequency to achieve biological effects, for example, clinical efficacy.

A single dose can contain various amounts of oligonucleotides. In some embodiments, a single dose can contain various amounts of a type of chirally controlled oligonucleotide, as desired suitable by the application. In some embodiments, a single dose contains about 1, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300 or more (e.g., about 350, 400, 450, 500, 550, 600, 650, 700, 750, 800, 850, 900, 950, 1000 or more) mg of a type of chirally controlled oligonucleotide. In some embodiments, a single dose contains about 1 mg of a type of chirally controlled oligonucleotide. In some embodiments, a single dose contains about 5 mg of a type of chirally controlled oligonucleotide. In some embodiments, a single dose contains about 10 mg of a type of chirally controlled oligonucleotide. In some embodiments, a single dose contains about 15 mg of a type of chirally controlled oligonucleotide. In some embodiments, a single dose contains about 20 mg of a type of chirally controlled oligonucleotide. In some embodiments, a single dose contains about 50 mg of a type of chirally controlled oligonucleotide. In some embodiments, a single dose contains about 100 mg of a type of chirally controlled oligonucleotide. In some embodiments, a single dose contains about 150 mg of a type of chirally controlled oligonucleotide. In some embodiments, a single dose contains about 200 mg of a type of chirally controlled oligonucleotide. In some embodiments, a single dose contains about 250 mg of a type of chirally controlled oligonucleotide. In some embodiments, a single dose contains about 300 mg of a type of chirally controlled oligonucleotide. In some embodiments, a chirally controlled oligonucleotide is administered at a lower amount in a single dose, and/or in total dose, than a chirally uncontrolled oligonucleotide. In some embodiments, a chirally controlled oligonucleotide is administered at a lower amount in a single dose, and/or in total dose, than a chirally uncontrolled oligonucleotide due to improved efficacy. In some embodiments, a chirally controlled oligonucleotide is administered at a higher amount in a single dose, and/or in total dose, than a chirally uncontrolled oligonucleotide. In some embodiments, a chirally controlled oligonucleotide is administered at a higher amount in a single dose, and/or in total dose, than a chirally uncontrolled oligonucleotide due to improved safety.

### Biologically Active Oligonucleotides

In some embodiments, the present disclosure encompasses oligonucleotides which capable of acting as single-stranded RNAi agents.

In some embodiments, provided compositions include one or more oligonucleotides fully or partially complementary to strand of: structural genes, genes control and/or termination regions, and/or self-replicating systems such as viral or plasmid DNA. In some embodiments, provided compositions include one or more oligonucleotides that are or act as RNAi agents or other RNA interference reagents (RNAi agents or iRNA agents), shRNA, antisense oligonucleotides, self-cleaving RNAs, ribozymes, fragment thereof and/or variants thereof (such as Peptidyl transferase 23S rRNA, RNase P, Group I and Group II introns, GIR1 branching ribozymes, Leadzyme, Hairpin ribozymes, Hammerhead ribozymes, HDV ribozymes, Mammalian CPEB3 ribozyme, VS ribozymes, glmS ribozymes, CoTC ribozyme, etc.), microRNAs, microRNA mimics, supermirs, aptamers, antimirs, antagomirs, Ul adaptors, triplex-forming oligonucleotides, RNA activators, long non-coding RNAs, short non-coding RNAs (e.g., piRNAs), immunomodulatory oligonucleotides (such as immunostimulatory oligonucleotides, immunoinhibitory oligonucleotides), GNA, LNA, ENA, PNA, TNA, morpholinos, G-quadruplex (RNA and DNA), antiviral oligonucleotides, and decoy oligonucleotides.

In some embodiments, provided compositions include one or more hybrid (e.g., chimeric) oligonucleotides. In the context of the present disclosure, the term “hybrid” broadly refers to mixed structural elements of oligonucleotides. Hybrid oligonucleotides may refer to, for example, (1) an APOC3 oligonucleotide molecule having mixed classes of nucleotides, e.g., part DNA and part RNA within the single molecule (e.g., DNA-RNA); (2) complementary pairs of nucleic acids of different classes, such that DNA:RNA base pairing occurs either intramolecularly or intermolecularly; or both; (3) an APOC3 oligonucleotide with two or more kinds of the backbone or internucleotide linkages.

In some embodiments, provided compositions include one or more oligonucleotide that comprises more than one classes of nucleic acid residues within a single molecule. For example, in any of the embodiments described herein, an APOC3 oligonucleotide may comprise a DNA portion and an RNA portion. In some embodiments, an APOC3 oligonucleotide may comprise a unmodified portion and modified portion.

Provided oligonucleotide compositions can include oligonucleotides containing any of a variety of modifications, for example as described herein. In some embodiments, particular modifications are selected, for example, in light of intended use. In some embodiments, it is desirable to modify one or both strands of a double-stranded oligonucleotide (or a double-stranded portion of a single-stranded oligonucleotide). In some embodiments, the two strands (or portions) include different modifications. In some embodiments, the two strands include the same modifications. One of skill in the art will appreciate that the degree and type of modifications enabled by methods of the present disclosure allow for numerous permutations of modifications to be made. Examples of such modifications are described herein and are not meant to be limiting.

The phrase “antisense strand” as used herein, refers to an APOC3 oligonucleotide that is substantially or 100% complementary to a target sequence of interest. The phrase “antisense strand” includes the antisense region of both oligonucleotides that are formed from two separate strands, as well as unimolecular oligonucleotides that are capable of forming hairpin or dumbbell type structures. In reference to a double-stranded RNAi agent such as a siRNA, the antisense strand is the strand preferentially incorporated into RISC, and which targets RISC-mediated knockdown of a RNA target. In reference to a double-stranded RNAi agent, the terms “antisense strand” and “guide strand” are used interchangeably herein; and the terms “sense strand” or “passenger strand” are used interchangeably herein in reference to the strand which is not the antisense strand.

The phrase “sense strand” refers to an APOC3 oligonucleotide that has the same nucleoside sequence, in whole or in part, as a target sequence such as a messenger RNA or a sequence of DNA.

By “target sequence” is meant any nucleic acid sequence whose expression or activity is to be modulated. The target nucleic acid can be DNA or RNA, such as endogenous DNA or RNA, viral DNA or viral RNA, or other RNA encoded by a gene, virus, bacteria, fungus, mammal, or plant. In some embodiments, a target sequence is associated with a disease or disorder. In reference to RNA interference and RNase H-mediated knockdown, a target sequence is generally a RNA target sequence.

By “specifically hybridizable” and “complementary” is meant that a nucleic acid can form hydrogen bond(s) with another nucleic acid sequence by either traditional Watson-Crick or other non-traditional types. In reference to the nucleic molecules of the present disclosure, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LIT pp. 123-133; Frier et al., 1986, Proc. Nat. Acad. Sci. USA 83: 9373-9377; Turner et al., 1987, J. Am. Chem. Soc. 109:3783-3785)

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, e.g., under physiological conditions in the case of in vivo assays or therapeutic treatment, or in the case of in vitro assays, under conditions in which the assays are performed. In some embodiments, non-target sequences differ from corresponding target sequences by at least 5 nucleotides.

When used as therapeutics, a provided oligonucleotide is administered as a pharmaceutical composition. In some embodiments, the pharmaceutical composition comprises a therapeutically effective amount of a provided oligonucleotide comprising, or a pharmaceutically acceptable salt thereof, and at least one pharmaceutically acceptable inactive ingredient selected from pharmaceutically acceptable diluents, pharmaceutically acceptable excipients, and pharmaceutically acceptable carriers. In some embodiments, the pharmaceutical composition is formulated for intravenous injection, oral administration, buccal administration, inhalation, nasal administration, topical administration, ophthalmic administration or otic administration. In further embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

### Pharmaceutical Compositions

When used as therapeutics, a provided oligonucleotide or oligonucleotide composition described herein is administered as a pharmaceutical composition. In some embodiments, the pharmaceutical composition comprises a therapeutically effective amount of a provided oligonucleotides, or a pharmaceutically acceptable salt thereof, and at least one pharmaceutically acceptable inactive ingredient selected from pharmaceutically acceptable diluents, pharmaceutically acceptable excipients, and pharmaceutically acceptable carriers. In some embodiments, the pharmaceutical composition is formulated for intravenous injection, oral administration, buccal administration, inhalation, nasal administration, topical administration, ophthalmic administration or otic administration. In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

In some embodiments, the present disclosure provides a pharmaceutical composition comprising chirally controlled oligonucleotide, or composition thereof, in admixture with a pharmaceutically acceptable excipient. One of skill in the art will recognize that the pharmaceutical compositions include the pharmaceutically acceptable salts of the chirally controlled oligonucleotide, or composition thereof, described above.

A variety of supramolecular nanocarriers can be used to deliver nucleic acids. Example nanocarriers include, but are not limited to liposomes, cationic polymer complexes and various polymeric. Complexation of nucleic acids with various polycations is another approach for intracellular delivery; this includes use of PEGlyated polycations, polyethyleneamine (PEI) complexes, cationic block co-polymers, and dendrimers. Several cationic nanocarriers, including PEI and polyamidoamine dendrimers help to release contents from endosomes. Other approaches include use of polymeric nanoparticles, microspheres, liposomes, dendrimers, biodegradable polymers, conjugates, prodrugs, inorganic colloids such as sulfur or iron, antibodies, implants, biodegradable implants, biodegradable microspheres, osmotically controlled implants, lipid nanoparticles, emulsions, oily solutions, aqueous solutions, biodegradable polymers, poly(lactide-coglycolic acid), poly(lactic acid), liquid depot, polymer micelles, quantum dots and lipoplexes. In some embodiments, an APOC3 oligonucleotide is conjugated to another molecular.

Additional nucleic acid delivery strategies are known in addition to the example delivery strategies described herein.

In therapeutic and/or diagnostic applications, the compounds of the disclosure can be formulated for a variety of modes of administration, including systemic and topical or localized administration. Techniques and formulations generally may be found in Remington, The Science and Practice of Pharmacy, (20th ed. 2000).

Provided oligonucleotides, and compositions thereof, are effective over a wide dosage range. For example, in the treatment of adult humans, dosages from about 0.01 to about 1000 mg, from about 0.5 to about 100 mg, from about 1 to about 50 mg per day, and from about 5 to about 100 mg per day are examples of dosages that may be used. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

In some embodiments, a provided single-stranded RNAi agent is formulated in a pharmaceutical composition described in U.S. Applications No. 61/774,759; 61/918,175, filed Dec. 19, 2013; 61/918,927; 61/918,182; 61/918,941; 62/025,224; 62/046,487; or International Applications No. PCT/US04/042911; PCT/EP2010/070412; or PCT/I B2014/059503.

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection.

The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure may also be formulated by methods known to those of skill in the art, and may include, for example, but not limited to, examples of solubilizing, diluting, or dispersing substances such as, saline, preservatives, such as benzyl alcohol, absorption promoters, and fluorocarbons.

In certain embodiments, oligonucleotides and compositions are delivered to the CNS. In certain embodiments, oligonucleotides and compositions are delivered to the cerebrospinal fluid. In certain embodiments, oligonucleotides and compositions are administered to the brain parenchyma. In certain embodiments, oligonucleotides and compositions are delivered to an animal/subject by intrathecal administration, or intracerebroventricular administration. Broad distribution of oligonucleotides and compositions, described herein, within the central nervous system may be achieved with intraparenchymal administration, intrathecal administration, or intracerebroventricular administration.

In certain embodiments, parenteral administration is by injection, by, e.g., a syringe, a pump, etc. In certain embodiments, the injection is a bolus injection. In certain embodiments, the injection is administered directly to a tissue, such as striatum, caudate, cortex, hippocampus and cerebellum.

In certain embodiments, methods of specifically localizing a pharmaceutical agent, such as by bolus injection, decreases median effective concentration (EC50) by a factor of 20, 25, 30, 35, 40, 45 or 50. In certain embodiments, the pharmaceutical agent in an antisense compound as further described herein. In certain embodiments, the targeted tissue is brain tissue. In certain embodiments the targeted tissue is striatal tissue. In certain embodiments, decreasing EC50 is desirable because it reduces the dose required to achieve a pharmacological result in a patient in need thereof.

In certain embodiments, an antisense oligonucleotide is delivered by injection or infusion once every month, every two months, every 90 days, every 3 months, every 6 months, twice a year or once a year.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of an active compound into preparations which can be used pharmaceutically. The preparations formulated for oral administration may be in the form of tablets, dragees, capsules, or solutions.

Pharmaceutical preparations for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, an active compound may be dissolved or suspended in suitable liquids, such as fatty oils, liquid paraffin, or liquid polyethylene glycols (PEGs). In addition, stabilizers may be added.

A composition can be obtained by combining an active compound with a lipid. In some embodiments, the lipid is conjugated to an active compound. In some embodiments, the lipid is not conjugated to an active compound. In some embodiments, a lipid comprises a C₁₀-C₄₀ linear, saturated or partially unsaturated, aliphatic chain. In some embodiments, a lipid comprises a C₁₀-C₄₀ linear, saturated or partially unsaturated, aliphatic chain, optionally substituted with one or more C₁₋₄ aliphatic group. In some embodiments, the lipid is selected from the group consisting of: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl. In some embodiments, a lipid has a structure of any of:

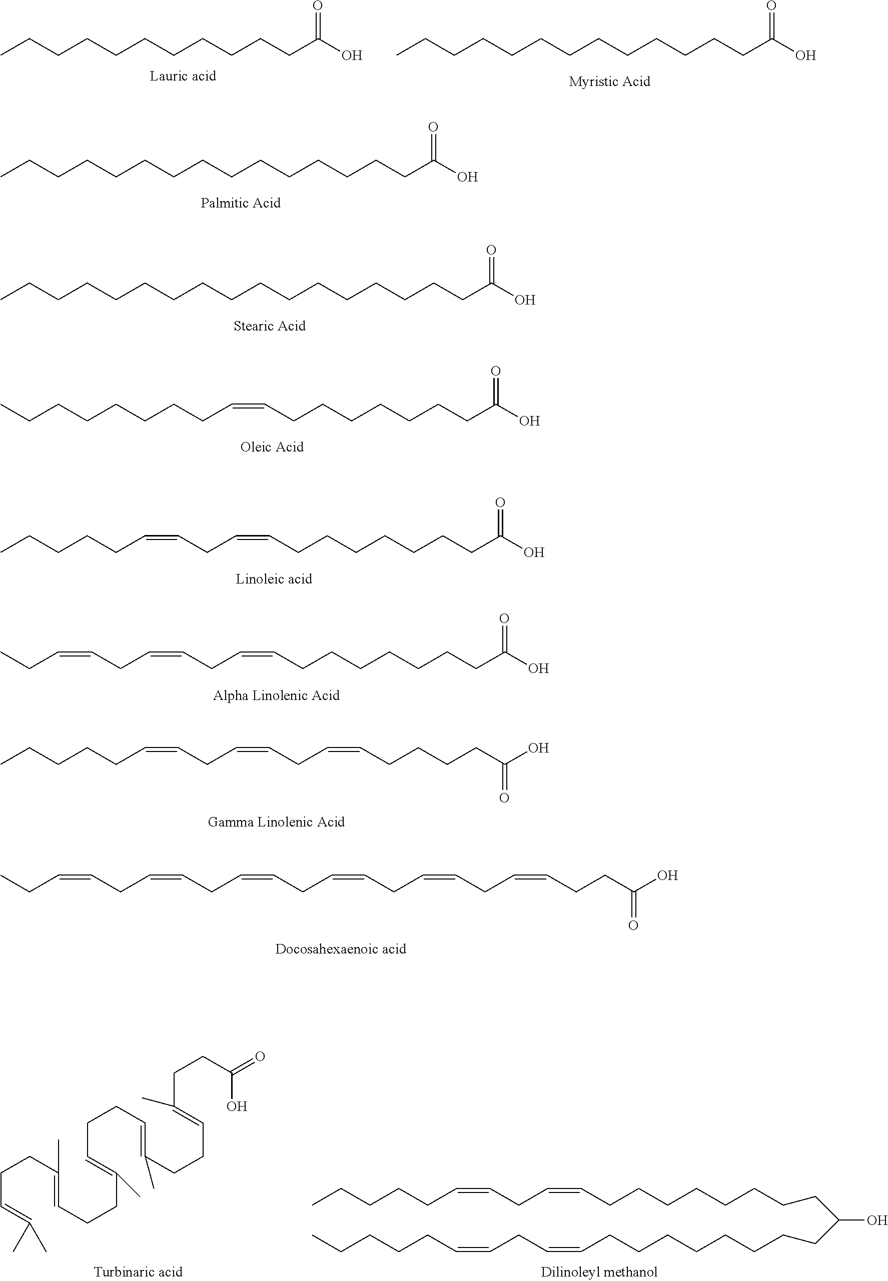

In some embodiments, an active compound is any oligonucleotide or other nucleic acid described herein. In some embodiments, an active compound is a nucleic acid of a sequence comprising or consisting of any sequence of any nucleic acid listed in Table 1A. In some embodiments, a composition comprises a lipid and an an active compound, and further comprises another component selected from: another lipid, and a targeting compound or moiety. In some embodiments, a lipid includes, without limitation: an amino lipid; an amphipathic lipid; an anionic lipid; an apolipoprotein; a cationic lipid; a low molecular weight cationic lipid; a cationic lipid such as CLinDMA and DLinDMA; an ionizable cationic lipid; a cloaking component; a helper lipid; a lipopeptide; a neutral lipid; a neutral zwitterionic lipid; a hydrophobic small molecule; a hydrophobic vitamin; a PEG-lipid; an uncharged lipid modified with one or more hydrophilic polymers; phospholipid; a phospholipid such as 1,2-dioleoyl-sn-glycero-3-phosphoethanolamine; a stealth lipid; a sterol; a cholesterol; and a targeting lipid; and any other lipid described herein or reported in the art. In some embodiments, a composition comprises a lipid and a portion of another lipid capable of mediating at least one function of another lipid. In some embodiments, a targeting compound or moiety is capable of targeting a compound (e.g., a composition comprising a lipid and a active compound) to a particular cell or tissue or subset of cells or tissues. In some embodiments, a targeting moiety is designed to take advantage of cell- or tissue-specific expression of particular targets, receptors, proteins, or other subcellular components; In some embodiments, a targeting moiety is a ligand (e.g., a small molecule, antibody, peptide, protein, carbohydrate, aptamer, etc.) that targets a composition to a cell or tissue, and/or binds to a target, receptor, protein, or other subcellular component.

Certain example lipids for use in preparation of a composition for delivery of an active compound allow (e.g., do not prevent or interfere with) the function of an active compound. Non-limiting example lipids include: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl.

As described in the present disclosure, lipid conjugation, such as conjugation with fatty acids, may improve one or more properties of oligonucleotides.

In some embodiments, a composition for delivery of an active compound is capable of targeting an active compound to particular cells or tissues, as desired. In some embodiments, a composition for delivery of an active compound is capable of targeting an active compound to a muscle cell or tissue. In some embodiments, the present disclosure pertains to compositions and methods related to delivery of active compounds, wherein the compositions comprise an active compound a lipid. In various embodiments to a muscle cell or tissue, the lipid is selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl. The example lipids used include stearic acid, oleic acid, alpha-linolenic acid, gamma-linolenic acids, cis-DHA, turbinaric acid and dilinoleyl acid. In these Tables, “TBD” indicates that the particular composition was effective for delivery, but the numerical results were outside the standard range, and thus the final results remain to be determined; however, the compositions indicated as “TBD” in the Tables were efficacious at delivery of an active compound.

A composition comprising an active compound and any of: stearic acid, oleic acid, alpha-linolenic acid, gamma-linolenic acid, cis-DHA or turbinaric acid, was able to deliver an active compound to gastrocnemius muscle tissue. A composition comprising an active compound and any of: stearic acid, alpha-linolenic, gamma-linolenic, cis-DHA, or turbinaric acid, was able to deliver an active compound to heart muscle tissue. A composition comprising an active compound and any of: stearic acid, oleic acid, alpha-linolenic acid, gamma-linolenic acid, cis-DHA or turbinaric acid, was able to deliver an active compound to quadriceps muscle tissue. A composition comprising an active compound and any of: stearic, oleic, alpha-linolenic, gamma-linolenic, cis-DHA, or turbinaric acid was able to deliver an active compound to the gastrocnemius muscle tissue. A composition comprising an active compound and any of: stearic acid, alpha-linolenic, gamma-linolenic, cis-DHA, or turbinaric acid was able to deliver an active compound to heart muscle tissue. A composition comprising an active compound and any of: dilinoleyl, stearic acid, oleic acid, alpha-linolenic, gamma-linolenic, cis-DHA or turbinaric acid was able to delivery an active compound to the diaphragm muscle tissue.

Thus: A composition comprising a lipid, selected from: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, and an active compound is capable of delivering an active compound to extra-hepatic cells and tissues, e.g., muscle cells and tissues.

Depending upon the particular condition, or disease state, to be treated or prevented, additional therapeutic agents, which are normally administered to treat or prevent that condition, may be administered together with oligonucleotides of this disclosure. For example, chemotherapeutic agents or other anti-proliferative agents may be combined with the oligonucleotides of this disclosure to treat proliferative diseases and cancer. Examples of known chemotherapeutic agents include, but are not limited to, adriamycin, dexamethasone, vincristine, cyclophosphamide, fluorouracil, topotecan, taxol, interferons, and platinum derivatives.

### Example Uses

In some embodiments, the present disclosure encompasses the use of a composition comprising a lipid and an APOC3 oligonucleotide or a single-stranded RNAi agent. In some embodiments, the present disclosure provides methods for delivering an APOC3 oligonucleotide or a single-stranded RNAi agent to a target location comprising administering a provided composition. In some embodiments, a provided method delivers an APOC3 oligonucleotide or a single-stranded RNAi agent into a cell. In some embodiments, a provided method delivers an APOC3 oligonucleotide or a single-stranded RNAi agent into a muscle cell. In some embodiments, a provided method delivers an APOC3 oligonucleotide or a single-stranded RNAi agent into a cell within a tissue. In some embodiments, a provided method delivers an APOC3 oligonucleotide or a single-stranded RNAi agent into a cell within an organ. In some embodiments, a provided method delivers an APOC3 oligonucleotide or a single-stranded RNAi agent into a cell within a subject, comprising administering to the subject a provided composition. In some embodiments, a provided method delivers an APOC3 oligonucleotide or a single-stranded RNAi agent into cytoplasm. In some embodiments, a provided method delivers an APOC3 oligonucleotide or a single-stranded RNAi agent into nucleus.

In some embodiments, the present disclosure pertains to methods related to the delivery of an APOC3 oligonucleotide or a single-stranded RNAi agent to a cell or tissue, or a cell or tissue in a mammal (e.g., a human subject), which method pertains to a use of a composition comprising a biological agent and a lipid. any one or more additional components selected from: a polynucleotide, a dye, an intercalating agent (e.g. an acridine), a cross-linker (e.g. psoralene, or mitomycin C), a porphyrin (e.g., TPPC4, texaphyrin, or Sapphyrin), a polycyclic aromatic hydrocarbon (e.g., phenazine, or dihydrophenazine), an artificial endonuclease, a chelating agent, EDTA, an alkylating agent, a phosphate, an amino, a mercapto, a PEG (e.g., PEG-40K), MPEG, [MPEG]₂, a polyamino, an alkyl, a substituted alkyl, a radiolabeled marker, an enzyme, a hapten (e.g. biotin), a transport/absorption facilitator (e.g., aspirin, vitamin E, or folic acid), a synthetic ribonuclease, a protein, e.g., a glycoprotein, or peptide, e.g., a molecule having a specific affinity for a co-ligand, or antibody e.g., an antibody, a hormone, a hormone receptor, a non-peptidic species, a lipid, a lectin, a carbohydrate, a vitamin, a cofactor, or a drug. In some embodiments, the present disclosure pertains to compositions or methods related to a composition comprising an APOC3 oligonucleotide or a single-stranded RNAi agent and a lipid comprising a C₁₀-C₆₀ linear, saturated or partially unsaturated, aliphatic chain. In some embodiments, the present disclosure pertains to compositions or methods related to a composition comprising an APOC3 oligonucleotide or a single-stranded RNAi agent and a lipid comprising a C₁₀-C₆₀ linear, saturated or partially unsaturated, aliphatic chain, optionally substituted with one or more C₁₋₄ aliphatic group. In some embodiments, the present disclosure provides chirally controlled oligonucleotide compositions and a lipid selected from the group consisting of: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl, wherein the composition is suitable for delivery of the oligonucleotide to a muscle cell or tissue, or a muscle cell or tissue in a mammal (e.g., a human subject). In some embodiments, an APOC3 oligonucleotide or a single-stranded RNAi agent is an APOC3 oligonucleotide comprising one or more chiral internucleotidic linkages, and a provided composition is an APOC3 oligonucleotide or a single-stranded RNAi agent. In some embodiments, an APOC3 oligonucleotide or a single-stranded RNAi agent is an APOC3 oligonucleotide comprising one or more chiral internucleotidic linkages, and a provided composition is a non-chirally controlled oligonucleotide composition of the oligonucleotide.

In some embodiments, the present disclosure pertains to a method of delivering an APOC3 oligonucleotide or a single-stranded RNAi agent to a cell or tissue, wherein the method comprises steps of: providing a composition comprising an APOC3 oligonucleotide or a single-stranded RNAi agent and a lipid; and contacting the cell or tissue with the composition; in some embodiments, the present disclosure pertains to a method of administering an APOC3 oligonucleotide or a single-stranded RNAi agent to a subject, wherein the method comprises steps of: providing a composition comprising an APOC3 oligonucleotide or a single-stranded RNAi agent and a lipid; and administering the composition to the subject. In some embodiments, a lipid comprises a C₁₀-C₆₀ linear, saturated or partially unsaturated, aliphatic chain. In some embodiments, a lipid comprises a C₁₀-C₆₀ linear, saturated or partially unsaturated, aliphatic chain, optionally substituted with one or more C₁₋₄ aliphatic group. In some embodiments, the lipid is selected from the group consisting of: lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl.

In some embodiments, an APOC3 oligonucleotide or a single-stranded RNAi agent is an APOC3 oligonucleotide, whose sequence is or comprises an element that is substantially complementary to a targeted element in a cellular nucleic acid. In some embodiments, a targeted element is or comprises a sequence element that is associated with a muscle disease, disorder or condition. In some embodiments, a muscle disease, disorder or condition is DMD. In some embodiments, a cellular nucleic acid is or comprises a transcript. In some embodiments, a cellular nucleic acid is or comprises a primary transcript. In some embodiments, a cellular nucleic acid is or comprises a genomic nucleic acid. The present disclosure encompasses the recognition that certain lipids and other compounds are useful for delivery of single-stranded RNAi agents to cells and tissues, e.g., in a mammal or human subject. Many technologies for delivering such agents can suffer from an inability to target desired cells or tissues.

Delivery of single-stranded RNAi agents to tissues outside the liver remains difficult. Juliano reported that, despite advances at the clinical level, effective delivery of oligonucleotides in vivo remains a major challenge, especially at extra-hepatic sites. Juliano 2016 Nucl. Acids Res. Doi: 10.1093/nar/gkw236. Lou also reported that delivery of RNAi agent to organs beyond the liver remains the biggest hurdle to using the technology for a host of diseases. Lou 2014 SciBX 7(48); doi:10.1038/scibx.2014.1394.

The present disclosure encompasses certain surprising findings, including that certain lipids and other compounds are particularly effective at delivering single-stranded RNAi agents, including oligonucleotides, to particular cells and tissues, including cells and tissues outside the liver, including, as non-limiting examples, muscle cells and tissues.

In some embodiments, provided compositions alter single-stranded RNA interference system so that an undesired target and/or biological function are suppressed. In some embodiments, in such cases provided composition can also induce cleavage of the transcript after hybridization.

In some embodiments, provided compositions alter single-stranded RNA interference system so a desired target and/or biological function is enhanced. In some embodiments, provided compositions, by incorporating chemical modifications, stereochemistry and/or combinations thereof, effectively suppress or prevent cleavage of a target transcript after contact.

In some embodiments, each oligonucleotide of a plurality comprises one or more modified sugar moieties and modified internucleotidic linkages. In some embodiments, each oligonucleotide of a plurality comprises two or more modified sugar moieties. In some embodiments, each oligonucleotide of a plurality comprises three or more modified sugar moieties. In some embodiments, each oligonucleotide of a plurality comprises four or more modified sugar moieties. In some embodiments, each oligonucleotide of a plurality comprises five or more modified sugar moieties. In some embodiments, each oligonucleotide of a plurality comprises ten or more modified sugar moieties. In some embodiments, each oligonucleotide of a plurality comprises about 15 or more modified sugar moieties. In some embodiments, each oligonucleotide of a plurality comprises about 20 or more modified sugar moieties. In some embodiments, each oligonucleotide of a plurality comprises about 25 or more modified sugar moieties.

### EXEMPLIFICATION

The foregoing has been a description of certain non-limiting embodiments of the disclosure. Accordingly, it is to be understood that the embodiments of the disclosure herein described are merely illustrative of the application of the principles of the disclosure. Reference herein to details of the illustrated embodiments is not intended to limit the scope of the claims.

Certain methods for preparing oligonucleotides and oligonucleotide compositions are widely known in the art and can be utilized in accordance with the present disclosure, including but not limited to those described in WO/2010/064146, WO/2011/005761, WO/2013/012758, WO/2014/010250, US2013/0178612, WO/2014/012081, WO/2015/107425, WO/2017/015555, and WO/2017/062862, the methods and reagents of each of which are incorporated herein by reference.

Applicant describes herein certain examples of provided oligonucleotide and compositions thereof, and methods for preparing, assessing, assaying, and using, etc., certain provided oligonucleotides and compositions thereof.

### Example 1. Example Protocols for Assessing Oligonucleotides

As a personal having ordinary skill in the art appreciates, many technologies (e.g., reagents, methods, etc.) can be utilized to assess activities and properties of provided oligonucleotides. Below is one example protocol describing reverse transfection of oligonucleotides (using certain oligonucleotides that can function as ssRNAi as examples) in 96 Well Plate format using Lipofectamine® 2000 (Invitrogen) for assessing oligonucleotide activities in cells:
1. Prepare each ssRNAi, preferably in multiple (e.g., 8) doses, e.g., in a final volume of 25 uL. Example initial concentration could be 150 nM; serial dilution, for example in Opti-MEM® medium without serum, typically by a factor of 4.
2. Lipofectamine® 2000 is desirably mixed gently before use, then diluted 0.25 μl Lipofectamine® 2000 in 25 μl Opti-MEM® medium without serum in a separate vessel. Further gentle mixing can be followed by incubation, e.g., for 5 minutes at room temperature.
3. After incubation, diluted Lipofectamine® 2000 (e.g., 25 uL) can be added to the (diluted) ssRNAi molecules (typically comparable volume, e.g., 25 uL). The combination is desirably mixed gently and may be incubated, e.g., for 15 minutes at room temperature, to allow complex formation to occur.
4. Complexes are then contacted with cells, for example by adding 100 μl complete growth medium without antibiotics with 15,000 Hep3B cells to each ssRNAi molecule-Lipofectamine® 2000 complex. This gives a final volume of 150 μl, and final oligo concentrations are 25, 6.25, 1.56, 0.39, 0.097, 0.024, 0.0061, and 0.0015 nM. Mix gently by rocking the plate back and forth.
5. Cells are incubated, e.g., at 37° C. in a CO₂ incubator for 48 hours.
6. Cells are harvested and mRNA is isolated, e.g., using TurboCapture mRNA kit (Qiagen), as per vendor provided protocol.
7. CDNA is prepared, e.g., using Roche CDNA synthesis Kit (Roche), as per vendor provided protocol.
8. Target knockdown is quantified, e.g., by Taqman assays using gene-specific Taqman probes multiplexed with HPRT1 probes, in LightCycler® 480 Probes Master mix (Roche), as per vendor provided protocol. Typically, data are normalized, for example relative to a housekeeping gene such as HPRT1 (Hypoxanthine Phosphoribosyltransferase 1).
9. If multiple dose strengths/concentrations were utilized, dose-response curves can be prepared for each ssRNAi agent, e.g., using Prism Software. IC₅₀ can be determined if desired.

Similar protocols can be used for different oligonucleotides targeting other genes and can use different cells.

Alternatively or additionally, one or more activities and properties of oligonucleotides can be assessed using other technologies (e.g., reagents, kits, methods, etc.) in accordance with the present disclosure. Certain data generated from various types of assays are provided in the Tables, demonstrating, for example, unexpectedly high activities, stability, selectivity, etc., of presently provided technologies.

Various models are available for assessing provided technologies in subjects. In some embodiments, provided technologies show high activities, stability, and/or selectivity when administered to animals. Those skilled in the art are aware of animal systems that are considered to be relevant to and/or predictive for certain relevant human diseases, disorders and/or conditions that might benefit from oligonucleotide therapy as described herein.

### Example 2. Example IC50 of Certain Provided Oligonucleotides

IC50 of certain oligonucleotides (which may function as single-stranded RNAi agents to APOC3) measured using a protocol such as that presented in Example 1 are provided in the following Table.

| Oligonucleotide | IC50 (nM) |
|---|---|
| WV-5291 | 0.372 |
| WV-6411 | 0.121 |
| WV-6412 | 0.129 |
| WV-6413 | 0.089 |
| WV-6414 | 0.144 |
| WV-6415 | 0.169 |
| WV-6416 | 0.206 |
| WV-6417 | 0.158 |
| WV-6418 | 0.164 |
| WV-6419 | 0.121 |
| WV-6420 | 0.230 |
| WV-6421 | 0.216 |
| WV-6422 | 0.301 |
| WV-6423 | 0.616 |
| WV-6424 | 0.226 |
| WV-6425 | 0.412 |
| WV-6426 | 0.325 |
| WV-6427 | 0.170 |
| WV-6428 | 0.249 |
| WV-6429 | 0.237 |
| WV-6430 | 0.204 |
| WV-6764 | 0.609 |
| WV-6765 | 0.709 |

### Example 3. Example Compounds for Incorporating Moieties—Synthesis of Tri-Antennary GalNAc (with C12, C5, or Triazine Linkers)

In some embodiments, the present disclosure provides technologies (e.g., reagents, methods, conjugates, etc.) for incorporating various moieties (e.g., carbohydrate moieties, lipid moieties, targeting moieties, etc.) into provided oligonucleotide. Described herein are certain examples for incorporating carbohydrate moieties. In some embodiments, a carbohydrate moiety may function as a targeting moiety.

### Example 3-1. Synthesis of 1-(((2R,3R,4R,5R,6R)-3-acetamido-4,5-diacetoxy-6-(acetoxymethyl)tetrahydro-2H-pyran-2-yl)oxy)-16,16-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic acid

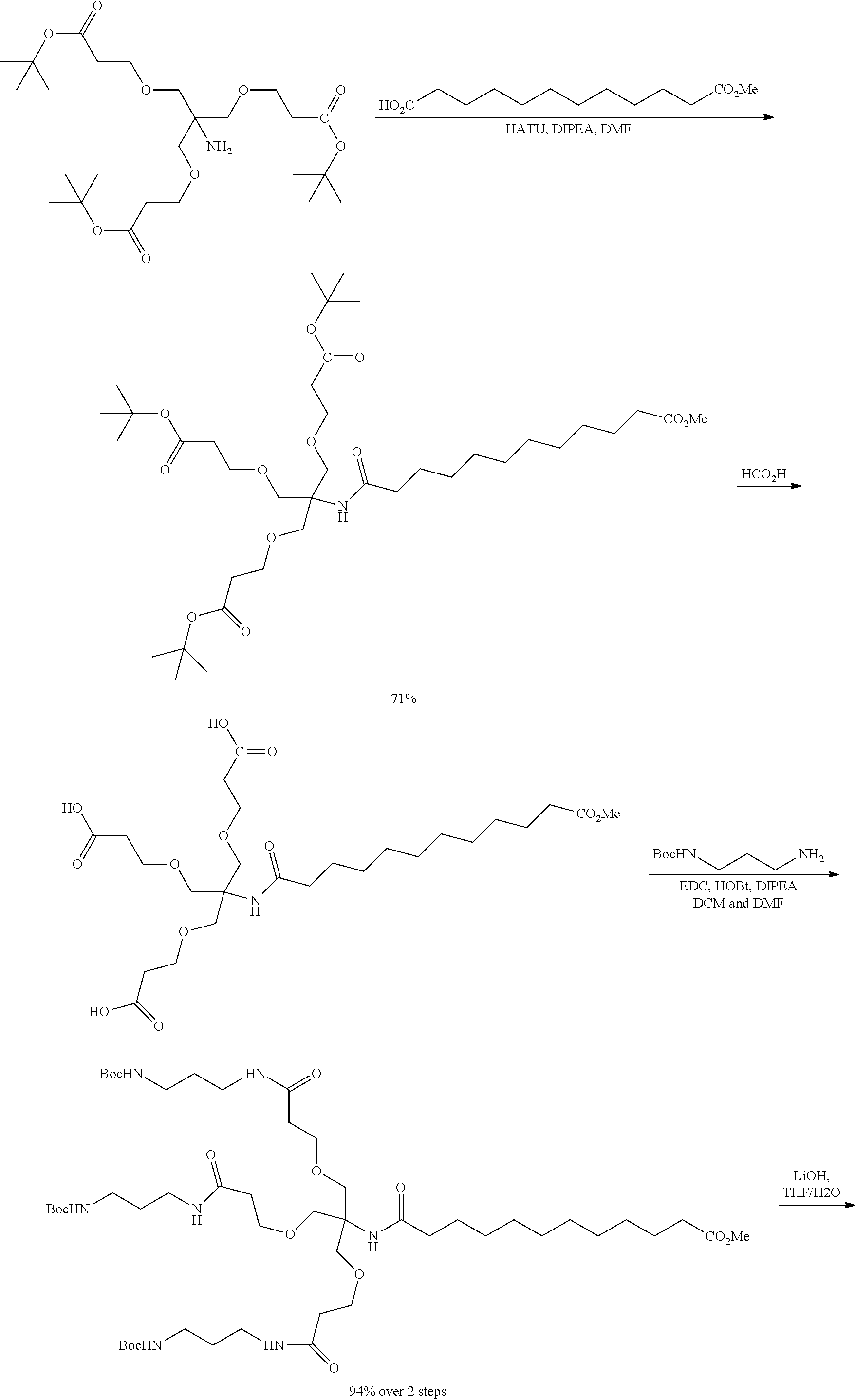

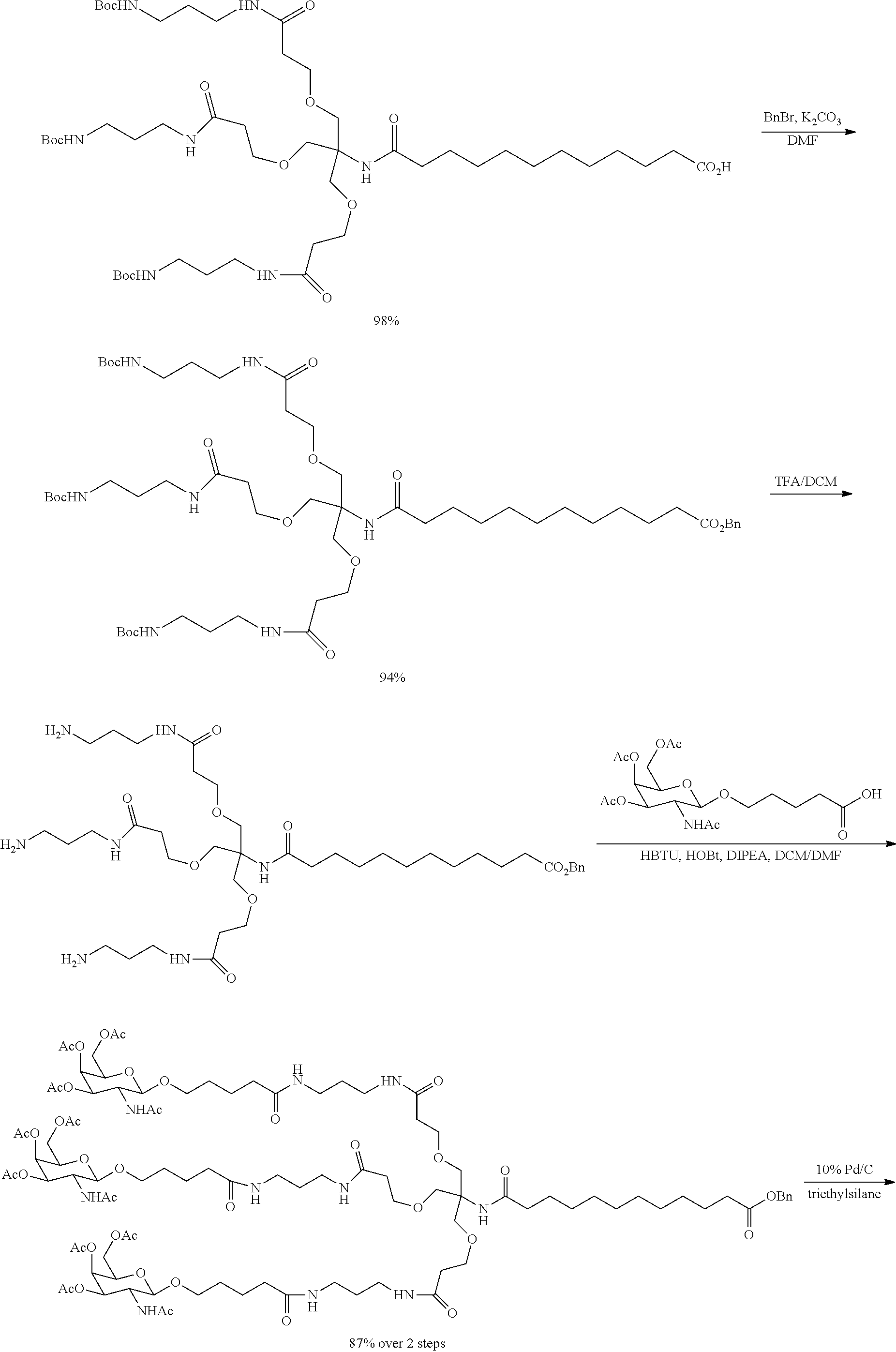

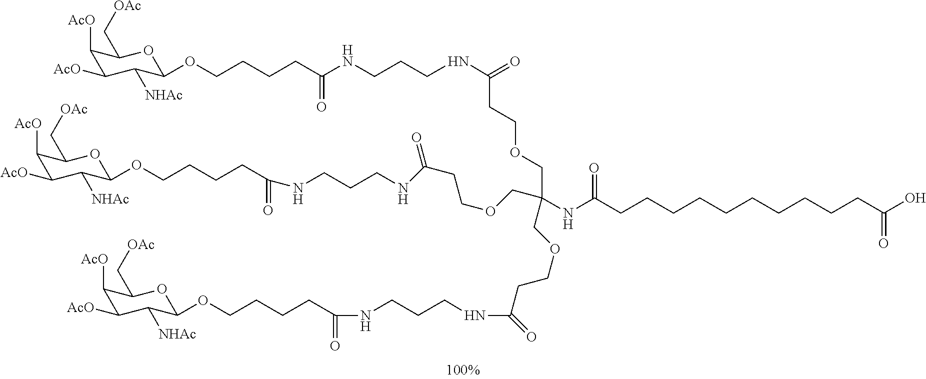 [SMILES: CC(=O)NC1C(OCCCCC(=O)NCCCNC(=O)CCOCC(COCCC(=O)NCCCNC(=O)CCCCOC2OC(COC(C)=O)C(OC(C)=O)C(OC(C)=O)C2NC(C)=O)(COCCC(=O)NCCCNC(=O)CCCCOC2OC(COC(C)=O)C(OC(C)=O)C(OC(C)=O)C2NC(C)=O)NC(=O)CCCCCCCCCCC(=O)O)OC(COC(C)=O)C(OC(C)=O)C1OC(C)=O]

### Step 1

To a solution of di-tert-butyl 3,3′-((2-amino-2-((3-(tert-butoxy)-3-oxopropoxy)methyl)propane-1,3-diyl)bis(oxy))dipropanoate (5.0 g, 9.89 mmol) and 12-methoxy-12-oxododecanoic acid (2.416 g, 9.89 mmol) in DMF (45 mL) was added HATU (3.76 g, 9.89 mmol) and DIPEA (2.58 ml, 14.83 mmol). The reaction mixture was stirred at room temperature for 5 hrs. Solvent was concentrated under reduced pressure, and diluted with brine, extracted with EtOAc, dried over anhydrous sodium sulfate, and concentrated to give a residue, which was purified by ISCO (120 g gold silica gel cartridge) eluting with 10% EtOAc in hexane to 40% EtOAc in hexane to give di-tert-butyl 3,3′-((2-((3-(tert-butoxy)-3-oxopropoxy)methyl)-2-(12-methoxy-12-oxododecanamido)propane-1,3-diyl)bis(oxy))dipropanoate (5.13 g, 7.01 mmol, 70.9% yield) as a colorless oil. ¹H NMR (400 MHz, Chloroform-d) δ 6.03 (s, 1H), 3.74-3.61 (m, 15H), 2.45 (t, J=6.3 Hz, 6H), 2.31 (td, J=7.5, 3.9 Hz, 2H), 2.19-2.10 (m, 2H), 1.64-1.59 (m, 4H), 1.46 (s, 27H), 1.32-1.24 (m, 12H); MS (ESI), 732.6 (M+H)+.

### Step 2

A solution of di-tert-butyl 3,3′-((2-((3-(tert-butoxy)-3-oxopropoxy)methyl)-2-(12-methoxy-12-oxododecanamido)propane-1,3-diyl)bis(oxy))dipropanoate (5.0 g, 6.83 mmol) in formic acid (50 mL) was stirred at room temperature for 48 hrs. Solvent was evaporated under reduced pressure, co-evaporated with toluene (3×) to give a white solid, which was dried under high vacuum for 2 days. LC-MS and H NMR showed the reaction is not complete. The crude product was redissolved in formic acid (50 mL). The reaction mixture was stirred at room temperature for 24 hrs. LC-MS showed the reaction was complete. Solvent was evaporated under reduced pressure, co-evaporated with toluene (3×), dried over high vacuum to give 3,3′-((2-((2-carboxyethoxy)methyl)-2-(12-methoxy-12-oxododecanamido)propane-1,3-diyl)bis(oxy))dipropanoic acid (4.00 g) as a white solid. MS (ESI): 562.4 (M−H)⁻.

### Step 3

A solution of 3,3′-((2-((2-carboxyethoxy)methyl)-2-(12-methoxy-12-oxododecanamido)propane-1,3-diyl)bis(oxy))dipropanoic acid (3.85 g, 6.83 mmol) and HOBt (3.88 g, 28.7 mmol) in DCM (60 mL) and DMF (15 mL) at 0° C. was added tert-butyl (3-aminopropyl)carbamate (4.76 g, 27.3 mmol), EDAC HCl salt (5.24 g, 27.3 mmol) and DIPEA (8.33 ml, 47.8 mmol). The reaction mixture was stirred at 0° C. for 15 minutes and at room temperature for 20 hrs. LC-MS showed the reaction was not complete. t-Butyl (3-aminopropyl) carbamate (1.59 g, 9.12 mmol) and EDC HCl salt (1.75 g, 9.13 mol) was added into the reaction mixture. The reaction mixture was continually stirred at room temperature for 4 hrs. Solvent was evaporated to give a residue, which was dissolved in EtOAc (300 mL), washed with water (1×), saturated sodium bicarbonate (2×), 10% citric acid (2×) and water, dried over sodium sulfate, and concentrated to give a residue which was purified by ISCO (80 g gold cartridge) eluting with DCM to 30% MeOH in DCM to give methyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oate (6.61 g, 6.40 mmol, 94% yield over 2 steps) as a white solid. MS (ESI): 1033.5 (M+H)+.

### Step 4

To a solution of methyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oate (6.56 g, 6.35 mmol) in THF (75 mL) was added aq. LiOH (0.457 g, 19.06 mmol) in water (25 mL). The mixture was stirred at room temperature for overnight. LC-MS showed the reaction was completed. Solvent was evaporated, acidified using 1 N HCl (45 mL), extracted with DCM (3×), dried over anhydrous sodium sulfate, concentrated to give 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oic acid (6.31 g, 6.20 mmol, 98% yield) as a white solid. MS (ESI): 1019.6 (M+H)⁺.

### Step 5

To a solution of 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oic acid (6.31 g, 6.20 mmol) and (bromomethyl)benzene (1.272 g, 7.44 mmol) in DMF (40 mL) was added K₂CO₃ (2.57 g, 18.59 mmol). The mixture was stirred at 40° C. for 4 hrs and at room temperature for overnight. Solvent was evaporated under reduced pressure. The reaction mixture was diluted with EtOAc, washed with water, dried over anhydrous sodium sulfate, concentrated under reduced pressure to give a residue, which was purified by ISCO (80 g cartridge) eluting with DCM to 30% MeOH in DCM to give benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oate (6.41 g, 5.78 mmol, 93% yield) as a colorless oil. ¹H NMR (400 MHz, DMSO-d₆) δ 7.80 (t, J=5.7 Hz, 3H), 7.39-7.30 (m, 5H), 6.95 (s, 1H), 6.74 (t, J=5.8 Hz, 3H), 5.07 (s, 2H), 3.53 (J, J=7.3 Hz, 6H), 3.51 (s, 6H), 3.02 (q, J=6.7 Hz, 6H), 2.94-2.85 (m, 6H), 2.29 (dt, J=26.1, 6.9 Hz, 8H), 2.02 (q, J=9.7, 8.6 Hz, 2H), 1.56-1.39 (m, 10H), 1.35 (s, 27H), 1.20 (brs, 14H); MS (ESI): 1019.6 (M+H)⁺.

### Step 6

To a solution of benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oate (2.42 g, 2.183 mmol) in DCM (40 mL) was added 2,2,2-trifluoroacetic acid (8 ml, 105 mmol). The reaction mixture was stirred at room temperature for overnight. Solvent was evaporated under reduced pressure, co-evaporated with toluene (2×), triturated with ether, dried under high vacuum for overnight. Directly use TFA salt for next step.

### Step 7

To a solution of 5-(((2R,3R,4R,5R,6R)-3-acetamido-4,5-diacetoxy-6-(acetoxymethyl)tetrahydro-2H-pyran-2-yl)oxy)pentanoic acid (3.91 g, 8.73 mmol), HBTU (3.48 g, 9.17 mmol) and HOBT (1.239 g, 9.17 mmol) in DCM (25 mL) was added DIPEA (6.08 ml, 34.9 mmol) followed by benzyl 12-((1,19-diamino-10-((3-((3-aminopropyl)amino)-3-oxopropoxy)methyl)-5,15-dioxo-8,12-dioxa-4,16-diazanonadecan-10-yl)amino)-12-oxododecanoate (1.764 g, 2.183 mmol) in DMF (4.0 mL). The mixture was stirred at room temperature for 5 hrs. Solvent was evaporated under reduced pressure to give a residue, which was purified by ISCO (40 g gold column) eluting with 5% MeOH in DCM for 5 column value to remove HOBt followed by 5% to 30% MeOH in DCM to give 1-(((2R,3R,4R,5R,6R)-3-acetamido-4,5-diacetoxy-6-(acetoxymethyl)tetrahydro-2H-pyran-2-yl)oxy)-16,16-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic benzyl ester (3.98 g, 87% yield) as a white solid. ¹H NMR (400 MHz, DMSO-d₆) δ 7.82-7.74 (m, 6H), 7.69 (t, J=5.6 Hz, 3H), 7.33-7.27 (m, 5H), 6.94 (s, 1H), 5.16 (d, J=3.4 Hz, 3H), 5.03 (s, 2H), 4.92 (dd, J=11.2, 3.4 Hz, 3H), 4.43 (d, J=8.4 Hz, 3H), 4.02-3.95 (m, 9H), 3.82 (dt, J=11.2, 8.8 Hz, 3H), 3.65 (dt, J=10.5, 5.6 Hz, 3H), 3.51-3.44 (m, 12H), 3.36 (dt, J=9.6, 6.0 Hz, 3H), 3.01-2.95 (m, 12H), 2.29 (t, J=7.4 Hz, 2H), 2.23 (t, J=6.3 Hz, 6H), 2.05 (s, 9H), 1.99 (t, J=7.0 Hz, 8H), 1.94 (s, 9H), 1.84 (s, 9H), 1.72 (s, 9H), 1.50-1.14 (m, 34H); MS (ESI): 1049.0 (M/2+H)⁺.

### Step 8

To a round bottom flask flushed with Ar was added 10% Pd/C (165 mg, 0.835 mmol) and EtOAc (15 mL). A solution of Benzyl protected tris-GalNAc (1.75 g, 0.835 mmol) in methanol (15 mL) was added followed by triethylsilane (2.67 ml, 16.70 mmol) dropwise. The mixture was stirred at room temperature for 3 hrs. LC-MS showed the reaction was complete, diluted with EtOAc, and filtered through celite, washed with 20% MeOH in EtOAc, concentrated under reduced pressure to give 1-(((2R,3R,4R,5R,6R)-3-acetamido-4,5-diacetoxy-6-(acetoxymethyl)tetrahydro-2H-pyran-2-yl)oxy)-16,16-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic acid (1.67 g, 0.832 mmol, 100% yield) as a white solid. ¹H NMR (400 MHz, DMSO-d₆) δ 11.95 (s, 1H), 7.83-7.74 (m, 6H), 7.69 (t, J=5.7 Hz, 3H), 6.93 (s, 1H), 5.16 (d, J=3.4 Hz, 3H), 4.92 (dd, J=11.2, 3.4 Hz, 3H), 4.43 (d, J=8.4 Hz, 3H), 4.01-3.94 (m, 9H), 3.82 (dt, J=11.3, 8.8 Hz, 3H), 3.66 (dt, J=10.7, 5.6 Hz, 3H), 3.54-3.43 (m, 12H), 3.41-3.33 (m, 3H), 3.03-2.94 (m, 12H), 2.24 (t, J=7.4 Hz, 10H), 2.14 (t, J=7.4 Hz, 2H), 2.06 (s, 9H), 2.00 (t, J=7.2 Hz, 8H), 1.95 (s, 9H), 1.84 (s, 9H), 1.73 (s, 9H), 1.51-1.14 (m, 34H). MS (ESI): 1003.8 (M/2+H)⁺.

### Example 3-2. Synthesis of 22-(((2R,3R,4R,5R,6R)-3-acetamido-4,5-diacetoxy-6-(acetoxymethyl)tetrahydro-2H-pyran-2-yl)oxy)-7,7-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,12,18-trioxo-9-oxa-6,13,17-triazadocosanoic acid

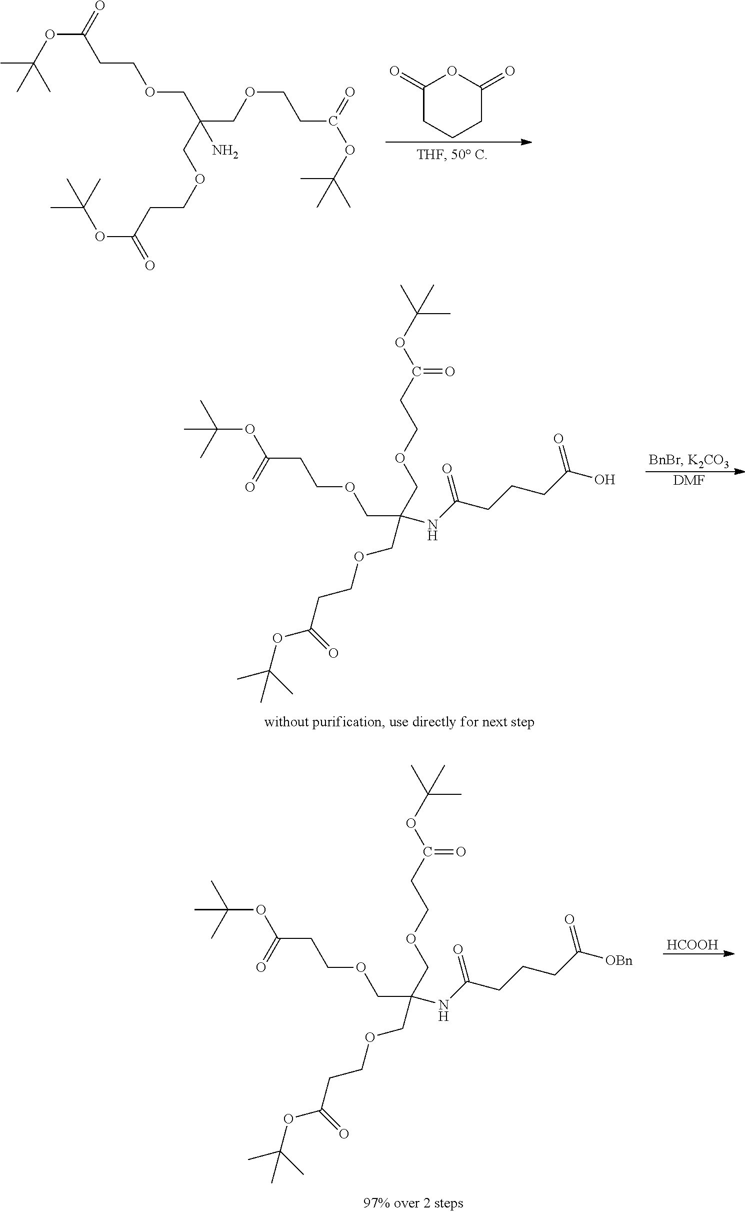

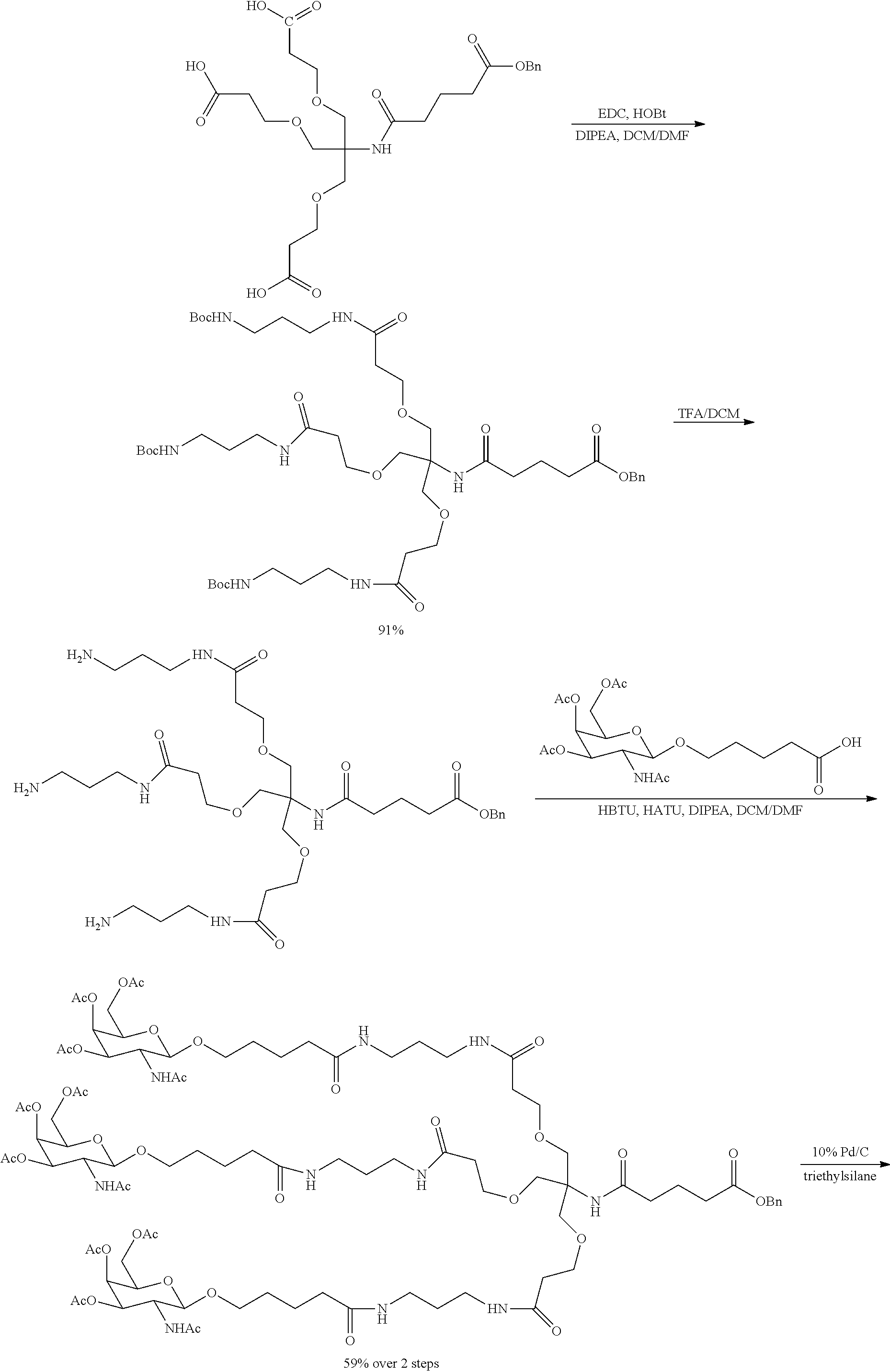

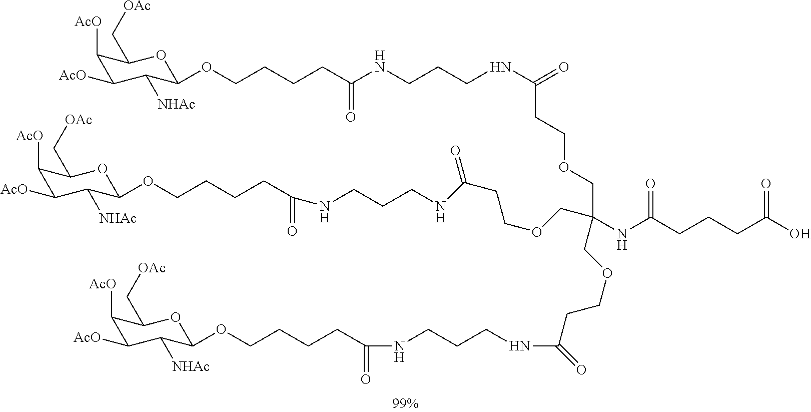 [SMILES: CC(=O)NC1C(OCCCCC(=O)NCCCNC(=O)CCOCC(COCCC(=O)NCCCNC(=O)CCCCOC2OC(COC(C)=O)C(OC(C)=O)C(OC(C)=O)C2NC(C)=O)(COCCC(=O)NCCCNC(=O)CCCCOC2OC(COC(C)=O)C(OC(C)=O)C(OC(C)=O)C2NC(C)=O)NC(=O)CCCC(=O)O)OC(COC(C)=O)C(OC(C)=O)C1OC(C)=O]

### Step 1

A solution of di-tert-butyl 3,3′-((2-amino-2-((3-(tert-butoxy)-3-oxopropoxy)methyl)propane-1,3-diyl)bis(oxy))dipropanoate (4.0 g, 7.91 mmol) and dihydro-2H-pyran-2,6(3H)-dione (0.903 g, 7.91 mmol) in THF (40 mL) was stirred at 50° C. for 3 hrs and at rt for 3 hrs. LC-MS showed desired product. Solvent was evaporated to give the acid, which was directly used for next step without purification.

### Step 2

To a solution of 5-((9-((3-(tert-butoxy)-3-oxopropoxy)methyl)-2,2,16,16-tetramethyl-4,14-dioxo-3,7,11,15-tetraoxaheptadecan-9-yl)amino)-5-oxopentanoic acid (4.90 g, 7.91 mmol) and (bromomethyl)benzene (1.623 g, 9.49 mmol) in DMF was added anhydrous K2CO₃ (3.27 g, 23.73 mmol). The mixture was stirred at 40° C. for 4 hrs and at room temperature for overnight. Solvent was evaporated under reduced pressure. The reaction mixture was diluted with EtOAc, washed with water, dried over anhydrous sodium sulfate, concentrated under reduced pressure to give a residue, which was purified by ISCO eluting with 10% EtOAc in hexane to 50% EtOAc in hexane to give di-tert-butyl 3,3′-((2-(5-(benzyloxy)-5-oxopentanamido)-2-((3-(tert-butoxy)-3-oxopropoxy)methyl)propane-1,3-diyl)bis(oxy))dipropanoate (5.43 g, 7.65 mmol, 97% yield) as a colorless oil. ¹H NMR (400 MHz, Chloroform-d) δ 7.36-7.28 (m, 5H), 6.10 (s, 1H), 5.12 (s, 2H), 3.70 (s, 6H), 3.64 (t, J=8.0 Hz, 6H), 2.50-2.38 (m, 8H), 2.22 (t, J=7.3 Hz, 2H), 1.95 (p, J=7.4 Hz, 2H), 1.45 (s, 27H); MS, 710.5 (M+H)⁺.

### Step 3

A solution of di-tert-butyl 3,3′-((2-(5-(benzyloxy)-5-oxopentanamido)-2-((3-(tert-butoxy)-3-oxopropoxy)methyl)propane-1,3-diyl)bis(oxy))dipropanoate (5.43 g, 7.65 mmol) in formic acid (50 mL) was stirred at room temperature for 48 hrs. LC-MS showed the reaction was not complete. Solvent was evaporated under reduced pressure. The crude product was re-dissolved in formic acid (50 mL) and was stirred at room temperature for 6 hrs. LC-MS showed the reaction was complete. Solvent was evaporated under reduced pressure, co-evaporated with toluene (3×) under reduced pressure, and dried under vacuum to give 3,3′-((2-(5-(benzyloxy)-5-oxopentanamido)-2-((2-carboxyethoxy)methyl)propane-1,3-diyl)bis(oxy))dipropanoic acid (4.22 g, 7.79 mmol, 102% yield) as a white solid. ¹H NMR (500 MHz, DMSO-d₆) δ 12.11 (s, 3H), 7.41-7.27 (m, 5H), 6.97 (s, 1H), 5.07 (s, 2H), 3.55 (t, J=6.4 Hz, 6H), 3.53 (s, 6H), 2.40 (t, J=6.3 Hz, 6H), 2.37-2.26 (m, 2H), 2.08 (t, J=7.3 Hz, 2H), 1.70 (p, J=7.4 Hz, 2H); MS, 542.3 (M+H)⁺.

### Step 4

A solution of 3,3′-((2-(5-(benzyloxy)-5-oxopentanamido)-2-((2-carboxyethoxy)methyl)propane-1,3-diyl)bis(oxy))dipropanoic acid (4.10 g, 7.57 mmol) and HOBt (4.60 g, 34.1 mmol) in DCM (60 mL) and DMF (15 mL) at 0° C. was added tert-butyl (3-aminopropyl)carbamate (5.94 g, 34.1 mmol), EDAC HCl salt (6.53 g, 34.1 mmol) and DIPEA (10.55 ml, 60.6 mmol). The reaction mixture was stirred at 0° C. for 15 minutes and at room temperature for 20 hrs. LC-MS showed the reaction was not complete. EDAC HCl salt (2.0 g) and tert-butyl (3-aminopropyl)carbamate (1.0 g) was added into the reaction mixture. The reaction mixture was stirred at room temperature for 4 hrs. Solvent was evaporated to give a residue, which was dissolved in EtOAc (300 mL), washed with water (1×), saturated sodium bicarbonate (2×), 10% citric acid (2×) and water, dried over sodium sulfate, and concentrated to give a residue which was purified by ISCO (80 g gold cartridge) eluting with DCM to 30% MeOH in DCM to give benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazahenicosan-21-oate (6.99 g, 6.92 mmol, 91% yield) as a white solid. ¹H NMR (500 MHz, Chloroform-d) δ 7.38-7.33 (m, 5H), 6.89 (brs, 3H), 6.44 (s, 1H), 5.23 (brs, 3H), 5.12 (s, 2H), 3.71-3.62 (m, 12H), 3.29 (q, J=6.2 Hz, 6H), 3.14 (q, J=6.5 Hz, 6H), 2.43 (dt, J=27.0, 6.7 Hz, 8H), 2.24 (t, J=7.2 Hz, 2H), 1.96 (p, J=7.5 Hz, 2H), 1.64-1.59 (m, 6H), 1.43 (d, J=5.8 Hz, 27H); MS (ESI): 1011.5 (M+H)⁺.

### Step 5

To a solution of benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazahenicosan-21-oate (0.95 g, 0.940 mmol) in DCM (5 mL) was added TFA (5 mL). The reaction mixture was stirred at room temperature for 4 hrs. LC-MS showed the reaction was completed. Solvent was evaporated under reduced pressure to give benzyl 5-((1,19-diamino-10-((3-aminopropyl)amino)-3-oxopropoxy)methyl)-5,15-dioxo-8,12-dioxa-4,16-diazanonadecan-10-yl)amino)-5-oxopentanoate as a colorless oil. Directly use for next step without purification.

### Step 6

To a solution of 5-(((2R,3R,4R,5R,6R)-3-acetamido-4,5-diacetoxy-6-(acetoxymethyl)tetrahydro-2H-pyran-2-yl)oxy)pentanoic acid (1.684 g, 3.76 mmol), HBTU (1.246 g, 3.29 mmol) and HOBT (0.052 g, 0.376 mmol) in DCM (40 mL) followed by 10-(5-(benzyloxy)-5-oxopentanamido)-N1,N19-dichloro-10-((3-((3-(chloroammonio)propyl)amino)-3-oxopropoxy)methyl)-5,15-dioxo-8,12-dioxa-4,16-diazanonadecane-1,19-diaminium (0.767 g, 0.940 mmol) in DMF (2.0 mL). The mixture was stirred at room temperature for 5 hrs. Solvent was evaporated under reduced pressure to give a residue, which was purified by ISCO (40 g gold column) eluting with DCM to 30% MeOH in DCM to give 22-(((2R,3R,4R,5R,6R)-3-acetamido-4,5-diacetoxy-6-(acetoxymethyl)tetrahydro-2H-pyran-2-yl)oxy)-7,7-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,12,18-trioxo-9-oxa-6,13,17-triazadocosanoic benzyl ester (1.11 g, 0.556 mmol, 59% yield) as a white solid. MS (ESI): 1000.0 (M/2+H)⁺.

### Step 7

To a round bottom flask flushed with Ar was added 10% Pd/C (100 mg, 0.500 mmol) and EtOAc (10 mL). A solution of 22-(((2R,3R,4R,5R,6R)-3-acetamido-4,5-diacetoxy-6-(acetoxymethyl)tetrahydro-2H-pyran-2-yl)oxy)-7,7-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,12,18-trioxo-9-oxa-6,13,17-triazadocosanoic benzyl ester (1.00 g, 0.500 mmol) in methanol (10 mL) was added followed by triethylsilane (1.599 ml, 10.01 mmol) dropwise. The mixture was stirred at room temperature for 3 hrs. LC-MS showed the reaction was complete, diluted with EtOAc, and filtered through celite, washed with 20% MeOH in EtOAc, concentrated under reduced pressure to give 22-(((2R,3R,4R,5R,6R)-3-acetamido-4,5-diacetoxy-6-(acetoxymethyl)tetrahydro-2H-pyran-2-yl)oxy)-7,7-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,12,18-trioxo-9-oxa-6,13,17-triazadocosan-1-oic acid (0.9433 g, 0.494 mmol, 99% yield) as a white solid. ¹H NMR (500 MHz, DMSO-d₆) δ 7.85-7.78 (m, 6H), 7.72 (t, J=5.7 Hz, 3H), 7.03 (s, 1H), 5.20 (d, J=3.4 Hz, 3H), 4.95 (dd, J=11.2, 3.5 Hz, 3H), 4.47 (d, J=8.3 Hz, 3H), 4.05-3.99 (m, 9H), 3.85 (dt, J=11.0, 8.8 Hz, 3H), 3.69 (dt, J=10.6, 5.8 Hz, 3H), 3.52 (dd, J=12.3, 5.6 Hz, 12H), 3.39 (dt, J=11.2, 6.3 Hz, 3H), 3.02 (p, J=6.3 Hz, 12H), 2.26 (t, J=6.4 Hz, 6H), 2.17 (t, J=7.5 Hz, 2H), 2.11-2.07 (m, 11H), 2.03 (t, J=7.1 Hz, 6H), 1.98 (s, 9H), 1.87 (s, 9H), 1.76 (s, 9H), 1.53-1.18 (m, 20H); MS (ESI): 1909.4 (M+H)⁺.

### Example 3-3. Synthesis of 5-(4-(4-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropyl)amino)-6-(bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropyl)amino)-1,3,5-triazin-2-yl)piperazin-1-yl)-5-oxopentanoic acid

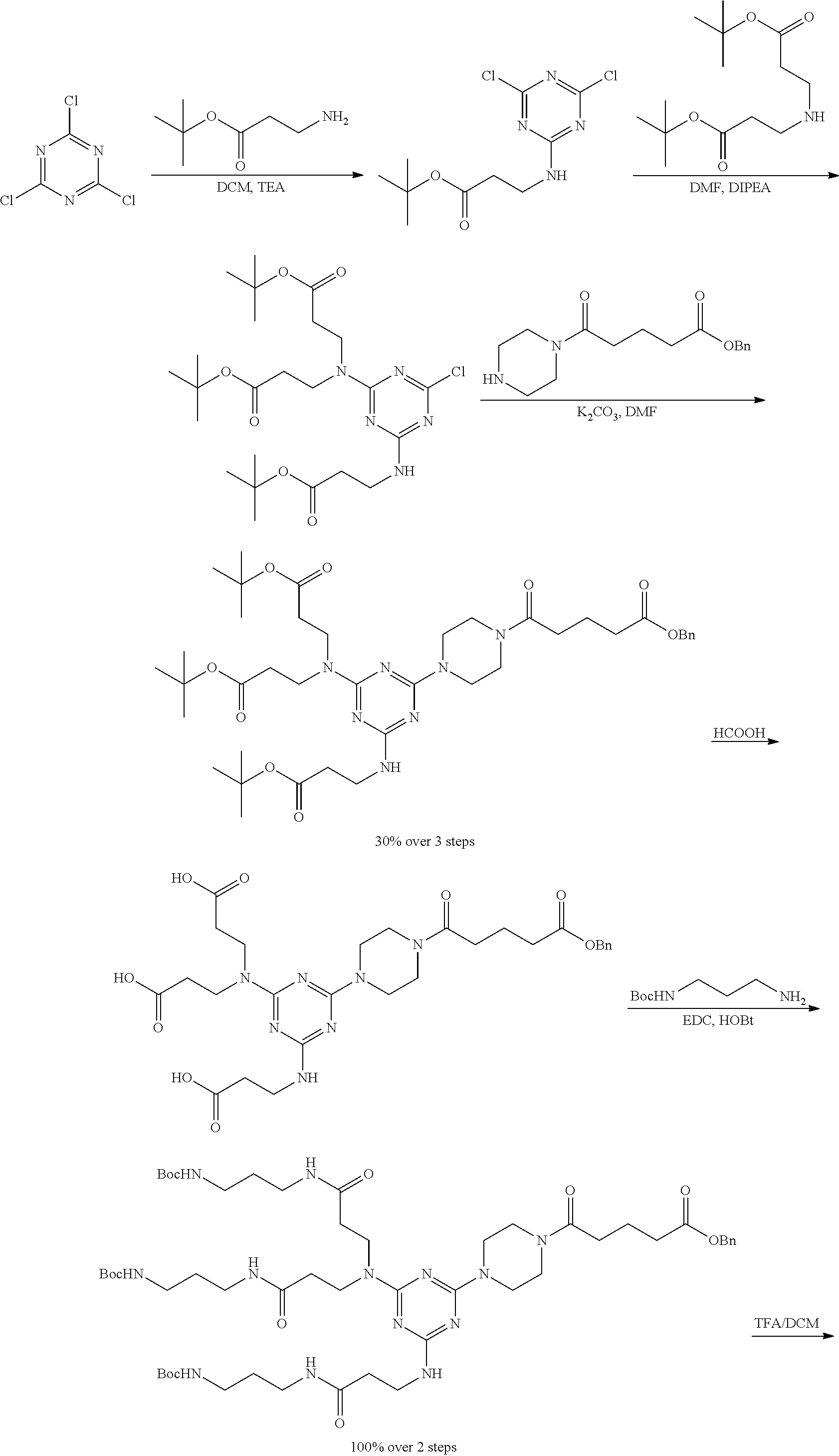

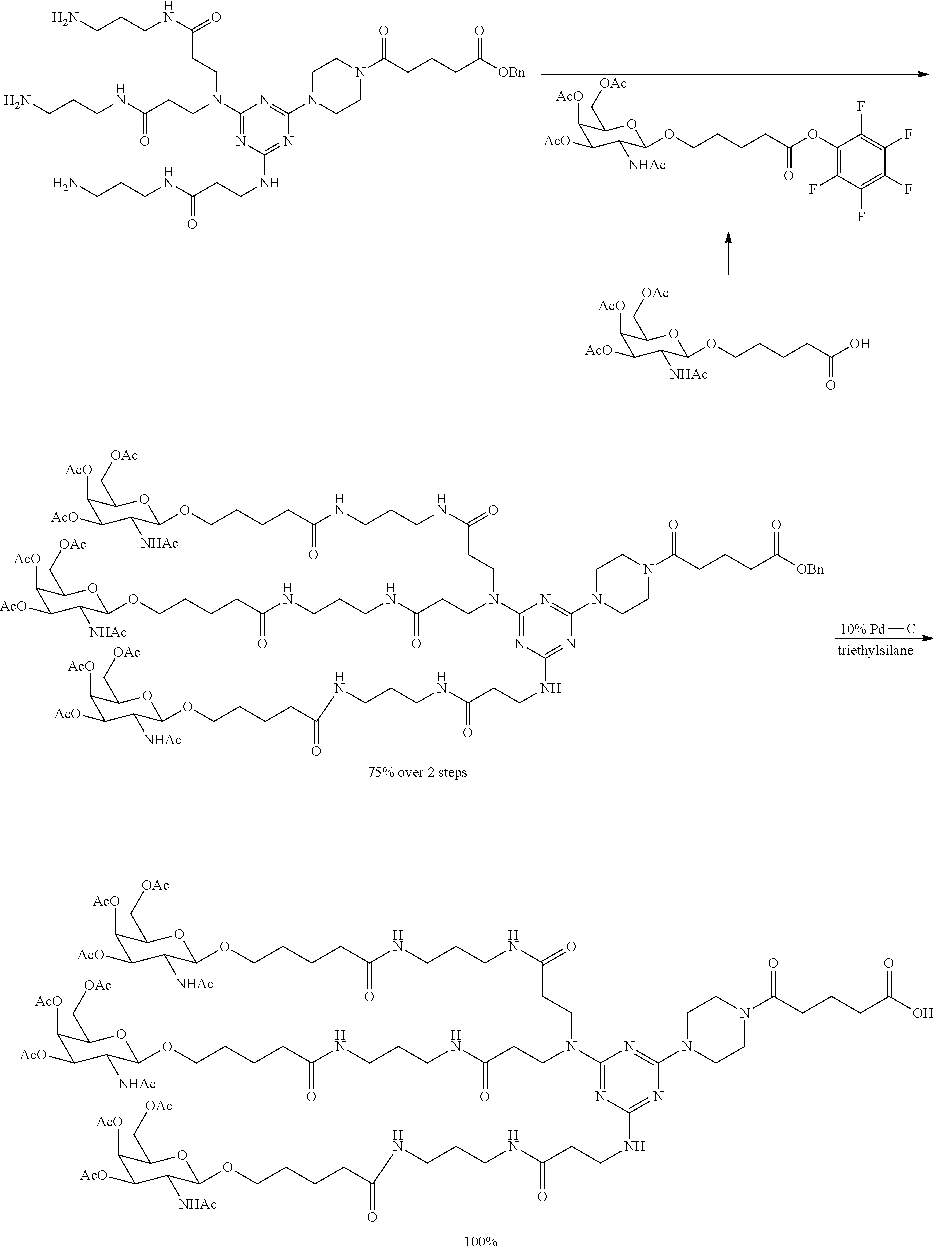

Steps 1 to 3:

To a solid reagent 2,4,6-trichloro-1,3,5-triazine (0.700 g, 3.80 mmol) in DCM (25 mL) at 0° C. was added tert-butyl 3-aminopropanoate HCl salt (0.690 g, 3.80 mmol) and TEA (0.635 ml, 4.56 mmol). The reaction mixture was stirred at 0° C. for 1 hrs. LC-MS showed the desired product. Solvent was evaporated under reduced pressure to give a residue, which was directly used for next step. To a solution of tert-butyl 3-((4,6-dichloro-1,3,5-triazin-2-yl)amino)propanoate (1.114 g, 3.80 mmol) in DMF (15 mL) was added di-tert-butyl 3,3′-azanediyldipropanoate (1.039 g, 3.80 mmol) and DIPEA (1.324 ml, 7.60 mmol). The reaction mixture was stirred at room temperature for 2 hrs. LC-MS showed desired product. To the above reaction mixture was added benzyl 5-oxo-5-(piperazin-1-yl)pentanoate (1.103 g, 3.80 mmol) and K2CO₃ (1.576 g, 11.40 mmol). The reaction mixture was stirred at room temperature for overnight. Diluted with EtOAc, filtered and concentrated under reduced pressure to give a residue, which was purified by ISCO (40 g gold) eluting with 10% EtOAc in hexane to 50% EtOAc in hexane to give di-tert-butyl 3,3′-((4-(4-(5-(benzyloxy)-5-oxopentanoyl)piperazin-1-yl)-6-((3-(tert-butoxy)-3-oxopropyl)amino)-1,3,5-triazin-2-yl)azanediyl)dipropionate (0.90 g, 30%) as a colorless oil. ¹H NMR (500 MHz, Chloroform-d) δ 7.43-7.31 (m, 5H), 5.12 (s, 2H), 3.81-3.66 (m, 8H), 3.60 (dd, J=7.6, 4.8 Hz, 4H), 3.40 (t, J=5.1 Hz, 2H), 2.57-2.44 (m, 8H), 2.39 (t, J=7.4 Hz, 2H), 2.06-1.95 (m, 2H), 1.45 (s, 9H), 1.43 (s, 18H); MS (ESI): 784.7 (M+H)⁺.

### Step 4

A solution of di-tert-butyl 3,3′-((4-(4-(5-(benzyloxy)-5-oxopentanoyl)piperazin-1-yl)-6-43-(tert-butoxy)-3-oxopropyl)amino)-1,3,5-triazin-2-yl)azanediyl)dipropanoate (0.90 g, 1.148 mmol) in formic acid (20 mL) was stirred at room temperature for overnight. LC-MS showed the reaction was not completed and solvent was evaporated. Formic acid (20 mL) was added to the reaction mixture and the reaction mixture was stirred at room temperature for overnight. LC-MS showed the reaction was complete. Solvent was concentrated, co-evaporated with toluene (2×) and dried under vacuum for overnight to give 3,3′-((4-(4-(5-(benzyloxy)-5-oxopentanoyl)piperazin-1-yl)-6-((2-carboxyethyl)amino)-1,3,5-triazin-2-yl)azanediyl)dipropanoic acid (0.75 g, 1.218 mmol, 106% yield) as a white solid. MS (ESI), 616.5 (M+H)⁺.

### Step 5: A solution of 3,3′-((4-(4-(5-(benzyloxy)-5-oxopentanoyl)piperazin-1-yl)-6-((2-carboxyethyl)amino)-1,3,5-triazin-2-yl)azanediyl)dipropanoic acid (0.707 g, 1.148 mmol) and HOBt (0.651 g, 4.82 mmol) in DCM (60 mL) and DMF (15 mL) at 0° C. was added tert-butyl (3-aminopropyl)carbamate (0.840 g, 4.82 mmol), EDAC HCl salt (0.924 g, 4.82 mmol) and DIPEA (1.400 ml, 8.04 mmol). The reaction mixture was stirred at 0° C. for 15 minutes and at room temperature for 20 hrs. LC-MS showed the reaction was not complete. t-Butyl (3-aminopropyl) carbamate (0.28 g) and EDC HCl salt (0.46 g) was added into the reaction mixture. The reaction mixture was continually stirred at room temperature for 4 hrs. Solvent was evaporated to give a residue, which was dissolved in EtOAc (300 mL), washed with water (1×), saturated sodium bicarbonate (2×), 10% citric acid (2×) and water, dried over sodium sulfate, and concentrated to give a residue which was purified by ISCO (80 g gold cartridge) eluting with DCM to 30% MeOH in DCM to give benzyl 5-(4-(4-(bis(3-((3-((tert-butoxycarbonyl)amino)propyl)amino)-3-oxopropyl)amino)-6-((3-((3-((tertbutoxycarbonyl)amino)propyl)amino)-3-oxopropyl)amino)-1,3,5-triazin-2-yl)piperazin-1-yl)-5-oxopentanoate (1.24 g, 1.144 mmol, 100% yield) as a white solid. MS (ESI): 1084.8 (M+H)⁺

### Step 6

A solution of benzyl 5-(4-(4-(bis(3-((3-((tert-butoxycarbonyl)amino)propyl)amino)-3-oxopropyl)amino)-6-((3-((3-((tertbutoxycarbonyl)amino)propyl)amino)-3-oxopropyl)amino)-1,3,5-triazin-2-yl)piperazin-1-yl)-5-oxopentanoate (328.3 mg, 0.303 mmol) in DCM (5.0 mL) was added TFA (3.0 mL). The reaction mixture was stirred at room temperature for 3 hrs. Solvent was evaporated under reduced pressure, use directly for next step without purification. MS (ESI): 784.6 (M+H)⁺.

### Step 7

To a solution of 5-(((2R,3R,4R,5R,6R)-3-acetamido-4,5-diacetoxy-6-(acetoxymethyl)tetrahydro-2H-pyran-2-yl)oxy)pentanoic acid (0.570 g, 1.273 mmol) in DCM (6 mL) was added DIPEA (0.40 mL, 2.296 mmol) and perfluorophenyl 2,2,2-trifluoroacetate (0.535 g, 1.910 mmol). The reaction mixture was stirred at room temperature for 2 hrs. Solvent was evaporated under reduced pressure to give a residue, directly use for next step. MS (ESI): 614.3 (M+H)⁺. A solution of benzyl 5-(4-(4-((3-((3-aminopropyl)amino)-3-oxopropyl)amino)-6-(bis(3-((3-aminopropyl)amino)-3-oxopropyl)amino)-1,3,5-triazin-2-yl)piperazin-1-yl)-5-oxopentanoate (0.238 g, 0.303 mmol) in DCM (15 mL) and DMF (3 mL) was added DIPEA (0.633 ml, 3.64 mmol), and a solution of (2R,3R,4R,5R,6R)-5-acetamido-2-(acetoxymethyl)-6-45-oxo-5-(perfluorophenoxy)pentyl)oxy)tetrahydro-2H-pyran-3,4-diyl diacetate (0.781 g, 1.273 mmol) in DCM (6 mL). The reaction mixture was stirred at room temperature for 4 hrs. Solvent was evaporated under reduced pressure to give a residue, which was purified by ISCO (40 g gold) eluting with DCM to 40% MeOH in DCM to give 5-(4-(4-((3-((3-(5-(((2R,3R,4R,5R,6R)-3-acetamido-4,5-diacetoxy-6-(acetoxymethyl)tetrahydro-2H-pyran-2-yl)oxy)pentanamido) propyl)amino)-3-oxopropyl)amino)-6-(bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropyl)amino)-1,3,5-triazin-2-yl)piperazin-1-yl)-5-oxopentanoic benzyl ester (0.47 g, 0.227 mmol, 74.9% yield) as a white solid. ¹H NMR (500 MHz, DMSO-d₆) δ 7.82-7.78 (m, 6H), 7.70 (t, J=5.7 Hz, 3H), 7.35-7.28 (m, 5H), 6.63 (brs, 1H), 5.20 (d, J=3.3 Hz, 3H), 5.08 (s, 2H), 4.95 (dd, J=11.2, 3.4 Hz, 3H), 4.47 (d, J=8.4 Hz, 3H), 4.05-3.96 (m, 9H), 3.85 (dt, J=11.1, 8.8 Hz, 3H), 3.72-3.53 (m, 12H), 3.43-3.36 (m, 6H), 3.05-2.97 (m, 12H), 2.41-2.27 (m, 10H), 2.08 (s, 9H), 2.03 (d, J=7.0 Hz, 6H), 1.98 (s, 9H), 1.87 (s, 9H), 1.75 (s, 9H), 1.47 (s, 9H), 1.53-1.19 (m, 13H); MS (ESI): 1037.0 (M+H)/2⁺.

### Step 8

To a solution of 5-(4-(4-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropyl)amino)-6-(bis(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropyl)amino)-1,3,5-triazin-2-yl)piperazin-1-yl)-5-oxopentanoic benzyl ester (0.39 g, 0.188 mmol) in EtOAc (10 mL) was added 10% Pd—C(50 mg) followed by 10 mL MeOH under Ar. triethylsilane (0.601 ml, 3.76 mmol) was added to the reaction mixture slowly. The reaction mixture was stirred at room temperature for 2 hrs. filtered through celite, washed with 50% MeOH in EtOAc, solvents were evaporated under reduced pressure to give 5-(4-(4-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropyl)amino)-6-(bis(3-4345-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropyl)amino)-1,3,5-triazin-2-yl)piperazin-1-yl)-5-oxopentanoic acid (0.373 g, 100% yield) a white solid. ¹H NMR (500 MHz, DMSO-d₆) δ 7.82-7.78 (m, 6H), 7.71 (t, J=5.7 Hz, 3H), 6.64 (s, 1H), 5.20 (d, J=3.3 Hz, 3H), 4.95 (dd, J=11.2, 3.4 Hz, 3H), 4.47 (d, J=8.5 Hz, 3H), 4.06-3.96 (m, 9H), 3.85 (dt, J=11.1, 8.8 Hz, 3H), 3.73-3.56 (m, 11H), 3.45-3.35 (m, 5H), 3.09-2.98 (m, 13H), 2.37-2.28 (m, 10H), 2.25 (t, J=7.3 Hz, 2H), 2.09 (s, 9H), 2.03 (t, J=7.0 Hz, 6H), 1.98 (s, 9H), 1.88 (s, 9H), 1.76 (s, 9H), 1.74-1.67 (m, 2H), 1.55-1.40 (m, 15H); MS (ESI): 1983.4 (M+H)⁺.

### Example 4A. Example Compounds for Incorporating Moieties

Synthesis of 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazatetracosan-24-oic acid.

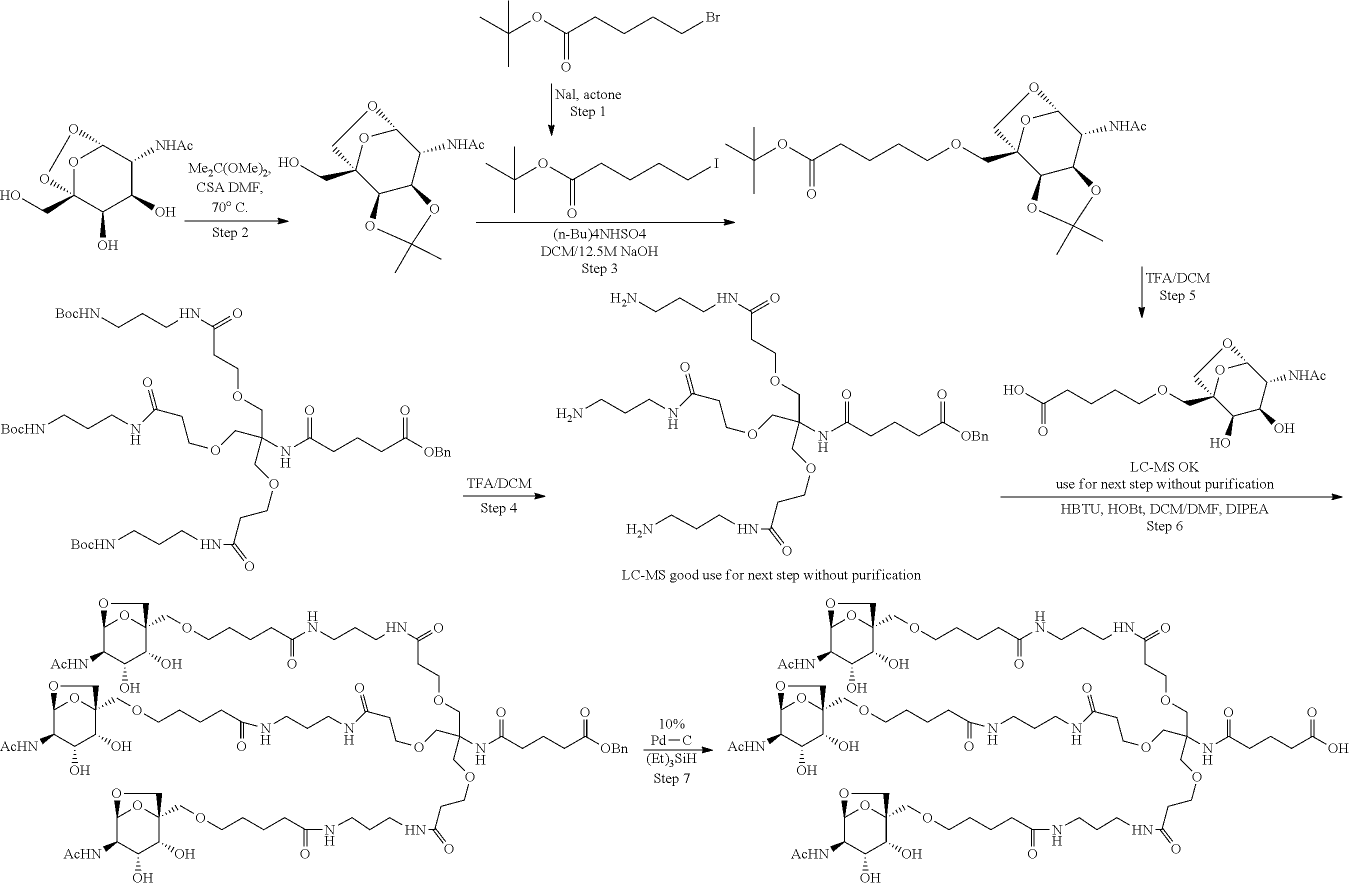

### Step 1

To a solution of tert-butyl 5-bromopentanoate (4.0 g, 16.87 mmol) in acetone (80 mL) was added NaI (7.59 g, 50.6 mmol). The reaction mixture was stirred at 57° C. for 2 hrs, filtered, and washed with EtOAc. Solvent was evaporated under reduced pressure to give a residue, which was dissolved in EtOAc, washed with water, brine, dried over Na₂SO₄, concentrated to give a residue, which was purified by ISCO (40 g column) eluting with 20% EtOAc in hexane to 50% EtOAc in hexane to give tert-butyl 5-iodopentanoate (4.54 g, 15.98 mmol, 95% yield) as a yellow oil. ¹H NMR (500 MHz, Chloroform-d) δ 3.19 (t, J=6.9 Hz, 2H), 2.24 (t, J=7.3 Hz, 2H), 1.86 (p, J=7.1 Hz, 2H), 1.70 (p, J=7.4 Hz, 2H), 1.45 (s, 9H).

### Step 2

To a solution of N-((1S,2R,3R,4R,5S)-2,3-dihydroxy-1-(hydroxymethyl)-6,8-dioxabicyclo[3.2.1]octan-4-yl)acetamide (600 mg, 2.57 mmol) in DMF (15 mL) was added 2,2-dimethoxypropane (2087 μl, 17.03 mmol) followed by (+/−)-camphor-10-sulphonic acid (264 mg, 1.135 mmol). The reaction mixture was stirred at 70° C. for 24 hrs. The reaction mixture was cooled down to room temperature, and then methanol (2.5 mL) was added. The reaction mixture was stirred at room temperature for 30 minutes and neutralized with TEA (0.10 mL). The solvent was evaporated and the residue was coevaporated with toluene. The residue was purified by ISCO (24 g gold) eluting with EtOAc to 10% MeOH in EtOAc to give N-((3aR,4S,7S,8R,8aR)-4-(hydroxymethyl)-2,2-dimethylhexahydro-4,7-epoxy[1,3]dioxolo[4,5-d]oxepin-8-yl)acetamide (666 mg, 2.437 mmol, 95% yield). ¹H NMR (500 MHz, DMSO-d₆) δ 8.09 (d, J=8.1 Hz, 1H), 5.15-5.05 (m, 2H), 4.26 (d, J=5.8 Hz, 1H), 4.09 (dd, J=7.3, 5.8 Hz, 1H), 3.80-3.60 (m, 5H), 1.83 (s, 3H), 1.37 (s, 3H), 1.26 (s, 3H); MS, 274.3 (M+H)⁺.

### Step 3

To a solution of tert-butyl 5-iodopentanoate (1310 mg, 4.61 mmol) and N-((3aR,4S,7S,8R,8aR)-4-(hydroxymethyl)-2,2-dimethylhexahydro-4,7-epoxy[1,3]dioxolo[4,5-d]oxepin-8-yl)acetamide 7 (420 mg, 1.537 mmol) in DCM (10.5 mL) was added tetrabutylammonium hydrogensulfate (783 mg, 2.305 mmol) followed by 12.5 M sodium hydroxide solution (7 mL). The reaction mixture was stirred at room temperature for 24 hrs. The reaction mixture was diluted with DCM and water, extracted with DCM (2×). The organic layer was washed with 1 N HCl solution, and dried over sodium sulfate. Solvent was concentrated under reduce pressure to give a residue. The resulting crude material was added ethyl acetate (30 mL) and sonicated for 5 minutes. The result precipitate was filtered, washed with ethyl acetate (10 mL×2). LC-MS showed the filter does not contain desired product and was tetrabutylammonium salt. The filtrate was concentrated under reduced pressure to give a residue, which was purified by ISCO (40 g silica gel gold cartridge) eluting with 50% EtOAc in hexane to EtOAc to give tert-butyl 5-(((3aR,4S,7S,8R,8aR)-8-acetamido-2,2-dimethylhexahydro-4,7-epoxy[1,3]dioxolo[4,5-d]oxepin-4-yl)methoxy)pentanoate (0.470 g, 1.094 mmol, 71.2% yield) as a yellowish oil. ¹H NMR (500 MHz, Chloroform-d) δ 5.56 (d, J=9.1 Hz, 1H), 4.21 (d, J=5.9 Hz, 1H), 4.12 (dtd, J=7.7, 3.8, 1.7 Hz, 1H), 3.99 (t, J=6.3 Hz, 1H), 3.90 (d, J=9.5 Hz, 1H), 3.77 (d, J=2.0 Hz, 2H), 3.67 (d, J=9.5 Hz, 1H), 3.52 (ddt, J=30.5, 9.2, 5.8 Hz, 2H), 2.23 (t, J=7.1 Hz, 2H), 2.03 (d, J=14.5 Hz, 3H), 1.65-1.55 (m, 7H), 1.44 (s, 9H), 1.35 (s, 3H); MS, 452.4 (M+Na)⁺.

### Step 4

To a solution of benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazahenicosan-21-oate (0.168 g, 0.166 mmol) in DCM (3 mL) was added TFA (3 mL). The reaction mixture was stirred at room temperature for 3 hrs. LC-MS showed the reaction was completed. Solvent was evaporated under reduced pressure to give benzyl 5-((1,19-diamino-10-((3-aminopropyl)amino)-3-oxopropoxy)methyl)-5,15-dioxo-8,12-dioxa-4,16-diazanonadecan-10-yl)amino)-5-oxopentanoate as a colorless oil. MS, 710.5 (M+H)+. Directly use for next step without purification.

### Step 5

To a solution of tert-butyl 5-(((3aR,4S,7S,8R,8aR)-8-acetamido-2,2-dimethylhexahydro-4,7-epoxy[1,3]dioxolo[4,5-d]oxepin-4-yl)methoxy)pentanoate (285 mg, 0.664 mmol) in DCM (5 mL) was added TFA (5 mL) was stirred at room temperature for 4 hrs. LC-MS showed the reaction was complete. Solvent was evaporated to give 5-(((1S,2R,3R,4R,5S)-4-acetamido-2,3-dihydroxy-6,8-dioxabicyclo[3.2.1]octan-1-yl)methoxy)pentanoic acid. MS (ESI): 334.3 (M+H)+. Directly use for next step without purification.

### Step 6

To a solution of 5-(((1S,2R,3R,4R,5S)-4-acetamido-2,3-dihydroxy-6,8-dioxabicyclo[3.2.1]octan-1-yl)methoxy)pentanoic acid (221 mg, 0.664 mmol) in DCM (10 mL) was added DIPEA (2313 μl, 13.28 mmol), HBTU (208 mg, 0.548 mmol), HOBT (67.3 mg, 0.498 mmol), a solution of benzyl 5-((1,19-diamino-10-((3-aminopropyl)amino)-3-oxopropoxy)methyl)-5,15-dioxo-8,12-dioxa-4,16-diazanonadecan-10-yl)amino)-5-oxopentanoate (118 mg, 0.166 mmol) (GL08-02) in DMF (3.0 mL) and DCM (5.0 mL). The reaction mixture was stirred at room temperature for overnight. LC-MS showed the desired product. Solvent was evaporated under reduced pressure to give a residue, which was purified by ISCO (24 g gold cartridge) eluting with DCM to 80% MeOH in DCM to give benzyl 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazatetracosan-24-oate (272 mg, 0.164 mmol, 99% yield) (product @ tube 30 to 42 (40% MeOH in DCM to 60% MeOH in DCM)). ¹H NMR (500 MHz, DMSO-d₆) δ 7.89 (d, J=7.8 Hz, 3H), 7.81 (t, J=5.7 Hz, 3H), 7.75 (s, 3H), 7.34 (q, J=7.5, 6.9 Hz, 5H), 7.05 (s, 1H), 5.07 (s, 5H), 4.83 (d, J=5.3 Hz, 3H), 4.56 (d, J=7.1 Hz, 3H), 3.73 (dd, J=23.3, 9.2 Hz, 6H), 3.64 (d, J=7.0 Hz, 6H), 3.58-3.35 (m, 27H), 3.02 (p, J=6.2 Hz, 12H), 2.33 (t, J=7.6 Hz, 2H), 2.26 (t, J=6.4 Hz, 6H), 2.10 (t, J=7.6 Hz, 2H), 2.04 (t, J=7.4 Hz, 6H), 1.82 (s, 9H), 1.72 (q, J=7.6 Hz, 2H), 1.52-1.39 (m, 18H); MS (ESI), 1656.3 (M+H)⁺.

### Step 7

To a solution of benzyl 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazatetracosan-24-oate (270 mg, 0.163 mmol) in EtOAc (10 mL) was added 10% Pd—C (50 mg), and MeOH (5.0 mL), and triethylsilane (1042 μl, 6.52 mmol). The reaction mixture was stirred at room temperature for 1 hr, filtered, and concentrated to give 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazatetracosan-24-oic acid (246 mg, 0.157 mmol, 96% yield) as a white solid. ¹H NMR (500 MHz, DMSO-d₆) δ 11.99 (brs, 1H), 7.89 (d, J=7.9 Hz, 3H), 7.82 (t, J=5.4 Hz, 3H), 7.75 (t, J=5.7 Hz, 3H), 7.03 (s, 1H), 5.07 (d, J=1.6 Hz, 3H), 4.83 (brs, 3H), 4.56 (brs, 3H), 3.79-3.68 (m, 6H), 3.64 (d, J=7.2 Hz, 6H), 3.58-3.34 (m, 27H), 3.02 (p, J=6.3 Hz, 12H), 2.27 (t, J=6.4 Hz, 6H), 2.17 (t, J=7.5 Hz, 2H), 2.08 (t, J=7.5 Hz, 2H), 2.04 (t, J=7.3 Hz, 6H), 1.82 (s, 9H), 1.65 (p, J=7.5 Hz, 2H), 1.54-1.40 (m, 18H); MS(ESI), 1566.3 (M+H)+.

### Example 4B

Synthesis of 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazahentriacontan-31-oic acid

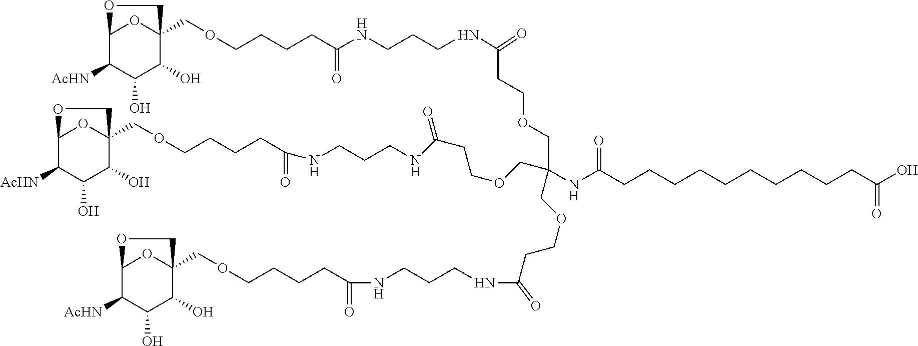 [SMILES: CC(=O)N[C@H]1[C@H]2OC[C@](COCCCCC(=O)NCCCNC(=O)CCOCC(COCCC(=O)NCCCNC(=O)CCCCOC[C@@]34CO[C@@H](O3)[C@H](NC(C)=O)[C@@H](O)[C@H]4O)(COCCC(=O)NCCCNC(=O)CCCCOC[C@@]34CO[C@@H](O3)[C@H](NC(C)=O)[C@@H](O)[C@H]4O)NC(=O)CCCCCCCCCCC(=O)O)(O2)[C@H](O)[C@@H]1O]

18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazahentriacontan-31-oic acid was synthesized using the same procedure as 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazatetracosan-24-oic acid. ¹H NMR (400 MHz, DMSO-d₆) δ 7.90 (d, J=7.8 Hz, 3H), 7.83 (t, J=5.7 Hz, 3H), 7.76 (t, J=5.7 Hz, 3H), 6.98 (d, J=6.2 Hz, 1H), 5.09 (s, 3H), 3.81-3.69 (m, 6H), 3.69-3.62 (m, 6H), 3.62-3.40 (m, 24H), 3.04 (p, J=6.1 Hz, 9H), 2.28 (t, J=6.4 Hz, 4H), 2.18 (t, J=7.3 Hz, 2H), 2.06 (t, J=7.7 Hz, 6H), 1.84 (s, 6H), 1.48 (tq, J=14.9, 7.4 Hz, 16H), 1.23 (s, 8H). MS(ESI), 1664.0 (M+H)⁺.

### Example 5. Example Compounds for Incorporating Moieties

### Synthesis of 5-(4-(4,6-bis((3-43-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropyl)amino)-1,3,5-triazin-2-yl)piperazin-1-yl)-5-oxopentanoic acid

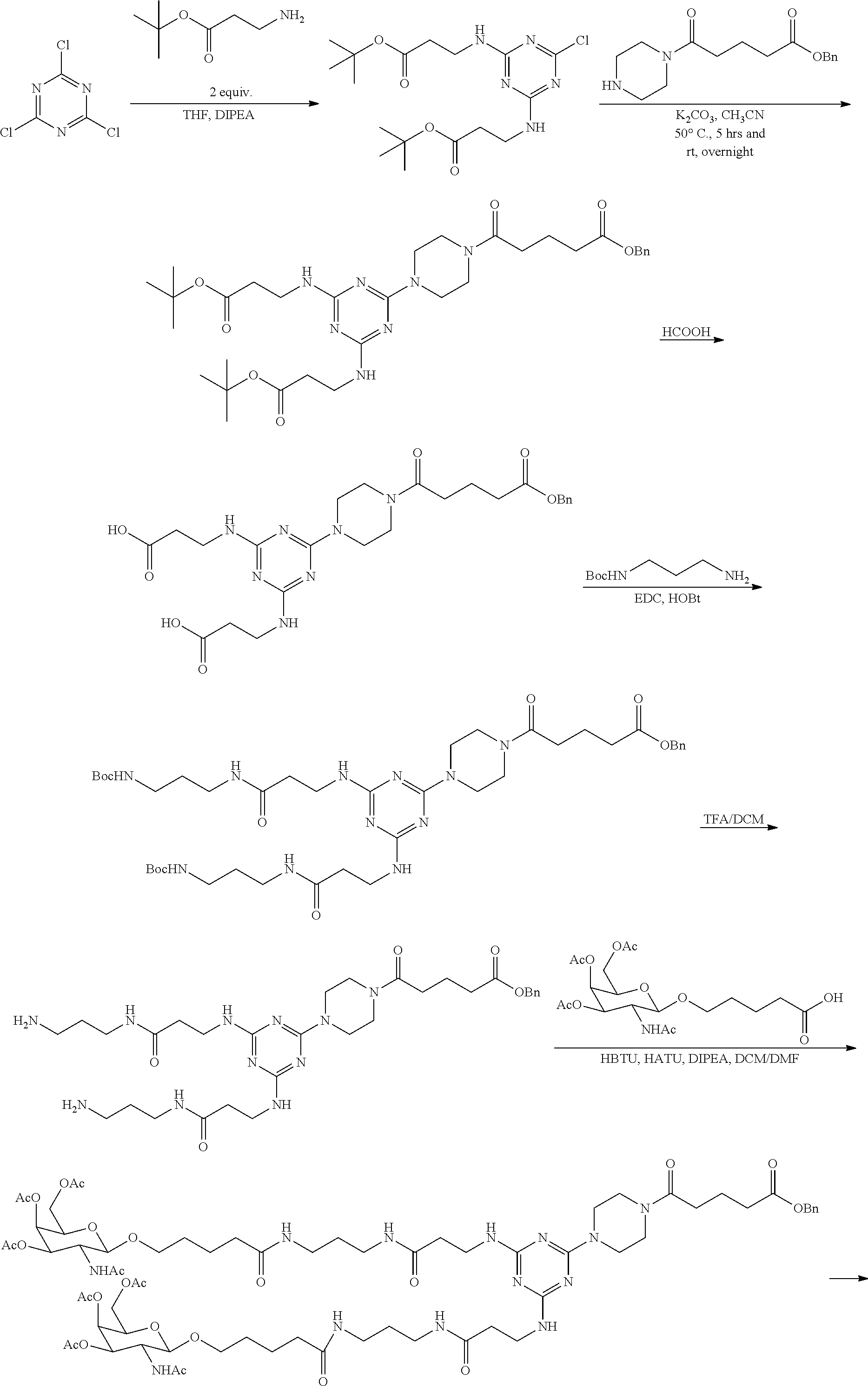

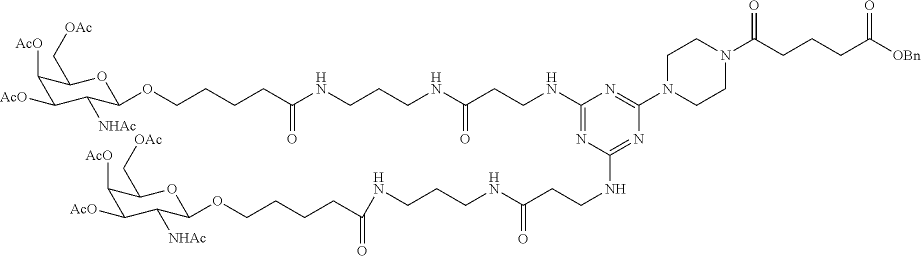 [SMILES: CC(=O)NC1C(OCCCCC(=O)NCCCNC(=O)CCNc2nc(NCCC(=O)NCCCNC(=O)CCCCOC3OC(COC(C)=O)C(OC(C)=O)C(OC(C)=O)C3NC(C)=O)nc(N3CCN(C(=O)CCCC(=O)OCc4ccccc4)CC3)n2)OC(COC(C)=O)C(OC(C)=O)C1OC(C)=O]

Steps 1 to 2:

To a solid reagent 2,4,6-trichloro-1,3,5-triazine (0.500 g, 2.71 mmol) in THF (30 mL) was added tert-butyl 3-aminopropanoate HCl salt (0.985 g, 5.42 mmol) and DIPEA (2.36 ml, 13.56 mmol). The reaction mixture was stirred at room temperature for 5 hrs. LC-MS showed the desired product; MS(ESI): 402.4 (M+H)⁺. Solvent was evaporated under reduced pressure to give a residue, which was directly used for next step. To a solution of di-tert-butyl 3,3′-((6-chloro-1,3,5-triazine-2,4-diyl)bis(azanediyl))dipropionate (1.052 g, 2.71 mmol) in acetonitrile (50 mL) was added benzyl 5-oxo-5-(piperazin-1-yl)pentanoate (1.103 g, 3.80 mmol) and K2CO₃ (2.248 g, 16.27 mmol). The reaction mixture was stirred at room temperature for overnight and at 50° C. Diluted with EtOAc, filtered and concentrated under reduced pressure to give a residue, which was purified by ISCO (40 g gold) eluting with 20% EtOAc in hexane to 50% EtOAc in hexane to give di-tert-butyl 3,3′-((6-(4-(5-(benzyloxy)-5-oxopentanoyl)piperazin-1-yl)-1,3,5-triazine-2,4-diyl)bis(azanediyl))dipropionate (1.13 g, 64%) as a white solid. ¹H NMR (400 MHz, Chloroform-d) δ 7.43-7.30 (m, 5H), 5.15 (s, 2H), 3.75 (brs, 4H), 3.63 (brs, 6H), 3.43 (brs, 2H), 2.51 (q, J=7.0, 6.5 Hz, 6H), 2.42 (t, J=7.4 Hz, 2H), 2.09-1.96 (m, 2H), 1.48 (s, 18H); MS (ESI): 656.6 (M+H)⁺.

### Step 3

A solution of di-tert-butyl 3,3′-((6-(4-(5-(benzyloxy)-5-oxopentanoyl)piperazin-1-yl)-1,3,5-triazine-2,4-diyl)bis(azanediyl))dipropionate (1.10 g, 1.68 mmol) in formic acid (20 mL) was stirred at room temperature for overnight. LC-MS showed the reaction was not completed and solvent was evaporated. Formic acid (20 mL) was added to the reaction mixture and the reaction mixture was stirred at room temperature for 5 hrs. LC-MS showed the reaction was complete. Solvent was concentrated, co-evaporated with toluene (2×) and dried under vacuum for overnight to give 3,3′-((6-(4-(5-(benzyloxy)-5-oxopentanoyl)piperazin-1-yl)-1,3,5-triazine-2,4-diyl)bis(azanediyl))dipropionic acid (0.91 g, 100% yield) as a white solid. MS (ESI), 544.2 (M+H)⁺.

### Step 4

A solution of 3,3′-((6-(4-(5-(benzyloxy)-5-oxopentanoyl)piperazin-1-yl)-1,3,5-triazine-2,4-diyl)bis(azanediyl))dipropionic acid (0.91 g, 1.68 mmol) and HOBt (0.76 g, 4.36 mmol) in DCM (30 mL) and DMF (3 mL) at 0° C. was added tert-butyl (3-aminopropyl)carbamate (0.840 g, 4.36 mmol), EDC HCl salt (0.836 g, 4.36 mmol) and DIPEA (1.460 ml, 8.39 mmol). The reaction mixture was stirred at 0° C. for 15 minutes and at room temperature for 20 hrs. Solvent was evaporated to give a residue, which was dissolved in EtOAc (300 mL), washed with water (1×), saturated sodium bicarbonate (2×), 10% citric acid (2×) and water, dried over sodium sulfate, and concentrated to give a residue which was purified by ISCO (80 g gold cartridge) eluting with DCM to 30% MeOH in DCM to give benzyl 5-(4-(4,6-bis((3-((tert-butoxycarbonyl)amino)propyl)amino)-3-oxopropyl)amino)-1,3,5-triazin-2-yl)piperazin-1-yl)-5-oxopentanoate (1.11 g, 77% yield) as a white solid. MS (ESI): 857.5 (M+H)⁺.

### Step 5

A solution of benzyl 5-(4-(4,6-bis((3-((3-((tert-butoxycarbonyl)amino)propyl)amino)-3-oxopropyl)amino)-1,3,5-triazin-2-yl)piperazin-1-yl)-5-oxopentanoate (212.3 mg, 0.250 mmol) in DCM (5.0 mL) was added TFA (3.0 mL). The reaction mixture was stirred at room temperature for 3 hrs. Solvent was evaporated under reduced pressure, use directly for next step without purification. MS (ESI): 656.3 (M+H)⁺.

### Step 6

To a solution of 5-(((2R,3R,4R,5R,6R)-3-acetamido-4,5-diacetoxy-6-(acetoxymethyl)tetrahydro-2H-pyran-2-yl)oxy)pentanoic acid (333 mg, 0.740 mmol) in DCM (5 mL) was added DIPEA (2.16 ml, 12.4 mmol), HBTU (235 mg, 0.620 mmol), HOBT (67 mg, 0.50 mmol), a solution of benzyl 5-(4-(4,6-bis((3-((3-aminopropyl)amino)-3-oxopropyl)amino)-1,3,5-triazin-2-yl)piperazin-1-yl)-5-oxopentanoate (163 mg, 0.250 mmol) in DCM (3.0 mL). The reaction mixture was stirred at room temperature for 3 hrs. LC-MS showed the desired product. Solvent was evaporated under reduced pressure to give a residue, which was purified by ISCO (24 g gold cartridge) eluting with DCM to 50% MeOH in DCM to give (2R,2′R,3R,3′R,4R,4′R,5R,5′R,6R,6′R)-((((((3,3′-((6-(4-(5-(benzyloxy)-5-oxopentanoyl) piperazin-1-yl)-1,3,5-triazine-2,4-diyl)bis(azanediyl))bis(propanoyl))bis(azanediyl))bis(propane-3,1-diyl))bis(azanediyl))bis(5-oxopentane-5,1-diyl))bis(oxy))bis(5-acetamido-2-(acetoxymethyl)tetrahydro-2H-pyran-6,3,4-triyl)tetraacetate (460 mg) containing some HOBt. MS (ESI), 1515.7 (M+H)⁺.

### Step 7

To a solution of (2R,2′R,3R,3′R,4R,4′R,5R,5′R,6R,6′R)-((((((3,3′-((6-(4-(5-(benzyloxy)-5-oxopentanoyl)piperazin-1-yl)-1,3,5-triazine-2,4-diyl)bis(azanediyl))bis(propanoyl))bis(azanediyl))bis(propane-3,1-diyl))bis(azanediyl))bis(5-oxopentane-5,1-diyl))bis(oxy))bis(5-acetamido-2-(acetoxymethyl)tetrahydro-2H-pyran-6,3,4-triyl)tetraacetate (0.44 g, 0.290 mmol) in EtOAc (20 mL) was added 10% Pd—C(40 mg) followed by 2.0 mL MeOH under Ar. Triethylsilane (2.784 ml, 17.43 mmol) was added to the reaction mixture slowly. The reaction mixture was stirred at room temperature for 2 hrs, filtered through celite, washed with 50% MeOH in EtOAc. Solvents were evaporated under reduced pressure to give 5-(4-(4,6-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropyl)amino)-1,3,5-triazin-2-yl)piperazin-1-yl)-5-oxopentanoic acid (0.43 g, 100% yield) a white solid. MS (ESI): 1425.0 (M+H)⁺.

### Example 6. Example Compounds for Incorporating Moieties

Synthesis of 5-(4-(4,6-bis((3-((3-(5-(((1S,2R,3R,4R,5S)-4-acetamido-2,3-dihydroxy-6,8-dioxabicyclo[3.2.1]octan-1-yl)methoxy)pentanamido)propyl)amino)-3-oxopropyl)amino)-1,3,5-triazin-2-yl)piperazin-1-yl)-5-oxopentanoic acid.

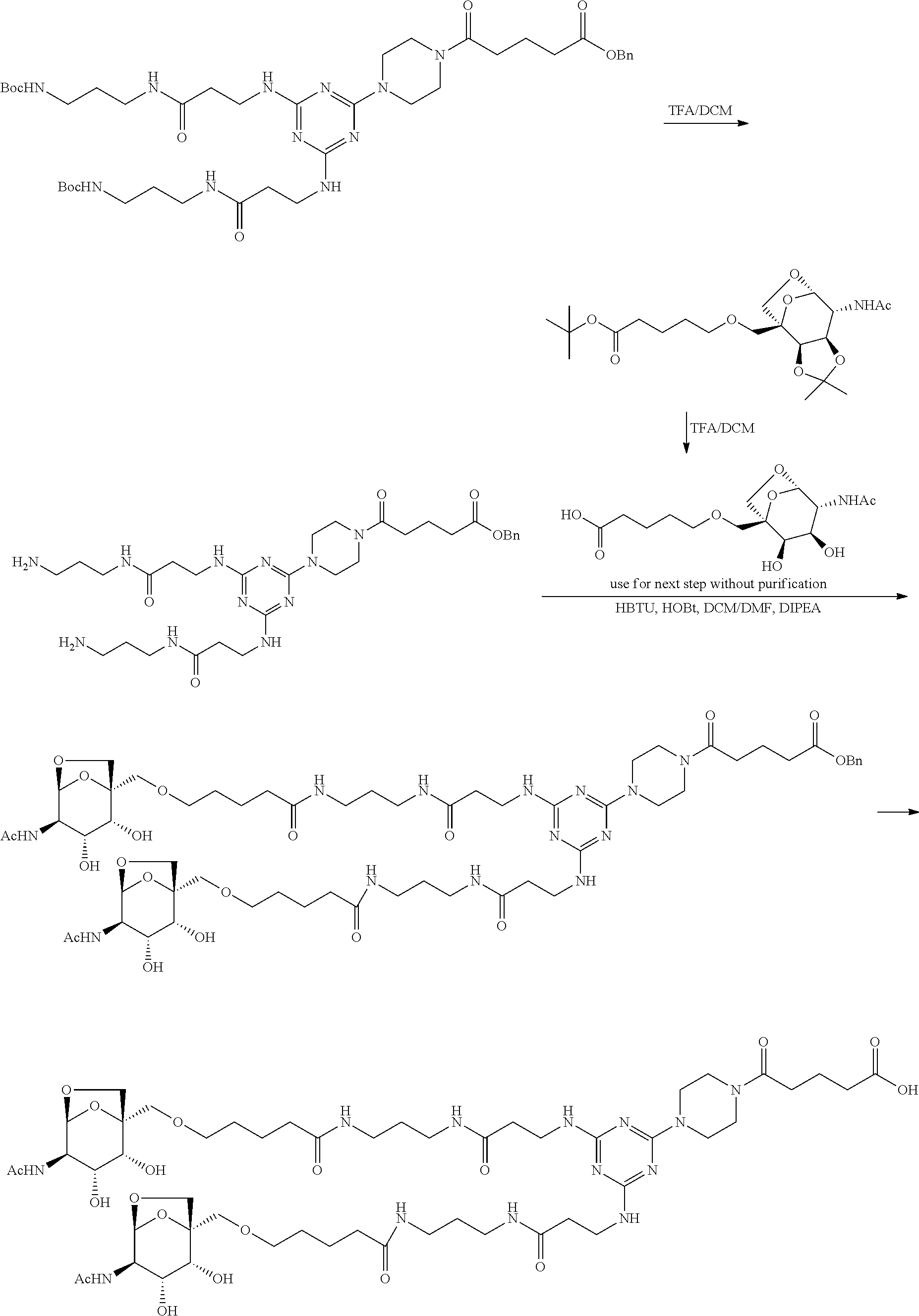

### Step 1

A solution of benzyl 5-(4-(4,6-bis((3-((3-((tert-butoxycarbonyl)amino)propyl)amino)-3-oxopropyl)amino)-1,3,5-triazin-2-yl)piperazin-1-yl)-5-oxopentanoate (212.3 mg, 0.250 mmol) in DCM (5.0 mL) was added TFA (3.0 mL). The reaction mixture was stirred at room temperature for 3 hrs. Solvent was evaporated under reduced pressure, use directly for next step without purification. MS (ESI): 656.3 (M+H)⁺.

### Step 2

To a solution of tert-butyl 5-(((3aR,4S,7S,8R,8aR)-8-acetamido-2,2-dimethylhexahydro-4,7-epoxy[1,3]dioxolo[4,5-d]oxepin-4-yl)methoxy)pentanoate (373 mg, 0.870 mmol) in DCM (5 mL) was added TFA (5 mL) was stirred at room temperature for 4 hrs. LC-MS showed the reaction was complete. Solvent was evaporated to give 5-(((1S,2R,3R,4R,5S)-4-acetamido-2,3-dihydroxy-6,8-dioxabicyclo[3.2.1]octan-1-yl)methoxy)pentanoic acid. MS (ESI): 334.3 (M+H)+. Directly use for next step without purification.

### Step 3

To a solution of 55-(((1S,2R,3R,4R,5S)-4-acetamido-2,3-dihydroxy-6,8-dioxabicyclo[3.2.1]octan-1-yl)methoxy)pentanoic acid (289 mg, 0.870 mmol) in DCM (5 mL) was added DIPEA (2.16 ml, 12.4 mmol), HBTU (330 mg, 0.870 mmol), HOBT (67 mg, 0.50 mmol), a solution of benzyl 5-(4-(4,6-bis((3-((3-aminopropyl)amino)-3-oxopropyl)amino)-1,3,5-triazin-2-yl)piperazin-1-yl)-5-oxopentanoate (163 mg, 0.250 mmol) in DCM (3.0 mL). The reaction mixture was stirred at room temperature for 3 hrs. LC-MS showed the desired product. Solvent was evaporated under reduced pressure to give a residue, which was purified by ISCO (24 g gold cartridge) eluting with DCM to 50% MeOH in DCM to give benzyl 5-(4-(4,6-bis((3-((3-(5-(((1S,2R,3R,4R,5S)-4-acetamido-2,3-dihydroxy-6,8-dioxabicyclo[3.2.1]octan-1-yl)methoxy)pentanamido)propyl)amino)-3-oxopropyl)amino)-1,3,5-triazin-2-yl)piperazin-1-yl)-5-oxopentanoate (227 mg, 71%). MS (ESI), 1287.0 (M+H)⁺.

### Step 4

To a solution of benzyl 5-(4-(4,6-bis((3-((3-(5-(((1S,2R,3R,4R,5S)-4-acetamido-2,3-dihydroxy-6,8-dioxabicyclo[3.2.1]octan-1-yl)methoxy)pentanamido)propyl)amino)-3-oxopropyl)amino)-1,3,5-triazin-2-yl)piperazin-1-yl)-5-oxopentanoate (0.167 g, 0.130 mmol) in EtOAc (10 mL) was added 10% Pd—C(50 mg) followed by 2.0 mL MeOH under Ar. Triethylsilane (1.66 ml, 10.39 mmol) was added to the reaction mixture slowly. The reaction mixture was stirred at room temperature for 2 hrs, filtered through celite, washed with 50% MeOH in EtOAc. Solvents were evaporated under reduced pressure to give 5-(4-(4,6-bis((3-((3-(5-(((1S,2R,3R,4R,5S)-4-acetamido-2,3-dihydroxy-6,8-dioxabicyclo[3.2.1]octan-1-yl)methoxy)pentanamido)propyl)amino)-3-oxopropyl)amino)-1,3,5-triazin-2-yl)piperazin-1-yl)-5-oxopentanoic acid (32 mg, 21% yield) a white solid. MS (ESI): 1196.7 (M+H)⁺.

### Example 7. Example Preparation of Certain Phosphoramidites

In some embodiments, the present disclosure provides monomers (phosphoramidites) and methods thereof for oligonucleotide preparation. In some embodiments, provided phosphoramidites comprise 5′-end structures that provides special and/or greatly improved activities and/or properties. In some embodiments, provided phosphoramidites comprise desired chemical moieties, e.g., carbohydrate moieties, lipid moieties, etc., for incorporation into oligonucleotides. In some embodiments, provided phosphoramidites comprise linkers/handles for incorporation of desired chemical moieties, e.g., carbohydrate moieties, lipid moieties, etc. Many technologies can be utilized to prepare phosphoramidites in accordance with the present disclosure, including but not limited to those described in WO/2010/064146, WO/2011/005761, WO/2013/012758, WO/2014/010250, US2013/0178612, WO/2014/012081, WO/2015/107425, WO/2017/015555, and WO/2017/062862, the methods and reagents of each of which are incorporated herein by reference. Provided below as examples are preparation of certain phosphoramidites.

### Example 7-1. Preparation of Thymidine-5′-dimethylvinylphosphonate-2′-deoxy-3′—CNE Phosphoramidite

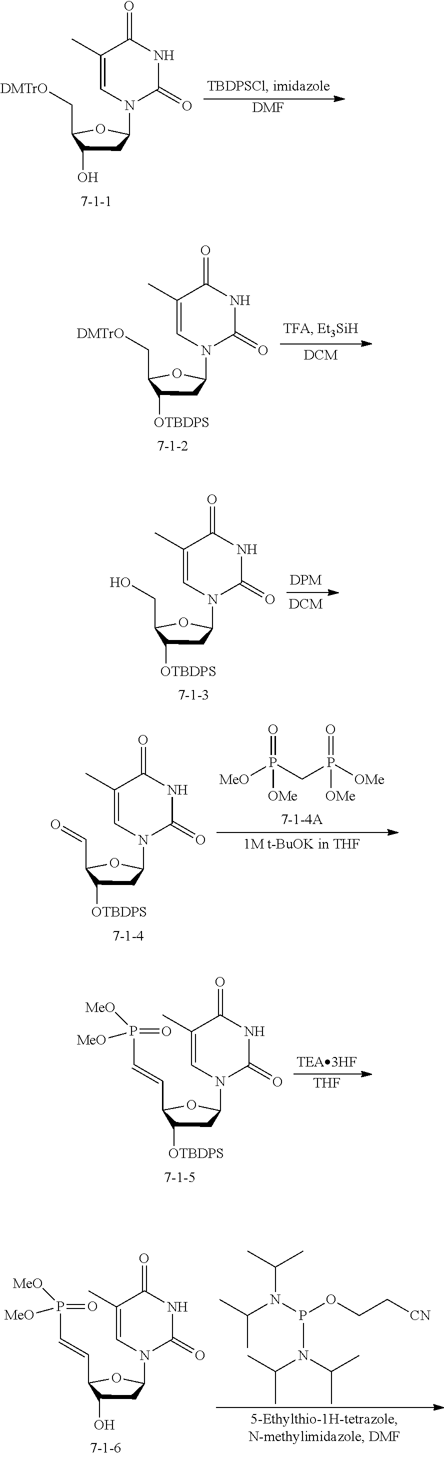

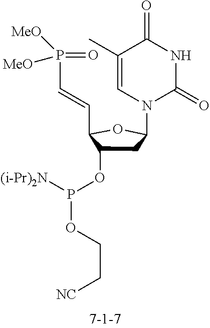

### Preparation of Compound 7-1-2

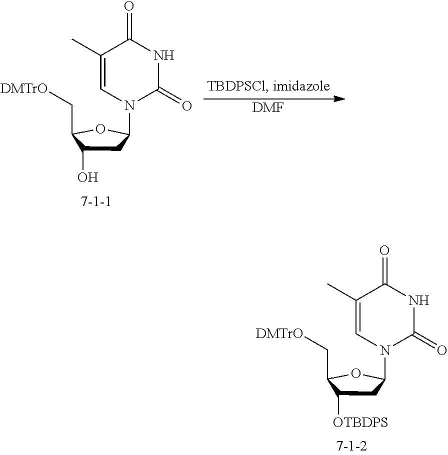

To a solution of compound 7-1-1 (20.00 g, 36.72 mmol, 1.00 eq.) in DMF (100.00 mL) was added imidazole (25.00 g, 367.20 mmol, 10.00 eq.) followed by TBDPSCl (50.47 g, 183.60 mmol, 47.17 mL, 5.00 eq.). The reaction mixture was stirred at 25° C. for 16 h. TLC (Dichloromethane: Methanol=1:1) showed compound 7-1-1 was consumed completely. EtOAc (300 mL) was added and the mixture was washed with water (60 mL*3). The organic phase was dried over Na₂SO₄, filtered and concentrated. The residue was purified by column chromatography on silica gel (Petroleum ether:Ethyl acetate=20:1, 1:1, 1:4). Compound 7-1-2 (30.00 g) was obtained as white foamy solid. ¹H NMR: (CDCl₃, 400 MHz) δ=8.165 (s, 1H), 7.575-7.080 (m, 21H), 6.718-6.741 (m, 4H), 6.473 (d, J=2.8 Hz, 1H), 4.520-4.534 (m, 1H), 4.037-4.043 (d, J=2.4 Hz, 1H), 3.758 (s, 6H), 3.184-3.217 (m, 1H), 2.841-2.874 (m, 1H), 2.319-2.338 (m, 1H), 2.025-2.078 (m, 1H), 1.321 (s, 3H), 1.021 (s, 9H).

### Preparation of Compound 7-1-3

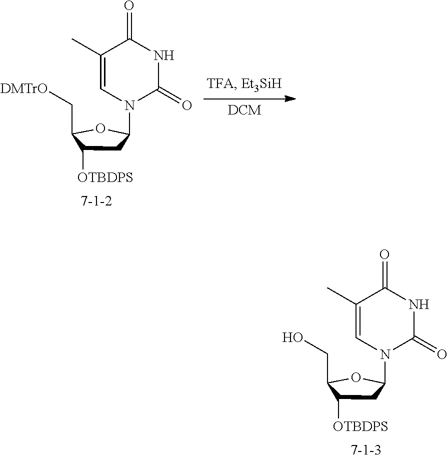

To a solution of compound 7-1-2 (25.00 g, 31.93 mmol, 1.00 eq.) in DCM (250 mL) was added TFA (8.37 g, 73.44 mmol, 5.44 mL, 2.30 eq.). The color of the solution turned to red. Et₃SiH (8.17 g, 70.24 mmol, 11.19 mL, 2.20 eq.) was added at 25° C. The reaction mixture was stirred at 25° C. for 2 h and the red solution became colorless. TLC (Petroleum ether:Ethyl acetate=1:1) showed compound 7-1-2 was consumed completely. The solvent was removed under reduced pressure, and the residue was dissolved in EtOAc (100 mL). The organic phase was washed with NaHCO₃ (40 mL), brine, dried over Na₂SO₄, filtered and concentrated. The residue was purified by column chromatography on silica gel (Petroleum ether:Ethyl acetate=20:1, 1:1). Compound 7-1-3 (9.80 g, 56.20% yield, 88% purity) was obtained as white solid. ¹H NMR: (CDCl₃, 400 MHz) δ=8.108 (s, 1H), 7.643 (s, 1H), 7.403-7.412 (m, 6H), 7.269 (d, J=4.8 Hz, 2H), 6.217 (d, J=5.6 Hz, 1H), 4.451 (s, 1H), 3.975 (s, 1H), 3.631 (d, J=12 Hz, 1H), 3.255 (s, 1H), 2.264-2.296 (m, 1H), 2.136-2.184 (m, 1H), 1.957 (s, 1H), 1.859 (s, 3H), 1.090 (s, 9H).

### Preparation of Compound 7-1-4

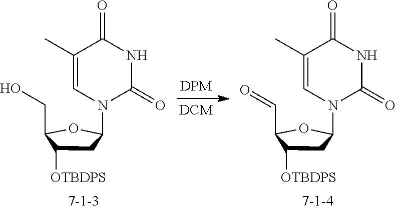

To a solution of compound 7-1-3 (18.00 g, 37.45 mmol, 1.00 eq.) in DCM (500 mL) was added DMP (17.47 g, 41.20 mmol, 12.75 mL, 1.10 eq.) at 0° C. The reaction mixture was stirred at 25° C. for 3 h. TLC (Petroleum ether:Ethyl acetate=1:1) showed the reaction was complete. Na₂SO₃ (sat., 100 mL) and NaHCO₃ (sat.100 mL) was added successively. The mixture was extracted with DCM (100 mL*3). The organic phase was dried over Na₂SO₄ and concentrated. Compound 7-1-4 (17.92 g, crude) was obtained as yellow oil.

### Preparation of Compound 7-1-5

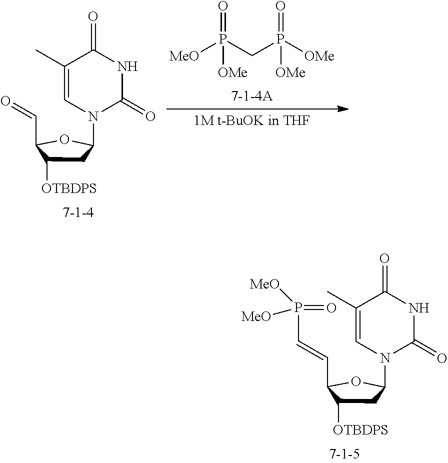

To a solution of compound 7-1-4A (16.08 g, 69.26 mmol, 1.85 eq.) in THF (29 mL) was added t-BuOK (1 M, 69.26 mL, 1.85 eq.) at 0° C. The mixture was stirred at 0° C. for 10 min, then warmed up to 25° C. for 30 min. The above mixture was added to a solution of compound 7-1-4 (17.92 g, 37.44 mmol, 1.00 eq.) in THF (36 mL) at 0° C. The reaction mixture was stirred at 0° C. for 1 h and then allowed to warm up to 25° C. in 80 min. TLC (Dichloromethane: Methanol=20:1) showed the reaction was complete. To the reaction mixture water (200 mL) was added and extracted with EtOAc (300 mL*4). The organic phase was dried (Na₂SO₄), filtered and concentrated. The residue was purified by column chromatography on silica gel (PE (10% DCM): EA=10:1, 1:8). Compound 7-1-5 (15.00 g) was obtained as yellow solid.

### Preparation of Compound 7-1-6

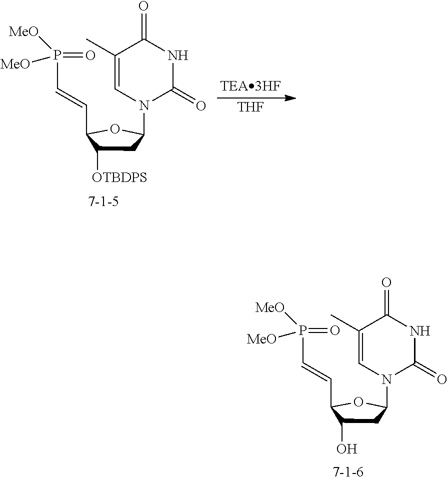

To a solution of compound 7-1-5 (21.00 g, 35.92 mmol, 1.00 eq.) in THF (60 mL) was added N, N-diethylethanamine; trihydrofluoride (28.95 g, 179.59 mmol, 29.24 mL, 5.00 eq.) at 25° C. The reaction mixture was stirred at 25° C. for 20 h. TLC (Dichloromethane: Methanol=10:1) showed the reaction was complete. The reaction mixture was concentrated under reduced pressure and the mixture was neutralized with Na₂CO₃ (aq., sat) until pH=7. The water phase was freeze-dried. The freeze-drying solid was washed with DCM: MeOH=10:1(300 mL*2). The organic phase was concentrated. The residue obtained was purified by column chromatography on silica gel (Dichloromethane: Methanol=100:1,100:8). Compound 7-1-6 (5.20 g, 15.02 mmol, 41.81% yield) was obtained as white solid. ¹H NMR: (CDCl₃, 400 MHz) δ=9.521 (s, 1H), 7.120 (s, 1H), 6.974-7.074 (m, 1H), 6.372-6.405 (m, 1H), 5.961-6.050 (m, 1H), 4.684 (s, 1H), 4.504-4.518 (m, 1H), 4.393-4.409 (m, 1H), 3.726-3.775 (m, 6H), 3.151-3.180 (m, 2H), 2.411-2.427 (m, 1H), 1.930-2.218 (m, 1H), 1.927 (s, 3H).

### Preparation of Compound 7-1-7

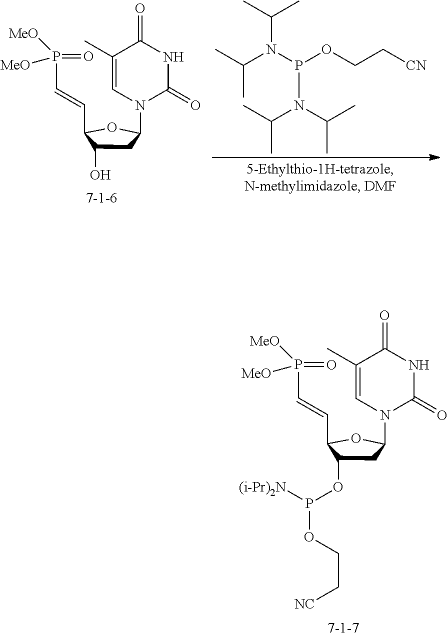

To a solution of compound 7-1-6 (3.80 g, 10.97 mmol, 1.00 eq.) in DMF (23 mL) was added 5-ethylsulfanyl-2H-tetrazole (1.43 g, 10.97 mmol, 1.00 eq.), 1-methylimidazole (1.80 g, 21.94 mmol, 1.75 mL, 2.00 eq.) and 3-bis(diisopropylamino)phosphanyloxypropanenitrile (4.96 g, 16.46 mmol, 5.22 mL, 1.50 eq.). The reaction mixture was stirred at 25° C. under N2 for 3 h. TLC (Dichloromethane: Methanol=10:1) showed the reaction was complete. The reaction mixture was diluted with EtOAc (200 mL). The reaction mixture was washed with aq. saturated. NaHCO₃ solution (20 mL*4), dried over Na₂SO₄, filtered and concentrated under reduced pressure. The column was eluted with MeOH (20 min), EA (20 min), Petroleum ether (20 min), and Petroleum ether/Ethyl acetate (20 min). The residue thus obtained was purified by silica gel column chromatography (elution with Petroleum ether: EtOAc=10:1, 1:1 and then EtOAc/Acetonitrile=1000:1,100:2,100:4). Compound 7-1-7 (4.80 g, 8.78 mmol, 80.04% yield) was obtained as yellow solid. MS: LCMS, Calculated C₂₂H36N408P2, 546.2008; Observed in +Ve mode 568.95; 569.43[M+Na]. ¹H NMR: (CDCl₃, 400 MHz) δ=9.489 (s, 1H), 7.233 (s, 1H), 6.835-7.035 (m, 1H), 6.303-6.337 (m, 1H), 5.931-5.983 (m, 1H), 4.388-4.504 (m, 1H), 3.703-3.846 (m, 1H), 3.666-3.694 (m, 6H), 3.533-3.559 (m, 2H), 2.594-2.702 (m, 2H), 2.162-2.578 (m, 2H), 1.863 (s, 3H), 1.111-1.189 (m, 12H). ¹³C NMR (101 MHz, CDCl₃) δ 163.66, 162.54, 150.47, 150.40, 148.68, 148.61, 148.41, 148.35, 135.10, 135.01, 118.73, 118.25, 117.76, 117.61, 116.91, 116.85, 116.38, 111.74, 84.83, 84.79, 84.75, 84.72, 84.62, 84.56, 84.53, 84.50, 84.40, 84.33, 77.40, 77.29, 77.09, 76.77, 76.03, 75.87, 75.49, 75.48, 75.34, 75.32, 58.21, 58.19, 58.16, 58.12, 58.00, 57.92, 52.59, 52.55, 52.54, 52.52, 52.49, 52.46, 45.33, 45.27, 43.43, 43.40, 43.30, 43.27, 38.45, 38.40, 38.37, 36.45, 24.62, 24.57, 24.54, 24.49, 24.46, 22.96, 22.94, 22.88, 22.85, 20.47, 20.39, 20.37, 20.30, 20.11, 20.04, 12.50, 12.48. ³¹P NMR (162 MHz, CDCl₃) δ 149.40, 149.38, 19.99, 19.64, 14.10.

### Example 7-2. Stereopure L-DPSE-5′-DMT-5′VP-dT Amidite, 7-2-8

### Preparation of L-DPSE-NOPCl

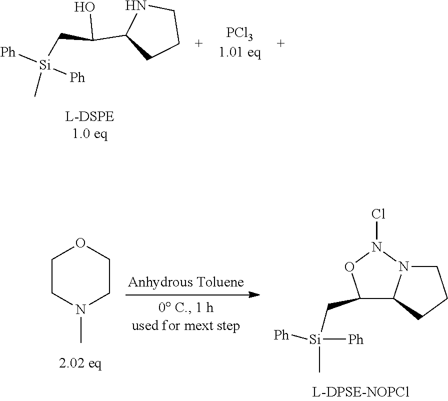

L-DPSE (8.82 g, 28.5 mmol) was dried by azeotropic evaporation with anhydrous toluene (60 ml) at 35° C. in a rotary evaporator and further dried in high vacuum for overnight. A solution of this dried L-DPSE and 4-methylmorpholine (5.82 g, 6.33 mL, 57.5 mmol) which was dissolved in anhydrous toluene (50 ml) was added to a solution of PCl₃ (4.0 g, 2.5 mL, 29.0 mmol) in anhydrous toluene (25 ml) placed in 250 mL three neck round bottomed flask which was cooled at −5° C. under argon (start Temp: −2° C., Max: 5° C. temp, 10 min addition) and the reaction mixture was stirred at 0° C. for another 40 min. After that the precipitated white solid was filtered by vacuum under argon using special filter tube (Chemglass: Medium Frit, Airfree, Schlenk). The solvent was removed by rotary evaporator under argon at bath temperature (25° C.) and the crude oily mixture was obtained and dried under vacuum overnight (˜15 h) and used for next step.

### Preparation of L-DPSE-5′-DMT-5′VP-dT Amidite

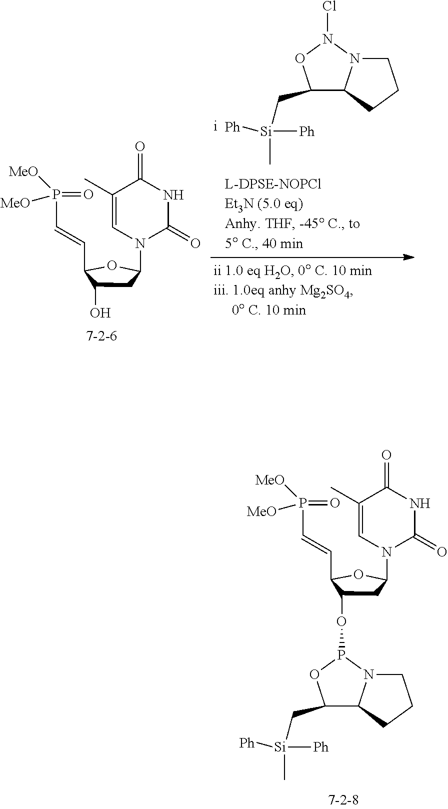

Compound 7-2-6 (7.0 g, 20.2 mmol) was dried two times by co-evaporation with 75 mL of anhydrous toluene at 45° C. and kept at high vacuum for overnight. Then the dried Compound 7-2-6, was dissolved in dry THF (70 mL) in a 250 mL three neck flasks under argon, followed by the addition of triethylamine (14 mL, 101 mmol) and the mixture was cooled to −45° C. To this cooled reaction mixture was added a solution of the crude L-DPSE-NOPCl (28.5 mmol, 1.4 eq, in THF 50 mL) from the previous step via syringe dropwise (˜10 min, maintaining the internal temperature −40 to −35° C.). The reaction mixture was then gradually warmed to 5° C. After 30 min at 5° C., TLC and LC-MS analysis indicated the complete conversion of SM to product (total reaction time 2 h). The reaction mixture was cooled in an ice bath, and was quenched by addition of water (0.36 mL, 20.2 mmol) and stirred for 10 min followed by added anhydrous Mg₂SO₄ (3.0 g, 20.2 mmol). The reaction was filtered through Airfree, Schlenk filter tube, washed with dry THF (50 mL) and evaporated under rotary evaporation at 28° C. to afford the pale-yellow solid of the crude product, which was dried under high vacuum for overnight. The dried crude product was purified by 120 silica column (which was pre-deactivated with 3 column volume of ethyl acetate with 5% TEA) using ethyl acetate/hexane mixture with 5% TEA as a solvent. After column purification, fractions were analyzed by TLC and LC-MS and pooled together. Solvent was evaporated in a rotary evaporator at 28° C. and the residue was dried under high vacuum to afford the product as a white solid. Yield: 11.8 g (87%). ¹H NMR (400 MHz, Chloroform-d) δ 7.46 (ddt, J=16.5, 7.6, 2.7 Hz, 4H), 7.33-7.17 (m, 6H), 6.93-6.88 (m, 1H), 6.75 (ddd, J=22.6, 17.2, 4.4 Hz, 1H), 6.16 (dd, J=7.5, 6.3 Hz, 1H), 5.85 (ddd, J=19.2, 17.1, 1.8 Hz, 1H), 4.71 (dt, J=8.7, 5.7 Hz, 1H), 4.38 (dp, J=10.7, 3.6 Hz, 1H), 4.15 (tt, J=5.6, 2.7 Hz, 1H), 3.68 (dd, J=11.1, 3.7 Hz, 6H), 3.55-3.29 (m, 2H), 3.09 (tdd, J=10.8, 8.8, 4.3 Hz, 1H), 2.11 (ddd, J=13.9, 6.3, 3.3 Hz, 1H), 1.96 (s, 1H), 1.87 (d, J=1.2 Hz, 3H), 1.85-1.73 (m, 2H), 1.70-1.49 (m, 2H), 1.38 (ddd, J=15.9, 10.4, 6.3 Hz, 2H), 1.26-1.11 (m, 2H), 0.60 (s, 3H). ³¹P NMR (162 MHz, CDCl₃) δ 152.41, 19.95. ¹³C NMR (101 MHz, CDCl₃) δ 171.07, 163.62, 163.59, 150.21, 150.19, 148.49, 148.43, 136.61, 135.84, 135.15, 134.57, 134.33, 129.48, 129.42, 127.97, 127.93, 127.81, 118.38, 116.50, 111.52, 85.02, 84.72, 84.70, 84.51, 84.48, 79.25, 79.16, 77.40, 77.28, 77.08, 76.76, 74.93, 74.91, 74.83, 74.81, 68.01, 67.98, 60.35, 52.60, 52.55, 52.47, 52.42, 47.03, 46.67, 38.12, 38.08, 27.18, 25.85, 25.82, 21.01, 17.58, 17.54, 14.19, 12.58,-3.00,-3.27. MS: LCMS, Calculated C₃₂H41N3O8P2Si, 685.7255: Observed in +Ve mode: 686.21 [M+H], 708.14 [M+Na].

### Example 7-3. Synthesis of 5′-DMT-2′OMe-5-Lipid-3′—CNE Phosphoramidite —Incorporation of Desired Moieties Through Nucleobases

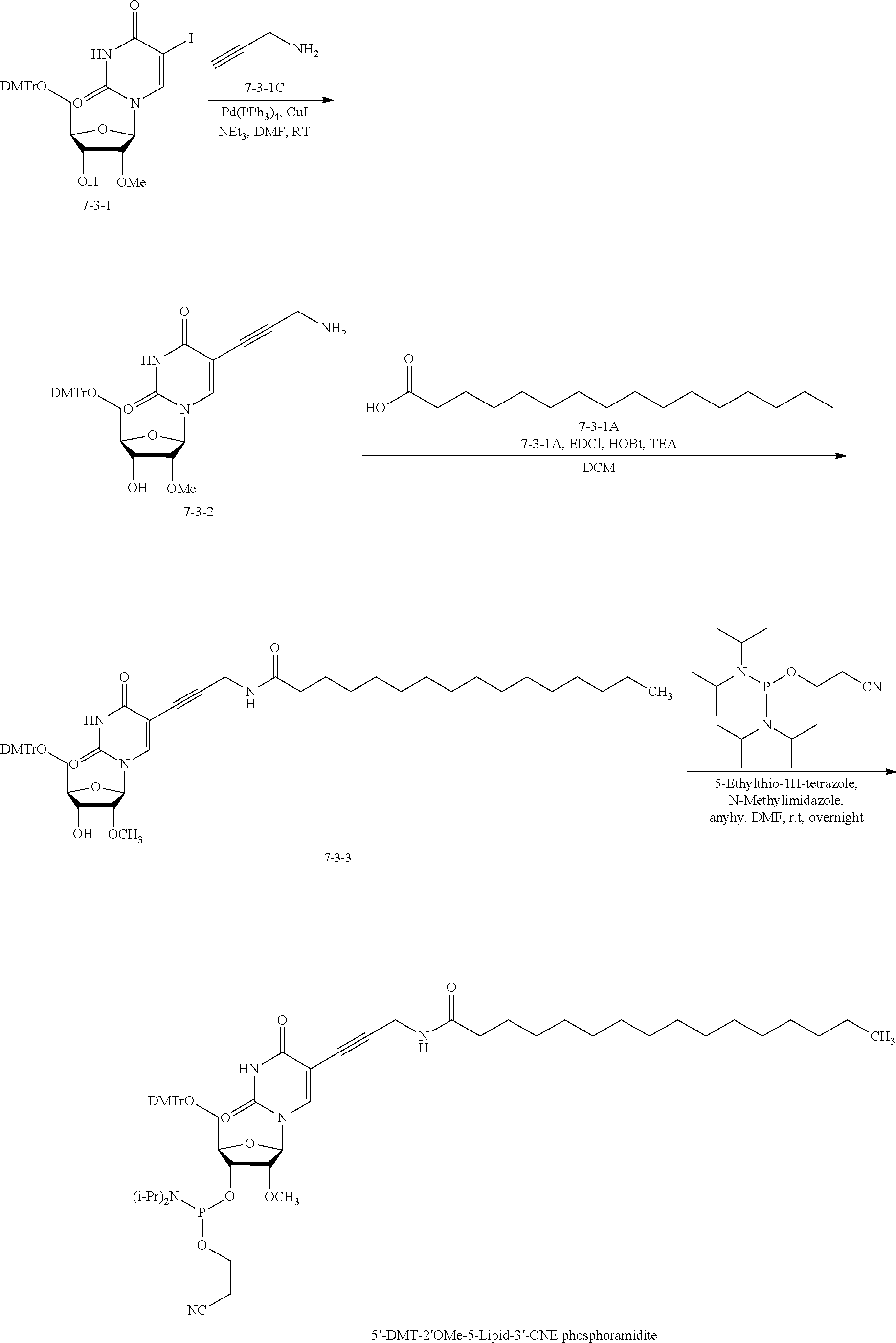

### Preparation of Compound 7-3-2

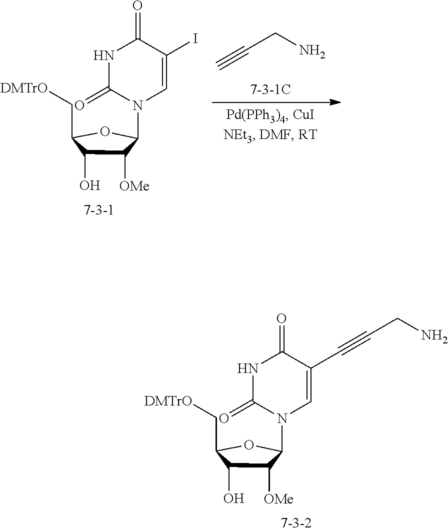

A mixture of compound 7-3-1 (13.00 g, 18.94 mmol), prop-2-yn-1-amine (2.09 g, 37.87 mmol, 2.43 mL), CuI (901.63 mg, 4.73 mmol), Pd(PPh₃)₄ (2.19 g, 1.89 mmol) and TEA (3.83 g, 37.87 mmol, 5.25 mL) in DMF (130 mL) was degassed and purged with N2 for 3 times, and then the mixture was stirred at 25° C. for 12 hour under N2 atmosphere and dark. LC-MS showed Compound 7-3-1 was consumed completely and one main peak with desired MS was detected. The mixture was concentrated in vacuo. The residue was purified by column chromatography (SiO₂, Dichloromethane/Methanol=100/1 to 0:1). Compound 7-3-2 (11.00 g, crude) was obtained as a yellow solid. ¹H NMR (400 MHz, CDCl₃): δ=8.23 (s, 1H), 7.48-7.14 (m, 13H), 6.83 (br d, J=7.3 Hz, 5H), 5.94 (br s, 1H), 4.48 (br t, J=5.8 Hz, 2H), 4.05 (br d, J=6.4 Hz, 2H), 3.93 (br d, J=2.9 Hz, 1H), 3.81-3.70 (m, 8H), 3.62 (s, 4H), 3.52 (br d, J=11.0 Hz, 2H), 3.35 (br d, J=9.0 Hz, 1H). LCMS: (M+H⁺): 614.2. TLC (Dichloromethane/Methanol=10:1) Rf=0.19.

### Preparation of Compound 7-3-3

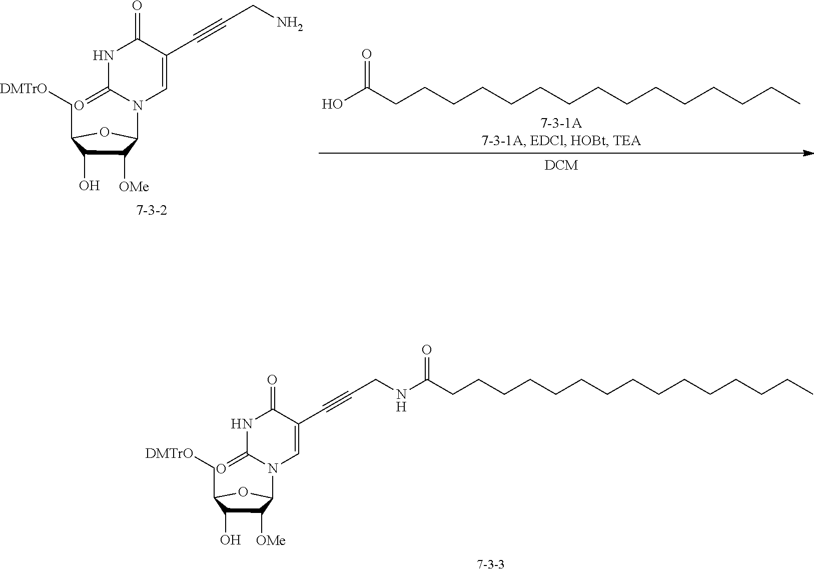

To a solution of palmitic acid (5.06 g, 19.72 mmol) in DCM (130 mL) was added TEA (3.63 g, 35.85 mmol, 4.97 mL), EDCI (5.15 g, 26.89 mmol), HOBt (3.63 g, 26.89 mmol), and Compound 7-3-3 (11.00 g, 17.93 mmol). The mixture was stirred at 25° C. for 1 hour. LC-MS showed Compound 7-3-3 was consumed completely and one main peak with desired MS was detected. The mixture was concentrated in vacuo. The residue was purified by column chromatography (SiO₂, Dichloromethane: Ethyl acetate=10/1 to 0:1 Dichloromethane: Ethyl acetate=100/1 to 0:1). Compound 7-3-3 (6.20 g, 40.58% yield) was obtained as a yellow solid. ¹H NMR (400 MHz, CDCl₃): δ=8.25 (s, 1H), 7.50-7.14 (m, 10H), 6.90-6.77 (m, 4H), 5.93 (d, J=2.0 Hz, 1H), 5.01 (br s, 1H), 4.53-4.44 (m, 1H), 4.06 (br d, J=6.8 Hz, 1H), 3.94 (dd, J=2.0, 5.1 Hz, 1H), 3.83-3.73 (m, 9H), 3.63 (s, 3H), 3.55-3.48 (m, 1H), 3.39 (dd, J=2.5, 11.1 Hz, 1H), 2.79 (q, J=7.1 Hz, 1H), 1.85-1.76 (m, 2H), 1.50-1.41 (m, 2H), 1.24 (br s, 22H), 0.87 (t, J=6.7 Hz, 3H). ¹³CNMR (100 MHz, CDCl₃): δ=172.37, 162.32, 158.66, 158.58, 158.55, 149.58, 144.63, 142.49, 135.55, 135.44, 130.14, 130.00, 129.94, 128.08, 127.86, 126.91, 113.51, 113.35, 99.62, 89.56, 87.56, 86.85, 83.77, 83.68, 74.14, 68.49, 61.77, 58.82, 55.24, 45.30, 36.10, 31.89, 29.84, 29.67, 29.63, 29.49, 29.37, 29.33, 25.42, 22.66, 14.79, 14.11, 9.74. LCMS: (M+H⁺): 850.4.

### Preparation of 5′-DMT-2′OMe-5-Lipid-3′—CNE Phosphoramidite

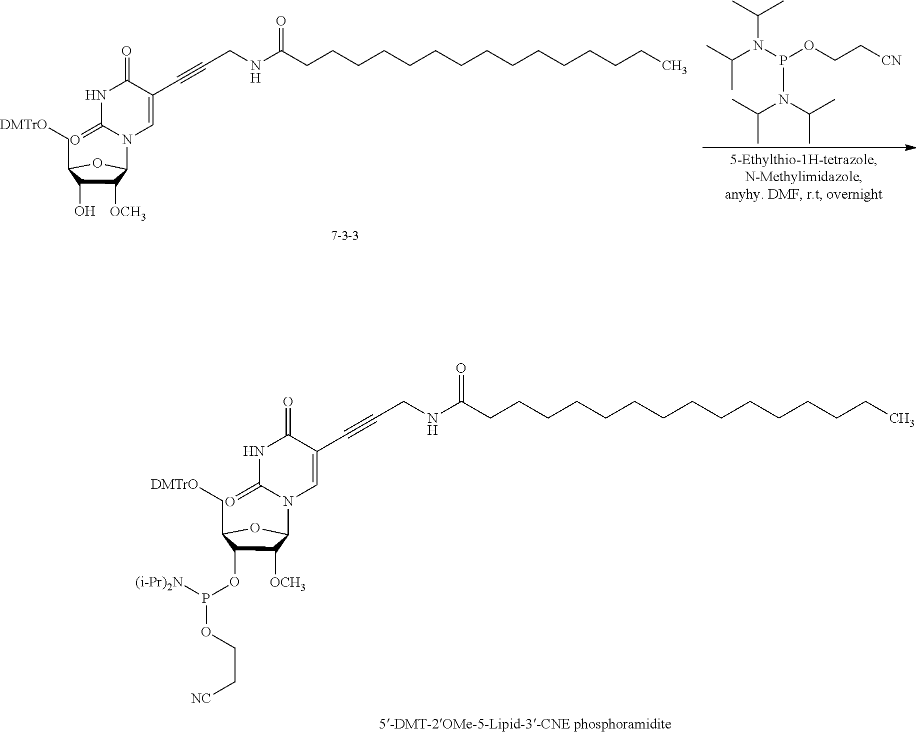

Compound 7-3-3 (2.8 g, 3.29 mmol) was co-evaporated with anhydrous toluene two times (25 mL×2) and dried under high vacuum overnight. The dried foamy solid was dissolved in anhydrous DMF (5 ml) and was added 5-ethylthio-1H-tetrazole (0.43 g, 3.29 mmol), N-methylimidazole (0.052 mL, 0.66 mmol) followed by 2-cynoethyl-N,N,N′,N′-tetraisopropylphosphordiamidite (1.49 g, 4.93 mmol). The reaction mixture was stirred at room temperature under argon atmosphere for overnight. After TLC indicated completion, the reaction was diluted with EtOAc (70 mL) and washed with aq. saturated. NaHCO₃ solution (10 mL), and dried over Mg₂SO₄. The solvent was evaporated under reduced pressure and dried in high vacuum for night. The dried crude product was purified by Combi-Flash Rf (Teledyne ISCO) using 80 g silica column (which was pre-deactivated with 3 column volume of ethyl acetate with 5% TEA) with Hexane/Ethyl acetate/Acetonitrile which contains 5% TEA as an eluent to afford 5′-DMT-2′OMe-5-Lipid-3′CNE phosphoramidite as a foamy solid. Yield 3.1 g (90%). ³¹P NMR (162 MHz, CDCl₃) δ 150.58(s) 150.26(s). ¹³C NMR (101 MHz, CDCl₃) δ 172.20, 172.18, 161.78, 161.66, 158.70, 158.68, 149.45, 149.35, 144.71, 144.57, 142.69, 142.62, 137.91, 135.63, 135.53, 135.49, 135.40, 130.16, 130.11, 128.08, 128.06, 128.01, 127.00, 126.97, 117.71, 117.51, 113.39, 113.36, 113.32, 99.75, 99.46, 89.30, 89.26, 88.49, 88.00, 87.05, 86.84, 83.86, 83.04, 82.98, 82.93, 82.66, 77.39, 77.27, 77.07, 76.75, 74.45, 74.30, 69.88, 69.77, 69.64, 62.10, 61.24, 58.94, 58.92, 58.65, 58.47, 58.44, 57.97, 57.76, 55.30, 55.27, 43.35, 43.32, 43.23, 43.19, 36.11, 36.09, 33.26, 31.90, 29.88, 29.67, 29.65, 29.63, 29.58, 29.50, 29.37, 29.33, 25.41, 24.70, 24.64, 24.61, 24.57, 24.54, 24.50, 22.66, 20.47, 20.40, 20.34, 20.27, 14.82, 14.09. ¹H NMR (400 MHz, Chloroform-d) δ 7.40 (dd, J=10.5, 7.6 Hz, 2H), 7.35-7.12 (m, 7H), 6.78 (ddd, J=9.0, 4.2, 2.7 Hz, 4H), 4.82 (dt, J=22.1, 4.9 Hz, 1H), 4.57-4.38 (m, 1H), 4.24-4.10 (m, 1H), 4.06-3.96 (m, 1H), 3.86-3.67 (m, 7H), 3.67-3.58 (m, 2H), 3.57-3.39 (m, 6H), 3.25 (ddd, J=13.5, 11.3, 2.8 Hz, 1H), 2.55 (t, J=6.1 Hz, 1H), 2.30 (t, J=6.2 Hz, 1H), 1.71 (qd, J=7.4, 7.0, 1.4 Hz, 2H), 1.38 (dtt, J=10.5, 7.7, 2.8 Hz, 2H), 1.09 (dd, J=6.7, 5.1 Hz, 17H), 0.97 (d, J=6.8 Hz, 3H), 0.80 (t, J=6.6 Hz, 3H). MS: LCMS: Calculated, C59H82N5O10P; 1051.5730; Observed +Ve mode: m/z: 1153.69 [M+Et₃N].

### Example 7-4. Synthesis of 5′-(R)—C-Me-5′-DMT-dT-CNE Amidite

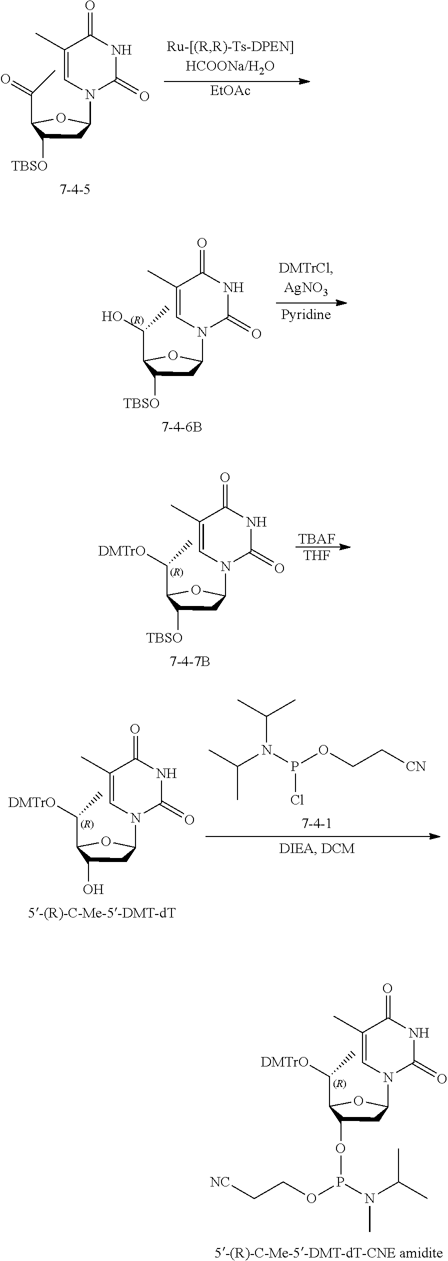

### Preparation of Compound 7-4-6B

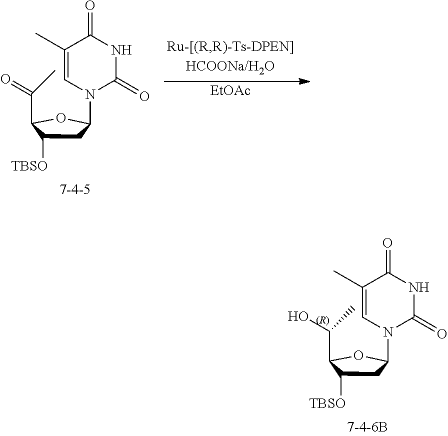

To a solution of compound 7-4-5 (46.00 g, 124.83 mmol) in a mixture of EtOAc (460.00 mL) and sodium formate (353.17 g, 5.19 mol) dissolved in Water (1.84 L), and then [[(1R,2R)-2-amino-1,2-diphenyl-ethyl]-(p-tolylsulfonyl)amino]-chloro-ruthenium; 1-isopropyl-4-methyl-benzene (1.59 g, 2.50 mmol) was added. The resulting two-phase mixture was stirred for 12 h at 25° C. under N2. TLC showed the starting material was consumed. The mixture was extracted with EtOAc (50 mL*3). The combined organic was washed with brine (30 mL), dried over Na₂SO₄, filtered and concentrated to get the crude. The residue was purified by re-crystallization from Petroleum ether/Ethyl acetate=5:1 to give the compound 7-4-6B as a white solid (36.00 g, 77.83% yield) as a white solid. ¹H NMR (400 MHz, DMSO-d₆): δ=11.31 (s, 1H), 7.67 (s, 1H), 6.16 (dd, J=5.5, 8.8 Hz, 1H), 5.05 (d, J=5.1 Hz, 1H), 4.49 (br d, J=5.1 Hz, 1H), 3.78-3.70 (m, 1H), 3.55 (d, J=3.7 Hz, 1H), 2.20-2.09 (m, 1H), 1.96 (br dd, J=5.7, 13.0 Hz, 1H), 1.77 (s, 3H), 1.11 (d, J=6.4 Hz, 3H), 0.87 (s, 9H), 0.09 (s, 6H). HPLC: HPLC purity: 97.7%. SFC: SFC purity: 99.1%. TLC (Petroleum ether/Ethyl acetate=1:1) Rf=0.37.

### Preparation of Compound 7-4-7B

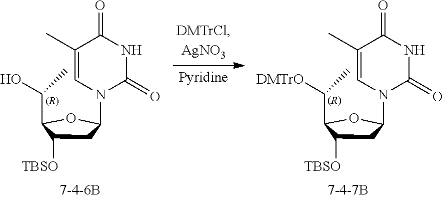

Compound 7-4-6B (18.00 g, 48.58 mmol) was dried by azeotropic distillation on a rotary evaporator with pyridine (100 mL) and toluene (100 mL*2). A solution of compound 7-4-6B (18.00 g, 48.58 mmol) and DMTCl (1.89 g, 5.59 mmol) in the mixture of pyridine (180.00 mL) and THF (720.00 mL) was degassed and purged with N₂ for 3 times and then AgNO₃ (14.19 g, 83.56 mmol) was added. The mixture was stirred at 25° C. for 15 hr. TLC showed the starting material was consumed. MeOH (5 mL) was added and stirred for 15 min and then the mixture was filtered and the cake was washed with toluene (300 mL*3). The filtrate was concentrated to obtain the compound 7-4-7B as a yellow oil (65.38 g, crude). The mixture was used directly to next step without any purification. TLC (Petroleum ether/Ethyl acetate) Rf=0.63.

### Preparation of 5′-(R)—C-Me-5′-DMTr-dT

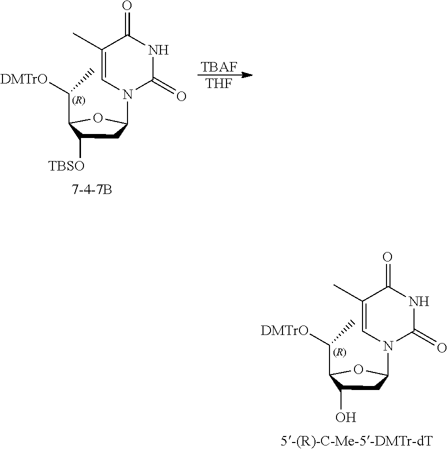

To a solution of compound 7-4-7B (65.38 g, 97.16 mmol) in THF (650.00 mL) was added TBAF (1 M, 184.60 mL). The mixture was stirred at 25° C. for 12 hours. TLC showed the starting material was consumed. The mixture was concentrated to provide the crude and then sat. NaCl (5% aq., 200 mL*2) was added and the mixture was extracted with EtOAc (200 mL*3). The combined organic phase was dried over Na₂SO₄, filtered and concentrated to provide the crude product, which was purified by MPLC (Petroleum ether:Ethyl acetate 5:1,1:1,1:4,5% TEA) to provide 5′-(R)—C-Me-5′-DMTr-dT as a white solid (47.50 g, 85.03 mmol, 87.52% yield). ¹H NMR (400 MHz, DMSO-d₆): δ=11.32 (s, 1H), 7.46 (br d, J=7.8 Hz, 2H), 7.37-7.25 (m, 6H), 7.23-7.16 (m, 1H), 7.07 (s, 1H), 6.89 (dd, J=4.6, 8.5 Hz, 4H), 6.12 (t, J=7.2 Hz, 1H), 5.27 (d, J=4.6 Hz, 1H), 4.54-4.46 (m, 1H), 3.73 (d, J=1.8 Hz, 6H), 3.62 (t, J=2.9 Hz, 1H), 3.40-3.34 (m, 1H), 2.09-2.02 (m, 2H), 1.40 (s, 3H), 0.77 (d, J=6.2 Hz, 3H). ¹³C NMR (101 MHz, DMSO-d₆): δ=163.98, 158.58, 150.81, 146.95, 137.11, 136.79, 135.76, 130.49, 130.41, 128.20, 128.15, 127.04, 113.54, 113.52, 110.16, 89.87, 86.24, 83.35, 70.28, 70.05, 60.20, 55.47, 55.35, 21.20, 17.82, 14.52, 12.08. HPLC: HPLC purity: 98.7%. LC-MS: (M−H⁺)=557.2. LCMS purity: 98.9%. SFC: SFC purity: 100.0%. TLC (Petroleum ether/Ethyl acetate=1:1, 5% TEA) Rf=0.02.

### Preparation of 5′-(R)—C-Me-5′-DMT-dT-CNE-Amidite

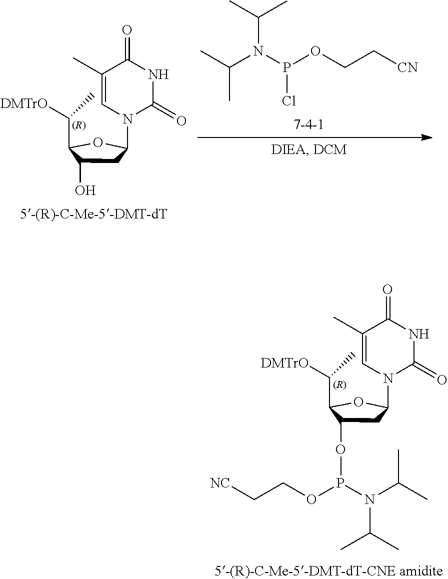

5′-(R)—C-Me-5′-OMT-dT (5 g, 8.95 mmol) was dried with toluene (50 mL). To a solution of DIEA (1.39 g, 10.74 mmol, 1.87 mL) and 5′-(R)—C-Me-5′-DMT-dT (5 g, 8.95 mmol) in anhydrous DCM (50 mL) was added compound 7-4-1 (2.76 g, 9.40 mmol) under N2 at 0° C. The mixture was stirred at 15° C. for 2 h. TLC showed the starting material was consumed and two new spots were found. The mixture was quenched by addition of saturated aq. NaHCO₃(20 mL) and extracted with DCM (30 mL*3). The combined organic phase was dried over Na₂SO₄, filtered and concentrated to provide the crude product, which was purified on a Combiflash instrument from Teledyne. A 40 g silica gel cartridge column was first pre-treated by eluting with 10% EtOAc/Petroleum ether containing 5% Et₃N (300 mL). The crude product was dissolved in a 2:1 volume:volume mixture of methylene chloride:petroleum ether containing 5% Et₃N and loaded onto the column. After loading, the purification process was run using the following gradient: 10 to 50% EtOAc/Petroleum ether containing 5% Et₃N. Fractions were collected. After evaporation of the solvent, 5′-(R)—C-Me-5′-DMT-dT-CNE-amidite was obtained as a white solid (3.6 g, 53% yield). ¹H NMR (400 MHz, CHLOROFORM-d) δ=8.11 (br s, 1H), 7.53 (br d, J=7.7 Hz, 3H), 7.42 (br t, J=8.2 Hz, 4H), 7.32-7.17 (m, 4H), 7.07-6.99 (m, 1H), 6.84 (br d, J=8.2 Hz, 4H), 6.31 (br dd, J=5.5, 8.7 Hz, 1H), 4.94 (br s, 1H), 3.96-3.73 (m, 10H), 3.72-3.41 (m, 4H), 2.65 (td, J=6.1, 18.0 Hz, 2H), 2.53-2.37 (m, 1H), 2.10 (br d, J=8.2 Hz, 1H), 1.47 (br s, 4H), 1.33-1.16 (m, 15H), 1.00-0.90 (m, 3H). ³¹P NMR (162 MHz, CHLOROFORM-d) δ=148.81 (s, 1P), 148.35 (s, 1P).

### Example 7-5. Synthesis of 5′-(S)—C-Me-5′-DMT-dT-CNE Amidite

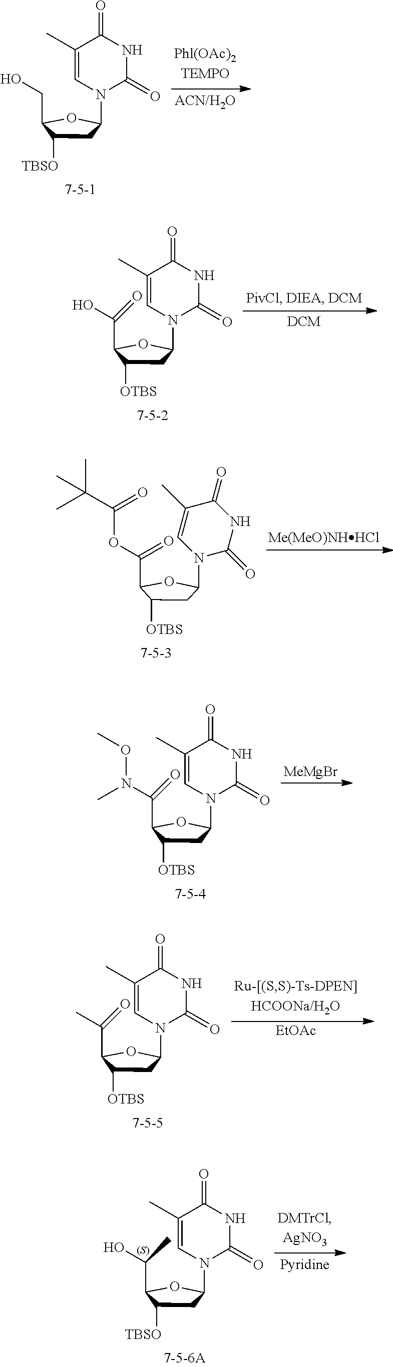

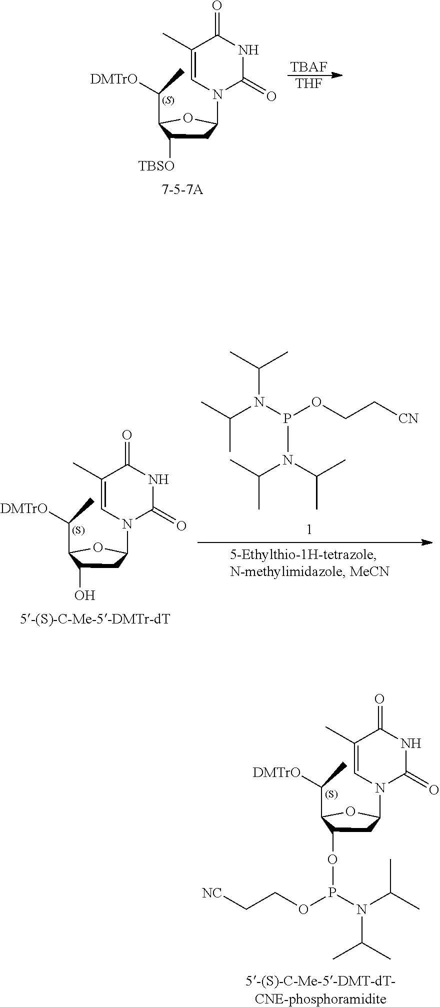

### Preparation of Compound 7-5-2

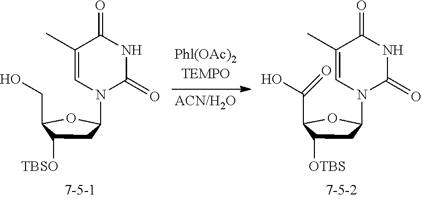 [SMILES: Cc1cn([C@H]2C[C@@H](O[Si](C)(C)C(C)(C)C)[C@@H](CO)O2)c(=O)[nH]c1=O ; Cc1cn([C@H]2C[C@@H](O[Si](C)(C)C(C)(C)C)[C@@H](C(=O)O)O2)c(=O)[nH]c1=O]

To a solution of compound 7-5-1 (63.00 g, 176.72 mmol) in the mixture of H₂O (250.00 mL) and MeCN (250.00 mL) was added PhI(OAc)₂ (125.23 g, 388.79 mmol) and TEMPO (5.56 g, 35.34 mmol) at 10° C. The mixture was stirred at 25° C. for 2 hours. TLC (Petroleum ether/Ethyl acetate=1:1, Rf=0) showed the starting material was consumed. The mixture was concentrated, and MTBE (1 L) was added. The mixture was stirred for 0.5 h and then filtered. The cake was washed with MTBE (1 L*2), and dried to provide compound 7-5-2 as a white solid (126 g, 96.23% yield). ¹H NMR (400 MHz, DMSO): δ=11.21 (s, 1H), 7.89 (d, J=1.0 Hz, 1H), 6.18 (dd, J=5.9, 8.6 Hz, 1H), 4.61-4.41 (m, 1H), 4.17 (d, J=0.9 Hz, 1H), 2.51-2.26 (m, 3H), 2.09-1.85 (m, 2H), 1.74-1.58 (m, 3H), 0.90-0.58 (m, 10H), 0.00 (d, J=2.0 Hz, 6H). LC-MS: (M+H⁺): 371.1. TLC (Petroleum ether/Ethyl acetate=1:1) Rf=0.

### Preparation of Compound 7-5-3

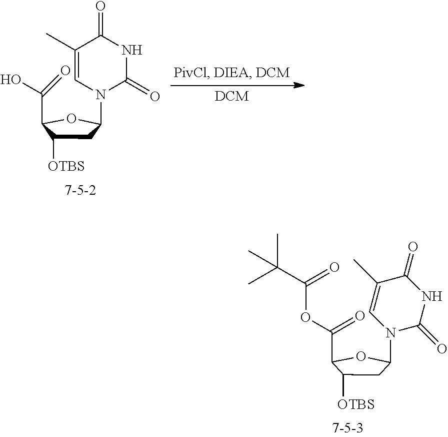 [SMILES: Cc1cn([C@H]2C[C@@H](O[Si](C)(C)C(C)(C)C)[C@@H](C(=O)O)O2)c(=O)[nH]c1=O ; Cc1cn(C2CC(O[Si](C)(C)C(C)(C)C)C(C(=O)OC(=O)C(C)(C)C)O2)c(=O)[nH]c1=O]

To a solution of compound 7-5-2 (50.00 g, 134.96 mmol) in DCM (500.00 mL) was added DIEA (34.89 g, 269.92 mmol, 47.15 mL) and 2,2-dimethylpropanoyl chloride (21.16 g, 175.45 mmol). The mixture was stirred at −10-0° C. for 1.5 hours. TLC showed the starting material was consumed. The mixture in DCM was used directly for next step. TLC (Petroleum ether/Ethyl acetate=1:1) Rf=0.15.

### Preparation of Compound 7-5-4

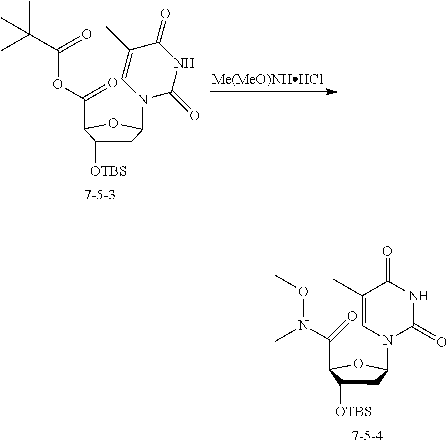

To compound 7-5-3 in DCM was added TEA (40.94 g, 404.55 mmol, 56.08 mL) and N-methoxymethanamine hydrochloride (19.73 g, 202.27 mmol). The mixture was stirred at 0° C. for 1 h. TLC showed the starting material was consumed. The mixture was washed with HCl (1N, 100 mL) and then aqueous NaHCO₃(100 mL). The organic layer was dried over Na₂SO₄, filtered and concentrated to provide the crude product, which was purified by silica gel chromatography (Petroleum ether/Ethyl acetate=30/1, 0/1) to afford compound 7-5-4 as a white solid (95.5 g, 85.63% yield). ¹H NMR (400 MHz, CDCl₃): δ=8.29 (s, 1H), 8.19 (br s, 1H), 6.46 (dd, J=5.1, 9.3 Hz, 1H), 4.71 (s, 1H), 4.38 (d, J=4.2 Hz, 1H), 3.65 (s, 3H), 3.15 (s, 3H), 2.18-2.08 (m, 1H), 2.00-1.90 (m, 1H), 1.87 (d, J=1.1 Hz, 3H), 0.88-0.74 (m, 10H), 0.00 (d, J=3.7 Hz, 6H). TLC (Petroleum ether/Ethyl acetate=1:1) Rf=0.43.

### Preparation of Compound 7-5-5

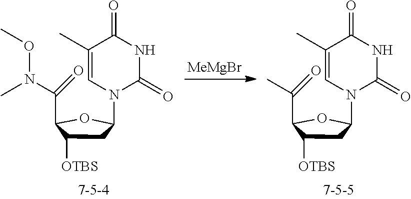 [SMILES: "MeMgBr" ; CON(C)C(=O)[C@H]1O[C@@H](n2cc(C)c(=O)[nH]c2=O)C[C@H]1O[Si](C)(C)C(C)(C)C ; CC(=O)[C@H]1O[C@@H](n2cc(C)c(=O)[nH]c2=O)C[C@H]1O[Si](C)(C)C(C)(C)C]

To a solution of compound 7-5-4 (115.00 g, 278.09 mmol) in THF (1.20 L) was added MeMgBr (3 M, 185.39 mL) at 0° C. The mixture was stirred at 0° C. for 2 h. TLC showed the starting material was consumed. To the mixture was added water (1 L) at 0° C. and the mixture was extracted with EtOAc (300 mL*2). The combined organic phase was dried over Na₂SO₄, filtered and concentrated to provide the compound 7-5-5 as a white solid (100.00 g, 97.58% yield). The mixture was used directly without further purification. ¹H NMR (400 MHz, CDCl₃): δ=8.81 (br s, 1H), 7.95 (s, 1H), 6.41 (dd, J=5.6, 8.1 Hz, 1H), 4.60-4.40 (m, 2H), 2.40-2.16 (m, 4H), 1.98 (s, 3H), 1.02-0.83 (m, 10H), 0.14 (d, J=3.3 Hz, 6H), 0.20-0.00 (m, 1H). TLC (Petroleum ether/Ethyl acetate=1:1) Rf=0.68.

### Preparation of Compound 7-5-6A

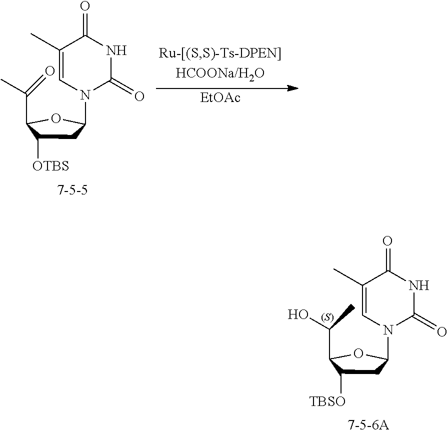

To a solution of compound 7-5-5 (46.00 g, 124.83 mmol) in the mixture of EtOAc (460.00 mL) and sodium formate (353.17 g, 5.19 mol) dissolved in water (1.84 L), and N-[(1S,2S)-2-amino-1,2-diphenyl-ethyl]-4-methyl-benzenesulfonamide chlororuthenium; 1-isopropyl-4-methyl-benzene (1.59 g, 2.50 mmol) was added. The resulting two-phase mixture was stirred for 12 h at 25° C. under N2. TLC showed the starting material was consumed. The mixture was extracted with EtOAc (500 mL*3). The combined organic was washed with brine (300 mL), dried over Na₂SO₄, filtered and concentrated to provide the crude product. The mixture was purified by MPLC (Petroleum ether/MTBE=10:1 to 1:1) seven times to provide compound 7-5-6A as a yellow oil (25.6 g, 57.53% yield). ¹H NMR (400 MHz, DMSO-d₆): δ=11.28 (s, 1H), 7.85 (s, 1H), 6.16 (t, J=6.8 Hz, 1H), 5.04 (d, J=4.6 Hz, 1H), 4.46-4.29 (m, 1H), 3.79 (br t, J=6.8 Hz, 1H), 3.59 (br s, 1H), 3.32 (s, 1H), 2.21-2.09 (m, 1H), 2.06-1.97 (m, 1H), 1.76 (s, 3H), 1.17-1.08 (m, 4H), 0.91-0.81 (m, 10H), 0.08 (s, 6H). SFC: SFC purity: 98.6%. TLC (Petroleum ether/Ethyl acetate=1:1) Rf=0.38.

### Preparation of Compound 7-5-7A

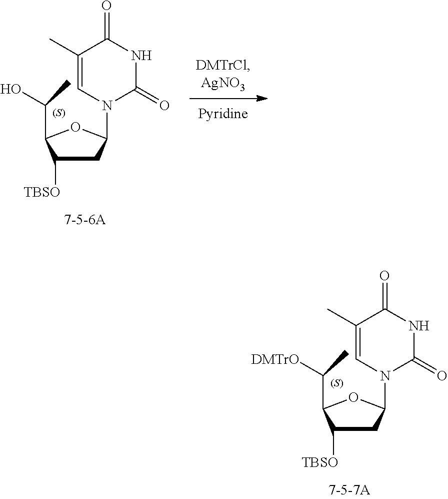

Compound 7-5-6A (12.80 g, 34.55 mmol) was dried by azeotropic distillation on a rotary evaporator with pyridine (100 mL) and toluene (100 mL*2). To a solution of compound 7-5-6A (12.80 g, 34.55 mmol) and DMTCl (1.89 g, 5.59 mmol) in the mixture of pyridine (120.00 mL) and THF (400.00 mL) was degassed and purged with N₂ for 3 times and then AgNO₃ (10.09 g, 59.43 mmol) was added. The mixture was stirred at 25° C. for 15 hr. TLC showed the starting material was consumed. MeOH (5 mL) was added and stirred for 15 min and then the mixture was filtered and the cake was washed with toluene (300 mL*3). The filtrate was concentrated to get the compound 7-7-7A as a yellow oil (46.50 g, crude). The mixture was used directly to next step without any purification. TLC (Petroleum ether/Ethyl acetate) Rf=0.63.

### Preparation of 5′-(S)—C-Me-5′-DMT-dT

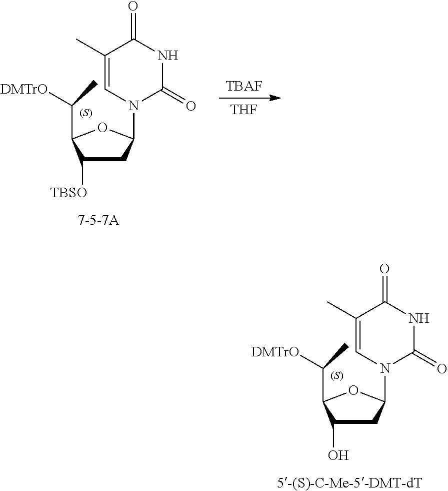

To a solution of compound 7-5-7A (46.50 g, 69.11 mmol) in THF (460.00 mL) was added TBAF (1 M, 131.31 mL). The mixture was stirred at 25° C. for 5 hrs. TLC showed the starting material was consumed. The mixture was concentrated and then sat. NaCl (5% aq., 200 mL) was added and the aqueous phase was extracted with EtOAc (200 mL*3). The combined organic layer was dried over Na₂SO₄, filtered and concentrated to provide the crude product, which was purified by MPLC (Petroleum ether/Ethyl acetate 5:1, 1:1, 1:4, 5% TEA) to provide 5′-(S)—C-Me-5′-DMT-dT as a white solid (29.0 g, 75.12% yield). ¹H NMR (400 MHz, DMSO-d₆): δ=11.35 (s, 1H), 7.56 (s, 1H), 7.58-7.53 (m, 1H), 7.44 (d, J=7.8 Hz, 2H), 7.37-7.24 (m, 6H), 7.23-7.17 (m, 1H), 6.87 (t, J=8.3 Hz, 4H), 6.13 (t, J=6.9 Hz, 1H), 5.21 (d, J=4.9 Hz, 1H), 4.23 (br s, 1H), 3.73 (d, J=2.9 Hz, 6H), 3.67 (t, J=3.7 Hz, 1H), 3.57-3.46 (m, 1H), 2.23-2.04 (m, 2H), 1.67 (s, 3H), 1.70-1.65 (m, 1H), 0.71 (d, J=6.2 Hz, 3H). ¹³CNMR (101 MHz, DMSO-d₆): δ=170.78, 164.16, 158.64, 158.59, 150.86, 146.71, 137.00, 136.75, 135.97, 130.65, 130.52, 128.38, 128.07, 127.11, 113.48, 110.11, 89.78, 86.41, 83.87, 70.58, 70.22, 60.21, 55.48, 21.20, 18.08, 14.53, 12.54. HPLC: HPLC purity: 98.4%. LCMS: (M−H+)=557.2; LCMS purity: 99.0%. SFC: SFC purity: 99.4%. TLC (Petroleum ether/Ethyl acetate=1:1, 5% TEA) Rf=0.01.

### Preparation of 5′-(S)—C-Me-5′-DMT-dT-CNE-Amidite

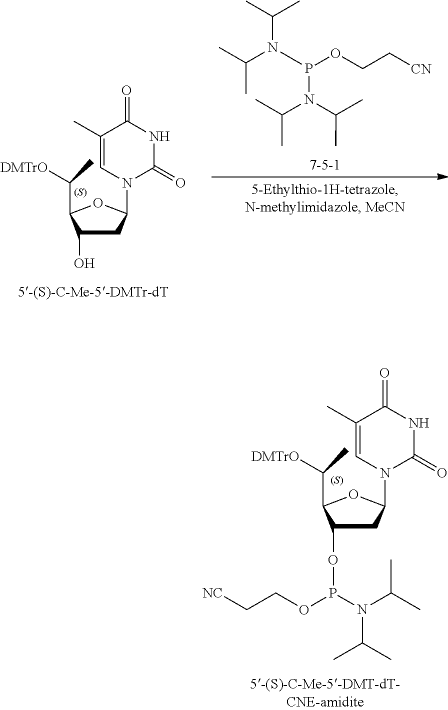

To a solution of 5′-(S)—C-Me-5′-DMT-dT (5.00 g, 8.95 mmol) in MeCN (50.00 mL) was added 5-ethylsulfanyl-2H-tetrazole (1.17 g, 8.95 mmol), 1-methylimidazole (1.47 g, 17.90 mmol, 1.43 mL) and compound 7-5-1 (4.05 g, 13.43 mmol, 4.26 mL). The reaction mixture was stirred at 20° C. under N2 for 2 hrs. TLC and LC-MS showed some starting material was consumed and the desired substance was formed. The reaction mixture was concentrated under reduced pressure, and the residue was diluted with EtOAc (20 mL). The reaction mixture was washed with aq. saturated NaHCO₃ solution (20 mL), dried over Na₂SO₄, filtered and concentrated to provide the crude product, which was purified by MPLC (Petroleum ether 5% TEA: Ethyl acetate from 10:1 to 1:1) to provide 5′-(S)—C-Me-5′-DMT-dT-CNE-amidite as a white solid (4.3 g, 63.31% yield). ¹H NMR (400 MHz, CHLOROFORM-d) δ=8.19 (br s, 1H), 7.69-7.60 (m, 1H), 7.54 (s, 1H), 7.43-7.33 (m, 2H), 7.32-7.07 (m, 8H), 6.73 (ddd, J=3.7, 5.8, 9.0 Hz, 4H), 6.27-6.15 (m, 1H), 4.49-4.37 (m, 1H), 3.82-3.65 (m, 8H), 3.63-3.55 (m, 2H), 3.53-3.39 (m, 3H), 2.50 (t, J=6.3 Hz, 1H), 2.46-2.31 (m, 1H), 2.29-2.19 (m, 1H), 2.16-2.04 (m, 1H), 1.68 (s, 3H), 1.20-1.00 (m, 13H), 0.95 (d, J=6.8 Hz, 3H), 0.92-0.74 (m, 4H). ³¹P NMR (162 MHz, CHLOROFORM-d) δ=149.11 (s, 1P), 148.99 (s, 1P).

### Example 7-6. Synthesis of L-DPSE-5′-(R)—C-Me-5′-DMT-dT Amidite

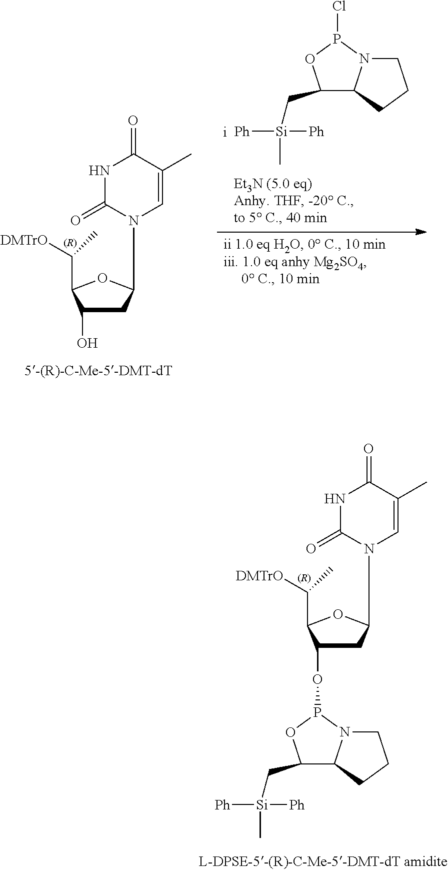

The 5′-(R)—C-Me-5′-OMT-dT (11.17 g, 20 mmol) was dried two times by co-evaporation with 80 mL of anhydrous toluene at 45° C. and kept at high vacuum for overnight. Then the dried 5′-(R)—C-Me-5′-OMT-dT was dissolved in dry THF (80 mL) in 500 mL three neck flasks under argon, followed by the addition of triethylamine (13.93 mL, 100 mmol) and the mixture was cooled to −40° C. To this cooled reaction mixture was added the solution of the crude L-DPSE-NOPCl (30 mmol, 1.4 eq, in THF 40 mL), from a stock through syringe dropwise (˜15 min, maintaining the internal temperature −40- to −35° C.). The mixture was then gradually warmed to 5° C. After 30 min at 5° C., TLC and LC-MS analysis indicated the complete conversion of SM to product (total reaction time 2 h). The reaction mixture was cooled in an ice bath and the reaction quenched by addition of water (0.36 mL, 20 mmol). The mixture was stirred for 10 min followed by addition of anhydrous Mg₂SO₄ (3.0 g, 20 mmol). The reaction was filtered through Airfree, Schlenk filter tube, washed with dry THF (60 mL) and the solvent was evaporated under rotary evaporation at 28° C. to afford the crude product as a off-white solid which was dried under high vacuum for overnight. The dried crude product was purified by Combi-Flash Rf (Teledyne ISCO) using a 220 silica column (which was pre-deactivated with 3 column volume of ethyl acetate with 5% TEA) with ethyl acetate/hexane mixture contains 5% TEA as a solvent. Fractions were analyzed by TLC and LC-MS and pooled together. Solvent was evaporated in a rotary evaporator at 28° C. and the residue was dried under high vacuum to afford the product as a white solid. Yield: 16.3 g (91%). ¹H NMR (400 MHz, Chloroform-d) δ 7.50-7.36 (m, 6H), 7.35-7.06 (m, 13H), 6.85 (d, J=1.4 Hz, 1H), 6.73 (dq, J=8.7, 3.2 Hz, 4H), 6.13 (dd, J=9.3, 5.3 Hz, 1H), 5.10 (td, J=7.8, 7.1, 3.4 Hz, 1H), 4.80 (dt, J=8.6, 5.8 Hz, 1H), 4.04 (q, J=7.1 Hz, 1H), 3.69 (d, J=2.3 Hz, 6H), 3.57-3.36 (m, 3H), 3.29-3.05 (m, 2H), 2.05 (dd, J=13.6, 5.5 Hz, 1H), 1.96 (s, 2H), 1.73-1.50 (m, 3H), 1.47-1.32 (m, 2H), 1.30 (d, J=1.2 Hz, 3H), 1.17 (t, J=7.2 Hz, 2H), 0.75 (d, J=6.5 Hz, 3H), 0.60 (s, 3H). ³¹P NMR (162 MHz, CDCl₃) δ 151.34 (s). MS: LCMS: Calculated, C51H56N3O8 PSi, 897.3574; Observed +Ve mode: m/z: 898.52 [M+H]; 999.95 [M+Et₃N]. ¹³C NMR (101 MHz, CDCl₃) δ 171.12, 163.83, 158.65, 158.61, 150.21, 146.50, 136.96, 136.71, 136.59, 135.94, 135.54, 134.60, 134.34, 130.24, 130.15, 129.45, 129.39, 128.02, 127.96, 127.94, 127.88, 127.79, 126.86, 113.17, 113.11, 110.93, 89.27, 89.25, 86.48, 83.68, 79.09, 78.99, 77.42, 77.30, 77.10, 76.78, 71.78, 71.70, 70.26, 68.39, 68.36, 60.39, 55.24, 47.19, 46.83, 46.09, 39.48, 39.44, 27.35, 25.97, 25.93, 21.05, 18.33, 17.85, 17.81, 14.23, 11.73, 11.45.

### Example 7-7. Synthesis of L-DPSE-5′-(S)—C-Me-5′-DMT-dT Amidite

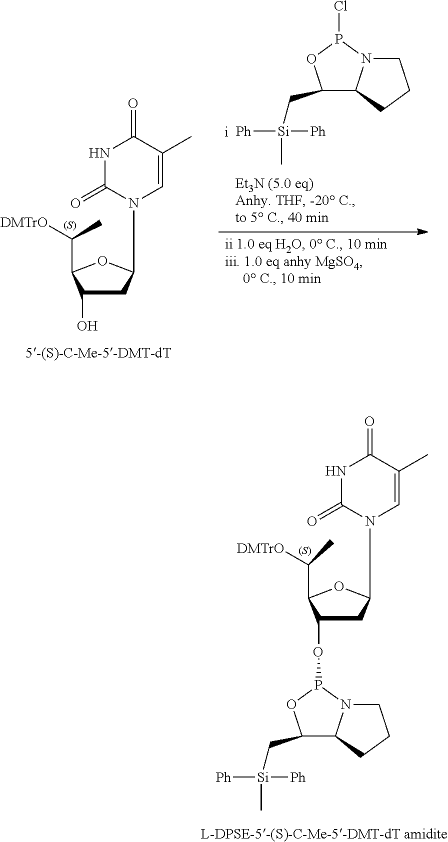

5′-(S)—C-Me-5′-OMT-dT (1.20 g, 2 mmol) was dried two times by co-evaporation with 20 mL of anhydrous toluene at 45° C. and kept at high vacuum for overnight. Then the dried 5′-(S)—C-Me-5′-OMT-dT was dissolved in dry THF (20 mL) in a 100 mL three neck flasks under argon, followed by the addition of triethylamine (1.4 mL, 10 mmol) and the mixture was cooled to −40° C. To this cooled reaction mixture was added the solution of the crude L-DPSE-NOPCl (3 mmol, 1.5 eq, in THF 3.0 mL) from a stock was through syringe dropwise ˜5 min (maintaining the internal temperature −40° C., then gradually warmed to 5° C.). After 30 min at 5° C., TLC and LC-MS analysis indicated complete conversion of SM to product (total reaction time 1.5 h). The reaction mixture was cooled in an ice bath and the reaction was quenched by addition of water (0.036 mL, 2 mmol). The mixture was stirred for 10 min, followed by addition of anhydrous MgSO₄ (0.3 g, 2 mmol). The reaction was filtered through Airfree, Schlenk filter tube and washed with dry THF (20 mL). The solvent was evaporated under rotary evaporation at 28° C. to provide the off-white solid which was dried under high vacuum for overnight. The dried crude product was purified by Combi-Flash Rf (Teledyne ISCO) using 40 g silica column (which was pre-deactivated with 3 column volume of ethyl acetate with 5% TEA) with ethyl acetate/hexane mixture containing 5% TEA as a solvent. After column purification, fractions were analyzed by TLC and LC-MS and were pooled together and evaporated in a rotary evaporator at 28° C. The residue was dried under high vacuum to afford L-DPSE-5′-(S)—C-Me-5′-DMT-dT amidite as a white solid. Yield: 1.27 g (70%). ³¹P NMR (162 MHz, CDCl₃) δ 149.73 (s). MS: LC-MS; Calculated: C₅₁H56N3O8 PSi, 897.3574; Observed +Ve mode: m/z: 898.56[M+H].

### Example 7-8. Synthesis of L-DPSE-5′-DMT-5-C₆-aminolinker Amidite—Incorporation of Desired Moieties Through Nucleobases

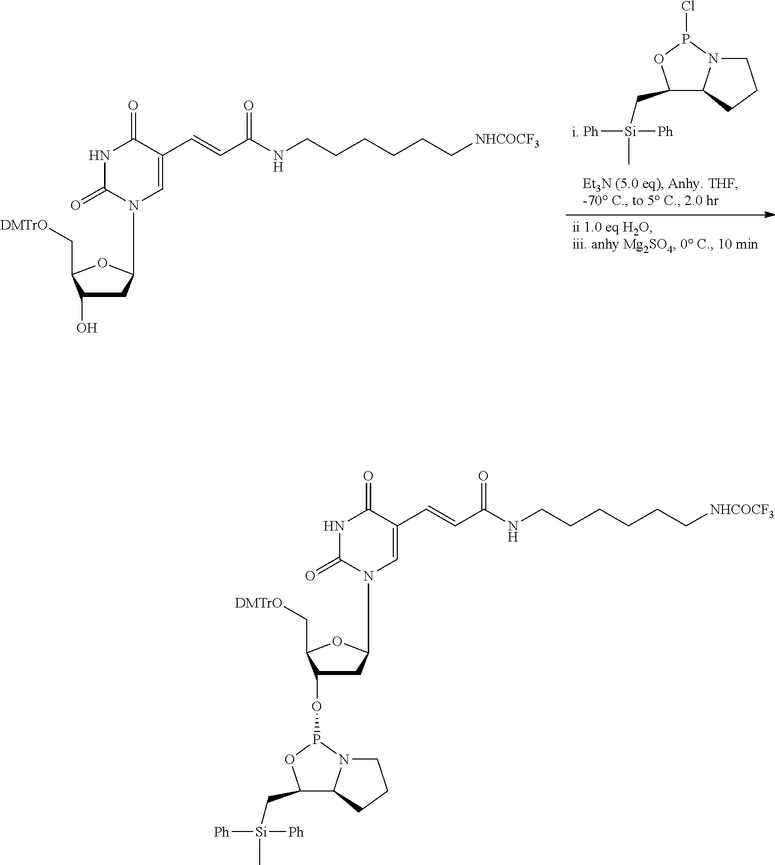

The 5′-DMT-5-C₆ aminoTFA-dT (25 g, 31.5 mmol, from Berry& Associates Inc) was dried two times by co-evaporation with 100 mL of anhydrous toluene at 45° C. and kept at high vacuum for overnight. Then the dried material was dissolved in dry THF (100 mL) in 500 mL three neck flasks under argon, followed by the addition of triethylamine (21.92 mL, 157 mmol) and then was cooled to −70° C. To this cooled reaction mixture was added a solution of the crude L-DPSE-NOPCl (44 mmol, 1.4 eq, in THF 44 mL), from a stock via syringe dropwise (˜15 min, maintaining the internal temperature −60- to 50° C.). The mixture was gradually warmed to 5° C. After 30 min at 5° C., TLC and LC-MS analysis indicated complete conversion of SM to product (total reaction time 2 h). The reaction mixture was cooled in an ice bath and quenched by addition of water (0.56 mL, 31.5 mmol), and stirred for 10 min followed by added anhydrous Mg₂SO₄ (3.8 g, 31.5 mmol). The reaction was filtered through Airfree, Schlenk filter tube, washed with dry THF (80 mL), and evaporated under rotary evaporation at 28° C. to afford the crude product as off-white solid, which was dried under high vacuum for overnight. The dried crude product was purified by Combi-Flash Rf (Teledyne ISCO) using 220 silica column (which was pre-deactivated with 3 column volume of ethyl acetate with 5% TEA) with ethyl acetate/hexane mixture contains 5% TEA as a solvent. After column purification fractions were analyzed by TLC and LC-MS, and pooled together. Solvent was evaporated in a rotary evaporator at 28° C. and the residue was dried under high vacuum to afford the product as a white solid. Yield: 30 g (88%). MS: LC-MS; Calculated: C60H67F3N5O10 PSi, 1133.4347; Observed in +Ve mode: 1235.55 (M+Et₃N). ¹H NMR (400 MHz, Chloroform-d) δ 7.78 (s, 1H), 7.40 (ddd, J=9.8, 6.5, 2.2 Hz, 5H), 7.32 (d, J=7.3 Hz, 2H), 7.30-7.09 (m, 15H), 6.99 (d, J=15.5 Hz, 1H), 6.76 (dd, J=8.9, 2.7 Hz, 4H), 6.54 (d, J=15.5 Hz, 1H), 5.12 (t, J=6.1 Hz, 1H), 4.66-4.49 (m, 2H), 4.04 (q, J=7.1 Hz, 1H), 3.81 (q, J=3.0 Hz, 1H), 3.67 (s, 6H), 3.41 (ddt, J=14.8, 10.2, 7.7 Hz, 1H), 3.30-3.13 (m, 4H), 3.12-2.91 (m, 4H), 1.96 (s, 2H), 1.92-1.69 (m, 2H), 1.58 (ddt, J=15.1, 11.6, 8.0 Hz, 1H), 1.50-1.29 (m, 5H), 1.18 (tq, J=15.8, 8.8, 8.0 Hz, 9H), 0.52 (s, 3H). ³¹P NMR (162 MHz, CDCl₃) δ 150.88 (s). ¹³C NMR (101 MHz, CDCl₃) δ 171.18, 165.77, 161.89, 158.76, 158.74, 157.85, 157.49, 157.12, 156.76, 149.17, 144.52, 139.69, 136.68, 135.86, 135.53, 135.44, 134.54, 134.30, 131.15, 129.97, 129.89, 129.44, 129.38, 128.09, 127.93, 127.91, 127.18, 122.36, 120.31, 117.44, 114.58, 113.42, 113.39, 111.72, 110.53, 86.65, 86.04, 86.02, 85.67, 79.28, 79.19, 77.42, 77.31, 77.11, 76.79, 73.20, 73.12, 68.05, 68.02, 63.09, 60.41, 55.27, 46.96, 46.60, 45.81, 40.48, 39.56, 38.88, 29.33, 28.52, 27.23, 25.83, 21.04, 17.55, 17.52, 14.20.

### Example 7-9. Synthesis of 5-Alkynyl thioacetate-5′-DMT-3′CNE-2′OMe-U Amidite

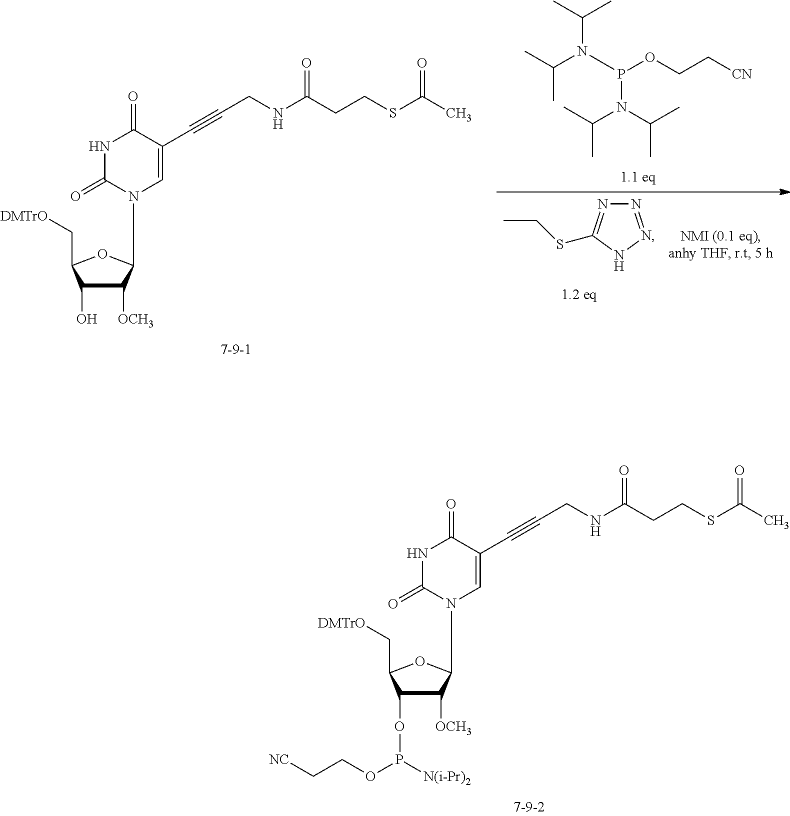

Compound 7-9-1 (5.0 g, 6.72 mmol) was co-evaporated with anhydrous toluene two times (40 mL×2) and dried under high vacuum for overnight. The dried yellow solid was dissolved in anhydrous THF (14 ml,-0.5 mmol/mL) under argon and to the solution was added 5-ethylthio-1H-tetrazole (1.05 g, 8.07 mmol), N-methylimidazole (0.045 g, 0.044 mL, 0.67 mmol) followed by 2-cynoethyl-N,N,N′,N′-tetraisopropylphosphordiamidite (2.23 g, 2.34 mL, 7.39 mmol). The reaction mixture was stirred at room temperature under argon for 5 h. TLC (solvent system: 40% CH₃CN/EtOAC/5% TEA) which was pre-equilibrated with the above solvent system indicated the completion of reaction at 5 h, which was also confirmed with LC-MS. The reaction mixture was diluted with EtOAc (100 mL) and the solution was transferred to separating funnel, washed with aq. saturated. NaHCO₃ solution (40 mL) and dried over anhydrous Mg₂SO₄. The dried solution was evaporated under rotary evaporation at bath temperature 28° C. to afford the crude product as off-yellow solid which was further dried under high vacuum for overnight. The dried crude product was purified in Combi-Flash Rf (Teledyne ISCO) using 80 g flash silica column, which was pre-deactivated with 2 column volume (CV 125 mL, 60 mL/min), of ethyl acetate with 5% TEA, followed by equilibration with 20% EtOAc/Hexane for 2 column volume. The compound was purified using Hexane/EtOAc/CH₃CN mixture containing 5% TEA as a solvent system. After purification column fractions were analyzed by TLC and LC-MS. Desired fractions were pooled together and evaporated in a rotary evaporator at 28° C. and was dried under high vacuum afforded 7-9-2-CNE amidite as white solid. Yield: 4.8 g (76%). MS: LC-MS; Calculated: C48H58N5O11PS, 943.35; Observed in +Ve mode: m/z 1045.92 (M+Et₃N). ¹H NMR (400 MHz, Chloroform-d) δ 8.36-8.07 (m, 1H), 7.47-7.09 (m, 10H), 6.78 (dt, J=9.1, 3.8 Hz, 4H), 5.87 (dd, J=26.6, 3.1 Hz, 1H), 4.73 (d, J=14.9 Hz, 1H), 4.57-4.30 (m, 1H), 4.21-4.00 (m, 2H), 3.86-3.32 (m, 17H), 3.23 (ddd, J=13.0, 11.2, 2.5 Hz, 1H), 2.91 (td, J=7.0, 2.4 Hz, 2H), 2.54 (q, J=6.1 Hz, 1H), 2.27 (d, J=24.2 Hz, 4H), 1.96 (d, J=7.1 Hz, 3H), 1.21-0.82 (m, 14H). ³¹P NMR (162 MHz, CDCl₃) δ 150.60 (s), 150.24(s). ¹³C NMR (101 MHz, CDCl₃) δ 195.80, 169.61, 161.45, 158.70, 158.68, 149.06, 144.75, 144.61, 142.82, 135.67, 135.58, 135.48, 135.38, 130.18, 130.16, 130.12, 128.14, 128.11, 128.09, 128.02, 127.01, 117.69, 117.53, 113.42, 113.38, 113.34, 99.60, 99.33, 88.98, 88.95, 88.50, 88.06, 87.06, 86.85, 83.89, 82.99, 82.62, 77.34, 77.22, 77.02, 76.70, 74.55, 74.40, 69.74, 69.62, 62.04, 61.26, 60.38, 58.97, 58.59, 58.47, 58.45, 57.89, 57.68, 55.34, 55.31, 43.33, 43.21, 35.44, 35.41, 30.54, 29.95, 24.71, 24.65, 24.63, 24.58, 24.56, 24.49, 21.04, 20.50, 20.43, 20.38, 20.31, 14.20.

As readily appreciated by those skilled in the art, compound 7-9-2 can be utilized in oligonucleotide synthesis as a phosphoramidite in accordance with the present disclosure, thereby incorporating a protected thiol group into oligonucleotides. After deprotection, free thiol groups can be utilized to link oligonucleotide monomers to form multimers, by forming one or more disulfide bonds, in accordance with the present disclosure.

As appreciated by a person having ordinary skill in the art, many technologies (e.g., chemistry, reagents, linkers, methods, etc.) can be utilized to prepare oligonucleotides (including those with various 5′-end structures) and to incorporate various chemical moieties, e.g., carbohydrate moieties, lipid moieties, targeting moieties, etc., into oligonucleotides in accordance with the present disclosure, for example but not limited to those described in WO/2010/064146, WO/2011/005761, WO/2013/012758, WO/2014/010250, US2013/0178612, WO/2014/012081, WO/2015/107425, WO/2017/015555, and WO/2017/062862. Described herein are example technologies for preparing oligonucleotides, including those comprising various moieties.

### Example 8. Preparation of WV-2652

To the corresponding oligonucleotide (5′-T*fA*mGfC*mUfU*mCfU*mUfG*mUfC*mCfA*mG*fC*mU*fU*mU*mUmU-3′ (SEQ ID NO: 1943)) attached to CPG (120 mg, 71 umol/g loading), 1 M diphenyl phosphite in dry pyridine (5 mL) was added then mixed at rt for 30 min. The support was washed with dry ACN (5 mL×3), followed by drying under vacuum. The dried support was then treated with pyridine-H₂O (1:1, v/v) (5 mL) with syringe and mixed at rt for 2 h, followed by washing with dry ACN (5 mL×5) and drying under vacuum. Aliquot of CPG (1 umol, 14.1 mg) was treated with AMA (400 uL) at 35° C. for 2 h. The mixture was isolated by IEX-purification to give WV-2652 (MW: 6968.9; MS Obs. 6966.7).

### Example 9. Preparation of WV-2653

To the corresponding oligonucleotide (5′-T*fA*mGfC*mUfU*mCfU*mUfG*mUfC*mCfA*mG*fC*mU*fU*mU*mUmU-3′ (SEQ ID NO: 1944)) attached to CPG (120 mg, 71 umol/g loading), 1 M diphenyl phosphite in dry pyridine (5 mL) was added then mixed at rt for 30 min. The support was washed with dry ACN (5 mL×3), followed by drying under vacuum. The dried support was then treated with pyridine-H₂O (1:1, v/v) (5 mL) with syringe and mixed at rt for 2 h, followed by washing with dry ACN (5 mL×5) and drying under vacuum. Aliquot of CPG (1 umol, 14.1 mg) was treated with 0.15 M 3-Phenyl-1,2,4-dithiazoline-5-one in BSA-ACN (1:9, v/v) for 1 h. The support was mixed with AMA (400 uL) at 35° C. for 2 h. The mixture was isolated by IEX-purification to give WV-2653 (MW: 7000.9; MS Obs. 6997.3).

### Example 10. Preparation of WV-2654

The corresponding oligonucleotide (5′-T*fA*mGfC*mUfU*mCfU*mUfG*mUfC*mCfA*mG*fC*mU*fU*mU*mUmU-3′(SEQ ID NO: 1945)) attached to CPG (1 umol, 14.1 mg) was treated with 0.1 M n-Pr phosphoramidite (Phosphoramidous acid, N,N-bis(1-methylethyl)-, propyl 2-cyanoethyl ester) and 0.5 M ETT in ACN for 15 min, followed by the treatment with 1.1 M TBHP in decan-DCM (1:4, v/v) for 15 min. The support was mixed with AMA (400 uL) at 35° C. for 2 h. The mixture was isolated by IEX-purification to give WV-2654 (MW: 7026.9; MS Obs. 7022.0).

### Example 11. Preparation of WV-2655

The corresponding oligonucleotide (5′-T*fA*mGfC*mUfU*mCfU*mUfG*mUfC*mCfA*mG*fC*mU*fU*mU*mUmU-3′ (SEQ ID NO: 1946)) attached to CPG (1 umol, 14.1 mg) was treated with 0.1 M n-Pr phosphoramidite (Phosphoramidous acid, N,N-bis(1-methylethyl)-, propyl 2-cyanoethyl ester) and 0.5 M ETT in ACN for 15 min, followed by the treatment with 0.15 M 3-Phenyl-1,2,4-dithiazoline-5-one in ACN for 15 min. The support was mixed with AMA (400 uL) at 35° C. for 2 h. The mixture was isolated by IEX-purification to give WV-2655 (MW: 7043.0; Obs. 7040.9).

### Example 12. Preparation of WV-2656

To the corresponding oligonucleotide (5′-fA*mGfC*mUfU*mCfU*mUfG*mU fC*mCfA*mG*fC*mU*fU*mU*mUmU-3′ (SEQ ID NO: 1947)) attached to CPG (120 mg, 71 umol/g loading), 0.1 M Dimethyl C3 phosphoroamidite (Phosphoramidous acid, N,N-bis(1-methylethyl)-, 3-[bis(4-methoxyphenyl)phenylmethoxy]-2,2-dimethylpropyl 2-cyanoethyl ester) and 0.5 M ETT in dry ACN (5 mL) was added and mixed at rt for 15 min. The support was washed with dry ACN (5 mL×3). To the support, 0.1 M 1,2,4-dithiazole-5-thione in dry pyridine (5 mL) was added then mixed at rt for 15 min. The support was washed with dry ACN (5 mL×3). To the support, 3% TCA in DCM (5 mL) was added in continuous flow at rt for 2 min. The support was washed with dry ACN (5 mL×3). To the aliquot of CPG (2 umol, 28.1 mg), 1 M diphenyl phosphite in dry pyridine (1 mL) was added then mixed at rt for 30 min. The support was washed with dry ACN (2 mL×3), followed by drying under vacuum. The dried support was then treated with pyridine-H₂O (1:1, v/v) (1 mL) with syringe and mixed at rt for 2 h, followed by washing with dry ACN (5 mL×5) and drying under vacuum. The support was mixed with AMA (400 uL) at 35° C. for 2 h. The mixture was isolated by IEX-purification to give WV-2656 (MW: 6830.8; MS Obs. 6831.1).

### Example 13. Preparation of WV-2657

To the corresponding oligonucleotide (5′-fA*mGfC*mUfU*mCfU*mUfG*mU fC*mCfA*mG*fC*mU*fU*mU*mUmU-3′ (SEQ ID NO: 1948)) attached to CPG (120 mg, 71 umol/g loading), 0.1 M Dimethyl C3 phosphoroamidite (Phosphoramidous acid, N,N-bis(1-methylethyl)-, 3-[bis(4-methoxyphenyl)phenylmethoxy]-2,2-dimethylpropyl 2-cyanoethyl ester) and 0.5 M ETT in dry ACN (5 mL) was added and mixed at rt for 15 min. The support was washed with dry ACN (5 mL×3). To the support, 0.1 M 1,2,4-dithiazole-5-thione in dry pyridine (5 mL) was added then mixed at rt for 15 min. The support was washed with dry ACN (5 mL×3). To the support, 3% TCA in DCM (5 mL) was added in continuous flow at rt for 2 min. The support was washed with dry ACN (5 mL×3). To the aliquot of CPG (2 umol, 28.1 mg), 1 M diphenyl phosphite in dry pyridine (1 mL) was added then mixed at rt for 30 min. The support was washed with dry ACN (2 mL×3), followed by drying under vacuum. The dried support was then treated with pyridine-H₂O (1:1, v/v) (1 mL) with syringe and mixed at rt for 2 h, followed by washing with dry ACN (5 mL×5) and drying under vacuum. The support was treated with 0.15 M (1S)-(+)-(10—Camphorsulfonyl)-oxaziridine in BSA-ACN (1:9, v/v) for 1 h. The support was mixed with AMA (400 uL) at 35° C. for 2 h. The mixture was isolated by IEX-purification to give WV-2657 (MW: 6846.8; MS Obs. 6844.7).

### Example 14. Preparation of WV-2658

To the corresponding oligonucleotide (5′-fA*mGfC*mUfU*mCfU*mUfG*mUfC*mCfA*mG*fC*mU*fU*mU*mUmU-3′ (SEQ ID NO: 1949)) attached to CPG (120 mg, 71 umol/g loading), 0.1 M Dimethyl C3 phosphoroamidite (Phosphoramidous acid, N,N-bis(1-methylethyl)-, 3-[bis(4-methoxyphenyl)phenylmethoxy]-2,2-dimethylpropyl 2-cyanoethyl ester) and 0.5 M ETT in dry ACN (5 mL) was added and mixed at rt for 15 min. The support was washed with dry ACN (5 mL×3). To the support, 0.1 M 1,2,4-dithiazole-5-thione in dry pyridine (5 mL) was added then mixed at rt for 15 min. The support was washed with dry ACN (5 mL×3). To the support, 3% TCA in DCM (5 mL) was added in continuous flow at rt for 2 min. The support was washed with dry ACN (5 mL×3). To the aliquot of CPG (2 umol, 28.1 mg), 1 M diphenyl phosphite in dry pyridine (1 mL) was added then mixed at rt for 30 min. The support was washed with dry ACN (2 mL×3), followed by drying under vacuum. The dried support was then treated with pyridine-H₂O (1:1, v/v) (1 mL) with syringe and mixed at rt for 2 h, followed by washing with dry ACN (5 mL×5) and drying under vacuum. The support was treated with 0.15 M 3-Phenyl-1,2,4-dithiazoline-5-one in BSA-ACN (1:9, v/v) for 15 min. The support was mixed with AMA (400 uL) at 35° C. for 2 h. The mixture was isolated by IEX-purification to give WV-2658 (MW 6862.9; MS Obs. 6860.7).

### Example 15. Preparation of WV-3122

Oligonucleotide WV-3122 was synthesized at a scale of 50 umol using standard cyanoethyl phosphoramidite chemistry up through the final T base and was left on CPG support with the DMT protecting group on (5′-T*fG*mUfC*mCfA*mGfC*mUfU*mUfA*mUfU*mG fGmGfAmG*T*mU-3′ (SEQ ID NO: 1950)). The final phosphate (PO) was then added to the 5′ end of the oligonucleotide on the synthesizer. Briefly, the DMT protecting group was removed using 3% trichloroacetic acid in dichloromethane. During the coupling step, equal volumes of bis-cyanoethyl-N,N-diisopropyl CED phosphoramidite (0.1M in acetonitrile, ChemGenes Corporation catalog No. CLP-1454) and 5-ethylthio tetrazole (0.5M in acetonitrile) were added with a contact time of 5 min. The coupling step was repeated. Oxidation was performed using 0.02M iodine in tetrahydrofuran/pyridine/water. The oligonucleotide was deprotected by first washing with 20% diethylamine in acetonitrile on support for 15 minutes. The support was washed with acetonitrile and dried. The oligonucleotide was then cleaved and further deprotected using ammonium hydroxide/ethanol (3:1 v/v) at 50° C. overnight. Target Mass 7062.0; Observed 7062.4.

### Example 16. Preparation of WV-7645

Oligonucleotide WV-7645 was synthesized at a scale of 50 umol using standard cyanoethyl phosphoramidite chemistry up to the penultimate base (fG)and was left on CPG support with the DMT protecting group on (5′-fG*mUfC*mCfA*mGfC*mUfU*mUfA*mUfU*mG*fG*mG*fA*mG*fG*mC*T*mU-3′ (SEQ ID NO: 1951)). The final base (5MRdT) was then added to the 5′ end of the oligonucleotide on the synthesizer using standard coupling conditions. Briefly, the DMT protecting group was removed using 3% trichloroacetic acid in dichloromethane. During the coupling step, equal volumes of 5′-(R)—C-Me-S′-DMT-dT-CNE phosphoramidite (0.1 M in acetonitrile) and 5-ethylthio tetrazole (0.5 M in acetonitrile) were added with a contact time of 5 min. The coupling step was repeated. Sulfurization was performed using 0.1M DDTT in pyridine. The final phosphate (PO) was then added to the 5′ end of the oligo on the synthesizer. The DMT protecting group was removed using 3% trichloroacetic acid in dichloromethane. During the coupling step, equal volumes of bis-cyanoethyl-N,N-diisopropyl CED phosphoramidite (0.1 M in acetonitrile, ChemGenes Corporation catalog No. CLP-1454) and 5-ethylthio tetrazole (0.5 M in acetonitrile) were added with a contact time of 5 min. The coupling step was repeated. Oxidation was performed using 0.02 M iodine in tetrahydrofuran/pyridine/water. The oligonucleotide was deprotected by first washing with 20% diethylamine in acetonitrile on support for 10 minutes. The support was washed with acetonitrile and dried. The oligonucleotide was then cleaved and further deprotected using ammonium hydroxide at 40° C. overnight, giving ca. 31 mg crude at 68% purity. The crude product was further purified to provide the final product. MW: 7839.6. MS Observed: 7838.6.

### Example 17. Example Procedure for Incorporation of Amino Linker

Oligonucleotide WV-3973 was synthesized at a scale of 50 umol using standard cyanoethyl phosphoramidite chemistry up through the final Aeo base leaving the DMT protecting group on (5′-Aeo*Geo*m5Ceo*Teo*Teo*C*T*T*G*T*C*C*A*G*C*Teo*Teo*Teo*Aeo*Teo-3′ (SEQ ID NO: 1952)). The amine linker was then added to the 5′ end of the oligo on the synthesizer. Briefly, the DMT protecting group was removed using 3% trichloroacetic acid in dichloromethane. During the coupling step, equal volumes of TFA-amino C₆ CED phosphoramidite (0.15M, ChemGenes Corporation catalog no. CLP-1553 or Glen Research catalog no. 10-1916) and 5-ethylthio tetrazole (0.5M in acetonitrile) were added with a contact time of 5 min. The coupling step was repeated. Oxidation was performed using 0.02M iodine in tetrahydrofuran/pyridine/water. The oligonucleotide was deprotected by first washing with 20% diethylamine in acetonitrile on support for 15 minutes. The support was washed with acetonitrile and dried. The oligo was then cleaved and further deprotected using ammonium hydroxide at 50° C. overnight. Target Mass: 7288.0. Observed: 7286.3.

### Example 18. Example Procedure for Incorporation of Carbohydrate Moieties in Solution Phase—Preparation of WV-5287

A solution of a carbohydrate-containing carboxylic acid compound (2 equivalent), HATU (1.8 equivalent) and diisopropylethylamine (8 equivalent) in dry acetonitrile (or dry DMF) was vortexed for 2 minutes. To this solution was added a solution of oligonucleotide (1 equivalent) in water. Reaction mixture was vortexed for 2 minutes and kept for 60 minutes. By this time the reaction typically went to completion. The solvent was removed under vacuum and diluted with water appropriately and purified by RP column chromatography or IEX chromatography. In case carbohydrate (e.g., GalNAc) moieties were protected as acetates, NH3 treatment was performed before purification. An example is described below.

### Preparation of WV-5287—Example Incorporation of Carbohydrate Moieties by Conjugation with WV-2422

A solution of a carboxylic acid containing three carbohydrate moieties (see scheme below, 17 mg, 10.8 umol), HATU (3.7 mg, 9.72 umol), and DIPEA (8 ul, 43.2 umol) was thoroughly vortexed for 2 minutes in 3 mL dry DMF. To this solution was added WV-2422 (40.6 mg, 5.4 umol) in 1.5 mL water and the mixture was shaken for 60 minutes. Completion of the reaction was monitored by LC-MS (˜1 hour). After the reaction was complete, solvent was removed under reduced pressure and the crude product was purified by IEX. Molecular weight calculated: 8961. Deconvoluted Mass: 8962.

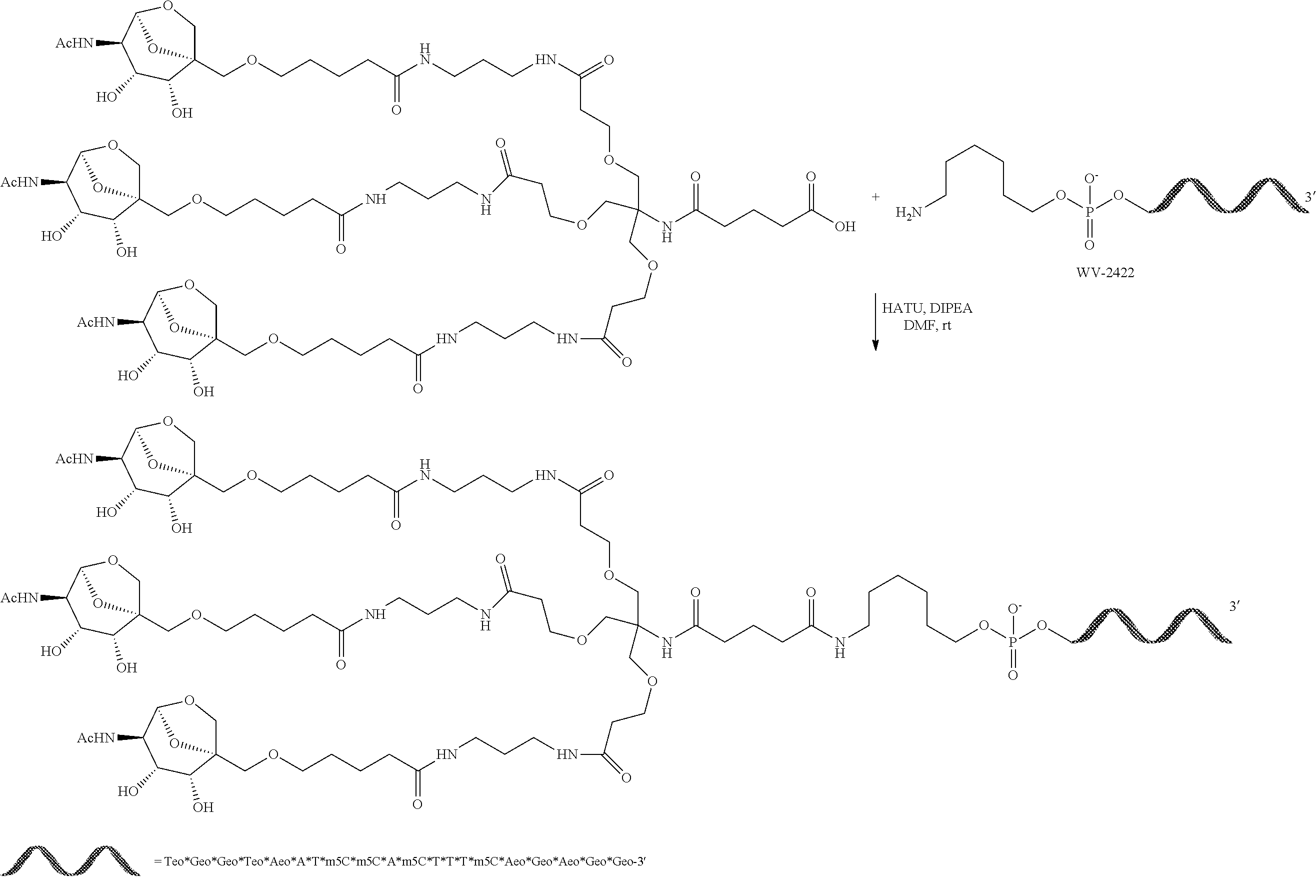

### Example 19. Example Procedure for Incorporation of Carbohydrate Moieties in Solution Phase—Preparation of WV-3969

A solution of a carboxylic acid containing GalNac moieties (38 mg, 20 umol), HATU (7 mg, 17.9 umol), and DIPEA (15 ul, 80 umol) was thoroughly vortexed for 2 minutes in 4 ml dry AcCN. To this solution was added WV-2422 (50 mg, 6.7 umol) in 2 ml water and the mixture was shaken for 60 minutes. Completion of the reaction was monitored by LC-MS (˜1 hour). After the reaction was complete, solvent was removed under reduced pressure. The crude product was dissolved in 30% ammonia solution and heated at 50° C. for three hours and the solvent was removed under vacuum. Crude product obtained was purified by IEX. Molecular weight calculated: 9025. Deconvoluted Mass: 9024.

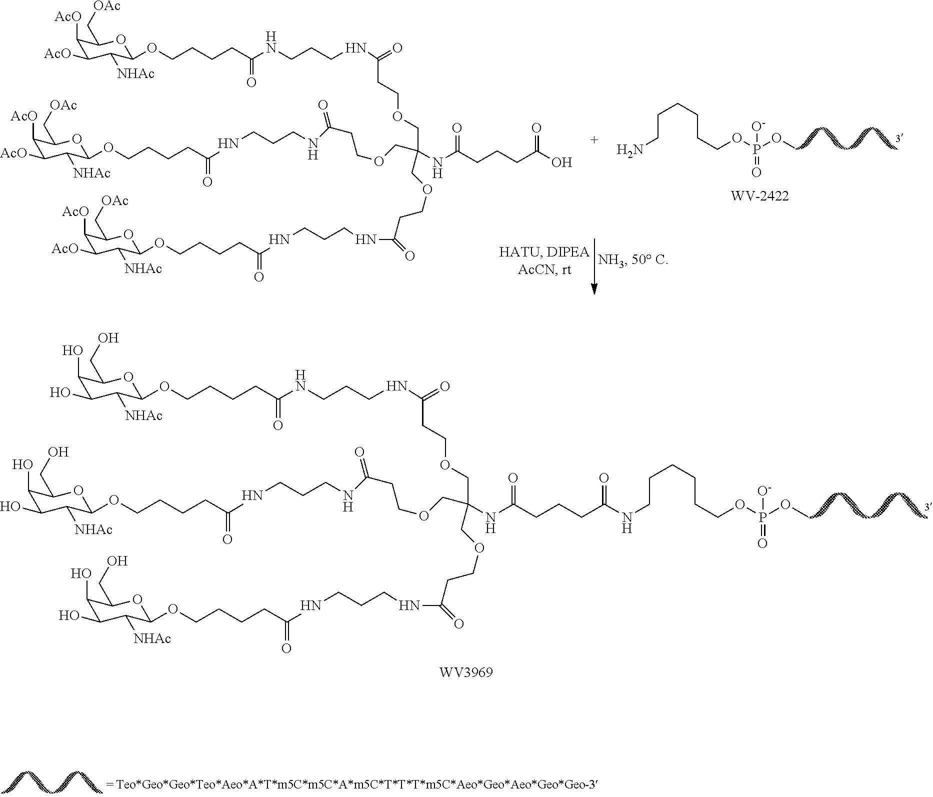 [SMILES: CC(=O)NC1C(OCCCCC(=O)NCCCNC(=O)CCOCC(COCCC(=O)NCCCNC(=O)CCCCOC2OC(COC(C)=O)C(OC(C)=O)C(OC(C)=O)C2NC(C)=O)(COCCC(=O)NCCCNC(=O)CCCCOC2OC(COC(C)=O)C(OC(C)=O)C(OC(C)=O)C2NC(C)=O)NC(=O)CCCC(=O)O)OC(COC(C)=O)C(OC(C)=O)C1OC(C)=O ; COP(=O)([O-])OCCCCCCN ; COP(=O)([O-])OCCCCCCNC(=O)CCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

### Example 20. Preparation of WV-8095

Synthesis of WV-8095 was performed iteratively on an ÄKTA OP100 synthesizer (GE healthcare) using a 12-mL stainless steel column reactor on a 250 umol scale using NittoPhase HL (Loading 190 umol/g, Kinovate Life Sciences). During synthesis, chain elongation consisted of four steps namely detritylation, coupling, oxidation/thiolation and capping. Detritylation was performed using 3% DCA in toluene with a UV watch command set at 436 nm. Following detritylation, 4 CV of ACN was used to wash off the detritylation reagent. Coupling was performed using 0.175 M amidite solutions in ACN and 0.6M CMIMT in ACN for stereo-defined monomers and 0.6 M ETT for standard amidites. All phosphoramidite and ETT solutions were prepared and dried over 3A molecular sieves for at least 3 hours prior to synthesis. The CMIMT solution was dried over Trap Pak sieves (Bioautomation) for 90 minutes prior to use. Coupling was performed by mixing 40% (by volume) of amidite solution with 60% of the activator in-line prior to addition to the column. The coupling mixture was then recirculated for a minimum of 8 minutes to enhance the coupling efficiency. Following coupling, the column was washed with no less than 2CV of ACN. The column was then treated with Capping B solution (acetic anhydride, lutidine, ACN) mixture for 2 CV to acetylate the chiral axillary amine for stereo-defined couplings. Following this step the column was washed with ACN for at least 2 CV. Thiolation was then performed with 0.1 M POS in ACN with a contact time of 6 min for 2 CV. After a 2 CV thio wash step using ACN, capping was performed using 0.5 CV of Capping A (20% N-methylimidazole in Acetonitrile (ACN)) and Capping B reagents mixed inline (1:1) followed by a 2 CV ACN wash. For cycles to form natural phosphate linkages, oxidation was performed using 1.1 M TBHP solution (in DCM and decane) for 2 min and 77 equivalents.

Cleavage and Deprotection of WV-8095: The DPSE auxiliary on WV-8095 were removed by treating the oligonucleotide bound solid support with a 1M solution of TEA.HF made by mixing TEA.3HF, TEA, DMF, and water in a v/v ratio of 10:9:61:20. The mixture was then heated at 50° C. for 90 min. The mixture was cooled (ice bath) and then filtered (10 micron). The cake was washed with ACN and water and dried under vacuum. The dry cake was then taken up in ammonia: methylamine mixture (1:1, 20 mL) and the mixture shaken at room temperature for 3.0 hrs. The mixture was then filtered (10 micron) and the cake washed with water (3×20 mL). The filtrate liquor was obtained and analyzed by UPLC and a purity of 23.69% FLP found. The mixture was then neutralized with acetic acid to a pH value 6.1. Quantitation was done using a Nano Drop one spectrophotometer (Thermo Scientific) and a yield of about 21,000 ODs obtained.

Purification of WV-8095: The crude WV-8095 was desalted on a 2K generated cellulose membrane until the conductivity was ≤3 mScm⁻¹. The desalted material (142 mL) was then diluted with 20 mM NaOH to 250 mL and loaded on to a Waters AP2 column (2 cm×20 cm) packed with TSKgel SuperQ 5PW (Tosoh Biosciences). Purification was performed on an ÄKTA 100 Explorer (GE Healthcare) using 20 mM NaOH and 2.5 M NaCl as eluents. Fractions were analyzed for purity. In some embodiments, purification after this initial purification may not be as high (for example, the pooled fraction having a purity of about 65%). If desired, further purification can be performed to increase purity. For example, a pooled fraction (162 mL, 7785 OD) was diluted to 1500 mL and re-purified on Source 15Q using the conditions described above, increasing the purity from about 65% to >83% with a yield of 2385 OD.

Desalting of WV-8095: The purified WV-8095 sample (2385 OD) was then desalted on 2K generated cellulose membrane with no material loss. The desalted material was then lyophilized to obtain 75 mg of WV-8095 as a white powder. MW (Calc.): 7953.95; MS (Found): 7953.2.

### Example 21. Preparation of WV-8061

Triantennay GalNAc (30.4 mg, 1.6 eq), and HATU (5.44 mg, 1.5 eq.) were transferred into a 50-mL plastic tube. Anhydrous acetonitrile (1.5 mL) was then added to the tube to dissolve the mixture. This was followed by the addition of DIPEA (d=0.742, 16.86 uL, 10 eq) into the tube. The mixture was then shaken for 10 min at room temperature. This mixture was then added to WV-8095 (75 mg) dissolved in water (3.0 mL) and the mixture was shaken for 60 min at 37° C. The progress of the reaction was monitored by UPLC. It was found that the reaction was complete after 1h. The reaction mixture was concentrated under vacuum (by speed vac) to remove acetonitrile. The resultant GalNAc-conjugated oligo WV-8061 was then treated with conc. ammonium hydroxide (2 mL) for 1 h at 37° C. The formation of the final product was monitored by UPLC. The ammonia hydroxide in the sample was evaporated under vacuum (by speed vac) overnight. The conjugated samples were dissolved in water and purified by reversed phase HPLC. Following purification the material was desalted and lyophilized to obtain WV-8061 with a yield of 1400 OD. MW (Calc.): 9562.8; MS (Found): 9561.7.

### Example 22. Preparation of WV-8094—Example Vinyl Phosphonate Deprotection

DMT-5′-VP-dT amidite was utilized to incorporate 5′-VP-dT. To deprotect the 5′—VP group, after preparation of the oligonucleotide chain, into a plastic container with the oligonucleotide bound support (250 umol), a bright yellow mixture of TMSI, DCM and pyridine (125 mL) in the v/v ratio of 3:96:2 was added and the mixture shaken at room temperature for 30 min. The TMSI solution was then decanted and the solid support treated with a mixture of 2-Dodecane Thiol, TEA and ACN (125 mL×3) in the v/v ratio of 24:38:38. A final charge of the 2-Dodecane mixture was added and allowed to stand for 45 min. The mixture was then filtered, the support washed with ACN (50 mL×3) and then standard cleavage and base deprotection performed as described for WV-8095. MW (Calc.): 8028.9; MS (Found): 8030.2.

### Example 23. Preparation of 5′-triazole

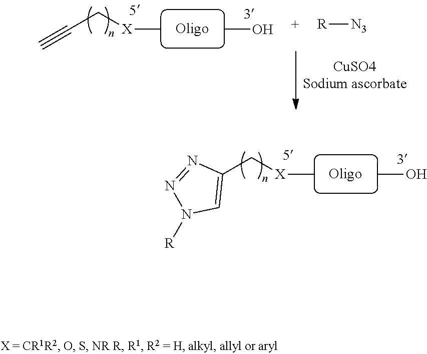

A mixture of the corresponding azide (1.5 μmol), 5′-alkynyl oligonucleotide (1 μmol), CuSO₄ (10 μmop sodium ascorbate (100 μmop and trishydroxypropyltriazole (70 μmop in 0.2 M NaCl solution is stirred for 1 hour. After completion of reaction, purification of the crude product is done by RP-HPLC or IEX-HPLC. After HPLC purification, pure fractions are combined and solvent is removed under vacuum. Residue is dissolved in water, desalted and lyophilized to give the product.

In some embodiments, n is 0-10. In some embodiments, n is 1-10. In some embodiments, X is —C(R¹)(R²)—, wherein each of R¹ and R² is independently R. In some embodiments, X is —O—. In some embodiments, X is —S—. In some embodiments, X is —NR—. In some embodiments, each of R, R¹ and R² is independently —H, or an optionally substituted group selected from alkyl, allyl and aryl. In some embodiments, each of R, R¹ and R² is independently —H, or an optionally substituted group selected from C₁₋₁₀ alkyl, C₃₋₁₀ allyl and C₆₋₁₀ aryl. In some embodiments, R is —H. In some embodiments, R¹ is —H. In some embodiments, R² is —H.

### Example 24. Provided Technologies Provide High Activities

For ssRNAi, it has been widely accepted prior to the present disclosure that 5′-phosphorylation is required for activity. In some embodiments, the present disclosure, surprisingly, demonstrated that with features provided in the present disclosure, oligonucleotides without 5′-phosphorylation can deliver activities comparable to, or higher than, oligonucleotides of traditional ssRNAi designs with 5′-phosphorylation. For examples, see data provided in the Tables.

### Example 25. Provided 5′-End Structures Provide High Activities

In some embodiments, the present disclosure provides various 5′-end structures. Among other things, the present disclosure demonstrated that oligonucleotides comprising provided 5′-end modifications can be highly active and/or stable, for example, when used as ssRNAi reagents. For example, as shown in Table 46, provided oligonucleotide WV-7645 demonstrated surprisingly high activity, with an IC50 as low as 7 pM.

### Example 26. Provided Technologies Provide High Activity and/or Selectivity/Specificity

Among other things, the present disclosure provides technologies that can achieve high activity and/or selectivity/specificity, including knockdown of target gene expression (e.g., mRNA level) and/or target protein level. As demonstrated herein, including, but not limited to, the data in the Tables below, provided technologies can selectively reduce the level of one mRNA but not a homologous mRNA which differs from it by only one base. Thus, in some embodiments, the present disclosure provides technologies that can achieve allele-specific reduction of levels of transcripts/products of a disease-associated allele, while maintaining levels of transcripts/products of a normal allele; see, e.g., Table 61. Oligonucleotides are described in detail in Table 1A.

Table 12. Table 12 shows the IC₅₀ for different single-stranded RNAi agents, which target APOC3. Tested oligonucleotides are: WV-1275, WV-1277, WV-1828, WV-1829, WV-1830 and WV-1831. CI, confidence interval. Cells used were Hep3B, and oligonucleotides for APOC3 were delivered using Lipofectamine® 2000 transfection reagent (ThermoFisher, Grand Island, N.Y.).

TABLE 12
Activity of APOC3 oligonucleotides.

| Oligonucleotide | IC50 (nM) |
|---|---|
| WV-1275 | 1.35 |
| WV-1277 | 1.94 |
| WV-1828 | 0.43 |
| WV-1829 | 0.67 |
| WV-1830 | 0.689 |
| WV-1831 | 0.986 |

Table 13. Table 13 shows the in vitro potency for different single-stranded RNAi agents, which target APOC3. Tested oligonucleotides are: WV-2110, WV-3068, WV-2817, WV-2818, WV-2720, WV-2721, and WV-3021.

TABLE 13
Activity of APOC3 oligonucleotides.

| Conc. (exp | | | | | | | | |
|---|---|---|---|---|---|---|---|---|
| 10) (nM) | WV-2110 | | WV-3068 | | WV-2817 | | WV-2818 | |
| 1.398 | 0.254 | 0.263 | 0.085 | 0.089 | 0.276 | 0.224 | 0.176 | 0.219 |
| 0.792 | 0.308 | 0.288 | 0.094 | 0.084 | 0.219 | 0.187 | 0.155 | 0.121 |
| 0.204 | 0.563 | 0.587 | 0.203 | 0.179 | 0.280 | 0.390 | 0.171 | 0.181 |
| −0.398 | 0.797 | 0.848 | 0.382 | 0.387 | 0.515 | 0.508 | 0.409 | 0.374 |
| −1.000 | 0.988 | 1.037 | 0.595 | 0.629 | 0.791 | 0.713 | 0.595 | 0.634 |
| −1.602 | 1.008 | 1.015 | 0.884 | 0.830 | 0.994 | 1.001 | 0.921 | 0.896 |
| −2.204 | 0.909 | 0.884 | 0.934 | 0.890 | 1.015 | 1.022 | 0.941 | 0.988 |
| −2.824 | 0.941 | 1.081 | 0.842 | 0.954 | 0.842 | 0.902 | 0.836 | 0.902 |

| Conc. (exp | | | | | | |
|---|---|---|---|---|---|---|
| 10) (nM) | WV-2720 | | WV-2721 | | WV-3021 | |
| 1.398 | 0.328 | 0.330 | 0.328 | 0.272 | 0.265 | 0.205 |
| 0.792 | 0.356 | 0.304 | 0.337 | 0.298 | 0.199 | 0.144 |
| 0.204 | 0.490 | 0.508 | 0.571 | 0.522 | 0.245 | 0.229 |
| −0.398 | 0.890 | 0.842 | 0.842 | 0.769 | 0.424 | 0.415 |
| −1.000 | 0.921 | 0.947 | 0.915 | 0.915 | 0.718 | 0.661 |
| −1.602 | 1.051 | 1.015 | 1.073 | 0.974 | 0.941 | 0.866 |
| −2.204 | 1.066 | 1.073 | 1.066 | 0.915 | 1.051 | 0.902 |
| −2.824 | 0.915 | 1.022 | 0.830 | 0.981 | 0.860 | 0.988 |

In Tables 13, 14, 20 and others: the Conc. in the first column is presented as an exponent (exp) of 10; e.g., 1.398 indicates 10^{1.398} nM. In various columns, data from replicate experiments is shown. Numbers indicate residual mRNA level (e.g., 0.254 in column 2, row 2 represents 25.4% residual mRNA level relative to control, or 74.6% knockdown).

Table 14. Table 14 shows the in vitro potency different single-stranded RNAi agents, which target APOC3. Tested oligonucleotides are: WV-2817 and WV-3021.

TABLE 14
Activity of APOC3 oligonucleotides.

| | | |
|---|---|---|
| Conc. (exp | | |
| 10) (nM) | WV-2817 | WV-3021 |

| | | | | |
|---|---|---|---|---|
| 1.398 | 0.276 | 0.224 | 0.265 | 0.205 |
| 0.792 | 0.219 | 0.187 | 0.199 | 0.144 |
| 0.204 | 0.280 | 0.390 | 0.245 | 0.229 |
| −0.398 | 0.515 | 0.508 | 0.424 | 0.415 |
| −1.000 | 0.791 | 0.713 | 0.718 | 0.661 |
| −1.602 | 0.994 | 1.001 | 0.941 | 0.866 |
| −2.204 | 1.015 | 1.022 | 1.051 | 0.902 |
| −2.824 | 0.842 | 0.902 | 0.860 | 0.988 |

Table 15. Table 15 shows in vitro potency, including knockdown of mRNA (Table 15A) and protein levels (Table 15B) of APOC3, for two single-stranded RNAi agents: WV-1868 and WV-2110.

TABLE 15A
Activity of APOC3 oligonucleotides.

| | | |
|---|---|---|
| Conc. (nM) | WV-1868 | WV-2110 |

| | | | | |
|---|---|---|---|---|
| 0 | 100.000 | 100.000 | 100.000 | 100.000 |
| 0.1 | 101.965 | 112.356 | 91.261 | 78.899 |
| 0.4 | 97.136 | 81.117 | 64.086 | 66.807 |
| 1.6 | 62.767 | 64.531 | 33.173 | 32.043 |
| 6.2 | 32.043 | 29.282 | 19.725 | 19.053 |
| 25 | 15.692 | 11.810 | 19.862 | 20.279 |

In Tables 15 and others: the Conc. in the first column is presented in nM. In various columns, data from replicate experiments are shown. In Tables 15 and others wherein the highest activity number is around 100: Numbers indicate residual mRNA level, e.g., 100.000 in column 2, row 2 represents 100.000% residual mRNA level relative to control, or 0% knockdown; and 0.000 would indicate 0% residual mRNA level or 100% knockdown

TABLE 15A
Activity of APOC3 oligonucleotides.

| Conc. (nM) | WV-1868 | | WV-2110 | |
|---|---|---|---|---|
| 0 | 96.73199783 | 103.268 | 104.7948 | 95.2052 |
| 0.1 | 98.21632145 | 85.76992 | 82.96123 | 82.66324 |
| 0.4 | 65.44366917 | 64.85707 | 41.62211 | 43.6669 |
| 1.6 | 24.87594999 | 23.73695 | 12.00211 | 12.84661 |
| 6.2 | 0.022395917 | 0.022396 | 0.242648 | 1.803896 |
| 25 | 0.022396 | 0.022396 | 0.242648 | 2.337392 |

Table 16. Table 16 shows the IC₅₀ for different single-stranded RNAi agents, which all target the same sequence in APOC3, in Hep3B cells. Tested oligonucleotides are: WV-3068, WV-2818, WV-2817, WV-2721, WV-2720, WV-2110, and WV-3021. Results of two experiments are shown.

TABLE 16
Activity of APOC3 oligonucleotides.

| Oligonucleotide | Experiment 1: IC50 (nM) | Experiment 2: IC50 (nM) |
|---|---|---|
| WV-1868 | 1.90 | 0.578 |
| WV-2110 | 0.583 | 0.172 |
| WV-3068 | 0.318 | 0.051 |
| WV-2817 | 0.182 | 0.039 |
| WV-2818 | 0.317 | 0.059 |
| WV-2720 | 0.555 | 0.173 |
| WV-2721 | 1.15 | 0.432 |
| WV-3021 | 0.115 | 0.021 |

Table 17. Table 17 shows the IC₅₀ and 95% CI (confidence interval) (pM) for different single-stranded RNAi agents, which both target the same sequence in APOC3. Tested oligonucleotides are: WV-1307 and WV-1308.

TABLE 17
Activity of APOC3 oligonucleotides.

| Oligonucleotide | IC 50 (pM) | 95% CI (pM) |
|---|---|---|
| WV-1307 | 1581 | 634 to 3941 |
| WV-1308 | 235 | 192 to 288 |

Table 18. Table 18 shows the IC₅₀ and 95% CI (pM) for different single-stranded RNAi agents, which target the same sequence in APOC3. Tested oligonucleotides are: WV-2134, WV-1308, and WV-2420. WV-2134 is an antisense oligonucleotide which knocks down the target via RNase H-mediated knockdown; WV-1308 and WV-2420 are single-stranded RNAi agents.

TABLE 18
Activity of APOC3 oligonucleotides.

| Oligonucleotide | IC 50 (pM) | 95% CI (pM) |
|---|---|---|
| WV-2134 | 815 | 521 to 2798 |
| WV-1308 | 197 | 353 to 1716 |
| WV-2420 | 51 | 90 to 233 |

Table 19. Table 19 shows the IC₅₀ for different single-stranded RNAi agents, which target the same sequence in APOC3, in in Hep3B cells (tested at 48 hours). Tested oligonucleotides are: WV-2110, WV-2716, WV-2717, WV-2718, and WV-2719.

TABLE 19
Activity of APOC3 oligonucleotides.

| Oligonucleotide | IC 50 (pM) |
|---|---|
| WV-2110 | 160 |
| WV-2716 | 1456 |
| WV-2717 | 1841 |
| WV-2718 | 4585 |
| WV-2719 | 5645 |

Table 20. Table 20 shows the in vitro potency for different single-stranded RNAi agents, which target the same sequence in APOC3, in in Hep3B cells (tested at 48 hours). Tested oligonucleotides are: WV-2110, WV-2712, WV-2713, WV-2714, and WV-2715. IC50 of various oligonucleotides: WV-2110, 160 pM; WV-2712, 1743 pM; and WV-2713, 950 pM.

TABLE 20
Activity of APOC3 oligonucleotides.

| Conc. (exp | | | | | | |
|---|---|---|---|---|---|---|
| 10) (nM) | WV-2110 | | WV-2712 | | WV-2713 | |
| 1.398 | 0.260 | 0.269 | 0.136 | 0.171 | 0.097 | 0.166 |
| 0.792 | 0.262 | 0.228 | 0.202 | 0.310 | 0.203 | 0.224 |
| 0.204 | 0.241 | 0.248 | 0.579 | 0.684 | 0.480 | 0.442 |
| −0.398 | 0.459 | 0.453 | 0.848 | 0.961 | 0.890 | 0.808 |
| −1.000 | | 0.711 | 1.150 | 1.008 | 0.759 | |
| −1.602 | 0.900 | 1.077 | | 1.175 | | 1.119 |
| −2.204 | 0.978 | 1.077 | 0.981 | 1.008 | 1.285 | 1.303 |
| −2.824 | 1.005 | 1.005 | 1.119 | 0.961 | 0.884 | 1.088 |

| | | |
|---|---|---|
| Conc. (exp | | |
| 10) (nM) | WV-2714 | WV-2715 |

| | | | | |
|---|---|---|---|---|
| 1.398 | 0.903 | 0.842 | 0.981 | 0.764 |
| 0.792 | 1.066 | 1.081 | 0.988 | 1.066 |
| 0.204 | | 1.166 | 1.008 | 1.166 |
| −0.398 | 1.088 | 1.285 | 1.208 | 1.233 |
| −1.000 | 1.023 | 1.119 | 1.368 | 1.259 |
| −1.602 | 0.967 | 1.378 | 1.030 | 1.191 |
| −2.204 | 0.988 | 1.135 | 1.015 | 1.191 |
| −2.824 | 0.974 | 1.023 | 0.866 | 0.941 |

Table 21. Table 21 shows the IC₅₀ and 95% CI for different single-stranded RNAi agents, which target APOC3. Tested oligonucleotides are: WV-1868, WV-2110, and WV-2111. WV-1868 is an antisense oligonucleotide (operating through RNase H-mediated knockdown), while other tested oligonucleotides are RNAi agents.

TABLE 21
Activity of APOC3 oligonucleotides.

| Oligonucleotide | IC 50 (pM) | 95% CI (pM) |
|---|---|---|
| WV-1868 | 266 | 176 to 403 |
| WV-2110 | 64 | 37 to 110 |
| WV-2111 | 163 | 64 to 412 |

Table 22. Table 22 shows the IC₅₀ and 95% CI (pM) for different single-stranded RNAi agents, which target overlapping sequences in APOC3, in in Hep3B cells (tested at 48 hours). Tested oligonucleotides are: WV-2110, WV-2693, WV-2696, WV-2697, WV-2698, and WV-2699.

TABLE 22
Activity of APOC3 oligonucleotides.

| Conc. (exp | | | | | | | | |
|---|---|---|---|---|---|---|---|---|
| 10) (nM) | WV-2110 | | WV-2693 | | WV-2696 | | WV-2697 | |
| 1.398 | 0.313 | 0.336 | 0.345 | 0.318 | 0.368 | 0.309 | 0.542 | 0.375 |
| 0.792 | 0.301 | 0.296 | 0.286 | 0.348 | 0.307 | 0.307 | 0.370 | 0.282 |
| 0.204 | 0.322 | 0.311 | 0.341 | 0.394 | 0.413 | 0.311 | 0.440 | 0.419 |
| −0.398 | 0.499 | 0.520 | 0.437 | 0.581 | 0.495 | 0.492 | 0.710 | 0.735 |
| −1.000 | 0.756 | 0.782 | 0.788 | 0.740 | 0.662 | 0.695 | 0.886 | 0.788 |
| −1.602 | 1.011 | | 0.799 | 0.997 | 0.924 | 0.844 | 1.011 | 1.145 |
| −2.204 | | 1.047 | 0.911 | 1.122 | 0.977 | 0.821 | 1.061 | 1.186 |
| −2.824 | 0.970 | 0.983 | 0.886 | 0.937 | 1.069 | 0.893 | | 0.997 |

| Conc. (exp | | | | | | |
|---|---|---|---|---|---|---|
| 10) (nM) | WV-2697 | | WV-2698 | | WV-2699 | |
| 1.398 | 0.542 | 0.375 | 0.305 | 0.256 | 0.373 | 0.282 |
| 0.792 | 0.370 | 0.282 | 0.275 | 0.239 | 0.305 | 0.249 |
| 0.204 | 0.440 | 0.419 | 0.391 | 0.341 | 0.428 | 0.368 |
| −0.398 | 0.710 | 0.735 | 0.585 | 0.506 | 0.667 | 0.662 |
| −1.000 | 0.886 | 0.788 | | 0.761 | | 0.788 |
| −1.602 | 1.011 | 1.145 | 0.827 | 0.893 | 0.905 | 0.844 |
| −2.204 | 1.061 | 1.186 | 1.069 | 0.804 | 1.054 | |
| −2.824 | | 0.997 | 0.963 | 0.821 | 0.950 | 0.839 |

Table 23. Table 23 shows the IC₅₀ and 95% CI (pM) for different single-stranded RNAi agents, which target APOC3, in Hep3B cells (tested at 48 hours). Tested oligonucleotides are: WV-2110, WV-2154, and WV-2155. The latter two oligonucleotides comprise at 2′-deoxy T at the penultimate (second-to-last) or antepenultimate (third-to-last) nucleotide.

TABLE 23
Activity of APOC3 oligonucleotides.

| Oligonucleotide | IC 50 (pM) | 95% CI (pM) |
|---|---|---|
| WV-2110 | 64 | 37 to 110 |
| WV-2154 | 120 | 71 to 202 |
| WV-2155 | 74 | 22 to 255 |

Table 24. Table 24 shows the IC₅₀ and 95% CI for different single-stranded RNAi agents, which target APOC3, in Hep3B cells (tested at 48 hours). Tested oligonucleotides are: WV-2111, WV-2156, and WV-2157.

TABLE 24
Activity of APOC3 oligonucleotides.

| Oligonucleotide | IC 50 (pM) | 95% CI (pM) |
|---|---|---|
| WV-2111 | 163 | 63 to 416 |
| WV-2156 | 118 | 43 to 325 |
| WV-2157 | 245 | 111 to 540 |

Table 25. Table 25 shows the in vitro potency and IC₅₀ for different single-stranded RNAi agents, which target APOC3, in PCH (Primary Cyno Hepatocytes). Tested oligonucleotides are: WV-1868, WV-2110, WV-3068 and WV-3069. WV-1868 is an antisense oligonucleotide (operating through RNase H-mediated knockdown), while other tested oligonucleotides are RNAi agents. The penultimate nucleotide in WV-3068 (nucleotide 20) or WV-3069 (nucleotide 24) comprises a AMC6T (GalNAc). Gym indicates the oligonucleotide was delivered via gymnotic delivery. IC50 of various oligonucleotides: WV-1868, 4.06 nM; WV-2110, 1.58 nM; and WV-3068, 0.25 nM

TABLE 25
Activity of APOC3 oligonucleotides.

| Conc. (exp | | | | | | |
|---|---|---|---|---|---|---|
| 10) (nM) | WV-1868 | | WV-2110 | | WV-3068 | |
| 1.398 | 0.120 | 0.107 | 0.210 | 0.225 | 0.071 | 0.062 |
| 0.792 | 0.384 | 0.366 | 0.262 | 0.247 | 0.065 | 0.071 |
| 0.204 | 0.763 | 0.732 | 0.578 | 0.532 | 0.177 | 0.152 |
| −0.398 | 0.882 | 0.966 | 0.835 | 0.801 | 0.341 | 0.328 |
| −1.000 | 0.966 | 0.946 | 0.895 | 0.959 | 0.574 | 0.637 |
| −1.602 | 1.094 | 1.014 | | 0.986 | 0.858 | 0.784 |
| −2.204 | 0.870 | 0.858 | 0.858 | 0.835 | 0.823 | 0.763 |
| −2.824 | 0.914 | 0.939 | 0.933 | 0.823 | 0.818 | 0.829 |

| | | |
|---|---|---|
| Conc. (exp | | |
| 10) (nM) | WV-3068-Gym | WV-3069 |

| | | | |
|---|---|---|---|
| 1.398 | 0.594 | 0.590 | 0.170 |
| 0.792 | 0.642 | 0.655 | 0.179 |
| 0.204 | 0.752 | 0.747 | 0.381 |
| −0.398 | 0.768 | 0.707 | 0.737 |
| −1.000 | 0.702 | 0.790 | 0.742 |
| −1.602 | 0.806 | 0.841 | 0.889 |
| −2.204 | 0.823 | 0.784 | 1.000 |
| −2.824 | 0.707 | 0.758 | 0.876 |

Table 26. Table 26 shows the IC₅₀ for different single-stranded RNAi agents, which target APOC3, in Hep3B cells (tested at 48 hours). Tested oligonucleotides are: WV-1868, WV-2110, WV-3068, WV-2818, WV-2817, WV-2721, WV-2720, WV-2110, WV-3021.

TABLE 26
Activity of APOC3 oligonucleotides.

| Oligonucleotide | IC50 (nM) |
|---|---|
| WV-1868 | 1.90 |
| WV-2110 | 0.583 |
| WV-3068 | 0.318 |
| WV-2817 | 0.182 |
| WV-2818 | 0.317 |
| WV-2720 | 0.555 |
| WV-2721 | 1.15 |
| WV-3021 | 0.115 |

Table 27. Table 27 shows the IC₅₀ for different single-stranded RNAi agents, which target APOC3, in PCH (Primary Cyno Hepatocytes). Tested oligonucleotides are: WV-2110 and WV-3068; and WV-2420 and WV-2386.

TABLE 27
Activity of APOC3 oligonucleotides.

| Oligonucleotide | IC50 (nM) |
|---|---|
| WV-2110 | 1.29 |
| WV-3068 | 0.20 |
| WV-2386 | 1.60 |
| WV-2420 | 0.13 |

Table 28. Table 28 shows the IC₅₀ for different single-stranded RNAi agents, which target APOC3, in Hep3B cells (tested at 48 hours). Tested oligonucleotides are: WV-2420, WV-2652, WV-2653, and WV-2654. Various oligonucleotides comprise at the 5′ end a PO (WV-2420); PH or H-phosphonate (WV-2652); PS or phosphorothioate (WV-2653); or C3 PO (Mod022), as defined in the legend of Table 1A (WV-2654).

TABLE 28
Activity of APOC3 oligonucleotides.

| Oligonucleotide | IC 50 (pM) |
|---|---|
| WV-2420 | 50 |
| WV-2652 | 300 |
| WV-2653 | 104 |
| WV-2654 | 1000 |

Table 29. Tables 29A and 29B show the in vitro potency and IC₅₀ for different single-stranded RNAi agents, which target APOC3, in Hep3B cells (tested at 48 hours). Tested oligonucleotides are: WV-1308, WV-2114, WV-2150, WV-2151, WV-2114, WV-2152, and WV-2153.

TABLE 29A
Activity of APOC3 oligonucleotides.

| | | |
|---|---|---|
| Conc. (exp | | |
| 10) (nM) | WV-1308 | WV-2114 |

| | | | | |
|---|---|---|---|---|
| 0.792 | 0.133 | 0.124 | 0.092 | 0.087 |
| 0.204 | 0.184 | 0.164 | 0.265 | 0.256 |
| −0.398 | 0.329 | 0.326 | 0.557 | 0.538 |
| −1.000 | 0.584 | 0.622 | 0.809 | 0.826 |
| −1.602 | 0.685 | 0.867 | 0.714 | 0.936 |
| −2.204 | 0.892 | 0.844 | 0.879 | 0.826 |
| −2.807 | 0.956 | 0.936 | 0.809 | 0.962 |

TABLE 29B
Activity of APOC3 oligonucleotides.

| Oligonucleotide | IC 50 (pM) | 95% CI (pM) |
|---|---|---|
| WV-1308 | 144 | 88 to 235 |
| WV-2114 | 628 | 333 to 1183 |

In addition, in one experiment, WV-2150 and WV-2151 demonstrated in vitro activity that was similar to WV-1308 (data not shown); and in a different experiment, WV-2152 and WV-2153 demonstrated in vitro activity slightly better than that of WV-2114 (data not shown).

Table 30. Table 30 shows the in vitro potency of single-stranded RNAi agents to APOC3: WV-1275, WV-1828, and WV-1308.

TABLE 30
Activity of APOC3 oligonucleotides.

| Oligonucleotide | IC50 (nM) |
|---|---|
| WV-1275 | 1.5 |
| WV-1828 | 0.44 |
| WV-1308 | 0.067 |

Table 31. Table 31 shows the in vitro potency and IC₅₀ for different single-stranded RNAi agents, which target APOC3, in Hep3B cells (tested at 48 hours). Tested oligonucleotides are: WV-2420, WV-2655, WV-2656, WV-2657, and WV-2658. Various oligonucleotides comprise at the 5′ end a PO (WV-2420), C3 PS (Mod022*) (WV-2655), C3dimethyl PH (WV-2656), C3dimethyl PO (WV-2657), or C3dimethyl PS (WV-2658).

TABLE 31
Activity of APOC3 oligonucleotides.

| | | |
|---|---|---|
| Conc. (exp | | |
| 10) (nM) | WV-2420 | WV-2655 |

| | | | | |
|---|---|---|---|---|
| 0.792 | 0.208 | 0.119 | 0.561 | 0.568 |
| 0.204 | 0.211 | 0.186 | 0.832 | 0.782 |
| −0.398 | 0.318 | 0.282 | 1.090 | 0.886 |
| −1.000 | 0.481 | 0.458 | 0.956 | 0.923 |
| −1.602 | 0.850 | 0.798 | 0.880 | 0.787 |
| −2.204 | 0.936 | 0.949 | 1.121 | 0.923 |
| −2.806 | 1.244 | 0.949 | 0.943 | 0.911 |

| Conc. (exp | | | | | | |
|---|---|---|---|---|---|---|
| 10) (nM) | WV-2656 | | WV-2567 | | WV-2658 | |
| 0.792 | 0.917 | 0.695 | 0.667 | 0.667 | 0.541 | 0.468 |
| 0.204 | 0.969 | 0.776 | 0.793 | 0.740 | 0.776 | 0.735 |
| −0.398 | 1.032 | 0.917 | 0.956 | 0.868 | 0.793 | 0.826 |
| −1.000 | 1.083 | 0.898 | 1.025 | 0.856 | 0.930 | 0.892 |
| −1.602 | 1.400 | 1.261 | 1.553 | 1.553 | 1.699 | 1.288 |
| −2.204 | 0.862 | 0.838 | 0.874 | 0.729 | 0.880 | 0.793 |
| −2.806 | 0.963 | 0.838 | 0.917 | 0.923 | 1.003 | 0.904 |

Table 32. Table 32 shows the IC₅₀ for different single-stranded RNAi agents, which target APOC3: WV-2110, WV-3122, WV-3124 to WV-3127, and WV-3133 to WV-3137.

TABLE 32
Activity of APOC3 oligonucleotides.

| Oligonucleotide | IC50 (nM) |
|---|---|
| WV-2110 | 0.8185 |
| WV-3122 | 0.3884 |
| WV-3124 | 0.9753 |
| WV-3125 | ~1.709 |
| WV-3126 | 1.469 |
| WV-3127 | 3.466 |
| WV-3133 | 2.44 |
| WV-3134 | 6.81 |
| WV-3135 | 1.473 |
| WV-3136 | 1.176 |
| WV-3137 | 13.9 |

Table 33. Table 33 shows the in vitro potency and IC₅₀ for different single-stranded RNAi agents, which target APOC3: WV-1868, WV-2110, and WV-3068. Data from replicate experiments are shown. The penultimate nucleotide of WV-3068 comprises a TGaNC6T.

TABLE 33
Activity of APOC3 oligonucleotides.

| Oligonucleotide | IC50 (nM) | IC50 (nM) |
|---|---|---|
| WV-1868 | 4.06 | 3.36 |
| WV-2110 | 1.58 | 1.29 |
| WV-3068 | 0.24 | 0.20 |

Table 34. Table 34 shows the in vitro potency and IC₅₀ for different single-stranded RNAi agents, which target APOC3: WV-1868, WV-2110, WV-3068, WV-2817, WV-2818, WV-2720, WV-2721, and WV-3021. The penultimate nucleotide of various oligonucleotides is or comprises a 2′-deoxy T (WV-3021 and WV-2817), aminomodifier (WV-2818), or TGaNC6T (WV-3068).

TABLE 34
Activity of APOC3 oligonucleotides.

| Oligonucleotide | IC50 (nM) |
|---|---|
| WV-1868 | 3.36 |
| WV-2110 | 1.29 |
| WV-3068 | 0.20 |
| WV-2817 | 0.25 |
| WV-2818 | 0.17 |
| WV-2720 | 0.9 |
| WV-2721 | 1.06 |
| WV-3021 | 0.17 |

Table 35. Table 35 shows the in vitro potency and IC₅₀ for different single-stranded RNAi agents, which target APOC3: WV-2110, WV-3068, WV-2420, and WV-2386.

TABLE 35
Activity of APOC3 oligonucleotides.

| Oligonucleotide | IC50 (nM) |
|---|---|
| WV-2110 | 0.583 |
| WV-3068 | 0.318 |
| WV-2386 | 2.80 |
| WV-2420 | 0.27 |

Additional testing was performed on the in vitro potency for different single-stranded RNAi agents, which target APOC3: WV-3242, WV-5289, WV-5291, WV-5293, WV-5295, WV-5297, WV-5299, and WV-5301.

In one experiment, WV-2110, WV-2154 and WV-2155 all had substantial and substantially similar activity in vitro (data not shown). WV-2155 comprises a 2′-deoxy T at the antepenultimate nucleotide; WV-2154 comprises a 2′-deoxy T at the penultimate nucleotide; and WV-2110 comprises 2′-OMe at both the penultimate and antepenultimate nucleotides

In one experiment, WV-3242, WV-5289, WV-5291, WV-5293, WV-5295, WV-5297, and WV-5299 all demonstrated substantial and substantially identical activity in vitro; these various oligonucleotides all comprise a penultimate nucleotide comprising a 2′-deoxy T and have various different lengths (data not shown). WV-5301 showed some in vitro activity, though lower than the other oligonucleotides tested in that experiment.

Table 37. Table 37 shows the in vitro potency and IC₅₀ for different single-stranded RNAi agents, which target APOC3: WV-2817, WV-5288, WV-5290, WV-5292, WV-5294, and WV-5296. These various oligonucleotides comprise a 2′-deoxy T at the penultimate nucleotide and have various lengths.

TABLE 37
Activity of APOC3 oligonucleotides.

| Oligonucleotide | IC50 (nM) |
|---|---|
| WV-2817 | 0.066 |
| WV-5288 | 0.032 |
| WV-5290 | 0.003 |
| WV-5292 | 0.053 |
| WV-5294 | 0.012 |
| WV-5296 | 0.013 |
| WV-5298 | 0.015 |

Table 38. Table 38 shows the in vitro potency and IC₅₀ for different single-stranded RNAi agents, which target APOC3: WV-5290, WV-5291, WV-6431 to WV-6438, WV-6763, and WV-2477. In various oligonucleotides, the post-seed region comprises a stretch of nucleotides having patterns of modification of: mfmf, mfmfmfm, mmmmmmm, or MMMMMMM, wherein m is 2′-OMe, f is 2′-F, and M is 2′-MOE. In some oligonucleotides, the penultimate nucleotide is 2′-deoxy T.

TABLE 38
Activity of APOC3 oligonucleotides.

| Oligonucleotide | IC50 (nM) | 95% CI (nM) |
|---|---|---|
| WV-5290 | 0.106 | 0.074-0.151 |
| WV-5291 | 0.318 | 0.214-0.473 |
| WV-6431 | 0.112 | 0.063-0.202 |
| WV-6432 | 0.105 | 0.056-0.195 |
| WV-6433 | 0.145 | 0.092-0.228 |
| WV-6434 | 0.293 | 0.165-0.520 |
| WV-6435 | 0.136 | 0.091-0.204 |
| WV-6436 | 0.124 | 0.088-0.174 |
| WV-6437 | 0.209 | 0.156-0.281 |
| WV-6438 | 0.212 | 0.158-0.285 |
| WV-6763 | 0.079 | 0.062-0.100 |

Table 39. Table 39 shows the in vitro potency for different single-stranded RNAi agents, which target APOC3: WV-5291, WV-6411 to 6430, WV-6764, WV-6765, and WV-2477. Various oligonucleotides have different base sequences and different patterns of 2′-F, 2′-OMe and 2′-MOE. Some oligonucleotides comprise a 2′-deoxy T at the penultimate nucleotide.

TABLE 39
Activity of APOC3 oligonucleotides.

| nM | WV-5291 | | WV-6411 | | WV-6412 | | WV-6413 | | WV-6414 | | WV-6415 | |
|---|---|---|---|---|---|---|---|---|---|---|---|---|
| 30.0 | 8.1 | 6.2 | 25.5 | 27.5 | 25.0 | 28.3 | 23.8 | 28.8 | 27.6 | 26.1 | 27.3 | 23.3 |
| 10.0 | 19.6 | 16.5 | 39.6 | 32.1 | 38.9 | 38.0 | 38.5 | 41.1 | 41.9 | 40.9 | 39.3 | 37.8 |
| 3.3 | 20.7 | 24.6 | 26.2 | 26.8 | 27.2 | 27.9 | 29.5 | 32.2 | 31.9 | 28.4 | 27.9 | 30.4 |
| 1.1 | 27.8 | 33.1 | 27.6 | 25.9 | 26.5 | 25.8 | 24.6 | 27.3 | 29.6 | 28.6 | 27.7 | 31.0 |
| 0.4 | 45.9 | 48.9 | 39.0 | 39.1 | 38.0 | 38.3 | 37.7 | 38.0 | 45.6 | 44.6 | 39.4 | 42.0 |
| 0.1 | 81.3 | 92.7 | 75.0 | 77.5 | 64.4 | 70.6 | 64.9 | 63.4 | | 76.5 | 81.1 | 77.1 |
| 0.0 | 100.3 | 93.2 | 87.3 | 95.2 | 102.5 | 91.7 | 84.8 | 80.4 | 92.6 | 92.8 | 100.0 | 93.4 |
| 0.0 | 102.1 | 82.7 | 97.9 | 106.3 | 104.0 | 106.0 | 91.7 | 100.5 | 103.3 | 97.0 | 107.5 | 96.5 |
| 0.0 | 97.0 | 101.6 | 111.5 | 107.4 | 108.9 | 106.9 | 97.6 | 107.6 | 107.8 | 96.4 | 102.8 | 97.4 |
| 0.0 | 103.5 | 100.8 | 116.3 | 110.0 | 102.8 | 97.6 | 97.9 | 104.5 | 102.7 | 100.6 | 100.9 | 103.0 |
| 0.0 | 87.6 | 101.8 | 109.5 | 109.7 | 99.2 | 101.2 | 100.2 | 111.5 | 108.9 | 97.5 | 103.7 | 101.8 |

| nM | WV-6416 | | WV-6417 | | WV-6418 | | WV-6419 | | WV-6420 | |
|---|---|---|---|---|---|---|---|---|---|---|
| 30.0 | 29.1 | 31.1 | 26.4 | 21.9 | 30.5 | 26.0 | 35.5 | 33.7 | 17.4 | 13.9 |
| 10.0 | 47.5 | 45.3 | 43.7 | 41.2 | 49.4 | 47.4 | 52.9 | 50.9 | 22.9 | 22.5 |
| 3.3 | 31.2 | 36.6 | 31.9 | 30.2 | 38.5 | 35.3 | 43.3 | 37.7 | 23.4 | 22.6 |
| 1.1 | 33.9 | 38.4 | 30.7 | 31.2 | 38.9 | 31.0 | 36.7 | 34.8 | 31.9 | 26.5 |
| 0.4 | 54.3 | 54.1 | 44.4 | 45.0 | 52.9 | 42.4 | 48.1 | 48.2 | 45.8 | 44.5 |
| 0.1 | 83.0 | 92.1 | 77.1 | 79.1 | 93.2 | 71.6 | 74.7 | 75.0 | 75.3 | 79.0 |
| 0.0 | 97.4 | 101.2 | 102.0 | 99.5 | 102.4 | 87.7 | 96.5 | 92.9 | 94.7 | 107.4 |
| 0.0 | 107.5 | 108.4 | 109.9 | 98.7 | 111.2 | 106.5 | 106.9 | 96.9 | 96.1 | 95.9 |
| 0.0 | 103.9 | 108.7 | 110.8 | 106.0 | 118.1 | 85.0 | 99.8 | 113.2 | 93.6 | 102.2 |
| 0.0 | 102.5 | 108.3 | 109.5 | 107.2 | 114.1 | 104.7 | 104.9 | 99.3 | 101.0 | 113.9 |
| 0.0 | 108.4 | 103.5 | 111.6 | 98.3 | 102.6 | 96.7 | 104.3 | 104.2 | 101.3 | 108.5 |
| nM | WV-6421 | | WV-6422 | | WV-6423 | | WV-6424 | | WV-6425 | |
| 30.0 | 24.1 | 17.4 | 17.5 | 11.4 | 18.6 | 13.3 | 18.1 | 10.7 | 32.0 | 14.5 |
| 10.0 | 27.4 | 25.7 | 24.3 | 20.6 | 22.7 | 20.3 | 23.0 | 19.8 | 30.8 | 23.1 |
| 3.3 | 27.8 | 25.1 | 29.2 | 24.9 | 30.7 | 24.0 | 24.7 | 20.2 | 33.8 | 23.7 |
| 1.1 | 34.8 | 33.4 | 39.0 | 34.0 | 44.0 | 43.2 | 29.2 | 31.2 | 44.6 | 32.3 |
| 0.4 | | | 56.7 | 53.3 | 72.3 | 67.6 | 48.8 | 42.2 | 71.5 | 52.6 |
| 0.1 | 76.4 | 73.7 | 88.7 | 82.2 | 95.7 | 85.9 | 86.4 | 65.6 | 102.5 | 79.1 |
| 0.0 | 99.3 | 85.6 | 102.7 | 93.7 | 99.9 | 89.2 | 97.5 | 81.0 | 117.7 | 96.3 |
| 0.0 | 112.1 | 92.3 | 109.1 | 94.9 | 101.5 | 100.4 | 106.3 | 94.7 | 115.4 | 96.0 |
| 0.0 | 102.1 | 110.0 | 111.0 | 112.5 | 107.5 | 101.5 | 111.7 | 96.5 | 105.8 | 101.5 |
| 0.0 | 105.6 | 97.4 | 99.7 | 100.6 | 106.2 | 105.2 | 101.3 | 105.2 | 102.5 | 98.4 |
| 0.0 | 113.5 | 96.3 | 123.0 | 105.0 | 90.2 | 98.5 | 96.4 | 99.1 | 98.2 | 96.1 |
| nM | WV-6426 | | WV-6427 | | WV-6428 | | WV-6429 | | WV-6430 | |
| 30.0 | 10.5 | 10.8 | 9.3 | 8.3 | 12.5 | 12.5 | 13.5 | 12.6 | 14.2 | 15.7 |
| 10.0 | 17.9 | 15.6 | 16.6 | 15.7 | 16.7 | 21.9 | 18.2 | 20.0 | 19.6 | 17.5 |
| 3.3 | 22.7 | 19.1 | 20.2 | 15.7 | 20.0 | 21.3 | 22.9 | 19.1 | 25.0 | 23.2 |
| 1.1 | 31.9 | 26.4 | 26.8 | 23.4 | 33.5 | 26.7 | 34.1 | 27.0 | 33.4 | 30.8 |
| 0.4 | 55.1 | 44.6 | 41.5 | 37.9 | 48.5 | 47.2 | 52.9 | 39.9 | 49.2 | 39.1 |
| 0.1 | 96.5 | 85.7 | 70.7 | 62.0 | 82.6 | 71.3 | 81.1 | 63.1 | 67.9 | 60.3 |
| 0.0 | 104.1 | 93.5 | 81.4 | 85.9 | 91.2 | 91.2 | 88.7 | 81.5 | 95.3 | 80.3 |
| 0.0 | 116.2 | 115.7 | 105.0 | 93.9 | 98.2 | 88.1 | 94.8 | 89.1 | 88.9 | 91.3 |
| 0.0 | 120.4 | 97.5 | 109.9 | 97.6 | 101.5 | 98.3 | 98.2 | 97.4 | 100.2 | 90.8 |
| 0.0 | 109.0 | 94.1 | 106.2 | 100.8 | 102.8 | 97.6 | 104.9 | 97.8 | 96.8 | 91.6 |
| 0.0 | 109.3 | 98.0 | 108.5 | 92.8 | 112.4 | 108.9 | 99.4 | 96.0 | 96.0 | 99.7 |

| | | | |
|---|---|---|---|
| nM | WV-6764 | WV-6765 | WV-2477 |

| | | | | | | |
|---|---|---|---|---|---|---|
| 30.0 | 16.2 | 20.4 | 15.8 | 12.5 | 96.7 | 84.3 |
| 10.0 | 29.7 | 32.1 | 24.5 | 25.0 | 97.4 | 101.1 |
| 3.3 | 31.2 | 31.9 | 33.1 | 28.8 | 100.1 | 103.7 |
| 1.1 | 46.9 | 36.7 | 43.6 | 42.9 | 112.0 | 104.5 |
| 0.4 | 79.7 | 65.9 | 77.0 | 68.1 | 115.1 | 110.4 |
| 0.1 | 93.1 | 90.4 | 90.1 | 87.7 | 110.1 | 103.6 |
| 0.0 | 100.2 | 99.0 | 108.1 | 105.5 | 98.5 | 105.1 |
| 0.0 | 107.7 | 98.1 | 110.5 | 96.4 | 106.7 | 108.2 |
| 0.0 | 105.2 | 98.4 | 96.8 | 105.4 | 101.7 | 103.6 |
| 0.0 | 98.1 | 99.0 | 88.3 | 97.9 | 95.4 | 101.5 |
| 0.0 | 96.0 | 92.9 | 91.8 | 98.7 | 93.1 | 99.9 |

Table 40. Table 40 shows the in vivo potency for different single-stranded RNAi agents, which target APOC3: WV-4161, WV-3122, WV-6766, and WV-3247. Oligonucleotides were prepared in a LNP (lipid nanoparticle).

TABLE 40
Activity of APOC3 oligonucleotides.

| PBS | LNP-4161 | LNP-3122 | LNP-6766 | LNP-3247 |
|---|---|---|---|---|
| 59.44 | 102.24 | 31.05 | 30.68 | 9.78 |
| 149.41 | 79.13 | 150.39 | 27.9 | 7.44 |
| 128.09 | 46.99 | 52.1 | 17.64 | 45.69 |
| 109.48 | 71.57 | 97.15 | 56.76 | 15.48 |
| 53.59 | 55.19 | 33.26 | 18.76 | 21.88 |

Numbers indicate % APOC3 mRNA remaining (hAPOC3/mHPRT), wherein 59.44 in the first column, second row, indicates 59.44% mRNA remaining. Animals were treated 2×10mpk. Oligonucleotides comprise, at the 5′ end, a OH (WV-4161), 5′-phosphate (PO) (WV-3122), or 5′-vinyl phosphonate (WV-6766 and WV-3247). Various oligonucleotides comprise different patterns of PO or PS in the post-seed region, and a 2′-deoxy T at the penultimate nucleotide.

Table 41. Table 41 shows the in vivo potency for different LNP formulated single-stranded RNAi agents, which target APOC3: WV-4161, WV-3122, WV-6766, and WV-3247.

### Dose: IV 2×10 mpk on Day 1 and Day 4.

TABLE 41
Activity of APOC3 oligonucleotides.

| Day | PBS | | | | | | | | | |
|---|---|---|---|---|---|---|---|---|---|---|
| 1 | 12.92 | 14.05 | 10 | 9.94 | 7.03 | 6.87 | 6.95 | 7.08 | 9.71 | 12 |
| 8 | 11.5 | 11.57 | 7.43 | 7.54 | 6.57 | 8.28 | 5.21 | 4.69 | 10.45 | 10.44 |
| 15 | 9.53 | 12.67 | 8.22 | 6.62 | 6.9 | 8.81 | 2.29 | 2.29 | 6.95 | 9.15 |
| Day | LNP-4161 | | | | | | | | | |
| 1 | 5.35 | 5.81 | 7.35 | 9.29 | 9.75 | 11.5 | 9.82 | 6.01 | 12.54 | 11.65 |
| 8 | 1.8 | 2.22 | 0.25 | 0.32 | 2.48 | 2.55 | 0.98 | 1.02 | 5.27 | 4.48 |
| 15 | 1.12 | 1.44 | 0.08 | 0.08 | 0.91 | 1.21 | 4.54 | 4.52 | 5.77 | 4.69 |
| Day | LNP-3122 | | | | | | | | | |
| 1 | 4.59 | 4.3 | 7.22 | 7.02 | 6.56 | 8.9 | 8.57 | 9.56 | 8.4 | 12.04 |
| 8 | 2.78 | 2.64 | 3.23 | 3.17 | 2.64 | 2.7 | 5.46 | 4.99 | 5.79 | 5.69 |
| 15 | 0.27 | 0.23 | 3.5 | 2.97 | 0.88 | 0.91 | 3.26 | 3.58 | 0.74 | 0.86 |
| Day | LNP-6766 | | | | | | | | | |
| 1 | 6.75 | 9.24 | 7.22 | 8.83 | 5.47 | 5.19 | 10.39 | 11.88 | 9.07 | 11.14 |
| 8 | 3.98 | 0.14 | 3.14 | 3.05 | 0.48 | 0.5 | 6.15 | 4.91 | 3.96 | 3.07 |
| 15 | 1.62 | 1.89 | 1.43 | 1.48 | 0.63 | 0.59 | 4.17 | 3.42 | 1.6 | 1.62 |
| Day | LNP-3247 | | | | | | | | | |
| 1 | 9.35 | 9.46 | 9.7 | 11.82 | 10.27 | 11.21 | 8.4 | 9.58 | 10.07 | 7.91 |
| 8 | 4.32 | 3.37 | 5.38 | 5.35 | 5.09 | 6.11 | 4.39 | 4.67 | 7.3 | 5.85 |
| 15 | 0.88 | 0.85 | 0.43 | 0.48 | 1.11 | 1.17 | 0.34 | 0.38 | 3.75 | 3.22 |

Serum hApoC3 Protein.

Table 42. Table 42 shows the in vivo potency, measured by changes in triglyceride levels, for different different LNP formulated single-stranded RNAi agents, which target APOC3: WV-4161, WV-3122, WV-6766, and WV-3247.

TABLE 42
Activity of APOC3 oligonucleotides.

| | PBS | | | | | LNP-4161 | | | | |
|---|---|---|---|---|---|---|---|---|---|---|
| 1 | 150.15 | 103.35 | 93.6 | 100.1 | 114.4 | 39 | 78 | 134.55 | 59.8 | 160.55 |
| 8 | 18.85 | 95.55 | 81.9 | 76.05 | 89.05 | 22.1 | −2.6 | 78 | 3.25 | 82.55 |
| 15 | 124.15 | 78 | 195.65 | 37.05 | 118.3 | 11.05 | −8.45 | 48.1 | 34.45 | 40.95 |

| | LNP-3122 | | | | | LNP-6766 | | | | |
|---|---|---|---|---|---|---|---|---|---|---|
| 1 | 53.95 | 74.1 | 104.65 | 83.85 | 81.9 | 118.3 | 78.65 | 42.25 | 130.65 | 94.9 |
| 8 | 29.25 | 49.4 | 71.5 | 56.55 | 63.7 | 98.8 | 34.45 | −1.3 | 74.1 | 68.9 |
| 15 | 17.55 | 45.5 | −1.95 | 30.55 | 3.9 | 92.3 | 22.1 | −1.3 | 61.1 | 37.7 |

| | LNP-3247 | | | | |
|---|---|---|---|---|---|
| 1 | 102.05 | 68.9 | 48.1 | 124.15 | 39 |
| 8 | 53.3 | 79.3 | 58.5 | 133.25 | 66.95 |
| 15 | 13.65 | −6.5 | 1.3 | 28.6 | 94.9 |

### Serum Triglycerides.

Additional experiments demonstrated the in vitro activity of different single-stranded RNAi agents, which target APOC3. In one experiment, oligonucleotides WV-5291, WV-6764, and WV-6765 all demonstrated substantial and substantially identical activity in vitro (data not shown). In another experiment, oligonucleotides WV-5290, WV-6431, and WV-6763 all demonstrated substantial and substantially identical activity in vitro (data not shown).

Table 45. Table 45 shows the stability after incubation in rat liver homogenate (24 h) for different single-stranded RNAi agents, which target APOC3: WV-2817, WV-5288, WV-5290, WV-6763, WV-6431, WV-3242, WV-5289, WV-5291, WV-6765, and WV-6764; and Table 45B, WV-1307, WV-1308. The various oligonucleotides differ in length and stereochemistry; in some oligonucleotides, several internucleotidic linkages are stereorandom phosphorothioates; in various oligonucleotides, stereorandom phosphorothioates and/or phosphodiesters are replaced by a phosphorothioate in the Sp configuration.

TABLE 45
Stability of APOC3 oligonucleotides.

| | Percentage of Full-Length |
|---|---|
| | Oligonucleotide at 24 Hr In Rat Liver |
| Oligonucleotide | Homogenate (approximate numbers) |
| WV-2817 | 4 |
| WV-5288 | 11 |
| WV-5290 | 16 |
| WV-6763 | 43 |
| WV-6431 | 73 |
| WV-3242 | 3 |
| WV-5289 | 8 |
| WV-5291 | 9 |
| WV-6765 | 26 |
| WV-6764 | 61 |

Numbers are approximate (2%).

In one experiment, oligonucleotides WV-1307 and 1308 were both tested for their ability to knockdown APOC3 mRNA levels in vitro; both were capable of mediating knockdown of APOC3 mRNA levels in vitro. WV-1307 and WV-1308 differ in their pattern of 2′-F and 2′-OMe modifications.

Table 46. Tables 46A to 46N shows the in vitro potency of various oligonucleotides.

Table 46A shows the in vitro potency of various oligonucleotides in a single-stranded RNAi agent assay; oligonucleotides were derived from strong ASOs, medium ASOs, weak ASOs, dsRNAi agents, or ssRNA.

TABLE 46A
Activity of oligonucleotides.

ssRNAs

| derived | IC50 of APOC3 ssRNAi agents | | | | | | | | | | | |
|---|---|---|---|---|---|---|---|---|---|---|---|---|
| from: | chemistry #2 | | | | chemistry #4 | | | | chemistry #6 | | | |
| strong ASO | 25 | 15 | 6.2 | 4 | 4 | 6.2 | 1.3 | 1.6 | 15 | 15.5 | 15.9 | 1.6 |
| medium ASO | 6.2 | 6.8 | 4 | 15 | 1.6 | 1.2 | 4 | 15 | 1.6 | 4 | 4.8 | 15 |
| weak ASO | 100 | 102 | 25 | 103 | 110 | 15 | 12 | 95 | 120 | 95 | 15 | 110 |
| dsRNA | 100 | | | | 101 | | | | 102 | | | |
| ssRNA | 25 | 100 | | | 1.6 | 30 | | | 15 | 20 | | |

Numbers represent IC50 (nM) of various APOC3 ssRNAi agents derived from strong, medium and weak ASOs (generally operating from a RNaseH mechanism), or derived from dsRNA or ssRNA (generally operating from a RISC-mediated mechanism).
Chemistry #2 has a 5′ PO and a pattern of 2′ modifications of fmfmfmfmfmfmfmfmfmfmm, wherein f is 2′-F and m is 2′-OMe, and a backbone pattern which is all 0, where 0 is PO.
Chemistry #4 has a 5′ PO and a pattern of 2′ modifications of fmfmfmfmfmfmfmfmfmfmm, wherein f is 2′-F and m is 2′-OMe, and a backbone pattern which is XOXOXOXOXOXOXOXOXOXO, where X is stereorandom PS and O is PO.
Chemistry #6 has a 5′ PO and a pattern of 2′ modifications of fmfmfmfmfmfmfmfmfmfmm, wherein f is 2′-F and m is 2′-OMe, and a backbone pattern which is OOOOOOOOOOOOOXXXXXXX, where X is stereorandom PS and O is PO.
The data show that single-stranded RNAi agents derived from double-stranded RNAi agents were generally not efficacious, and generally had lower activity than ssRNAi agents derived from strong ASOs which mediate knockdown via a RNaseH-mediated mechanism (e.g., WV-1275 and WV-1277).

Various ssRNAi agents to APOC3 were constructed which had various 5′ ends.

These 5′ ends include:

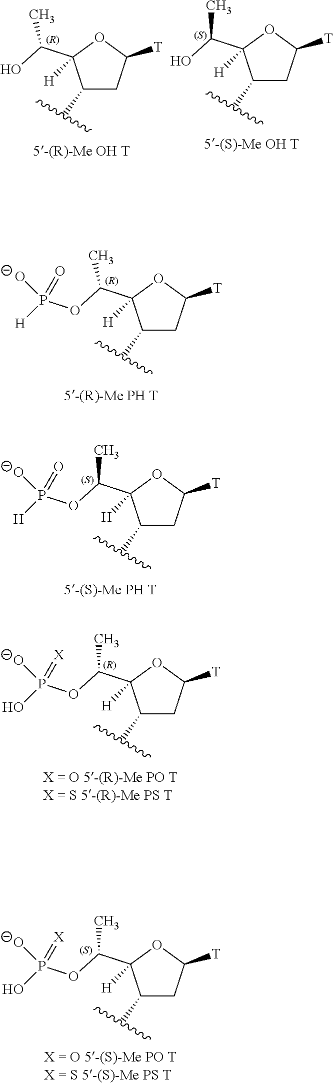

The term 5′-Me generically includes 5′-(S)-Me [or 5MS or S(c)] and 5′-(R)-Me [or 5MR or R(c)]. 5MSdT is the same as 5′-(S)-Me OH T. PO5MSdT is the same as 5′-(S)-Me PO T. PH5MSdT is the same as 5′-(S)-Me PH T. PS5MSdT is the same as 5′-(S)-Me PS T. 5MRdT is the same as 5′-(R)-Me OH T. PO5MRdT is the same as 5′-(R)-Me PO T. PH5MRdT is the same as 5′-(R)-Me PH T. PS5MRdT is the same as 5′-(R)-Me PS T. 5-Me indicates a moiety which is 5′-(R)-Me, 5′-(S)-Me or 5′-Me which is stereorandom.

Tested oligonucleotides having any of these various 5′ ends include: WV-7635, WV-7637, WV-7639, WV-7641, WV-7643, WV-7645, WV-7647, WV-7649, WV-6439, WV-7636, WV-7638, WV-7640, WV-7642, WV-7542, WV-7644, WV-7646, WV-7648, WV-7650, and WV-7542; detailed descriptions of these oligonucleotides can be found in Table 1A.

Activity of these various ssRNAi agents is shown in Tables 2A, 2B, 2C and 2D.

Various hybrid oligonucleotides were constructed. Without wishing to be bound by any particular theory, the present disclosure suggests that, in at least some cases, a hybrid oligonucleotide comprises a structure suitable for an APOC3 oligonucleotide which operates via a RNaseH-mediated mechanism, e.g., a contiguous stretch of nucleotides which are 2′-deoxy, and also a structure suitable for an APOC3 oligonucleotide which operates via a RISC-mediated mechanism, such as a seed region; and the present disclosure suggests that, in at least some cases, a hybrid oligonucleotide is capable of knocking down gene expression via a RNaseH-mediated and/or a RISC-mediated mechanism. Hybrid oligonucleotides constructed include: WV-7523, WV-7525, WV-7527, WV-7524, WV-7526, and WV-7528. These oligonucleotides were tested in vitro for ability to knockdown gene expression in comparison with oligonucleotides including: WV-7672, WV-7521, WV-6763, WV-6431, WV-6439, WV-7673, WV-7522, WV-6765, WV-6764, and WV-6439. The results are shown in the Tables 46H and 461, below.

TABLE 46H
Activity of Oligonucleotides.

| Conc (exp | | | | | | | | |
|---|---|---|---|---|---|---|---|---|
| 10) (nM) | WV-7672 | | WV-7521 | | WV-7523 | | WV-7525 | |
| 1.000 | 0.243 | 0.150 | 0.231 | 0.230 | 0.214 | 0.208 | 0.267 | 0.227 |
| 0.523 | 0.204 | 0.173 | 0.315 | 0.254 | 0.293 | 0.278 | 0.336 | 0.227 |
| 0.046 | 0.273 | 0.237 | 0.495 | 0.382 | 0.511 | 0.477 | 0.497 | 0.383 |
| −0.431 | 0.580 | 0.347 | 0.769 | 0.656 | 0.773 | 0.629 | 0.583 | 0.539 |
| −0.908 | 0.782 | 0.790 | 1.054 | 0.926 | 1.305 | 1.151 | 0.908 | 0.852 |
| −1.386 | 1.042 | 1.093 | 0.925 | 1.451 | 1.258 | 1.191 | 1.100 | 1.057 |
| −1.863 | 1.141 | 1.144 | 1.057 | 1.096 | 1.046 | 1.382 | 1.346 | 1.172 |
| −2.340 | 1.040 | 1.120 | 1.025 | 1.405 | 1.119 | 1.218 | 1.208 | 1.305 |
| −2.817 | 1.023 | 0.937 | 1.022 | 1.028 | 0.882 | 1.114 | 1.080 | 1.071 |
| −3.294 | 1.045 | 1.266 | 1.062 | 1.199 | 1.050 | 1.187 | 0.980 | 1.072 |
| −3.771 | 1.072 | 0.984 | 0.928 | 1.026 | 1.023 | 1.005 | 0.941 | 1.079 |

| Conc (exp | | | | | | | | |
|---|---|---|---|---|---|---|---|---|
| 10) (nM) | WV-7527 | | WV-6763 | | WV-6431 | | WV-6439 (Control) | |
| 1.000 | 0.379 | 0.373 | 0.225 | 0.250 | 0.288 | 0.255 | 0.224 | 0.209 |
| 0.523 | 0.500 | 0.428 | 0.269 | 0.218 | 0.209 | 0.207 | 0.207 | 0.148 |
| 0.046 | 0.736 | 0.617 | 0.221 | 0.164 | 0.224 | 0.183 | 0.226 | 0.217 |
| −0.431 | 1.018 | 0.958 | 0.247 | 0.178 | 0.269 | 0.224 | 0.504 | 0.402 |
| −0.908 | 0.938 | 1.231 | 0.351 | 0.269 | 0.472 | 0.406 | 0.642 | 0.653 |
| −1.386 | 1.231 | 1.326 | 0.531 | 0.489 | 0.719 | 0.768 | 0.937 | 0.959 |
| −1.863 | 1.208 | 1.383 | 0.790 | 0.649 | 0.838 | 0.961 | 1.148 | 1.205 |
| −2.340 | 1.136 | 1.162 | 0.873 | 0.881 | 1.321 | 1.136 | 1.127 | 1.035 |
| −2.817 | 1.106 | 1.084 | 1.080 | 0.962 | 1.119 | 1.311 | 1.141 | 1.035 |
| −3.294 | 0.961 | 1.132 | 1.124 | 1.101 | 0.985 | 1.270 | 0.984 | 0.972 |
| −3.771 | 1.073 | 1.203 | 1.164 | 0.936 | 1.059 | 1.309 | 0.968 | 0.871 |

TABLE 46I
Activity of oligonucleotides.

| Conc (exp | | | | | | | | |
|---|---|---|---|---|---|---|---|---|
| 10) (nM) | WV-7673 | | WV-7522 | | WV-7524 | | WV-7526 | |
| 1.000 | 0.126 | 0.091 | 0.276 | 0.194 | 0.206 | 0.176 | 0.268 | 0.173 |
| 0.523 | 0.191 | 0.105 | 0.352 | 0.256 | 0.383 | 0.315 | 0.381 | 0.250 |
| 0.046 | 0.283 | 0.193 | 0.620 | 0.435 | 0.643 | 0.505 | 0.588 | 0.474 |
| −0.431 | 0.484 | 0.382 | 0.846 | 0.608 | 0.910 | 0.558 | 0.648 | 0.626 |
| −0.908 | 0.804 | 0.717 | 1.041 | 1.143 | 1.085 | 0.992 | 1.154 | 0.951 |
| −1.386 | 1.169 | 0.965 | 1.196 | 1.176 | 1.169 | 1.074 | 1.199 | 1.263 |
| −1.863 | 1.136 | 1.120 | 1.217 | 1.116 | 1.173 | 1.336 | 1.242 | 1.312 |
| −2.340 | 1.136 | 1.475 | 1.224 | 1.565 | 1.140 | 1.716 | 1.162 | 1.095 |
| −2.817 | 0.901 | 0.962 | 1.060 | 1.074 | 1.151 | 1.120 | 1.136 | 1.104 |
| −3.294 | 1.023 | 1.255 | 1.035 | 1.367 | 1.137 | 1.228 | 1.148 | 1.036 |
| −3.771 | 1.022 | 1.017 | 0.925 | 0.984 | 1.094 | 0.963 | 0.993 | 0.947 |

| Conc (exp | | | | | | | | |
|---|---|---|---|---|---|---|---|---|
| 10) (nM) | WV-7528 | | WV-6765 | | WV-6764 | | WV-6439 (Control) | |
| 1.000 | 0.349 | 0.281 | 0.156 | 0.086 | 0.088 | 0.061 | 0.220 | 0.194 |
| 0.523 | 0.473 | 0.372 | 0.220 | 0.143 | 0.186 | 0.119 | 0.225 | 0.149 |
| 0.046 | 0.697 | 0.550 | 0.303 | 0.143 | 0.304 | 0.168 | 0.276 | 0.193 |
| −0.431 | 0.862 | 0.836 | 0.488 | 0.396 | 0.400 | 0.293 | 0.440 | 0.310 |
| −0.908 | 1.083 | 1.087 | 0.776 | 0.566 | 0.812 | 0.601 | 0.735 | 0.585 |
| −1.386 | 1.401 | 1.156 | 1.197 | 0.851 | 1.100 | 0.911 | 0.990 | 0.876 |
| −1.863 | 1.262 | 1.242 | 1.121 | 1.196 | 1.233 | 1.129 | 1.211 | 1.047 |
| −2.340 | 1.786 | 1.213 | 1.221 | 1.027 | 1.301 | 1.303 | 1.226 | 1.082 |
| −2.817 | 1.183 | 1.042 | 1.059 | 1.132 | 1.220 | 1.010 | 1.191 | 1.021 |
| −3.294 | 1.073 | 1.190 | 1.024 | 1.315 | 1.048 | 1.247 | 1.123 | 1.066 |
| −3.771 | 0.943 | 1.065 | 1.094 | 1.033 | 1.146 | 1.042 | 0.968 | 0.880 |

Tables 46J and 46K show in vitro efficacy of various oligonucleotides in Hep3B cells. Various oligonucleotides comprise stereorandom phosphorothioates or sterecontrolled phosphorothioates.

TABLE 46J
Activity of oligonucleotides.

| Conc (exp | | | | | | | | |
|---|---|---|---|---|---|---|---|---|
| 10) (nM) | WV-7672 | | WV-7521 | | WV-7523 | | WV-7525 | |
| 1.000 | 0.243 | 0.150 | 0.231 | 0.230 | 0.214 | 0.208 | 0.267 | 0.227 |
| 0.523 | 0.204 | 0.173 | 0.315 | 0.254 | 0.293 | 0.278 | 0.336 | 0.227 |
| 0.046 | 0.273 | 0.237 | 0.495 | 0.382 | 0.511 | 0.477 | 0.497 | 0.383 |
| −0.431 | 0.580 | 0.347 | 0.769 | 0.656 | 0.773 | 0.629 | 0.583 | 0.539 |
| −0.908 | 0.782 | 0.790 | 1.054 | 0.926 | 1.305 | 1.151 | 0.908 | 0.852 |
| −1.386 | 1.042 | 1.093 | 0.925 | 1.451 | 1.258 | 1.191 | 1.100 | 1.057 |
| −1.863 | 1.141 | 1.144 | 1.057 | 1.096 | 1.046 | 1.382 | 1.346 | 1.172 |
| −2.340 | 1.040 | 1.120 | 1.025 | 1.405 | 1.119 | 1.218 | 1.208 | 1.305 |
| −2.817 | 1.023 | 0.937 | 1.022 | 1.028 | 0.882 | 1.114 | 1.080 | 1.071 |
| −3.294 | 1.045 | 1.266 | 1.062 | 1.199 | 1.050 | 1.187 | 0.980 | 1.072 |
| −3.771 | 1.072 | 0.984 | 0.928 | 1.026 | 1.023 | 1.005 | 0.941 | 1.079 |

| Conc (exp | | | | | | | | |
|---|---|---|---|---|---|---|---|---|
| 10) (nM) | WV-7527 | | WV-6763 | | WV-6431 | | WV-6439 (Control) | |
| 1.000 | 0.379 | 0.373 | 0.225 | 0.250 | 0.288 | 0.255 | 0.224 | 0.209 |
| 0.523 | 0.500 | 0.428 | 0.269 | 0.218 | 0.209 | 0.207 | 0.207 | 0.148 |
| 0.046 | 0.736 | 0.617 | 0.221 | 0.164 | 0.224 | 0.183 | 0.226 | 0.217 |
| −0.431 | 1.018 | 0.958 | 0.247 | 0.178 | 0.269 | 0.224 | 0.504 | 0.402 |
| −0.908 | 0.938 | 1.231 | 0.351 | 0.269 | 0.472 | 0.406 | 0.642 | 0.653 |
| −1.386 | 1.231 | 1.326 | 0.531 | 0.489 | 0.719 | 0.768 | 0.937 | 0.959 |
| −1.863 | 1.208 | 1.383 | 0.790 | 0.649 | 0.838 | 0.961 | 1.148 | 1.205 |
| −2.340 | 1.136 | 1.162 | 0.873 | 0.881 | 1.321 | 1.136 | 1.127 | 1.035 |
| −2.817 | 1.106 | 1.084 | 1.080 | 0.962 | 1.119 | 1.311 | 1.141 | 1.035 |
| −3.294 | 0.961 | 1.132 | 1.124 | 1.101 | 0.985 | 1.270 | 0.984 | 0.972 |
| −3.771 | 1.073 | 1.203 | 1.164 | 0.936 | 1.059 | 1.309 | 0.968 | 0.871 |

TABLE 46K
Activity of oligonucleotides.

| Conc (exp | | | | | | | | |
|---|---|---|---|---|---|---|---|---|
| 10) (nM) | WV-7673 | | WV-7522 | | WV-7524 | | WV-7526 | |
| 1.000 | 0.126 | 0.091 | 0.276 | 0.194 | 0.206 | 0.176 | 0.268 | 0.173 |
| 0.523 | 0.191 | 0.105 | 0.352 | 0.256 | 0.383 | 0.315 | 0.381 | 0.250 |
| 0.046 | 0.283 | 0.193 | 0.620 | 0.435 | 0.643 | 0.505 | 0.588 | 0.474 |
| −0.431 | 0.484 | 0.382 | 0.846 | 0.608 | 0.910 | 0.558 | 0.648 | 0.626 |
| −0.908 | 0.804 | 0.717 | 1.041 | 1.143 | 1.085 | 0.992 | 1.154 | 0.951 |
| −1.386 | 1.169 | 0.965 | 1.196 | 1.176 | 1.169 | 1.074 | 1.199 | 1.263 |
| −1.863 | 1.136 | 1.120 | 1.217 | 1.116 | 1.173 | 1.336 | 1.242 | 1.312 |
| −2.340 | 1.136 | 1.475 | 1.224 | 1.565 | 1.140 | 1.716 | 1.162 | 1.095 |
| −2.817 | 0.901 | 0.962 | 1.060 | 1.074 | 1.151 | 1.120 | 1.136 | 1.104 |
| −3.294 | 1.023 | 1.255 | 1.035 | 1.367 | 1.137 | 1.228 | 1.148 | 1.036 |
| −3.771 | 1.022 | 1.017 | 0.925 | 0.984 | 1.094 | 0.963 | 0.993 | 0.947 |

| Conc (exp | | | | | | | | |
|---|---|---|---|---|---|---|---|---|
| 10) (nM) | WV-7528 | | WV-6765 | | WV-6764 | | WV-6439 (Control) | |
| 1.000 | 0.349 | 0.281 | 0.156 | 0.086 | 0.088 | 0.061 | 0.220 | 0.194 |
| 0.523 | 0.473 | 0.372 | 0.220 | 0.143 | 0.186 | 0.119 | 0.225 | 0.149 |
| 0.046 | 0.697 | 0.550 | 0.303 | 0.143 | 0.304 | 0.168 | 0.276 | 0.193 |
| −0.431 | 0.862 | 0.836 | 0.488 | 0.396 | 0.400 | 0.293 | 0.440 | 0.310 |
| −0.908 | 1.083 | 1.087 | 0.776 | 0.566 | 0.812 | 0.601 | 0.735 | 0.585 |
| −1.386 | 1.401 | 1.156 | 1.197 | 0.851 | 1.100 | 0.911 | 0.990 | 0.876 |
| −1.863 | 1.262 | 1.242 | 1.121 | 1.196 | 1.233 | 1.129 | 1.211 | 1.047 |
| −2.340 | 1.786 | 1.213 | 1.221 | 1.027 | 1.301 | 1.303 | 1.226 | 1.082 |
| −2.817 | 1.183 | 1.042 | 1.059 | 1.132 | 1.220 | 1.010 | 1.191 | 1.021 |
| −3.294 | 1.073 | 1.190 | 1.024 | 1.315 | 1.048 | 1.247 | 1.123 | 1.066 |
| −3.771 | 0.943 | 1.065 | 1.094 | 1.033 | 1.146 | 1.042 | 0.968 | 0.880 |

Tables 46M and 46N show in vitro efficacy of various oligonucleotides in Hep3B cells. Tested oligonucleotides were: WV-6439, WV-7540, WV-7541, WV-7542, WV-7543, and WV-7544. Various oligonucleotides comprise a seed region which has a pattern of 2′-modifications of alternating 2′-F and 2′-OMe; a pattern with an initial 2′-F followed by 2′-OME; or a pattern of all 2′-OMe.

TABLE 46M
Activity of oligonucleotides.

| | | | |
|---|---|---|---|
| Conc (exp | | | |
| 10) (nM) | WV-6439 | WV-7540 | WV-7541 |

| | | | | | | |
|---|---|---|---|---|---|---|
| 1 | 0.173 | 0.264 | 0.144 | 0.254 | 0.266 | 0.352 |
| 0.523 | 0.124 | 0.169 | 0.192 | 0.235 | 0.42 | 0.558 |
| 0.046 | 0.171 | 0.234 | 0.245 | 0.37 | 0.613 | 0.785 |
| −0.431 | 0.379 | 0.346 | 0.436 | 0.488 | 0.785 | 0.997 |
| −0.908 | 0.658 | 0.507 | 0.772 | 0.943 | 0.941 | 1.233 |
| −1.386 | 0.791 | 0.823 | 1.196 | 0.982 | 1.119 | 1.043 |
| −1.863 | 0.901 | 0.903 | 1.053 | 1.073 | 1.099 | 1.165 |
| −2.34 | 0.956 | 0.949 | 1.031 | 1.197 | 0.975 | 1.296 |
| −2.817 | 1.006 | 0.983 | 1.099 | 1.123 | 1.112 | 0.966 |
| −3.294 | 1.034 | 0.959 | 1.014 | 0.969 | 0.994 | 1.045 |

TABLE 46N
Activity of oligonucleotides.

| Conc | | | | | | |
|---|---|---|---|---|---|---|
| (exp | | | | | | |
| 10) | | | | | | |
| (nM) | WV-7542 | | WV-7543 | | WV-7544 | |
| 1 | 0.043 | 0.104 | 0.090 | 0.150 | 0.128 | 0.188 |
| 0.523 | 0.082 | 0.156 | 0.143 | 0.249 | 0.236 | 0.417 |
| 0.046 | 0.168 | 0.257 | 0.223 | 0.365 | 0.402 | 0.756 |
| −0.431 | 0.367 | 0.462 | 0.368 | 0.797 | 0.697 | 1.024 |
| −0.908 | 0.552 | 0.854 | 0.601 | 0.968 | 0.858 | 1.094 |
| −1.386 | 0.959 | 1.046 | 0.879 | 1.327 | 0.975 | 1.121 |
| −1.863 | 0.919 | 1.233 | 1.021 | 1.341 | 1.003 | 1.101 |
| −2.34 | 1.157 | | 1.185 | 1.150 | 1.054 | 1.351 |
| −2.817 | 1.064 | 1.297 | 1.136 | 1.064 | 1.104 | 0.956 |
| −3.294 | 1.013 | 0.993 | 0.975 | 0.946 | 0.914 | 0.919 |

As shown in Table 47, additional single-stranded RNAi agents to APOC3 were tested.

TABLE 47A
Activity of APOC3 oligonucleotides.

| Control | | WV-1868 | | WV-2110 | | WV-2388 | | WV-2111 | | WV-2389 | |
|---|---|---|---|---|---|---|---|---|---|---|---|
| 0.99 | 1.02 | 0.52 | 0.57 | 0.33 | 0.39 | 0.43 | 0.49 | 0.55 | 0.43 | 0.59 | 0.62 |

Numbers represent relative residual APOC3 mRNA level after oligonucleotide treatment (400 pM). The number 1 would represent 100% APOC3 mRNA level (relative to control); 0 would represent 0% APOC3 mRNA level or 100% knockdown. WV-2111 and WV-2389 have a hybrid format.

TABLE 47B
Activity of APOC3 oligonucleotides.

| Conc.(nM) | | | | | | |
|---|---|---|---|---|---|---|
| (exp 10) | WV-2113 | | WV-2148 | | WV-2149 | |
| 1.398 | 0.03 | 0.02 | 0.01 | 0.01 | 0.02 | 0.02 |
| 0.792 | 0.15 | 0.14 | 0.04 | 0.02 | 0.07 | 0.04 |
| 0.204 | 0.44 | 0.46 | 0.28 | 0.22 | 0.34 | 0.30 |
| −0.398 | 0.71 | 0.75 | 0.54 | 0.51 | 0.71 | 0.53 |
| −1.000 | 0.87 | 0.88 | 0.78 | 0.63 | 0.75 | 0.58 |
| −1.602 | 0.82 | 0.99 | 0.67 | 1.09 | 0.79 | 0.93 |
| −2.204 | 0.92 | 1.13 | 0.83 | 0.71 | 0.83 | 0.76 |
| −2.824 | 0.99 | 1.22 | 1.02 | 0.87 | 0.89 | 0.78 |

TABLE 47C
IC50 of various APOC3 oligonucleotides.

| Oligonucleotide | IC 50 (pM) |
|---|---|
| WV-1308 | 102 |
| WV-1308 | 144 |
| WV-1868 | 266 |
| WV-2110 | 64 |
| WV-2110 | 64 |
| WV-2111 | 163 |
| WV-2111 | 163 |
| WV-2114 | 705 |
| WV-2114 | 628 |
| WV-2150 | 70 |
| WV-2151 | 87 |
| WV-2152 | 427 |
| WV-2153 | 371 |
| WV-2154 | 120 |
| WV-2155 | 74 |
| WV-2156 | 118 |
| WV-2157 | 245 |

WV-2114, WV-2152, and WV-2153 have a hybrid format. Oligonucleotides which are repeated with different numbers indicate different replicates or experiments.

TABLE 47D
Activity of APOC3 oligonucleotides.

| | | | |
|---|---|---|---|
| Conc (exp | | | |
| 10) nM | WV-7540 | WV-8427 | WV-8429 |

| | | | | | | |
|---|---|---|---|---|---|---|
| 1.00 | 0.13 | 0.15 | 0.20 | 0.17 | 0.14 | 0.15 |
| 0.52 | 0.16 | 0.25 | 0.22 | 0.26 | 0.15 | 0.19 |
| 0.05 | 0.27 | 0.32 | 0.37 | 0.35 | 0.26 | 0.22 |
| −0.43 | 0.40 | 0.47 | 0.40 | 0.54 | 0.28 | 0.30 |
| −0.91 | 0.56 | 0.64 | 0.63 | 0.71 | 0.45 | 0.49 |
| −1.39 | 0.69 | 0.80 | 0.80 | 0.92 | 0.61 | 0.71 |
| −1.86 | 0.73 | 0.92 | 0.91 | 0.97 | 0.79 | 0.96 |
| −2.34 | 0.69 | 0.84 | 1.00 | 0.98 | 1.01 | 0.95 |
| −2.82 | 0.72 | 0.92 | 1.00 | 1.04 | 0.95 | 1.01 |
| −3.29 | 0.77 | 1.34 | 0.91 | 1.60 | 0.97 | 0.95 |
| −3.77 | 0.64 | 1.05 | 0.88 | 1.25 | 1.31 | 0.96 |

| | | | |
|---|---|---|---|
| 1.00 | WV-8431 | WV-6439 | WV-6439 |

| | | | | | | |
|---|---|---|---|---|---|---|
| 0.52 | 0.17 | 0.17 | 0.21 | 0.21 | 0.19 | 0.19 |
| 0.05 | 0.21 | 0.21 | 0.17 | 0.17 | 0.18 | 0.18 |
| −0.43 | 0.23 | 0.23 | 0.22 | 0.22 | 0.19 | 0.19 |
| −0.91 | 0.29 | 0.29 | 0.23 | 0.23 | 0.27 | 0.27 |
| −1.39 | 0.49 | 0.49 | 0.52 | 0.52 | 0.40 | 0.40 |
| −1.86 | 0.75 | 0.75 | 0.74 | 0.74 | 0.57 | 0.57 |
| −2.34 | 0.90 | 0.90 | 0.91 | 0.91 | 0.70 | 0.70 |
| −2.82 | 1.00 | 1.00 | 0.99 | 0.99 | 0.80 | 0.80 |
| −3.29 | 1.06 | 1.06 | 1.05 | 1.05 | 0.94 | 0.94 |
| −3.77 | 1.12 | 1.12 | 0.95 | 0.95 | 0.98 | 0.98 |

| | | |
|---|---|---|
| 1.00 | WV-6431 | WV-6439 Plate Control |

| | | | | |
|---|---|---|---|---|
| 0.52 | 0.21 | 0.21 | 0.20 | 0.20 |
| 0.05 | 0.28 | 0.28 | 0.21 | 0.21 |
| −0.43 | 0.18 | 0.18 | 0.23 | 0.23 |
| −0.91 | 0.23 | 0.23 | 0.29 | 0.29 |
| −1.39 | 0.41 | 0.41 | 0.54 | 0.54 |
| −1.86 | 0.67 | 0.67 | 0.67 | 0.67 |
| −2.34 | 0.73 | 0.73 | 0.75 | 0.75 |
| −2.82 | 0.85 | 0.85 | 0.85 | 0.85 |
| −3.29 | 1.05 | 1.05 | 0.77 | 0.77 |
| −3.77 | 0.90 | 0.90 | 0.86 | 0.86 |

In this and various other tables wherein the conc. of the oligonucleotide is a negative number, the negative number (e.g.,-3.77) indicates an exponent of 10 (exp10) or log.

TABLE 47E
Activity of APOC3 oligonucleotides.

| Conc (exp | | | |
|---|---|---|---|
| 10) nM | 7543 | 8428 | 8430 |

| | | | | | | |
|---|---|---|---|---|---|---|
| 1.00 | 0.18 | 0.18 | 0.18 | 0.19 | 0.24 | 0.21 |
| 0.52 | 0.24 | 0.25 | 0.20 | 0.23 | 0.33 | 0.33 |
| 0.05 | 0.39 | 0.39 | 0.37 | 0.37 | 0.46 | 0.40 |
| −0.43 | 0.57 | 0.53 | 0.49 | 0.50 | 0.65 | 0.56 |
| −0.91 | 0.68 | 0.69 | 0.76 | 0.78 | 0.84 | 0.66 |
| −1.39 | 0.88 | 0.82 | 0.88 | 0.93 | 0.91 | 0.84 |
| −1.86 | 0.93 | 0.87 | 0.94 | 1.06 | 1.13 | 0.95 |
| −2.34 | 0.94 | 0.83 | 0.99 | 1.13 | 1.18 | 0.93 |
| −2.82 | 0.99 | 0.87 | 1.00 | 1.00 | 1.01 | 0.92 |
| −3.29 | 0.95 | 0.83 | 1.01 | 1.06 | 1.16 | 1.02 |
| −3.77 | 1.11 | 0.80 | 1.00 | 1.02 | 1.03 | 0.99 |

| | | | |
|---|---|---|---|
| Conc (exp | | | |
| 10) | 8432 | 7542 | 6765 |

| | | | | | | |
|---|---|---|---|---|---|---|
| 1.00 | 0.21 | 0.20 | 0.09 | 0.14 | 0.16 | 0.17 |
| 0.52 | 0.24 | 0.25 | 0.14 | 0.15 | 0.21 | 0.21 |
| 0.05 | 0.33 | 0.36 | 0.23 | 0.27 | 0.26 | 0.25 |
| −0.43 | 0.47 | 0.55 | 0.37 | 0.40 | 0.46 | 0.38 |
| −0.91 | 0.73 | 0.71 | 0.59 | 0.63 | 0.66 | 0.66 |
| −1.39 | 0.85 | 0.86 | 0.82 | 0.85 | 0.94 | 0.82 |
| −1.86 | 0.97 | 0.95 | 0.96 | 1.09 | 0.95 | 1.04 |
| −2.34 | 1.08 | 1.06 | 1.13 | 1.53 | 0.98 | 1.02 |
| −2.82 | 0.95 | 1.10 | 1.02 | 0.92 | 0.99 | 1.11 |
| −3.29 | 1.00 | 0.99 | 1.05 | 1.46 | 0.96 | 1.07 |
| −3.77 | 0.97 | 1.08 | 1.01 | 1.20 | 0.91 | 1.02 |

| | | |
|---|---|---|
| Conc (exp | | |
| 10) | 6764 | 6439 Plate Control |

| | | | | |
|---|---|---|---|---|
| 1.00 | 0.12 | 0.13 | 0.22 | 0.20 |
| 0.52 | 0.19 | 0.23 | 0.18 | 0.16 |
| 0.05 | 0.30 | 0.32 | 0.22 | 0.22 |
| −0.43 | 0.41 | 0.47 | 0.33 | 0.41 |
| −0.91 | 0.70 | 0.83 | 0.54 | 0.69 |
| −1.39 | 0.83 | 0.87 | 0.84 | 0.85 |
| −1.86 | 0.90 | 1.08 | 1.02 | 1.04 |
| −2.34 | 1.01 | 1.06 | 0.89 | 1.00 |
| −2.82 | 1.03 | 1.02 | 1.09 | 1.08 |
| −3.29 | 0.98 | 1.46 | 0.99 | 1.17 |
| −3.77 | 1.10 | 1.06 | 1.09 | 1.04 |

TABLE 47F
Activity of APOC3 oligonucleotides.

| | 100 nM | | 25 nM | | 6.2 nM | |
|---|---|---|---|---|---|---|
| WV-1307 | 0.99619 | 0.7816 | 0.51566 | 0.47781 | 0.48785 | 0.46474 |
| WV-1308 | 0.72926 | 0.91035 | 0.32635 | 0.28411 | 0.21984 | 0.21984 |
| WV-1238 | 0.42765 | 0.63927 | 0.25962 | 0.21088 | 0.34978 | 0.25078 |
| WV-1240 | 0.3241 | 0.21531 | 0.1798 | 0.20229 | 0.36463 | 0.26325 |
| WV-1800 | 0.30239 | 0.31306 | 0.28019 | 0.18231 | 0.24392 | 0.31306 |
| WV-1801 | 0.50856 | 0.51925 | 0.73944 | 0.41596 | 0.35466 | 0.2389 |
| WV-1802 | 0.3109 | 0.23237 | 0.54506 | 0.45203 | 0.25962 | 0.22137 |
| WV-1803 | 0.3109 | 0.21531 | 0.30875 | 0.24057 | 0.14809 | 0.13346 |
| WV-1270 | 0.69472 | 0.8553 | 0.44891 | 0.34736 | 0.43663 | 0.45203 |
| WV-1272 | 0.64819 | 0.58015 | 0.40179 | 0.43663 | 0.66181 | 0.6175 |
| WV-1824 | 0.41885 | 0.57614 | 0.37488 | 0.37749 | 0.32862 | 0.54506 |
| WV-1825 | 0.48785 | 0.48785 | 0.40458 | 0.40179 | 0.38542 | 0.40179 |
| WV-1826 | 0.43062 | 0.38542 | 0.24905 | 0.33786 | 0.38011 | 0.36211 |
| WV-1827 | 0.69955 | 0.70932 | 0.37749 | 0.56039 | 0.48113 | 0.4981 |
| WV-1275 | 0.24 | 0.4 | 0.22 | 0.16 | 0.24 | 0.29 |
| WV-1277 | 0.1 | 0.11 | 0.15 | 0.1 | 0.19 | 0.24 |
| WV-1828 | 0.19405 | 0.26692 | 0.16431 | 0.14107 | 0.18874 | 0.17011 |
| WV-1829 | 0.23237 | 0.26325 | 0.2229 | 0.15762 | 0.23237 | 0.20511 |
| WV-1830 | 0.16318 | 0.19271 | 0.20654 | 0.12196 | 0.18874 | 0.16545 |
| WV-1831 | 0.13346 | 0.16431 | 0.12539 | 0.08505 | 0.15982 | 0.15225 |
| WV-1307 | 0.43 | 0.46 | 0.36 | 0.34 | 0.37 | 0.35 |
| WV-1308 | 0.28 | 0.27 | 0.11 | 0.12 | 0.16 | 0.16 |
| D73 | 0.04 | 0.03 | 0.04 | 0.03 | 0.06 | 0.06 |
| D74 | 0.02 | 0.02 | 0.02 | 0.02 | 0.07 | 0.05 |
| WV-1238 | 0.55 | 0.14 | 0.1 | 0.14 | 0.32 | 0.39 |
| WV-1240 | 0.18 | 0.18 | 0.28 | 0.24 | 0.55 | 0.5 |
| WV-1800 | 0.27 | 0.18 | 0.2 | 0.08 | 0.14 | 0.18 |
| WV-1801 | 0.58 | 0.38 | 0.34 | 0.59 | 0.25 | 0.23 |
| WV-1802 | 0.23 | 0.3 | 0.16 | 0.63 | 0.2 | 0.22 |
| WV-1803 | 0.15 | 0.19 | 0.17 | 0.14 | 0.11 | 0.13 |
| WV-1270 | 0.61 | 0.61 | 0.51 | 0.26 | 0.29 | 0.32 |
| WV-1272 | 0.49 | 0.51 | 0.42 | 0.36 | 0.51 | 0.73 |
| WV-1824 | 0.38 | 0.43 | 0.22 | 0.25 | 0.31 | 0.38 |
| WV-1825 | 0.4 | 0.33 | 0.15 | 0.4 | 0.29 | 0.32 |
| WV-1826 | 0.29 | 0.3 | 0.2 | 0.2 | 0.26 | 0.31 |
| WV-1827 | 0.97 | 0.53 | 0.46 | 0.39 | 0.44 | 0.48 |
| WV-1275 | 0.42 | 0.27 | 0.17 | 0.16 | 0.26 | 0.35 |
| WV-1277 | 0.1 | 0.08 | 0.09 | 0.09 | 0.26 | 0.28 |
| WV-1828 | 0.14 | 0.14 | 0.22 | 0.36 | 0.22 | 0.13 |
| WV-1829 | 0.2 | 0.2 | 0.14 | 0.23 | 0.14 | 0.19 |
| WV-1830 | 0.17 | 0.12 | 0.34 | 0.17 | 0.15 | 0.17 |
| WV-1831 | 0.1 | 0.11 | 0.11 | 0.07 | 0.12 | 0.14 |

Oligonucleotides were also tested at 0.4 and 1 nM and 25 and 6.2 pM, at which concentrations, the oligonucleotides showed decreased ability to knock down APOC3 relative to the higher concentrations (data not shown).

TABLE 47G
Activity of APOC3 oligonucleotides.

| Log Conc. (nM) | H2O | | WV-4186 | | WV-4255 | |
|---|---|---|---|---|---|---|
| 25 | 1.13 | 0.87 | 0.03 | 0.03 | 0.04 | 0.04 |
| 6.25 | 1.13 | 0.59 | 0.1 | 0.04 | 0.31 | 0.21 |
| 1.5625 | 0.99 | 0.71 | 0.13 | 0.08 | 0.46 | 0.4 |
| 0.390625 | 0.86 | 0.94 | 0.25 | 0.33 | 0.73 | 0.82 |
| 0.097656 | 0.68 | 0.94 | 0.7 | 0.48 | 0.92 | 1.22 |
| 0.024414 | 0.77 | 0.9 | 0.6 | 0.58 | 0.9 | 1.08 |
| 0.006104 | 0.7 | 1.28 | 0.64 | 1.6 | 0.73 | 1.39 |
| 0.001526 | 0.76 | 0.94 | 0.9 | 0.91 | 0.76 | 1.03 |
| Log Conc. (nM) | WV-4245 | | WV-4260 | | WV-4250 | |
| 25 | 0.08 | 0.07 | | 0.04 | | 0.08 |
| 6.25 | 0.31 | 0.17 | 0.3 | 0.19 | 0.24 | 0.14 |
| 1.5625 | 0.41 | 0.56 | 0.52 | 0.39 | 0.38 | 0.37 |
| 0.390625 | 0.74 | 0.84 | 0.69 | 0.79 | 0.66 | 0.6 |
| 0.097656 | 0.73 | 0.96 | 0.9 | 1 | 0.99 | 0.98 |
| 0.024414 | 0.71 | 0.8 | 0.84 | 1.05 | 0.89 | 0.98 |
| 0.006104 | 0.77 | 1.75 | 0.72 | 1.4 | 0.72 | 1.26 |
| 0.001526 | 0.74 | 1.05 | 1.15 | 1.08 | 0.74 | 1.32 |
| Log Conc. (nM) | WV-4145 | | WV-4178 | | WV-4156 | |
| 25 | | 0.02 | | 0.02 | 0.02 | |
| 6.25 | 0.18 | 0.12 | 0.33 | 0.23 | 0.2 | 0.15 |
| 1.5625 | 0.36 | 0.32 | 0.63 | 0.47 | 0.36 | 0.32 |
| 0.390625 | 0.74 | 0.7 | 0.99 | 0.89 | 0.74 | 0.85 |
| 0.097656 | 0.84 | 1.08 | 0.87 | 1.11 | 0.92 | 0.97 |
| 0.024414 | 0.66 | 1.11 | 0.83 | 0.99 | 0.86 | 1.43 |
| 0.006104 | 0.82 | 1.3 | 0.86 | 1.47 | 0.93 | 1.51 |
| 0.001526 | 0.89 | 1.23 | 0.78 | 1.2 | 0.9 | 1.08 |

| | | |
|---|---|---|
| Log Conc. (nM) | WV-4172 | WV-4149 |

| | | | | |
|---|---|---|---|---|
| 25 | | 0.07 | 0.17 | 0.1 |
| 6.25 | 0.68 | 0.39 | 0.56 | 0.49 |
| 1.5625 | 0.77 | 0.78 | 0.84 | 0.76 |
| 0.390625 | 1.19 | 1.05 | 0.81 | 0.88 |
| 0.097656 | 0.93 | 1.16 | 0.85 | 0.9 |
| 0.024414 | 1.11 | 1.2 | 0.84 | 1.19 |
| 0.006104 | 1.17 | 1.47 | 1.91 | 1.08 |
| 0.001526 | 0.78 | 1.14 | 0.79 | 1.33 |

TABLE 47H
Activity of APOC3 oligonucleotides.

| Log Conc. (nM) | 25 | 6.25 | 1.562 | 0.390 | 0.0976 | 0.0244 | 0.00610 | 0.0015 |
|---|---|---|---|---|---|---|---|---|
| H2O | 0.74 | 0.83 | 1.09 | 0.9 | 1.35 | 1.25 | 0.93 | 1.13 |
| | 1.25 | 0.92 | 0.89 | 1.1 | 0.84 | 0.96 | 1.2 | 0.84 |
| WV-4186 | 0.08 | 0.05 | 0.13 | 0.24 | 0.42 | 0.67 | 0.96 | 1.23 |
| | 0.03 | 0.05 | 0.1 | 0.25 | 0.38 | 0.7 | 1.04 | 0.7 |
| WV-4182 | 0.1 | 0.51 | 0.79 | 1.14 | 1.32 | 1.36 | 1.42 | 1.16 |
| | 0.1 | 0.49 | 0.61 | 0.51 | 0.96 | 0.96 | 1 | 0.86 |
| WV-4160 | 0.12 | 0.55 | 0.82 | 1.19 | 1.22 | 1.39 | 1.18 | 1.44 |
| | 0.19 | 0.52 | 0.71 | 1.04 | 1.11 | 1.15 | 1.02 | 1.08 |
| WV-4256 | 0.2 | 0.77 | 1.17 | 1.5 | 1.3 | 1.5 | 1.34 | 1.63 |
| | 0.19 | 0.67 | 0.84 | 1.02 | 1.15 | 1.6 | 0.99 | 0.87 |
| WV-4246 | 0.18 | 0.98 | 1.47 | 1.44 | 1.48 | 1.38 | 1.23 | 1.38 |
| | 0.18 | 0.55 | 0.71 | 0.51 | 1.06 | 1.21 | 1.1 | 1.18 |
| WV-4261 | 0.13 | 0.84 | 1.05 | 1.19 | 0.96 | 1.82 | 1.62 | 1.61 |
| | 0.15 | 0.55 | 0.79 | 0.51 | 1.1 | 1.2 | 1.08 | 0.96 |
| WV-4251 | 0.09 | 0.72 | 1.06 | 1.25 | 1.31 | 1.44 | 1.51 | 1.63 |
| | 0.1 | 0.37 | 0.75 | 0.51 | 1.08 | 1.41 | 0.98 | 1.32 |
| WV-4257 | 0.13 | 0.77 | 0.95 | 1.56 | 1.54 | 1.19 | 1.69 | 1.8 |
| | 0.21 | 0.64 | 0.86 | 0.51 | 1.02 | 1.32 | 0.9 | 0.83 |
| WV-4247 | 0.05 | 0.46 | 0.78 | 1.23 | 1.32 | 1.55 | 1.3 | 1.43 |
| | 0.06 | 0.39 | 0.66 | 1.13 | 0.97 | 1.28 | 1.04 | 0.94 |
| WV-4262 | 0.13 | 0.62 | 1.07 | 1.34 | 1.05 | 1.3 | 1.36 | 1.65 |
| | 0.19 | 0.58 | 0.9 | 0.51 | 0.87 | 1.11 | 0.89 | 1.04 |
| WV-4252 | 0.04 | 0.3 | 0.5 | 1.02 | 1.02 | 1.11 | 1.04 | 1.22 |
| | 0.06 | 0.26 | 0.59 | 0.84 | 0.87 | 1.05 | 0.76 | 0.8 |

| Conc. (nM) | 25 | 6.25 | 1.56 | 0.3906 | 0.0976 | 0.0244 | 0.00610 | 0.00152 |
|---|---|---|---|---|---|---|---|---|
| H2O | 0.73 | 0.68 | 0.72 | | 1.13 | | 0.75 | 0.91 |
| | | 0.52 | | 1.22 | 0.83 | 0.97 | 0.76 | 0.87 |
| WV-4248 | 0.27 | 0.65 | 0.98 | | 1.03 | 0.87 | 0.91 | 0.98 |
| | 0.18 | 0.78 | | 1.19 | | 0.91 | 1.09 | 1.15 |
| WV-4162 | 0.14 | 0.08 | 0.29 | | 1.03 | 0.76 | 0.93 | 0.88 |
| | 0.07 | 0.08 | 0.29 | 0.83 | 0.87 | 1.14 | 1.09 | 1.04 |
| WV-4139 | 0.08 | 0.08 | 0.19 | | 1.02 | 1 | 1.15 | 1.16 |
| | 0.07 | 0.08 | 0.25 | 0.63 | 0.94 | 1.1 | 1.21 | |
| WV-4173 | 0.08 | 0.08 | 0.21 | | 0.94 | 0.8 | 0.95 | 1.3 |
| | 0.08 | 0.08 | 0.19 | 0.56 | 0.97 | 0.92 | 1.09 | 1.28 |
| WV-4150 | 0.05 | 0.08 | 0.15 | | 0.93 | 1.13 | 1.29 | 1.06 |
| | 0.05 | 0.1 | 0.21 | 0.31 | 0.8 | 1.31 | | |
| WV-4167 | 0.07 | 0.23 | 0.74 | | 0.85 | 1.13 | 1.09 | 1.13 |
| | 0.12 | 0.32 | 0.77 | 0.69 | 1.31 | | 1.19 | 1.41 |
| WV-4144 | 0.06 | 0.14 | 0.27 | | 0.91 | 0.91 | 0.72 | 1.05 |
| | 0.08 | 0.19 | 0.53 | 0.97 | 1.36 | 1.06 | 1.2 | 1.08 |
| WV-3137 | 0.07 | 0.25 | 0.42 | 1.02 | 1.18 | 0.95 | 0.92 | 0.86 |
| | 0.07 | 0.25 | 0.66 | 1.11 | 1.04 | 1.1 | 0.95 | 1.06 |
| WV-4155 | 0.07 | 0.1 | 0.16 | 0.67 | 0.77 | 0.75 | 0.87 | 0.81 |
| | 0.16 | 0.1 | 0.4 | 0.95 | | 1.02 | 0.68 | 1.06 |
| WV-4166 | 0.16 | 0.23 | 0.16 | 0.84 | 0.66 | 0.77 | 0.77 | 0.88 |
| | 0.15 | 0.27 | 0.68 | 0.76 | 1.06 | 0.79 | 0.79 | 0.88 |
| WV-4143 | 0.05 | 0.17 | 0.16 | | 0.7 | 0.74 | 0.86 | 0.97 |
| | 0.05 | 0.2 | 0.47 | 1.09 | 1.05 | 0.69 | 0.55 | 0.85 |

| Log Conc. (nM) | 25 | 6.25 | 1.5625 | 0.3906 | 0.0976 | 0.0244 | 0.0061 | 0.00152 |
|---|---|---|---|---|---|---|---|---|
| H2O | 0.78 | 0.75 | 0.78 | 0.95 | 0.7 | 0.84 | 1.01 | 1.06 |
| | 0.68 | 0.79 | 0.84 | 0.98 | 0.56 | | 0.57 | 0.68 |
| WV-4186 | 0.06 | 0.09 | 0.12 | 0.15 | 0.61 | 0.77 | 0.82 | 0.98 |
| | 0.05 | 0.12 | 0.14 | 0.23 | 0.38 | 0.69 | 1.17 | 0.83 |
| WV-4258 | 0.29 | 0.56 | 0.71 | 0.75 | 0.81 | 0.98 | 1.01 | 1.25 |
| | 0.34 | 0.45 | 0.87 | | 0.88 | 0.82 | | 0.92 |
| WV-4248 | 0.79 | 0.69 | 0.82 | 0.77 | 0.83 | 0.86 | 0.87 | 1.01 |
| | 0.93 | 0.87 | 0.77 | | 0.8 | 1.07 | 0.97 | 1.02 |
| WV-4263 | 0.72 | 0.64 | 0.85 | 0.93 | 0.95 | 1.01 | 1.03 | 0.91 |
| | 0.81 | 0.73 | | 0.9 | | 0.86 | 0.27 | 1 |
| WV-4253 | 0.75 | 0.84 | 0.88 | 0.85 | 0.83 | 0.71 | 0.65 | 0.98 |
| | 0.92 | 0.82 | 0.73 | 0.85 | 1.02 | 0.98 | 1.03 | 1.04 |
| WV-4161 | 0.22 | 0.22 | 0.39 | 0.65 | 1.03 | 0.99 | 0.71 | 1.08 |
| | 0.17 | 0.22 | 0.35 | 0.56 | 0.93 | 0.96 | 0.94 | 1 |
| WV-2817 | 0.2 | 0.23 | 0.34 | 1.26 | 0.79 | 0.82 | 1.05 | 1.12 |
| | 0.19 | 0.24 | 0.33 | 0.64 | 0.68 | | 0.94 | 1 |
| WV-3122 | 0.19 | 0.16 | 0.31 | | 0.79 | 0.78 | 1.08 | 0.96 |
| | 0.11 | 0.18 | 0.36 | 0.44 | 0.54 | 0.81 | 0.82 | 0.88 |
| WV-3021 | 0.28 | | 0.28 | 0.45 | 0.61 | 0.94 | 1.07 | 1.06 |
| | | 0.17 | 0.31 | 0.47 | 0.64 | 0.86 | 0.9 | 1.08 |
| WV-4171 | 0.32 | 0.62 | 0.86 | | 1 | 0.61 | 1.01 | 1.08 |
| | 0.23 | 0.69 | 1.08 | | 1.12 | 1.05 | 0.91 | 0.93 |
| WV-4148 | 0.32 | 0.52 | 0.92 | 0.83 | 1.26 | 0.73 | | 1.01 |
| | 0.22 | 0.59 | 1.16 | 1.1 | 0.86 | 1.12 | | 1.13 |
| H2O | 0.78 | 0.86 | 0.71 | 0.67 | 0.9 | 0.65 | 0.73 | 0.92 |
| | 0.62 | 0.73 | 0.93 | 0.94 | | 0.87 | 0.72 | 0.78 |
| WV-4186 | | 0.07 | 0.18 | 0.2 | 0.32 | 0.68 | 0.69 | 0.7 |
| | 0.21 | 0.11 | 0.12 | 0.24 | 0.36 | 0.61 | 0.87 | 0.89 |
| WV-4181 | 0.24 | 0.85 | 0.75 | 0.74 | 0.96 | 0.81 | 0.82 | 1.05 |
| | 0.18 | 0.46 | 0.9 | 0.9 | | 0.76 | 1.09 | 0.85 |
| WV-4159 | 0.26 | 0.45 | | 0.75 | 0.8 | 0.98 | 0.91 | 0.92 |
| | 0.17 | 0.27 | | 0.76 | 0.75 | 0.87 | 0.92 | 0.98 |
| WV-4170 | 0.38 | 0.96 | 0.73 | 0.88 | 0.35 | 1.08 | 1.15 | 1.1 |
| | 0.3 | 0.6 | 0.88 | 0.94 | 0.85 | 1.09 | 0.84 | 1 |
| WV-4147 | 0.22 | 0.46 | 0.61 | 0.19 | 0.81 | 1.12 | 0.95 | 1.12 |
| | 0.2 | 0.34 | | | 0.8 | 0.58 | 0.76 | 1.03 |
| WV-4180 | 0.43 | 0.77 | 0.29 | 0.44 | 1 | 1.01 | 1.21 | 1.36 |
| | 0.41 | 0.46 | 0.65 | 0.32 | 0.91 | 0.44 | 1.01 | 0.93 |
| WV-4158 | 0.3 | 0.44 | | 0.91 | 0.54 | 0.93 | 0.82 | 1.22 |
| | 0.17 | 0.42 | 0.72 | 0.98 | | 1.07 | 1.11 | 1.01 |
| WV-4169 | 0.46 | 0.86 | 0.92 | 0.69 | 0.98 | 0.91 | 1.11 | 1.17 |
| | 0.34 | 0.63 | 0.96 | 1.12 | 0.78 | 1.09 | | 0.95 |
| WV-4146 | 0.27 | 0.56 | 0.72 | 0.83 | 0.84 | 0.86 | | 1.01 |
| | 0.16 | 0.45 | | 1.15 | 0.76 | 0.98 | 0.87 | 0.98 |
| WV-4179 | 0.56 | 0.75 | 1.12 | 1.15 | | | 0.75 | 1.04 |
| | 0.19 | 0.64 | 0.8 | 0.84 | 0.87 | 1.12 | 1.01 | 0.8 |
| WV-4157 | 0.2 | 0.33 | 0.72 | 0.85 | 1.25 | 0.87 | 1.03 | 0.99 |
| | 0.1 | 0.31 | 0.55 | 1.04 | 0.74 | 1.48 | 0.77 | 1.15 |

| Log Conc. (nM) | 25 | 6.25 | 1.5625 | 0.390625 | 0.097656 | 0.024414 | 0.006104 | 0.001526 |
|---|---|---|---|---|---|---|---|---|
| H2O | 0.78 | 0.75 | 0.78 | 0.95 | 0.7 | 0.84 | 1.01 | 1.06 |
| | 0.68 | 0.79 | 0.84 | 0.98 | 0.56 | | 0.57 | 0.68 |
| WV-4186 | 0.06 | 0.09 | 0.12 | 0.15 | 0.61 | 0.77 | 0.82 | 0.98 |
| | 0.05 | 0.12 | 0.14 | 0.23 | 0.38 | 0.69 | 1.17 | 0.83 |
| WV-4258 | 0.29 | 0.56 | 0.71 | 0.75 | 0.81 | 0.98 | 1.01 | 1.25 |
| | 0.34 | 0.45 | 0.87 | | 0.88 | 0.82 | | 0.92 |
| WV-4248 | 0.79 | 0.69 | 0.82 | 0.77 | 0.83 | 0.86 | 0.87 | 1.01 |
| | 0.93 | 0.87 | 0.77 | | 0.8 | 1.07 | 0.97 | 1.02 |
| WV-4263 | 0.72 | 0.64 | 0.85 | 0.93 | 0.95 | 1.01 | 1.03 | 0.91 |
| | 0.81 | 0.73 | | 0.9 | | 0.86 | 0.27 | 1 |
| WV-4253 | 0.75 | 0.84 | 0.88 | 0.85 | 0.83 | 0.71 | 0.65 | 0.98 |
| | 0.92 | 0.82 | 0.73 | 0.85 | 1.02 | 0.98 | 1.03 | 1.04 |
| WV-4161 | 0.22 | 0.22 | 0.39 | 0.65 | 1.03 | 0.99 | 0.71 | 1.08 |
| | 0.17 | 0.22 | 0.35 | 0.56 | 0.93 | 0.96 | 0.94 | 1 |
| WV-2817 | 0.2 | 0.23 | 0.34 | 1.26 | 0.79 | 0.82 | 1.05 | 1.12 |
| | 0.19 | 0.24 | 0.33 | 0.64 | 0.68 | | 0.94 | 1 |
| WV-3122 | 0.19 | 0.16 | 0.31 | | 0.79 | 0.78 | 1.08 | 0.96 |
| | 0.11 | 0.18 | 0.36 | 0.44 | 0.54 | 0.81 | 0.82 | 0.88 |
| WV-3021 | 0.28 | | 0.28 | 0.45 | 0.61 | 0.94 | 1.07 | 1.06 |
| | | 0.17 | 0.31 | 0.47 | 0.64 | 0.86 | 0.9 | 1.08 |
| WV-4171 | 0.32 | 0.62 | 0.86 | | 1 | 0.61 | 1.01 | 1.08 |
| | 0.23 | 0.69 | 1.08 | | 1.12 | 1.05 | 0.91 | 0.93 |
| WV-4148 | 0.32 | 0.52 | 0.92 | 0.83 | 1.26 | 0.73 | | 1.01 |
| | 0.22 | 0.59 | 1.16 | 1.1 | 0.86 | 1.12 | | 1.13 |

As shown in Tables 48 to 60, various oligonucleotides were constructed and tested for their ability to mediate knockdown of APOC3, including in vitro. Without wishing to be bound by any theory, the present disclosure suggests that at least some of the oligonucleotides in Tables 48 to 60 may be capable of mediating knockdown via a RNaseH-mediated mechanism. FIG. 2 shows example formats of oligonucleotides. In some embodiments, oligonucleotides of these example formats can be RNase-H dependent antisense oligonucleotides (ASOs). In addition, some of the oligonucleotides in these tables have a hybrid format.

TABLE 48
In vitro efficacy of different ASOs which target APOC3.

| | 3 nM | 30 nM | Oligonucleotide | 3 nM | 30 nM |
|---|---|---|---|---|---|
| WV-753 332 | 0.690 | 0.075 | WV-747 151 | 0.773 | 0.464 |
| WV-744 499 | 0.634 | 0.111 | WV-705 210 | 0.803 | 0.467 |
| WV-754 333 | 0.581 | 0.112 | WV-759 142 | 0.775 | 0.469 |
| WV-742 225 | 0.576 | 0.125 | WV-772 149 | 0.905 | 0.473 |
| WV-755 334 | 0.568 | 0.150 | WV-703 159 | 0.823 | 0.480 |
| WV-743 226 | 0.607 | 0.161 | WV-706 81 | 0.831 | 0.480 |
| WV-737 193 | 0.744 | 0.161 | WV-704 160 | 0.774 | 0.483 |
| WV-769 168 | 0.695 | 0.176 | WV-709 109 | 0.769 | 0.488 |
| WV-722 267 | 0.842 | 0.179 | WV-774 115 | 0.734 | 0.490 |
| WV-738 221 | 0.634 | 0.182 | WV-702 158 | 0.771 | 0.491 |
| WV-715 330 | 0.601 | 0.182 | WV-765 319 | 0.889 | 0.493 |
| WV-721 230 | 0.766 | 0.190 | WV-713 153 | 0.825 | 0.512 |
| WV-741 224 | 0.685 | 0.200 | WV-762 310 | 0.859 | 0.528 |
| WV-723 501 | 0.542 | 0.204 | WV-711 111 | 0.897 | 0.533 |
| WV-750 186 | 0.758 | 0.210 | WV-710 110 | 0.801 | 0.536 |
| WV-749 183 | 0.691 | 0.217 | WV-776 2126 Intron | 0.834 | 0.539 |
| WV-720 229 | 0.687 | 0.232 | WV-764 318 | 0.899 | 0.542 |
| WV-752 188 | 0.759 | 0.233 | WV-763 312 | 0.905 | 0.558 |
| WV-739 222 | 0.725 | 0.236 | WV-708 108 | 0.889 | 0.559 |
| WV-714 155 | 0.789 | 0.240 | WV-696 162 | 1.043 | 0.561 |
| WV-767 321 | 0.767 | 0.246 | WV-712 112 | 0.736 | 0.570 |
| WV-717 165 | 0.716 | 0.258 | WV-693 13 | 0.748 | 0.588 |
| WV-751 187 | 0.782 | 0.260 | WV-775 314 | 0.856 | 0.600 |
| WV-736 190 | 0.743 | 0.261 | WV-778 557 Intron | 0.757 | 0.603 |
| WV-698 164 | 0.526 | 0.264 | WV-716 484 | 0.874 | 0.626 |
| WV-740 223 | 0.697 | 0.292 | WV-733 100 | 0.821 | 0.634 |
| WV-718 166 | 0.705 | 0.302 | WV-692 12 | 0.946 | 0.634 |
| WV-735 172 | 0.731 | 0.308 | WV-746 106 | 0.841 | 0.657 |
| WV-757 396 | 0.698 | 0.341 | WV-773 143 | 0.862 | 0.676 |
| WV-701 157 | 0.734 | 0.346 | WV-770 5 | 0.857 | 0.715 |
| WV-766 320 | 0.832 | 0.348 | WV-724 4 | 0.943 | 0.729 |
| WV-697 163 | 0.780 | 0.353 | WV-777 2593 Intron | 0.801 | 0.762 |
| WV-760 306 | 0.773 | 0.367 | WV-730 97 | 0.967 | 0.764 |
| WV-699 206 | 0.814 | 0.369 | WV-771 105 | 0.922 | 0.784 |
| WV-768 152 | 0.857 | 0.369 | WV-745 103 | 1.064 | 0.789 |
| WV-758 73 | 0.867 | 0.384 | WV-694 19 | 0.782 | 0.792 |
| WV-695 161 | 0.720 | 0.400 | WV-707 107 | 0.951 | 0.805 |
| WV-756 394 | 0.780 | 0.406 | WV-734 101 | 0.898 | 0.818 |
| WV-748 177 | 0.734 | 0.411 | WV-732 99 | 0.819 | 0.871 |
| WV-700 156 | 0.884 | 0.423 | WV-731 98 | 0.856 | 0.952 |
| WV-726 36 | 0.728 | 0.425 | WV-729 95 | 1.045 | 1.210 |
| WV-725 33 | 0.736 | 0.436 | WV-728 94 | 0.898 | 1.281 |
| WV-761 307 | 0.962 | 0.458 | WV-727 93 | 0.842 | 1.541 |
| WV-719 201 | 0.673 | 0.460 | | | |

The oligonucleotide ID (identity) and position APOC3 is given. For example, WV-753 332 indicates oligonucleotide WV-753 at position 332 in the APOC3 gene. Oligonucleotides tested are: WV-692 to WV-777. In this table and various other tables, numbers indicate level of mRNA of APOC3/GAPDH, relative to untreated control, wherein 1 would represent no knockdown of APOC3 mRNA and 0 would represent 100% knockdown. Some oligonucleotides are not to target an intron of APOC3.

TABLE 49
In vitro efficacy of different ASOs, which target APOC3.

| Oligonucleotide | 3 nM | 30 nM |
|---|---|---|
| WV-840 499 | 0.387 | 0.099 |
| WV-849 332 | 0.448 | 0.109 |
| WV-853 396 | 0.668 | 0.123 |
| WV-838 225 | 0.356 | 0.146 |
| WV-852 394 | 0.650 | 0.166 |
| WV-839 226 | 0.455 | 0.167 |
| WV-837 224 | 0.466 | 0.172 |
| WV-833 193 | 0.681 | 0.174 |
| WV-831 172 | 0.570 | 0.181 |
| WV-819 501 | 0.319 | 0.189 |
| WV-847 187 | 0.553 | 0.204 |
| WV-848 188 | 0.620 | 0.210 |
| WV-845 183 | 0.586 | 0.225 |
| WV-863 321 | 0.632 | 0.227 |
| WV-791 161 | 0.843 | 0.229 |
| WV-811 330 | 0.576 | 0.244 |
| WV-836 223 | 0.379 | 0.245 |
| WV-801 210 | 0.826 | 0.246 |
| WV-832 190 | 0.695 | 0.250 |
| WV-818 267 | 0.730 | 0.251 |
| WV-813 165 | 0.707 | 0.251 |
| WV-834 211 | 0.525 | 0.256 |
| WV-816 229 | 0.549 | 0.256 |
| WV-850 333 | 0.508 | 0.257 |
| WV-864 152 | 0.714 | 0.265 |
| WV-817 230 | 0.613 | 0.267 |
| WV-856 306 | 0.566 | 0.279 |
| WV-862 320 | 0.647 | 0.280 |
| WV-861 319 | 0.751 | 0.288 |
| WV-854 73 | 0.605 | 0.289 |
| WV-844 177 | 0.777 | 0.299 |
| WV-865 168 | 0.552 | 0.304 |
| WV-799 159 | 0.802 | 0.307 |
| WV-857 307 | 0.681 | 0.315 |
| WV-792 162 | 0.917 | 0.320 |
| WV-815 201 | 0.679 | 0.330 |
| WV-846 186 | 0.576 | 0.330 |
| WV-800 160 | 0.940 | 0.331 |
| WV-812 484 | 0.684 | 0.335 |
| WV-835 222 | 0.455 | 0.336 |
| WV-793 163 | 0.906 | 0.349 |
| WV-814 166 | 0.714 | 0.350 |
| WV-860 318 | 0.690 | 0.370 |
| WV-797 157 | 0.841 | 0.372 |
| WV-794 164 | 0.613 | 0.379 |
| WV-851 334 | 0.477 | 0.387 |
| WV-871 314 | 0.628 | 0.394 |
| WV-807 111 | 0.795 | 0.400 |
| WV-809 153 | 0.768 | 0.405 |
| WV-822 36 | 0.593 | 0.418 |
| WV-806 110 | 0.741 | 0.428 |
| WV-868 149 | 0.871 | 0.442 |
| WV-805 109 | 0.761 | 0.442 |
| WV-870 115 | 0.741 | 0.442 |
| WV-855 142 | 0.687 | 0.453 |
| WV-810 155 | 0.729 | 0.454 |
| WV-796 156 | 0.677 | 0.454 |
| WV-802 81 | 0.901 | 0.469 |
| WV-798 158 | 0.799 | 0.475 |
| WV-808 112 | 0.754 | 0.475 |
| WV-858 310 | 0.712 | 0.480 |
| WV-789 13 | 0.826 | 0.494 |
| WV-821 33 | 0.652 | 0.501 |
| WV-788 12 | 0.763 | 0.520 |
| WV-804 108 | 0.692 | 0.534 |
| WV-820 4 | 0.774 | 0.553 |
| WV-872 2126 Intron | 0.674 | 0.558 |
| WV-874 557 Intron | 0.634 | 0.560 |
| WV-829 100 | 0.909 | 0.586 |
| WV-795 206 | 0.876 | 0.600 |
| WV-842 1076 | 0.752 | 0.617 |
| WV-866 5 | 0.767 | 0.622 |
| WV-830 101 | 0.809 | 0.636 |
| WV-843 151 | 0.705 | 0.657 |
| WV-873 2593 Intron | 0.883 | 0.672 |
| WV-869 143 | 0.647 | 0.680 |
| WV-841 103 | 0.974 | 0.708 |
| WV-803 107 | 0.815 | 0.716 |
| WV-790 19 | 0.942 | 0.784 |
| WV-859 312 | 0.729 | 0.785 |
| WV-867 105 | 0.756 | 0.838 |
| WV-826 97 | 0.757 | 0.876 |
| WV-828 99 | 0.630 | 0.885 |
| WV-827 98 | 0.719 | 1.015 |
| WV-825 95 | 1.070 | 1.121 |
| WV-823 93 | 1.013 | 1.289 |
| WV-824 94 | 0.846 | 1.331 |

The oligonucleotide ID (identity) and position APOC3 is given. For example, WV-840 499 indicates oligonucleotide WV-840 at position 499 in the APOC3 gene. Oligonucleotides tested are: WV-788 to WV-873.

TABLE 50
In vitro efficacy of different ASOs which target APOC3.

| | 30 nM | 3 nM |
|---|---|---|
| WV-1434″172 | 0.125 | 0.683 |
| WV-1443″499 | 0.169 | 0.419 |
| WV-1437″221 | 0.171 | 0.620 |
| WV-1453″333 | 0.174 | 0.539 |
| WV-1414″330 | 0.178 | 0.515 |
| WV-1456″396 | 0.178 | 0.655 |
| WV-1435″190 | 0.182 | 0.646 |
| WV-1419″229 | 0.195 | 0.664 |
| WV-1448″183 | 0.195 | 0.619 |
| WV-840 5-15 499 | 0.198 | 0.482 |
| WV-1442″226 | 0.208 | 0.615 |
| WV-1455″394 | 0.213 | 0.671 |
| WV-1440″224 | 0.219 | 0.523 |
| WV-1451″188 | 0.224 | 0.626 |
| WV-1439″223 | 0.228 | 0.539 |
| WV-1459″306 | 0.229 | 0.697 |
| WV-1454″334 | 0.231 | 0.641 |
| WV-1421″267 | 0.233 | 0.627 |
| WV-1395″162 | 0.237 | 0.797 |
| WV-1466″321 | 0.238 | 0.598 |
| WV-1450″187 | 0.241 | 0.769 |
| WV-1422″501 | 0.243 | 0.525 |
| WV-1402″159 | 0.258 | 0.658 |
| WV-1405″81 | 0.261 | 0.714 |
| WV-1416″165 | 0.268 | 0.549 |
| WV-1418″201 | 0.275 | 0.658 |
| WV-1438″222 | 0.277 | 0.501 |
| WV-744 AIIDNA 499 | 0.288 | 0.896 |
| WV-1458″142 | 0.290 | 0.588 |
| WV-1394″161 | 0.292 | 0.581 |
| WV-1424″33 | 0.296 | 0.519 |
| WV-1474″314 | 0.300 | 0.584 |
| WV-1461″310 | 0.310 | 0.718 |
| WV-1397″164 | 0.313 | 0.767 |
| WV-1411″112 | 0.314 | 0.584 |
| WV-1403″160 | 0.321 | 0.591 |
| WV-1467″152 | 0.325 | 0.926 |
| WV-1464″319 | 0.326 | 0.524 |
| WV-1410″111 | 0.329 | 0.426 |
| WV-1413″155 | 0.337 | 0.714 |
| WV-1408″109 | 0.364 | 0.753 |
| WV-1447″177 | 0.365 | 0.759 |
| WV-1400″157 | 0.371 | 0.604 |
| WV-1477″557 Int | 0.386 | 0.790 |
| WV-1398″206 | 0.397 | 0.619 |
| WV-1399″156 | 0.410 | 0.753 |
| WV-1472″143 | 0.418 | 0.776 |
| WV-1475″2126 Int | 0.419 | 0.881 |
| WV-1407″108 | 0.441 | 0.669 |
| WV-1415″484 | 0.442 | 0.703 |
| WV-1462″312 | 0.446 | 0.833 |
| WV-1463″318 | 0.458 | 1.022 |
| WV-1471″149 | 0.468 | 0.963 |
| WV-1445″106 | 0.473 | 0.884 |
| WV-1469″5 | 0.490 | 0.817 |
| WV-1479″792 Int | 0.514 | 0.981 |
| WV-1391 ″12 | 0.530 | 0.833 |
| WV-1392 ″13 | 0.534 | 0.758 |
| WV-1480″793 Int | 0.546 | 0.982 |
| WV-1406″107 | 0.559 | 0.966 |
| WV-1432″100 | 0.560 | 0.745 |
| WV-1423″4 | 0.571 | 0.805 |
| WV-1449″186 | 0.578 | 0.717 |
| WV-1427″94 | 0.619 | 0.872 |
| WV-1452″332 | 0.628 | 0.876 |
| WV-1425″36 | 0.629 | 0.865 |
| WV-1465″320 | 0.630 | 0.762 |
| WV-1404″210 | 0.643 | 0.770 |
| WV-1393″19 | 0.645 | 0.879 |
| WV-1441″225 | 0.648 | 0.720 |
| WV-1401″158 | 0.652 | 0.770 |
| WV-1431″99 | 0.662 | 0.796 |
| WV-1417″166 | 0.664 | 0.720 |
| WV-1409″110 | 0.668 | 0.838 |
| WV-1446″151 | 0.669 | 0.928 |
| WV-1444″103 | 0.669 | 0.823 |
| WV-1429″97 | 0.675 | 0.894 |
| WV-1428″95 | 0.690 | 0.874 |
| WV-1412″153 | 0.693 | 0.803 |
| WV-1473″115 | 0.696 | 0.668 |
| WV-1476″2593 Int | 0.697 | 1.073 |
| WV-1420″230 | 0.711 | 0.525 |
| WV-1396″163 | 0.718 | 0.880 |
| WV-1436″193 | 0.719 | 0.970 |
| WV-1433″101 | 0.724 | 0.395 |
| WV-1457″73 | 0.729 | 1.001 |
| WV-1426″93 | 0.760 | 0.832 |
| WV-1468″168 | 0.769 | 0.971 |
| WV-1430″98 | 0.782 | 0.829 |
| WV-1470″105 | 0.800 | 0.618 |
| WV-1460″307 | 0.804 | 0.880 |
| WV-1481″796 Int | 0.810 | 1.319 |
| TR Only″ | 0.974 | 0.961 |
| WV-1478″780 Int | 0.992 | 1.403 |
| TR Only″ | 1.026 | 1.039 |

The oligonucleotide ID (identity) and position in APOC3 are provided. For example, WV-1434 172 indicates oligonucleotide WV-1434 at position 172 in the APOC3 gene (wherein the oligonucleotide designation and position are separated by ″).

TABLE 51
In vitro efficacy of different ASOs, which target APOC3.

| | 3 nM | 30 nM | 3 nM | 30 nM |
|---|---|---|---|---|
| WV-779 780 | 0.981 | 0.695 | 0.113 | 0.013 |
| WV-780 792 | 0.921 | 0.623 | 0.096 | 0.003 |
| WV-781 793 | 0.877 | 0.568 | 0.061 | 0.049 |
| WV-782 796 | 0.818 | 0.546 | 0.071 | 0.060 |
| WV-783 1130 | 0.886 | 0.709 | 0.086 | 0.057 |
| WV-784 2649 | 0.872 | 0.419 | 0.139 | 0.022 |
| WV-785 2734 | 0.876 | 0.756 | 0.115 | 0.056 |
| WV-786 2771 | 0.871 | 0.770 | 0.017 | 0.017 |
| WV-787 2803 | 0.896 | 0.681 | 0.003 | 0.042 |
| WV-875 | 0.922 | 0.559 | 0.139 | 0.014 |
| WV-876 | 0.782 | 0.493 | 0.052 | 0.007 |
| WV-877 | 0.865 | 0.510 | 0.014 | 0.024 |
| WV-878 | 0.717 | 0.462 | 0.232 | 0.025 |
| WV-879 | 0.887 | 0.795 | 0.027 | 0.020 |
| WV-880 | 0.719 | 0.361 | 0.152 | 0.020 |
| WV-881 | 0.888 | 0.790 | 0.053 | 0.084 |
| WV-882 | 0.909 | 0.665 | 0.197 | 0.060 |
| WV-883 | 0.900 | 0.630 | 0.100 | 0.079 |

The oligonucleotide ID (identity) and position in APOC3 are provided. FOXO1 ASOs were also tested (data not shown).

TABLE 52
In vitro efficacy of different ASOs, which target APOC3.

| | | | |
|---|---|---|---|
| Conc (exp | | | |
| 10) (nM) | WV-744 499 | WV-753 332 | WV-742 225 |

| | | | | | | |
|---|---|---|---|---|---|---|
| 1.778 | 0.11 | −0.01 | −0.07 | −0.06 | −0.08 | −0.01 |
| 1.301 | 0.18 | 0.17 | 0.07 | 0.02 | 0.06 | 0.01 |
| 0.824 | 0.47 | 0.32 | 0.32 | 0.27 | 0.24 | 0.15 |
| 0.347 | 0.76 | 0.59 | 0.64 | 0.59 | 0.66 | 0.57 |
| −0.130 | 1.01 | 0.90 | 0.88 | 0.73 | 0.91 | 0.65 |
| −0.607 | 1.26 | 0.99 | 0.95 | 0.81 | 0.97 | 0.80 |
| −1.085 | 1.02 | 1.02 | 1.03 | 0.85 | 0.90 | 0.94 |
| −1.562 | | | | | | |

| | | | |
|---|---|---|---|
| Conc (exp | | | |
| 10) (nM) | WV-737 193 | WV-840 499 | WV-849 332 |

| | | | | | | |
|---|---|---|---|---|---|---|
| 1.778 | −0.01 | −0.06 | −0.04 | −0.03 | −0.05 | −0.08 |
| 1.301 | 0.09 | 0.09 | 0.01 | 0.01 | 0.05 | 0.05 |
| 0.824 | 0.57 | 0.38 | 0.18 | 0.11 | 0.16 | 0.16 |
| 0.347 | 0.82 | 0.76 | 0.37 | 0.32 | 0.44 | 0.41 |
| −0.130 | 0.91 | 0.85 | 0.65 | 0.57 | 0.73 | 0.68 |
| −0.607 | 1.07 | 1.06 | 1.04 | 0.76 | 1.09 | 0.89 |
| −1.085 | 1.02 | 1.07 | 0.92 | 0.97 | 1.00 | 0.83 |
| −1.562 | | | | | | |

| | | | |
|---|---|---|---|
| Conc (exp | | | |
| 10) (nM) | WV-838 225 | WV-833 193 | WV-1443 499 |

| | | | | | | |
|---|---|---|---|---|---|---|
| 1.778 | −0.07 | −0.07 | 0.01 | 0.00 | 0.10 | 0.19 |
| 1.301 | 0.04 | −0.01 | 0.09 | 0.06 | 0.32 | 0.21 |
| 0.824 | 0.13 | 0.09 | 0.34 | 0.27 | 0.30 | 0.26 |
| 0.347 | 0.40 | 0.30 | 0.68 | 0.61 | 0.43 | 0.45 |
| −0.130 | 0.71 | 0.74 | 0.94 | 0.98 | 0.81 | 0.64 |
| −0.607 | 1.00 | 0.95 | 1.16 | 0.95 | 0.95 | 0.71 |
| −1.085 | 1.08 | 1.06 | 1.09 | 0.99 | 0.88 | 0.92 |
| −1.562 | | | | | 1.04 | 1.19 |

| | | | |
|---|---|---|---|
| Conc (exp | | | |
| 10) (nM) | WV-1452 332 | WV-1441 225 | WV-1436 193 |

| | | | | | | |
|---|---|---|---|---|---|---|
| 1.778 | 0.02 | 0.01 | 0.13 | 0.05 | 0.07 | 0.09 |
| 1.301 | 0.10 | 0.10 | 0.18 | 0.15 | 0.28 | 0.26 |
| 0.824 | 0.27 | 0.34 | 0.37 | 0.31 | 0.57 | 0.68 |
| 0.347 | 0.63 | 0.44 | 0.73 | 0.55 | 0.78 | 0.82 |
| −0.130 | 0.86 | 0.80 | 0.89 | 0.82 | 0.84 | 0.97 |
| −0.607 | 1.11 | 0.88 | 1.07 | 1.00 | 1.12 | 0.95 |
| −1.085 | 0.92 | 0.93 | 0.94 | 0.95 | 0.72 | 1.05 |
| −1.562 | 1.43 | 0.89 | 1.27 | 0.96 | 0.83 | 1.03 |

| | |
|---|---|
| Conc (exp | |
| 10) (nM) | WV-437 |

| | | |
|---|---|---|
| 1.778 | 1.37 | 1.43 |
| 1.301 | 1.21 | 1.18 |
| 0.824 | 0.99 | 1.03 |
| 0.347 | 1.25 | 1.25 |
| −0.130 | 1.14 | 1.06 |
| −0.607 | 1.49 | 1.20 |
| −1.085 | 1.46 | 1.44 |
| −1.562 | | |

| | WV-744 499 | WV-753 332 | WV-742 225 | WV-737 193 | WV-840 499 | WV-849 332 |
|---|---|---|---|---|---|---|
| 1.778 | 0.08 | 0.01 | 0.05 | 0.04 | 0.00 | 0.02 |
| 1.301 | 0.01 | 0.04 | 0.03 | 0.00 | 0.01 | 0.00 |
| 0.824 | 0.11 | 0.03 | 0.06 | 0.14 | 0.05 | 0.00 |
| 0.347 | 0.12 | 0.03 | 0.06 | 0.04 | 0.03 | 0.02 |
| −0.130 | 0.08 | 0.11 | 0.19 | 0.04 | 0.06 | 0.04 |
| −0.607 | 0.19 | 0.09 | 0.12 | 0.01 | 0.20 | 0.14 |
| −1.085 | 0.00 | 0.13 | 0.03 | 0.04 | 0.04 | 0.12 |

| | WV-838 225 | WV-833 193 | WV-1443 499 | WV-1452 332 | WV-1441 225 | WV-1436 193 | WV-437 |
|---|---|---|---|---|---|---|---|
| 1.778 | 0.00 | 0.01 | 0.07 | 0.01 | 0.05 | 0.02 | 0.04 |
| 1.301 | 0.04 | 0.02 | 0.08 | 0.01 | 0.02 | 0.02 | 0.02 |
| 0.824 | 0.03 | 0.05 | 0.03 | 0.05 | 0.04 | 0.08 | 0.02 |
| 0.347 | 0.07 | 0.05 | 0.01 | 0.13 | 0.13 | 0.03 | 0.00 |
| −0.130 | 0.02 | 0.03 | 0.12 | 0.05 | 0.05 | 0.10 | 0.05 |
| −0.607 | 0.04 | 0.15 | 0.17 | 0.16 | 0.05 | 0.12 | 0.20 |
| −1.085 | 0.02 | 0.07 | 0.03 | 0.01 | 0.01 | 0.24 | 0.01 |
| −1.562 | | | 0.10 | 0.39 | 0.22 | 0.14 | |

The oligonucleotide ID (identity) and position in APOC3 are provided.

TABLE 53
In vitro efficacy screening of different ASOs which target APOC3.

| | 30 nM | 10 nM | 3 nM | | | | |
|---|---|---|---|---|---|---|---|
| WV-1863 330 | 0.068 | 0.128 | 0.303 | WV-1874 223 | 0.190 | 0.215 | 0.362 |
| WV-1878 499 | 0.072 | 0.122 | 0.204 | WV-1875 224 | 0.197 | 0.257 | 0.326 |
| WV-1864 165 | 0.076 | 0.219 | 0.566 | WV-1852 164 | 0.202 | 0.272 | 0.659 |
| WV-1886 394 | 0.079 | 0.184 | 0.360 | WV-1856 159 | 0.209 | 0.489 | 0.732 |
| WV-1870 172 | 0.083 | 0.120 | 0.236 | WV-1851 162 | 0.233 | 0.537 | 0.846 |
| WV-1887 395 | 0.086 | 0.287 | 0.587 | WV-1858 81 | 0.247 | 0.296 | 0.667 |
| WV-1868 501 | 0.094 | 0.091 | 0.141 | WV-1888 142 | 0.248 | 0.522 | 0.716 |
| WV-1883 332 | 0.104 | 0.182 | 0.271 | WV-1865 201 | 0.248 | 0.342 | 0.724 |
| WV-1885 334 | 0.109 | 0.255 | 0.325 | WV-1890 310 | 0.252 | 0.645 | 0.626 |
| WV-1884 333 | 0.118 | 0.223 | 0.352 | WV-1869 33 | 0.258 | 0.380 | 0.578 |
| WV-1876 225 | 0.126 | 0.257 | 0.343 | WV-1850 161 | 0.258 | 0.444 | 0.781 |
| WV-1880 183 | 0.135 | 0.354 | 0.541 | WV-1893 152 | 0.267 | 0.695 | 0.630 |
| WV-1867 267 | 0.135 | 0.315 | 0.464 | WV-1891 319 | 0.284 | 0.553 | 0.632 |
| WV-1871 190 | 0.138 | 0.164 | 0.410 | WV-1857 160 | 0.285 | 0.525 | 1.059 |
| WV-1873 222 | 0.147 | 0.251 | 0.383 | WV-1862 155 | 0.294 | 0.635 | 0.645 |
| WV-1872 221 | 0.149 | 0.237 | 0.367 | WV-1855 157 | 0.308 | 0.611 | 0.743 |
| WV-1889 306 | 0.160 | 0.284 | 0.550 | WV-1853 206 | 0.328 | 0.405 | 0.875 |
| WV-1879 177 | 0.168 | 0.370 | 0.596 | WV-1860 111 | 0.337 | 0.727 | 0.652 |
| WV-1892 321 | 0.169 | 0.485 | 0.618 | WV-1854 156 | 0.337 | 0.628 | 0.685 |
| WV-1881 187 | 0.174 | 0.218 | 0.390 | WV-1861 112 | 0.373 | 0.568 | 0.603 |
| WV-1882 188 | 0.182 | 0.350 | 0.586 | WV-1859 109 | 0.456 | 0.627 | 0.644 |
| WV-1865 229 | 0.186 | 0.261 | 0.415 | Control | 1.013 | 1.191 | 0.806 |
| | | | | WV-744 | 0.070 | 0.207 | 0.381 |
| | | | | WV-840 | 0.051 | 0.115 | 0.169 |
| | | | | WV-1443 | 0.205 | 0.246 | 0.194 |

The oligonucleotide ID (identity) and position in APOC3 are provided.

Table 54 shows in vitro efficacy of different ASOs, which target APOC3. Oligonucleotides tested are: WV-1868, WV-1878, WV-1887, WV-1886, WV-1885, WV-1884, WV-1883, WV-1863, WV-1876, WV-1871, WV-1870 and WV-1864.

TABLE 54
Activity of oligonucleotides.

| Conc (exp | | | | | | | | |
|---|---|---|---|---|---|---|---|---|
| 10) (nM) | WV-1868 | | WV-1878 | | WV-1887 | | WV-1886 | |
| 1.477 | 0.114 | 0.072 | 0.138 | 0.100 | 0.098 | 0.123 | 0.182 | 0.123 |
| 1.000 | 0.150 | 0.102 | 0.119 | 0.142 | 0.321 | 0.405 | 0.175 | 0.357 |
| 0.523 | 0.208 | 0.222 | 0.191 | 0.264 | 0.556 | 1.242 | 0.448 | 0.860 |
| 0.046 | 0.307 | 0.372 | 0.521 | 0.539 | 0.735 | 0.986 | 0.785 | 1.236 |
| −0.431 | 0.386 | 0.441 | 0.536 | 0.784 | 0.770 | 1.003 | 0.714 | 0.734 |
| −0.908 | 0.578 | 0.817 | 0.747 | 0.793 | 0.808 | 1.253 | 0.996 | 0.898 |
| −1.386 | 0.715 | 0.808 | 0.742 | 1.476 | 0.888 | 0.851 | 0.869 | 1.028 |
| Conc (exp | | | | | | | | |
| 10) (nM) | WV-1885 | | WV-1884 | | WV-1883 | | WV-1863 | |
| 1.477 | 0.156 | 0.148 | 0.200 | 0.150 | 0.138 | 0.085 | 0.097 | 0.074 |
| 1.000 | 0.254 | 0.358 | 0.294 | 0.293 | 0.261 | 0.198 | 0.180 | 0.207 |
| 0.523 | 0.353 | 0.553 | 0.391 | 0.554 | 0.413 | 0.399 | 0.277 | 0.430 |
| 0.046 | 0.590 | 0.776 | 0.548 | 0.748 | 0.531 | 0.611 | 0.553 | 0.731 |
| −0.431 | 0.703 | 0.939 | 0.582 | 0.795 | 0.794 | 0.635 | 0.575 | 0.731 |
| −0.908 | 0.797 | 0.846 | 0.769 | 0.702 | 0.910 | 0.694 | 0.846 | 0.921 |
| −1.386 | 0.747 | 1.063 | 0.787 | 0.828 | 0.831 | 0.797 | 0.725 | 1.137 |
| Conc (exp | | | | | | | | |
| 10) (nM) | WV-1876 | | WV-1871 | | WV-1870 | | WV-1864 | |
| 1.477 | 0.168 | 0.115 | 0.103 | 0.109 | 0.131 | 0.112 | 0.104 | 0.121 |
| 1.000 | 0.275 | 0.375 | 0.217 | 0.277 | 0.132 | 0.223 | 0.267 | 0.379 |
| 0.523 | 0.304 | 0.564 | 0.315 | 0.844 | 0.333 | 0.536 | 0.671 | 0.669 |
| 0.046 | 0.461 | 0.883 | 0.590 | 1.093 | 0.630 | 0.924 | 0.819 | 0.817 |
| −0.431 | 0.613 | 0.763 | 0.662 | 0.825 | 0.710 | 0.916 | 0.916 | 1.008 |
| −0.908 | 0.551 | 0.783 | 0.844 | 0.914 | 0.866 | 0.964 | 1.005 | 0.758 |
| −1.386 | 0.903 | 1.188 | 0.816 | 1.542 | 0.759 | 0.924 | 0.906 | 0.946 |

Table 55. Table 55 shows the IC50 of different ASOs, which target APOC3. Oligonucleotides tested are: WV-723, WV-819, WV-1422, and WV-1868.

TABLE 55
Activity of oligonucleotides.

| Oligonucleotide | IC50 (nM) |
|---|---|
| WV-723 | 1.5 |
| WV-819 | 0.5 |
| WV-1422 | 0.9 |
| WV-1868 | 1.4 |

Table 56. Table 56 shows in vitro efficacy of different ASOs, which target APOC3. Oligonucleotides tested are: WV-2115 to WV-2124, WV-2126, and WV-1422. The oligonucleotides have different but overlapping base sequences.

TABLE 56
Activity of oligonucleotides.

| | | | |
|---|---|---|---|
| Conc (exp | | | |
| 10) (nM) | WV-2115 | WV-2116 | WV-2117 |

| | | | | | | |
|---|---|---|---|---|---|---|
| 1.477 | 0.169 | 0.160 | 0.165 | 0.191 | 0.150 | 0.271 |
| 1.000 | 0.164 | 0.320 | 0.219 | 0.194 | 0.404 | 0.264 |
| 0.523 | 0.298 | 0.477 | 0.258 | 0.419 | 0.879 | 0.549 |
| 0.046 | 0.495 | 0.562 | 0.615 | 0.662 | 1.078 | 0.724 |
| −0.431 | 0.589 | 0.699 | 0.394 | 0.706 | 1.176 | 0.685 |
| −0.908 | 0.732 | 0.784 | 0.708 | 0.793 | 0.929 | 0.886 |
| −1.386 | 0.972 | 0.832 | 0.958 | 0.812 | 1.292 | 0.871 |
| −1.863 | 0.887 | 0.924 | 1.051 | 1.019 | 1.014 | 1.126 |

| | | | |
|---|---|---|---|
| Conc (exp | | | |
| 10) (nM) | WV-2118 | WV-2119 | WV-2120 |

| | | | | | | |
|---|---|---|---|---|---|---|
| 1.477 | 0.350 | 0.242 | 0.285 | 0.254 | 0.442 | 0.323 |
| 1.000 | 0.521 | 0.310 | 0.358 | 0.297 | 0.651 | 0.246 |
| 0.523 | 0.936 | 0.534 | 0.877 | 0.811 | 0.548 | 0.477 |
| 0.046 | 1.808 | 0.629 | 1.252 | 1.005 | 1.096 | 0.706 |
| −0.431 | 1.296 | 0.777 | 1.204 | 0.900 | 1.033 | 0.722 |
| −0.908 | 0.869 | 1.080 | 0.737 | 0.761 | 0.951 | 0.735 |
| −1.386 | 0.865 | 0.830 | 0.910 | 1.009 | 0.946 | 0.809 |
| −1.863 | 0.968 | 1.029 | 1.318 | 1.071 | 1.094 | 0.864 |

| | | | |
|---|---|---|---|
| Conc (exp | | | |
| 10) (nM) | WV-2121 | WV-2122 | WV-2123 |

| | | | | | | |
|---|---|---|---|---|---|---|
| 1.477 | 0.528 | 0.355 | 0.316 | 0.267 | 0.257 | 0.260 |
| 1.000 | 0.339 | 0.228 | 0.415 | 0.212 | 0.377 | 0.298 |
| 0.523 | 0.622 | 0.349 | 0.553 | 0.439 | 0.310 | 0.347 |
| 0.046 | 0.808 | 0.687 | 1.331 | 0.564 | 0.508 | 0.730 |
| −0.431 | 1.008 | 0.706 | 0.960 | 0.714 | 0.716 | 0.643 |
| −0.908 | 0.700 | 0.797 | 1.011 | 1.014 | 0.742 | 0.730 |
| −1.386 | 1.124 | 0.835 | 1.158 | 0.883 | 0.728 | 0.774 |
| −1.863 | 1.148 | 1.026 | 0.898 | 0.926 | 0.835 | 1.142 |

| Conc (exp | | | | | | |
|---|---|---|---|---|---|---|
| 10) (nM) | WV-2124 | | WV-2126 | | WV-1422 IC50 = 0.29 nM | |
| 1.477 | 0.333 | 0.314 | 0.258 | 0.294 | 0.100 | 0.131 |
| 1.000 | 0.408 | 0.260 | 0.210 | 0.248 | 0.182 | 0.170 |
| 0.523 | 0.390 | 0.283 | 0.355 | 0.342 | 0.245 | 0.230 |
| 0.046 | 0.492 | 0.439 | 0.572 | 0.520 | 0.293 | 0.353 |
| −0.431 | 1.139 | 0.533 | 0.754 | 0.636 | 0.451 | 0.528 |
| −0.908 | 0.713 | 0.683 | 0.796 | 0.692 | 0.600 | 0.591 |
| −1.386 | 1.054 | 0.915 | 1.240 | 0.736 | 0.756 | 0.693 |
| −1.863 | 0.902 | 0.987 | 0.886 | 0.885 | | |

Table 57. Table 57 shows in vitro efficacy of different ASOs, which target APOC3. Oligonucleotides tested are: WV-2128 to WV-2139, and WV-1868. The oligonucleotides have different but overlapping base sequences.

TABLE 57
Activity of oligonucleotides.

| | | | |
|---|---|---|---|
| Conc (exp | | | |
| 10) (nM) | WV-2128 | WV-2129 | WV-2130 |

| | | | | | | |
|---|---|---|---|---|---|---|
| 1.477 | 0.224 | 0.270 | 0.214 | 0.239 | 0.231 | 0.227 |
| 1.000 | 0.246 | 0.210 | 0.201 | 0.194 | 0.385 | 0.303 |
| 0.523 | 0.405 | 0.480 | 0.318 | 0.326 | 0.540 | 0.447 |
| 0.046 | 0.550 | 0.564 | 0.547 | 0.534 | 0.751 | 0.862 |
| −0.431 | 0.968 | 0.865 | 0.742 | 0.835 | 0.910 | 1.249 |
| −0.908 | 0.955 | 0.948 | 0.909 | 0.812 | 1.003 | 1.267 |
| −1.386 | 0.907 | 1.074 | 0.964 | 0.990 | 1.007 | 1.031 |
| −1.863 | 1.007 | 1.026 | 1.118 | 1.016 | 1.114 | 0.965 |

| | | | |
|---|---|---|---|
| Conc (exp | | | |
| 10) (nM) | WV-2131 | WV-2132 | WV-2133 |

| | | | | | | |
|---|---|---|---|---|---|---|
| 1.477 | 0.329 | 0.339 | 0.300 | 0.279 | 0.433 | 0.287 |
| 1.000 | 0.430 | 0.329 | 0.367 | 0.404 | 0.434 | 0.355 |
| 0.523 | 0.652 | 1.132 | 0.727 | 0.918 | 0.450 | 0.565 |
| 0.046 | 0.836 | 0.829 | 1.075 | 0.958 | 0.747 | 0.687 |
| −0.431 | 1.251 | 1.341 | 0.990 | 1.231 | 1.004 | 1.313 |
| −0.908 | 0.982 | 0.990 | 0.914 | 1.438 | 0.989 | 0.917 |
| −1.386 | 1.011 | 1.033 | 1.049 | 1.095 | 1.100 | 1.013 |
| −1.863 | 1.058 | 0.894 | 0.973 | 1.010 | 0.937 | 0.927 |

| | | | |
|---|---|---|---|
| Conc (exp | | | |
| 10) (nM) | WV-2134 | WV-2135 | WV-2136 |

| | | | | | | |
|---|---|---|---|---|---|---|
| 1.477 | 0.379 | 0.389 | 0.346 | 0.358 | 0.455 | 0.483 |
| 1.000 | 0.264 | 0.225 | 0.261 | 0.301 | 0.324 | 0.300 |
| 0.523 | 0.402 | 0.372 | 0.355 | 0.408 | 0.337 | 0.342 |
| 0.046 | 0.574 | 0.573 | 0.564 | 0.582 | 0.444 | 0.442 |
| −0.431 | 0.756 | 0.480 | 0.874 | 0.975 | 0.647 | 0.957 |
| −0.908 | 0.887 | 0.817 | 0.923 | 1.070 | 0.764 | 0.833 |
| −1.386 | 1.038 | 1.405 | 1.033 | 0.909 | 0.941 | 0.889 |
| −1.863 | 1.127 | 0.933 | 0.880 | 1.062 | 1.008 | 0.959 |

| Conc (exp | | | | | | |
|---|---|---|---|---|---|---|
| 10) (nM) | WV-2137 | | WV-2139 | | WV-1868 IC50 = 0.62 nM | |
| 1.477 | 0.425 | 0.494 | 0.377 | 0.395 | 0.119 | 0.153 |
| 1.000 | 0.292 | 0.419 | 0.302 | 0.255 | 0.121 | 0.188 |
| 0.523 | 0.308 | 0.320 | 0.328 | 0.326 | 0.291 | 0.276 |
| 0.046 | 0.432 | 0.466 | 0.658 | 0.605 | 0.389 | 0.353 |
| −0.431 | 0.615 | 0.842 | 0.958 | 0.954 | 0.618 | 0.735 |
| −0.908 | 0.957 | 0.792 | 0.847 | 0.859 | 0.797 | 0.786 |
| −1.386 | 0.940 | 0.930 | 0.941 | 0.923 | 0.955 | 0.859 |
| −1.863 | 0.894 | 1.164 | 0.886 | 1.041 | | |

Table 58. Table 58 shows in vitro efficacy of different ASOs, which target APOC3. Oligonucleotides tested are: WV-2549 to WV-2554, WV-1422, and WV-1868. The oligonucleotides differ in overall length and in the length of the wings and core, and in stereochemistry.

TABLE 58
Activity of oligonucleotides.

| Conc (exp | | | | | | |
|---|---|---|---|---|---|---|
| 10) (nM) | WV-2549 (IC50 = 4.5 nM) | | WV-2550 (5.2 nM) | | WV-2551 (5.0 nM) | |
| 1.477 | 0.280 | 0.246 | 0.442 | 0.463 | 0.323 | 0.332 |
| 1.079 | 0.383 | 0.439 | 0.666 | 0.489 | 0.545 | 0.446 |
| 0.681 | 0.544 | 0.538 | 0.810 | 0.745 | 0.699 | 0.580 |
| 0.283 | 0.718 | 0.814 | 0.937 | 0.973 | 0.914 | 0.894 |
| −0.115 | 0.826 | 0.817 | 1.075 | 1.272 | 0.802 | 0.997 |
| −0.513 | 0.830 | 1.212 | 1.106 | 1.262 | 1.057 | 1.160 |
| −0.911 | 0.840 | 1.030 | 0.949 | 1.370 | 1.024 | 1.264 |
| −1.308 | 0.859 | 1.103 | 1.014 | 1.113 | 0.993 | 1.218 |
| −1.706 | 0.987 | 1.016 | 1.177 | 0.984 | 0.966 | 1.049 |
| −2.104 | 0.942 | 0.948 | 0.835 | 1.054 | 1.022 | 1.110 |
| −2.502 | 1.182 | 1.022 | 0.982 | 0.949 | 0.942 | 0.935 |

| Conc (exp | | | | | | |
|---|---|---|---|---|---|---|
| 10) (nM) | WV-2552 (3.3 nM) | | WV-2553 (4.2 nM) | | WV-2554 (2.9 nM) | |
| 1.477 | 0.306 | 0.366 | 0.245 | 0.248 | 0.288 | 0.212 |
| 1.079 | 0.496 | 0.464 | 0.441 | 0.423 | 0.369 | 0.216 |
| 0.681 | 0.504 | 0.492 | 0.527 | 0.650 | 0.583 | 0.433 |
| 0.283 | 1.017 | 0.768 | 0.720 | 0.889 | 0.769 | 0.703 |
| −0.115 | 0.811 | 0.865 | 0.809 | 1.058 | 0.800 | 0.788 |
| −0.513 | 1.030 | 0.920 | 1.307 | 1.148 | 1.015 | 1.047 |
| −0.911 | 1.027 | 1.132 | 1.021 | 1.215 | 1.064 | 1.243 |
| −1.308 | 0.787 | 1.188 | 0.928 | 0.943 | 0.955 | 0.951 |
| −1.706 | 1.123 | 1.146 | 1.002 | 1.199 | 1.009 | 0.938 |
| −2.104 | 0.892 | 1.032 | 1.005 | 0.883 | 0.976 | 0.909 |
| −2.502 | 1.054 | 0.906 | 0.977 | 0.955 | 0.986 | 0.994 |

| Conc (exp | | | | |
|---|---|---|---|---|
| 10) (nM) | WV-1868 (1.4 nM) | | WV-1422 (1 nM) | |
| 1.477 | 0.219 | 0.185 | 0.251 | 0.226 |
| 1.079 | 0.272 | 0.189 | 0.352 | 0.322 |
| 0.681 | 0.435 | 0.323 | 0.446 | 0.461 |
| 0.283 | 0.603 | 0.492 | 0.535 | 0.713 |
| −0.115 | 0.633 | 0.771 | 0.689 | 0.815 |
| −0.513 | 0.808 | 0.979 | 0.951 | 0.999 |
| −0.911 | 0.934 | 1.216 | 0.931 | 1.054 |
| −1.308 | 1.045 | 1.209 | 0.921 | 0.915 |
| −1.706 | 1.196 | 0.963 | 1.128 | 1.053 |
| −2.104 | 0.912 | 0.947 | 0.927 | 0.993 |
| −2.502 | 0.904 | 0.948 | 0.945 | 0.955 |

IC50 for various oligonucleotides is presented in parentheses after the oligonucleotide designation.

Table 59. Table 59 shows in vitro efficacy of different ASOs, which target APOC3. The oligonucleotides differ in overall length and in the length of the wings and core, and in stereochemistry.

TABLE 59
Activity of oligonucleotides.

| Conc (exp | | | | | | |
|---|---|---|---|---|---|---|
| 10) (nM) | WV-2549 1.76 nM | | WV-2550 3.1 nM | | WV-2551 1.8 nM | |
| 1.477 | 0.368 | 0.473 | 0.435 | 0.499 | 0.444 | 0.446 |
| 1.079 | 0.482 | 0.479 | 0.472 | 0.550 | 0.390 | 0.365 |
| 0.681 | 0.614 | 0.558 | 0.656 | 0.685 | 0.432 | 0.421 |
| 0.283 | 0.759 | 0.613 | 0.812 | 0.842 | 0.742 | 0.672 |
| −0.115 | 0.843 | 0.927 | 0.995 | 0.977 | 0.847 | 0.842 |
| −0.513 | 0.858 | 1.102 | 1.094 | 1.096 | 0.869 | 0.948 |
| −0.911 | 0.972 | 1.262 | 1.044 | 1.109 | 0.941 | 1.035 |
| −1.308 | 1.025 | 1.047 | 1.015 | 1.085 | 1.047 | 1.016 |
| −1.706 | 1.086 | 1.098 | 0.969 | 1.041 | 0.976 | 0.909 |
| −2.104 | 0.889 | 0.856 | 1.017 | 0.908 | 0.978 | 0.881 |

| Conc (exp | | | | | | |
|---|---|---|---|---|---|---|
| 10) (nM) | WV-2552 1.8 nM | | WV-2553 1.4 nM | | WV-2554 1.3 nM | |
| 1.477 | 0.643 | 0.438 | 0.303 | 0.303 | 0.227 | 0.262 |
| 1.079 | 0.497 | 0.376 | 0.349 | 0.238 | 0.215 | 0.199 |
| 0.681 | 0.519 | 0.454 | 0.441 | 0.394 | 0.326 | 0.295 |
| 0.283 | 0.755 | 0.694 | 0.627 | 0.509 | 0.543 | 0.477 |
| −0.115 | 1.022 | 0.868 | 0.807 | 0.610 | 0.710 | 0.641 |
| −0.513 | 1.093 | 0.958 | 0.962 | 0.811 | 0.836 | 0.847 |
| −0.911 | 0.989 | 1.142 | 0.997 | 0.964 | 0.856 | 0.986 |
| −1.308 | 1.037 | 1.109 | 1.045 | 1.006 | 1.021 | 0.935 |
| −1.706 | 1.031 | 0.873 | 0.922 | 0.809 | 1.019 | 0.821 |
| −2.104 | 0.932 | 0.795 | 1.033 | 0.836 | 0.960 | 0.869 |
| Conc (exp | | | | | | |
| 10) (nM) | WV-2677 1.5 nM | | WV-2678 1.8 nM | | WV-1422 1.1 nM | |
| 1.477 | 0.214 | 0.151 | 0.293 | 0.240 | 0.404 | 0.369 |
| 1.079 | 0.214 | 0.148 | 0.321 | 0.240 | 0.416 | 0.436 |
| 0.681 | 0.388 | 0.265 | 0.456 | 0.386 | 0.490 | 0.496 |
| 0.283 | 0.538 | 0.406 | 0.616 | 0.535 | 0.794 | 0.650 |
| −0.115 | 0.779 | 0.592 | 0.959 | 0.748 | 0.716 | 0.851 |
| −0.513 | 0.836 | 0.920 | 0.883 | 1.006 | 0.809 | 0.987 |
| −0.911 | 0.926 | 1.043 | 1.016 | 1.135 | 1.070 | 1.107 |
| −1.308 | 1.037 | 0.816 | 1.045 | 0.930 | 1.176 | 0.996 |
| −1.706 | 1.071 | 0.766 | 1.178 | 0.873 | | |
| −2.104 | 0.892 | 0.742 | 0.778 | 0.839 | | |

| | |
|---|---|
| Conc (exp | |
| 10) (nM) | WV-1868 1.65 nM |

| | | |
|---|---|---|
| 1.477 | 0.262 | 0.332 |
| 1.079 | 0.358 | 0.308 |
| 0.681 | 0.378 | 0.520 |
| 0.283 | 0.498 | 0.659 |
| −0.115 | 0.813 | 0.809 |
| −0.513 | 1.120 | 0.947 |
| −0.911 | 0.824 | 0.964 |
| −1.308 | | |
| −1.706 | | |
| −2.104 | | |

The IC50 is presented after the designation of the oligonucleotide.

Table 60. Tables 60A to H show the efficacy and stability of different ASOs, which target APOC3. Oligonucleotides tested are: Table 60A, WV-2551, WV-2553, WV-2554, WV-2678, WV-2677, and WV-1868. The oligonucleotides differ in the length of the wings and core, and in stereochemistry.

TABLE 60A
Activity of oligonucleotides.

| Conc (exp | | | | | | |
|---|---|---|---|---|---|---|
| 10) (nM) | WV-2549 1.76 nM | | WV-2550 3.1 nM | | WV-2551 1.8 nM | |
| 1.477 | 0.368 | 0.473 | 0.435 | 0.499 | 0.444 | 0.446 |
| 1.079 | 0.482 | 0.479 | 0.472 | 0.550 | 0.390 | 0.365 |
| 0.681 | 0.614 | 0.558 | 0.656 | 0.685 | 0.432 | 0.421 |
| 0.283 | 0.759 | 0.613 | 0.812 | 0.842 | 0.742 | 0.672 |
| −0.115 | 0.843 | 0.927 | 0.995 | 0.977 | 0.847 | 0.842 |
| −0.513 | 0.858 | 1.102 | 1.094 | 1.096 | 0.869 | 0.948 |
| −0.911 | 0.972 | 1.262 | 1.044 | 1.109 | 0.941 | 1.035 |
| −1.308 | 1.025 | 1.047 | 1.015 | 1.085 | 1.047 | 1.016 |
| −1.706 | 1.086 | 1.098 | 0.969 | 1.041 | 0.976 | 0.909 |
| −2.104 | 0.889 | 0.856 | 1.017 | 0.908 | 0.978 | 0.881 |

| Conc (exp | | | | | | |
|---|---|---|---|---|---|---|
| 10) (nM) | WV-2552 1.8 nM | | WV-2553 1.4 nM | | WV-2554 1.3 nM | |
| 1.477 | 0.643 | 0.438 | 0.303 | 0.303 | 0.227 | 0.262 |
| 1.079 | 0.497 | 0.376 | 0.349 | 0.238 | 0.215 | 0.199 |
| 0.681 | 0.519 | 0.454 | 0.441 | 0.394 | 0.326 | 0.295 |
| 0.283 | 0.755 | 0.694 | 0.627 | 0.509 | 0.543 | 0.477 |
| −0.115 | 1.022 | 0.868 | 0.807 | 0.610 | 0.710 | 0.641 |
| −0.513 | 1.093 | 0.958 | 0.962 | 0.811 | 0.836 | 0.847 |
| −0.911 | 0.989 | 1.142 | 0.997 | 0.964 | 0.856 | 0.986 |
| −1.308 | 1.037 | 1.109 | 1.045 | 1.006 | 1.021 | 0.935 |
| −1.706 | 1.031 | 0.873 | 0.922 | 0.809 | 1.019 | 0.821 |
| −2.104 | 0.932 | 0.795 | 1.033 | 0.836 | 0.960 | 0.869 |

| Conc (exp | | | | | | |
|---|---|---|---|---|---|---|
| 10) (nM) | WV-2677 1.5 nM | | WV-2678 1.8 nM | | WV-1422 All PS 1.1 nM | |
| 1.477 | 0.214 | 0.151 | 0.293 | 0.240 | 0.404 | 0.369 |
| 1.079 | 0.214 | 0.148 | 0.321 | 0.240 | 0.416 | 0.436 |
| 0.681 | 0.388 | 0.265 | 0.456 | 0.386 | 0.490 | 0.496 |
| 0.283 | 0.538 | 0.406 | 0.616 | 0.535 | 0.794 | 0.650 |
| −0.115 | 0.779 | 0.592 | 0.959 | 0.748 | 0.716 | 0.851 |
| −0.513 | 0.836 | 0.920 | 0.883 | 1.006 | 0.809 | 0.987 |
| −0.911 | 0.926 | 1.043 | 1.016 | 1.135 | 1.070 | 1.107 |
| −1.308 | 1.037 | 0.816 | 1.045 | 0.930 | 1.176 | 0.996 |
| −1.706 | 1.071 | 0.766 | 1.178 | 0.873 | | |
| −2.104 | 0.892 | 0.742 | 0.778 | 0.839 | | |

| Conc (exp | | |
|---|---|---|
| 10) (nM) | WV-1868 PS/PO 1.65 nM | |
| 1.477 | 0.262 | 0.332 |
| 1.079 | 0.358 | 0.308 |
| 0.681 | 0.378 | 0.520 |
| 0.283 | 0.498 | 0.659 |
| −0.115 | 0.813 | 0.809 |
| −0.513 | 1.120 | 0.947 |
| −0.911 | 0.824 | 0.964 |
| −1.308 | | |
| −1.706 | | |
| −2.104 | | |

The IC50 is presented after the designation of the oligonucleotide.

As shown in Table 60B, WV-1878, WV-1868, and WV-1871 were tested in vitro. The oligonucleotides differ in the length of the wings and core, and in base sequence.

TABLE 60B
Activity of oligonucleotides.

| Oligonucleotide | IC50 (nM) |
|---|---|
| WV-1878 | 2.1 |
| WV-1868 | 0.76 |
| WV-1871 | 2.7 |

As shown in Table 60C, WV-2141 and WV-3968 were tested in vitro.

TABLE 60C
Activity of oligonucleotides.

| | | |
|---|---|---|
| Conc (uM) | WV-2141 | WV-3968 |

| | | | | |
|---|---|---|---|---|
| 0 | 87.9 | 93.1 | 87.9 | 93.1 |
| 1 | 97.9 | 80.6 | 32.7 | 45.6 |
| 5 | 56.6 | 67.4 | 42.6 | 29.6 |
| 10 | 47.7 | 68.1 | 41.3 | 59.5 |

Numbers indicate % APOC3 mRNA remaining (AOC3/HPRT1). The 5′ end of WV-3968 comprises a Mod001 moiety (as defined in the legend to Table 1A) and a linker L001.

As shown in Table 60D, WV-2647, WV-2647, WV-2552, WV-2551, WV-2549, WV-2550, WV-2554, WV-2553, WV-2646, WV-2645, WV-1422, WV-2677, WV-723, WV-2678, WV-1868, WV-819, and WV-2644 were tested for stability in vitro.

TABLE 60D
Stability of oligonucleotides.

| Days | WV723-1 | WV723-2 | WV723-3 |
|---|---|---|---|
| 0 | 100 | 100 | 100 |
| 0.333 | 49.09815078 | 50.75705 | 46.66902 |
| 0.666 | 42.56045519 | 44.04259 | 40.76915 |
| 1 | 39.58463727 | 38.01295 | 36.69628 |
| 2 | 28.35277383 | 26.27468 | 27.87928 |
| 3 | 19.93172119 | 23.00572 | 22.19643 |
| 4 | 16.17638691 | 15.66529 | 16.07449 |
| 5 | 14.685633 | 13.81414 | 12.93025 |

| Days | WV819-1 | WV819-2 | WV819-3 |
|---|---|---|---|
| 0 | 100 | 100 | 100 |
| 0.333 | 43.51021872 | 46.65438 | 44.26357 |
| 0.666 | 38.36052349 | 39.37896 | 36.76867 |
| 1 | 32.56095375 | 37.27138 | 30.99551 |
| 2 | 21.00215131 | 23.34067 | 22.01958 |
| 3 | 15.66869846 | 16.55507 | 16.09547 |
| 4 | 13.55324489 | 18.68961 | 12.27254 |
| 5 | 10.54141269 | 12.78929 | 9.277846 |

| Days | WV1422-1 | WV1422-2 | WV1422-3 |
|---|---|---|---|
| 0 | 100 | 100 | 100 |
| 0.333 | 70.51666845 | 67.40684 | 62.96961 |
| 0.666 | 59.69557294 | 53.65779 | 52.61469 |
| 1 | 50.13399078 | 49.47022 | 46.16292 |
| 2 | 34.19980705 | 32.08112 | 31.21106 |
| 3 | 25.71551077 | 23.65526 | 22.862 |
| 4 | 22.55332833 | 20.25856 | 19.36473 |
| 5 | 17.25801265 | 14.45374 | 15.72116 |
| Days | WV1868-1 | WV1868-2 | WV1422-3 |
| 0 | 100 | 100 | 100 |
| 0.333 | 67.74587087 | 69.0377 | 65.87368 |
| 0.666 | 51.09797297 | 51.80944 | 51.05068 |
| 1 | 40.01814314 | 40.39831 | 40.40356 |
| 2 | 22.76338839 | 22.60573 | 20.85249 |
| 3 | 14.17042042 | 14.52475 | 13.57829 |
| 4 | 10.12575075 | 8.851996 | 9.154677 |
| 5 | 6.969469469 | 6.791561 | 6.716017 |
| Days | WV2549-1 | WV2549-2 | WV2549-3 |
| 0 | 100 | 100 | 100 |
| 0.333 | 93.91357898 | 92.79193 | 93.26989 |
| 0.666 | 92.45612234 | 88.80706 | 91.78848 |
| 1 | 86.59550446 | 86.70366 | 85.23006 |
| 2 | 74.17119984 | 76.55485 | 74.57078 |
| 3 | 67.43816073 | 67.03657 | 66.13362 |
| 4 | 59.98665709 | 57.77554 | 56.80859 |
| 5 | 48.6246536 | 46.3657 | 47.62092 |
| Days | WV2550-1 | WV2550-2 | WV2550-3 |
| 0 | 100 | 100 | 100 |
| 0.333 | 95.48114142 | 93.89125 | 89.39343 |
| 0.666 | 93.0236745 | 86.26168 | 86.79865 |
| 1 | 85.48024125 | 81.28292 | 82.89806 |
| 2 | 72.32874246 | 69.37978 | 67.81803 |
| 3 | 57.33189306 | 55.57349 | 55.64448 |
| 4 | 50.21154019 | 47.66355 | 47.21146 |
| 5 | 38.4463048 | 36.25319 | 38.91323 |
| Days | WV2551-1 | WV2551-2 | WV2551-3 |
| 0 | 100 | 100 | 100 |
| 0.333 | 92.39859107 | 94.76026 | 95.3919 |
| 0.666 | 90.27383252 | 92.22383 | 88.72739 |
| 1 | 87.85365299 | 88.97541 | 86.35874 |
| 2 | 78.17293489 | 79.9644 | 73.86951 |
| 3 | 66.67424156 | 66.80387 | 68.57235 |
| 4 | 60.27724122 | 59.30582 | 58.3441 |
| 5 | 49.80115896 | 49.6162 | 50.49526 |
| Days | WV2552-1 | WV2552-2 | WV2552-3 |
| 0 | 100 | 100 | 100 |
| 0.333 | 90.98589239 | 97.80328 | 99.99086 |
| 0.666 | 88.69750656 | 96.01004 | 96.23538 |
| 1 | 84.4488189 | 88.01219 | 95.87902 |
| 2 | 70.97276903 | 80.59715 | 84.91411 |
| 3 | 63.32841207 | 64.88837 | 74.57968 |
| 4 | 57.58694226 | 60.21698 | 64.41886 |
| 5 | 46.05479003 | 53.39371 | 55.2723 |
| Days | WV2553-1 | WV2553-2 | WV2553-3 |
| 0 | 100 | 100 | 100 |
| 0.333 | 92.2305438 | 97.18409 | 90.91079 |
| 0.666 | 90.83034842 | 84.13727 | 84.48244 |
| 1 | 77.52523608 | 77.0109 | 80.18029 |
| 2 | 65.01465321 | 65.03528 | 62.86602 |
| 3 | 55.49332465 | 53.52149 | 54.65962 |
| 4 | 45.88733312 | 42.96985 | 41.46099 |
| 5 | 33.82611527 | 34.58627 | 33.76438 |
| Days | WV2554-1 | WV2554-2 | WV2554-3 |
| 0 | 100 | 100 | 100 |
| 0.333 | 94.79862111 | 97.72837 | 95.30518 |
| 0.666 | 98.32002444 | 90.65912 | 90.90406 |
| 1 | 138.9448881 | 85.30018 | 83.4213 |
| 2 | 66.28267225 | 69.27597 | 69.07721 |
| 3 | 55.59191866 | 56.75569 | 56.33779 |
| 4 | 45.70406249 | 44.59501 | 44.68801 |
| 5 | 32.78352315 | 34.10078 | 34.8472 |
| Days | WV2644-1 | WV2644-2 | WV2644-3 |
| 0 | 100 | 100 | 100 |
| 0.333 | 0.059184568 | 0.088216 | 0.068567 |
| 0.666 | 0.026304253 | 0.050724 | 0.063293 |
| 1 | 0.026304253 | 0.02867 | 0.031646 |
| Days | WV2645-1 | WV2645-2 | WV2645-3 |
| 0 | 100 | 100 | 100 |
| 0.333 | 73.70322969 | 80.76862 | 79.76236 |
| 0.666 | 68.32793797 | 72.95462 | 70.16434 |
| 1 | 59.60249944 | 61.37744 | 59.47844 |
| 2 | 38.09380411 | 42.64941 | 40.05864 |
| 3 | 24.79861477 | 25.3116 | 25.41471 |
| 4 | 15.38056162 | 16.08341 | 15.97871 |
| 5 | 10.11066777 | 10.11505 | 10.07638 |
| Days | WV2646-1 | WV2646-2 | WV2646-3 |
| 0 | 100 | 100 | 100 |
| 0.333 | 70.8897207 | 71.01162 | 68.12728 |
| 0.666 | 64.49035416 | 60.79626 | 59.61305 |
| 1 | 53.49841635 | 53.76877 | 49.8854 |
| 2 | 32.93982148 | 33.63559 | 34.01645 |
| 3 | 23.74748056 | 23.42732 | 23.40569 |
| 4 | 15.99481716 | 16.70445 | 16.24646 |
| 5 | 17.77281889 | 11.52593 | 12.8219 |
| Days | WV2647-1 | WV2647-2 | WV2647-3 |
| 0 | 100 | 100 | 100 |
| 0.333 | 91.14885115 | 91.36755 | 88.88889 |
| 0.666 | 94.40559441 | 87.56281 | 88.5921 |
| 1 | 88.15184815 | 83.3094 | 85.51289 |
| 2 | 84.31568432 | 78.24838 | 80.50454 |
| 3 | 79.000999 | 71.30294 | 74.23484 |
| 4 | 74.64535465 | 67.13927 | 69.24504 |
| 5 | 72.76723277 | 63.87294 | 65.7021 |
| Days | WV2677-2 | WV2677-2 | WV2677-3 |
| 0 | 100 | 100 | 100 |
| 0.333 | 74.16923604 | 68.33221 | 79.12078 |
| 0.666 | 56.90733128 | 54.40283 | 60.87654 |
| 1 | 46.88677629 | 45.06097 | 51.94168 |
| 2 | 31.20075368 | 28.98677 | 36.2923 |
| 3 | 24.36193902 | 22.31004 | 26.17147 |
| 4 | 17.72439192 | 17.54776 | 20.2684 |
| 5 | 15.74169236 | 14.89057 | 15.422 |
| Days | WV2678-1 | WV2678-2 | WV2678-3 |
| 0 | 100 | 100 | 100 |
| 0.333 | 66.0880372 | 64.71483 | 59.23107 |
| 0.666 | 45.11706222 | 45.80369 | 46.8807 |
| 1 | 36.52581783 | 38.74748 | 36.68648 |
| 2 | 23.66500962 | 24.55282 | 25.08918 |
| 3 | 17.4390635 | 17.61997 | 17.87951 |
| 4 | 12.93296985 | 12.52519 | 13.1193 |
| 5 | 9.878127004 | 10.02508 | 10.09116 |

The oligonucleotides were incubated in rat liver homogenate at 20 uM for 0 d (0 days), 8h (8 hours), 16h, 1 d, 2 d, 3 d, 4 d, or 5 d and the remained full length oligonucleotides were plotted in percentage. Numbers indicate percentage of full-length oligonucleotide remaining. For example, in column 2, row 2, 100 indicates 100% full-length oligonucleotide remaining. Data from replicate experiments (e.g., −1, −2, etc.) are shown.

As shown in Table 60E, WV-2725, WV-2726, WV-2727, WV-2722, WV-2723, and WV-2724 were tested for stability in vivo.

TABLE 60E
Stability of oligonucleotides in liver.

| Days | PBS | WV-2725 | WV-2726 | WV-2727 | WV-2722 | WV-2723 | WV-2724 |
|---|---|---|---|---|---|---|---|
| 1 | 2 | 36 | 75 | 51 | 35 | 49 | 39 |
| 5 | | 26 | 43 | 33 | 19 | 26 | 15 |
| 15 | 2 | 7 | 13 | 11 | 4 | 7 | 4 |

Numbers indicate ug/g of ASO in liver at a number of days after injection into test animals. Numbers are approximate and error bars are not shown. Approximately 75 ug/g represents about 90% of the injected dose, and about 13 ug/g represents about 16% of the injected dose. The oligonucleotides differ in stereochemistry pattern and 2′-modifications.

As shown in Table 60F and 60G, WV-3968 and WV-6003 were tested. The 5′ end of WV-3968 and that of WV-6003 comprise a Mod001 moiety (as defined in the legend to Table 1A) and a linker L001:

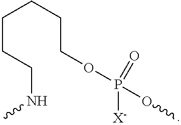

Data represent relative hAPOC3 protein levels (relative to PBS)

TABLE 60F
Part I. Activity of oligonucleotides.

| | Stereorandom | | | | |
|---|---|---|---|---|---|
| Day | WV-3968 | | | | |
| 8 | 0.062 | 0.013 | 0.018 | 0.016 | 0.035 |
| 15 | 0.017 | 0.009 | 0.01 | 0.012 | 0.014 |
| 22 | 0.039 | 0.027 | 0.019 | 0.023 | 0.023 |
| 29 | 0.153 | 0.043 | 0.021 | 0.06 | 0.072 |
| 35 | 0.096 | 0.172 | 0.088 | 0.172 | 0.09 |
| 42 | 0.304 | 0.228 | 0.209 | 0.317 | 0.402 |
| 50 | 0.396 | 0.459 | 0.02 | 0.351 | 0.165 |
| 57 | 1.038 | 1.223 | 0.623 | 0.792 | 0.654 |
| 64 | 1.011 | 0.92 | 0.458 | 0.621 | 0.551 |
| | Stereopure | | | | |
| Day | WV-6003 | | | | |
| 8 | 0.016 | 0.043 | 0.032 | 0.039 | 0.016 |
| 15 | 0.015 | 0.016 | 0.021 | 0.024 | 0.021 |
| 22 | 0.033 | 0.039 | 0.051 | 0.047 | 0.027 |
| 29 | 0.023 | 0.031 | 0.046 | 0.096 | 0.048 |
| 35 | 0.266 | 0.159 | 0.067 | 0.176 | 0.239 |
| 42 | 0.104 | 0.084 | 0.069 | 0.084 | 0.067 |
| 50 | 0.072 | 0.193 | 0.065 | 0.133 | 0.064 |
| 57 | 0.334 | 0.782 | 0.381 | 0.519 | 0.21 |
| 64 | 0.397 | 0.754 | 0.543 | 0.699 | 0.239 |

TABLE 60F
Part II. Activity of oligonucleotides.

| | Stereorandom | | | | |
|---|---|---|---|---|---|
| Day | WV-3968 | | | | |
| 8 | 0.062 | 0.013 | 0.018 | 0.016 | 0.035 |
| 15 | 0.017 | 0.009 | 0.01 | 0.012 | 0.014 |
| 22 | 0.039 | 0.027 | 0.019 | 0.023 | 0.023 |
| 29 | 0.153 | 0.043 | 0.021 | 0.06 | 0.072 |
| 35 | 0.096 | 0.172 | 0.088 | 0.172 | 0.09 |
| 50 | 0.396 | 0.459 | 0.02 | 0.351 | 0.165 |
| 64 | 0.873 | 1.34 | 0.407 | 0.613 | 0.492 |
| 78 | 0.904 | 1.971 | 1.292 | 1.611 | 0.638 |
| | Stereopure | | | | |
| Day | WV-6003 | | | | |
| 8 | 0.016 | 0.043 | 0.032 | 0.039 | 0.016 |
| 15 | 0.015 | 0.016 | 0.021 | 0.024 | 0.021 |
| 22 | 0.033 | 0.039 | 0.051 | 0.047 | 0.027 |
| 29 | 0.023 | 0.031 | 0.046 | 0.096 | 0.048 |
| 35 | 0.266 | 0.159 | 0.067 | 0.176 | 0.239 |
| 50 | 0.072 | 0.193 | 0.065 | 0.133 | 0.064 |
| 64 | 0.43 | 0.591 | 0.401 | 0.235 | 0.12 |
| 78 | 0.399 | 0.669 | 0.557 | 1.358 | 0.442 |

Data represent relative hAPOC3 protein levels (relative to PBS).
In some experiments, compounds were constructed which comprise an APOC3 oligonucleotide (WV-7107) conjugated to a mono-, bis- or tri-antennary GalNAc (also designated Ref. GalNAc or Reference GalNAc) or PFE ligand (also described as PFE ASPGR ligand, PFE GalNAc, bridged bicyclic ketal or bicyclic ligand).
Oligonucleotides tested are listed in Table 60G, Part I

TABLE 60G
Part I. List of oligonucleotides

| | | | | Example |
|---|---|---|---|---|
| | | Alternative | Example | describing |
| | | designation | describing | example |
| | | of ligand and | example | synthesis of |
| Oligo- | | linker (L001 | synthesis | oligonucleotide |
| nucleotide | Ligand | is a linker) | of ligand | with ligand |
| WV-8877 | None | — | — | — |
| WV-7107 | None | — | — | 37A |
| WV-6558 | Ref. GalNAc | Mod001L001 | 38 | 37A, 37B |
| | Tri-antennary | | (protected | |
| | | | version) | |
| WV-9542 | PFE ligand | Mod083L001 | 31, 40 | 37C |
| | Tri-antennary | | | |
| WV-9543 | Ref. GalNAc | Mod079L001 | 35 | 37D |
| | Bis-antennary | | (protected | |
| | | | version) | |
| WV-9544 | PFE ligand | Mod080L001 | 32, 33 | 37E |
| | Bis-antennary | | | |
| WV-9545 | Ref. GalNAc | Mod081L001 | 36 | 37F |
| | Mono- | | (protected | |
| | antennary | | version) | |
| WV-9546 | PFE ligand | Mod082L001 | 34 | 37G |
| | Mono- | | | |
| | antennary | | | |

Ref GalNAc Tri-antennary is also designated Tri-GalNAc; PFE ligand Tri-antennary is also designated Tri-PFE ligand; Ref. GalNAc Bis-antennary is also designated Bis-GalNAc; PFE ligand Bis-antennary is also designated Bis-PFE ligand; Ref. GalNAc Mono-antennary is also designated Mono-GalNAc; and PFE ligand Mono-antennary is also designated Mono-PFE ligand. The structures of Mod001, Mod079, Mod080, Mod081, Mod082, Mod083 and L001 are provided in the legend to Table 1A and in other texts herein. Ligands are also described in Example 27. Mod083 is also described in Example 4A and 4B. The GalNAc structures in Examples 29, 35, and 36 represent the protected versions, as they comprise —OAc (—O-acetate groups). In construction of the listed oligonucleotides, the Ac groups are removed during de-protection following conjugation of the compound to the oligonucleotide. De-protection is performed, for example, with concentrated ammonium hydroxide, e.g., as described in Example 37B. In the de-protected versions of these structures, —OAc is replaced by —OH.
WV-8877 (negative control) targets a different gene, which is not APOC3 or PNPLA3.
The APOC3 oligonucleotide WV-7107, conjugated with GalNAc or PFE ligand at different valencies (mono, bis or triantennary) and the negative control were separately administered to Tg (transgenic) mice harboring the human APOC3 transgene (B6.Cg-Tg(APOC3)2Bres/J) on day 1, and APOC3 knockdown was monitored by serum hAPOC3 protein reduction.

TABLE 60G
Part II. Activity of oligonucleotides

| Day | 0 | 8 | 15 | 22 | 29 | 36 | 43 | 50 |
|---|---|---|---|---|---|---|---|---|
| PBS | 1.52 | 0.95 | 1.50 | 0.56 | 0.96 | 1.07 | 1.57 | 1.74 |
| | 0.59 | 0.73 | 0.74 | 0.87 | 0.90 | 0.90 | 0.73 | 0.71 |
| | 1.21 | 0.99 | 1.10 | 1.34 | 0.89 | 0.82 | 0.62 | 0.78 |
| | 0.67 | 1.14 | 0.89 | 0.99 | 0.89 | 0.86 | 0.95 | 0.86 |
| | 1.01 | 1.20 | 0.76 | 1.24 | 1.35 | 1.36 | 1.13 | 0.91 |
| WV-8877 | 1.56 | 1.24 | 1.67 | 1.59 | 2.37 | 1.56 | 1.47 | 2.27 |
| | 0.78 | 0.73 | 0.85 | 0.80 | 1.15 | 0.61 | 0.75 | 1.19 |
| | 1.08 | 0.81 | 1.42 | | 1.84 | 1.21 | 1.73 | 3.05 |
| | 0.71 | 1.21 | 0.74 | 0.62 | 1.02 | 1.07 | 0.95 | 1.48 |
| | 1.28 | 1.21 | 0.60 | 0.80 | 1.13 | 1.50 | 0.86 | |
| WV-6558 | 2.74 | 0.06 | 0.05 | 0.06 | 0.11 | 0.38 | 0.69 | 1.43 |
| | 1.15 | 0.17 | 0.05 | 0.04 | 0.09 | 0.27 | 0.01 | 0.81 |
| | 0.38 | 0.04 | 0.05 | 0.10 | 0.18 | 0.45 | 0.53 | 1.07 |
| | 0.44 | | | | | | | |
| | 0.41 | 0.04 | 0.04 | 0.08 | 0.09 | 0.11 | 0.13 | 0.22 |
| WV-9542 | 1.10 | 0.23 | 0.05 | 0.07 | 0.13 | 0.23 | 0.32 | 0.78 |
| | 0.71 | 0.03 | 0.02 | 0.04 | 0.06 | 0.09 | 0.20 | 0.28 |
| | 0.59 | 0.05 | 0.04 | 0.08 | 0.16 | 0.72 | 0.90 | 0.80 |
| | 0.32 | 0.03 | 0.02 | 0.04 | 0.09 | 0.37 | 0.54 | 0.55 |
| | 0.40 | 0.03 | 0.03 | 0.06 | 0.21 | 0.39 | 0.49 | 0.58 |
| WV-9543 | 0.48 | 0.03 | 0.05 | 0.09 | 0.08 | 0.21 | 0.27 | 0.49 |
| | 1.19 | 0.06 | 0.06 | 0.09 | 0.06 | 0.09 | 0.57 | 0.96 |
| | 0.79 | 0.05 | 0.04 | 0.17 | 0.06 | 0.15 | 0.42 | 0.80 |
| | 0.79 | 0.09 | 0.03 | 0.28 | 0.20 | 0.17 | 0.28 | 0.59 |
| | 0.48 | 0.04 | 0.02 | 0.08 | 0.06 | 0.12 | 0.17 | 0.32 |
| WV-9544 | 0.91 | | 0.04 | 0.06 | 0.06 | 0.19 | 0.26 | 0.67 |
| | 0.94 | 0.10 | 0.03 | 0.08 | 0.09 | 0.15 | 0.34 | 0.76 |
| | 1.72 | 0.19 | 0.04 | 0.07 | 0.09 | 0.25 | 0.60 | 0.83 |
| | 1.92 | 0.28 | 0.07 | 0.10 | 0.11 | 0.13 | 0.26 | 0.56 |
| | 0.81 | 0.04 | 0.05 | 0.11 | 0.12 | 0.20 | 0.32 | 0.73 |
| WV-9545 | 0.49 | 0.03 | 0.07 | 0.16 | 0.21 | 0.32 | 0.66 | 0.60 |
| | 1.14 | 0.22 | 0.04 | 0.10 | 0.15 | 0.58 | 0.76 | 0.97 |
| | 0.58 | 0.03 | 0.04 | 0.15 | 0.27 | 0.67 | 1.16 | 0.97 |
| | 0.64 | 0.03 | 0.04 | 0.19 | 0.42 | 0.98 | 1.38 | 0.96 |
| | 0.60 | 0.05 | 0.03 | 0.08 | | | | |
| WV-9546 | 3.33 | 0.20 | 0.06 | 0.27 | 0.24 | 0.49 | 1.13 | 1.31 |
| | 1.03 | 0.11 | 0.04 | 0.09 | 0.14 | 0.46 | 0.55 | 0.68 |
| | 1.20 | 0.28 | 0.12 | 0.20 | 0.31 | 0.95 | 1.75 | 1.39 |
| | 0.71 | 0.15 | 0.04 | 0.19 | 0.39 | 0.26 | 0.75 | 0.36 |
| | 0.18 | 0.04 | 0.02 | 0.20 | 0.28 | 0.21 | 0.56 | 0.56 |

In this experiment, all oligonucleotides were administered to animals at a 3 mg/kg single dose (s.c.) at day 1. In addition, WV-6558 and WV-9542 were also administered to animals at a 1 mg/kg single dose (s.c.) at day 1. Serum was collected at days 0, 8, 15, 22, 29, 36, 43, and 50. Each group contained 5 animals. PBS and WV-8877 (which targets a gene which is not APOC3) were negative controls. Numbers indicate relative APOC3 protein level, wherein 1.00 represents 100% relative to PBS. In various in vivo studies, including this one, tested animals were transgenic mice expressing the human APOC3 gene.

TABLE 60H
Part I. Oligonucleotide accumulation in the liver

| PBS | WV-6558 | WV-9542 | WV-9543 | WV-9544 | WV-9545 | WV-9546 |
|---|---|---|---|---|---|---|
| 0 | 2.95 | 1.73 | 3.52 | 3.82 | 2.02 | 4.27 |
| 0 | 2.46 | 1.69 | 2.49 | 4.19 | 1.99 | 1.37 |
| 0 | 2.48 | 0.45 | 1.14 | 2.74 | 1.30 | 1.29 |
| 0 | 1.85 | 1.09 | 2.12 | 2.26 | 1.14 | 1.25 |
| 0 | 1.79 | 1.43 | 4.26 | 1.88 | 1.07 | 0.82 |

Oligonucleotide accumulation in the liver was also analyzed after a single 3 mg/kg dose, 30 min. Numbers indicate pg of oligonucleotide/g of tissue. Tested animals were transgenic mice expressing the human APOC3 gene.
In the same experiment: Oligonucleotide accumulation in the liver was also analyzed for WV-6558 and WV-9542 after a single 1 mg/kg dose, 30 min. Numbers indicate pg of oligonucleotide/g of tissue.

| | WV-6558 | WV-9542 |
|---|---|---|
| PBS | 1 mpk | 1 mpk |
| 0 | 1.92 | 0.46 |
| 0 | 1.77 | 1.08 |
| 0 | 1.43 | 0.56 |
| 0 | 0.68 | 0.30 |
| 0 | 0.18 | 0.67 |

TABLE 60H
Part II. Oligonucleotide accumulation in the liver

| PBS | WV-6558 | WV-9542 | WV-9543 | WV-9544 | WV-9545 | WV-9546 |
|---|---|---|---|---|---|---|
| 0 | 3.30 | 2.93 | 6.83 | 4.56 | 3.55 | 3.83 |
| 0 | 3.49 | 2.20 | 6.56 | 4.45 | 2.23 | 4.05 |
| 0 | 3.18 | 1.34 | 4.58 | 2.72 | 1.94 | 2.28 |
| 0 | 2.41 | 1.61 | 3.87 | 2.31 | 3.03 | 2.12 |
| 0 | 1.43 | 2.90 | 4.10 | 2.36 | 1.85 | 3.50 |

Oligonucleotide accumulation in the liver was also analyzed after a single 3 mg/kg dose, 8 days. Numbers indicate μg of oligonucleotide/g of tissue. Tested animals were transgenic mice expressing the human APOC3 gene.
In the same experiment: Oligonucleotide accumulation in the liver was also analyzed for WV-6558 and WV-9542 after a single 1 mg/kg (1 mpk) dose, 8 days. Numbers indicate ug of oligonucleotide/g of tissue.

| | WV-6558 | WV-9542 |
|---|---|---|
| PBS | 1 mpk | 1 mpk |
| 0 | 0.72 | 1.08 |
| 0 | 0.74 | 1.20 |
| 0 | 0.60 | 0.75 |
| 0 | 0.55 | 0.57 |
| 0 | 0.63 | 0.63 |

As shown in Table 601, animals were dosed subcutaneously with 10 mpk (mg/kg animal weight) of oligonucleotide on days 1 and 5, and samples were obtained for testing on days −2, 5, 8 and 28. Level of serum APOC3 is shown.

TABLE 60I
Activity of oligonucleotides

| | −2 | 5 | 8 | 14 | 21 | 28 |
|---|---|---|---|---|---|---|
| PBS | 0.85 | 0.77 | 0.77 | 0.87 | 0.70 | 0.59 |
| | 0.82 | 0.80 | 0.85 | 0.78 | 0.62 | 0.67 |
| | 1.21 | 1.23 | 1.22 | 1.22 | 1.32 | 1.33 |
| | 1.12 | 1.20 | 1.16 | 1.13 | 1.36 | 1.41 |
| WV-2722 | 0.89 | 0.71 | 0.53 | 0.70 | 0.86 | 0.95 |
| | 0.95 | 0.52 | 0.27 | 0.64 | 0.72 | 0.79 |
| | 1.12 | 0.89 | 0.44 | 0.84 | 1.03 | 1.36 |
| | 1.10 | 1.08 | 0.71 | 0.82 | 1.06 | 1.46 |
| WV-4204 | 0.78 | 0.75 | 0.42 | 0.61 | 0.70 | 0.68 |
| | 0.81 | 0.70 | 0.35 | 0.43 | 0.50 | 0.52 |
| | 1.13 | 1.10 | 0.73 | 0.71 | 0.63 | 1.21 |
| | 1.09 | 0.87 | 0.36 | 0.10 | 0.27 | 0.65 |
| WV-4205 | 0.94 | 0.78 | 0.73 | 0.28 | 0.86 | 0.90 |
| | 0.64 | 0.45 | 0.26 | 0.20 | 0.73 | 0.71 |
| | 1.07 | 0.74 | 0.35 | 0.16 | 0.74 | 0.96 |
| | 1.14 | 1.07 | 0.93 | 0.42 | 1.37 | 1.42 |
| WV-4206 | 1.10 | 1.10 | 1.08 | 0.57 | 1.22 | 1.21 |
| | 1.06 | 0.98 | 0.88 | 0.39 | 0.84 | 1.06 |
| | 0.72 | 0.63 | 0.43 | 0.19 | 0.56 | 0.45 |
| | 0.85 | 0.81 | 0.86 | 0.37 | 0.87 | 0.93 |
| WV-4207 | 0.79 | 0.92 | 0.76 | 0.31 | 0.71 | 0.68 |
| | 0.77 | 0.96 | 0.89 | 0.35 | 0.94 | 0.91 |
| | 1.01 | 1.01 | 0.99 | 0.78 | 1.17 | 1.40 |
| | 1.05 | 0.93 | 1.03 | 0.65 | 1.33 | 1.34 |
| WV-4208 | 0.87 | 0.96 | 0.78 | 0.83 | 0.80 | 0.61 |
| | 0.82 | 0.71 | 0.45 | 0.31 | 0.41 | 0.52 |
| | 1.08 | 0.89 | 0.64 | 0.64 | 0.70 | |
| | 1.07 | 1.05 | 0.85 | 0.92 | 1.08 | 1.17 |
| WV-4209 | 1.07 | 0.91 | 0.92 | 0.96 | 1.13 | 1.18 |
| | 1.00 | 0.71 | 0.82 | 0.86 | 1.02 | 1.11 |
| | 1.05 | 1.12 | 0.94 | 1.03 | 1.18 | 1.28 |
| | 1.01 | 1.08 | 0.94 | 0.99 | 1.28 | 1.42 |
| WV-4210 | 0.95 | 0.73 | 0.74 | 0.72 | 0.88 | 0.80 |
| | 0.88 | 0.71 | 0.54 | 0.50 | 0.67 | 0.66 |
| | 1.00 | 0.99 | 0.95 | 0.98 | 1.06 | 1.13 |
| | 1.13 | 1.10 | 0.95 | 1.01 | 0.96 | 1.32 |
| WV-4211 | 1.01 | 0.81 | 0.43 | 0.15 | | |
| | 0.70 | 0.78 | 0.46 | 0.17 | 0.63 | 0.55 |
| | 1.08 | 1.06 | 0.89 | 0.33 | 1.10 | 1.23 |
| | 1.02 | 1.11 | 0.89 | 0.41 | 0.94 | 1.31 |
| WV-4212 | 1.01 | 0.95 | 0.93 | 0.39 | | |
| | 0.95 | 0.90 | 0.84 | 0.36 | 0.94 | 0.95 |
| | 0.91 | 0.97 | 0.88 | 0.35 | 1.01 | 0.96 |
| | 1.07 | 1.06 | 0.92 | 0.41 | 0.87 | 0.96 |
| WV-4213 | 1.00 | 1.00 | 0.91 | 0.49 | 1.11 | 1.14 |
| | 0.82 | 0.77 | 0.68 | 0.26 | 0.74 | 0.67 |
| | 1.11 | 1.03 | 1.00 | 0.48 | 1.03 | 0.92 |
| | 1.07 | 1.11 | 1.03 | 0.56 | 1.36 | 1.40 |
| WV-4214 | 0.36 | 0.23 | 0.19 | 0.22 | 0.82 | 0.82 |
| | 0.27 | 0.17 | 0.14 | 0.20 | 0.52 | 0.58 |
| | 0.71 | 0.52 | 0.39 | 0.52 | 1.41 | 1.33 |
| | 0.71 | 0.32 | 0.07 | 0.14 | 0.93 | 1.17 |
| WV-4215 | 0.58 | 0.45 | 0.47 | 0.62 | 1.38 | 1.21 |
| | 0.39 | 0.39 | 0.34 | 0.55 | 1.31 | 1.36 |
| | 0.29 | 0.12 | 0.11 | 0.15 | 0.51 | 0.54 |
| | 0.22 | 0.19 | 0.11 | 0.16 | 0.57 | 0.70 |
| WV-4216 | 0.33 | 0.11 | 0.09 | 0.23 | 0.81 | 0.71 |
| | 0.38 | 0.12 | 0.10 | 0.17 | 0.67 | 1.18 |
| | 0.68 | 0.45 | 0.34 | 0.42 | 1.41 | 1.41 |
| | 0.56 | 0.25 | 0.12 | 0.24 | 1.17 | 1.27 |

As shown in Table 60J, animals were dosed subcutaneously with 10 mpk (mg/kg animal weight) of oligonucleotide on days 1 and 5, and samples were obtained for testing on days −2, 1, 5, 8, 14, 21, and 28. Level of APOC3 protein and TG (triglycerides) is shown.

TABLE 60J
Activity of oligonucleotides

| | Day | −2 | 5 | 8 | 14 |
|---|---|---|---|---|---|
| PBS | ApoC3 | 0.89 | 0.74 | 0.71 | 0.63 |
| | | 0.75 | 0.72 | 0.47 | 0.60 |
| | | 1.26 | 1.20 | 1.07 | 1.38 |
| | | 1.57 | 0.93 | 1.08 | 1.17 |
| | TG | 0.61 | 0.58 | 0.56 | 0.48 |
| | | 0.62 | 0.76 | 0.51 | 0.52 |
| | | 0.99 | 1.44 | 1.42 | 1.88 |
| | | 1.50 | 1.09 | 1.38 | 1.66 |
| WV-3534 | ApoC3 | 0.83 | 0.09 | 0.10 | 0.10 |
| | | 1.14 | 0.17 | 0.12 | 0.09 |
| | | 1.17 | 0.22 | 0.51 | 0.13 |
| | | 1.15 | 0.17 | 0.36 | 0.21 |
| | TG | 0.91 | 0.13 | 0.17 | 0.13 |
| | | 1.34 | 0.24 | 0.18 | 0.14 |
| | | 0.82 | 0.25 | 0.61 | 0.16 |
| | | 0.93 | 0.18 | 0.44 | 0.23 |
| WV-2816 | ApoC3 | 1.10 | 0.35 | 0.21 | 0.31 |
| | | 1.09 | 0.53 | 0.35 | 0.52 |
| | | 0.92 | 0.65 | 0.33 | 0.27 |
| | | 0.79 | 0.96 | 0.67 | 0.61 |
| | TG | 1.04 | 0.44 | 0.26 | 0.35 |
| | | 1.01 | 0.54 | 0.37 | 0.47 |
| | | 0.63 | 1.15 | 0.50 | 0.40 |
| | | 0.70 | 1.43 | 0.96 | 0.69 |
| WV-4125 | ApoC3 | 0.47 | 0.32 | 0.27 | 0.32 |
| | | 0.86 | 0.23 | 0.17 | 0.36 |
| | | 1.22 | 0.99 | 0.82 | 0.96 |
| | | 1.33 | 0.88 | 0.98 | 0.94 |
| | TG | 0.54 | 0.36 | 0.40 | 0.36 |
| | | 1.05 | 0.24 | 0.23 | 0.31 |
| | | 1.01 | 1.51 | 1.31 | 1.30 |
| | | 0.88 | 0.78 | 1.03 | 0.92 |
| WV-4127 | ApoC3 | 1.06 | 1.00 | 0.51 | 0.75 |
| | | 0.46 | 0.26 | 0.18 | 0.41 |
| | | 1.42 | 0.94 | 0.82 | 1.07 |
| | | 1.23 | 1.11 | 1.25 | 0.72 |
| | TG | 1.29 | 1.74 | 0.61 | 0.85 |
| | | 0.59 | 0.20 | 0.23 | 0.46 |
| | | 1.37 | 1.03 | 1.47 | 1.06 |
| | | 1.37 | 1.34 | 1.31 | 1.04 |
| WV-4128 | ApoC3 | 0.84 | 0.40 | 0.35 | 0.63 |
| | | 0.62 | 0.38 | 0.38 | 0.32 |
| | | 1.54 | 1.25 | 1.04 | 1.47 |
| | | 1.03 | 1.45 | 1.39 | 1.46 |
| | TG | 0.90 | 0.43 | 0.45 | 0.59 |
| | | 0.50 | 0.32 | 0.30 | 0.25 |
| | | 1.69 | 1.69 | 1.60 | 1.51 |
| | | 0.90 | 1.30 | 1.35 | 1.37 |
| WV-4129 | ApoC3 | 0.92 | 0.73 | 0.35 | 0.30 |
| | | 1.02 | 0.36 | 0.28 | 0.52 |
| | | 1.23 | 1.01 | 0.56 | 0.37 |
| | | 1.60 | 1.04 | 0.25 | 0.16 |
| | TG | 0.82 | 0.82 | 0.40 | 0.30 |
| | | 1.07 | 0.41 | 0.29 | 0.55 |
| | | 1.51 | 1.38 | 1.15 | 0.48 |
| | | 1.74 | 1.56 | 0.51 | 0.25 |
| WV-4132 | ApoC3 | 0.75 | 0.33 | 0.32 | 0.27 |
| | | 0.90 | 0.46 | 0.37 | 0.41 |
| | | 1.33 | 1.04 | 0.80 | 0.85 |
| | | 1.05 | 0.79 | 0.44 | 0.53 |
| | TG | 0.89 | 0.33 | 0.38 | 0.28 |
| | | 1.01 | 0.65 | 0.43 | 0.51 |
| | | 1.36 | 1.29 | 1.14 | 1.14 |
| | | 1.39 | 0.94 | 0.70 | 0.53 |
| WV-4133 | ApoC3 | 0.88 | 0.44 | 0.41 | 0.66 |
| | | 0.42 | 0.22 | 0.16 | 0.32 |
| | | 1.16 | 0.63 | 0.41 | 0.25 |
| | | 0.98 | 1.04 | 0.61 | 0.50 |
| | TG | 1.03 | 0.52 | 0.53 | 0.66 |
| | | 0.46 | 0.22 | 0.25 | 0.40 |
| | | 1.30 | 0.93 | 0.61 | 0.23 |
| | | 0.90 | 1.38 | 0.90 | 0.59 |
| WV-4134 | ApoC3 | 0.48 | 0.27 | 0.24 | 0.31 |
| | | 0.43 | 0.22 | 0.18 | 0.27 |
| | | 1.10 | 0.97 | 0.67 | 0.82 |
| | | 1.82 | 1.12 | 0.57 | 0.69 |
| | TG | 0.54 | 0.29 | 0.23 | 0.26 |
| | | 0.48 | 0.24 | 0.23 | 0.30 |
| | | 1.46 | 1.57 | 1.17 | 1.19 |
| | | 1.30 | 0.90 | 0.70 | 0.22 |

| | | −2 | 5 | 8 | 14 |
|---|---|---|---|---|---|
| PBS | ApoC3 | 0.622 | 0.747 | 0.784 | 0.661 |
| | | 0.407 | 0.439 | 0.464 | 0.532 |
| | | 2.096 | 1.034 | 1.411 | 1.373 |
| | | 1.852 | 0.96 | 1.111 | 1.506 |
| | TG | 0.631758 | 0.60746 | 0.546714 | 0.473818 |
| | | 0.461669 | 0.473818 | 0.413073 | 0.461669 |
| | | 1.627992 | 1.34856 | 1.688738 | 1.433605 |
| | | 2.10181 | 0.971935 | 1.457903 | 1.299964 |
| WV-3534 | ApoC3 | 0.581 | 0.096 | 0.129 | 0.116 |
| | | 0.709 | 0.167 | 0.117 | 0.146 |
| | | 2.935 | 0.993 | 0.518 | 0.148 |
| | | 1.537 | 0.799 | 0.319 | 0.194 |
| | TG | 0.741101 | 0.085044 | 0.14579 | 0.085044 |
| | | 0.959786 | 0.218685 | 0.14579 | 0.109343 |
| | | 1.385008 | 1.117726 | 0.619609 | 0.109343 |
| | | 1.166322 | 0.984085 | 0.388774 | 0.182238 |
| WV-2816 | ApoC3 | 0.869 | 0.365 | 0.246 | 0.278 |
| | | 0.737 | 0.411 | 0.298 | 0.441 |
| | | 1.953 | 0.828 | 0.353 | 0.369 |
| | | 1.354 | 1.361 | 0.739 | 0.734 |
| | TG | 0.75325 | 0.400923 | 0.242984 | 0.255133 |
| | | 0.959786 | 0.437371 | 0.315879 | 0.388774 |
| | | 1.142024 | 1.069129 | 0.425222 | 0.291581 |
| | | 0.984085 | 1.336411 | 0.850443 | 0.571012 |
| WV-4126 | ApoC3 | 1.681 | 0.681 | 0.348 | 0.48 |
| | | 1.343 | 0.435 | 0.17 | 0.249 |
| | | 0.807 | 0.489 | 0.195 | 0.421 |
| | | 1.148 | 0.749 | 0.523 | 0.477 |
| | TG | 1.34856 | 0.862593 | 0.59531 | 0.388774 |
| | | 1.312113 | 0.425222 | 0.218685 | 0.182238 |
| | | 1.044831 | 0.510266 | 0.242984 | 0.400923 |
| | | 1.737334 | 0.59531 | 1.154173 | 0.388774 |
| WV-4130 | ApoC3 | 1.128 | 0.852 | 0.782 | 1.798 |
| | | 0.424 | 0.753 | 0.301 | 0.445 |
| | | 2.74 | 0.6 | 0.084 | 0.281 |
| | | 0.192 | 0.084 | 0.12 | 0.165 |
| | TG | 0.631758 | 0.328028 | 0.315879 | 0.242984 |
| | | 0.583161 | 0.218685 | 0.157939 | 0.085044 |
| | | 1.154173 | 0.279431 | 0.218685 | 0.352327 |
| | | 1.190621 | 0.291581 | 0.170089 | 0.194387 |
| WV-4131 | ApoC3 | 0.311 | 0.223 | 0.175 | 0.344 |
| | | 0.128 | 0.09 | 0.05 | 0.139 |
| | | 1.13 | 2.182 | 0.271 | 0.09 |
| | | 1.334 | 1.01 | 0.631 | 0.458 |
| | TG | 0.388774 | 0.157939 | 0.230835 | 0.218685 |
| | | 0.206536 | 0.14579 | 0.097194 | 0.109343 |
| | | 1.105576 | 1.008383 | 0.315879 | 0.230835 |
| | | 0.668206 | 0.376625 | 0.072895 | 0.109343 |
| WV-4135 | ApoC3 | 2.181 | 1.22 | 0.526 | 0.575 |
| | | 1.996 | 1.102 | 0.691 | 0.812 |
| | | 1.418 | 0.379 | 0.187 | 0.271 |
| | | 2.081 | 0.594 | 0.213 | 0.088 |
| | TG | 0.692504 | 0.255133 | 0.182238 | 0.267282 |
| | | 0.75325 | 0.352327 | 0.206536 | 0.255133 |
| | | 1.117726 | 0.60746 | 0.352327 | 0.255133 |
| | | 0.935488 | 0.704653 | 0.30373 | 0.255133 |
| WV-4136 | ApoC3 | 1.013 | 0.132 | 0.067 | 0.049 |
| | | 0.663 | 0.211 | 0.171 | 0.271 |
| | | 0.527 | 1.451 | 0.817 | 0.232 |
| | | 1.748 | 1.263 | 0.511 | 0.453 |
| | TG | 0.911189 | 0.267282 | 0.121492 | 0.109343 |
| | | 0.182238* | 0.072895* | 0.072895* | 0.060746* |
| | | 0.947637 | 0.546714 | 0.388774 | 0.109343 |
| | | 0.534564 | 0.364476 | 0.206536 | 0.085044 |

### Relative to PBS.

TABLE 60K
Activity of oligonucleotides

| | −2 | 4 | 7 | 14 | 21 | 28 | 39 |
|---|---|---|---|---|---|---|---|
| WV-2141 | 1.49 | 0.67 | 0.37 | 0.56 | 0.80 | 0.79 | 0.75 |
| | 0.64 | 0.32 | 0.17 | 0.32 | | 0.55 | 0.91 |
| | 1.21 | 0.76 | 0.60 | 0.87 | 1.11 | 1.13 | 1.24 |
| | 0.88 | 0.81 | 0.57 | 0.33 | 0.99 | 0.85 | 0.83 |
| WV-3968 | 0.80 | 0.13 | 0.11 | 0.16 | 0.33 | 0.36 | |
| | 0.77 | 0.09 | 0.08 | 0.11 | 0.27 | 0.37 | 0.73 |
| | 2.00 | 0.32 | 0.13 | 0.11 | | 0.26 | 0.79 |
| | 0.81 | 0.06 | 0.03 | 0.07 | 0.17 | 0.29 | 0.43 |
| WV-3534 | 1.11 | 0.15 | 0.11 | 0.13 | 0.43 | 0.58 | 0.74 |
| | 1.08 | 0.14 | 0.11 | 0.11 | 0.38 | 0.45 | 0.66 |
| | 1.61 | 0.41 | 0.21 | 0.18 | 0.43 | 0.67 | 1.14 |
| | 0.00 | 0.25 | 0.18 | 0.15 | 0.48 | 0.78 | 1.11 |
| | 1.00 | 0.65 | 0.08 | 0.09 | 0.32 | 0.52 | 1.01 |

Animals were dosed twice, subcutaneously, on days 1 and 4 at 10 mpk. Tested samples were withdrawn on days 2, 4, 7, 14, 21, 28 and 39.

TABLE 60L

| Conc. | 1 | 0.522 | 0.045 | −0.43 | −0.90 | −1.38 | −1.86 | −2.33 | −2.81 | −3.29 | −3.77 |
|---|---|---|---|---|---|---|---|---|---|---|---|
| WV-7540 | 0.133 | 0.158 | 0.269 | 0.395 | 0.559 | 0.688 | 0.726 | 0.693 | 0.720 | 0.774 | 0.637 |
| | 0.153 | 0.251 | 0.325 | 0.470 | 0.644 | 0.798 | 0.921 | 0.839 | 0.923 | 1.343 | 1.046 |
| WV-8427 | 0.202 | 0.225 | 0.372 | 0.400 | 0.634 | 0.802 | 0.913 | 1.000 | 1.004 | 0.910 | 0.880 |
| | 0.168 | 0.258 | 0.354 | 0.543 | 0.714 | 0.923 | 0.972 | 0.982 | 1.038 | 1.596 | 1.252 |
| WV-8429 | 0.140 | 0.150 | 0.255 | 0.276 | 0.455 | 0.609 | 0.788 | 1.011 | 0.948 | 0.968 | 1.307 |
| | 0.148 | 0.187 | 0.218 | 0.297 | 0.491 | 0.708 | 0.964 | 0.948 | 1.006 | 0.954 | 0.959 |
| WV-8431 | 0.167 | 0.205 | 0.234 | 0.288 | 0.494 | 0.755 | 0.896 | 1.000 | 1.055 | 1.117 | 0.953 |
| | 0.209 | 0.234 | 0.275 | 0.338 | 0.504 | 0.713 | 0.913 | 1.078 | 0.972 | 1.023 | 0.914 |
| WV-6439 | 0.206 | 0.172 | 0.217 | 0.232 | 0.521 | 0.740 | 0.909 | 0.993 | 1.052 | 0.948 | 0.952 |
| | 0.198 | 0.301 | 0.239 | 0.552 | 0.567 | 0.747 | 1.010 | 1.254 | 1.038 | 1.334 | 1.212 |
| WV-6439 | 0.190 | 0.176 | 0.189 | 0.265 | 0.400 | 0.566 | 0.703 | 0.797 | 0.940 | 0.985 | 0.864 |
| | 0.209 | 0.217 | 0.226 | 0.226 | 0.398 | 0.635 | 0.915 | 1.040 | 1.025 | 1.142 | 1.019 |
| WV-6431 | 0.209 | 0.283 | 0.185 | 0.227 | 0.415 | 0.668 | 0.728 | 0.850 | 1.053 | 0.899 | 0.927 |
| | 0.220 | 0.191 | 0.202 | 0.206 | 0.388 | 0.622 | 0.807 | 0.944 | 0.891 | 0.950 | 1.032 |
| WV-6439 | 0.198 | 0.208 | 0.229 | 0.285 | 0.542 | 0.670 | 0.749 | 0.852 | 0.775 | 0.855 | 0.863 |
| Plate Control | 0.206 | 0.183 | 0.232 | 0.454 | 0.520 | 0.736 | 0.935 | 0.986 | 0.998 | 1.099 | 0.890 |

Concentrations (Conc.) are provided in nM, exp 10.

TABLE 60M
Activity of oligonucleotides

| | PBS | WV-6544 | WV-6558 | WV-6559 |
|---|---|---|---|---|
| 1 mpk | 1.269 | 1.314 | 0.252 | 0.387 |
| | 1.144 | 0.529 | 0.085 | 0.302 |
| | 0.865 | 0.554 | 0.077 | 0.219 |
| | 0.914 | 0.374 | 0.108 | 0.299 |
| | 0.808 | 0.416 | 0.206 | 0.402 |
| 3 mpk | | 0.199 | 0.106 | 0.194 |
| | | 0.25 | 0.189 | 0.257 |
| | | 0.182 | 0.091 | 0.388 |
| | | 0.102 | 0.064 | 0.232 |
| | | 0.146 | 0.044 | 0.065 |
| 10 mpk | | 0.066 | 0.058 | 0.088 |
| | | 0.091 | 0.058 | 0.065 |
| | | 0.067 | 0.061 | 0.067 |
| | | 0.151 | 0.094 | 0.075 |
| | | 0.099 | 0.103 | 0.09 |

Single IV dosage. Numbers are APOC3 mRNA level at 15 days. APOC3 levels were also reduced at 8 days (data not shown).

TABLE 60N

| | 1 | 8 | 15 | 22 | 29 | 36 | 43 | 50 | 57 | 63 |
|---|---|---|---|---|---|---|---|---|---|---|
| PBS | 0.53 | 0.27 | 0.88 | 0.89 | 1.55 | 1.66 | 0.68 | 1.21 | 0.87 | 1.29 |
| | 1.23 | 1.49 | 1.42 | 1.19 | 1.13 | 1.19 | 1.12 | 0.96 | 1.31 | 1.09 |
| | 0.65 | 0.83 | 1.06 | 0.86 | 0.88 | 0.59 | 0.67 | 1.46 | 1.19 | 0.73 |
| | 1.26 | 0.98 | 1.15 | 1.94 | 1.21 | 1.23 | 1.99 | 0.82 | 1.26 | 1.36 |
| | 1.34 | 1.42 | 0.50 | 0.12 | 0.24 | 0.33 | 0.54 | 0.54 | 0.38 | 0.53 |
| WV-3968 | 2.60 | 0.13 | 0.04 | 0.06 | 0.43 | 0.47 | 1.76 | 1.29 | 1.51 | 1.37 |
| | 2.18 | 0.12 | 0.10 | 0.52 | 0.68 | 1.46 | 1.88 | 0.71 | 1.51 | 1.78 |
| | 1.67 | 0.01 | 0.05 | 0.07 | 0.29 | 0.57 | 1.83 | 0.91 | 0.68 | 1.43 |
| | 1.14 | 0.01 | 0.03 | 0.03 | 0.26 | 0.56 | 1.05 | 1.23 | 0.88 | 0.70 |
| | 1.11 | 0.02 | 0.02 | 0.03 | 0.19 | 0.55 | 0.81 | 1.27 | 1.56 | 1.30 |
| WV-6003 | 1.60 | 0.04 | 0.06 | 0.12 | 0.12 | 0.31 | 0.45 | 0.54 | 0.94 | 1.52 |
| | 0.40 | 0.04 | 0.06 | 0.08 | 0.14 | 0.16 | 0.45 | 0.23 | 0.68 | 1.04 |
| | 1.30 | 0.03 | 0.05 | 0.08 | 0.25 | 0.12 | 0.33 | 0.24 | 0.44 | 0.58 |
| | 1.33 | 0.04 | 0.05 | 0.13 | 0.34 | 0.09 | 0.43 | 0.34 | 0.73 | 1.56 |
| | 1.11 | 0.04 | 0.06 | 0.00 | 0.15 | 0.09 | 0.31 | 0.19 | 0.70 | 0.87 |
| WV-6555 | 0.95 | 0.07 | 0.05 | 0.24 | 0.57 | 0.37 | 1.05 | 0.46 | 0.54 | 1.07 |
| | 0.14 | 0.03 | 0.04 | 0.04 | 0.17 | 0.09 | 0.35 | 0.39 | 0.27 | 0.42 |
| | 1.70 | 0.09 | 0.06 | 0.11 | 0.25 | 0.87 | 0.84 | 1.69 | 1.07 | 2.46 |
| | 0.29 | 0.02 | 0.04 | 0.08 | 0.27 | 0.34 | 0.61 | 0.47 | 0.29 | 0.97 |
| | 0.75 | 0.05 | 0.09 | 0.19 | 0.67 | 0.77 | 0.94 | 0.79 | 0.85 | 1.18 |
| WV-6757 | 1.08 | 0.04 | 0.06 | 0.10 | 0.43 | 0.24 | 0.39 | 0.27 | 0.22 | 0.42 |
| | 0.72 | 0.04 | 0.04 | 0.07 | 0.18 | 0.13 | 0.44 | 0.26 | 0.29 | 0.35 |
| | 0.79 | 0.02 | 0.00 | 0.37 | 0.12 | 0.13 | 0.32 | 0.34 | 0.37 | 0.81 |
| | 2.78 | 0.25 | 0.20 | 0.36 | 0.49 | 1.05 | 1.13 | 0.87 | 0.48 | 1.67 |
| | 0.81 | 0.08 | 0.06 | 0.07 | 0.44 | 0.42 | 0.96 | 0.53 | 0.68 | 1.01 |
| WV-6544 | 0.88 | 0.04 | 0.03 | 0.02 | 0.05 | 0.51 | 0.96 | 1.28 | 1.88 | 1.90 |
| | 0.55 | 0.02 | 0.05 | 0.04 | 0.17 | 0.32 | 1.50 | 1.15 | 1.19 | 2.47 |
| | 0.71 | 0.02 | 0.03 | 0.04 | 0.08 | 0.82 | 0.82 | 1.46 | 1.54 | 1.49 |
| | 0.78 | 0.03 | 0.00 | 0.01 | 0.14 | 0.18 | 0.60 | 0.33 | 0.24 | 0.49 |
| | 1.38 | 0.13 | 0.03 | 0.02 | 0.35 | 0.37 | 1.05 | 0.75 | 0.92 | 1.64 |
| WV-6558 | 0.94 | 0.01 | 0.01 | 0.01 | 0.02 | 0.05 | 0.22 | 0.06 | 0.16 | 0.56 |
| | 1.00 | 0.01 | 0.05 | 0.04 | 0.27 | 0.19 | 0.58 | 0.69 | 0.75 | 1.29 |
| | 0.59 | 0.03 | 0.03 | 0.02 | 0.04 | 0.04 | 0.23 | 0.11 | 0.13 | 0.16 |
| | 1.39 | 0.02 | 0.03 | 0.02 | 0.04 | 0.14 | 0.27 | 0.25 | 0.46 | 1.15 |
| | 0.93 | 0.05 | 0.05 | 1.50 | 0.05 | 0.05 | 0.28 | 0.29 | 0.49 | 1.22 |
| WV-6559 | 0.70 | 0.02 | 0.04 | 0.06 | 0.05 | 0.08 | 0.25 | 0.28 | 0.51 | 1.33 |
| | 1.64 | 0.06 | 0.01 | 0.05 | 0.08 | 0.05 | 0.22 | 0.29 | 0.12 | 0.50 |
| | 1.06 | 0.01 | 0.00 | 0.04 | 0.03 | 0.04 | 0.24 | 0.00 | 0.02 | 0.01 |
| | 1.53 | 0.21 | 0.02 | 0.05 | 0.13 | 0.17 | 0.32 | 0.71 | 0.69 | 1.92 |
| | 0.60 | 0.01 | 0.03 | 0.02 | 0.05 | 0.07 | 0.30 | 0.36 | 0.18 | 0.40 |

hAPOC3 Tg (transgenic) mice were dosed with 5 mpk of oligonucleotide on days 1 and 3, and samples were collected on days 1, 8, 15, 22, 29, 43, 50, 57 and 63. Levels of hAPOC3 protein level relative to PBS are shown.

TABLE 60O

| | 1 | 8 | 15 | 22 | 29 | 36 | 43 | 50 | 57 | 63 |
|---|---|---|---|---|---|---|---|---|---|---|
| PBS | 0.53 | 0.27 | 0.88 | 0.89 | 1.55 | 1.66 | 0.68 | 1.21 | 0.87 | 1.29 |
| | 1.23 | 1.49 | 1.42 | 1.19 | 1.13 | 1.19 | 1.12 | 0.96 | 1.31 | 1.09 |
| | 0.65 | 0.83 | 1.06 | 0.86 | 0.88 | 0.59 | 0.67 | 1.46 | 1.19 | 0.73 |
| | 1.26 | 0.98 | 1.15 | 1.94 | 1.21 | 1.23 | 1.99 | 0.82 | 1.26 | 1.36 |
| | 1.34 | 1.42 | 0.50 | 0.12 | 0.24 | 0.33 | 0.54 | 0.54 | 0.38 | 0.53 |
| WV-3968 | 2.60 | 0.13 | 0.04 | 0.06 | 0.43 | 0.47 | 1.76 | 1.29 | 1.51 | 1.37 |
| | 2.18 | 0.12 | 0.10 | 0.52 | 0.68 | 1.46 | 1.88 | 0.71 | 1.51 | 1.78 |
| | 1.67 | 0.01 | 0.05 | 0.07 | 0.29 | 0.57 | 1.83 | 0.91 | 0.68 | 1.43 |
| | 1.14 | 0.01 | 0.03 | 0.03 | 0.26 | 0.56 | 1.05 | 1.23 | 0.88 | 0.70 |
| | 1.11 | 0.02 | 0.02 | 0.03 | 0.19 | 0.55 | 0.81 | 1.27 | 1.56 | 1.30 |
| WV-6003 | 1.60 | 0.04 | 0.06 | 0.12 | 0.12 | 0.31 | 0.45 | 0.54 | 0.94 | 1.52 |
| | 0.40 | 0.04 | 0.06 | 0.08 | 0.14 | 0.16 | 0.45 | 0.23 | 0.68 | 1.04 |
| | 1.30 | 0.03 | 0.05 | 0.08 | 0.25 | 0.12 | 0.33 | 0.24 | 0.44 | 0.58 |
| | 1.33 | 0.04 | 0.05 | 0.13 | 0.34 | 0.09 | 0.43 | 0.34 | 0.73 | 1.56 |
| | 1.11 | 0.04 | 0.06 | 0.00 | 0.15 | 0.09 | 0.31 | 0.19 | 0.70 | 0.87 |
| WV-6555 | 0.95 | 0.07 | 0.05 | 0.24 | 0.57 | 0.37 | 1.05 | 0.46 | 0.54 | 1.07 |
| | 0.14 | 0.03 | 0.04 | 0.04 | 0.17 | 0.09 | 0.35 | 0.39 | 0.27 | 0.42 |
| | 1.70 | 0.09 | 0.06 | 0.11 | 0.25 | 0.87 | 0.84 | 1.69 | 1.07 | 2.46 |
| | 0.29 | 0.02 | 0.04 | 0.08 | 0.27 | 0.34 | 0.61 | 0.47 | 0.29 | 0.97 |
| | 0.75 | 0.05 | 0.09 | 0.19 | 0.67 | 0.77 | 0.94 | 0.79 | 0.85 | 1.18 |
| WV-6757 | 1.08 | 0.04 | 0.06 | 0.10 | 0.43 | 0.24 | 0.39 | 0.27 | 0.22 | 0.42 |
| | 0.72 | 0.04 | 0.04 | 0.07 | 0.18 | 0.13 | 0.44 | 0.26 | 0.29 | 0.35 |
| | 0.79 | 0.02 | 0.00 | 0.37 | 0.12 | 0.13 | 0.32 | 0.34 | 0.37 | 0.81 |
| | 2.78 | 0.25 | 0.20 | 0.36 | 0.49 | 1.05 | 1.13 | 0.87 | 0.48 | 1.67 |
| | 0.81 | 0.08 | 0.06 | 0.07 | 0.44 | 0.42 | 0.96 | 0.53 | 0.68 | 1.01 |
| WV-6544 | 0.88 | 0.04 | 0.03 | 0.02 | 0.05 | 0.51 | 0.96 | 1.28 | 1.88 | 1.90 |
| | 0.55 | 0.02 | 0.05 | 0.04 | 0.17 | 0.32 | 1.50 | 1.15 | 1.19 | 2.47 |
| | 0.71 | 0.02 | 0.03 | 0.04 | 0.08 | 0.82 | 0.82 | 1.46 | 1.54 | 1.49 |
| | 0.78 | 0.03 | 0.00 | 0.01 | 0.14 | 0.18 | 0.60 | 0.33 | 0.24 | 0.49 |
| | 1.38 | 0.13 | 0.03 | 0.02 | 0.35 | 0.37 | 1.05 | 0.75 | 0.92 | 1.64 |
| WV-6558 | 0.94 | 0.01 | 0.01 | 0.01 | 0.02 | 0.05 | 0.22 | 0.06 | 0.16 | 0.56 |
| | 1.00 | 0.01 | 0.05 | 0.04 | 0.27 | 0.19 | 0.58 | 0.69 | 0.75 | 1.29 |
| | 0.59 | 0.03 | 0.03 | 0.02 | 0.04 | 0.04 | 0.23 | 0.11 | 0.13 | 0.16 |
| | 1.39 | 0.02 | 0.03 | 0.02 | 0.04 | 0.14 | 0.27 | 0.25 | 0.46 | 1.15 |
| | 0.93 | 0.05 | 0.05 | 1.50 | 0.05 | 0.05 | 0.28 | 0.29 | 0.49 | 1.22 |
| WV-6559 | 0.70 | 0.02 | 0.04 | 0.06 | 0.05 | 0.08 | 0.25 | 0.28 | 0.51 | 1.33 |
| | 1.64 | 0.06 | 0.01 | 0.05 | 0.08 | 0.05 | 0.22 | 0.29 | 0.12 | 0.50 |
| | 1.06 | 0.01 | 0.00 | 0.04 | 0.03 | 0.04 | 0.24 | 0.00 | 0.02 | 0.01 |
| | 1.53 | 0.21 | 0.02 | 0.05 | 0.13 | 0.17 | 0.32 | 0.71 | 0.69 | 1.92 |
| | 0.60 | 0.01 | 0.03 | 0.02 | 0.05 | 0.07 | 0.30 | 0.36 | 0.18 | 0.40 |

hAPOC3 Tg (transgenic) mice were dosed with 5 mpk of oligonucleotide on days 1 and 3, and samples were collected on days 1, 8, 15, 22, 29, 43, 50, 57 and 63. Levels of hAPOC3 protein level relative to PBS are shown.

TABLE 60P
Part I. Oligonucleotides

| | | | | SEQ |
|---|---|---|---|---|
| Oligo- | | | Stereo- | ID |
| nucleotide | Sequence | Naked Sequence | chemistry | NO: |
| WV-7297 | Mod038L001Teo * Geo * Geo * Teo * Aeo | TGGTAA TCCACTT | OXXXXXXXXXX | 1953 |
| | * A * T * m5C * m5C * A * m5C * T * T * T | TCAGAGG | XXXXXXXXX | |
| | * m5C * Aeo * Geo * Aeo * Geo * Geo | | | |
| WV-7298 | Mod039L001Teo * Geo * Geo * Teo * Aeo | TGGTAA TCCACTT | OXXXXXXXXXX | 1954 |
| | * A * T * m5C * m5C * A * m5C * T * T * T | TCAGAGG | XXXXXXXXX | |
| | * m5C * Aeo * Geo * Aeo * Geo * Geo | | | |
| WV-7299 | Mod040L001Teo * Geo * Geo * Teo * Aeo | TGGTAA TCCACTT | OXXXXXXXXXX | 1955 |
| | * A * T * m5C * m5C * A * m5C * T * T * T | TCAGAGG | XXXXXXXXX | |
| | * m5C * Aeo * Geo * Aeo * Geo * Geo | | | |
| WV-7300 | Mod041L001Teo * Geo * Geo * Teo * Aeo | TGGTAA TCCACTT | OXXXXXXXXXX | 1956 |
| | * A * T * m5C * m5C * A * m5C * T * T * T | TCAGAGG | XXXXXXXXX | |
| | * m5C * Aeo * Geo * Aeo * Geo * Geo | | | |
| WV-5287 | Mod034L001Teo * Geo * Geo * Teo * Aeo | TGGTAA TCCACTT | OXXXXXXXXXX | 1957 |
| | * A * T * m5C * m5C * A * m5C * T * T * T | TCAGAGG | XXXXXXXXX | |
| | * m5C * Aeo * Geo * Aeo * Geo * Geo | | | |

Several oligonucleotides were also prepared which target a mouse homolog of different gene, Factor XI (FXI), and which comprised an additional component, which was a tri-, bi- or mono-antennary ligand which was either a GalNAc or a PFE ligand.
The various components (e.g., *, Mod038, etc.) in this table are the same as those in Table 1A. All of these oligonucleotides are single-stranded, though the sequences are split into multiple lines for formatting.

TABLE 60P
Part II. Activity of oligonucleotides.

| mFXI Oligonucleotide | Ligand | mFX1/mHPRT1 |
|---|---|---|
| WV-3969 | Tri-GalNAc | 23 |
| WV-5287 | Tri-PFE ligand | 22 |
| WV-7299 | Bis-GalNAc | 22 |
| WV-7300 | Bis-PFE ligand | 20 |
| WV-7297 | Mono-GalNAc | 74 |
| WV-7298 | Mono-PFE ligand | 43 |

The oligonucleotides listed in Table 60P, Part I, were administered to mice at 0.3, 1 or 3 mpK QDx3. Numbers below represent the mFXI/mHPRT1 mRNA level relative to control at 3 mpk. Mice were also administered oligonucleotides at 0.3 and 1 mpk (data not shown).

While not wishing to be bound by any particular theory, the present disclosure notes that further experiments also provided additional data supporting the conclusions that various putative single-stranded RNAi agents were, in fact, capable of mediating RNA interference; and that various oligonucleotides designed to be capable of mediating knockdown via a RNaseH-mediated mechanism in fact mediated knockdown via a RNaseH-mediated mechanism.

In one experiment, an in vitro RNase H assay was performed, with APOC3 oligonucleotide WV-1868 (ASO, mediating a RNase H knockdown mechanism of APOC3) as a positive control, and APOC3 oligonucleotide WV-2110 (a single-stranded RNAi agent) as a negative control. RNA molecule WV-2372 is used as a test substrate. In the RNase H assay, dual mechanism APOC3 oligonucleotide WV-2111 mediated RNase H knockdown (data not shown).

In another experiment, an in vitro Ago-2 assay (for single-stranded RNA interference) was performed. This assay was performed with single-stranded RNAi agents to APOC3.

A RNA test substrate was WV-2372 (APOC3). In the results, the band representing the RNA test substrate is absent in the presence of APOC3 oligonucleotides WV-1308 and WV-2420, indicating that these oligonucleotides are single-stranded RNAi agents capable of mediating RNA interference. Various controls were used: Substrate in the absence of negative control ASO WV-2134; substrate in the presence of negative control ASO WV-2134, which does not mediate RNA interference; substrate in the absence of test oligonucleotide WV-1308; substrate in the absence of test oligonucleotide WV-2420; substrate alone; no substrate, with added WV-2134; and no substrate, with added WV-1308 (data not shown).

In another experiment, in vitro Ago-2 assay was (for single-stranded RNA interference) performed, using an APOC3 mRNA as a test substrate in a 3′ RACE assay in Hep3B cells. A cleavage product of the APOC3 mRNA in the presence of test oligonucleotide WV-3021 was detected, corresponding to cleavage of the mRNA at a site corresponding to a cut between positions 10 and 11 of WV-3021 (data not shown), which result is consistent with RNA interference. An artifactual cleavage product was also detected.

In other experiments, dual mechanism (hybrid format) APOC3 oligonucleotide WV-2111 was shown to be capable of mediating knockdown by both RNase H and RNA interference. A RNA substrate for WV-2111, which comprises the sequence of GCUGGCCUCCCAAUAAAGCUGGACA (SEQ ID NO: 1958), which is complementary to the sequence of APOC3 oligonucleotide WV-2111, was found to be cleaved in the presence of WV-2111 at the following positions: GC/UGGC/C/U/CC/CAAUA//AAGCUGGACA (SEQ ID NO: 1959), wherein / indicates a cleavage site in a position typical of RNaseH activity, and // indicates a cleavage site in a position typical of Ago-2 (RNA interference) activity. These data support the idea that WV-2111 mediates knockdown via both RNaseH and RNA interference mechanisms.

Several oligonucleotides were also found to be capable of mediating RNA interference in an Ago-2 in vitro assay. A RNA test substrate was APOC3 oligonucleotide WV-2372; this substrate disappeared in the presence of APOC3 oligonucleotides WV-1308, WV-2114, WV-2386, or WV-2387 (each tested separately), indicating that each of these oligonucleotides is capable of acting as single-stranded RNAi agents mediating RNA interference.

While not wishing to be bound by any particular theory, the present disclosure suggests that at least some of the oligonucleotides designated herein as single-stranded RNAi agents mediate knockdown via a RISC (RNA interference silencing complex); however, in at least some experiments, oligonucleotides designated herein as single-stranded RNAi agents were capable of mediating an observed knockdown of the protein level of a target greater than the observed knockdown of the corresponding mRNA level, and, while not wishing to be bound by any particular theory, the present disclosure suggests that this observation is consistent with the conjecture that some oligonucleotides designated herein as single-stranded RNAi agents which are capable of knocking down of a target gene or protein may be able to do so via a RISC-mediated mechanism and/or steric hindrance.

The present disclosure presents many non-limiting examples of oligonucleotides, having any of various sequences, formats, modifications, 5′-end regions, seed regions, post-seed regions, and 3′-end regions, and which are capable of mediating single-stranded RNA interference (e.g., single-stranded RNAi agents).

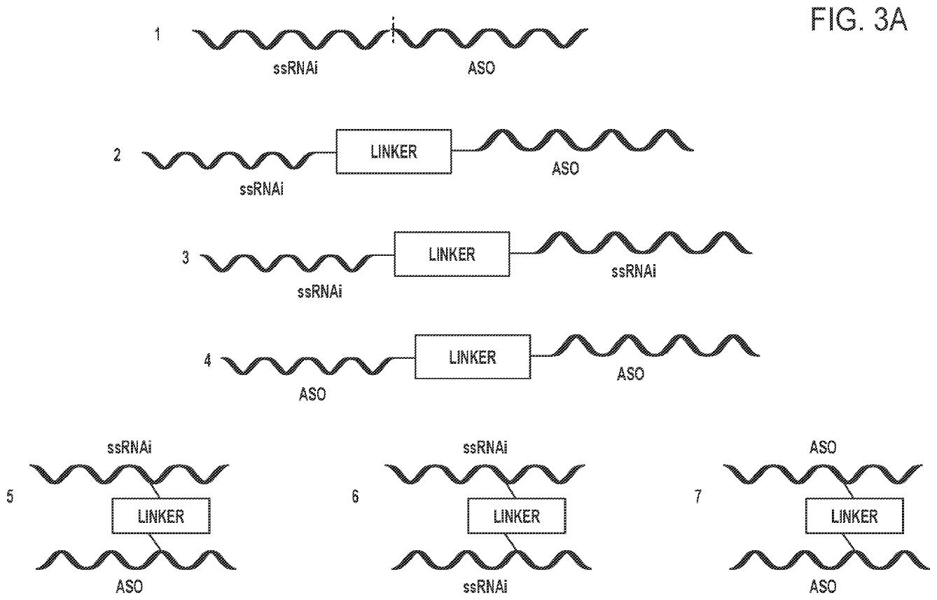

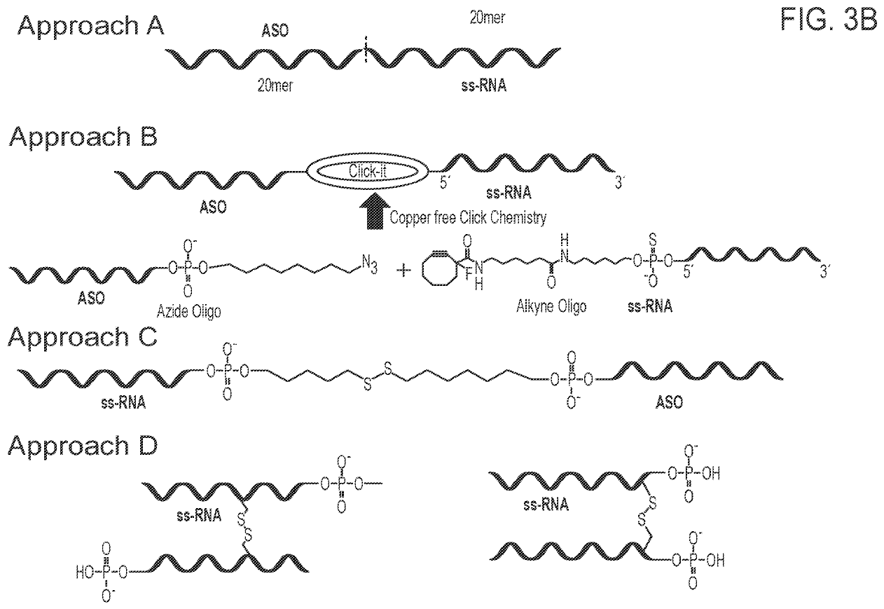

FIG. 3. FIGS. 3A and 3B show example multimer formats. Oligonucleotides can be joined directly and/or through linkers. As illustrated, a multimer can comprise oligonucleotide monomers of the same or different structures/types. In some embodiments, a monomer of a multimer is an ssRNAi agent. In some embodiments, a monomer of a multimer is a RNase H-dependent antisense oligonucleotide (ASO). Monomers can be joined through various positions, for example, the 5′-end, the 3′-end, or positions in between.

Shown directly below is a phosphoramidite useful for linking oligonucleotide monomers through formation of disulfide linkers. After incorporation into oligonucleotide monomers, a thioester can be hydrolyzed to release a free thiol, which can react with a thiol of another oligonucleotide monomer to form a disulfide bond, thereby linking oligonucleotide monomers together. Multiple thiol groups may be incorporated into oligonucleotides so that multimers of various numbers of monomers may be formed.

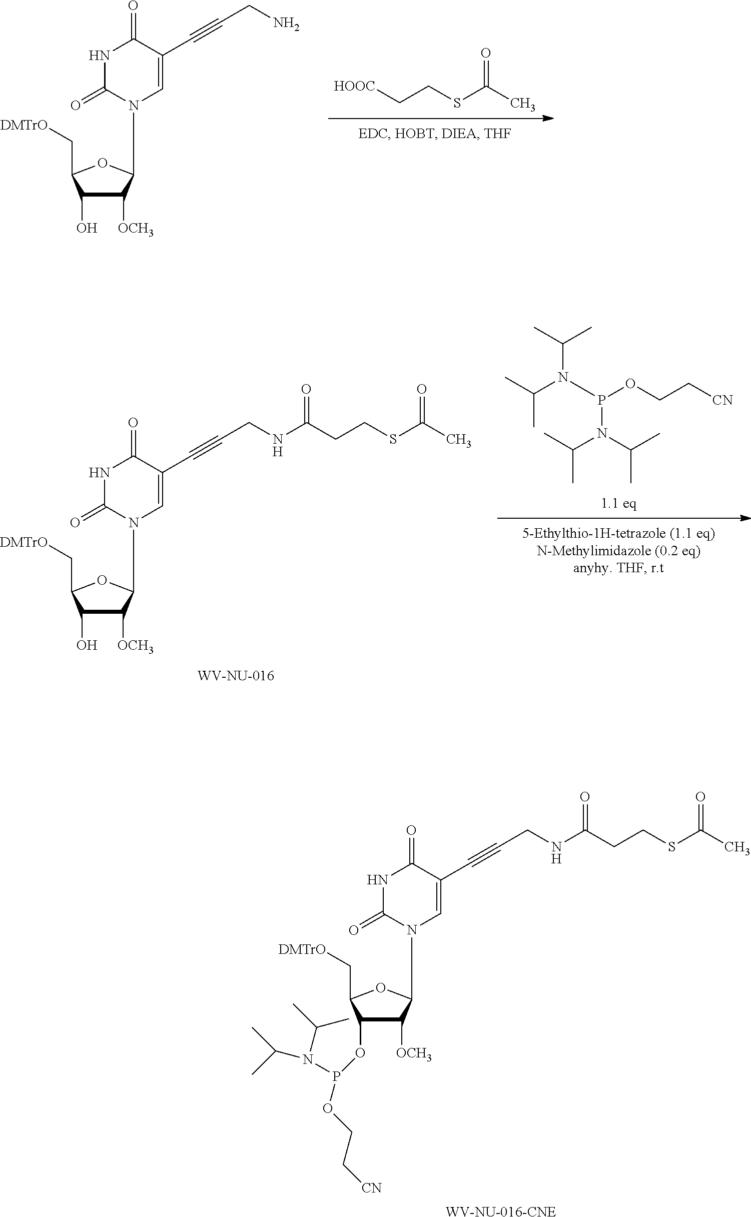

Table 90 shows in vitro allele-specific suppression of different oligonucleotides which target PNPLA3. Example oligonucleotides are completely complementary to target sequences of one allele, which target sequences comprise one or two SNP sites. One SNP site is associated with I148M change in protein sequence. Oligonucleotides comprising target-binding sequences that are completely complementary to target sequences comprising both SNPs were assessed in Hep3B cells (wild-type, C and C, I148) and Huh7 cells (with double mutation, T and G, M148). The double mutation was tested at various positions (8 and 11; 9 and 12; 10 and 13; etc.) and with various modifications to identify oligonucleotides capable of allele-specific knockdown of PNPLA3.

As shown in Table 90B, WV-7778 to WV-7793 and WV-3858 to WV-3864 were tested. In these oligonucleotides, the first and the last internucleotidic linkages in the wings are stereorandom PS and the others are PO; the 5′ wing and the 3′ wing comprise 2′-OMe. Several oligonucleotides demonstrated allele-specific knockdown of PNPLA3.

TABLE 90B
Activity of oligonucleotides.

| | |
|---|---|
| Hep3B | Huh7 |

| mock | 100 | 100 | 100 | 100 |
|---|---|---|---|---|
| WV-7778 | 92.8 | 86.9 | 63.6 | 52.7 |
| WV-7779 | 92.6 | 85.8 | 59.2 | 48.0 |
| WV-7780 | 102.3 | 97.1 | 36.4 | 34.8 |
| WV-7781 | 111.8 | 102.4 | 46.1 | 38.9 |
| WV-7782 | 108.5 | 94.7 | 43.3 | 35.0 |
| WV-7783 | 110.2 | 97.5 | 36.6 | 33.7 |
| WV-7784 | 105.0 | 95.9 | 41.2 | 41.9 |
| WV-7785 | 108.7 | 107.4 | 73.8 | 57.5 |
| WV-3858 | 103.3 | 103.6 | 55.1 | 52.3 |
| WV-3859 | 94.7 | 96.1 | 41.8 | 41.2 |
| WV-3860 | 104.2 | 94.1 | 46.7 | 45.6 |
| WV-3861 | 101.8 | 99.2 | 47.1 | 45.2 |
| WV-3862 | 97.1 | 96.5 | 44.0 | 42.3 |
| WV-3863 | 99.7 | 86.0 | 42.0 | 51.5 |
| WV-3864 | 97.8 | 96.8 | 53.5 | 38.9 |
| WV-7786 | 99.1 | 104.3 | 37.0 | 38.0 |
| WV-7787 | 84.2 | 87.5 | 23.0 | 25.1 |
| WV-7788 | 80.4 | 88.0 | 35.5 | 28.5 |
| WV-7789 | 82.7 | 85.7 | 25.4 | 25.5 |
| WV-7790 | 80.8 | 85.1 | 27.4 | 29.8 |
| WV-7791 | 87.1 | 80.1 | 40.8 | 42.1 |
| WV-7792 | 78.1 | 73.1 | 42.5 | 46.5 |
| WV-7793 | 68.3 | 65.1 | 49.9 | 44.9 |

As shown in Table 90C, WV-7794 to WV-7816 were tested. In these oligonucleotides, the first and the last internucleotidic linkages in the wings are stereorandom PS and the others are PO; the 5′ wing and the 3′ wing comprise 2′-MOE. Several oligonucleotides demonstrated allele-specific knockdown of PNPLA3.

TABLE 90C
Activity of oligonucleotides.

| | |
|---|---|
| Hep3B | Huh7 |

| mock | 100 | 100 | 100 | 100 |
|---|---|---|---|---|
| WV-7794 | 47.6 | 44.5 | 25.0 | 20.6 |
| WV-7795 | 58.5 | 52.8 | 14.3 | 14.8 |
| WV-7796 | 54.6 | 56.4 | 16.3 | 15.5 |
| WV-7797 | 75.1 | 74.2 | 13.5 | 12.8 |
| WV-7798 | 78.4 | 79.8 | 11.9 | 13.7 |
| WV-7799 | 89.9 | 92.4 | 23.5 | 25.7 |
| WV-7800 | 93.6 | 92.2 | 34.1 | 29.9 |
| WV-7801 | 90.3 | 90.3 | 38.4 | 29.3 |
| WV-7802 | 101.1 | 101.3 | 25.1 | 29.6 |
| WV-7803 | 102.0 | 103.2 | 24.8 | 25.8 |
| WV-7804 | 95.9 | 97.2 | 27.8 | 32.7 |
| WV-7805 | 100.5 | 95.5 | 21.9 | 22.0 |
| WV-7806 | 110.6 | 105.4 | 22.0 | 21.2 |
| WV-7807 | 96.2 | 101.5 | 21.1 | 23.8 |
| WV-7808 | 95.5 | 101.0 | 21.5 | 18.8 |
| WV-7809 | 85.3 | 84.2 | 17.1 | 15.7 |
| WV-7810 | 92.0 | 95.9 | 25.2 | 21.6 |
| WV-7811 | 100.1 | 100.0 | 26.6 | 27.1 |
| WV-7812 | 79.5 | 82.1 | 22.3 | 21.1 |
| WV-7813 | 83.7 | 76.2 | 23.7 | 18.2 |
| WV-7814 | 87.8 | 82.8 | 44.3 | 39.2 |
| WV-7815 | 78.1 | 74.8 | 45.3 | 37.1 |
| WV-7816 | 59.5 | 52.4 | 24.5 | 20.5 |

As shown in Table 90D, WV-7817 to WV-7839 were tested. In these oligonucleotides, the first and the last nucleotide are LNA; the 5′ wing has a LNA at the 5′ end of the oligonucleotide followed by several 2′-OMe; and the 3′ wing has several 2′-OMe followed by a LNA at the 3′ end of the oligonucleotide. Several oligonucleotides demonstrated allele-specific knockdown of PNPLA3.

TABLE 90D
Activity of oligonucleotides.

| | |
|---|---|
| Hep3B | Huh7 |

| mock | 100 | 100 | 100 | 100 |
|---|---|---|---|---|
| WV-7817 | 71.4 | 60.1 | 34.0 | 30.2 |
| WV-7818 | 68.1 | 75.8 | 44.0 | 30.0 |
| WV-7819 | 68.5 | 76.6 | 20.7 | 20.7 |
| WV-7820 | 87.7 | 86.0 | 24.0 | 22.4 |
| WV-7821 | 90.1 | 89.3 | 17.7 | 15.3 |
| WV-7822 | 101.2 | 87.2 | 19.6 | 11.7 |
| WV-7823 | 83.3 | 87.7 | 22.9 | 17.8 |
| WV-7824 | 99.0 | 101.9 | 31.1 | 31.6 |
| WV-7825 | 94.0 | 89.5 | 28.7 | 22.6 |
| WV-7826 | 95.6 | 87.5 | 21.2 | 17.6 |
| WV-7827 | 113.3 | 104.4 | 22.1 | 19.3 |
| WV-7828 | 108.1 | 102.8 | 25.7 | 23.6 |
| WV-7829 | 99.8 | 97.9 | 20.5 | 21.7 |
| WV-7830 | 95.9 | 87.8 | 18.5 | 19.2 |
| WV-7831 | 89.8 | 89.2 | 21.3 | 23.4 |
| WV-7832 | 76.2 | 71.7 | 9.4 | 11.8 |
| WV-7833 | 68.2 | 76.8 | 14.1 | 10.4 |
| WV-7834 | 69.5 | 71.2 | 17.4 | 16.5 |
| WV-7835 | 69.6 | 68.7 | 11.0 | 9.4 |
| WV-7836 | 59.8 | 67.8 | 18.3 | 21.0 |
| WV-7837 | 60.8 | 63.7 | 25.6 | 28.4 |
| WV-7838 | 48.2 | 50.5 | 16.8 | 13.5 |
| WV-7839 | 35.0 | 39.1 | 10.5 | 11.8 |

As shown in Table 90E, WV-7840 to WV-7862 were tested. In these oligonucleotides, the first and the last nucleotide are LNA; the 5′ wing has a LNA at the 5′ end of the oligonucleotide followed by several 2′-MOE; and the 3′ wing has several 2′-MOE (or 5-methyl 2′-MOE) followed by a LNA at the 3′ end of the oligonucleotide. Several oligonucleotides demonstrated allele-specific knockdown of PNPLA3.

TABLE 90E
Activity of oligonucleotides.

| | |
|---|---|
| Hep3B | Huh7 |

| mock | 100 | 100 | 100 | 100 |
|---|---|---|---|---|
| WV-7840 | 32.7 | 37.7 | 12.8 | 14.8 |
| WV-7841 | 45.9 | 49.8 | 8.1 | 10.6 |
| WV-7842 | 43.2 | 50.4 | 7.6 | 7.0 |
| WV-7843 | 53.7 | 61.0 | 8.7 | 10.6 |
| WV-7844 | 69.2 | 69.0 | 14.3 | 14.9 |
| WV-7845 | 80.8 | 83.7 | 15.0 | 13.7 |
| WV-7846 | 77.1 | 86.3 | 16.5 | 15.7 |
| WV-7847 | 85.2 | 96.4 | 18.5 | 15.6 |
| WV-7848 | 87.2 | 89.4 | 21.7 | 18.0 |
| WV-7849 | 65.0 | 74.2 | 17.2 | 16.7 |
| WV-7850 | 98.8 | 107.4 | 15.3 | 18.7 |
| WV-7851 | 105.0 | 95.8 | 11.4 | 15.9 |
| WV-7852 | 113.7 | 86.9 | 14.2 | 14.2 |
| WV-7853 | 108.5 | 90.7 | 10.1 | 14.3 |
| WV-7854 | 109.6 | 94.9 | 11.3 | 12.4 |
| WV-7855 | 81.9 | 82.8 | 7.4 | 5.4 |
| WV-7856 | 86.3 | 82.0 | 11.5 | 11.2 |
| WV-7857 | 95.3 | 78.1 | 14.6 | 15.6 |
| WV-7858 | 63.0 | 66.3 | 8.6 | 9.8 |
| WV-7859 | 65.4 | 61.5 | 12.9 | 15.9 |
| WV-7860 | 69.4 | 70.0 | 30.4 | 34.2 |
| WV-7861 | 51.9 | 49.0 | 14.8 | 26.8 |
| WV-7862 | 37.4 | 41.3 | 10.4 | 11.2 |

As shown in Table 90F, WV-993, WV-3390, and WV-4054 were tested.

TABLE 90F
Activity of oligonucleotides.

| | |
|---|---|
| Hep3B | Huh7 |

| mock | 100.0 | 100.0 | 100.0 | 100.0 |
|---|---|---|---|---|
| UT (untreated) | 92.4 | 98.7 | 93.1 | 107.0 |
| WV-993 10 nM | 108.0 | 117.8 | 115.8 | 137.6 |
| WV-3390 2 nM | 84.5 | 84.1 | 46.5 | 57.6 |
| WV-3390 10 nM | 50.0 | 53.9 | 15.7 | 24.4 |
| WV-4054 2 nM | 95.8 | 95.1 | 30.0 | 35.7 |
| WV-4054 10 nM | 85.5 | 91.8 | 30.2 | 37.5 |

As shown in Table 90G, WV-3860 to WV-3864 were tested. Oligonucleotides had mismatches (between wildtype and mutant alleles) at positions 8 and 11 (WV-3860); 9 and 12 (WV-3861); 10 and 13 (WV-3862); 11 and 14 (WV-3863); and 12 and 15 (WV-3864). Several oligonucleotides demonstrated allele-specific knockdown of PNPLA3, particularly at a concentration of 8 nM.

TABLE 90G
Activity of oligonucleotides.

| nM | WV-3860 Hep3B | | WV-3861 Hep3B | | WV-3862 Hep3B | | WV-3863 Hep3B | |
|---|---|---|---|---|---|---|---|---|
| 50 | 37.7 | 44.4 | 44.3 | 47.7 | 21.9 | 32.3 | 32.0 | 32.8 |
| 20 | 83.9 | 89.7 | 87.7 | 93.9 | 64.7 | 72.1 | 81.7 | 87.3 |
| 8 | 95.8 | 95.4 | 101.9 | 103.4 | 90.8 | 92.0 | 84.3 | 93.9 |
| 3.2 | 105.9 | 93.0 | 94.3 | 90.7 | 98.1 | 98.3 | 88.3 | 86.8 |
| 1.28 | 100.9 | 93.4 | 81.0 | 95.4 | 91.0 | 92.7 | 90.9 | 87.4 |
| 0.512 | 90.5 | 96.6 | 94.9 | 88.3 | 92.5 | 93.3 | 87.1 | 88.7 |
| 0.205 | 110.1 | 99.0 | 93.2 | 95.4 | 95.7 | 89.5 | 89.7 | 92.5 |
| 0.082 | 96.6 | 95.6 | 94.8 | 96.2 | 95.3 | 97.8 | 86.7 | 93.9 |
| 0.033 | 98.5 | 88.1 | 98.0 | 95.3 | 103.7 | 93.2 | 93.3 | 87.8 |
| 0.013 | 97.1 | 95.6 | 91.1 | 92.4 | 100.9 | 90.9 | 91.7 | 90.9 |
| nM | WV-3864 Hep3B | | WV-3860 Huh7 | | WV-3861 Huh7 | | WV-3862 Huh7 | |
| 50 | 31.8 | 43.9 | 11.4 | 9.3 | 12.4 | 8.4 | 6.8 | 8.4 |
| 20 | 98.4 | 99.4 | 23.7 | 25.9 | 25.9 | 24.4 | 16.8 | 14.0 |
| 8 | 100.7 | 107.4 | 47.9 | 51.3 | 46.0 | 44.0 | 40.7 | 42.6 |
| 3.2 | 94.9 | 102.7 | 84.1 | 67.6 | 63.7 | 68.7 | 56.9 | 77.6 |
| 1.28 | 96.3 | 95.6 | 100.5 | 83.9 | 80.4 | 83.7 | 77.3 | 81.0 |
| 0.512 | 89.7 | 102.4 | 100.5 | 97.3 | 85.9 | 87.5 | 78.0 | 85.6 |
| 0.205 | 96.3 | 93.8 | 101.6 | 99.9 | 83.7 | 76.6 | 73.4 | 85.7 |
| 0.082 | 89.5 | 92.1 | 81.4 | 87.3 | 85.3 | 84.1 | 78.9 | 89.3 |
| 0.033 | 92.7 | 94.2 | 114.4 | 90.6 | 100.4 | 87.6 | 87.5 | 84.6 |
| 0.013 | 91.7 | 106.0 | 107.0 | 93.7 | 82.3 | 88.1 | 78.3 | 91.6 |

| nM | WV-3863 Huh7 | | WV-3864 Huh7 | |
|---|---|---|---|---|
| 50 | 5.6 | 10.6 | 8.4 | 8.3 |
| 20 | 20.0 | 13.4 | 16.5 | 13.9 |
| 8 | 37.4 | 40.8 | 47.9 | 43.3 |
| 3.2 | 67.5 | 60.5 | 75.6 | 65.5 |
| 1.28 | 84.9 | 82.5 | 86.2 | 87.0 |
| 0.512 | 79.4 | 81.1 | 95.6 | 91.0 |
| 0.205 | 83.0 | 86.3 | 89.1 | 96.1 |
| 0.082 | 79.5 | 92.5 | 89.4 | 77.9 |
| 0.033 | 79.7 | 102.1 | 104.6 | 86.4 |
| 0.013 | 94.6 | 96.8 | 93.0 | 104.9 |

As shown in Table 90H, WV-7804 to WV-7808 were tested. Oligonucleotides had mismatches at positions 8 and 11 (WV-7804); 9 and 12 (WV-7805); 10 and 13 (WV-7806); 11 and 14 (WV-7807); and 12 and 15 (WV-7808). Some oligonucleotides demonstrated allele-specific knockdown of PNPLA3, particularly at concentrations of 3.2 and 8 nM.

TABLE 90H
Activity of oligonucleotides.

| nM | WV-7804 Hep3B | | WV-7805 Hep3B | | WV-7806 Hep3B | | WV-7807 Hep3B | |
|---|---|---|---|---|---|---|---|---|
| 50 | 63.3 | 69.5 | 60.3 | 62.7 | 69.6 | 65.1 | 75.0 | 75.5 |
| 20 | 84.6 | 98.4 | 81.8 | 86.1 | 90.1 | 89.4 | 95.3 | 95.4 |
| 8 | 101.1 | 102.9 | 96.9 | 98.5 | 100.8 | 94.7 | 101.2 | 95.5 |
| 3.2 | 92.9 | 95.1 | 90.7 | 98.1 | 98.9 | 92.1 | | 88.8 |
| 1.28 | 95.5 | 98.6 | 91.3 | 97.0 | 95.9 | 90.9 | 97.3 | 103.6 |
| 0.512 | 96.2 | 110.4 | 95.5 | 97.9 | 95.9 | 94.8 | 100.9 | 92.7 |
| 0.205 | 92.0 | 99.5 | 90.5 | 100.7 | 99.3 | 94.7 | 96.4 | 99.8 |
| 0.082 | 97.6 | 93.6 | 92.2 | 107.6 | 92.3 | 93.8 | 93.4 | 103.7 |
| 0.033 | 98.4 | 101.4 | 98.5 | 104.0 | 99.6 | 90.1 | 89.2 | 93.3 |
| 0.013 | 96.7 | 100.4 | 95.0 | 105.8 | 90.1 | 97.5 | 90.6 | 87.5 |

| nM | WV-7808 Hep3B | WV-7804 Huh7 | | WV-7805 Huh7 | | WV-7806 Huh7 | |
|---|---|---|---|---|---|---|---|
| 50 | 72.2 | 4.4 | 7.5 | 1.7 | 8.4 | 9.8 | 3.8 |
| 20 | 98.9 | 3.5 | 11.1 | 7.1 | 11.9 | 2.8 | 7.2 |
| 8 | 117.8 | 25.1 | 23.3 | 19.6 | 11.6 | 13.8 | 12.5 |
| 3.2 | 109.5 | 45.3 | 48.0 | 36.1 | 38.4 | 28.8 | 39.7 |
| 1.28 | 116.3 | 68.9 | 77.1 | 59.2 | 76.2 | 68.7 | 68.5 |
| 0.512 | 110.9 | 74.6 | 76.0 | 75.3 | 78.4 | 66.6 | 82.4 |
| 0.205 | 109.3 | 73.6 | 86.5 | 69.8 | 81.1 | 69.7 | 80.3 |
| 0.082 | 116.8 | 82.9 | 89.4 | 75.0 | 86.9 | 78.6 | 79.7 |
| 0.033 | 111.1 | 81.8 | 96.1 | 78.0 | 95.8 | 89.4 | 87.3 |
| 0.013 | 104.6 | 86.7 | 90.4 | 79.6 | 95.0 | 88.6 | 98.0 |

| nM | WV-7807 Huh7 | | WV-7808 Huh7 | |
|---|---|---|---|---|
| 50 | 3.0 | 3.9 | 1.8 | 1.4 |
| 20 | 12.6 | 9.1 | 1.9 | 5.2 |
| 8 | 25.1 | 14.9 | 14.2 | 9.1 |
| 3.2 | 46.9 | 41.5 | 37.1 | 33.2 |
| 1.28 | 78.0 | 70.4 | 63.6 | 65.2 |
| 0.512 | 85.3 | 73.7 | 71.0 | 77.4 |
| 0.205 | 98.8 | 68.5 | 79.2 | 92.3 |
| 0.082 | 92.6 | 75.3 | 88.0 | 71.2 |
| 0.033 | 85.6 | 81.9 | 84.1 | 83.5 |
| 0.013 | 85.5 | 94.7 | 95.3 | 78.1 |

As shown in Table 901, WV-7827 to WV-7831 were tested. Oligonucleotides had mismatches at positions 8 and 11 (WV-7827); 9 and 12 (WV-7828); 10 and 13 (WV-7829); 11 and 14 (WV-7830); and 12 and 15 (WV-7831). Several oligonucleotides demonstrated allele-specific knockdown of PNPLA3, particularly at concentrations of 3.2 and 8 nM.

TABLE 90I
Activity of oligonucleotides.

| nM | WV-7827 Hep3B | | WV-7828 Hep3B | | WV-7829 Hep3B | | WV-7830 Hep3B | |
|---|---|---|---|---|---|---|---|---|
| 50 | 37.01 | 34.29 | 36.95 | 32.75 | 25.05 | 33.48 | 36.76 | 54.22 |
| 20 | 79.06 | 85.05 | 85.88 | 77.16 | 82.21 | 79.79 | 79.33 | 87.62 |
| 8 | 97.28 | 89.87 | 93.46 | 91.76 | 99.65 | 98.83 | 97.94 | 97.83 |
| 3.2 | 94.15 | 89.12 | 97.63 | 90.63 | 92.57 | 92.37 | | 104.59 |
| 1.28 | 97.02 | 95.4 | 94.2 | 88.81 | 93.55 | 94.13 | 100.19 | 113.12 |
| 0.512 | 105.31 | 93.06 | 97.26 | 96.7 | 95.81 | 104.04 | 107.33 | 109.89 |
| 0.205 | 107.16 | 97.39 | 92 | 91.65 | 103.76 | 102.33 | 104.56 | 100.78 |
| 0.082 | 100.92 | 101.71 | 94.43 | 88.97 | 93.95 | 105.59 | 94.95 | 110.09 |
| 0.033 | 98.82 | 95.76 | 92.83 | 92.84 | 91.46 | 103.11 | 97.7 | 93.54 |
| 0.013 | 96.78 | 93.38 | 92.91 | 90.28 | 86.25 | 104.33 | 96.73 | 98.6 |

| nM | WV-7831 Hep3B | | WV-7827 Huh7 | | WV-7828 Huh7 | | WV-7829 Huh7 | |
|---|---|---|---|---|---|---|---|---|
| 50 | 53.44 | 62.49 | 3.75 | 3.01 | 2.29 | 1.48 | 5.61 | 7.19 |
| 20 | 97.35 | 92.9 | 6.33 | 5.79 | 16.31 | 9.08 | 16.41 | 9.26 |
| 8 | 103.88 | 103.69 | 13.45 | 13.44 | 9.77 | 24.96 | 31.07 | 18.62 |
| 3.2 | 104.8 | 99.48 | 34.11 | 44.07 | 31.43 | 31.51 | 54.15 | 31.49 |
| 1.28 | 99.77 | 102.88 | 57.91 | 67.23 | 63.32 | 69.34 | 78.28 | 65.69 |
| 0.512 | 102.56 | 99.81 | 80.58 | 87.32 | 83.18 | 75.78 | 94.09 | 75.96 |
| 0.205 | 111.83 | 98.89 | 71.87 | 84.73 | 74.94 | 76.52 | 87.05 | 90.6 |
| 0.082 | 102.02 | 93.55 | 74.43 | 76.07 | 82.31 | 87.03 | 97.43 | 87.48 |
| 0.033 | 93.54 | 101.84 | 77.64 | 80.62 | 89.1 | 89.96 | 108.41 | 81.93 |
| 0.013 | 101.47 | 100.78 | 77.47 | 74.02 | 83.14 | 79.83 | 100.58 | 88.97 |

| nM | WV-7830 Huh7 | | WV-7831 Huh7 | |
|---|---|---|---|---|
| 50 | 4.11 | 5.5 | 3.5 | 5.45 |
| 20 | 6.32 | 5.86 | 5.62 | 10.22 |
| 8 | 12.5 | 16.99 | 15.15 | 23.97 |
| 3.2 | 35.34 | 40.2 | 35 | 40.3 |
| 1.28 | 56.82 | 76.27 | 73.64 | 73.96 |
| 0.512 | 86.38 | 77.53 | 83.87 | 88.08 |
| 0.205 | 80.08 | 79.81 | 85.02 | 86.98 |
| 0.082 | 95.69 | 98.61 | 82.31 | 111.77 |
| 0.033 | 86.65 | 93.2 | 85.86 | 85.82 |
| 0.013 | 94.2 | 85.75 | 81.84 | 93.28 |

As shown in Table 90J, WV-993 (negative control), WV-3390 (positive control), WV-4054, WV-7850 to WV-7854 were tested. The oligonucleotides had mismatches at positions 8 and 11 (WV-7850); 9 and 12 (WV-7851); 10 and 13 (WV-7852); 11 and 14 (WV-7853); and 12 and 15 (WV-7854). Several oligonucleotides demonstrated high allele-specific knockdown of PNPLA3, particularly at concentrations of 3.2 and 8 nM.

TABLE 90J
Activity of oligonucleotides.

| nM | WV-7850 Hep3B | | WV-7851 Hep3B | | WV-7852 Hep3B | | WV-7853 Hep3B | |
|---|---|---|---|---|---|---|---|---|
| 50 | 38.95 | 43.11 | 38.2 | 54.32 | 34.89 | 44.6 | 28.33 | 29.79 |
| 20 | 65.77 | 72.48 | 80.99 | 84.09 | 69.74 | 74.87 | 66.38 | 50.49 |
| 8 | 92.95 | 82.91 | 93.58 | 96.06 | 89.54 | 101.29 | 85.15 | 81.31 |
| 3.2 | 91.26 | 86.61 | 90.46 | 94.37 | 97.29 | 91.77 | | 96.08 |
| 1.28 | 111.06 | 94.39 | 98.64 | 92.99 | 89.98 | 111.31 | 96.26 | 97.11 |
| 0.512 | 106.14 | 87.28 | 94.61 | 94.09 | 96.79 | 95.82 | 109.19 | 105.33 |
| 0.205 | 84.62 | 87.72 | 100.09 | 99.5 | 111.54 | 99.81 | 95.06 | 94.02 |
| 0.082 | 97.3 | 89.13 | 90.19 | 87.13 | 92.11 | 100.64 | 95.93 | 104.77 |
| 0.033 | 88.83 | 89.09 | 96.09 | 101.92 | 96.1 | 95.83 | 95.56 | 95.83 |
| 0.013 | 91.98 | 92.51 | 100.59 | 92.81 | 98.94 | 109.53 | 99.05 | 97.36 |

| nM | WV-7854 Hep3B | | WV-7850 Huh7 | | WV-7851 Huh7 | | WV-7852 Huh7 | |
|---|---|---|---|---|---|---|---|---|
| 50 | 16.7 | 18.84 | 2.74 | 4.3 | 2.13 | 1.26 | 8.39 | 7.43 |
| 20 | 63.04 | 59.9 | 5.33 | 5.88 | 6.09 | 4.64 | 7.76 | 5.84 |
| 8 | 82.27 | 80.67 | 11.67 | 9.74 | 1.12 | 14.86 | 9.96 | 10.6 |
| 3.2 | 92.77 | 89.43 | 25.14 | 13.84 | 31.45 | 25.89 | 28.6 | 25.4 |
| 1.28 | 90.63 | 83.96 | 57.25 | 62.49 | 55.19 | 52.3 | 49.96 | 56.13 |
| 0.512 | 88.27 | 86.56 | 75.21 | 73.3 | 81.73 | 81.67 | 72.77 | 87.64 |
| 0.205 | 89.69 | 88.92 | 71.16 | 74.28 | 91.84 | 94.95 | 82.61 | 85.11 |
| 0.082 | 95.16 | 89.43 | 84.71 | 75.77 | 91 | 79.62 | 96.22 | 83.67 |
| 0.033 | 99.41 | 94.3 | 80.67 | 93.66 | 84.27 | 79.5 | 72.16 | 70.12 |
| 0.013 | 88 | 96.49 | 96.84 | 104.2 | 86.94 | 81.37 | 94.13 | 88.49 |

| nM | WV-7853 Huh7 | | WV-7854 Huh7 | |
|---|---|---|---|---|
| 50 | 2.91 | 3.83 | 0.56 | 5.35 |
| 20 | 5.38 | 4.3 | 2.43 | 7.48 |
| 8 | 6.36 | 8.51 | 9.91 | 10.31 |
| 3.2 | 27.05 | 23.79 | 23.52 | 22.24 |
| 1.28 | 47.78 | 62.17 | 47.66 | 45.94 |
| 0.512 | 70.13 | 93.78 | 76.63 | 66.5 |
| 0.205 | 96.01 | 74.6 | 84.06 | 72.69 |
| 0.082 | 82.68 | 90.37 | 79.33 | 82.72 |
| 0.033 | 89.38 | 93.35 | 89.39 | 83.68 |
| 0.013 | 86.29 | 95.77 | 98.79 | 96.55 |

As shown in Table 90K and 90L, WV-3860 to WV-3864, WV-7804 to WV-7808, WV-7827 to WV-7831, and WV-7850 to WV-7854 were tested. WV-4054 demonstrated high allele-specific activity.

TABLE 90K
Activity of oligonucleotides.

| | | |
|---|---|---|
| nM | WV-3390 Hep3B | WV-3390 Huh7 |

| | | | | |
|---|---|---|---|---|
| 50 | 8.21 | 7.25 | 8.29 | 8.93 |
| 20 | 6.38 | 7.65 | 8.38 | 6.55 |
| 8 | 12.25 | 18.67 | 14.29 | 10.45 |
| 3.2 | 45.11 | 53.94 | 44.3 | 57.9 |
| 1.28 | 77.33 | 87.7 | 75.75 | 79.58 |
| 0.512 | 89.66 | 91.59 | 75.55 | 114.48 |
| 0.205 | 88.47 | 99.01 | 77.44 | 93.14 |
| 0.082 | 90.52 | 101.67 | 81.19 | 91 |
| 0.033 | 100.47 | 102.34 | 78.08 | 106.05 |
| 0.013 | 111.98 | 101.98 | 93.49 | 92.23 |

| | | |
|---|---|---|
| nM | WV-4054 Hep3B | WV-4054 Huh7 |

| | | | | |
|---|---|---|---|---|
| 8 | 77.1 | 75.46 | 25.4 | 28.99 |
| 3.2 | 64.83 | 77.36 | 19.76 | 30.45 |
| 1.28 | 75.7 | 80.58 | 20.26 | 28 |
| 0.512 | 83.19 | 91.18 | 27.99 | 30.12 |
| 0.205 | 96.79 | 100.8 | 48.79 | 51.24 |
| 0.082 | 96.97 | 105.15 | 51.71 | 65.92 |
| 0.033 | 98.21 | 96.76 | 82.73 | 90.69 |
| 0.013 | 100.71 | 110.73 | 89.25 | 101.52 |

| | | |
|---|---|---|
| nM | WV-993 Hep3B | WV-993 Huh7 |

| | | | | |
|---|---|---|---|---|
| 50 | 46.96 | 63.05 | 64.33 | 84.69 |
| 20 | 85.99 | 93.33 | 85.91 | 92.58 |
| 8 | 94.55 | 112.34 | 108.31 | 90.19 |
| 3.2 | 94.42 | 101.21 | 98.4 | 102.07 |
| 1.28 | 90.96 | 98.52 | 104.92 | 108.34 |
| 0.512 | 87.71 | 95.84 | 96.98 | 87.06 |
| 0.205 | 91.78 | 93.62 | 114.92 | 90.64 |
| 0.082 | 86.49 | 96.33 | 109.17 | 101.77 |
| 0.033 | 87.45 | 104.34 | 102.65 | 92.43 |
| 0.013 | 90.55 | 100.97 | 114.84 | 108.07 |

TABLE 90L
IC50 of various oligonucleotides in Huh7 cells (mutant allele).

| Mismatch | Oligonucleotides | IC₅₀ | Oligonucleotides | IC₅₀ |
|---|---|---|---|---|
| Positions | 2′OMe | (nM) | 2′MOE | (nM) |
| 8, 11 | WV-3860 | 10.6 | WV-7804 | 4.0 |
| 9, 12 | WV-3861 | 10.4 | WV-7805 | 3.0 |
| 10, 13 | WV-3862 | 10.6 | WV-7806 | 2.6 |
| 11, 14 | WV-3863 | 7.8 | WV-7807 | 4.0 |
| 12, 15 | WV-3864 | 9.5 | WV-7808 | 2.9 |
| Mismatch | Oligonucleotides | IC₅₀ | Oligonucleotides | IC₅₀ |
| Positions | 2′MOE LNA | (nM) | 2′OMe LNA | (nM) |
| 8, 11 | WV-7850 | 1.6 | WV-7827 | 3.8 |
| 9, 12 | WV-7851 | 2.2 | WV-7828 | 2.8 |
| 10, 13 | WV-7852 | 1.9 | WV-7829 | 3.0 |
| 11, 14 | WV-7853 | 1.8 | WV-7830 | 2.6 |
| 12, 15 | WV-7854 | 1.3 | WV-7831 | 3.3 |

TABLE 91
Activity of oligonucleotides.
PNPLA3 mRNA level
(PNPLA3/HPRT1)

| | Conc (nM) exp 10 | | | | | | | |
|---|---|---|---|---|---|---|---|---|
| | 1.477 | 1.079 | 0.681 | 0.283 | −0.114 | −0.512 | −0.910 | −1.308 |
| WV-7808 | 0.200 | 0.366 | 0.424 | 0.576 | 0.803 | 0.910 | 1.217 | 1.131 |
| | 0.081 | 0.221 | 0.374 | 0.613 | 0.987 | 1.050 | 1.314 | 1.080 |
| WV-8690 | 0.199 | 0.388 | 0.503 | 0.808 | 0.897 | 1.000 | 1.148 | 1.068 |
| | 0.208 | 0.313 | 0.754 | 0.932 | 1.058 | 1.236 | 1.286 | 1.138 |
| WV-8858 | 0.233 | 0.446 | 0.595 | 0.838 | 0.911 | 0.916 | 0.965 | 1.213 |
| | 0.240 | 0.340 | 0.727 | 1.036 | 1.039 | 1.403 | 0.874 | |
| WV-8859 | 0.086 | 0.292 | 0.279 | 0.710 | 0.850 | 1.071 | 0.956 | 1.091 |
| | 0.083 | 0.217 | 0.505 | 0.754 | 0.981 | 1.258 | 1.131 | 1.454 |
| WV-8860 | 0.234 | 0.386 | 0.385 | 0.751 | 0.867 | 0.947 | 1.358 | 1.057 |
| | 0.162 | 0.321 | 0.503 | 1.002 | 1.100 | 1.075 | 1.250 | 1.241 |

TABLE 92
Activity of oligonucleotides.

| | 1.477 | 1.079 | 0.681 | 0.283 | −0.115 | −0.513 | −0.911 | −1.308 |
|---|---|---|---|---|---|---|---|---|
| WV-7807 | 0.102 | 0.331 | 0.588 | 0.790 | 1.037 | 0.989 | 1.271 | 1.147 |
| | 0.137 | 0.397 | 0.453 | 0.814 | 1.160 | 1.099 | 1.257 | 1.027 |
| WV-8854 | 0.117 | 0.375 | 0.678 | 0.831 | 0.962 | 1.021 | 1.258 | 1.277 |
| | 0.112 | 0.363 | 0.559 | 0.793 | 1.134 | 1.237 | 1.226 | 1.186 |
| WV-8855 | 0.174 | 0.553 | 0.745 | 0.873 | 0.890 | 0.968 | 0.954 | 1.088 |
| | 0.181 | 0.462 | 0.690 | 0.737 | 1.102 | 1.168 | 0.930 | 1.006 |
| WV-8856 | 0.055 | 0.239 | 0.496 | 0.779 | 0.815 | 0.937 | 1.029 | 1.027 |
| | 0.069 | 0.288 | 0.654 | 0.884 | 1.172 | 1.146 | 0.937 | 1.222 |
| WV-8857 | 0.237 | 0.445 | 0.967 | 0.928 | 0.976 | 0.790 | 0.992 | 1.119 |
| | 0.188 | 0.504 | 0.783 | 0.932 | 1.031 | 1.046 | 1.086 | 1.067 |

TABLE 93
Activity of oligonucleotides.

| | 1.477 | 1.079 | 0.681 | 0.283 | −0.114 | −0.512 | −0.910 | −1.308 |
|---|---|---|---|---|---|---|---|---|
| WV-7806 | 0.15 | 0.24 | 0.29 | 0.71 | 0.92 | 1.03 | 1.02 | 1.10 |
| | 0.14 | 0.16 | 0.30 | 0.56 | 0.67 | 0.84 | 1.02 | 0.89 |
| WV-8850 | 0.57 | 0.61 | 0.59 | 1.09 | 1.08 | 1.04 | 1.11 | 1.29 |
| | 0.44 | 0.52 | 0.60 | 0.65 | 0.81 | 0.76 | 0.96 | 0.93 |
| WV-8851 | 0.18 | 0.41 | 0.43 | 0.95 | 0.93 | 1.00 | 1.08 | 1.05 |
| | 0.17 | 0.27 | 0.63 | 0.71 | 0.70 | 1.01 | 0.90 | 0.85 |
| WV-8852 | 0.55 | 0.29 | 0.32 | 0.83 | 1.04 | 1.23 | 0.90 | 1.28 |
| | 0.14 | 0.18 | 0.26 | 0.49 | 0.77 | 0.83 | 1.09 | 0.92 |
| WV-8853 | 0.21 | 0.38 | 0.41 | 0.76 | 1.24 | 0.92 | 1.08 | 0.93 |
| | 0.13 | 0.20 | 0.44 | 0.61 | 0.94 | 0.64 | 0.87 | 0.95 |

TABLE 94
Activity of oligonucleotides.

| | 1.477 | 1.079 | 0.681 | 0.283 | −0.114 | −0.512 | −0.910 | −1.308 |
|---|---|---|---|---|---|---|---|---|
| WV-7805 | 0.29 | 0.33 | 0.44 | 0.72 | 0.95 | 0.96 | 0.93 | 0.99 |
| | 0.15 | 0.27 | 0.41 | 0.73 | 1.12 | 1.19 | 0.78 | 0.87 |
| WV-8609 | 0.33 | 0.37 | 0.48 | 0.90 | 0.84 | 0.81 | 1.02 | 1.04 |
| | 0.13 | 0.29 | 0.56 | 0.76 | 0.89 | 1.15 | 1.07 | 0.91 |
| WV-8847 | 0.24 | 0.37 | 0.58 | 0.78 | 0.89 | 1.20 | 0.91 | 0.97 |
| | 0.14 | 0.23 | 0.64 | 0.79 | 0.90 | 1.16 | 0.94 | 1.19 |
| WV-8848 | 0.19 | 0.32 | 0.47 | 0.69 | 0.93 | 0.88 | 0.92 | 0.92 |
| | 0.19 | 0.16 | 0.46 | 0.74 | 0.88 | 0.79 | 1.03 | 1.09 |
| WV-8849 | 0.24 | 0.39 | 0.55 | 0.79 | 1.17 | 0.97 | 1.16 | 0.95 |
| | 0.28 | 0.29 | 0.54 | 0.82 | 1.05 | 1.13 | 1.17 | 1.05 |

TABLE 95

| | 1.477 | 1.079 | 0.681 | 0.283 | −0.114 | −0.512 | −0.910 | −1.308 |
|---|---|---|---|---|---|---|---|---|
| WV-7804 | 0.29 | 0.37 | 0.51 | 0.81 | 0.85 | 1.16 | 0.87 | 0.89 |
| | 0.19 | 0.22 | 0.47 | 0.82 | 0.85 | 0.94 | 1.05 | 1.06 |
| WV-8843 | 0.53 | 0.72 | 0.62 | 1.00 | 0.98 | 0.85 | 0.92 | 0.98 |
| | 0.45 | 0.51 | 0.61 | 0.93 | 0.93 | 1.01 | 0.90 | 1.01 |
| WV-8844 | 0.25 | 0.44 | 0.58 | 0.78 | 0.71 | 0.86 | 0.86 | 1.00 |
| | 0.22 | 0.21 | 0.48 | 0.76 | 1.02 | 1.06 | 0.74 | 1.16 |
| WV-8845 | 0.23 | 0.42 | 0.52 | 0.82 | 0.99 | 0.87 | 0.77 | 1.11 |
| | 0.17 | 0.25 | 0.44 | 0.76 | 0.90 | 0.97 | 0.99 | 0.88 |
| WV-8846 | 0.20 | 0.38 | 0.55 | 0.60 | 0.90 | 0.76 | 0.88 | 0.98 |
| | 0.17 | 0.25 | 0.39 | 0.71 | 1.11 | 0.92 | 0.83 | 1.04 |

TABLE 98
Activity of oligonucleotides.
10 nM.

| | |
|---|---|
| Hep3B | Huh7 |

| mock | 100 | 100 | 100 | 100 |
|---|---|---|---|---|
| WV-7794 | 47.6 | 44.5 | 25.0 | 20.6 |
| WV-7795 | 58.5 | 52.8 | 14.3 | 14.8 |
| WV-7796 | 54.6 | 56.4 | 16.3 | 15.5 |
| WV-7797 | 75.1 | 74.2 | 13.5 | 12.8 |
| WV-7798 | 78.4 | 79.8 | 11.9 | 13.7 |
| WV-7799 | 89.9 | 92.4 | 23.5 | 25.7 |
| WV-7800 | 93.6 | 92.2 | 34.1 | 29.9 |
| WV-7801 | 90.3 | 90.3 | 38.4 | 29.3 |
| WV-7802 | 101.1 | 101.3 | 25.1 | 29.6 |
| WV-7803 | 102.0 | 103.2 | 24.8 | 25.8 |
| WV-7804 | 95.9 | 97.2 | 27.8 | 32.7 |
| WV-7805 | 100.5 | 95.5 | 21.9 | 22.0 |
| WV-7806 | 110.6 | 105.4 | 22.0 | 21.2 |
| WV-7807 | 96.2 | 101.5 | 21.1 | 23.8 |
| WV-7808 | 95.5 | 101.0 | 21.5 | 18.8 |
| WV-7809 | 85.3 | 84.2 | 17.1 | 15.7 |
| WV-7810 | 92.0 | 95.9 | 25.2 | 21.6 |
| WV-7811 | 100.1 | 100.0 | 26.6 | 27.1 |
| WV-7812 | 79.5 | 82.1 | 22.3 | 21.1 |
| WV-7813 | 83.7 | 76.2 | 23.7 | 18.2 |
| WV-7814 | 87.8 | 82.8 | 44.3 | 39.2 |
| WV-7815 | 78.1 | 74.8 | 45.3 | 37.1 |
| WV-7816 | 59.5 | 52.4 | 24.5 | 20.5 |

TABLE 99
Activity of oligonucleotides.
10 nM.

| | |
|---|---|
| Hep3B | Huh7 |

| mock | 100 | 100 | 100 | 100 |
|---|---|---|---|---|
| WV-7778 | 92.8 | 86.9 | 63.6 | 52.7 |
| WV-7779 | 92.6 | 85.8 | 59.2 | 48.0 |
| WV-7780 | 102.3 | 97.1 | 36.4 | 34.8 |
| WV-7781 | 111.8 | 102.4 | 46.1 | 38.9 |
| WV-7782 | 108.5 | 94.7 | 43.3 | 35.0 |
| WV-7783 | 110.2 | 97.5 | 36.6 | 33.7 |
| WV-7784 | 105.0 | 95.9 | 41.2 | 41.9 |
| WV-7785 | 108.7 | 107.4 | 73.8 | 57.5 |
| WV-3858 | 103.3 | 103.6 | 55.1 | 52.3 |
| WV-3859 | 94.7 | 96.1 | 41.8 | 41.2 |
| WV-3860 | 104.2 | 94.1 | 46.7 | 45.6 |
| WV-3861 | 101.8 | 99.2 | 47.1 | 45.2 |
| WV-3862 | 97.1 | 96.5 | 44.0 | 42.3 |
| WV-3863 | 99.7 | 86.0 | 42.0 | 51.5 |
| WV-3864 | 97.8 | 96.8 | 53.5 | 38.9 |
| WV-7786 | 99.1 | 104.3 | 37.0 | 38.0 |
| WV-7787 | 84.2 | 87.5 | 23.0 | 25.1 |
| WV-7788 | 80.4 | 88.0 | 35.5 | 28.5 |
| WV-7789 | 82.7 | 85.7 | 25.4 | 25.5 |
| WV-7790 | 80.8 | 85.1 | 27.4 | 29.8 |
| WV-7791 | 87.1 | 80.1 | 40.8 | 42.1 |
| WV-7792 | 78.1 | 73.1 | 42.5 | 46.5 |
| WV-7793 | 68.3 | 65.1 | 49.9 | 44.9 |

TABLE 100
Activity of oligonucleotides.
% mRNA remaining
(RhPNPLA3/hSFRS9)
Monkey hepatocytes at 48 hrs.

| | | |
|---|---|---|
| 10 nM | 3 nM | 1 nM |

| Mock | 100 | 100 | 100 | 100 | 100 | 100 |
|---|---|---|---|---|---|---|
| WV-3421 | 28.7 | 35.4 | 44.6 | 31.8 | 61.3 | 53.8 |
| WV-7794 | 64.1 | 74.4 | 104.3 | 96.2 | 115.5 | 121.3 |
| WV-7795 | 80.8 | 88.4 | 130.2 | 115.9 | 109.0 | 130.0 |
| WV-7796 | 51.3 | 53.4 | 83.8 | 95.5 | 106.9 | 103.3 |
| WV-7797 | 51.4 | 48.5 | 97.3 | 81.4 | 115.1 | 126.7 |
| WV-7798 | 65.2 | 56.8 | 84.8 | 86.8 | 106.8 | 104.0 |
| WV-7799 | 96.5 | 102.5 | 104.7 | 117.6 | 108.9 | 129.6 |
| WV-7800 | 66.1 | 78.8 | 113.3 | 111.3 | 114.9 | 128.1 |
| WV-7801 | 113.1 | 117.7 | 116.0 | 112.7 | 113.3 | 118.5 |
| WV-7802 | 101.6 | 110.7 | 105.8 | 120.3 | 113.3 | 123.9 |
| WV-7803 | 52.5 | 59.6 | 73.6 | 79.4 | 106.5 | 129.4 |
| WV-7804 | 95.5 | 91.3 | 111.3 | 137.4 | 116.5 | 122.1 |
| WV-7805 | 84.7 | 97.8 | 111.7 | 111.1 | 114.3 | 118.7 |
| WV-7806 | 91.2 | 87.2 | 129.7 | 121.3 | 124.8 | 121.8 |
| WV-7807 | 64.5 | 89.2 | 108.5 | 119.7 | 108.6 | 123.7 |
| WV-7808 | 39.4 | 48.1 | 94.7 | 105.8 | 105.1 | 125.5 |
| WV-7809 | 46.5 | 36.8 | 77.0 | 64.9 | 85.1 | 101.2 |
| WV-7810 | 46.7 | 46.5 | 62.1 | 78.5 | 75.8 | 94.7 |
| WV-7811 | 70.4 | 78.7 | 88.2 | 84.0 | 101.1 | 96.6 |
| WV-7812 | 47.2 | 53.3 | 74.5 | 80.7 | 97.7 | 77.8 |
| WV-7813 | 43.0 | 38.3 | 76.5 | 71.2 | 97.0 | 89.7 |
| WV-7814 | 49.8 | 51.2 | 102.8 | 96.1 | 105.9 | 131.8 |
| WV-7815 | 56.0 | 52.5 | 88.3 | 83.8 | 83.4 | 94.9 |
| WV-7816 | 29.3 | 19.7 | 51.5 | 57.5 | 84.4 | 68.4 |

TABLE 101
Activity of oligonucleotides.
PNPLA3 mRNA Level
(PNPLA3/GAPDH)

| | 0.12 nM | 0.4 nM | 1.1. nM |
|---|---|---|---|
| WV-993 | 99.8 | 77.9 | 74.5 |
| WV-3421 | 74.9 | 44.6 | 24.0 |
| WV-7805 | 105.4 | 99.2 | 83.8 |
| WV-9890 | 108.6 | 78.4 | 78.3 |
| WV-12100 | 104.6 | 102.7 | 93.2 |
| WV-9893 | 93.7 | 103.8 | 79.8 |
| WV-12101 | 124.1 | 67.6 | 36.6 |

TABLE 102A
Activity of oligonucleotides.
Primary cyno hepatocytes.

| Conc. (nM) | WV-9893 | | WV-3421 | | WV-12101 | |
|---|---|---|---|---|---|---|
| 1.079181 | 116.7 | 90.2 | 13.6 | 6.5 | 20.1 | 27.6 |
| 0.681241 | 135.5 | 98.9 | 13.9 | 5.4 | 20.1 | |
| 0.283301 | 86.5 | 126.1 | 32.9 | 23.7 | 11.0 | 37.7 |
| −0.11464 | 105.3 | 108.9 | 70.7 | 46.7 | 40.7 | |
| −0.51258 | 121.9 | 114.1 | 89.3 | 81.5 | 70.0 | 97.1 |
| −0.91052 | 112.7 | 137.8 | 124.2 | 113.7 | 81.7 | 114.1 |
| −1.30846 | 116.0 | 110.1 | 134.7 | 80.5 | 81.0 | 72.6 |
| −1.7064 | 120.5 | 106.7 | 105.7 | 140.0 | 82.5 | 77.1 |
| −2.10434 | 120.5 | 108.0 | 131.0 | 95.2 | 98.4 | 88.2 |
| −2.50228 | 94.8 | 99.6 | 89.2 | 85.4 | 106.7 | 89.7 |

TABLE 102B
Tm of oligonucleotides.

| | | Duplex | Duplex | Δ difference |
|---|---|---|---|---|
| | | Tm(° C.) | Tm(° C.) | Full match |
| | | WV-12420 | WV-12421 Two | vs two |
| ASO | Length | Full match | mismatches | mismatches |
| WV-7805 | 20-mer | 63.52 | 47.62 | 15.9 |
| WV-9891 | 20-mer | 61.62 | 44.77 | 16.9 |
| WV-9890 | 20-mer | 61.57 | 46.67 | 14.9 |
| WV-9893 | 20-mer | 58.67 | 43.52 | 15.2 |
| WV-12106 | 24-mer | 71.52 | 59.72 | 11.8 |
| WV-12107 | 24-mer | 69.57 | 57.77 | 11.8 |
| WV-12100 | 24-mer | 70.77 | 59.57 | 11.2 |
| WV-12101 | 24-mer | 67.52 | 56.62 | 10.9 |

The Tm of various oligonucleotides was measured while in duplex with a RNA which was completely complementary, or which was completely complementary except for two mismatches (representing the mutant allele). Conditions used were: 1 μM Duplex in 1×PBS (pH 7.2); Temperature Range: 15° C.-90° C.; Temperature Rate: 0.5° C./min; Measurement Interval: 0.5° C.

TABLE 103
Activity of oligonucleotides.
Huh7 cells.

| Conc. (nM) | 1 | 0.52288 | 0.04576 | −0.4314 | −0.9085 | −1.3856 | −1.8627 |
|---|---|---|---|---|---|---|---|
| WV-7805 | 7.0 | 23.5 | 64.7 | 80.4 | 88.2 | 92.5 | 99.5 |
| | 5.1 | 24.7 | 78.9 | 74.1 | 86.7 | | |
| WV-9890 | 1.6 | 35.0 | | 90.4 | 90.3 | 92.6 | 104.6 |
| | 13.1 | 29.2 | 73.7 | 88.5 | 87.1 | 95.8 | 105.5 |
| WV-12100 | 12.4 | 33.8 | 63.6 | 90.6 | | 102.3 | 101.5 |
| | 10.2 | 27.6 | 76.6 | 80.0 | 83.6 | 80.7 | 85.0 |
| WV-9893 | 10.4 | 28.7 | 74.3 | 86.3 | 87.7 | 116.1 | 93.8 |
| | 4.3 | 36.4 | 80.4 | 91.5 | 110.3 | 108.6 | 106.5 |
| WV-12101 | | 4.6 | 19.8 | 60.3 | 85.8 | 92.0 | 108.3 |
| | | 6.8 | 19.2 | 60.0 | 81.1 | 81.0 | 85.6 |

TABLE 104
Activity of oligonucleotides.
nM

| Conc. (nM) | 50 | 20 | 8 | 3.2 | 1.28 | 0.51 | 0.20 | 0.081 | 0.032 | 0.013 |
|---|---|---|---|---|---|---|---|---|---|---|
| WV-7850 | 39.0 | 65.8 | 93.0 | 91.3 | 111.1 | 106.1 | 84.6 | 97.3 | 88.8 | 92.0 |
| Hep3B | 43.1 | 72.5 | 82.9 | 86.6 | 94.4 | 87.3 | 87.7 | 89.1 | 89.1 | 92.5 |
| WV-7851 | 38.2 | 81.0 | 93.6 | 90.5 | 98.6 | 94.6 | 100.1 | 90.2 | 96.1 | 100.6 |
| Hep3B | 54.3 | 84.1 | 96.1 | 94.4 | 93.0 | 94.1 | 99.5 | 87.1 | 101.9 | 92.8 |
| WV-7852 | 34.9 | 69.7 | 89.5 | 97.3 | 90.0 | 96.8 | 111.5 | 92.1 | 96.1 | 98.9 |
| Hep3B | 44.6 | 74.9 | 101.3 | 91.8 | 111.3 | 95.8 | 99.8 | 100.6 | 95.8 | 109.5 |
| WV-7853 | 28.3 | 66.4 | 85.2 | | 96.3 | 109.2 | 95.1 | 95.9 | 95.6 | 99.1 |
| Hep3B | 29.8 | 50.5 | 81.3 | 96.1 | 97.1 | 105.3 | 94.0 | 104.8 | 95.8 | 97.4 |
| WV-7854 | 16.7 | 63.0 | 82.3 | 92.8 | 90.6 | 88.3 | 89.7 | 95.2 | 99.4 | 88.0 |
| Hep3B | 18.8 | 59.9 | 80.7 | 89.4 | 84.0 | 86.6 | 88.9 | 89.4 | 94.3 | 96.5 |
| WV-7850 | 2.7 | 5.3 | 11.7 | 25.1 | 57.3 | 75.2 | 71.2 | 84.7 | 80.7 | 96.8 |
| Huh7 | 4.3 | 5.9 | 9.7 | 13.8 | 62.5 | 73.3 | 74.3 | 75.8 | 93.7 | 104.2 |
| WV-7851 | 2.1 | 6.1 | 1.1 | 31.5 | 55.2 | 81.7 | 91.8 | 91.0 | 84.3 | 86.9 |
| Huh7 | 1.3 | 4.6 | 14.9 | 25.9 | 52.3 | 81.7 | 95.0 | 79.6 | 79.5 | 81.4 |
| WV-7852 | 8.4 | 7.8 | 10.0 | 28.6 | 50.0 | 72.8 | 82.6 | 96.2 | 72.2 | 94.1 |
| Huh7 | 7.4 | 5.8 | 10.6 | 25.4 | 56.1 | 87.6 | 85.1 | 83.7 | 70.1 | 88.5 |
| WV-7853 | 2.9 | 5.4 | 6.4 | 27.1 | 47.8 | 70.1 | 96.0 | 82.7 | 89.4 | 86.3 |
| Huh7 | 3.8 | 4.3 | 8.5 | 23.8 | 62.2 | 93.8 | 74.6 | 90.4 | 93.4 | 95.8 |
| WV-7854 | 0.6 | 2.4 | 9.9 | 23.5 | 47.7 | 76.6 | 84.1 | 79.3 | 89.4 | 98.8 |
| Huh7 | 5.4 | 7.5 | 10.3 | 22.2 | 45.9 | 66.5 | 72.7 | 82.7 | 83.7 | 96.6 |

TABLE 105
Activity of oligonucleotides.

| nM | 50 | 20 | 8 | 3.2 | 1.28 | 0.51 | 0.204 | 0.081 | 0.032 | 0.013 |
|---|---|---|---|---|---|---|---|---|---|---|
| WV-7827 | 37.0 | 79.1 | 97.3 | 94.2 | 97.0 | 105.3 | 107.2 | 100.9 | 98.8 | 96.8 |
| Hep3B | 34.3 | 85.1 | 89.9 | 89.1 | 95.4 | 93.1 | 97.4 | 101.7 | 95.8 | 93.4 |
| WV-7828 | 37.0 | 85.9 | 93.5 | 97.6 | 94.2 | 97.3 | 92.0 | 94.4 | 92.8 | 92.9 |
| Hep3B | 32.8 | 77.2 | 91.8 | 90.6 | 88.8 | 96.7 | 91.7 | 89.0 | 92.8 | 90.3 |
| WV-7829 | 25.1 | 82.2 | 99.7 | 92.6 | 93.6 | 95.8 | 103.8 | 94.0 | 91.5 | 86.3 |
| Hep3B | 33.5 | 79.8 | 98.8 | 92.4 | 94.1 | 104.0 | 102.3 | 105.6 | 103.1 | 104.3 |
| WV-7830 | 36.8 | 79.3 | 97.9 | | 100.2 | 107.3 | 104.6 | 95.0 | 97.7 | 96.7 |
| Hep3B | 54.2 | 87.6 | 97.8 | 104.6 | 113.1 | 109.9 | 100.8 | 110.1 | 93.5 | 98.6 |
| WV-7831 | 53.4 | 97.4 | 103.9 | 104.8 | 99.8 | 102.6 | 111.8 | 102.0 | 93.5 | 101.5 |
| Hep3B | 62.5 | 92.9 | 103.7 | 99.5 | 102.9 | 99.8 | 98.9 | 93.6 | 101.8 | 100.8 |
| WV-7827 | 3.8 | 6.3 | 13.5 | 34.1 | 57.9 | 80.6 | 71.9 | 74.4 | 77.6 | 77.5 |
| Huh7 | 3.0 | 5.8 | 13.4 | 44.1 | 67.2 | 87.3 | 84.7 | 76.1 | 80.6 | 74.0 |
| WV-7828 | 2.3 | 16.3 | 9.8 | 31.4 | 63.3 | 83.2 | 74.9 | 82.3 | 89.1 | 83.1 |
| Huh7 | 1.5 | 9.1 | 25.0 | 31.5 | 69.3 | 75.8 | 76.5 | 87.0 | 90.0 | 79.8 |
| WV-7829 | 5.6 | 16.4 | 31.1 | 54.2 | 78.3 | 94.1 | 87.1 | 97.4 | 108.4 | 100.6 |
| Huh7 | 7.2 | 9.3 | 18.6 | 31.5 | 65.7 | 76.0 | 90.6 | 87.5 | 81.9 | 89.0 |
| WV-7830 | 4.1 | 6.3 | 12.5 | 35.3 | 56.8 | 86.4 | 80.1 | 95.7 | 86.7 | 94.2 |
| Huh7 | 5.5 | 5.9 | 17.0 | 40.2 | 76.3 | 77.5 | 79.8 | 98.6 | 93.2 | 85.8 |
| WV-7831 | 3.5 | 5.6 | 15.2 | 35.0 | 73.6 | 83.9 | 85.0 | 82.3 | 85.9 | 81.8 |
| Huh7 | 5.5 | 10.2 | 24.0 | 40.3 | 74.0 | 88.1 | 87.0 | 111.8 | 85.8 | 93.3 |

TABLE 106
Activity of oligonucleotides.

| nM | 50 | 20 | 8 | 3.2 | 1.28 | 0.51 | 0.204 | 0.081 | 0.032 | 0.013 |
|---|---|---|---|---|---|---|---|---|---|---|
| WV-7804 | 63.3 | 84.6 | 101.1 | 92.9 | 95.5 | 96.2 | 92.0 | 97.6 | 98.4 | 96.7 |
| Hep3B | 69.5 | 98.4 | 102.9 | 95.1 | 98.6 | 110.4 | 99.5 | 93.6 | 101.4 | 100.4 |
| WV-7805 | 60.3 | 81.8 | 96.9 | 90.7 | 91.3 | 95.5 | 90.5 | 92.2 | 98.5 | 95.0 |
| Hep3B | 62.7 | 86.1 | 98.5 | 98.1 | 97.0 | 97.9 | 100.7 | 107.6 | 104.0 | 105.8 |
| WV-7806 | 69.6 | 90.1 | 100.8 | 98.9 | 95.9 | 95.9 | 99.3 | 92.3 | 99.6 | 90.1 |
| Hep3B | 65.1 | 89.4 | 94.7 | 92.1 | 90.9 | 94.8 | 94.7 | 93.8 | 90.1 | 97.5 |
| WV-7807 | 75.0 | 95.3 | 101.2 | | 97.3 | 100.9 | 96.4 | 93.4 | 89.2 | 90.6 |
| Hep3B | 75.5 | 95.4 | 95.5 | 88.8 | 103.6 | 92.7 | 99.8 | 103.7 | 93.3 | 87.5 |
| WV-7808 | 72.2 | 98.9 | 117.8 | 109.5 | 116.3 | 110.9 | 109.3 | 116.8 | 111.1 | 104.6 |
| Hep3B | | | | | | | | | | |
| WV-7804 | 4.4 | 3.5 | 25.1 | 45.3 | 68.9 | 74.6 | 73.6 | 82.9 | 81.8 | 86.7 |
| Huh7 | 7.5 | 11.1 | 23.3 | 48.0 | 77.1 | 76.0 | 86.5 | 89.4 | 96.1 | 90.4 |
| WV-7805 | 1.7 | 7.1 | 19.6 | 36.1 | 59.2 | 75.3 | 69.8 | 75.0 | 78.0 | 79.6 |
| Huh7 | 8.4 | 11.9 | 11.6 | 38.4 | 76.2 | 78.4 | 81.1 | 86.9 | 95.8 | 95.0 |
| WV-7806 | 9.8 | 2.8 | 13.8 | 28.8 | 68.7 | 66.6 | 69.7 | 78.6 | 89.4 | 88.6 |
| Huh7 | 3.8 | 7.2 | 12.5 | 39.7 | 68.5 | 82.4 | 80.3 | 79.7 | 87.3 | 98.0 |
| WV-7807 | 3.0 | 12.6 | 25.1 | 46.9 | 78.0 | 85.3 | 98.8 | 92.6 | 85.6 | 85.5 |
| Huh7 | 3.9 | 9.1 | 14.9 | 41.5 | 70.4 | 73.7 | 68.5 | 75.3 | 81.9 | 94.7 |
| WV-7808 | 1.8 | 1.9 | 14.2 | 37.1 | 63.6 | 71.0 | 79.2 | 88.0 | 84.1 | 95.3 |
| Huh7 | 1.4 | 5.2 | 9.1 | 33.2 | 65.2 | 77.4 | 92.3 | 71.2 | 83.5 | 78.1 |

TABLE 107
Activity of oligonucleotides.

| | | |
|---|---|---|
| nM | WV-7805 Hep3B | WV-7805 Huh7 |

| | | | | |
|---|---|---|---|---|
| 20 | 81.8 | 86.1 | 7.1 | 11.9 |
| 8 | 96.9 | 98.5 | 19.6 | 11.6 |
| 3.2 | 90.7 | 98.1 | 36.1 | 38.4 |
| 1.28 | 91.3 | 97.0 | 59.2 | 76.2 |
| 0.512 | 95.5 | 97.9 | 75.3 | 78.4 |
| 0.2048 | 90.5 | 100.7 | 69.8 | 81.1 |
| 0.08192 | 92.2 | 107.6 | 75.0 | 86.9 |
| 0.032768 | 98.5 | 104.0 | 78.0 | 95.8 |
| 0.013107 | 95.0 | 105.8 | 79.6 | 95.0 |

TABLE 108
Activity of oligonucleotides.

| | | | |
|---|---|---|---|
| nM | 2 | 8.25 | 33 |

| | | | | | | |
|---|---|---|---|---|---|---|
| Control | 1.002 | 1.082 | 1.192 | 1.105 | 1.031 | 1.038 |
| WV-1868 | 1.105 | 1.016 | 1.120 | 0.995 | 1.023 | 1.023 |
| WV-3451 | 0.962 | 0.975 | 0.484 | 0.617 | 0.218 | 0.256 |
| WV-3452 | 1.060 | 0.948 | 0.526 | 0.505 | 0.189 | 0.172 |
| WV-3453 | 0.388 | 0.487 | 0.181 | 0.197 | 0.217 | 0.156 |
| WV-3454 | 0.509 | 0.502 | 0.260 | 0.186 | 0.138 | 0.064 |
| WV-3455 | 0.613 | 0.617 | 0.342 | 0.347 | 0.197 | 0.155 |
| WV-3456 | 0.724 | 0.843 | 0.468 | 0.545 | 0.218 | 0.223 |
| WV-3457 | 0.714 | 0.776 | 0.367 | 0.362 | 0.132 | 0.096 |
| WV-3458 | 0.672 | 0.618 | 0.251 | 0.309 | 0.256 | 0.180 |
| WV-3459 | 1.184 | 1.105 | 1.097 | 1.067 | 0.820 | 0.975 |
| WV-3460 | 0.849 | 0.689 | 0.436 | 0.367 | 0.461 | 0.382 |
| WV-3461 | 0.989 | 1.243 | 0.471 | 0.600 | 0.261 | 0.198 |
| WV-3462 | 0.833 | 1.040 | 0.417 | 0.446 | 0.286 | 0.120 |
| Control | 1.002 | 1.082 | 1.192 | 1.105 | 1.031 | 1.038 |
| WV-1868 | 1.105 | 1.016 | 1.120 | 0.995 | 1.023 | 1.023 |
| WV-3437 | 1.045 | 1.067 | 0.564 | 0.501 | 0.256 | 0.181 |
| WV-3438 | 0.861 | 0.989 | 0.935 | 0.760 | 1.009 | 0.982 |
| WV-3439 | 1.009 | 1.016 | 0.770 | 0.734 | 0.576 | 0.530 |
| WV-3440 | | 1.038 | 1.060 | 0.982 | 0.729 | 0.613 |
| WV-3441 | 1.082 | 1.234 | 0.680 | 0.903 | 0.313 | 0.433 |
| WV-3442 | 1.120 | 0.935 | 0.477 | 0.643 | 0.223 | 0.347 |
| WV-3443 | 0.477 | 0.410 | 0.278 | 0.204 | 0.229 | 0.171 |
| WV-3444 | 0.714 | 0.592 | 0.350 | 0.396 | 0.234 | 0.209 |
| WV-3445 | 1.304 | 1.060 | 0.776 | 0.760 | 0.439 | 0.388 |
| WV-3446 | 0.849 | 0.729 | 0.455 | 0.430 | 0.247 | 0.235 |
| WV-3447 | 0.786 | 0.929 | 0.568 | 0.604 | 0.201 | 0.292 |
| WV-3448 | 0.776 | 0.837 | 0.458 | 0.568 | 0.218 | 0.226 |
| WV-3449 | 0.776 | 0.704 | 0.404 | 0.449 | 0.252 | 0.130 |
| WV-3450 | 1.084 | 0.851 | 1.077 | 0.839 | 0.924 | 0.905 |
| Control | 1.002 | 1.082 | 1.192 | 1.105 | 1.031 | 1.038 |
| WV-1868 | 1.105 | 1.016 | 1.120 | 0.995 | 1.023 | 1.023 |
| WV-3423 | 0.634 | 0.600 | 0.407 | 0.419 | 0.315 | 0.280 |
| WV-3424 | 0.634 | 0.754 | 0.350 | 0.445 | 0.124 | 0.286 |
| WV-3425 | 1.002 | 1.023 | 0.729 | 0.643 | 0.347 | 0.345 |
| WV-3426 | 1.023 | 0.797 | 0.487 | 0.427 | 0.364 | 0.367 |
| WV-3427 | 0.849 | 0.897 | 0.709 | 0.781 | 0.621 | 0.572 |
| WV-3428 | 1.052 | 1.089 | 0.831 | 0.922 | 0.942 | 1.002 |
| WV-3429 | 1.060 | 0.982 | 0.588 | 0.604 | 0.401 | 0.340 |
| WV-3430 | 1.074 | 1.184 | 0.935 | 0.942 | 0.505 | 0.449 |
| WV-3431 | 0.600 | 0.675 | 0.391 | 0.364 | 0.208 | 0.237 |
| WV-3432 | 0.975 | 0.955 | 0.630 | 0.639 | 0.276 | 0.326 |
| WV-3433 | 0.596 | 0.685 | 0.240 | 0.270 | 0.215 | 0.146 |
| WV-3434 | 0.885 | 0.714 | 0.261 | 0.342 | 0.206 | 0.193 |
| WV-3435 | 0.584 | 0.584 | 0.350 | 0.311 | 0.288 | 0.261 |
| WV-3436 | 1.074 | 0.797 | 0.760 | 0.661 | 0.515 | 0.477 |
| Control | 0.910 | 1.000 | 0.996 | 1.136 | 1.105 | 1.136 |
| WV-1868 | 1.278 | 1.075 | 0.936 | 1.176 | 0.809 | 0.879 |
| WV-3409 | 0.690 | 0.498 | 0.329 | 0.274 | 0.488 | 0.405 |
| WV-3410 | 0.832 | 0.936 | 0.217 | 0.231 | 0.241 | 0.134 |
| WV-3411 | 0.685 | 0.588 | 0.114 | 0.116 | 0.037 | 0.108 |
| WV-3412 | | 0.873 | 0.383 | 0.286 | 0.089 | 0.070 |
| WV-3413 | 0.855 | 0.838 | 0.336 | 0.263 | 0.030 | 0.071 |
| WV-3414 | 1.105 | 1.024 | 0.798 | 0.709 | 0.290 | 0.333 |
| WV-3415 | 1.024 | | 0.298 | 0.185 | 0.484 | 0.393 |
| WV-3416 | 0.541 | 0.568 | 0.273 | 0.260 | 0.246 | 0.241 |
| WV-3417 | 0.734 | 0.622 | 0.331 | 0.137 | 0.298 | 0.300 |
| WV-3418 | 0.530 | 0.568 | 0.185 | 0.114 | 0.258 | 0.298 |
| WV-3419 | 0.962 | 0.639 | 0.680 | 0.588 | 0.377 | 0.362 |
| WV-3420 | | 1.113 | 0.956 | 0.771 | 0.375 | 0.159 |
| WV-3421 | 0.502 | 0.443 | 0.169 | 0.148 | 0.218 | 0.228 |
| WV-3422 | 0.923 | 1.083 | 0.680 | 0.516 | 0.365 | 0.372 |
| Control | 0.910 | 1.000 | 0.996 | 1.136 | 1.105 | 1.136 |
| WV-1868 | 1.278 | 1.075 | 0.936 | 1.176 | 0.809 | 0.879 |
| WV-3395 | 0.419 | 0.247 | 0.336 | 0.198 | 0.338 | 0.331 |
| WV-3396 | 1.024 | 0.982 | 0.452 | 0.553 | 0.249 | 0.195 |
| WV-3397 | 0.685 | 0.976 | 0.365 | | 0.182 | 0.096 |
| WV-3398 | 1.053 | | 0.159 | 0.273 | 0.061 | 0.052 |
| WV-3399 | 0.357 | 0.440 | 0.284 | 0.141 | 0.221 | 0.282 |
| WV-3400 | 0.867 | 0.861 | 0.553 | 0.458 | 0.184 | 0.160 |
| WV-3401 | 1.252 | 0.904 | 0.481 | 0.383 | 0.133 | 0.093 |
| WV-3402 | 0.437 | 0.302 | 0.122 | 0.096 | 0.070 | 0.046 |
| WV-3403 | 1.176 | 1.218 | 1.003 | 1.144 | 0.879 | 0.929 |
| WV-3404 | 0.195 | 0.367 | 0.155 | 0.135 | 0.269 | 0.215 |
| WV-3405 | 1.024 | 0.695 | 0.377 | 0.258 | 0.194 | 0.208 |
| WV-3406 | 1.287 | 1.075 | 1.314 | 1.201 | 1.314 | 0.969 |
| WV-3407 | 0.949 | 0.917 | 0.609 | 0.498 | 0.326 | 0.125 |
| WV-3408 | 0.239 | 0.360 | 0.107 | 0.187 | 0.265 | 0.161 |
| Control | 0.910 | 1.000 | 0.996 | 1.136 | 1.105 | 1.136 |
| WV-1868 | 1.278 | 1.075 | 0.936 | 1.176 | 0.809 | 0.879 |
| WV-3381 | 0.560 | 0.391 | 0.103 | 0.108 | 0.146 | 0.199 |
| WV-3382 | 0.949 | 1.024 | 0.393 | 0.271 | 0.132 | 0.211 |
| WV-3383 | 1.031 | 0.996 | 0.617 | 0.458 | 0.455 | 0.133 |
| WV-3384 | 1.252 | 1.060 | 0.402 | 0.416 | 0.136 | 0.133 |
| WV-3385 | 0.962 | 1.098 | 0.407 | 0.410 | 0.221 | 0.123 |
| WV-3386 | 0.680 | 0.440 | 0.186 | 0.246 | 0.265 | 0.176 |
| WV-3387 | 0.269 | 0.191 | 0.100 | 0.067 | 0.081 | 0.141 |
| WV-3388 | 1.168 | 0.982 | 0.849 | 1.053 | 1.168 | 0.892 |
| WV-3389 | 1.083 | 1.031 | 1.399 | 0.879 | 0.771 | 0.804 |
| WV-3390 | 0.676 | 0.580 | 0.226 | 0.265 | 0.035 | 0.051 |
| WV-3391 | 0.505 | 0.396 | 0.187 | 0.153 | 0.107 | 0.176 |
| WV-3392 | 0.462 | 0.362 | 0.139 | 0.116 | 0.093 | 0.070 |
| WV-3393 | 0.273 | 0.391 | 0.102 | 0.111 | 0.060 | 0.044 |
| WV-3394 | 0.509 | 0.405 | 0.263 | 0.133 | 0.109 | 0.097 |

In some tests of PNPLA3 oligonucleotides, APOC3 oligonucleotide WV-1868 (which targets APOC3, a gene different than PNPLA2) is used as a negative control.

TABLE 109A
Activity of oligonucleotides.

| Conc (nM) | 20 | 8 | 3.2 | 1.28 | 0.512 | 0.205 | 0.0819 | 0.0328 | 0.0131 | 0.0052 |
|---|---|---|---|---|---|---|---|---|---|---|
| WV-8148 | 111.7 | 111.5 | 102.6 | 105.8 | 102.9 | 103.1 | 92 | 96.5 | 99.6 | 118.7 |
| Hep3B | 97.3 | 114.3 | 107.8 | 106.6 | 105.8 | 106.6 | 90.9 | 100.8 | 95.3 | 105.5 |
| WV-8149 | 128.4 | 118 | 104.2 | 99.8 | 104.8 | 103.2 | 97.1 | 102.1 | 104.6 | 96.5 |
| Hep3B | 135.8 | 131.3 | 108.8 | 108.5 | 94.5 | 106.1 | 107.8 | 99.3 | 103.3 | 102.4 |
| WV-8150 | 120.7 | 123.7 | 115 | 99.8 | 98.6 | 100.9 | 88.5 | 92.3 | 103.1 | 102 |
| Hep3B | 113 | 111.8 | 98.4 | 102.1 | 102.3 | 100 | 103 | 100.8 | 104 | 107.9 |
| WV-8151 | 112.3 | 147 | 108.4 | 103.4 | 104.6 | 104.6 | 111.1 | 100.1 | 102.4 | 100 |
| Hep3B | 119.6 | 124.3 | 106.6 | 101.6 | 110.1 | 104.1 | 113.7 | 100.3 | 100.3 | 104.7 |
| WV-8152 | 119.1 | 139.2 | 113 | 107 | 112.7 | 121 | 112.4 | 111.7 | 88.5 | 114.7 |
| Hep3B | 133.7 | 149 | 118.1 | 103.2 | 101.7 | 101.8 | 102.5 | 94.6 | 98.3 | 104.2 |
| WV-8148 | 26.6 | 41.6 | 64.4 | 90.6 | 91.8 | 86.5 | 98.2 | 90.5 | 99.6 | 94.1 |
| Huh7 | 35.5 | 54 | 65.7 | 87.4 | 85.8 | 87.5 | 86.8 | 100.9 | 99.3 | 90.2 |
| WV-8149 | 23.2 | 33.1 | 64.6 | 83.6 | 87.2 | 94.4 | 82.9 | 94.8 | 79.1 | 92.5 |
| Huh7 | 27.5 | 44.3 | 65.5 | 89.5 | 84.5 | 87.9 | 82.9 | 92 | 94.6 | 85.1 |
| WV-8150 | 26.3 | 26.6 | 55 | 80.4 | 86.7 | 91.5 | 86.9 | 90.1 | 88.4 | 84.7 |
| Huh7 | 20 | 29.6 | 52.7 | 92.1 | 83.4 | 91.9 | 97 | 84 | 86.9 | 93.5 |
| WV-8151 | 13.4 | 28.7 | 59.7 | 82 | 90.6 | 92.4 | 90.2 | 84.4 | 78.1 | 85.7 |
| Huh7 | 23.6 | 32.8 | 63.6 | 77.7 | 101.1 | 92.6 | 91.6 | 100.9 | 83.4 | 90.2 |
| WV-8152 | 13.2 | 36.7 | 58.3 | 90.9 | 94.4 | 95.7 | 76.1 | 84.3 | 90.3 | 85.2 |
| Huh7 | 18.1 | 47.5 | 61 | 84.7 | 93.9 | 86.2 | 96 | 92.6 | 84 | 90.1 |

| nM | 20 | 8 | 3.2 | 1.28 | 0.512 | 0.205 | 0.0819 | 0.0328 | 0.0131 | 0.0052 |
|---|---|---|---|---|---|---|---|---|---|---|
| WV-8194 | 102.9 | 103.8 | 85.5 | 90.8 | 90.8 | 102.3 | 96.8 | 101.1 | 95.8 | 95.5 |
| Hep3B | 93.3 | 95.7 | 87.8 | 90 | 78.5 | 77.4 | 86.4 | 78.1 | 81 | 88.2 |
| WV-8195 | 107.9 | 103.7 | 96.6 | 91.5 | 91.7 | 92.1 | 94.1 | 105.6 | 107.4 | 95.7 |
| Hep3B | 123 | 101.5 | 92.9 | 89.4 | 92 | 86 | 88.3 | 101.9 | 104.3 | 99.3 |
| WV-8196 | 86.5 | 108.1 | 106 | 90.1 | 96.1 | 87.3 | 93.3 | 87.9 | 100.1 | 103.5 |
| Hep3B | 112.8 | 126.8 | 98.2 | 86.9 | 83.2 | 82.4 | 88.8 | 95.5 | 92.8 | 101.9 |
| WV-8197 | 140.8 | 123.5 | 108.9 | 87 | 91.5 | 92.8 | 106.1 | 98.1 | 107.7 | 94.5 |
| Hep3B | 143.8 | 132.1 | 98.1 | 85.6 | 85.3 | 80.7 | 84.6 | 88 | 95.2 | 93.5 |
| WV-8198 | 99.5 | 101 | 89.4 | 85.4 | 88.6 | 94.9 | 88.4 | 95.8 | 95 | 97.2 |
| Hep3B | 119.8 | 90.9 | 85.3 | 95.8 | 93.3 | 80.1 | 82.9 | 82.4 | 86.9 | 88 |
| WV-8194 | 8.1 | 13.3 | 32.2 | 69.5 | 91.6 | 100.4 | 89.2 | 91.8 | 90.4 | 81.5 |
| Huh7 | 7.4 | 31.2 | 38 | 76 | 86.9 | 93.6 | 92.5 | 92 | 87.5 | 104.3 |
| WV-8195 | 7.3 | 27 | 41.1 | 64.1 | 83.2 | 96.5 | 95.1 | 84.7 | 89.8 | 87.3 |
| Huh7 | 14 | 20 | 37.1 | 57.8 | 95 | 94.3 | 85.4 | 90.7 | 97.6 | 85.8 |
| WV-8196 | 8.9 | 19.5 | 26.7 | 64.4 | 78.5 | 88.3 | 83.2 | 88.1 | 81.1 | 81.1 |
| Huh7 | 14.3 | 19.9 | 37.1 | 57.3 | 91.7 | 91.8 | 88.5 | 84.5 | 84 | 91.9 |
| WV-8197 | 4.1 | 27.3 | 40.8 | 65.6 | 88.8 | 91.7 | 95.6 | 96 | 90.6 | 93.3 |
| Huh7 | 14.8 | 26.8 | 44 | 62.4 | 83.9 | 96.5 | 89.1 | 97.9 | 92.9 | 81.8 |
| WV-8198 | 7.9 | 19.9 | 36.5 | 68.6 | 94.6 | 90.6 | 90 | 94 | 90.9 | 89.9 |
| Huh7 | 6.9 | 26.7 | 47.9 | 63.8 | 83.1 | 99.5 | 97.2 | 97.1 | 88.1 | 97.8 |

| Conc (nM) | 20 | 8 | 3.2 | 1.28 | 0.512 | 0.205 | 0.0819 | 0.0328 | 0.0131 | 0.0052 |
|---|---|---|---|---|---|---|---|---|---|---|
| WV-8171 | 102.4 | 98.1 | 108.8 | 104.7 | 111.4 | 107.4 | 102.2 | 112 | 95.9 | 92.4 |
| Hep3B | 94.9 | 94.6 | 89.8 | 102.6 | 97.2 | 101.8 | 96.4 | 98.9 | 97.7 | 90.6 |
| WV-8172 | 105 | 105.3 | 116.3 | 108.9 | 120 | 105.5 | 101.4 | 100.9 | 94.3 | 99.9 |
| Hep3B | 92.8 | 90.5 | 92.7 | 90.1 | 96.8 | 90.6 | 97.9 | 92.5 | 96.7 | 84.7 |
| WV-8173 | 96.9 | 90.2 | 99.1 | 108.6 | 107.1 | 103.6 | 105.9 | 100.6 | 95.6 | 100.2 |
| Hep3B | 104.3 | 96.4 | 99 | 98.2 | 99.9 | 103.3 | 95.7 | 96.4 | 97.7 | 95.1 |
| WV-8174 | 98 | 85.2 | 93.3 | 96.1 | 83.8 | 84.7 | 83 | 86.3 | 93.7 | 105.9 |
| Hep3B | 115.5 | 112.5 | 95.7 | 98.7 | 86.6 | 97.4 | 93.6 | 82.7 | 90.4 | 99.2 |
| WV-8175 | 98.2 | 91 | 90.6 | 88 | 95.9 | 94.2 | 86.7 | 96 | 106.2 | 91.3 |
| Hep3B | 102.6 | 91.1 | 89.4 | 87.1 | 93.7 | 98.8 | 102.7 | 83.9 | 95.7 | 88.1 |
| WV-8171 | 22.1 | 39.4 | 56.7 | 76.8 | 76 | 87.5 | 75.8 | 95.2 | 83.1 | 77.8 |
| Huh7 | 19.2 | 58.8 | 66.5 | 77.9 | 89.6 | 88.9 | 93.8 | 91 | 90.8 | 97.2 |
| WV-8172 | 13.9 | 30 | 54.5 | 80.4 | 88.1 | 87.6 | 79.8 | 85.4 | 82 | 98.4 |
| Huh7 | 20.6 | 43.1 | 54.6 | 78.4 | 89.4 | 92.4 | 93.6 | 99.8 | 109.4 | 105.5 |
| WV-8173 | 37.2 | 44.1 | 78.6 | 106.3 | 113.7 | 106.6 | 105.3 | 103.7 | 97.6 | 93.3 |
| Huh7 | 41 | 75.8 | 85.6 | 100.6 | 111.3 | 103.2 | 114.4 | 117.5 | 107.3 | 107.7 |
| WV-8174 | 30.2 | 47.6 | 66.3 | 69.3 | 92.1 | 85.6 | 85.6 | 76.1 | 87.3 | 89.8 |
| Huh7 | 19 | 40.9 | 52.4 | 77.2 | 92.2 | 96.6 | 88.8 | 92 | 92.1 | 91.8 |
| WV-8175 | 11.5 | 26.5 | 45.9 | 69.1 | 86.1 | 85.6 | 82.6 | 92.9 | 97 | 83.7 |
| Huh7 | 12.5 | 26.7 | 45.2 | 77.6 | 86.4 | 94.5 | 102.9 | 91.7 | 92 | 85.9 |
| nM | 20 | 8 | 3.2 | 1.28 | 0.512 | 0.205 | 0.0819 | 0.0328 | 0.0131 | 0.0052 |
| WV-8217 | 88.4 | 85.5 | 87.5 | 86.9 | 89.2 | 90 | 98.6 | 98.5 | 97.1 | 86.5 |
| Hep3B | 93.3 | 83.5 | 76.7 | 80.1 | 78 | 77.9 | 77.6 | 74.8 | 79.8 | 86.6 |
| WV-8218 | 100.6 | 94.9 | 92 | 93.1 | 99.6 | 96 | 98.2 | 95.5 | 92.9 | 87.4 |
| Hep3B | 99.3 | 96.4 | 99.2 | 96.3 | 95.5 | 100.8 | 100.5 | 93.6 | 96.7 | 96.3 |
| WV-8219 | 147.3 | 128.9 | 107.6 | 106.1 | 104.8 | 106.8 | 98.6 | 101.5 | 96.5 | 110.7 |
| Hep3B | 126.8 | 103.2 | 94.8 | 93.5 | 94.2 | 96 | 100.6 | 103.3 | 96.7 | 102.9 |
| WV-8220 | 104.2 | 115.4 | 103.9 | 97 | 106.9 | 102.2 | 99.4 | 105.1 | 99.1 | 95.3 |
| Hep3B | 100 | 89.8 | 96.8 | 95.5 | 107.4 | 106.4 | 104.3 | 95.9 | 117 | 101 |
| WV- | 110.2 | 104.7 | 97.7 | 102.6 | 104.1 | 106.4 | 102.6 | 100.3 | 102 | 98.4 |
| 8221Hep3B | 94.5 | 98.5 | 101.6 | 99.7 | 99.4 | 108.4 | 103 | 107.1 | 100.3 | 103.3 |
| WV-8217 | 26.8 | 37.9 | 58.2 | 81.9 | 94 | 101.3 | 112 | 105.2 | 97.8 | 28.2 |
| Huh7 | 27.7 | 41 | 57.6 | 90.7 | 109.2 | 102.4 | 110.5 | 109.9 | 105.5 | 105.2 |
| WV-8218 | 21.6 | 45.8 | 56.1 | 77.7 | 86.9 | 97.3 | 82.6 | 88.5 | 86.1 | 73.9 |
| Huh7 | 20.9 | 28.8 | 55.3 | 78.1 | 106.9 | 107.3 | 107.6 | 95.6 | 104.1 | 82.8 |
| WV-8219 | 31.5 | 31 | 42.7 | 62.7 | 92 | 91.7 | 85.7 | 75.7 | 77.2 | 85.7 |
| Huh7 | 26 | 31.8 | 47.9 | 75 | 85.5 | 92.1 | 83.6 | 89.7 | 93.1 | 91.3 |
| WV-8220 | 4.8 | 16.3 | 31.8 | 66.1 | 78.6 | 77.8 | 79.7 | 71.7 | 81.1 | 80.3 |
| Huh7 | 11.8 | 30.1 | 54.4 | 77.4 | 105.6 | 98.6 | 90.5 | 85.7 | 93.9 | 85.5 |
| WV-8221 | 6.2 | 21.4 | 31.8 | 48.6 | 78.3 | 85.8 | 75.5 | 83.8 | 85.3 | 93 |
| Huh7 | 4.6 | 19.1 | 35.2 | 59.6 | 104.8 | 105.8 | 99.2 | 100.1 | 93.2 | 88.7 |

TABLE 109B
Activity of oligonucleotides.
IC50 in Huh7 cells (mutant allele):

| Oligonucleotide | IC50 (nM) | Oligonucleotide | IC50 (nM) |
|---|---|---|---|
| WV-8148 | 7.3 | WV-8197 | 3.2 |
| WV-8149 | 9.2 | WV-8198 | 3.5 |
| WV-8150 | 5.5 | WV-8217 | 5.4 |
| WV-8151 | 7.9 | WV-8218 | 5.9 |
| WV-8152 | 12.6 | WV-8219 | 3.1 |
| WV-8171 | 11.2 | WV-8220 | 5.5 |
| WV-8172 | 5.2 | WV-8221 | 2.6 |
| WV-8173 | 12 | WV-8194 | 3.5 |
| WV-8174 | 6.6 | WV-8195 | 3 |
| WV-8175 | 4.2 | WV-8196 | 2.8 |

TABLE 110
Activity of oligonucleotides.
Huh7 cells:

| | 50 | 20 | 8 | 3.2 | 1.28 | 0.512 | 0.204 | 0.081 | 0.032 | 0.013 |
|---|---|---|---|---|---|---|---|---|---|---|
| WV-3861 | 23.1 | 43.8 | 72.8 | 101.4 | 102.4 | 103.3 | 85.5 | 91.9 | 93.7 | 92.0 |
| | 30.1 | 52.2 | 89.3 | 103.4 | 95.9 | 93.4 | 99.4 | 104.5 | 89.7 | 104.8 |
| WV-7805 | 8.0 | 13.3 | 32.7 | 65.4 | 87.0 | 83.1 | 91.2 | 84.1 | 76.1 | 85.3 |
| | 10.1 | 22.7 | 49.1 | 82.1 | 87.8 | 81.8 | 82.5 | 77.8 | 87.9 | 79.1 |
| WV-7828 | 5.3 | 15.3 | 26.8 | 60.5 | 88.1 | 85.0 | 92.1 | 85.8 | 84.2 | 90.3 |
| | 4.3 | 10.0 | 41.1 | 54.4 | 79.6 | 90.2 | 89.0 | 90.2 | 99.3 | 83.6 |
| WV-7851 | 4.1 | 4.4 | 20.0 | 42.9 | 63.8 | 85.3 | 77.7 | 79.3 | 84.6 | 93.4 |
| | 6.7 | 6.6 | 20.3 | 47.5 | 86.7 | 79.7 | 97.4 | 97.4 | 85.0 | 89.4 |
| WV-8149 | 18.4 | 29.4 | 35.5 | 71.2 | 88.8 | 91.2 | 75.3 | 84.9 | 86.5 | 90.6 |
| | 46.2 | 21.9 | 47.2 | 80.7 | 86.5 | 93.9 | 83.9 | 90.5 | 101.4 | 95.3 |
| WV-8172 | 20.5 | 13.7 | 30.4 | 53.8 | 73.3 | 85.2 | 74.8 | 80.7 | 87.0 | 84.4 |
| | 20.3 | 21.9 | 41.3 | 59.0 | 78.7 | 83.5 | 91.1 | 92.5 | 91.8 | 104.3 |
| WV-8195 | 23.2 | 11.4 | 15.4 | 64.8 | 71.9 | 74.1 | 82.8 | 87.0 | 88.5 | 76.4 |
| | 22.7 | 14.6 | 23.5 | 62.1 | 80.2 | 85.0 | 99.0 | 99.6 | 104.0 | 100.4 |
| WV-8218 | 6.6 | 13.1 | 13.4 | 53.7 | 73.0 | 93.6 | 94.6 | 93.8 | 88.0 | 92.3 |
| | 20.2 | 14.0 | 30.6 | 57.7 | 97.4 | 117.6 | 99.8 | 101.2 | 114.2 | 109.0 |
| WV-3864 | 3.7 | 22.4 | 60.6 | 94.6 | 92.3 | 99.2 | 88.2 | 97.2 | 92.0 | 104.1 |
| | 18.9 | 27.5 | 68.5 | 114.8 | 95.1 | 113.1 | 100.1 | 117.6 | 110.2 | 118.9 |
| WV-7808 | 6.6 | 12.6 | 34.8 | 66.1 | 73.0 | 88.8 | 93.9 | 90.6 | 91.2 | 94.2 |
| | 6.4 | 14.2 | 32.3 | 80.2 | 106.0 | 106.7 | 107.6 | 89.2 | 103.4 | 97.6 |
| WV-7831 | 4.8 | 7.9 | 27.1 | 62.1 | 80.6 | 82.5 | 91.4 | 92.8 | 93.6 | 93.7 |
| | 8.0 | 13.1 | 29.4 | 62.2 | 90.7 | 122.5 | 105.5 | 120.7 | 115.5 | 97.9 |
| WV-7854 | 2.0 | 7.1 | 21.5 | 49.2 | 74.1 | 83.3 | 116.8 | 75.8 | 83.4 | 95.0 |
| | 5.0 | 5.3 | 17.0 | 50.4 | 100.7 | 99.7 | 103.2 | 99.8 | 101.7 | 90.1 |
| WV-8152 | | 14.4 | 33.7 | 80.7 | 82.8 | 80.8 | 84.3 | 86.5 | 89.7 | 88.2 |
| | | 23.4 | 40.1 | 77.3 | 106.3 | 96.3 | 97.7 | 99.9 | 97.1 | 79.5 |
| WV-8175 | 17.6 | 13.5 | 18.2 | 48.5 | 69.7 | 87.0 | 72.8 | 91.4 | 86.5 | 81.6 |
| | 22.4 | 16.5 | 19.2 | 51.1 | 108.5 | 89.7 | 87.4 | 102.0 | 91.2 | 95.4 |
| WV-8198 | 16.6 | 8.3 | 15.1 | 43.7 | 78.0 | 81.9 | 82.7 | 75.3 | 91.5 | 88.2 |
| | 7.1 | 5.1 | 23.9 | 46.3 | 93.6 | 103.2 | 103.6 | 93.8 | 125.9 | 96.0 |
| WV-8221 | 16.0 | 9.6 | 12.4 | 31.8 | 73.1 | 101.6 | 96.7 | 77.9 | 86.7 | 94.3 |
| | 13.0 | 10.9 | 16.2 | 44.1 | 85.5 | 102.0 | 110.9 | 123.8 | 101.9 | 102.9 |

TABLE 111
Activity of oligonucleotides.

| Oligonucleotide | 2 nM | Oligonucleotide | 2 nM |
|---|---|---|---|
| WV-4098 | 30 | WV-9277 | 44 |
| WV-9273 | 72 | WV-9278 | 55 |
| WV-9274 | 73 | WV-9279 | 39 |
| WV-9275 | 74 | WV-9280 | 82 |
| WV-9276 | 52 | WV-9281 | 68 |

Several PNPLA3 ssRNAi agents were also prepared and tested which have an abasic site, specifically a (phosphaneyl)oxy)propan-1-ol (PS) or 3′-(phosphaneyl)oxy)tetrahydrofuran. Results for oligonucleotide administration at 2 nM is shown, and oligonucleotides were also tested at 0, 0.05, 0.128, 0.32, and 0.8 nM (data not shown). Numbers are approximate and represent residual PNPLA3 mRNA level (PNPLA3/HPRT1), wherein 100 would represent 100% residual mRNA level (0% knockdown) and 0 would represent 0% residual mRNA level (100% knockdown). In the various tables herein, the level of mRNA is measured, unless otherwise noted.

TABLE 112
Activity of oligonucleotides.

| Oligonucleotide | 2 nM | Oligonucleotide | 2 nM |
|---|---|---|---|
| WV-4098 | 31 | WV-4098 | 31 |
| WV-9261 | 62 | WV-9272 | 81 |
| WV-9262 | 69 | WV-9284 | 75 |
| WV-9263 | 72 | | |
| WV-9264 | 62 | | |
| WV-9265 | 56 | | |
| WV-9266 | 64 | | |
| WV-9267 | 43 | | |
| WV-9268 | 69 | | |
| WV-9269 | 71 | | |

Several APOC3 ssRNAi agents were also prepared and tested which have C3 modification. Results for oligonucleotide administration at 2 nM is shown, and oligonucleotides were also tested at 0, 0.05, 0.128, 0.32, and 0.8 nM (data not shown). Numbers are approximate and represent residual PNPLA3 mRNA level (PNPLA3/HPRT1), wherein 100 would represent 100% residual mRNA level (0% knockdown) and 0 would represent 0% residual mRNA level (100% knockdown). In the various tables herein, the level of mRNA is measured, unless otherwise noted.

TABLE 113
Activity of oligonucleotides.

| Oligonucleotide | 25 nM |
|---|---|
| WV-3421 | 13 |
| WV-9434 | 63 |
| WV-9439 | 55 |
| WV-9444 | 37 |
| WV-3421 | 12 |
| WV-9435 | 62 |
| WV-9440 | 37 |
| WV-9445 | 34 |
| WV-3421 | 17 |
| WV-9431 | 92 |
| WV-9436 | 70 |
| WV-9441 | 73 |
| WV-9432 | 53 |
| WV-9437 | 36 |
| WV-9442 | 54 |
| WV-9433 | 77 |
| WV-9438 | 44 |
| WV-9443 | 69 |

Data is shown for 25 nM; oligonucleotides were also tested at 0, 1.6, and 6.2 nM (data not shown). Oligonucleotides were tested in vitro in primary cynomolgus hepatocytes.

TABLE 114
Activity of oligonucleotides.

| | Hep3b (wt) | Huh7 (mutant) |
|---|---|---|
| WV-9890 | 88 | 37 |
| WV-12100 | 103 | 27 |
| WV-9893 | 67 | 10 |
| WV-12101 | 69 | 8 |

Primary cynomolgus hepatocytes. Data is shown for 4 nM. Oligonucleotides were also tested at 0, 0.1, 0.25, 0.66, 1.6, and 10 nM (data not shown). Numbers represent residual PNPLA3 mRNA level (PNPLA3/HPRT1) and numbers are approximate.
WV-9893 and WV-12101 have an asymmetrical format.
Additional oligonucleotides which have an asymmetrical format, but which are stereorandom, were tested, which have the double mutation at P9/P12 (positions 9 and 12). WV-8609, WV-8847, WV-8848, WV-8849 all had an IC50 of around 4 to 5 nM.

TABLE 115A
Activity of oligonucleotides.

| | |
|---|---|
| WV-7805 | 58 |
| WV-8603 | 46 |
| WV-8608 | 73 |
| WV-9889 | 69 |
| WV-9890 | 76 |
| WV-8609 | 26 |
| WV-8601 | 61 |
| WV-8605 | 65 |
| WV-8606 | 105 |
| WV-9891 | 43 |
| WV-9892 | 52 |
| WV-9893 | 115 |

Several PNPLA3 oligonucleotides, some of which have an asymmetrical structure, were tested for stability in rat liver homogenate at 2 days. Numbers represent % of full-length oligonucleotide remaining; numbers are approximate.

TABLE 115B
Activity of oligonucleotides.

| Oligonucleotide | Ligand | mFX1/mHPRT1 |
|---|---|---|
| WV-3969 | Tri-GalNAc | 23 |
| WV-5287 | Tri-PFE ligand | 22 |
| WV-7299 | Bi-GalNAc | 22 |
| WV-7300 | Bi-PFE ligand | 20 |
| WV-7297 | Mono-GalNAc | 74 |
| WV-7298 | Mono-PFE ligand | 43 |

Several oligonucleotides were also prepared which target a mouse homolog of different gene, Factor XI (FXI or F11), and which comprised an additional component, which was a tri-, bi- or mono-antennary ligand which was either a GalNAc or a PFE ligand. These were administered to mice at 0.3, 1 or 3 mpK QDx3. Numbers below represent the mFXI/mHPRT1 mRNA level relative to control at 3 mpk. Mice were also administered oligonucleotides at 0.3 and 1 (nth (data not shown).

TABLE 115C
Oligonucleotides.

| Oligo- | | Naked | Stereo- | SEQ ID |
|---|---|---|---|---|
| nucleotide | Sequence | Sequence | chemistry | NO: |
| WV-7297 | Mod038L001Teo * Geo * Geo * Teo * Aeo * A | TGGTAA | OXXXXXXXXXX | 1953 |
| | * T * m5C * m5C * A * m5C * T * T * T * m5C * | TCCACTT | XXXXXXXXX | |
| | Aeo * Geo * Aeo * Geo * Geo | TCAGAGG | | |
| WV-7298 | Mod039L001Teo * Geo * Geo * Teo * Aeo * A | TGGTAA | OXXXXXXXXXX | 1954 |
| | * T * m5C * m5C * A * m5C * T * T * T * m5C * | TCCACTT | XXXXXXXXX | |
| | Aeo * Geo * Aeo * Geo * Geo | TCAGAGG | | |
| WV-7299 | Mod040L001Teo * Geo * Geo * Teo * Aeo * A | TGGTAA | OXXXXXXXXXX | 1955 |
| | * T * m5C * m5C * A * m5C * T * T * T * m5C * | TCCACTT | XXXXXXXXX | |
| | Aeo * Geo * Aeo * Geo * Geo | TCAGAGG | | |
| WV-7300 | Mod041L001Teo * Geo * Geo * Teo * Aeo * A | TGGTAA | OXXXXXXXXXX | 1956 |
| | * T * m5C * m5C * A * m5C * T * T * T * m5C * | TCCACTT | XXXXXXXXX | |
| | Aeo * Geo * Aeo * Geo * Geo | TCAGAGG | | |
| WV-5287 | Mod034L001Teo * Geo * Geo * Teo * Aeo * A | TGGTAA | OXXXXXXXXXX | 1957 |
| | * T * m5C * m5C * A * m5C * T * T * T * m5C * | TCCACTT | XXXXXXXXX | |
| | Aeo * Geo * Aeo * Geo * Geo | TCAGAGG | | |

The various components (e.g., *, Mod038, etc.) in this table are the same as those in Table 1A. All of these oligonucleotides are single-stranded, though the sequences are split into multiple lines for formatting.
Various APOC3 oligonucleotides were constructed which comprise a tri-, bis- or mono-antennary ligand which is either the PFE ligand or GalNAc. Such oligonucleotides include:

TABLE 115D
Oligonucleotides.

| Oligonucleotide | Ligand | | | |
|---|---|---|---|---|
| WV-6558 | Tri-GaINAc | | | |
| WV-9542 | Tri-PFE | | | |
| WV-9543 | Bis-GaINAc | | | |
| WV-9544 | Bis-PFE | | | |
| WV-9545 | Mono-GaINAc | | | |
| WV-9546 | Mono-PFE | | | |
| WAVE | | | Stereo- | SEQ ID |
| ID | Sequence | Naked Sequence | chemistry | NO: |
| WV- | Mod001L001Aeo * SGeom5CeoTeoTeo * RC | AGCTTCTTGTC | OSOOORSSSRS | 777 |
| 6558 | * ST * ST* SG* RT * SC * SC * RA * SG* | CAGCTTTAT | SRSSROOOS | |
| | SC * RTeoTeoTeoAeo * STeo | | | |
| WV- | Mod083L001Aeo * SGeom5CeoTeoTeo * RC | AGCTTCTTGTC | OSOOORSSSRS | 1634 |
| 9542 | * ST * ST* SG* RT * SC * SC * RA * SG* | CAGCTTTAT | SRSSROOOS | |
| | SC * RTeoTeoTeoAeo * STeo | | | |
| WV- | Mod079L001Aeo * SGeom5CeoTeoTeo * RC | AGCTTCTTGTC | OSOOORSSSRS | 1635 |
| 9543 | * ST * ST* SG* RT * SC * SC * RA * SG* | CAGCTTTAT | SRSSROOOS | |
| | SC * RTeoTeoTeoAeo * STeo | | | |
| WV- | Mod080L001Aeo * SGeom5CeoTeoTeo * RC | AGCTTCTTGTC | OSOOORSSSRS | 1636 |
| 9544 | * ST * ST* SG* RT * SC * SC * RA * SG* | CAGCTTTAT | SRSSROOOS | |
| | SC * RTeoTeoTeoAeo * STeo | | | |
| WV- | Mod081L001Aeo * SGeom5CeoTeoTeo * RC | AGCTTCTTGTC | OSOOORSSSRS | 1637 |
| 9545 | * ST * ST* SG* RT * SC * SC * RA * SG* | CAGCTTTAT | SRSSROOOS | |
| | SC * RTeoTeoTeoAeo * STeo | | | |
| WV- | Mod082L001Aeo * SGeom5CeoTeoTeo * RC | AGCTTCTTGTC | OSOOORSSSRS | 1638 |
| 9546 | * ST * ST* SG* RT * SC * SC * RA * SG* | CAGCTTTAT | SRSSROOOS | |
| | SC * RTeoTeoTeoAeo * STeo | | | |

The various components (e.g., *, Mod083, etc.) in this table are the same as those in Table 1A. All of these oligonucleotides are single-stranded, though the sequences are split into multiple lines for formatting.

TABLE 115E
Activity of oligonucleotides

| Day | 0 | 8 | 15 | 22 | 29 | 36 | 43 | 50 |
|---|---|---|---|---|---|---|---|---|
| PBS | 1.52 | 0.95 | 1.50 | 0.56 | 0.96 | 1.07 | 1.57 | 1.74 |
| | 0.59 | 0.73 | 0.74 | 0.87 | 0.90 | 0.90 | 0.73 | 0.71 |
| | 1.21 | 0.99 | 1.10 | 1.34 | 0.89 | 0.82 | 0.62 | 0.78 |
| | 0.67 | 1.14 | 0.89 | 0.99 | 0.89 | 0.86 | 0.95 | 0.86 |
| | 1.01 | 1.20 | 0.76 | 1.24 | 1.35 | 1.36 | 1.13 | 0.91 |
| WV-8877 | 1.56 | 1.24 | 1.67 | 1.59 | 2.37 | 1.56 | 1.47 | 2.27 |
| | 0.78 | 0.73 | 0.85 | 0.80 | 1.15 | 0.61 | 0.75 | 1.19 |
| | 1.08 | 0.81 | 1.42 | | 1.84 | 1.21 | 1.73 | 3.05 |
| | 0.71 | 1.21 | 0.74 | 0.62 | 1.02 | 1.07 | 0.95 | 1.48 |
| | 1.28 | 1.21 | 0.60 | 0.80 | 1.13 | 1.50 | 0.86 | |
| WV-6558 | 2.74 | 0.06 | 0.05 | 0.06 | 0.11 | 0.38 | 0.69 | 1.43 |
| | 1.15 | 0.17 | 0.05 | 0.04 | 0.09 | 0.27 | 0.01 | 0.81 |
| | 0.38 | 0.04 | 0.05 | 0.10 | 0.18 | 0.45 | 0.53 | 1.07 |
| | 0.44 | | | | | | | |
| | 0.41 | 0.04 | 0.04 | 0.08 | 0.09 | 0.11 | 0.13 | 0.22 |
| WV-9542 | 1.10 | 0.23 | 0.05 | 0.07 | 0.13 | 0.23 | 0.32 | 0.78 |
| | 0.71 | 0.03 | 0.02 | 0.04 | 0.06 | 0.09 | 0.20 | 0.28 |
| | 0.59 | 0.05 | 0.04 | 0.08 | 0.16 | 0.72 | 0.90 | 0.80 |
| | 0.32 | 0.03 | 0.02 | 0.04 | 0.09 | 0.37 | 0.54 | 0.55 |
| | 0.40 | 0.03 | 0.03 | 0.06 | 0.21 | 0.39 | 0.49 | 0.58 |
| WV-9543 | 0.48 | 0.03 | 0.05 | 0.09 | 0.08 | 0.21 | 0.27 | 0.49 |
| | 1.19 | 0.06 | 0.06 | 0.09 | 0.06 | 0.09 | 0.57 | 0.96 |
| | 0.79 | 0.05 | 0.04 | 0.17 | 0.06 | 0.15 | 0.42 | 0.80 |
| | 0.79 | 0.09 | 0.03 | 0.28 | 0.20 | 0.17 | 0.28 | 0.59 |
| | 0.48 | 0.04 | 0.02 | 0.08 | 0.06 | 0.12 | 0.17 | 0.32 |
| WV-9544 | 0.91 | | 0.04 | 0.06 | 0.06 | 0.19 | 0.26 | 0.67 |
| | 0.94 | 0.10 | 0.03 | 0.08 | 0.09 | 0.15 | 0.34 | 0.76 |
| | 1.72 | 0.19 | 0.04 | 0.07 | 0.09 | 0.25 | 0.60 | 0.83 |
| | 1.92 | 0.28 | 0.07 | 0.10 | 0.11 | 0.13 | 0.26 | 0.56 |
| | 0.81 | 0.04 | 0.05 | 0.11 | 0.12 | 0.20 | 0.32 | 0.73 |
| WV-9545 | 0.49 | 0.03 | 0.07 | 0.16 | 0.21 | 0.32 | 0.66 | 0.60 |
| | 1.14 | 0.22 | 0.04 | 0.10 | 0.15 | 0.58 | 0.76 | 0.97 |
| | 0.58 | 0.03 | 0.04 | 0.15 | 0.27 | 0.67 | 1.16 | 0.97 |
| | 0.64 | 0.03 | 0.04 | 0.19 | 0.42 | 0.98 | 1.38 | 0.96 |
| | 0.60 | 0.05 | 0.03 | 0.08 | | | | |
| WV-9546 | 3.33 | 0.20 | 0.06 | 0.27 | 0.24 | 0.49 | 1.13 | 1.31 |
| | 1.03 | 0.11 | 0.04 | 0.09 | 0.14 | 0.46 | 0.55 | 0.68 |
| | 1.20 | 0.28 | 0.12 | 0.20 | 0.31 | 0.95 | 1.75 | 1.39 |
| | 0.71 | 0.15 | 0.04 | 0.19 | 0.39 | 0.26 | 0.75 | 0.36 |
| | 0.18 | 0.04 | 0.02 | 0.20 | 0.28 | 0.21 | 0.56 | 0.56 |

All oligonucleotides were administered to animals at a 3 mg/kg single dose (s.c.) at day 1. In addition, WV-6558 and WV-9542 were also administered to animals at a 1 mg/kg single dose (s.c.) at day 1. Serum was collected at days 0, 8, 15, 22, 29, 36, 43, and 50. Each group contained 5 animals. PBS and WV-8877 (which targets a gene which is not APOC3) were negative controls.
Numbers indicate relative APOC3 protein level, wherein 1.00 represents 100% relative to PBS.
In various in vivo studies, including this one, tested animals were transgenic mice expressing the human APOC3 gene.

TABLE 115F
Part I. Oligonucleotide accumulation in the liver

| PBS | WV-6558 | WV-9542 | WV-9543 | WV-9544 | WV-9545 | WV-9546 |
|---|---|---|---|---|---|---|
| 0 | 2.95 | 1.73 | 3.52 | 3.82 | 2.02 | 4.27 |
| 0 | 2.46 | 1.69 | 2.49 | 4.19 | 1.99 | 1.37 |
| 0 | 2.48 | 0.45 | 1.14 | 2.74 | 1.30 | 1.29 |
| 0 | 1.85 | 1.09 | 2.12 | 2.26 | 1.14 | 1.25 |
| 0 | 1.79 | 1.43 | 4.26 | 1.88 | 1.07 | 0.82 |

Oligonucleotide accumulation in the liver was also analyzed after a single 3 mg/kg dose, 30 min. Numbers indicate pg of oligonucleotide/g of tissue. Tested animals were transgenic mice expressing the human APOC3 gene.
In the same experiment: Oligonucleotide accumulation in the liver was also analyzed for WV-6558 and WV-9542 after a single 1 mg/kg dose, 30 min. Numbers indicate pg of oligonucleotide/g of tissue.

| | WV-6558 | WV-9542 |
|---|---|---|
| PBS | 1 mpk | 1 mpk |
| 0 | 1.92 | 0.46 |
| 0 | 1.77 | 1.08 |
| 0 | 1.43 | 0.56 |
| 0 | 0.68 | 0.30 |
| 0 | 0.18 | 0.67 |

TABLE 115F
Part II. Oligonucleotide accumulation in the liver

| PBS | WV-6558 | WV-9542 | WV-9543 | WV-9544 | WV-9545 | WV-9546 |
|---|---|---|---|---|---|---|
| 0 | 3.30 | 2.93 | 6.83 | 4.56 | 3.55 | 3.83 |
| 0 | 3.49 | 2.20 | 6.56 | 4.45 | 2.23 | 4.05 |
| 0 | 3.18 | 1.34 | 4.58 | 2.72 | 1.94 | 2.28 |
| 0 | 2.41 | 1.61 | 3.87 | 2.31 | 3.03 | 2.12 |
| 0 | 1.43 | 2.90 | 4.10 | 2.36 | 1.85 | 3.50 |

Oligonucleotide accumulation in the liver was also analyzed after a single 3 mg/kg dose, 8 days. Numbers indicate ug of oligonucleotide/g of tissue. Tested animals were transgenic mice expressing the human APOC3 gene.
In the same experiment: Oligonucleotide accumulation in the liver was also analyzed for WV-6558 and WV-9542 after a single 1 mg/kg (1 mpk) dose, 8 days. Numbers indicate μg of oligonucleotide/g of tissue.

| | WV-6558 | WV-9542 |
|---|---|---|
| PBS | 1 mpk | 1 mpk |
| 0 | 0.72 | 1.08 |
| 0 | 0.74 | 1.20 |
| 0 | 0.60 | 0.75 |
| 0 | 0.55 | 0.57 |
| 0 | 0.63 | 0.63 |

The data show the efficacy of various ligands conjugated to APOC3 oligonucleotides; these same ligands can also be conjugated onto PNPLA3 oligonucleotides.
Table 116. Activity of oligonucleotides.
Various PNPLA3 RNAi agents were tested for stability in rat liver homogenate. Numbers represent percent of full-length oligonucleotide remaining at 5 days; oligonucleotides were also tested at 2 days (data not shown); and numbers are approximate. Some oligonucleotides comprise a 5′-DNA-T and some oligonucleotides comprise a 5′-Rc-Me-T.

TABLE 117
Activity of oligonucleotides.

| | |
|---|---|
| WV-8095 | 62 |
| WV-9495 | 61 |
| WV-9499 | 86 |
| WV-8701 | 49 |
| WV-9496 | 71 |
| WV-9500 | 99 |

Various PNPLA3 oligonucleotides were also tested for efficacy with an additional component which is a tri-antennary GalNAc conjugate. Oligonucleotides were tested in vitro on Huh7-148 OE cells (which comprise the mutant allele of PNPLA3) at 10 nM. Numbers represent PNPLA3 mRNA levels (PNPLA3/HPRT1), and numbers are approximate. In many cases, the oligonucleotides did not demonstrate significant knockdown of wild-type PNPLA3 in cynomolgus (non-human primate or NHP) hepatocytes. For example, WV-8132, WV-8600, WV-9868 and WV-9860 did not demonstrate significant knockdown of wild-type PNPLA3 in cynomolgus (non-human primate or NHP) hepatocytes when tested at up to 10 nM (data not shown).

TABLE 118
Activity of oligonucleotides.

| Negative control | 100 | Negative control | 100 |
|---|---|---|---|
| WV-993 | 117 | WV-993 | 117 |
| WV-7805 | 20 | WV-8600 | 47 |
| WV-8132 | 54 | WV-8564 | 47 |
| WV-8566 | 67 | WV-8596 | 62 |
| WV-8599 | 82 | WV-8597 | 38 |
| WV-9859 | 56 | | |
| WV-9670 | 57 | | |
| WV-993 | 117 | WV-993 | 117 |
| WV-9868 | 48 | WV-9860 | 65 |
| WV-9869 | 50 | WV-9861 | 58 |
| WV-9870 | 53 | WV-9862 | 62 |

Various PNPLA3 oligonucleotides were tested in vitro in cells after treatment with oligonucleotide. In this table, 100.00 would represent 100% PNPLA3 mRNA level and 0.00 would represent 0% PNPLA3 mRNA after treatment with oligonucleotides.

TABLE 119
Activity of oligonucleotides.

| Time (mins) | 0 | 5 | 10 | 15 | 20 | 30 | 45 | 60 |
|---|---|---|---|---|---|---|---|---|
| WV-7805 + WV-8807 | 100.0 | 94.1 | 93.4 | 88.6 | 90.6 | 82.8 | 74.5 | 73.4 |
| WV-8603 + WV-8807 | 100.0 | 93.1 | 89.7 | 84.4 | 91.0 | 82.4 | 73.0 | 66.4 |
| WV-8608 + WV-8807 | 100.0 | 95.4 | 92.2 | 89.8 | 87.4 | 81.4 | 79.7 | 72.1 |
| WV-9889 + WV-8807 | 100.0 | 90.9 | 87.7 | 81.9 | 85.7 | 74.4 | 72.9 | 66.4 |
| WV-9890 + WV-8807 | 100.0 | 92.7 | 89.6 | 85.4 | 88.7 | 77.0 | 75.8 | 66.8 |
| WV-7805 + WV-8808 | 100.0 | 99.5 | 97.7 | 98.1 | 96.9 | 96.2 | 95.6 | 93.4 |
| WV-8603 + WV-8808 | 100.0 | 102.2 | 99.4 | 100.3 | 99.1 | 98.5 | 99.2 | 95.6 |
| WV-8608 + WV-8808 | 100.0 | 98.8 | 97.5 | 96.9 | 95.9 | 96.9 | 95.5 | 94.1 |
| WV-9889 + WV-8808 | 100.0 | 99.9 | 99.2 | 99.5 | 98.6 | 97.8 | 97.2 | 96.3 |
| WV-9890 + WV-8808 | 100.0 | 107.5 | 100.7 | 100.8 | 99.1 | 104.2 | 98.3 | 97.5 |
| WV-8601 + WV-8807 | 100.0 | 93.1 | 90.9 | 90.4 | 91.4 | 88.2 | 85.6 | 80.1 |
| WV-8605 + WV-8807 | 100.0 | 98.3 | 96.0 | 96.4 | 96.0 | 96.0 | 87.1 | 86.7 |
| WV-8606 + WV-8807 | 100.0 | 90.1 | 91.6 | 90.7 | 90.9 | 86.6 | 82.4 | 79.1 |
| WV-8609 + WV-8807 | 100.0 | 92.1 | 89.0 | 83.8 | 85.5 | 75.6 | 75.7 | 69.0 |
| WV-8601 + WV-8808 | 100.0 | 99.0 | 100.2 | 100.2 | 97.8 | 97.6 | 97.2 | 94.1 |
| WV-8605 + WV-8808 | 100.0 | 100.7 | 99.7 | 100.9 | 98.4 | 99.1 | 98.5 | 94.6 |
| WV-8606 + WV-8808 | 100.0 | 101.2 | 97.6 | 98.1 | 96.3 | 97.0 | 96.5 | 93.9 |
| WV-8609 + WV-8808 | 100.0 | 96.7 | 93.7 | 98.6 | 96.8 | 95.6 | 96.2 | 94.5 |
| WV-9891 + WV-8807 | 100.0 | 91.6 | 88.3 | 86.1 | 87.9 | 79.8 | 75.6 | 75.2 |
| WV-9892 + WV-8807 | 100.0 | 93.2 | 86.9 | 83.5 | 84.3 | 74.2 | 64.2 | 58.6 |
| WV-9893 + WV-8807 | 100.0 | 94.6 | 88.6 | 86.6 | 88.6 | 77.4 | 69.0 | 65.6 |
| WV-9891 + WV-8808 | 100.0 | 98.3 | 98.6 | 96.9 | 95.0 | 94.2 | 92.8 | 89.8 |
| WV-9892 + WV-8808 | 100.0 | 100.7 | 101.8 | 100.7 | 99.3 | 97.9 | 97.4 | 95.7 |
| WV-9893 + WV-8808 | 100.0 | 100.1 | 100.3 | 100.2 | 99.3 | 96.3 | 96.1 | 93.5 |
| WV-9894 + WV-8807 | 100.0 | 96.2 | 90.1 | 85.1 | 84.7 | 79.5 | 76.8 | 74.9 |
| WV-9895 + WV-8807 | 100.0 | 97.0 | 92.5 | 87.1 | 84.3 | 77.0 | 71.8 | 70.7 |
| WV-9896 + WV-8807 | 100.0 | 98.2 | 93.2 | 86.0 | 81.8 | 74.8 | 69.2 | 70.0 |
| WV-9894 + WV-8808 | 100.0 | 98.8 | 97.1 | 97.4 | 96.1 | 94.0 | 95.4 | 91.4 |
| WV-9895 + WV-8808 | 100.0 | 99.9 | 97.1 | 98.5 | 99.3 | 96.1 | 96.4 | 93.6 |
| WV-9896 + WV-8808 | 100.0 | 99.2 | 99.0 | 98.3 | 96.4 | 95.6 | 93.8 | 90.6 |

Various PNPLA3 oligonucleotides were tested in vitro in an RNaseH assay.
PNPLA3 oligonucleotides were incubated in the presence of target RNA which was the wt allele (WV-8808) or the 148 allele (WV-8807). Numbers represent the percentage of target RNA (WV-8808 or WV-8807) remaining. In this table, 100.00 would represent 100% PNPLA3 mRNA level and 0.00 would represent 0% PNPLA3 mRNA after treatment with oligonucleotides.
The PNPLA3 oligonucleotides WV-980, WV-9893, WV-8606 and WV-7805 also significantly reduced PNPLA3 148 mutant mRNA levels in Huh7 cells with PNLA3 148 mutation (to between about 25 to 55% residual mutant PNPLA3, relative to HPRT1, at 12.5 nM), but these oligonucleotides did not significantly reduce wt PNPLA3 levels in Huh7 cells with wt PNPLA3 (about 90% or more residual wt PNPLA3 level at 12.5 nM).

TABLE 120
Activity of oligonucleotides.

| | Conc. (nM) (exp10) | | | | | | | |
|---|---|---|---|---|---|---|---|---|
| | 1.398 | 0.796 | 0.194 | −0.408 | −1.010 | −1.612 | −2.214 | −2.816 |
| WV-3380 | 0.059 | 0.193 | 0.568 | 0.809 | 0.809 | 0.917 | 1.032 | 0.983 |
| | 0.092 | 0.365 | 0.720 | 0.862 | | 1.004 | 1.121 | |
| WV-3986 | 0.379 | 0.444 | 0.673 | 0.790 | 0.870 | 0.870 | 0.993 | 0.933 |
| | 0.486 | 0.551 | 0.752 | 0.901 | 1.007 | 0.939 | 0.876 | 1.140 |
| WV-3987 | 0.400 | 0.521 | 0.742 | 0.870 | 0.959 | 0.889 | 0.986 | 0.952 |
| | 0.451 | 0.594 | 0.914 | 0.966 | 1.086 | 0.972 | 1.021 | 1.072 |
| WV-3988 | 0.496 | 0.521 | 0.742 | 1.021 | 0.946 | 1.079 | 0.907 | 0.959 |
| | 0.328 | 0.615 | 0.920 | 1.057 | 1.064 | 0.979 | 0.901 | 1.133 |
| WV-3393 | 0.115 | 0.165 | 0.438 | 0.795 | 0.882 | 1.028 | 1.086 | 0.986 |
| | 0.080 | 0.218 | 0.555 | 0.835 | 0.952 | 1.064 | 0.933 | 1.057 |
| WV-3989 | 0.316 | 0.279 | 0.547 | 0.790 | 0.852 | 0.993 | 0.966 | 1.000 |
| | 0.295 | 0.412 | 0.651 | 0.889 | 1.049 | 1.140 | 0.986 | 1.173 |
| WV-3990 | 0.259 | 0.444 | 0.624 | 0.979 | 1.109 | 1.021 | 1.007 | 0.993 |
| | 0.274 | 0.559 | 0.779 | 0.959 | 1.079 | 1.042 | 1.049 | 1.164 |

Various PNPLA3 oligonucleotides were tested in vitro in Hep3B cells at 48 hours after treatment with oligonucleotide. In this table, 1.00 would represent 100% PNPLA3 mRNA level and 0.00 would represent 0% PNPLA3 mRNA after treatment with oligonucleotides.

TABLE 121
Activity of oligonucleotides.

| | Conc. (nM) (exp10) | | | | | | | |
|---|---|---|---|---|---|---|---|---|
| | 1.398 | 0.796 | 0.194 | −0.408 | −1.010 | −1.612 | −2.214 | −2.816 |
| WV-3402 | 0.146 | 0.207 | 0.457 | 0.907 | 0.933 | 0.959 | 0.939 | 0.952 |
| | 0.104 | 0.319 | 0.697 | 0.914 | 0.926 | 1.028 | 1.094 | 1.102 |
| WV-3991 | 0.216 | 0.423 | 0.582 | 0.858 | 0.907 | 0.966 | 0.870 | 0.966 |
| | 0.303 | 0.500 | 0.722 | 1.049 | 0.895 | 0.979 | 1.042 | 1.035 |
| WV-3992 | 0.303 | 0.384 | 0.594 | 0.823 | 0.818 | 0.907 | 0.847 | 0.852 |
| | 0.321 | 0.423 | 0.673 | 0.914 | 0.933 | 0.907 | 0.959 | 1.057 |

Various PNPLA3 oligonucleotides were tested in vitro in Hep3B cells at 48 hours after treatment with oligonucleotide. In this table, 1.00 would represent 100% PNPLA3 mRNA level and 0.00 would represent 0% PNPLA3 mRNA after treatment with oligonucleotides.

TABLE 122
Activity of oligonucleotides.

| | Conc. (nM) (exp 10) | | | | | | | |
|---|---|---|---|---|---|---|---|---|
| | 1.398 | 0.796 | 0.194 | −0.408 | −1.010 | −1.612 | −2.214 | −2.816 |
| WV-3387 | 0.091 | 0.205 | 0.527 | 0.811 | 1.070 | 0.881 | 0.977 | 0.971 |
| | 0.081 | 0.135 | 0.391 | 0.851 | 1.033 | 0.964 | 0.944 | 1.070 |
| WV-3993 | 0.998 | 0.912 | 1.026 | 1.062 | 1.308 | 1.019 | 1.019 | 0.957 |
| | 0.869 | 0.912 | 1.123 | 1.077 | 1.084 | 1.048 | 1.055 | 1.100 |
| WV-3994 | 0.944 | 0.991 | 1.195 | 1.040 | 1.107 | 1.012 | 1.123 | 0.971 |
| | 0.857 | 0.991 | 1.146 | 1.077 | 1.092 | 1.138 | 1.033 | 1.154 |

Various PNPLA3 oligonucleotides were tested in vitro in Hep3B cells at 48 hours after treatment with oligonucleotide. In this table, 1.00 would represent 100% PNPLA3 mRNA level and 0.00 would represent 0% PNPLA3 mRNA after treatment with oligonucleotides.

TABLE 123
Activity of oligonucleotides.

| | Conc. (nM) (exp10) | | | | | | | |
|---|---|---|---|---|---|---|---|---|
| | 1.398 | 0.796 | 0.194 | −0.408 | −1.010 | −1.612 | −2.214 | −2.816 |
| WV-3391 | 0.176 | 0.264 | 0.502 | 0.912 | 0.944 | 1.170 | 1.077 | 0.887 |
| | 0.141 | 0.230 | 0.531 | 0.788 | 1.040 | 1.146 | 1.005 | 1.005 |
| WV-3995 | 0.925 | 1.026 | 0.977 | 1.131 | 1.162 | 0.984 | 1.123 | 0.811 |
| | 0.751 | 0.957 | | 1.123 | | 1.162 | 1.062 | 0.971 |
| WV-3996 | 0.893 | 0.899 | 0.833 | 0.991 | 1.203 | 1.146 | 1.138 | 0.964 |
| | 0.875 | 0.899 | 0.851 | 1.187 | | 1.123 | 1.040 | 1.131 |

Various PNPLA3 oligonucleotides were tested in vitro in Hep3B cells at 48 hours after treatment with oligonucleotide. In this table, 1.00 would represent 100% PNPLA3 mRNA level and 0.00 would represent 0% PNPLA3 mRNA after treatment with oligonucleotides.

TABLE 124
Activity of oligonucleotides.

| | | |
|---|---|---|
| 0.312 nM | 1.25 nM | 5 nM |

| | | | | | | |
|---|---|---|---|---|---|---|
| Control | 76.5 | 84.9 | 111.7 | 106.6 | 113.9 | 99.1 |
| WV-3380 | 86.7 | 79.0 | 58.4 | 60.3 | 27.2 | 28.6 |
| wv-4054 | 60.3 | 49.1 | 67.6 | 53.4 | 65.5 | 45.5 |
| wv-4098 | 62.0 | 43.3 | 57.7 | 50.9 | 38.5 | 52.8 |
| WV-6585 | 58.8 | 56.9 | 71.3 | 90.8 | 89.4 | 79.0 |
| WV-6586 | 82.1 | 53.7 | 82.5 | 79.5 | 89.0 | 66.2 |
| WV-6587 | 49.9 | 45.3 | 103.1 | 56.0 | 74.7 | 77.9 |
| WV-6588 | 58.6 | 60.9 | 82.1 | 84.9 | 86.5 | 85.3 |
| WV-6589 | 61.8 | 51.3 | 92.2 | 94.4 | 77.8 | 83.5 |
| WV-6590 | 63.7 | 64.3 | 62.9 | 83.9 | 85.5 | 60.8 |
| WV-6591 | 83.3 | 71.4 | 74.7 | 75.2 | 76.3 | 94.2 |
| WV-6592 | 49.7 | 39.8 | 51.4 | 40.4 | 54.3 | 39.4 |
| WV-6593 | 68.1 | 77.7 | 58.3 | 89.3 | 64.6 | 70.7 |
| WV-6594 | 82.1 | 53.7 | 58.7 | 59.1 | 61.6 | 62.2 |
| WV-4054 | 58.7 | 35.6 | 55.3 | 49.8 | 66.0 | 60.3 |
| WV-6595 | 40.9 | 52.4 | 58.0 | 54.5 | 60.5 | 56.2 |
| WV-6596 | 48.6 | 40.2 | 57.2 | 49.4 | 46.9 | 49.2 |
| WV-6597 | 27.7 | 31.4 | 41.8 | 52.7 | 61.3 | 45.0 |
| WV-6598 | 40.1 | 35.4 | 59.1 | 53.6 | 44.5 | 42.3 |
| WV-6599 | 37.3 | 54.3 | 73.0 | 61.8 | 76.6 | 69.6 |
| WV-6600 | 64.7 | 67.5 | 88.7 | 105.6 | 95.3 | 115.7 |
| WV-6601 | 74.2 | 48.0 | 64.4 | 51.4 | 97.0 | 81.9 |
| WV-6602 | 64.7 | 51.6 | 64.0 | 63.3 | 95.8 | 64.1 |
| WV-6603 | 57.8 | 40.4 | 85.7 | 73.9 | 67.1 | 71.1 |
| WV-6604 | 50.7 | 50.5 | 57.8 | 47.0 | 72.0 | 47.1 |
| WV-6605 | 52.1 | 52.5 | 58.2 | 57.8 | 58.9 | 57.8 |
| WV-6606 | 27.1 | 56.6 | 52.4 | 51.1 | 77.7 | 53.9 |
| WV-6607 | 35.7 | 41.6 | 44.0 | 37.0 | 76.2 | 53.7 |

Various PNPLA3 oligonucleotides were tested in vitro in cells after treatment with oligonucleotide. In this table, 100.00 would represent 100% PNPLA3 mRNA level and 0.00 would represent 0% PNPLA3 mRNA after treatment with oligonucleotides.

TABLE 125
Activity of oligonucleotides.

| | | |
|---|---|---|
| 0.312 nM | 1.25 nM | 5 nM |

| | | | | | | |
|---|---|---|---|---|---|---|
| Control | 76.5 | 84.9 | 111.7 | 106.6 | 113.9 | 99.1 |
| WV-3380 | 86.7 | 79.0 | 58.4 | 60.3 | 27.2 | 28.6 |
| wv-4054 | 60.3 | 49.1 | 67.6 | 53.4 | 65.5 | 45.5 |
| wv-4098 | 62.0 | 43.3 | 57.7 | 50.9 | 38.5 | 52.8 |
| WV-6608 | 74.0 | 71.3 | 64.3 | 80.2 | 90.8 | 81.4 |
| WV-6609 | 88.6 | 51.9 | 71.6 | 57.7 | 66.3 | 61.2 |
| WV-6610 | 51.1 | 59.5 | 65.1 | 51.7 | 61.2 | 60.6 |
| WV-6611 | 40.9 | 47.5 | 61.7 | 57.3 | 63.2 | 75.1 |
| WV-6612 | 50.9 | 59.8 | 50.6 | 53.3 | 75.4 | 54.3 |
| WV-6613 | 39.0 | 49.1 | 49.5 | 35.6 | 53.8 | 43.5 |
| WV-6614 | 51.6 | 65.2 | 43.6 | 59.6 | 47.8 | 67.1 |
| WV-6615 | 45.7 | 70.0 | 40.0 | 41.6 | 44.1 | 53.2 |
| WV-5305 | 61.4 | 82.3 | 73.1 | 100.3 | 83.3 | 101.0 |
| WV-6616 | 63.6 | 49.3 | 67.0 | 74.3 | 62.0 | 70.1 |
| WV-6617 | 67.5 | 45.2 | 44.0 | 54.6 | 54.9 | 59.2 |
| WV-6618 | 53.4 | 44.2 | 45.4 | 46.4 | 66.5 | 32.9 |
| WV-6619 | 56.7 | 28.9 | 64.4 | 50.3 | 49.7 | 42.9 |
| WV-6620 | 61.8 | 55.6 | 57.8 | 90.1 | 37.8 | 52.5 |
| WV-6621 | 63.3 | 51.1 | 55.0 | 73.3 | 31.4 | 54.7 |
| WV-6622 | 67.5 | 34.7 | 55.2 | 48.0 | 27.4 | 61.7 |
| WV-6623 | 57.1 | 56.5 | 73.3 | 88.7 | 78.4 | 95.9 |

Various PNPLA3 oligonucleotides were tested in vitro in cells after treatment with oligonucleotide. In this table, 100.00 would represent 100% PNPLA3 mRNA level and 0.00 would represent 0% PNPLA3 mRNA after treatment with oligonucleotides.

TABLE 126
Activity of oligonucleotides.

| | | |
|---|---|---|
| 0.312 nM | 1.25 nM | 5 nM |

| | | | | | | |
|---|---|---|---|---|---|---|
| Control | 76.5 | 84.9 | 111.7 | 106.6 | 113.9 | 99.1 |
| WV-3380 | 86.7 | 79.0 | 58.4 | 60.3 | 27.2 | 28.6 |
| wv-4054 | 60.3 | 49.1 | 67.6 | 53.4 | 65.5 | 45.5 |
| wv-4098 | 62.0 | 43.3 | 57.7 | 50.9 | 38.5 | 52.8 |
| WV-6624 | 59.2 | 71.5 | 52.2 | 78.3 | 64.7 | 59.0 |
| WV-6625 | 53.7 | 50.7 | 49.4 | 41.9 | 53.1 | 51.1 |
| WV-6626 | 62.3 | 58.2 | 65.0 | 70.4 | 39.7 | 53.9 |
| WV-6627 | 57.5 | 51.1 | 66.9 | 59.1 | 49.2 | 52.9 |
| WV-6628 | 44.8 | 48.6 | 61.5 | 59.8 | 50.4 | 63.3 |
| WV-6629 | 61.4 | 54.4 | 59.5 | 86.7 | 52.5 | 58.3 |
| WV-6630 | 40.8 | 54.1 | 44.5 | 46.3 | 56.3 | 54.3 |
| WV-6631 | 61.0 | 61.4 | 47.6 | 111.2 | 75.1 | 70.1 |
| WV-6632 | 67.5 | 96.3 | 93.1 | 79.0 | 84.8 | 86.5 |
| WV-6633 | 61.1 | 56.4 | 51.8 | 37.1 | 40.6 | 46.0 |
| WV-6634 | 66.7 | 65.7 | 52.8 | 51.9 | 39.1 | 39.0 |
| WV-6635 | 90.3 | 63.6 | 72.6 | 68.6 | 66.7 | 70.6 |
| WV-6636 | 68.0 | 40.3 | 57.7 | 55.9 | 45.1 | 50.6 |
| WV-6637 | 68.0 | 46.2 | 46.9 | 60.4 | 40.2 | 69.4 |
| WV-6638 | 46.2 | 38.0 | 64.8 | 41.3 | 40.3 | 32.9 |

Various PNPLA3 oligonucleotides were tested in vitro in cells after treatment with oligonucleotide. In this table, 100.00 would represent 100% PNPLA3 mRNA level and 0.00 would represent 0% PNPLA3 mRNA after treatment with oligonucleotides.

TABLE 127
Activity of oligonucleotides.

| | | |
|---|---|---|
| 0.312 nM | 1.25 nM | 5 nM |

| | | | | | | |
|---|---|---|---|---|---|---|
| Control | 76.5 | 84.9 | 111.7 | 106.6 | 113.9 | 99.1 |
| WV-3380 | 86.7 | 79.0 | 58.4 | 60.3 | 27.2 | 28.6 |
| wv-4054 | 60.3 | 49.1 | 67.6 | 53.4 | 65.5 | 45.5 |
| wv-4098 | 62.0 | 43.3 | 57.7 | 50.9 | 38.5 | 52.8 |
| WV-6639 | 94.8 | 81.0 | 113.1 | 90.2 | 68.5 | 69.4 |
| WV-6640 | 91.3 | 78.0 | 60.4 | 87.5 | 87.7 | 61.7 |
| WV-6641 | 76.4 | 113.6 | 83.1 | 87.6 | 59.6 | 65.0 |
| WV-6642 | 95.0 | 104.3 | 90.6 | 98.5 | 74.8 | 73.5 |
| WV-6643 | 126.6 | 90.1 | 96.8 | 77.1 | 60.0 | 75.3 |
| WV-6644 | 125.8 | 94.5 | 89.9 | 85.1 | 81.4 | 63.5 |
| WV-6645 | 93.1 | 74.3 | 97.7 | 66.4 | 68.9 | 40.8 |
| WV-6646 | 83.5 | 80.4 | 85.1 | 60.9 | 56.7 | 33.7 |
| WV-6647 | 92.9 | 77.8 | 91.8 | 79.8 | 125.9 | 62.3 |
| WV-6648 | 104.4 | 88.7 | 92.0 | 111.5 | 67.3 | 73.3 |
| WV-6649 | 106.9 | 85.8 | 79.7 | 85.5 | 78.4 | 74.5 |
| WV-6650 | 94.6 | 79.2 | 87.4 | 91.5 | 66.5 | 97.9 |
| WV-6651 | 116.4 | 74.8 | 92.2 | 96.8 | 58.0 | 57.3 |
| WV-6652 | 114.1 | 70.2 | 110.9 | 94.0 | 88.6 | 66.4 |
| WV-6653 | 116.1 | 89.1 | 90.0 | 100.0 | 77.3 | 72.9 |
| WV-6654 | 84.9 | 99.0 | 101.1 | 128.1 | 67.4 | 70.9 |
| WV-6655 | 102.0 | 99.5 | 116.9 | 83.8 | 114.7 | 85.6 |
| WV-6656 | 115.3 | 119.9 | 114.7 | 85.2 | 101.0 | 108.4 |
| WV-6657 | 88.6 | 94.1 | 114.1 | 109.7 | 94.6 | 100.4 |
| WV-6658 | 114.4 | 92.2 | 131.2 | 134.7 | 133.3 | 90.6 |
| WV-6659 | 116.9 | 104.2 | 122.1 | 96.6 | 99.8 | 122.3 |
| WV-6660 | 104.7 | 79.5 | 124.1 | 100.2 | 79.7 | 88.5 |

Various PNPLA3 oligonucleotides were tested in vitro in cells after treatment with oligonucleotide. In this table, 100.00 would represent 100% PNPLA3 mRNA level and 0.00 would represent 0% PNPLA3 mRNA after treatment with oligonucleotides.

TABLE 129
Activity of oligonucleotides.

| | 0 | 0.1 nM | 0.4 nM | 3.0 nM | 12.5 nM |
|---|---|---|---|---|---|
| WV-4098 | 96.1 | 76.8 | 61.7 | 58.2 | 53.6 |
| | 105.7 | 73.2 | 58.3 | 47.9 | 57.9 |
| WV-7776 | 107.4 | 92.5 | 117.0 | 85.0 | 74.7 |
| | 85.4 | 93.1 | 102.7 | 73.4 | 62.5 |
| WV-7777 | 107.4 | 107.4 | 88.2 | 63.2 | 71.7 |
| | 90.9 | 90.9 | 73.4 | 73.1 | 59.8 |

Various PNPLA3 oligonucleotides were tested in vitro in cells after treatment with oligonucleotide. In this table, 100.00 would represent 100% PNPLA3 mRNA level and 0.00 would represent 0% PNPLA3 mRNA after treatment with oligonucleotides.

TABLE 130
Activity of oligonucleotides.

| | WV-4098 | WV-7465 | WV-8076 |
|---|---|---|---|
| 0 | 103.4 | 112.5 | 101.0 |
| | 86.2 | 95.5 | 109.7 |
| 0.02 nM | 82.5 | 62.8 | 67.2 |
| | 91.2 | 61.6 | 79.1 |
| 0.1 nM | 54.4 | 39.5 | 46.5 |
| | 56.3 | 48.4 | 72.2 |
| 0.4 nM | 49.6 | 31.2 | 46.6 |
| | 48.1 | 42.7 | 43.9 |
| 3.125 nM | 21.9 | 39.2 | 82.0 |
| | 33.7 | 37.1 | 79.7 |

Various PNPLA3 oligonucleotides were tested in vitro in Huh7 cells. Residual levels of PNPLA3 mRNA are shown, wherein PNPLA3 is relative to HPRT1. In this table, 100.00 would represent 100% PNPLA3 mRNA level and 0.00 would represent 0% PNPLA3 mRNA after treatment with oligonucleotides.

TABLE 131
Activity of oligonucleotides.

| | WV-4098 | WV-8080 | WV-8081 |
|---|---|---|---|
| 0 | 103.4 | 83.3 | 79.2 |
| | 86.2 | 109.8 | 89.9 |
| 0.02 nM | 82.5 | 58.1 | 97.2 |
| | 91.2 | 89.1 | 92.4 |
| 0.1 nM | 54.4 | 71.2 | 91.7 |
| | 56.3 | 72.1 | 95.5 |
| 0.4 nM | 49.6 | 79.8 | 94.4 |
| | 48.1 | 97.9 | 108.2 |
| 3.125 nM | 21.9 | 59.3 | 115.6 |
| | 33.7 | 62.0 | 122.4 |

Various PNPLA3 oligonucleotides were tested in vitro in Huh7 cells. Residual levels of PNPLA3 mRNA are shown, wherein PNPLA3 is relative to HPRT1. In this table, 100.00 would represent 100% PNPLA3 mRNA level and 0.00 would represent 0% PNPLA3 mRNA after treatment with oligonucleotides.

TABLE 132
Activity of oligonucleotides.

| | WV-4098 | WV-8077 | WV-8078 | WV-8079 |
|---|---|---|---|---|
| 0 | 103.4 | 108.4 | 94.0 | 80.9 |
| | 86.2 | 107.6 | 98.3 | 87.7 |
| 0.02 nM | 82.5 | 102.1 | 96.4 | 71.1 |
| | 91.2 | | 99.8 | 69.3 |
| 0.1 nM | 54.4 | 87.6 | 93.9 | 75.6 |
| | 56.3 | 87.9 | 118.3 | 97.5 |
| 0.4 nM | 49.6 | 83.4 | 91.0 | 88.6 |
| | 48.1 | 97.1 | 116.6 | 120.6 |
| 3.125 nM | 21.9 | 79.8 | 73.7 | 105.4 |
| | 33.7 | 74.8 | 87.7 | |

Various PNPLA3 oligonucleotides were tested in vitro in Huh7 cells. Residual levels of PNPLA3 mRNA are shown, wherein PNPLA3 is relative to HPRT1. In this table, 100.00 would represent 100% PNPLA3 mRNA level and 0.00 would represent 0% PNPLA3 mRNA after treatment with oligonucleotides.

TABLE 133

| | WV-4098 | WV-7465 |
|---|---|---|
| 0 | 103.4 | 112.5 |
| | 86.2 | 95.5 |
| 0.02 nM | 82.5 | 62.8 |
| | 91.2 | 61.6 |
| 0.1 nM | 54.4 | 39.5 |
| | 56.3 | 48.4 |
| 0.4 nM | 49.6 | 31.2 |
| | 48.1 | 42.7 |
| 3.125 nM | 21.9 | 39.2 |
| | 33.7 | 37.1 |

Various PNPLA3 oligonucleotides were tested in Huh7 cells. Residual levels of PNPLA3 mRNA are shown, wherein PNPLA3 is relative to HPRT1. In this table, 100.00 would represent 100% PNPLA3 mRNA level and 0.00 would represent 0% PNPLA3 mRNA after treatment with oligonucleotides.
Several PNPLA3 oligonucleotides were also tested in vitro for cytokine release, including WV-8061, WV-8291, WV-8698, and WV-8700. None of the 4 PNPLA3 ssRNAi agents induced cytokine release (IL-113, IL-6, MCP-1, IL-12p40, IL-12p70, IL-1α, MIP-1 α, MIP-1β, TNFα) in any of the donor samples. In contrast, positive control induced cytokine activation even at low concentrations (0.78 ug/ml).

### Example 27. Example Additional Components of Oligonucleotides

Various oligonucleotides were designed and constructed which comprise various additional components. Various additional PNPLA3 oligonucleotides described herein can also be conjugated to these additional components.

These additional components include those listed herein:

Tri-antennary ligand is also known as Tri-PFE ASPGR ligand or Tri-PFE ligand or Tri-PFE:

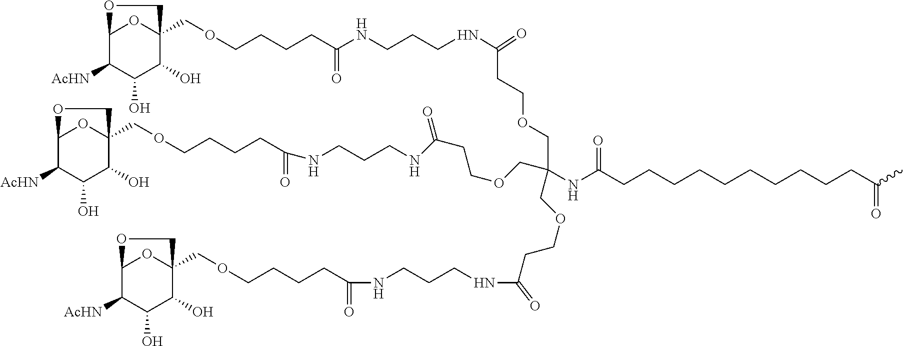

### Bis-Antennary (or Bi-Antennary) Ligand, Also Known as Bis- (or Bi-) Antennary PFE Ligand or Bis- (or Bi-) Antennary PFE ASPGR Ligand or Bis-PFE:

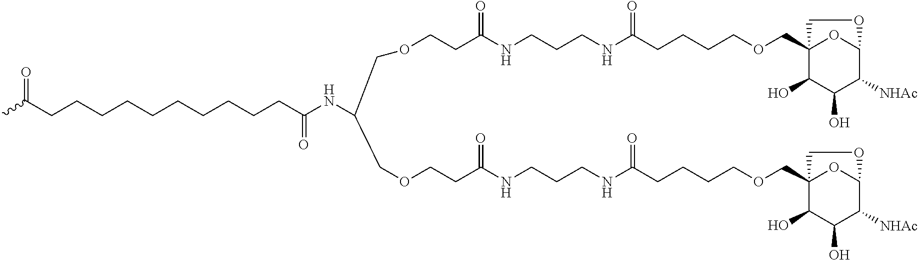

### Mono-Antennary Ligand, Also Known as Mono-Antennary PFE Ligand or Mono-Antennary PFE ASPGR Ligand or Mono-PFE:

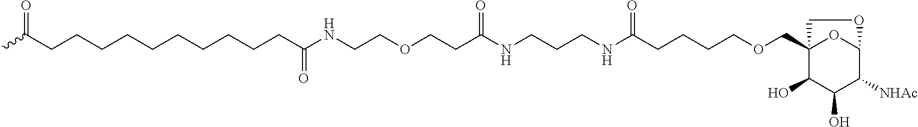

### Tri-Antennary GalNAc or Tri-GalNAc:

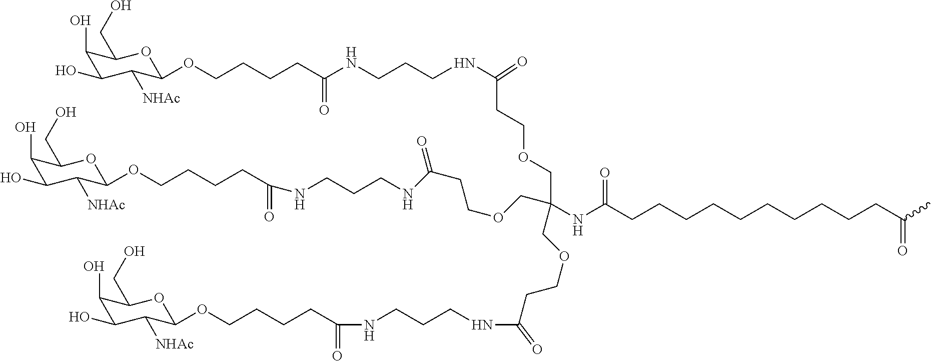

Protected versions of:

### Bis-Antennary (Bi-Antennary) GalNAc or Bis-GalNAc:

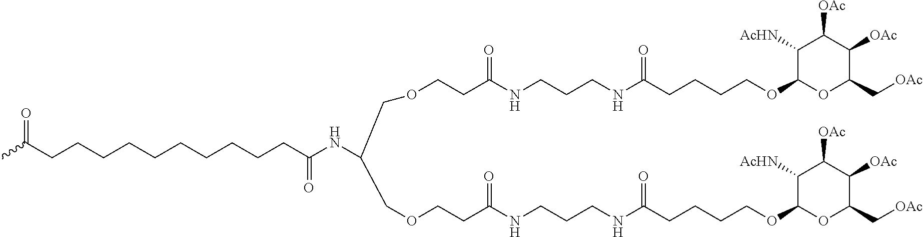

### Mono-Antennary GalNac or Mono-GalNAc:

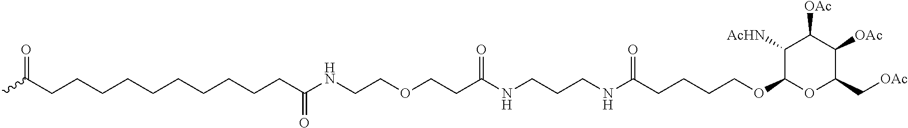

These structures represent the protected versions, as they comprise —OAc (—O-acetate groups). In some embodiments, the Ac groups are removed during de-protection following conjugation of the compound to the oligonucleotide. In some embodiments, de-protection is performed with concentrated ammonium hydroxide, e.g., as described in Example 37B. In the de-protected versions of these structures, —OAc is replaced by —OH.

Some non-limiting examples of processes for production of various additional components are described below:

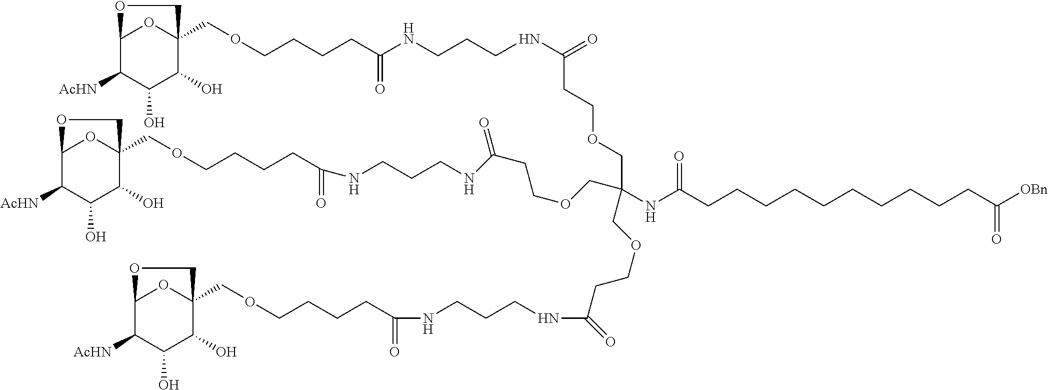 [SMILES: CC(=O)N[C@H]1[C@H]2OC[C@](COCCCCC(=O)NCCCNC(=O)CCOCC(COCCC(=O)NCCCNC(=O)CCCCOC[C@@]34CO[C@@H](O3)[C@H](NC(C)=O)[C@@H](O)[C@H]4O)(COCCC(=O)NCCCNC(=O)CCCCOC[C@@]34CO[C@@H](O3)[C@H](NC(C)=O)[C@@H](O)[C@H]4O)NC(=O)CCCCCCCCCCC(=O)OCc3ccccc3)(O2)[C@H](O)[C@@H]1O]

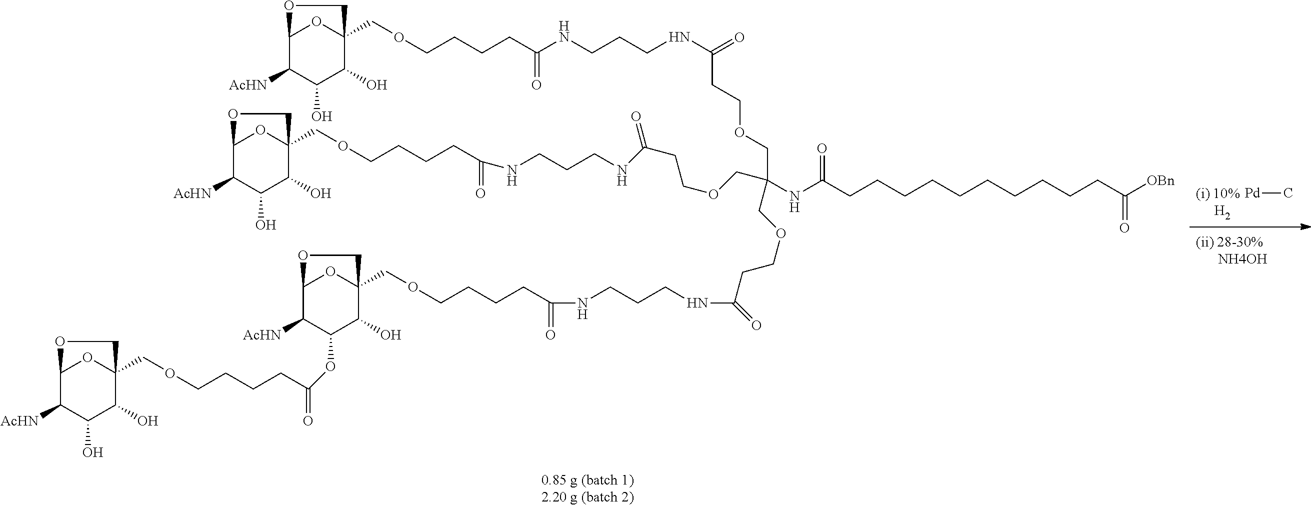

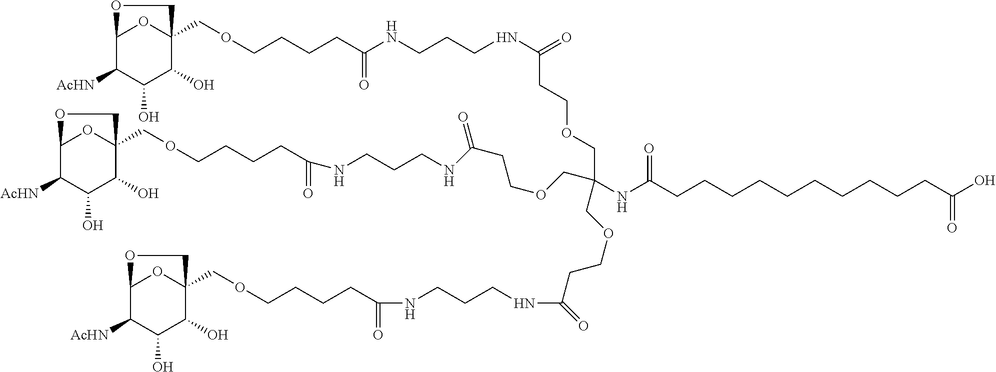 [SMILES: CC(=O)N[C@H]1[C@H]2OC[C@](COCCCCC(=O)NCCCNC(=O)CCOCC(COCCC(=O)NCCCNC(=O)CCCCOC[C@@]34CO[C@@H](O3)[C@H](NC(C)=O)[C@@H](O)[C@H]4O)(COCCC(=O)NCCCNC(=O)CCCCOC[C@@]34CO[C@@H](O3)[C@H](NC(C)=O)[C@@H](O)[C@H]4O)NC(=O)CCCCCCCCCCC(=O)O)(O2)[C@H](O)[C@@H]1O]

purified by C18 cartridge eluting with 0.1% TFA in water and acetonitrile
   0.566 g (GL-N12-55) (containing 4 TFA)
   2.14 g (GL-N12-58) (containing 3.7 TFA)

[chemical structure]

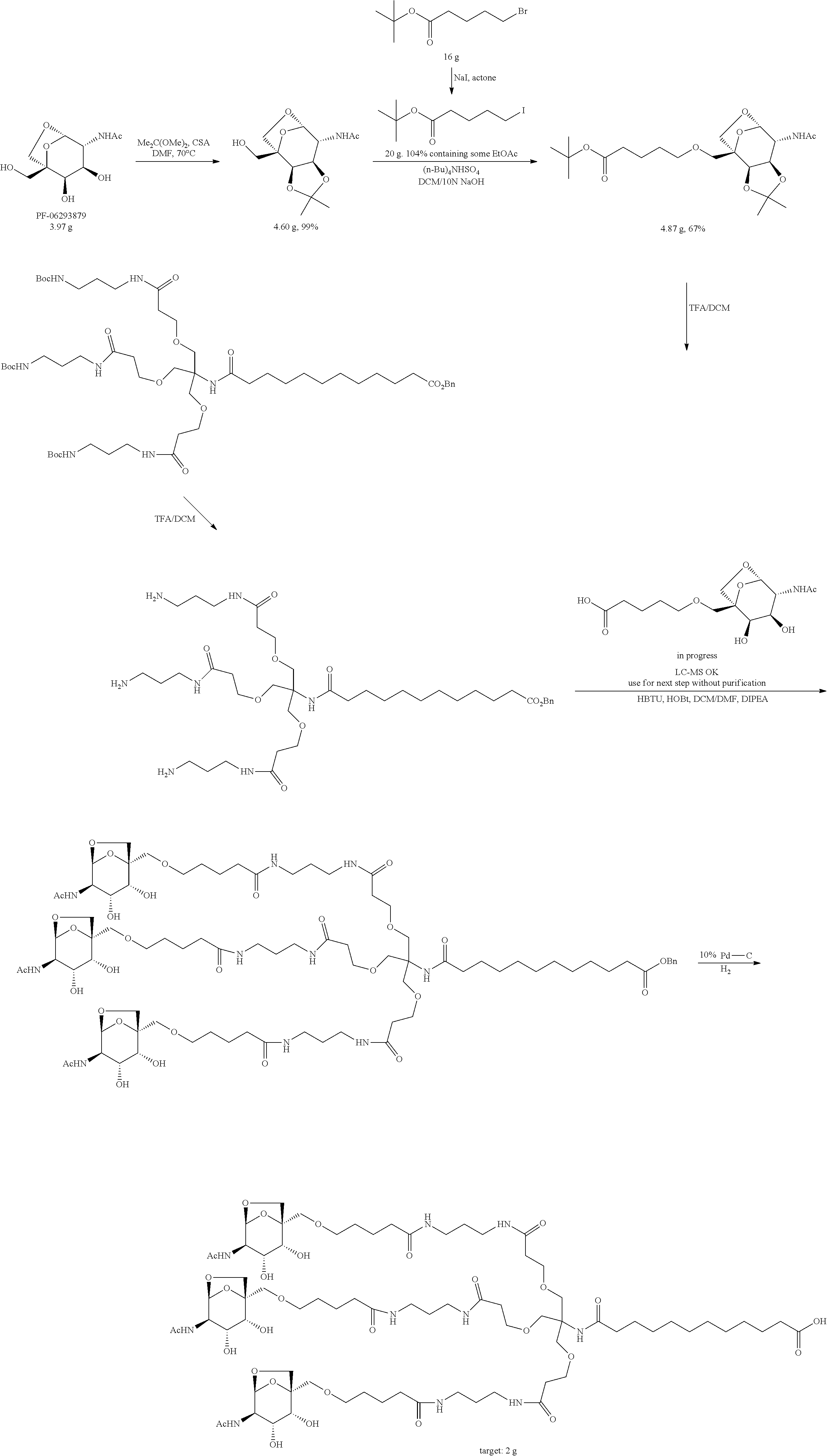

Various additional components described herein can be conjugated to various oligonucleotides described herein.

### Example 28. Example Analytical Methods

1.5 minute run LRMS (low resolution mass spectroscopy): Waters Acqity HSS T3, 2.1 mm×50 mm, C18, 1.7 μm; Mobile Phase: A: 0.1% formic acid in water (v/v); Mobile phase B: 0.1% formic acid in acetonitrile (v/v); Flow-1.25 ml/minute; Initial conditions: A-95%:B-5%; hold at initial from 0.0-0.1 minute; Linear Ramp to A-5%:B-95% over 0.1-1.0 minute; hold at A-5%:B-95% from 1.0-1.1 minute; return to initial conditions 1.1-1.5 minute.

3.0 minute run LRMS (low resolution mass spectroscopy): Waters Acqity HSS T3, 2.1 mm×50 mm, C18, 1.7 μm; Mobile Phase: A: 0.1% formic acid in water (v/v); Mobile phase B: 0.1% formic acid in acetonitrile (v/v); Flow-1.25 ml/minute; Initial conditions: A-95%:B-5%; hold at initial from 0.0-0.1 minute; Linear Ramp to A-5%:B-95% over 0.1-2.6 minute; hold at A-5%:B-95% from 2.6-2.95 minute; return to initial conditions 2.95-3.0 minute.

### 5-carboxypentyl 5,9,16,22-tetraoxo-11,11-bis{[3-oxo-3-({3-[(5-{[3,4,6-tri-O-acetyl-2-(acetylamino)-2-deoxy-β-D-galactopyranosyl]oxy}pentanoyl)amino]propyl}amino)propoxy]methyl}-26-{[3,4,6-tri-O-acetyl-2-(acetylamino)-2-deoxy-β-D-galactopyranosyl]oxy}-13-oxa-4,10,17,21-tetraazahexacos-1-yl phosphate

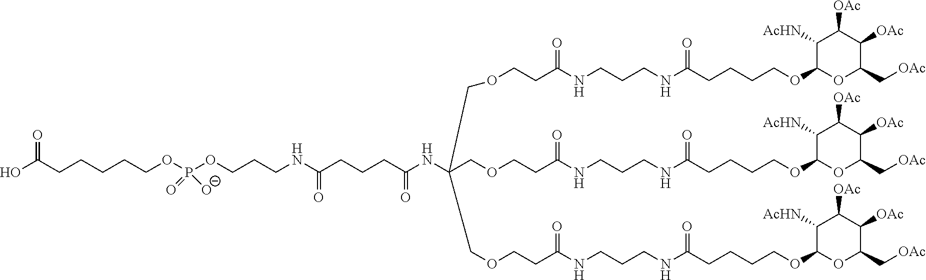 [SMILES: CC(=O)N[C@H]1[C@H](OCCCCC(=O)NCCCNC(=O)CCOCC(COCCC(=O)NCCCNC(=O)CCCCO[C@@H]2O[C@H](COC(C)=O)[C@H](OC(C)=O)[C@H](OC(C)=O)[C@H]2NC(C)=O)(COCCC(=O)NCCCNC(=O)CCCCO[C@@H]2O[C@H](COC(C)=O)[C@H](OC(C)=O)[C@H](OC(C)=O)[C@H]2NC(C)=O)NC(=O)CCCC(=O)NCCCOP(=O)([O-])OCCCCCC(=O)O)O[C@H](COC(C)=O)[C@H](OC(C)=O)[C@@H]1OC(C)=O]

### Reaction Scheme:

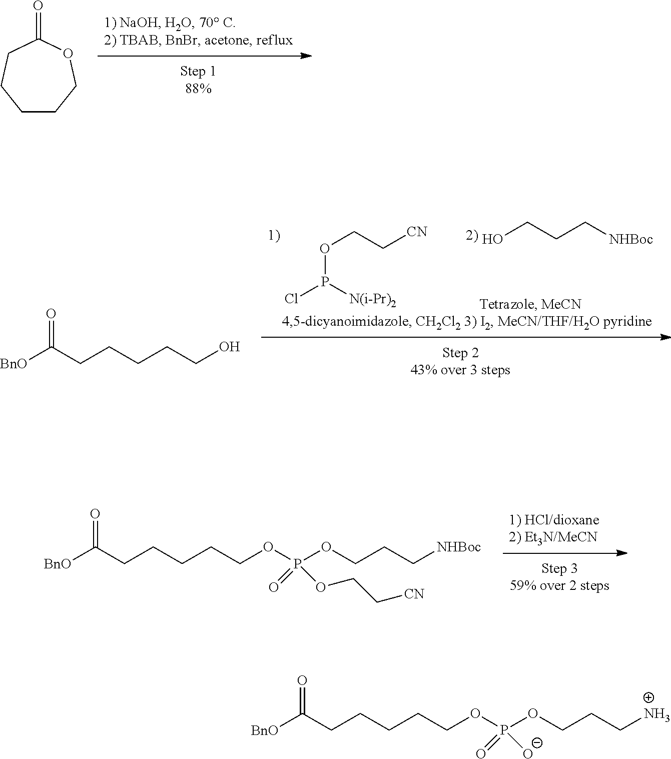

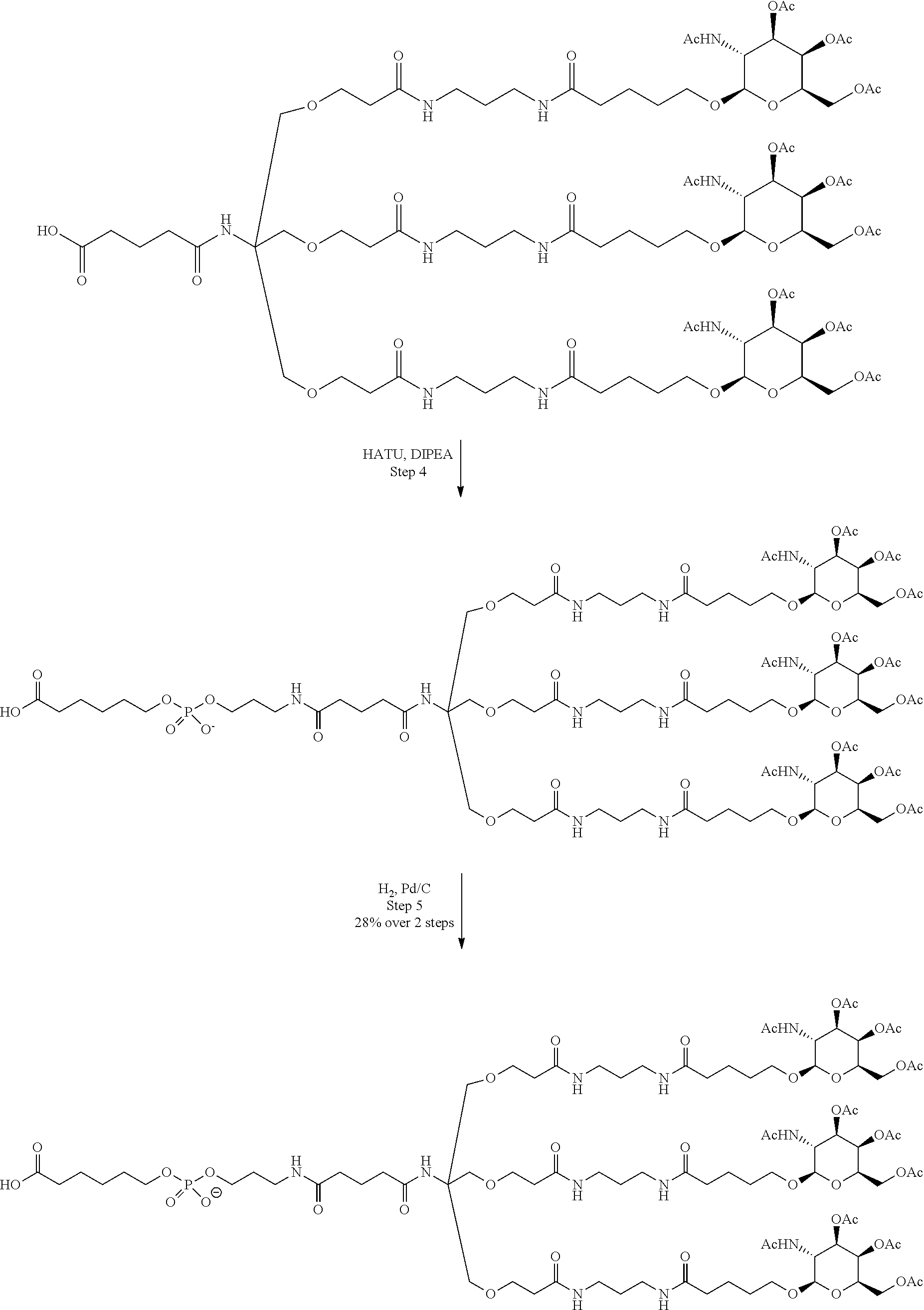

### Step 1: 6-Hydroxyhexanoate

A mixture of sodium hydroxide (9.02 g, 226 mmol) and 6-hexanolactone (25 mL, 0.23 mmol) in water (401 mL) was heated at 70° C. overnight. TLC showed complete consumption of the starting material. The water was removed carefully at 50° C. with a rotary evaporator and the resulting white solid was azeotroped with toluene. After drying under high vacuum overnight, the solid was suspended in acetone (100 mL) and tetrabutylammonium bromide (3.64 g, 11.3 mmol) and benzyl bromide (32.2 mL, 271 mmol) were added. The reaction mixture was heated at reflux until TLC analysis showed complete consumption of intermediate carboxylic acid (96 h). The solvent was removed in vacuo and the residue was partitioned between aqueous hydrochloric acid and ethyl acetate. The aqueous layer was extracted with ethyl acetate (×2). The combined organic extracts were washed with saturated sodium bicarbonate (×2), brine, dried over magnesium sulfate, filtered and concentrated in vacuo. The crude residue was purified by a silica gel plug (20-70% ethyl acetate in heptane) to afford the title compound as a colorless oil (43.9 g, 88%). ¹H NMR (600 MHz, CDCl₃) δ ppm 7.40-7.30 (m, 5H), 5.12 (s, 2H), 3.63 (t, 2H), 2.38 (t, 2H), 1.73-1.65 (m, 2H), 1.62-1.53 (m, 2H), 1.44-1.35 (m, 2H), 1.28 (br.s., 1H).

### Step 2: Benzyl 6-(((3-((tert-butoxycarbonyl)amino)propoxy)(2-cyanoethoxy)phosphoryl)oxy)hexanoate

To a solution of 2-cyanoethyl-N,N-diisopropylchlorophosphoramidite (20.3 g, 67.5 mmol) in anhydrous dichloromethane (150 mL) at 0° C. was slowly added 4,5-dicyanoimidazole (1 M in acetonitrile, 31.5 mL, 31.5 mmol) at 0° C. 6-hydroxyhexanoate (10.0 g, 45.0 mmol) was then added dropwise to the mixture at 0° C. under an inert atmosphere. The mixture was stirred at 0° C. until TLC analysis showed consumption of starting material (1 h). The reaction was quenched with saturated sodium bicarbonate (80 mL). The biphasic mixture was then separated and the aqueous layer was extracted with dichloromethane (2×60 mL). The combined organic phase was washed with brine, dried over anhydrous sodium sulfate, filtered and concentrated to dryness to give the crude product (23.0 g, >100%) as a light yellow oil, which was used in the next step directly. This crude material was dissolved in acetonitrile (50 mL) and added dropwise over 10 min to a solution of 3-(Boc-amino)-1-propanol (10.0 g, 57.2 mmol) and tetrazole (19.1 g, 272 mmol) in anhydrous acetonitrile (300 mL). The resulting colorless solution was stirred at ambient temperature for 1.5 h. TLC showed the starting material was consumed completely. Then a solution of 12 (0.4 M in THF/H₂O/pyridine (78:20:2), 90 mL, 54.4 mmol) was added slowly to the above reaction mixture and at the end of the addition the brown color didn't dissipate. The mixture was stirred at ambient temperature until TLC analysis showed the reaction was complete (1 h). The mixture was quenched with saturated sodium sulfite and concentrated in vacuo to remove the organic solvents. The remaining mixture was diluted with water and extracted with ethyl acetate (×2). The combined organic phase was washed with saturated ammonium chloride and brine, dried over anhydrous sodium sulfate, filtered and concentrated in vacuo. The crude material was purified by silica gel flash chromatography (20-75 ethyl acetate in petroleum ether) to afford the title compound as colorless oil (10.0 g, 43% over three steps). ¹H NMR (400 MHz, CDCl₃) δ ppm 7.41-7.28 (m, 5H), 5.11 (s, 2H), 4.23 (ddd, 2H), 4.18-4.04 (m, 4H), 3.24 (q, 2H), 2.75 (ddd, 2H), 2.38 (t, 2H), 1.87 (dq, 2H), 1.76-1.64 (m, 4H), 1.43 (s, 9H), 1.31-1.20 (m, 2H).

### Step 3: 3-Ammoniopropyl (6-(benzyloxy)-6-oxohexyl)phosphate

To a solution of benzyl 6-(((3-((tert-butoxycarbonyl)amino)propoxy)(2-cyanoethoxy)phosphoryl)oxy)hexanoate (9.00 g, 17.6 mmol) in anhydrous 1,4-dioxane (36 mL) was added hydrochloric acid (100 mL, 400 mmol, 4 M in dioxane) dropwise at 0° C. The resulting colorless solution was stirred at ambient temperature for 1.5 h. The mixture was concentrated to dryness to give the crude product (7.90 g) as a colorless gum, which was used in the next step directly. To a solution of this crude material acetonitrile (72 mL) was added triethylamine (36 mL, 0.26 mmol). The resulting white suspension was stirred at 25° C. for 16 h. The mixture was then concentrated and the crude material was purified by silica gel flash chromatography (5-50% methanol in dichloromethane, 1% ammonium hydroxide) to afford the title compound as a white solid (3.70 g, 59% over two steps). ¹H NMR (400 MHz, CD₃OD) δ ppm 7.41-7.27 (m, 5H), 5.11 (s, 2H), 3.95 (dt, 2H), 3.85 (q, 2H), 3.08 (t, 2H), 2.39 (t, 2H), 1.94 (dq, 2H), 1.78-1.56 (m, 4H), 1.51-1.34 (m, 2H). LCMS (m/z) for C₁₆H₂₇NO₆P⁺ (M+H)⁺ 360.1; retention time=0.677 min (UPLC 1.5 min method).

### Step 4: 6-(Benzyloxy)-6-oxohexyl 26-{[4,6-di-O-acetyl-2-(acetylamino)-2-deoxy-β-D-galactopyranosyl]oxy}-5,9,16,22-tetraoxo-11,11-bis {[3-oxo-3-({3-[(5-{[3,4,6-tri-O-acetyl-2-(acetylamino)-2-deoxy-β-D-galactopyranosyl]oxy}pentanoyl)amino]propyl}amino)propoxy]methyl}-13-oxa-4,10,17,21-tetraazahexacos-1-yl phosphate

A solution of N,N-diisopropylethylamine (305 mg, 2.36 mmol, 0.41 mL) and 3-ammoniopropyl 6-(benzyloxy)-6-oxohexyl phosphate (297 mg, 0.825 mmol) in N,N-dimethylformamide (5 mL) was added to a solution of 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-′7,13,20-trioxo-2,16-dioxa-8,12,19-triazatetracosan-24-oic acid (1.50 g, 0.790 mmol) in DMF (10 mL). 1-[Bis(dimethylamino)methylene]-1H-1,2,3-triazolo[4,5-b]pyridinium 3-oxid hexafluorophosphate (328 mg, 0.065 mmol) was then added to the reaction mixture at room temperature. After 1 h, the reaction was quenched with saturated ammonium chloride (30 mL) and extracted with dichloromethane (4×30 mL). The combined organic layers were washed with brine, dried over sodium sulfate, filtered and concentrated. The crude product was taken forward without further purification. LCMS (m/z) for C₉₈H₁₅₅N₁₁O₄₃PNa₂²⁺ (M+2Na)²⁺ 1125.5; retention time=0.71 min (UPLC 1.5 min method).

### Step 5: Example 29

The crude tris-benzyl ester 6-(benzyloxy)-6-oxohexyl 26-{[4,6-di-O-acetyl-2-(acetylamino)-2-deoxy-beta-D-galactopyranosyl]oxy}-5,9,16,22-tetraoxo-11,11-bis{[3-oxo-3-({3-[(5-{[3,4,6-tri-O-acetyl-2-(acetylamino)-2-deoxy-β-D-galactopyranosyl]oxy}pentanoyl)amino]propyl}amino)propoxy]methyl}-13-oxa 4,10,17,21-tetraazahexacos-1-yl phosphate (1.77 g, 0.790 mmol) was dissolved in methanol (0.05 M) and hydrogenated using an H-cube (10% Pd/C with a flow rate of 1.0 mL/min under full H₂ at 60° C.). Product was obtained cleanly and the bulk material was purified by preparatory HPLC [Column: Phenomenex Gemini XB C18 150 mm×3.0 mm 5 μm. Gradient conditions: mobile phase A=0.1% 10 mM triethylammonium acetate pH7 in water, mobile phase B=0.1% 10 mM triethylammonium acetate pH7 in acetonitrile (22-100-22% B/A, 27.0 mL/min)]. The bulk material was obtained as a white solid containing triethylammonium acetate (14 equiv. by ¹H NMR integration) (475 mg). The purity of the product was calculated to be 49 wt % and the yield was determined to be 233 mg (14%) ¹H NMR (600 MHz, CD₃OD) δ 5.34 (d, 3H), 5.07 (dd, 3H), 4.57 (d, 3H), 4.22-4.05 (m, 9H), 4.02 (t, 3H), 3.94-3.81 (m, 7H), 3.72-3.63 (m, 12H), 3.54 (dt, 3H), 3.35 (s, 6H), 3.26-3.20 (m, 17H), 3.19 (q, 84H, triethylammonium acetate), 2.43 (t, 6H), 2.30-2.17 (m, 13H), 2.14 (s, 9H), 2.03 (s, 9H), 1.95 (s, 9H), 1.93 (s, 9H), 1.93 (s, 42H, triethylammonium acetate), 1.90-1.78 (m, 4H), 1.74-1.57 (m, 22H), 1.49-1.38 (m, 2H), 1.30 (t, 126H). LCMS (m/z) for C₉₃H₁₅₄N₁₁O₄₄P²⁺ (M+2H)²⁺ 1080.5; retention time: 0.65 min (UPLC 1.5 min method).

### 18-{[27-({(2R,3R,4R,5R,6R)-3-(acetylamino)-4,5-bis(acetyloxy)-6-[(acetyloxy)methyl]tetrahydro-2H-pyran-2-yl}oxy)-12,12-bis({3-[(3-{[5-({(2R,3R,4R,5R,6R)-3-(acetylamino)-4,5-bis(acetyloxy)-6-[(acetyloxy)methyl]tetrahydro-2H-pyran-2-yl}oxy)pentanoyl]amino}propyl)amino]-3-oxopropoxy}methyl)-6,10,17,23-tetraoxo-14-oxa-5,11,18,22-tetraazaheptacosan-1-oyl]amino}-43-carboxy-18-(25-carboxy-19,19-dioxido-5-oxo-2,9,12,15,18,20-hexaoxa-6-aza-19-λ-5˜-phosphapentacos-1-yl)-37,37-dioxido-13,23-dioxo-3,6,9,16,20,27,30,33,36,38-decaoxa-12,24-diaza-37-λ˜5˜-phosphatritetracont-1-yl 5-carboxypentyl phosphate

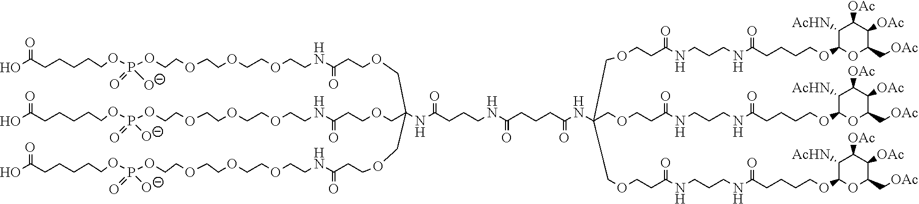 [SMILES: CC(=O)N[C@H]1[C@H](OCCCCC(=O)NCCCNC(=O)CCOCC(COCCC(=O)NCCCNC(=O)CCCCO[C@@H]2O[C@H](COC(C)=O)[C@H](OC(C)=O)[C@H](OC(C)=O)[C@H]2NC(C)=O)(COCCC(=O)NCCCNC(=O)CCCCO[C@@H]2O[C@H](COC(C)=O)[C@H](OC(C)=O)[C@H](OC(C)=O)[C@H]2NC(C)=O)NC(=O)CCCC(=O)NCCCC(=O)NC(COCCC(=O)NCCOCCOCCOCCOP(=O)([O-])OCCCCCC(=O)O)(COCCC(=O)NCCOCCOCCOCCOP(=O)([O-])OCCCCCC(=O)O)COCCC(=O)NCCOCCOCCOCCOP(=O)([O-])OCCCCCC(=O)O)O[C@H](COC(C)=O)[C@H](OC(C)=O)[C@@H]1OC(C)=O]

### Reaction Scheme:

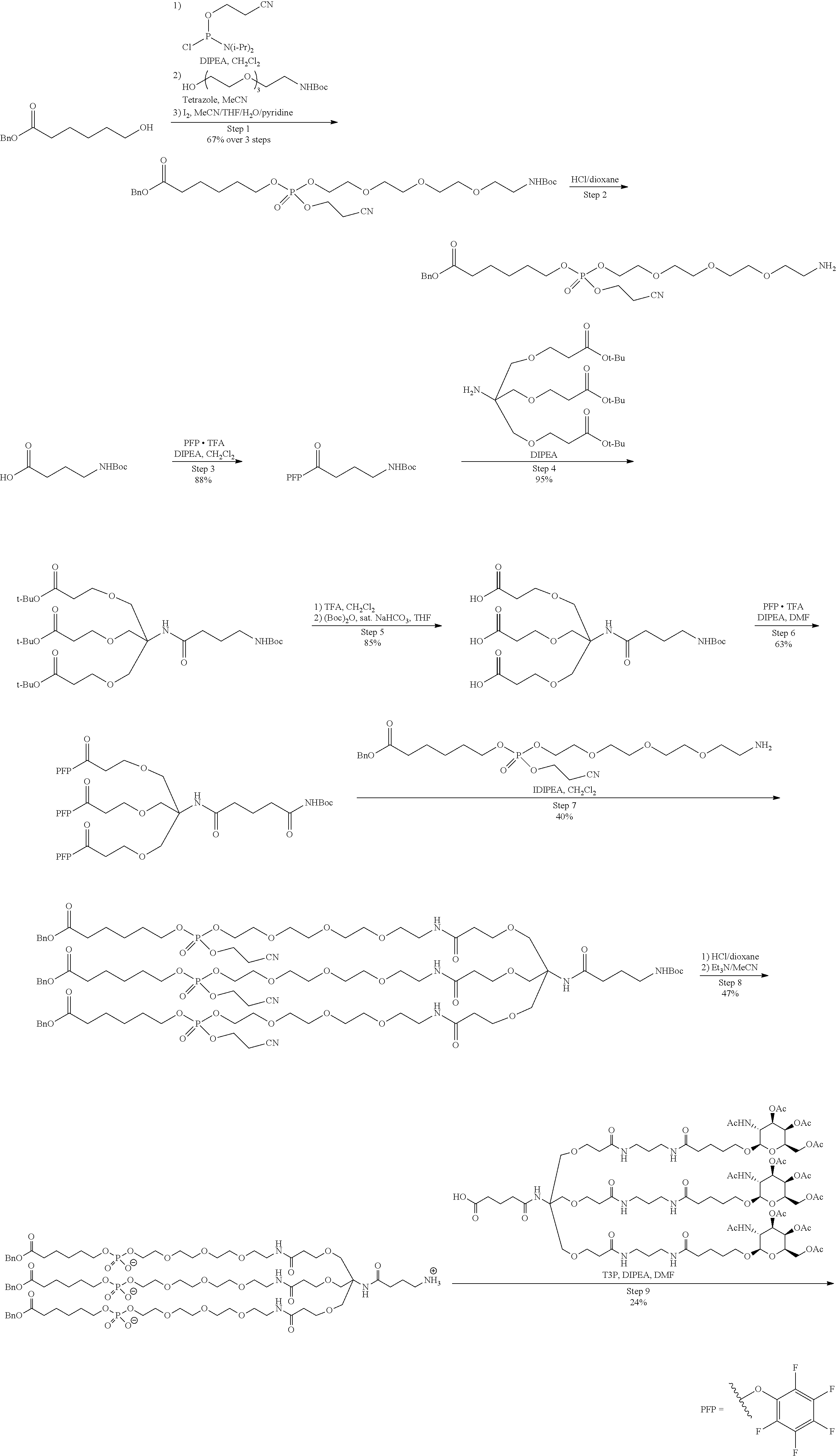

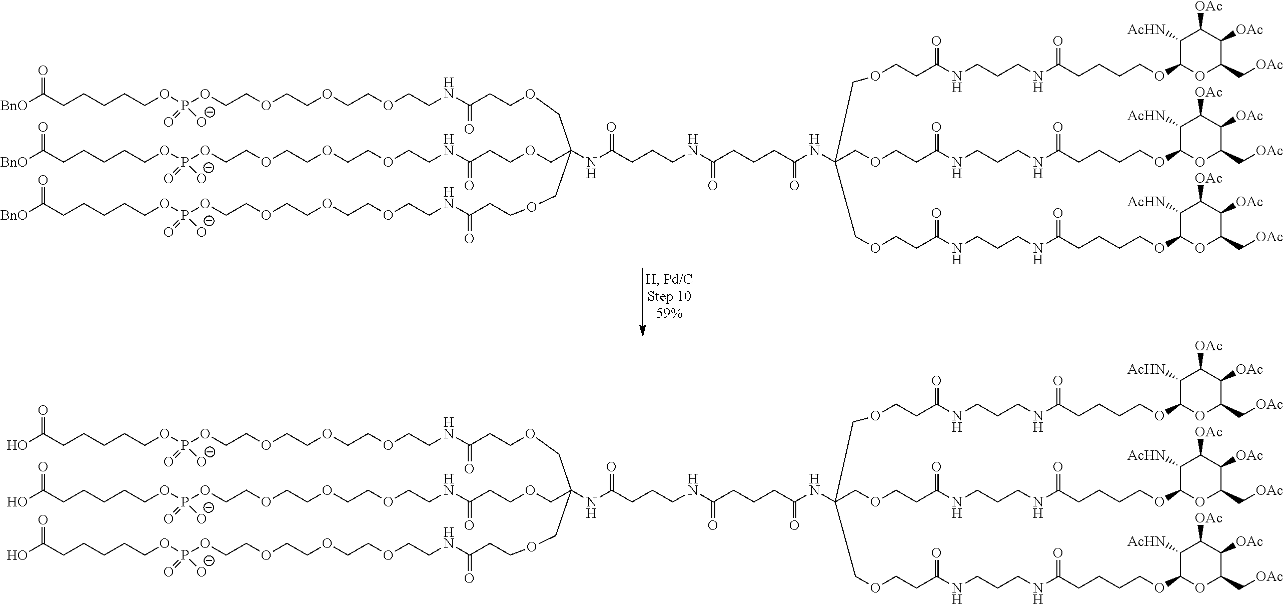

### Step 1: Benzyl 6-(((2-(2-(2-(2-aminoethoxy)ethoxy)ethoxy)ethoxy)(2-cyanoethoxy)phosphoryl)oxy)hexanoate

This compound was prepared from tert-butyl (2-(2-(2-(2-hydroxyethoxy)ethoxy)ethoxy)ethyl)carbamate (6.03 g, 20.5 mmol) and 2-cyanoethyl-N,N-diisopropylchlorophosphoramidite (6.87 mL, 30.8 mmol) and 6-Hydroxyhexanoate (6.85 g, 30.8 mmol) in an analogous manner to Example 29, Step 2. The title compound was obtained after purification by silica gel flash chromatography (50-100% ethyl acetate in heptane then 5% methanol in ethyl acetate) as a yellow oil (8.64 g, 67% over three steps). ¹H NMR (400 MHz, CDCl₃) δ ppm 7.40-7.31 (m, 5H), 5.11 (s, 2H), 5.04 (s, 1H), 4.31-4.15 (m, 4H), 4.08 (q, 2H), 3.71 (ddd, 2H), 3.69-3.58 (m, 8H), 3.53 (t, 2H), 3.31 (q, 2H), 2.77 (t, 2H), 2.37 (t, 2H), 1.76-1.63 (m, 4H), 1.44 (s, 9H), 1.48-1.36 (m, 2H). LCMS (m/z) for C₂₉H₄₇N₂NaO₁₁P⁺ (M+Na)⁺ 653.5; retention time=0.93 min (UPLC 1.5 min method).

### Step 2: 2-(2-(2-(2-((((6-(Benzyloxy)-6-oxohexyl)oxy)(2-cyanoethoxy)phosphoryl)oxy)ethoxy)ethoxy)ethoxy)ethan-1-aminium chloride

To a solution of benzyl 6-(((2-(2-(2-(2-aminoethoxy)ethoxy)ethoxy)ethoxy)(2-cyanoethoxy)phosphoryl)oxy) hexanoate (8.64 g, 14.1 mmol) in 1,4-dioxane (33 mL) at 0° C. was added a solution of hydrochloric acid (4 M in 1,4-dioxane, 87 mL, 348 mmol). The resulting mixture was stirred at ambient temperature for 1 h. The solvent was removed in vacuo to yield 8.76 g (>100%) title compound as a yellow oil. The crude product was used without further purification. ¹H NMR (400 MHz, CD₃OD) δ ppm 7.45-7.26 (m, 5H), 5.12 (s, 2H), 4.29-4.19 (m, 4H), 4.12 (q, 2H), 3.79-3.69 (m, 4H), 3.67 (m, 8H), 3.13 (t, 2H), 2.88 (ddd, 2H), 2.41 (t, 2H), 1.81-1.59 (m, 4H), 1.51-1.37 (m, 2H). LCMS (m/z) for C₂₄H₄₀N₂O₉P⁺ (M+H)⁺ 531.5; retention time=0.70 min (UPLC 1.5 min method).

### Step 3: Pentafluorophenyl 4-[(tert-butoxycarbonyl)amino]butanoate

To a solution of 4-(tert-butoxy carbonylamino)butyric acid (12.0 g, 59.0 mmol) in dichloromethane was added N,N-diisopropylethylamine (20.6 mL, 118 mmol) at ambient temperature followed by pentafluorophenyl trifluoroacetate (12.2 mL, 70.9 mmol) at 0° C. The reaction mixture was warmed to ambient temperature and stirred for 17 h. The reaction mixture was then concentrated. Purification of the crude material by silica gel flash chromatography (10-60% ethyl acetate in heptane) afforded the title compound as a white solid (19.1 g, 88%). ¹H NMR (400 MHz, CDCl₃) δ ppm 4.65 (s, 1H), 3.25 (q, 2H), 2.72 (t, 2H), 1.96 (p, 2H), 1.45 (s, 9H). LCMS (m/z) for C₁₅H₁₆F₅NNaO₄⁺ (M+Na)⁺ 392.3; retention time=1.01 min (UPLC 1.5 min method).

### Step 4: tert-Butyl 11,11-bis[(3-tert-butoxy-3-oxopropoxy)methyl]-2,2-dimethyl-4,9-dioxo-3,13-dioxa-5,10-diazahexadecan-16-oate

N,N-diisopropylethylamine (13.0 g, 101 mmol, 17.6 mL) was added to a solution of pentafluorophenyl 4-[(tert-butoxycarbonyl)amino]butanoate (9.68 g, 26.2 mmol) in THF (25 mL). tert-Butyl 3-{2-amino-3-(3-tert-butoxy-3-oxopropoxy)-2-[(3-tert-butoxy-3-oxopropoxy)methyl]propoxy}propanoate¹ (10.2 g, 20.2 mmol) was then added to the reaction in a slow stream as a solution in THF (50 mL) and the reaction was stirred at 50° C. for 78 h. The reaction was concentrated and purified twice by silica gel chromatography (0-20% methanol in dichloromethane and again 0-100% ethyl acetate in heptane) to afford the title compound as a colorless oil (13.3 g, 95%). ¹H NMR (400 MHz, CDCl₃) δ ppm 6.27-6.13 (m, 1H), 5.02-4.84 (m, 1H), 3.70 (s, 6H), 3.64 (t, 6H), 3.16 (q, 2H), 2.45 (t, 6H), 2.20 (t, 2H), 1.78 (quin, 2H), 1.45 (s, 36H). ¹ This compound was prepared according to a reported literature procedure: Cardonna, C. M.; Gawley, R. E. J. Org. Chem. 2002, 67, 1411.

### Step 5: 11,11-bis[(2-carboxyethoxy)methyl]-2,2-dimethyl-4,9-dioxo-3,13-dioxa-5,10-diazahexadecan-16-oic acid

Trifluoroacetic acid (46 g, 0.40 mol, 30 mL) was added to a solution of tert-butyl 11,11-bis[(3-tert-butoxy-3-oxopropoxy)methyl]-2,2-dimethyl-4,9-dioxo-3,13-dioxa-5,10-diazahexadecan-16-oate (13.3 g, 19.2 mmol) dichloromethane (100 mL) and the resulting solution was stirred at room temperature. After 20 h, the reaction mixture was concentrated. The resultant residue was then suspended in a mixture of tetrahydrofuran (30 mL) and saturated aqueous sodium bicarbonate (160 mL) to which was added di-tert-butyl dicarbonate (12.6 g, 57.7 mmol). The resultant suspension was heated to 40° C. Two additional aliquots of di-tert-butyl dicarbonate (3.70 g, 17.0 mmol each), were added to the reaction mixture, one at 30 min and then second at 90 min and the reaction was allowed to stir at 40° C. After 20 h, the reaction mixture was washed once with ethyl acetate and the wash was discarded. The pH of the aqueous layer was adjusted to pH=3 with 1 N hydrochloric acid. The aqueous layer was then extracted with ethyl acetate (×2) and the combined extracts were dried over magnesium sulfate, filtered, and concentrated to afford the title compound as a colorless oil which was used in the subsequent step without purification. ¹H NMR (400 MHz, CD₃OD) δ ppm 4.45-4.30 (m, 12H), 3.70 (q, 2H), 3.23 (t, 6H), 2.84 (t, 2H), 2.34 (quin, 2H), 2.18 (s, 9H).

### Step 6: Pentafluorophenyl 3-(2-[(4-aminobutanoyl)amino]-3-[3-oxo-3-(pentafluorophenoxy)propoxy]-2-{[3-oxo-3-(pentafluorophenoxy)propoxy]methyl}propoxy)propanoate

N,N-diisopropylethylamine (17.0 g, 132 mmol, 23.0 mL) was added to a solution of 11,11-bis[(2-carboxyethoxy)methyl]-2,2-dimethyl-4,9-dioxo-3,13-dioxa-5,10-diazahexadecan-16-oic acid (7.49 g, 13.0 mmol) in N,N-dimethylformamide (100 mL). Pentafluorophenyl trifluoroacetate (20.4 g, 72.7 mmol, 12.5 mL) was then added to the reaction mixture dropwise over 15 min resulting in a light pink solution that turned yellow over time. After 1 h, the reaction was quenched with saturated sodium bicarbonate. The resultant mixture was extracted with ethyl acetate (×2). The combined organic extracts were washed with brine, dried over sodium sulfate, filtered, and concentrated. The resultant residue was purified by silica gel chromatography (0-80% ethyl acetate in heptane) to afford the title compound as a colorless oil (8.76 g, 63% over 2 steps). ¹H NMR (400 MHz, (CD₃)₂SO) δ ppm 7.10 (s, 1H), 6.73 (t, 1H), 3.70 (t, 6H), 3.64 (s, 6H), 2.98 (t, 6H), 2.87 (q, 2H), 2.03 (t, 2H), 1.53 (quin, 2H), 1.36 (s, 9H).

### Step 7: Dibenzyl 27-({4-[(tert-butoxycarbonyl)amino]butanoyl}amino)-8,46-bis(2-cyanoethoxy)-27-[19-(2-cyanoethoxy)-19-oxido-5,26-dioxo-28-phenyl-2,9,12,15,18,20,27-heptaoxa-6-aza-19-λ˜5˜-phosphaoctacos-1-yl]-22,32-dioxo-7,9,12,15,18,25,29,36,39,42,45,47-dodecaoxa-21,33-diaza-8,46-diphosphatripentacontane-1,53-dioate 8,46-dioxide

Both of the starting materials were azeotroped with toluene twice and placed under high vacuum overnight prior to use. To a solution of pentafluorophenyl 3-(2-[(4-aminobutanoyl)amino]-3-[3-oxo-3-(pentafluorophenoxy)propoxy]-2-{[3-oxo-3-(pentafluorophenoxy) propoxy]methyl}propoxy)propanoate (3.97 g, 3.89 mmol) in dichloromethane (15 mL) was added N,N-diisopropylethylamine (6.8 mL, 39 mmol). Then a solution of 2-(2-(2-(2-((((6-(Benzyloxy)-6-oxohexyl)oxy)(2-cyanoethoxy)phosphoryl)oxy)ethoxy)ethoxy)ethoxy)ethan-1-aminium chloride (8.76 g crude, 14.0 mmol) in dichloromethane (25 mL) was added at 0° C. The reaction mixture was warmed to ambient temperature and stirred until TLC analysis showed consumption of starting material (15 h). The solvent was removed in vacuo, and the residue was redissolved in ethyl acetate. The solution was washed with water, saturated sodium bicarbonate and then water again, and the combined aqueous layers were extracted with ethyl acetate once. The combined organic extracts were dried with magnesium sulfate, filtered and concentrated in vacuo. The crude material was purified by silica gel flash chromatography (0-22% methanol in dichloromethane) to afford the title compound as a colorless gum (3.23 g, 40%). ¹H NMR (400 MHz, CDCl₃) δ ppm 7.38-7.30 (m, 15H), 6.71 (s, 3H), 6.52 (s, 1H), 5.19 (t, 1H), 5.11 (s, 6H), 4.32-4.14 (m, 12H), 4.08 (q, 6H), 3.76-3.60 (m, 42H), 3.55 (t, 6H), 3.43 (q, 6H), 3.14 (dd, 2H), 2.77 (t, 6H), 2.42 (t, 6H), 2.38 (t, 6H), 2.23 (t, 2H), 1.79-1.64 (m, 14H), 1.46-1.38 (m, 15H). LCMS (m/z) for C₉₄H₁₅₁F₅N₈O₃₆P₃²⁺ (M+2H)²⁺ 1031.0; retention time=1.00 min (UPLC 1.5 min method).

### Step 8: 29-[(4-Ammoniobutanoyl)amino]-29-(19,19-dioxido-5,26-dioxo-28-phenyl-2,9,12,15,18,20,27-heptaoxa-6-aza-19-λ˜5˜-phosphaoctacos-1-yl)-10,10-dioxido-3,24,34-trioxo-1-phenyl-2,9,11,14,17,20,27,31,38,41,44-undecaoxa-23,35-diaza-10-λ˜5˜-phosphahexatetracontan-46-yl 6-(benzyloxy)-6-oxohexyl phosphate. To a solution of dibenzyl 27-({4-[(tert-butoxycarbonyl)amino]butanoyl}amino)-8,46-bis(2-cyanoethoxy)-27-[19-(2-cyanoethoxy)-19-oxido-5,26-dioxo-28-phenyl-2,9,12,15,18,20,27-heptaoxa-6-aza-19-λ˜-5˜-phosphaoctacos-1-yl]-22,32-dioxo-7,9,12,15,18,25,29,36,39,42,45,47-dodecaoxa-21,33-diaza-8,46-diphosphatripentacontane-1,53-dioate 8,46-dioxide (3.22 g, 1.56 mmol) in 1,4-dioxane (18 mL) at 0° C. was added a solution of hydrochloric acid (4 M in 1,4-dioxane, 16 mL, 63 mmol). The resulting mixture was stirred at ambient temperature for 1 h. The solvent was then removed to provide an oily residue. This crude was suspended in acetonitrile (18 mL) and triethylamine (12 mL, 86 mmol) was added. The reaction mixture was stirred at ambient temperature for 40 h and subsequently concentrated in vacuo. The crude material was purified by reverse phase HPLC with a Phenomenex NX-C18 column (5-100% acetonitrile in water, containing 0.1% sodium hydroxide) and lyophilized to provide the title compound as a colorless oil (1.33 g, 47% over two steps). ¹H NMR (400 MHz, CD₃OD) δ ppm 7.41-7.27 (m, 15H), 5.11 (s, 6H), 4.03-3.92 (m, 6H), 3.85 (q, 6H), 3.71-3.58 (m, 42H), 3.55 (t, 6H), 3.38 (t, 6H), 2.99 (t, 2H), 2.44 (t, 6H), 2.42-2.29 (m, 8H), 1.92 (p, 2H), 1.72-1.57 (m, 12H), 1.48-1.37 (m, 6H). LCMS (m/z) for C₈₀H₁₃₄N₅O₃₄P3²⁺ (M+2H)²⁺ 901.5; retention time=0.77 min (UPLC 1.5 min method)

### Step 9: 29-{[27-({(2R,3R,4R,5R,6R)-3-(acetylamino)-4,5-bis(acetyloxy)-6-[(acetyloxy)methyl]tetrahydro-2H-pyran-2-yl}oxy)-12,12-bis({3-[(3-{[5-({(2R,3R,4R,5R,6R)-3-(acetylamino)-4,5-bis(acetyloxy)-6-[(acetyloxy)methyl]tetrahydro-2H-pyran-2-yl}oxy)pentanoyl]amino}propyl)amino]-3-oxopropoxy}methyl)-6,10,17,23-tetraoxo-14-oxa-5,11,18,22-tetraazaheptacosan-1-oyl]amino}-29-(19,19-dioxido-5,26-dioxo-28-phenyl-2,9,12,15,18,20,27-heptaoxa-6-aza-19-λ˜5˜-phosphaoctacos-1-yl)-10,10-dioxido-3,24,34-trioxo-1-phenyl-2,9,11,14,17,20,27,31,38,41,44-undecaoxa-23,35-diaza-10-λ˜5˜-phosphahexatetracontan-46-yl 6-(benzyloxy)-6-oxohexyl phosphate

Both of the starting materials were azeotroped with toluene three times and placed under high vacuum overnight before use. To a solution of the amine 29-[(4-ammoniobutanoyl)amino]-29-(19,19-dioxido-5,26-dioxo-28-phenyl-2,9,12,15,18,20,27-heptaoxa-6-aza-19-λ˜5˜-phosphaoctacos-1-yl)-10,10-dioxido-3,24,34-trioxo-1-phenyl-2,9,11,14,17,20,27,31,38,41,44-undecaoxa-23,35-diaza-10-λ˜5˜-phosphahexatetracontan-46-yl 6-(benzyloxy)-6-oxohexyl phosphate (270 mg, 0.147 mmol) in anhydrous dimethylformamide (0.5 mL) was added 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-′7,13,20-trioxo-2,16-dioxa-8,12,19-triazatetracosan-24-oic acid (337 mg, 0.177 mmol) in dimethylformamide (1.5 mL), N,N-diisopropylethylamine (0.21 mL, 1.18 mmol) and then Propylphosphonic anhydride solution (50 wt. % in ethyl acetate, 0.26 mL, 0.44 mmol). The reaction mixture was stirred at 50° C. for 17 h. Upon cooling to ambient temperature, water was added and the mixture was extracted with 85:15 CH₂Cl₂:i-PrOH (100 mL×3). The organic extracts were dried over sodium sulfate, filtered and concentrated in vacuo. The crude material was purified by reverse phase HPLC with a Phenomenex NX-C18 column (35-100 acetonitrile in water, containing 10 mM triethylammonium acetate) and freeze dried to provide 202 mg of the desired product containing triethylammonium acetate (12.3 equiv. based on ¹H NMR integration). The purity of this product was calculated to be 66 wt % and the yield was determined to be 133 mg (24%). This material was used in the next step without further purification. ¹H NMR (400 MHz, CD₃OD) δ ppm 7.43-7.24 (m, 15H), 5.33 (d, 3H), 5.11 (s, 6H), 5.07 (dd, 3H), 4.57 (d, 3H), 4.22-3.80 (m, 25H), 3.75-3.57 (m, 59H), 3.57-3.47 (m, 12H), 3.41-3.34 (m, 12H), 3.20 (q, 74H, triethylammonium acetate), 2.48-2.35 (m, 18H), 2.27-2.17 (m, 12H), 2.14 (s, 9H), 2.02 (s, 9H), 1.96, (s, 111H, triethylammonium acetate) 1.95 (s, 9H), 1.93 (s, 9H), 1.77-1.54 (m, 33H), 1.44 (m 9H), 1.31 (t, 111H, triethylammonium acetate). LCMS (m/z) for C₁₆₄H₂₆₆N₁₅O₇₂P₃²⁺ (M+2H)²⁺ 1845.8; retention time=1.07 min (UPLC 3 min method).

### Step 10: Example 30

A mixture of 29-{[27-({2R,3R,4R,5R,6R)-3-(acetylamino)-4,5-bis(acetyloxy)-6-[(acetyloxy)methyl]tetrahydro-2H-pyran-2-yl}oxy)-12,12-bis({3-[(3-{[5-({(2R,3R,4R,5R, 6R)-3-(acetylamino)-4,5-bis(acetyloxy)-6-[(acetyloxy)methyl]tetrahydro-2H-pyran-2-yl}oxy)pentanoyl]amino}propyl)amino]-3-oxopropoxy}methyl)-6,10,17,23-tetraoxo-14-oxa-5,11,18,22-tetraazaheptacosan-1-oyl]amino}-29-(19,19-dioxido-5,26-dioxo-28-phenyl-2,9,12,15,18,20,27-heptaoxa-6-aza-19-λ˜5˜-phosphaoctacos-1-yl)-10,10-dioxido-3,24,34-trioxo-1-phenyl-2,9,11,14,17,20,27,31,38,41,44-undecaoxa-23,35-diaza-10-5-phosphahexatetracontan-46-yl 6-(benzyloxy)-6-oxohexyl phosphate (200 mg, 0.0330 mmol, 66 wt %) and 10% palladium on carbon (7.0 mg, 0.0066 mmol) in methanol (2 mL) was stirred under hydrogen pressure (50 psi) at 25° C. for 20 h. The catalyst was filtered through 0.45 um nylon acrodisc, and washed with methanol (40 mL). The filtrate was then concentrated and the resulting oil was dissolved in 1:1 mixture of acetonitrile and water (22 mL), adjusted to pH 5.70 by hydrochloric acid (1 N). The solution was lyophilized overnight to afford the title compound as a hygroscopic white solid (13 equiv triethylamine hydrochloride salt) (110 mg, 59%). ¹H NMR (600 MHz, CD₃OD) δ ppm 5.34 (d, 3H), 5.07 (dd, 3H), 4.57 (d, 3H), 4.20-3.91 (m, 14H), 3.93-3.84 (m, 9H), 3.75-3.62 (m, 63H), 3.60-3.50 (m, 10H), 3.43-3.35 (m, 8H), 3.27-3.14 (m, 80H, triethylamine hydrochloride), 3.12-2.90 (m, 2H), 2.57-2.38 (m, 12H), 2.30-2.20 (m, 18H), 2.14 (s, 9H), 2.03 (s, 9H), 1.95-1.94 (m, 18H), 1.89-1.83 (m, 1H), 1.73-1.55 (m, 34H), 1.46-1.40 (m, 9H), 1.31 (t, 120H, triethylamine hydrochloride). LCMS (m/z) for C₁₄₃H₂₄₉N₁₅O₇₂P₃³⁺ (M+3H)³⁺ 1141.2; retention time=1.06 min (UPLC 3 min method).

### 1-[(1S,2R,3R,4R,5S)-4-(acetylamino)-2,3-bis(acetyloxy)-6,8-dioxabicyclo[3.2.1]oct-1-yl]-18,18-bis{17-[(1S,2R,3R,4R,5S)-4-(acetylamino)-2,3-bis(acetyloxy)-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oic acid

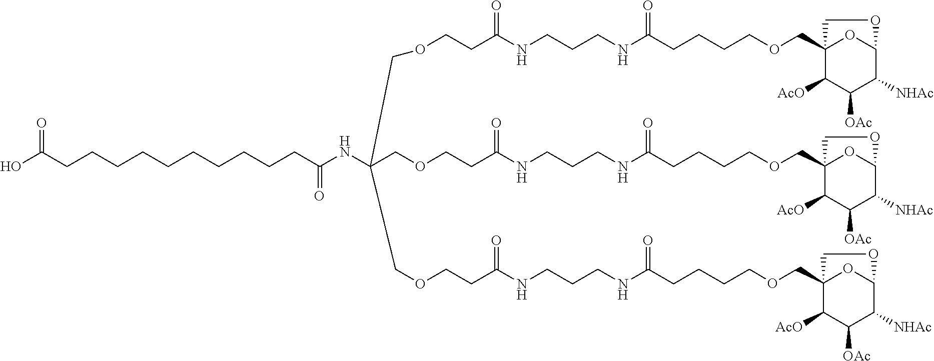 [SMILES: CC(=O)N[C@H]1[C@H]2OC[C@](COCCCCC(=O)NCCCNC(=O)CCOCC(COCCC(=O)NCCCNC(=O)CCCCOC[C@@]34CO[C@@H](O3)[C@H](NC(C)=O)[C@@H](OC(C)=O)[C@H]4OC(C)=O)(COCCC(=O)NCCCNC(=O)CCCCOC[C@@]34CO[C@@H](O3)[C@H](NC(C)=O)[C@@H](OC(C)=O)[C@H]4OC(C)=O)NC(=O)CCCCCCCCCCC(=O)O)(O2)[C@H](OC(C)=O)[C@@H]1OC(C)=O]

### Synthetic Scheme:

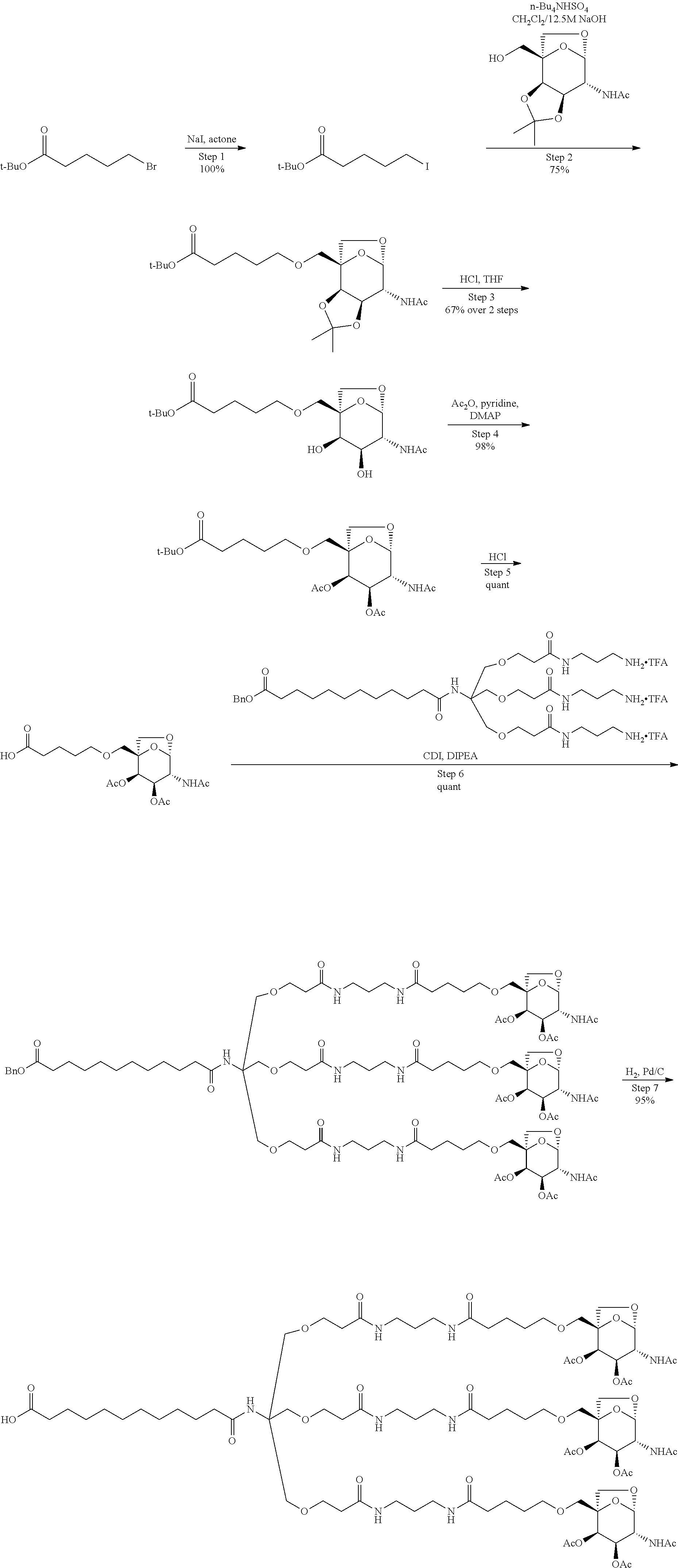

### Step 1: tert-Butyl 5-iodopentanoate

To a solution of tert-butyl 5-bromopentanoate (60.0 g, 250 mmol) in acetone (400 mL) was added sodium iodide (94.8 g, 633 mmol). The reaction mixture was stirred at 57° C. for 4 h, cooled to room temperature, filtered and washed with dichloromethane. The solvent was evaporated under reduced pressure to give a residue which was dissolved dichloromethane, washed with saturated sodium bicarbonate (200 mL) and brine (100 mL). The separated organic phase was dried over sodium sulfate, filtered, and concentrated to afford the title compound as a yellow oil (69.3 g, 100%). ¹H NMR (600 MHz, CDCl₃) δ ppm 3.20 (t, 2H), 2.25 (t, 2H), 1.86 (p, 2H), 1.70 (p, 2H), 1.45 (s, 9H).

### Step 2: tert-Butyl 5-{[(1S,2R,6R,7R,8S)-7-(acetylamino)-4,4-dimethyl-3,5,9,11-tetraoxatricyclo[6.2.1.0˜2,6˜]undec-1-yl]methoxy}pentanoate

To a solution of tert-butyl 5-iodopentanoate (59 g, 0.21 mol) and N-((3aR,4S,7S,8R,8aR)-4-(hydroxymethyl)-2,2-dimethylhexahydro-4,7-epoxy[1,3]dioxolo[4,5-d]oxepin-8-yl)acetamide (20 g, 69 mmol) in dichloromethane (210 mL) was added tetrabutylammonium hydrogensulfate (35.3 g, 104 mmol) followed by 12.5 M sodium hydroxide solution (160 mL) in an ice bath. The reaction mixture was stirred at room temperature for 24 h. The reaction mixture was partitioned between dichloromethane (200 mL) and water (200 mL). The separated organic phase was washed by 1 N hydrochloric acid (300 mL), dried over sodium sulfate, filtered, and concentrated. The crude was triturated in diethyl ether (500 mL) at ambient temperature for 30 min. The resultant solid was removed by filtration and the filter cake was rinsed by diethyl ether (100 mL). The filtrate was concentrated, and dried in vacuo overnight to afford the crude of the title compound as a yellow oil (50.9 g, 45.5 wt % pure determined by qNMR with 1,3,5-trimethoxyben as the internal standard) which was used in next step without purification. LCMS (m/z) for C₂₁H₃₆NO₈⁺ (M+H)⁺ 430.3; retention time=0.88 min (UPLC 1.5 min method).

### Step 3: tert-Butyl 5-{[(1S,2R,3R,4R,5S)-4-(acetylamino)-2,3-dihydroxy-6,8-dioxabicyclo[3.2.1]oct-1-yl]methoxy}pentanoate

To an ice cold solution of the crude of tert-butyl 5-{[(1S,2R,6R,7R,8S)-7-(acetylamino)-4,4-dimethyl-3,5,9,11-tetraoxatricyclo[6.2.1.0˜2,6˜]undec-1-yl]methoxy}pentanoate (50.9 g, 45.5 wt %, 53.9 mmol) in tetrahydrofuran (105 mL) was added a solution of concentrated hydrochloric acid (16 mL) in water (49 mL) via addition funnel over 5 min. The reaction solution was stirred at room temperature under nitrogen for 6 h. The reaction mixture was diluted with 2-methyl-tetrahydrofuran (300 mL) and washed with brine (100 mL). The aqueous phase was extracted with dichloromethane (300 mL). Each separated organic phase was washed by a mixture of saturated sodium bicarbonate (75 mL) and brine (75 mL), then brine (120 mL). The organic phases were combined, dried over sodium sulfate, filtered, concentrated, and azeotroped by heptane (100 mL) followed by methyl-t-butyl-ether (100 mL). The resulting crude was triturated in methyl-t-butyl-ether (200 mL) at room temperature for 15 min. The resulting precipitate was collected by filtration, rinsed with methyl-t-butyl-ether (200 mL), and dried in vacuo to afford the title compound as white solid (17.9 g, 66% over 2 steps). ¹H NMR (400 MHz, CDCl₃) δ ppm 5.79 (d, 1H), 5.36 (d, 1H), 4.07-3.88 (m, 4H), 3.79-3.62 (m, 4H), 3.60-3.46 (m, 2H), 3.37 (d, 1H), 2.30-2.19 (m, 2H), 2.07 (s, 3H), 1.75-1.51 (m, 4H), 1.45 (s, 9H). LCMS (m/z) for C₁₈H₃₂NO₈⁺ (M+H)⁺ 390.5; retention time=0.70 min (UPLC 1.5 min method).

### Step 4: tert-Butyl 5-{[(1S,2R,3R,4R,5S)-4-(acetylamino)-2,3-bis(acetyloxy)-6,8-dioxabicyclo[3.2.1]oct-1-yl]methoxy}pentanoate

Acetic anhydride (18.7 g, 183 mmol) was added dropwise to an ice cold solution of tert-butyl 5-{[(1S,2R,3R,4R,5S)-4-(acetylamino)-2,3-dihydroxy-6,8-dioxabicyclo[3.2.1]oct-1-yl]methoxy}pentanoate (18.6 g, 49.7 mmol) and pyridine (14.0 g, 183 mmol), and dimethylaminopyridine (1.12 g, 9.16 mmol) in dichloromethane (150 mL). The mixture was stirred at room temperature for 2.5 h. The reaction mixture was quenched by hydrochloric acid (1 N, 150 mL) and extracted with dichloromethane (200 mL). The organic phase was washed with hydrochloric acid (1 N, 150 mL), saturated sodium bicarbonate (150 mL) dried over sodium sulfate, concentrated, and azeotroped with heptane (4×100 mL) to the crude which was purified by a silica gel plug (210 g silica gel, 100% heptane (1 L), then 25% ethyl acetate in heptane (2 L), followed by 100% ethyl acetate (2 L)) to afford the title compound as white solid (21.2 g, 98%). ¹H NMR (400 MHz, CDCl₃) δ ppm 5.65 (d, 1H), 5.41-5.39 (m, 2H), 5.09 (dd, 1H), 4.34 (t, 1H), 3.93 (d, 1H), 3.75 (d, 1H), 3.65 (d, 1H), 3.50 (d, 1H), 3.45 (td, 1H), 3.38 (td, 1H), 2.21 (t, 2H), 2.17 (s, 3H), 2.00 (s, 3H), 1.98 (s, 3H), 1.64-1.52 (m, 4H), 1.44 (s, 9H). LCMS (m/z) for C₂₂H₃₅NNaO₁₀⁺ (M+Na)⁺ 496.1; retention time=0.85 min (UPLC 1.5 min method).

### Step 5: 5-{[(1S,2R,3R,4R,5S)-4-(acetylamino)-2,3-bis(acetyloxy)-6,8-dioxabicyclo[3.2.1]oct-1-yl]methoxy}pentanoic acid

To a solution of tert-butyl 5-{[(1S,2R,3R,4R,5S)-4-(acetylamino)-2,3-bis(acetyloxy)-6,8-dioxabicyclo[3.2.1]oct-1-yl]methoxy}pentanoate (21.1 g, 44.6 mmol) in dichloromethane (110 mL) placed in an ice bath was added hydrochloric acid (11 g, 0.31 mol, 78 mL, 4.0 M in 1,4-dioxane). The cooling bath was removed and the reaction was stirred at ambient temperature under nitrogen for 4 h. The reaction mixture was concentrated, and azeotroped with diethyl ether (200 mL), ethyl acetate (200 mL), and heptane (3×200 mL), and finally dried by vacuum overnight to afford the title compound as a white solid (18.6 g, quantitative). ¹H NMR (400 MHz, CD₃CN) δ ppm 8.84 (br.s., 1H), 6.50 (d, 1H), 5.35 (d, 1H), 5.30 (d, 1H), 5.00 (dd, 1H), 4.17-4.07 (m, 1H), 3.93 (d, 1H), 3.78-3.56 (m, 2H), 3.52 (d, 1H), 3.49-3.34 (m, 2H), 2.26 (t, 2H), 2.12 (s, 3H), 1.92 (s, 3H), 1.85 (s, 3H), 1.60-1.47 (m, 4H). LCMS (m/z) for C₁₈H₂₈NO₁₀⁺ (M+H)⁺ 418.0; retention time=0.60 min (UPLC 1.5 min method).

### Step 6: Benzyl 1-[(1S,2R,3R,4R,5S)-4-(acetylamino)-2,3-bis(acetyloxy)-6,8-dioxabicyclo[3.2.1]oct-1-yl]-18,18-bis{17-[(1S,2R,3R,4R,5S)-4-(acetylamino)-2,3-bis(acetyloxy)-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oate

To a solution of 5-{[(1S,2R,3R,4R,5S)-4-(acetylamino)-2,3-bis(acetyloxy)-6,8-dioxabicyclo[3.2.1]oct-1-yl]methoxy}pentanoic acid (1.53 g, 3.63 mmol) in acetonitrile (6 mL) was added 1,1′-carbonyldiimidazole (0.580 g, 3.56 mmol) and the reaction mixture was stirred at room temperature. After 3 h, benzyl 12-((1,19-diamino-10-((3-((3-aminopropyl)amino)-3-oxopropoxy)methyl)-5,15-dioxo-8,12-dioxa-4,16-diazanonadecan-10-yl)amino)-12-oxododecanoate tris-trifluoroacetate salt (1.58 g, 1.03 mmol, 75.4 wt %) was added to the reaction mixture as a solution in acetonitrile (6 mL) followed by N,N-diisopropylethylamine (0.540 g, 4.14 mmol). The reaction mixture was stirred at room temperature overnight. The reaction mixture was concentrated, diluted by dichloromethane (70 mL), washed by hydrochloric acid (1 N, 30 mL), brine (30 mL), and saturated sodium bicarbonate (30 mL). The organic phase was dried over sodium sulfate, filtered, and concentrated. The resultant residue was purified by a silica gel plug (20 g silica gel, eluted with dichloromethane (100 mL), 10% methanol in dichloromethane (200 mL), followed by 25% methanol in dichloromethane (200 mL)) to afford the title compound as a white glass (2.25 g, quantitative yield). ¹H NMR (600 MHz, CD₃OD) δ ppm δ 7.73-7.30 (m, 5H), 5.44 (d, 3H), 5.32 (s, 3H), 5.12-5.09 (m, 5H), 4.18 (d, 3H), 4.00 (d, 3H), 3.71 (dd, 6H), 3.68-3.67 (m, 12H), 3.51-3.45 (m, 6H), 3.41-3.38 (m, 3H), 3.21 (q, 12H), 2.42 (t, 6H), 2.35 (t, 2H), 2.22-2.17 (m, 6H), 2.16 (s, 9H), 1.94 (s, 18H), 1.72-1.58 (m, 16H), 1.58-1.42 (m, 8H), 1.38-1.29 (m, 12H). LCMS (m/z) for C₉₅H₁₅₀N₁₀O₃₆²⁺ (M+2H)²⁺ 1004.7; retention time=0.86 min (UPLC 3 min method)

### Step 7: Example 31

A mixture of benzyl 1-[(1S,2R,3R,4R,5S)-4-(acetylamino)-2,3-bis(acetyloxy)-6,8-dioxabicyclo[3.2.1]oct-1-yl]-18,18-bis{17-[(1S,2R,3R,4R,5S)-4-(acetylamino)-2,3-bis(acetyloxy)-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oate (2.36 g, 1.18 mmol) and 10% palladium on carbon (0.376 mg) in methanol (14 mL) was stirred under hydrogen pressure (50 psi) at 25° C. in a stirred Parr reactor for 4.5 h. The reaction mixture was filtered through celite to remove the catalyst. The celite was washed with methanol (20 mL) and the combined filtrates were concentrated and azeotroped with methyl tert-butyl ether (3×20 mL). The crude was triturated in methyl tert-butyl ether (20 mL) overnight. The resulting white solid was collected by filtration, dried in vacuo to afford the title compound as a white solid (2.14 g, 95%). ¹H NMR (600 MHz, CD₃OD) δ ppm 5.45 (d, 3H), 5.32 (s, 3H), 5.11 (dd, 3H), 4.18 (d, 3H), 4.01 (d, 3H), 3.72 (dd, 6H), 3.70-3.64 (m, 12H), 3.52-3.47 (m, 6H), 3.42-3.39 (m, 3H), 3.22 (q, 12H), 2.42 (t, 6H), 2.28 (t, 2H), 2.19 (t, 6H) 2.16 (s, 9H) 1.95 (s, 18H) 1.73-1.50 (m, 24H) 1.38-1.29 (m, 12H). LCMS (m/z) for C₈₈H₁₄₃N₁₀O₃₆⁺1916.2 (M+H)⁺; retention time=1.35 min (UPLC 3 min method).

### 1-[(1S,2R,3R,4R,5S)-4-(acetylamino)-2,3-bis(acetyloxy)-6,8-dioxabicyclo[3.2.1]oct-1-yl]-18-{17-[(1S,2R,3R,4R,5S)-4-(acetylamino)-2,3-bis(acetyloxy)-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oic acid

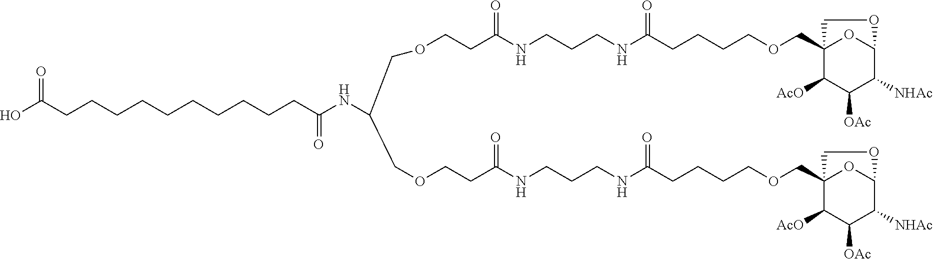 [SMILES: CC(=O)N[C@H]1[C@H]2OC[C@](COCCCCC(=O)NCCCNC(=O)CCOCC(COCCC(=O)NCCCNC(=O)CCCCOC[C@@]34CO[C@@H](O3)[C@H](NC(C)=O)[C@@H](OC(C)=O)[C@H]4OC(C)=O)NC(=O)CCCCCCCCCCC(=O)O)(O2)[C@H](OC(C)=O)[C@@H]1OC(C)=O]

### Reaction Scheme:

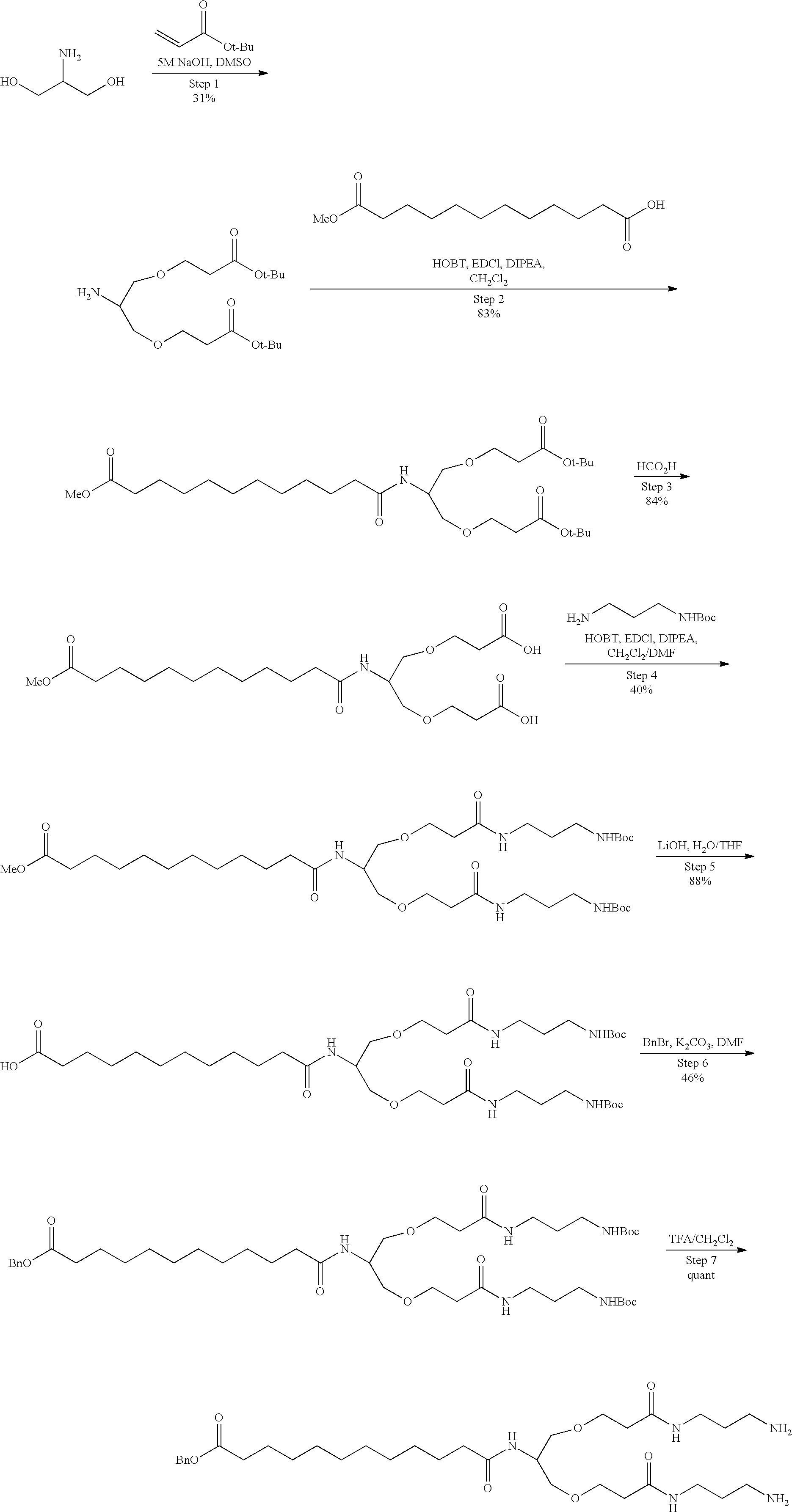

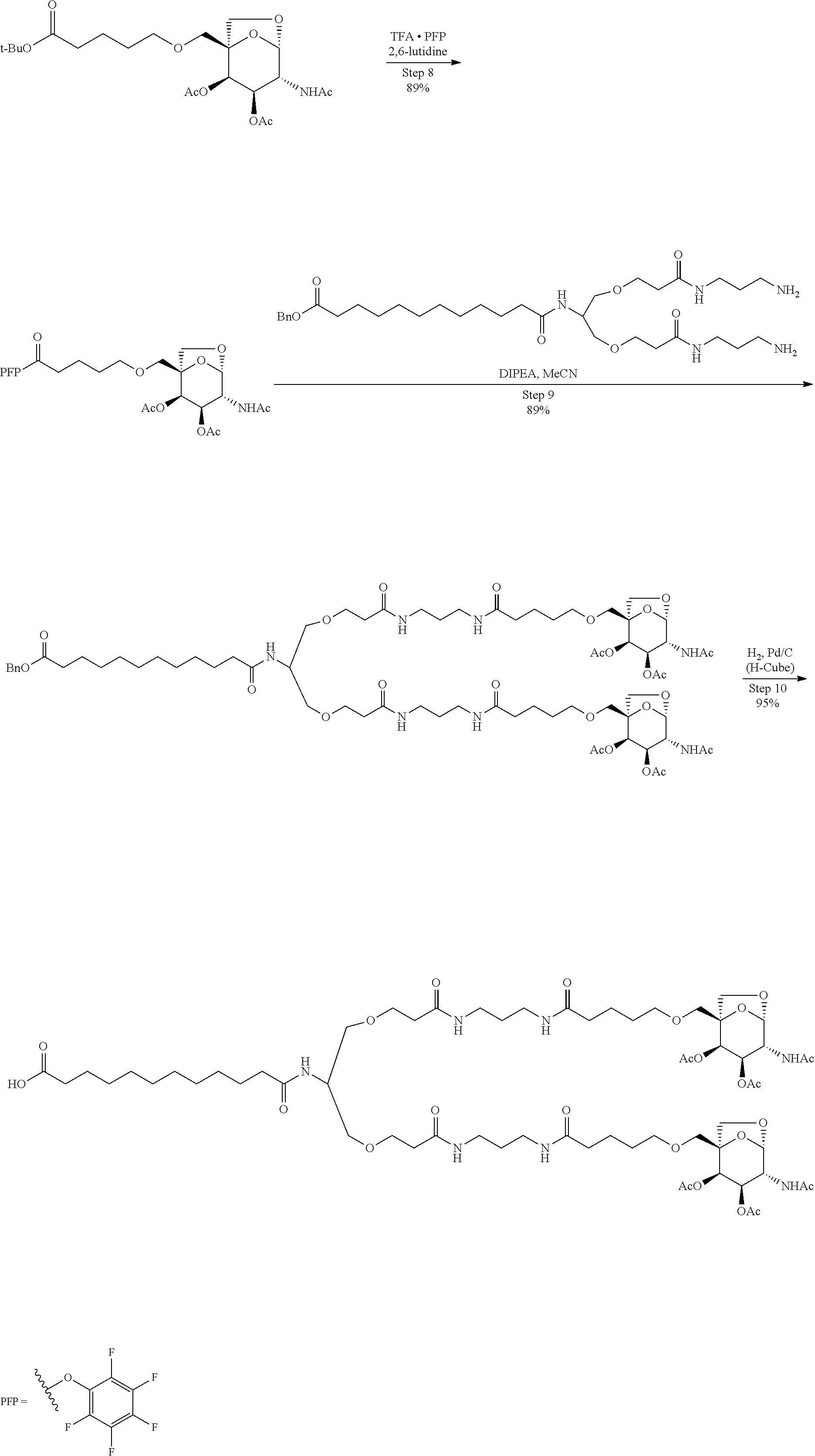

### Step 1: di-tert-Butyl 3,3′-[(2-aminopropane-1,3-diyl)bis(oxy)]dipropanoate

1,1-Dimethylethyl 2-propenoate (1.44 kg, 11.3 mol) was added to a stirred suspension of 2-amino-1,3-propanediol (500 g, 5.49 mol) in dimethylsulfoxide (1.5 L) dropwise over 1 hour at −5° C. The reaction mixture was then allowed to warm to 25° C. and stirring at that temperature continued until TLC analysis showed consumption of the starting material (16 h). The reaction mixture was diluted with water (3 L) and the mixture was extracted with ethyl acetate (5 L×1, 2.5 L×2). The combined organic layers were dried over sodium sulfate, filtered and concentrated in vacuo to afford a residue (1.30 kg). The crude was purified by column chromatography (5% ethyl acetate in petroleum ether then 10% methanol in dichloromethane) to afford the title compound as yellow oil (600 g, 31%). ¹H NMR (400 MHz, CDCl₃) δ ppm 3.69-3.60 (m, 4H), 3.45-3.37 (m, 2H), 3.29 (dd, 2H), 3.18-3.00 (m, 1H), 2.44 (t, 4H), 1.42 (s, 18H).

### Step 2: Methyl 12-oxo-12-[(2,2,16,16-tetramethyl-4,14-dioxo-3,7,11,15-tetraoxaheptadecan-9-yl)amino]dodecanoate

This reaction was carried out 2 batches in parallel. 1,12-Dodecanedioic acid monomethyl ester (128 g, 0.524 mmol), hydroxybenzotriazole (70.7 g, 0.524 mmol), diisopropylethylamine (271 g, 2.10 mol) and 1-(3-dimethylaminopropyl)-3-ethylcarbodiimide hydrochloride (201 g, 1.05 mol) were added in to a stirred solution of di-tert-Butyl 3,3′-[(2-aminopropane-1,3-diyl)bis(oxy)]dipropanoate (182 g, 0.524 mol) in dichloromethane (1.6 L) at 20° C. and the reaction was stirred at room temperature until TLC analysis showed consumption of starting material (12 h). The two batches of this reaction mixture were combined, diluted with water (2 L) and stirred at room temperature for 10 minutes. The organic layer was separated and dried over sodium sulfate, filtered and concentrated. The resultant crude residue was purified by silica gel chromatography (20-50% ethyl acetate in petroleum ether) to afford the title compound as a light yellow oil (500 g, 83%). ¹H NMR (400 MHz, CDCl₃) δ ppm 6.21 (d, 1H), 4.14-4.03 (m, 1H), 3.68-3.54 (m, 7H), 3.49 (dd, 2H), 3.32 (dd, 2H), 2.49-2.30 (m, 4H), 2.21 (t, 2H), 2.10 (t, 2H), 1.53 (br.s., 4H), 1.37 (s, 18H), 1.09-1.27 (m, 12H)

### Step 3: 3,3′-[{2-[(12-Methoxy-12-oxododecanoyl)amino]propane-1,3-diyl}bis(oxy)]dipropanoic acid

A solution of methyl 12-oxo-12-[(2,2,16,16-tetramethyl-4,14-dioxo-3,7,11,15-tetraoxaheptadecan-9-yl)amino]dodecanoate (546 g, 0.950 mol) in formic acid (2.5 L) was stirred at 30-35° C. until TLC analysis showed consumption of starting material (18 h). The mixture was concentrated to afford a crude residue which was triturated in petroleum ether/ethyl acetate (10:1, 1.5 L) at 20° C. for 12 h. The resultant slurry was filtrated and the filter cake was dried in vacuo to afford the title compound as a white solid (370 g, 84%). ¹H NMR (400 MHz, CDCl₃) δ ppm 10.02 (br.s., 2H), 6.33 (d, 1H), 4.27-4.16 (m, 1H), 3.73 (t, 4H), 3.66 (s, 3H), 3.59 (dd, 2H), 3.45 (dd, 2H), 2.59 (t, 4H), 2.30 (t, 2H), 2.20 (t, 2H), 1.66-1.53 (m, 4H), 1.26 (br.s., 12H).

### Step 4: Methyl 15-(13,13-dimethyl-5,11-dioxo-2,12-dioxa-6,10-diazatetradec-1-yl)-2,2-dimethyl-4,10,17-trioxo-3,13-dioxa-5,9,16-triazaoctacosan-28-oate

1-(3-Dimethylaminopropyl)-3-ethylcarbodiimide hydrochloride (348 g, 1.82 mol), hydroxybenzotriazole (205 g, 1.52 mol), and diisopropylethylamine (470 g, 3.64 mol) were added to a stirred solution of 3,3′-[{2-[(12-methoxy-12-oxododecanoyl)amino]propane-1,3-diyl}bis(oxy)]dipropanoic acid (280 g, 0.606 mmol) in DCM/DMF (2 L/250 mL) at 0-5° C. tert-Butyl (3-aminopropyl)carbamate (243 g, 1.39 mol) was then added to the reaction mixture at 0-5° C. in 4 portions over 20 min. The reaction was then allowed to warm to 25° C. and stirring was continued at that temperature until TLC analysis showed consumption of the starting material (12 h). The reaction mixture was concentrated and the resultant residue diluted with water (2 L). Then the mixture was extracted with ethyl acetate (2 L×1, 700 mL×2) and the combined organic layers were dried over sodium sulfate, filtered and concentrated. The resultant residue was purified by silica gel chromatography (100% ethyl acetate followed by 10% methanol in dichloromethane) to afford a white solid which was triturated in petroleum ether/ethyl acetate (1:2, 1 L) at 15° C. After 16 h the slurry was filtered and the filter cake was washed with ethyl acetate (200 mL) and subsequently dried in vacuo. The resultant solid was once again was triturated in petroleum ether/ethyl acetate (1:3, 600 mL) at 15° C. for 24 h. The slurry was filtered and the filter cake was washed with ethyl acetate (200 mL). The cake was dried in vacuo to obtain the title compound as a white solid (190 g, 40%). ¹H NMR (400 MHz, (CD₃)₂SO) δ ppm 7.81 (t, 2H), 7.63 (d, 1H), 6.76 (t, 2H), 4.01-3.85 (m, 1H), 3.64-3.48 (m, 7H), 3.41-3.22 (m, 4H), 3.02 (q, 4H), 2.90 (q, 4H), 2.28 (t, 6H), 2.05 (t, 2H), 1.57-1.43 (m, 8H), 1.37 (s, 18H), 1.22 (br.s., 12H).

### Step 5: 15-(13,13-dimethyl-5,11-dioxo-2,12-dioxa-6,10-diazatetradec-1-yl)-2,2-dimethyl-4,10,17-trioxo-3,13-dioxa-5,9,16-triazaoctacosan-28-oic acid

A solution of lithium hydroxide monohydrate (35.7 g, 853 mmol) in water (400 mL) was added to a stirred solution of methyl 15-(13,13-dimethyl-5,11-dioxo-2,12-dioxa-6,10-diazatetradec-1-yl)-2,2-dimethyl-4,10,17-trioxo-3,13-dioxa-5,9,16-triazaoctacosan-28-oate (220 g, 284 mmol) in tetrahydrofuran (1.2 L) at 20° C. The reaction mixture was then heated to 28° C. until TLC analysis showed consumption of starting material (18 h). The reaction mixture was concentrated and the resultant residue was diluted with water (2 L). The mixture was then washed with dichloromethane (1 L×2) and was acidified with aqueous hydrochloric acid (1 N, 900 mL) to pH<4. The mixture was extracted with dichloromethane (1 L×2) and the combined organic layers were dried over sodium sulfate, filtered and concentrated to afford the title compound as a light yellow gum (190 g, 88%). ¹H NMR (400 MHz, (CD₃)₂SO) δ ppm 11.97 (br.s., 1H), 7.81 (t, 2H), 7.62 (d, 1H), 6.75 (t, 2H), 3.98-3.87 (m, 1H), 3.56 (t, 4H), 3.38-3.24 (m, 4H), 3.02 (q, 4H), 2.90 (q, 4H), 2.28 (t, 4H), 2.18 (t, 2H), 2.05 (t, 2H), 1.57-1.41 (m, 8H), 1.37 (s, 18H), 1.23 (br.s., 12H).

### Step 6: Benzyl 15-(13,13-dimethyl-5,11-dioxo-2,12-dioxa-6,10-diazatetradec-1-yl)-2,2-dimethyl-4,10,17-trioxo-3,13-dioxa-5,9,16-triazaoctacosan-28-oate

Potassium carbonate (107 g, 773 mmol) and benzyl bromide (52.9 g, 309 mmol) were added to a stirred solution of 15-(13,13-dimethyl-5,11-dioxo-2,12-dioxa-6,10-diazatetradec-1-yl)-2,2-dimethyl-4,10,17-trioxo-3,13-dioxa-5,9,16-triazaoctacosan-28-oic acid (196 g, 258 mmol) in N,N-dimethylformamide (800 mL) at 20° C. The reaction mixture was then heated to 35° C. until TLC analysis showed consumption of starting material (12 h). The mixture was diluted with water (1.5 L) and extracted with methyl tert-butyl ether (1 L×1, 500 mL×2). The combined organic layers were washed with 5% aqueous lithium chloride (800 mL×2), dried over sodium sulfate, filtered and concentrated. The resultant residue was first purified by silica gel chromatography (10% methanol in dichloromethane) and subsequently by preparative HPLC (prepL-LD, Phenomenex Luna C18 250*80 mm*10 um, 30-100% acetonitrile in water modified with 10 mM ammonium bicarbonate, 250 mL/min) to afford the title compound as a white solid (100 g, 46%). ¹H NMR (400 MHz, CDCl₃) δ ppm 7.41-7.28 (m, 5H), 6.86 (t, 2H), 6.61 (d, 1H), 5.21 (t, 2H), 5.11 (s, 2H), 4.26-4.14 (m, 1H), 3.81-3.72 (m, 2H), 3.72-3.63 (m, 2H), 3.58 (dd, 2H), 3.41 (dd, 2H), 3.36-3.24 (m, 4H), 3.16 (q, 4H), 2.52-2.38 (m, 4H), 2.35 (t, 2H), 2.19 (t, 2H), 1.68-1.55 (m, 8H), 1.43 (s, 18H), 1.34-1.20 (m, 12H).

### Step 7: Benzyl 12-[(1,3-bis{3-[(3-aminopropyl)amino]-3-oxopropoxy}propan-2-yl)amino]-12-oxododecanoate

A stirred solution of benzyl 15-(13,13-dimethyl-5,11-dioxo-2,12-dioxa-6,10-diazatetradec-1-yl)-2,2-dimethyl-4,10,17-trioxo-3,13-dioxa-5,9,16-triazaoctacosan-28-oate (5.0 g, 5.9 mmol) in dichloromethane (35 mL), was cooled in an ice bath. Trifluoroacetic acid (8.8 mL, 0.12 mol) was added drop-wise over 15 minutes. After an additional 15 minutes, the ice bath was removed and the reaction stirred at ambient temperature for 2 hours. The reaction was concentrated and acetonitrile (20 mL) was added. The solution was concentrated and dried on high vacuum pump overnight. The resulting oil was dissolved in dichloromethane (125 mL) and MP-Carbonate resin (Biotage) (18 g, 3.1 mmol/g) was added to free base the material. The mixture was stirred for 2.5 hours at ambient temperature under nitrogen. The resin was then filtered off and washed with dichloromethane (25 mL) followed by methanol (25 mL). The combined filtrates were concentrated and dried in vacuo overnight to afford the title compound as a white solid (4.1 g, quantitative). ¹H NMR (400 MHz, CD₃OD) δ ppm 7.40-7.28 (m, 5H), 5.11 (s, 2H), 4.17-4.06 (m, 1H), 3.70 (t, 4H), 3.46 (d, 4H), 3.30-3.28 (m, 4H, overlapping with methanol shift), 2.95 (t, 4H), 2.46 (t, 4H), 2.36 (t, 2H), 2.20 (t, 2H), 1.85 (m, 4H), 1.61 (d, 4H), 1.30 (br.s., 12H).

### Step 8: Pentafluorophenyl 5-{[(1S,2R,3R,4R,5S)-4-(acetylamino)-2,3-bis(acetyloxy)-6,8-dioxabicyclo[3.2.1]oct-1-yl]methoxy}pentanoate

To a solution of tert-butyl 5-{[(1S,2R,3R,4R,5S)-4-(acetylamino)-2,3-bis(acetyloxy)-6,8-dioxabicyclo[3.2.1]oct-1-yl]methoxy}pentanoate (0.990 g, 2.04 mmol) in dichloromethane (8 mL) placed in an ice bath was added trifluoroacetic acid (1.90 g, 170 mmol) and the reaction was stirred at ambient temperature under nitrogen overnight. Deprotection of ester was complete as confirmed by LCMS [(m/z) for C₁₈H₂₈NO10⁺ (M+H)⁺ 418.0]. The reaction mixture was cooled to 0° C. and 2,6-lutidine (2.18 g, 20.5 mmol) followed by pentafluorophenyl trifluoroacetate (0.86 g, 3.06 mmol mL) were added sequentially via addition funnel and the reaction mixture was stirred at ambient temperature. After 3 h, the reaction was quenched by addition of hydrochloric acid (1 N, 150 mL) and extracted with dichloromethane (200 mL). The organic phase was washed with hydrochloric acid (1 N, 3×150 mL) and saturated sodium bicarbonate (150 mL), then subsequently dried over sodium sulfate, filtered, and concentrated. The crude residue was purified by silica gel chromatography (25-75% ethyl acetate in heptane) to afford the title compound as white solid (1.03 g, 87%). ¹H NMR (600 MHz, CD₃CN) δ ppm 6.49 (d, 1H), 5.36 (d, 1H), 5.30 (s, 1H), 5.01 (dd, 1H), 4.10 (t, 1H), 3.94 (d, 1H), 3.69 (dd, 2H), 3.55-3.46 (m, 2H), 3.46-3.36 (m, 1H), 2.72 (t, 2H), 2.12 (s, 3H), 1.91 (s, 3H), 1.85 (s, 3H), 1.80-1.68 (m, 2H), 1.66-1.53 (m, 2H). LCMS (m/z) for C₂₄H₂₇F₅NO₁₀⁺ (M+H)⁺ 584.8; Retention time=0.94 min (UPLC 1.5 min method).

### Step 9: Benzyl 1-[(1S,2R,3R,4R,5S)-4-(acetylamino)-2,3-bis(acetyloxy)-6,8-dioxabicyclo[3.2.1]oct-1-yl]-18-{17-[(1S,2R,3R,4R,5S)-4-(acetylamino)-2,3-bis(acetyloxy)-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oate

To a suspension of benzyl 12-[(1,3-bis{3-[(3-aminopropyl)amino]-3-oxopropoxy}propan-2-yl)amino]-12-oxododecanoate (1.05 g, 1.31 mmol) in a mixture of acetonitrile (18 mL) and dimethylformamide (8 mL) was added N,N-diisopropylethylamine (1.83 ml, 10.5 mmol). The resultant mixture was then added to a solution of pentafluorophenyl 5-{[(1S,2R,3R,4R,5S)-4-(acetylamino)-2,3-bis(acetyloxy)-6,8-dioxabicyclo[3.2.1]oct-1-yl]methoxy}pentanoate (1.75 g, 3.00 mmol) in acetonitrile (5 mL) and the reaction was stirred under nitrogen at ambient temperature for 2 hours. The reaction was then diluted with 1 N hydrochloric acid (50 ml) and extracted with dichloromethane (2×125 mL). The combined dichloromethane extracts were dried over sodium sulfate, filtered and concentrated. The resultant residue was purified by column chromatography (20-100% ethyl acetate in heptane and then 0-30% methanol in dichloromethane) to afford the title compound as a white solid (1.71 g, 89%). ¹H NMR (600 MHz, CD₃OD) δ ppm 7.37-7.28 (m, 5H), 5.46 (d, 2H), 5.34 (s, 2H), 5.17-5.07 (m, 4H), 4.20 (d, 2H), 4.15-4.09 (m, 1H), 4.02 (d, 2H), 3.67-3.79 (m, 8H), 3.57-3.18 (m, 18H, overlapping with methanol peak), 2.45 (t, 4H), 2.38 (t, 2H), 2.25-2.12 (m, 12H), 1.96 (s, 12H), 1.76-1.48 (m, 14H), 1.43-1.25 (m, 14H).

### Step 10: Example 32

A solution of benzyl 1-[(1S,2R,3R,4R,5S)-4-(acetylamino)-2,3-bis(acetyloxy)-6,8-dioxabicyclo[3.2.1]oct-1-yl]-18-{17-[(1S,2R,3R,4R,5S)-4-(acetylamino)-2,3-bis(acetyloxy)-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oate (1.71 g, 1.18 mmol) in methanol (24 mL) was passed through a small 10% Pd/C Catcart on the H-cube under full H2 (20 bar) at 25° C. and 1 mL/min flow rate. The Catcart was rinsed with additional methanol (10 mL) and the flow-through was concentrated to an oil. The residue was dissolved in dichloromethane and concentrated (2×25 mL) to afford a sticky white foam. The material was dried on vacuum pump overnight and subsequently dissolved in acetonitrile/water (3.8 mL, 1:1) and lyophilized to a white solid (1.52 g, 95%). ¹H NMR (400 MHz, CD₃OD) δ ppm 5.44 (d, 2H), 5.32 (s, 2H), 5.12-5.09 (m, 2H), 4.18 (d, 2H), 4.13-4.10 (m, 1H) 4.01 (d, 2H) 3.79-3.67 (m, 8H), 3.56-3.35 (m, 10H), 3.26-3.14 (m, 8H), 2.43 (t, 4H), 2.28 (t, 2H), 2.23-2.14 (m, 12H) 1.97-1.92 (m, 12H), 1.74-1.50 (m, 14H), 1.41-1.28 (m, 14H). LCMS (m/z) for C₆₃H₁₀₄N₇O₂₅⁺ (M+H)⁺ 1359.1; retention time=0.80 min (UPLC 1.5 min run).

### 1-[(1S,2R,3R,4R,5S)-4-(acetylamino)-2,3-dihydroxy-6,8-dioxabicyclo[3.2.1]oct-1-yl]-18-{17-[(1S,2R,3R,4R,5S)-4-(acetylamino)-2,3-dihydroxy-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oic acid

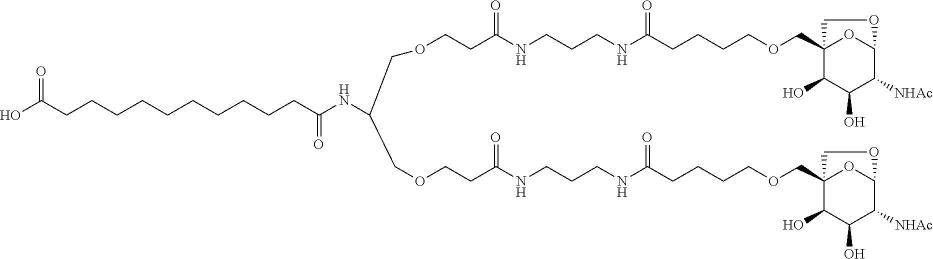 [SMILES: CC(=O)N[C@H]1[C@H]2OC[C@](COCCCCC(=O)NCCCNC(=O)CCOCC(COCCC(=O)NCCCNC(=O)CCCCOC[C@@]34CO[C@@H](O3)[C@H](NC(C)=O)[C@@H](O)[C@H]4O)NC(=O)CCCCCCCCCCC(=O)O)(O2)[C@H](O)[C@@H]1O]

### Reaction Scheme:

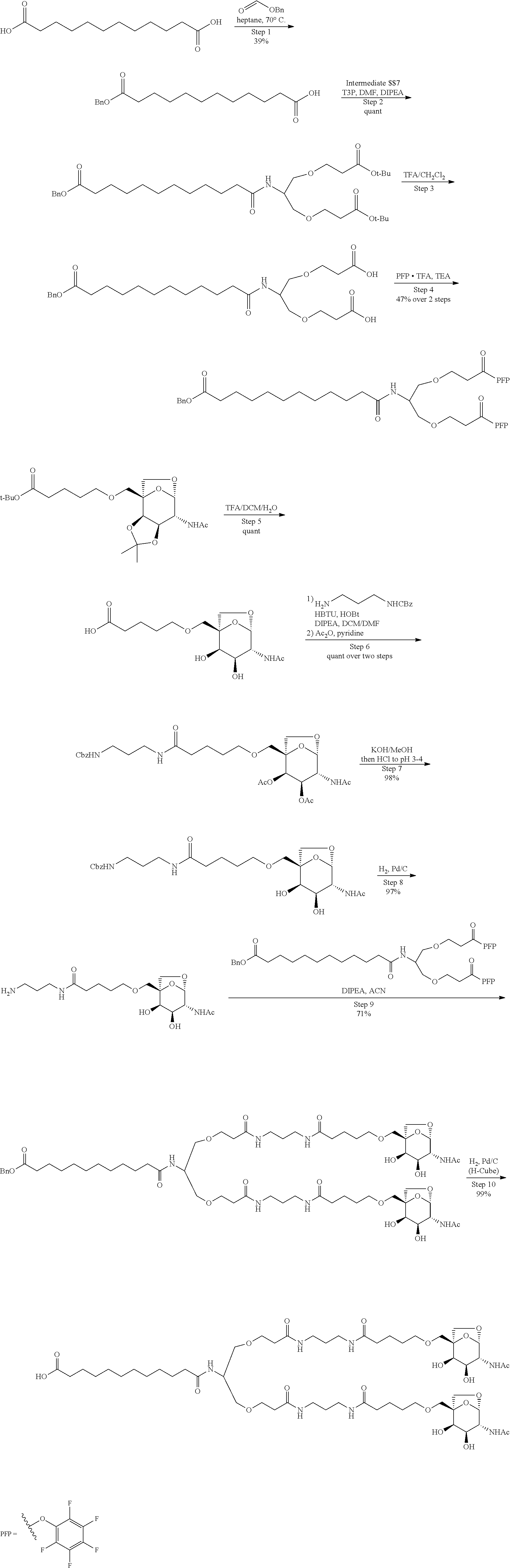

### Step 1: 12-(Benzyloxy)-12-oxododecanoic acid

Dodecanedioic acid (15.0 g, 65.0 mmol) and Dowex-H-form (65 g) were added to a mixture of heptane (0.52 L), and benzyl formate (142 g, 1.04 mol, 130 mL) and the reaction was heated to reflux for 24 h. The reaction was then cooled and the resin filtered off. The filtrate was concentrated in vacuo and the resultant residue was purified by silica gel chromatography (0-20% ethyl acetate in heptane) to afford a slushy residue. The isolate was slurried overnight (5% ethyl acetate in heptane, 20 mL). The mixture was filtered by vacuum filtration and the resultant solid washed with heptane to afford the tile compound as a white solid (6.66 g, 39%). ¹H NMR (400 MHz, CDCl₃) δ ppm 7.36 (s, 5H), 5.12 (s, 2H), 2.42-2.30 (m, 4H), 1.75-1.54 (m, 4H), 1.45-1.16 (m, 12H).

### Step 2: Benzyl 12-oxo-12-[(2,2,16,16-tetramethyl-4,14-dioxo-3,7,11,15-tetraoxaheptadecan-9-yl)amino]dodecanoate

n-Propylphosphonic acid anhydride, cyclic trimer (2.3 g, 3.6 mmol, 2.0 mL, 50% in ethyl acetate) was added to a suspension of 12-(benzyloxy)-12-oxododecanoic acid (0.95 g, 2.98 mmol), di-tert-butyl 3,3′-[(2-aminopropane-1,3-diyl)bis(oxy)]dipropanoate (1.03 g, 2.98 mmol) and N,N-diisopropylethylamine (1.2 g, 8.9 mmol, 1.6 mL) in dimethylformamide (8 mL) and the reaction mixture was stirred at room temperature overnight. The reaction mixture was diluted with ethyl acetate (100 mL) and washed with hydrochloric acid (1 N, 30 mL), saturated sodium bicarbonate (30 mL), and brine (30 mL). The organic phase was dried over sodium sulfate, filtered, and concentrated to afford a residue which was then azeotroped with heptane (3×40 mL) to afford the title compound as a solid (2.0 g, 100%). ¹H NMR (600 MHz, CD₃CN) δ ppm 7.41-7.31 (m, 5H), 6.34 (d, 1H), 5.08 (s, 2H), 4.01 (td, 1H), 3.67-3.58 (m, 4H), 3.44 (dd, 2H), 3.36 (dd, 2H), 2.40 (t, 4H), 2.33 (t, 2H), 2.09 (t, 2H), 1.61-1.49 (m, 4H), 1.43 (s, 18H), 1.30-1.25 (m, 12H). LCMS (m/z) for C₃₆H₆₀NO₉⁺ (M+H)⁺650.5; retention time=1.23 min (UPLC 1.5 min method)

### Step 3: 3,3′-[(2-{[12-(benzyloxy)-12-oxododecanoyl]amino}propane-1,3-diyl)bis(oxy)]dipropanoic acid

A solution of 12-oxo-12-[(2,2,16,16-tetramethyl-4,14-dioxo-3,7,11,15-tetraoxaheptadecan-9-yl)amino]dodecanoate (1.60 g, 2.47 mmol) in trifluoracetic acid (15 g, 130 mmol, 10.0 mL) was stirred at ambient temperature overnight. The reaction mixture was concentrated and the resultant residue was azeotroped with diethyl ether (4×70 mL) at 20° C. and subsequently dried in vacuo overnight to afford the title compound as a gum (4.03 g, 99%, 2.11 equiv TFA). ¹H NMR (600 MHz, CD₃CN) δ ppm 7.52-7.24 (m, 5H), 6.89 (d, 1H), 5.14-5.04 (m, 2H), 4.15-4.01 (m, 1H), 3.65 (t, 4H), 3.49-3.46 (m, 2H), 3.44-3.41 (m, 2H), 2.50 (t, 4H), 2.33 (t, 2H), 2.21 (t, 2H), 1.57 (td, 4H), 1.23-1.32 (m, 12H). LCMS (m/z) for C₂₈H₄₄NO₉⁺ (M+H)⁺ 538.6; retention time=0.92 min (UPLC 1.5 min method).

### Step 4: Benzyl 12-({1,3-bis[3-oxo-3-(pentafluorophenoxy)propoxy]propan-2-yl}amino)-12-oxododecanoate

To a suspension of of 3,3′-[(2-{[12-(benzyloxy)-12-oxododecanoyl]amino}propane-1,3-diyl)bis(oxy)]dipropanoic acid (1.57 g, 2.41 mmol, 1 equiv TFA) di-isopropylethylamine (3.11 g, 24.1 mmol, 4.20 mL) in dimethylformamide (10 mL) pentafluorophenyl trifluoroacetate (5.40 g, 19.3 mmol, 3.31 mL) was added dropwise in an ice-bath. The resulting solution was stirred at ambient temperature. The reaction mixture was concentrated and azeotroped with heptane (2×20 mL). The resulting residue was diluted with ethyl acetate (80 mL), washed with 10% citric acid (30 mL), saturated sodium bicarbonate (30 mL), and brine (30 mL), dried over Na₂SO₄, and concentrated. The crude was purified by silica gel chromatography (0-60% ethyl acetate in heptane) to afford the title compound as a solid (983 mg, 47%). ¹H NMR (600 MHz, CD₃CN-d₃) δ ppm 7.44-7.24 (m, 5H), 6.23 (d, 1H), 5.08 (s, 2H), 4.08 (td, 1H), 3.81-3.76 (m, 4H), 3.53-3.42 (m, 4H), 2.92 (t, 4H), 2.32 (t, 2H), 2.07 (t, 2H), 1.62-1.46 (m, 4H), 1.32-1.19 (m, 12H). LCMS (m/z) for C₄₀H₄₂NO₉⁺ (M+H)⁺ 870.6; retention time=1.23 min (UPLC 1.5 min method).

### Step 5: 5-{[(1S,2R,3R,4R,5S)-4-(Acetylamino)-2,3-dihydroxy-6,8-dioxabicyclo[3.2.1]oct-1-yl]methoxy}pentanoic acid

To a solution of tert-butyl 5-{[(1S,2R,6R,7R,8S)-7-(acetylamino)-4,4-dimethyl-3,5,9,11-tetraoxatricyclo[6.2.1.0˜2,6˜]undec-1-yl]methoxy}pentanoate (2.09 g, 4.87 mmol) in dichloromethane (15 mL) and water (2 mL) was added trifluoroacetic acid (22 g, 200 mmol, 15 mL) and the reaction mixture was stirred at ambient temperature overnight. The crude was concentrated, azeotroped with toluene (3×50 mL), then heptane (3×50 ml), and dried in vacuo to afford the title compound as a gum (2.08 g, quantitative, 0.83 equiv TFA). LCMS (m/z) for C₁₄H₂₄NO₈⁺ (M+H)⁺ 334.2; retention time=0.45 min (UPLC 1.5 min method)

### Step 6: (1S,2R,3R,4R,5S)-4-(Acetylamino)-1-(3,9-dioxo-1-phenyl-2,14-dioxa-4,8-diazapentadecan-15-yl)-6,8-dioxabicyclo[3.2.1]octane-2,3-diyl diacetate

To solution of 5-{[(1S,2R,3R,4R,5S)-4-(acetylamino)-2,3-dihydroxy-6,8-dioxabicyclo[3.2.1]oct-1-yl]methoxy}pentanoic acid containing about 1 equiv TFA (1.0 g, 2.05 mmol) in dichloromethane (12 mL) and dimethylformamide (5 mL) was added di-isopropylethylamine (1.59 g, 12.3 mmol), o-(benzotriazol-1-yl)-N,N,N′,N′-tetramethyluronium hexafluorophosphate (0.93 g, 2.46 mmol) and 1H-benzotriazol-1-ol (277 mg, 2.05 mmol). The cloudy mixture was stirred at room temperature for 20 min. Benzyl n-(3-aminopropyl)carbamate hydrochloride (502 mg, 2 mmol) was then added. The resulting mixture was stirred at ambient temperature overnight. Formation of amide product was confirmed by LCMS [C₂₅H₃₈N₃O₉⁺, (M+H)⁺ 524.5]. The reaction mixture was concentrated to 5 mL and diluted with pyridine (6 mL). To this solution was added acetic anhydride (6.0 g, 60 mmol) and the reaction mixture was stirred at 50° C. overnight. The reaction mixture was cooled to room temperature, diluted with ethyl acetate (70 mL), washed with hydrochloric acid (1 N, 30 mL), saturated sodium bicarbonate (30 mL), and brine (30 mL). The separated organic phase was dried over sodium sulfate, filtered, and concentrated. The crude was purified by silica gel chromatography (0-15% methanol in dichloromethane) to afford the title compound as a glass (1.28 g, 100%). LCMS (m/z) for C₂₉H₄₂N₃O₁₁⁺, (M+H)⁺ 608.5; retention time=0.70 min (UPLC 1.5 min method)

### Step 7: Benzyl {3-[(5-{[(1S,2R,3R,4R,5S)-4-(acetylamino)-2,3-dihydroxy-6,8-dioxabicyclo[3.2.1]oct-1-yl]methoxy}pentanoyl)amino]propyl}carbamate

To a suspension (1S,2R,3R,4R,5 S)-4-(acetylamino)-1-(3,9-dioxo-1-phenyl-2,14-dioxa-4,8-diazapentadecan-15-yl)-6,8-dioxabicyclo[3.2.1]octane-2,3-diyl diacetate (1.5 g, 2.47 mmol) in methanol (8 mL) was added potassium hydroxide (1 M in methanol, 5.3 mL, 5.3 mmol). The reaction mixture was stirred at room temperature for 1 h. The reaction mixture was then treated with hydrochloric acid (4.0 M in dioxane, 1.5 mL) dropwise. The resulting slurry was concentrated and triturated in ethanol (15 mL) for 10 min. The resulting potassium chloride precipitate was removed by filtration, rinsed by ethanol (5 mL). The filtrate was concentrated, dried by vacuum to afford the entitled compound as a gum (1.28 g, 98%). ¹H NMR (600 MHz, MATHANOL-d₄) δ ppm 7.41-7.25 (m, 5H), 5.21 (s, 1H), 5.07 (s, 2H), 3.93 (dd, 2H), 3.86 (d, 1H), 3.77 (d, 1H), 3.71 (dd, 1H), 3.65 (d, 1H), 3.58 (d, 1H), 3.53-3.46 (m, 2H), 3.20 (t, 2H), 3.15 (t, 2H), 2.20 (t, 2H), 1.99 (s, 3H), 1.70-1.62 (m, 4H), 1.61-1.53 (m, 2H). LCMS (m/z) for C₂₅H₃₈N₃O₉⁺, (M+H)⁺ 524.5; retention time=0.64 min (UPLC 1.5 min method)

### Step 8: 5-{[(1S,2R,3R,4R,5S)-4-(Acetylamino)-2,3-dihydroxy-6,8-dioxabicyclo[3.2.1]oct-1-yl]methoxy}-N-(3-aminopropyl)pentanamide

A mixture of benzyl {3-[(5-{[(1S,2R,3R,4R,5S)-4-(acetylamino)-2,3-dihydroxy-6,8-dioxabicyclo[3.2.1]oct-1-yl]methoxy}pentanoyl)amino]propyl}carbamate (1.60 g, 2.4 mmol) and 10% palladium on carbon (200 mg) in methanol (20 mL) was stirred under hydrogen pressure (50 psi) at ambient temperature in a Parr reactor overnight. The reaction mixture was filtered through celite. The celite was washed with methanol (50 mL) and the combined filtrates were concentrated, and dried in vacuo to afford the title compound as a solid (925 mg, 97%). ¹H NMR (600 MHz, MATHANOL-d₄) δ ppm 5.21 (s, 1H), 3.94 (d, 1H), 3.90 (d, 1H), 3.86 (d, 1H), 3.77 (d, 1H), 3.71 (dd, 1H), 3.65 (d, 1H), 3.61 (d, 1H), 3.58-3.46 (m, 2H), 3.26 (t, 2H), 2.82 (t, 2H), 2.22 (t, 2H), 1.99 (s, 3H), 1.76 (quin, 2H), 1.68 (quin, 2H), 1.63-1.56 (m, 2H). LCMS (m/z) for C₁₇H₃₂N₃O₇⁺, (M+H)⁺ 390.5; retention time=0.47 min (UPLC 1.5 min method)

### Step 9: Benzyl 1-[(1S,2R,3R,4R,5S)-4-(acetylamino)-2,3-dihydroxy-6,8-dioxabicyclo[3.2.1]oct-1-yl]-18-{17-[(1S,2R,3R,4R,5S)-4-(acetylamino)-2,3-dihydroxy-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oate

A mixture of 5-{[(1S,2R,3R,4R,5S)-4-(acetylamino)-2,3-dihydroxy-6,8-dioxabicyclo[3.2.1]oct-1-yl]methoxy}-N-(3-aminopropyl)pentanamide (607 mg, 1.40 mmol) and benzyl 12-({1,3-bis[3-oxo-3-(pentafluorophenoxy)propoxy]propan-2-yl}amino)-12-oxododecanoate (500 mg, 0.58 mmol), and N,N-diisopropylethylamine (200 mg, 2.0 mmol) in a mixture of dichloromethane (8 mL) and dimethylformamide (3 mL) was stirred at room temperature overnight. The reaction mixture was concentrated, azeotroped with heptane (3×10 mL), and concentrated. The crude residue was purified by silica gel chromatography (0-40% methanol in dichloromethane) to afford the title compound as a glass (520 mg, 71%). ¹H NMR (600 MHz, METHANOL-d₄) δ ppm 7.37-7.29 (m, 5H), 5.21 (s, 2H), 5.11 (s, 2H), 4.11 (t, 1H), 3.94 (dd, 4H), 3.87 (d, 2H), 3.77 (d, 2H), 3.75-3.67 (m, 6H), 3.64 (d, 2H), 3.57 (d, 2H), 3.55-3.42 (m, 8H), 3.23-3.19 (m, 8H), 2.43 (t, 4H), 2.36 (t, 2H), 2.20 (q, 6H), 1.98 (s, 6H), 1.72-1.54 (m, 16H), 1.35-1.28 (m, 12H). LCMS C₆₂H₁₀₂N₇O₂₁⁺, (M+H)⁺ 1280.3; retention time=1.45 min (UPLC 3 min method)

### Step 10: Example 33. 1-[(1S,2R,3R,4R,5S)-4-(acetylamino)-2,3-dihydroxy-6,8-dioxabicyclo[3.2.1]oct-1-yl]-18,18-bis{17-[(1S,2R,3R,4R,5S)-4-(acetylamino)-2,3-dihydroxy-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oic acid

A solution of benzyl 1-[(1S,2R,3R,4R,5S)-4-(acetylamino)-2,3-dihydroxy-6,8-dioxabicyclo[3.2.1]oct-1-yl]-18-{17-[(1S,2R,3R,4R,5S)-4-(acetylamino)-2,3-dihydroxy-6,8-dioxabicyclo[3.2.1]oct-1-yl]-5,11-dioxo-2,16-dioxa-6,10-diazaheptadec-1-yl}-7,13,20-trioxo-2,16-dioxa-8,12,19-triazahentriacontan-31-oate (520 mg, 0.41 mmol) in methanol (16 mL) was passed through 10% Pd/C 30×4 CatCart® on ThalesNano H-Cube Pro™ with a flow rate of 1 mL/min at 25° C. under full Hz. The system was rinsed by methanol (20 mL). The filtrate was concentrated, azeotroped with methylene chloride (20 mL), then heptane (20 mL). The resulting residue was dissolved in acetonitrile/water (1:1, 20 mL) and freeze dried to afford the title compound as a white solid (477 mg, 99%). ¹H NMR (600 MHz, METHANOL-d₄) δ ppm 5.21 (s, 2H), 4.11 (t, 1H), 3.98-3.87 (m, 4H), 3.87 (d, 2H), 3.78 (d, 2H), 3.74-3.67 (m, 6H), 3.65 (d, 2H), 3.58 (d, 2H), 3.55-3.45 (m, 8H), 3.21 (q, 8H), 2.43 (t, 4H), 2.27 (t, 2H), 2.23-2.16 (m, 6H), 1.99 (s, 6H), 1.72-1.62 (m, 8H), 1.63-1.54 (m, 8H), 1.35-1.28 (m, 12H). LCMS (m/z) for C₅₅H₉₆N₇O₂₁⁺, (M+H)⁺ 1190.7; retention time=1.07 min (UPLC 3 min method).

### 1-[(1S,2R,3R,4R,5S)-4-(acetylamino)-2,3-dihydroxy-6,8-dioxabicyclo[3.2.1]oct-1-yl]-7,13,20-trioxo-2,16-dioxa-8,12,19-triazahentriacontan-31-oic acid

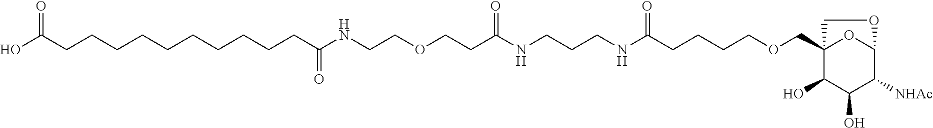 [SMILES: CC(=O)N[C@H]1[C@H]2OC[C@](COCCCCC(=O)NCCCNC(=O)CCOCCNC(=O)CCCCCCCCCCC(=O)O)(O2)[C@H](O)[C@@H]1O]

### Reaction Scheme:

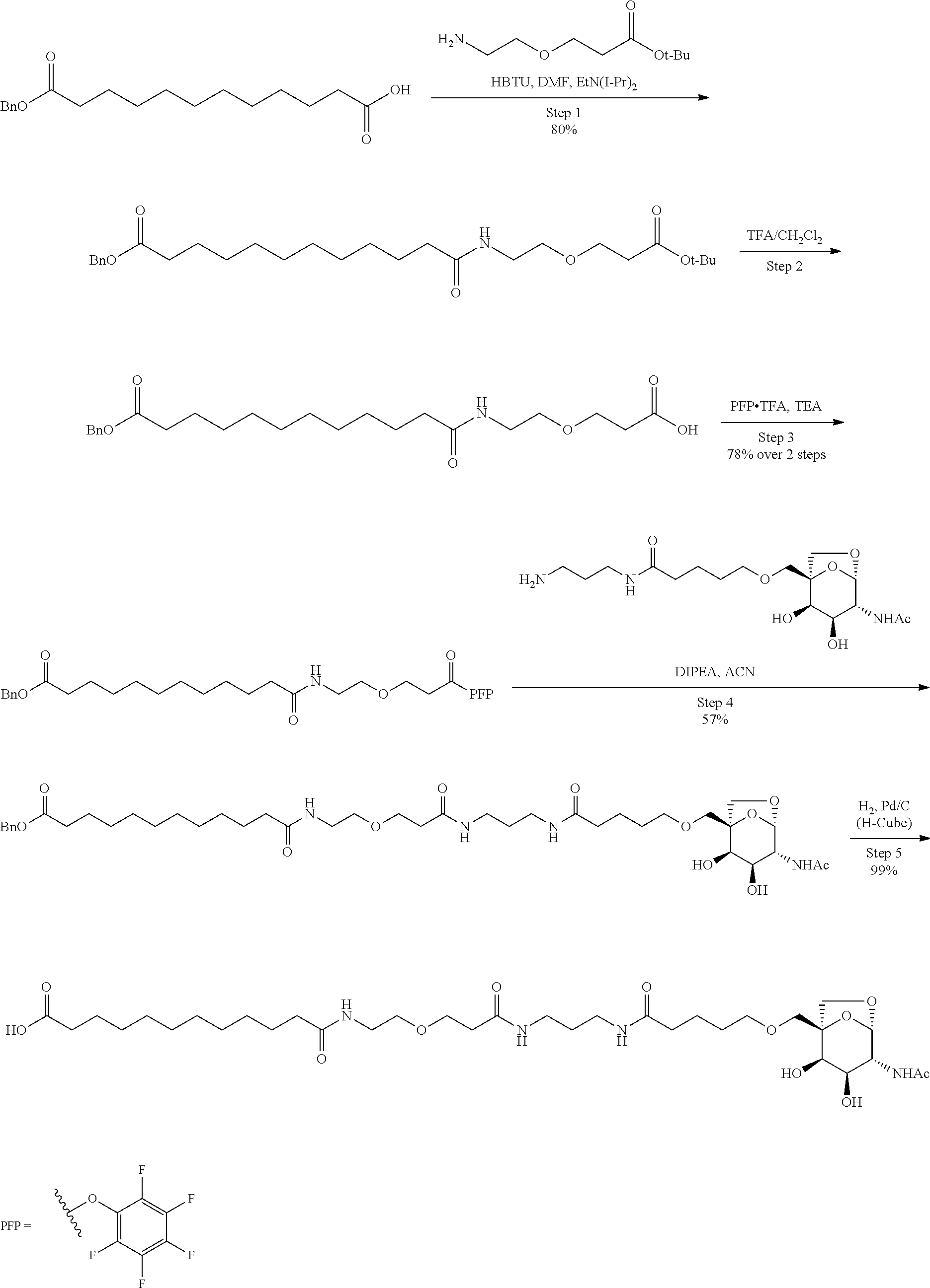

### Step 1: Benzyl 12-{[2-(3-tert-butoxy-3-oxopropoxy)ethyl]amino}-12-oxododecanoate

12-benzyloxy-12-oxodececanoic acid (2.33 g, 7.26 mmol), tert-butyl 3-(2-aminoethoxy)propanoate (1.25 g, 6.60 mmol) and N,N-diisopropylethylamine (2.3 ml, 13 mmol) were dissolved in N,N-dimethylformamide (35 mL). To this solution was added N,N,N′,N′-tetramethyl-O-(1H-benzotriazol-1-yl)uronium hexafluorophosphate, O-(Benzotriazol-1-yl)-N,N,N′,N′-tetramethyluronium hexafluorophosphate (2.75 g, 7.26 mmol) and the reaction stirred at ambient temperature for 16 h. The reaction was concentrated and the residue was dissolved in ethyl acetate (100 mL) and washed sequentially with saturated sodium bicarbonate, water, and brine (25 mL each). The organic layer was then dried over sodium sulfate, filtered and concentrated to a colorless oil. The residue was purified by silica gel chromatography (0-100% ethyl acetate in heptane) to afford the desired product as a white solid (2.58 g, 80%). ¹H NMR (400 MHz, CDCl₃) δ ppm 7.41-7.30 (m, 5H), 6.18 (br.s., 1H), 5.12 (s, 2H), 3.69 (t, 2H), 3.57-3.51 (m, 2H), 3.49-3.42 (m, 2H), 2.49 (t, 2H), 2.36 (t, 2H), 2.23-2.14 (m, 2H), 1.63 (d, 4H), 1.47 (s, 9H), 1.36-1.22 (m, 12H).

### Step 2: 3-(2-{[12-(Benzyloxy)-12-oxododecanoyl]amino}ethoxy)propanoic acid

Benzyl 12-{[2-(3-tert-butoxy-3-oxopropoxy)ethyl]amino}-12-oxododecanoate (2.58 g, 5.25 mmol) was dissolved in dichloromethane (12 mL). To this was added trifluoroacetic acid (20 ml, 0.27 mol). After 2 h stirring at ambient temperature, the reaction was concentrated. The resultant residue was dissolved in toluene and concentrated (2×20 mL) and subsequently dried on vacuum pump to afford a solid (2.21 g). ¹H NMR (400 MHz, CDCl₃) δ ppm 7.41-7.30 (m, 5H) 6.08 (br.s., 1H) 5.12 (s, 2H) 3.74 (t, 2H) 3.61-3.51 (m, 2H) 3.50-3.40 (m, 2H) 2.64 (t, 2H) 2.36 (t, 2H) 2.25-2.11 (m, 2H) 1.72-1.54 (m, 4H) 1.27 (m, 12H).

### Step 3: Benzyl 12-oxo-12-({2-[3-oxo-3-(pentafluorophenoxy)propoxy]ethyl}amino)dodecanoate

N,N-diisopropylethylamine (3.52 ml, 20.2 mmol) was added to a solution of 3-(2-{[12-(benzyloxy)-12-oxododecanoyl]amino}ethoxy)propanoic acid (2.20 g, 5.05 mmol) in dimethylformamide (24 mL). To this was then added pentafluorophenol-2,2,2-trifluoroacetate (1.74 mL, 10.1 mmol) in a slow stream. The reaction turned purple in color and was stirred at ambient temperature for 18 h. The reaction mixture was concentrated to ⅓ volume on a rotary evaporator (50° C., high vacuum pump) and the resultant concentrate was diluted with ethyl acetate (300 mL), washed with 10% citric acid (100 mL), saturated sodium bicarbonate (100 mL) and brine (100 mL). The organic layer was dried over sodium sulfate, filtered, and concentrated. The residue was purified by silica gel chromatography (0-100% ethyl acetate in heptane) to afford the desired product as a yellow solid (2.46 g, 78% over 2 steps). ¹H NMR (400 MHz, CDCl₃) δ ppm 7.41-7.30 (m, 5H), 5.85 (br.s., 1H) 5.12 (s, 2H), 3.85 (t, 2H), 3.64-3.54 (m, 2H), 3.52-3.43 (m, 2H), 2.94 (t, 2H), 2.36 (t, 2H), 2.16 (t, 2H), 1.70-1.60 (m, 4H), 1.26 (m, 12H).

### Step 4: Benzyl 1-[(1S,2R,3R,4R,5S)-4-(acetylamino)-2,3-dihydroxy-6,8-dioxabicyclo[3.2.1]oct-1-yl]-20-oxo-2,16-dioxa-8,12,19-triazahentriacontan-31-oate

A mixture of 5-{[(1S,2R,3R,4R,5S)-4-(acetylamino)-2,3-dihydroxy-6,8-dioxabicyclo[3.2.1]oct-1-yl]methoxy}-N-(3-aminopropyl)pentanamide (172 mg, 0.440 mmol) and benzyl 12-oxo-12-({2-[3-oxo-3-(pentafluorophenoxy)propoxy]ethyl}amino)dodecanoate (220 mg, 0.402 mmol), N,N-diisopropylethylamine (95 mg, 0.73 mmol) in a mixture of dichloromethane (3.3 mL) and dimethylformamide (0.7 mL) was stirred at room temperature overnight. The reaction mixture was concentrated, azeotroped with heptane (3×10 mL), and concentrated. The crude was purified by silica gel chromatography (0-25% methanol in dichloromethane to afford the title compound as an oil (185 mg, 57%). ¹H NMR (600 MHz, METHANOL-d₄) δ ppm 7.39-7.28 (m, 5H), 5.21 (s, 1H), 5.11 (s, 2H), 3.99-3.89 (m, 2H), 3.87 (d, 1H), 3.77 (d, 1H), 3.75-3.67 (m, 3H), 3.64 (d, 1H), 3.57 (d, 1H), 3.55-3.44 (m, 4H), 3.38-3.32 (m, 2H), 3.26-3.16 (m, 4H), 2.44 (t, 2H), 2.36 (t, 2H), 2.19 (td, 4H), 1.99 (s, 3H), 1.70-1.55 (m, 10H), 1.33-1.25 (m, 12H). LCMS (m/z) for C₄₁H₆₇N₄O₁₂⁺, (M+H)⁺ 807.8; retention time=1.60 min (UPLC 3 min method).

### Step 5: Example 34

A solution of benzyl 1-[(1S,2R,3R,4R,5S)-4-(acetylamino)-2,3-dihydroxy-6,8-dioxabicyclo[3.2.1]oct-1-yl]-20-oxo-2,16-dioxa-8,12,19-triazahentriacontan-31-oate (0.22 g, 0.25 mmol) in methanol (22 mL) was passed through 10% Pd/C 30×4 CatCart® on ThalesNano H-cube Pro™ with a flow rate of 1 mL/min at 25° C. under full H2. The system was rinsed by methanol (40 mL). The filtrate was concentrated, azeotroped with methylene chloride (20 mL), then heptane (20 mL). The resulting residue was dissolved in acetonitrile/water (1:1, 20 mL) and freeze dried to afford the title compound as a white solid (177 mg, 99%). ¹H NMR (600 MHz, METHANOL-d₄) δ ppm 5.20 (s, 1H), 4.01-3.89 (m, 2H), 3.87 (d, 1H), 3.77 (d, 1H), 3.75-3.68 (m, 3H), 3.65 (d, 1H), 3.57 (d, 1H), 3.55-3.45 (m, 4H), 3.34 (t, 2H), 3.27-3.14 (m, 4H), 2.44 (t, 2H), 2.28 (t, 2H), 2.20 (td, 4H), 1.98 (s, 3H), 1.67 (t, 4H), 1.63-1.54 (m, 6H), 1.35-1.28 (m, 12H). C₃₄H₆₁N₄O₁₂⁺, (M+H)⁺ 717.7; retention time=1.12 min (UPCL 3 min method).

### 5,11,18-trioxo-16-{[3-oxo-3-({3-[(5-{[3,4,6-tri-O-acetyl-2-(acetylamino)-2-deoxy-β-D-galactopyranosyl]oxy}pentanoyl)amino]propyl}amino)propoxy]methyl}-1-{[3,4,6-tri-O-acetyl-2-(acetylamino)-2-deoxy-β-D-galactopyranosyl]oxy}-14-oxa-6,10,17-triazanonacosan-29-oic acid

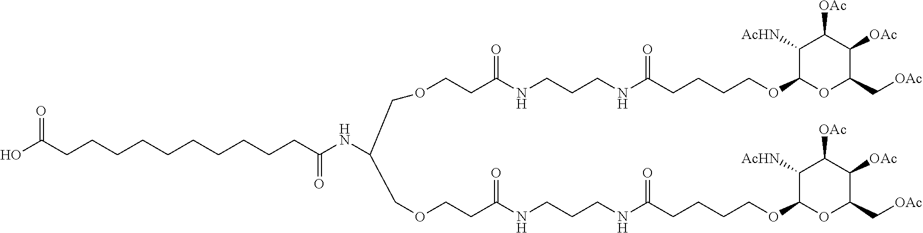 [SMILES: CC(=O)N[C@H]1[C@H](OCCCCC(=O)NCCCNC(=O)CCOCC(COCCC(=O)NCCCNC(=O)CCCCO[C@@H]2O[C@H](COC(C)=O)[C@H](OC(C)=O)[C@H](OC(C)=O)[C@H]2NC(C)=O)NC(=O)CCCCCCCCCCC(=O)O)O[C@H](COC(C)=O)[C@H](OC(C)=O)[C@@H]1OC(C)=O]

### Reaction Scheme:

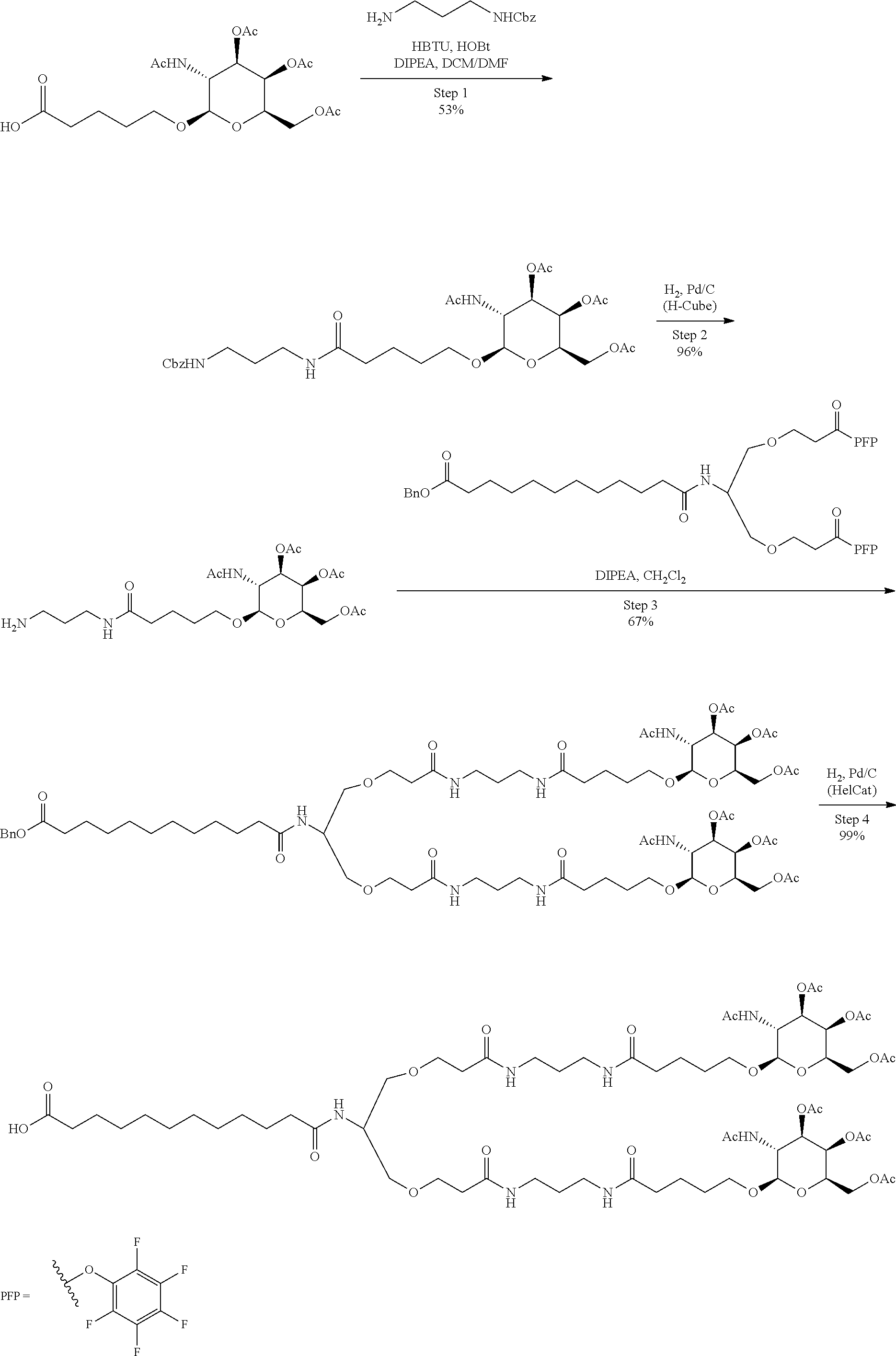

### Step 1: Benzyl {3-[(5-{[3,4,6-tri-O-acetyl-2-(acetylamino)-2-deoxy-β-D-galactopyranosyl]oxy}pentanoyl)amino]propyl}carbamate

N,N-diisopropylethylamine (1.38 mL, 7.95 mmol) was added to a solution of benzyl (3-aminopropyl)carbamate hydrochloride salt (0.713 g, 2.01 mmol) in N,N-dimethylformamide (3.5 mL). The resultant mixture was then added to a solution of 5-(((2R,3R,4R,5R,6R)-3-acetamido-4,5-diacetoxy-6-(acetoxymethyl)tetrahydro-2H-pyran-2-yl)oxy)pentanoic acid (1.19 g, 2.65 mmol) in dimethylformamide (10 mL). 1-[Bis(dimethylamino)methylene]-1H-1,2,3-triazol[4,5-b]pyridinium 3-oxid hexafluorophosphate (1.11 g, 2.91 mmol) was then added and the reaction mixture was stirred at ambient temperature under an atmosphere of nitrogen (2.5 h). The reaction was quenched with saturated ammonium chloride (50 mL) and extracted with dichloromethane (4×75 mL). The combined organic layers were washed with brine (50 mL), dried over sodium sulfate, filtered and concentrated. The resultant oil was purified by silica gel chromatography (2-6% methanol in dichloromethane) to afford the desired product as a white foam (0.904 g, 53%). ¹H NMR (400 MHz, CD₃OD) δ ppm 7.41-7.25 (m, 5H), 5.33 (d, 1H), 5.12-5.02 (m, 3H), 4.54 (d, 1H), 4.19-4.03 (m, 3H), 4.03-3.97 (m, 1H), 3.91-3.82 (m, 1H), 3.60-3.46 (m, 2H), 3.11-3.25 (m, 4H), 2.16-2.23 (m, 2H), 2.13 (s, 3H), 2.02 (s, 3H), 1.95 (s, 3H), 1.92 (s, 3H), 1.73-1.53 (m, 6H), 1.40-1.34 (m, 2H).

### Step 2: N-(3-aminopropyl)-5-{[3,4,6-tri-O-acetyl-2-(acetylamino)-2-deoxy-β-D-galactopyranosyl]oxy}pentanamide acetate salt

Benzyl {3-[(5-{[3,4,6-tri-O-acetyl-2-(acetylamino)-2-deoxy-β-D-galactopyranosyl]oxy}pentanoyl)amino]propyl}carbamate 5-{[3,4,6-tri-O-acetyl-2-(acetylamino)-2-deoxy-β-D-galactopyranosyl]oxy}pentanoic acid (1.01 g, 1.59 mmol) was dissolved in a mixture of methanol (30 mL) and glacial acetic acid (91 uL, 1.6 mmol). To this was added 10% Pd/C (0.2 g, wet) under nitrogen and the reaction mixture was placed in a sealed, stirred Parr reactor under 50 psi hydrogen. After 16 h the head space was purged with nitrogen (×4) and filtered through a 0.45 um nylon syringe filter. The filter was washed with methanol and the combined filtrates were concentrated to afford the title compound as a white foam (0.86 g mg, 96%). ¹H NMR (400 MHz, CD₃OD) δ ppm 5.34 (d, 1H), 5.06-5.02 (m, 1H), 4.52 (d, 1H), 4.19-4.05 (m, 3H), 4.04-3.98 (m, 1H), 3.92-3.84 (m, 1H), 3.56-3.47 (m, 1H), 2.98-2.89 (m, 2H), 2.23 (t, 2H), 2.14 (s, 3H), 2.06 (d, 1H), 2.03 (s, 3H), 1.94 (d, 5H), 1.91 (s, 3H), 1.83 (m, 2H), 1.74-1.54 (m, 4H), 1.39-1.31 (m, 1H).

### Step 3: Benzyl 5,11,18-trioxo-16-{[3-oxo-3-({3-[(5-{[3,4,6-tri-O-acetyl-2-(acetylamino)-2-deoxy-β-D-galactopyranosyl]oxy}pentanoyl)amino]propyl}amino)propoxy]methyl}-1-{[3,4,6-tri-O-acetyl-2-(acetylamino)-2-deoxy-β-D-galactopyranosyl]oxy}-14-oxa-6,10,17-triazanonacosan-29-oate

Benzyl 12-({1,3-bis[3-oxo-3-(pentafluorophenoxy)propoxy]propan-2-yl}amino)-12-oxododecanoate (150 mg, 0.172 mmol), N-(3-aminopropyl)-5-{[3,4,6-tri-O-acetyl-2-(acetylamino)-2-deoxy-β-D-galactopyranosyl]oxy}pentanamide acetate salt (233 mg, 0.414 mmol) and N,N-diisopropylethylamine (0.15 ml, 0.862 mmol) were dissolved in dichloromethane (3.5 mL) and stirred at ambient temperature for 64 h. The reaction mixture was concentrated and heptane was added and the residue concentrated once again (×3). The resultant residue was purified by silica gel chromatography (4-14% methanol in dichloromethane) to afford the desired product as a colorless glass (175 mg, 67%). The compound was then dissolved in a mixture of acetonitrile and water (1:1, 30 mL) and freeze dried to afford a white solid. ¹H NMR (400 MHz, CDCl₃) δ ppm 7.42-7.37 (m, 5H), 7.15 (br.s., 2H) 6.94-6.84 (m, 3H), 6.51-6.45 (m, 2H), 5.38 (d, 2H), 5.20 (d, 2H), 5.14 (s, 2H), 4.65-4.57 (m, 2H), 4.27-4.06 (m, 8H), 4.02-3.90 (m, 4H), 3.73 (br.s., 6H), 3.63-3.42 (m, 8H), 3.37-3.26 (m, 8H), 2.47 (br.s., 5H), 2.38 (t, 3H), 2.34-2.15 (m, 15H), 2.12 (s, 6H), 2.03 (s, 6H), 1.98 (s, 6H), 1.78 (br.s., 4H) 1.28 (d, 14H).

### Step 4: Example 35

10% Pd/C (30 mg, wet) was added to a solution of Benzyl-5,11,18-trioxo-16-{[3-oxo-3-({3-[(5-{[3,4,6-tri-O-acetyl-2-(acetylamino)-2-deoxy-β-D-galactopyranosyl]oxy}pentanoyl)amino]propyl}amino)propoxy]methyl}-1-{[3,4,6-tri-O-acetyl-2-(acetylamino)-2-deoxy-β-D-galactopyranosyl]oxy}-14-oxa-6,10,17-triazanonacosan-29-oate (282 mg, 0.187 mmol) in methanol (5 mL). The mixture was placed in the HelCat under 50 psi hydrogen and stirred at ambient temperature for 16 h. The head space was then purged with nitrogen (×3) and the solution was filtered through a 0.2 um nylon syringe filter. The filter was washed with methanol and the combined filtrates were concentrated to afford the title compound as a white foam which was then dissolved in a mixture of acetonitrile and water (1:1, 20 mL) and freeze dried to a white solid (262 mg, 99%). ¹H NMR (400 MHz, CD₃OD) δ ppm 5.33 (d, 2H), 5.08-5.04 (m, 2H), 4.55 (d, 2H) 4.21-3.98 (m, 10H), 3.91-3.83 (m, 2H), 3.69 (t, 4H) 3.60-3.44 (m, 8H), 3.24-3.19 (m, 8H), 2.43 (t, 4H), 2.31-2.16 (m, 8H), 2.14 (s, 6H), 2.06-2.00 (m, 6H), 1.94 (d, 10H), 1.73-1.55 (m, 15H), 1.40-1.28 (m, 12H). LCMS (m/z) for C₆₅H₁₀₈N₇O₂₇⁺ (M+H)⁺ 1419.7; retention time=1.34 min (UPLC 3.0 min run)

### 5,11,18-trioxo-1-{[3,4,6-tri-O-acetyl-2-(acetylamino)-2-deoxy-β-D-galactopyranosyl]oxy}-14-oxa-6,10,17-triazanonacosan-29-oic acid

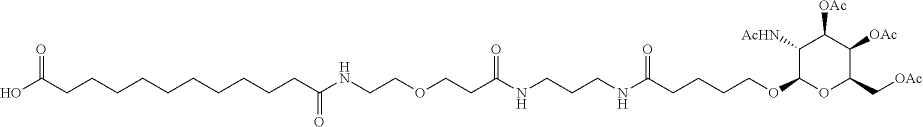

### Reaction Scheme:

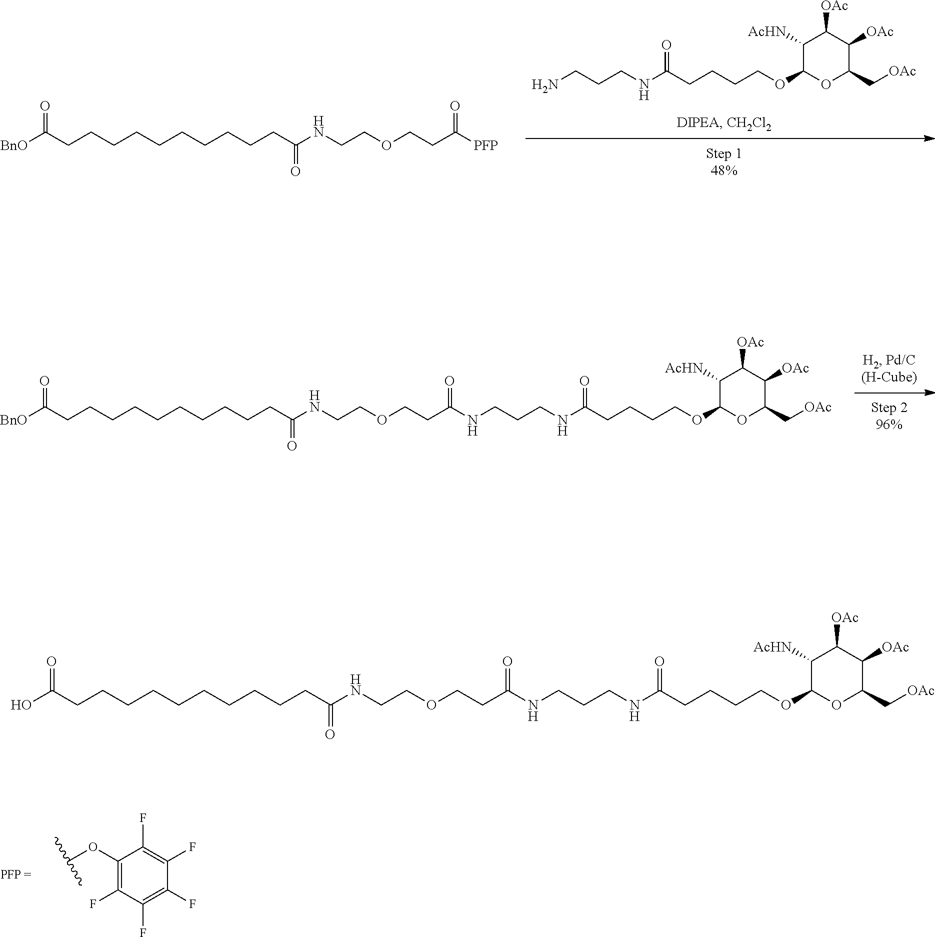

### Step 1: Benzyl 5,11,18-trioxo-1-{[3,4,6-tri-O-acetyl-2-(acetylamino)-2-deoxy-β-D-galactopyranosyl]oxy}-14-oxa-6,10,17-triazanonacosan-29-oate

N-(3-aminopropyl)-5-{[3,4,6-tri-O-acetyl-2-(acetylamino)-2-deoxy-β-D-galactopyranosyl]oxy}pentanamide acetate salt (159 mg, 0.264 mmol) was added to a solution of benzyl 12-oxo-12-({2-[3-oxo-3-(pentafluorophenoxy)propoxy]ethyl}amino)dodecanoate (164 mg, 0.291 mmol) and N,N-diisopropylethylamine (200 uL, 1.32 mmol) in dichloromethane (5 mL) and the reaction was stirred at ambient temperature for 16 h. The reaction mixture was concentrated and the resultant residue was taken up in heptane and concentrated (3×10 ml). The resultant residue was purified by silica gel chromatography (0-10% methanol in dichloromethane) to afford the title compound as colorless glass (116 mg, 48%). LCMS (m/z) for C₄₈H₇₂N₄O₁₅⁺ (M+H)⁺ 921.8; retention time=0.91 min (UPLC 1.3 min run).

### Step 2: Example 36

10% Pd/C (25 mg, wet) was added under nitrogen to a solution of benzyl5,11,18-trioxo-1-{[3,4,6-tri-O-acetyl-2-(acetylamino)-2-deoxy-β-D-galactopyranosyl]oxy}-14-oxa-6,10,17-triazanonacosan-29-oate (196 mg, 0.213 mmol) in methanol (5 mL). The reaction was placed in the HelCat under 50 psi hydrogen and stirred at ambient temperature for 16 h. The head space was purged with nitrogen (×3) and the solution was filtered through a 0.2 um nylon syringe filter. The filter was washed with methanol and the combined filtrates were concentrated to afford the title compound as a colorless glass which was then dissolved in a mixture of acetonitrile and water (1:1, 20 mL) and freeze dried to afford a white solid (165 mg, 93%). ¹H NMR (400 MHz, CD₃OD) δ ppm 8.01-7.91 (m, 1H), 5.33 (d, 1H), 5.06 (m, 1H), 4.55 (d, 1H), 4.19-3.98 (m, 4H), 3.91-3.84 (m, 1H), 3.76-3.67 (m, 2H), 3.57-3.47 (m, 4H), 3.25-3.18 (m, 4H), 2.44 (t, 2H), 2.30-2.16 (m, 6H), 2.14 (s, 3H), 2.02 (s, 3H), 1.95 (s, 3H), 1.93 (s, 3H), 1.73-1.54 (m, 10H), 1.39-1.28 (m, 12H). LCMS (m/z) for C₃₉H₆₇N₄O₁₅⁺ (M+H)⁺ 831.8; retention time=1.30 min (UPLC 3.0 min run.

### Example 37. Synthesis of Oligonucleotides

Synthesis of various oligonucleotides is described herein. The two digits following the decimal after the WV oligonucleotide designation indicate a batch number. For example, WV-7107.03 indicates batch 03 of WV-7107.

### Example 37A. Synthesis of WV-7107 and WV-6558

WV-6558 which has the sequence 5′-Mod001L001Aeo*SGeom5CeoTeoTeo*RC*ST*ST*SG*RT*SC*SC*RA*SG*SC*RTeoTeoTeoAeo*S Teo-3′ is a GalNAc conjugate of WV-7107 which has the sequence 5′-L001Aeo*SGeom5CeoTeoTeo*RC*ST*ST*SG*RT*SC*SC*RA*SG*SC*RTeoTeoTeoAeo*STeo-3′. The GalNAc conjugation step is performed on WV-7107 to make WV-6558.

### Solid Phase Synthesis of WV-7107:

Synthesis of WV-7107 was performed on an ÄKTA OP100 synthesizer (GE healthcare) using a 6.0 cm diameter stainless steel column reactor on a 3300 μmol scale using CPG support (Loading 72 umol/g). The process consists of five steps; detritylation, coupling, capping 1, oxidation/thiolation and capping 2. Detritylation was performed using 3% DCA in toluene with a UV watch command set at 436 nm. Following detritylation, at least 4 column volumes (CV) of ACN was used to wash off the detritylation reagent.

All phosphoramidite and activator solutions (CMIMT and ETT) were prepared and dried over 3 Å molecular sieves for at least 4 hours prior to synthesis.

Stereo-defined amidite coupling was performed using 0.2 M amidite solutions and 0.6 M CMIMT. All amidites were dissolved in ACN except dC-L and dC-D amidites which were dissolved in isobutyronitrile (IBN). Stereo-defined MOE amidites were dissolved in 20% IBN/ACN v/v. CMIMT was dissolved in ACN. Using 4 equivalents, coupling was performed by mixing 40% (by volume) of the respective amidite solution with 67% of the CMIMT activator in-line prior to addition to the column. The coupling mixture was then recirculated for a minimum of 10 minutes to maximize the coupling efficiency.

Standard stereorandom amidite coupling was performed using 0.2 M amidite solutions and 0.6 M ETT in ACN. MOE-T amidite was dissolved 20% IBN/ACN v/v. Using 4 equivalents, coupling was performed by mixing 40% (by volume) of the respective amidite solution with 60% of the ETT activator in-line prior to addition to the column. The coupling mixture was then recirculated for a minimum of 6 minutes to maximize the coupling efficiency.

After coupling in both instances, the column was washed with 2CV of ACN.

For stereo-defined couplings, the column was then treated with Capping 1 solution (Acetic Anhydride, Lutidine, ACN) mixture for 1 CV to in 4 minutes acetylate the Chiral axillary amine. Following this step the column was washed with ACN for at least 2 CV. Thiolation was then performed with 0.2 M Xanthane Hydride in pyridine with a contact time of 6 min for 2 CV. After a 2 CV thiolation wash step using ACN, capping 2 was performed using 0.5 CV of Capping A and Capping B reagents mixed inline (1:1) followed by a 2 CV ACN wash.

For stereorandom coupling cycles, there is no Capping 1 step. Oxidation was performed using 50 mM Iodine in/Pyridine/H2O (9:1) for 2.5 min and 3.5 equivalents. After a 2CV ACN wash, capping 2 was performed using 0.5 CV of Capping A and Capping B reagents mixed inline (1:1) followed by a 2 CV ACN wash.

### Cleavage and Deprotection of WV-7107:

67% (or 2200 μmol) of the material synthesized above was used in this step. The DPSE protecting groups on WV-7107 were removed by treating the oligo bound solid support with a 1M solution of TEA.HF made by mixing DMSO, Water, TEA and TEA.3HF in a v/v ratio of 39:8:1:2.5, to make a 100 mL solution per mmol of oligo. The mixture was then shaken at 25° C. for 6 hours in an incubator shaker. The mixture was cooled (ice bath) then 200 mL of aqueous ammonia per mmol of oligo added. The mixture was then shaken at 45° C. for 16 hours. The mixture was then filtered (0.2-1.2 μm filters) and the cake rinsed with water. The filtrate liquor was obtained and analyzed by UPLC and a purity of 30.8% FLP obtained. Quantitation was done using a Nano Drop one spectrophotometer (Thermo Scientific) and a yield of about 101,200 OD/mmol obtained.

### Purification and Desalting of WV-7107:

The crude WV-7107 loaded on to an Agilent Load & Lock column (5 cm×32 cm) packed with Source 15Q (GE healthcare). Purification was performed on an ÄKTA 150 Pure (GE Healthcare) using 20 mM NaOH and 2.5 M NaCl as eluents. Fractions were analyzed and pooled to obtain material with a purity ≥70%. The purified material was then desalted on 2K re-generated cellulose membranes followed by lyophilization to obtain WV-7107 as a white powder. This material was then used for conjugation experiments.

### Example 37B. Synthesis of WV-6558

### Protocol for GalNAc Conjugation

Precursor material: WV-7107.03

Final Conjugated product: WV-6558.03

Reagents for Conjugation

| | | Equivalent | | | |
|---|---|---|---|---|---|
| Oligonucleotide/ | | to Oligo- | | | |
| Reagents | MW | nucleotide | mg | μL | μmole |
| WV-7107.03 | 7191.7 | 1 | 400 | — | 55.62 |
| Tri-antennary GalNAc | 2005 | 1.6 | 178.4 | — | 88.99 |
| Lot: GL-N12-26 | | | | | |
| HATU | 382 | 1.4 | 29.75 | — | 77.87 |
| P/N Sigma 445460 | | | | | |
| Lot: MKBV8272V | | | | | |
| DIEA | 129 | 10.0 | — | 98.83 | 556.2 |
| P/N Sigma 387649 | | | | | |
| Lot: SHBG2052V | | | | | |
| Acetonitrile | — | — | — | 4000 | — |

TABLE 2
Aqueous Oligonucleotide Solution

| Oligonucleotide/ | Conc | Total volume | |
|---|---|---|---|
| Solvent | (mg/mL) | (mL) | Total mg |
| WV-7107.01 in water | 50 | 8 | 400 |

Weighed 1-(((2R,3R,4R,5R,6R)-3-acetamido-4,5-diacetoxy-6-(acetoxymethyl)tetrahydro-2H-pyran-2-yl)oxy)-16,16-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic acid (1.6 eq), and HATU (1.4 eq.) and transferred to a 50 ml plastic tube. Dissolved the material in anhydrous acetonitrile then add DIEA (d=0.742) (10 eq) into the tube. The clear mixture was stirred for 20 min at 37° C. Reconstituted the lyophilized WV-7107 sample with 8 mL water to a concentration at 50 mg/mL. Then the GalNac mixture was added to sample WV-7107 and stirred for 60 min at 37° C. The progress of the reaction was monitored by UPLC. The reaction is complete after 1h of incubation. The solution was concentrated under vacuum (by speed vac) to remove acetonitrile and the resultant GalNAc-conjugated oligo was treated with concentrated Ammonium hydroxide (5 mL) for deprotection by incubating for 1 h at 37° C. The formation of the final product WV-6558 was confirmed by UPLC and Mass Spectrometry. The conjugated samples were purified by anion exchange chromatography. Observed Mass: 8802.4 (Deconvoluted), Target Mass: 8801.6.

### Example 37C. Synthesis of WV-9542

### Protocol for PFE Conjugation

Precursor material: WV-7107.02

Final Conjugated product: WV-9542.01

### Reagents for Conjugation

| | | Equivalent | | | |
|---|---|---|---|---|---|
| Oligonucleotide/ | | to Oligo- | | | |
| Reagents | MW | nucleotide | mg | μL | μmole |
| WV-7107.02 | 7191.7 | 1 | 1700 | — | 236.38 |
| Tri-antennary PFE | 2065.8 | 1.6 | 781.3 | — | 378.21 |
| ASGPR ligand | | | | | |
| Lot: GL-N12-58 | | | | | |
| HATU | 382 | 1.2 | 108.36 | — | 283.66 |
| P/N Sigma 445460 | | | | | |
| Lot: MKBV8272V | | | | | |
| DIEA | 129 | 10.0 | 304.93 | 420.02 | 2363.84 |
| P/N Sigma 387649 | | | | | |
| Lot: SHBG2052V | | | | | |
| DMF | — | — | — | 13000 | — |

TABLE 2
Aqueous Oligonucleotide Solution

| Oligonucleotide/ | Conc | Total volume | |
|---|---|---|---|
| Solvent | (mg/mL) | (mL) | Total mg |
| WV-7107.01 in water | 60 | 13 | 1800 |

Weighed Tri-antennary PFE ASGPR ligand (18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazahentriacontan-31-oic acid) (1.6 eq), and HATU (1.2 eq.) and transferred to a 50 mL tube. Dissolved the material in anhydrous Dimethylformamide then add DIEA (d=0.742) (10 eq) into the tube. The solution was sonicated till it became clear, and it was stirred for 20 min at 37° C. Reconstituted WV-7107 sample with 13 mL water. Then the Tri-antennary PFE ligand mixture was added to sample WV-7107 and stirred for 1 hr at 37° C. The progress of the reaction was monitored by UPLC. The reaction was incomplete after 1 hr incubation. Second addition of Tri-antennary PFE ligand (1.2 eq) and HATU (1 eq) were weighed out and dissolved in 5 mL DMF with DIEA (15 eq). Incubated the ligand for 20 min at 37° C. for activation. Then added the activated ligand to the reaction mixture and incubated for 1 hr at 37° C. The reaction completed and the formation of the final product WV-9542 was confirmed by UPLC and Mass Spectrometry. The conjugated samples were purified by anion exchange chromatography. Observed Mass: 8837.6 (Deconvoluted), Target Mass: 8837.6.

### Example 37D. Synthesis of WV-9543

### Protocol for PFE Conjugation

Precursor material: WV-7107.02

Final Conjugated product: WV-9543.01

### Reagents for Conjugation

| | | Equivalent | | | |
|---|---|---|---|---|---|
| Oligonucleotide/ | | to Oligo- | | | |
| Reagents | MW | nucleotide | mg | μL | μmole |
| WV-7107.02 | 7191.7 | 1 | 90 | — | 12.51 |
| Bis-antennary GalNAc | 1418.59 | 2 | 35.5 | — | 25.03 |
| Lot: PF-07075575 | | | | | |
| HATU | 382 | 1.8 | 8.6 | — | 22.53 |
| P/N Sigma 445460 | | | | | |
| Lot: MKBV8272V | | | | | |
| DIEA | 129 | 10.0 | 16.14 | 22.24 | 125.14 |
| P/N Sigma 387649 | | | | | |
| Lot: SHBG2052V | | | | | |
| Dimethylformamide | — | — | — | 1500 | — |

TABLE 2
Aqueous Oligonucleotide Solution

| Oligonucleotide/ | Conc | Total volume | |
|---|---|---|---|
| Solvent | (mg/mL) | (mL) | Total mg |
| WV-7107.01 in water | 60 | 1.5 | 90 |

Weighed the Bis-antennary GalNAc (1-(((2R,3R,4R,5R,6R)-3-acetamido-4,5-diacetoxy-6-(acetoxymethyl)tetrahydro-2H-pyran-2-yl)oxy)-16-((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic acid) (2.0 eq), and HATU (1.8 eq) and transferred to a 50 mL tube. Dissolved the material in anhydrous Dimethylformamide (1.5 mL) then add DIEA (d=0.742) (10 eq) into the tube. The solution was sonicated till it became clear, and it was stirred for 20 min at 37° C. Reconstituted WV-7107 sample with 1.5 mL water. Then the Bis-antennary GalNAc mixture was added to sample WV-7107 and stirred for 1 hr at 37° C. The progress of the reaction was monitored by UPLC. The reaction was completed after 1 hr incubation. The mixture was treated with concentrated Ammonium hydroxide (2 mL) for deprotection by incubating for 1 h at 37° C. Formation of the final product WV-9543 was confirmed by UPLC and Mass Spectrometry. The conjugated samples were purified by anion exchange chromatography. Observed Mass: 8342.6 (Deconvoluted), Target Mass: 8340.1.

### Example 37E. Synthesis of WV-9544

### Protocol for PFE Conjugation

Precursor material: WV-7107.02

Final Conjugated product: WV-9544.01

### Reagents for Conjugation

| | | Equivalent | | | |
|---|---|---|---|---|---|
| Oligonucleotide/ | | to Oligo- | | | |
| Reagents | MW | nucleotide | mg | μL | μmole |
| WV-7107.02 | 7191.7 | 1 | 90 | — | 12.51 |
| Bis-antennary PFE | 1190.39 | 2 | 29.8 | — | 25.03 |
| ASGPR ligand | | | | | |
| Lot: PF-07075667 | | | | | |
| HATU | 382 | 1.8 | 8.6 | — | 22.53 |
| P/N Sigma 445460 | | | | | |
| Lot: MKBV8272V | | | | | |
| DIEA | 129 | 10.0 | 16.14 | 22.24 | 125.14 |
| P/N Sigma 387649 | | | | | |
| Lot: SHBG2052V | | | | | |
| Dimethylformamide | — | — | — | 1500 | — |

TABLE 2
Aqueous Oligonucleotide Solution

| Oligonucleotide/ | Conc | Total volume | |
|---|---|---|---|
| Solvent | (mg/mL) | (mL) | Total mg |
| WV-7107.01 in water | 60 | 1.5 | 90 |

Weighed the Bis-antennary PFE ASGPR ligand (18-(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazahentriacontan-31-oic acid) (2.0 eq), and HATU (1.8 eq) and transferred to a 50 mL tube. Dissolved the material in anhydrous Dimethylformamide (1.5 mL) then add DIEA (d=0.742) (10 eq) into the tube. The solution was sonicated till it became clear, and it was stirred for 20 min at 37° C. Reconstituted WV-7107 sample with 1.5 mL water. Then the Bis-antennary PFE ASGPR ligand mixture was added to sample WV-7107 and stirred for 1 hr at 37° C. The progress of the reaction was monitored by UPLC. The reaction was completed after 1 hr incubation. Formation of the final product WV-9544 was confirmed by UPLC and Mass Spectrometry. The conjugated samples were purified by anion exchange chromatography. Observed Mass: 8367.2 (Deconvoluted), Target Mass: 8364.1.

### Example 37F. Synthesis of WV-9545

### Protocol for PFE Conjugation

Precursor material: WV-7107.02

Final Conjugated product: WV-9545.01

### Reagents for Conjugation

| | | Equivalent | | | |
|---|---|---|---|---|---|
| Oligonucleotide/ | | to Oligo- | | | |
| Reagents | MW | nucleotide | mg | μL | μmole |
| WV-7107.02 | 7191.7 | 1 | 90 | — | 12.51 |
| Mono GalNAc | 830.97 | 2 | 20.8 | — | 25.03 |
| Lot: PF-07075574 | | | | | |
| HATU | 382 | 1.8 | 8.6 | — | 22.53 |
| P/N Sigma 445460 | | | | | |
| Lot: MKBV8272V | | | | | |
| DIEA | 129 | 10.0 | 16.14 | 22.24 | 125.14 |
| P/N Sigma 387649 | | | | | |
| Lot: SHBG2052V | | | | | |
| Dimethylformamide | — | — | — | 1500 | — |

TABLE 2
Aqueous Oligonucleotide Solution

| Oligonucleotide/ | Conc | Total volume | |
|---|---|---|---|
| Solvent | (mg/mL) | (mL) | Total mg |
| WV-7107.01 in water | 60 | 1.5 | 90 |

Weighed the Mono GalNAc (1-(((2R,3R,4R,5R,6R)-3-acetamido-4,5-diacetoxy-6-(acetoxymethyl)tetrahydro-2H-pyran-2-yl)oxy)-5,11,18-trioxo-14-oxa-6,10,17-triazanonacosan-29-oic acid) (2.0 eq), and HATU (1.8 eq) and transferred to a 50 mL tube. Dissolved the material in anhydrous Dimethylformamide (1.5 mL) then add DIEA (d=0.742) (10 eq) into the tube. The solution was sonicated till it became clear, and it was stirred for 20 min at 37° C. Reconstituted WV-7107 sample with 1.5 mL water. Then the Mono GalNAc ligand mixture was added to sample WV-7107 and stirred for 1 hr at 37° C. The progress of the reaction was monitored by UPLC. The reaction was completed after 1 hr incubation. The mixture was treated with concentrated Ammonium hydroxide (2 mL) for deprotection by incubating for 1 h at 37° C. Formation of the final product WV-9545 was confirmed by UPLC and Mass Spectrometry. The conjugated samples were purified by anion exchange chromatography. Observed Mass: 7881.3 (Deconvoluted), Target Mass: 7878.6.

### Example 37G. Synthesis of WV-9546

### Protocol for PFE Conjugation

Precursor material: WV-7107.02

Final Conjugated product: WV-9546.01

### Reagents for Conjugation

| | | Equivalent | | | |
|---|---|---|---|---|---|
| Oligonucleotide/ | | to Oligo- | | | |
| Reagents | MW | nucleotide | mg | μL | μmole |
| WV-7107.02 | 7191.7 | 1 | 90 | — | 12.51 |
| Mono PFE ASGPR | 716.87 | 2 | 17.9 | — | 25.03 |
| ligand | | | | | |
| Lot: PF-07075666 | | | | | |
| HATU | 382 | 1.8 | 8.6 | — | 22.53 |
| P/N Sigma 445460 | | | | | |
| Lot: MKBV8272V | | | | | |
| DIEA | 129 | 10.0 | 16.14 | 22.24 | 125.14 |
| P/N Sigma 387649 | | | | | |
| Lot: SHBG2052V | | | | | |
| Dimethylformamide | — | — | — | 1500 | — |

TABLE 2
Aqueous Oligonucleotide Solution

| Oligonucleotide/ | Conc | Total volume | |
|---|---|---|---|
| Solvent | (mg/mL) | (mL) | Total mg |
| WV-7107.01 in water | 60 | 1.5 | 90 |

Weighed the Mono PFE ASGPR ligand (1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazahentriacontan-31-oic acid) (2.0 eq), and HATU (1.8 eq) and transferred to a 50 mL tube. Dissolved the material in anhydrous Dimethylformamide (1.5 mL) then add DIEA (d=0.742) (10 eq) into the tube. The solution was sonicated till it became clear, and it was stirred for 20 min at 37° C. Reconstituted WV-7107 sample with 1.5 mL water. Then the Mono GalNAc ligand mixture was added to sample WV-7107 and stirred for 1 hr at 37° C. The progress of the reaction was monitored by UPLC. The reaction was completed after 1 hr incubation. Formation of the final product WV-9546 was confirmed by UPLC and Mass Spectrometry. The conjugated samples were purified by anion exchange chromatography. Observed Mass: 7893.1 (Deconvoluted), Target Mass: 7890.6.

### Example 37H. IEX Purification Condition

### For Sample WV-9542

| Buffer A | 20 mM Sodium Hydroxide | |
|---|---|---|
| Buffer B | 2.5N sodium chloride in 20 mM Sodium hydroxide | |
| Column | 2.5 cm × 33 cm Source 15Q | |
| Gradient | % B | Column Vol (160 mL) |
| | 0 | 2 |
| | 0-15 | 2 |
| | 15 | 1 |
| | 15-90 | 15 |
| | 100 | 1 |

### For Sample WV-6558, WV-9542-WV-9546

| Buffer A | 20 mM Sodium Hydroxide | |
|---|---|---|
| Buffer B | 2.5N sodium chloride in 20 mM Sodium hydroxide | |
| Column | 2.0 cm × 10 cm Source 15Q | |
| Gradient | % B | Column Vol (160 mL) |
| | 0 | 2 |
| | 0-20 | 5 |
| | 20 | 1 |
| | 20-90 | 15 |
| | 100 | 1 |

### Example 38. Synthesis of Ligand

### Synthesis of 1-(((2R,3R,4R,5R,6R)-3-acetamido-4,5-diacetoxy-6-(acetoxymethyl)tetrahydro-2H-pyran-2-yl)oxy)-16,16-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic acid

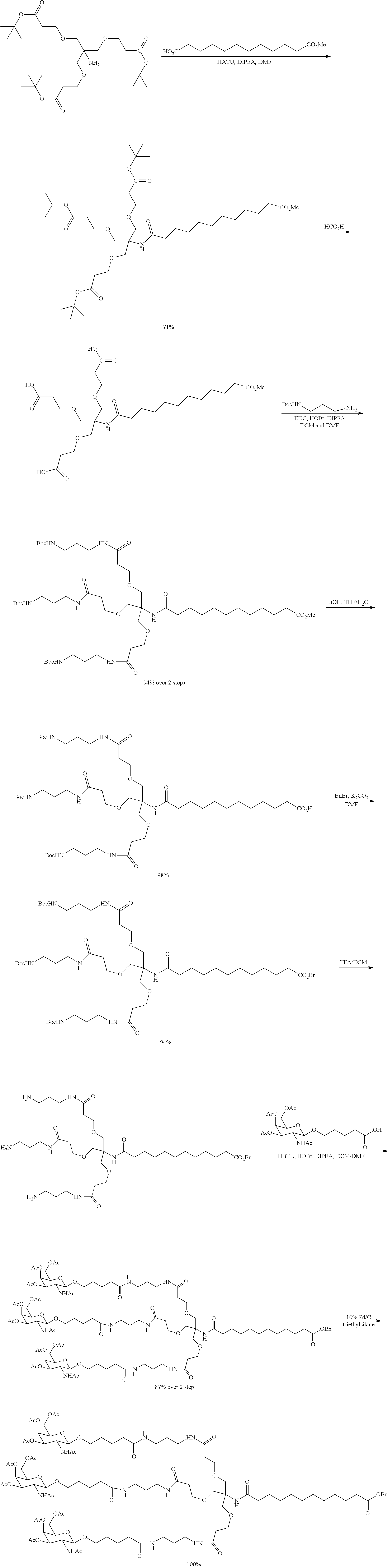

### Step 1

To a solution of di-tert-butyl 3,3′-((2-amino-2-((3-(tert-butoxy)-3-oxopropoxy)methyl)propane-1,3-diyl)bis(oxy))dipropanoate (5.0 g, 9.89 mmol) and 12-methoxy-12-oxododecanoic acid (2.416 g, 9.89 mmol) in DMF (45 mL) was added HATU (3.76 g, 9.89 mmol) and DIPEA (2.58 ml, 14.83 mmol). The reaction mixture was stirred at room temperature for 5 hrs. Solvent was concentrated under reduced pressure, and diluted with brine, extracted with EtOAc, dried over anhydrous sodium sulfate, and concentrated to give a residue, which was purified by ISCO (120 g gold silica gel cartridge) eluting with 10% EtOAc in hexane to 40% EtOAc in hexane to give di-tert-butyl 3,3′-((2-((3-(tert-butoxy)-3-oxopropoxy)methyl)-2-(12-methoxy-12-oxododecanamido)propane-1,3-diyl)bis(oxy))dipropanoate (5.13 g, 7.01 mmol, 70.9% yield) as a colorless oil. ¹H NMR (400 MHz, Chloroform-d) δ 6.03 (s, 1H), 3.74-3.61 (m, 15H), 2.45 (t, J=6.3 Hz, 6H), 2.31 (td, J=7.5, 3.9 Hz, 2H), 2.19-2.10 (m, 2H), 1.64-1.59 (m, 4H), 1.46 (s, 27H), 1.32-1.24 (m, 12H); MS (ESI), 732.6 (M+H)⁺.

### Step 2

A solution of di-tert-butyl 3,3′-((2-((3-(tert-butoxy)-3-oxopropoxy)methyl)-2-(12-methoxy-12-oxododecanamido)propane-1,3-diyl)bis(oxy))dipropanoate (5.0 g, 6.83 mmol) in formic acid (50 mL) was stirred at room temperature for 48 hrs. Solvent was evaporated under reduced pressure, co-evaporated with toluene (3×) to give a white solid, which was dried under high vacuum for 2 days. LC-MS and H NMR showed the reaction is not complete. The crude product was redissolved in formic acid (50 mL). The reaction mixture was stirred at room temperature for 24 hrs. LC-MS showed the reaction was complete. Solvent was evaporated under reduced pressure, co-evaporated with toluene (3×), dried over high vacuum to give 3,3′-((2-((2-carboxyethoxy)methyl)-2-(12-methoxy-12-oxododecanamido)propane-1,3-diyl)bis(oxy))dipropanoic acid (4.00 g) as a white solid. MS (ESI): 562.4 (M−H)⁻.

### Step 3

A solution of 3,3′-((2-((2-carboxyethoxy)methyl)-2-(12-methoxy-12-oxododecanamido)propane-1,3-diyl)bis(oxy))dipropanoic acid (3.85 g, 6.83 mmol) and HOBt (3.88 g, 28.7 mmol) in DCM (60 mL) and DMF (15 mL) at 0° C. was added tert-butyl (3-aminopropyl)carbamate (4.76 g, 27.3 mmol), EDAC HCl salt (5.24 g, 27.3 mmol) and DIPEA (8.33 ml, 47.8 mmol). The reaction mixture was stirred at 0° C. for 15 minutes and at room temperature for 20 hrs. LC-MS showed the reaction was not complete. t-Butyl (3-mainopropyl) carbamate (1.59 g, 9.12 mmol) and EDC HCl salt (1.75 g, 9.13 mol) was added into the reaction mixture. The reaction mixture was continually stirred at room temperature for 4 hrs. Solvent was evaporated to give a residue, which was dissolved in EtOAc (300 mL), washed with water (1×), saturated sodium bicarbonate (2×), 10% citric acid (2×) and water, dried over sodium sulfate, and concentrated to give a residue which was purified by ISCO (80 g gold cartridge) eluting with DCM to 30% MeOH in DCM to give methyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oate (6.61 g, 6.40 mmol, 94% yield over 2 steps) as a white solid. MS (ESI): 1033.5 (M+H)+.

### Step 4

To a solution of methyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oate (6.56 g, 6.35 mmol) in THF (75 mL) was added aq. LiOH (0.457 g, 19.06 mmol) in water (25 mL). The mixture was stirred at room temperature for overnight. LC-MS showed the reaction was completed. Solvent was evaporated, acidified using 1 N HCl (45 mL), extracted with DCM (3×), dried over anhydrous sodium sulfate, concentrated to give 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oic acid (6.31 g, 6.20 mmol, 98% yield) as a white solid. MS (ESI): 1019.6 (M+H)⁺.

### Step 5

To a solution of 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oic acid (6.31 g, 6.20 mmol) and (bromomethyl)benzene (1.272 g, 7.44 mmol) in DMF (40 mL) was added K₂CO₃ (2.57 g, 18.59 mmol). The mixture was stirred at 40° C. for 4 hrs and at room temperature for overnight. Solvent was evaporated under reduced pressure. The reaction mixture was diluted with EtOAc, washed with water, dried over anhydrous sodium sulfate, concentrated under reduced pressure to give a residue, which was purified by ISCO (80 g cartridge) eluting with DCM to 30% MeOH in DCM to give benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oate (6.41 g, 5.78 mmol, 93% yield) as a colorless oil. ¹H NMR (400 MHz, DMSO-d₆) δ 7.80 (t, J=5.7 Hz, 3H), 7.39-7.30 (m, 5H), 6.95 (s, 1H), 6.74 (t, J=5.8 Hz, 3H), 5.07 (s, 2H), 3.53 (J, J=7.3 Hz, 6H), 3.51 (s, 6H), 3.02 (q, J=6.7 Hz, 6H), 2.94-2.85 (m, 6H), 2.29 (dt, J=26.1, 6.9 Hz, 8H), 2.02 (q, J=9.7, 8.6 Hz, 2H), 1.56-1.39 (m, 10H), 1.35 (s, 27H), 1.20 (brs, 14H); MS (ESI): 1019.6 (M+H)+.

### Step 6

To a solution of benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazaoctacosan-28-oate (2.42 g, 2.183 mmol) in DCM (40 mL) was added 2,2,2-trifluoroacetic acid (8 ml, 105 mmol). The reaction mixture was stirred at room temperature for overnight. Solvent was evaporated under reduced pressure, co-evaporated with toluene (2×), triturated with ether, dried under high vacuum for overnight. Directly use TFA salt for next step.

### Step 7

To a solution of 5-(((2R,3R,4R,5R,6R)-3-acetamido-4,5-diacetoxy-6-(acetoxymethyl)tetrahydro-2H-pyran-2-yl)oxy)pentanoic acid (3.91 g, 8.73 mmol), HBTU (3.48 g, 9.17 mmol) and HOBT (1.239 g, 9.17 mmol) in DCM (25 mL) was added DIPEA (6.08 ml, 34.9 mmol) followed by benzyl 12-((1,19-diamino-10-((3-((3-aminopropyl)amino)-3-oxopropoxy)methyl)-5,15-dioxo-8,12-dioxa-4,16-diazanonadecan-10-yl)amino)-12-oxododecanoate (1.764 g, 2.183 mmol) in DMF (4.0 mL). The mixture was stirred at room temperature for 5 hrs. Solvent was evaporated under reduced pressure to give a residue, which was purified by ISCO (40 g gold column) eluting with 5% MeOH in DCM for 5 column value to remove HOBt followed by 5% to 30% MeOH in DCM to give 1-(((2R,3R,4R,5R,6R)-3-acetamido-4,5-diacetoxy-6-(acetoxymethyl)tetrahydro-2H-pyran-2-yl)oxy)-16,16-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic benzyl ester (3.98 g, 87% yield) as a white solid. ¹H NMR (400 MHz, DMSO-d₆) δ 7.82-7.74 (m, 6H), 7.69 (t, J=5.6 Hz, 3H), 7.33-7.27 (m, 5H), 6.94 (s, 1H), 5.16 (d, J=3.4 Hz, 3H), 5.03 (s, 2H), 4.92 (dd, J=11.2, 3.4 Hz, 3H), 4.43 (d, J=8.4 Hz, 3H), 4.02-3.95 (m, 9H), 3.82 (dt, J=11.2, 8.8 Hz, 3H), 3.65 (dt, J=10.5, 5.6 Hz, 3H), 3.51-3.44 (m, 12H), 3.36 (dt, J=9.6, 6.0 Hz, 3H), 3.01-2.95 (m, 12H), 2.29 (t, J=7.4 Hz, 2H), 2.23 (t, J=6.3 Hz, 6H), 2.05 (s, 9H), 1.99 (t, J=7.0 Hz, 8H), 1.94 (s, 9H), 1.84 (s, 9H), 1.72 (s, 9H), 1.50-1.14 (m, 34H); MS (ESI): 1049.0 (M/2+H)+.

### Step 8

To a round bottom flask flushed with Ar was added 10% Pd/C (165 mg, 0.835 mmol) and EtOAc (15 mL). A solution of Benzyl protected tris-GalNAc (1.75 g, 0.835 mmol) in methanol (15 mL) was added followed by triethylsilane (2.67 ml, 16.70 mmol) dropwise. The mixture was stirred at room temperature for 3 hrs. LC-MS showed the reaction was complete, diluted with EtOAc, and filtered through celite, washed with 20% MeOH in EtOAc, concentrated under reduced pressure to give 1-(((2R,3R,4R,5R,6R)-3-acetamido-4,5-diacetoxy-6-(acetoxymethyl)tetrahydro-2H-pyran-2-yl)oxy)-16,16-bis((3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)methyl)-5,11,18-trioxo-14-oxa-6,10,17-triazanonacosan-29-oic acid (1.67 g, 0.832 mmol, 100% yield) as a white solid. ¹H NMR (400 MHz, DMSO-d₆) δ 11.95 (s, 1H), 7.83-7.74 (m, 6H), 7.69 (t, J=5.7 Hz, 3H), 6.93 (s, 1H), 5.16 (d, J=3.4 Hz, 3H), 4.92 (dd, J=11.2, 3.4 Hz, 3H), 4.43 (d, J=8.4 Hz, 3H), 4.01-3.94 (m, 9H), 3.82 (dt, J=11.3, 8.8 Hz, 3H), 3.66 (dt, J=10.7, 5.6 Hz, 3H), 3.54-3.43 (m, 12H), 3.41-3.33 (m, 3H), 3.03-2.94 (m, 12H), 2.24 (t, J=7.4 Hz, 10H), 2.14 (t, J=7.4 Hz, 2H), 2.06 (s, 9H), 2.00 (t, J=7.2 Hz, 8H), 1.95 (s, 9H), 1.84 (s, 9H), 1.73 (s, 9H), 1.51-1.14 (m, 34H). MS (ESI): 1003.8 (M/2+H)+.

### Example 39. Synthesis of Ligand

### Synthesis of 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazatetracosan-24-oic acid

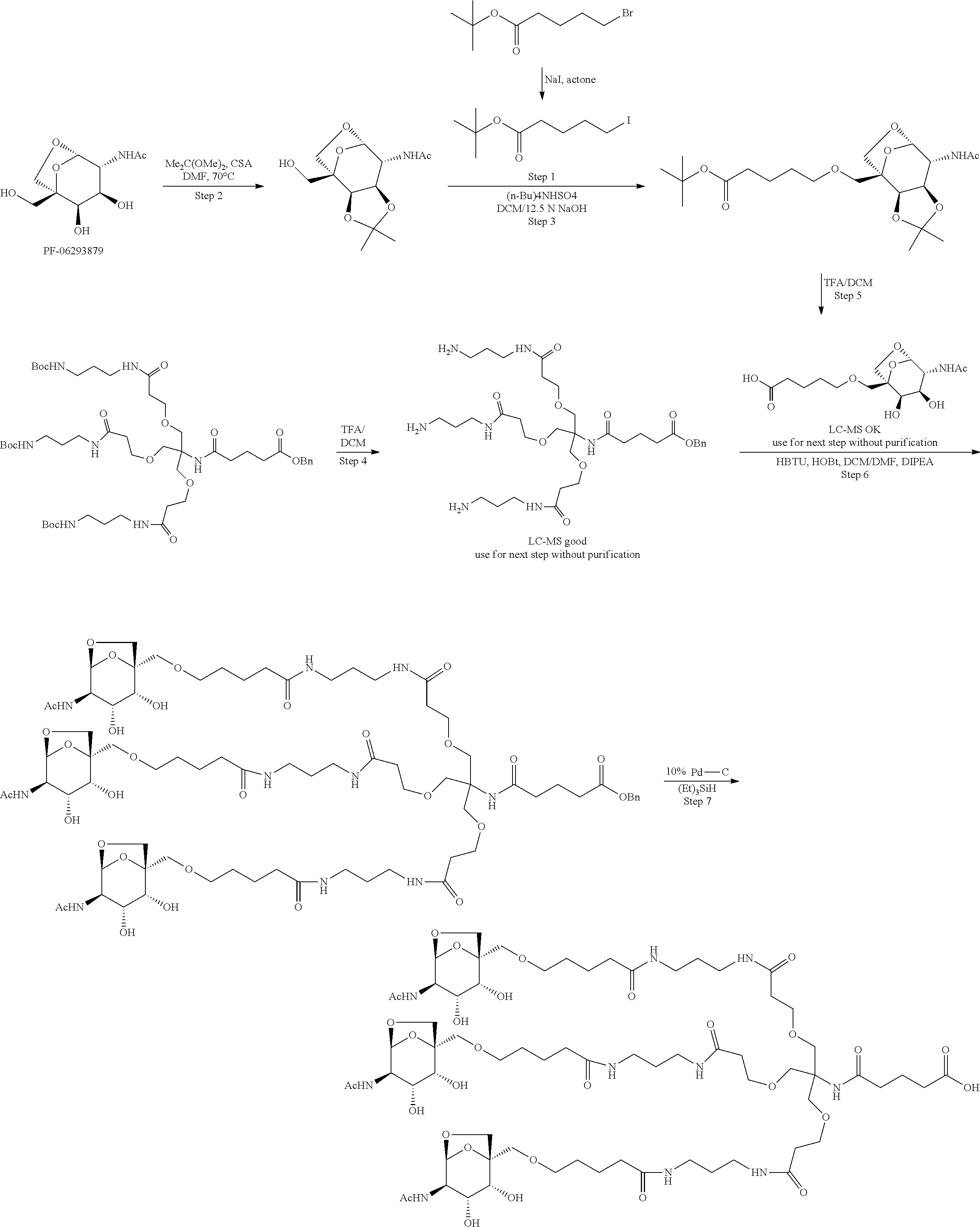

### Step 1

To a solution of tert-butyl 5-bromopentanoate (4.0 g, 16.87 mmol) in acetone (80 mL) was added NaI (7.59 g, 50.6 mmol). The reaction mixture was stirred at 57° C. for 2 hrs, filtered, and washed with EtOAc. Solvent was evaporated under reduced pressure to give a residue, which was dissolved in EtOAc, washed with water, brine, dried over Na₂SO₄, concentrated to give a residue, which was purified by ISCO (40 g column) eluting with 20% EtOAc in hexane to 50% EtOAc in hexane to give tert-butyl 5-iodopentanoate 6 (4.54 g, 15.98 mmol, 95% yield) as a yellow oil. ¹H NMR (500 MHz, Chloroform-d) δ 3.19 (t, J=6.9 Hz, 2H), 2.24 (t, J=7.3 Hz, 2H), 1.86 (p, J=7.1 Hz, 2H), 1.70 (p, J=7.4 Hz, 2H), 1.45 (s, 9H).

### Step 2

To a solution of N-((1S,2R,3R,4R,5S)-2,3-dihydroxy-1-(hydroxymethyl)-6,8-dioxabicyclo[3.2.1]octan-4-yl)acetamide (600 mg, 2.57 mmol) in DMF (15 mL) was added 2,2-dimethoxypropane (2087 μl, 17.03 mmol) followed by (+/−)-camphor-10-sulphonic acid (264 mg, 1.135 mmol). The reaction mixture was stirred at 70° C. for 24 hrs. The reaction mixture was cooled down to room temperature, and then methanol (2.5 mL) was added. The reaction mixture was stirred at room temperature for 30 minutes and neutralized with TEA (0.10 mL). The solvent was evaporated and the residue was coevaporated with toluene. The residue was purified by ISCO (24 g gold) eluting with EtOAc to 10% MeOH in EtOAc to give N-((3aR,4S,7S,8R,8aR)-4-(hydroxymethyl)-2,2-dimethylhexahydro-4,7-epoxy[1,3]dioxolo[4,5-d]oxepin-8-yl)acetamide 7 (666 mg, 2.437 mmol, 95% yield). ¹H NMR (500 MHz, DMSO-d₆) δ 8.09 (d, J=8.1 Hz, 1H), 5.15-5.05 (m, 2H), 4.26 (d, J=5.8 Hz, 1H), 4.09 (dd, J=7.3, 5.8 Hz, 1H), 3.80-3.60 (m, 5H), 1.83 (s, 3H), 1.37 (s, 3H), 1.26 (s, 3H); MS, 274.3 (M+H)+.

### Step 3

To a solution of tert-butyl 5-iodopentanoate (1310 mg, 4.61 mmol) and N-((3aR,4S,7S,8R,8aR)-4-(hydroxymethyl)-2,2-dimethylhexahydro-4,7-epoxy[1,3]dioxolo[4,5-d]oxepin-8-yl)acetamide 7 (420 mg, 1.537 mmol) in DCM (10.5 mL) was added tetrabutylammonium hydrogensulfate (783 mg, 2.305 mmol) followed by 12.5 M sodium hydroxide solution (7 mL). The reaction mixture was stirred at room temperature for 24 hrs. The reaction mixture was diluted with DCM and water, extracted with DCM (2×).

The organic layer was washed with 1 N HCl solution, and dried over sodium sulfate. Solvent was concentrated under reduce pressure to give a residue. The resulting crude material was added ethyl acetate (30 mL) and sonicated for 5 minutes. The result precipitate was filtered, washed with ethyl acetate (10 mL×2). LC MS showed the filter doens't contain desired product and was tetrabutylammonium salt. The filtrate was concentrated under reduced pressure to give a residue, which was purified by ISCO (40 g silica gel gold cartridge) eluting with 50% EtOAc in hexane to EtOAc to give tert-butyl 5-(((3aR,4S,7S,8R,8aR)-8-acetamido-2,2-dimethylhexahydro-4,7-epoxy[1,3]dioxolo[4,5-d]oxepin-4-yl)methoxy)pentanoate (0.470 g, 1.094 mmol, 71.2% yield) as a yellowish oil. ¹H NMR (500 MHz, Chloroform-d) δ 5.56 (d, J=9.1 Hz, 1H), 4.21 (d, J=5.9 Hz, 1H), 4.12 (dtd, J=7.7, 3.8, 1.7 Hz, 1H), 3.99 (t, J=6.3 Hz, 1H), 3.90 (d, J=9.5 Hz, 1H), 3.77 (d, J=2.0 Hz, 2H), 3.67 (d, J=9.5 Hz, 1H), 3.52 (ddt, J=30.5, 9.2, 5.8 Hz, 2H), 2.23 (t, J=7.1 Hz, 2H), 2.03 (d, J=14.5 Hz, 3H), 1.65-1.55 (m, 7H), 1.44 (s, 9H), 1.35 (s, 3H); MS, 452.4 (M+Na)+.

### Step 4

To a solution of benzyl 15,15-bis(13,13-dimethyl-5,11-dioxo-2,12-dioxa-6,10-diazatetradecyl)-2,2-dimethyl-4,10,17-trioxo-3,13-dioxa-5,9,16-triazahenicosan-21-oate (0.168 g, 0.166 mmol) in DCM (3 mL) was added TFA (3 mL). The reaction mixture was stirred at room temperature for 3 hrs. LC-MS showed the reaction was completed. Solvent was evaporated under reduced pressure to give benzyl 5-((1,19-diamino-10-((3-((3-aminopropyl)amino)-3-oxopropoxy)methyl)-5,15-dioxo-8,12-dioxa-4,16-diazanonadecan-10-yl)amino)-5-oxopentanoate as a colorless oil. MS, 710.5 (M+H)+. Directly use for next step without purification.

### Step 5

To a solution of tert-butyl 5-(((3aR,4S,7S,8R,8aR)-8-acetamido-2,2-dimethylhexahydro-4,7-epoxy[1,3]dioxolo[4,5-d]oxepin-4-yl)methoxy)pentanoate (285 mg, 0.664 mmol) in DCM (5 mL) was added TFA (5 mL) was stirred at room temperature for 4 hrs. LC-MS showed the reaction was complete. Solvent was evaporated to give 5-(((1S,2R,3R,4R,5S)-4-acetamido-2,3-dihydroxy-6,8-dioxabicyclo[3.2.1]octan-1-yl)methoxy)pentanoic acid. MS (ESI): 334.3 (M+H)+. Directly use for next step without purification.

### Step 6

To a solution of 5-(((1S,2R,3R,4R,5S)-4-acetamido-2,3-dihydroxy-6,8-dioxabicyclo[3.2.1]octan-1-yl)methoxy)pentanoic acid (221 mg, 0.664 mmol) in DCM (10 mL) was added DIPEA (2313 μl, 13.28 mmol), HBTU (208 mg, 0.548 mmol), HOBT (67.3 mg, 0.498 mmol), a solution of benzyl 5-((1,19-diamino-10-((3-((3-aminopropyl)amino)-3-oxopropoxy)methyl)-5,15-dioxo-8,12-dioxa-4,16-diazanonadecan-10-yl)amino)-5-oxopentanoate (118 mg, 0.166 mmol) (GL08-02) in DMF (3.0 mL) and DCM (5.0 mL). The reaction mixture was stirred at room temperature for overnight. LC-MS showed the desired product. Solvent was evaporated under reduced pressure to give a residue, which was purified by ISCO (24 g gold cartridge) eluting with DCM to 80% MeOH in DCM to give benzyl 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazatetracosan-24-oate (272 mg, 0.164 mmol, 99% yield) (product @ tube 30 to 42 (40% MeOH in DCM to 60% MeOH in DCM)

¹H NMR (500 MHz, DMSO-d₆) δ 7.89 (d, J=7.8 Hz, 3H), 7.81 (t, J=5.7 Hz, 3H), 7.75 (s, 3H), 7.34 (q, J=7.5, 6.9 Hz, 5H), 7.05 (s, 1H), 5.07 (s, 5H), 4.83 (d, J=5.3 Hz, 3H), 4.56 (d, J=7.1 Hz, 3H), 3.73 (dd, J=23.3, 9.2 Hz, 6H), 3.64 (d, J=7.0 Hz, 6H), 3.58-3.35 (m, 27H), 3.02 (p, J=6.2 Hz, 12H), 2.33 (t, J=7.6 Hz, 2H), 2.26 (t, J=6.4 Hz, 6H), 2.10 (t, J=7.6 Hz, 2H), 2.04 (t, J=7.4 Hz, 6H), 1.82 (s, 9H), 1.72 (q, J=7.6 Hz, 2H), 1.52-1.39 (m, 18H); MS (ESI), 1656.3 (M+H)⁺.

### Step 7

To a solution of benzyl 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazatetracosan-24-oate (270 mg, 0.163 mmol) in EtOAc (10 mL) was added 10% Pd—C(50 mg), and MeOH (5.0 mL), and triethylsilane (1042 μl, 6.52 mmol). The reaction mixture was stirred at room temperature for 1 hr, filtered, and concentrated to give 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazatetracosan-24-oic acid (246 mg, 0.157 mmol, 96% yield) as a white solid. ¹H NMR (500 MHz, DMSO-d₆) δ 11.99 (brs, 1H), 7.89 (d, J=7.9 Hz, 3H), 7.82 (t, J=5.4 Hz, 3H), 7.75 (t, J=5.7 Hz, 3H), 7.03 (s, 1H), 5.07 (d, J=1.6 Hz, 3H), 4.83 (brs, 3H), 4.56 (brs, 3H), 3.79-3.68 (m, 6H), 3.64 (d, J=7.2 Hz, 6H), 3.58-3.34 (m, 27H), 3.02 (p, J=6.3 Hz, 12H), 2.27 (t, J=6.4 Hz, 6H), 2.17 (t, J=7.5 Hz, 2H), 2.08 (t, J=7.5 Hz, 2H), 2.04 (t, J=7.3 Hz, 6H), 1.82 (s, 9H), 1.65 (p, J=7.5 Hz, 2H), 1.54-1.40 (m, 18H); MS(ESI), 1566.3 (M+H)+.

### Example 40. Synthesis of Ligand

### Synthesis of 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5S)-4-acetamido-2,3-dihydroxy-6,8-dioxabicyclo[3.2.1]octan-1-yl)-7,13,20-trioxo-2,16-dioxa-8,12,19-triazahentriacontan-31-oic acid

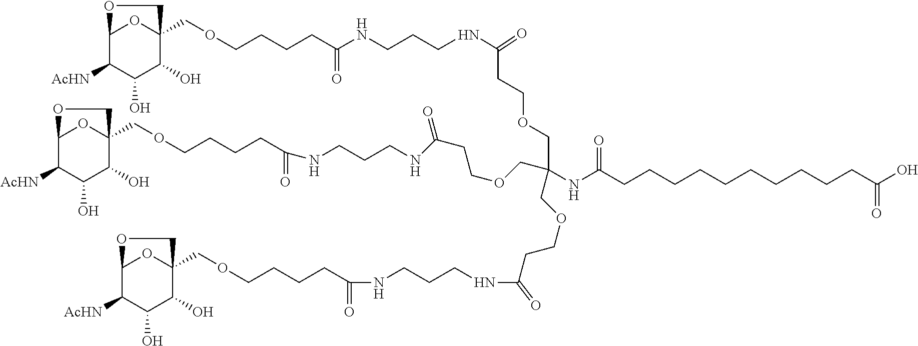 [SMILES: CC(=O)N[C@H]1[C@H]2OC[C@](COCCCCC(=O)NCCCNC(=O)CCOCC(COCCC(=O)NCCCNC(=O)CCCCOC[C@@]34CO[C@@H](O3)[C@H](NC(C)=O)[C@@H](O)[C@H]4O)(COCCC(=O)NCCCNC(=O)CCCCOC[C@@]34CO[C@@H](O3)[C@H](NC(C)=O)[C@@H](O)[C@H]4O)NC(=O)CCCCCCCCCCC(=O)O)(O2)[C@H](O)[C@@H]1O]

18,18-bis(17-((1S,2R,3R,4R,5 S)-4-acetamido-2,3-dihydroxy-6,8-dioxabicyclo[3.2.1]octan-1-yl)-5,11-dioxo-2,16-dioxa-6,10-diazaheptadecyl)-1-((1S,2R,3R,4R,5 S)-4-acetamido-2,3-dihydroxy-6,8-dioxabicyclo[3.2.1]octan-1-yl)-7,13,20-trioxo-2,16-dioxa-8,12,19-triazahentriacontan-31-oic acid was synthesized using the same procedure as 18,18-bis(17-((1S,2R,3R,4R,5S)-4-acetamido-2,3-dihydroxy-6,8-dioxabicyclo[3.2.1]octan-1-yl)-5,11-dioxo-2,16-dioxa-6,10-diazaheptadecyl)-14(1S,2R,3R,4R,5 S)-4-acetamido-2,3-dihydroxy-6,8-dioxabicyclo[3.2.1]octan-1-yl)-7,13,20-trioxo-2,16-dioxa-8,12,19-triazatetracosan-24-oic acid. ¹H NMR (400 MHz, DMSO-d₆) δ 7.90 (d, J=7.8 Hz, 3H), 7.83 (t, J=5.7 Hz, 3H), 7.76 (t, J=5.7 Hz, 3H), 6.98 (d, J=6.2 Hz, 1H), 5.09 (s, 3H), 3.81-3.69 (m, 6H), 3.69-3.62 (m, 6H), 3.62-3.40 (m, 24H), 3.04 (p, J=6.1 Hz, 9H), 2.28 (t, J=6.4 Hz, 4H), 2.18 (t, J=7.3 Hz, 2H), 2.06 (t, J=7.7 Hz, 6H), 1.84 (s, 6H), 1.48 (tq, J=14.9, 7.4 Hz, 16H), 1.23 (s, 8H). MS(ESI), 1664.0 (M+H)⁺.

### EQUIVALENTS

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives. Acts, elements, and features discussed only in connection with one embodiment are not intended to be excluded from a similar role in other embodiments. Further, for the one or more means-plus-function limitations recited, for example, in claimed inventions, if any, the means are not intended to be limited to the means disclosed herein for performing the recited function, but are intended to cover in scope any means, known now or later developed, for performing the recited function.

Use of ordinal terms such as “first”, “second”, “third”, etc., in claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, use of these terms in the specification does not by itself connote any required priority, precedence, or order. Neither does use of any such terms indicate number of elements in described (including claimed) inventions.

The foregoing written specification is sufficient to enable one skilled in the art to practice any invention described in the present disclosure. The present disclosure is not to be limited in scope by examples provided, which are intended as illustrations of one or more aspects of described inventions and other functionally equivalent embodiments are within the scope of described inventions. Various modifications of described inventions in addition to those shown and described herein will become apparent to those skilled in the art from the foregoing description and fall within the scope of described inventions. Advantages and objects of described inventions are not necessarily encompassed by each embodiment of described inventions.

The foregoing written specification is considered to be sufficient to enable one skilled in the art to practice the invention. The present disclosure is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended Embodiments. The advantages and objects of the invention are not necessarily encompassed by each embodiment of the invention.

Throughout this application, various publications are referenced. The disclosures of these publications in their entireties are hereby incorporated by reference into this application for all purposes.

1.-72. (canceled)

73. A composition comprising oligonucleotides of a particular oligonucleotide type characterized by:
a) a common base sequence;
b) a common pattern of backbone linkages;
c) a common pattern of backbone chiral centers;
wherein the oligonucleotide comprises at least one internucleotidic linkage comprising a linkage phosphorus in the Sp configuration;
wherein the composition is chirally controlled in that it is enriched, relative to a substantially racemic preparation of oligonucleotides having the same common base sequence, for oligonucleotides of the particular oligonucleotide type; and
wherein the oligonucleotide targets APOC3.

74. A composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in the composition that share the common base sequence are oligonucleotides of the plurality; and
wherein the common base sequence is complementary to a portion of the base sequence of an APOC3 transcript, wherein the portion comprises 15 or more nucleobases.

75. The composition of claim 74, wherein oligonucleotides of the plurality share the same linkage phosphorus stereochemistry independently at five or more chiral internucleotidic linkages, and about 50%400% of all oligonucleotides in the composition that share the common base sequence are oligonucleotides of the plurality.

76. The composition of claim 74, wherein oligonucleotides of the plurality share the same linkage phosphorus stereochemistry independently at each phosphorothioate internucleotidic linkage.

77. The composition of claim 74, wherein oligonucleotides of the plurality are of the same constitution.

78. The composition of claim 76, wherein the composition is a liquid composition.

79. The composition of claim 74, wherein oligonucleotides of the plurality each independently comprise a targeting moiety (R^{CD}).

80. The composition of claim 79, wherein R^{CD} is

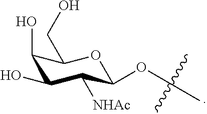

81. The composition of claim 79, wherein R^{CD} is

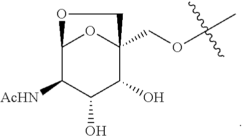 [SMILES: *OC[C@@]12CO[C@@H](O1)[C@H](NC(C)=O)[C@@H](O)[C@H]2O]

82. The composition of claim 79, wherein R^{CD} is of such a structure that R^{CD}—H is

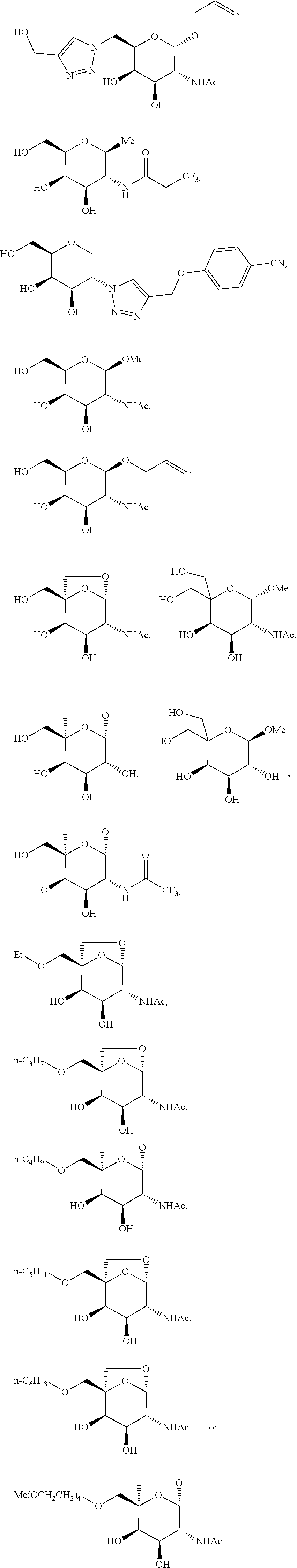

83. The composition of claim 79, wherein R^{CD} is connected to the oligonucleotide or oligonucleotides through a linker.

84. The composition of claim 83, wherein the linker has the structure of

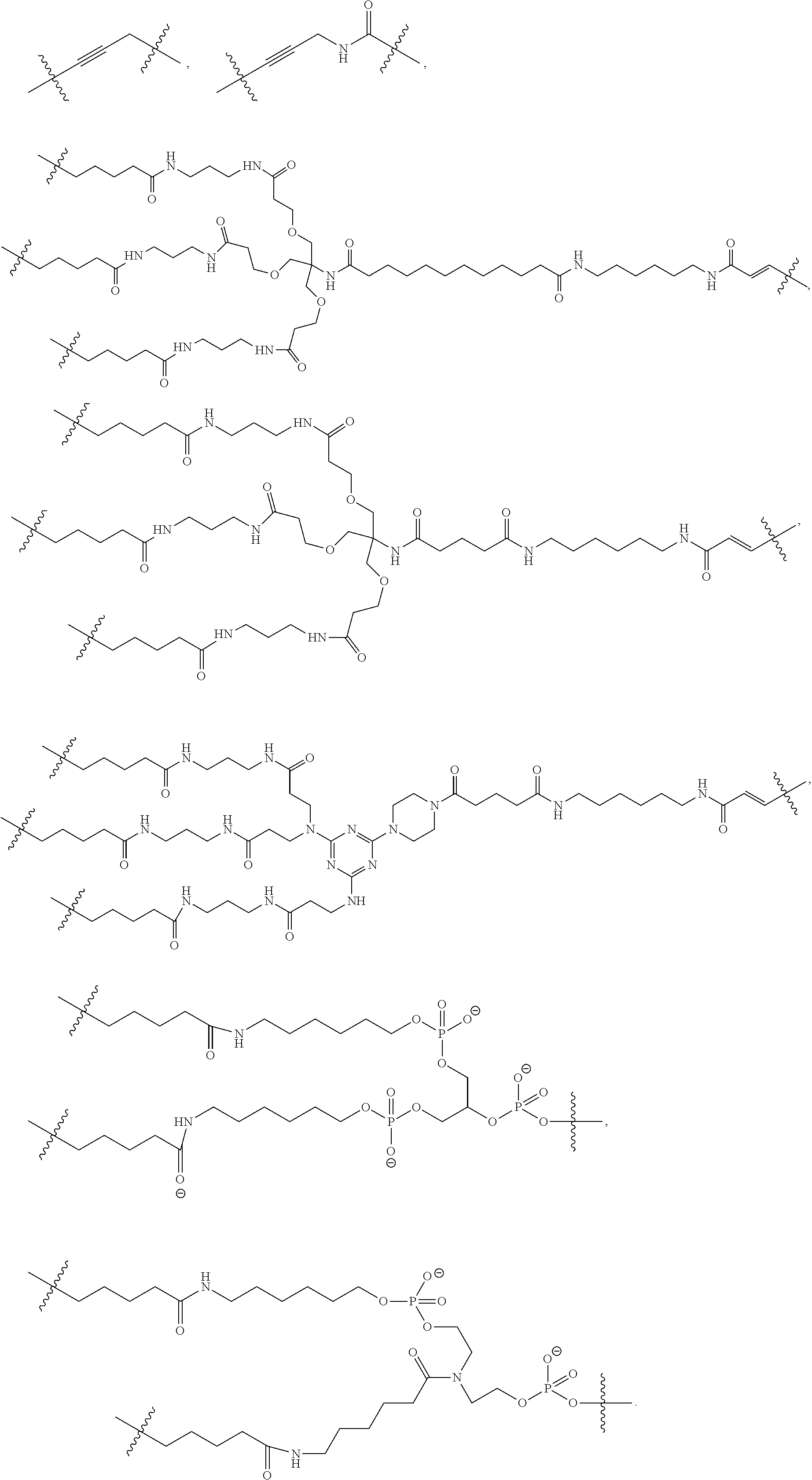

85. The composition of claim 79, wherein R^{CD} is selected from:

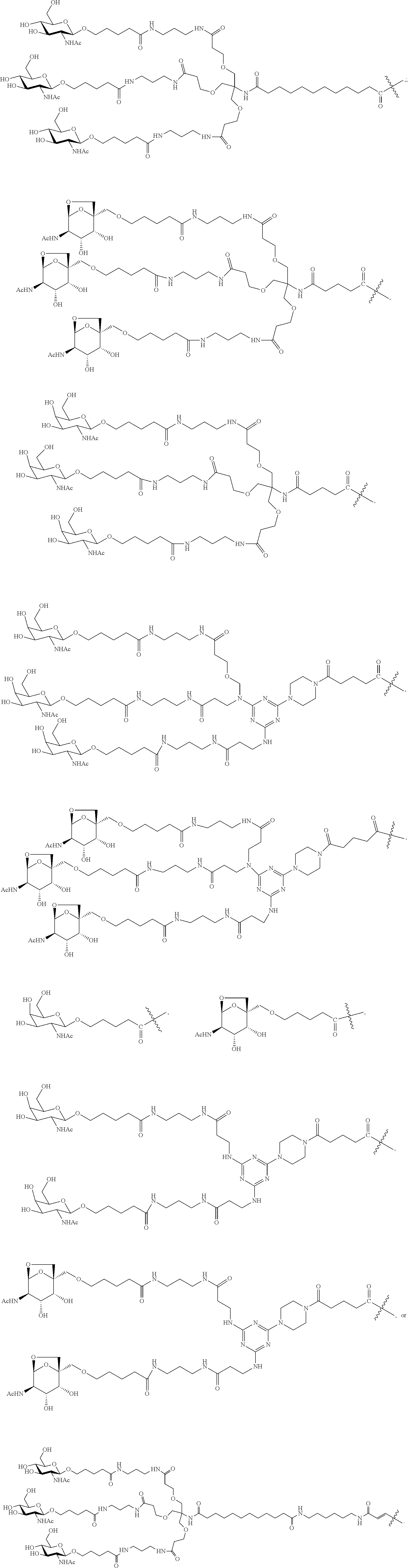

86. A method for treating fatty liver, nonalcoholic fatty liver disease, nonalcoholic steatohepatitis, nonalcoholic steatohepatitis with liver fibrosis, nonalcoholic steatohepatitis with cirrhosis, or nonalcoholic steatohepatitis with cirrhosis and hepatocellular carcinoma in humans comprising the step of administering to a human in need of such treatment a therapeutically effective amount of a composition of claim 74.
